(19) United States
(12) Patent Application Publication
(10) Pub. No.: US 2025/0248304 A1
(43) Pub. Date: Jul. 31, 2025

(54) COMPOUND, MATERIAL FOR ORGANIC ELECTROLUMINESCENT ELEMENTS, ORGANIC ELECTROLUMINESCENT ELEMENT, AND ELECTRONIC DEVICE

(71) Applicant: IDEMITSU KOSAN CO.,LTD., Tokyo (JP)

(72) Inventors: Tsukasa SAWATO, Chiyoda-ku, Tokyo (JP); Hiroaki ITOI, Chiyoda-ku, Tokyo (JP); Yusuke TAKAHASHI, Chiyoda-ku, Tokyo (JP); Shota TANAKA, Chiyoda-ku, Tokyo (JP); Takuto FUKAMI, Chiyoda-ku, Tokyo (JP)

(73) Assignee: IDEMITSU KOSAN CO.,LTD., Tokyo (JP)

(21) Appl. No.: 18/856,181

(22) PCT Filed: Apr. 12, 2023
(86) PCT No.: PCT/JP2023/014941

§ 371 Date: Oct. 11, 2024

(30) Foreign Application Priority Data
Apr. 15, 2022 (JP) 2022-067898

(51) Int. Cl. H10K 85/60; H10K 50/15

(52) U.S. Cl. CPC H10K 85/6574; H10K 50/15; H10K 85/631; H10K 85/6572; H10K 85/6576

(57) ABSTRACT

Provided are a compound and a material for an organic electroluminescent device, which further improve performance of an organic EL device; an organic electroluminescent device in which device performance is further improved; and an electronic device including such an organic electroluminescent device, which are a compound represented by the following formula (1) (each symbol in the formula is as defined in the specification); a material for an organic electroluminescent device including the compound; an organic electroluminescent device including the compound; and an electronic device including such an organic electroluminescent device.

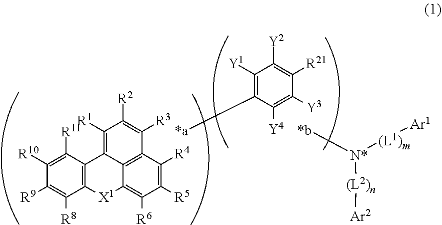

### TECHNICAL FIELD

The present invention relates to a compound, a material for an organic electroluminescent device, an organic electroluminescent device, and an electronic device including the organic electroluminescent device.

### BACKGROUND ART

In general, an organic electroluminescent device (hereinafter sometimes described as an “organic EL device”) is composed of an anode, a cathode, and an organic layer sandwiched between the anode and the cathode. When a voltage is applied between the anode and the cathode, electrons are injected from the cathode side and holes are injected from the anode side into a light emitting region, the injected electrons and holes are recombined in the light emitting region to generate an excited state, and light is emitted when the excited state returns to a ground state. Therefore, it is important to develop a material that efficiently transports electrons or holes to the light-emitting region and facilitates recombination of the electrons and holes in order to obtain a high-performance organic EL device.

PTLs 1 to 5 disclose compounds used as materials for organic electroluminescent devices.

### CITATION LIST

### Patent Literature

PTL 1: CN 111960954 A

PTL 2: CN 111848417 A

PTL 3: US 2017/092869 A

PTL 4: CN 113354611 A

PTL 5: US 2016/0149141 A

### SUMMARY OF INVENTION

### Technical Problem

Although many compounds for organic EL devices have been heretofore reported, there is still a need for compounds further improving performance of organic EL devices.

The present invention has been made to solve the above-described problem, and an object of the present invention is to provide: a compound and a material for organic electroluminescent devices, further improving performance of organic EL device; an organic EL device in which device performance is further improved; and an electronic device including such an organic EL device.

### Solution to Problem

As a result of intensive studies on the performance of an organic EL device including a novel compound, the present inventors have found that the performance of an organic EL device including a compound represented by the following formula (1) is further improved.

In one aspect, the present invention provides a compound represented by the following formula (1).

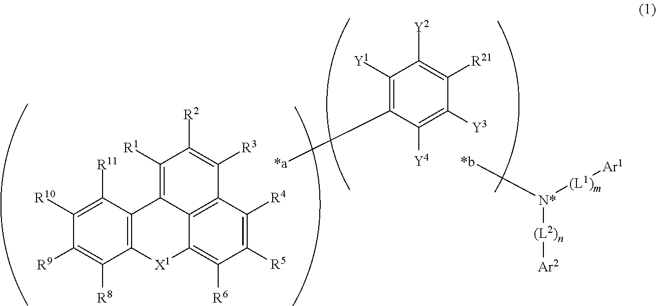

In formula (1),
X¹ is an oxygen atom or a sulfur atom.

One selected from R¹ to R⁶ and R⁸ to R¹¹ is a single bond bonded to *a.

R¹ to R⁶ and R⁸ to R¹¹ that are not the single bond are each independently a hydrogen atom, an unsubstituted alkyl group having 1 to 30 carbon atoms, or an unsubstituted aryl group having 6 to 12 ring carbon atoms.

Each combination of adjacent moieties among R¹ to R⁶ and R⁸ to R¹¹ that are not the single bond forms no ring, with the adjacent moieties not bonded to each other.

R²¹ is a hydrogen atom.

One selected from Y¹ to Y⁴ is a single bond bonded to *b.

Y¹ to Y⁴ that are not the single bond are hydrogen atoms.

N* is a central nitrogen atom.

m is 0 or 1.

n is 0 or 1.

When m is 0, Ar¹ is directly bonded to the central nitrogen atom *.

When n is 0, Ar² is directly bonded to the central nitrogen atom *.

L¹ and L² are each independently a substituted or unsubstituted phenylene group, a substituted or unsubstituted naphthylene group, or a substituted or unsubstituted biphenylene group.

A substituent of the phenylene group, the naphthylene group, and the biphenylene group which L¹ and L² may take is selected from an unsubstituted alkyl group having 1 to 6 carbon atoms and an aryl group having 6 to 12 ring carbon atoms, and substituents are not bonded to each other and form no ring.

Ar¹ and Ar² are each independently represented by any of the following formulae (1-a) to (1-d).

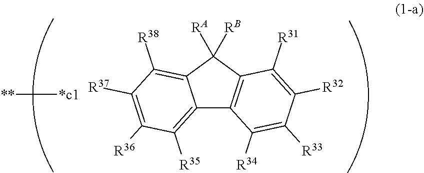

In formula (1-a),
** represents a bonding site to one or both of L¹ and L².

One selected from R³¹ to R³⁸, R^{A}, and R^{B} is a single bond bonded to *c1, or one selected from R^{A} and R^{B} is a divalent group bonded to *c1.

R³¹ to R³⁸ that are not the single bond are each independently a hydrogen atom, an unsubstituted alkyl group having 1 to 30 carbon atoms, or an unsubstituted aryl group having 6 to 12 ring carbon atoms.

One combination of adjacent moieties among R³¹ to R³⁸ that are not the single bond forms an unsubstituted benzene ring, with the adjacent moieties bonded to each other, or each combination of adjacent moieties among R³¹ to R³⁸ forms no ring, with the adjacent moieties not bonded to each other.

R^{A} and R^{B} that are not the single bond and are not a group bonded to *c1 are a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms, and at least one of R^{A} and R^{B} is a substituted or unsubstituted aryl group having 6 to 30 ring carbon atoms.

The divalent group bonded to *c1 represented by R^{A} and R^{B} is a substituted or unsubstituted alkylene group having 1 to 30 carbon atoms, a substituted or unsubstituted arylene group having 6 to 30 ring carbon atoms, or a substituted or unsubstituted heteroarylene group having 5 to 30 ring atoms.

R^{A} and R^{B} that are not the single bond and are not the group bonded to the *c1 are bonded to each other to form a ring, or are not bonded to each other and form no ring.

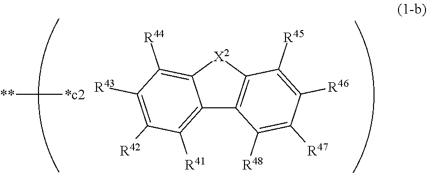

In formula (1-b),
** represents a bonding site to one or both of L¹ and L².

X² is an oxygen atom or a sulfur atom.

One selected from R⁴¹ to R⁴⁸ is a single bond bonded to *c2.

R⁴¹ to R⁴⁸ that are not the single bond are each independently a hydrogen atom, an unsubstituted alkyl group having 1 to 30 carbon atoms, or an unsubstituted aryl group having 6 to 12 ring carbon atoms.

One combination of adjacent moieties among R⁴¹ to R⁴⁸ that are not the single bond forms an unsubstituted benzene ring, with the adjacent moieties bonded to each other, or each combination of adjacent moieties among R⁴¹ to R⁴⁸ forms no ring, with the adjacent moieties not bonded to each other.

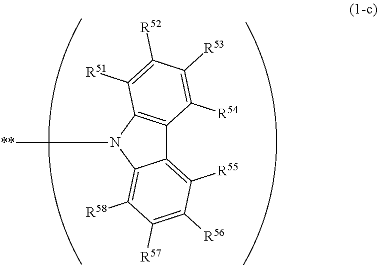

In formula (1-c),
** represents a bonding site to one or both of L¹ and L².

R⁵¹ to R⁵⁸ are each independently a hydrogen atom, an unsubstituted alkyl group having 1 to 30 carbon atoms, or an unsubstituted aryl group having 6 to 12 ring carbon atoms.

One combination of adjacent moieties among R⁵¹ to R⁵⁸ that are not the single bond forms an unsubstituted benzene ring, with the adjacent moieties bonded to each other, or each combination of adjacent moieties among R⁵¹ to R⁵⁸ forms no ring, with the adjacent moieties not bonded to each other.

**—Z (1-d)

In formula (1-d),
** represents a bonding site to one or both of L¹ and L².

Z is a hydrogen atom or a substituted or unsubstituted aryl group consisting only of a 6-membered ring and having 6 to 30 ring carbon atoms.

Substituents on the aryl group represented by Z are bonded to each other to form a monocyclic ring, are bonded to each other to form a condensed ring, or are not bonded to each other and form no ring.

The condensed ring is selected from a naphthalene ring, an anthracene ring, a phenanthrene ring, and a phenalene ring.

In another aspect, the present invention provides a material for an organic electroluminescent device including the compound represented by the formula (1).

In yet another aspect, the present invention provides an organic electroluminescent device including a cathode, an anode, and an organic layer between the cathode and the anode, in which the organic layer includes a light emitting layer, and at least one layer of the organic layer includes the compound represented by the formula (1).

In another aspect, the present invention provides an electronic device including the organic electroluminescent device.

### Advantageous Effects of Invention

An organic EL device including the compound represented by the formula (1) exhibits improved device performance.

### BRIEF DESCRIPTION OF DRAWINGS

FIG. 1 is a schematic diagram illustrating an example of a layer structure of an organic EL device according to one embodiment of the present invention.

FIG. 2 is a schematic view illustrating another example of the layer structure of the organic EL device according to one embodiment of the present invention.

FIG. 3 is a schematic view illustrating still another example of the layer structure of the organic EL device according to one embodiment of the present invention.

### DESCRIPTION OF EMBODIMENTS

### Definitions

In the description herein, the hydrogen atom encompasses isotopes thereof having different numbers of neutrons, i.e., a light hydrogen atom (protium), a heavy hydrogen atom (deuterium), and tritium.

In the description herein, the bonding site where the symbol, such as “R”, or “D” representing a deuterium atom is not shown is assumed to have a hydrogen atom, i.e., a protium atom, a deuterium atom, or a tritium atom, bonded thereto.

In the description herein, the number of ring carbon atoms shows the number of carbon atoms among the atoms constituting the ring itself of a compound having a structure including atoms bonded to form a ring (such as a monocyclic compound, a condensed ring compound, a bridged compound, a carbocyclic compound, and a heterocyclic compound). In the case where the ring is substituted by a substituent, the carbon atom contained in the substituent is not included in the number of ring carbon atoms. The same definition is applied to the “number of ring carbon atoms” described hereinafter unless otherwise indicated. For example, a benzene ring has 6 ring carbon atoms, a naphthalene ring has 10 ring carbon atoms, a pyridine ring has 5 ring carbon atoms, and a furan ring has 4 ring carbon atoms. For example, 9,9-diphenylfluorenyl group has 13 ring carbon atoms, and 9,9′-spirobifluorenyl group has 25 ring carbon atoms.

In the case where a benzene ring has, for example, an alkyl group substituted thereon as a substituent, the number of carbon atoms of the alkyl group is not included in the number of ring carbon atoms of the benzene ring. Accordingly, a benzene ring having an alkyl group substituted thereon has 6 ring carbon atoms. In the case where a naphthalene ring has, for example, an alkyl group substituted thereon as a substituent, the number of carbon atoms of the alkyl group is not included in the number of ring carbon atoms of the naphthalene ring. Accordingly, a naphthalene ring having an alkyl group substituted thereon has 10 ring carbon atoms.

In the description herein, the number of ring atoms shows the number of atoms constituting the ring itself of a compound having a structure including atoms bonded to form a ring (such as a monocyclic ring, a condensed ring, and a set of rings) (such as a monocyclic compound, a condensed ring compound, a bridged compound, a carbocyclic compound, and a heterocyclic compound). The atom that does not constitute the ring (such as a hydrogen atom terminating the bond of the atom constituting the ring) and, in the case where the ring is substituted by a substituent, the atom contained in the substituent are not included in the number of ring atoms. The same definition is applied to the “number of ring atoms” described hereinafter unless otherwise indicated. For example, a pyridine ring has 6 ring atoms, a quinazoline ring has 10 ring atoms, and a furan ring has 5 ring atoms. For example, the number of hydrogen atoms bonded to a pyridine ring or atoms constituting a substituent is not included in the number of ring atoms of the pyridine ring. Accordingly, a pyridine ring having a hydrogen atom or a substituent bonded thereto has 6 ring atoms. For example, the number of hydrogen atoms bonded to carbon atoms of a quinazoline ring or atoms constituting a substituent is not included in the number of ring atoms of the quinazoline ring. Accordingly, a quinazoline ring having a hydrogen atom or a substituent bonded thereto has 10 ring atoms.

In the description herein, the expression “having XX to YY carbon atoms” in the expression “substituted or unsubstituted ZZ group having XX to YY carbon atoms” means the number of carbon atoms of the unsubstituted ZZ group, and, in the case where the ZZ group is substituted, the number of carbon atoms of the substituent is not included. Herein, “YY” is larger than “XX”, “XX” represents an integer of 1 or more, and “YY” represents an integer of 2 or more.

In the description herein, the expression “having XX to YY atoms” in the expression “substituted or unsubstituted ZZ group having XX to YY atoms” means the number of atoms of the unsubstituted ZZ group, and, in the case where the ZZ group is substituted, the number of atoms of the substituent is not included. Herein, “YY” is larger than “XX”, “XX” represents an integer of 1 or more, and “YY” represents an integer of 2 or more.

In the description herein, an unsubstituted ZZ group means the case where the “substituted or unsubstituted ZZ group” is an “unsubstituted ZZ group”, and a substituted ZZ group means the case where the “substituted or unsubstituted ZZ group” is a “substituted ZZ group”.

In the description herein, the expression “unsubstituted” in the expression “substituted or unsubstituted ZZ group” means that hydrogen atoms in the ZZ group are not substituted by a substituent. The hydrogen atoms in the “unsubstituted ZZ group” each are a protium atom, a deuterium atom, or a tritium atom.

In the description herein, the expression “substituted” in the expression “substituted or unsubstituted ZZ group” means that one or more hydrogen atom in the ZZ group is substituted by a substituent. The expression “substituted” in the expression “BB group substituted by an AA group” similarly means that one or more hydrogen atom in the BB group is substituted by the AA group.

### Substituents in Description

The substituents described in the description herein will be explained. Each of the substituents described in the description herein will be defined as described hereinafter, unless otherwise indicated in the description.

In the description herein, the number of ring carbon atoms of the “unsubstituted aryl group” is 6 to 50, preferably 6 to 30, and more preferably 6 to 18, unless otherwise indicated in the description.

In the description herein, the number of ring atoms of the “unsubstituted heterocyclic group” is 5 to 50, preferably 5 to 30, and more preferably 5 to 18, unless otherwise indicated in the description.

In the description herein, the number of carbon atoms of the “unsubstituted alkyl group” is 1 to 50, preferably 1 to 20, and more preferably 1 to 6, unless otherwise indicated in the description.

In the description herein, the number of carbon atoms of the “unsubstituted alkenyl group” is 2 to 50, preferably 2 to 20, and more preferably 2 to 6, unless otherwise indicated in the description.

In the description herein, the number of carbon atoms of the “unsubstituted alkynyl group” is 2 to 50, preferably 2 to 20, and more preferably 2 to 6, unless otherwise indicated in the description.

In the description herein, the number of ring carbon atoms of the “unsubstituted cycloalkyl group” is 3 to 50, preferably 3 to 20, and more preferably 3 to 6, unless otherwise indicated in the description.

In the description herein, the number of ring carbon atoms of the “unsubstituted arylene group” is 6 to 50, preferably 6 to 30, and more preferably 6 to 18, unless otherwise indicated in the description.

In the description herein, the number of ring atoms of the “unsubstituted divalent heterocyclic group” is 5 to 50, preferably 5 to 30, and more preferably 5 to 18, unless otherwise indicated in the description.

In the description herein, the number of carbon atoms of the “unsubstituted alkylene group” is 1 to 50, preferably 1 to 20, and more preferably 1 to 6, unless otherwise indicated in the description.

### Substituted or Unsubstituted Aryl Group

In the description herein, specific examples (set of specific examples G1) of the “substituted or unsubstituted aryl group” include the unsubstituted aryl groups (set of specific examples G1A) and the substituted aryl groups (set of specific examples G1B) shown below. (Herein, the unsubstituted aryl group means the case where the “substituted or unsubstituted aryl group” is an “unsubstituted aryl group”, and the substituted aryl group means the case where the “substituted or unsubstituted aryl group” is a “substituted aryl group”.) In the description herein, the simple expression “aryl group” encompasses both the “unsubstituted aryl group” and the “substituted aryl group”.

The “substituted aryl group” means a group formed by substituting one or more hydrogen atom of the “unsubstituted aryl group” by a substituent. Examples of the “substituted aryl group” include groups formed by one or more hydrogen atom of each of the “unsubstituted aryl groups” in the set of specific examples G1A by a substituent, and the examples of the substituted aryl groups in the set of specific examples G1B. The examples of the “unsubstituted aryl group” and the examples of the “substituted aryl group” enumerated herein are mere examples, and the “substituted aryl group” in the description herein encompasses groups formed by substituting a hydrogen atom bonded to the carbon atom of the aryl group itself of each of the “substituted aryl groups” in the set of specific examples G1B by a substituent, and groups formed by substituting a hydrogen atom of the substituent of each of the “substituted aryl groups” in the set of specific examples G1B by a substituent.

### Unsubstituted Aryl Group (Set of Specific Examples G1A):

a phenyl group,
a p-biphenyl group,
a m-biphenyl group,
an o-biphenyl group,
a p-terphenyl-4-yl group,
a p-terphenyl-3-yl group,
a p-terphenyl-2-yl group,
a m-terphenyl-4-yl group,
a m-terphenyl-3-yl group,
a m-terphenyl-2-yl group,
a m-terphenyl-3′-yl group,
an o-terphenyl-4-yl group,
an o-terphenyl-3-yl group,
an o-terphenyl-2-yl group,
a 1-naphthyl group,
a 2-naphthyl group,
an anthryl group,
a benzanthryl group,
a phenanthryl group,
a benzophenanthryl group, a phenarenyl group,
a pyrenyl group,
a chrysenyl group,
a benzochrysenyl group,
a triphenylenyl group,
a benzotriphenylenyl group,
a tetracenyl group,
a pentacenyl group,
a fluorenyl group,
a 9,9′-spirobifluorenyl group,
a benzofluorenyl group,
a dibenzofluorenyl group,
a fluoranthenyl group,
a benzofluoranthenyl group,
a perylenyl group, and
monovalent aryl groups derived by removing one hydrogen atom from each of the ring structures represented by the following general formulae (TEMP-1) to (TEMP-15):

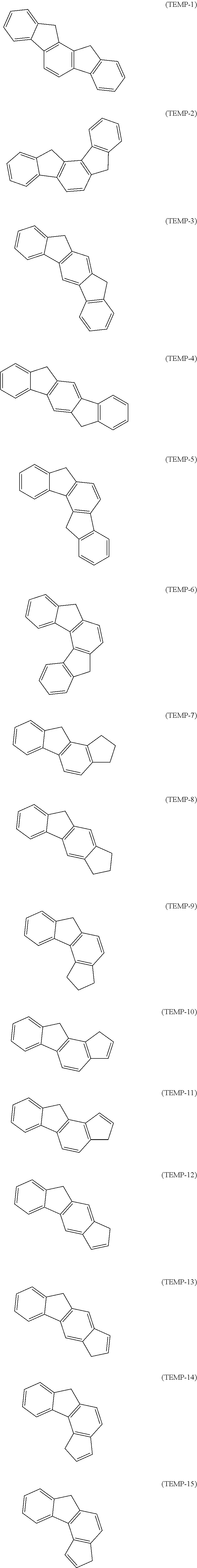

### Substituted Aryl Group (Set of Specific Examples G1B):

an o-tolyl group,
a m-tolyl group,
a p-tolyl group,
a p-xylyl group,
a m-xylyl group,
an o-xylyl group,
a p-isopropylphenyl group,
a m-isopropylphenyl group,
an o-isopropylphenyl group,
a p-t-butylphenyl group,
a m-t-butylphenyl group,
a o-t-butylphenyl group,
a 3,4,5-trimethylphenyl group,
a 9,9-dimethylfluorenyl group,
a 9,9-diphenylfluorenyl group,
a 9,9-bis(4-methylphenyl)fluorenyl group,
a 9,9-bis(4-isopropylphenyl)fluorenyl group,
a 9,9-bis(4-t-butylphenyl)fluorenyl group,
a cyanophenyl group,
a triphenylsilylphenyl group,
a trimethylsilylphenyl group,
a phenylnaphthyl group,
a naphthylphenyl group, and
groups formed by substituting one or more hydrogen atom of each of monovalent aryl groups derived from the ring structures represented by the general formulae (TEMP-1) to (TEMP-15) by a substituent.

### Substituted or Unsubstituted Heterocyclic Group

In the description herein, the “heterocyclic group” means a cyclic group containing at least one hetero atom in the ring atoms. Specific examples of the hetero atom include a nitrogen atom, an oxygen atom, a sulfur atom, a silicon atom, a phosphorus atom, and a boron atom. In the description herein, the “heterocyclic group” is a monocyclic group or a condensed ring group.

In the description herein, the “heterocyclic group” is an aromatic heterocyclic group or a non-aromatic heterocyclic group.

In the description herein, specific examples (set of specific examples G2) of the “substituted or unsubstituted heterocyclic group” include the unsubstituted heterocyclic groups (set of specific examples G2A) and the substituted heterocyclic groups (set of specific examples G2B) shown below. (Herein, the unsubstituted heterocyclic group means the case where the “substituted or unsubstituted heterocyclic group” is an “unsubstituted heterocyclic group”, and the substituted heterocyclic group means the case where the “substituted or unsubstituted heterocyclic group” is a “substituted heterocyclic group”.) In the description herein, the simple expression “heterocyclic group” encompasses both the “unsubstituted heterocyclic group” and the “substituted heterocyclic group”.

The “substituted heterocyclic group” means a group formed by substituting one or more hydrogen atom of the “unsubstituted heterocyclic group” by a substituent. Specific examples of the “substituted heterocyclic group” include groups formed by substituting a hydrogen atom of each of the “unsubstituted heterocyclic groups” in the set of specific examples G2A by a substituent, and the examples of the substituted heterocyclic groups in the set of specific examples G2B. The examples of the “unsubstituted heterocyclic group” and the examples of the “substituted heterocyclic group” enumerated herein are mere examples, and the “substituted heterocyclic group” in the description herein encompasses groups formed by substituting a hydrogen atom bonded to the ring atom of the heterocyclic group itself of each of the “substituted heterocyclic groups” in the set of specific examples G2B by a substituent, and groups formed by substituting a hydrogen atom of the substituent of each of the “substituted heterocyclic groups” in the set of specific examples G2B by a substituent.

The set of specific examples G2A includes, for example, the unsubstituted heterocyclic group containing a nitrogen atom (set of specific examples G2A1), the unsubstituted heterocyclic group containing an oxygen atom (set of specific examples G2A2), the unsubstituted heterocyclic group containing a sulfur atom (set of specific examples G2A3), and monovalent heterocyclic groups derived by removing one hydrogen atom from each of the ring structures represented by the following general formulae (TEMP-16) to (TEMP-33) (set of specific examples G2A4).

The set of specific examples G2B includes, for example, the substituted heterocyclic groups containing a nitrogen atom (set of specific examples G2B1), the substituted heterocyclic groups containing an oxygen atom (set of specific examples G2B2), the substituted heterocyclic groups containing a sulfur atom (set of specific examples G2B3), and groups formed by substituting one or more hydrogen atom of each of monovalent heterocyclic groups derived from the ring structures represented by the following general formulae (TEMP-16) to (TEMP-33) by a substituent (set of specific examples G2B4).

Unsubstituted Heterocyclic Group containing Nitrogen Atom (Set of Specific Examples G2A1):
a pyrrolyl group,
an imidazolyl group,
a pyrazolyl group,
a triazolyl group,
a tetrazolyl group,
an oxazolyl group,
an isoxazolyl group,
an oxadiazolyl group,
a thiazolyl group,
an isothiazolyl group,
a thiadiazolyl group,
a pyridyl group,
a pyridazinyl group,
a pyrimidinyl group,
a pyrazinyl group,
a triazinyl group,
an indolyl group,
an isoindolyl group,
an indolizinyl group, a quinolizinyl group,
a quinolyl group,
an isoquinolyl group,
a cinnolinyl group,
a phthalazinyl group,
a quinazolinyl group,
a quinoxalinyl group,
a benzimidazolyl group,
an indazolyl group,
a phenanthrolinyl group,
a phenanthridinyl group,
an acridinyl group,
a phenazinyl group,
a carbazolyl group,
a benzocarbazolyl group,
a morpholino group,
a phenoxazinyl group,
a phenothiazinyl group,
an azacarbazolyl group, and a diazacarbazolyl group.
   Unsubstituted Heterocyclic Group containing Oxygen Atom (Set of Specific Examples G2A2):
a furyl group,
an oxazolyl group,
an isoxazolyl group,
an oxadiazolyl group,
a xanthenyl group,
a benzofuranyl group,
an isobenzofuranyl group,
a dibenzofuranyl group,
a naphthobenzofuranyl group,
a benzoxazolyl group,
a benzisoxazolyl group,
a phenoxazinyl group,
a morpholino group,
a dinaphthofuranyl group,
an azadibenzofuranyl group,
a diazadibenzofuranyl group,
an azanaphthobenzofuranyl group, and
a diazanaphthobenzofuranyl group.
   Unsubstituted Heterocyclic Group containing Sulfur Atom (Set of Specific Examples G2A3):
a thienyl group,
a thiazolyl group,
an isothiazolyl group,
a thiadiazolyl group,
a benzothiophenyl group (benzothienyl group),
an isobenzothiophenyl group (isobenzothienyl group),
a dibenzothiophenyl group (dibenzothienyl group),
a naphthobenzothiophenyl group (naphthobenzothienyl group),
a benzothiazolyl group, a benzisothiazolyl group,
a phenothiazinyl group,
a dinaphthothiophenyl group (dinaphthothienyl group),
an azadibenzothiophenyl group (azadibenzothienyl group),
a diazadibenzothiophenyl group (diazadibenzothienyl group),
an azanaphthobenzothiophenyl group (azanaphthobenzothienyl group), and
a diazanaphthobenzothiophenyl group (diazanaphthobenzothienyl group).

Monovalent Heterocyclic Group derived by removing One Hydrogen Atom from Ring Structures represented by General Formulae (TEMP-16) to (TEMP-33) (Set of Specific Examples G2A4)

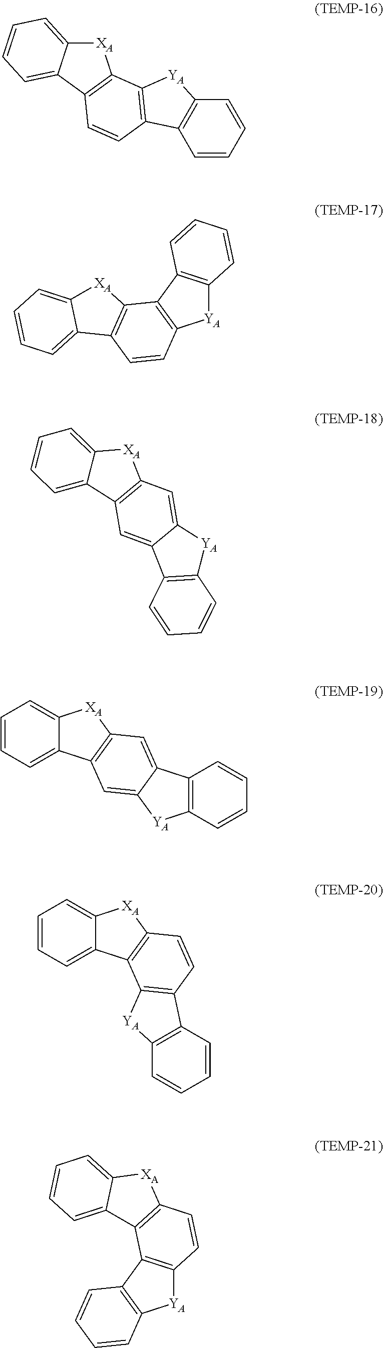

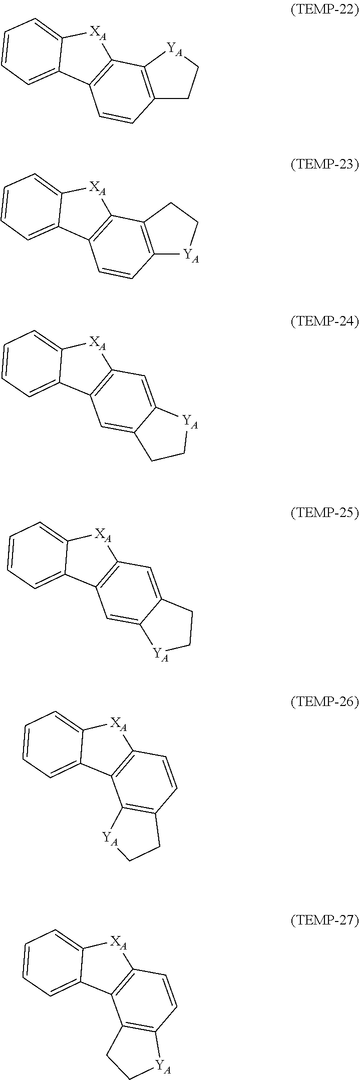

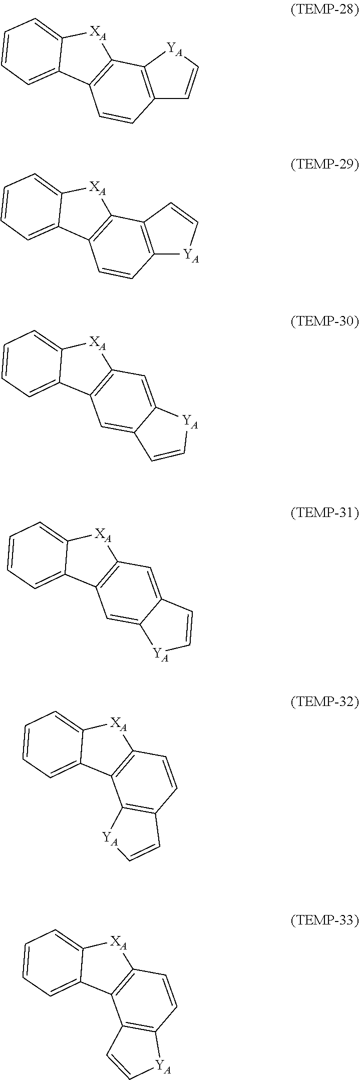

In the general formulae (TEMP-16) to (TEMP-33), X_{A} and Y_{A} each independently represent an oxygen atom, a sulfur atom, NH, or CH₂, provided that at least one of X_{A} and Y_{A} represents an oxygen atom, a sulfur atom, or NH.

In the general formulae (TEMP-16) to (TEMP-33), in the case where at least one of X_{A} and Y_{A} represents NH or CH₂, the monovalent heterocyclic groups derived from the ring structures represented by the general formulae (TEMP-16) to (TEMP-33) include monovalent groups formed by removing one hydrogen atom from the NH or CH₂.

Substituted Heterocyclic Group containing Nitrogen Atom (Set of Specific Examples G2B1):
a (9-phenyl)carbazolyl group,
a (9-biphenylyl)carbazolyl group,
a (9-phenyl)phenylcarbazolyl group,
a (9-naphthyl)carbazolyl group,
a diphenylcarbazol-9-yl group,
a phenylcarbazol-9-yl group,
a methylbenzimidazolyl group,
an ethylbenzimidazolyl group,
a phenyltriazinyl group,
a biphenyltriazinyl group,
a diphenyltriazinyl group,
a phenylquinazolinyl group, and a biphenylquinazolinyl group.
   Substituted Heterocyclic Group containing Oxygen Atom (Set of Specific Examples G2B2):
a phenyldibenzofuranyl group,
a methyldibenzofuranyl group,
   a t-butyldibenzofuranyl group, and
   a monovalent residual group of spiro[9H-xanthene-9,9′-[9H]fluorene].
      Substituted Heterocyclic Group containing Sulfur Atom (Set of Specific Examples G2B3):
a phenyldibenzothiophenyl group,
a methyldibenzothiophenyl group,
a t-butyldibenzothiophenyl group, and
a monovalent residual group of spiro[9H-thioxanthene-9,9′-[9H]fluorene].

Group formed by substituting one or more Hydrogen Atom of Monovalent Heterocyclic Group derived from Ring Structures represented by General Formulae (TEMP-16) to (TEMP-33) by Substituent (Set of Specific Examples G2B4)

The “one or more hydrogen atom of the monovalent heterocyclic group” means one or more hydrogen atom selected from the hydrogen atom bonded to the ring carbon atom of the monovalent heterocyclic group, the hydrogen atom bonded to the nitrogen atom in the case where at least one of X_{A} and Y_{A} represents NH, and the hydrogen atom of the methylene group in the case where one of X_{A} and Y_{A} represents CH₂.

### Substituted or Unsubstituted Alkyl Group

In the description herein, specific examples (set of specific examples G3) of the “substituted or unsubstituted alkyl group” include the unsubstituted alkyl groups (set of specific examples G3A) and the substituted alkyl groups (set of specific examples G3B) shown below. (Herein, the unsubstituted alkyl group means the case where the “substituted or unsubstituted alkyl group” is an “unsubstituted alkyl group”, and the substituted alkyl group means the case where the “substituted or unsubstituted alkyl group” is a “substituted alkyl group”.) In the description herein, the simple expression “alkyl group” encompasses both the “unsubstituted alkyl group” and the “substituted alkyl group”.

The “substituted alkyl group” means a group formed by substituting one or more hydrogen atom of the “unsubstituted alkyl group” by a substituent. Specific examples of the “substituted alkyl group” include groups formed by substituting one or more hydrogen atom of each of the “unsubstituted alkyl groups” (set of specific examples G3A) by a substituent, and the examples of the substituted alkyl groups (set of specific examples G3B). In the description herein, the alkyl group in the “unsubstituted alkyl group” means a chain-like alkyl group. Accordingly, the “unsubstituted alkyl group” encompasses an “unsubstituted linear alkyl group” and an “unsubstituted branched alkyl group”. The examples of the “unsubstituted alkyl group” and the examples of the “substituted alkyl group” enumerated herein are mere examples, and the “substituted alkyl group” in the description herein encompasses groups formed by substituting a hydrogen atom of the alkyl group itself of each of the “substituted alkyl groups” in the set of specific examples G3B by a substituent, and groups formed by substituting a hydrogen atom of the substituent of each of the “substituted alkyl groups” in the set of specific examples G3B by a substituent.

### Unsubstituted Alkyl Group (Set of Specific Examples G3A):

a methyl group,
an ethyl group,
a n-propyl group,
an isopropyl group,
a n-butyl group,
an isobutyl group,
a s-butyl group, and
a t-butyl group.

### Substituted Alkyl Group (Set of Specific Examples G3B):

a heptafluoropropyl group (including isomers),
a pentafluoroethyl group,
a 2,2,2-trifluoroethyl group, and
a trifluoromethyl group.

### Substituted or Unsubstituted Alkenyl Group

In the description herein, specific examples (set of specific examples G4) of the “substituted or unsubstituted alkenyl group” include the unsubstituted alkenyl groups (set of specific examples G4A) and the substituted alkenyl groups (set of specific examples G4B) shown below. (Herein, the unsubstituted alkenyl group means the case where the “substituted or unsubstituted alkenyl group” is an “unsubstituted alkenyl group”, and the substituted alkenyl group means the case where the “substituted or unsubstituted alkenyl group” is a “substituted alkenyl group”.) In the description herein, the simple expression “alkenyl group” encompasses both the “unsubstituted alkenyl group” and the “substituted alkenyl group”.

The “substituted alkenyl group” means a group formed by substituting one or more hydrogen atom of the “unsubstituted alkenyl group” by a substituent. Specific examples of the “substituted alkenyl group” include the “unsubstituted alkenyl groups” (set of specific examples G4A) that each have a substituent, and the examples of the substituted alkenyl groups (set of specific examples G4B). The examples of the “unsubstituted alkenyl group” and the examples of the “substituted alkenyl group” enumerated herein are mere examples, and the “substituted alkenyl group” in the description herein encompasses groups formed by substituting a hydrogen atom of the alkenyl group itself of each of the “substituted alkenyl groups” in the set of specific examples G4B by a substituent, and groups formed by substituting a hydrogen atom of the substituent of each of the “substituted alkenyl groups” in the set of specific examples G4B by a substituent.

### Unsubstituted Alkenyl Group (Set of Specific Examples G4A):

a vinyl group,
an allyl group,
a 1-butenyl group,
a 2-butenyl group, and
a 3-butenyl group.

### Substituted Alkenyl Group (Set of Specific Examples G4B):

a 1,3-butanedienyl group,
a 1-methylvinyl group,
a 1-methylallyl group,
a 1,1-dimethylallyl group,
a 2-methylallyl group, and
a 1,2-dimethylallyl group.

### Substituted or Unsubstituted Alkynyl Group

In the description herein, specific examples (set of specific examples G5) of the “substituted or unsubstituted alkynyl group” include the unsubstituted alkynyl group (set of specific examples G5A) shown below. (Herein, the unsubstituted alkynyl group means the case where the “substituted or unsubstituted alkynyl group” is an “unsubstituted alkynyl group”.) In the description herein, the simple expression “alkynyl group” encompasses both the “unsubstituted alkynyl group” and the “substituted alkynyl group”.

The “substituted alkynyl group” means a group formed by substituting one or more hydrogen atom of the “unsubstituted alkynyl group” by a substituent. Specific examples of the “substituted alkynyl group” include groups formed by substituting one or more hydrogen atom of the “unsubstituted alkynyl group” (set of specific examples G5A) by a substituent.

### Unsubstituted Alkynyl Group (Set of Specific Examples G5A):

an ethynyl group.

### Substituted or Unsubstituted Cycloalkyl Group

In the description herein, specific examples (set of specific examples G6) of the “substituted or unsubstituted cycloalkyl group” include the unsubstituted cycloalkyl groups (set of specific examples G6A) and the substituted cycloalkyl group (set of specific examples G6B) shown below. (Herein, the unsubstituted cycloalkyl group means the case where the “substituted or unsubstituted cycloalkyl group” is an “unsubstituted cycloalkyl group”, and the substituted cycloalkyl group means the case where the “substituted or unsubstituted cycloalkyl group” is a “substituted cycloalkyl group”.) In the description herein, the simple expression “cycloalkyl group” encompasses both the “unsubstituted cycloalkyl group” and the “substituted cycloalkyl group”.

The “substituted cycloalkyl group” means a group formed by substituting one or more hydrogen atom of the “unsubstituted cycloalkyl group” by a substituent. Specific examples of the “substituted cycloalkyl group” include groups formed by substituting one or more hydrogen atom of each of the “unsubstituted cycloalkyl groups” (set of specific examples G6A) by a substituent, and the example of the substituted cycloalkyl group (set of specific examples G6B1). The examples of the “unsubstituted cycloalkyl group” and the examples of the “substituted cycloalkyl group” enumerated herein are mere examples, and the “substituted cycloalkyl group” in the description herein encompasses groups formed by substituting one or more hydrogen atom bonded to the carbon atoms of the cycloalkyl group itself of the “substituted cycloalkyl group” in the set of specific examples G6B by a substituent, and groups formed by substituting a hydrogen atom of the substituent of the “substituted cycloalkyl group” in the set of specific examples G6B by a substituent.

### Unsubstituted Cycloalkyl Group (Set of Specific Examples G6A):

a cyclopropyl group,
a cyclobutyl group,
a cyclopentyl group,
a cyclohexyl group,
a 1-adamantyl group,
a 2-adamantyl group,
a 1-norbornyl group, and
a 2-norbornyl group.

### Substituted Cycloalkyl Group (Set of Specific Examples G6B):

a 4-methylcyclohexyl group.
   Group represented by —Si(R₉₀₁)(R₉₀₂)(R₉₀₃)

In the description herein, specific examples (set of specific examples G7) of the group represented by —Si(R₉₀₁)(R₉₀₂)(R₉₀₃) include:
—Si(G1)(G1)(G1),
—Si(G1)(G2)(G2),
—Si(G1)(G1)(G2),
—Si(G2)(G2)(G2),
—Si(G3)(G3)(G3), and
—Si(G6)(G6)(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.

Plural groups represented by G1 in —Si(G1)(G1)(G1) are the same as or different from each other.

Plural groups represented by G2 in —Si(G1)(G2)(G2) are the same as or different from each other.

Plural groups represented by G1 in —Si(G1)(G1)(G2) are the same as or different from each other.

Plural groups represented by G2 in —Si(G2)(G2)(G2) are the same as or different from each other.

Plural groups represented by G3 in —Si(G3)(G3)(G3) are the same as or different from each other.

Plural groups represented by G6 in —Si(G6)(G6)(G6) are the same as or different from each other.

### Group Represented by —O—(R₉₀₄)

In the description herein, specific examples (set of specific examples G8) of the group represented by —O—(R₉₀₄) include:
—O(G1),
—O(G2),
—O(G3), and
—O(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.

### Group Represented by —S—(R₉₀₅)

In the description herein, specific examples (set of specific examples G9) of the group represented by —S—(R₉₀₅) include:
—S(G1),
—S(G2),
—S(G3), and
—S(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.
   Group Represented by —N(R₉₀₆)(R₉₀₇)

In the description herein, specific examples (set of specific examples G10) of the group represented by —N(R₉₀₆)(R₉₀₇) include:
—N(G1)(G1),
—N(G2)(G2),
—N(G1)(G2),
—N(G3)(G3), and
—N(G6)(G6). Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.

Plural groups represented by G1 in —N(G1)(G1) are the same as or different from each other.

Plural groups represented by G2 in —N(G2)(G2) are the same as or different from each other.

Plural groups represented by G3 in —N(G3)(G3) are the same as or different from each other.

Plural groups represented by G6 in —N(G6)(G6) are the same as or different from each other.

### Halogen Atom

In the description herein, specific examples (set of specific examples G11) of the “halogen atom” include a fluorine atom, a chlorine atom, a bromine atom, and an iodine atom.

### Substituted or Unsubstituted Fluoroalkyl Group

In the description herein, the “substituted or unsubstituted fluoroalkyl group” means a group formed by substituting at least one hydrogen atom bonded to the carbon atom constituting the alkyl group in the “substituted or unsubstituted alkyl group” by a fluorine atom, and encompasses a group formed by substituting all the hydrogen atoms bonded to the carbon atoms constituting the alkyl group in the “substituted or unsubstituted alkyl group” by fluorine atoms (i.e., a perfluoroalkyl group). The number of carbon atoms of the “unsubstituted fluoroalkyl group” is 1 to 50, preferably 1 to 30, and more preferably 1 to 18, unless otherwise indicated in the description. The “substituted fluoroalkyl group” means a group formed by substituting one or more hydrogen atom of the “fluoroalkyl group” by a substituent. In the description herein, the “substituted fluoroalkyl group” encompasses a group formed by substituting one or more hydrogen atom bonded to the carbon atom of the alkyl chain in the “substituted fluoroalkyl group” by a substituent, and a group formed by substituting one or more hydrogen atom of the substituent in the “substituted fluoroalkyl group” by a substituent. Specific examples of the “unsubstituted fluoroalkyl group” include examples of groups formed by substituting one or more hydrogen atom in each of the “alkyl group” (set of specific examples G3) by a fluorine atom.

### Substituted or Unsubstituted Haloalkyl Group

In the description herein, the “substituted or unsubstituted haloalkyl group” means a group formed by substituting at least one hydrogen atom bonded to the carbon atom constituting the alkyl group in the “substituted or unsubstituted alkyl group” by a halogen atom, and encompasses a group formed by substituting all the hydrogen atoms bonded to the carbon atoms constituting the alkyl group in the “substituted or unsubstituted alkyl group” by halogen atoms. The number of carbon atoms of the “unsubstituted haloalkyl group” is 1 to 50, preferably 1 to 30, and more preferably 1 to 18, unless otherwise indicated in the description. The “substituted haloalkyl group” means a group formed by substituting one or more hydrogen atom of the “haloalkyl group” by a substituent. In the description herein, the “substituted haloalkyl group” encompasses a group formed by substituting one or more hydrogen atom bonded to the carbon atom of the alkyl chain in the “substituted haloalkyl group” by a substituent, and a group formed by substituting one or more hydrogen atom of the substituent in the “substituted haloalkyl group” by a substituent. Specific examples of the “unsubstituted haloalkyl group” include examples of groups formed by substituting one or more hydrogen atom in each of the “alkyl group” (set of specific examples G3) by a halogen atom. A haloalkyl group may be referred to as a halogenated alkyl group in some cases.

### Substituted or Unsubstituted Alkoxy Group

In the description herein, specific examples of the “substituted or unsubstituted alkoxy group” include a group represented by —O(G3), wherein G3 represents the “substituted or unsubstituted alkyl group” described in the set of specific examples G3. The number of carbon atoms of the “unsubstituted alkoxy group” is 1 to 50, preferably 1 to 30, and more preferably 1 to 18, unless otherwise indicated in the description.

### Substituted or Unsubstituted Alkylthio Group

In the description herein, specific examples of the “substituted or unsubstituted alkylthio group” include a group represented by —S(G3), wherein G3 represents the “substituted or unsubstituted alkyl group” described in the set of specific examples G3. The number of carbon atoms of the “unsubstituted alkylthio group” is 1 to 50, preferably 1 to 30, and more preferably 1 to 18, unless otherwise indicated in the description.

### Substituted or Unsubstituted Aryloxy Group

In the description herein, specific examples of the “substituted or unsubstituted aryloxy group” include a group represented by —O(G1), wherein G1 represents the “substituted or unsubstituted aryl group” described in the set of specific examples G1. The number of ring carbon atoms of the “unsubstituted aryloxy group” is 6 to 50, preferably 6 to 30, and more preferably 6 to 18, unless otherwise indicated in the description.

### Substituted or Unsubstituted Arylthio Group

In the description herein, specific examples of the “substituted or unsubstituted arylthio group” include a group represented by —S(G1), wherein G1 represents the “substituted or unsubstituted aryl group” described in the set of specific examples G1. The number of ring carbon atoms of the “unsubstituted arylthio group” is 6 to 50, preferably 6 to 30, and more preferably 6 to 18, unless otherwise indicated in the description.

### Substituted or Unsubstituted Trialkylsilyl Group

In the description herein, specific examples of the “trialkylsilyl group” include a group represented by —Si(G3)(G3)(G3), wherein G3 represents the “substituted or unsubstituted alkyl group” described in the set of specific examples G3. Plural groups represented by G3 in —Si(G3)(G3)(G3) are the same as or different from each other. The number of carbon atoms of each of alkyl groups of the “substituted or unsubstituted trialkylsilyl group” is 1 to 50, preferably 1 to 20, and more preferably 1 to 6, unless otherwise indicated in the description.

### Substituted or Unsubstituted Aralkyl Group

In the description herein, specific examples of the “substituted or unsubstituted aralkyl group” include a group represented by -(G3)-(G1), wherein G3 represents the “substituted or unsubstituted alkyl group” described in the set of specific examples G3, and G1 represents the “substituted or unsubstituted aryl group” described in the set of specific examples G1. Accordingly, the “aralkyl group” is a group formed by substituting a hydrogen atom of an “alkyl group” by an “aryl group” as a substituent, and is one embodiment of the “substituted alkyl group”. The “unsubstituted aralkyl group” is an “unsubstituted alkyl group” that is substituted by an “unsubstituted aryl group”, and the number of carbon atoms of the “unsubstituted aralkyl group” is 7 to 50, preferably 7 to 30, and more preferably 7 to 18, unless otherwise indicated in the description.

Specific examples of the “substituted or unsubstituted aralkyl group” include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-β-naphthylethyl group, a 2-β-naphthylethyl group, a 1-β-naphthylisopropyl group, and a 2-β-naphthylisopropyl group.

In the description herein, the substituted or unsubstituted aryl group is preferably a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, a 9,9-dimethylfluorenyl group, a 9,9-diphenylfluorenyl group, and the like, unless otherwise indicated in the description.

In the description herein, the substituted or unsubstituted heterocyclic group is preferably a pyridyl group, a pyrimidinyl group, a triazinyl group, a quinolyl group, an isoquinolyl group, a quinazolinyl group, a benzimidazolyl group, a phenanthrolinyl group, a carbazolyl group (e.g., a 1-carbazolyl group, a 2-carbazolyl group, a 3-carbazolyl group, a 4-carbazolyl group, or a 9-carbazolyl group), a benzocarbazolyl group, an azacarbazolyl group, a diazacarbazolyl group, a dibenzofuranyl group, a naphthobenzofuranly group, an azadibenzofuranyl group, a diazadibenzofuranyl group, a dibenzothiophenyl group, a naphthobenzothiophenyl group, an azadibenzothiophenyl group, a diazadibenzothiophenyl group, a (9-phenyl)carbazolyl group (e.g., a (9-phenyl)carbazol-1-yl group, a (9-phenyl)carbazol-2-yl group, a (9-phenyl)carbazol-3-yl group, or a (9-phenyl)carbazol-4-yl group), a (9-biphenylyl)carbazolyl group, a (9-phenyl)phenylcarbazolyl group, a diphenylcarbazol-9-yl group, a phenylcarbazol-9-yl group, a phenyltriazinyl group, a biphenylyltriazinyl group, a diphenyltriazinyl group, a phenyldibenzofuranyl group, a phenyldibenzothiophenyl group, and the like, unless otherwise indicated in the description.

In the description herein, the carbazolyl group is specifically any one of the following groups unless otherwise indicated in the description.

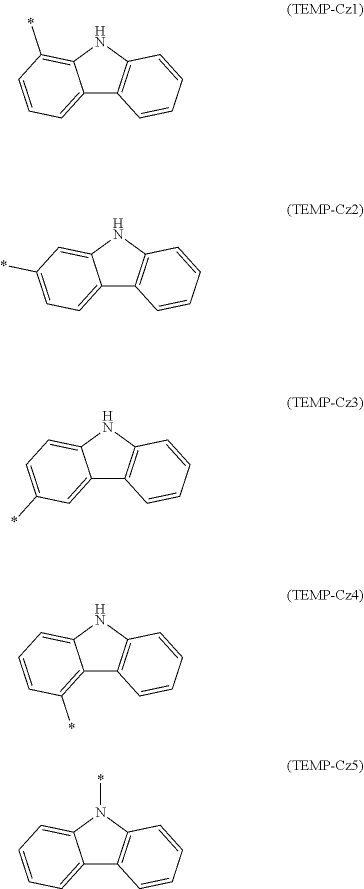 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

In the description herein, the (9-phenyl)carbazolyl group is specifically any one of the following groups unless otherwise indicated in the description.

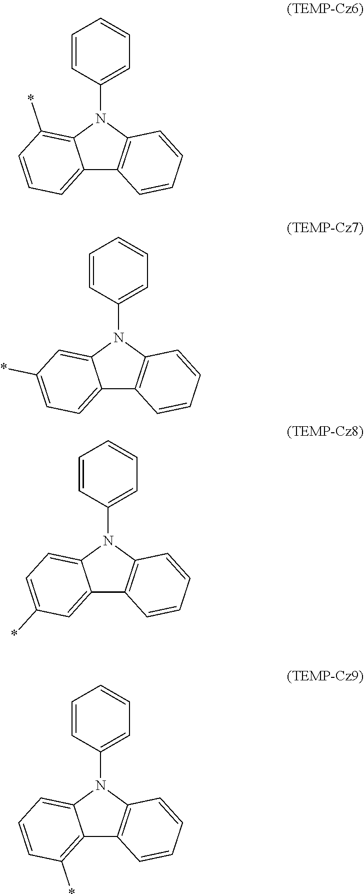

In the general formulae (TEMP-Cz1) to (TEMP-Cz9), * represents a bonding site.

In the description herein, the dibenzofuranyl group and the dibenzothiophenyl group are specifically any one of the following groups unless otherwise indicated in the description.

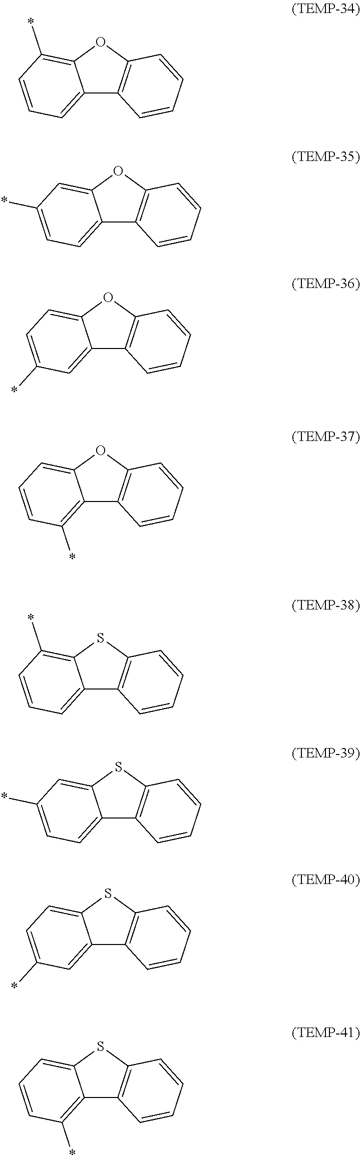

In the general formulae (TEMP-34) to (TEMP-41), * represents a bonding site.

In the description herein, the substituted or unsubstituted alkyl group is preferably a methyl group, an ethyl group, a propyl group, an isopropyl group, a n-butyl group, an isobutyl group, a t-butyl group, or the like unless otherwise indicated in the description.

### Substituted or Unsubstituted Arylene Group

In the description herein, the “substituted or unsubstituted arylene group” is a divalent group derived by removing one hydrogen atom on the aryl ring from the “substituted or unsubstituted aryl group” described above unless otherwise indicated in the description. Specific examples (set of specific examples G12) of the “substituted or unsubstituted arylene group” include divalent groups derived by removing one hydrogen atom on the aryl ring from the “substituted or unsubstituted aryl groups” described in the set of specific examples G1.

### Substituted or Unsubstituted Divalent Heterocyclic Group

In the description herein, the “substituted or unsubstituted divalent heterocyclic group” is a divalent group derived by removing one hydrogen atom on the heterocyclic ring from the “substituted or unsubstituted heterocyclic group” described above unless otherwise indicated in the description. Specific examples (set of specific examples G13) of the “substituted or unsubstituted divalent heterocyclic group” include divalent groups derived by removing one hydrogen atom on the heterocyclic ring from the “substituted or unsubstituted heterocyclic groups” described in the set of specific examples G2.

### Substituted or Unsubstituted Alkylene Group

In the description herein, the “substituted or unsubstituted alkylene group” is a divalent group derived by removing one hydrogen atom on the alkyl chain from the “substituted or unsubstituted alkyl group” described above unless otherwise indicated in the description. Specific examples (set of specific examples G14) of the “substituted or unsubstituted alkylene group” include divalent groups derived by removing one hydrogen atom on the alkyl chain from the “substituted or unsubstituted alkyl groups” described in the set of specific examples G3.

In the description herein, the substituted or unsubstituted arylene group is preferably any one of the groups represented by the following general formulae (TEMP-42) to (TEMP-68) unless otherwise indicated in the description.

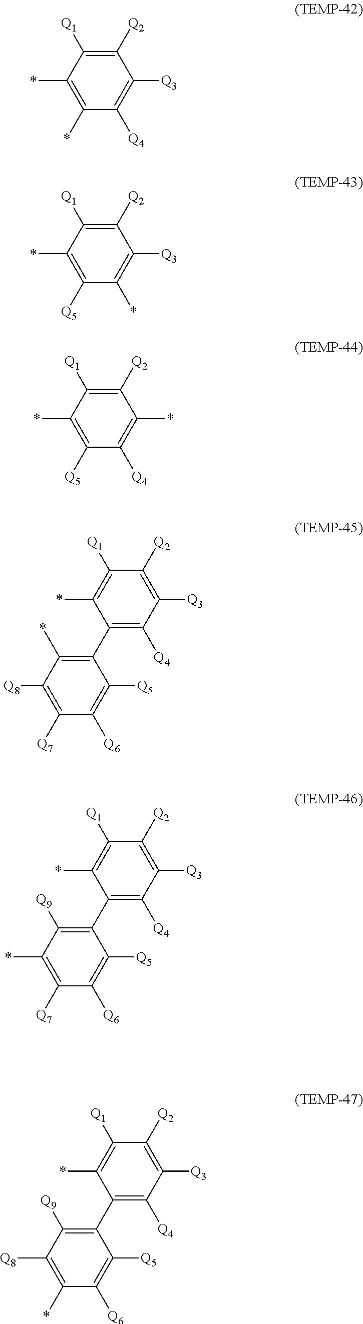

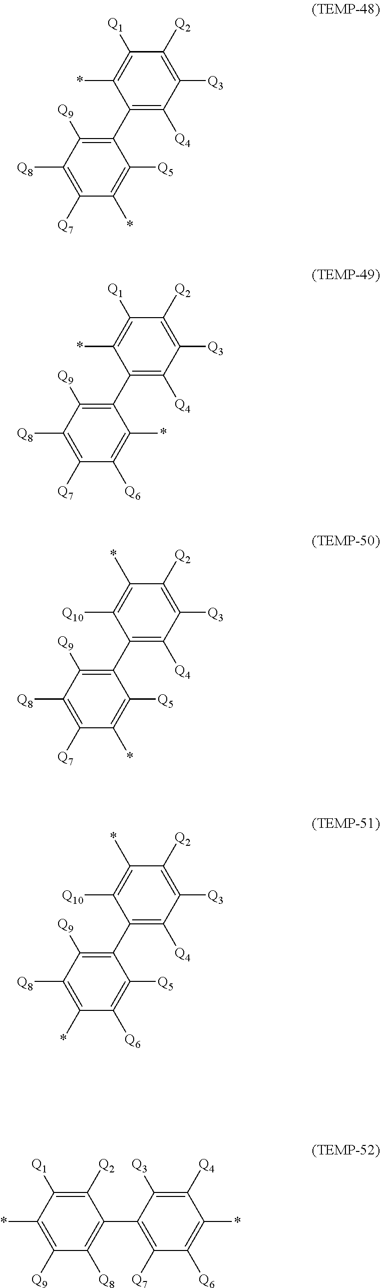

In the general formulae (TEMP-42) to (TEMP-52), Q₁ to Q₁₀ each independently represent a hydrogen atom or a substituent.

In the general formulae (TEMP-42) to (TEMP-52), * represents a bonding site.

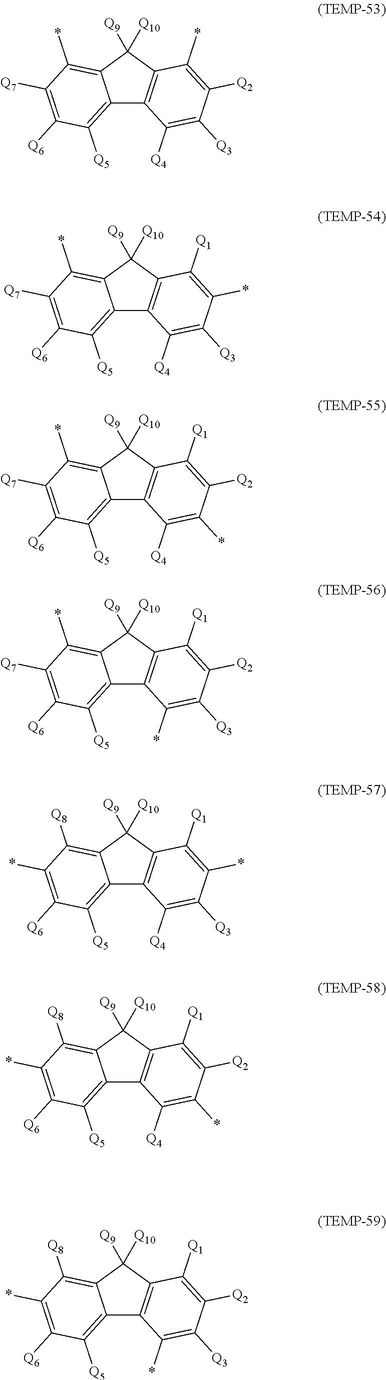

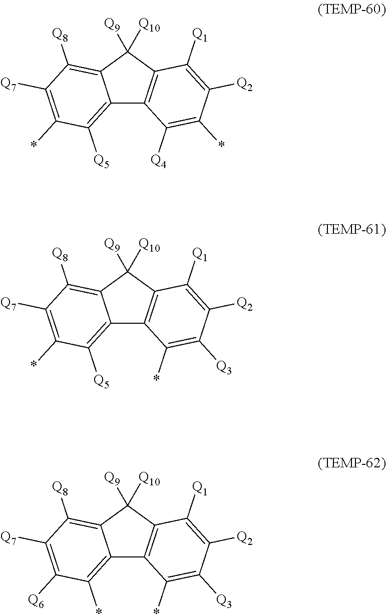

In the general formulae (TEMP-53) to (TEMP-62), Q₁ to Q₁₀ each independently represent a hydrogen atom or a substituent.

The formulae Q₉ and Q₁₀ may be bonded to each other to form a ring via a single bond.

In the general formulae (TEMP-53) to (TEMP-62), * represents a bonding site.

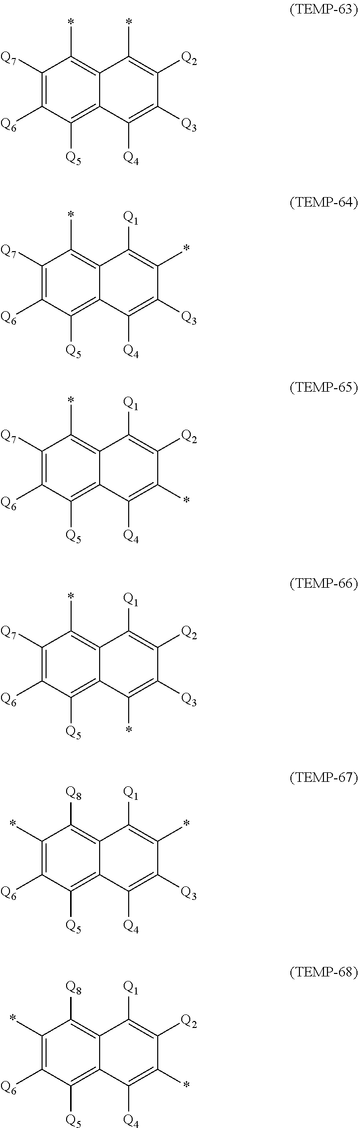

In the general formulae (TEMP-63) to (TEMP-68), Q₁ to Q₈ each independently represent a hydrogen atom or a substituent.

In the general formulae (TEMP-63) to (TEMP-68), * represents a bonding site.

In the description herein, the substituted or unsubstituted divalent heterocyclic group is preferably the groups represented by the following general formulae (TEMP-69) to (TEMP-102) unless otherwise indicated in the description.

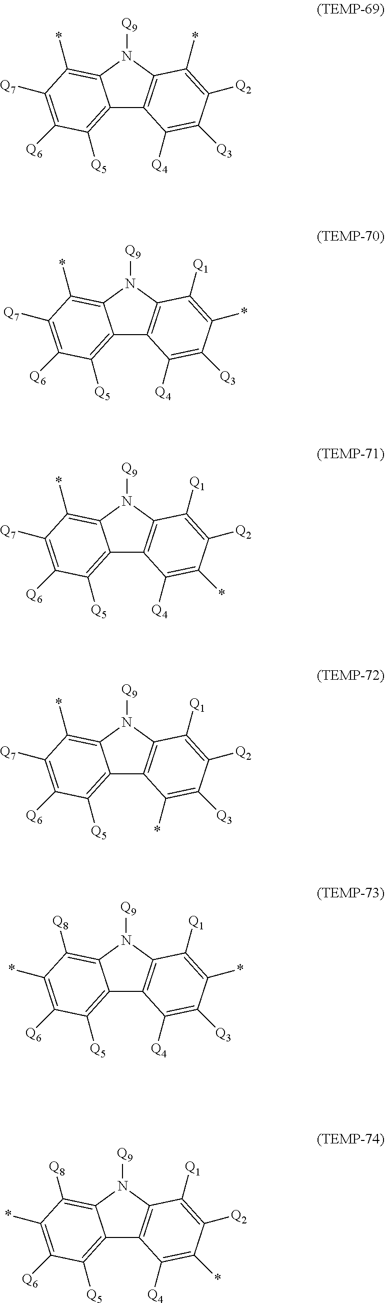

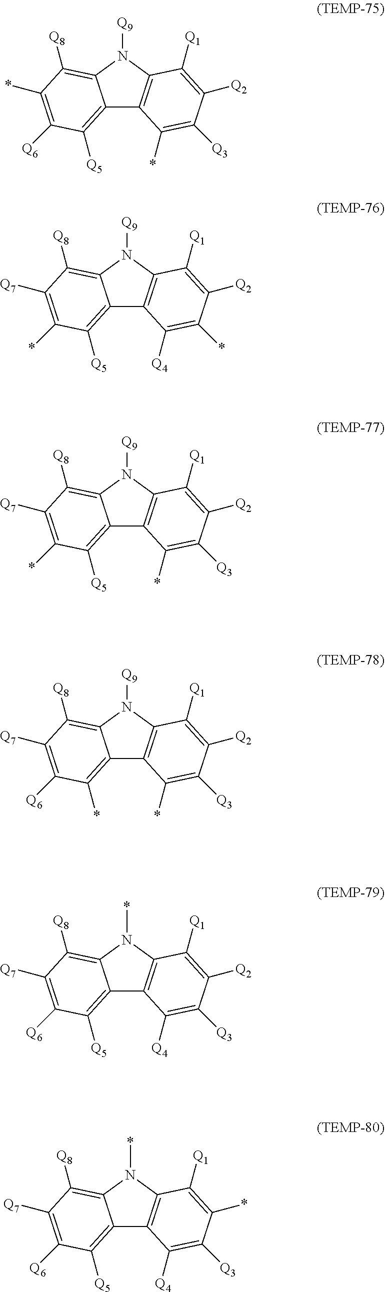

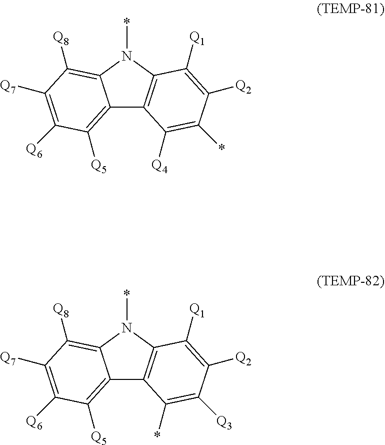

In the general formulae (TEMP-69) to (TEMP-82), Q₁ to Q₉ each independently represent a hydrogen atom or a substituent.

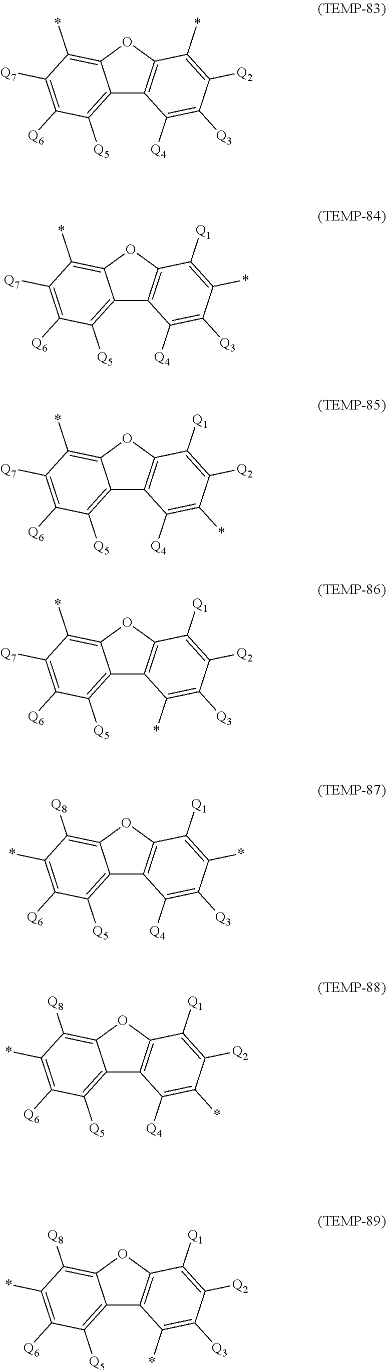

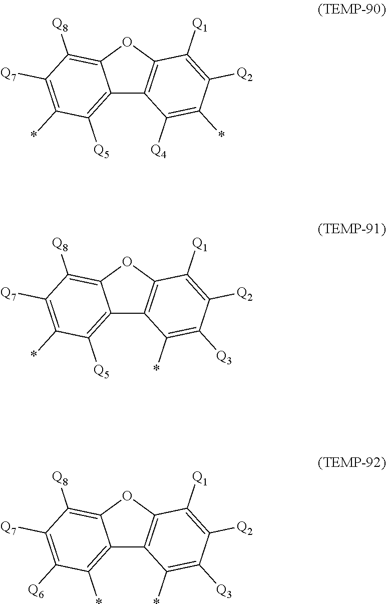

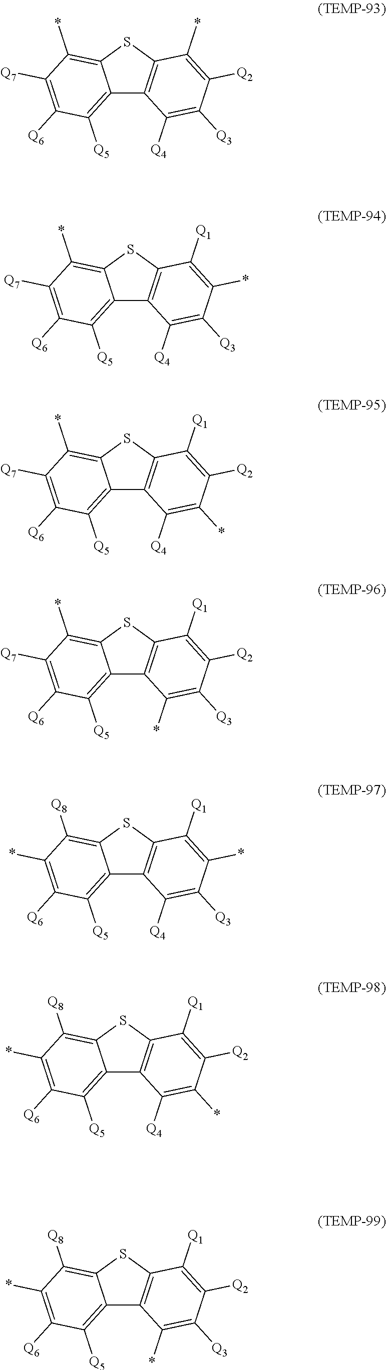

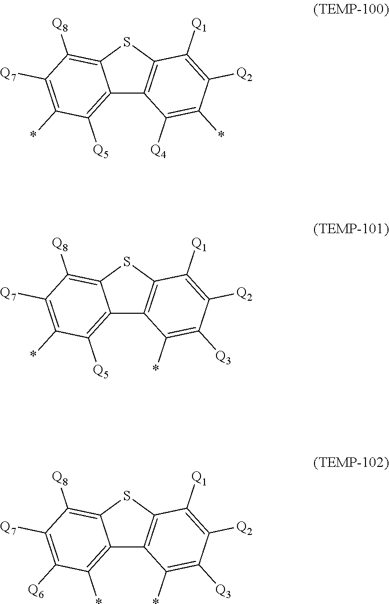

In the general formulae (TEMP-83) to (TEMP-102), Q₁ to Q₈ each independently represent a hydrogen atom or a substituent.

The above are the explanation of the “substituents in the description herein”.

### Case Forming Ring by Bonding

In the description herein, the case where “one or more combinations of combinations each including adjacent two or more each are bonded to each other to form a substituted or unsubstituted monocyclic ring, or each are bonded to each other to form a substituted or unsubstituted condensed ring, or each are not bonded to each other” means a case where “one or more combinations of combinations each including adjacent two or more each are bonded to each other to form a substituted or unsubstituted monocyclic ring”, a case where “one or more combinations of combinations each including adjacent two or more each are bonded to each other to form a substituted or unsubstituted condensed ring”, and a case where “one or more combinations of combinations each including adjacent two or more each are not bonded to each other”.

In the description herein, the case where “one or more combinations of combinations each including adjacent two or more each are bonded to each other to form a substituted or unsubstituted monocyclic ring” and the case where “one or more combinations of combinations each including adjacent two or more each are bonded to each other to form a substituted or unsubstituted condensed ring” (which may be hereinafter collectively referred to as a “case forming a ring by bonding”) will be explained below. The cases will be explained for the anthracene compound represented by the following general formula (TEMP-103) having an anthracene core skeleton as an example.

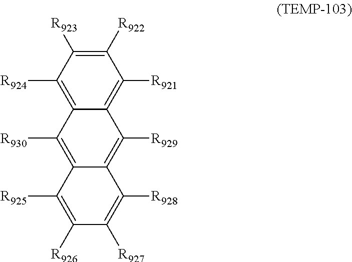

For example, in the case where “one or more combinations of combinations each including adjacent two or more each are bonded to each other to form a ring” among R₉₂₁ to R₉₃₀, the combinations each including adjacent two as one combination include a combination of R₉₂₁ and R₉₂₂, a combination of R₉₂₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₆, a combination of R₉₂₆ and R₉₂₇, a combination of R₉₂₇ and R₉₂₈, a combination of R₉₂₈ and R₉₂₉, and a combination of R₉₂₉ and R₉₂₁.

The “one or more combinations” mean that two or more combinations each including adjacent two or more may form rings simultaneously. For example, in the case where R₉₂₁ and R₉₂₂ are bonded to each other to form a ring Q_{A}, and simultaneously R₉₂₅ and R₉₂₆ are bonded to each other to form a ring Q_{B}, the anthracene compound represented by the general formula (TEMP-103) is represented by the following general formula (TEMP-104).

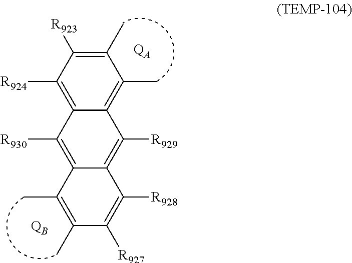

The case where the “combination including adjacent two or more forms rings” encompasses not only the case where adjacent two included in the combination are bonded as in the aforementioned example, but also the case where adjacent three or more included in the combination are bonded. For example, this case means that R₉₂₁ and R₉₂₂ are bonded to each other to form a ring Q_{A}, R₉₂₂ and R₉₂₃ are bonded to each other to form a ring Q_{C}, and adjacent three (R₉₂₁, R₉₂₂, and R₉₂₃) included in the combination are bonded to each other to form rings, which are condensed to the anthracene core skeleton, and in this case, the anthracene compound represented by the general formula (TEMP-103) is represented by the following general formula (TEMP-105). In the following general formula (TEMP-105), the ring Q_{A} and the ring Q_{C} share R₉₂₂.

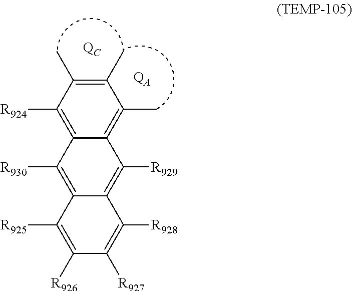

The formed “monocyclic ring” or “condensed ring” may be a saturated ring or an unsaturated ring in terms of structure of the formed ring itself. In the case where the “one combination including adjacent two” forms a “monocyclic ring” or a “condensed ring”, the “monocyclic ring” or the “condensed ring” may form a saturated ring or an unsaturated ring. For example, the ring Q_{A} and the ring Q_{B} formed in the general formula (TEMP-104) each are a “monocyclic ring” or a “condensed ring”. The ring Q_{A} and the ring Q_{C} formed in the general formula (TEMP-105) each are a “condensed ring”. The ring Q_{A} and the ring Q_{C} in the general formula (TEMP-105) form a condensed ring through condensation of the ring Q_{A} and the ring Q_{C}. In the case where the ring Q_{A} in the general formula (TMEP-104) is a benzene ring, the ring Q_{A} is a monocyclic ring. In the case where the ring Q_{A} in the general formula (TMEP-104) is a naphthalene ring, the ring Q_{A} is a condensed ring.

The “unsaturated ring” means an aromatic hydrocarbon ring or an aromatic heterocyclic ring. The “saturated ring” means an aliphatic hydrocarbon ring or anon-aromatic heterocyclic ring.

Specific examples of the aromatic hydrocarbon ring include the structures formed by terminating the groups exemplified as the specific examples in the set of specific examples G1 with a hydrogen atom.

Specific examples of the aromatic heterocyclic ring include the structures formed by terminating the aromatic heterocyclic groups exemplified as the specific examples in the set of specific examples G2 with a hydrogen atom.

Specific examples of the aliphatic hydrocarbon ring include the structures formed by terminating the groups exemplified as the specific examples in the set of specific examples G6 with a hydrogen atom.

The expression “to form a ring” means that the ring is formed only with the plural atoms of the core structure or with the plural atoms of the core structure and one or more arbitrary element. For example, the ring Q_{A} formed by bonding R₉₂₁ and R₉₂₂ each other shown in the general formula (TEMP-104) means a ring formed with the carbon atom of the anthracene skeleton bonded to R₉₂₁, the carbon atom of the anthracene skeleton bonded to R₉₂₂, and one or more arbitrary element. As a specific example, in the case where the ring Q_{A} is formed with R₉₂₁ and R₉₂₂, and in the case where a monocyclic unsaturated ring is formed with the carbon atom of the anthracene skeleton bonded to R₉₂₁, the carbon atom of the anthracene skeleton bonded to R₉₂₂, and four carbon atoms, the ring formed with R₉₂₁ and R₉₂₂ is a benzene ring.

Herein, the “arbitrary element” is preferably at least one kind of an element selected from the group consisting of a carbon element, a nitrogen element, an oxygen element, and a sulfur element, unless otherwise indicated in the description. For the arbitrary element (for example, for a carbon element or a nitrogen element), a bond that does not form a ring may be terminated with a hydrogen atom or the like, and may be substituted by an “arbitrary substituent” described later. In the case where an arbitrary element other than a carbon element is contained, the formed ring is a heterocyclic ring.

The number of the “one or more arbitrary element” constituting the monocyclic ring or the condensed ring is preferably 2 or more and 15 or less, more preferably 3 or more and 12 or less, and further preferably 3 or more and 5 or less, unless otherwise indicated in the description.

What is preferred between the “monocyclic ring” and the “condensed ring” is the “monocyclic ring” unless otherwise indicated in the description.

What is preferred between the “saturated ring” and the “unsaturated ring” is the “unsaturated ring” unless otherwise indicated in the description.

The “monocyclic ring” is preferably a benzene ring unless otherwise indicated in the description.

The “unsaturated ring” is preferably a benzene ring unless otherwise indicated in the description.

In the case where the “one or more combinations of combinations each including adjacent two or more” each are “bonded to each other to form a substituted or unsubstituted monocyclic ring”, or each are “bonded to each other to form a substituted or unsubstituted condensed ring”, it is preferred that the one or more combinations of combinations each including adjacent two or more each are bonded to each other to form a substituted or unsubstituted “unsaturated ring” containing the plural atoms of the core skeleton and 1 or more and 15 or less at least one kind of an element selected from the group consisting of a carbon element, a nitrogen element, an oxygen element, and a sulfur element, unless otherwise indicated in the description.

In the case where the “monocyclic ring” or the “condensed ring” has a substituent, the substituent is, for example, an “arbitrary substituent” described later. In the case where the “monocyclic ring” or the “condensed ring” has a substituent, specific examples of the substituent include the substituents explained in the section “Substituents in Description” described above.

In the case where the “saturated ring” or the “unsaturated ring” has a substituent, the substituent is, for example, an “arbitrary substituent” described later. In the case where the “monocyclic ring” or the “condensed ring” has a substituent, specific examples of the substituent include the substituents explained in the section “Substituents in Description” described above.

The above are the explanation of the case where “one or more combinations of combinations each including adjacent two or more” each are “bonded to each other to form a substituted or unsubstituted monocyclic ring”, and the case where “one or more combinations of combinations each including adjacent two or more” each are “bonded to each other to form a substituted or unsubstituted condensed ring” (i.e., the “case forming a ring by bonding”).

### Substituent for “Substituted or Unsubstituted”

In one embodiment in the description herein, the substituent for the case of “substituted or unsubstituted” (which may be hereinafter referred to as an “arbitrary substituent”) is, for example, a group selected from the group consisting of
an unsubstituted alkyl group having 1 to 50 carbon atoms,
an unsubstituted alkenyl group having 2 to 50 carbon atoms,
an unsubstituted alkynyl group having 2 to 50 carbon atoms,
an unsubstituted cycloalkyl group having 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
an unsubstituted aryl group having 6 to 50 ring carbon atoms, and
an unsubstituted heterocyclic group having 5 to 50 ring atoms,
wherein R₉₀₁ to R₉₀₇ each independently represent
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or a substituted or unsubstituted heterocyclic group having 5 to 50 ring atoms.

In the case where two or more groups each represented by R₉₀₁ exist, the two or more groups each represented by R₉₀₁ are the same as or different from each other,
in the case where two or more groups each represented by R₉₀₂ exist, the two or more groups each represented by R₉₀₂ are the same as or different from each other,
in the case where two or more groups each represented by R₉₀₃ exist, the two or more groups each represented by R₉₀₃ are the same as or different from each other,
in the case where two or more groups each represented by R₉₀₄ exist, the two or more groups each represented by R₉₀₄ are the same as or different from each other,
in the case where two or more groups each represented by R₉₀₅ exist, the two or more groups each represented by R₉₀₅ are the same as or different from each other,
in the case where two or more groups each represented by R₉₀₆ exist, the two or more groups each represented by R₉₀₆ are the same as or different from each other, and
in the case where two or more groups each represented by R₉₀₇ exist, the two or more groups each represented by R₉₀₇ are the same as or different from each other.

In one embodiment, the substituent for the case of “substituted or unsubstituted” may be a group selected from the group consisting of
an alkyl group having 1 to 50 carbon atoms,
an aryl group having 6 to 50 ring carbon atoms, and
a heterocyclic group having 5 to 50 ring atoms.

In one embodiment, the substituent for the case of “substituted or unsubstituted” may be a group selected from the group consisting of
an alkyl group having 1 to 18 carbon atoms,
an aryl group having 6 to 18 ring carbon atoms, and
a heterocyclic group having 5 to 18 ring atoms.

The specific examples of the groups for the arbitrary substituent described above are the specific examples of the substituent described in the section “Substituents in Description” described above.

In the description herein, the arbitrary adjacent substituents may form a “saturated ring” or an “unsaturated ring”, preferably form a substituted or unsubstituted saturated 5-membered ring, a substituted or unsubstituted saturated 6-membered ring, a substituted or unsubstituted unsaturated 5-membered ring, or a substituted or unsubstituted unsaturated 6-membered ring, and more preferably form a benzene ring, unless otherwise indicated.

In the description herein, the arbitrary substituent may further have a substituent unless otherwise indicated in the description. The definition of the substituent that the arbitrary substituent further has may be the same as the arbitrary substituent.

In the description herein, a numerical range shown by “AA to BB” means a range including the numerical value AA as the former of “AA to BB” as the lower limit value and the numerical value BB as the latter of “AA to BB” as the upper limit value.

Hereinafter, a compound of the present invention will be described.

A compound according to one embodiment of the present invention is represented by the following formula (1).

However, hereinafter, the compound of the present invention represented by formula (1) and each formula included in formula (1) described later may be simply referred to as “compound (1),” “inventive compound (1),” or the “inventive compound.”

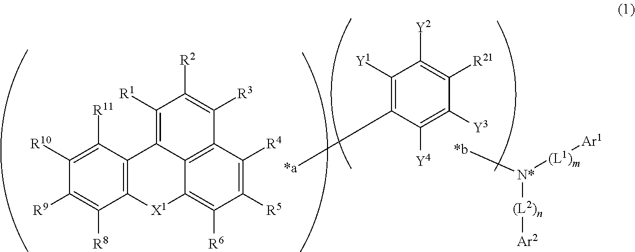

Hereinafter, symbols in formula (1) and each formula included in formula (1) described later will be described. Note that the same symbols have the same meanings. In addition, as shown below, in formula (1), a partial structure bonded to *a may be referred to as a “partial structure A,” a partial structure bonded to *b may be referred to as a “partial structure B,” a partial structure represented by “-(L¹)ₘ-Ar¹” may be referred to as a “partial structure C1,” and a partial structure represented by “-(L²)ₙ-Ar²” may be referred to as a “partial structure C2,” in the present specification.

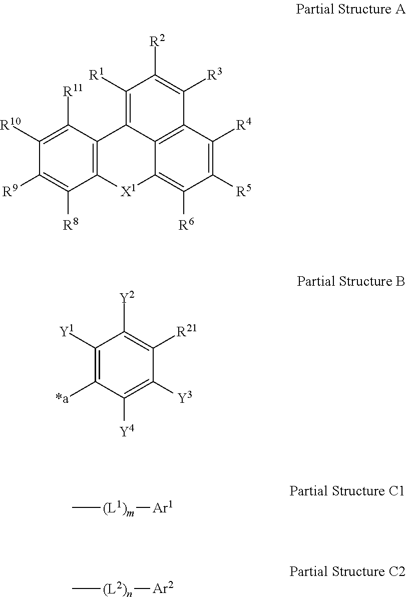

In formula (1), X¹ is an oxygen atom or a sulfur atom, preferably an oxygen atom.

In formula (1), one selected from R¹ to R⁶ and R⁸ to R¹¹ is a single bond bonded to *a, and it is preferable that one selected from R², R⁴, and R¹⁰ be a single bond bonded to *a.

In other words, the partial structure A in the formula (1) is represented by any one of the following formulae (1x-1) to (1x-10) and is preferably represented by any one of the formulae (1x-2), (1x-4), and (1x-10).

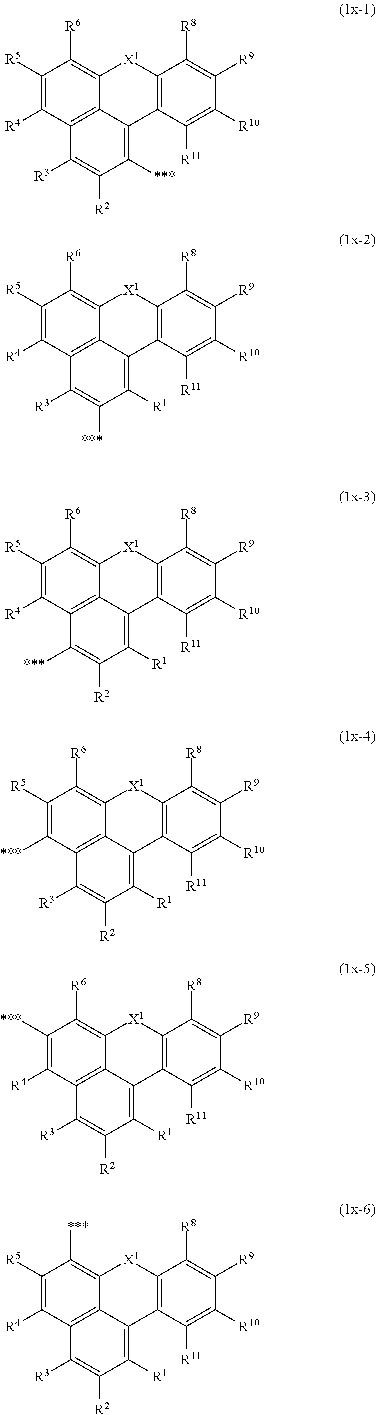

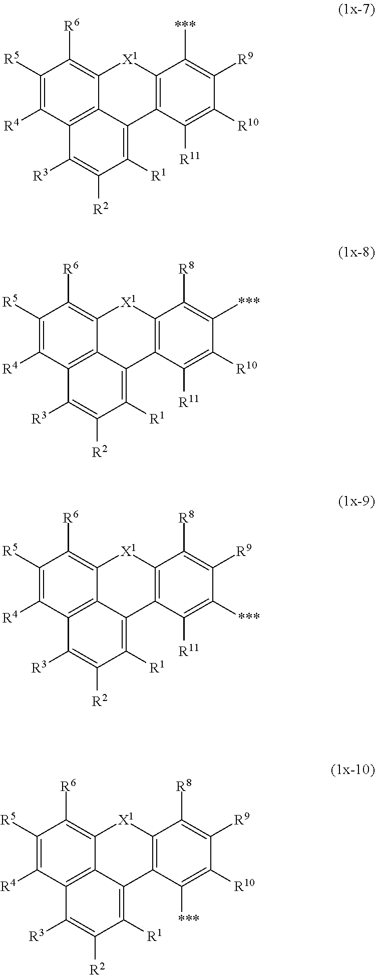

In formulae (1x-1) to (1x-10), *** represents a bonding site to *a. R¹ to R⁶ and R⁸ to R¹¹ are as defined in relation to the formula (1).

R¹ to R⁶ and R⁸ to R¹¹ that are not the single bond are each independently a hydrogen atom, an unsubstituted alkyl group having 1 to 30 carbon atoms, or an unsubstituted aryl group having 6 to 12 ring carbon atoms, preferably each independently a hydrogen atom, a substituted or unsubstituted alkyl group having 1 to 6 carbon atoms, or an aryl group having 6 to 12 ring carbon atoms, and more preferably a hydrogen atom.

All of R¹ to R⁶ and R⁸ to R¹¹ that are not the single bond may be hydrogen atoms.

The unsubstituted alkyl group of the substituted or unsubstituted alkyl group having 1 to 30 carbon atoms is, for example,
a methyl group, an ethyl group, a n-propyl group, an isopropyl group, a n-butyl group, an isobutyl group, a s-butyl group, a t-butyl group, a pentyl group, a hexyl group, a heptyl group, an octyl group, a nonyl group, a decyl group, an undecyl group, or a dodecyl group;
preferably a methyl group, an ethyl group, a n-propyl group, an isopropyl group, a n-butyl group, an isobutyl group, a s-butyl group, a t-butyl group, or a pentyl group;
more preferably a methyl group, an ethyl group, a n-propyl group, an isopropyl group, a n-butyl group, an isobutyl group, a s-butyl group, or a t-butyl group;
still more preferably a methyl group, an ethyl group, an isopropyl group, or a t-butyl group; and
especially preferably a methyl group.

The unsubstituted aryl group of the substituted or unsubstituted aryl group having 6 to 12 ring carbon atoms is, for example,
a phenyl group, a biphenyl group, or a naphthyl group;
preferably a phenyl groups, a 2-, 3- or 4-biphenylyl group, or a 1- or 2-naphthyl group; and
especially preferably a phenyl group.

Each combination of adjacent moieties among R¹ to R⁶ and R⁸ to R¹¹ that are not the single bond forms no ring, with the adjacent moieties not bonded to each other.

In formula (1), R²¹ is a hydrogen atom.

In formula (1), one selected from Y¹ to Y⁴ is a single bond bonded to *b, and it is preferable that one selected from Y² and Y³ be a single bond bonded to *b.

Y¹ to Y⁴ that are not the single bond are hydrogen atoms.

In formula (1), N* is a central nitrogen atom.

In formula (1), m is 0 or 1, and n is 0 or 1.

When m is 0, Ar¹ is directly bonded to the central nitrogen atom *.

When n is 0, Ar² is directly bonded to the central nitrogen atom *.

In one embodiment, m and n are both 0. In another embodiment, m and n are both 1. In yet another aspect, m is 0, and n is 1. In yet another embodiment, m is 1, and n is 0.

In formula (1), L¹ and L² are each independently a substituted or unsubstituted phenylene group, a substituted or unsubstituted naphthylene group, or a substituted or unsubstituted biphenylene group, preferably a substituted or unsubstituted phenylene group, or a substituted or unsubstituted biphenylene group, and more preferably a substituted or unsubstituted phenylene group.

The phenylene group is an o-phenylene group, a m-phenylene group, or a p-phenylene group, and a p-phenylene group is preferable.

The biphenylene group is a 4,2′-biphenylene group, a 4,3′-biphenylene group, a 4,4′-biphenylene group, a 3,2′-biphenylene group, a 3,3′-biphenylene group, or a 2,2′-biphenylene group, preferably a 4,2′-biphenylene group, a 4,3′-biphenylene group, a 4,4′-biphenylene group, or a 3,3′-biphenylene group, and more preferably a 4,4′-biphenylene group.

The naphthylene group is preferably a 1,4-naphthylene group, a 2,6-naphthylene group, a 1,5-naphthylene group, or a 1,8-naphthylene group.

A substituent of the phenylene group, the naphthylene group, and the biphenylene group that L¹ and L² may take is selected from an unsubstituted alkyl group having 1 to 6 carbon atoms and an aryl group having 6 to 12 ring carbon atoms, and substituents are not bonded to each other and form no ring.

The details of the unsubstituted alkyl group having 1 to 6 carbon atoms as the substituent are as described for R¹ to R⁶ and R⁸ to R¹⁰ of formula (1) except that the number of carbon atoms is 1 to 6.

The details of the unsubstituted aryl group having 6 to 12 ring carbon atoms as the substituent are as described for R¹ to R⁶ and R⁸ to R¹⁰ of formula (1).

When L¹ and L² are present in formula (1), L¹ and L² may be the same as or different from each other. Only one of L¹ and L² may be present (that is, one of m and n may be 0 and the other may be 1), or both L¹ and L² may not be present (that is, m and n may be 0).

In other words, the combination of “-(L¹)ₘ-” and “-(L²)ₙ-” in compound (1) is represented by any of the following combinations [k1] to [k10].
[k1]: single bond/single bond
[k2]: single bond/phenylene
[k3]: single bond/naphthylene
[k4]: single bond/biphenylene
[k5]: phenylene/phenylene
[k6]: phenylene/naphthylene
[k7]: phenylene/biphenylene
[k8]: naphthylene/naphthylene
[k9]: naphthylene/biphenylene
[k10]: biphenylene/biphenylene

Among them, [k1], [k2], [k4], [k5], [k7], and [k10] are preferable.

In one embodiment, in the formula (1), one of m and n is 1, and the other is 0, and the compound is represented by, for example, the following formula (11).

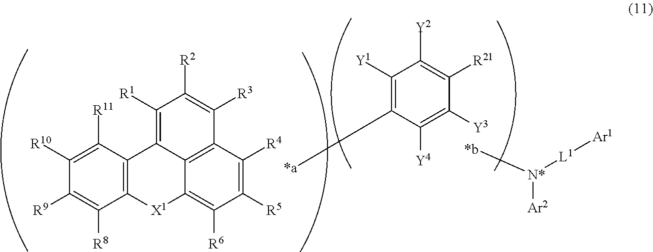

In formula (11), X¹, R¹ to R⁶, R⁸ to R¹¹, R²¹, Y¹ to Y⁴, N*, L¹, Ar¹, Ar², *a, and *b are as defined for the formula (1).

In one embodiment, in the formula (1), m and n are 1, and the compound is represented by the following formula (12).

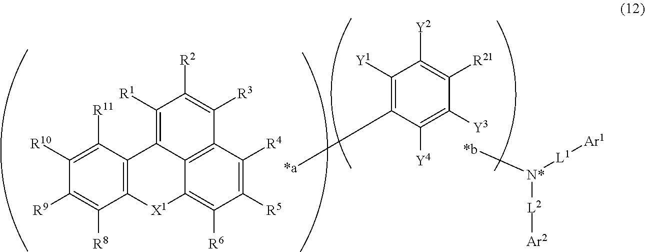

In formula (12), X¹, R¹ to R⁶, R⁸ to R¹¹, R²¹, Y¹ to Y⁴, N*, L¹, L², Ar¹, Ar², *a, and *b are as defined for the formula (1).

In one embodiment, in the formula (1), m and n are 0, and the compound is represented by the following formula (13).

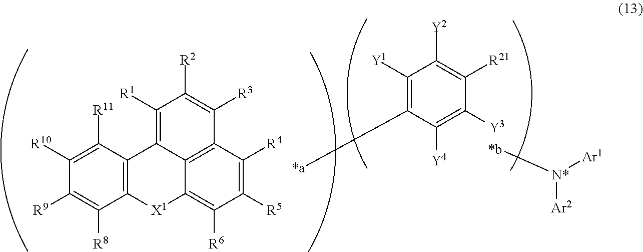

In formula (13), X¹, R¹ to R⁶, R⁸ to R¹¹, R²¹, Y¹ to Y⁴, N*, Ar¹, Ar², *a, and *b are as defined for the formula (1).

In formula (1), Ar¹ and Ar² are each independently represented by any of the following formulae (1-a) to (1-d).

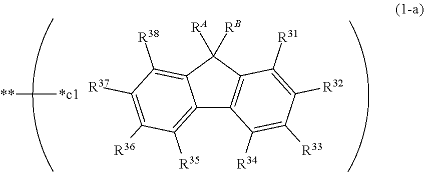

In formula (1-a), ** represents a bonding site to one or both of L¹ and L².

In formula (1-a), one selected from R³¹ to R³⁸, R^{A}, and R^{B} is a single bond bonded to *c1, or one selected from R^{A} and R^{B} is a divalent group bonded to *c1. It is preferable that one selected from R³², R³⁴, R³, R³⁷, R^{A}, and R^{B} be a single bond bonded to *c1, or one selected from R^{A} and R^{B} be a divalent group bonded to *c1.

When R^{A} is the group bonded to *c1, m is preferably 0.

When R^{B} is the group bonded to *c1, n is preferably 0.

When Ar¹ is represented by the formula (1-a) and one of R³¹ to R³⁸ in the formula (1-a) is a single bond bonded to *c1, m is preferably 0.

When Ar² is represented by the formula (1-a) and one of R³¹ to R³⁸ in the formula (1-a) is a single bond bonded to *c1, n is preferably 0.

R³¹ to R³⁸ that are not the single bond are each independently a hydrogen atom, an unsubstituted alkyl group having 1 to 30 carbon atoms, or an unsubstituted aryl group having 6 to 12 ring carbon atoms, preferably each independently a hydrogen atom, a substituted or unsubstituted alkyl group having 1 to 6 carbon atoms, or an aryl group having 6 to 12 ring carbon atoms, and more preferably a hydrogen atom.

All of R³¹ to R³⁸ that are not the single bond may be hydrogen atoms.

The details of the substituted or unsubstituted alkyl group having 1 to 30 carbon atoms are as described for R¹ to R⁶ and R⁸ to R¹⁰ of formula (1).

The details of the substituted or unsubstituted aryl group having 1 to 12 ring carbon atoms are as described for R¹ to R⁶ and R⁸ to R¹⁰ of formula (1).

One combination of adjacent moieties among R³¹ to R³⁸ that are not the single bond forms an unsubstituted benzene ring, with the adjacent moieties bonded to each other, or each combination of adjacent moieties among R³¹ to R³⁸ forms no ring, with the adjacent moieties not bonded to each other.

R^{A} and R^{B} that are not the single bond and are not the group bonded to *c1 are a substituted or unsubstituted alkyl group having 1 to 30 carbon atoms, a substituted or unsubstituted aryl group having 6 to 30 ring carbon atoms, or a substituted or unsubstituted heteroaryl group (aromatic heterocyclic group) having 5 to 30 ring atoms, and at least one of R^{A} and R^{B} is a substituted or unsubstituted aryl group having 6 to 30 ring carbon atoms.

The details of the substituted or unsubstituted alkyl group having 1 to 30 carbon atoms are as described for R¹ to R⁶ and R⁸ to R¹⁰ of formula (1).

The unsubstituted aryl group of the substituted or unsubstituted aryl group having 6 to 30, preferably 6 to 18, and more preferably 6 to 12 ring carbon atoms is, for example,
a phenyl group, a biphenyl group, a terphenyl group, a naphthyl group, an anthryl group, a benzanthryl group, a phenanthryl group, a benzophenanthryl group, a pyrenyl group, a chrysenyl group, a benzochrysenyl group, a fluorenyl group, a fluoranthenyl group, a perylenyl group, or a triphenylenyl group;
preferably a phenyl group, a biphenyl group, a terphenyl group, or a naphthyl group;
more preferably a phenyl group, a 2-, 3-, or 4-biphenylyl group, a 2-, 3-, or 4-o-terphenylyl group, a 2-, 3-, or 4-m-terphenylyl group, a 2-, 3-, or 4-p-terphenylyl group, or a 1- or 2-naphthyl group;
still more preferably a phenyl group, a 2-, 3-, or 4-biphenylyl group, or a 1- or 2-naphthyl group; and
especially preferably a phenyl group.

The substituted or unsubstituted heteroaryl group having 5 to 30, preferably 5 to 20, and more preferably 5 to 13 ring atoms is, for example,
a pyrrolyl group, a furyl group, a thienyl group, a pyridyl group, an imidazopyridyl group, a pyridazinyl group, a pyrimidinyl group, a pyrazinyl group, a triazinyl group, an imidazolyl group, an oxazolyl group, a thiazolyl group, a pyrazolyl group, an isoxazolyl group, an isothiazolyl group, an oxadiazolyl group, a thiadiazolyl group, a triazolyl group, a tetrazolyl group, an indolyl group, an isoindolyl group, an indolizinyl group, a quinolizinyl group, a quinolyl group, an isoquinolyl group, a cinnolyl group, a phthalazinyl group, a quinazolinyl group, a quinoxalinyl group, a benzimidazolyl group, a benzoxazolyl group, a benzothiazolyl group, an indazolyl group, a benzisoxazolyl group, a benzisothiazolyl group, a phenanthridinyl group, an acridinyl group, a phenanthrolinyl group, a phenazinyl group, a phenothiazinyl group, a phenoxazinyl group, a xanthenyl group, a benzofuranyl group, an isobenzofuranyl group, a naphthobenzofuranyl group, a dibenzofuranyl group, a benzothiophenyl (benzothienyl group, the same applies hereinafter), an isobenzothiophenyl group (isobenzothienyl group, the same applies hereinafter), a naphthobenzothiophenyl group (naphthobenzothienyl group, the same applies hereinafter), a dibenzothiophenyl group (dibenzothienyl group, the same applies hereinafter), or a carbazolyl group (including a 9-carbazolyl group or a 1-, 2-, 3- or 4-carbazolyl group, the same applies hereinafter);
preferably a benzofuranyl group, an isobenzofuranyl group, a naphthobenzofuranyl group, a dibenzofuranyl group, a benzothiophenyl group, an isobenzothiophenyl group, a naphthobenzothiophenyl group, a dibenzothiophenyl group, or a carbazolyl group; and
more preferably a dibenzofuranyl group, a dibenzothiophenyl group, or a carbazolyl group.

The divalent group bonded to *c1 represented by R^{A} and R^{B} is a substituted or unsubstituted alkylene group having 1 to 30 carbon atoms, a substituted or unsubstituted arylene group having 6 to 30 ring carbon atoms, or a substituted or unsubstituted heteroarylene group having 5 to 30 ring atoms.

Examples of the alkylene group, the arylene group, and the heteroarylene group having 5 to 30 atoms represented by R^{A} and R^{B} include divalent residues of the respective groups described with reference to the alkyl group, the aryl group, and the heteroaryl group represented by R^{A} and R^{B}, and preferable examples thereof are also the same.

R^{A} and R^{B} that are not the single bond and are not the group bonded to *c1 are bonded to each other to form a ring or are not bonded to each other and form no ring.

The ring formed by R^{A} and R^{B} that are not the single bond, are not the group bonded to *c1, and are bonded to each other is a substituted or unsubstituted spiro ring. The spiro ring is a hydrocarbon ring or a heterocyclic ring, and is selected from a monocyclic ring, a condensed ring, a bridged bicyclo ring, and a bridged tricyclo ring. Examples of the substituted or unsubstituted spiro ring are shown below, but are not limited thereto. The symbol * represents a bonding position to a benzene ring of the fluorene skeleton.

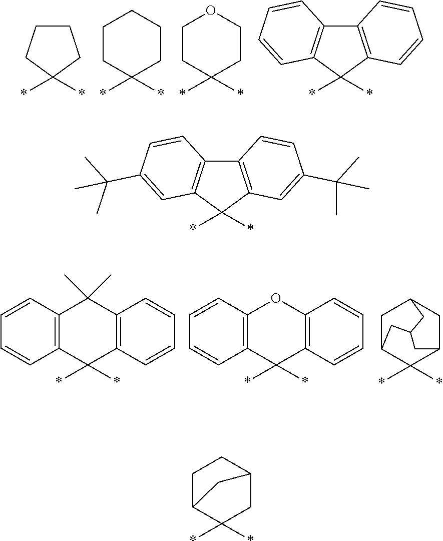

In one embodiment, Ar¹ or Ar² represented by the formula (1-a) is represented by any of the following formulae (1-a1) to (1-a5).

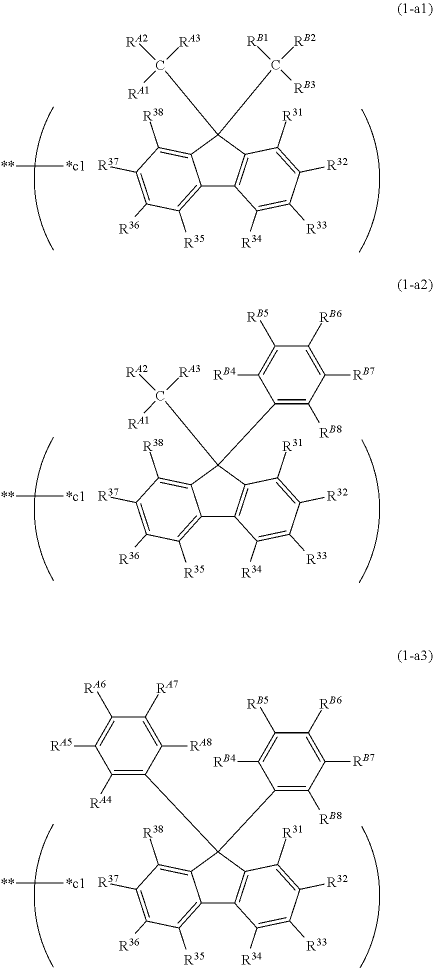

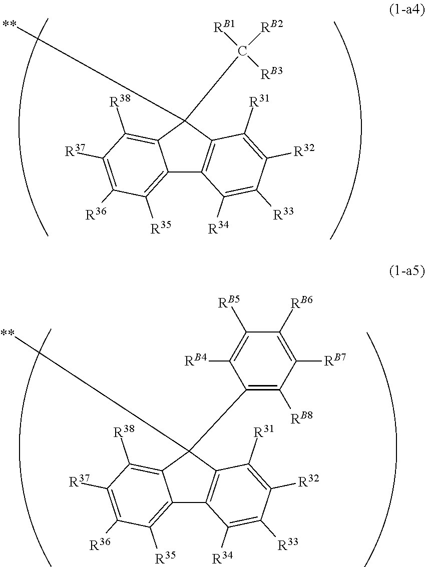

In formulae (1-a1) to (1-a5), R³¹ to R³⁸, **, and *c1 are as defined for the formula (1).

In formulae (1-a1) to (1-a5), R^{A1} to R^{A3}, R^{A4} to R^{A8}, R^{B1} to R^{B3}, and R^{B4} to R^{B8} are hydrogen atoms.

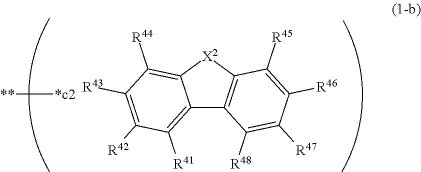

In formula (1-b), ** represents a bonding site to one or both of L¹ and L².

In formula (1-b), X² is an oxygen atom or a sulfur atom and is preferably an oxygen atom.

In formula (1-b), one selected from R⁴¹ to R⁴⁸ is a single bond bonded to *c2.

R⁴¹ to R⁴⁸ that are not the single bond are each independently a hydrogen atom, an unsubstituted alkyl group having 1 to 30 carbon atoms, or an unsubstituted aryl group having 6 to 12 ring carbon atoms, preferably each independently a hydrogen atom, a substituted or unsubstituted alkyl group having 1 to 6 carbon atoms, or an aryl group having 6 to 12 ring carbon atoms, and more preferably a hydrogen atom.

All of R⁴¹ to R⁴⁸ that are not the single bond may be hydrogen atoms.

The details of the substituted or unsubstituted alkyl group having 1 to 30 carbon atoms are as described for R¹ to R⁶ and R⁸ to R¹⁰ of formula (1).

The details of the substituted or unsubstituted aryl group having 1 to 12 ring carbon atoms are as described for R¹ to R⁶ and R⁸ to R¹⁰ of formula (1).

One combination of adjacent moieties among R⁴¹ to R⁴⁸ that are not the single bond forms an unsubstituted benzene ring, with the adjacent moieties bonded to each other, or each combination of adjacent moieties among R⁴¹ to R⁴⁸ forms no ring, with the adjacent moieties not bonded to each other.

When one combination of adjacent moieties among R⁴¹ to R⁴⁴ that are not the single bond forms an unsubstituted benzene ring, with the adjacent moieties bonded to each other, one selected from R⁴⁵ to R⁴⁸ is preferably a single bond bonded to *c2.

When one combination of adjacent moieties among R⁴⁵ to R⁴⁸ that are not the single bond forms an unsubstituted benzene ring, with the adjacent moieties bonded to each other, one selected from R⁴¹ to R⁴⁴ is preferably a single bond bonded to *c2.

In one embodiment, the formula (1-b) is represented by any one of the following formulae (1-b1) to (1-b3), and is preferably represented by formula (1-b1).

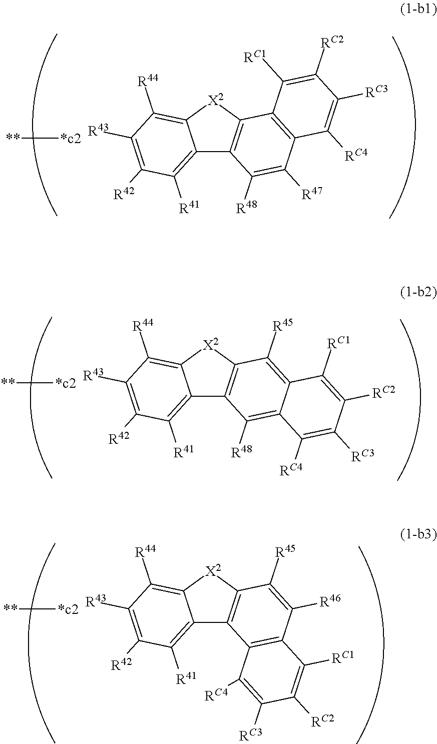

In formulae (1-b1) to (1-b3), X², R⁴¹ to R⁴⁸, **, and *c2 are as defined for the formula (1).

In formulae (1-b1) to (1-b3), R^{C1} to R^{C4} are hydrogen atoms.

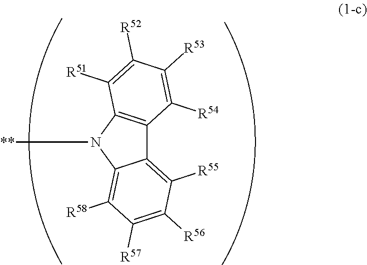

In formula (1-c), ** represents a bonding site to one or both of L¹ and L².

In formula (1-c), R⁵¹ to R⁵⁸ are each independently a hydrogen atom, an unsubstituted alkyl group having 1 to 30 carbon atoms, or an unsubstituted aryl group having 6 to 12 ring carbon atoms, preferably each independently a hydrogen atom, a substituted or unsubstituted alkyl group having 1 to 6 carbon atoms, or an aryl group having 6 to 12 ring carbon atoms, and more preferably a hydrogen atom.

All of R⁵¹ to R⁵⁸ that are not the single bond may be hydrogen atoms.

The details of the substituted or unsubstituted alkyl group having 1 to 30 carbon atoms are as described for R¹ to R⁶ and R⁸ to R¹⁰ of formula (1).

The details of the substituted or unsubstituted aryl group having 1 to 12 ring carbon atoms are as described for R¹ to R⁶ and R⁸ to R¹⁰ of formula (1).

One combination of adjacent moieties among R⁵¹ to R⁵⁸ that are not the single bond forms an unsubstituted benzene ring, with the adjacent moieties bonded to each other, or each combination of adjacent moieties among R⁵¹ to R⁵⁸ forms no ring, with the adjacent moieties not bonded to each other.

When Ar¹ is represented by the formula (1-c), m is preferably 1.

When Ar² is represented by the formula (1-c), n is preferably 1.

**—Z (1-d)

In formula (1-d), ** represents a bonding site to one or both of L¹ and L².

In formula (1-d), Z is a hydrogen atom or a substituted or unsubstituted aryl group consisting only of a 6-membered ring and having 6 to 30 ring carbon atoms, preferably a substituted or unsubstituted aryl group consisting only of a 6-membered ring and having 6 to 24 ring carbon atoms, and more preferably a substituted or unsubstituted aryl group consisting only of a 6-membered ring and having 6 to 18 ring carbon atoms.

Substituents on the aryl group represented by Z are bonded to each other to form a substituted or unsubstituted monocyclic ring, are bonded to each other to form a condensed ring, or are not bonded to each other and form no ring.

The monocyclic ring is preferably a monocyclic ring having 3 or more and 6 or less ring atoms, is a benzene ring, a furan ring, or a thiophene ring, for example, and is preferably a benzene ring.

The condensed ring is a condensed ring formed by the aryl group represented by Z and substituents on the aryl group represented by the Z, and is selected from a substituted or unsubstituted naphthalene ring, a substituted or unsubstituted anthracene ring, a substituted or unsubstituted phenanthrene ring, and a substituted or unsubstituted phenalene ring. The condensed ring is preferably a substituted or unsubstituted naphthalene ring or a substituted or unsubstituted phenanthrene ring, and more preferably a substituted or unsubstituted naphthalene ring.

Each of the monocyclic ring and the condensed ring formed by multiple substituents on the aryl group represented by the Z preferably has no substituent.

When Ar¹ is represented by the formula (1-d), and m is 0, Z in the formula (1-d) is a substituted or unsubstituted aryl group consisting only of a 6-membered ring and having 6 to 30 ring carbon atoms.

When Ar² is represented by the formula (1-d), and n is 0, Z in the formula (1-d) is a substituted or unsubstituted aryl group consisting only of a 6-membered ring and having 6 to 30 ring carbon atoms.

Ar¹ and Ar² may be the same as or different from each other.

In other words, Ar¹ and Ar² in compound (1) is represented by any of combinations shown in the following [a] to [j].
[a]: formula (1-a)/formula (1-a)
[b]: formula (1-a)/formula (1-b)
[c]: formula (1-a)/formula (1-c)
[d]: formula (1-a)/formula (1-d)
[e]: formula (1-b)/formula (1-b)
[f]: formula (1-b)/formula (1-c)
[g]: formula (1-b)/formula (1-d)
[h]: formula (1-c)/formula (1-c)
[i]: formula (1-c)/formula (1-d)
[j]: formula (1-d)/formula (1-d)

Among them, [a] to [g], [i], and [j] are preferable, and [d], [g], [i], and [j] are more preferable.

In one embodiment, compound (1) is represented by any one of the following formulae (1A), (1A1), (1A2), and (1A3).

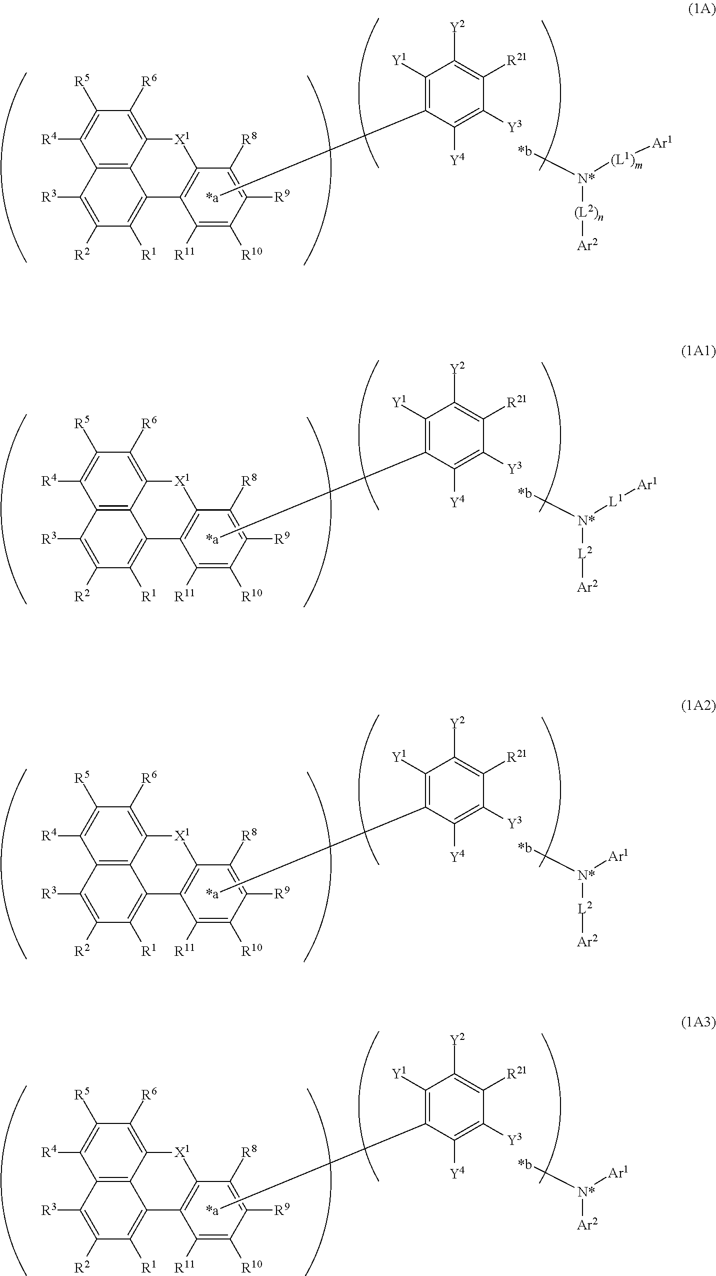

In formulae (1A), (1A1), (1A2), and (1A3), X¹, R¹ to R⁶, R⁸ to R¹¹, R²¹, Y¹ to Y⁴, N*, L¹, L², Ar¹, Ar², *a, *b, m, and n are as defined for the formula (1). Provided that one selected from R⁸ to R¹¹ is a single bond bonded to *a.

In addition, in one embodiment, compound (1) is represented by any one of the following formulae (1), (1B1), (1B2), and (1B3).

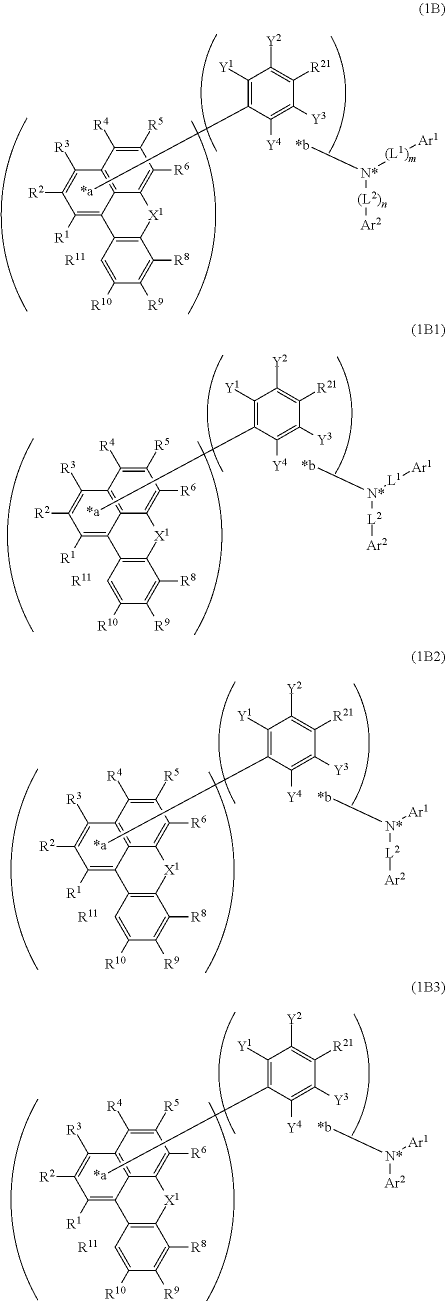

In formulae (1), (1B1), (1B2), and (1B3), X¹, R¹ to R⁶, R⁸ to R¹¹, R²¹, Y¹ to Y⁴, N*, L¹, L², Ar¹, Ar², *a, *b, m, and n are as defined for the formula (1). Provided that one selected from R¹ to R⁶ is a single bond bonded to *a.

In addition, in one embodiment, compound (1) is represented by any one of the following formulae (1C), (1C1), (1C2), and (1C3).

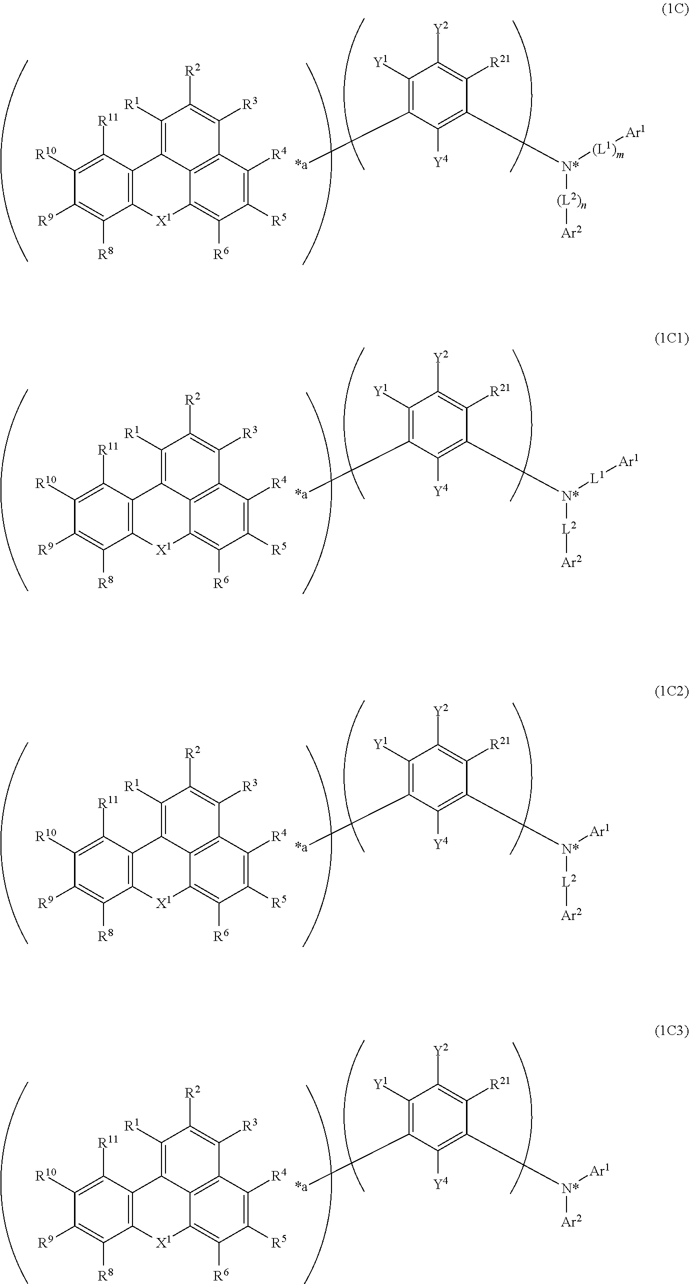

In formulae (1C), (1C1), (1C2), and (1C3), X¹, R¹ to R⁶, R⁸ to R¹¹, R²¹, Y¹, Y², Y⁴, N*, L¹, L², Ar¹, Ar², *a, m, and n are as defined for the formula (1).

In addition, in one embodiment, compound (1) is represented by any one of the following formulae (1D), (1D1), (1D2), and (1D3).

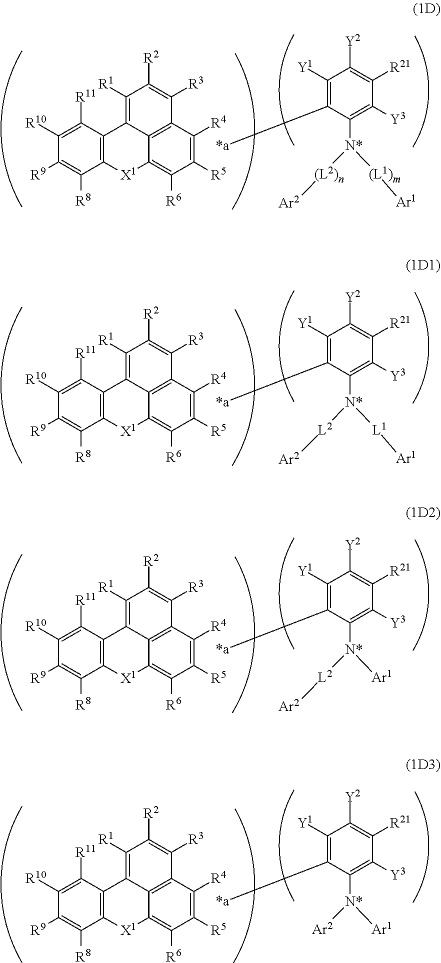

In formulae (1D), (1D1), (1D2), and (1D3), X¹, R¹ to R⁶, R⁸ to R¹¹, R²¹, Y¹ to Y³, N*, L¹, L², Ar¹, Ar², *a, m, and n are as defined for the formula (1).

In addition, in one embodiment, compound (1) is represented by any one of the following formulae (1E), (1E1), (1E2), and (1E3).

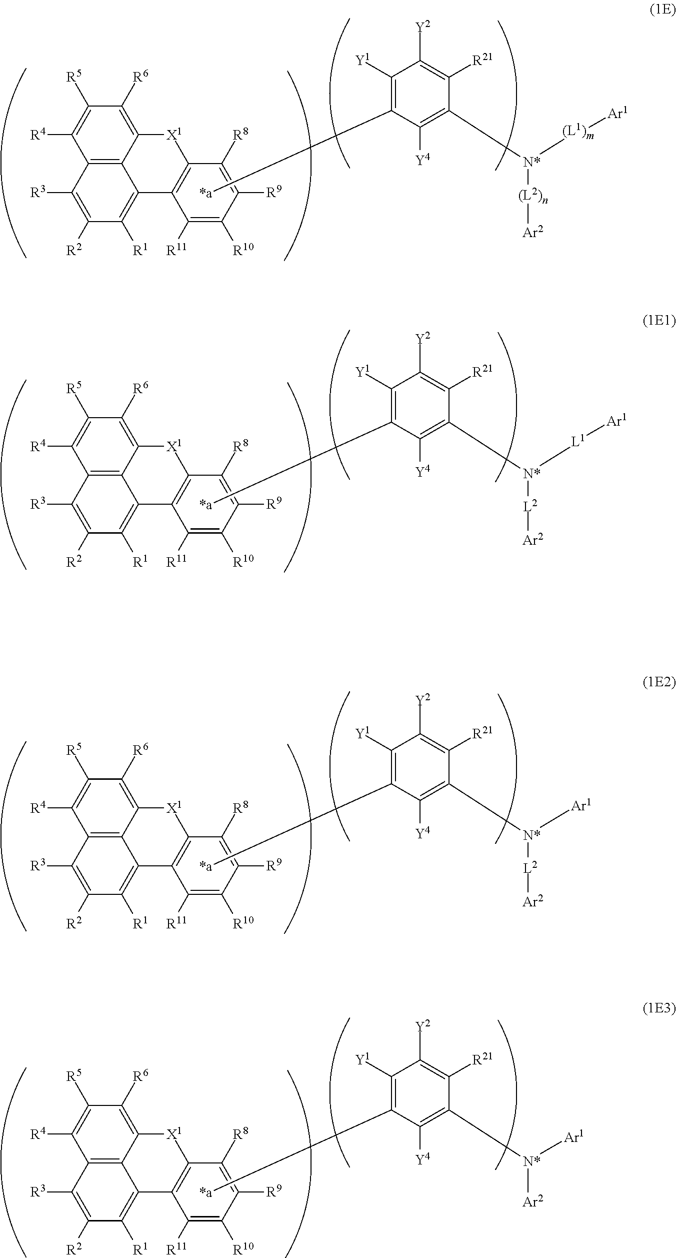

In formulae (1E), (1E1), (1E2), and (1E3), X¹, R¹ to R⁶, R⁸ to R¹¹, R²¹, Y¹, Y², Y⁴, N*, L¹, L², Ar¹, Ar², *a, m, and n are as defined for the formula (1).

In addition, in one embodiment, compound (1) is represented by any one of the following formulae (1F), (1F1), (1F2), and (1F3).

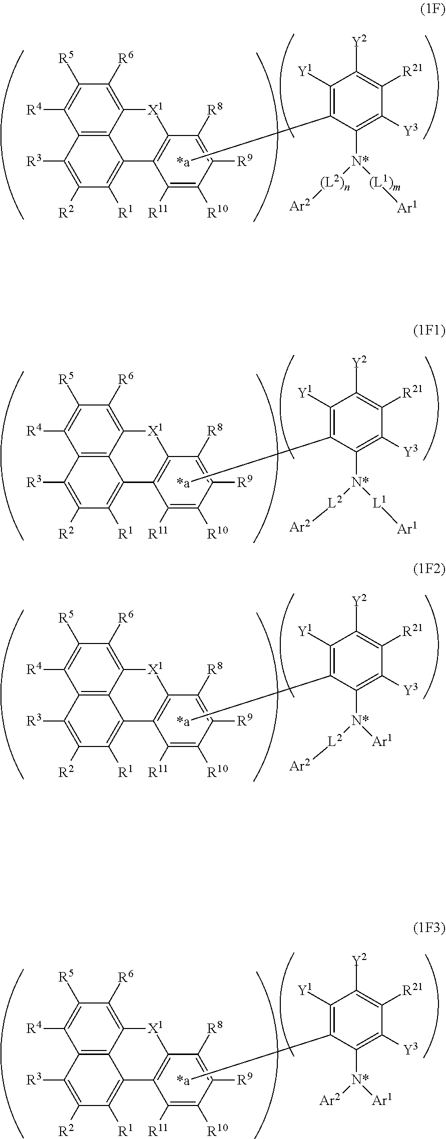

In formulae (1F), (1F1), (1F2), and (1F3), X¹, R¹ to R⁶, R⁸ to R¹¹, R²¹, Y¹ to Y³, N*, L¹, L², Ar¹, Ar², *a, m, and n are as defined for the formula (1).

In addition, in one embodiment, compound (1) is represented by any one of the following formulae (1G), (1G1), (1G2), and (1G3).

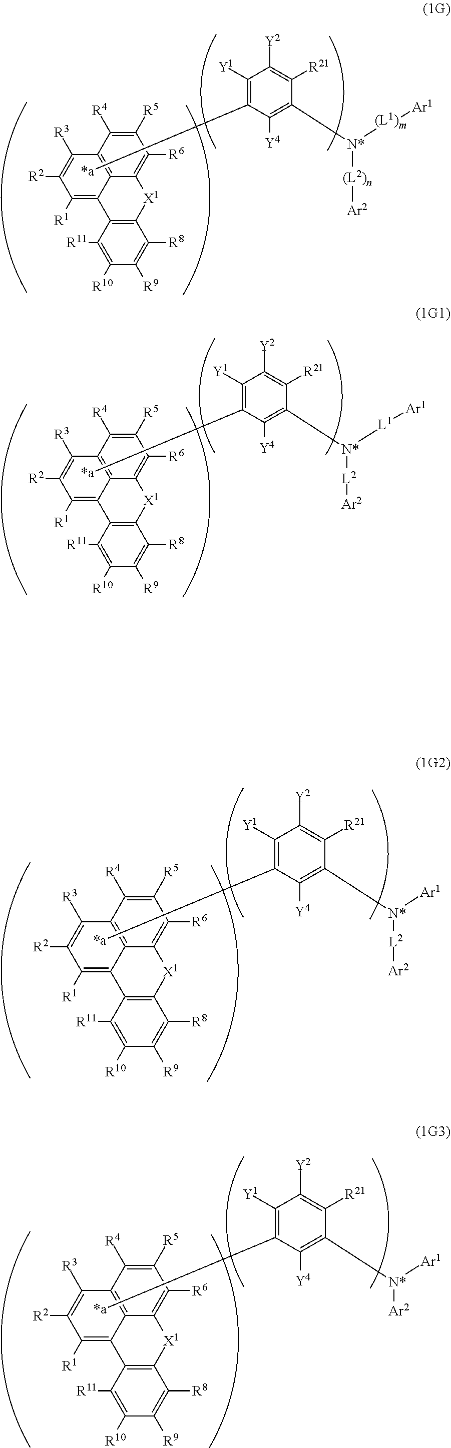

In formulae (1G), (1G1), (1G2), and (1G3), X¹, R¹ to R⁶, R⁸ to R¹¹, R²¹, Y¹, Y², Y⁴, N*, L¹, L², Ar¹, Ar², *a, m, and n are as defined forthe formula (1). Provided that one selected from R¹ to R⁶ is a single bond bonded to *a.

In addition, in one embodiment, compound (1) is represented by any one of the following formulae (1H), (1H1), (1H2), and (1H3).

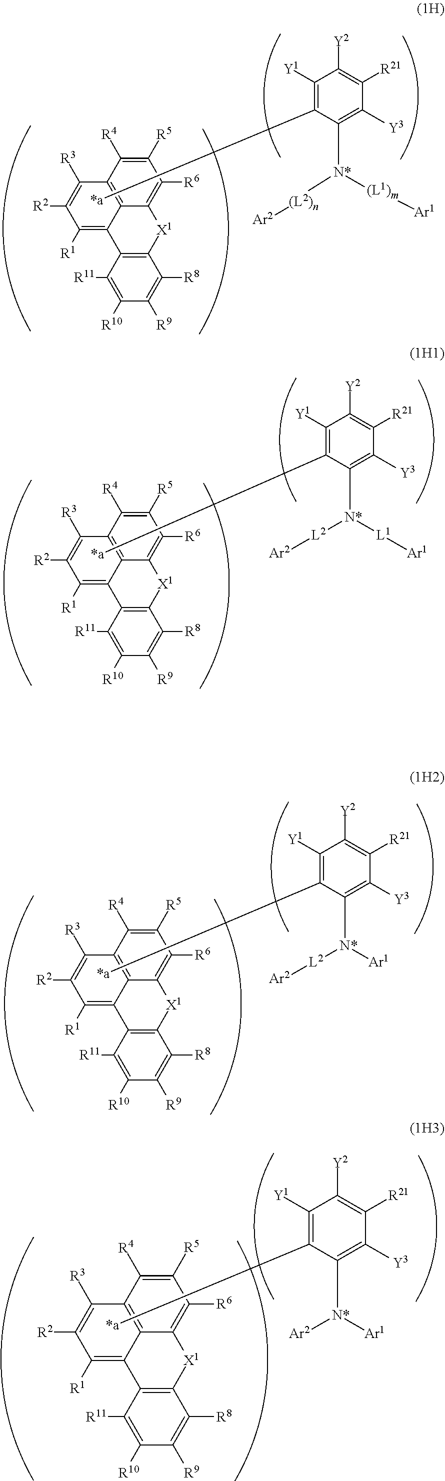

In formulae (1H), (1H-1), (1H-2), and (1H-3), X¹, R¹ to R⁶, R⁸ to R¹¹, R²¹, Y¹ to Y³, N*, L¹, L², Ar¹, Ar², *a, m, and n are as defined for the formula (1). Provided that one selected from R¹ to R⁶ is a single bond bonded to *a.

In addition, in one embodiment, compound (1) is represented by the following formula (1J1).

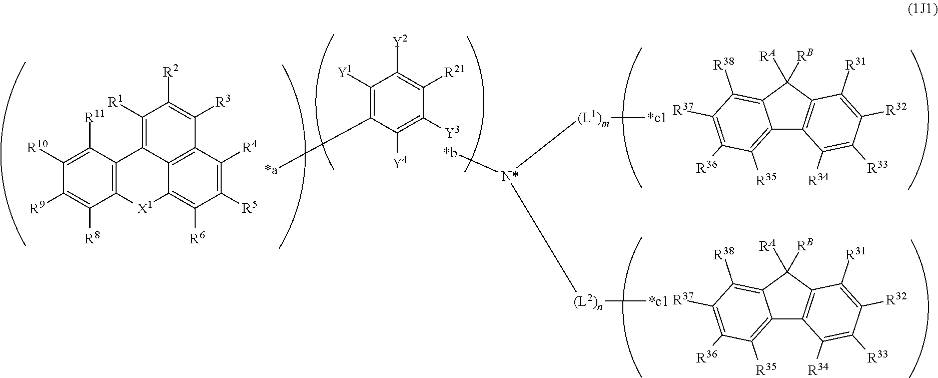

In formula (1J1), X¹, R¹ to R⁶, R⁸ to R¹¹, R²¹, Y¹ to Y⁴, N*, L¹, L², R³¹ to R³⁸, R^{A}, R^{B}, *a, *b, *c1, m, and n are as defined for the formula (1). Provided that R³¹ to R³⁸, R^{A}, R^{B}, and *c1 corresponding to Ar¹ and R³¹ to R³⁸, R^{A}, R^{B}, and *c1 corresponding to Ar² are the same as or different from each other.

In addition, in one embodiment, compound (1) is represented by the following formula (1J2).

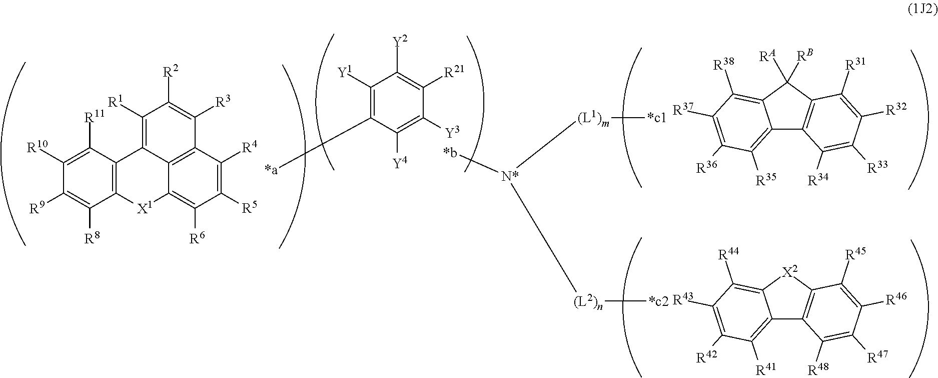

In formula (1J2), X¹, R¹ to R⁶, R⁸ to R¹¹, R²¹, Y¹ to Y⁴, N*, L¹, L², R³¹ to R³⁸, R^{A}, R^{B}X², R⁴¹ to R⁴⁸, *a, *b, *c1, *c2, m, and n are as defined for the formula (1).

In addition, in one embodiment, compound (1) is represented by the following formula (1J3).

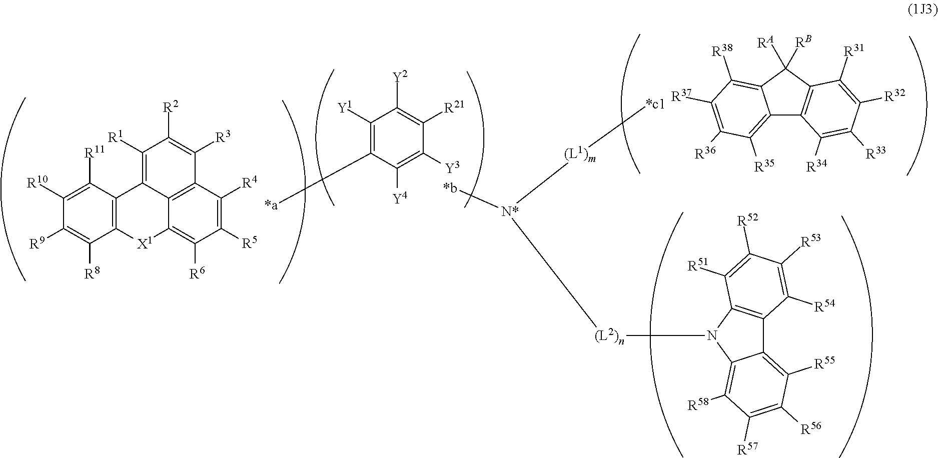

In formula (1J3), X¹, R¹ to R⁶, R⁸ to R¹¹, R²¹, Y¹ to Y⁴, N*, L¹, L², R³ to R³⁸, R^{A}, R^{B}, R⁵¹ to R⁵⁸, *a, *b, *c1, m, and n are as defined for the formula (1).

In addition, in one embodiment, compound (1) is represented by the following formula (1J4).

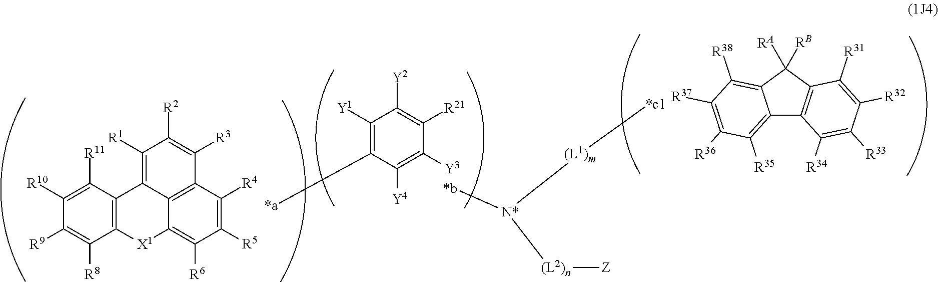

In formula (1J4), X¹, R¹ to R⁶, R⁸ to R¹¹, R²¹, Y¹ to Y⁴, N*, L¹, L², R³¹ to R³⁸, R^{A}, R^{B}, R⁵¹ to R⁵⁸, Z, *a, *b, *c1, m, and n are as defined for the formula (1).

In addition, in one embodiment, compound (1) is represented by the following formula (1J5).

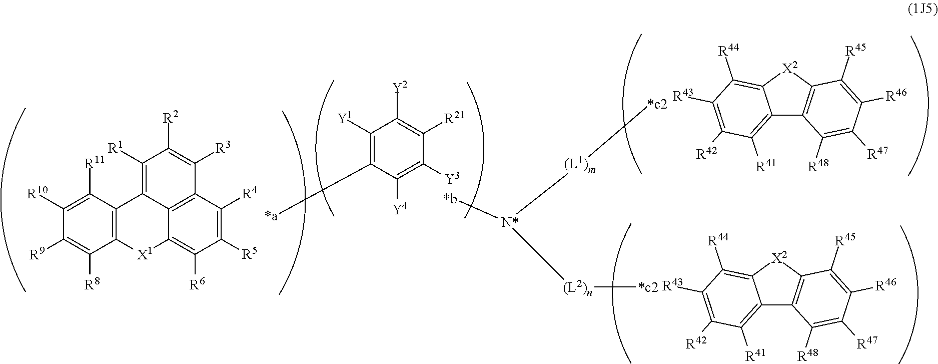

In formula (1J5), X¹, R¹ to R⁶, R⁸ to R¹¹, R²¹, Y¹ to Y⁴, N*, L¹, L², X², R⁴¹ to R⁴⁸, *a *b, *c2, m, and n are as defined for the formula (1). Provided that X², R⁴¹ to R⁴⁸, and *c2 corresponding to Ar¹ and X², R⁴¹ to R⁴⁸, and *c2 corresponding to Ar² are the same as or different from each other.

In addition, in one embodiment, compound (1) is represented by the following formula (1J6).

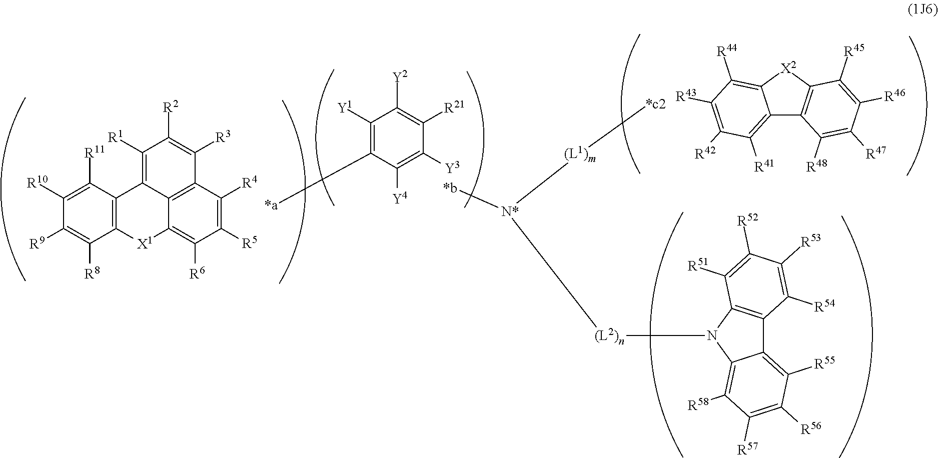

In formula (1J6), X¹, R¹ to R⁶, R⁸ to R¹¹, R²¹, Y¹ to Y⁴, N*, L¹, L², X², R⁴¹ to R⁴⁸, R⁵¹ to R⁵⁸, *a, *b, *c2, m, and n are as defined for the formula (1).

In addition, in one embodiment, compound (1) is represented by the following formula (1J7).

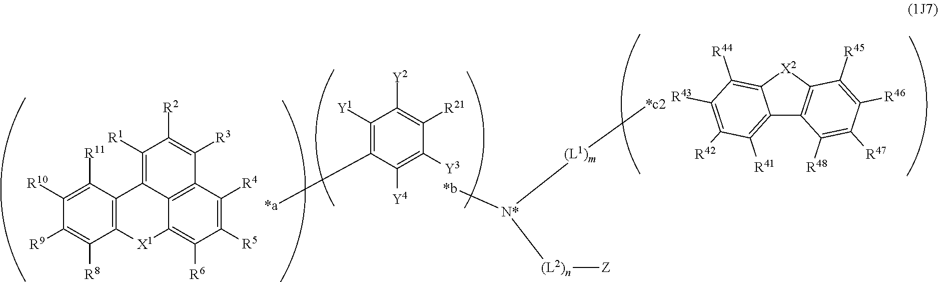

In formula (1J7), X¹, R¹ to R⁶, R⁸ to R¹¹, R²¹, Y¹ to Y⁴, N*, L¹, L², X², R⁴¹ to R⁴⁸, Z, *a, *b, *c2, m, and n are as defined for the formula (1).

In addition, in one embodiment, compound (1) is represented by the following formula (1J8).

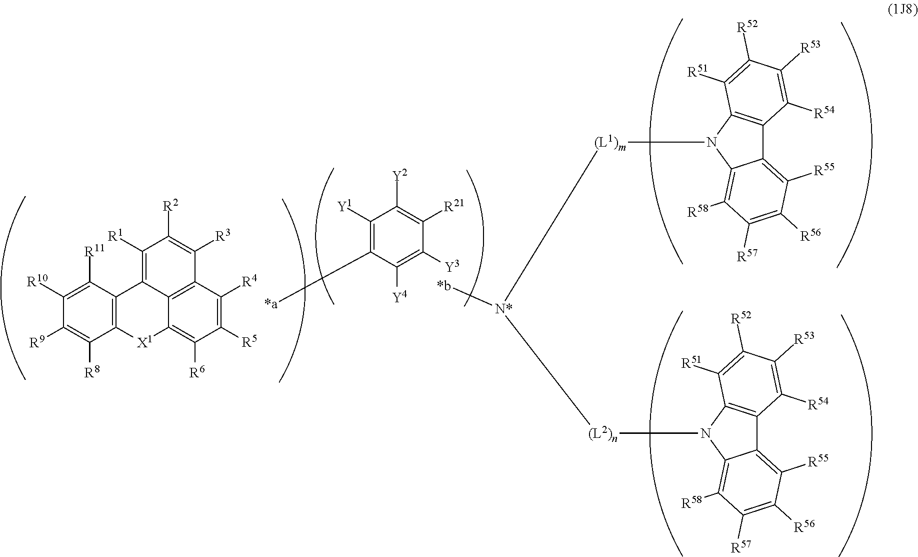

In formula (1J8), X¹, R¹ to R⁶, R⁸ to R¹¹, R²¹, Y¹ to Y⁴, N*, L¹, L², R⁵¹ to R⁵⁸, *a, *b, m, and n are as defined for the formula (1). Provided that R⁵¹ to R⁵⁸ corresponding to Ar¹ and R⁵¹ to R⁵⁸ corresponding to Ar² are the same as or different from each other.

In addition, in one embodiment, compound (1) is represented by the following formula (1J9).

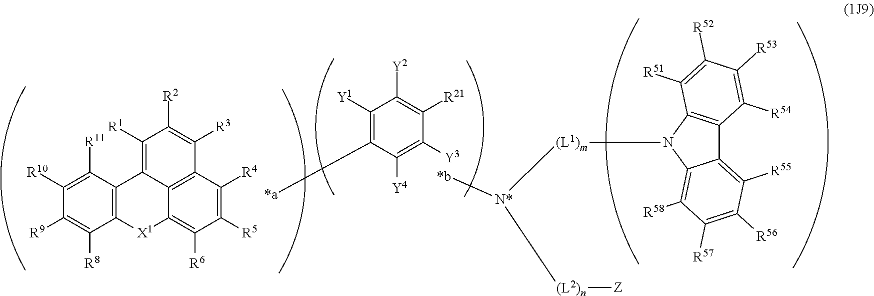

In formula (1J9), X¹, R¹ to R⁶, R⁸ to R¹¹, R²¹, Y¹ to Y⁴, N*, L¹, L², R¹¹ to R⁵⁸, Z, *a, *b, m, and n are as defined for the formula (1).

In addition, in one embodiment, compound (1) is represented by the following formula (1J10).

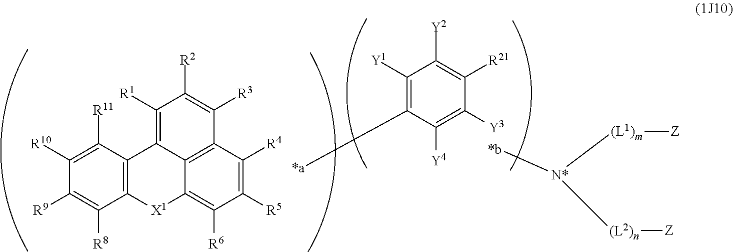

In formula (1J10), X¹, R¹ to R⁶, R⁸ to R¹¹, R²¹, Y¹ to Y⁴, N*, L¹, L², Z, *a, *b, m, and n are as defined for the formula (1). Provided that Z corresponding to Ar¹ and Z corresponding to Ar² are the same as or different from each other.

In the formula (1), Ar¹ and Ar² are preferably each independently represented by any one of the formula (1-a), (1-b), (1-c), or the following formulae (1-d1) to (1-d4).

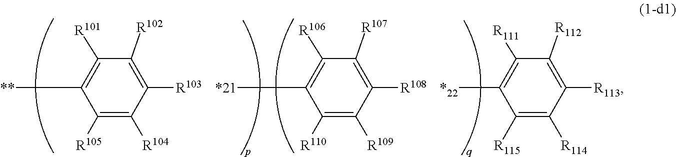

In formula (1-d1), ** represents a bonding site to one or both of L¹ and L².

In formula (1-d1), R¹⁰¹ to R¹⁰⁵, R¹⁰⁶ to R¹¹⁰, and R¹¹¹ to R¹¹⁵ are each independently a hydrogen atom, an unsubstituted alkyl group having 1 to 6 carbon atoms, or an unsubstituted aryl group having 6 to 12 ring carbon atoms, and are preferably hydrogen atoms. Provided that one selected from R¹⁰¹ to R¹⁰⁵ is a single bond bonded to *21, and one selected from R¹⁰⁶ to R¹¹⁰ is a single bond bonded to *22.

All of R¹⁰¹ to R¹⁰⁵ that are not the single bond, R¹⁰⁶ to R¹¹⁰ that are not the single bond, and R¹¹¹ to R¹¹⁵ that are not the single bond may be hydrogen atoms.

The details of the substituted or unsubstituted alkyl group having 1 to 6 carbon atoms are as described for R¹ to R⁶ and R⁸ to R¹⁰ of formula (1) except that the number of carbon atoms is 1 to 6.

The details of the substituted or unsubstituted aryl group having 1 to 12 ring carbon atoms are as described for R¹ to R⁶ and R⁸ to R¹⁰ of formula (1).

Each combination of adjacent moieties among R¹⁰¹ to R¹⁰⁵ that are not the single bond forms no ring, with the adjacent moieties not bonded to each other.

Each combination of adjacent moieties among R¹⁰⁶ to R¹¹⁰ that are not the single bond forms no ring, with the adjacent moieties not bonded to each other.

In formula (1-d1), p is 0 or 1, and q is 0 or 1.

When p is 0, and q is 1, *21 is a bonding site to one or both of L¹ and L².

When p is 1, and q is 0, one selected from R¹⁰¹ to R¹⁰⁵ is a single bond bonded to *22.

In partial structure C1, when Ar¹ is represented by formula (1-d1), it is preferable that L¹ be a substituted or unsubstituted naphthylene group, or m be 0.

In addition, in partial structure C2, when Ar² is represented by formula (1-d1), it is preferable that L² be a substituted or unsubstituted naphthylene group, or n be 0.

In partial structure C1 or C2, when p is 0, and q is 1, R¹⁰⁶ to R¹¹⁰ are preferably hydrogen atoms or unsubstituted alkyl groups having 1 to 6 carbon atoms.

In partial structure C1 or C2, when p is 1, and q is 0, R¹⁰¹ to R¹⁰⁵ that are not a single bond bonded to *22 are preferably hydrogen atoms or unsubstituted alkyl groups having 1 to 6 carbon atoms.

The group represented by formula (1-d1) is preferably represented by the following formulae. In the following formulae, R is omitted for simplicity.

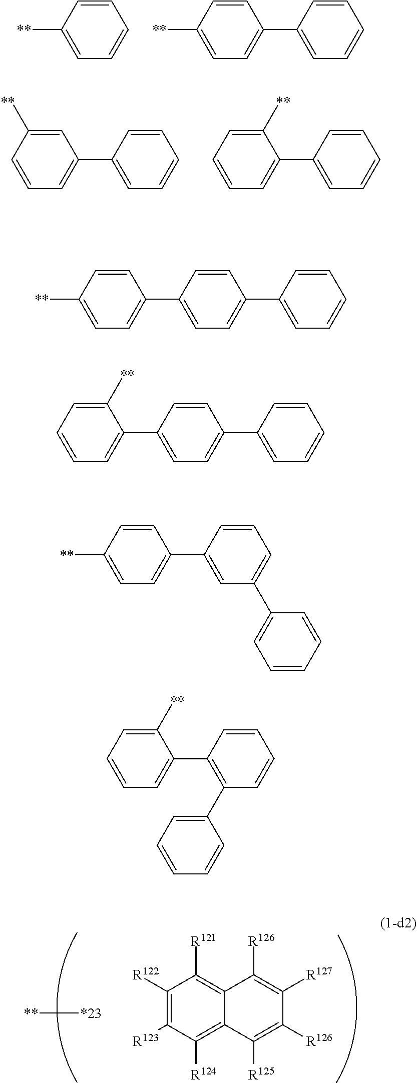

In formula (1-d2), ** represents a bonding site to one or both of L¹ and L².

In formula (1-d2), R¹²¹ to R¹²⁸ are each independently a hydrogen atom, a substituted or unsubstituted alkyl group having 1 to 6 carbon atoms, or a substituted or unsubstituted aryl group having 6 to 12 ring carbon atoms, and are preferably hydrogen atoms. Provided that one selected from R¹²¹ to R¹²⁸ is a single bond bonded to *23, and each combination of adjacent moieties among R¹²¹ to R¹²⁸ that are not the single bond forms no ring, with the adjacent moieties not bonded to each other.

All of R¹²¹ to R¹²⁸ that are not the single bond may be hydrogen atoms.

The details of the substituted or unsubstituted alkyl group having 1 to 6 carbon atoms are as described for R¹ to R⁶ and R⁸ to R¹⁰ of formula (1) except that the number of carbon atoms is 1 to 6.

The details of the substituted or unsubstituted aryl group having 1 to 12 ring carbon atoms are as described for R¹ to R⁶ and R⁸ to R¹⁰ of formula (1).

In partial structure C1, when Ar¹ is represented by formula (1-d2), it is preferable that L¹ be a substituted or unsubstituted phenylene group, L¹ be a substituted or unsubstituted biphenylene group, or m be 0.

In addition, in partial structure C2, when Ar² is represented by formula (1-d2), it is preferable that L² be a substituted or unsubstituted phenylene group, L² be a substituted or unsubstituted biphenylene group, or n be 0.

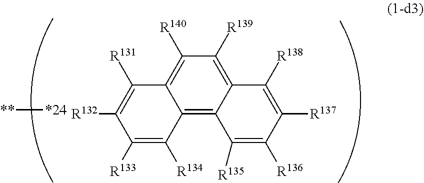

In formula (1-d3), ** represents a bonding site to one or both of L¹ and L².

In formula (1-d3), R¹³¹ to R¹⁴⁰ are each independently a hydrogen atom, a substituted or unsubstituted alkyl group having 1 to 6 carbon atoms, or a substituted or unsubstituted aryl group having 6 to 12 ring carbon atoms, and are preferably hydrogen atoms. Provided that one selected from R¹³¹ to R¹⁴⁰ is a single bond bonded to *24, and each combination of adjacent moieties among R¹³¹ to R¹⁴⁰ that are not the single bond forms no ring, with the adjacent moieties not bonded to each other.

All of R¹³¹ to R¹⁴⁰ that are not the single bond may be hydrogen atoms.

The details of the substituted or unsubstituted alkyl group having 1 to 6 carbon atoms are as described for R¹ to R⁶ and R⁸ to R¹⁰ of formula (1) except that the number of carbon atoms is 1 to 6.

The details of the substituted or unsubstituted aryl group having 1 to 12 ring carbon atoms are as described for R¹ to R⁶ and R⁸ to R¹⁰ of formula (1).

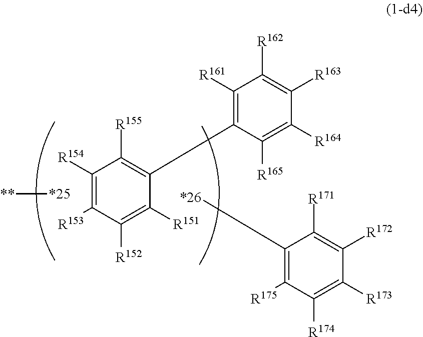

In formula (1-d4), ** represents a bonding site to one or both of L¹ and L².

In formula (1-d4), R¹⁵¹ to R¹⁵⁵ are each independently a hydrogen atom, an unsubstituted alkyl group having 1 to 6 carbon atoms, or an unsubstituted phenyl group, and are preferably hydrogen atoms. Provided that one selected from R¹⁵¹ to R¹⁵⁵ is a single bond bonded to *25, and another one selected from R¹⁵¹ to R¹⁵⁵ is a single bond bonded to *26.

Each combination of adjacent moieties among R¹⁵¹ to R¹⁵⁵ that are neither the single bond bonded to *25 nor the single bond bonded to *26 forms no ring, with the adjacent moieties not bonded to each other.

All of R¹⁵¹ to R¹⁵⁵ that are not the single bond may be hydrogen atoms.

The details of the substituted or unsubstituted alkyl group having 1 to 6 carbon atoms are as described for R¹ to R⁶ and R⁸ to R¹⁰ of formula (1) except that the number of carbon atoms is 1 to 6.

The details of the substituted or unsubstituted aryl group having 1 to 12 ring carbon atoms are as described for R¹ to R⁶ and R⁸ to R¹⁰ of formula (1).

In formula (1-d4), R¹⁶¹ to R¹⁶⁵ and R¹⁷¹ to R¹⁷⁵ are each independently a hydrogen atom or an unsubstituted alkyl group having 1 to 6 carbon atoms, and are preferably hydrogen atoms. Provided that at least one combination of adjacent two moieties among R¹⁶¹ to R¹⁶⁵ forms one or more unsubstituted benzene rings, with the adjacent moieties bonded to each other, or each combination of adjacent moieties among R¹⁶¹ to R¹⁶⁵ forms no ring, with the adjacent moieties not bonded to each other. At least one combination of adjacent two moieties among R¹⁷¹ to R¹⁷⁵ forms one or more unsubstituted benzene rings, with the adjacent moieties bonded to each other, or each combination of adjacent moieties among R¹⁷¹ to R¹⁷⁵ forms no ring, with the adjacent moieties not bonded to each other.

All of R¹⁶¹ to R¹⁶⁵ and R¹⁷¹ to R¹⁷⁵ may be hydrogen atoms.

The details of the substituted or unsubstituted alkyl group having 1 to 6 carbon atoms are as described for R¹ to R⁶ and R⁸ to R¹⁰ of formula (1) except that the number of carbon atoms is 1 to 6.

The details of the substituted or unsubstituted aryl group having 1 to 12 ring carbon atoms are as described for R¹ to R⁶ and R⁸ to R¹⁰ of formula (1).

In partial structure C1, when Ar¹ is represented by formula (1-d4), it is preferable that L¹ be a substituted or unsubstituted phenylene group, L¹ be a substituted or unsubstituted naphthylene group, or m be 0.

In addition, in partial structure C2, when Ar² is represented by formula (1-d4), it is preferable that L² be a substituted or unsubstituted phenylene group, L² be a substituted or unsubstituted naphthylene group, or n be 0.

Formula (1-d4) includes groups represented by the following formulae (1-d4a) to (1-d4e) and is preferably formula (1-d4a), (1-d4b), or (1-d4e).

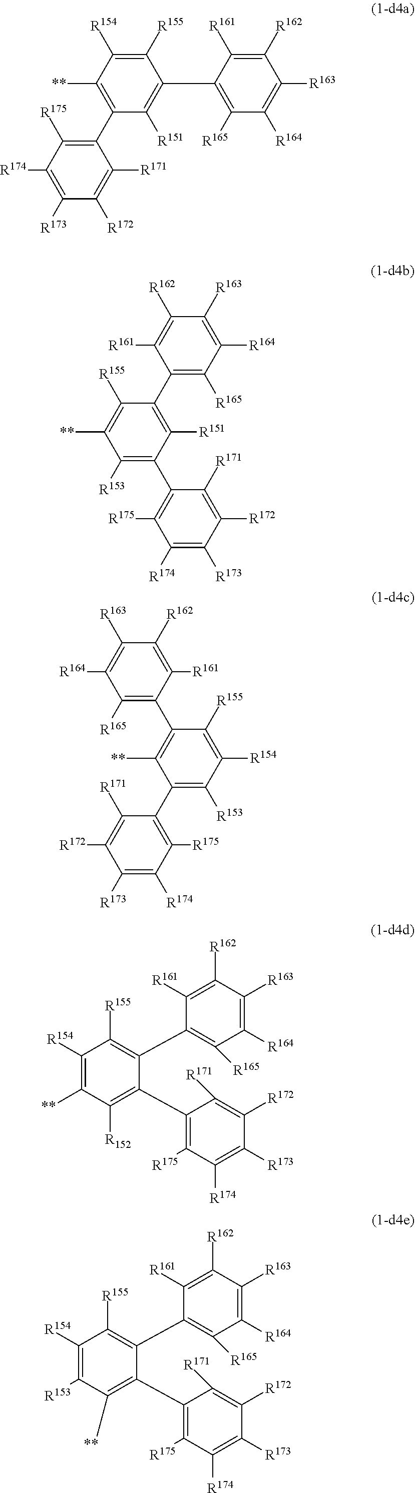

In one embodiment, among combinations of Ar¹ and Ar² in compound (1), the [d], [g], [i], and [j] are represented by any of the combinations [d1] to [d4], [g1] to [g4], [i1] to [i4], and [j1] to [j10] shown below.
[d1]: (1-a)/(1-d2)
[d2]: (1-a)/(1-d2)
[d3]: (1-a)/(1-d3)
[d4]: (1-a)/(1-d4)
[g1]: (1-b)/(1-d1)
[g2]: (1-b)/(1-d2)
[g3]: (1-b)/(1-d3)
[g4]: (1-b)/(1-d4)
[i1]: (1-c)/(1-d1)
[i2]: (1-c)/(1-d2)
[i3]: (1-c)/(1-d3)
[i4]: (1-c)/(1-d4)
[j1]: (1-d1)/(1-d1)
[j2]: (1-d1)/(1-d2)
[j3]: (1-d1/(1-d3)
[j4]: (1-d1)/(1-d4)
[j5]: (1-d2)/(1-d2)
[j6]: (1-d2)/(1-d3)
[j7]: (1-d2)/(1-d4)
[j8]: (1-d3)/(1-d3)
[j9]: (1-d3)/(1-d4)
[j10]: (1-d4)/(1-d4)

Among them, [d1], [d2], [d4], [g1], [g2], [g4], [i1], [i2], [i4], [j1], [j2], [j4], [j5], [j7], and [j10] are preferable.

In the formula (1), at least one of Ar¹ and Ar² is preferably represented by the formula (1-d1) and is represented by, for example, the following formula (1K1).

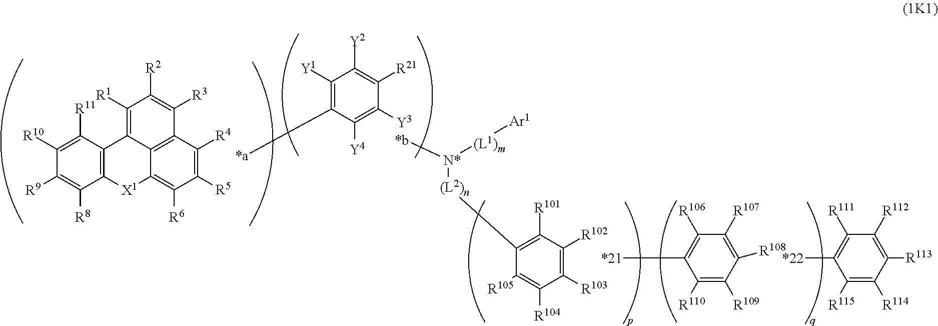

In formula (1K1), X¹, R¹ to R⁶, R⁸ to R¹¹, R²¹, Y¹ to Y⁴, N*, L¹, L², Ar¹, *a, *b, m, and n are as defined for the formula (1); and R¹⁰¹ to R¹⁰⁵, R¹⁰⁶ to R¹¹⁰, R¹¹¹ to R¹¹⁵, *21, *22, p, and q are as defined for the formula (1-d1).

In the formula (1), Ar¹ and Ar² are preferably represented by the formula (1-d1) and is represented by, for example, the following formula (1K2).

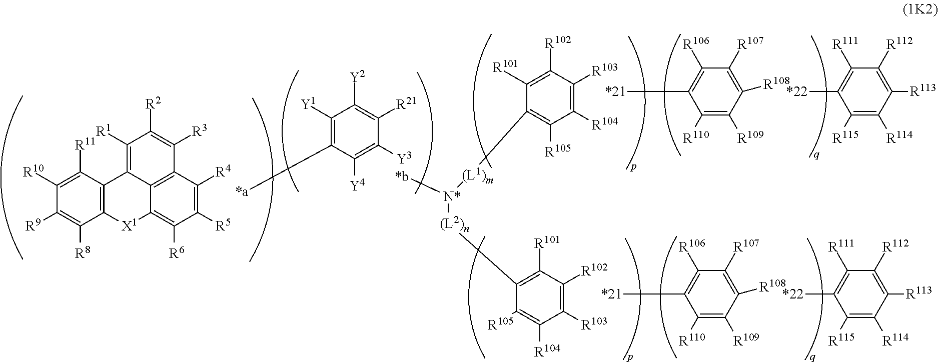

In formula (1K2), X¹, R¹ to R⁶, R⁸ to R¹¹, R²¹, Y¹ to Y⁴, N*, L¹, L², *a, *b, m, and n are as defined for the formula (1); and R¹⁰¹ to R¹⁰⁵, R¹⁰⁶ to R¹¹⁰, R¹¹¹ to R¹¹⁵, *21, *22, p, and q are as defined for the formula (1-d1). Provided that the partial structure bonded to “-(L¹)ₘ-” and the partial structure bonded to “-(L²)ₙ-” are the same as or different from each other.

In one embodiment, partial structure A/partial structure B/partial structures C1 and C2 in the compound (1) are the following combination.

Partial structure A: any of the formulae (1x-1) to (1x-10)

Partial structure B: a m-phenylene group or an o-phenylene group

Partial structures C1 and C2: a combination of any of the [k1] to [k10] and any of the [a] to [j], [d1] to [d4], [g1] to [g4], [i1] to [i4], and [j1] to [j4]

Preferably, partial structure A/partial structure B/partial structures C1 and C2 in the compound (1) are the following combination.

Partial structure A: any of the formulae (1x-2) to (1x-4) and (1x-10)

Partial structure B: a m-phenylene group

Partial structures C1 and C2: a combination of any of the [k1], [k2], [k4], [k5], [k7], and [k10] and any of the [a] to [g], [i], [j], [d1], [d2], [d4], [g1], [g2], [g4], [i1], [i2], [i4], [j1], [j2], [j4], [j5], [j7], and [j10]

In one embodiment, R¹ to R⁶ and R⁸ to R¹¹ that are not the single bond, Y¹ to Y⁴ that are not the single bond, R³¹ to R³⁸ that are not the single bond, R⁴¹ to R⁴⁸ that are not the single bond, R⁵¹ to R⁵⁸ that are not the single bond, R¹⁰¹ to R¹⁰⁵ that are not the single bond, R¹⁰⁶ to R¹¹⁰, R¹¹¹ to R¹¹⁵ that are not the single bond, R¹²¹ to R¹²⁸ that are not the single bond, R¹³¹ to R¹⁴⁰ that are not the single bond, R¹⁵¹ to R¹⁵⁵, R¹⁶¹ to R¹⁶⁵, and R¹⁷¹ to R¹⁷⁵ that are not the single bond are all hydrogen atoms.

In one embodiment, compound (1) includes at least one deuterium atom.

The deuterium atom included in inventive compound (1) will be described in detail later.

In one embodiment of inventive compound (1), at least one of the following (1) to (9) is a deuterium atom.
(1) Hydrogen atoms represented by R¹ to R⁶, R⁸ to R¹¹, R²¹, R³ to R³⁸, R⁴¹ to R⁴⁸, R⁵¹ to R⁵⁸, R¹⁰¹ to R¹⁰⁵, R¹⁰⁶ to R¹¹⁰, R¹¹¹ to R¹¹⁵, R¹²¹ to R¹²⁸, R¹³¹ to R¹⁴⁰, R¹⁵¹ to R¹⁵⁵, R¹⁶¹ to R¹⁶⁵, R¹⁷¹ to R¹⁷⁵, R^{A1} to R^{A3}, R^{A4} to R^{A8}, R^{B1} to R^{B3}, R^{B4} to R^{B8}, R^{C1} to R^{C4}, Y¹ to Y⁴, and Z;
(2) hydrogen atoms directly bonded to alkyl groups represented by R¹ to R⁶, R⁸ to R¹¹, R³¹ to R³⁸, R⁴¹ to R⁴⁸, R⁵¹ to R⁵⁸, R¹⁰¹ to R¹⁰⁵, R¹⁰⁶ to R¹¹⁰, R¹¹¹ to R¹¹⁵, R¹²¹ to R¹²⁸, R¹³¹ to R¹⁴⁰, R¹⁵¹ to R¹⁵⁵, R¹⁶¹ to R¹⁶⁵, R¹⁷¹ to R¹⁷⁵, R^{A}, and R^{B};
(3) hydrogen atoms directly bonded to aryl groups represented by R¹ to R⁶, R⁸ to R¹¹, R³¹ to R³⁸, R⁴¹ to R⁴⁸, R⁵¹ to R⁵⁸, R¹⁰¹ to R¹⁰⁵, R¹⁰⁶ to R¹¹⁰, R¹¹¹ to R¹¹⁵, R¹²¹ to R¹²⁸, R¹³¹ to R¹⁴⁰, R¹⁵¹ to R¹⁵⁵, R¹⁶¹ to R¹⁶⁵, R¹⁷¹ to R¹⁷⁵, R^{A}, R^{B}, and Z;
(4) hydrogen atoms directly bonded to heterocyclic groups represented by R^{A} and R^{B};
(5) hydrogen atoms directly bonded to substituents of alkyl groups represented by R¹ to R⁶, R⁸ to R¹¹, R³¹ to R³⁸, R⁴¹ to R⁴⁸, R⁵¹ to R⁵⁸, R¹⁰¹ to R¹⁰⁵, R¹⁰⁶ to R¹¹⁰, R¹¹¹ to R¹¹⁵, R¹²¹ to R¹²⁸, R¹³¹ to R¹⁴⁰, R¹⁵¹ to R¹⁵⁵, R¹⁶¹ to R¹⁶⁵, R¹⁷¹ to R¹⁷⁵, R^{A}, and R^{B};
(6) hydrogen atoms directly bonded to substituents of aryl groups represented by R¹ to R⁶, R⁸ to R¹¹, R³¹ to R³⁸, R⁴¹ to R⁴⁸, R⁵¹ to R⁵⁸, R¹⁰¹ to R¹⁰⁵, R¹⁰⁶ to R¹¹⁰, R¹¹¹ to R¹¹⁵, R¹²¹ to R¹²⁸, R¹³¹ to R¹⁴⁰, R¹⁵¹ to R¹⁵⁵, R¹⁶¹ to R¹⁶⁵, R¹⁷¹ to R¹⁷⁵, R^{A}, and R^{B};
(7) hydrogen atoms directly bonded to substituents of heterocyclic groups represented by R^{A} and R^{B};
(8) hydrogen atoms directly bonded to phenylene groups, naphthylene groups, and biphenylene groups represented by L¹ and L⁴; and
(9) hydrogen atoms directly bonded to substituents of phenylene groups, naphthylene groups, and biphenylene groups represented by L¹ and L⁴.

As described above, the term “hydrogen atom” used herein includes a protium atom, a deuterium atom, and a tritium atom. The inventive compound may include a naturally occurring deuterium atom.

A deuterium atom may be intentionally introduced into the inventive compound by using a deuterated compound for some or all of raw material compounds.

The deuteration rate of the inventive compound depends on the deuteration rate of a raw material compound used. Even when a raw material with a predetermined deuteration rate is used, a naturally occurring protium isotope may be contained at a certain proportion. Accordingly, embodiments of the deuteration rate of the inventive compound shown below include ratios taking into account a trace amount of naturally occurring isotopes besides ratios determined by simply counting the number of deuterium atoms represented by a chemical formula.

The deuteration rate of the inventive compound is preferably 1% or more, more preferably 3% or more, still more preferably 5% or more, even more preferably 10% or more, and further preferably 50% or more.

The inventive compound may be a deuteride in which all hydrogen atoms are deuterium atoms (that is, the deuteration rate of inventive compound is 100%).

The inventive compound may be a mixture including deuterated compound and a non-deuterated compound or a mixture of two or more compounds having different deuteration rates. The deuteration rate of such a mixture is preferably 1% or more, more preferably 3% or more, still more preferably 5% or more, even more preferably 10% or more, and further preferably 50% or more, and less than 100%.

Each ratio of the number of deuterium atoms to the total number of hydrogen atoms in the inventive compound is preferably 1% or more, more preferably 3% or more, still more preferably 5% or more, and even more preferably 10% or more, and 100% or less.

Details of the substituent (arbitrary substituent) in the case of using the phrase “substituted or unsubstituted” included in the definitions of the above formulae are as described in the “substituent for the case of “substituted or unsubstituted”.”

The inventive compound can be easily produced by those skilled in the art with reference to the synthesis examples described below and known synthesis methods.

Specific examples of the inventive compound are shown below, but the inventive compound is not limited to the following exemplary compounds.

In the following specific examples, D represents a deuterium atom.

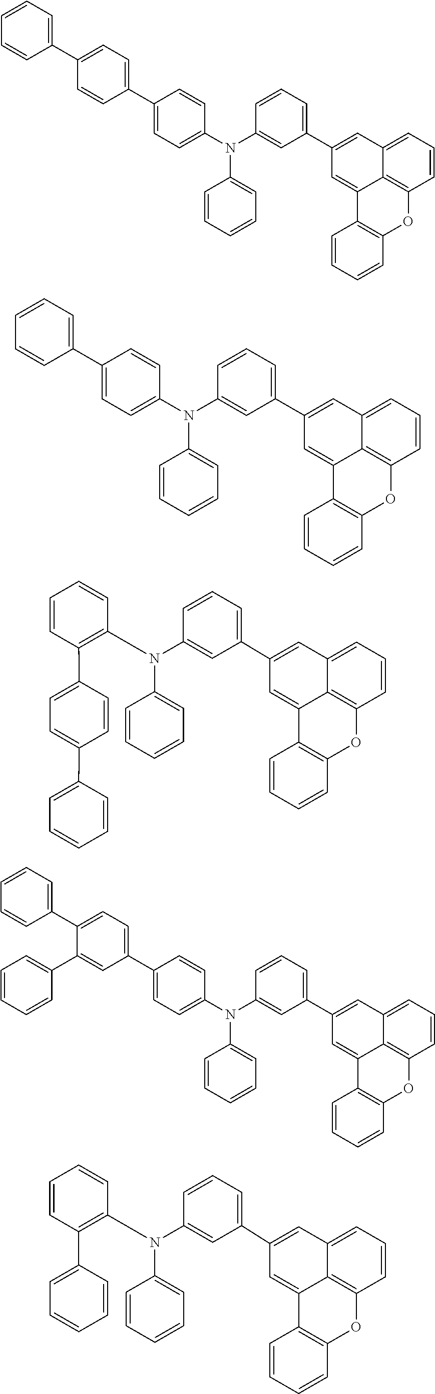

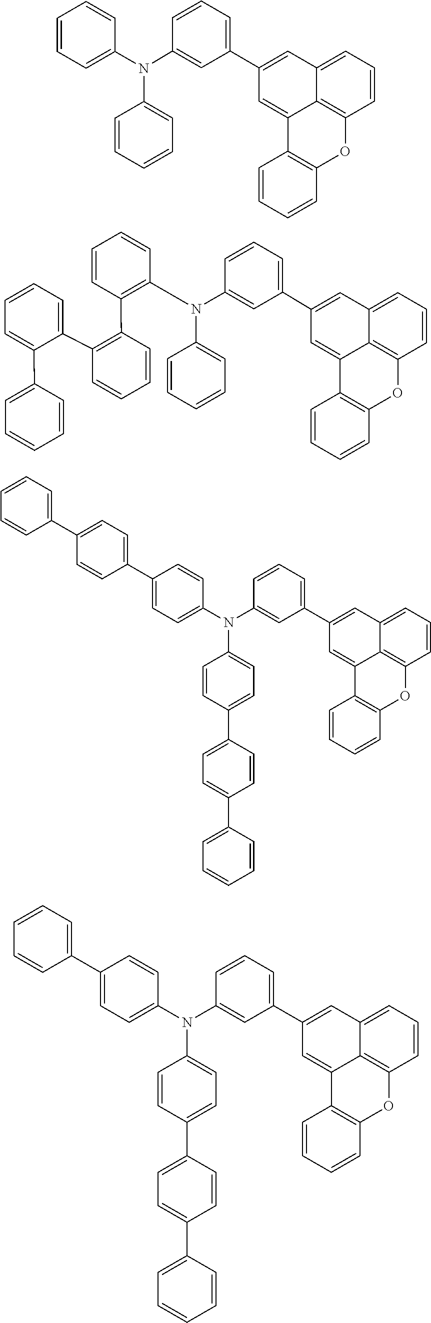

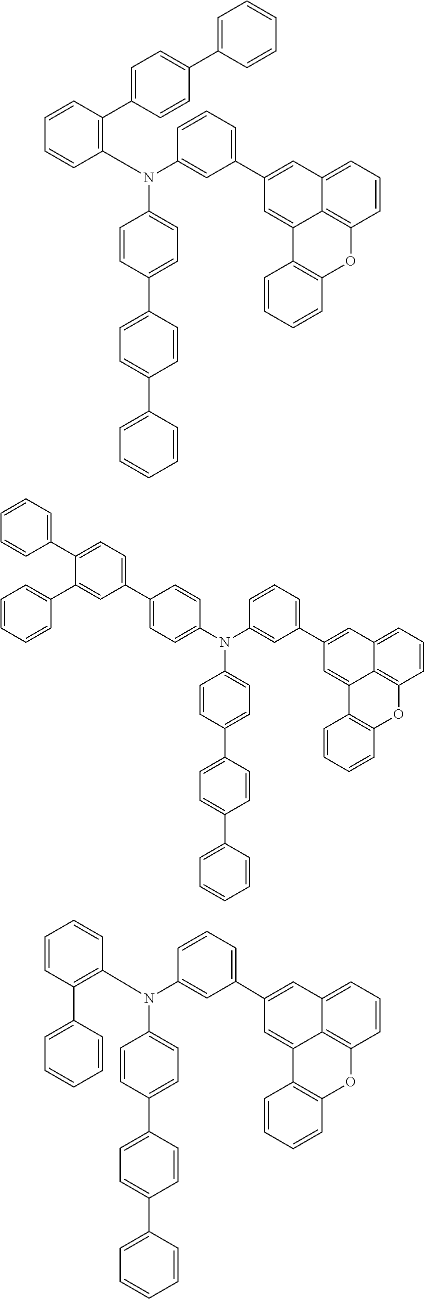

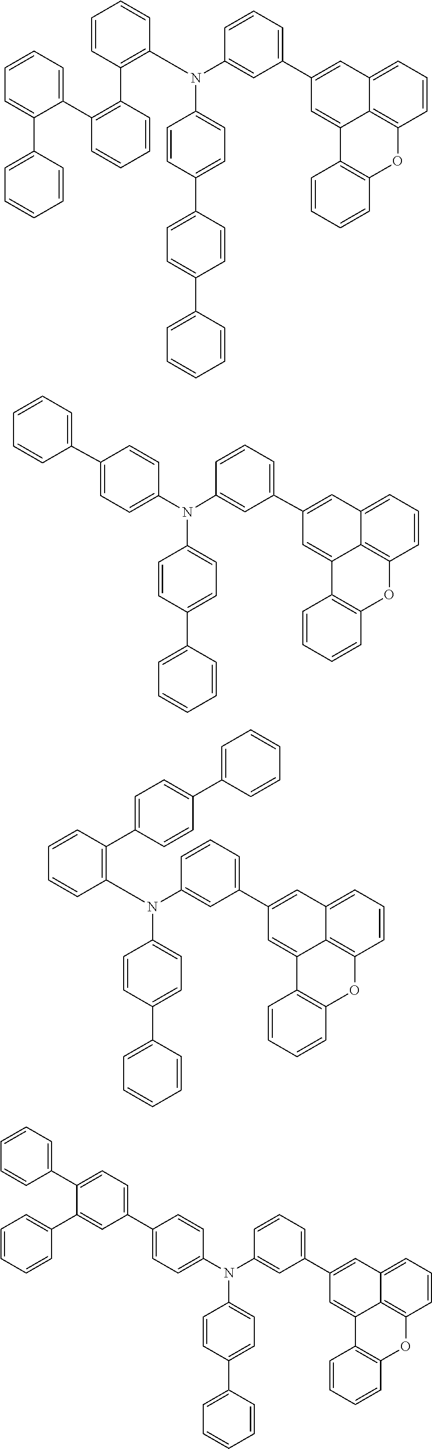 [SMILES: c1ccc(-c2ccc(-c3ccc(N(c4cccc(-c5cc6c7c(cccc7c5)Oc5ccccc5-6)c4)c4ccccc4-c4ccccc4-c4ccccc4-c4ccccc4)cc3)cc2)cc1 ; c1ccc(-c2ccc(N(c3ccc(-c4ccccc4)cc3)c3cccc(-c4cc5c6c(cccc6c4)Oc4ccccc4-5)c3)cc2)cc1 ; c1ccc(-c2ccc(-c3ccccc3N(c3ccc(-c4ccccc4)cc3)c3cccc(-c4cc5c6c(cccc6c4)Oc4ccccc4-5)c3)cc2)cc1 ; c1ccc(-c2ccc(N(c3ccc(-c4ccc(-c5ccccc5)c(-c5ccccc5)c4)cc3)c3cccc(-c4cc5c6c(cccc6c4)Oc4ccccc4-5)c3)cc2)cc1]

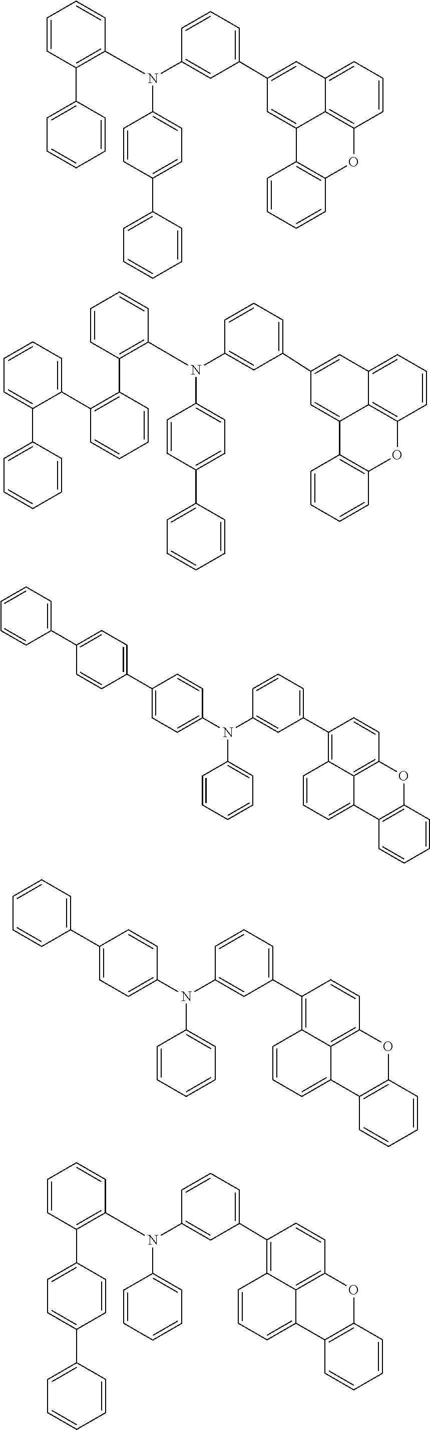

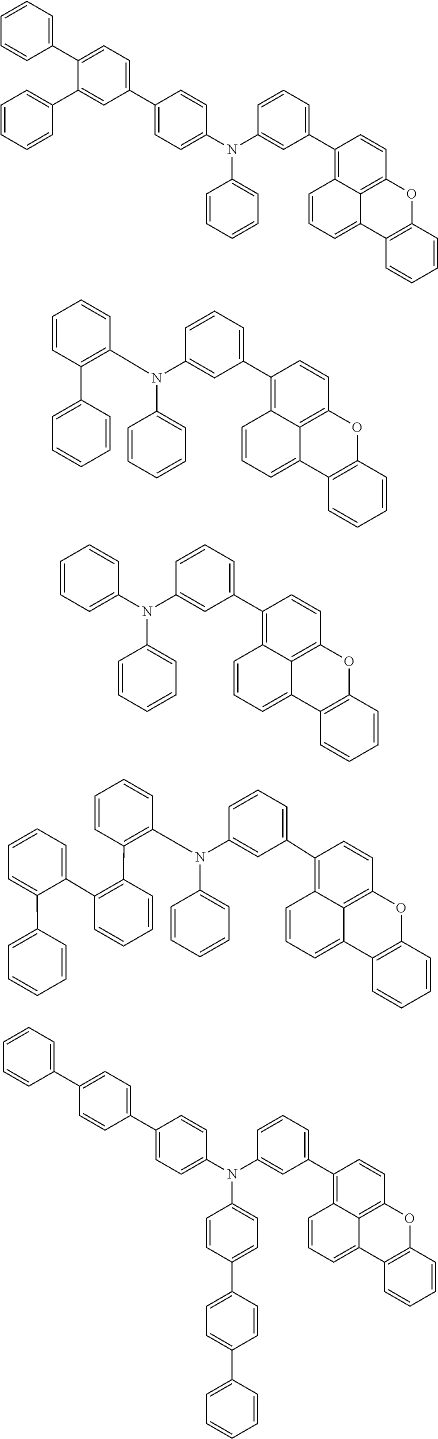

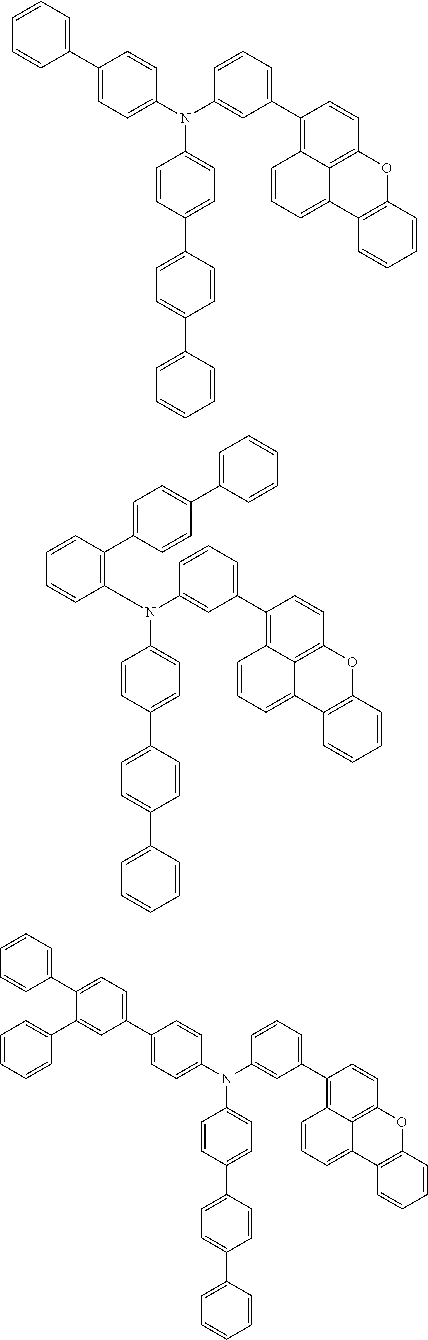 [SMILES: c1ccc(-c2ccc(-c3ccc(N(c4ccc(-c5ccccc5)cc4)c4cccc(-c5ccc6c7c(cccc57)-c5ccccc5O6)c4)cc3)cc2)cc1 ; c1ccc(-c2ccc(-c3ccc(N(c4cccc(-c5ccc6c7c(cccc57)-c5ccccc5O6)c4)c4ccccc4-c4ccc(-c5ccccc5)cc4)cc3)cc2)cc1 ; c1ccc(-c2ccc(-c3ccc(N(c4ccc(-c5ccc(-c6ccccc6)c(-c6ccccc6)c5)cc4)c4cccc(-c5ccc6c7c(cccc57)-c5ccccc5O6)c4)cc3)cc2)cc1]

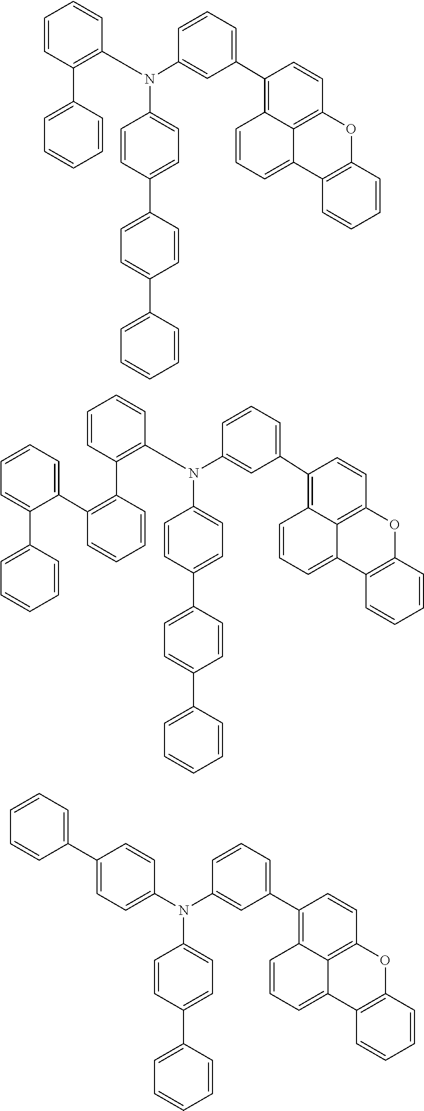

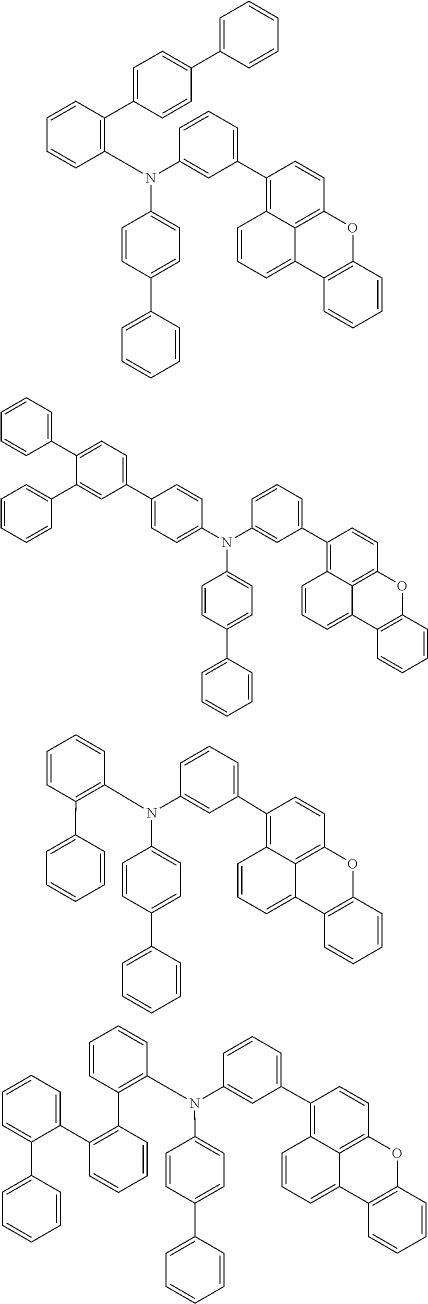

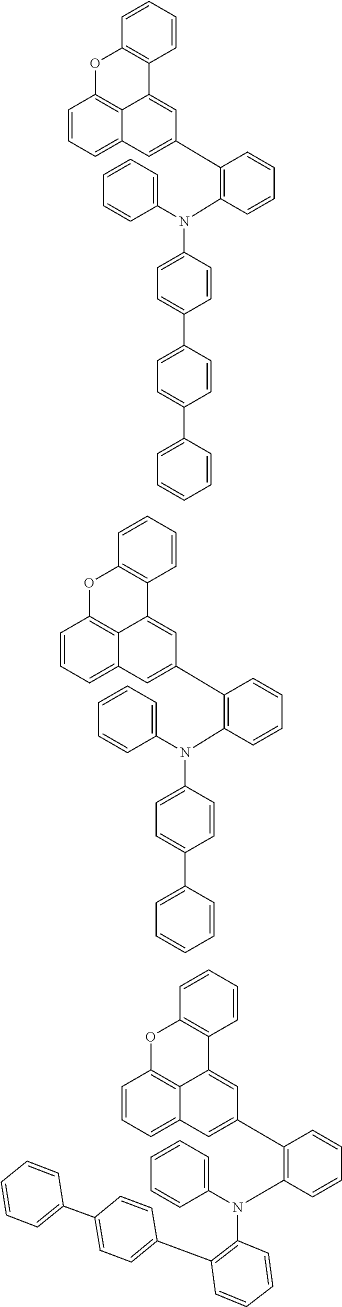

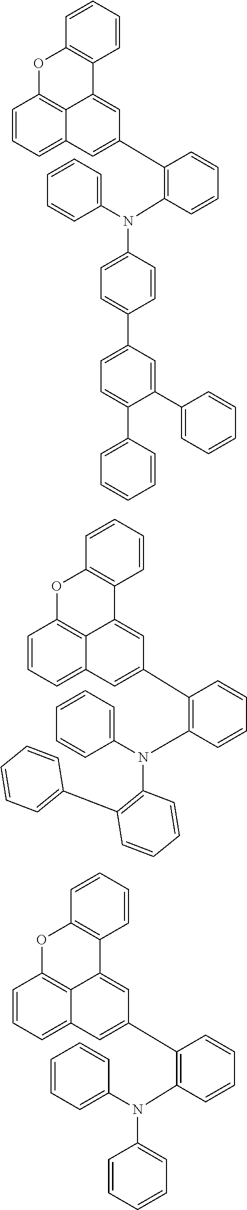

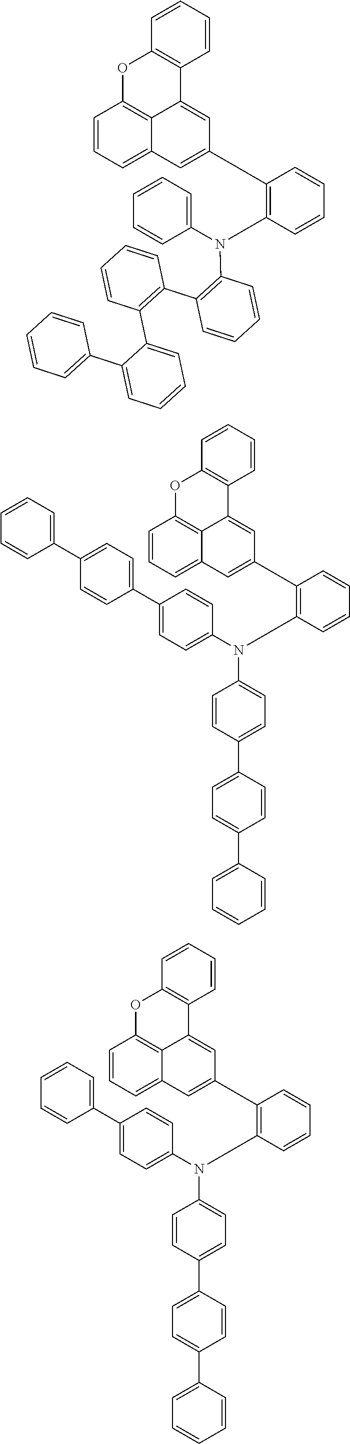

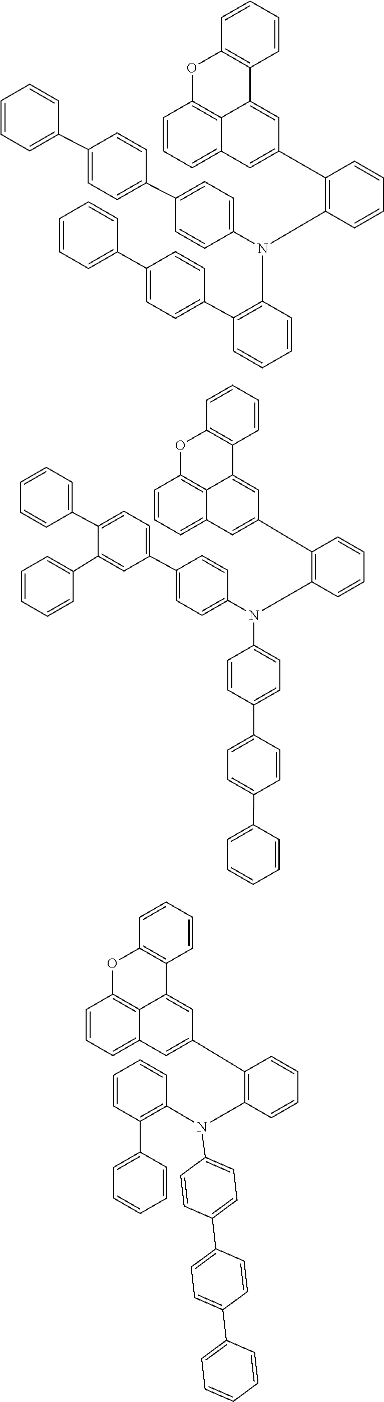

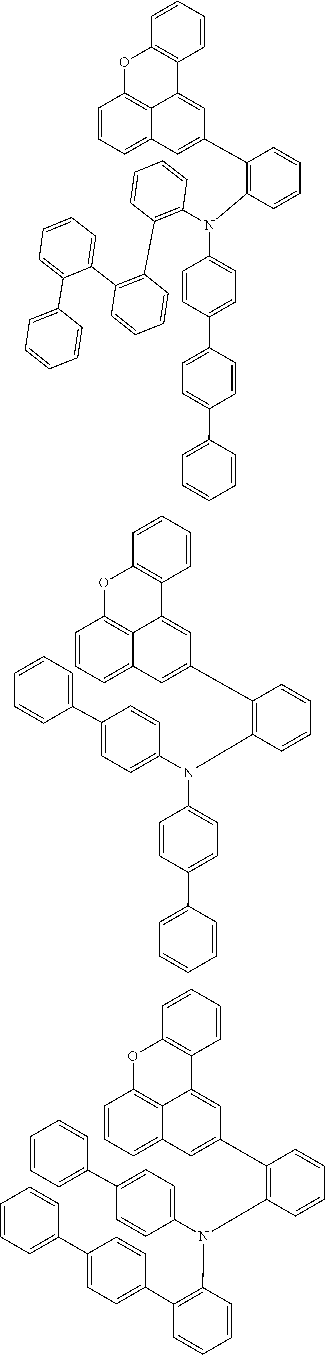

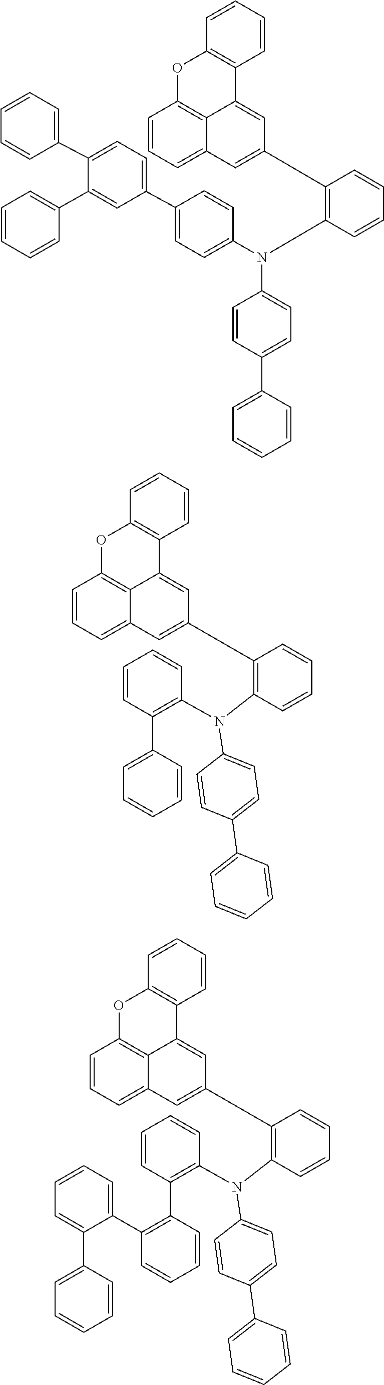 [SMILES: c1ccc(-c2ccc(N(c3ccc(-c4ccc(-c5ccccc5)c(-c5ccccc5)c4)cc3)c3ccccc3-c3cc4c5c(cccc5c3)Oc3ccccc3-4)cc2)cc1 ; c1ccc(-c2ccc(N(c3ccccc3-c3ccccc3)c3ccccc3-c3cc4c5c(cccc5c3)Oc3ccccc3-4)cc2)cc1 ; c1ccc(-c2ccc(N(c3ccccc3-c3cc4c5c(cccc5c3)Oc3ccccc3-4)c3ccccc3-c3ccccc3-c3ccccc3-c3ccccc3)cc2)cc1]

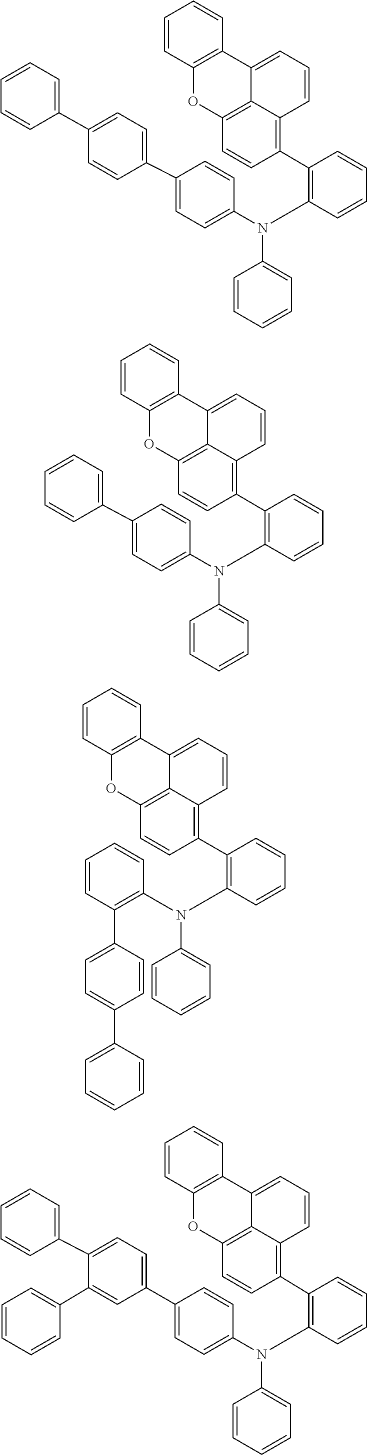 [SMILES: c1ccc(-c2ccc(-c3ccc(N(c4ccccc4)c4ccccc4-c4ccc5c6c(cccc46)-c4ccccc4O5)cc3)cc2)cc1 ; c1ccc(-c2ccc(N(c3ccccc3)c3ccccc3-c3ccc4c5c(cccc35)-c3ccccc3O4)cc2)cc1 ; c1ccc(-c2ccc(-c3ccccc3N(c3ccccc3)c3ccccc3-c3ccc4c5c(cccc35)-c3ccccc3O4)cc2)cc1 ; c1ccc(-c2ccc(-c3ccc(N(c4ccccc4)c4ccccc4-c4ccc5c6c(cccc46)-c4ccccc4O5)cc3)cc2-c2ccccc2)cc1]

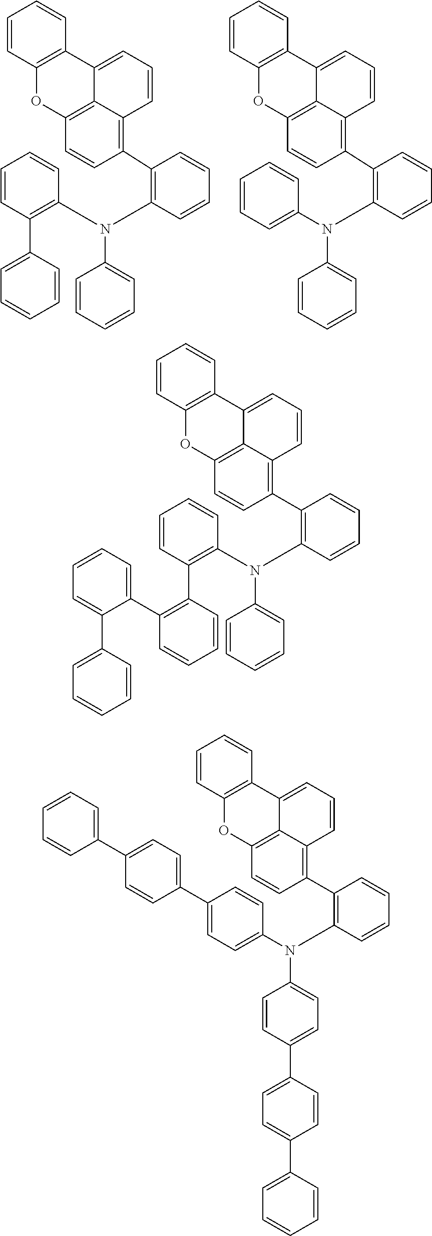

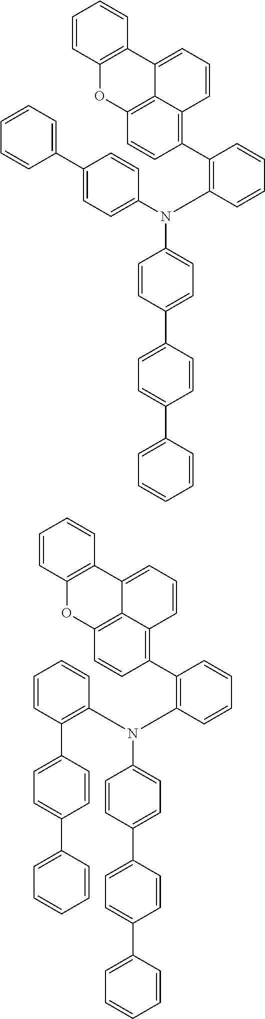 [SMILES: c1ccc(-c2ccc(-c3ccc(N(c4ccc(-c5ccccc5)cc4)c4ccccc4-c4ccc5c6c(cccc46)-c4ccccc4O5)cc3)cc2)cc1 ; c1ccc(-c2ccc(-c3ccc(N(c4ccccc4-c4ccc(-c5ccccc5)cc4)c4ccccc4-c4ccc5c6c(cccc46)-c4ccccc4O5)cc3)cc2)cc1]

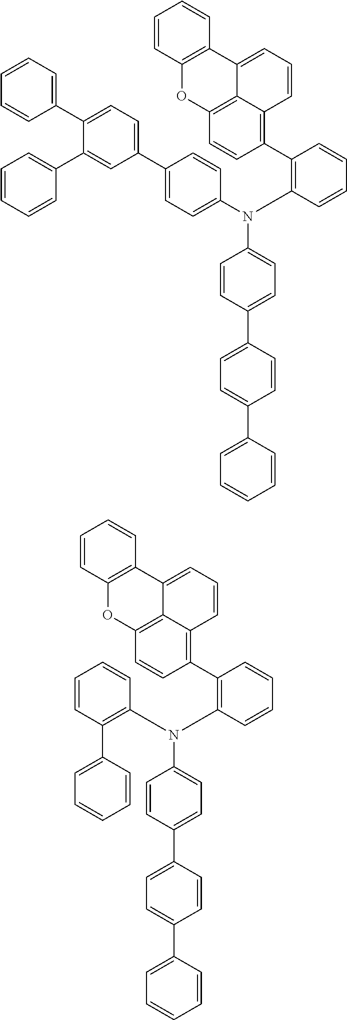 [SMILES: c1ccc(-c2ccc(-c3ccc(N(c4ccc(-c5ccc(-c6ccccc6)c(-c6ccccc6)c5)cc4)c4ccccc4-c4ccc5c6c(cccc46)-c4ccccc4O5)cc3)cc2)cc1 ; c1ccc(-c2ccc(-c3ccc(N(c4ccccc4-c4ccccc4)c4ccccc4-c4ccc5c6c(cccc46)-c4ccccc4O5)cc3)cc2)cc1]

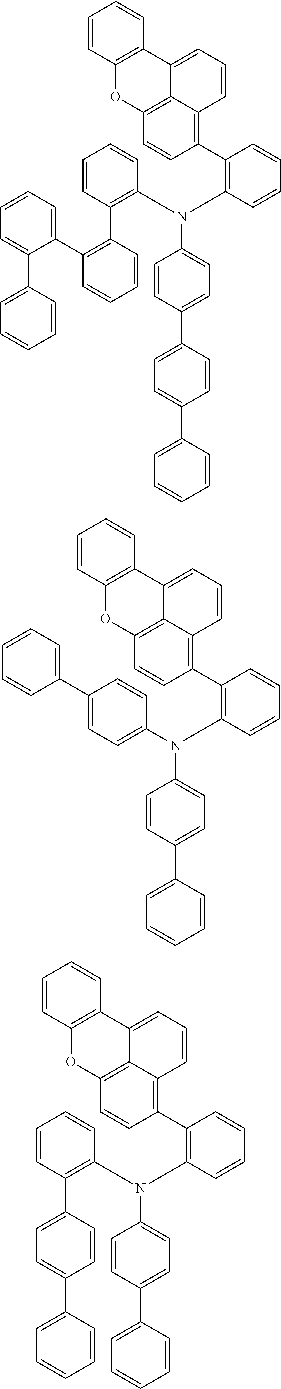 [SMILES: c1ccc(-c2ccc(-c3ccc(N(c4ccccc4-c4ccccc4-c4ccccc4-c4ccccc4)c4ccccc4-c4ccc5c6c(cccc46)-c4ccccc4O5)cc3)cc2)cc1 ; c1ccc(-c2ccc(N(c3ccc(-c4ccccc4)cc3)c3ccccc3-c3ccc4c5c(cccc35)-c3ccccc3O4)cc2)cc1 ; c1ccc(-c2ccc(-c3ccccc3N(c3ccc(-c4ccccc4)cc3)c3ccccc3-c3ccc4c5c(cccc35)-c3ccccc3O4)cc2)cc1]

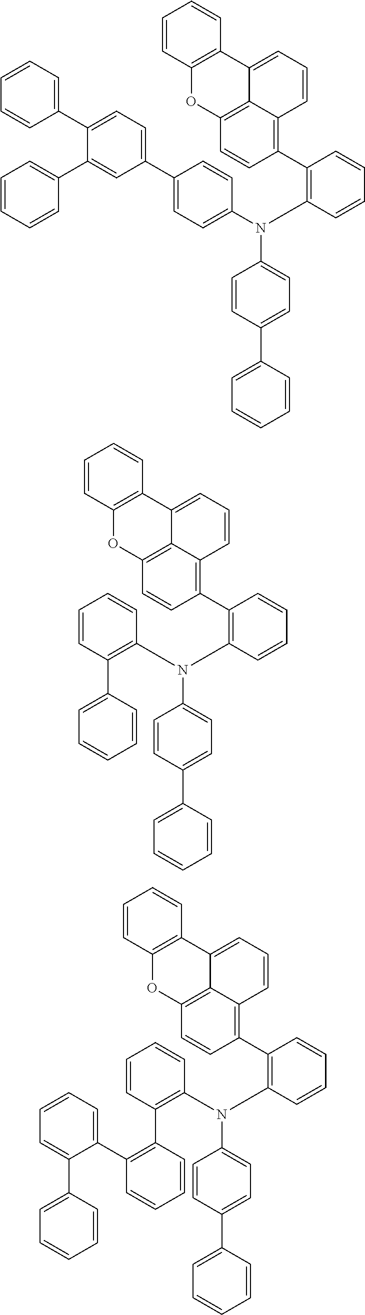

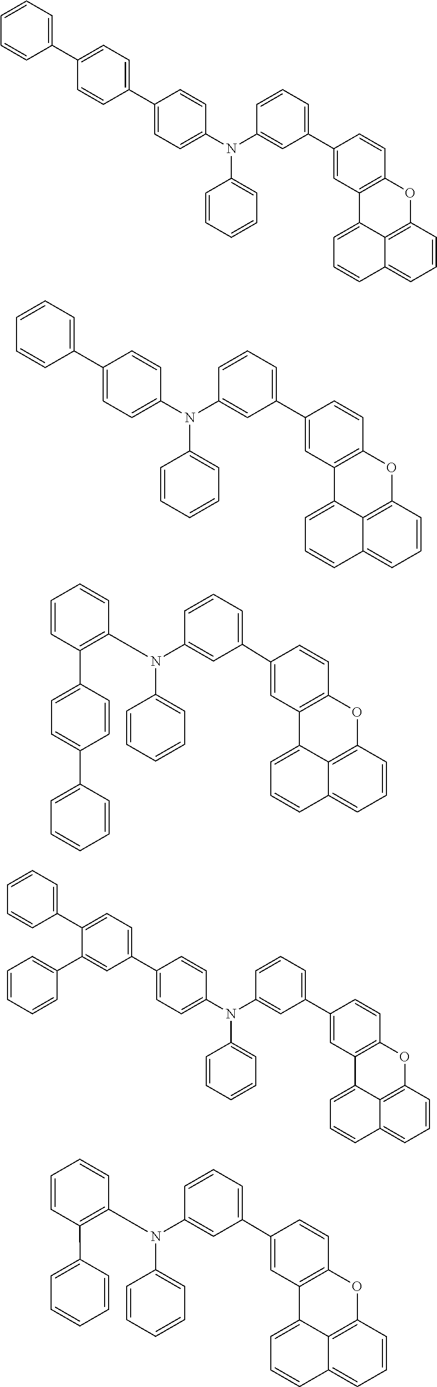

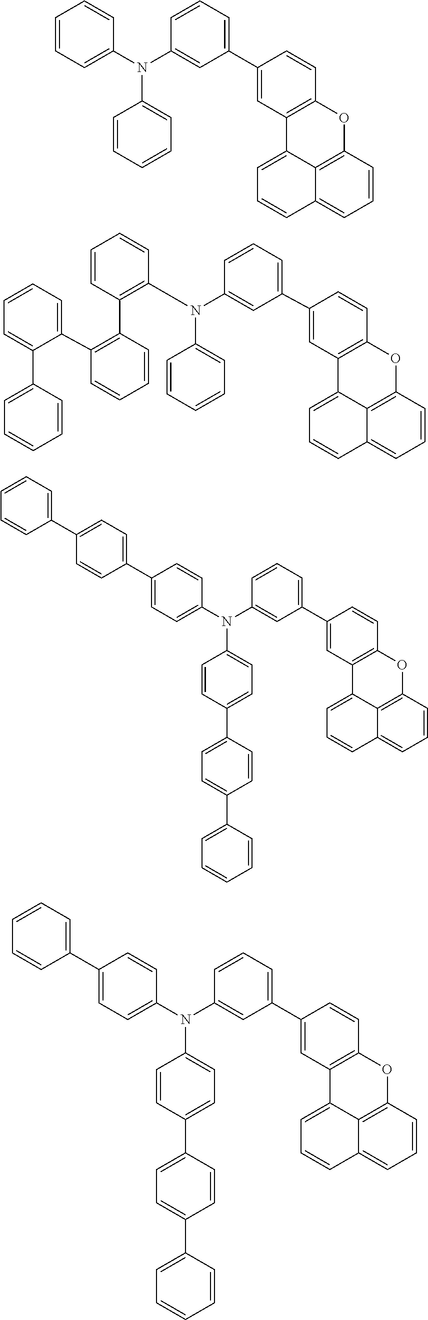

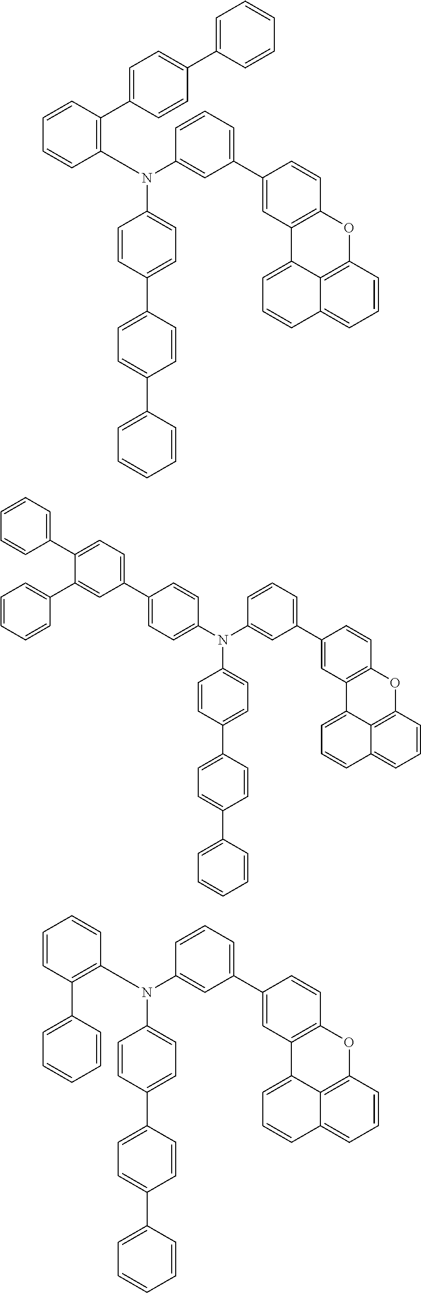

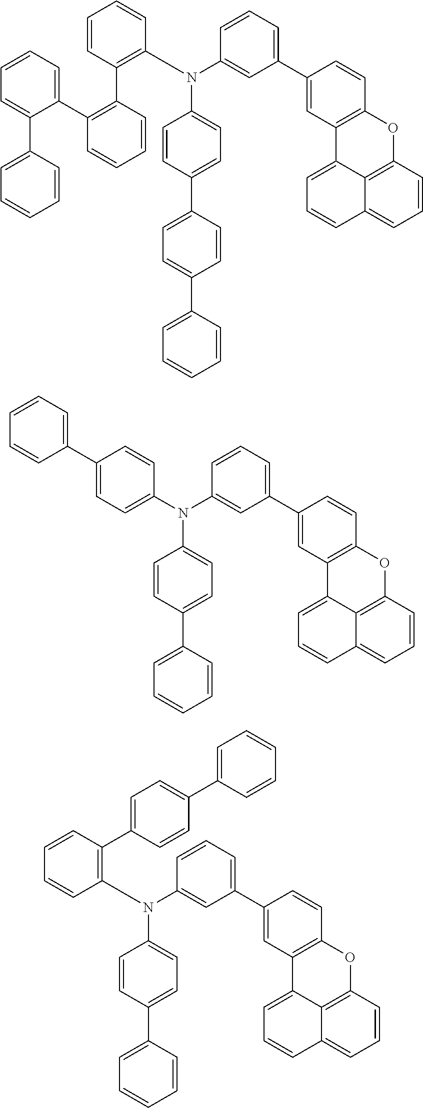 [SMILES: c1ccc(-c2ccc(-c3ccc(N(c4cccc(-c5ccc6c(c5)-c5cccc7cccc(c57)O6)c4)c4ccccc4-c4ccccc4-c4ccccc4-c4ccccc4)cc3)cc2)cc1 ; c1ccc(-c2ccc(N(c3ccc(-c4ccccc4)cc3)c3cccc(-c4ccc5c(c4)-c4cccc6cccc(c46)O5)c3)cc2)cc1 ; c1ccc(-c2ccc(-c3ccccc3N(c3ccc(-c4ccccc4)cc3)c3cccc(-c4ccc5c(c4)-c4cccc6cccc(c46)O5)c3)cc2)cc1]

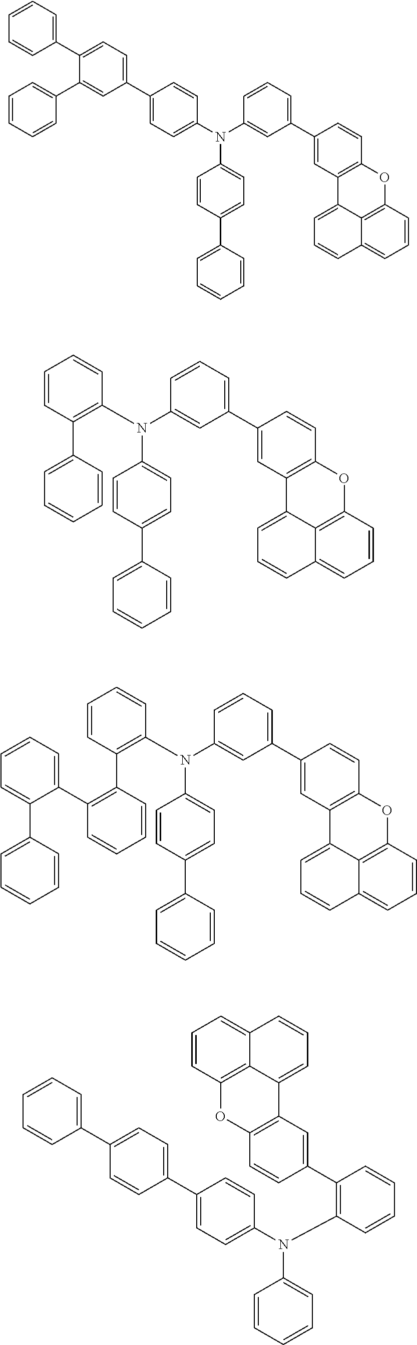 [SMILES: c1ccc(-c2ccc(N(c3ccc(-c4ccc(-c5ccccc5)c(-c5ccccc5)c4)cc3)c3cccc(-c4ccc5c(c4)-c4cccc6cccc(c46)O5)c3)cc2)cc1 ; c1ccc(-c2ccc(N(c3cccc(-c4ccc5c(c4)-c4cccc6cccc(c46)O5)c3)c3ccccc3-c3ccccc3)cc2)cc1 ; c1ccc(-c2ccc(N(c3cccc(-c4ccc5c(c4)-c4cccc6cccc(c46)O5)c3)c3ccccc3-c3ccccc3-c3ccccc3-c3ccccc3)cc2)cc1 ; c1ccc(-c2ccc(-c3ccc(N(c4ccccc4)c4ccccc4-c4ccc5c(c4)-c4cccc6cccc(c46)O5)cc3)cc2)cc1]

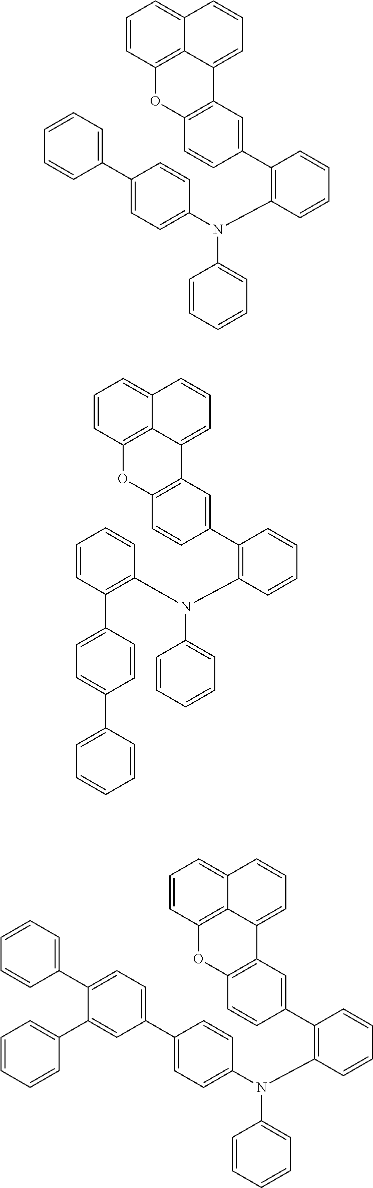 [SMILES: c1ccc(-c2ccc(N(c3ccccc3)c3ccccc3-c3ccc4c(c3)-c3cccc5cccc(c35)O4)cc2)cc1 ; c1ccc(-c2ccc(-c3ccccc3N(c3ccccc3)c3ccccc3-c3ccc4c(c3)-c3cccc5cccc(c35)O4)cc2)cc1 ; c1ccc(-c2ccc(-c3ccc(N(c4ccccc4)c4ccccc4-c4ccc5c(c4)-c4cccc6cccc(c46)O5)cc3)cc2-c2ccccc2)cc1]

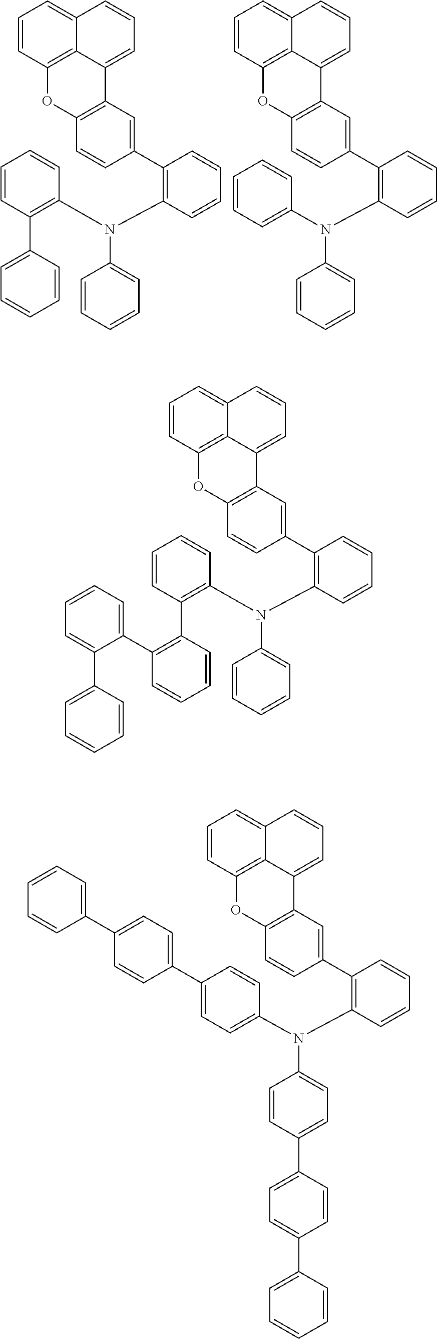

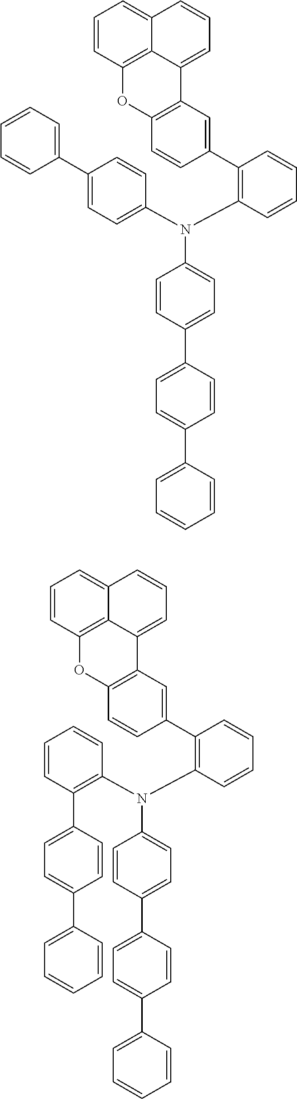 [SMILES: c1ccc(-c2ccc(-c3ccc(N(c4ccc(-c5ccccc5)cc4)c4ccccc4-c4ccc5c(c4)-c4cccc6cccc(c46)O5)cc3)cc2)cc1 ; c1ccc(-c2ccc(-c3ccc(N(c4ccccc4-c4ccc(-c5ccccc5)cc4)c4ccccc4-c4ccc5c(c4)-c4cccc6cccc(c46)O5)cc3)cc2)cc1]

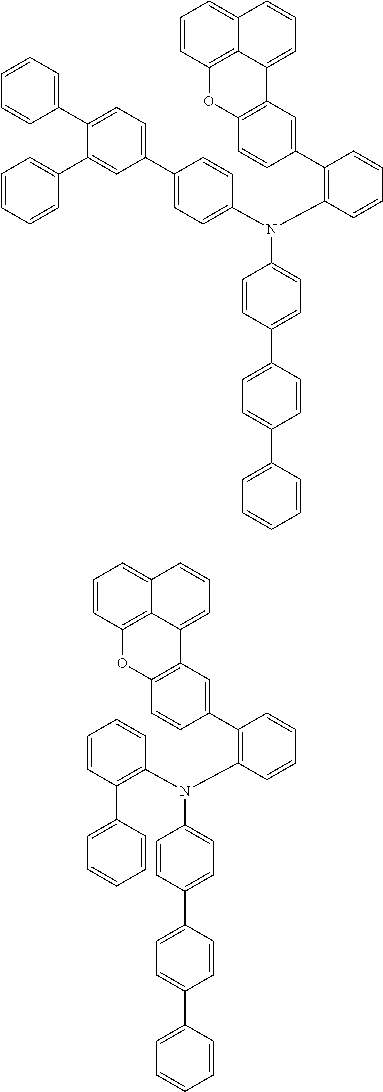 [SMILES: c1ccc(-c2ccc(-c3ccc(N(c4ccc(-c5ccc(-c6ccccc6)c(-c6ccccc6)c5)cc4)c4ccccc4-c4ccc5c(c4)-c4cccc6cccc(c46)O5)cc3)cc2)cc1 ; c1ccc(-c2ccc(-c3ccc(N(c4ccccc4-c4ccccc4)c4ccccc4-c4ccc5c(c4)-c4cccc6cccc(c46)O5)cc3)cc2)cc1]

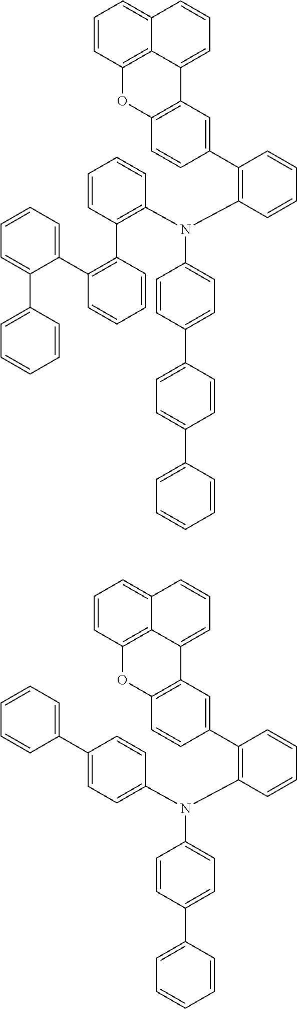 [SMILES: c1ccc(-c2ccc(-c3ccc(N(c4ccccc4-c4ccc5c(c4)-c4cccc6cccc(c46)O5)c4ccccc4-c4ccccc4-c4ccccc4-c4ccccc4)cc3)cc2)cc1 ; c1ccc(-c2ccc(N(c3ccc(-c4ccccc4)cc3)c3ccccc3-c3ccc4c(c3)-c3cccc5cccc(c35)O4)cc2)cc1]

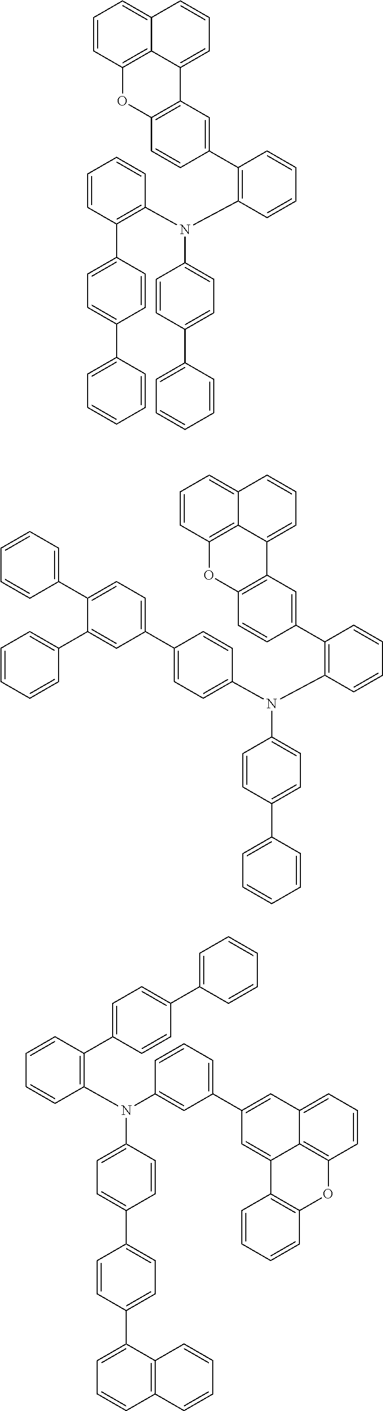 [SMILES: c1ccc(-c2ccc(-c3ccccc3N(c3ccc(-c4ccccc4)cc3)c3ccccc3-c3ccc4c(c3)-c3cccc5cccc(c35)O4)cc2)cc1 ; c1ccc(-c2ccc(N(c3ccc(-c4ccc(-c5ccccc5)c(-c5ccccc5)c4)cc3)c3ccccc3-c3ccc4c(c3)-c3cccc5cccc(c35)O4)cc2)cc1 ; c1ccc(-c2ccc(-c3ccccc3N(c3ccc(-c4ccc(-c5cccc6ccccc56)cc4)cc3)c3cccc(-c4cc5c6c(cccc6c4)Oc4ccccc4-5)c3)cc2)cc1]

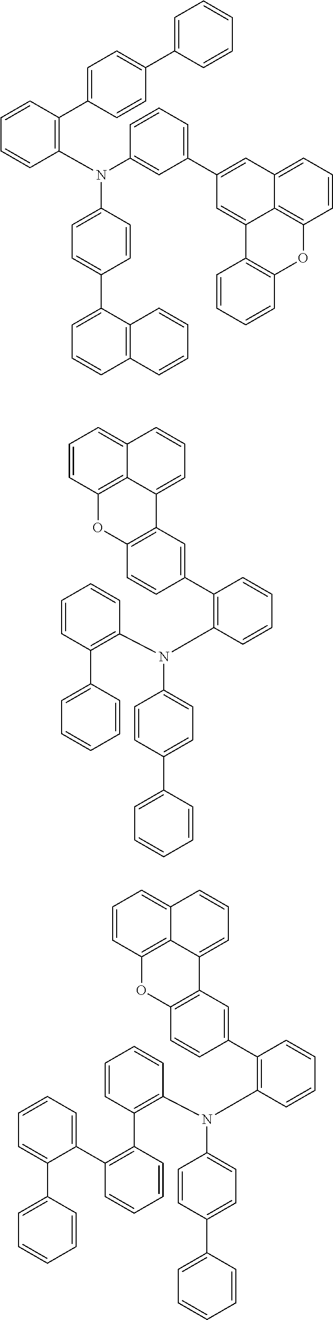

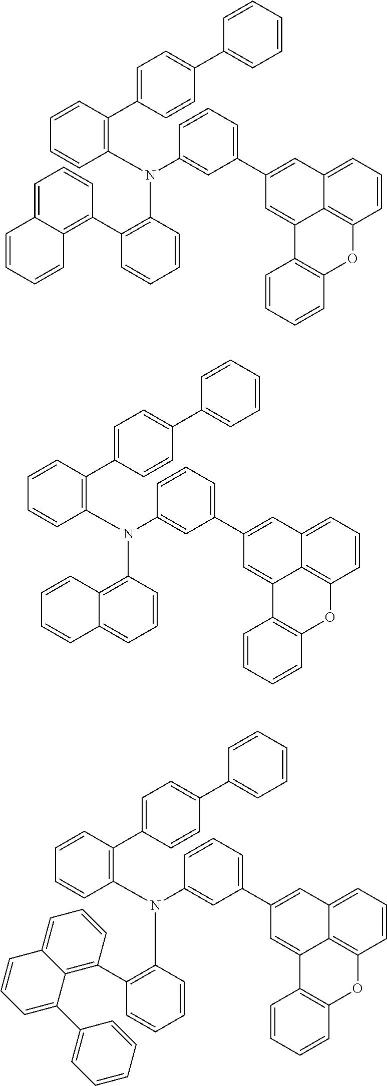 [SMILES: c1ccc(-c2ccc(-c3ccccc3N(c3cccc(-c4cc5c6c(cccc6c4)Oc4ccccc4-5)c3)c3ccccc3-c3cccc4ccccc34)cc2)cc1 ; c1ccc(-c2ccc(-c3ccccc3N(c3cccc(-c4cc5c6c(cccc6c4)Oc4ccccc4-5)c3)c3cccc4ccccc34)cc2)cc1 ; c1ccc(-c2ccc(-c3ccccc3N(c3cccc(-c4cc5c6c(cccc6c4)Oc4ccccc4-5)c3)c3ccccc3-c3cccc4cccc(-c5ccccc5)c34)cc2)cc1]

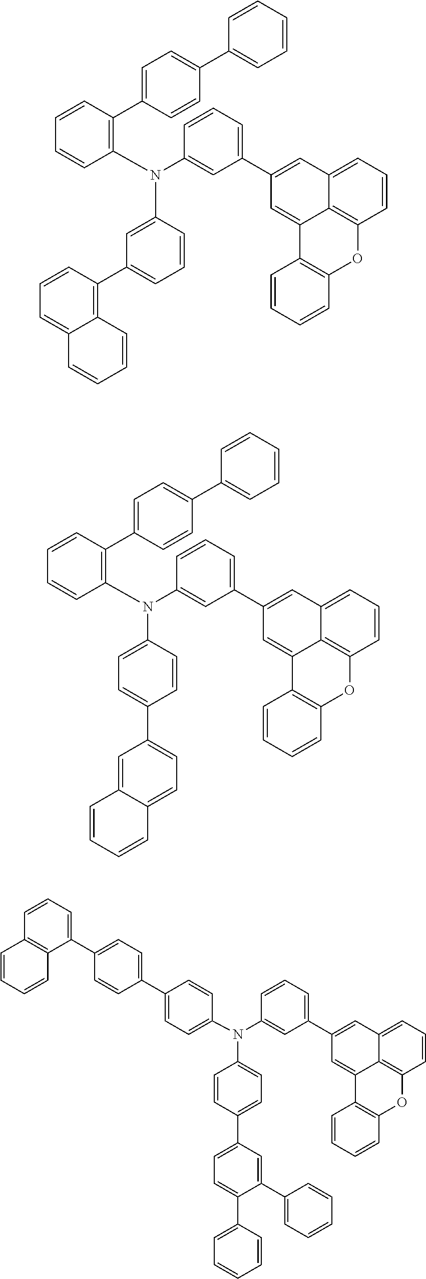 [SMILES: c1ccc(-c2ccc(-c3ccccc3N(c3cccc(-c4cc5c6c(cccc6c4)Oc4ccccc4-5)c3)c3cccc(-c4cccc5ccccc45)c3)cc2)cc1 ; c1ccc(-c2ccc(-c3ccccc3N(c3ccc(-c4ccc5ccccc5c4)cc3)c3cccc(-c4cc5c6c(cccc6c4)Oc4ccccc4-5)c3)cc2)cc1 ; c1ccc(-c2ccc(-c3ccc(N(c4ccc(-c5ccc(-c6cccc7ccccc67)cc5)cc4)c4cccc(-c5cc6c7c(cccc7c5)Oc5ccccc5-6)c4)cc3)cc2-c2ccccc2)cc1]

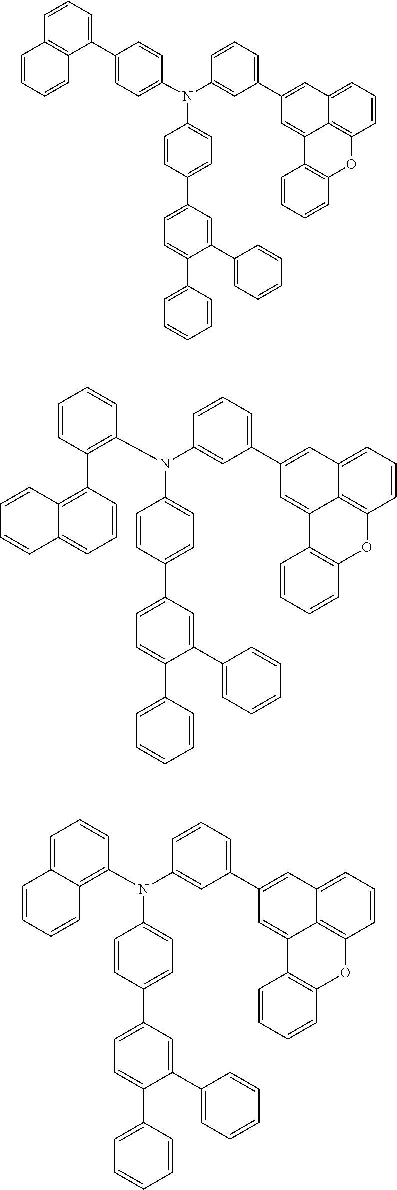 [SMILES: c1ccc(-c2ccc(-c3ccc(N(c4ccc(-c5cccc6ccccc56)cc4)c4cccc(-c5cc6c7c(cccc7c5)Oc5ccccc5-6)c4)cc3)cc2-c2ccccc2)cc1 ; c1ccc(-c2ccc(-c3ccc(N(c4cccc(-c5cc6c7c(cccc7c5)Oc5ccccc5-6)c4)c4ccccc4-c4cccc5ccccc45)cc3)cc2-c2ccccc2)cc1 ; c1ccc(-c2ccc(-c3ccc(N(c4cccc(-c5cc6c7c(cccc7c5)Oc5ccccc5-6)c4)c4cccc5ccccc45)cc3)cc2-c2ccccc2)cc1]

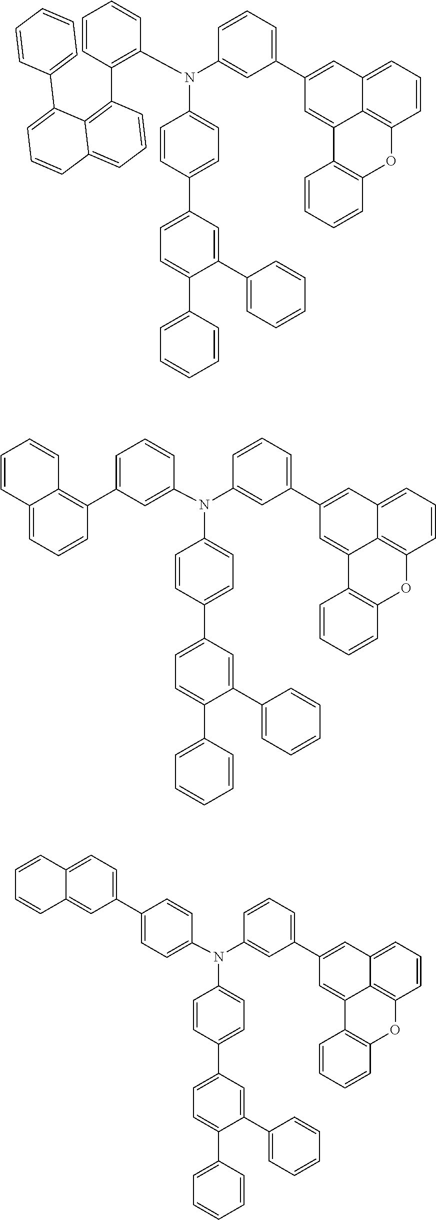 [SMILES: c1ccc(-c2ccc(-c3ccc(N(c4cccc(-c5cc6c7c(cccc7c5)Oc5ccccc5-6)c4)c4ccccc4-c4cccc5cccc(-c6ccccc6)c45)cc3)cc2-c2ccccc2)cc1 ; c1ccc(-c2ccc(-c3ccc(N(c4cccc(-c5cc6c7c(cccc7c5)Oc5ccccc5-6)c4)c4cccc(-c5cccc6ccccc56)c4)cc3)cc2-c2ccccc2)cc1 ; c1ccc(-c2ccc(-c3ccc(N(c4ccc(-c5ccc6ccccc6c5)cc4)c4cccc(-c5cc6c7c(cccc7c5)Oc5ccccc5-6)c4)cc3)cc2-c2ccccc2)cc1]

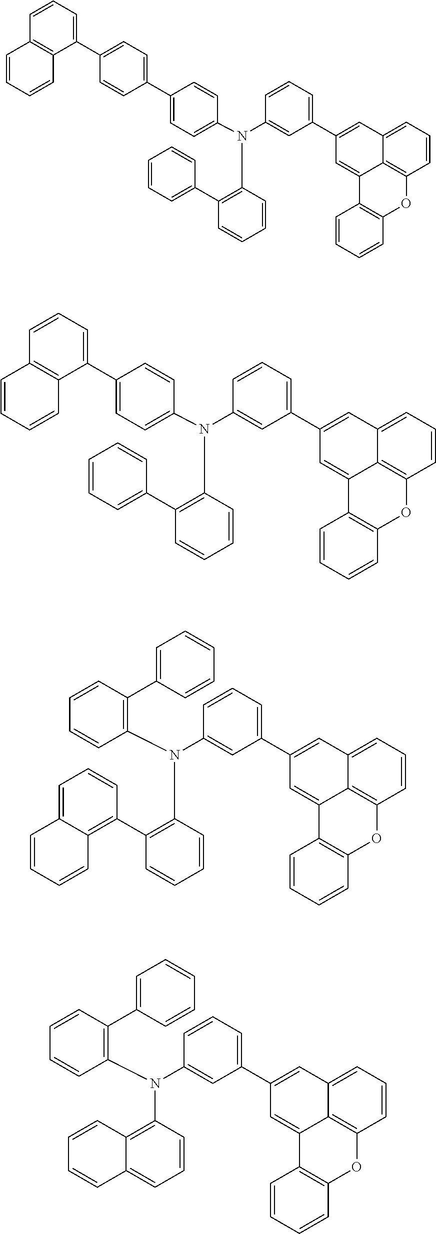 [SMILES: c1ccc(-c2ccccc2N(c2ccc(-c3ccc(-c4cccc5ccccc45)cc3)cc2)c2cccc(-c3cc4c5c(cccc5c3)Oc3ccccc3-4)c2)cc1 ; c1ccc(-c2ccccc2N(c2ccc(-c3cccc4ccccc34)cc2)c2cccc(-c3cc4c5c(cccc5c3)Oc3ccccc3-4)c2)cc1 ; c1ccc(-c2ccccc2N(c2cccc(-c3cc4c5c(cccc5c3)Oc3ccccc3-4)c2)c2ccccc2-c2cccc3ccccc23)cc1 ; c1ccc(-c2ccccc2N(c2cccc(-c3cc4c5c(cccc5c3)Oc3ccccc3-4)c2)c2cccc3ccccc23)cc1]

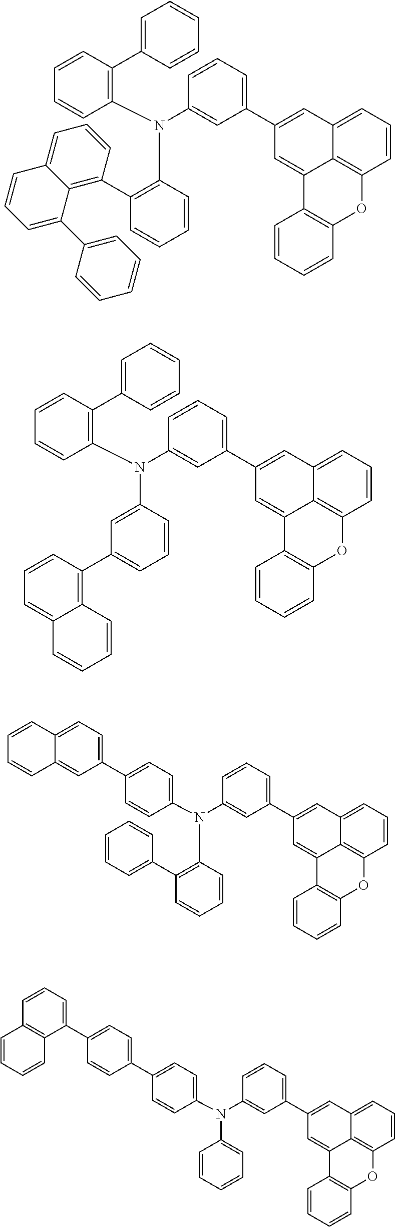 [SMILES: c1ccc(-c2ccccc2N(c2cccc(-c3cc4c5c(cccc5c3)Oc3ccccc3-4)c2)c2ccccc2-c2cccc3cccc(-c4ccccc4)c23)cc1 ; c1ccc(-c2ccccc2N(c2cccc(-c3cc4c5c(cccc5c3)Oc3ccccc3-4)c2)c2cccc(-c3cccc4ccccc34)c2)cc1 ; c1ccc(-c2ccccc2N(c2ccc(-c3ccc4ccccc4c3)cc2)c2cccc(-c3cc4c5c(cccc5c3)Oc3ccccc3-4)c2)cc1 ; c1ccc(N(c2ccc(-c3ccc(-c4cccc5ccccc45)cc3)cc2)c2cccc(-c3cc4c5c(cccc5c3)Oc3ccccc3-4)c2)cc1]

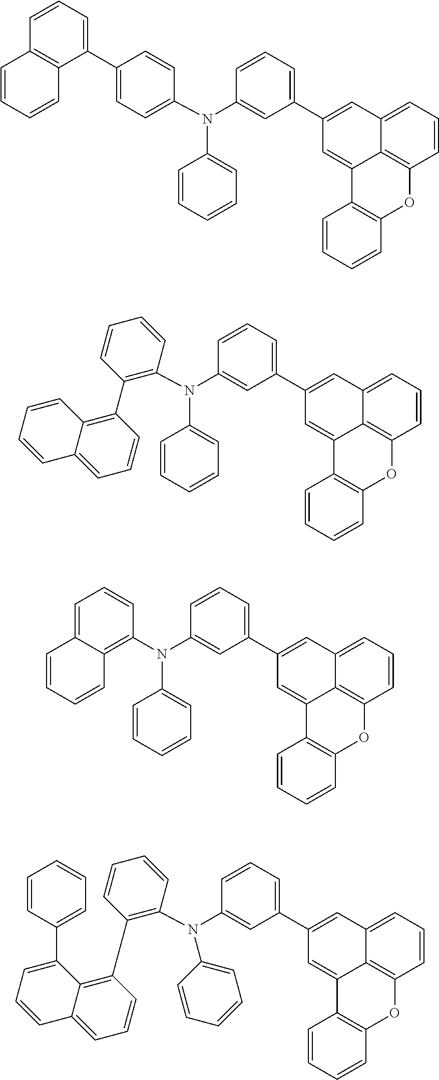 [SMILES: c1ccc(N(c2ccc(-c3cccc4ccccc34)cc2)c2cccc(-c3cc4c5c(cccc5c3)Oc3ccccc3-4)c2)cc1 ; c1ccc(N(c2cccc(-c3cc4c5c(cccc5c3)Oc3ccccc3-4)c2)c2ccccc2-c2cccc3ccccc23)cc1 ; c1ccc(N(c2cccc(-c3cc4c5c(cccc5c3)Oc3ccccc3-4)c2)c2cccc3ccccc23)cc1 ; c1ccc(-c2cccc3cccc(-c4ccccc4N(c4ccccc4)c4cccc(-c5cc6c7c(cccc7c5)Oc5ccccc5-6)c4)c23)cc1]

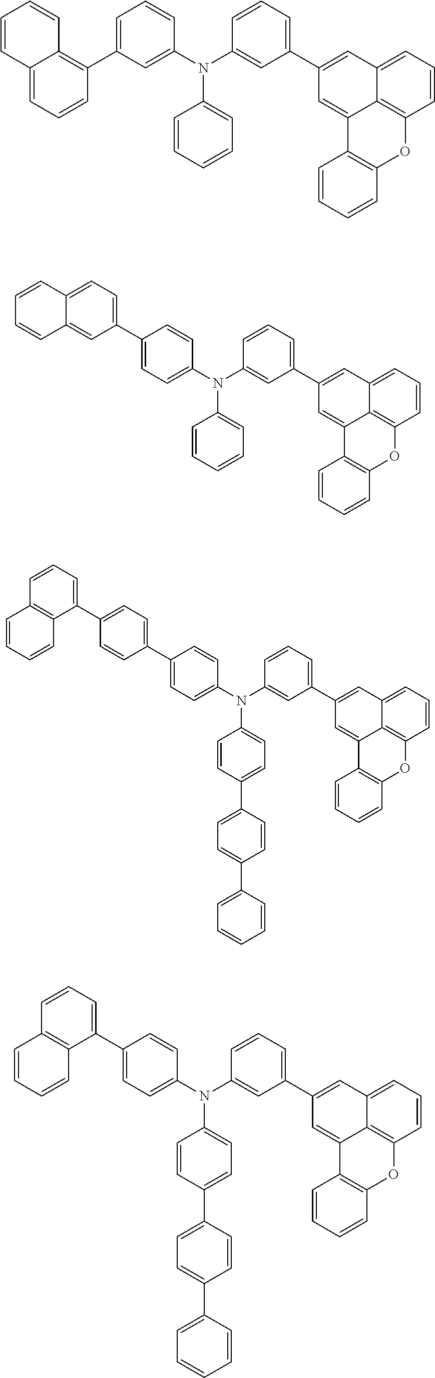 [SMILES: c1ccc(N(c2cccc(-c3cc4c5c(cccc5c3)Oc3ccccc3-4)c2)c2cccc(-c3cccc4ccccc34)c2)cc1 ; c1ccc(N(c2ccc(-c3ccc4ccccc4c3)cc2)c2cccc(-c3cc4c5c(cccc5c3)Oc3ccccc3-4)c2)cc1 ; c1ccc(-c2ccc(-c3ccc(N(c4ccc(-c5ccc(-c6cccc7ccccc67)cc5)cc4)c4cccc(-c5cc6c7c(cccc7c5)Oc5ccccc5-6)c4)cc3)cc2)cc1 ; c1ccc(-c2ccc(-c3ccc(N(c4ccc(-c5cccc6ccccc56)cc4)c4cccc(-c5cc6c7c(cccc7c5)Oc5ccccc5-6)c4)cc3)cc2)cc1]

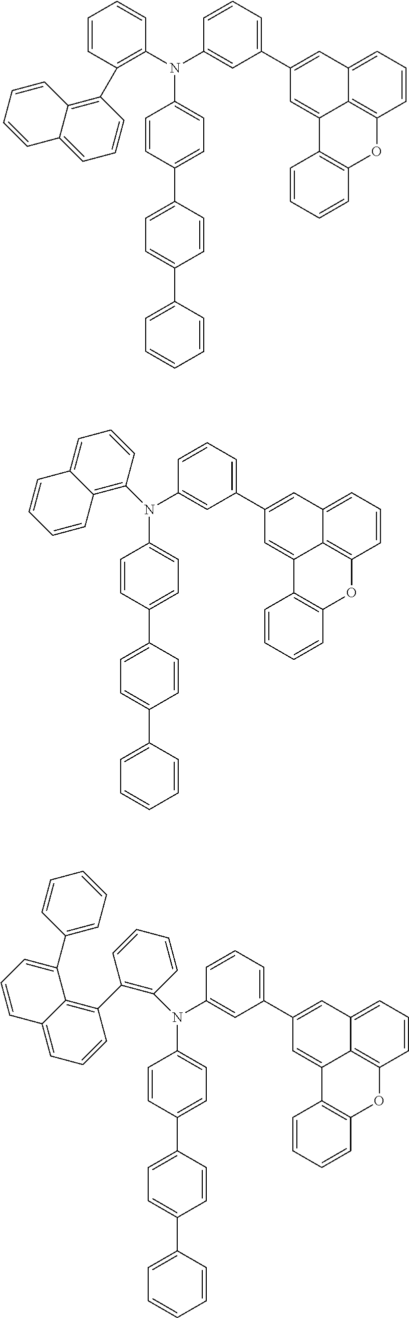 [SMILES: c1ccc(-c2ccc(-c3ccc(N(c4cccc(-c5cc6c7c(cccc7c5)Oc5ccccc5-6)c4)c4ccccc4-c4cccc5ccccc45)cc3)cc2)cc1 ; c1ccc(-c2ccc(-c3ccc(N(c4cccc(-c5cc6c7c(cccc7c5)Oc5ccccc5-6)c4)c4cccc5ccccc45)cc3)cc2)cc1 ; c1ccc(-c2ccc(-c3ccc(N(c4cccc(-c5cc6c7c(cccc7c5)Oc5ccccc5-6)c4)c4ccccc4-c4cccc5cccc(-c6ccccc6)c45)cc3)cc2)cc1]

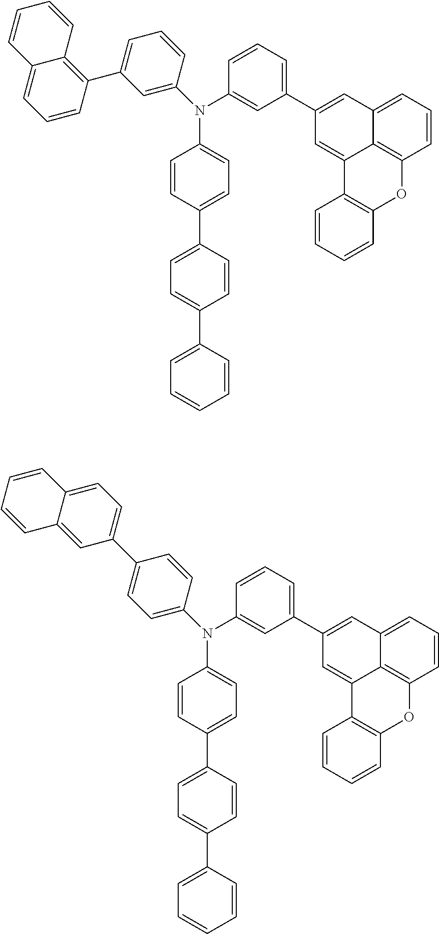 [SMILES: c1ccc(-c2ccc(-c3ccc(N(c4cccc(-c5cc6c7c(cccc7c5)Oc5ccccc5-6)c4)c4cccc(-c5cccc6ccccc56)c4)cc3)cc2)cc1 ; c1ccc(-c2ccc(-c3ccc(N(c4ccc(-c5ccc6ccccc6c5)cc4)c4cccc(-c5cc6c7c(cccc7c5)Oc5ccccc5-6)c4)cc3)cc2)cc1]

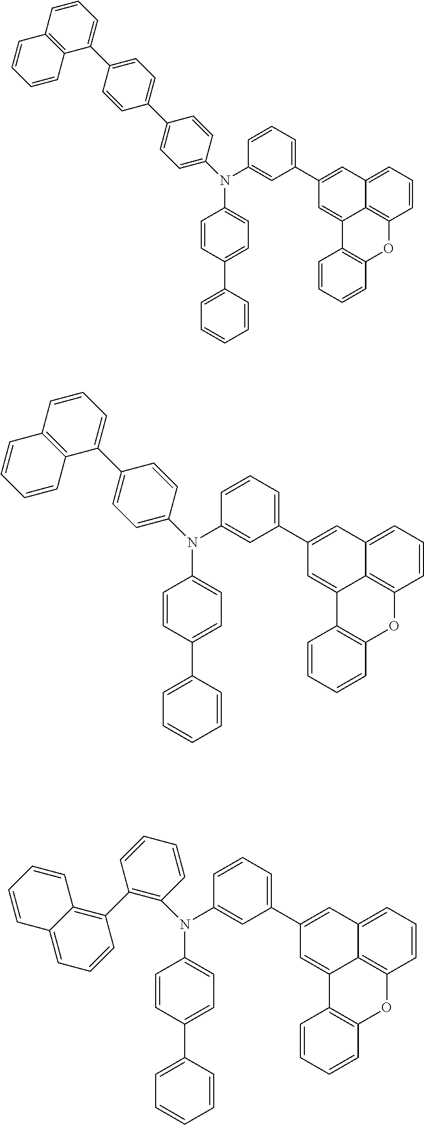 [SMILES: c1ccc(-c2ccc(N(c3ccc(-c4ccc(-c5cccc6ccccc56)cc4)cc3)c3cccc(-c4cc5c6c(cccc6c4)Oc4ccccc4-5)c3)cc2)cc1 ; c1ccc(-c2ccc(N(c3ccc(-c4cccc5ccccc45)cc3)c3cccc(-c4cc5c6c(cccc6c4)Oc4ccccc4-5)c3)cc2)cc1 ; c1ccc(-c2ccc(N(c3cccc(-c4cc5c6c(cccc6c4)Oc4ccccc4-5)c3)c3ccccc3-c3cccc4ccccc34)cc2)cc1]

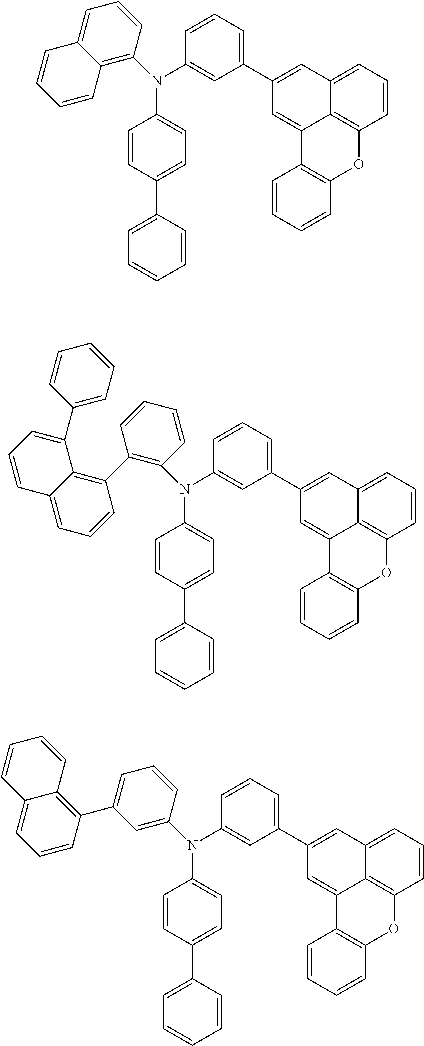 [SMILES: c1ccc(-c2ccc(N(c3cccc(-c4cc5c6c(cccc6c4)Oc4ccccc4-5)c3)c3cccc4ccccc34)cc2)cc1 ; c1ccc(-c2ccc(N(c3cccc(-c4cc5c6c(cccc6c4)Oc4ccccc4-5)c3)c3ccccc3-c3cccc4cccc(-c5ccccc5)c34)cc2)cc1 ; c1ccc(-c2ccc(N(c3cccc(-c4cc5c6c(cccc6c4)Oc4ccccc4-5)c3)c3cccc(-c4cccc5ccccc45)c3)cc2)cc1]

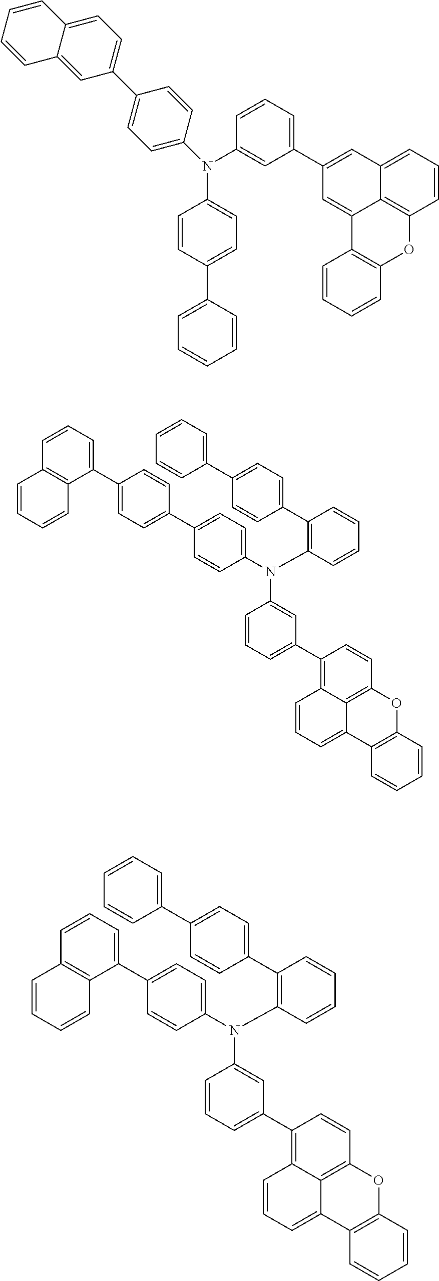 [SMILES: c1ccc(-c2ccc(N(c3ccc(-c4ccc5ccccc5c4)cc3)c3cccc(-c4cc5c6c(cccc6c4)Oc4ccccc4-5)c3)cc2)cc1 ; c1ccc(-c2ccc(-c3ccccc3N(c3ccc(-c4ccc(-c5cccc6ccccc56)cc4)cc3)c3cccc(-c4ccc5c6c(cccc46)-c4ccccc4O5)c3)cc2)cc1 ; c1ccc(-c2ccc(-c3ccccc3N(c3ccc(-c4cccc5ccccc45)cc3)c3cccc(-c4ccc5c6c(cccc46)-c4ccccc4O5)c3)cc2)cc1]

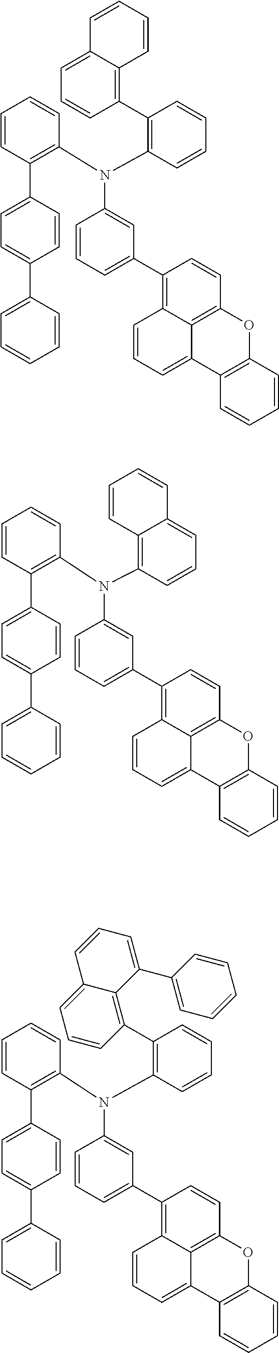 [SMILES: c1ccc(-c2ccc(-c3ccccc3N(c3cccc(-c4ccc5c6c(cccc46)-c4ccccc4O5)c3)c3ccccc3-c3cccc4ccccc34)cc2)cc1 ; c1ccc(-c2ccc(-c3ccccc3N(c3cccc(-c4ccc5c6c(cccc46)-c4ccccc4O5)c3)c3cccc4ccccc34)cc2)cc1 ; c1ccc(-c2ccc(-c3ccccc3N(c3cccc(-c4ccc5c6c(cccc46)-c4ccccc4O5)c3)c3ccccc3-c3cccc4cccc(-c5ccccc5)c34)cc2)cc1]

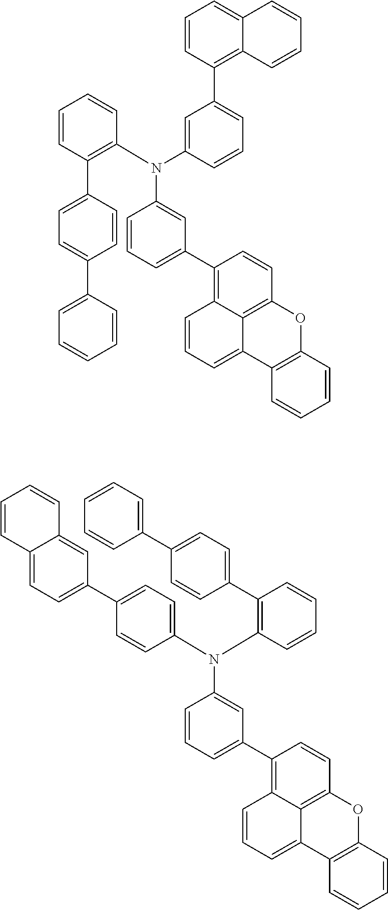 [SMILES: c1ccc(-c2ccc(-c3ccccc3N(c3cccc(-c4cccc5ccccc45)c3)c3cccc(-c4ccc5c6c(cccc46)-c4ccccc4O5)c3)cc2)cc1 ; c1ccc(-c2ccc(-c3ccccc3N(c3ccc(-c4ccc5ccccc5c4)cc3)c3cccc(-c4ccc5c6c(cccc46)-c4ccccc4O5)c3)cc2)cc1]

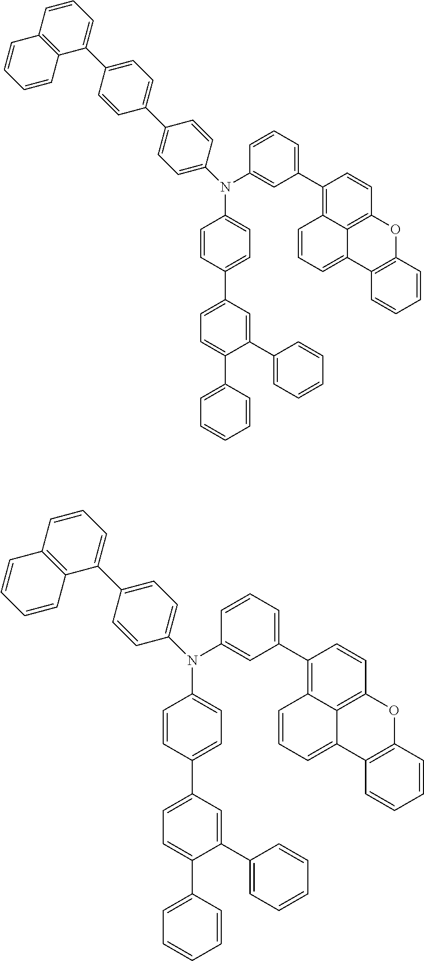 [SMILES: c1ccc(-c2ccc(-c3ccc(N(c4ccc(-c5ccc(-c6cccc7ccccc67)cc5)cc4)c4cccc(-c5ccc6c7c(cccc57)-c5ccccc5O6)c4)cc3)cc2-c2ccccc2)cc1 ; c1ccc(-c2ccc(-c3ccc(N(c4ccc(-c5cccc6ccccc56)cc4)c4cccc(-c5ccc6c7c(cccc57)-c5ccccc5O6)c4)cc3)cc2-c2ccccc2)cc1]

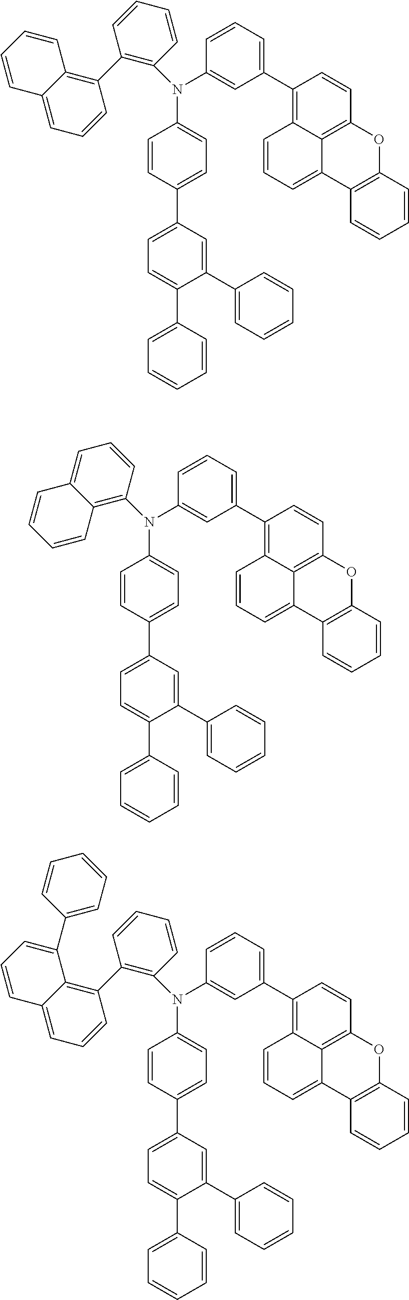 [SMILES: c1ccc(-c2ccc(-c3ccc(N(c4cccc(-c5ccc6c7c(cccc57)-c5ccccc5O6)c4)c4ccccc4-c4cccc5ccccc45)cc3)cc2-c2ccccc2)cc1 ; c1ccc(-c2ccc(-c3ccc(N(c4cccc(-c5ccc6c7c(cccc57)-c5ccccc5O6)c4)c4cccc5ccccc45)cc3)cc2-c2ccccc2)cc1 ; c1ccc(-c2ccc(-c3ccc(N(c4cccc(-c5ccc6c7c(cccc57)-c5ccccc5O6)c4)c4ccccc4-c4cccc5cccc(-c6ccccc6)c45)cc3)cc2-c2ccccc2)cc1]

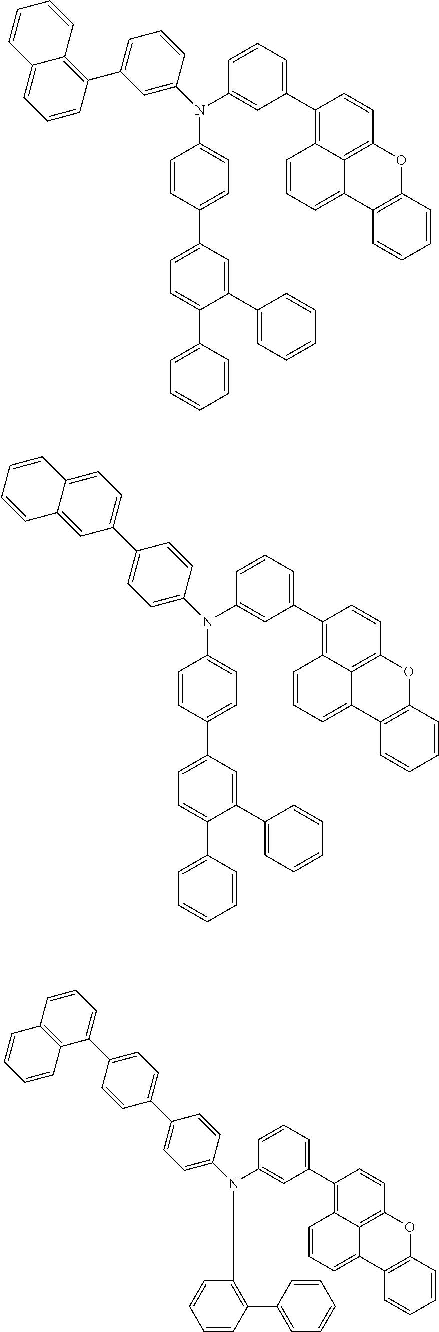 [SMILES: c1ccc(-c2ccc(-c3ccc(N(c4cccc(-c5cccc6ccccc56)c4)c4cccc(-c5ccc6c7c(cccc57)-c5ccccc5O6)c4)cc3)cc2-c2ccccc2)cc1 ; c1ccc(-c2ccc(-c3ccc(N(c4ccc(-c5ccc6ccccc6c5)cc4)c4cccc(-c5ccc6c7c(cccc57)-c5ccccc5O6)c4)cc3)cc2-c2ccccc2)cc1 ; c1ccc(-c2ccccc2N(c2ccc(-c3ccc(-c4cccc5ccccc45)cc3)cc2)c2cccc(-c3ccc4c5c(cccc35)-c3ccccc3O4)c2)cc1]

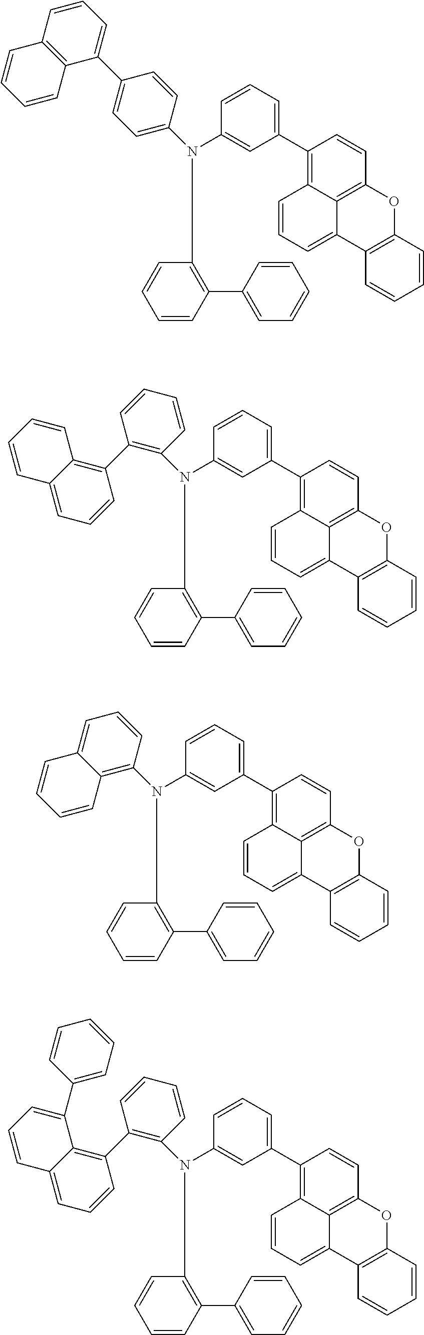 [SMILES: c1ccc(-c2ccccc2N(c2ccc(-c3cccc4ccccc34)cc2)c2cccc(-c3ccc4c5c(cccc35)-c3ccccc3O4)c2)cc1 ; c1ccc(-c2ccccc2N(c2cccc(-c3ccc4c5c(cccc35)-c3ccccc3O4)c2)c2ccccc2-c2cccc3ccccc23)cc1 ; c1ccc(-c2ccccc2N(c2cccc(-c3ccc4c5c(cccc35)-c3ccccc3O4)c2)c2cccc3ccccc23)cc1 ; c1ccc(-c2ccccc2N(c2cccc(-c3ccc4c5c(cccc35)-c3ccccc3O4)c2)c2ccccc2-c2cccc3cccc(-c4ccccc4)c23)cc1]

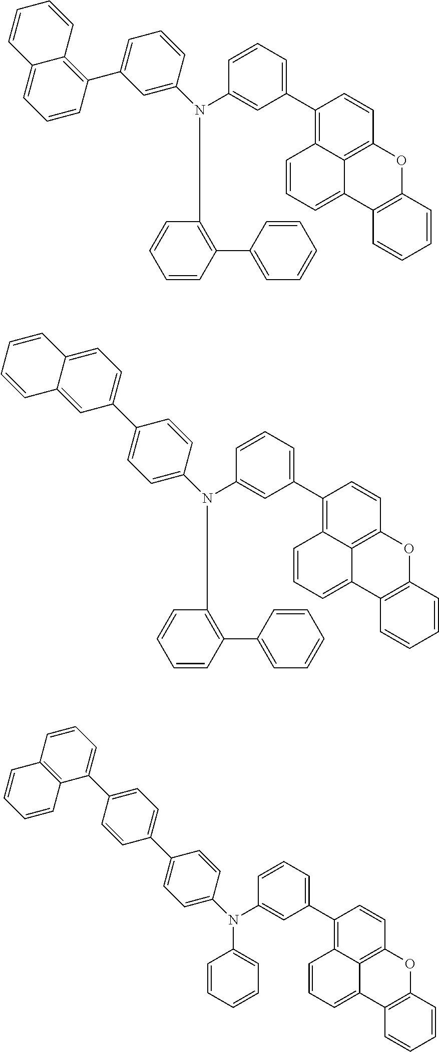 [SMILES: c1ccc(-c2ccccc2N(c2cccc(-c3cccc4ccccc34)c2)c2cccc(-c3ccc4c5c(cccc35)-c3ccccc3O4)c2)cc1 ; c1ccc(-c2ccccc2N(c2ccc(-c3ccc4ccccc4c3)cc2)c2cccc(-c3ccc4c5c(cccc35)-c3ccccc3O4)c2)cc1 ; c1ccc(N(c2ccc(-c3ccc(-c4cccc5ccccc45)cc3)cc2)c2cccc(-c3ccc4c5c(cccc35)-c3ccccc3O4)c2)cc1]

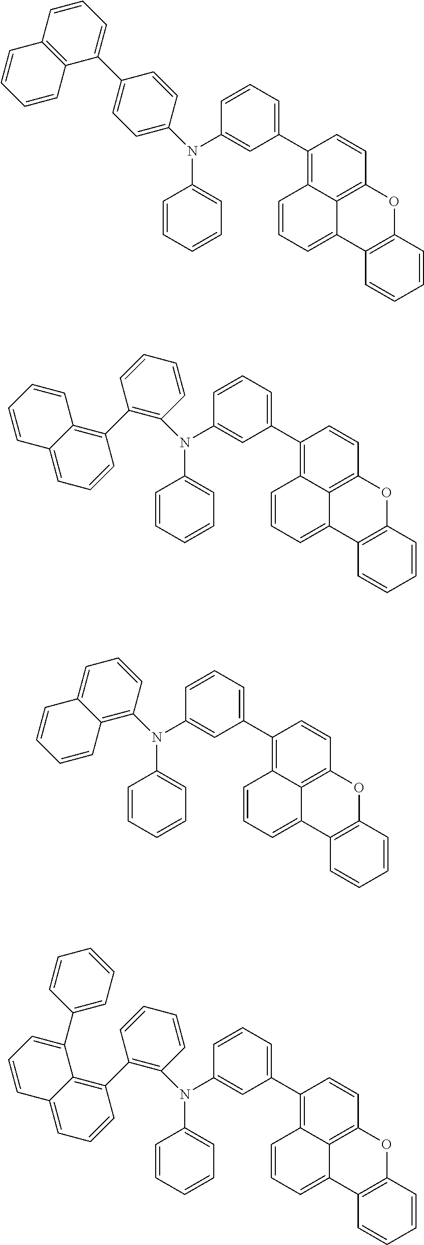 [SMILES: c1ccc(N(c2ccc(-c3cccc4ccccc34)cc2)c2cccc(-c3ccc4c5c(cccc35)-c3ccccc3O4)c2)cc1 ; c1ccc(N(c2cccc(-c3ccc4c5c(cccc35)-c3ccccc3O4)c2)c2ccccc2-c2cccc3ccccc23)cc1 ; c1ccc(N(c2cccc(-c3ccc4c5c(cccc35)-c3ccccc3O4)c2)c2cccc3ccccc23)cc1 ; c1ccc(-c2cccc3cccc(-c4ccccc4N(c4ccccc4)c4cccc(-c5ccc6c7c(cccc57)-c5ccccc5O6)c4)c23)cc1]

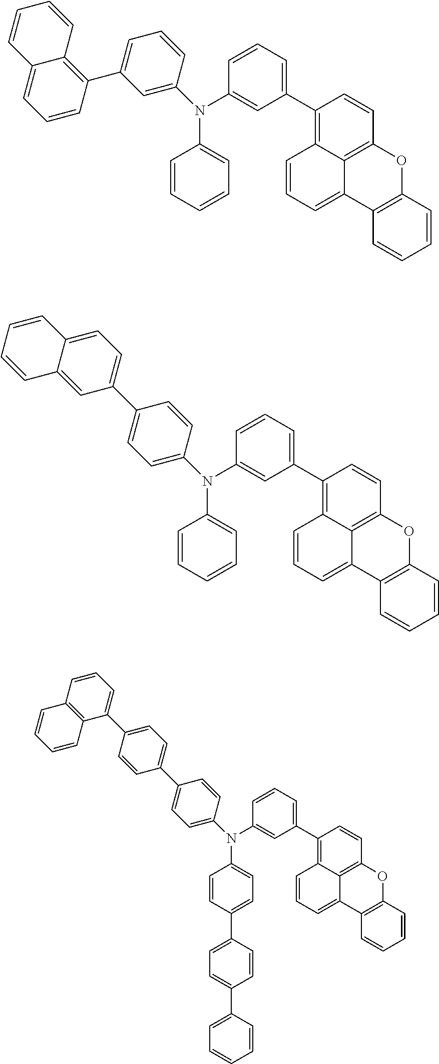 [SMILES: c1ccc(N(c2cccc(-c3cccc4ccccc34)c2)c2cccc(-c3ccc4c5c(cccc35)-c3ccccc3O4)c2)cc1 ; c1ccc(N(c2ccc(-c3ccc4ccccc4c3)cc2)c2cccc(-c3ccc4c5c(cccc35)-c3ccccc3O4)c2)cc1 ; c1ccc(-c2ccc(-c3ccc(N(c4ccc(-c5ccc(-c6cccc7ccccc67)cc5)cc4)c4cccc(-c5ccc6c7c(cccc57)-c5ccccc5O6)c4)cc3)cc2)cc1]

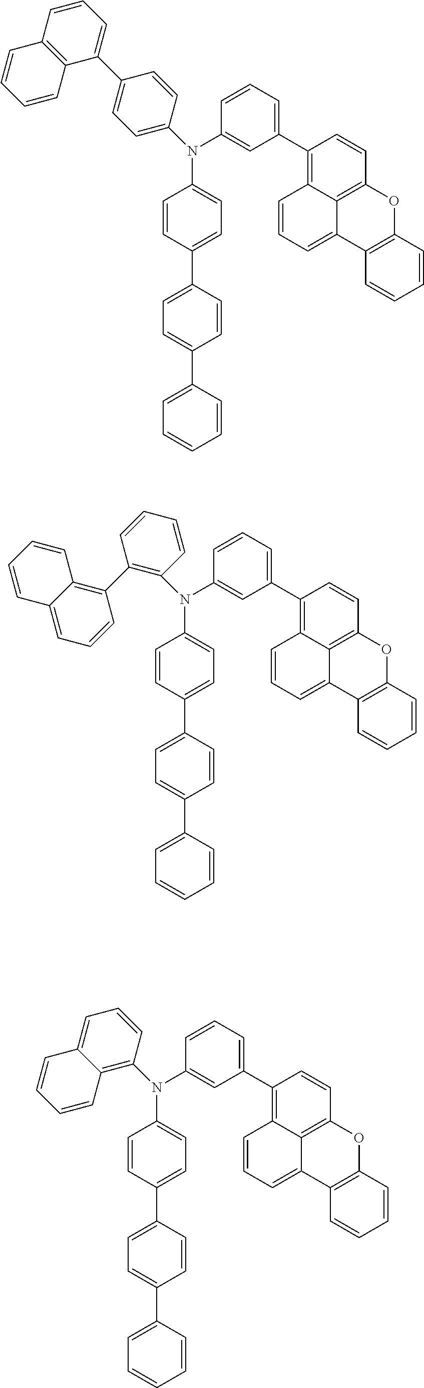 [SMILES: c1ccc(-c2ccc(-c3ccc(N(c4ccc(-c5cccc6ccccc56)cc4)c4cccc(-c5ccc6c7c(cccc57)-c5ccccc5O6)c4)cc3)cc2)cc1 ; c1ccc(-c2ccc(-c3ccc(N(c4cccc(-c5ccc6c7c(cccc57)-c5ccccc5O6)c4)c4ccccc4-c4cccc5ccccc45)cc3)cc2)cc1 ; c1ccc(-c2ccc(-c3ccc(N(c4cccc(-c5ccc6c7c(cccc57)-c5ccccc5O6)c4)c4cccc5ccccc45)cc3)cc2)cc1]

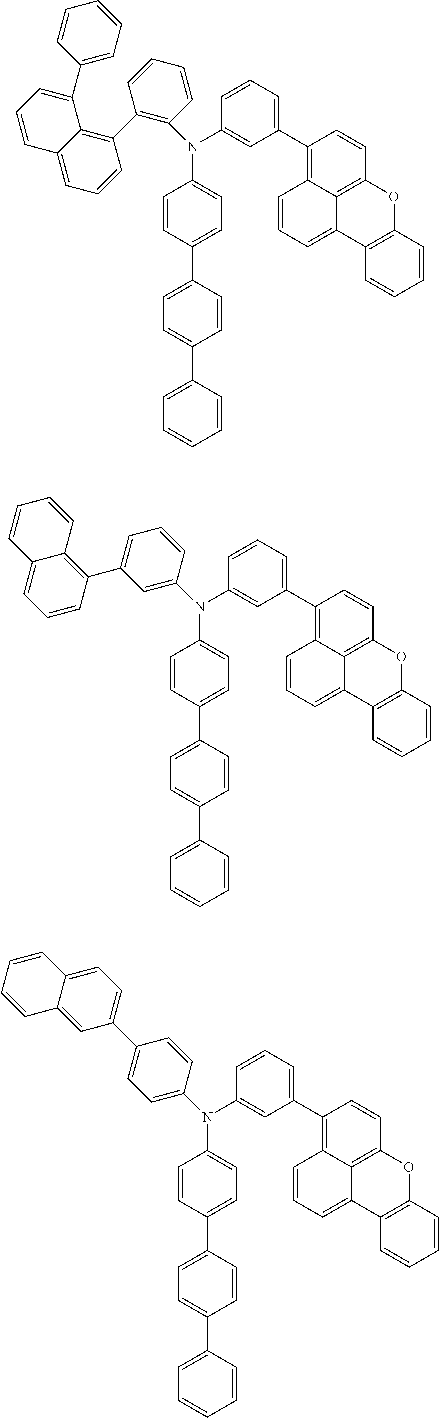 [SMILES: c1ccc(-c2ccc(-c3ccc(N(c4cccc(-c5ccc6c7c(cccc57)-c5ccccc5O6)c4)c4ccccc4-c4cccc5cccc(-c6ccccc6)c45)cc3)cc2)cc1 ; c1ccc(-c2ccc(-c3ccc(N(c4cccc(-c5cccc6ccccc56)c4)c4cccc(-c5ccc6c7c(cccc57)-c5ccccc5O6)c4)cc3)cc2)cc1 ; c1ccc(-c2ccc(-c3ccc(N(c4ccc(-c5ccc6ccccc6c5)cc4)c4cccc(-c5ccc6c7c(cccc57)-c5ccccc5O6)c4)cc3)cc2)cc1]

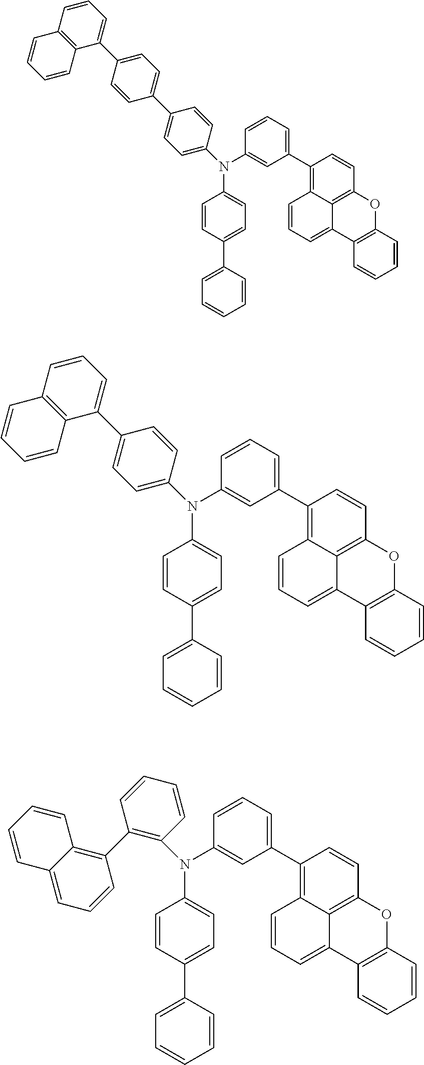 [SMILES: c1ccc(-c2ccc(N(c3ccc(-c4ccc(-c5cccc6ccccc56)cc4)cc3)c3cccc(-c4ccc5c6c(cccc46)-c4ccccc4O5)c3)cc2)cc1 ; c1ccc(-c2ccc(N(c3ccc(-c4cccc5ccccc45)cc3)c3cccc(-c4ccc5c6c(cccc46)-c4ccccc4O5)c3)cc2)cc1 ; c1ccc(-c2ccc(N(c3cccc(-c4ccc5c6c(cccc46)-c4ccccc4O5)c3)c3ccccc3-c3cccc4ccccc34)cc2)cc1]

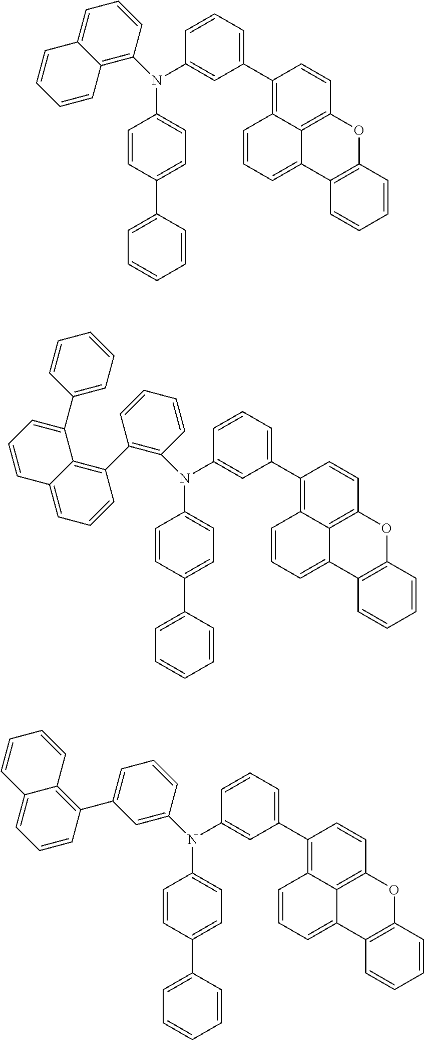 [SMILES: c1ccc(-c2ccc(N(c3cccc(-c4ccc5c6c(cccc46)-c4ccccc4O5)c3)c3cccc4ccccc34)cc2)cc1 ; c1ccc(-c2ccc(N(c3cccc(-c4ccc5c6c(cccc46)-c4ccccc4O5)c3)c3ccccc3-c3cccc4cccc(-c5ccccc5)c34)cc2)cc1 ; c1ccc(-c2ccc(N(c3cccc(-c4cccc5ccccc45)c3)c3cccc(-c4ccc5c6c(cccc46)-c4ccccc4O5)c3)cc2)cc1]

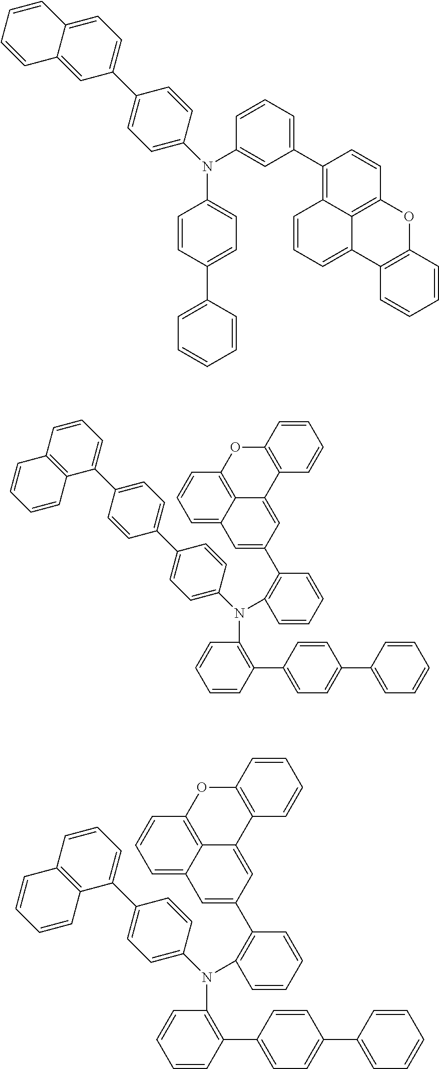 [SMILES: c1ccc(-c2ccc(N(c3ccc(-c4ccc5ccccc5c4)cc3)c3cccc(-c4ccc5c6c(cccc46)-c4ccccc4O5)c3)cc2)cc1 ; c1ccc(-c2ccc(-c3ccccc3N(c3ccc(-c4ccc(-c5cccc6ccccc56)cc4)cc3)c3ccccc3-c3cc4c5c(cccc5c3)Oc3ccccc3-4)cc2)cc1 ; c1ccc(-c2ccc(-c3ccccc3N(c3ccc(-c4cccc5ccccc45)cc3)c3ccccc3-c3cc4c5c(cccc5c3)Oc3ccccc3-4)cc2)cc1]

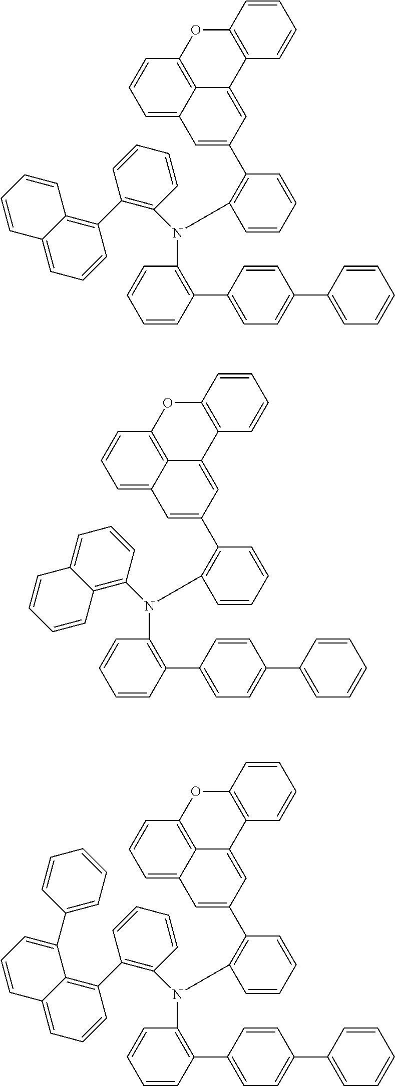 [SMILES: c1ccc(-c2ccc(-c3ccccc3N(c3ccccc3-c3cc4c5c(cccc5c3)Oc3ccccc3-4)c3ccccc3-c3cccc4ccccc34)cc2)cc1 ; c1ccc(-c2ccc(-c3ccccc3N(c3ccccc3-c3cc4c5c(cccc5c3)Oc3ccccc3-4)c3cccc4ccccc34)cc2)cc1 ; c1ccc(-c2ccc(-c3ccccc3N(c3ccccc3-c3cc4c5c(cccc5c3)Oc3ccccc3-4)c3ccccc3-c3cccc4cccc(-c5ccccc5)c34)cc2)cc1]

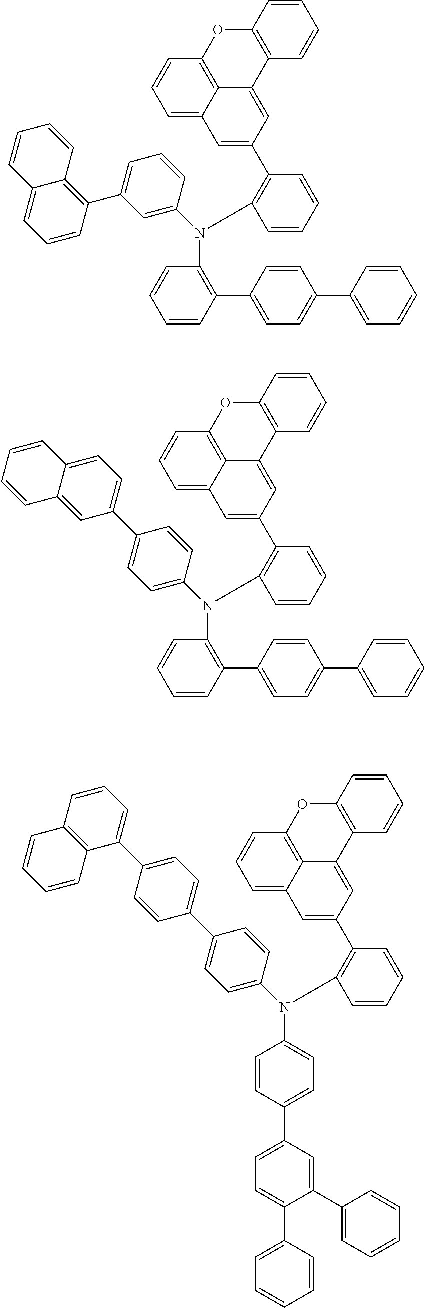 [SMILES: c1ccc(-c2ccc(-c3ccccc3N(c3cccc(-c4cccc5ccccc45)c3)c3ccccc3-c3cc4c5c(cccc5c3)Oc3ccccc3-4)cc2)cc1 ; c1ccc(-c2ccc(-c3ccccc3N(c3ccc(-c4ccc5ccccc5c4)cc3)c3ccccc3-c3cc4c5c(cccc5c3)Oc3ccccc3-4)cc2)cc1 ; c1ccc(-c2ccc(-c3ccc(N(c4ccc(-c5ccc(-c6cccc7ccccc67)cc5)cc4)c4ccccc4-c4cc5c6c(cccc6c4)Oc4ccccc4-5)cc3)cc2-c2ccccc2)cc1]

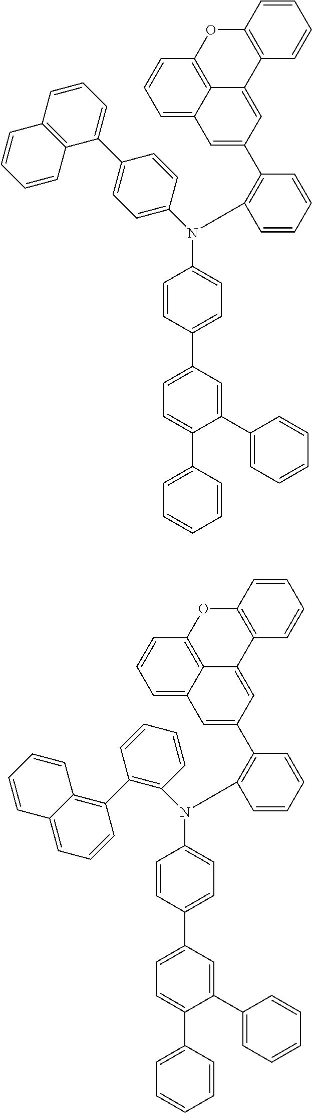 [SMILES: c1ccc(-c2ccc(-c3ccc(N(c4ccc(-c5cccc6ccccc56)cc4)c4ccccc4-c4cc5c6c(cccc6c4)Oc4ccccc4-5)cc3)cc2-c2ccccc2)cc1 ; c1ccc(-c2ccc(-c3ccc(N(c4ccccc4-c4cc5c6c(cccc6c4)Oc4ccccc4-5)c4ccccc4-c4cccc5ccccc45)cc3)cc2-c2ccccc2)cc1]

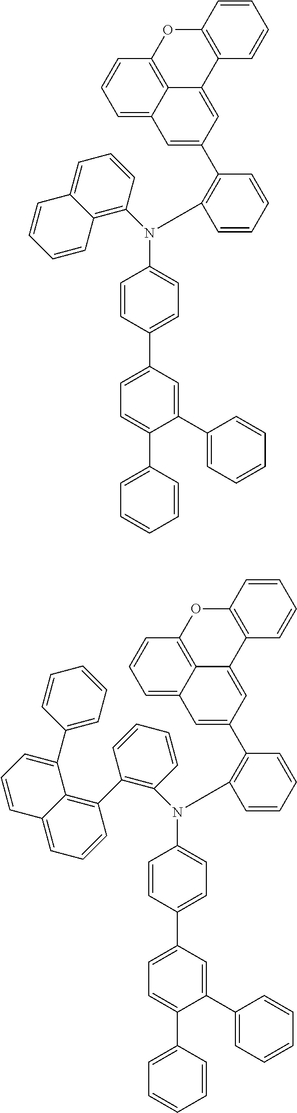 [SMILES: c1ccc(-c2ccc(-c3ccc(N(c4ccccc4-c4cc5c6c(cccc6c4)Oc4ccccc4-5)c4cccc5ccccc45)cc3)cc2-c2ccccc2)cc1 ; c1ccc(-c2ccc(-c3ccc(N(c4ccccc4-c4cc5c6c(cccc6c4)Oc4ccccc4-5)c4ccccc4-c4cccc5cccc(-c6ccccc6)c45)cc3)cc2-c2ccccc2)cc1]

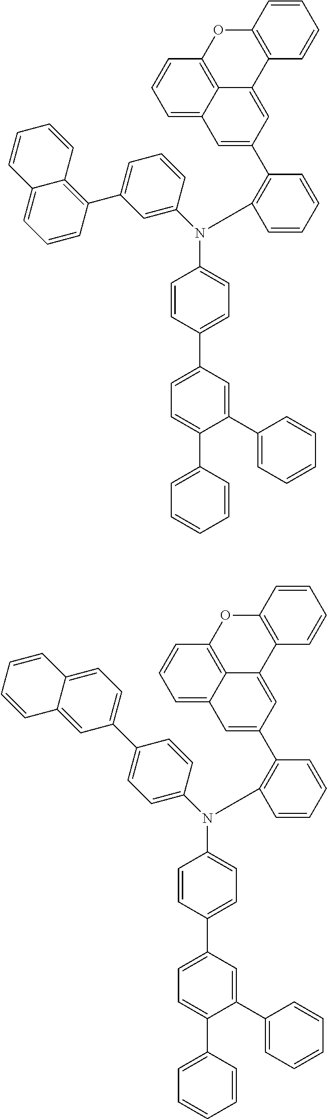 [SMILES: c1ccc(-c2ccc(-c3ccc(N(c4cccc(-c5cccc6ccccc56)c4)c4ccccc4-c4cc5c6c(cccc6c4)Oc4ccccc4-5)cc3)cc2-c2ccccc2)cc1 ; c1ccc(-c2ccc(-c3ccc(N(c4ccc(-c5ccc6ccccc6c5)cc4)c4ccccc4-c4cc5c6c(cccc6c4)Oc4ccccc4-5)cc3)cc2-c2ccccc2)cc1]

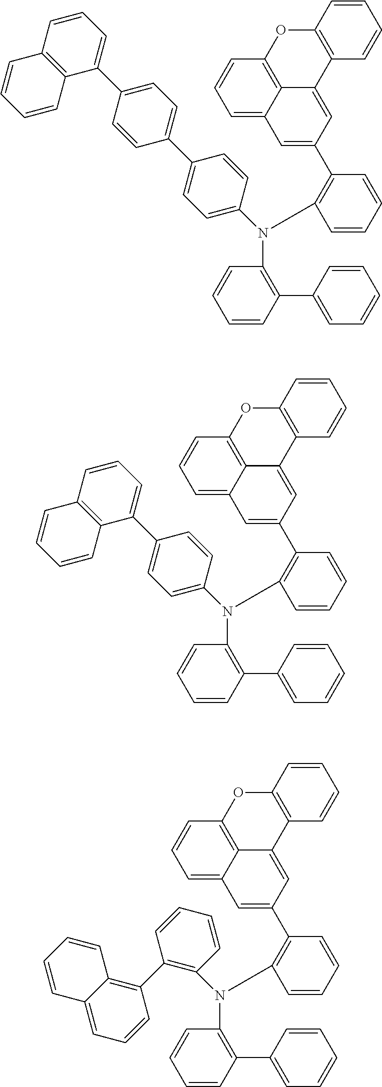 [SMILES: c1ccc(-c2ccccc2N(c2ccc(-c3ccc(-c4cccc5ccccc45)cc3)cc2)c2ccccc2-c2cc3c4c(cccc4c2)Oc2ccccc2-3)cc1 ; c1ccc(-c2ccccc2N(c2ccc(-c3cccc4ccccc34)cc2)c2ccccc2-c2cc3c4c(cccc4c2)Oc2ccccc2-3)cc1 ; c1ccc(-c2ccccc2N(c2ccccc2-c2cc3c4c(cccc4c2)Oc2ccccc2-3)c2ccccc2-c2cccc3ccccc23)cc1]

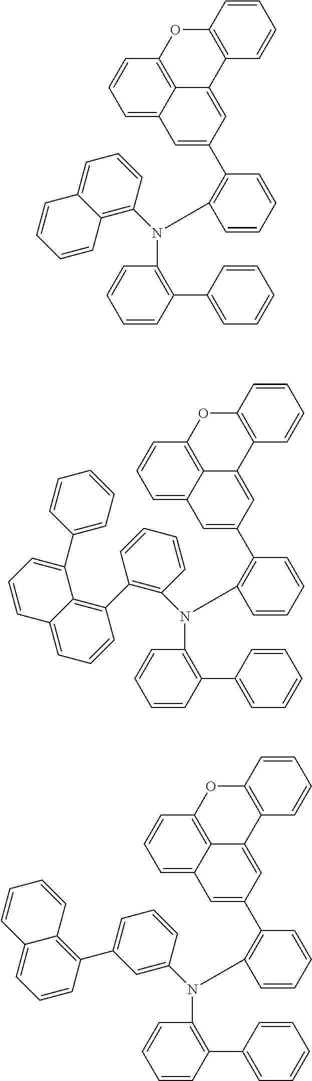 [SMILES: c1ccc(-c2ccccc2N(c2ccccc2-c2cc3c4c(cccc4c2)Oc2ccccc2-3)c2cccc3ccccc23)cc1 ; c1ccc(-c2ccccc2N(c2ccccc2-c2cc3c4c(cccc4c2)Oc2ccccc2-3)c2ccccc2-c2cccc3cccc(-c4ccccc4)c23)cc1 ; c1ccc(-c2ccccc2N(c2cccc(-c3cccc4ccccc34)c2)c2ccccc2-c2cc3c4c(cccc4c2)Oc2ccccc2-3)cc1]

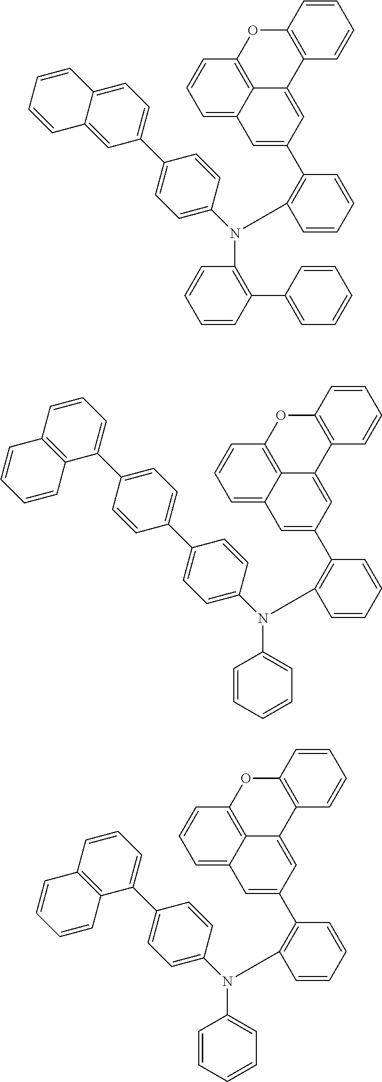

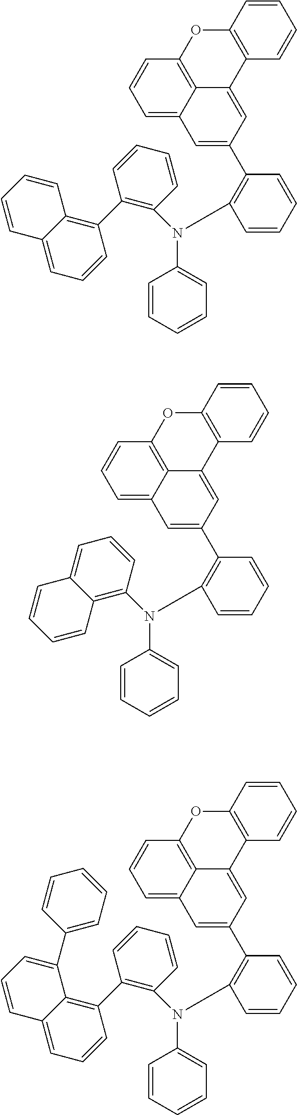 [SMILES: c1ccc(N(c2ccccc2-c2cc3c4c(cccc4c2)Oc2ccccc2-3)c2ccccc2-c2cccc3ccccc23)cc1 ; c1ccc(N(c2ccccc2-c2cc3c4c(cccc4c2)Oc2ccccc2-3)c2cccc3ccccc23)cc1 ; c1ccc(-c2cccc3cccc(-c4ccccc4N(c4ccccc4)c4ccccc4-c4cc5c6c(cccc6c4)Oc4ccccc4-5)c23)cc1]

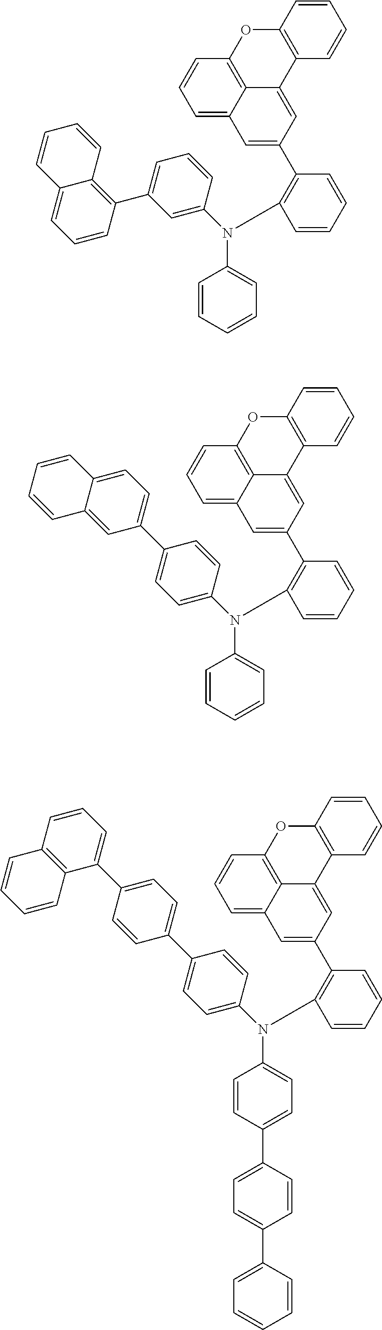 [SMILES: c1ccc(N(c2cccc(-c3cccc4ccccc34)c2)c2ccccc2-c2cc3c4c(cccc4c2)Oc2ccccc2-3)cc1 ; c1ccc(N(c2ccc(-c3ccc4ccccc4c3)cc2)c2ccccc2-c2cc3c4c(cccc4c2)Oc2ccccc2-3)cc1 ; c1ccc(-c2ccc(-c3ccc(N(c4ccc(-c5ccc(-c6cccc7ccccc67)cc5)cc4)c4ccccc4-c4cc5c6c(cccc6c4)Oc4ccccc4-5)cc3)cc2)cc1]

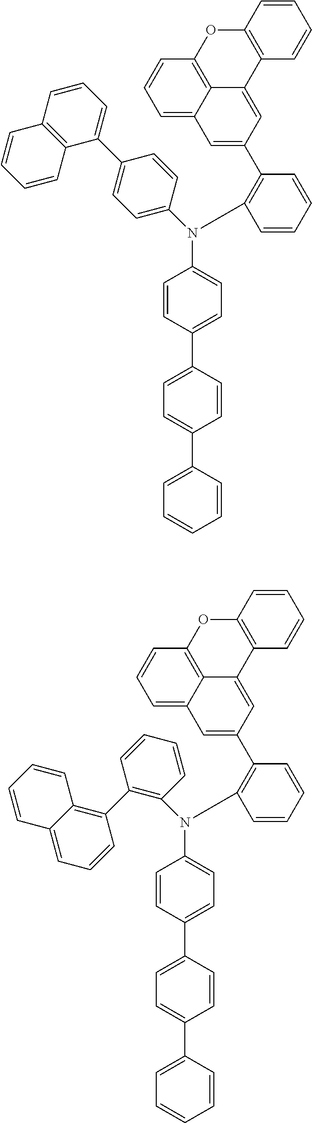 [SMILES: c1ccc(-c2ccc(-c3ccc(N(c4ccc(-c5cccc6ccccc56)cc4)c4ccccc4-c4cc5c6c(cccc6c4)Oc4ccccc4-5)cc3)cc2)cc1 ; c1ccc(-c2ccc(-c3ccc(N(c4ccccc4-c4cc5c6c(cccc6c4)Oc4ccccc4-5)c4ccccc4-c4cccc5ccccc45)cc3)cc2)cc1]

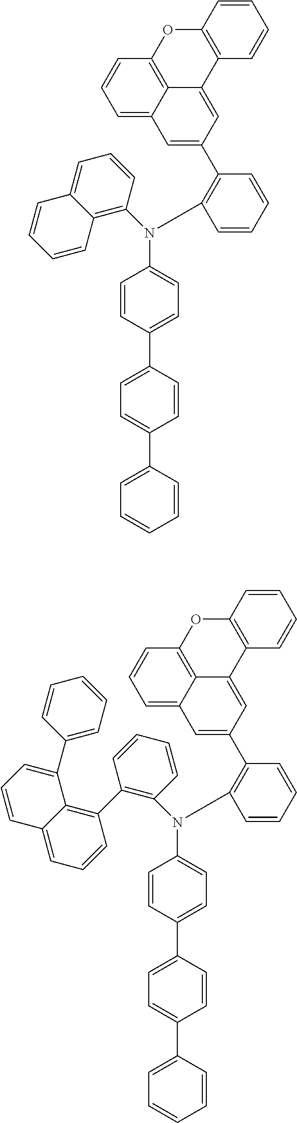 [SMILES: c1ccc(-c2ccc(-c3ccc(N(c4ccccc4-c4cc5c6c(cccc6c4)Oc4ccccc4-5)c4cccc5ccccc45)cc3)cc2)cc1 ; c1ccc(-c2ccc(-c3ccc(N(c4ccccc4-c4cc5c6c(cccc6c4)Oc4ccccc4-5)c4ccccc4-c4cccc5cccc(-c6ccccc6)c45)cc3)cc2)cc1]

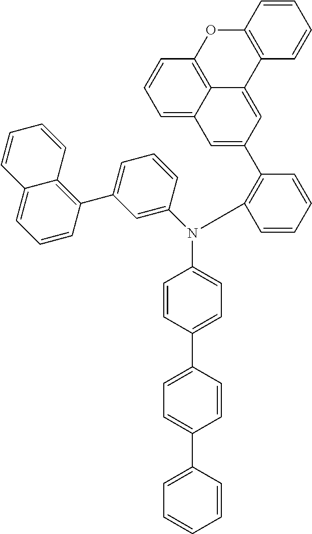 [SMILES: c1ccc(-c2ccc(-c3ccc(N(c4cccc(-c5cccc6ccccc56)c4)c4ccccc4-c4cc5c6c(cccc6c4)Oc4ccccc4-5)cc3)cc2)cc1]

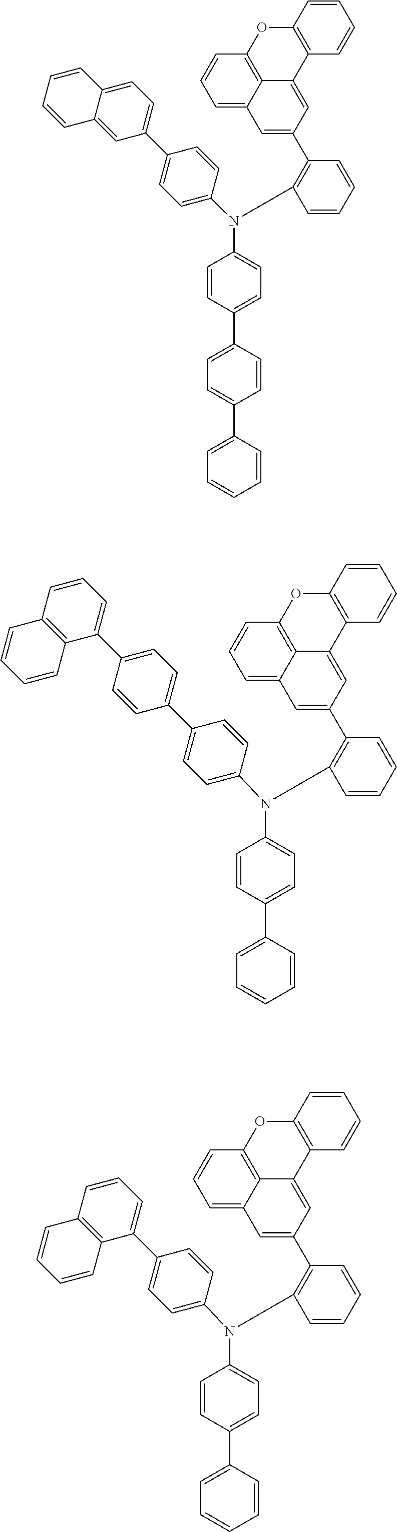 [SMILES: c1ccc(-c2ccc(-c3ccc(N(c4ccc(-c5ccc6ccccc6c5)cc4)c4ccccc4-c4cc5c6c(cccc6c4)Oc4ccccc4-5)cc3)cc2)cc1 ; c1ccc(-c2ccc(N(c3ccc(-c4ccc(-c5cccc6ccccc56)cc4)cc3)c3ccccc3-c3cc4c5c(cccc5c3)Oc3ccccc3-4)cc2)cc1 ; c1ccc(-c2ccc(N(c3ccc(-c4cccc5ccccc45)cc3)c3ccccc3-c3cc4c5c(cccc5c3)Oc3ccccc3-4)cc2)cc1]

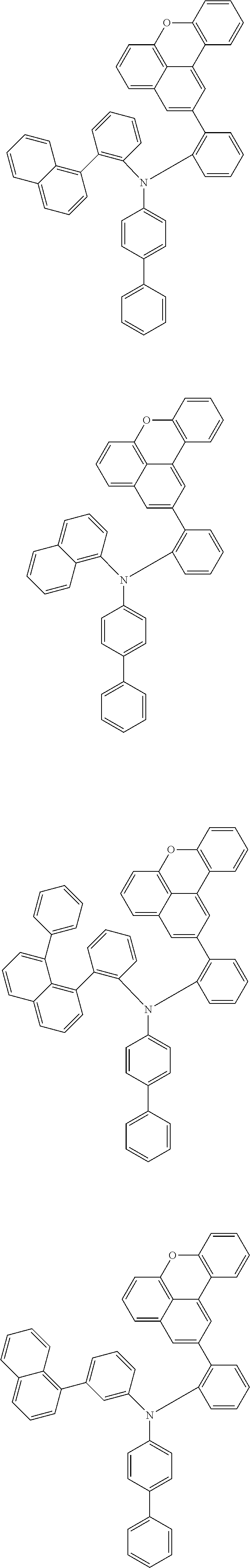 [SMILES: c1ccc(-c2ccc(N(c3ccccc3-c3cc4c5c(cccc5c3)Oc3ccccc3-4)c3ccccc3-c3cccc4ccccc34)cc2)cc1 ; c1ccc(-c2ccc(N(c3ccccc3-c3cc4c5c(cccc5c3)Oc3ccccc3-4)c3cccc4ccccc34)cc2)cc1 ; c1ccc(-c2ccc(N(c3ccccc3-c3cc4c5c(cccc5c3)Oc3ccccc3-4)c3ccccc3-c3cccc4cccc(-c5ccccc5)c34)cc2)cc1 ; c1ccc(-c2ccc(N(c3cccc(-c4cccc5ccccc45)c3)c3ccccc3-c3cc4c5c(cccc5c3)Oc3ccccc3-4)cc2)cc1]

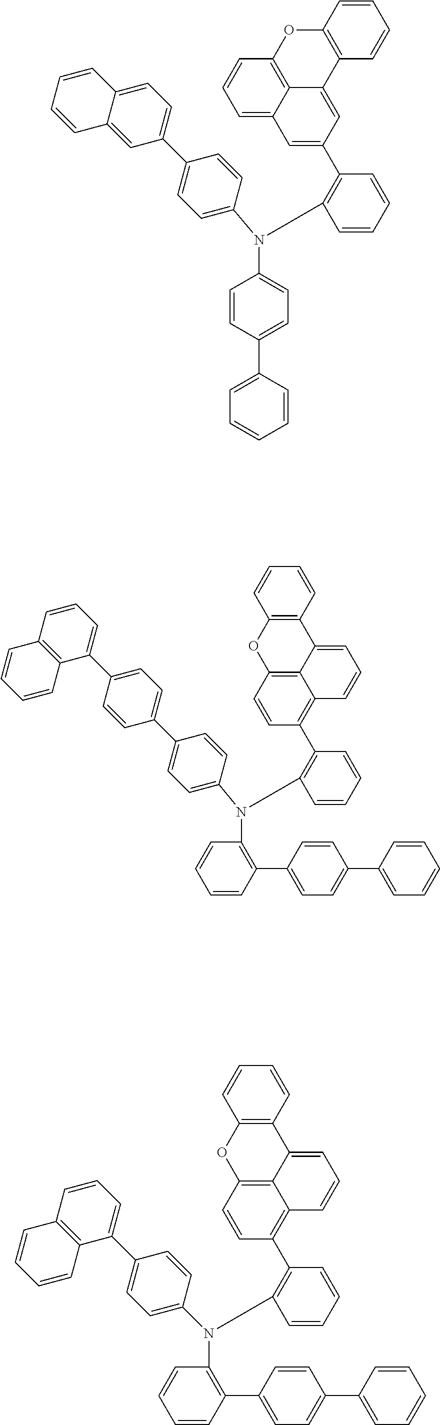 [SMILES: c1ccc(-c2ccc(N(c3ccc(-c4ccc5ccccc5c4)cc3)c3ccccc3-c3cc4c5c(cccc5c3)Oc3ccccc3-4)cc2)cc1 ; c1ccc(-c2ccc(-c3ccccc3N(c3ccc(-c4ccc(-c5cccc6ccccc56)cc4)cc3)c3ccccc3-c3ccc4c5c(cccc35)-c3ccccc3O4)cc2)cc1 ; c1ccc(-c2ccc(-c3ccccc3N(c3ccc(-c4cccc5ccccc45)cc3)c3ccccc3-c3ccc4c5c(cccc35)-c3ccccc3O4)cc2)cc1]

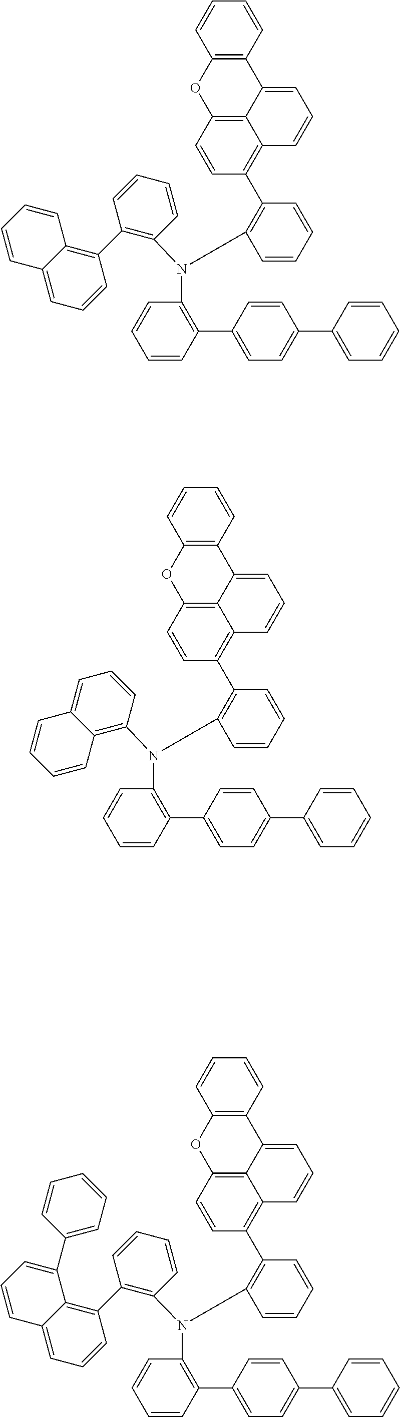 [SMILES: c1ccc(-c2ccc(-c3ccccc3N(c3ccccc3-c3cccc4ccccc34)c3ccccc3-c3ccc4c5c(cccc35)-c3ccccc3O4)cc2)cc1 ; c1ccc(-c2ccc(-c3ccccc3N(c3ccccc3-c3ccc4c5c(cccc35)-c3ccccc3O4)c3cccc4ccccc34)cc2)cc1 ; c1ccc(-c2ccc(-c3ccccc3N(c3ccccc3-c3ccc4c5c(cccc35)-c3ccccc3O4)c3ccccc3-c3cccc4cccc(-c5ccccc5)c34)cc2)cc1]

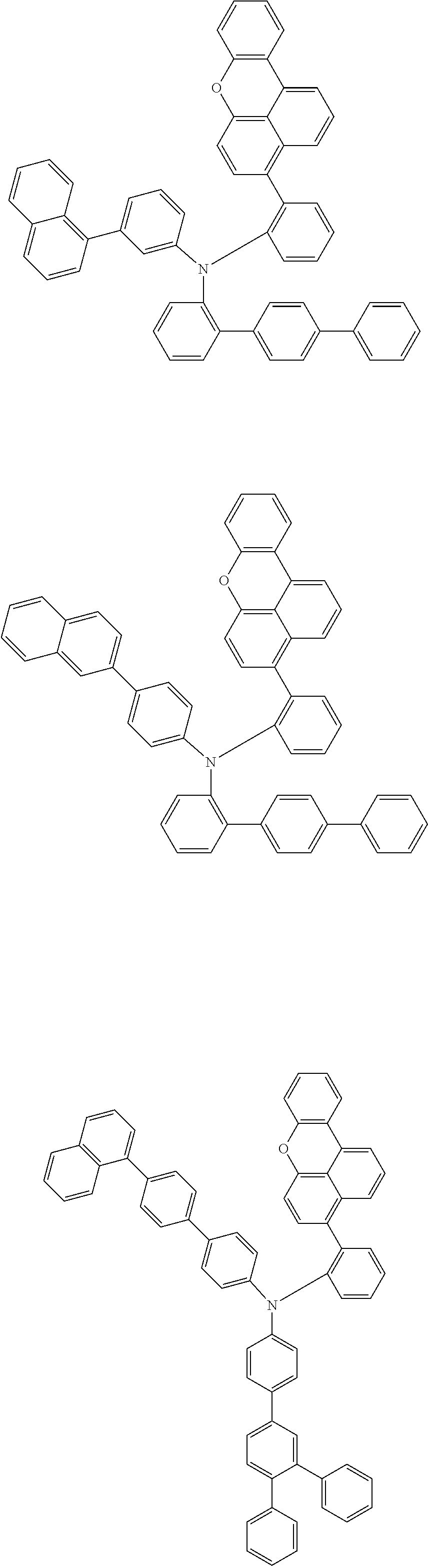 [SMILES: c1ccc(-c2ccc(-c3ccccc3N(c3cccc(-c4cccc5ccccc45)c3)c3ccccc3-c3ccc4c5c(cccc35)-c3ccccc3O4)cc2)cc1 ; c1ccc(-c2ccc(-c3ccccc3N(c3ccc(-c4ccc5ccccc5c4)cc3)c3ccccc3-c3ccc4c5c(cccc35)-c3ccccc3O4)cc2)cc1 ; c1ccc(-c2ccc(-c3ccc(N(c4ccc(-c5ccc(-c6cccc7ccccc67)cc5)cc4)c4ccccc4-c4ccc5c6c(cccc46)-c4ccccc4O5)cc3)cc2-c2ccccc2)cc1]

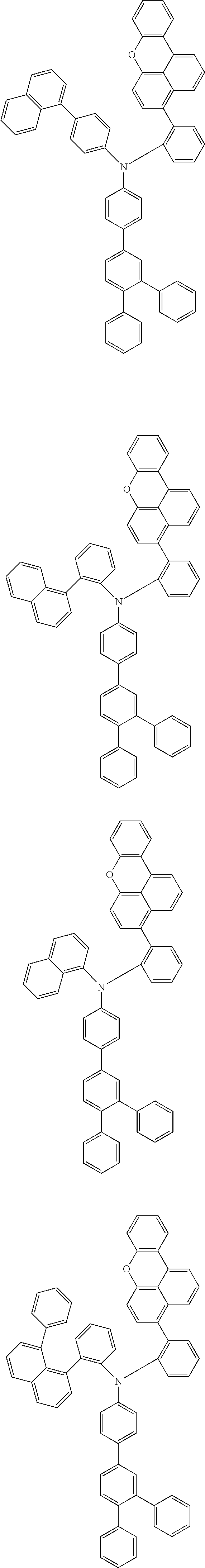 [SMILES: c1ccc(-c2ccc(-c3ccc(N(c4ccc(-c5cccc6ccccc56)cc4)c4ccccc4-c4ccc5c6c(cccc46)-c4ccccc4O5)cc3)cc2-c2ccccc2)cc1 ; c1ccc(-c2ccc(-c3ccc(N(c4ccccc4-c4cccc5ccccc45)c4ccccc4-c4ccc5c6c(cccc46)-c4ccccc4O5)cc3)cc2-c2ccccc2)cc1 ; c1ccc(-c2ccc(-c3ccc(N(c4ccccc4-c4ccc5c6c(cccc46)-c4ccccc4O5)c4cccc5ccccc45)cc3)cc2-c2ccccc2)cc1 ; c1ccc(-c2ccc(-c3ccc(N(c4ccccc4-c4ccc5c6c(cccc46)-c4ccccc4O5)c4ccccc4-c4cccc5cccc(-c6ccccc6)c45)cc3)cc2-c2ccccc2)cc1]

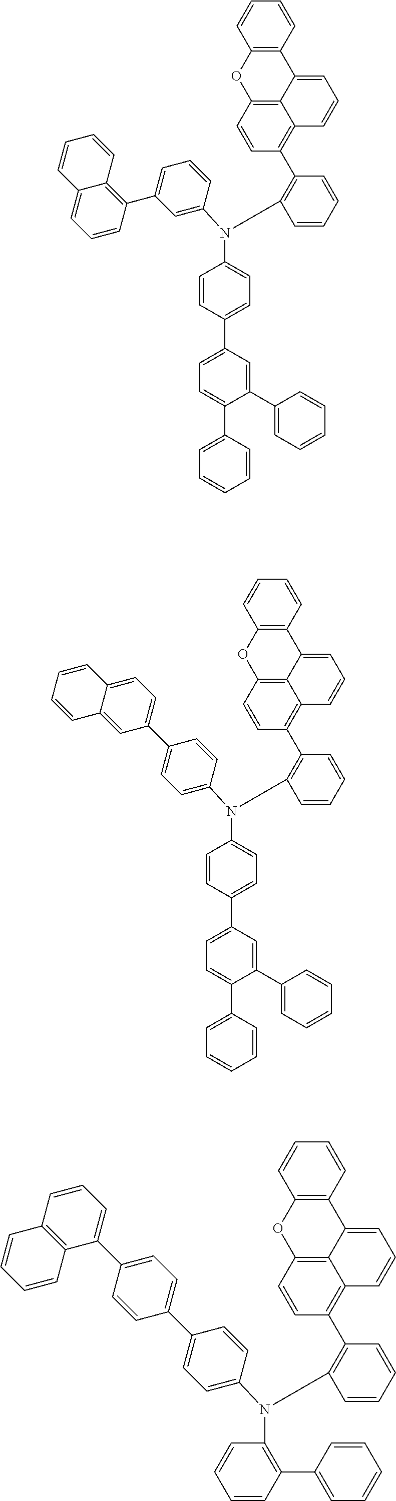 [SMILES: c1ccc(-c2ccc(-c3ccc(N(c4cccc(-c5cccc6ccccc56)c4)c4ccccc4-c4ccc5c6c(cccc46)-c4ccccc4O5)cc3)cc2-c2ccccc2)cc1 ; c1ccc(-c2ccc(-c3ccc(N(c4ccc(-c5ccc6ccccc6c5)cc4)c4ccccc4-c4ccc5c6c(cccc46)-c4ccccc4O5)cc3)cc2-c2ccccc2)cc1 ; c1ccc(-c2ccccc2N(c2ccc(-c3ccc(-c4cccc5ccccc45)cc3)cc2)c2ccccc2-c2ccc3c4c(cccc24)-c2ccccc2O3)cc1]

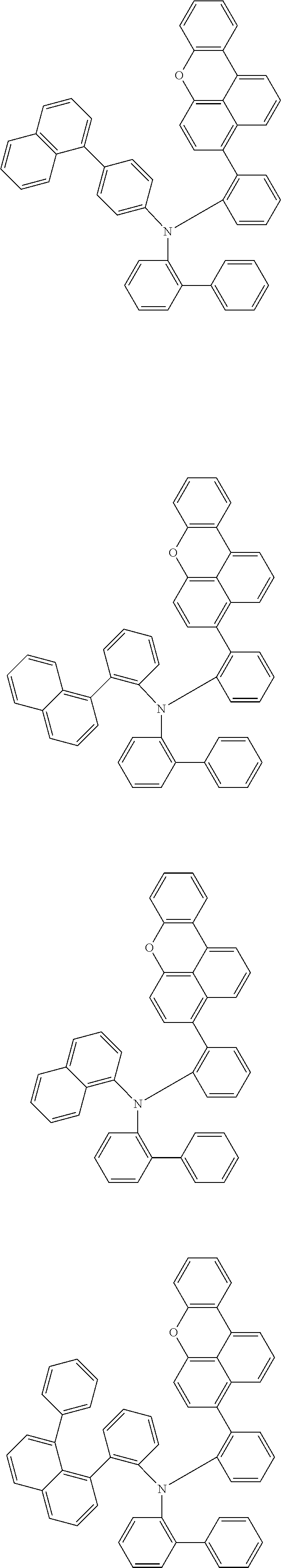 [SMILES: c1ccc(-c2ccccc2N(c2ccc(-c3cccc4ccccc34)cc2)c2ccccc2-c2ccc3c4c(cccc24)-c2ccccc2O3)cc1 ; c1ccc(-c2ccccc2N(c2ccccc2-c2cccc3ccccc23)c2ccccc2-c2ccc3c4c(cccc24)-c2ccccc2O3)cc1 ; c1ccc(-c2ccccc2N(c2ccccc2-c2ccc3c4c(cccc24)-c2ccccc2O3)c2cccc3ccccc23)cc1 ; c1ccc(-c2ccccc2N(c2ccccc2-c2ccc3c4c(cccc24)-c2ccccc2O3)c2ccccc2-c2cccc3cccc(-c4ccccc4)c23)cc1]

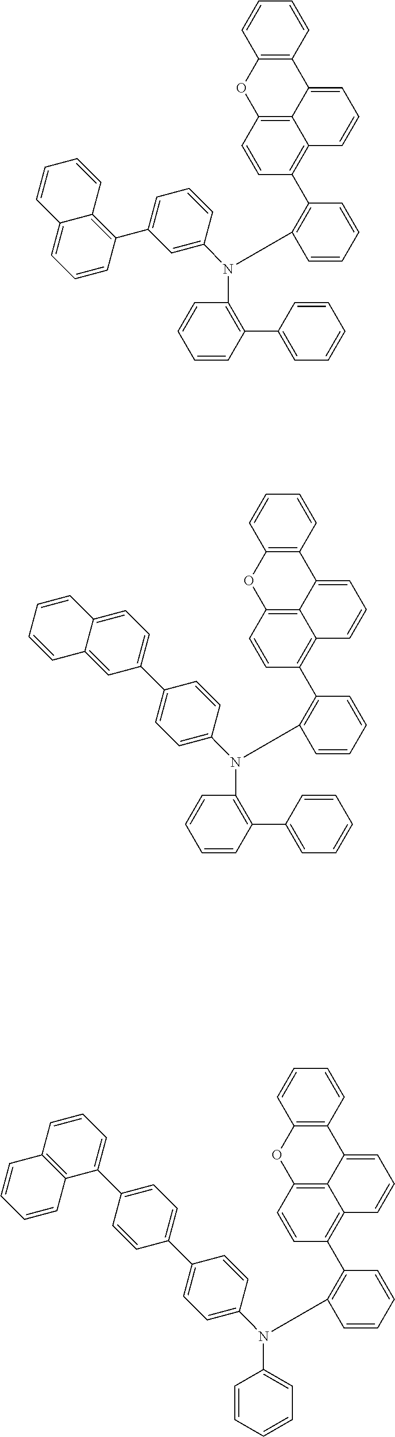 [SMILES: c1ccc(-c2ccccc2N(c2cccc(-c3cccc4ccccc34)c2)c2ccccc2-c2ccc3c4c(cccc24)-c2ccccc2O3)cc1 ; c1ccc(-c2ccccc2N(c2ccc(-c3ccc4ccccc4c3)cc2)c2ccccc2-c2ccc3c4c(cccc24)-c2ccccc2O3)cc1 ; c1ccc(N(c2ccc(-c3ccc(-c4cccc5ccccc45)cc3)cc2)c2ccccc2-c2ccc3c4c(cccc24)-c2ccccc2O3)cc1]

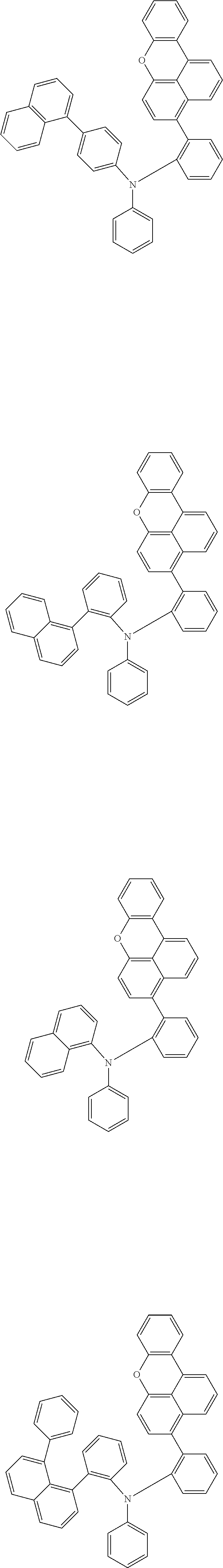 [SMILES: c1ccc(N(c2ccc(-c3cccc4ccccc34)cc2)c2ccccc2-c2ccc3c4c(cccc24)-c2ccccc2O3)cc1 ; c1ccc(N(c2ccccc2-c2cccc3ccccc23)c2ccccc2-c2ccc3c4c(cccc24)-c2ccccc2O3)cc1 ; c1ccc(N(c2ccccc2-c2ccc3c4c(cccc24)-c2ccccc2O3)c2cccc3ccccc23)cc1 ; c1ccc(-c2cccc3cccc(-c4ccccc4N(c4ccccc4)c4ccccc4-c4ccc5c6c(cccc46)-c4ccccc4O5)c23)cc1]

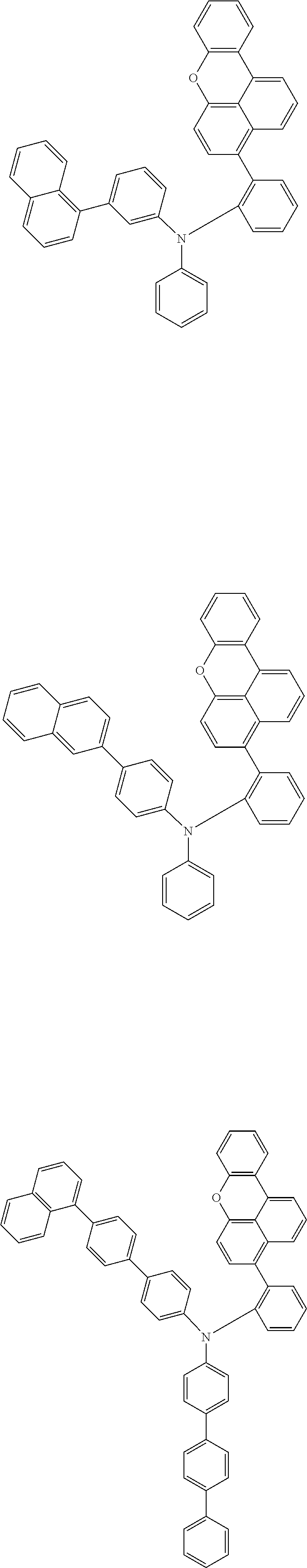 [SMILES: c1ccc(N(c2cccc(-c3cccc4ccccc34)c2)c2ccccc2-c2ccc3c4c(cccc24)-c2ccccc2O3)cc1 ; c1ccc(N(c2ccc(-c3ccc4ccccc4c3)cc2)c2ccccc2-c2ccc3c4c(cccc24)-c2ccccc2O3)cc1 ; c1ccc(-c2ccc(-c3ccc(N(c4ccc(-c5ccc(-c6cccc7ccccc67)cc5)cc4)c4ccccc4-c4ccc5c6c(cccc46)-c4ccccc4O5)cc3)cc2)cc1]

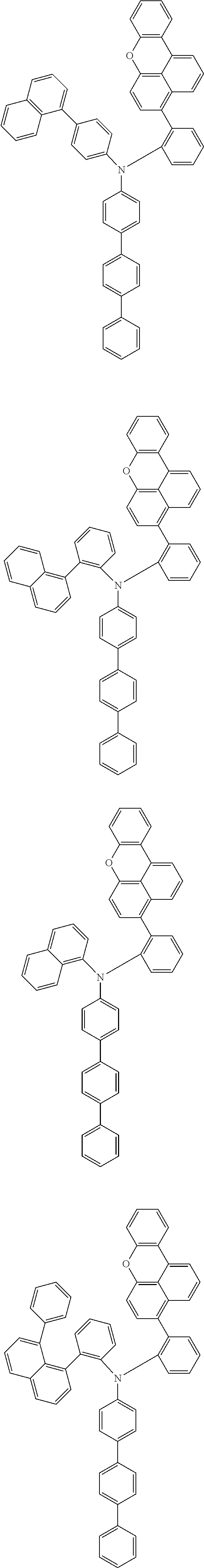 [SMILES: c1ccc(-c2ccc(-c3ccc(N(c4ccc(-c5cccc6ccccc56)cc4)c4ccccc4-c4ccc5c6c(cccc46)-c4ccccc4O5)cc3)cc2)cc1 ; c1ccc(-c2ccc(-c3ccc(N(c4ccccc4-c4cccc5ccccc45)c4ccccc4-c4ccc5c6c(cccc46)-c4ccccc4O5)cc3)cc2)cc1 ; c1ccc(-c2ccc(-c3ccc(N(c4ccccc4-c4ccc5c6c(cccc46)-c4ccccc4O5)c4cccc5ccccc45)cc3)cc2)cc1 ; c1ccc(-c2ccc(-c3ccc(N(c4ccccc4-c4ccc5c6c(cccc46)-c4ccccc4O5)c4ccccc4-c4cccc5cccc(-c6ccccc6)c45)cc3)cc2)cc1]

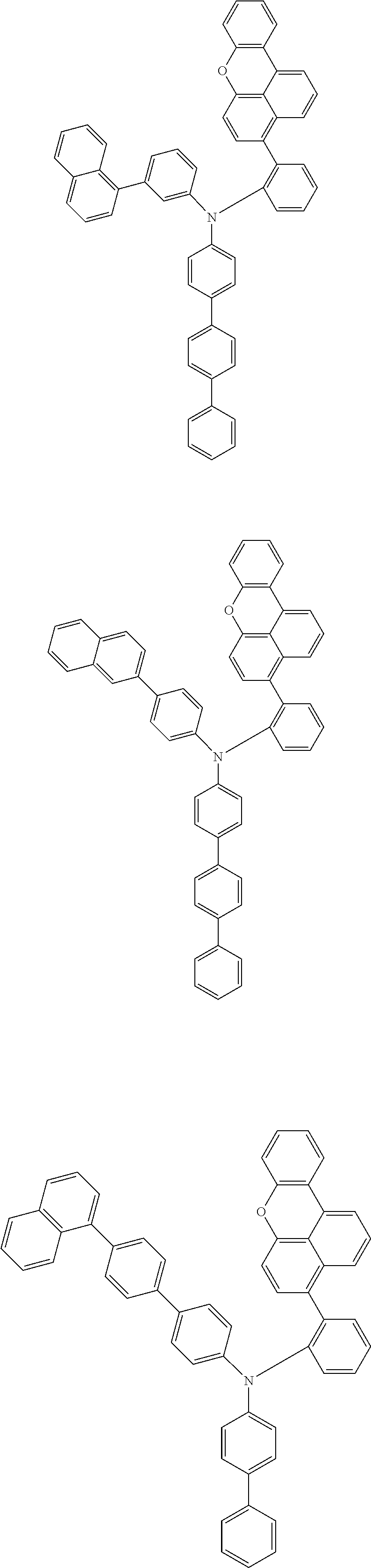 [SMILES: c1ccc(-c2ccc(-c3ccc(N(c4cccc(-c5cccc6ccccc56)c4)c4ccccc4-c4ccc5c6c(cccc46)-c4ccccc4O5)cc3)cc2)cc1 ; c1ccc(-c2ccc(-c3ccc(N(c4ccc(-c5ccc6ccccc6c5)cc4)c4ccccc4-c4ccc5c6c(cccc46)-c4ccccc4O5)cc3)cc2)cc1 ; c1ccc(-c2ccc(N(c3ccc(-c4ccc(-c5cccc6ccccc56)cc4)cc3)c3ccccc3-c3ccc4c5c(cccc35)-c3ccccc3O4)cc2)cc1]

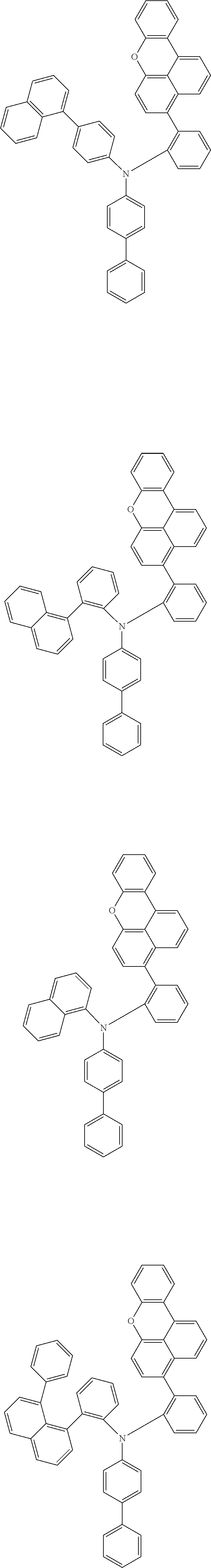 [SMILES: c1ccc(-c2ccc(N(c3ccc(-c4cccc5ccccc45)cc3)c3ccccc3-c3ccc4c5c(cccc35)-c3ccccc3O4)cc2)cc1 ; c1ccc(-c2ccc(N(c3ccccc3-c3cccc4ccccc34)c3ccccc3-c3ccc4c5c(cccc35)-c3ccccc3O4)cc2)cc1 ; c1ccc(-c2ccc(N(c3ccccc3-c3ccc4c5c(cccc35)-c3ccccc3O4)c3cccc4ccccc34)cc2)cc1 ; c1ccc(-c2ccc(N(c3ccccc3-c3ccc4c5c(cccc35)-c3ccccc3O4)c3ccccc3-c3cccc4cccc(-c5ccccc5)c34)cc2)cc1]

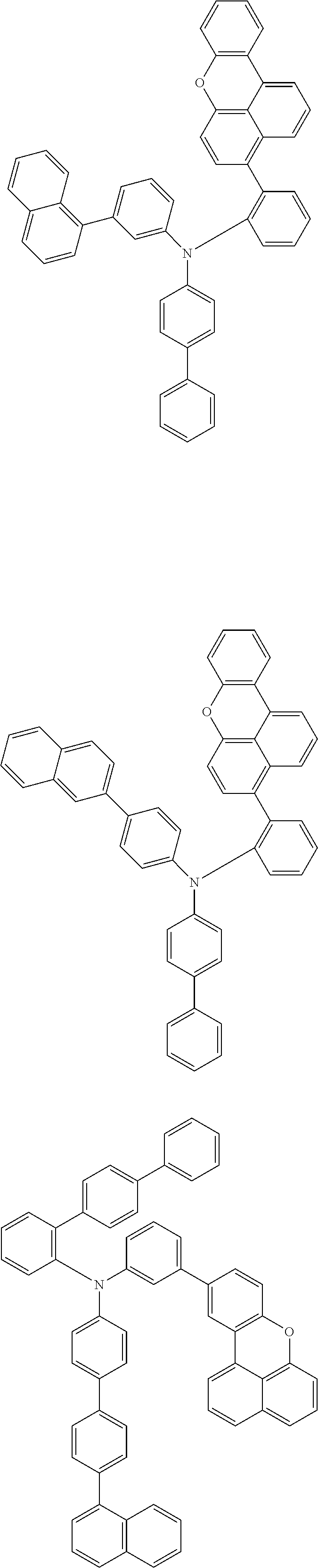 [SMILES: c1ccc(-c2ccc(N(c3cccc(-c4cccc5ccccc45)c3)c3ccccc3-c3ccc4c5c(cccc35)-c3ccccc3O4)cc2)cc1 ; c1ccc(-c2ccc(N(c3ccc(-c4ccc5ccccc5c4)cc3)c3ccccc3-c3ccc4c5c(cccc35)-c3ccccc3O4)cc2)cc1 ; c1ccc(-c2ccc(-c3ccccc3N(c3ccc(-c4ccc(-c5cccc6ccccc56)cc4)cc3)c3cccc(-c4ccc5c(c4)-c4cccc6cccc(c46)O5)c3)cc2)cc1]

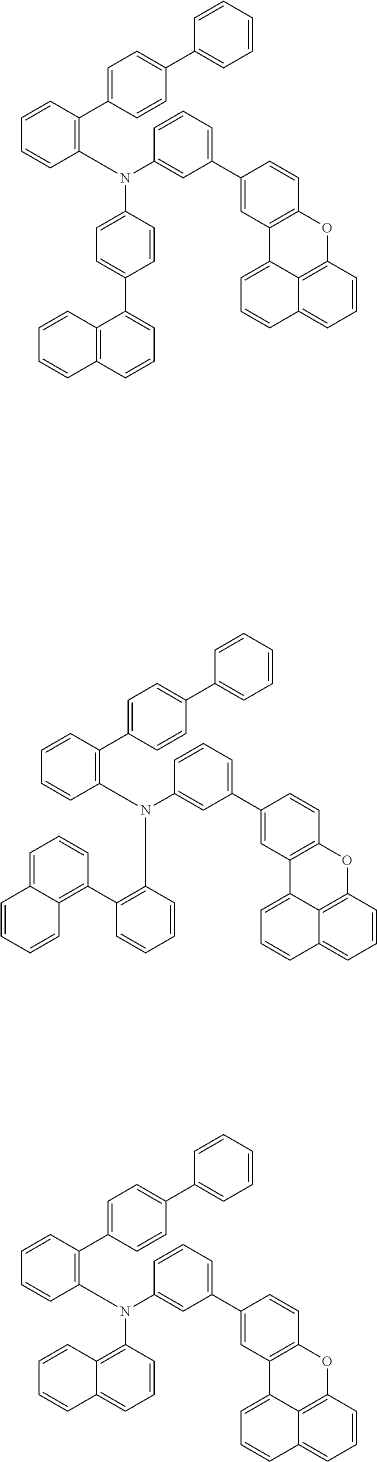 [SMILES: c1ccc(-c2ccc(-c3ccccc3N(c3ccc(-c4cccc5ccccc45)cc3)c3cccc(-c4ccc5c(c4)-c4cccc6cccc(c46)O5)c3)cc2)cc1 ; c1ccc(-c2ccc(-c3ccccc3N(c3cccc(-c4ccc5c(c4)-c4cccc6cccc(c46)O5)c3)c3ccccc3-c3cccc4ccccc34)cc2)cc1 ; c1ccc(-c2ccc(-c3ccccc3N(c3cccc(-c4ccc5c(c4)-c4cccc6cccc(c46)O5)c3)c3cccc4ccccc34)cc2)cc1]

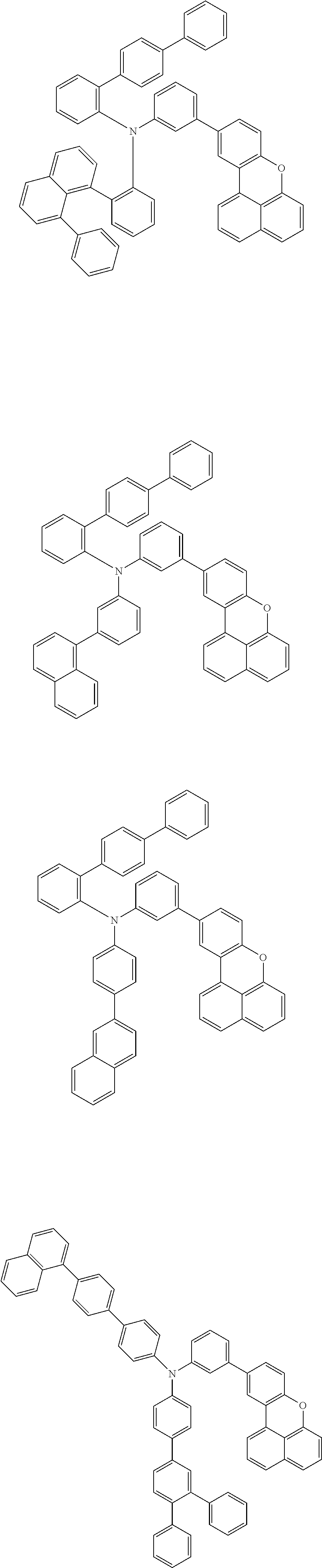 [SMILES: c1ccc(-c2ccc(-c3ccccc3N(c3cccc(-c4ccc5c(c4)-c4cccc6cccc(c46)O5)c3)c3ccccc3-c3cccc4cccc(-c5ccccc5)c34)cc2)cc1 ; c1ccc(-c2ccc(-c3ccccc3N(c3cccc(-c4ccc5c(c4)-c4cccc6cccc(c46)O5)c3)c3cccc(-c4cccc5ccccc45)c3)cc2)cc1 ; c1ccc(-c2ccc(-c3ccccc3N(c3ccc(-c4ccc5ccccc5c4)cc3)c3cccc(-c4ccc5c(c4)-c4cccc6cccc(c46)O5)c3)cc2)cc1 ; c1ccc(-c2ccc(-c3ccc(N(c4ccc(-c5ccc(-c6cccc7ccccc67)cc5)cc4)c4cccc(-c5ccc6c(c5)-c5cccc7cccc(c57)O6)c4)cc3)cc2-c2ccccc2)cc1]

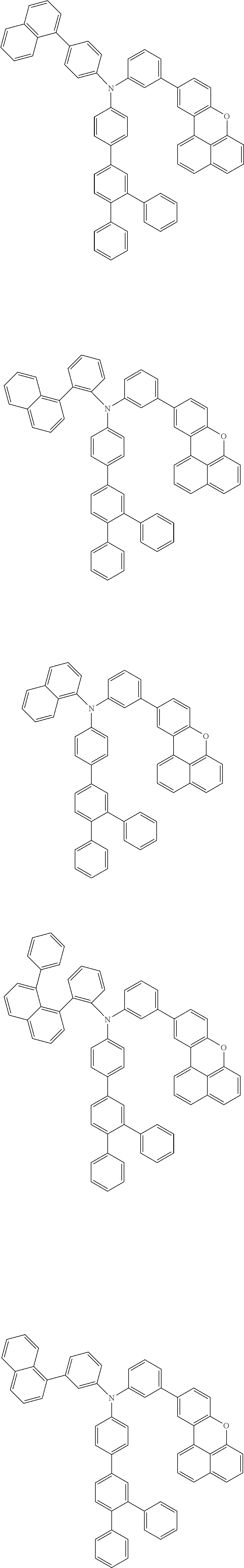

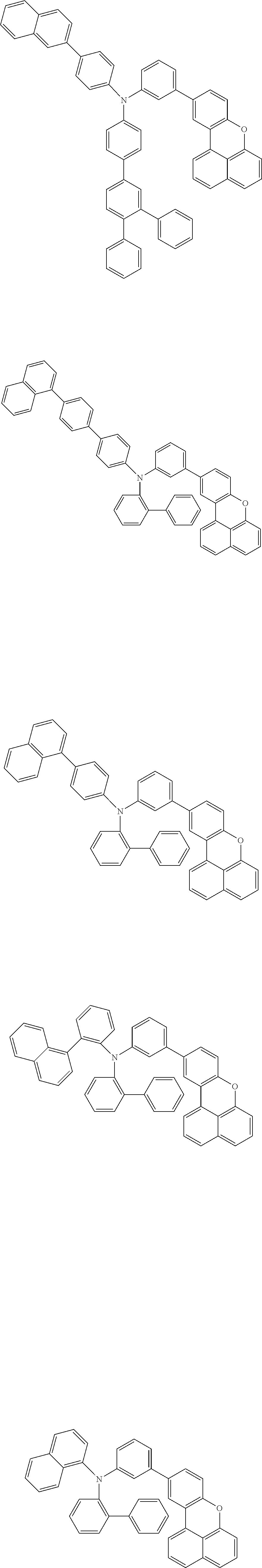 [SMILES: c1ccc(-c2ccc(-c3ccc(N(c4ccc(-c5ccc6ccccc6c5)cc4)c4cccc(-c5ccc6c(c5)-c5cccc7cccc(c57)O6)c4)cc3)cc2-c2ccccc2)cc1 ; c1ccc(-c2ccccc2N(c2ccc(-c3ccc(-c4cccc5ccccc45)cc3)cc2)c2cccc(-c3ccc4c(c3)-c3cccc5cccc(c35)O4)c2)cc1 ; c1ccc(-c2ccccc2N(c2ccc(-c3cccc4ccccc34)cc2)c2cccc(-c3ccc4c(c3)-c3cccc5cccc(c35)O4)c2)cc1 ; c1ccc(-c2ccccc2N(c2cccc(-c3ccc4c(c3)-c3cccc5cccc(c35)O4)c2)c2ccccc2-c2cccc3ccccc23)cc1 ; c1ccc(-c2ccccc2N(c2cccc(-c3ccc4c(c3)-c3cccc5cccc(c35)O4)c2)c2cccc3ccccc23)cc1]

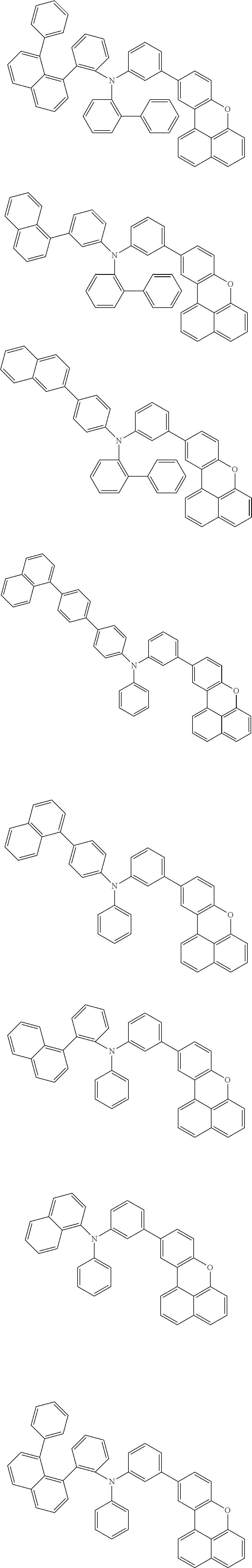

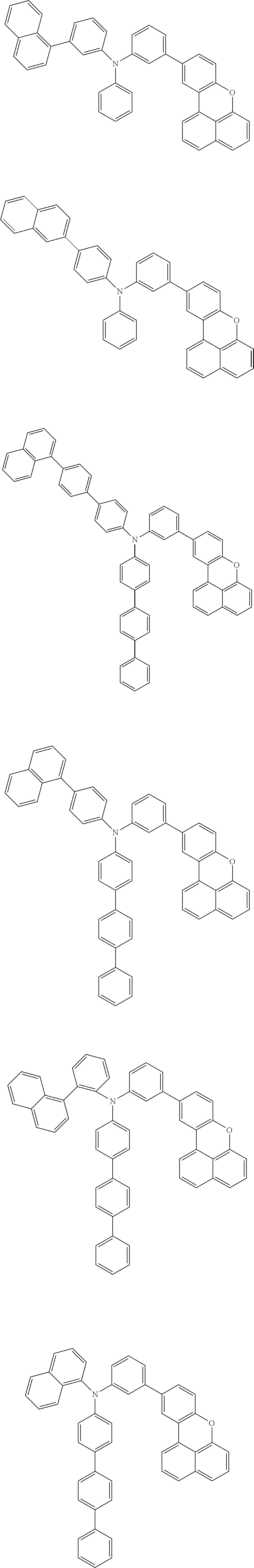

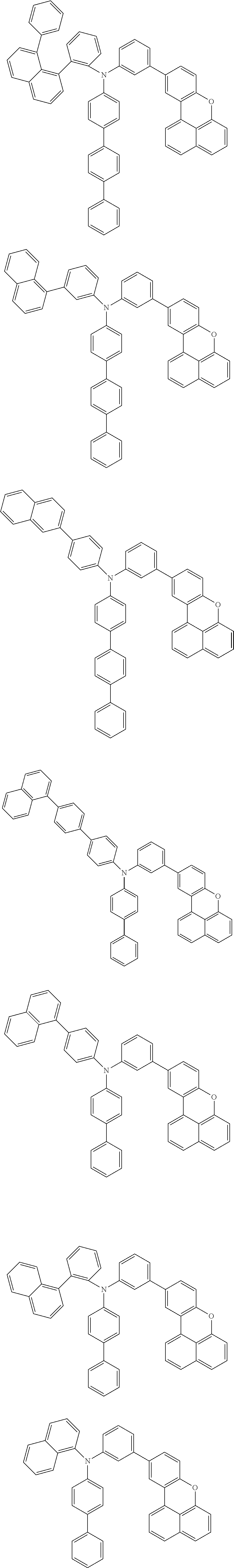

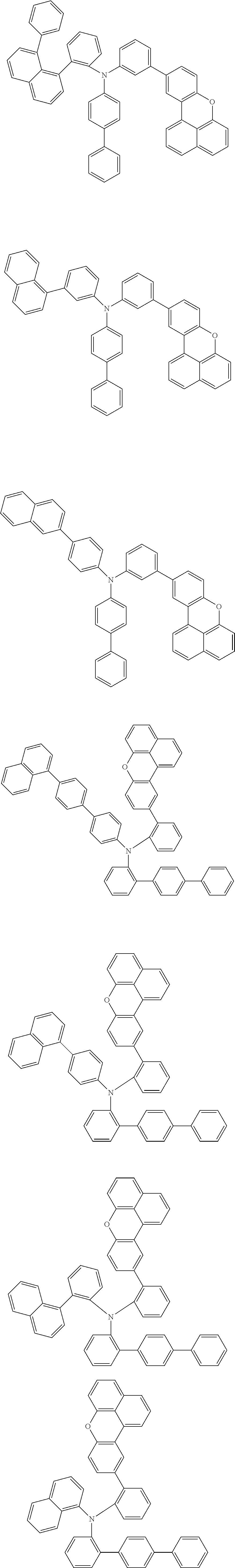

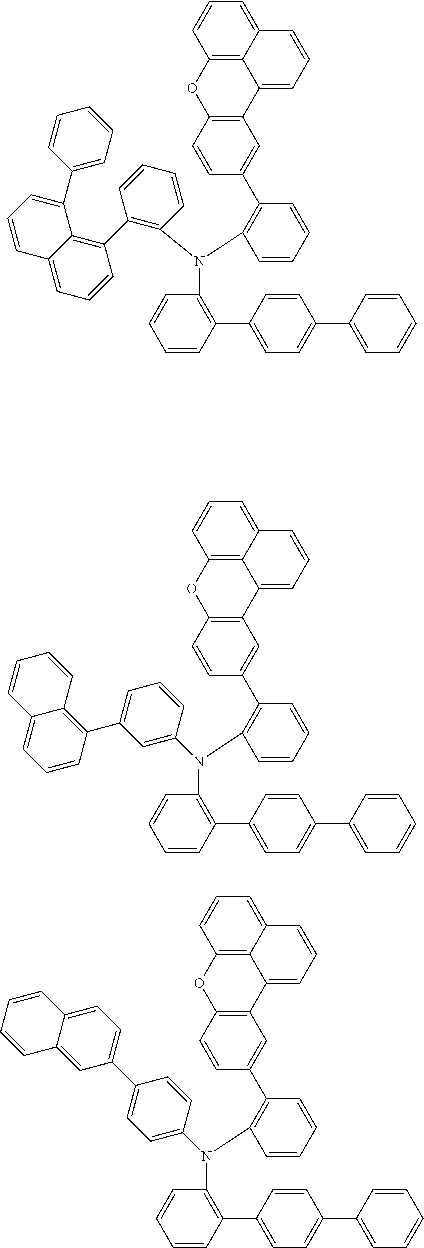

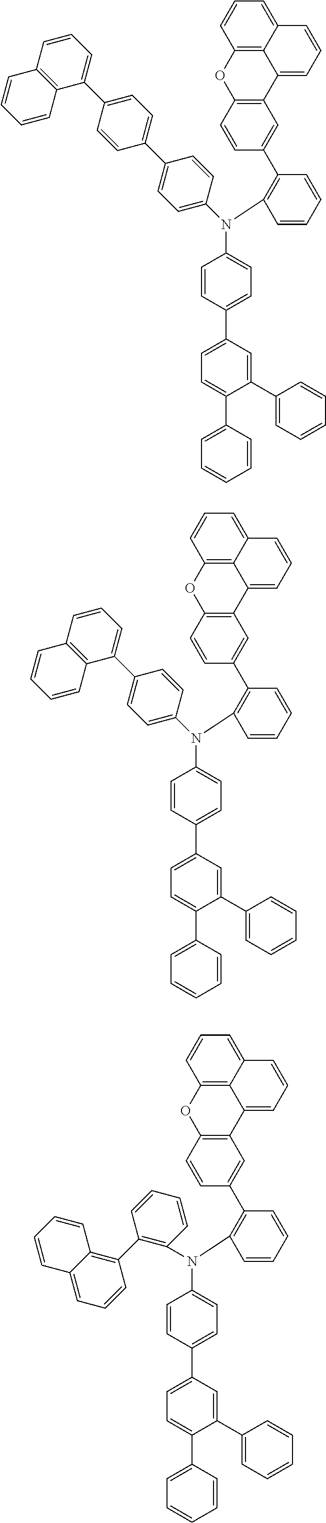

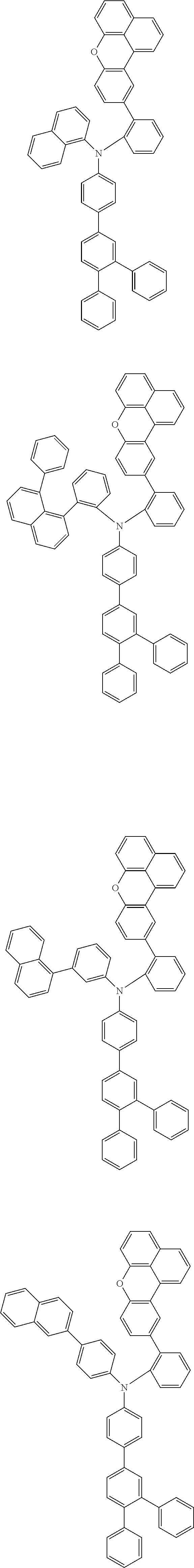 [SMILES: c1ccc(-c2ccc(-c3ccc(N(c4ccccc4-c4ccc5c(c4)-c4cccc6cccc(c46)O5)c4cccc5ccccc45)cc3)cc2-c2ccccc2)cc1 ; c1ccc(-c2ccc(-c3ccc(N(c4ccccc4-c4ccc5c(c4)-c4cccc6cccc(c46)O5)c4ccccc4-c4cccc5cccc(-c6ccccc6)c45)cc3)cc2-c2ccccc2)cc1 ; c1ccc(-c2ccc(-c3ccc(N(c4cccc(-c5cccc6ccccc56)c4)c4ccccc4-c4ccc5c(c4)-c4cccc6cccc(c46)O5)cc3)cc2-c2ccccc2)cc1 ; c1ccc(-c2ccc(-c3ccc(N(c4ccc(-c5ccc6ccccc6c5)cc4)c4ccccc4-c4ccc5c(c4)-c4cccc6cccc(c46)O5)cc3)cc2-c2ccccc2)cc1]

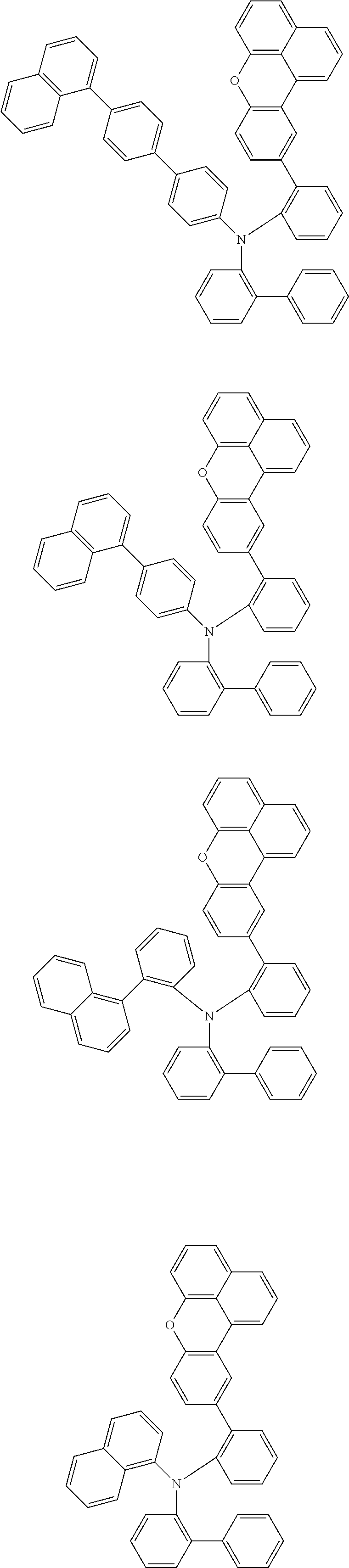 [SMILES: c1ccc(-c2ccccc2N(c2ccc(-c3ccc(-c4cccc5ccccc45)cc3)cc2)c2ccccc2-c2ccc3c(c2)-c2cccc4cccc(c24)O3)cc1 ; c1ccc(-c2ccccc2N(c2ccc(-c3cccc4ccccc34)cc2)c2ccccc2-c2ccc3c(c2)-c2cccc4cccc(c24)O3)cc1 ; c1ccc(-c2ccccc2N(c2ccccc2-c2ccc3c(c2)-c2cccc4cccc(c24)O3)c2ccccc2-c2cccc3ccccc23)cc1 ; c1ccc(-c2ccccc2N(c2ccccc2-c2ccc3c(c2)-c2cccc4cccc(c24)O3)c2cccc3ccccc23)cc1]

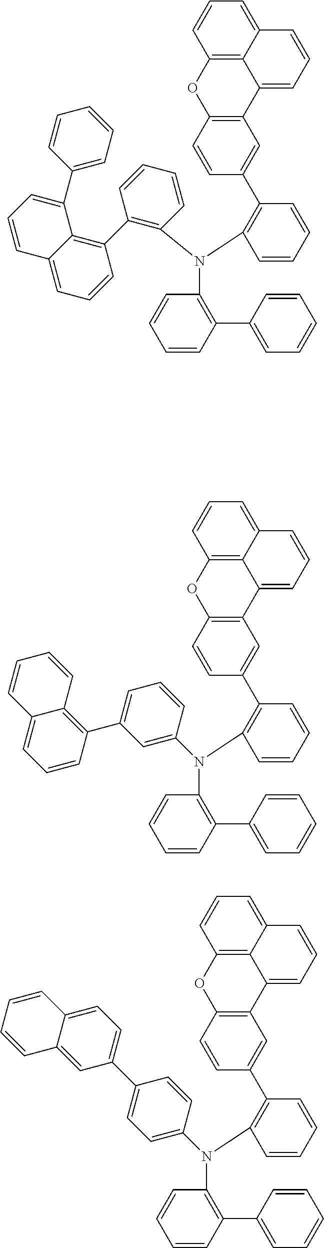

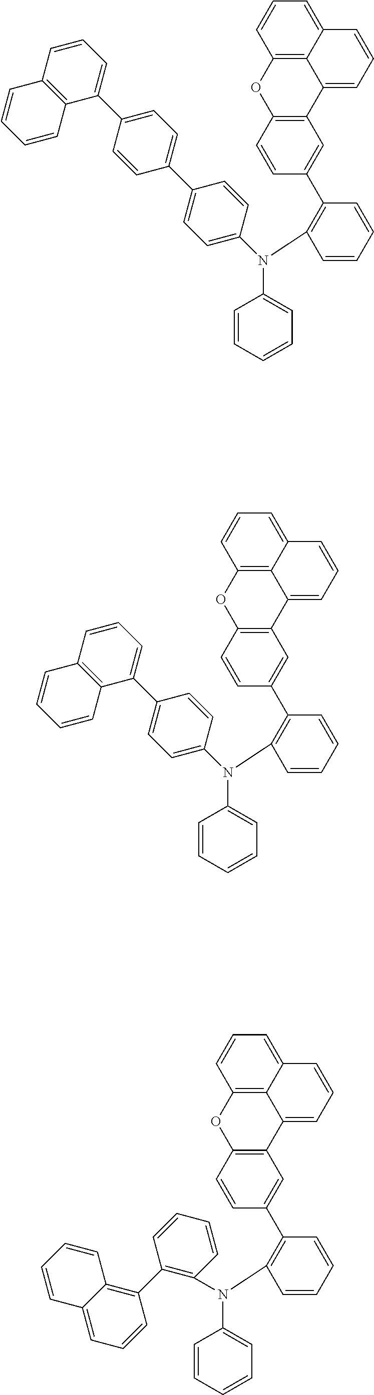 [SMILES: c1ccc(N(c2ccc(-c3ccc(-c4cccc5ccccc45)cc3)cc2)c2ccccc2-c2ccc3c(c2)-c2cccc4cccc(c24)O3)cc1 ; c1ccc(N(c2ccc(-c3cccc4ccccc34)cc2)c2ccccc2-c2ccc3c(c2)-c2cccc4cccc(c24)O3)cc1 ; c1ccc(N(c2ccccc2-c2ccc3c(c2)-c2cccc4cccc(c24)O3)c2ccccc2-c2cccc3ccccc23)cc1]

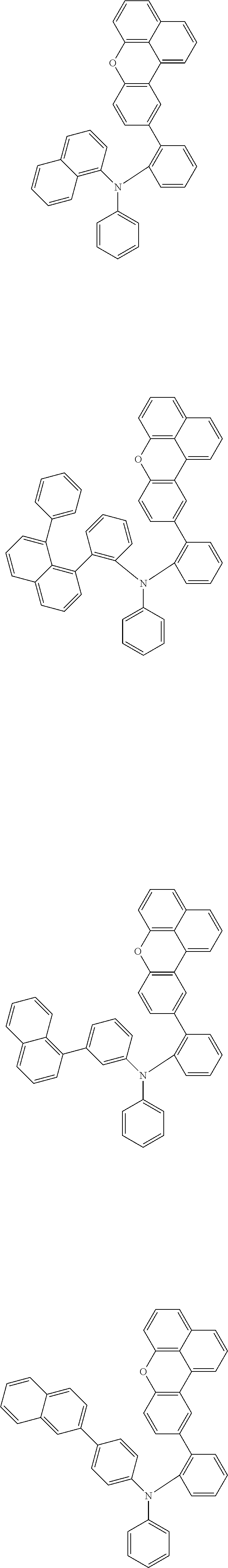 [SMILES: c1ccc(N(c2ccccc2-c2ccc3c(c2)-c2cccc4cccc(c24)O3)c2cccc3ccccc23)cc1 ; c1ccc(-c2cccc3cccc(-c4ccccc4N(c4ccccc4)c4ccccc4-c4ccc5c(c4)-c4cccc6cccc(c46)O5)c23)cc1 ; c1ccc(N(c2cccc(-c3cccc4ccccc34)c2)c2ccccc2-c2ccc3c(c2)-c2cccc4cccc(c24)O3)cc1 ; c1ccc(N(c2ccc(-c3ccc4ccccc4c3)cc2)c2ccccc2-c2ccc3c(c2)-c2cccc4cccc(c24)O3)cc1]

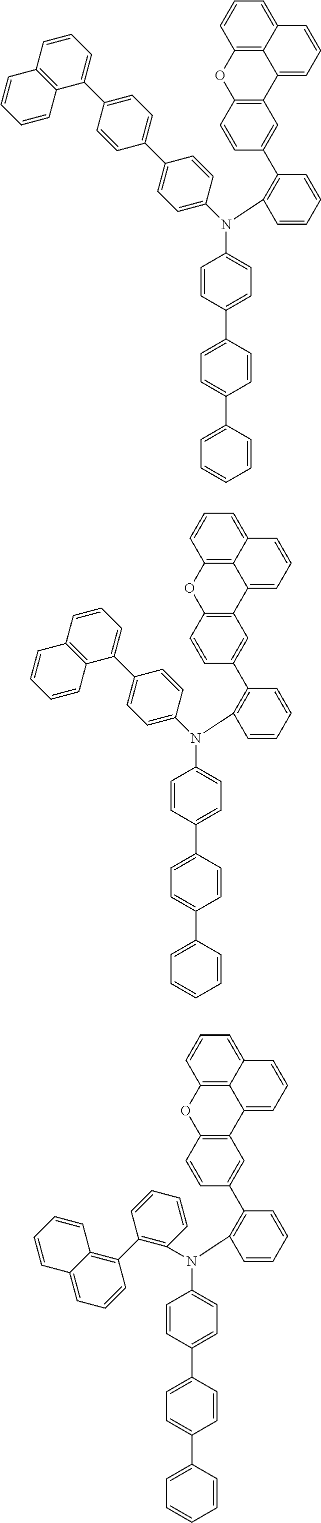

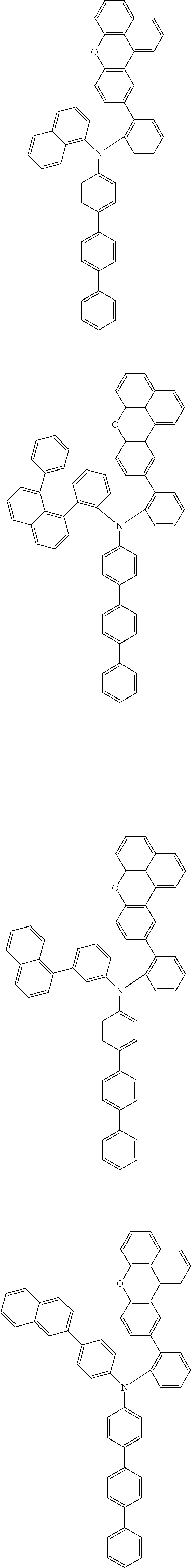 [SMILES: c1ccc(-c2ccc(-c3ccc(N(c4ccccc4-c4ccc5c(c4)-c4cccc6cccc(c46)O5)c4cccc5ccccc45)cc3)cc2)cc1 ; c1ccc(-c2ccc(-c3ccc(N(c4ccccc4-c4ccc5c(c4)-c4cccc6cccc(c46)O5)c4ccccc4-c4cccc5cccc(-c6ccccc6)c45)cc3)cc2)cc1 ; c1ccc(-c2ccc(-c3ccc(N(c4cccc(-c5cccc6ccccc56)c4)c4ccccc4-c4ccc5c(c4)-c4cccc6cccc(c46)O5)cc3)cc2)cc1 ; c1ccc(-c2ccc(-c3ccc(N(c4ccc(-c5ccc6ccccc6c5)cc4)c4ccccc4-c4ccc5c(c4)-c4cccc6cccc(c46)O5)cc3)cc2)cc1]

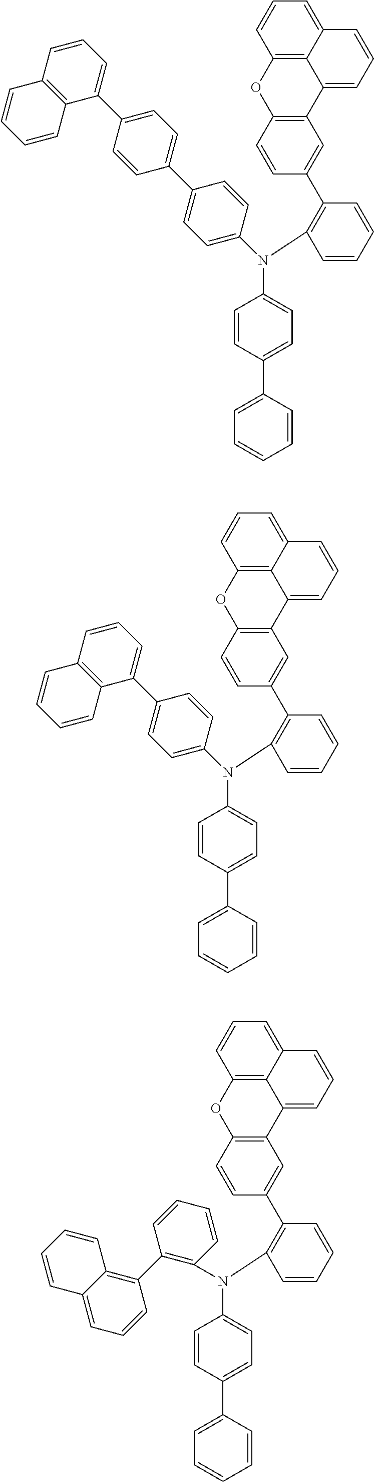 [SMILES: c1ccc(-c2ccc(N(c3ccc(-c4ccc(-c5cccc6ccccc56)cc4)cc3)c3ccccc3-c3ccc4c(c3)-c3cccc5cccc(c35)O4)cc2)cc1 ; c1ccc(-c2ccc(N(c3ccc(-c4cccc5ccccc45)cc3)c3ccccc3-c3ccc4c(c3)-c3cccc5cccc(c35)O4)cc2)cc1 ; c1ccc(-c2ccc(N(c3ccccc3-c3ccc4c(c3)-c3cccc5cccc(c35)O4)c3ccccc3-c3cccc4ccccc34)cc2)cc1]

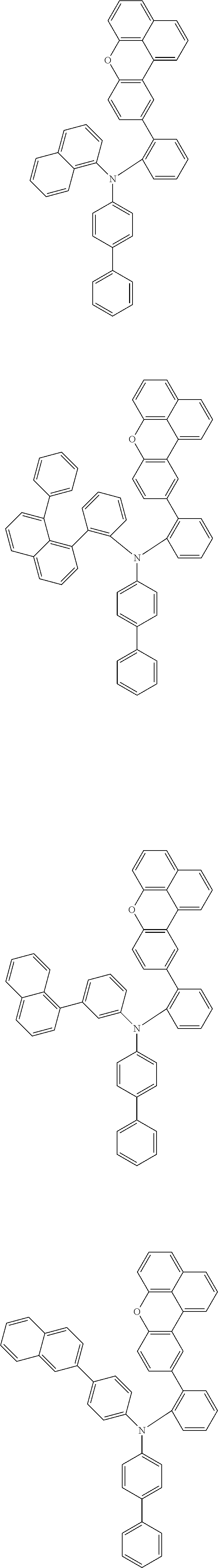 [SMILES: c1ccc(-c2ccc(N(c3ccccc3-c3ccc4c(c3)-c3cccc5cccc(c35)O4)c3cccc4ccccc34)cc2)cc1 ; c1ccc(-c2ccc(N(c3ccccc3-c3ccc4c(c3)-c3cccc5cccc(c35)O4)c3ccccc3-c3cccc4cccc(-c5ccccc5)c34)cc2)cc1 ; c1ccc(-c2ccc(N(c3cccc(-c4cccc5ccccc45)c3)c3ccccc3-c3ccc4c(c3)-c3cccc5cccc(c35)O4)cc2)cc1 ; c1ccc(-c2ccc(N(c3ccc(-c4ccc5ccccc5c4)cc3)c3ccccc3-c3ccc4c(c3)-c3cccc5cccc(c35)O4)cc2)cc1]

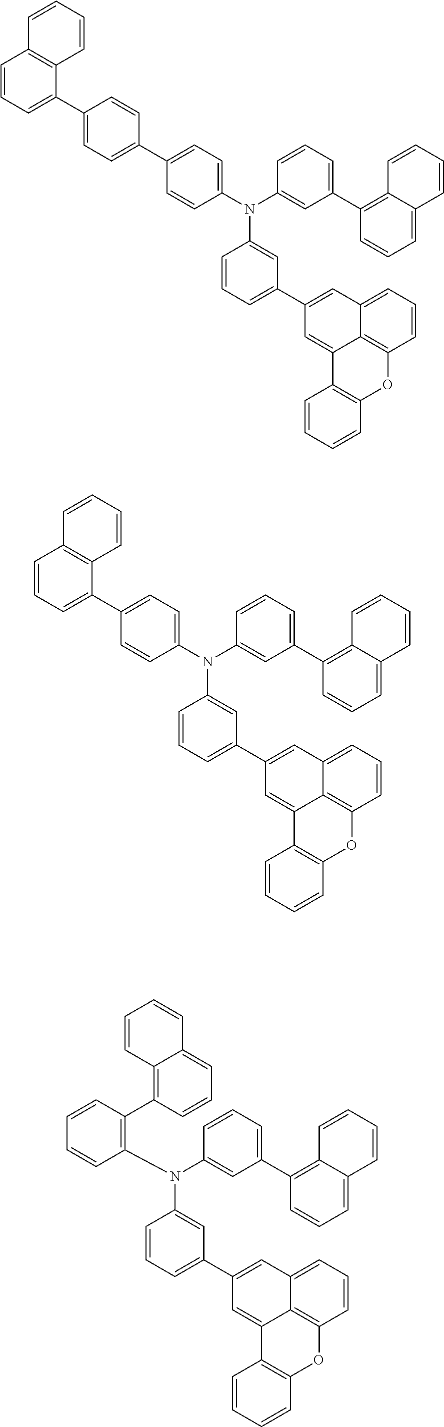 [SMILES: c1cc(-c2cc3c4c(cccc4c2)Oc2ccccc2-3)cc(N(c2ccc(-c3ccc(-c4cccc5ccccc45)cc3)cc2)c2cccc(-c3cccc4ccccc34)c2)c1 ; c1cc(-c2cc3c4c(cccc4c2)Oc2ccccc2-3)cc(N(c2ccc(-c3cccc4ccccc34)cc2)c2cccc(-c3cccc4ccccc34)c2)c1 ; c1cc(-c2cc3c4c(cccc4c2)Oc2ccccc2-3)cc(N(c2cccc(-c3cccc4ccccc34)c2)c2ccccc2-c2cccc3ccccc23)c1]

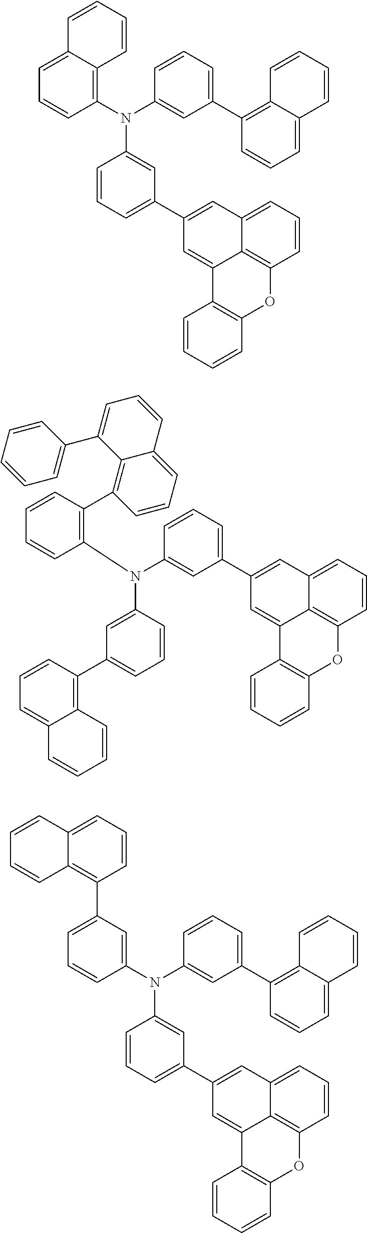

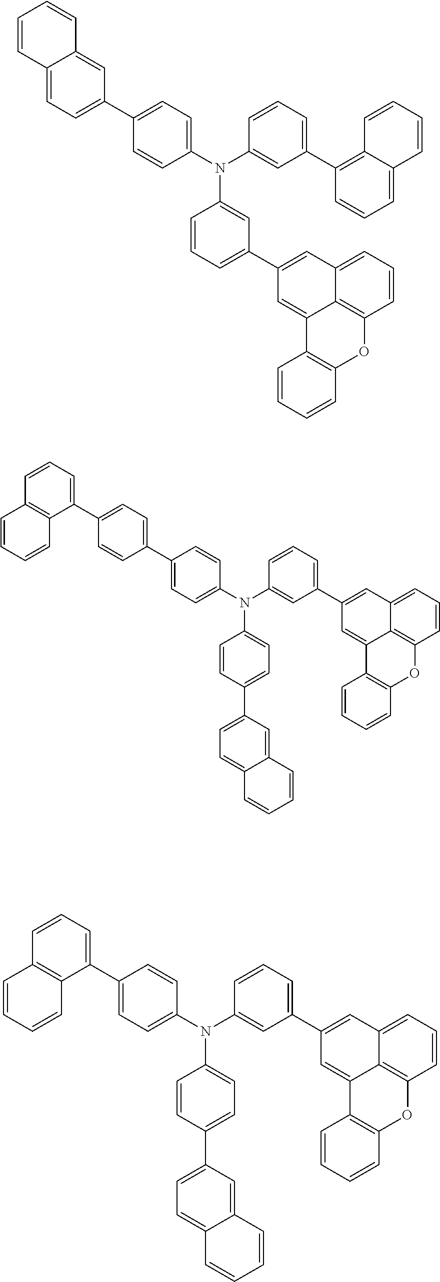 [SMILES: c1cc(-c2cc3c4c(cccc4c2)Oc2ccccc2-3)cc(N(c2ccc(-c3ccc4ccccc4c3)cc2)c2cccc(-c3cccc4ccccc34)c2)c1 ; c1cc(-c2cc3c4c(cccc4c2)Oc2ccccc2-3)cc(N(c2ccc(-c3ccc(-c4cccc5ccccc45)cc3)cc2)c2ccc(-c3ccc4ccccc4c3)cc2)c1 ; c1cc(-c2cc3c4c(cccc4c2)Oc2ccccc2-3)cc(N(c2ccc(-c3ccc4ccccc4c3)cc2)c2ccc(-c3cccc4ccccc34)cc2)c1]

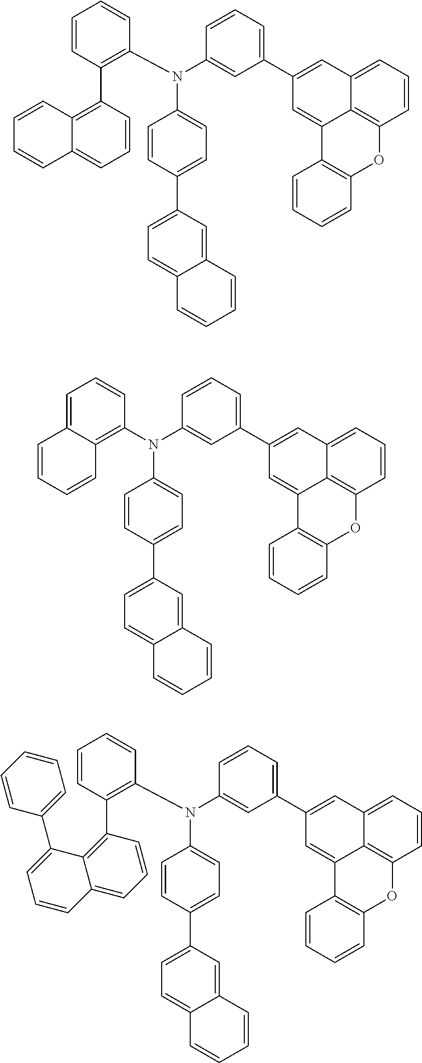 [SMILES: c1cc(-c2cc3c4c(cccc4c2)Oc2ccccc2-3)cc(N(c2ccc(-c3ccc4ccccc4c3)cc2)c2ccccc2-c2cccc3ccccc23)c1 ; c1cc(-c2cc3c4c(cccc4c2)Oc2ccccc2-3)cc(N(c2ccc(-c3ccc4ccccc4c3)cc2)c2cccc3ccccc23)c1 ; c1ccc(-c2cccc3cccc(-c4ccccc4N(c4ccc(-c5ccc6ccccc6c5)cc4)c4cccc(-c5cc6c7c(cccc7c5)Oc5ccccc5-6)c4)c23)cc1]

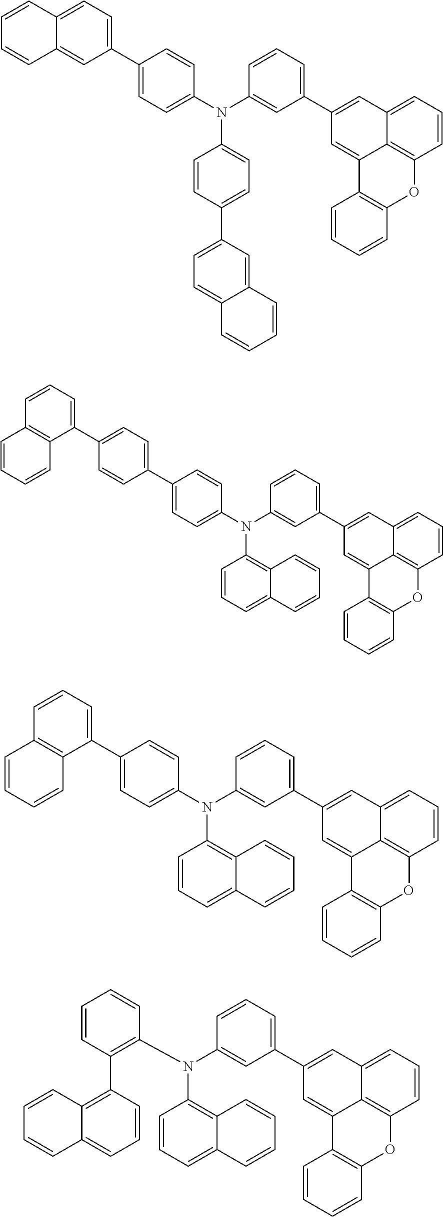 [SMILES: c1cc(-c2cc3c4c(cccc4c2)Oc2ccccc2-3)cc(N(c2ccc(-c3ccc4ccccc4c3)cc2)c2ccc(-c3ccc4ccccc4c3)cc2)c1 ; c1cc(-c2cc3c4c(cccc4c2)Oc2ccccc2-3)cc(N(c2ccc(-c3ccc(-c4cccc5ccccc45)cc3)cc2)c2cccc3ccccc23)c1 ; c1cc(-c2cc3c4c(cccc4c2)Oc2ccccc2-3)cc(N(c2ccc(-c3cccc4ccccc34)cc2)c2cccc3ccccc23)c1 ; c1cc(-c2cc3c4c(cccc4c2)Oc2ccccc2-3)cc(N(c2ccccc2-c2cccc3ccccc23)c2cccc3ccccc23)c1]

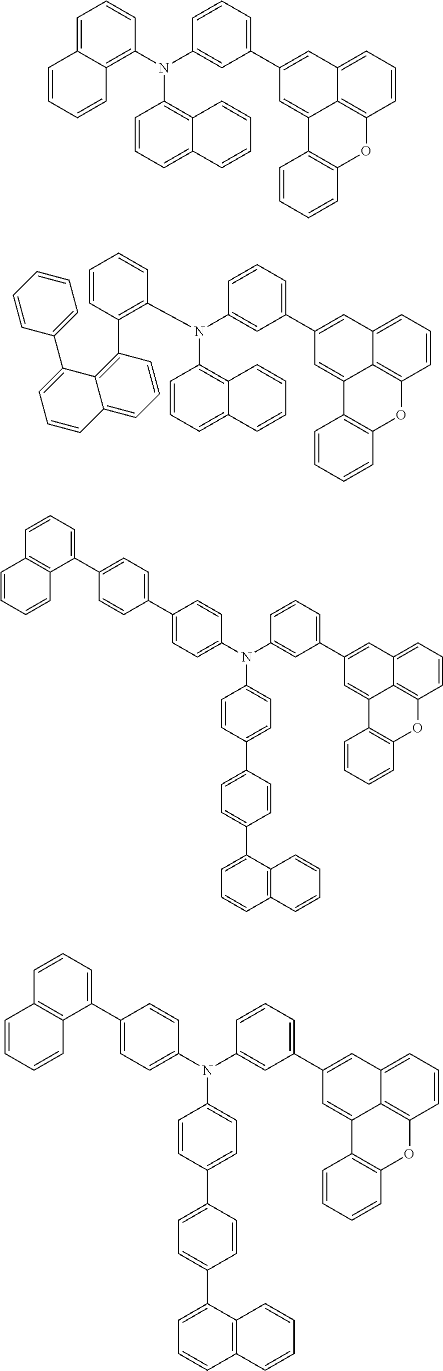 [SMILES: c1cc(-c2cc3c4c(cccc4c2)Oc2ccccc2-3)cc(N(c2cccc3ccccc23)c2cccc3ccccc23)c1 ; c1ccc(-c2cccc3cccc(-c4ccccc4N(c4cccc(-c5cc6c7c(cccc7c5)Oc5ccccc5-6)c4)c4cccc5ccccc45)c23)cc1 ; c1cc(-c2cc3c4c(cccc4c2)Oc2ccccc2-3)cc(N(c2ccc(-c3ccc(-c4cccc5ccccc45)cc3)cc2)c2ccc(-c3ccc(-c4cccc5ccccc45)cc3)cc2)c1 ; c1cc(-c2cc3c4c(cccc4c2)Oc2ccccc2-3)cc(N(c2ccc(-c3ccc(-c4cccc5ccccc45)cc3)cc2)c2ccc(-c3cccc4ccccc34)cc2)c1]

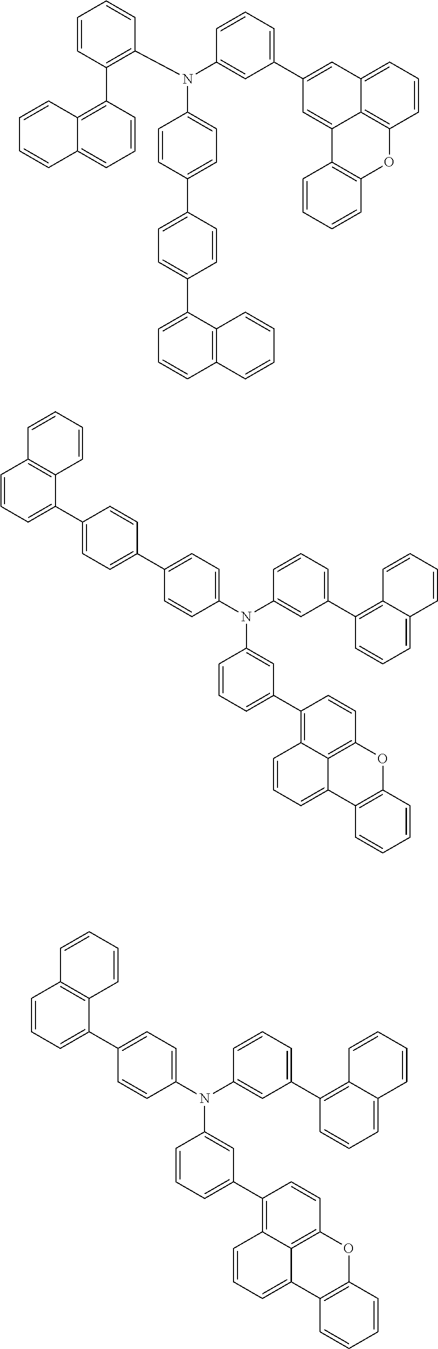 [SMILES: c1cc(-c2cc3c4c(cccc4c2)Oc2ccccc2-3)cc(N(c2ccc(-c3ccc(-c4cccc5ccccc45)cc3)cc2)c2ccccc2-c2cccc3ccccc23)c1 ; c1cc(-c2cccc3ccccc23)cc(N(c2ccc(-c3ccc(-c4cccc5ccccc45)cc3)cc2)c2cccc(-c3ccc4c5c(cccc35)-c3ccccc3O4)c2)c1 ; c1cc(-c2cccc3ccccc23)cc(N(c2ccc(-c3cccc4ccccc34)cc2)c2cccc(-c3ccc4c5c(cccc35)-c3ccccc3O4)c2)c1]

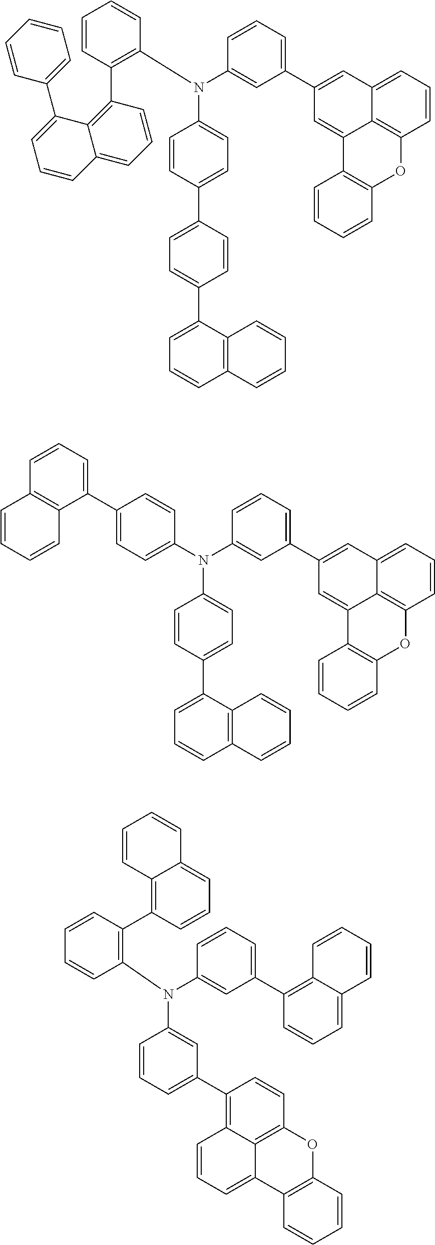 [SMILES: c1ccc(-c2cccc3cccc(-c4ccccc4N(c4ccc(-c5ccc(-c6cccc7ccccc67)cc5)cc4)c4cccc(-c5cc6c7c(cccc7c5)Oc5ccccc5-6)c4)c23)cc1 ; c1cc(-c2cc3c4c(cccc4c2)Oc2ccccc2-3)cc(N(c2ccc(-c3cccc4ccccc34)cc2)c2ccc(-c3cccc4ccccc34)cc2)c1 ; c1cc(-c2cccc3ccccc23)cc(N(c2cccc(-c3ccc4c5c(cccc35)-c3ccccc3O4)c2)c2ccccc2-c2cccc3ccccc23)c1]

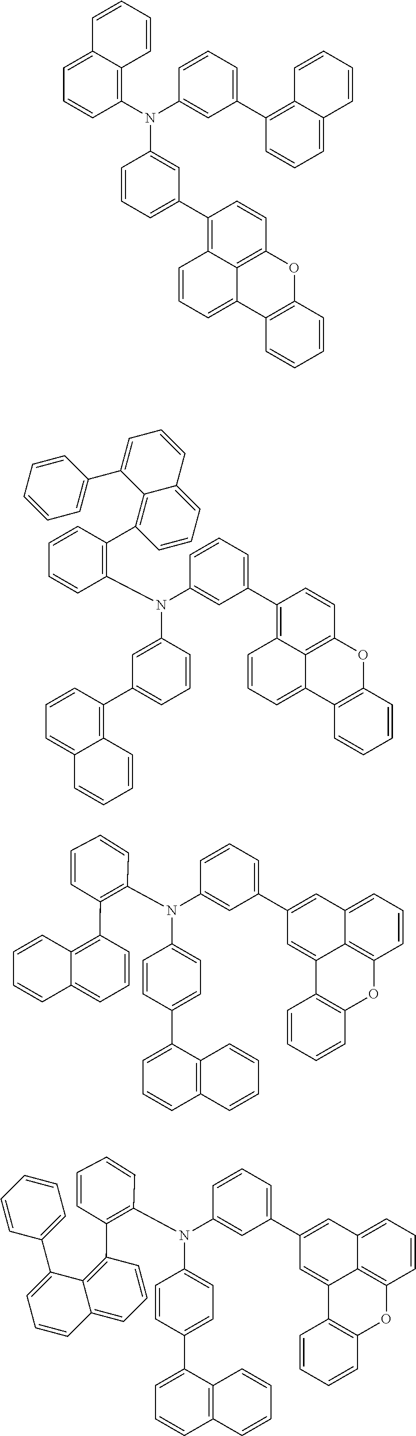

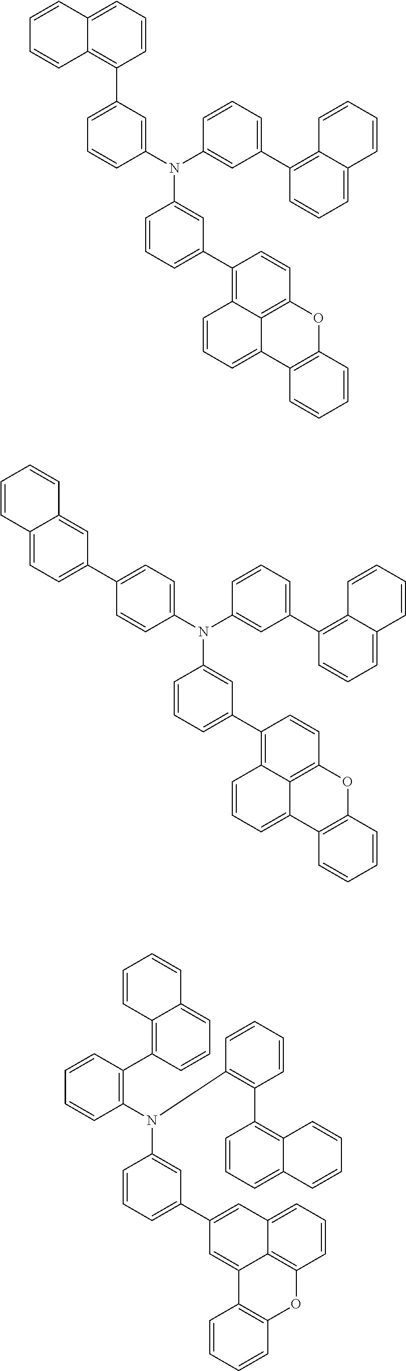 [SMILES: c1cc(-c2cccc3ccccc23)cc(N(c2cccc(-c3cccc4ccccc34)c2)c2cccc(-c3ccc4c5c(cccc35)-c3ccccc3O4)c2)c1 ; c1cc(-c2cccc3ccccc23)cc(N(c2ccc(-c3ccc4ccccc4c3)cc2)c2cccc(-c3ccc4c5c(cccc35)-c3ccccc3O4)c2)c1 ; c1cc(-c2cc3c4c(cccc4c2)Oc2ccccc2-3)cc(N(c2ccccc2-c2cccc3ccccc23)c2ccccc2-c2cccc3ccccc23)c1]

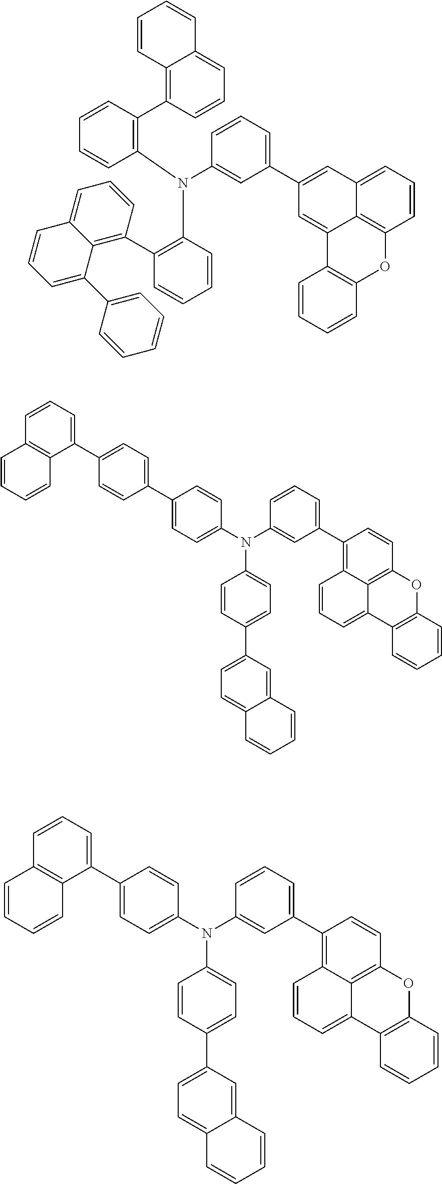 [SMILES: c1ccc(-c2cccc3cccc(-c4ccccc4N(c4cccc(-c5cc6c7c(cccc7c5)Oc5ccccc5-6)c4)c4ccccc4-c4cccc5ccccc45)c23)cc1 ; c1cc(-c2ccc3c4c(cccc24)-c2ccccc2O3)cc(N(c2ccc(-c3ccc(-c4cccc5ccccc45)cc3)cc2)c2ccc(-c3ccc4ccccc4c3)cc2)c1 ; c1cc(-c2ccc3c4c(cccc24)-c2ccccc2O3)cc(N(c2ccc(-c3ccc4ccccc4c3)cc2)c2ccc(-c3cccc4ccccc34)cc2)c1]

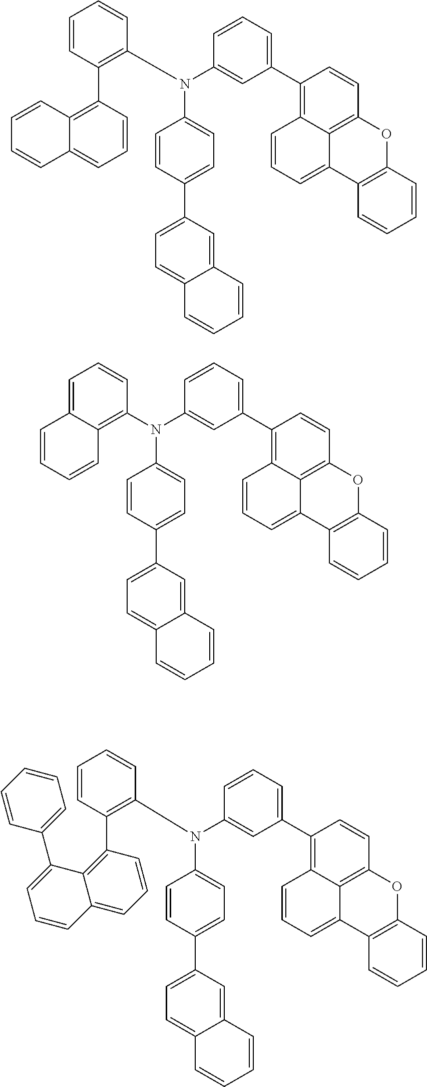

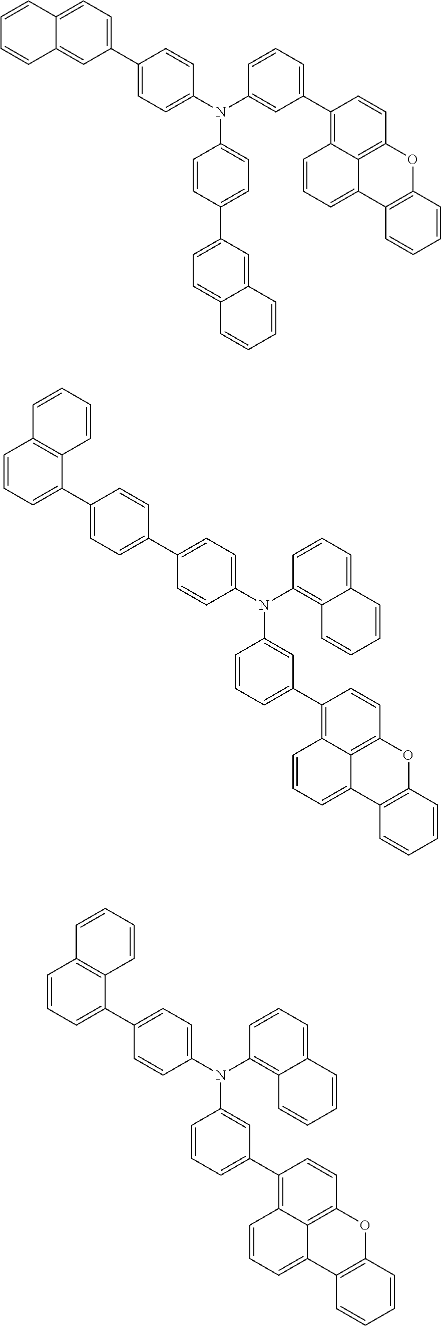 [SMILES: c1cc(-c2ccc3c4c(cccc24)-c2ccccc2O3)cc(N(c2ccc(-c3ccc4ccccc4c3)cc2)c2ccc(-c3ccc4ccccc4c3)cc2)c1 ; c1cc(-c2ccc3c4c(cccc24)-c2ccccc2O3)cc(N(c2ccc(-c3ccc(-c4cccc5ccccc45)cc3)cc2)c2cccc3ccccc23)c1 ; c1cc(-c2ccc3c4c(cccc24)-c2ccccc2O3)cc(N(c2ccc(-c3cccc4ccccc34)cc2)c2cccc3ccccc23)c1]

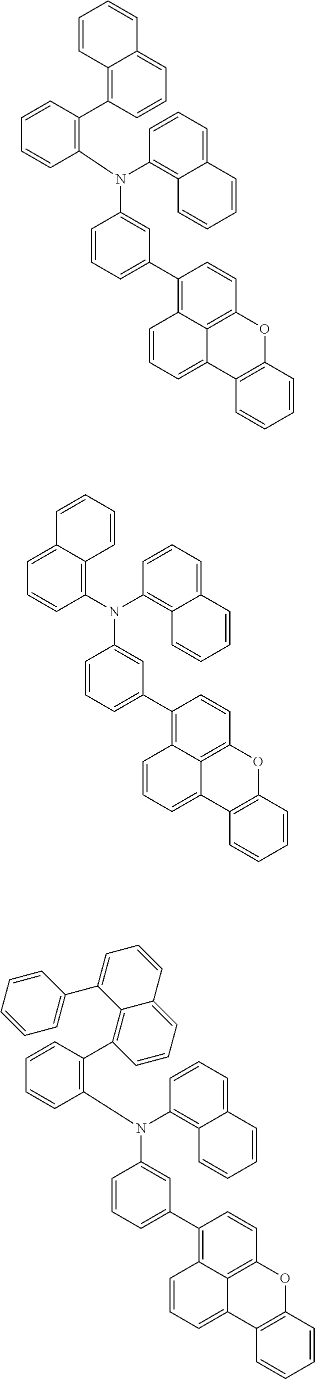 [SMILES: c1cc(-c2ccc3c4c(cccc24)-c2ccccc2O3)cc(N(c2ccccc2-c2cccc3ccccc23)c2cccc3ccccc23)c1 ; c1cc(-c2ccc3c4c(cccc24)-c2ccccc2O3)cc(N(c2cccc3ccccc23)c2cccc3ccccc23)c1 ; c1ccc(-c2cccc3cccc(-c4ccccc4N(c4cccc(-c5ccc6c7c(cccc57)-c5ccccc5O6)c4)c4cccc5ccccc45)c23)cc1]

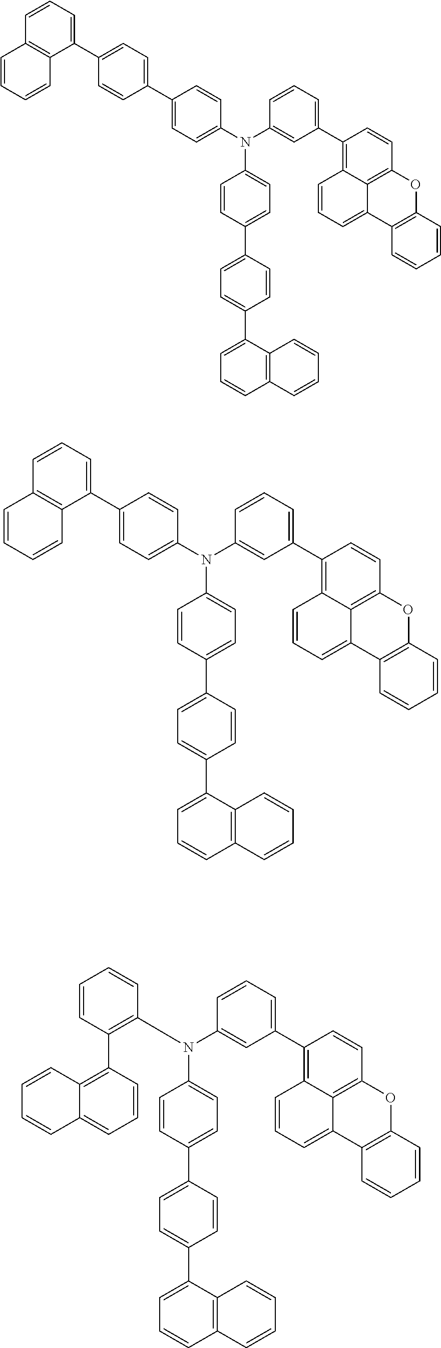 [SMILES: c1cc(-c2ccc3c4c(cccc24)-c2ccccc2O3)cc(N(c2ccc(-c3ccc(-c4cccc5ccccc45)cc3)cc2)c2ccc(-c3ccc(-c4cccc5ccccc45)cc3)cc2)c1 ; c1cc(-c2ccc3c4c(cccc24)-c2ccccc2O3)cc(N(c2ccc(-c3ccc(-c4cccc5ccccc45)cc3)cc2)c2ccc(-c3cccc4ccccc34)cc2)c1 ; c1cc(-c2ccc3c4c(cccc24)-c2ccccc2O3)cc(N(c2ccc(-c3ccc(-c4cccc5ccccc45)cc3)cc2)c2ccccc2-c2cccc3ccccc23)c1]

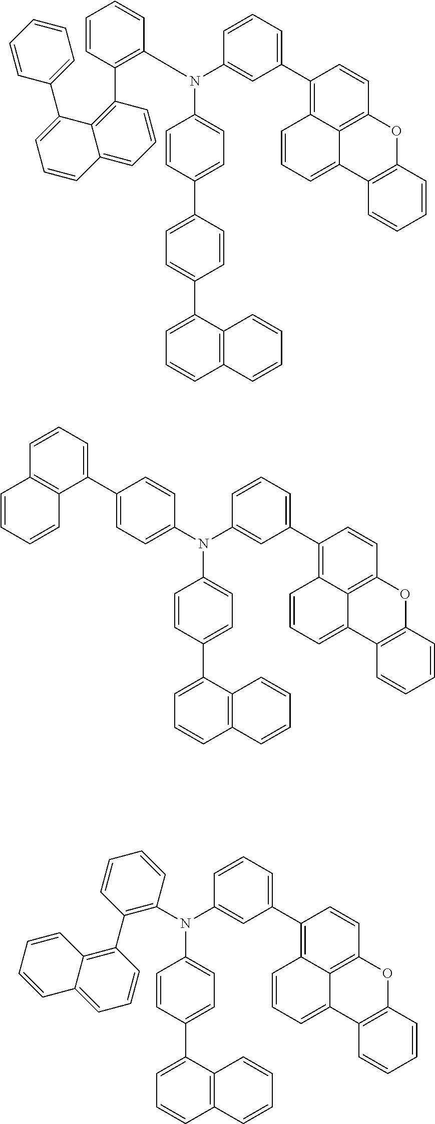 [SMILES: c1ccc(-c2cccc3cccc(-c4ccccc4N(c4ccc(-c5ccc(-c6cccc7ccccc67)cc5)cc4)c4cccc(-c5ccc6c7c(cccc57)-c5ccccc5O6)c4)c23)cc1 ; c1cc(-c2ccc3c4c(cccc24)-c2ccccc2O3)cc(N(c2ccc(-c3cccc4ccccc34)cc2)c2ccc(-c3cccc4ccccc34)cc2)c1 ; c1cc(-c2ccc3c4c(cccc24)-c2ccccc2O3)cc(N(c2ccc(-c3cccc4ccccc34)cc2)c2ccccc2-c2cccc3ccccc23)c1]

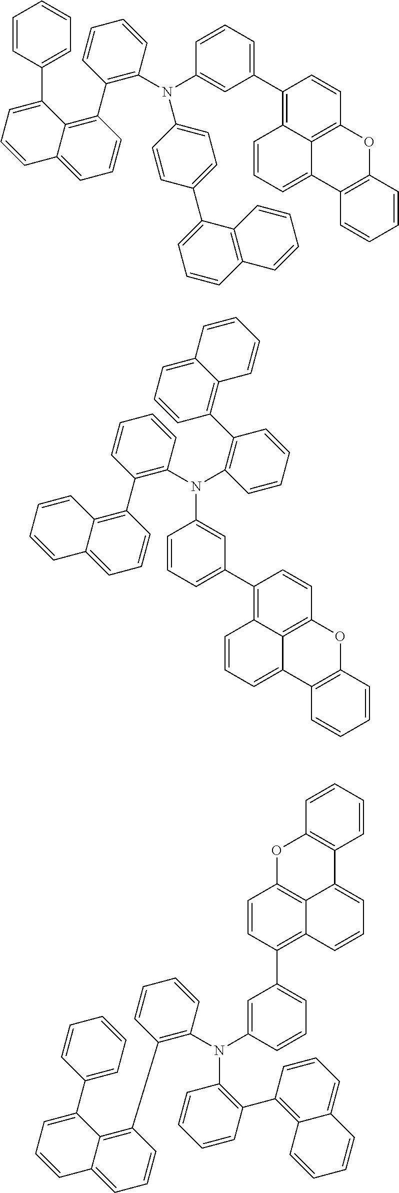

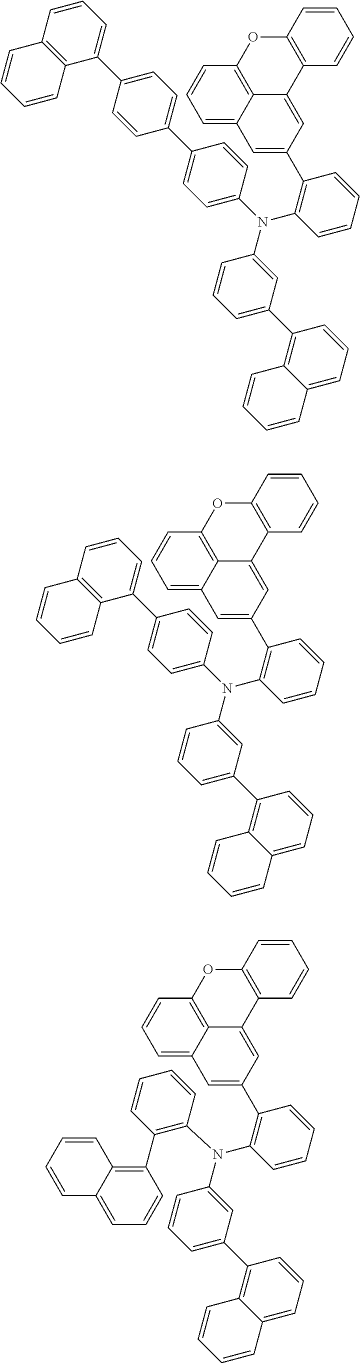

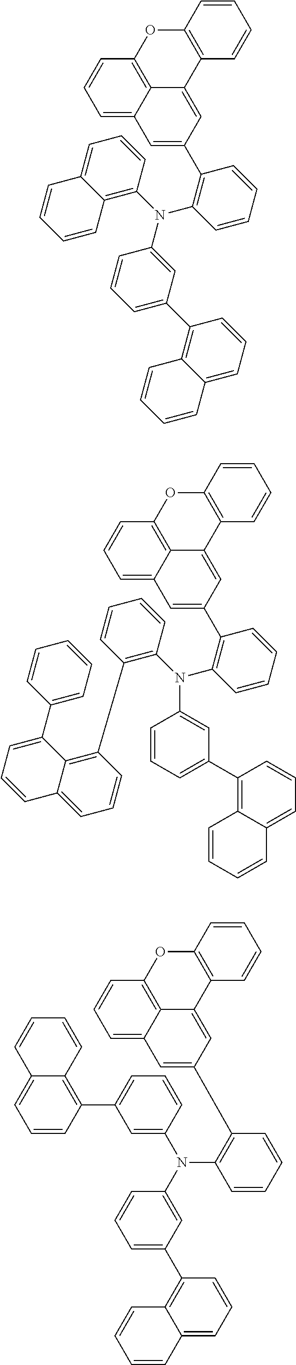

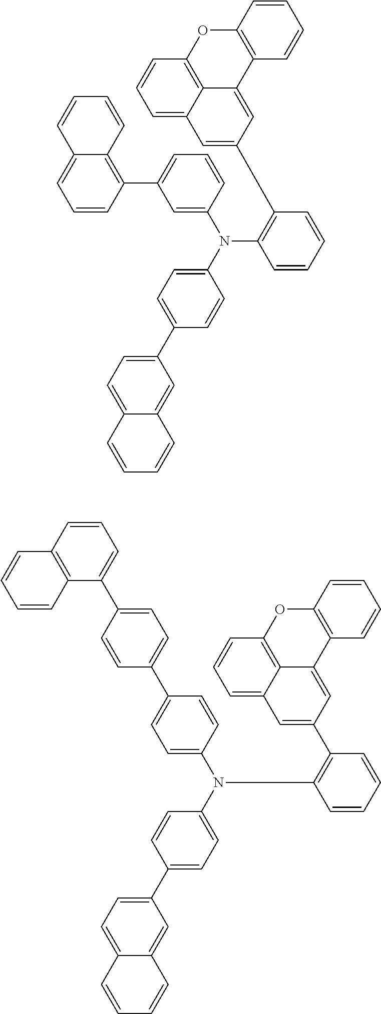 [SMILES: c1cc(-c2cccc3ccccc23)cc(N(c2ccc(-c3ccc4ccccc4c3)cc2)c2ccccc2-c2cc3c4c(cccc4c2)Oc2ccccc2-3)c1 ; c1ccc2c(c1)Oc1cccc3cc(-c4ccccc4N(c4ccc(-c5ccc(-c6cccc7ccccc67)cc5)cc4)c4ccc(-c5ccc6ccccc6c5)cc4)cc-2c13]

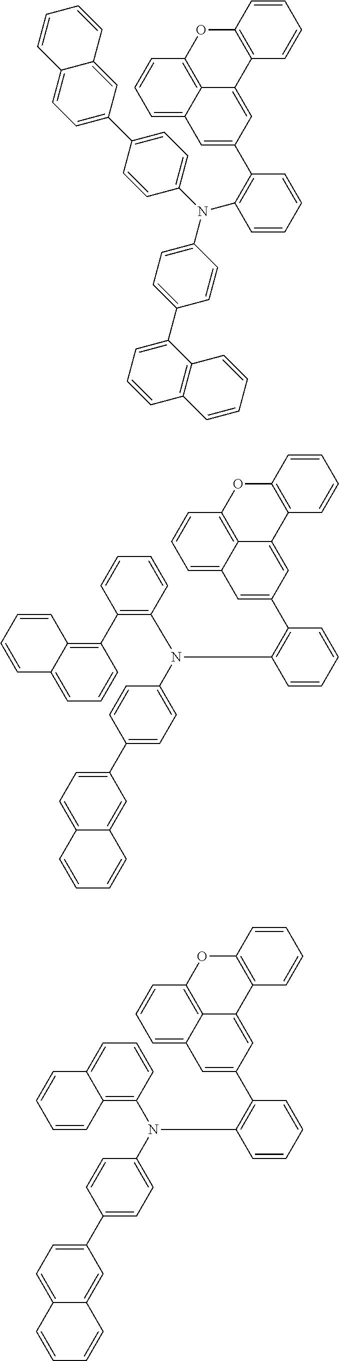 [SMILES: c1ccc2c(c1)Oc1cccc3cc(-c4ccccc4N(c4ccc(-c5ccc6ccccc6c5)cc4)c4ccc(-c5cccc6ccccc56)cc4)cc-2c13 ; c1ccc2c(c1)Oc1cccc3cc(-c4ccccc4N(c4ccc(-c5ccc6ccccc6c5)cc4)c4ccccc4-c4cccc5ccccc45)cc-2c13 ; c1ccc2c(c1)Oc1cccc3cc(-c4ccccc4N(c4ccc(-c5ccc6ccccc6c5)cc4)c4cccc5ccccc45)cc-2c13]

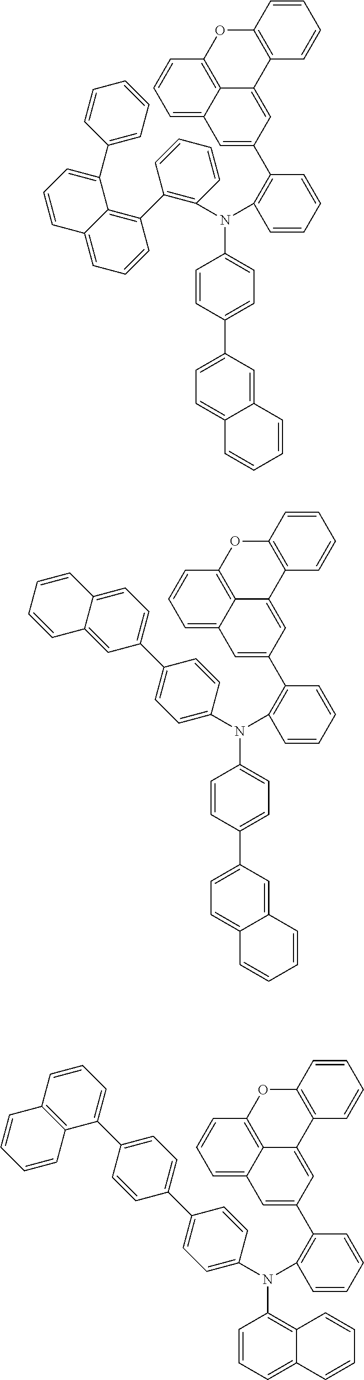 [SMILES: c1ccc(-c2cccc3cccc(-c4ccccc4N(c4ccc(-c5ccc6ccccc6c5)cc4)c4ccccc4-c4cc5c6c(cccc6c4)Oc4ccccc4-5)c23)cc1 ; c1ccc2c(c1)Oc1cccc3cc(-c4ccccc4N(c4ccc(-c5ccc6ccccc6c5)cc4)c4ccc(-c5ccc6ccccc6c5)cc4)cc-2c13 ; c1ccc2c(c1)Oc1cccc3cc(-c4ccccc4N(c4ccc(-c5ccc(-c6cccc7ccccc67)cc5)cc4)c4cccc5ccccc45)cc-2c13]

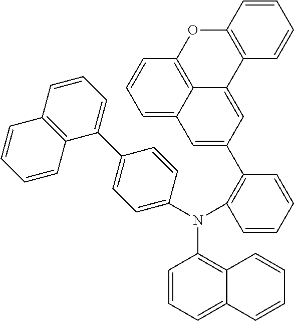 [SMILES: c1ccc2c(c1)Oc1cccc3cc(-c4ccccc4N(c4ccc(-c5cccc6ccccc56)cc4)c4cccc5ccccc45)cc-2c13]

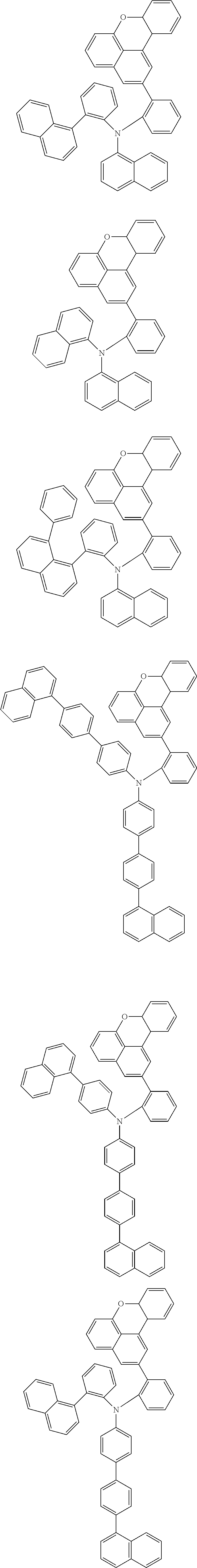

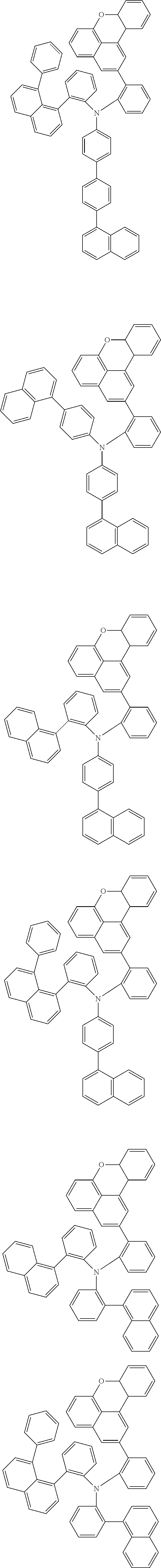

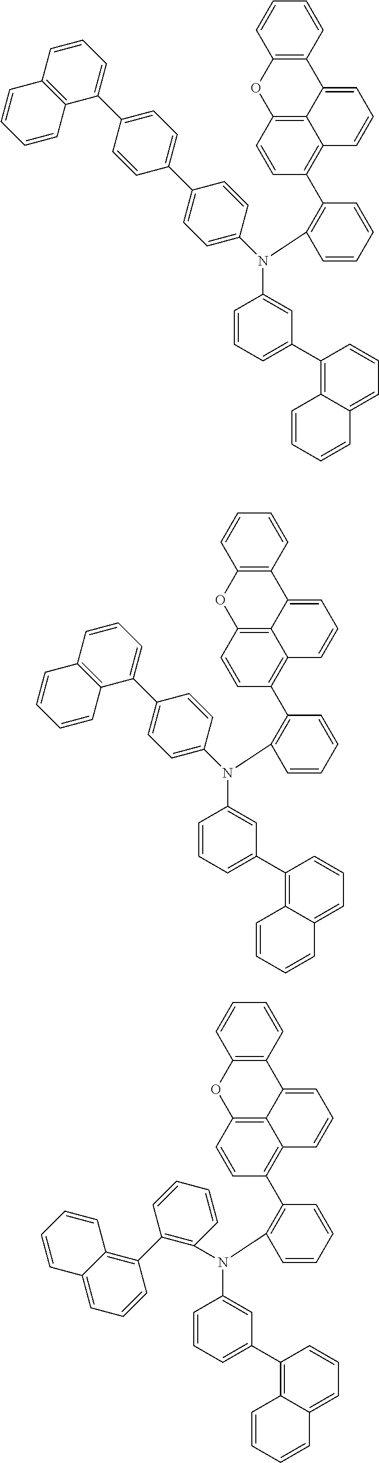

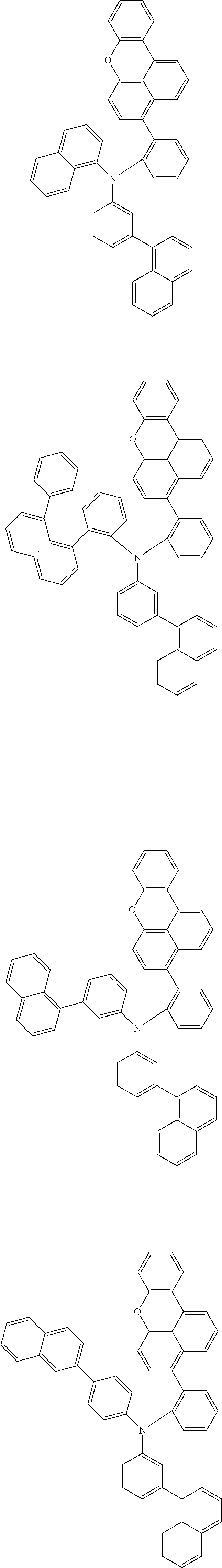 [SMILES: c1cc(-c2cccc3ccccc23)cc(N(c2ccccc2-c2ccc3c4c(cccc24)-c2ccccc2O3)c2cccc3ccccc23)c1 ; c1ccc(-c2cccc3cccc(-c4ccccc4N(c4cccc(-c5cccc6ccccc56)c4)c4ccccc4-c4ccc5c6c(cccc46)-c4ccccc4O5)c23)cc1 ; c1cc(-c2cccc3ccccc23)cc(N(c2cccc(-c3cccc4ccccc34)c2)c2ccccc2-c2ccc3c4c(cccc24)-c2ccccc2O3)c1 ; c1cc(-c2cccc3ccccc23)cc(N(c2ccc(-c3ccc4ccccc4c3)cc2)c2ccccc2-c2ccc3c4c(cccc24)-c2ccccc2O3)c1]

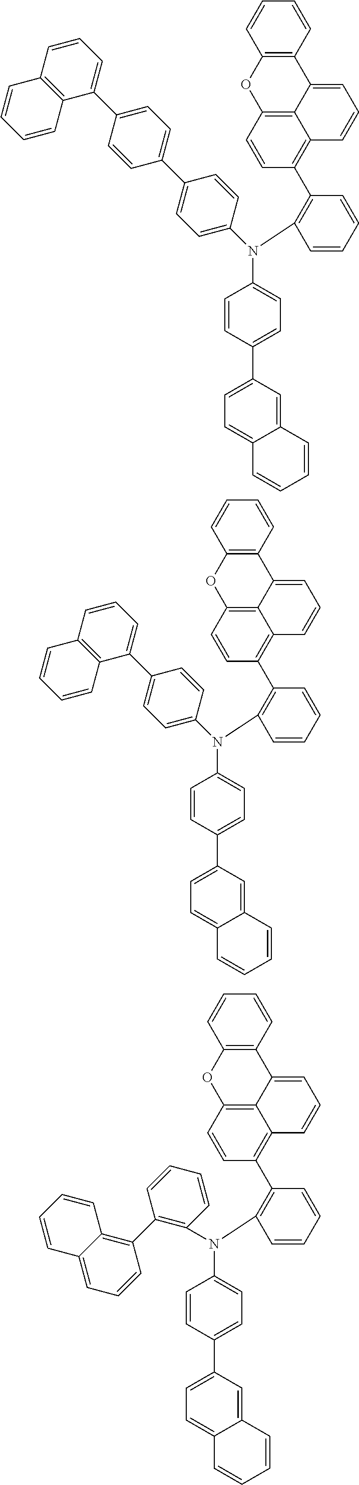

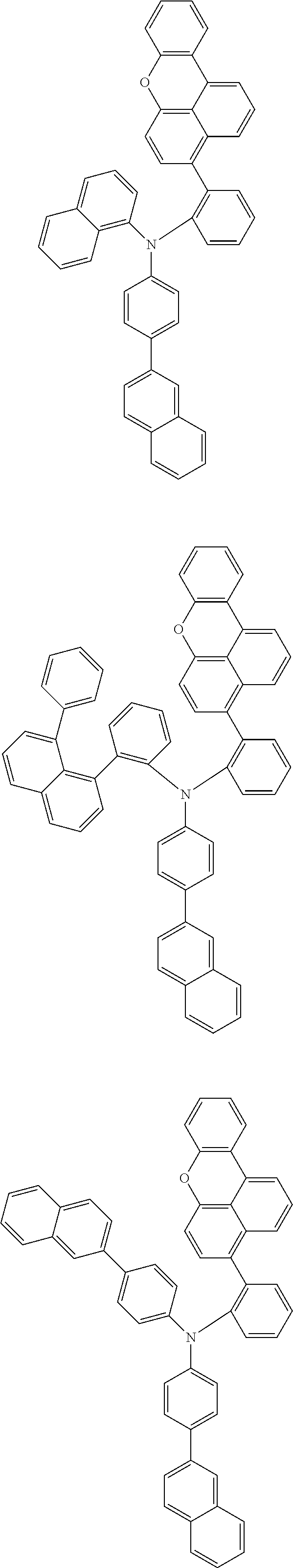 [SMILES: c1ccc2c(c1)Oc1ccc(-c3ccccc3N(c3ccc(-c4ccc5ccccc5c4)cc3)c3cccc4ccccc34)c3cccc-2c13 ; c1ccc(-c2cccc3cccc(-c4ccccc4N(c4ccc(-c5ccc6ccccc6c5)cc4)c4ccccc4-c4ccc5c6c(cccc46)-c4ccccc4O5)c23)cc1 ; c1ccc2c(c1)Oc1ccc(-c3ccccc3N(c3ccc(-c4ccc5ccccc5c4)cc3)c3ccc(-c4ccc5ccccc5c4)cc3)c3cccc-2c13]

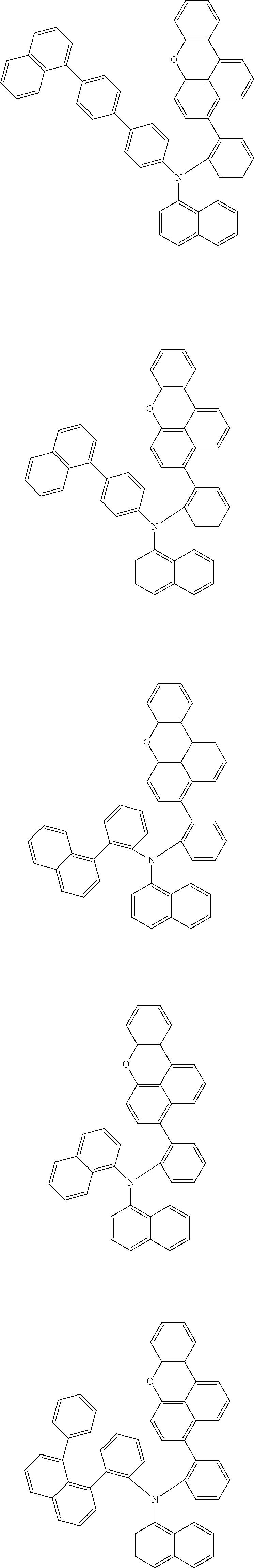 [SMILES: c1ccc2c(c1)Oc1ccc(-c3ccccc3N(c3ccc(-c4ccc(-c5cccc6ccccc56)cc4)cc3)c3cccc4ccccc34)c3cccc-2c13 ; c1ccc2c(c1)Oc1ccc(-c3ccccc3N(c3ccc(-c4cccc5ccccc45)cc3)c3cccc4ccccc34)c3cccc-2c13 ; c1ccc2c(c1)Oc1ccc(-c3ccccc3N(c3ccccc3-c3cccc4ccccc34)c3cccc4ccccc34)c3cccc-2c13 ; c1ccc2c(c1)Oc1ccc(-c3ccccc3N(c3cccc4ccccc34)c3cccc4ccccc34)c3cccc-2c13 ; c1ccc(-c2cccc3cccc(-c4ccccc4N(c4ccccc4-c4ccc5c6c(cccc46)-c4ccccc4O5)c4cccc5ccccc45)c23)cc1]

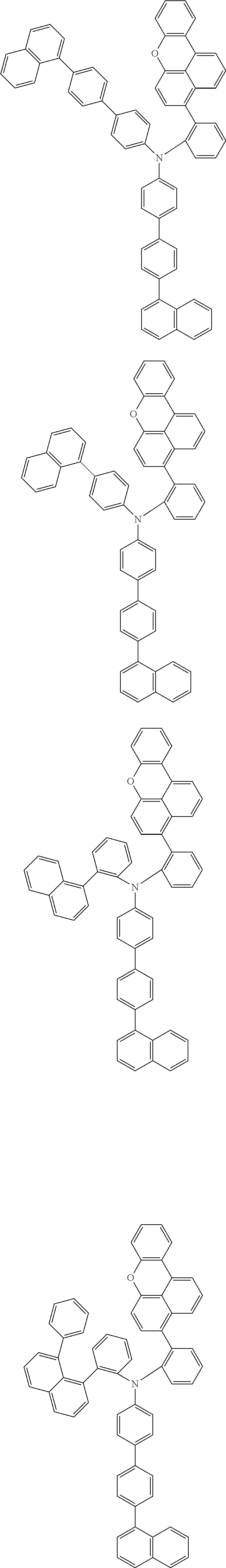

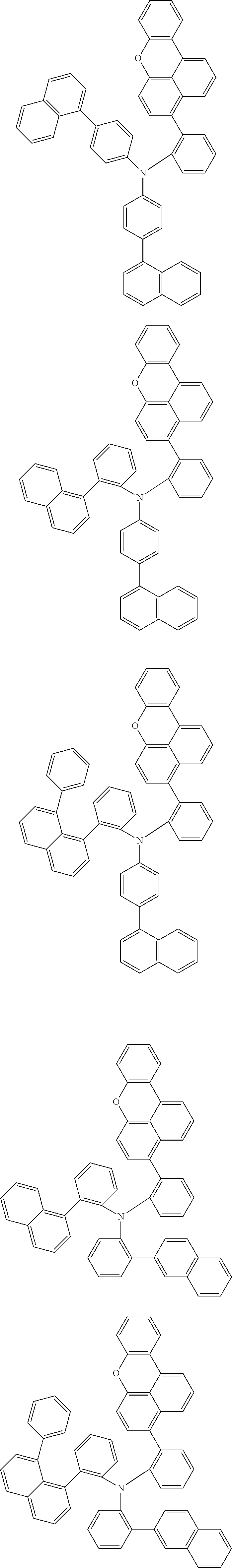

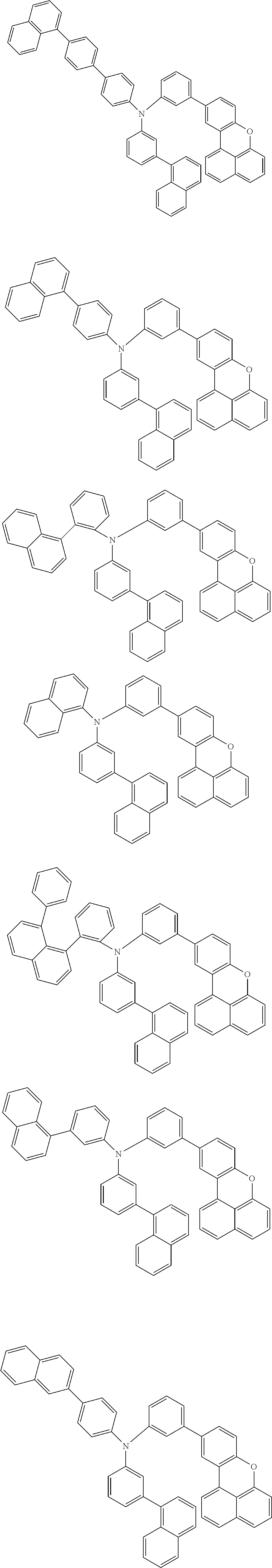

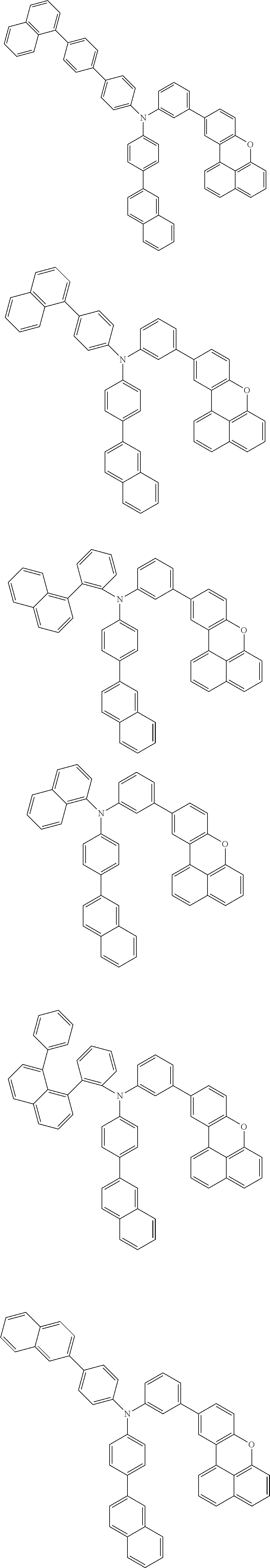

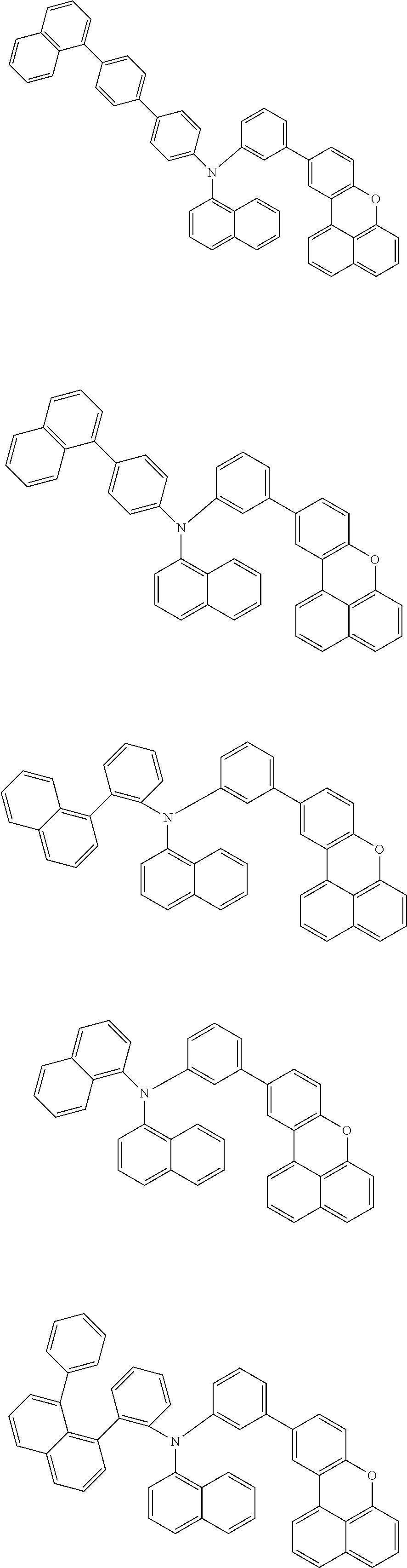 [SMILES: c1cc(-c2ccc3c(c2)-c2cccc4cccc(c24)O3)cc(N(c2ccc(-c3ccc(-c4cccc5ccccc45)cc3)cc2)c2cccc3ccccc23)c1 ; c1cc(-c2ccc3c(c2)-c2cccc4cccc(c24)O3)cc(N(c2ccc(-c3cccc4ccccc34)cc2)c2cccc3ccccc23)c1 ; c1cc(-c2ccc3c(c2)-c2cccc4cccc(c24)O3)cc(N(c2ccccc2-c2cccc3ccccc23)c2cccc3ccccc23)c1 ; c1cc(-c2ccc3c(c2)-c2cccc4cccc(c24)O3)cc(N(c2cccc3ccccc23)c2cccc3ccccc23)c1 ; c1ccc(-c2cccc3cccc(-c4ccccc4N(c4cccc(-c5ccc6c(c5)-c5cccc7cccc(c57)O6)c4)c4cccc5ccccc45)c23)cc1]

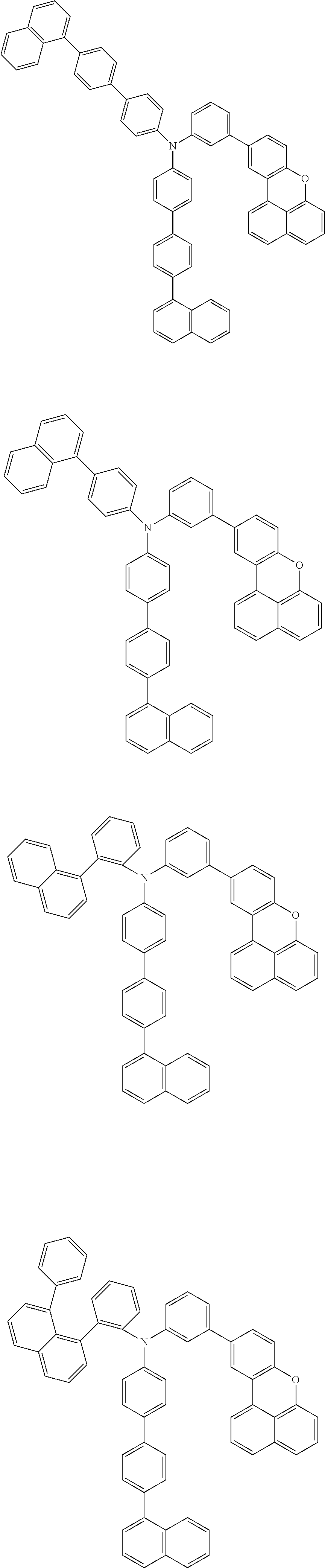 [SMILES: c1cc(-c2ccc3c(c2)-c2cccc4cccc(c24)O3)cc(N(c2ccc(-c3ccc(-c4cccc5ccccc45)cc3)cc2)c2ccc(-c3ccc(-c4cccc5ccccc45)cc3)cc2)c1 ; c1cc(-c2ccc3c(c2)-c2cccc4cccc(c24)O3)cc(N(c2ccc(-c3ccc(-c4cccc5ccccc45)cc3)cc2)c2ccc(-c3cccc4ccccc34)cc2)c1 ; c1cc(-c2ccc3c(c2)-c2cccc4cccc(c24)O3)cc(N(c2ccc(-c3ccc(-c4cccc5ccccc45)cc3)cc2)c2ccccc2-c2cccc3ccccc23)c1 ; c1ccc(-c2cccc3cccc(-c4ccccc4N(c4ccc(-c5ccc(-c6cccc7ccccc67)cc5)cc4)c4cccc(-c5ccc6c(c5)-c5cccc7cccc(c57)O6)c4)c23)cc1]

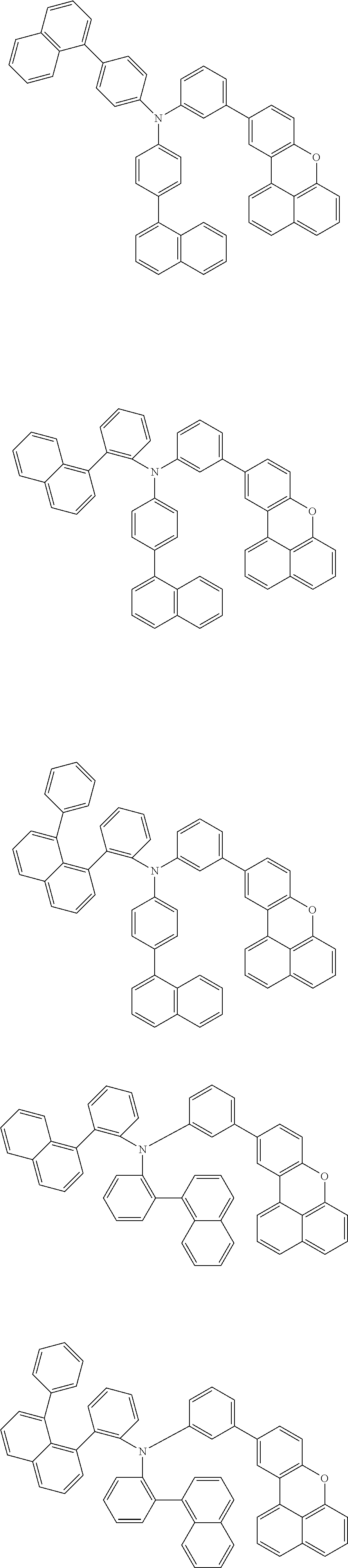

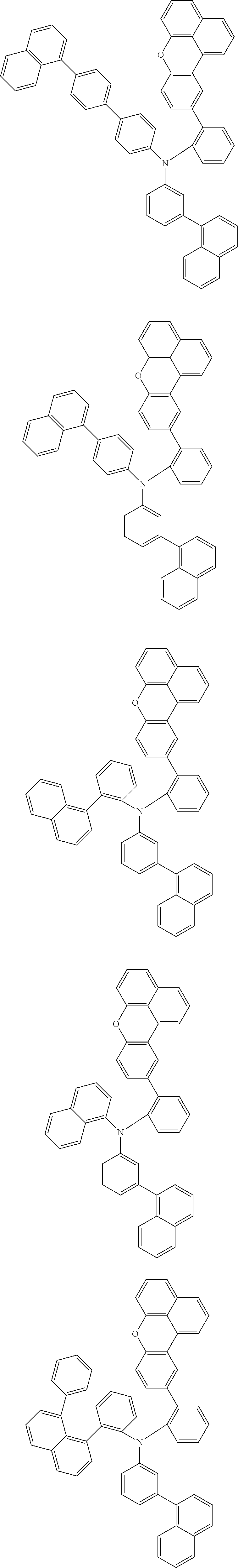

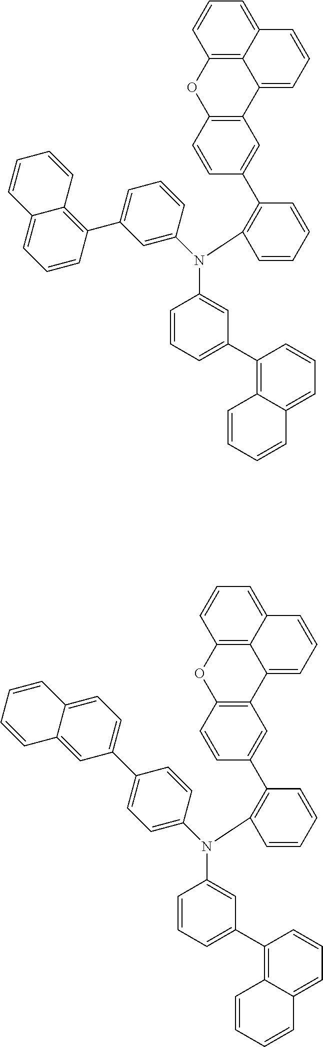 [SMILES: c1cc(-c2cccc3ccccc23)cc(N(c2cccc(-c3cccc4ccccc34)c2)c2ccccc2-c2ccc3c(c2)-c2cccc4cccc(c24)O3)c1 ; c1cc(-c2cccc3ccccc23)cc(N(c2ccc(-c3ccc4ccccc4c3)cc2)c2ccccc2-c2ccc3c(c2)-c2cccc4cccc(c24)O3)c1]

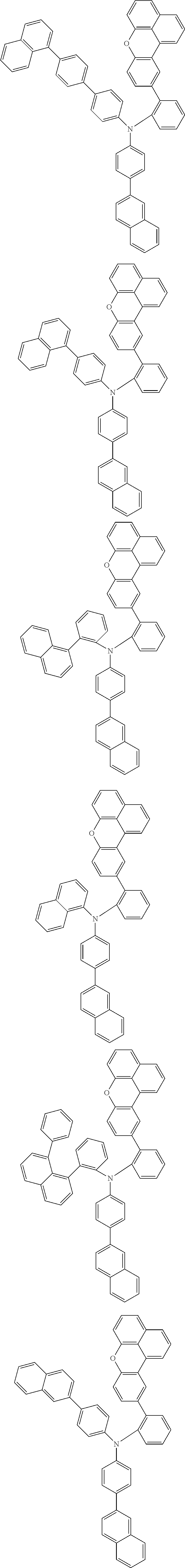

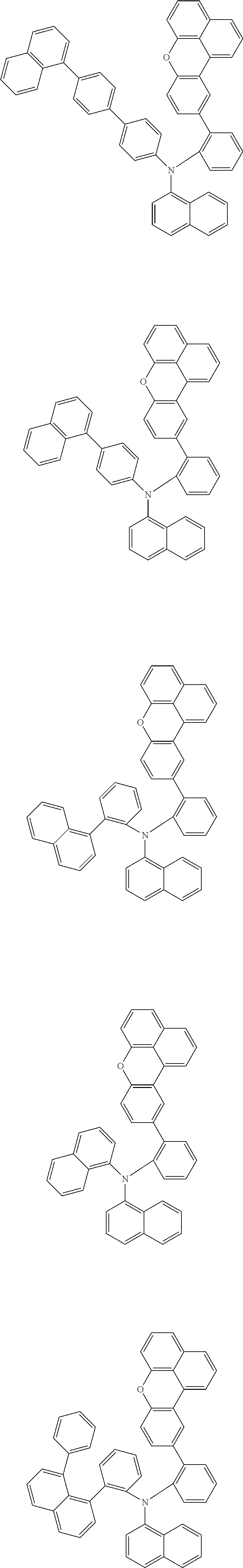 [SMILES: c1ccc(N(c2ccc(-c3ccc(-c4cccc5ccccc45)cc3)cc2)c2cccc3ccccc23)c(-c2ccc3c(c2)-c2cccc4cccc(c24)O3)c1 ; c1ccc(N(c2ccc(-c3cccc4ccccc34)cc2)c2cccc3ccccc23)c(-c2ccc3c(c2)-c2cccc4cccc(c24)O3)c1 ; c1ccc(N(c2ccccc2-c2cccc3ccccc23)c2cccc3ccccc23)c(-c2ccc3c(c2)-c2cccc4cccc(c24)O3)c1 ; c1ccc(N(c2cccc3ccccc23)c2cccc3ccccc23)c(-c2ccc3c(c2)-c2cccc4cccc(c24)O3)c1 ; c1ccc(-c2cccc3cccc(-c4ccccc4N(c4ccccc4-c4ccc5c(c4)-c4cccc6cccc(c46)O5)c4cccc5ccccc45)c23)cc1]

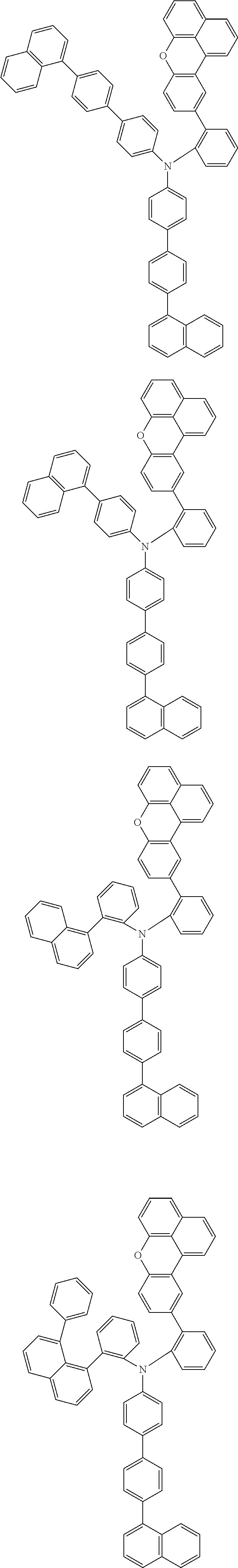

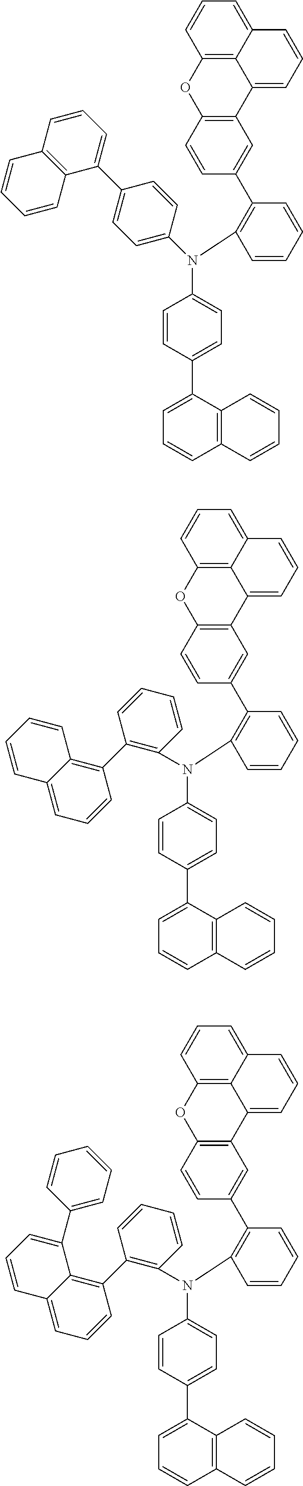 [SMILES: c1ccc(N(c2ccc(-c3cccc4ccccc34)cc2)c2ccc(-c3cccc4ccccc34)cc2)c(-c2ccc3c(c2)-c2cccc4cccc(c24)O3)c1 ; c1ccc(N(c2ccc(-c3cccc4ccccc34)cc2)c2ccccc2-c2cccc3ccccc23)c(-c2ccc3c(c2)-c2cccc4cccc(c24)O3)c1 ; c1ccc(-c2cccc3cccc(-c4ccccc4N(c4ccc(-c5cccc6ccccc56)cc4)c4ccccc4-c4ccc5c(c4)-c4cccc6cccc(c46)O5)c23)cc1]

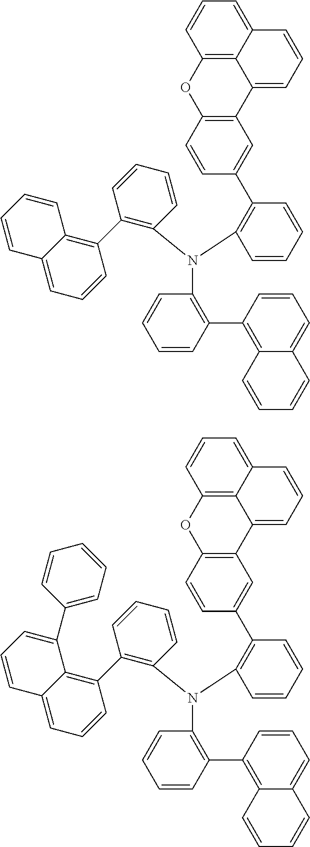

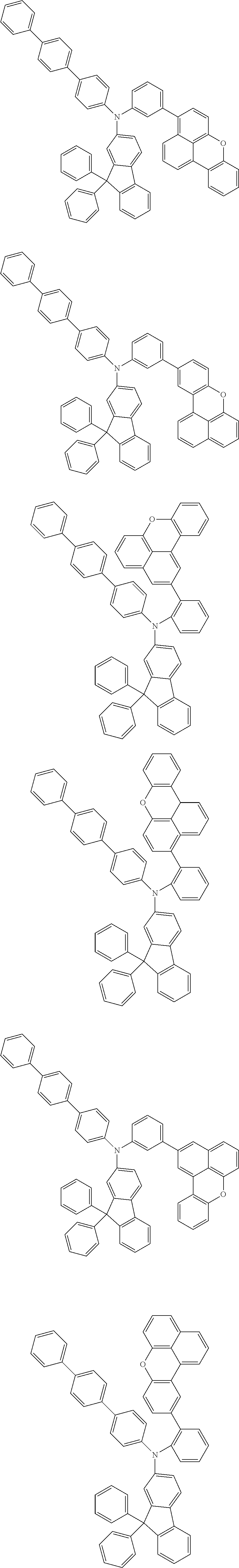

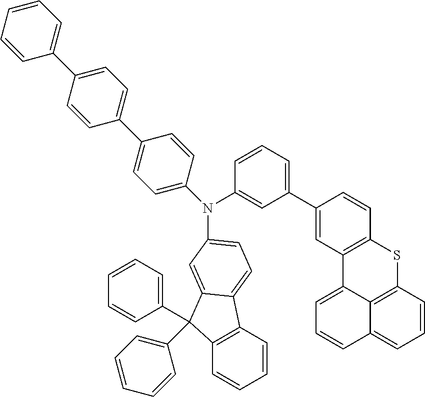 [SMILES: c1ccc(-c2ccc(-c3ccc(N(c4cccc(-c5ccc6c(c5)-c5cccc7cccc(c57)S6)c4)c4ccc5c(c4)C(c4ccccc4)(c4ccccc4)c4ccccc4-5)cc3)cc2)cc1]

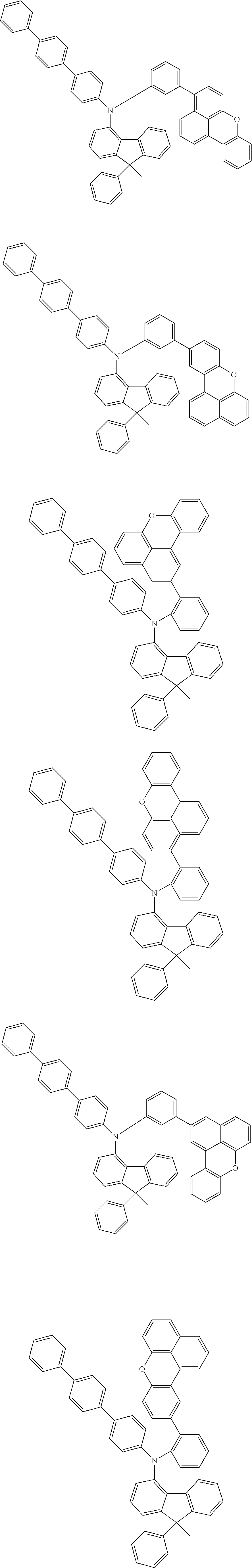

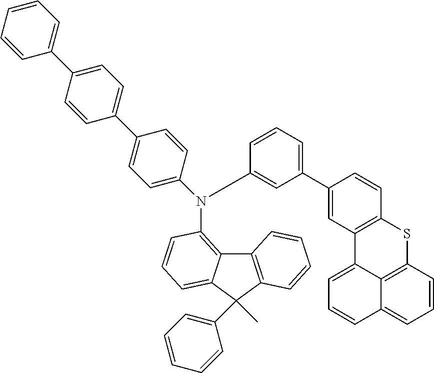 [SMILES: CC1(c2ccccc2)c2ccccc2-c2c(N(c3ccc(-c4ccc(-c5ccccc5)cc4)cc3)c3cccc(-c4ccc5c(c4)-c4cccc6cccc(c46)S5)c3)cccc21]

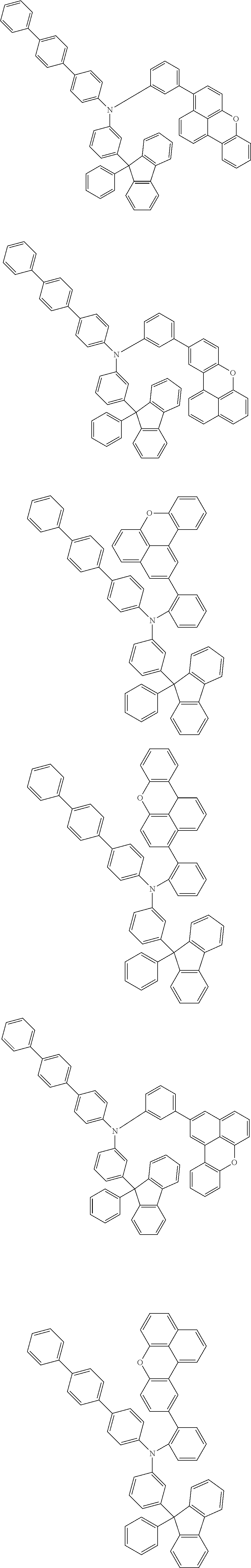

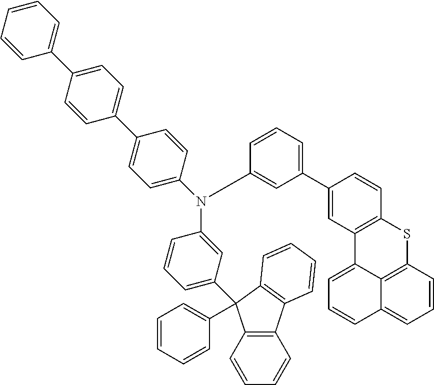 [SMILES: c1ccc(-c2ccc(-c3ccc(N(c4cccc(-c5ccc6c(c5)-c5cccc7cccc(c57)S6)c4)c4cccc(C5(c6ccccc6)c6ccccc6-c6ccccc65)c4)cc3)cc2)cc1]

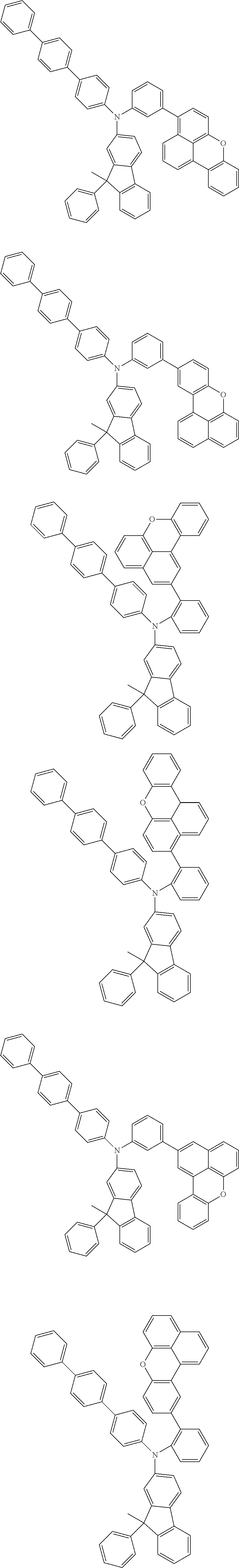

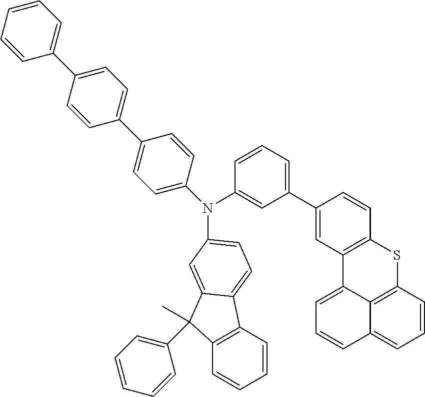 [SMILES: CC1(c2ccccc2)c2ccccc2-c2ccc(N(c3ccc(-c4ccc(-c5ccccc5)cc4)cc3)c3cccc(-c4ccc5c(c4)-c4cccc6cccc(c46)S5)c3)cc21]

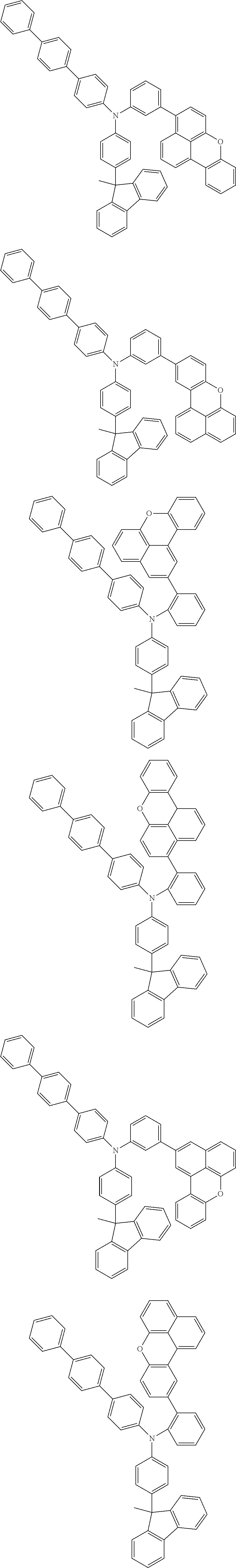

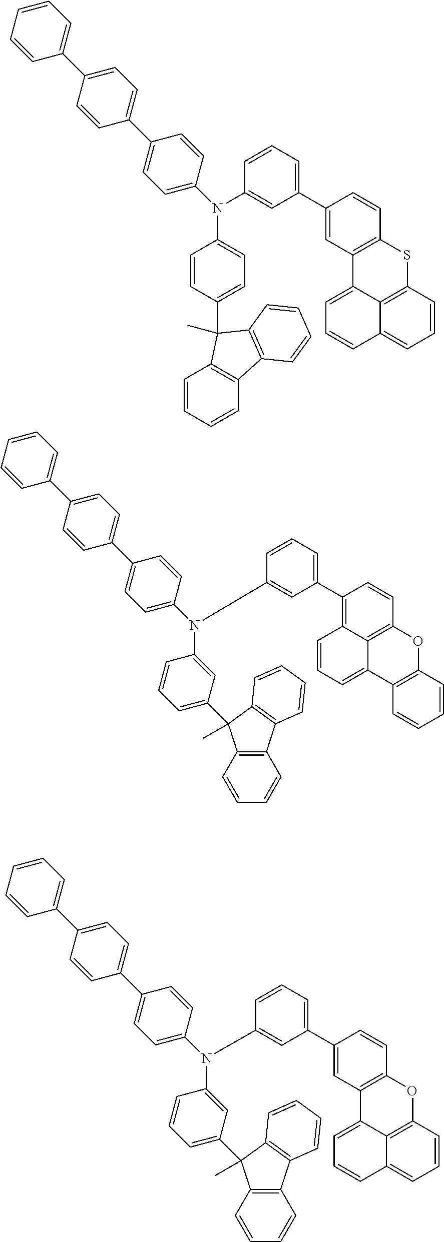 [SMILES: CC1(c2ccc(N(c3ccc(-c4ccc(-c5ccccc5)cc4)cc3)c3cccc(-c4ccc5c(c4)-c4cccc6cccc(c46)S5)c3)cc2)c2ccccc2-c2ccccc21 ; CC1(c2cccc(N(c3ccc(-c4ccc(-c5ccccc5)cc4)cc3)c3cccc(-c4ccc5c6c(cccc46)-c4ccccc4O5)c3)c2)c2ccccc2-c2ccccc21 ; CC1(c2cccc(N(c3ccc(-c4ccc(-c5ccccc5)cc4)cc3)c3cccc(-c4ccc5c(c4)-c4cccc6cccc(c46)O5)c3)c2)c2ccccc2-c2ccccc21]

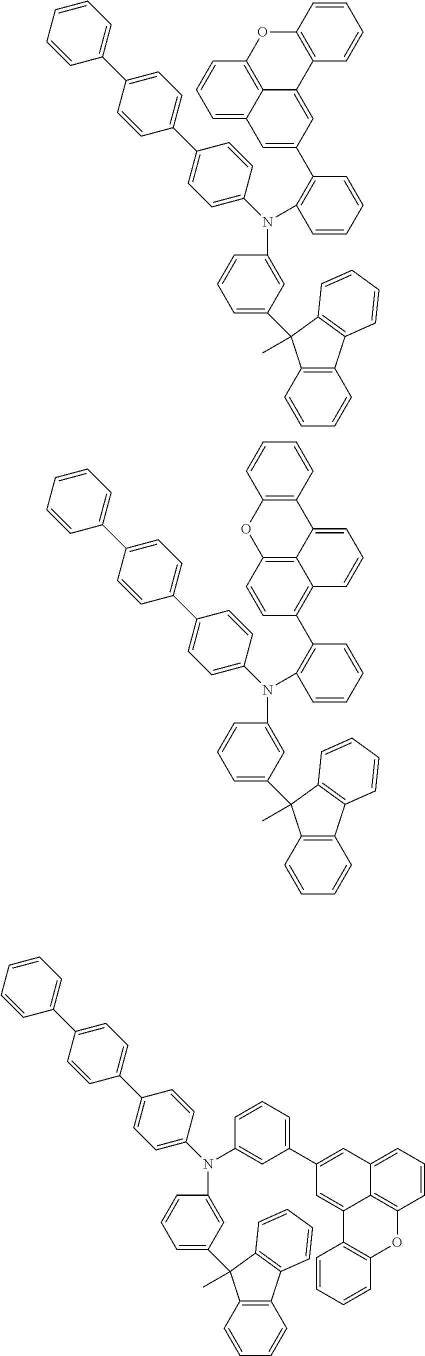

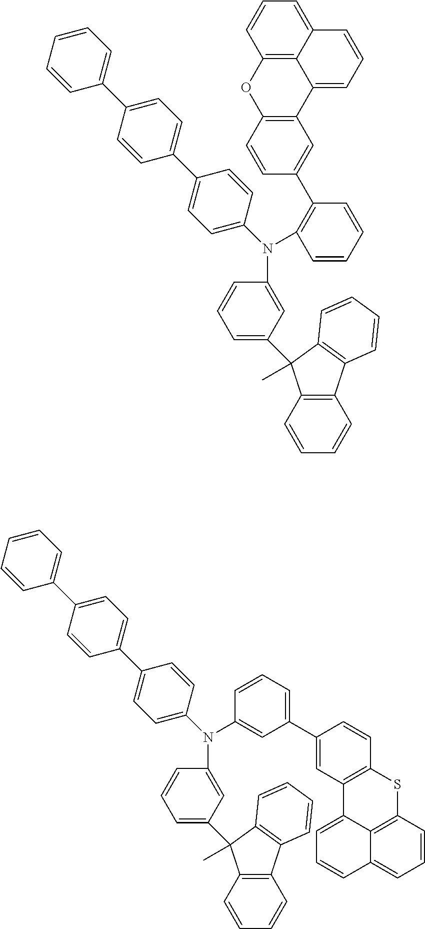 [SMILES: CC1(c2cccc(N(c3ccc(-c4ccc(-c5ccccc5)cc4)cc3)c3ccccc3-c3ccc4c(c3)-c3cccc5cccc(c35)O4)c2)c2ccccc2-c2ccccc21 ; CC1(c2cccc(N(c3ccc(-c4ccc(-c5ccccc5)cc4)cc3)c3cccc(-c4ccc5c(c4)-c4cccc6cccc(c46)S5)c3)c2)c2ccccc2-c2ccccc21]

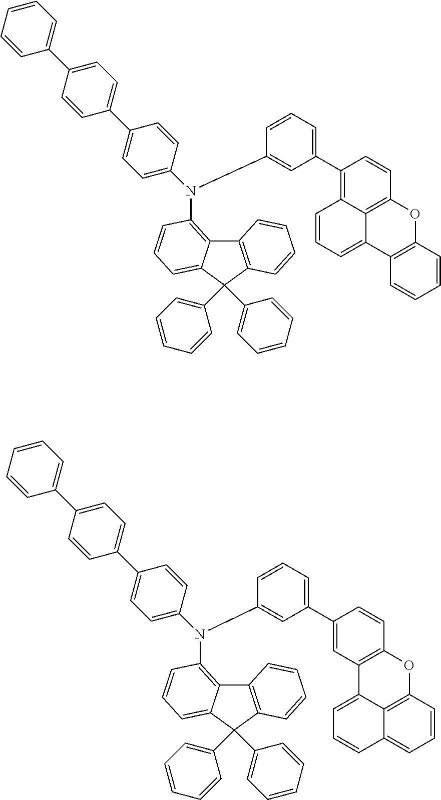 [SMILES: c1ccc(-c2ccc(-c3ccc(N(c4cccc(-c5ccc6c7c(cccc57)-c5ccccc5O6)c4)c4cccc5c4-c4ccccc4C5(c4ccccc4)c4ccccc4)cc3)cc2)cc1 ; c1ccc(-c2ccc(-c3ccc(N(c4cccc(-c5ccc6c(c5)-c5cccc7cccc(c57)O6)c4)c4cccc5c4-c4ccccc4C5(c4ccccc4)c4ccccc4)cc3)cc2)cc1]

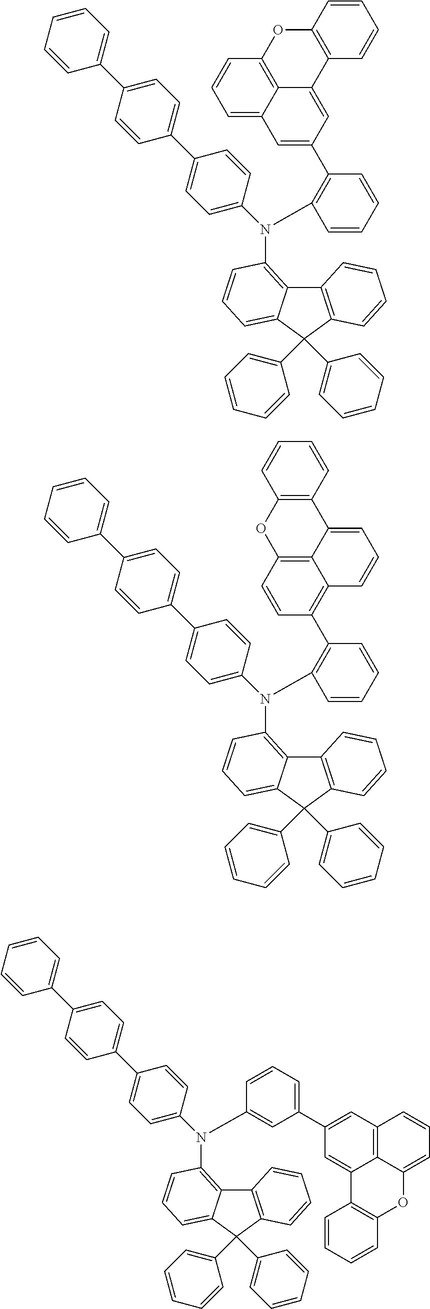

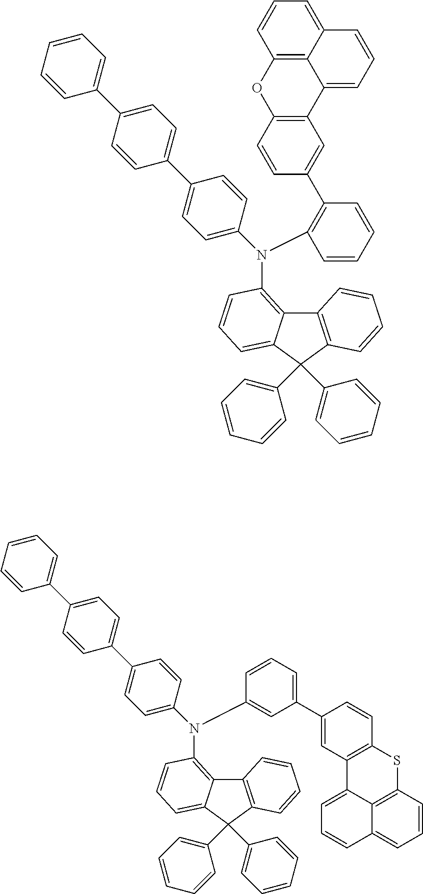 [SMILES: c1ccc(-c2ccc(-c3ccc(N(c4ccccc4-c4ccc5c(c4)-c4cccc6cccc(c46)O5)c4cccc5c4-c4ccccc4C5(c4ccccc4)c4ccccc4)cc3)cc2)cc1 ; c1ccc(-c2ccc(-c3ccc(N(c4cccc(-c5ccc6c(c5)-c5cccc7cccc(c57)S6)c4)c4cccc5c4-c4ccccc4C5(c4ccccc4)c4ccccc4)cc3)cc2)cc1]

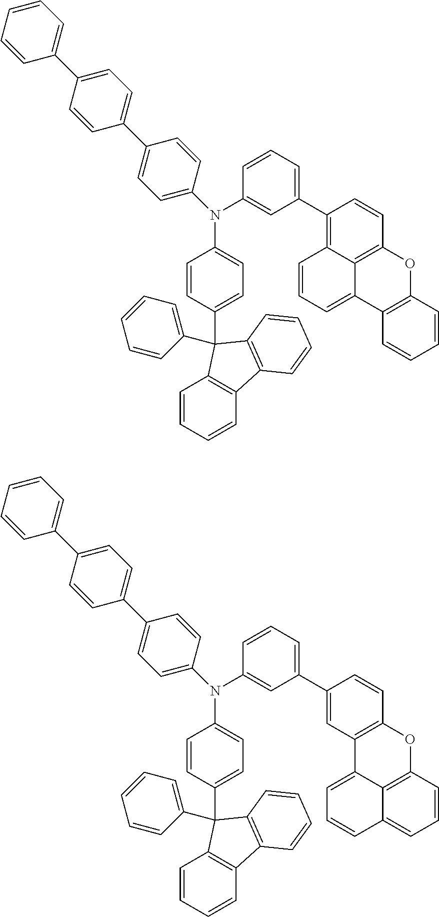 [SMILES: c1ccc(-c2ccc(-c3ccc(N(c4ccc(C5(c6ccccc6)c6ccccc6-c6ccccc65)cc4)c4cccc(-c5ccc6c7c(cccc57)-c5ccccc5O6)c4)cc3)cc2)cc1 ; c1ccc(-c2ccc(-c3ccc(N(c4ccc(C5(c6ccccc6)c6ccccc6-c6ccccc65)cc4)c4cccc(-c5ccc6c(c5)-c5cccc7cccc(c57)O6)c4)cc3)cc2)cc1]

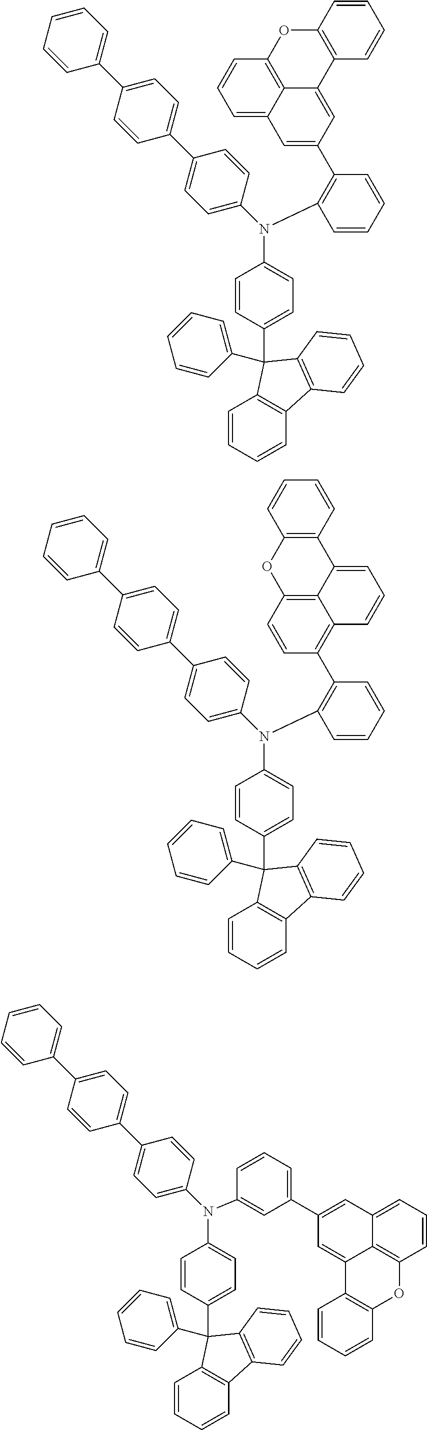

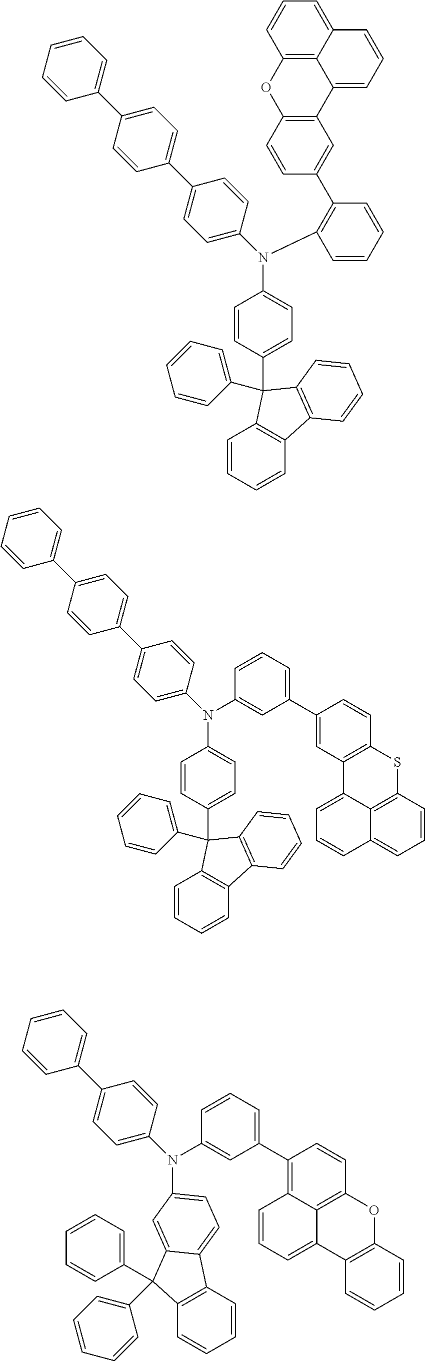 [SMILES: c1ccc(-c2ccc(-c3ccc(N(c4ccc(C5(c6ccccc6)c6ccccc6-c6ccccc65)cc4)c4ccccc4-c4ccc5c(c4)-c4cccc6cccc(c46)O5)cc3)cc2)cc1 ; c1ccc(-c2ccc(-c3ccc(N(c4ccc(C5(c6ccccc6)c6ccccc6-c6ccccc65)cc4)c4cccc(-c5ccc6c(c5)-c5cccc7cccc(c57)S6)c4)cc3)cc2)cc1 ; c1ccc(-c2ccc(N(c3cccc(-c4ccc5c6c(cccc46)-c4ccccc4O5)c3)c3ccc4c(c3)C(c3ccccc3)(c3ccccc3)c3ccccc3-4)cc2)cc1]

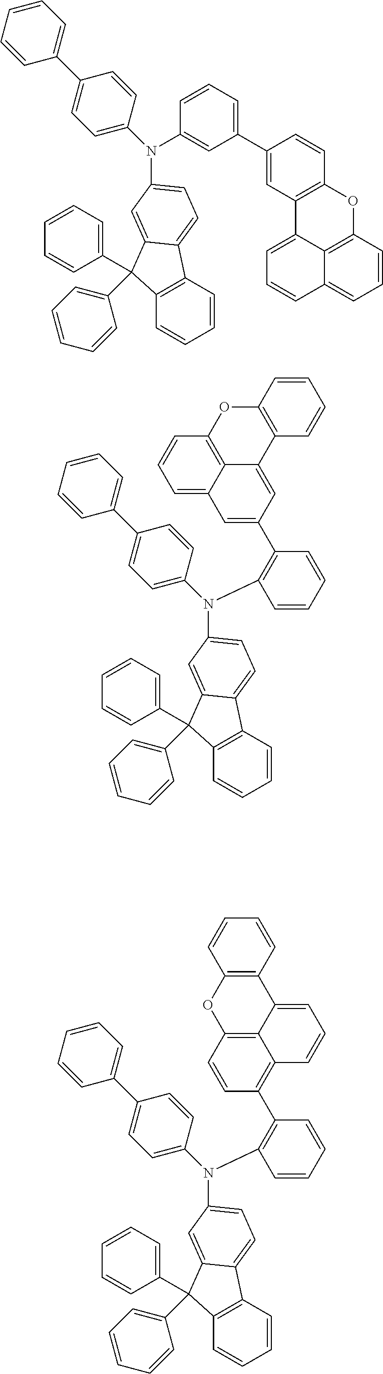 [SMILES: c1ccc(-c2ccc(N(c3cccc(-c4ccc5c(c4)-c4cccc6cccc(c46)O5)c3)c3ccc4c(c3)C(c3ccccc3)(c3ccccc3)c3ccccc3-4)cc2)cc1 ; c1ccc(-c2ccc(N(c3ccc4c(c3)C(c3ccccc3)(c3ccccc3)c3ccccc3-4)c3ccccc3-c3cc4c5c(cccc5c3)Oc3ccccc3-4)cc2)cc1 ; c1ccc(-c2ccc(N(c3ccc4c(c3)C(c3ccccc3)(c3ccccc3)c3ccccc3-4)c3ccccc3-c3ccc4c5c(cccc35)-c3ccccc3O4)cc2)cc1]

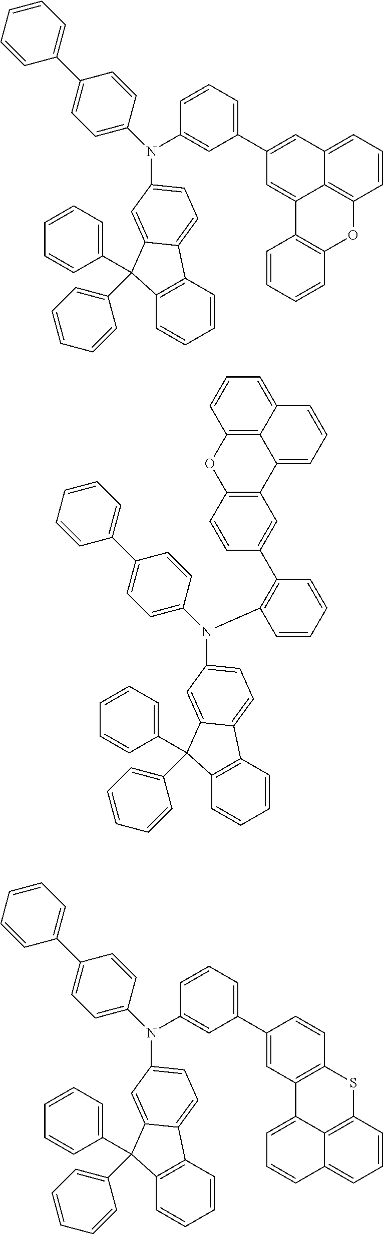 [SMILES: c1ccc(-c2ccc(N(c3cccc(-c4cc5c6c(cccc6c4)Oc4ccccc4-5)c3)c3ccc4c(c3)C(c3ccccc3)(c3ccccc3)c3ccccc3-4)cc2)cc1 ; c1ccc(-c2ccc(N(c3ccc4c(c3)C(c3ccccc3)(c3ccccc3)c3ccccc3-4)c3ccccc3-c3ccc4c(c3)-c3cccc5cccc(c35)O4)cc2)cc1 ; c1ccc(-c2ccc(N(c3cccc(-c4ccc5c(c4)-c4cccc6cccc(c46)S5)c3)c3ccc4c(c3)C(c3ccccc3)(c3ccccc3)c3ccccc3-4)cc2)cc1]

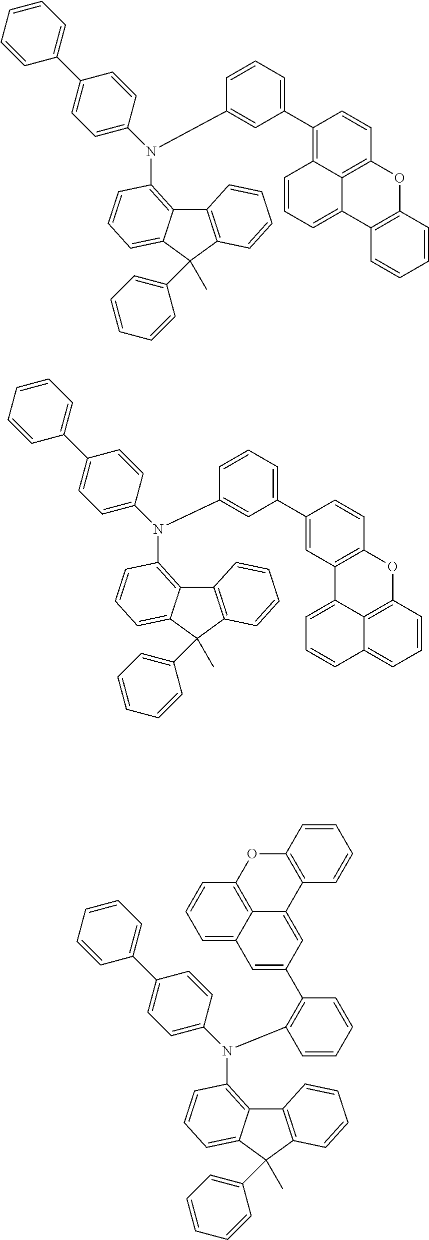 [SMILES: CC1(c2ccccc2)c2ccccc2-c2c(N(c3ccc(-c4ccccc4)cc3)c3cccc(-c4ccc5c6c(cccc46)-c4ccccc4O5)c3)cccc21 ; CC1(c2ccccc2)c2ccccc2-c2c(N(c3ccc(-c4ccccc4)cc3)c3cccc(-c4ccc5c(c4)-c4cccc6cccc(c46)O5)c3)cccc21 ; CC1(c2ccccc2)c2ccccc2-c2c(N(c3ccc(-c4ccccc4)cc3)c3ccccc3-c3cc4c5c(cccc5c3)Oc3ccccc3-4)cccc21]

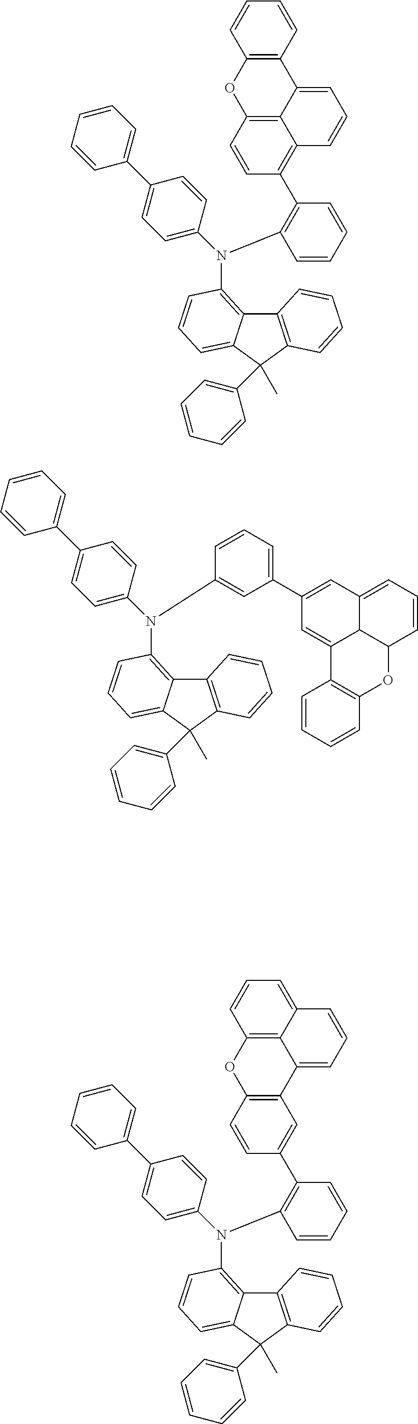 [SMILES: CC1(c2ccccc2)c2ccccc2-c2c(N(c3ccc(-c4ccccc4)cc3)c3ccccc3-c3ccc4c5c(cccc35)-c3ccccc3O4)cccc21 ; CC1(c2ccccc2)c2ccccc2-c2c(N(c3ccc(-c4ccccc4)cc3)c3cccc(C4=CC5=CC=CC6Oc7ccccc7C(=C4)C56)c3)cccc21 ; CC1(c2ccccc2)c2ccccc2-c2c(N(c3ccc(-c4ccccc4)cc3)c3ccccc3-c3ccc4c(c3)-c3cccc5cccc(c35)O4)cccc21]

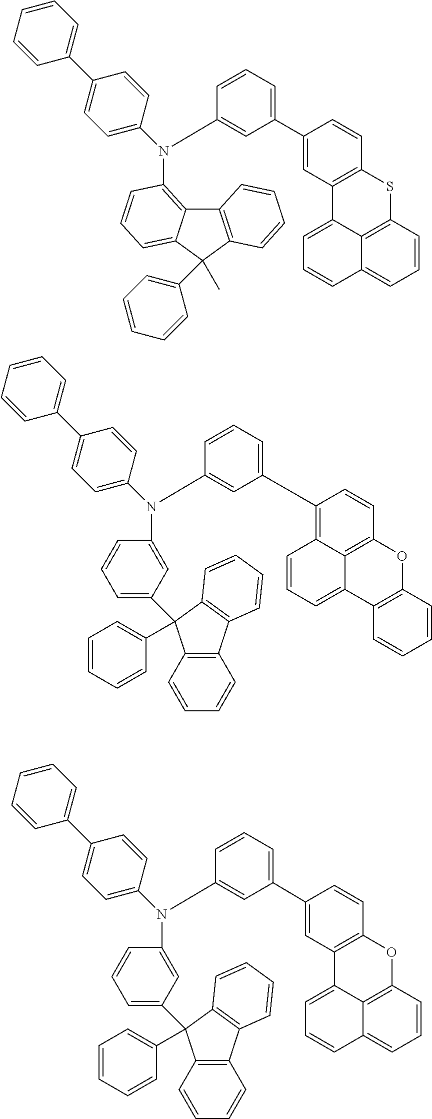 [SMILES: CC1(c2ccccc2)c2ccccc2-c2c(N(c3ccc(-c4ccccc4)cc3)c3cccc(-c4ccc5c(c4)-c4cccc6cccc(c46)S5)c3)cccc21 ; c1ccc(-c2ccc(N(c3cccc(-c4ccc5c6c(cccc46)-c4ccccc4O5)c3)c3cccc(C4(c5ccccc5)c5ccccc5-c5ccccc54)c3)cc2)cc1 ; c1ccc(-c2ccc(N(c3cccc(-c4ccc5c(c4)-c4cccc6cccc(c46)O5)c3)c3cccc(C4(c5ccccc5)c5ccccc5-c5ccccc54)c3)cc2)cc1]

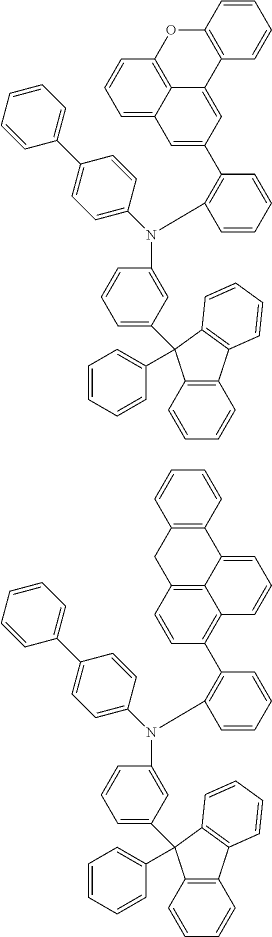

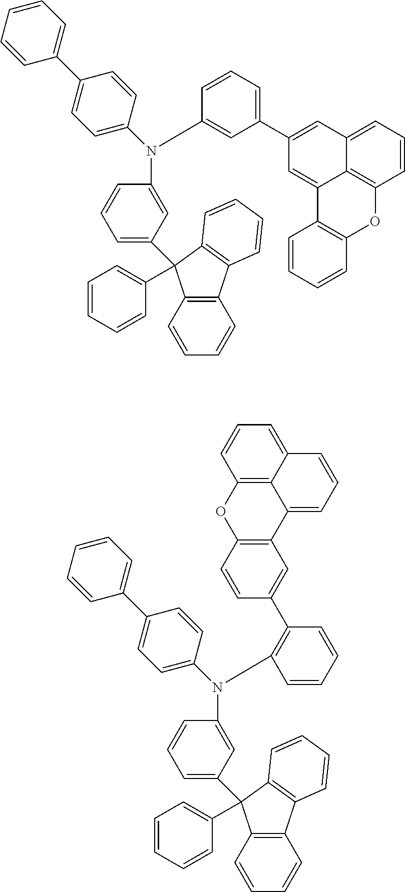 [SMILES: c1ccc(-c2ccc(N(c3cccc(-c4cc5c6c(cccc6c4)Oc4ccccc4-5)c3)c3cccc(C4(c5ccccc5)c5ccccc5-c5ccccc54)c3)cc2)cc1 ; c1ccc(-c2ccc(N(c3cccc(C4(c5ccccc5)c5ccccc5-c5ccccc54)c3)c3ccccc3-c3ccc4c(c3)-c3cccc5cccc(c35)O4)cc2)cc1]

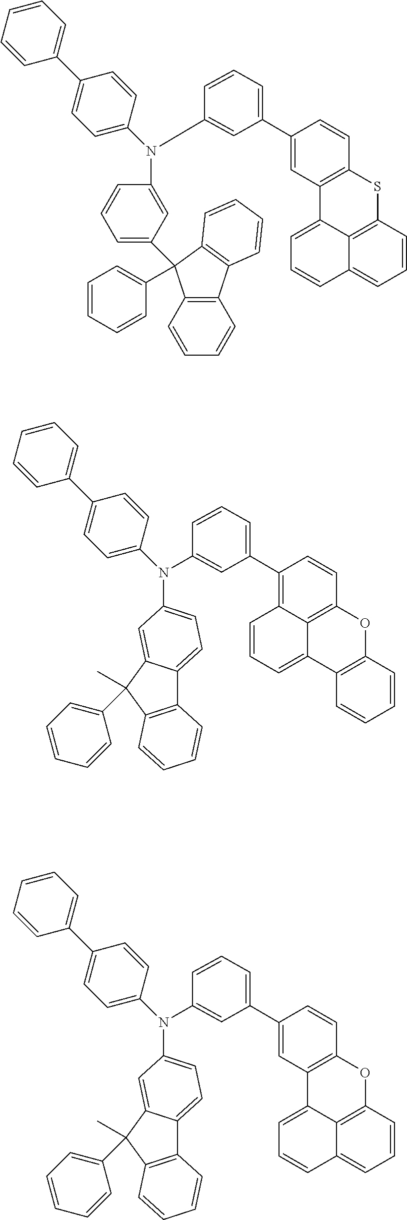 [SMILES: c1ccc(-c2ccc(N(c3cccc(-c4ccc5c(c4)-c4cccc6cccc(c46)S5)c3)c3cccc(C4(c5ccccc5)c5ccccc5-c5ccccc54)c3)cc2)cc1 ; CC1(c2ccccc2)c2ccccc2-c2ccc(N(c3ccc(-c4ccccc4)cc3)c3cccc(-c4ccc5c6c(cccc46)-c4ccccc4O5)c3)cc21 ; CC1(c2ccccc2)c2ccccc2-c2ccc(N(c3ccc(-c4ccccc4)cc3)c3cccc(-c4ccc5c(c4)-c4cccc6cccc(c46)O5)c3)cc21]

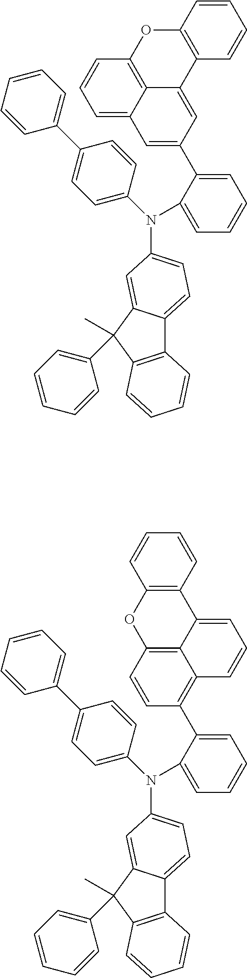 [SMILES: CC1(c2ccccc2)c2ccccc2-c2ccc(N(c3ccc(-c4ccccc4)cc3)c3ccccc3-c3cc4c5c(cccc5c3)Oc3ccccc3-4)cc21 ; CC1(c2ccccc2)c2ccccc2-c2ccc(N(c3ccc(-c4ccccc4)cc3)c3ccccc3-c3ccc4c5c(cccc35)-c3ccccc3O4)cc21]

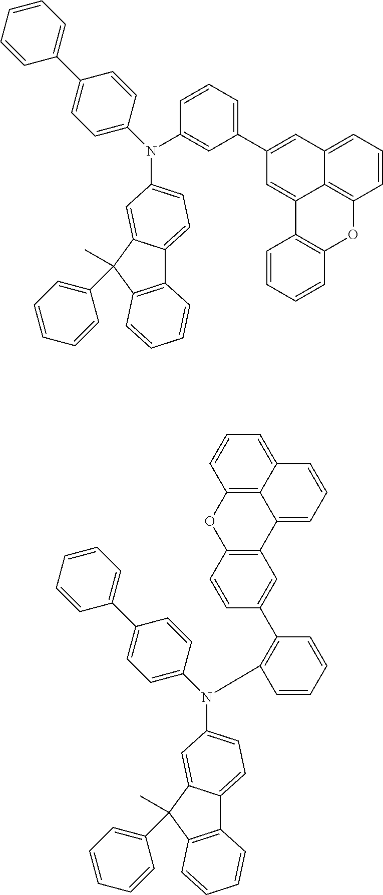 [SMILES: CC1(c2ccccc2)c2ccccc2-c2ccc(N(c3ccc(-c4ccccc4)cc3)c3cccc(-c4cc5c6c(cccc6c4)Oc4ccccc4-5)c3)cc21 ; CC1(c2ccccc2)c2ccccc2-c2ccc(N(c3ccc(-c4ccccc4)cc3)c3ccccc3-c3ccc4c(c3)-c3cccc5cccc(c35)O4)cc21]

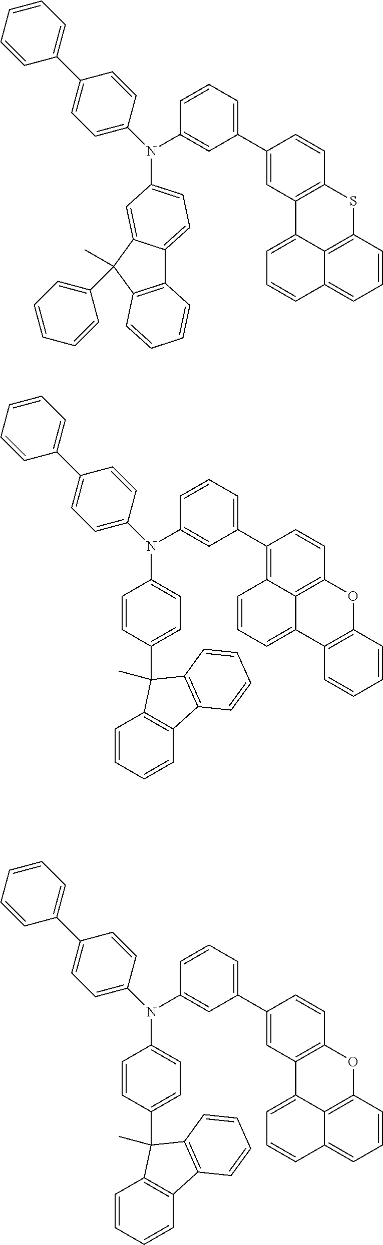 [SMILES: CC1(c2ccccc2)c2ccccc2-c2ccc(N(c3ccc(-c4ccccc4)cc3)c3cccc(-c4ccc5c(c4)-c4cccc6cccc(c46)S5)c3)cc21 ; CC1(c2ccc(N(c3ccc(-c4ccccc4)cc3)c3cccc(-c4ccc5c6c(cccc46)-c4ccccc4O5)c3)cc2)c2ccccc2-c2ccccc21 ; CC1(c2ccc(N(c3ccc(-c4ccccc4)cc3)c3cccc(-c4ccc5c(c4)-c4cccc6cccc(c46)O5)c3)cc2)c2ccccc2-c2ccccc21]

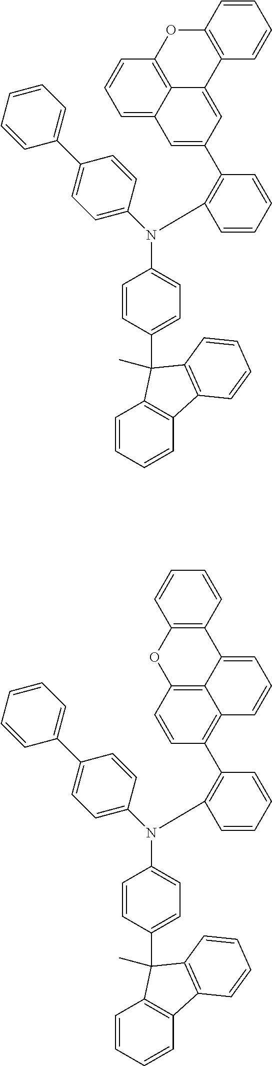 [SMILES: CC1(c2ccc(N(c3ccc(-c4ccccc4)cc3)c3ccccc3-c3cc4c5c(cccc5c3)Oc3ccccc3-4)cc2)c2ccccc2-c2ccccc21 ; CC1(c2ccc(N(c3ccc(-c4ccccc4)cc3)c3ccccc3-c3ccc4c5c(cccc35)-c3ccccc3O4)cc2)c2ccccc2-c2ccccc21]

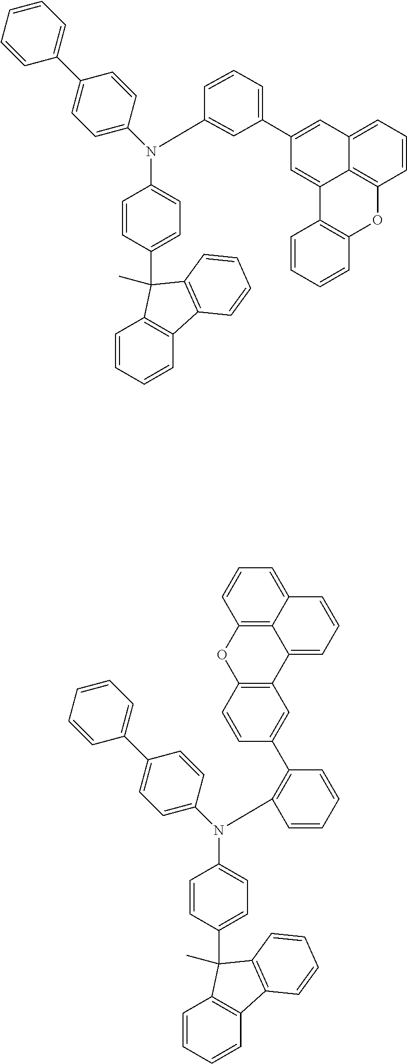 [SMILES: CC1(c2ccc(N(c3ccc(-c4ccccc4)cc3)c3cccc(-c4cc5c6c(cccc6c4)Oc4ccccc4-5)c3)cc2)c2ccccc2-c2ccccc21 ; CC1(c2ccc(N(c3ccc(-c4ccccc4)cc3)c3ccccc3-c3ccc4c(c3)-c3cccc5cccc(c35)O4)cc2)c2ccccc2-c2ccccc21]

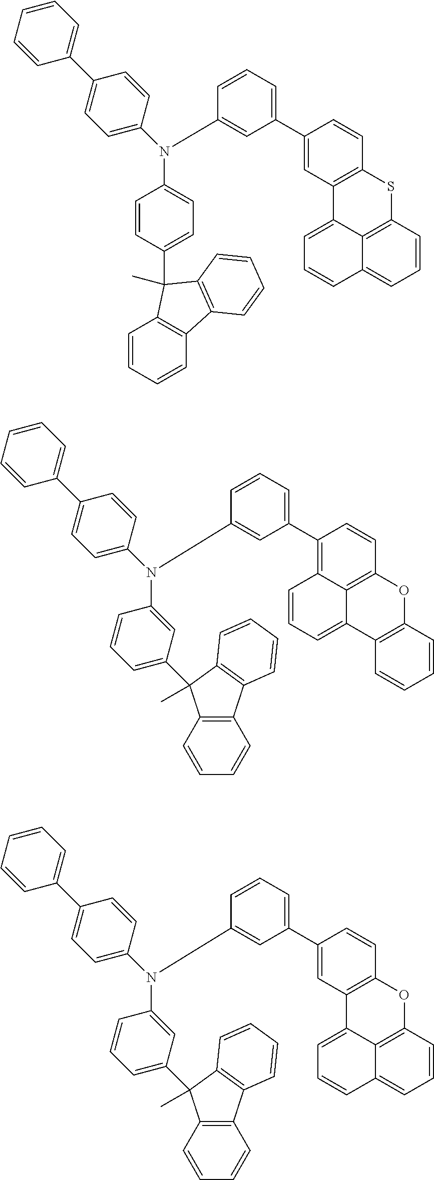 [SMILES: CC1(c2ccc(N(c3ccc(-c4ccccc4)cc3)c3cccc(-c4ccc5c(c4)-c4cccc6cccc(c46)S5)c3)cc2)c2ccccc2-c2ccccc21 ; CC1(c2cccc(N(c3ccc(-c4ccccc4)cc3)c3cccc(-c4ccc5c6c(cccc46)-c4ccccc4O5)c3)c2)c2ccccc2-c2ccccc21 ; CC1(c2cccc(N(c3ccc(-c4ccccc4)cc3)c3cccc(-c4ccc5c(c4)-c4cccc6cccc(c46)O5)c3)c2)c2ccccc2-c2ccccc21]

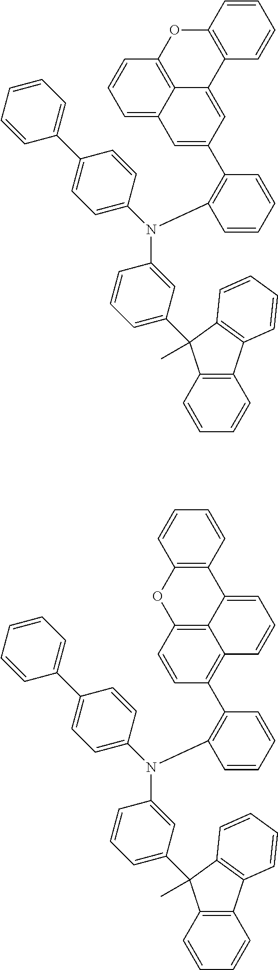 [SMILES: CC1(c2cccc(N(c3ccc(-c4ccccc4)cc3)c3ccccc3-c3cc4c5c(cccc5c3)Oc3ccccc3-4)c2)c2ccccc2-c2ccccc21 ; CC1(c2cccc(N(c3ccc(-c4ccccc4)cc3)c3ccccc3-c3ccc4c5c(cccc35)-c3ccccc3O4)c2)c2ccccc2-c2ccccc21]

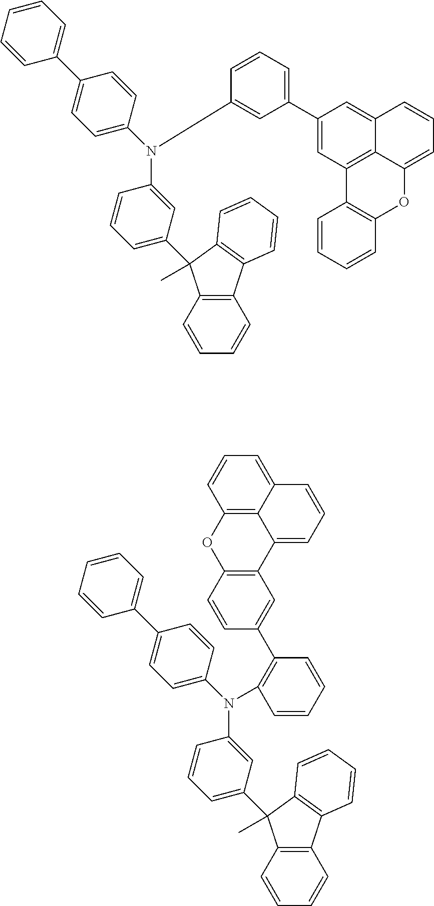 [SMILES: CC1(c2cccc(N(c3ccc(-c4ccccc4)cc3)c3cccc(-c4cc5c6c(cccc6c4)Oc4ccccc4-5)c3)c2)c2ccccc2-c2ccccc21 ; CC1(c2cccc(N(c3ccc(-c4ccccc4)cc3)c3ccccc3-c3ccc4c(c3)-c3cccc5cccc(c35)O4)c2)c2ccccc2-c2ccccc21]

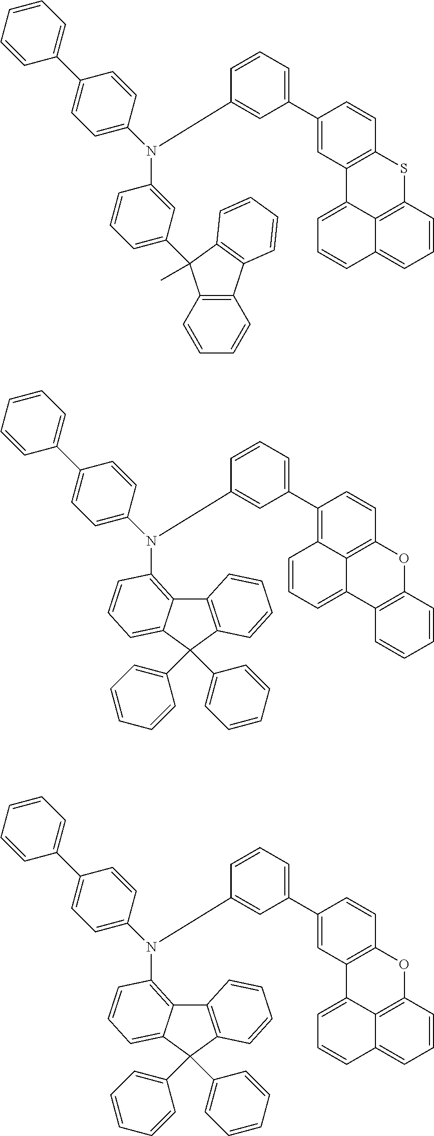 [SMILES: CC1(c2cccc(N(c3ccc(-c4ccccc4)cc3)c3cccc(-c4ccc5c(c4)-c4cccc6cccc(c46)S5)c3)c2)c2ccccc2-c2ccccc21 ; c1ccc(-c2ccc(N(c3cccc(-c4ccc5c6c(cccc46)-c4ccccc4O5)c3)c3cccc4c3-c3ccccc3C4(c3ccccc3)c3ccccc3)cc2)cc1 ; c1ccc(-c2ccc(N(c3cccc(-c4ccc5c(c4)-c4cccc6cccc(c46)O5)c3)c3cccc4c3-c3ccccc3C4(c3ccccc3)c3ccccc3)cc2)cc1]

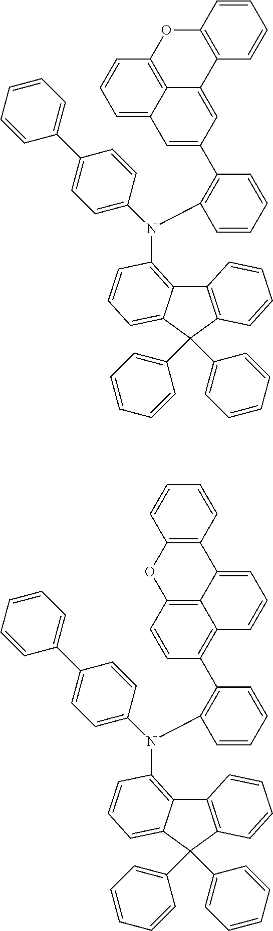 [SMILES: c1ccc(-c2ccc(N(c3ccccc3-c3cc4c5c(cccc5c3)Oc3ccccc3-4)c3cccc4c3-c3ccccc3C4(c3ccccc3)c3ccccc3)cc2)cc1 ; c1ccc(-c2ccc(N(c3ccccc3-c3ccc4c5c(cccc35)-c3ccccc3O4)c3cccc4c3-c3ccccc3C4(c3ccccc3)c3ccccc3)cc2)cc1]

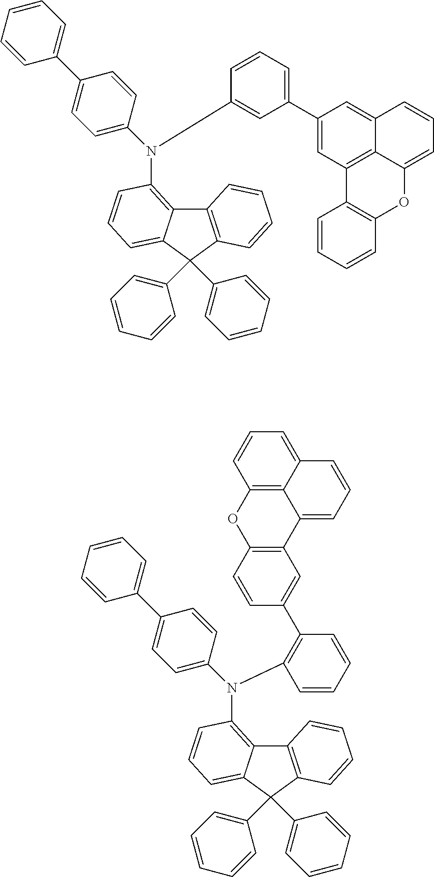 [SMILES: c1ccc(-c2ccc(N(c3cccc(-c4cc5c6c(cccc6c4)Oc4ccccc4-5)c3)c3cccc4c3-c3ccccc3C4(c3ccccc3)c3ccccc3)cc2)cc1 ; c1ccc(-c2ccc(N(c3ccccc3-c3ccc4c(c3)-c3cccc5cccc(c35)O4)c3cccc4c3-c3ccccc3C4(c3ccccc3)c3ccccc3)cc2)cc1]

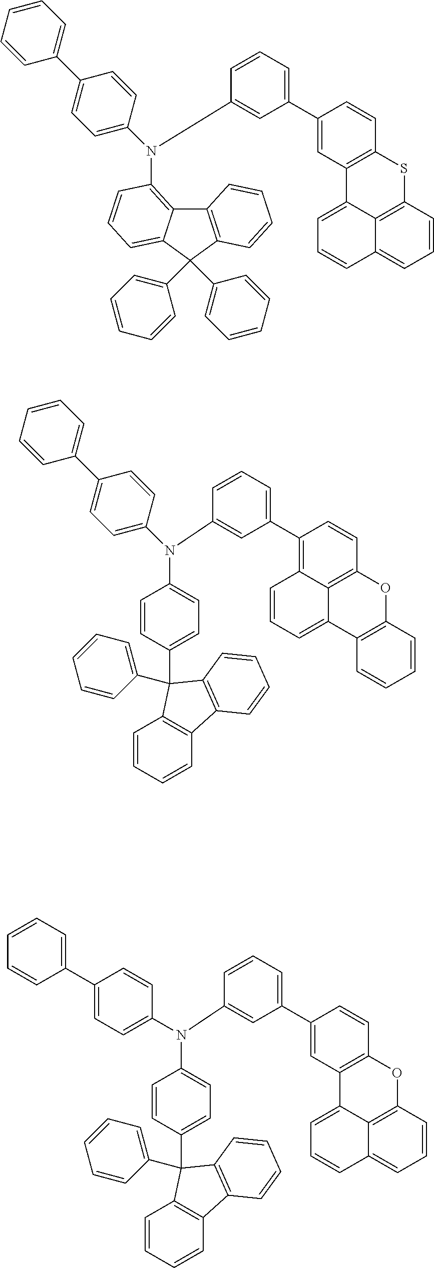 [SMILES: c1ccc(-c2ccc(N(c3cccc(-c4ccc5c(c4)-c4cccc6cccc(c46)S5)c3)c3cccc4c3-c3ccccc3C4(c3ccccc3)c3ccccc3)cc2)cc1 ; c1ccc(-c2ccc(N(c3ccc(C4(c5ccccc5)c5ccccc5-c5ccccc54)cc3)c3cccc(-c4ccc5c6c(cccc46)-c4ccccc4O5)c3)cc2)cc1 ; c1ccc(-c2ccc(N(c3ccc(C4(c5ccccc5)c5ccccc5-c5ccccc54)cc3)c3cccc(-c4ccc5c(c4)-c4cccc6cccc(c46)O5)c3)cc2)cc1]

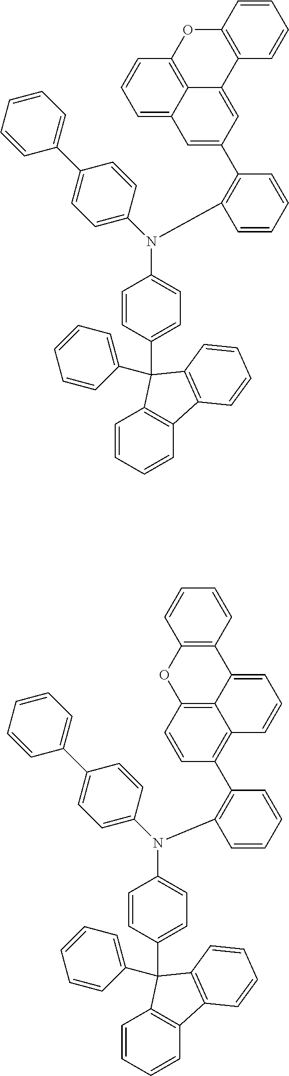 [SMILES: c1ccc(-c2ccc(N(c3ccc(C4(c5ccccc5)c5ccccc5-c5ccccc54)cc3)c3ccccc3-c3cc4c5c(cccc5c3)Oc3ccccc3-4)cc2)cc1 ; c1ccc(-c2ccc(N(c3ccc(C4(c5ccccc5)c5ccccc5-c5ccccc54)cc3)c3ccccc3-c3ccc4c5c(cccc35)-c3ccccc3O4)cc2)cc1]

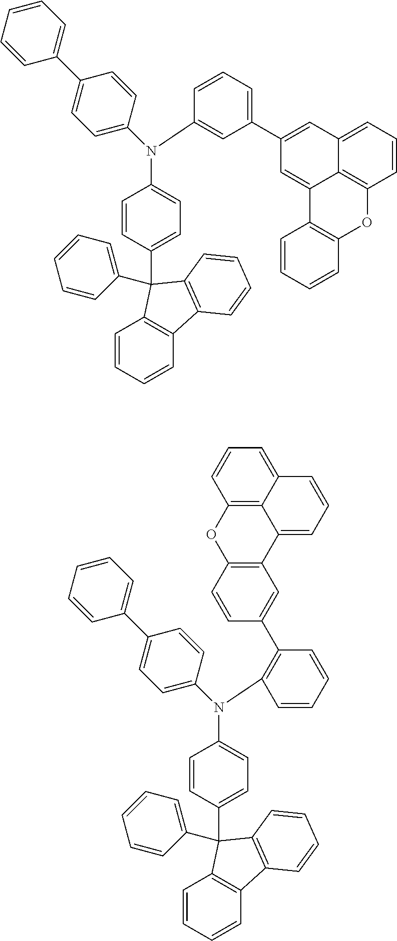 [SMILES: c1ccc(-c2ccc(N(c3ccc(C4(c5ccccc5)c5ccccc5-c5ccccc54)cc3)c3cccc(-c4cc5c6c(cccc6c4)Oc4ccccc4-5)c3)cc2)cc1 ; c1ccc(-c2ccc(N(c3ccc(C4(c5ccccc5)c5ccccc5-c5ccccc54)cc3)c3ccccc3-c3ccc4c(c3)-c3cccc5cccc(c35)O4)cc2)cc1]

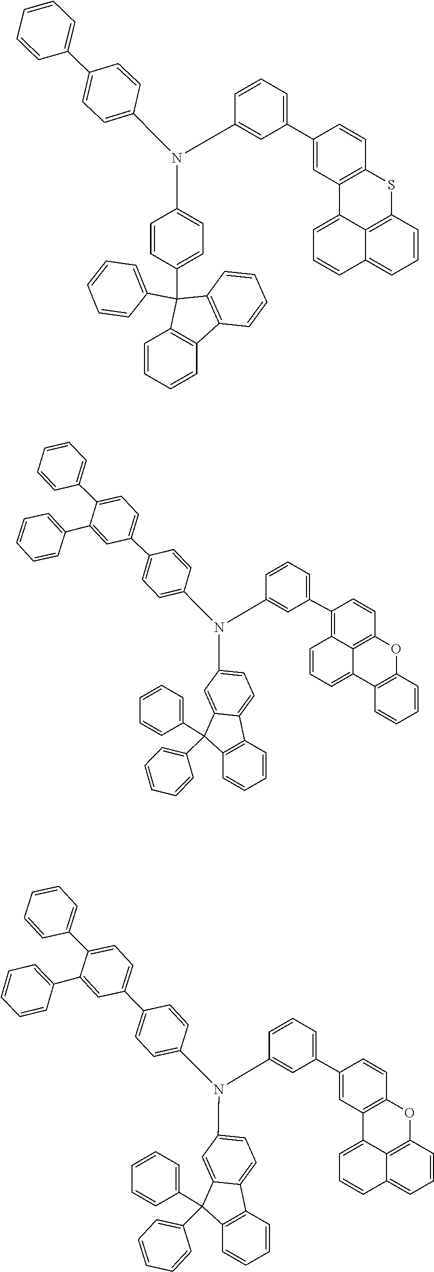 [SMILES: c1ccc(-c2ccc(N(c3ccc(C4(c5ccccc5)c5ccccc5-c5ccccc54)cc3)c3cccc(-c4ccc5c(c4)-c4cccc6cccc(c46)S5)c3)cc2)cc1 ; c1ccc(-c2ccc(-c3ccc(N(c4cccc(-c5ccc6c7c(cccc57)-c5ccccc5O6)c4)c4ccc5c(c4)C(c4ccccc4)(c4ccccc4)c4ccccc4-5)cc3)cc2-c2ccccc2)cc1 ; c1ccc(-c2ccc(-c3ccc(N(c4cccc(-c5ccc6c(c5)-c5cccc7cccc(c57)O6)c4)c4ccc5c(c4)C(c4ccccc4)(c4ccccc4)c4ccccc4-5)cc3)cc2-c2ccccc2)cc1]

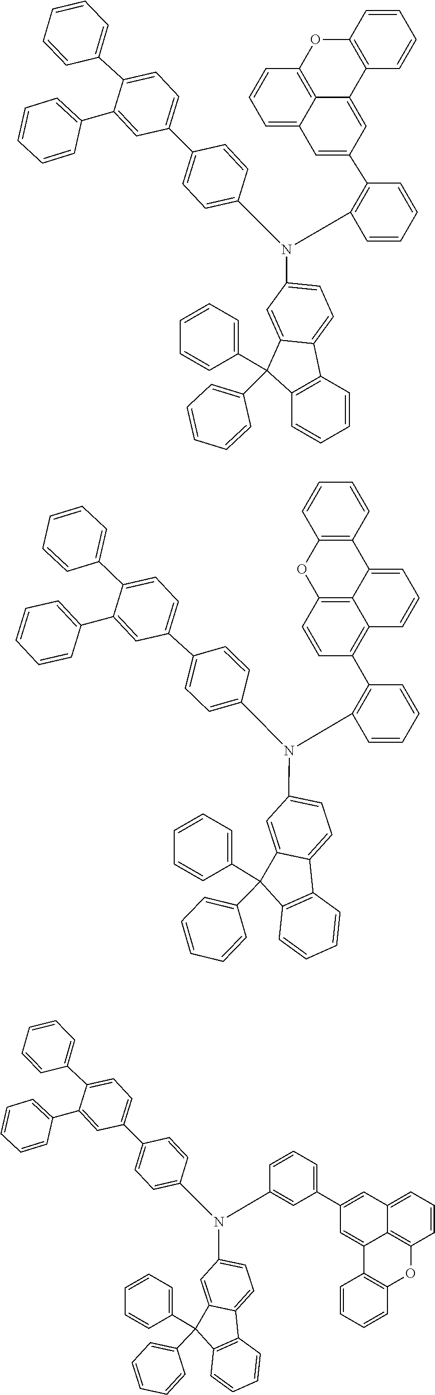

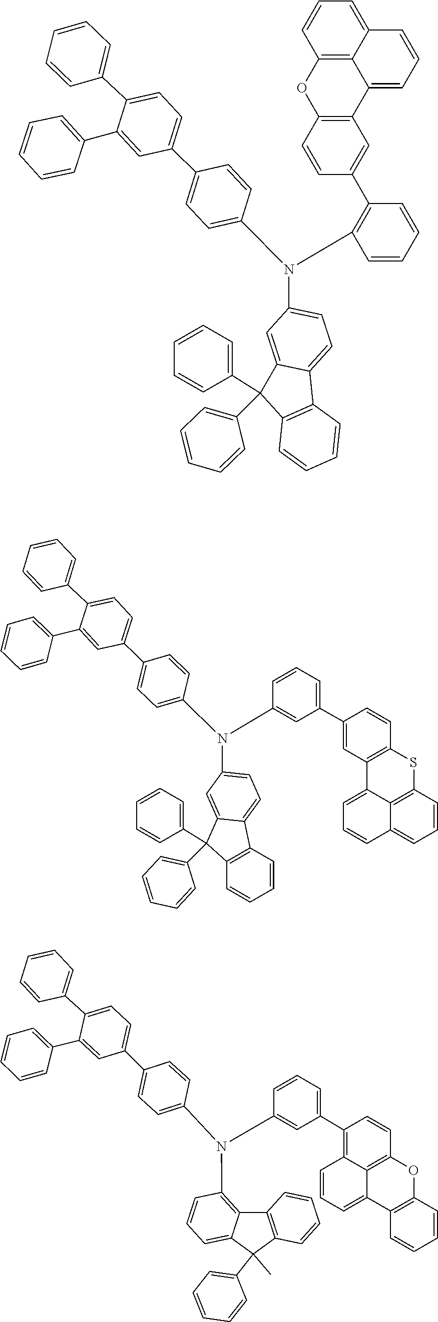 [SMILES: c1ccc(-c2ccc(-c3ccc(N(c4ccc5c(c4)C(c4ccccc4)(c4ccccc4)c4ccccc4-5)c4ccccc4-c4ccc5c(c4)-c4cccc6cccc(c46)O5)cc3)cc2-c2ccccc2)cc1 ; c1ccc(-c2ccc(-c3ccc(N(c4cccc(-c5ccc6c(c5)-c5cccc7cccc(c57)S6)c4)c4ccc5c(c4)C(c4ccccc4)(c4ccccc4)c4ccccc4-5)cc3)cc2-c2ccccc2)cc1 ; CC1(c2ccccc2)c2ccccc2-c2c(N(c3ccc(-c4ccc(-c5ccccc5)c(-c5ccccc5)c4)cc3)c3cccc(-c4ccc5c6c(cccc46)-c4ccccc4O5)c3)cccc21]

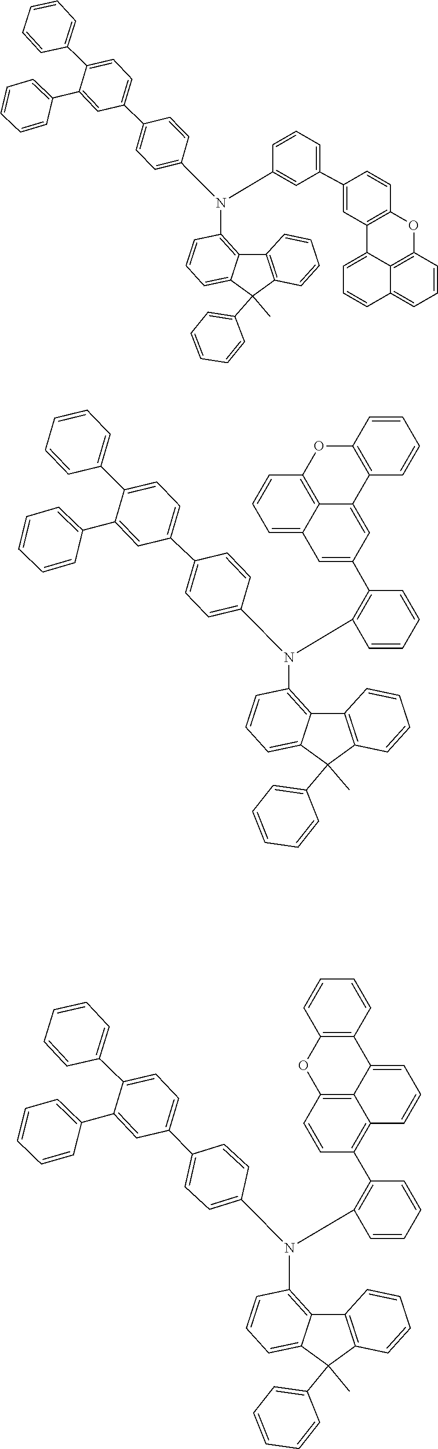 [SMILES: CC1(c2ccccc2)c2ccccc2-c2c(N(c3ccc(-c4ccc(-c5ccccc5)c(-c5ccccc5)c4)cc3)c3cccc(-c4ccc5c(c4)-c4cccc6cccc(c46)O5)c3)cccc21 ; CC1(c2ccccc2)c2ccccc2-c2c(N(c3ccc(-c4ccc(-c5ccccc5)c(-c5ccccc5)c4)cc3)c3ccccc3-c3cc4c5c(cccc5c3)Oc3ccccc3-4)cccc21 ; CC1(c2ccccc2)c2ccccc2-c2c(N(c3ccc(-c4ccc(-c5ccccc5)c(-c5ccccc5)c4)cc3)c3ccccc3-c3ccc4c5c(cccc35)-c3ccccc3O4)cccc21]

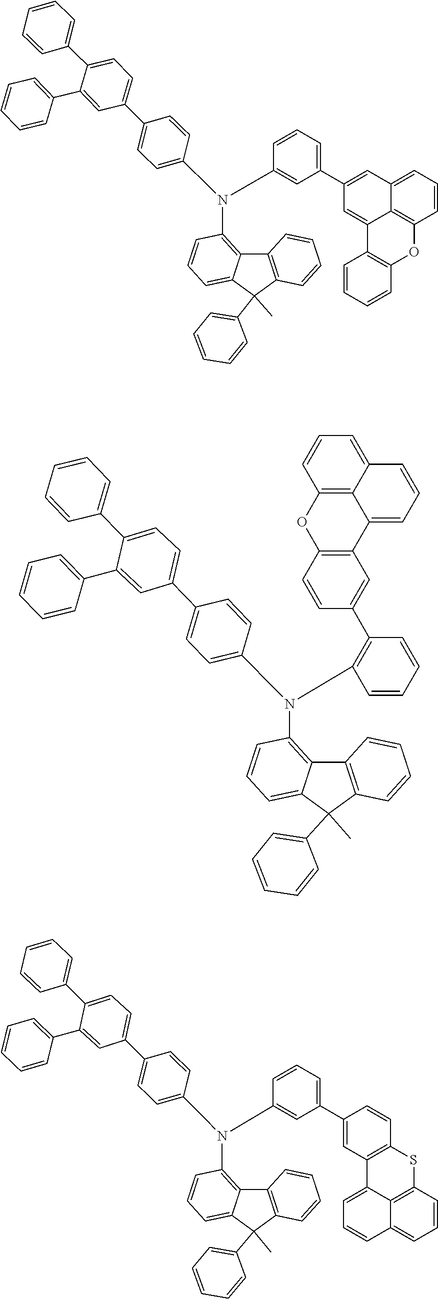 [SMILES: CC1(c2ccccc2)c2ccccc2-c2c(N(c3ccc(-c4ccc(-c5ccccc5)c(-c5ccccc5)c4)cc3)c3cccc(-c4cc5c6c(cccc6c4)Oc4ccccc4-5)c3)cccc21 ; CC1(c2ccccc2)c2ccccc2-c2c(N(c3ccc(-c4ccc(-c5ccccc5)c(-c5ccccc5)c4)cc3)c3ccccc3-c3ccc4c(c3)-c3cccc5cccc(c35)O4)cccc21 ; CC1(c2ccccc2)c2ccccc2-c2c(N(c3ccc(-c4ccc(-c5ccccc5)c(-c5ccccc5)c4)cc3)c3cccc(-c4ccc5c(c4)-c4cccc6cccc(c46)S5)c3)cccc21]

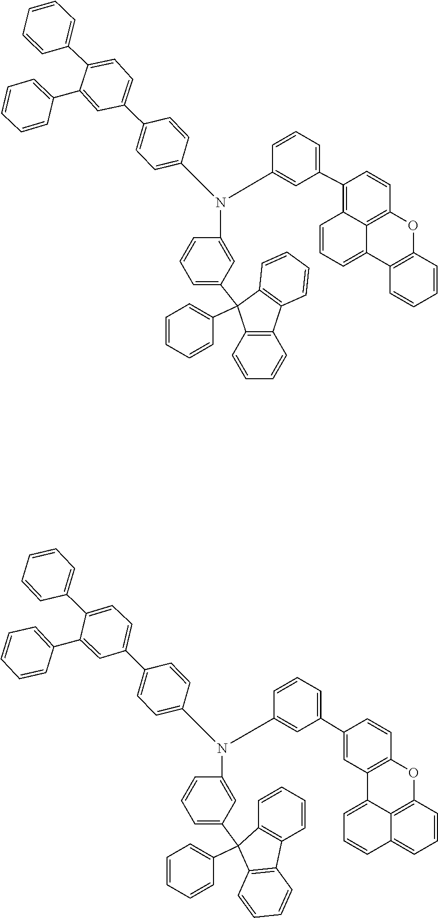 [SMILES: c1ccc(-c2ccc(-c3ccc(N(c4cccc(-c5ccc6c7c(cccc57)-c5ccccc5O6)c4)c4cccc(C5(c6ccccc6)c6ccccc6-c6ccccc65)c4)cc3)cc2-c2ccccc2)cc1 ; c1ccc(-c2ccc(-c3ccc(N(c4cccc(-c5ccc6c(c5)-c5cccc7cccc(c57)O6)c4)c4cccc(C5(c6ccccc6)c6ccccc6-c6ccccc65)c4)cc3)cc2-c2ccccc2)cc1]

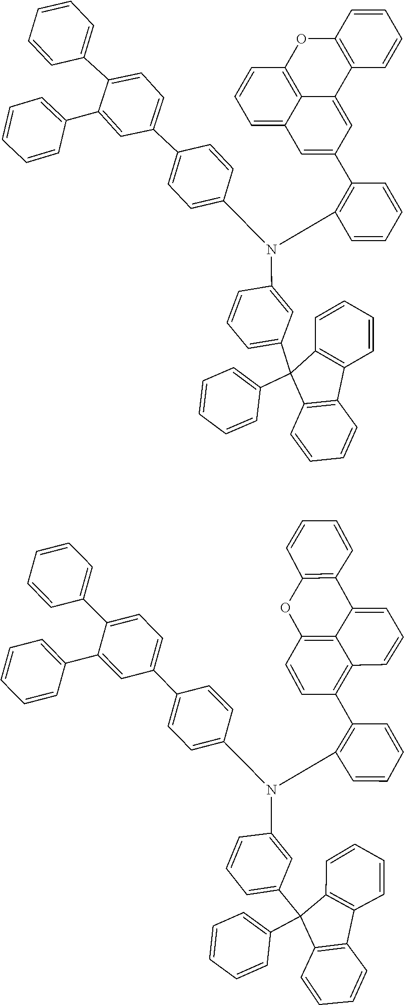 [SMILES: c1ccc(-c2ccc(-c3ccc(N(c4cccc(C5(c6ccccc6)c6ccccc6-c6ccccc65)c4)c4ccccc4-c4cc5c6c(cccc6c4)Oc4ccccc4-5)cc3)cc2-c2ccccc2)cc1 ; c1ccc(-c2ccc(-c3ccc(N(c4cccc(C5(c6ccccc6)c6ccccc6-c6ccccc65)c4)c4ccccc4-c4ccc5c6c(cccc46)-c4ccccc4O5)cc3)cc2-c2ccccc2)cc1]

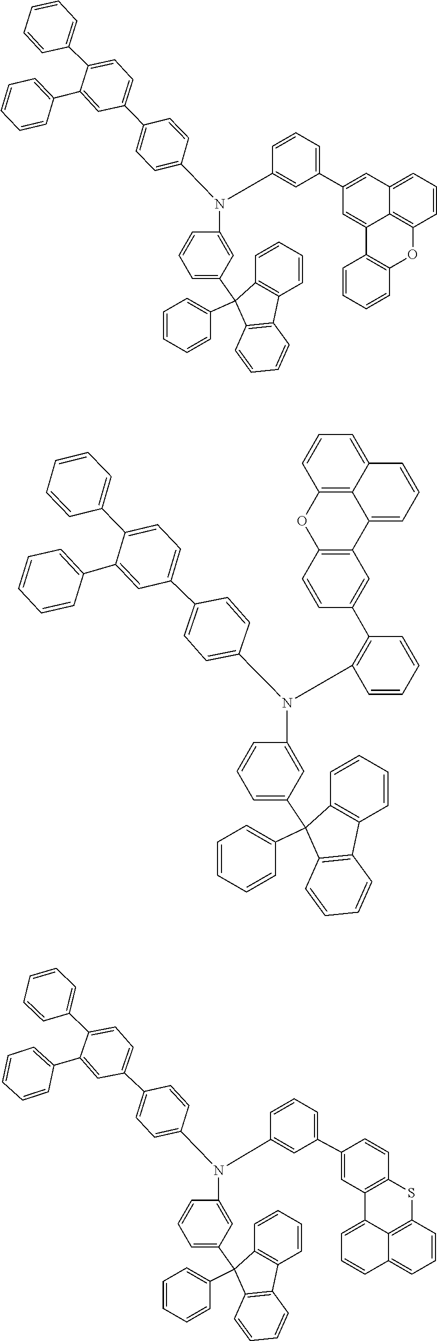 [SMILES: c1ccc(-c2ccc(-c3ccc(N(c4cccc(-c5cc6c7c(cccc7c5)Oc5ccccc5-6)c4)c4cccc(C5(c6ccccc6)c6ccccc6-c6ccccc65)c4)cc3)cc2-c2ccccc2)cc1 ; c1ccc(-c2ccc(-c3ccc(N(c4cccc(C5(c6ccccc6)c6ccccc6-c6ccccc65)c4)c4ccccc4-c4ccc5c(c4)-c4cccc6cccc(c46)O5)cc3)cc2-c2ccccc2)cc1 ; c1ccc(-c2ccc(-c3ccc(N(c4cccc(-c5ccc6c(c5)-c5cccc7cccc(c57)S6)c4)c4cccc(C5(c6ccccc6)c6ccccc6-c6ccccc65)c4)cc3)cc2-c2ccccc2)cc1]

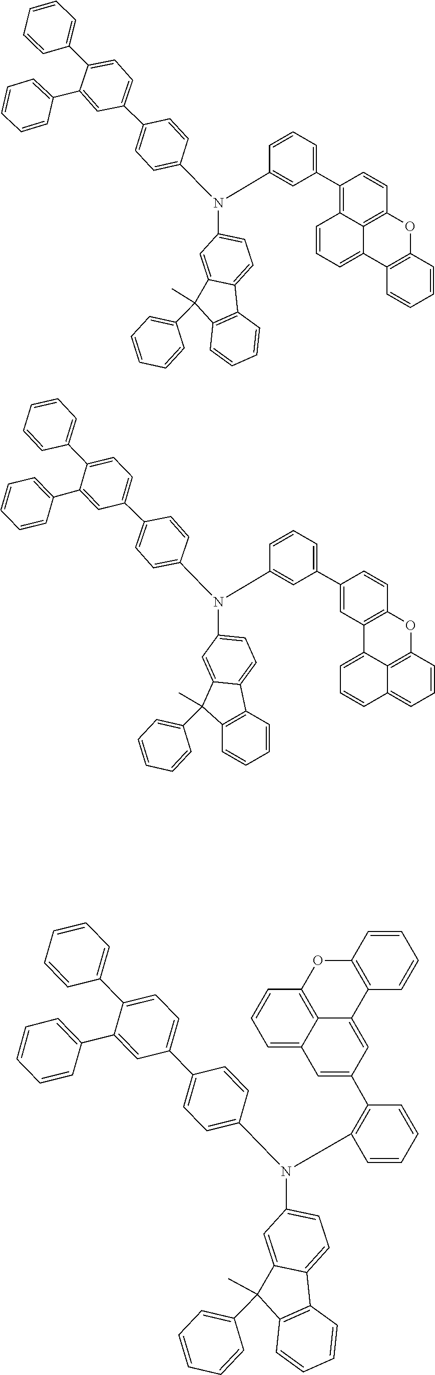 [SMILES: CC1(c2ccccc2)c2ccccc2-c2ccc(N(c3ccc(-c4ccc(-c5ccccc5)c(-c5ccccc5)c4)cc3)c3cccc(-c4ccc5c6c(cccc46)-c4ccccc4O5)c3)cc21 ; CC1(c2ccccc2)c2ccccc2-c2ccc(N(c3ccc(-c4ccc(-c5ccccc5)c(-c5ccccc5)c4)cc3)c3cccc(-c4ccc5c(c4)-c4cccc6cccc(c46)O5)c3)cc21 ; CC1(c2ccccc2)c2ccccc2-c2ccc(N(c3ccc(-c4ccc(-c5ccccc5)c(-c5ccccc5)c4)cc3)c3ccccc3-c3cc4c5c(cccc5c3)Oc3ccccc3-4)cc21]

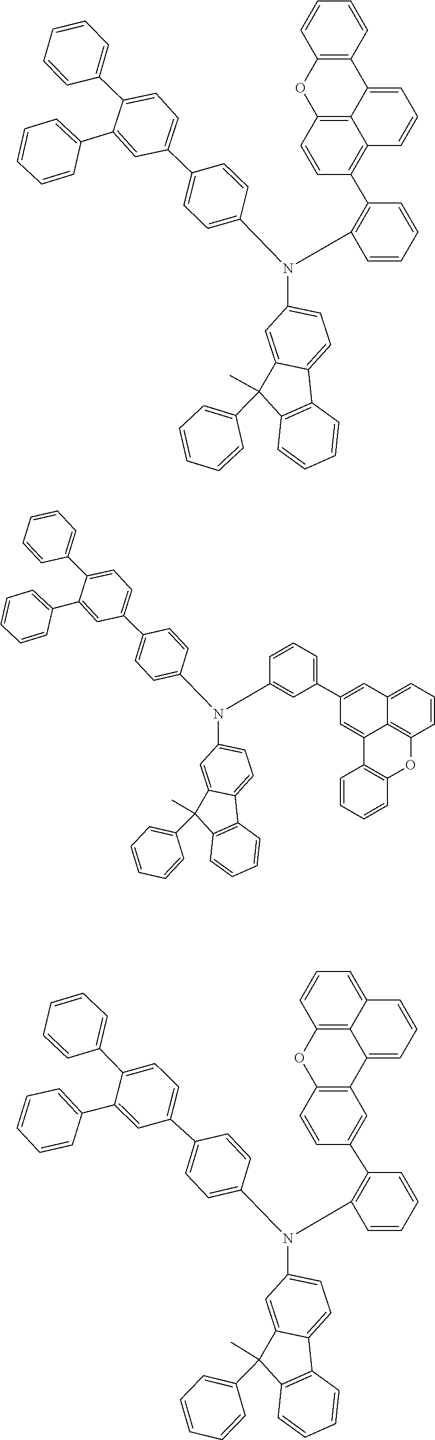 [SMILES: CC1(c2ccccc2)c2ccccc2-c2ccc(N(c3ccc(-c4ccc(-c5ccccc5)c(-c5ccccc5)c4)cc3)c3ccccc3-c3ccc4c5c(cccc35)-c3ccccc3O4)cc21 ; CC1(c2ccccc2)c2ccccc2-c2ccc(N(c3ccc(-c4ccc(-c5ccccc5)c(-c5ccccc5)c4)cc3)c3cccc(-c4cc5c6c(cccc6c4)Oc4ccccc4-5)c3)cc21 ; CC1(c2ccccc2)c2ccccc2-c2ccc(N(c3ccc(-c4ccc(-c5ccccc5)c(-c5ccccc5)c4)cc3)c3ccccc3-c3ccc4c(c3)-c3cccc5cccc(c35)O4)cc21]

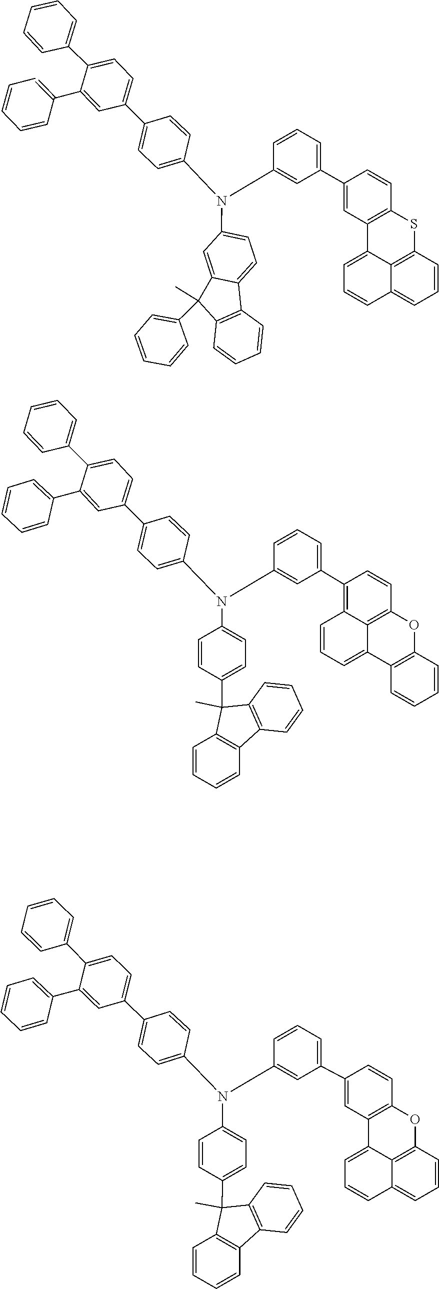 [SMILES: C=C(/C=C\C=C/C)c1cc(-c2ccc(N(c3cccc(-c4ccc5c(c4)-c4cccc6cccc(c46)S5)c3)c3ccc4c(c3)C(C)(c3ccccc3)c3ccccc3-4)cc2)ccc1-c1ccccc1 ; CC1(c2ccc(N(c3ccc(-c4ccc(-c5ccccc5)c(-c5ccccc5)c4)cc3)c3cccc(-c4ccc5c6c(cccc46)-c4ccccc4O5)c3)cc2)c2ccccc2-c2ccccc21 ; CC1(c2ccc(N(c3ccc(-c4ccc(-c5ccccc5)c(-c5ccccc5)c4)cc3)c3cccc(-c4ccc5c(c4)-c4cccc6cccc(c46)O5)c3)cc2)c2ccccc2-c2ccccc21]

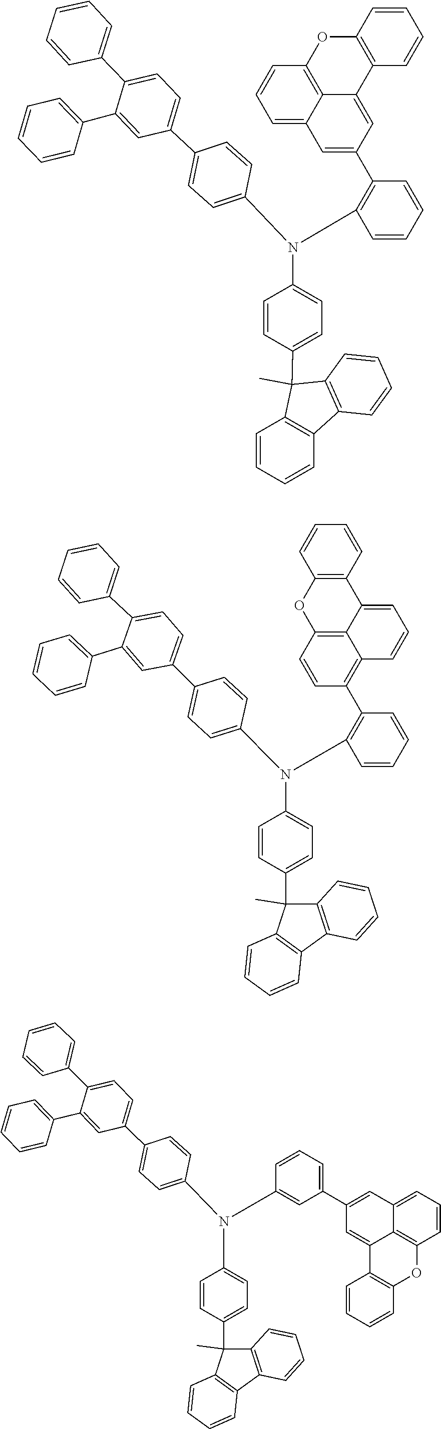 [SMILES: CC1(c2ccc(N(c3ccc(-c4ccc(-c5ccccc5)c(-c5ccccc5)c4)cc3)c3ccccc3-c3cc4c5c(cccc5c3)Oc3ccccc3-4)cc2)c2ccccc2-c2ccccc21 ; CC1(c2ccc(N(c3ccc(-c4ccc(-c5ccccc5)c(-c5ccccc5)c4)cc3)c3ccccc3-c3ccc4c5c(cccc35)-c3ccccc3O4)cc2)c2ccccc2-c2ccccc21 ; CC1(c2ccc(N(c3ccc(-c4ccc(-c5ccccc5)c(-c5ccccc5)c4)cc3)c3cccc(-c4cc5c6c(cccc6c4)Oc4ccccc4-5)c3)cc2)c2ccccc2-c2ccccc21]

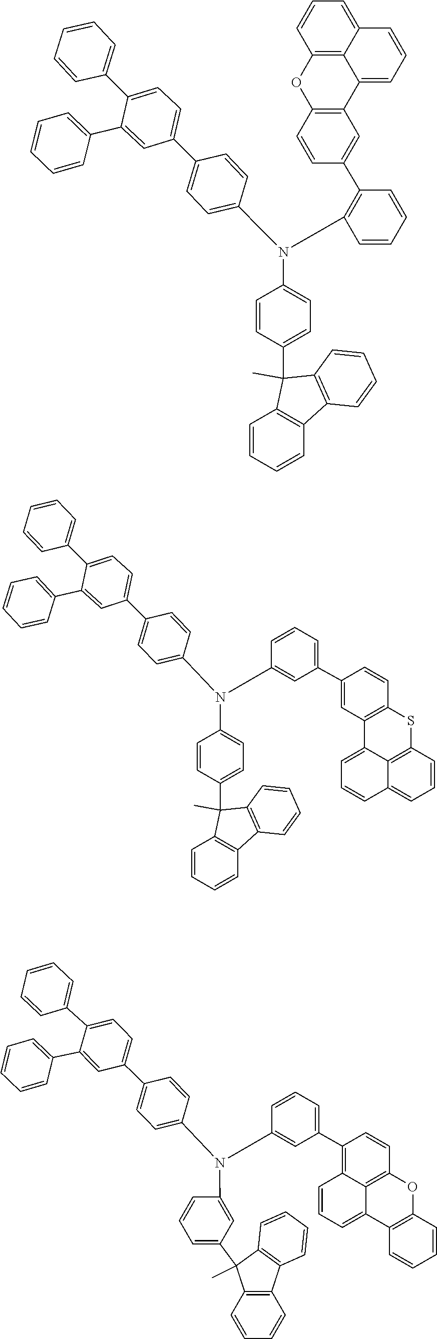 [SMILES: CC1(c2ccc(N(c3ccc(-c4ccc(-c5ccccc5)c(-c5ccccc5)c4)cc3)c3ccccc3-c3ccc4c(c3)-c3cccc5cccc(c35)O4)cc2)c2ccccc2-c2ccccc21 ; CC1(c2ccc(N(c3ccc(-c4ccc(-c5ccccc5)c(-c5ccccc5)c4)cc3)c3cccc(-c4ccc5c(c4)-c4cccc6cccc(c46)S5)c3)cc2)c2ccccc2-c2ccccc21 ; CC1(c2cccc(N(c3ccc(-c4ccc(-c5ccccc5)c(-c5ccccc5)c4)cc3)c3cccc(-c4ccc5c6c(cccc46)-c4ccccc4O5)c3)c2)c2ccccc2-c2ccccc21]

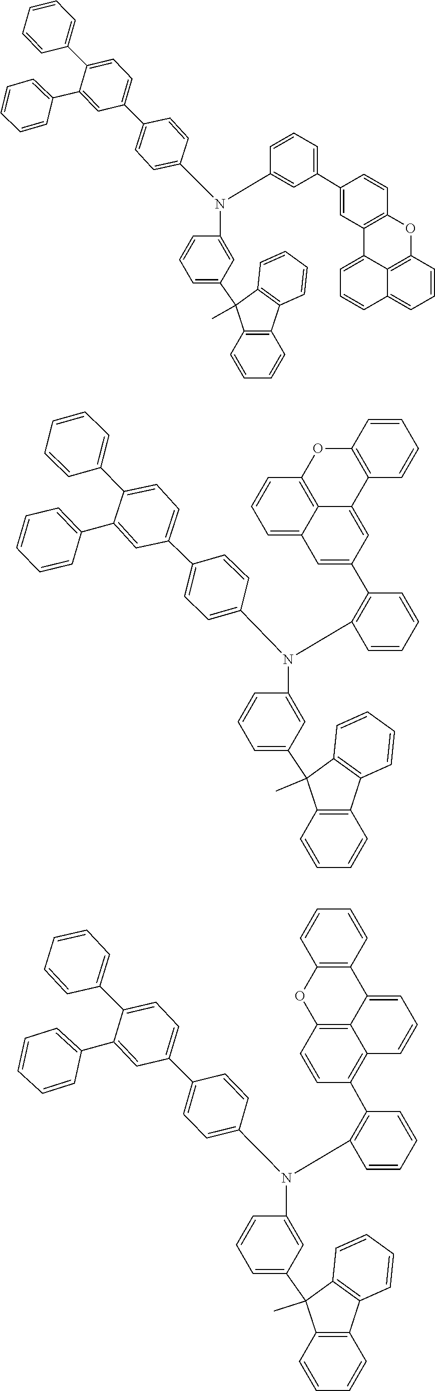

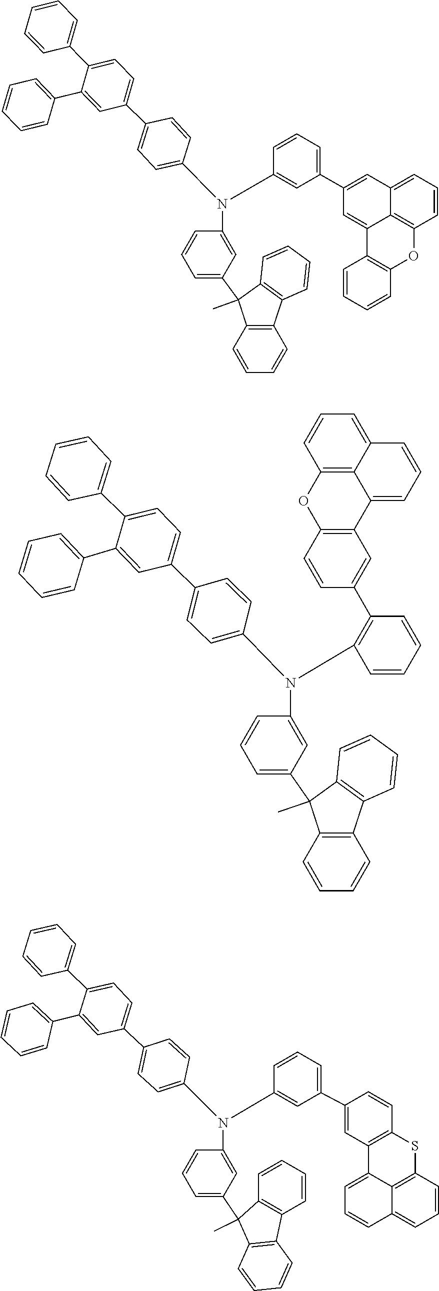 [SMILES: CC1(c2cccc(N(c3ccc(-c4ccc(-c5ccccc5)c(-c5ccccc5)c4)cc3)c3cccc(-c4cc5c6c(cccc6c4)Oc4ccccc4-5)c3)c2)c2ccccc2-c2ccccc21 ; CC1(c2cccc(N(c3ccc(-c4ccc(-c5ccccc5)c(-c5ccccc5)c4)cc3)c3ccccc3-c3ccc4c(c3)-c3cccc5cccc(c35)O4)c2)c2ccccc2-c2ccccc21 ; CC1(c2cccc(N(c3ccc(-c4ccc(-c5ccccc5)c(-c5ccccc5)c4)cc3)c3cccc(-c4ccc5c(c4)-c4cccc6cccc(c46)S5)c3)c2)c2ccccc2-c2ccccc21]

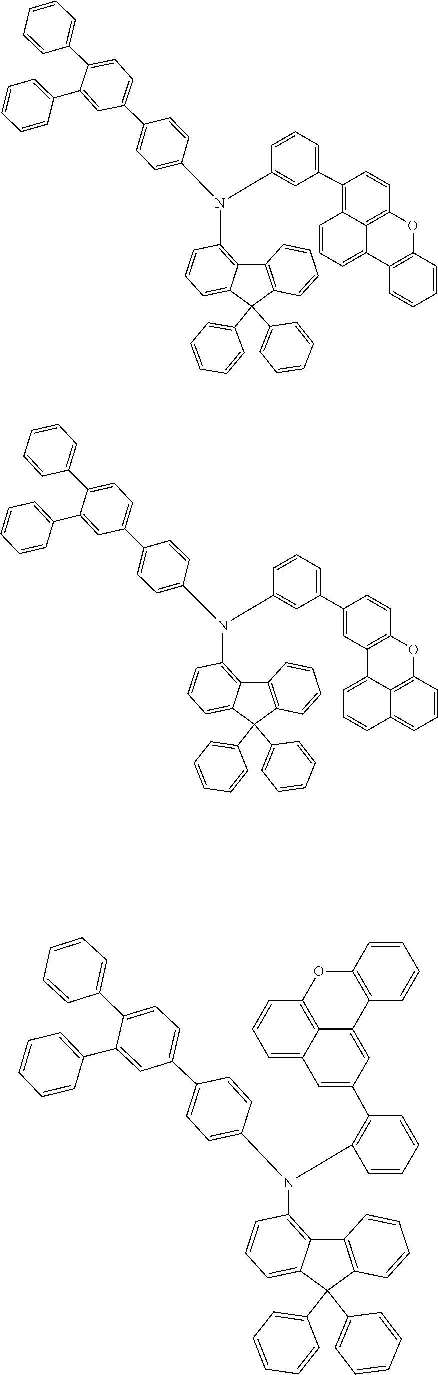 [SMILES: c1ccc(-c2ccc(-c3ccc(N(c4cccc(-c5ccc6c7c(cccc57)-c5ccccc5O6)c4)c4cccc5c4-c4ccccc4C5(c4ccccc4)c4ccccc4)cc3)cc2-c2ccccc2)cc1 ; c1ccc(-c2ccc(-c3ccc(N(c4cccc(-c5ccc6c(c5)-c5cccc7cccc(c57)O6)c4)c4cccc5c4-c4ccccc4C5(c4ccccc4)c4ccccc4)cc3)cc2-c2ccccc2)cc1 ; c1ccc(-c2ccc(-c3ccc(N(c4ccccc4-c4cc5c6c(cccc6c4)Oc4ccccc4-5)c4cccc5c4-c4ccccc4C5(c4ccccc4)c4ccccc4)cc3)cc2-c2ccccc2)cc1]

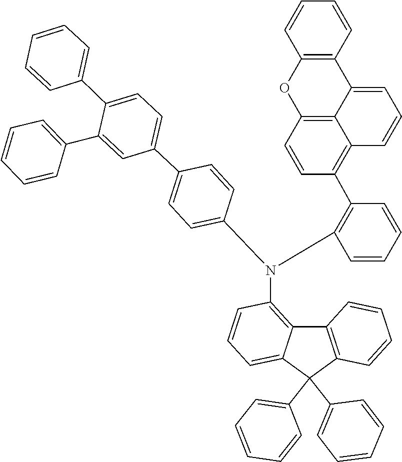 [SMILES: c1ccc(-c2ccc(-c3ccc(N(c4ccccc4-c4ccc5c6c(cccc46)-c4ccccc4O5)c4cccc5c4-c4ccccc4C5(c4ccccc4)c4ccccc4)cc3)cc2-c2ccccc2)cc1]

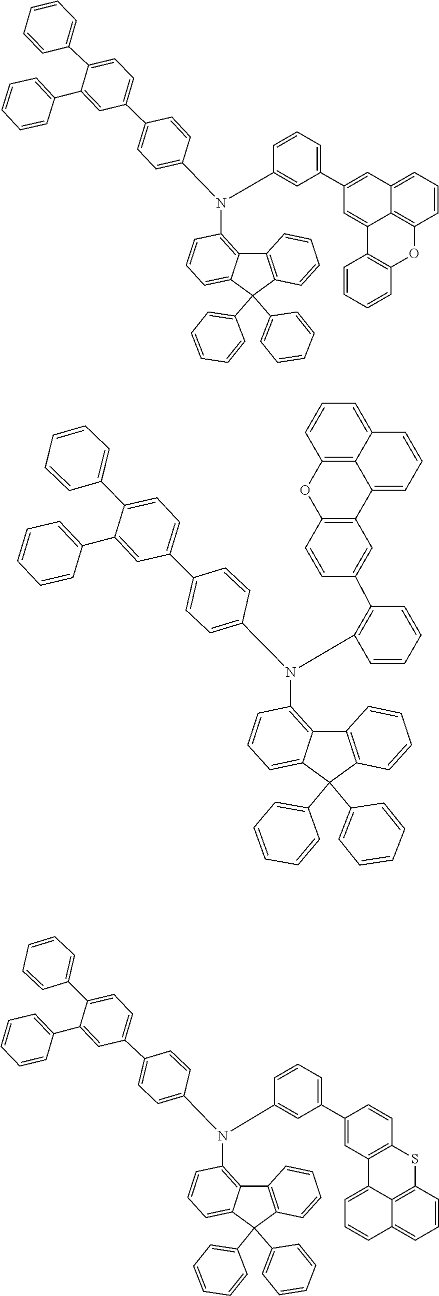 [SMILES: c1ccc(-c2ccc(-c3ccc(N(c4cccc(-c5cc6c7c(cccc7c5)Oc5ccccc5-6)c4)c4cccc5c4-c4ccccc4C5(c4ccccc4)c4ccccc4)cc3)cc2-c2ccccc2)cc1 ; c1ccc(-c2ccc(-c3ccc(N(c4ccccc4-c4ccc5c(c4)-c4cccc6cccc(c46)O5)c4cccc5c4-c4ccccc4C5(c4ccccc4)c4ccccc4)cc3)cc2-c2ccccc2)cc1 ; c1ccc(-c2ccc(-c3ccc(N(c4cccc(-c5ccc6c(c5)-c5cccc7cccc(c57)S6)c4)c4cccc5c4-c4ccccc4C5(c4ccccc4)c4ccccc4)cc3)cc2-c2ccccc2)cc1]

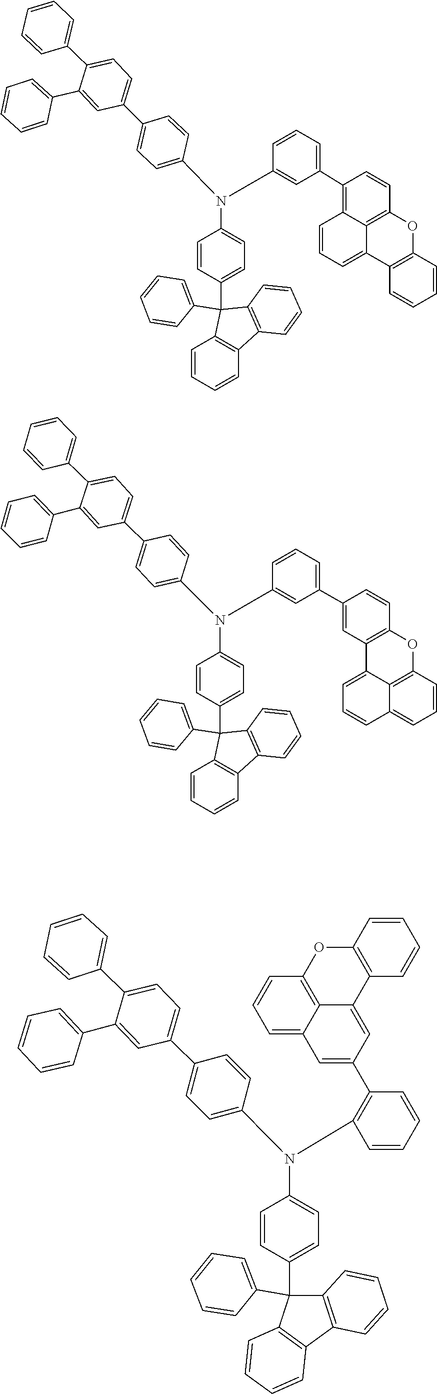 [SMILES: c1ccc(-c2ccc(-c3ccc(N(c4ccc(C5(c6ccccc6)c6ccccc6-c6ccccc65)cc4)c4cccc(-c5ccc6c7c(cccc57)-c5ccccc5O6)c4)cc3)cc2-c2ccccc2)cc1 ; c1ccc(-c2ccc(-c3ccc(N(c4ccc(C5(c6ccccc6)c6ccccc6-c6ccccc65)cc4)c4cccc(-c5ccc6c(c5)-c5cccc7cccc(c57)O6)c4)cc3)cc2-c2ccccc2)cc1 ; c1ccc(-c2ccc(-c3ccc(N(c4ccc(C5(c6ccccc6)c6ccccc6-c6ccccc65)cc4)c4ccccc4-c4cc5c6c(cccc6c4)Oc4ccccc4-5)cc3)cc2-c2ccccc2)cc1]

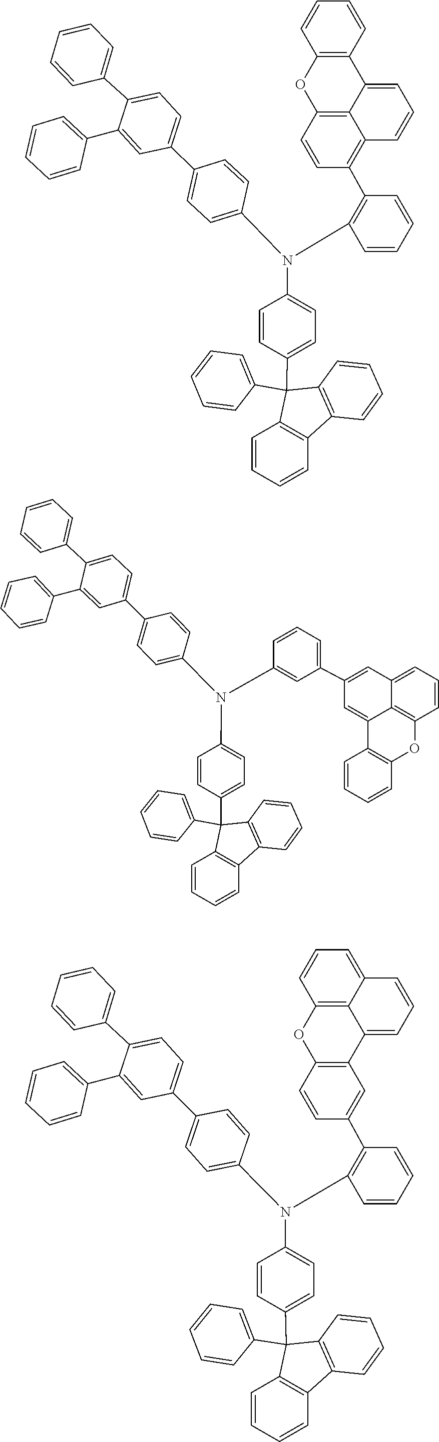 [SMILES: c1ccc(-c2ccc(-c3ccc(N(c4ccc(C5(c6ccccc6)c6ccccc6-c6ccccc65)cc4)c4ccccc4-c4ccc5c6c(cccc46)-c4ccccc4O5)cc3)cc2-c2ccccc2)cc1 ; c1ccc(-c2ccc(-c3ccc(N(c4ccc(C5(c6ccccc6)c6ccccc6-c6ccccc65)cc4)c4cccc(-c5cc6c7c(cccc7c5)Oc5ccccc5-6)c4)cc3)cc2-c2ccccc2)cc1 ; c1ccc(-c2ccc(-c3ccc(N(c4ccc(C5(c6ccccc6)c6ccccc6-c6ccccc65)cc4)c4ccccc4-c4ccc5c(c4)-c4cccc6cccc(c46)O5)cc3)cc2-c2ccccc2)cc1]

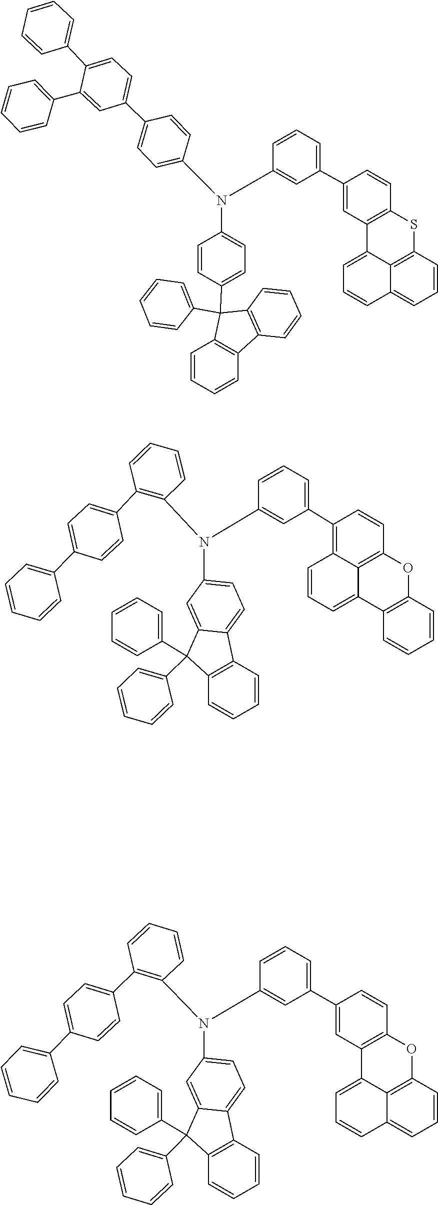 [SMILES: c1ccc(-c2ccc(-c3ccc(N(c4ccc(C5(c6ccccc6)c6ccccc6-c6ccccc65)cc4)c4cccc(-c5ccc6c(c5)-c5cccc7cccc(c57)S6)c4)cc3)cc2-c2ccccc2)cc1 ; c1ccc(-c2ccc(-c3ccccc3N(c3cccc(-c4ccc5c6c(cccc46)-c4ccccc4O5)c3)c3ccc4c(c3)C(c3ccccc3)(c3ccccc3)c3ccccc3-4)cc2)cc1 ; c1ccc(-c2ccc(-c3ccccc3N(c3cccc(-c4ccc5c(c4)-c4cccc6cccc(c46)O5)c3)c3ccc4c(c3)C(c3ccccc3)(c3ccccc3)c3ccccc3-4)cc2)cc1]

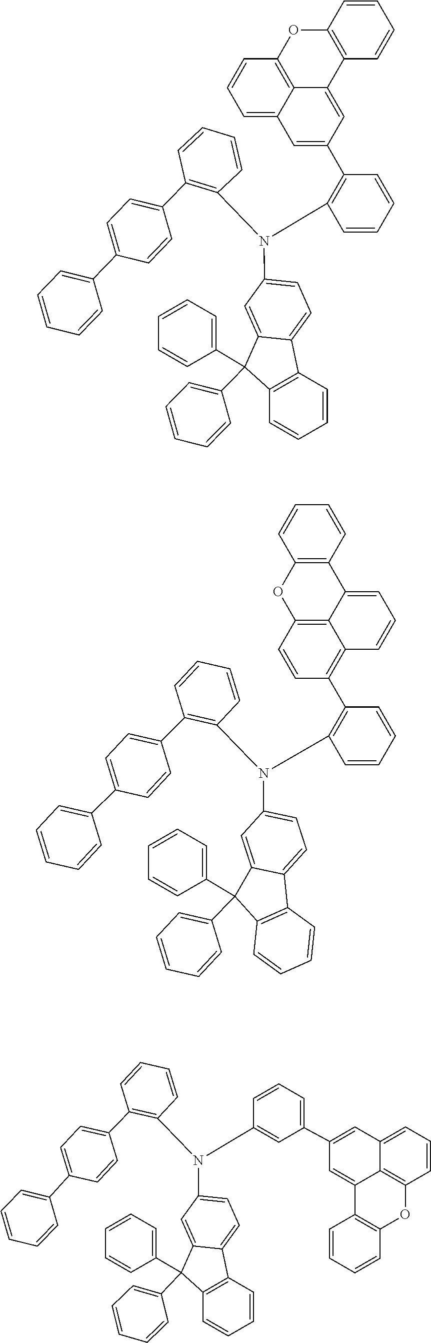 [SMILES: c1ccc(-c2ccc(-c3ccccc3N(c3ccc4c(c3)C(c3ccccc3)(c3ccccc3)c3ccccc3-4)c3ccccc3-c3cc4c5c(cccc5c3)Oc3ccccc3-4)cc2)cc1 ; c1ccc(-c2ccc(-c3ccccc3N(c3ccc4c(c3)C(c3ccccc3)(c3ccccc3)c3ccccc3-4)c3ccccc3-c3ccc4c5c(cccc35)-c3ccccc3O4)cc2)cc1 ; c1ccc(-c2ccc(-c3ccccc3N(c3cccc(-c4cc5c6c(cccc6c4)Oc4ccccc4-5)c3)c3ccc4c(c3)C(c3ccccc3)(c3ccccc3)c3ccccc3-4)cc2)cc1]

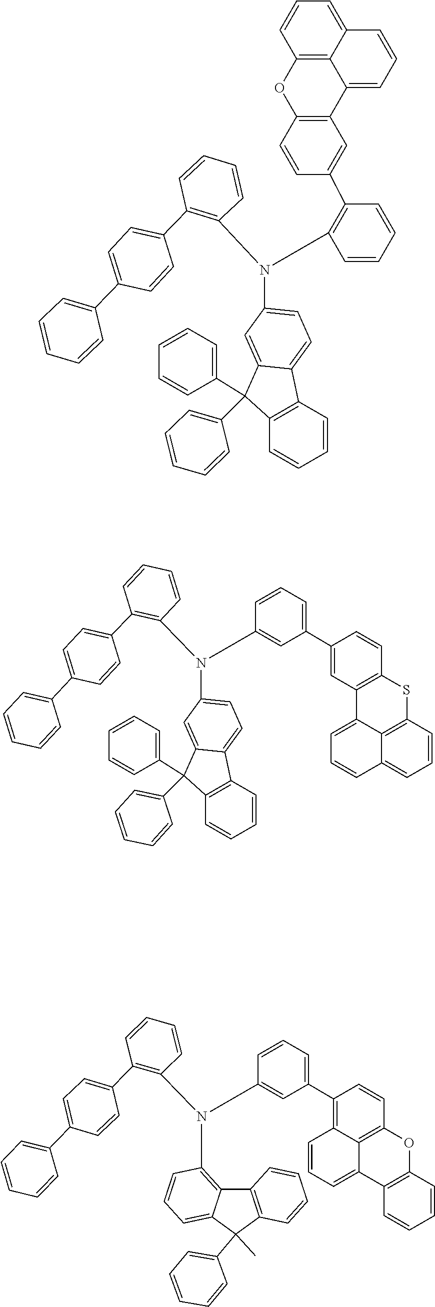 [SMILES: c1ccc(-c2ccc(-c3ccccc3N(c3ccc4c(c3)C(c3ccccc3)(c3ccccc3)c3ccccc3-4)c3ccccc3-c3ccc4c(c3)-c3cccc5cccc(c35)O4)cc2)cc1 ; c1ccc(-c2ccc(-c3ccccc3N(c3cccc(-c4ccc5c(c4)-c4cccc6cccc(c46)S5)c3)c3ccc4c(c3)C(c3ccccc3)(c3ccccc3)c3ccccc3-4)cc2)cc1 ; CC1(c2ccccc2)c2ccccc2-c2c(N(c3cccc(-c4ccc5c6c(cccc46)-c4ccccc4O5)c3)c3ccccc3-c3ccc(-c4ccccc4)cc3)cccc21]

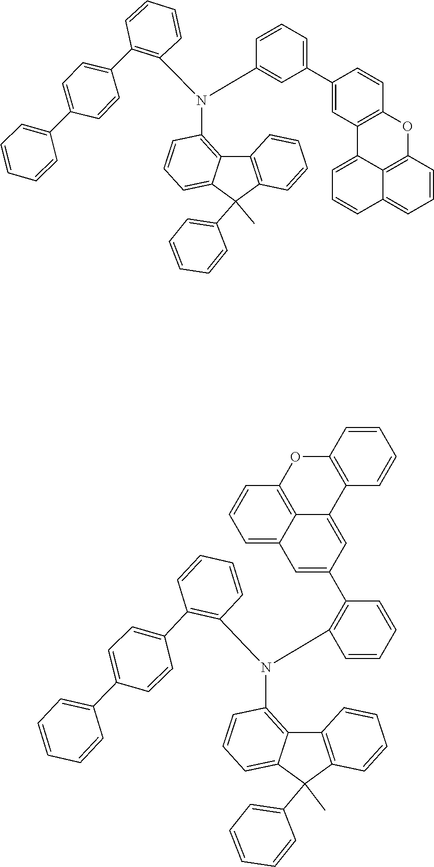 [SMILES: CC1(c2ccccc2)c2ccccc2-c2c(N(c3cccc(-c4ccc5c(c4)-c4cccc6cccc(c46)O5)c3)c3ccccc3-c3ccc(-c4ccccc4)cc3)cccc21 ; CC1(c2ccccc2)c2ccccc2-c2c(N(c3ccccc3-c3ccc(-c4ccccc4)cc3)c3ccccc3-c3cc4c5c(cccc5c3)Oc3ccccc3-4)cccc21]

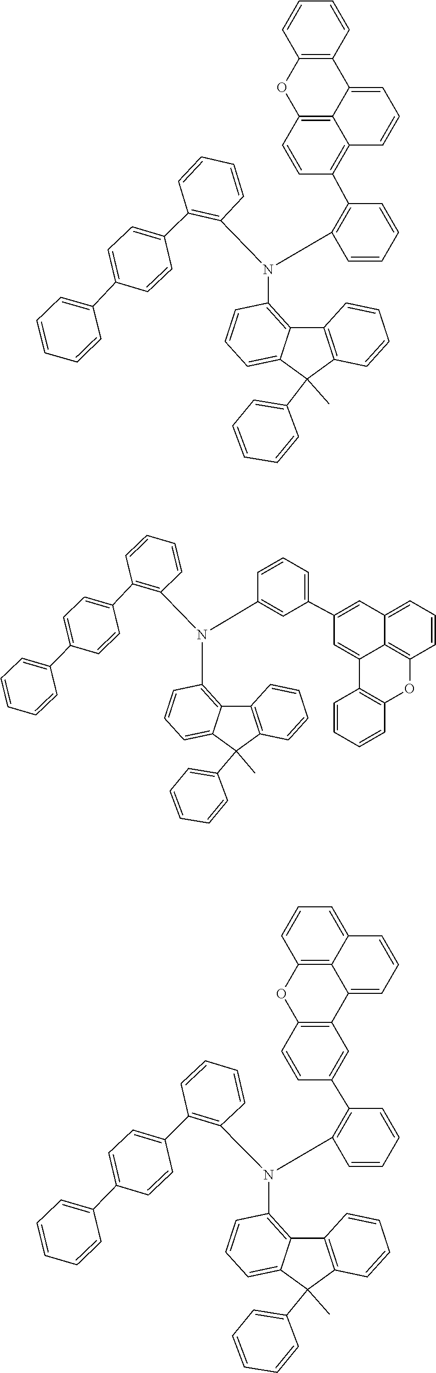 [SMILES: CC1(c2ccccc2)c2ccccc2-c2c(N(c3ccccc3-c3ccc(-c4ccccc4)cc3)c3ccccc3-c3ccc4c5c(cccc35)-c3ccccc3O4)cccc21 ; CC1(c2ccccc2)c2ccccc2-c2c(N(c3cccc(-c4cc5c6c(cccc6c4)Oc4ccccc4-5)c3)c3ccccc3-c3ccc(-c4ccccc4)cc3)cccc21 ; CC1(c2ccccc2)c2ccccc2-c2c(N(c3ccccc3-c3ccc(-c4ccccc4)cc3)c3ccccc3-c3ccc4c(c3)-c3cccc5cccc(c35)O4)cccc21]

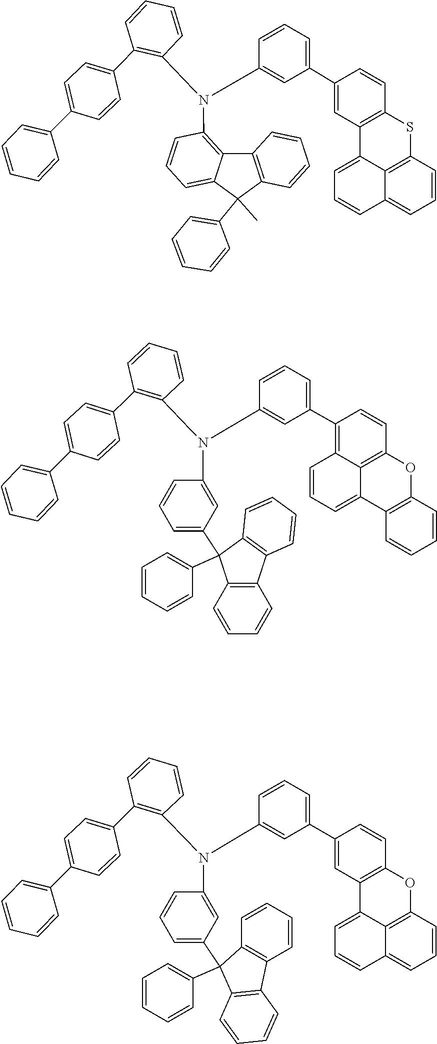 [SMILES: CC1(c2ccccc2)c2ccccc2-c2c(N(c3cccc(-c4ccc5c(c4)-c4cccc6cccc(c46)S5)c3)c3ccccc3-c3ccc(-c4ccccc4)cc3)cccc21 ; c1ccc(-c2ccc(-c3ccccc3N(c3cccc(-c4ccc5c6c(cccc46)-c4ccccc4O5)c3)c3cccc(C4(c5ccccc5)c5ccccc5-c5ccccc54)c3)cc2)cc1 ; c1ccc(-c2ccc(-c3ccccc3N(c3cccc(-c4ccc5c(c4)-c4cccc6cccc(c46)O5)c3)c3cccc(C4(c5ccccc5)c5ccccc5-c5ccccc54)c3)cc2)cc1]

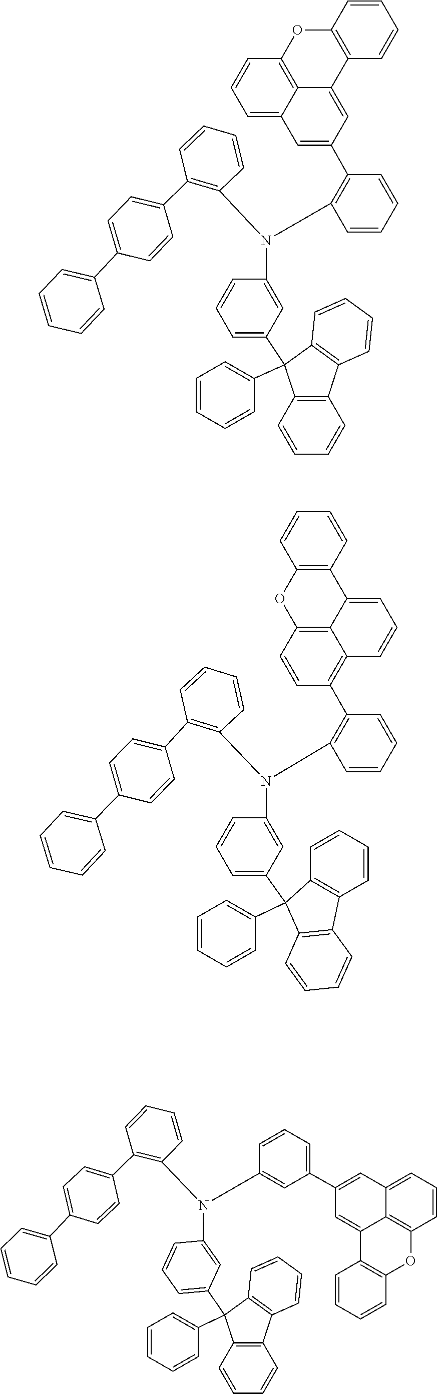 [SMILES: c1ccc(-c2ccc(-c3ccccc3N(c3cccc(C4(c5ccccc5)c5ccccc5-c5ccccc54)c3)c3ccccc3-c3cc4c5c(cccc5c3)Oc3ccccc3-4)cc2)cc1 ; c1ccc(-c2ccc(-c3ccccc3N(c3cccc(C4(c5ccccc5)c5ccccc5-c5ccccc54)c3)c3ccccc3-c3ccc4c5c(cccc35)-c3ccccc3O4)cc2)cc1 ; c1ccc(-c2ccc(-c3ccccc3N(c3cccc(-c4cc5c6c(cccc6c4)Oc4ccccc4-5)c3)c3cccc(C4(c5ccccc5)c5ccccc5-c5ccccc54)c3)cc2)cc1]

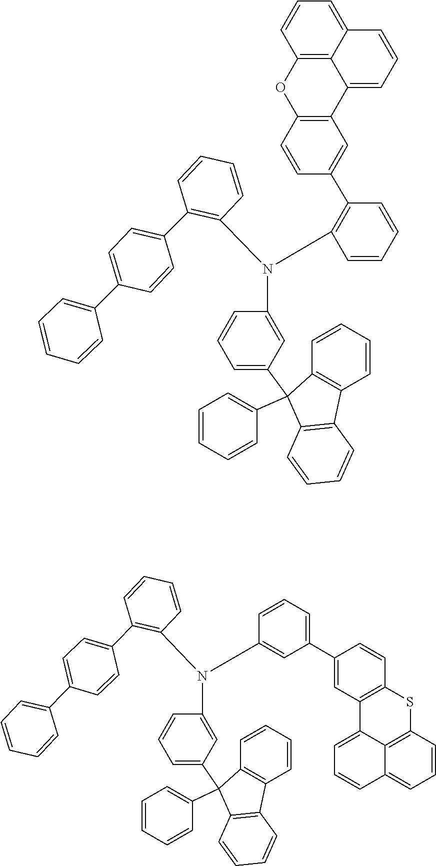 [SMILES: c1ccc(-c2ccc(-c3ccccc3N(c3cccc(C4(c5ccccc5)c5ccccc5-c5ccccc54)c3)c3ccccc3-c3ccc4c(c3)-c3cccc5cccc(c35)O4)cc2)cc1 ; c1ccc(-c2ccc(-c3ccccc3N(c3cccc(-c4ccc5c(c4)-c4cccc6cccc(c46)S5)c3)c3cccc(C4(c5ccccc5)c5ccccc5-c5ccccc54)c3)cc2)cc1]

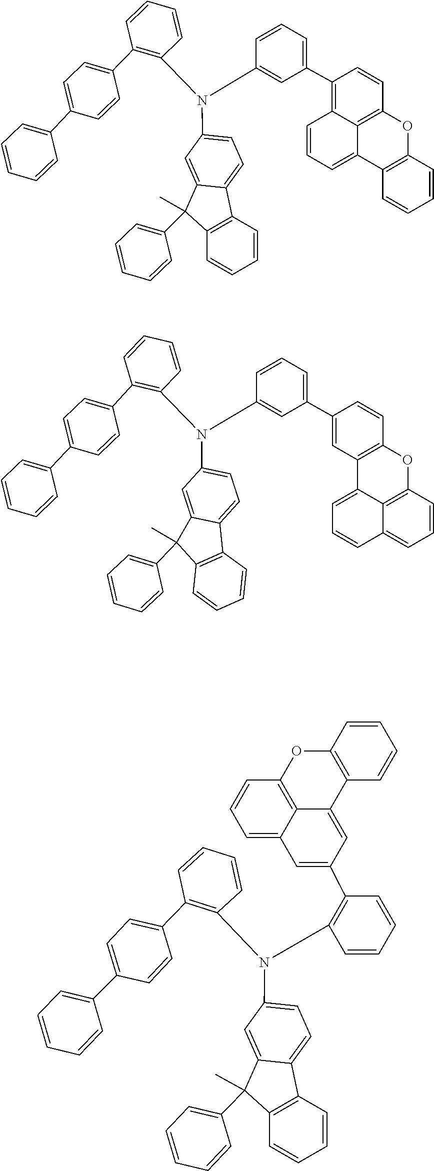 [SMILES: CC1(c2ccccc2)c2ccccc2-c2ccc(N(c3cccc(-c4ccc5c6c(cccc46)-c4ccccc4O5)c3)c3ccccc3-c3ccc(-c4ccccc4)cc3)cc21 ; CC1(c2ccccc2)c2ccccc2-c2ccc(N(c3cccc(-c4ccc5c(c4)-c4cccc6cccc(c46)O5)c3)c3ccccc3-c3ccc(-c4ccccc4)cc3)cc21 ; CC1(c2ccccc2)c2ccccc2-c2ccc(N(c3ccccc3-c3ccc(-c4ccccc4)cc3)c3ccccc3-c3cc4c5c(cccc5c3)Oc3ccccc3-4)cc21]

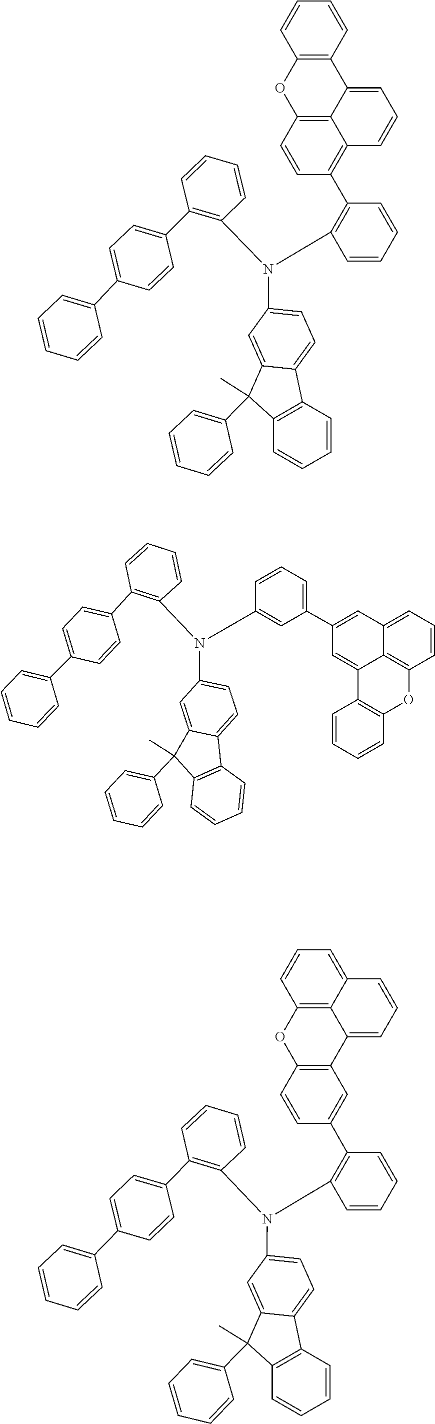 [SMILES: CC1(c2ccccc2)c2ccccc2-c2ccc(N(c3ccccc3-c3ccc(-c4ccccc4)cc3)c3ccccc3-c3ccc4c5c(cccc35)-c3ccccc3O4)cc21 ; CC1(c2ccccc2)c2ccccc2-c2ccc(N(c3cccc(-c4cc5c6c(cccc6c4)Oc4ccccc4-5)c3)c3ccccc3-c3ccc(-c4ccccc4)cc3)cc21 ; CC1(c2ccccc2)c2ccccc2-c2ccc(N(c3ccccc3-c3ccc(-c4ccccc4)cc3)c3ccccc3-c3ccc4c(c3)-c3cccc5cccc(c35)O4)cc21]

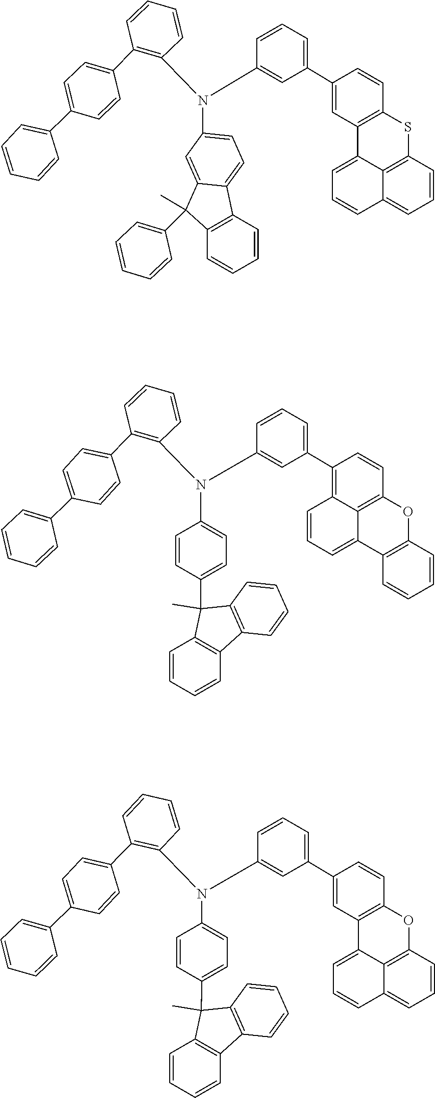 [SMILES: CC1(c2ccccc2)c2ccccc2-c2ccc(N(c3cccc(-c4ccc5c(c4)-c4cccc6cccc(c46)S5)c3)c3ccccc3-c3ccc(-c4ccccc4)cc3)cc21 ; CC1(c2ccc(N(c3cccc(-c4ccc5c6c(cccc46)-c4ccccc4O5)c3)c3ccccc3-c3ccc(-c4ccccc4)cc3)cc2)c2ccccc2-c2ccccc21 ; CC1(c2ccc(N(c3cccc(-c4ccc5c(c4)-c4cccc6cccc(c46)O5)c3)c3ccccc3-c3ccc(-c4ccccc4)cc3)cc2)c2ccccc2-c2ccccc21]

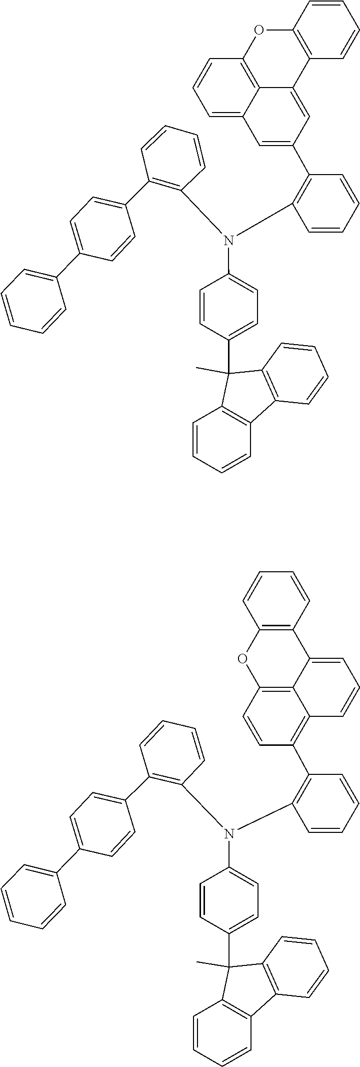 [SMILES: CC1(c2ccc(N(c3ccccc3-c3ccc(-c4ccccc4)cc3)c3ccccc3-c3cc4c5c(cccc5c3)Oc3ccccc3-4)cc2)c2ccccc2-c2ccccc21 ; CC1(c2ccc(N(c3ccccc3-c3ccc(-c4ccccc4)cc3)c3ccccc3-c3ccc4c5c(cccc35)-c3ccccc3O4)cc2)c2ccccc2-c2ccccc21]

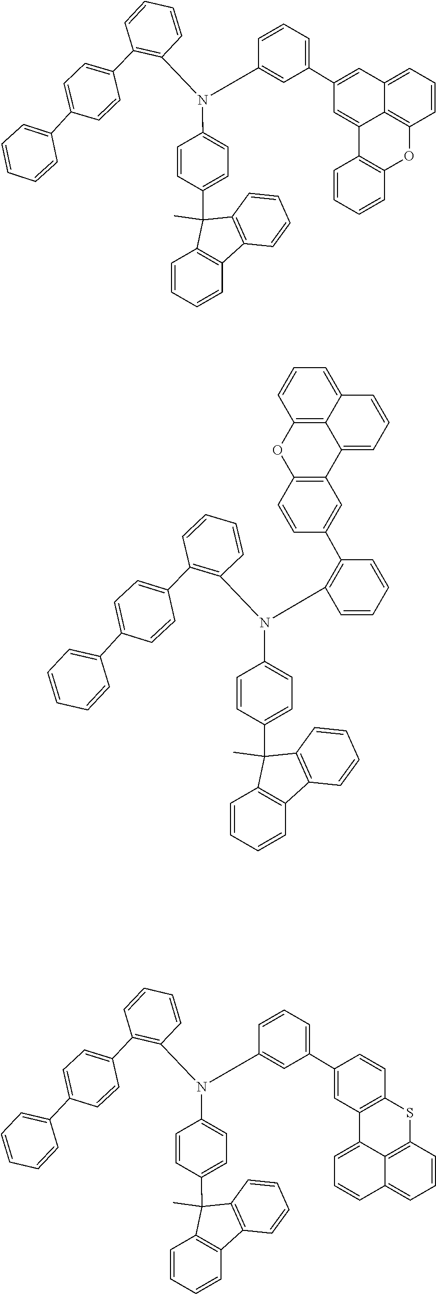 [SMILES: CC1(c2ccc(N(c3cccc(-c4cc5c6c(cccc6c4)Oc4ccccc4-5)c3)c3ccccc3-c3ccc(-c4ccccc4)cc3)cc2)c2ccccc2-c2ccccc21 ; CC1(c2ccc(N(c3ccccc3-c3ccc(-c4ccccc4)cc3)c3ccccc3-c3ccc4c(c3)-c3cccc5cccc(c35)O4)cc2)c2ccccc2-c2ccccc21 ; CC1(c2ccc(N(c3cccc(-c4ccc5c(c4)-c4cccc6cccc(c46)S5)c3)c3ccccc3-c3ccc(-c4ccccc4)cc3)cc2)c2ccccc2-c2ccccc21]

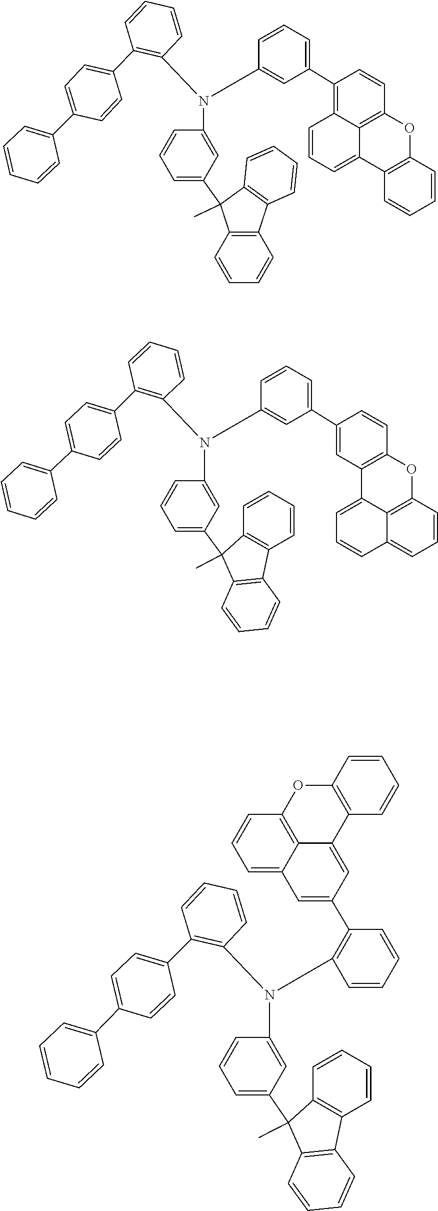 [SMILES: CC1(c2cccc(N(c3cccc(-c4ccc5c6c(cccc46)-c4ccccc4O5)c3)c3ccccc3-c3ccc(-c4ccccc4)cc3)c2)c2ccccc2-c2ccccc21 ; CC1(c2cccc(N(c3cccc(-c4ccc5c(c4)-c4cccc6cccc(c46)O5)c3)c3ccccc3-c3ccc(-c4ccccc4)cc3)c2)c2ccccc2-c2ccccc21 ; CC1(c2cccc(N(c3ccccc3-c3ccc(-c4ccccc4)cc3)c3ccccc3-c3cc4c5c(cccc5c3)Oc3ccccc3-4)c2)c2ccccc2-c2ccccc21]

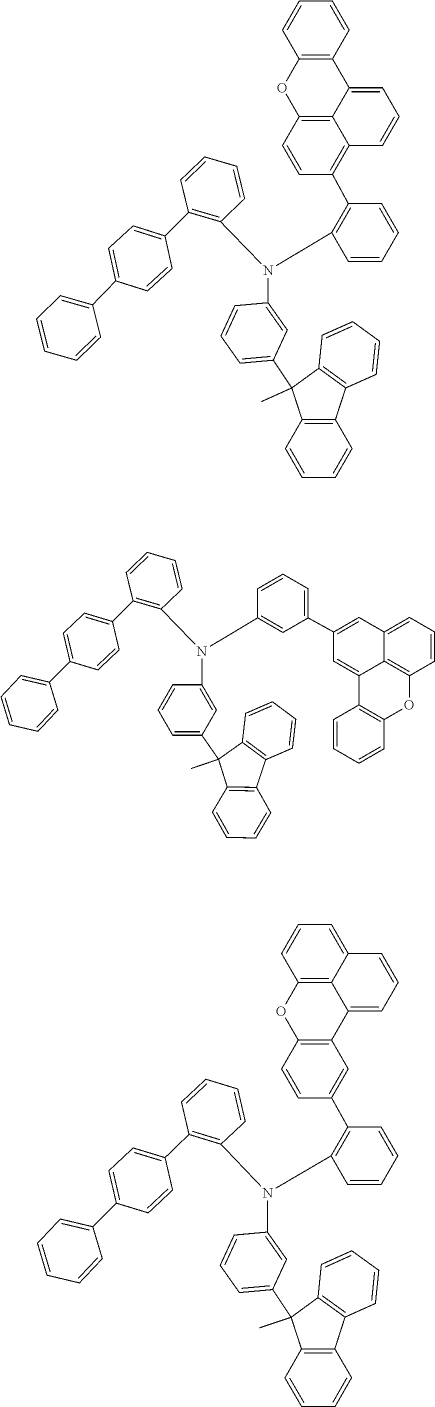 [SMILES: CC1(c2cccc(N(c3ccccc3-c3ccc(-c4ccccc4)cc3)c3ccccc3-c3ccc4c5c(cccc35)-c3ccccc3O4)c2)c2ccccc2-c2ccccc21 ; CC1(c2cccc(N(c3cccc(-c4cc5c6c(cccc6c4)Oc4ccccc4-5)c3)c3ccccc3-c3ccc(-c4ccccc4)cc3)c2)c2ccccc2-c2ccccc21 ; CC1(c2cccc(N(c3ccccc3-c3ccc(-c4ccccc4)cc3)c3ccccc3-c3ccc4c(c3)-c3cccc5cccc(c35)O4)c2)c2ccccc2-c2ccccc21]

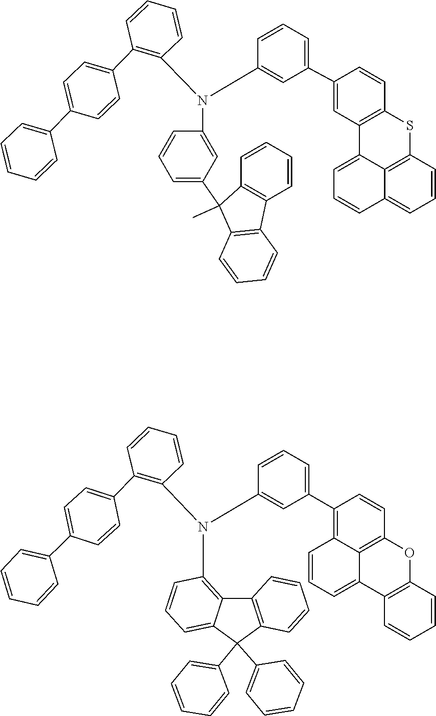 [SMILES: CC1(c2cccc(N(c3cccc(-c4ccc5c(c4)-c4cccc6cccc(c46)S5)c3)c3ccccc3-c3ccc(-c4ccccc4)cc3)c2)c2ccccc2-c2ccccc21 ; c1ccc(-c2ccc(-c3ccccc3N(c3cccc(-c4ccc5c6c(cccc46)-c4ccccc4O5)c3)c3cccc4c3-c3ccccc3C4(c3ccccc3)c3ccccc3)cc2)cc1]

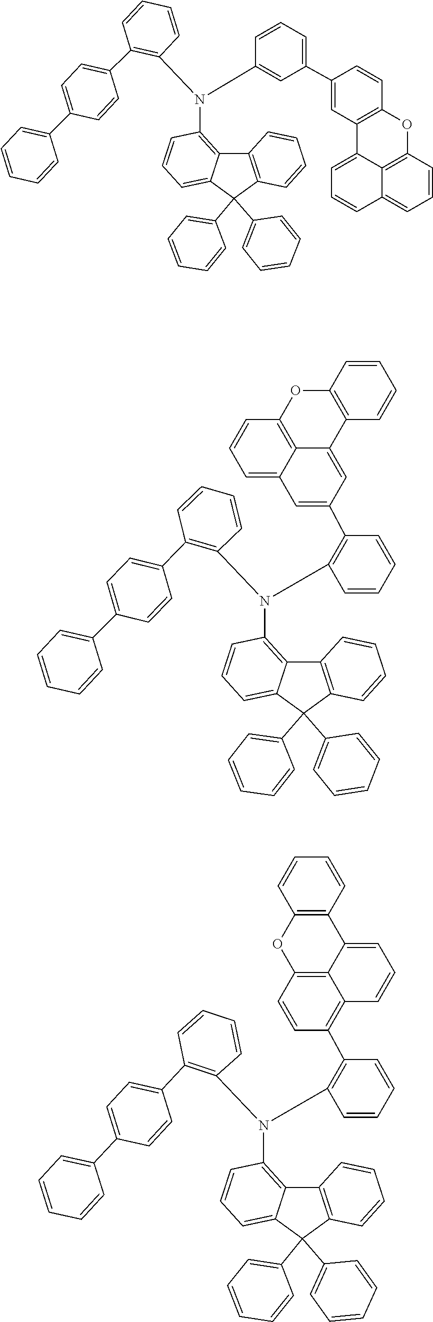 [SMILES: c1ccc(-c2ccc(-c3ccccc3N(c3cccc(-c4ccc5c(c4)-c4cccc6cccc(c46)O5)c3)c3cccc4c3-c3ccccc3C4(c3ccccc3)c3ccccc3)cc2)cc1 ; c1ccc(-c2ccc(-c3ccccc3N(c3ccccc3-c3cc4c5c(cccc5c3)Oc3ccccc3-4)c3cccc4c3-c3ccccc3C4(c3ccccc3)c3ccccc3)cc2)cc1 ; c1ccc(-c2ccc(-c3ccccc3N(c3ccccc3-c3ccc4c5c(cccc35)-c3ccccc3O4)c3cccc4c3-c3ccccc3C4(c3ccccc3)c3ccccc3)cc2)cc1]

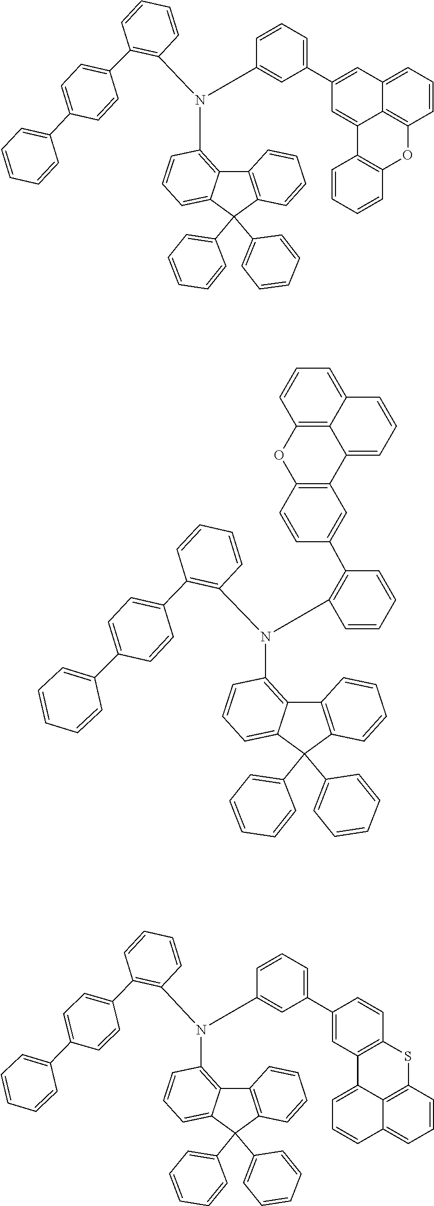 [SMILES: c1ccc(-c2ccc(-c3ccccc3N(c3cccc(-c4cc5c6c(cccc6c4)Oc4ccccc4-5)c3)c3cccc4c3-c3ccccc3C4(c3ccccc3)c3ccccc3)cc2)cc1 ; c1ccc(-c2ccc(-c3ccccc3N(c3ccccc3-c3ccc4c(c3)-c3cccc5cccc(c35)O4)c3cccc4c3-c3ccccc3C4(c3ccccc3)c3ccccc3)cc2)cc1 ; c1ccc(-c2ccc(-c3ccccc3N(c3cccc(-c4ccc5c(c4)-c4cccc6cccc(c46)S5)c3)c3cccc4c3-c3ccccc3C4(c3ccccc3)c3ccccc3)cc2)cc1]

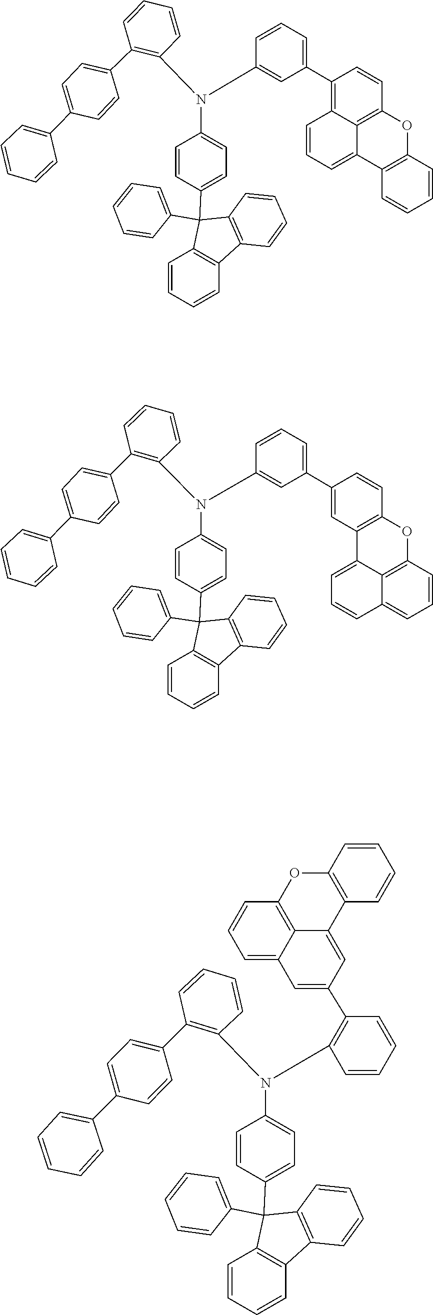 [SMILES: c1ccc(-c2ccc(-c3ccccc3N(c3ccc(C4(c5ccccc5)c5ccccc5-c5ccccc54)cc3)c3cccc(-c4ccc5c6c(cccc46)-c4ccccc4O5)c3)cc2)cc1 ; c1ccc(-c2ccc(-c3ccccc3N(c3ccc(C4(c5ccccc5)c5ccccc5-c5ccccc54)cc3)c3cccc(-c4ccc5c(c4)-c4cccc6cccc(c46)O5)c3)cc2)cc1 ; c1ccc(-c2ccc(-c3ccccc3N(c3ccc(C4(c5ccccc5)c5ccccc5-c5ccccc54)cc3)c3ccccc3-c3cc4c5c(cccc5c3)Oc3ccccc3-4)cc2)cc1]

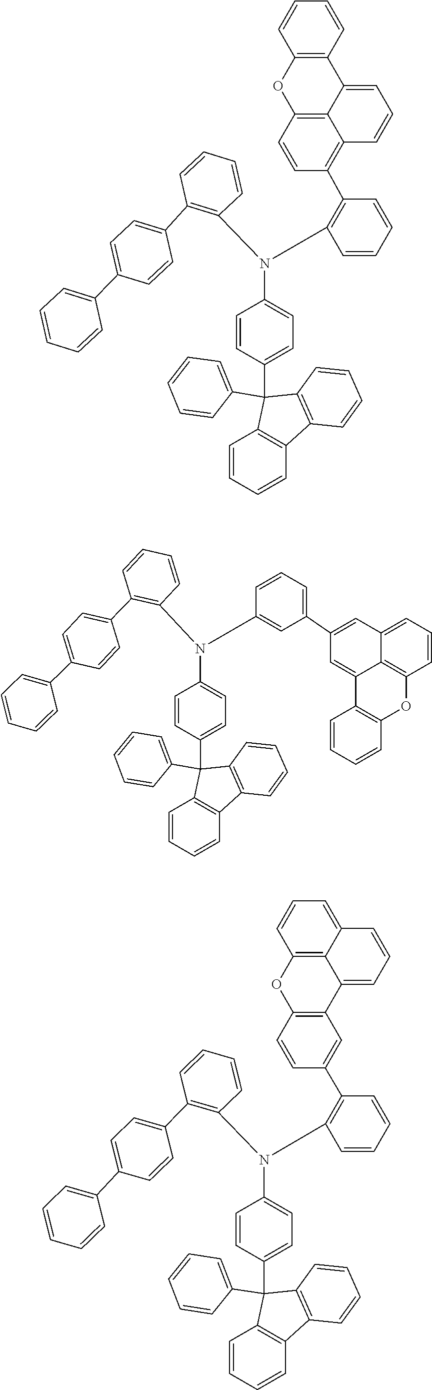 [SMILES: c1ccc(-c2ccc(-c3ccccc3N(c3ccc(C4(c5ccccc5)c5ccccc5-c5ccccc54)cc3)c3ccccc3-c3ccc4c5c(cccc35)-c3ccccc3O4)cc2)cc1 ; c1ccc(-c2ccc(-c3ccccc3N(c3ccc(C4(c5ccccc5)c5ccccc5-c5ccccc54)cc3)c3cccc(-c4cc5c6c(cccc6c4)Oc4ccccc4-5)c3)cc2)cc1 ; c1ccc(-c2ccc(-c3ccccc3N(c3ccc(C4(c5ccccc5)c5ccccc5-c5ccccc54)cc3)c3ccccc3-c3ccc4c(c3)-c3cccc5cccc(c35)O4)cc2)cc1]

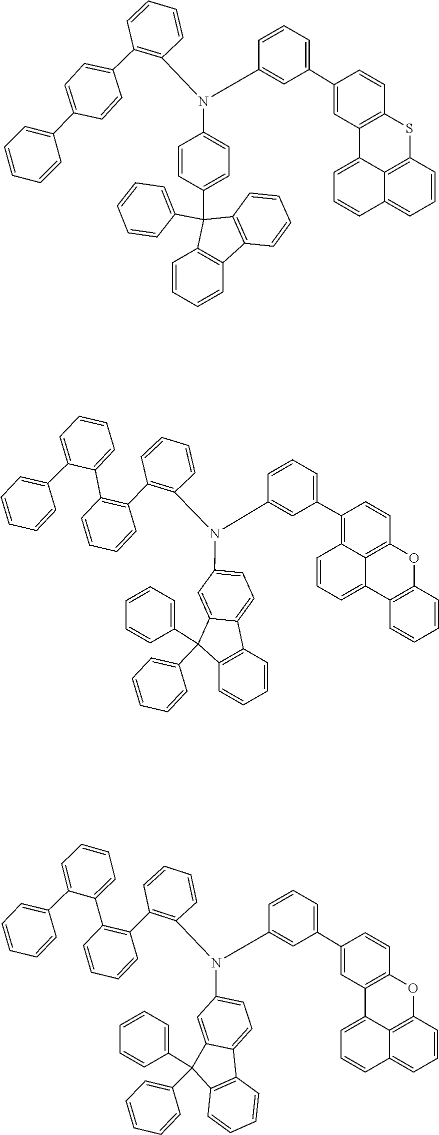 [SMILES: c1ccc(-c2ccc(-c3ccccc3N(c3ccc(C4(c5ccccc5)c5ccccc5-c5ccccc54)cc3)c3cccc(-c4ccc5c(c4)-c4cccc6cccc(c46)S5)c3)cc2)cc1 ; c1ccc(-c2ccccc2-c2ccccc2-c2ccccc2N(c2cccc(-c3ccc4c5c(cccc35)-c3ccccc3O4)c2)c2ccc3c(c2)C(c2ccccc2)(c2ccccc2)c2ccccc2-3)cc1 ; c1ccc(-c2ccccc2-c2ccccc2-c2ccccc2N(c2cccc(-c3ccc4c(c3)-c3cccc5cccc(c35)O4)c2)c2ccc3c(c2)C(c2ccccc2)(c2ccccc2)c2ccccc2-3)cc1]

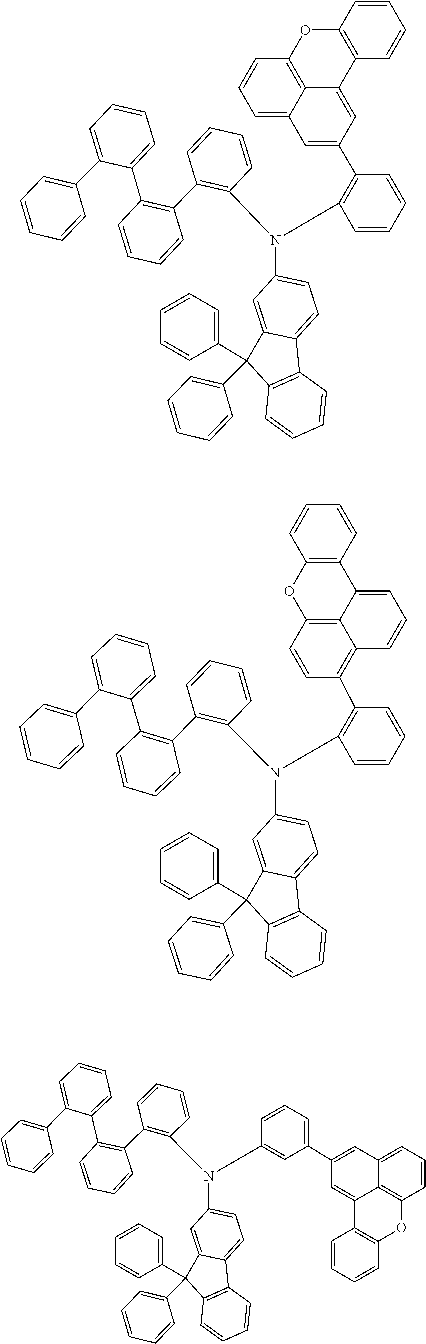 [SMILES: c1ccc(-c2ccccc2-c2ccccc2-c2ccccc2N(c2ccc3c(c2)C(c2ccccc2)(c2ccccc2)c2ccccc2-3)c2ccccc2-c2cc3c4c(cccc4c2)Oc2ccccc2-3)cc1 ; c1ccc(-c2ccccc2-c2ccccc2-c2ccccc2N(c2ccc3c(c2)C(c2ccccc2)(c2ccccc2)c2ccccc2-3)c2ccccc2-c2ccc3c4c(cccc24)-c2ccccc2O3)cc1 ; c1ccc(-c2ccccc2-c2ccccc2-c2ccccc2N(c2cccc(-c3cc4c5c(cccc5c3)Oc3ccccc3-4)c2)c2ccc3c(c2)C(c2ccccc2)(c2ccccc2)c2ccccc2-3)cc1]

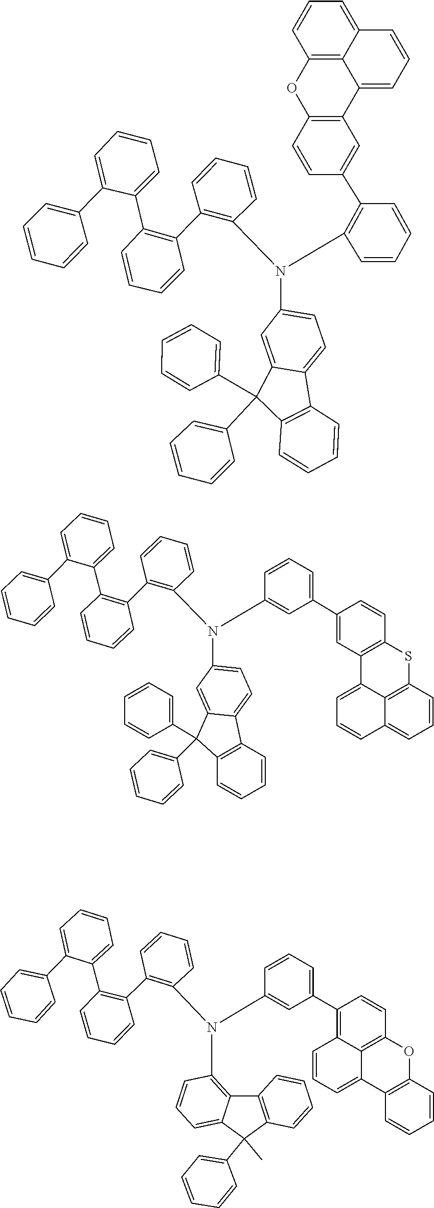 [SMILES: c1ccc(-c2ccccc2-c2ccccc2-c2ccccc2N(c2ccc3c(c2)C(c2ccccc2)(c2ccccc2)c2ccccc2-3)c2ccccc2-c2ccc3c(c2)-c2cccc4cccc(c24)O3)cc1 ; c1ccc(-c2ccccc2-c2ccccc2-c2ccccc2N(c2cccc(-c3ccc4c(c3)-c3cccc5cccc(c35)S4)c2)c2ccc3c(c2)C(c2ccccc2)(c2ccccc2)c2ccccc2-3)cc1 ; CC1(c2ccccc2)c2ccccc2-c2c(N(c3cccc(-c4ccc5c6c(cccc46)-c4ccccc4O5)c3)c3ccccc3-c3ccccc3-c3ccccc3-c3ccccc3)cccc21]

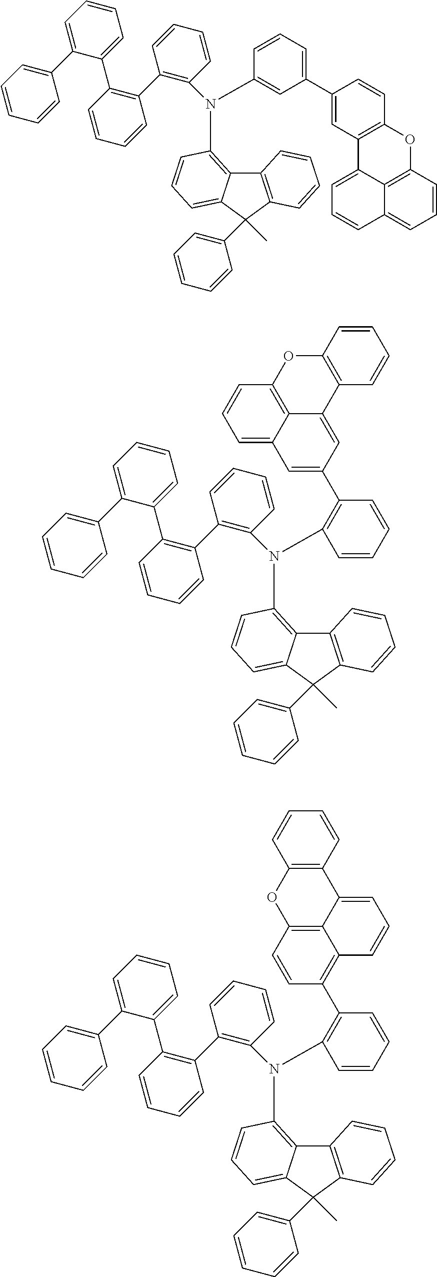 [SMILES: CC1(c2ccccc2)c2ccccc2-c2c(N(c3cccc(-c4ccc5c(c4)-c4cccc6cccc(c46)O5)c3)c3ccccc3-c3ccccc3-c3ccccc3-c3ccccc3)cccc21 ; CC1(c2ccccc2)c2ccccc2-c2c(N(c3ccccc3-c3cc4c5c(cccc5c3)Oc3ccccc3-4)c3ccccc3-c3ccccc3-c3ccccc3-c3ccccc3)cccc21 ; CC1(c2ccccc2)c2ccccc2-c2c(N(c3ccccc3-c3ccccc3-c3ccccc3-c3ccccc3)c3ccccc3-c3ccc4c5c(cccc35)-c3ccccc3O4)cccc21]

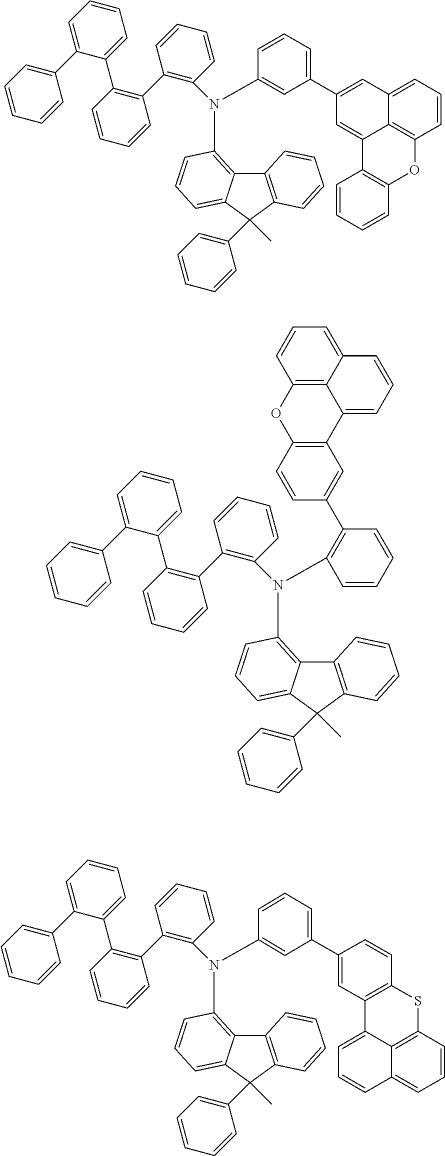 [SMILES: CC1(c2ccccc2)c2ccccc2-c2c(N(c3cccc(-c4cc5c6c(cccc6c4)Oc4ccccc4-5)c3)c3ccccc3-c3ccccc3-c3ccccc3-c3ccccc3)cccc21 ; CC1(c2ccccc2)c2ccccc2-c2c(N(c3ccccc3-c3ccc4c(c3)-c3cccc5cccc(c35)O4)c3ccccc3-c3ccccc3-c3ccccc3-c3ccccc3)cccc21 ; CC1(c2ccccc2)c2ccccc2-c2c(N(c3cccc(-c4ccc5c(c4)-c4cccc6cccc(c46)S5)c3)c3ccccc3-c3ccccc3-c3ccccc3-c3ccccc3)cccc21]

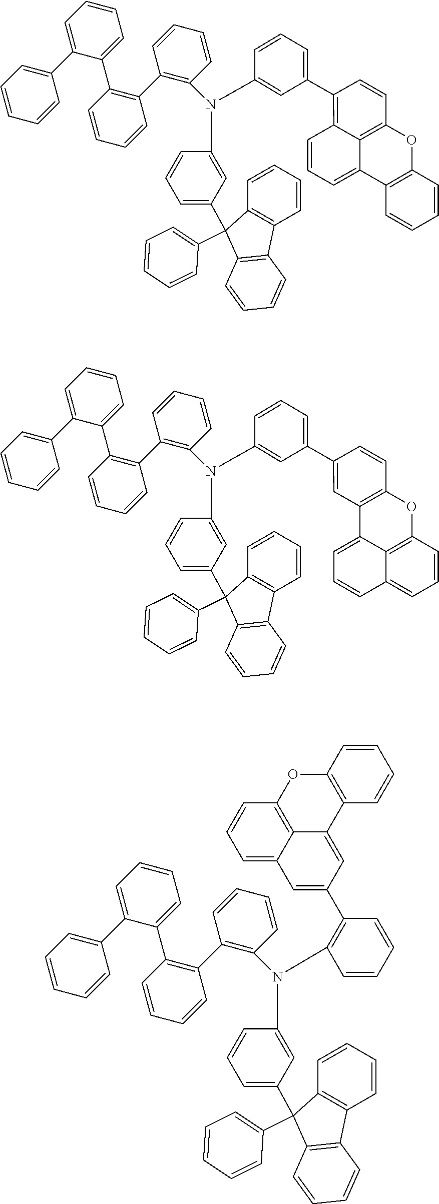 [SMILES: c1ccc(-c2ccccc2-c2ccccc2-c2ccccc2N(c2cccc(-c3ccc4c5c(cccc35)-c3ccccc3O4)c2)c2cccc(C3(c4ccccc4)c4ccccc4-c4ccccc43)c2)cc1 ; c1ccc(-c2ccccc2-c2ccccc2-c2ccccc2N(c2cccc(-c3ccc4c(c3)-c3cccc5cccc(c35)O4)c2)c2cccc(C3(c4ccccc4)c4ccccc4-c4ccccc43)c2)cc1 ; c1ccc(-c2ccccc2-c2ccccc2-c2ccccc2N(c2cccc(C3(c4ccccc4)c4ccccc4-c4ccccc43)c2)c2ccccc2-c2cc3c4c(cccc4c2)Oc2ccccc2-3)cc1]

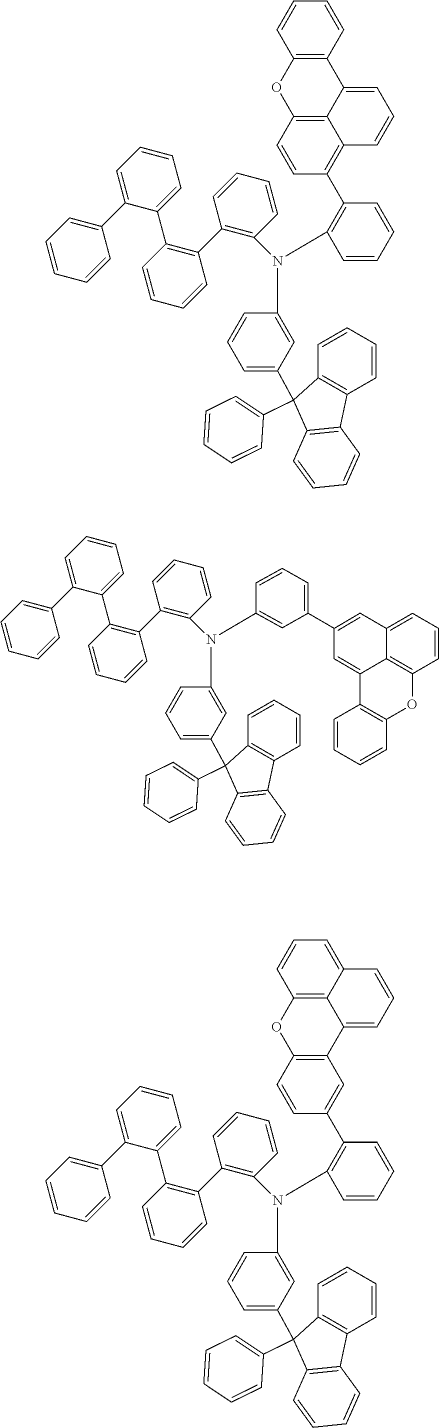 [SMILES: c1ccc(-c2ccccc2-c2ccccc2-c2ccccc2N(c2cccc(C3(c4ccccc4)c4ccccc4-c4ccccc43)c2)c2ccccc2-c2ccc3c4c(cccc24)-c2ccccc2O3)cc1 ; c1ccc(-c2ccccc2-c2ccccc2-c2ccccc2N(c2cccc(-c3cc4c5c(cccc5c3)Oc3ccccc3-4)c2)c2cccc(C3(c4ccccc4)c4ccccc4-c4ccccc43)c2)cc1 ; c1ccc(-c2ccccc2-c2ccccc2-c2ccccc2N(c2cccc(C3(c4ccccc4)c4ccccc4-c4ccccc43)c2)c2ccccc2-c2ccc3c(c2)-c2cccc4cccc(c24)O3)cc1]

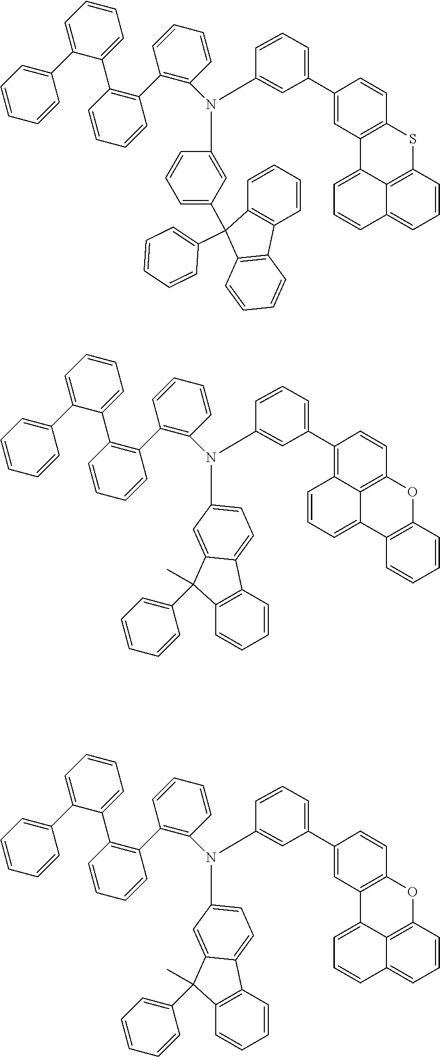 [SMILES: c1ccc(-c2ccccc2-c2ccccc2-c2ccccc2N(c2cccc(-c3ccc4c(c3)-c3cccc5cccc(c35)S4)c2)c2cccc(C3(c4ccccc4)c4ccccc4-c4ccccc43)c2)cc1 ; CC1(c2ccccc2)c2ccccc2-c2ccc(N(c3cccc(-c4ccc5c6c(cccc46)-c4ccccc4O5)c3)c3ccccc3-c3ccccc3-c3ccccc3-c3ccccc3)cc21 ; CC1(c2ccccc2)c2ccccc2-c2ccc(N(c3cccc(-c4ccc5c(c4)-c4cccc6cccc(c46)O5)c3)c3ccccc3-c3ccccc3-c3ccccc3-c3ccccc3)cc21]

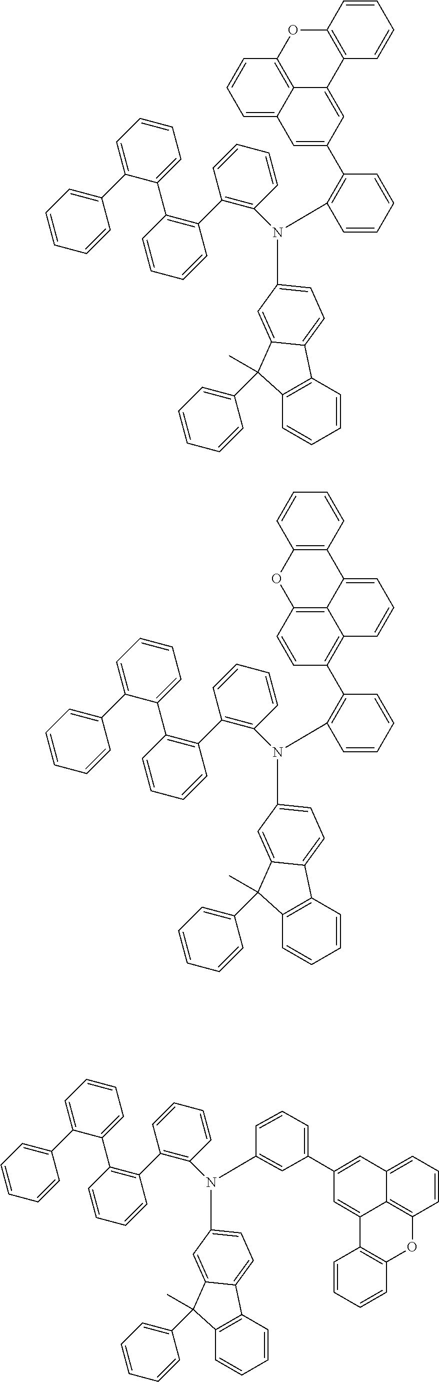 [SMILES: CC1(c2ccccc2)c2ccccc2-c2ccc(N(c3ccccc3-c3cc4c5c(cccc5c3)Oc3ccccc3-4)c3ccccc3-c3ccccc3-c3ccccc3-c3ccccc3)cc21 ; CC1(c2ccccc2)c2ccccc2-c2ccc(N(c3ccccc3-c3ccccc3-c3ccccc3-c3ccccc3)c3ccccc3-c3ccc4c5c(cccc35)-c3ccccc3O4)cc21 ; CC1(c2ccccc2)c2ccccc2-c2ccc(N(c3cccc(-c4cc5c6c(cccc6c4)Oc4ccccc4-5)c3)c3ccccc3-c3ccccc3-c3ccccc3-c3ccccc3)cc21]

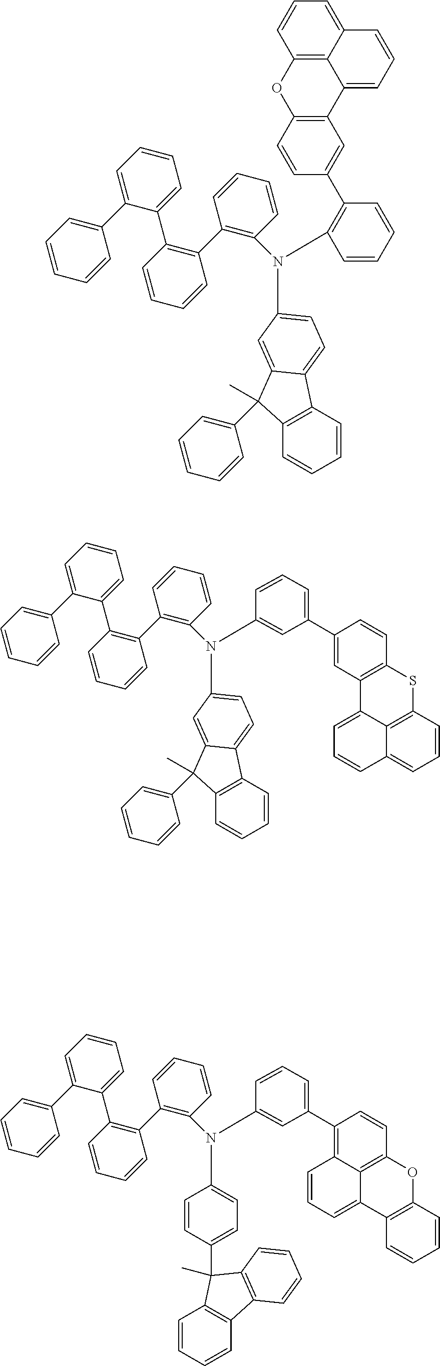 [SMILES: CC1(c2ccccc2)c2ccccc2-c2ccc(N(c3ccccc3-c3ccc4c(c3)-c3cccc5cccc(c35)O4)c3ccccc3-c3ccccc3-c3ccccc3-c3ccccc3)cc21 ; CC1(c2ccccc2)c2ccccc2-c2ccc(N(c3cccc(-c4ccc5c(c4)-c4cccc6cccc(c46)S5)c3)c3ccccc3-c3ccccc3-c3ccccc3-c3ccccc3)cc21 ; CC1(c2ccc(N(c3cccc(-c4ccc5c6c(cccc46)-c4ccccc4O5)c3)c3ccccc3-c3ccccc3-c3ccccc3-c3ccccc3)cc2)c2ccccc2-c2ccccc21]

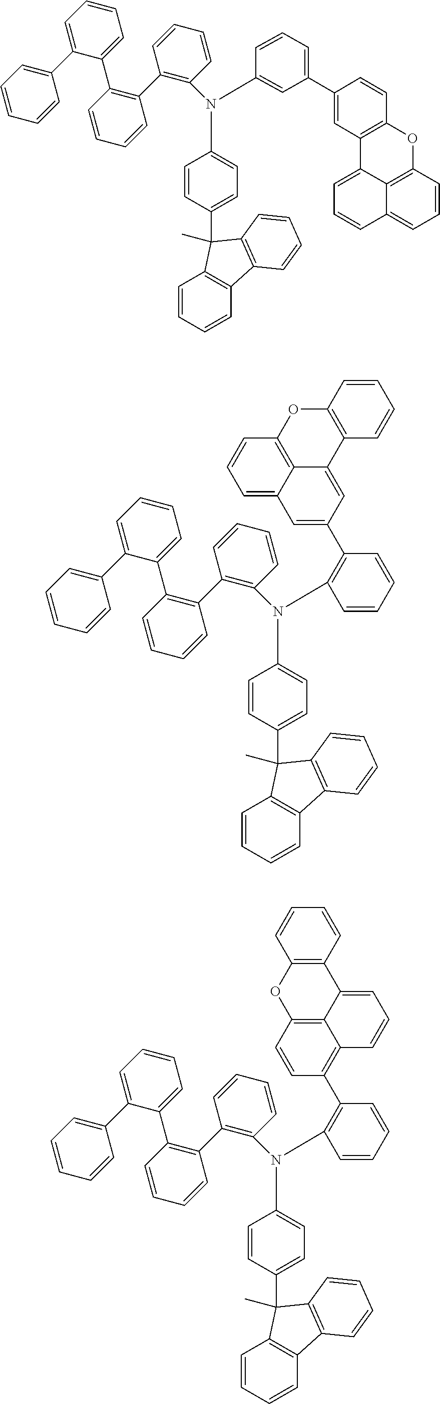 [SMILES: CC1(c2ccc(N(c3cccc(-c4ccc5c(c4)-c4cccc6cccc(c46)O5)c3)c3ccccc3-c3ccccc3-c3ccccc3-c3ccccc3)cc2)c2ccccc2-c2ccccc21 ; CC1(c2ccc(N(c3ccccc3-c3cc4c5c(cccc5c3)Oc3ccccc3-4)c3ccccc3-c3ccccc3-c3ccccc3-c3ccccc3)cc2)c2ccccc2-c2ccccc21 ; CC1(c2ccc(N(c3ccccc3-c3ccccc3-c3ccccc3-c3ccccc3)c3ccccc3-c3ccc4c5c(cccc35)-c3ccccc3O4)cc2)c2ccccc2-c2ccccc21]

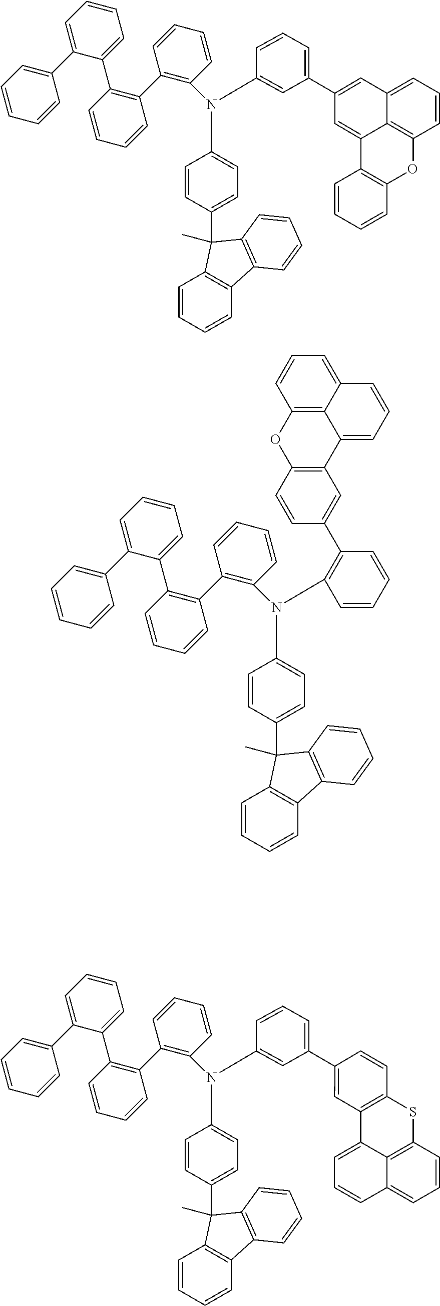 [SMILES: CC1(c2ccc(N(c3cccc(-c4cc5c6c(cccc6c4)Oc4ccccc4-5)c3)c3ccccc3-c3ccccc3-c3ccccc3-c3ccccc3)cc2)c2ccccc2-c2ccccc21 ; CC1(c2ccc(N(c3ccccc3-c3ccc4c(c3)-c3cccc5cccc(c35)O4)c3ccccc3-c3ccccc3-c3ccccc3-c3ccccc3)cc2)c2ccccc2-c2ccccc21 ; CC1(c2ccc(N(c3cccc(-c4ccc5c(c4)-c4cccc6cccc(c46)S5)c3)c3ccccc3-c3ccccc3-c3ccccc3-c3ccccc3)cc2)c2ccccc2-c2ccccc21]

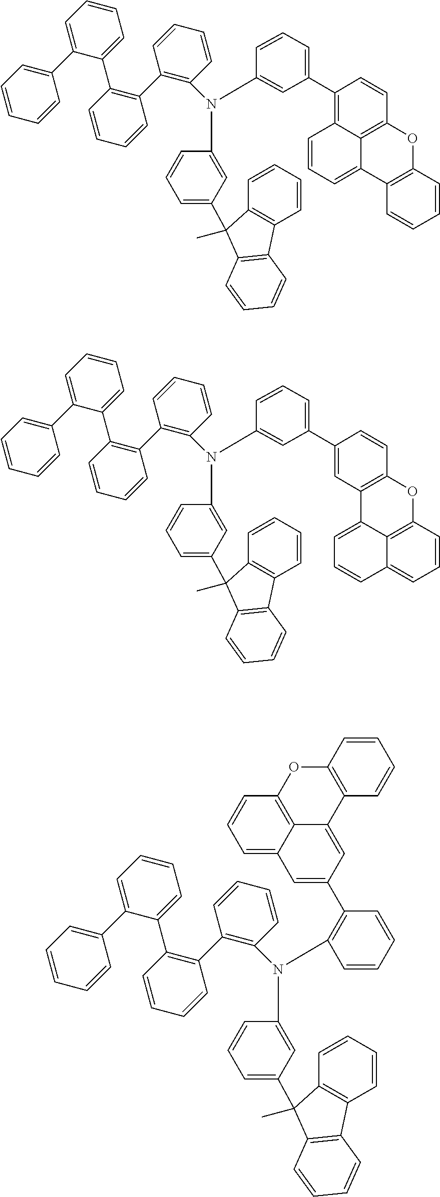 [SMILES: CC1(c2cccc(N(c3cccc(-c4ccc5c6c(cccc46)-c4ccccc4O5)c3)c3ccccc3-c3ccccc3-c3ccccc3-c3ccccc3)c2)c2ccccc2-c2ccccc21 ; CC1(c2cccc(N(c3cccc(-c4ccc5c(c4)-c4cccc6cccc(c46)O5)c3)c3ccccc3-c3ccccc3-c3ccccc3-c3ccccc3)c2)c2ccccc2-c2ccccc21 ; CC1(c2cccc(N(c3ccccc3-c3cc4c5c(cccc5c3)Oc3ccccc3-4)c3ccccc3-c3ccccc3-c3ccccc3-c3ccccc3)c2)c2ccccc2-c2ccccc21]

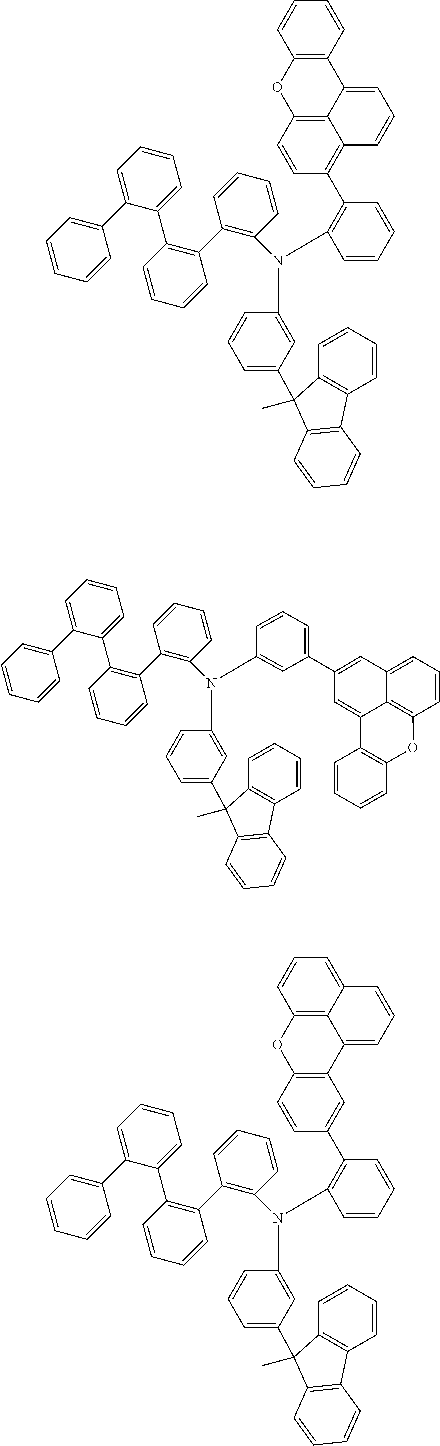 [SMILES: CC1(c2cccc(N(c3ccccc3-c3ccccc3-c3ccccc3-c3ccccc3)c3ccccc3-c3ccc4c5c(cccc35)-c3ccccc3O4)c2)c2ccccc2-c2ccccc21 ; CC1(c2cccc(N(c3cccc(-c4cc5c6c(cccc6c4)Oc4ccccc4-5)c3)c3ccccc3-c3ccccc3-c3ccccc3-c3ccccc3)c2)c2ccccc2-c2ccccc21 ; CC1(c2cccc(N(c3ccccc3-c3ccc4c(c3)-c3cccc5cccc(c35)O4)c3ccccc3-c3ccccc3-c3ccccc3-c3ccccc3)c2)c2ccccc2-c2ccccc21]

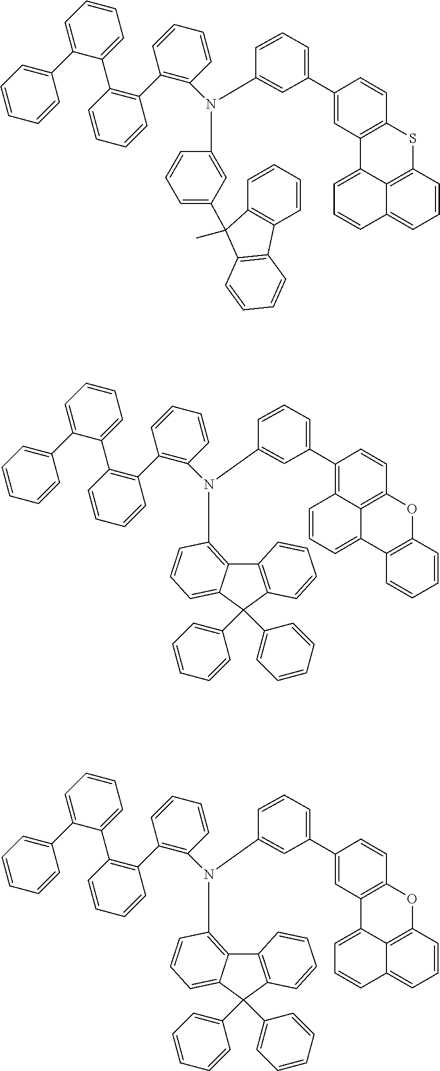 [SMILES: CC1(c2cccc(N(c3cccc(-c4ccc5c(c4)-c4cccc6cccc(c46)S5)c3)c3ccccc3-c3ccccc3-c3ccccc3-c3ccccc3)c2)c2ccccc2-c2ccccc21 ; c1ccc(-c2ccccc2-c2ccccc2-c2ccccc2N(c2cccc(-c3ccc4c5c(cccc35)-c3ccccc3O4)c2)c2cccc3c2-c2ccccc2C3(c2ccccc2)c2ccccc2)cc1 ; c1ccc(-c2ccccc2-c2ccccc2-c2ccccc2N(c2cccc(-c3ccc4c(c3)-c3cccc5cccc(c35)O4)c2)c2cccc3c2-c2ccccc2C3(c2ccccc2)c2ccccc2)cc1]

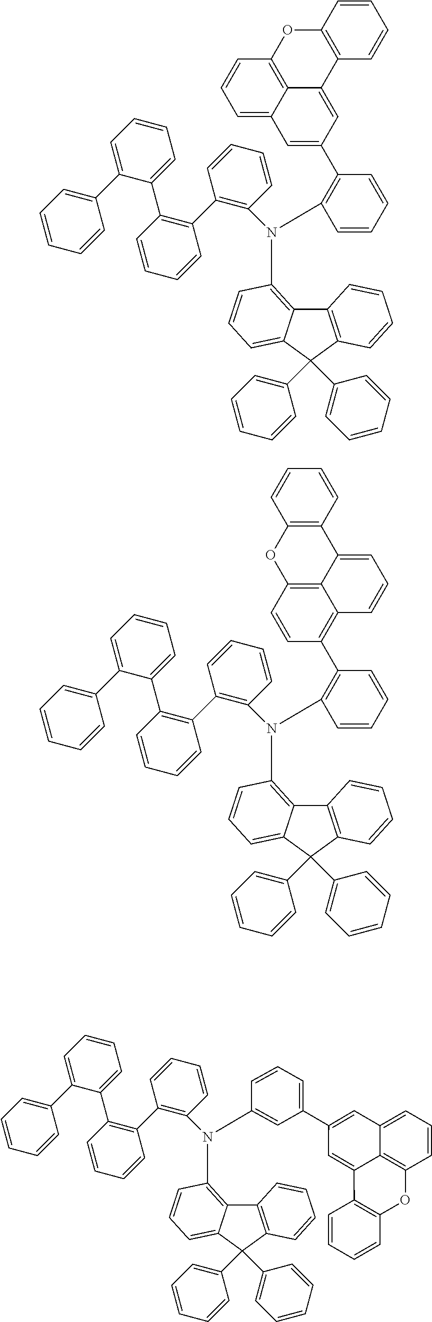

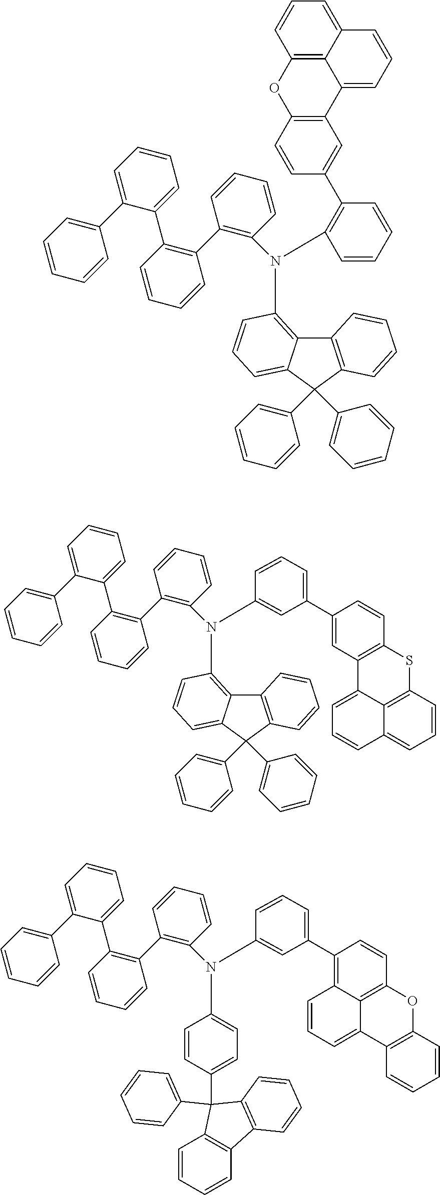 [SMILES: c1ccc(-c2ccccc2-c2ccccc2-c2ccccc2N(c2ccccc2-c2ccc3c(c2)-c2cccc4cccc(c24)O3)c2cccc3c2-c2ccccc2C3(c2ccccc2)c2ccccc2)cc1 ; c1ccc(-c2ccccc2-c2ccccc2-c2ccccc2N(c2cccc(-c3ccc4c(c3)-c3cccc5cccc(c35)S4)c2)c2cccc3c2-c2ccccc2C3(c2ccccc2)c2ccccc2)cc1 ; c1ccc(-c2ccccc2-c2ccccc2-c2ccccc2N(c2ccc(C3(c4ccccc4)c4ccccc4-c4ccccc43)cc2)c2cccc(-c3ccc4c5c(cccc35)-c3ccccc3O4)c2)cc1]

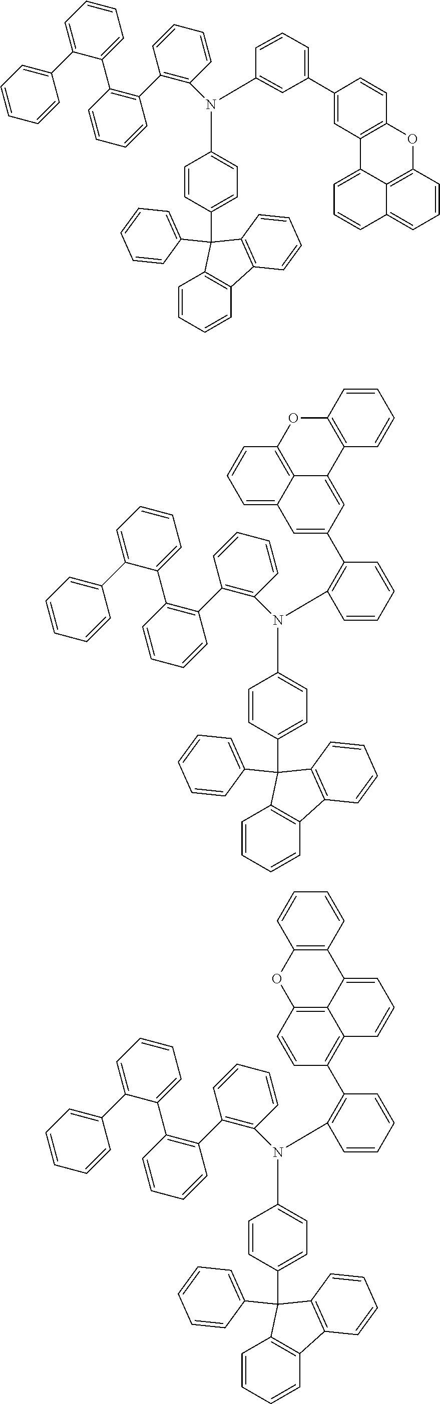

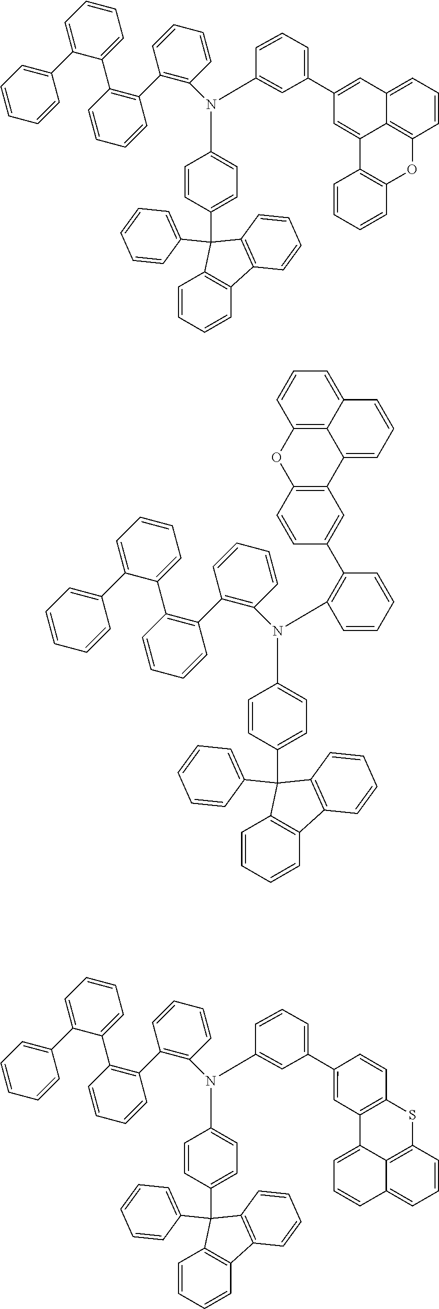 [SMILES: c1ccc(-c2ccccc2-c2ccccc2-c2ccccc2N(c2ccc(C3(c4ccccc4)c4ccccc4-c4ccccc43)cc2)c2cccc(-c3cc4c5c(cccc5c3)Oc3ccccc3-4)c2)cc1 ; c1ccc(-c2ccccc2-c2ccccc2-c2ccccc2N(c2ccc(C3(c4ccccc4)c4ccccc4-c4ccccc43)cc2)c2ccccc2-c2ccc3c(c2)-c2cccc4cccc(c24)O3)cc1 ; c1ccc(-c2ccccc2-c2ccccc2-c2ccccc2N(c2ccc(C3(c4ccccc4)c4ccccc4-c4ccccc43)cc2)c2cccc(-c3ccc4c(c3)-c3cccc5cccc(c35)S4)c2)cc1]

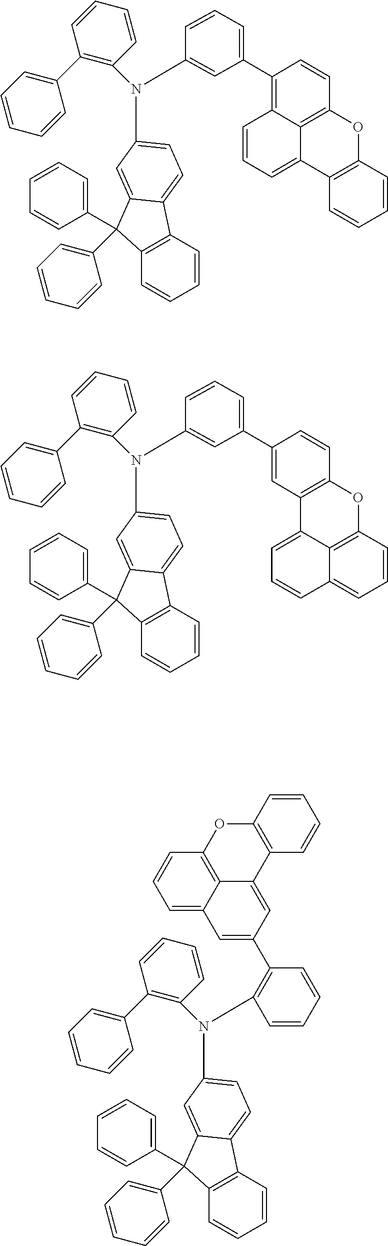 [SMILES: c1ccc(-c2ccccc2N(c2cccc(-c3ccc4c5c(cccc35)-c3ccccc3O4)c2)c2ccc3c(c2)C(c2ccccc2)(c2ccccc2)c2ccccc2-3)cc1 ; c1ccc(-c2ccccc2N(c2cccc(-c3ccc4c(c3)-c3cccc5cccc(c35)O4)c2)c2ccc3c(c2)C(c2ccccc2)(c2ccccc2)c2ccccc2-3)cc1 ; c1ccc(-c2ccccc2N(c2ccc3c(c2)C(c2ccccc2)(c2ccccc2)c2ccccc2-3)c2ccccc2-c2cc3c4c(cccc4c2)Oc2ccccc2-3)cc1]

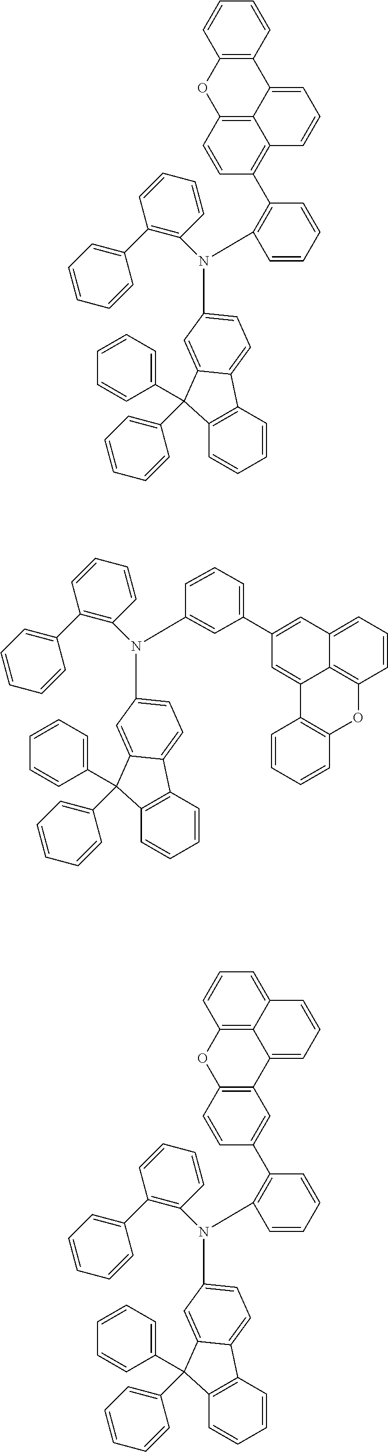 [SMILES: c1ccc(-c2ccccc2N(c2ccc3c(c2)C(c2ccccc2)(c2ccccc2)c2ccccc2-3)c2ccccc2-c2ccc3c4c(cccc24)-c2ccccc2O3)cc1 ; c1ccc(-c2ccccc2N(c2cccc(-c3cc4c5c(cccc5c3)Oc3ccccc3-4)c2)c2ccc3c(c2)C(c2ccccc2)(c2ccccc2)c2ccccc2-3)cc1 ; c1ccc(-c2ccccc2N(c2ccc3c(c2)C(c2ccccc2)(c2ccccc2)c2ccccc2-3)c2ccccc2-c2ccc3c(c2)-c2cccc4cccc(c24)O3)cc1]

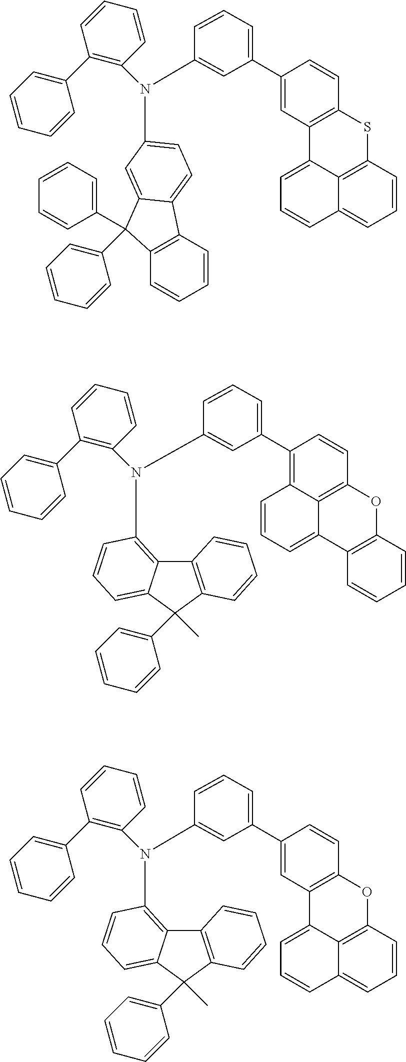 [SMILES: c1ccc(-c2ccccc2N(c2cccc(-c3ccc4c(c3)-c3cccc5cccc(c35)S4)c2)c2ccc3c(c2)C(c2ccccc2)(c2ccccc2)c2ccccc2-3)cc1 ; CC1(c2ccccc2)c2ccccc2-c2c(N(c3cccc(-c4ccc5c6c(cccc46)-c4ccccc4O5)c3)c3ccccc3-c3ccccc3)cccc21 ; CC1(c2ccccc2)c2ccccc2-c2c(N(c3cccc(-c4ccc5c(c4)-c4cccc6cccc(c46)O5)c3)c3ccccc3-c3ccccc3)cccc21]

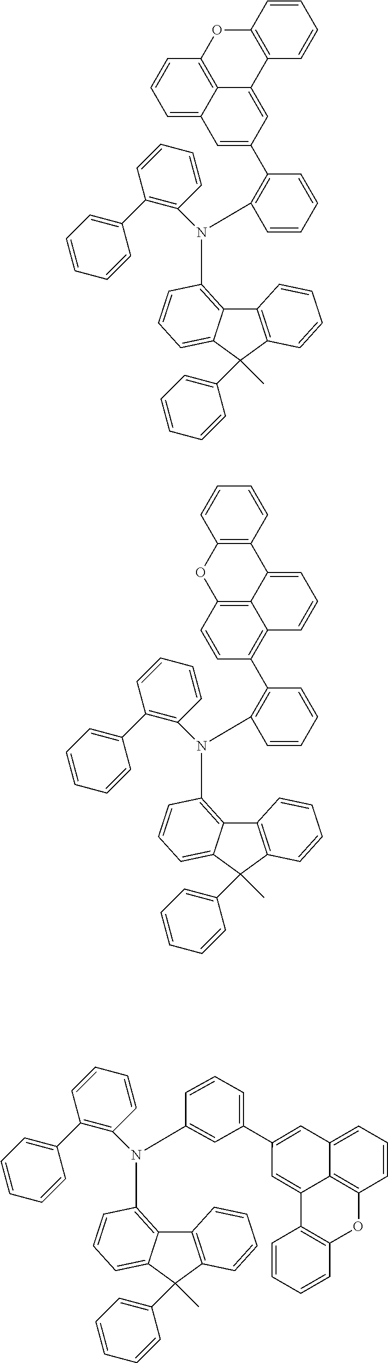 [SMILES: CC1(c2ccccc2)c2ccccc2-c2c(N(c3ccccc3-c3ccccc3)c3ccccc3-c3cc4c5c(cccc5c3)Oc3ccccc3-4)cccc21 ; CC1(c2ccccc2)c2ccccc2-c2c(N(c3ccccc3-c3ccccc3)c3ccccc3-c3ccc4c5c(cccc35)-c3ccccc3O4)cccc21 ; CC1(c2ccccc2)c2ccccc2-c2c(N(c3cccc(-c4cc5c6c(cccc6c4)Oc4ccccc4-5)c3)c3ccccc3-c3ccccc3)cccc21]

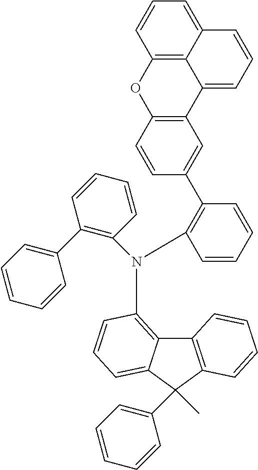 [SMILES: CC1(c2ccccc2)c2ccccc2-c2c(N(c3ccccc3-c3ccccc3)c3ccccc3-c3ccc4c(c3)-c3cccc5cccc(c35)O4)cccc21]

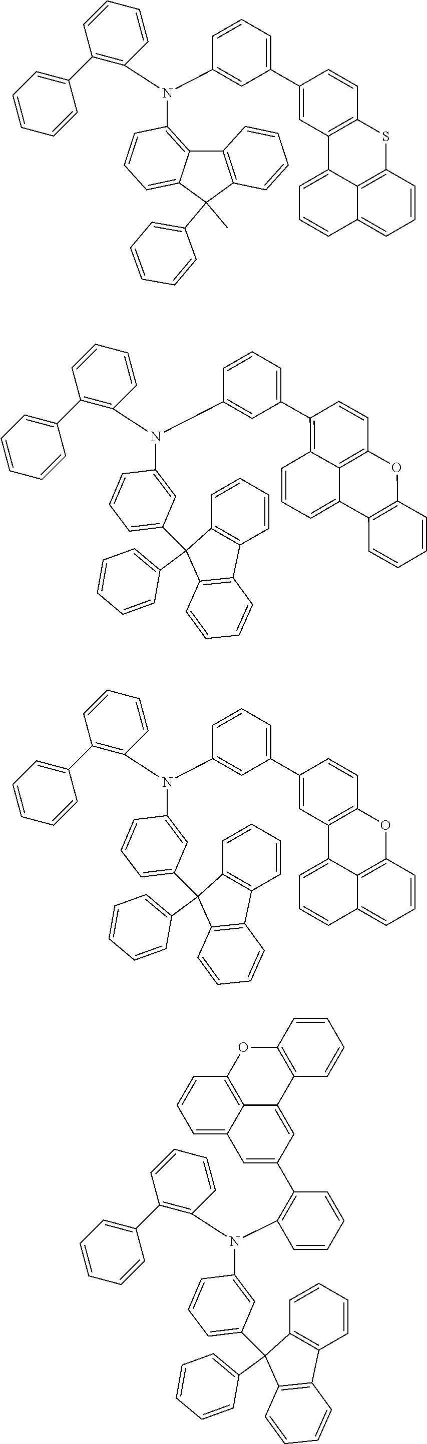 [SMILES: CC1(c2ccccc2)c2ccccc2-c2c(N(c3cccc(-c4ccc5c(c4)-c4cccc6cccc(c46)S5)c3)c3ccccc3-c3ccccc3)cccc21 ; c1ccc(-c2ccccc2N(c2cccc(-c3ccc4c5c(cccc35)-c3ccccc3O4)c2)c2cccc(C3(c4ccccc4)c4ccccc4-c4ccccc43)c2)cc1 ; c1ccc(-c2ccccc2N(c2cccc(-c3ccc4c(c3)-c3cccc5cccc(c35)O4)c2)c2cccc(C3(c4ccccc4)c4ccccc4-c4ccccc43)c2)cc1 ; c1ccc(-c2ccccc2N(c2cccc(C3(c4ccccc4)c4ccccc4-c4ccccc43)c2)c2ccccc2-c2cc3c4c(cccc4c2)Oc2ccccc2-3)cc1]

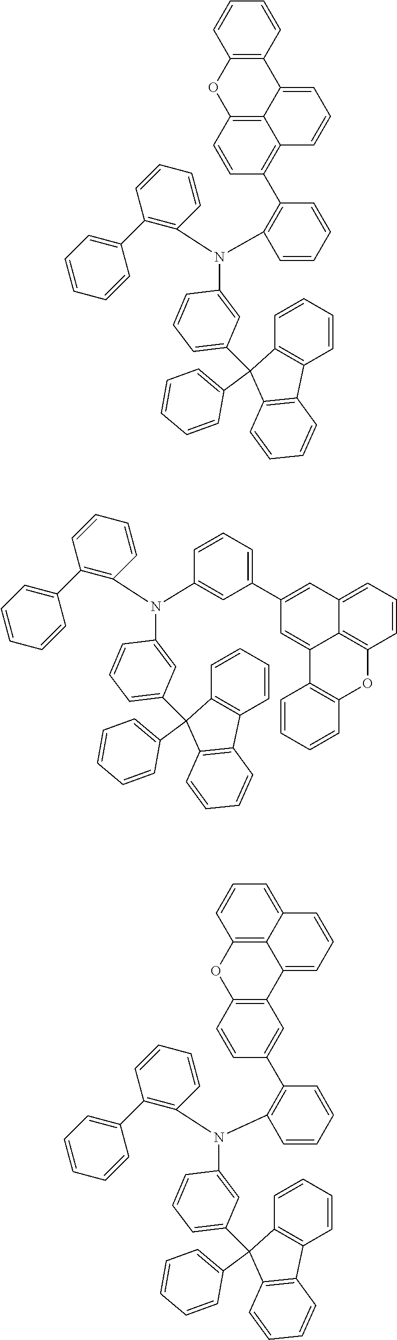 [SMILES: c1ccc(-c2ccccc2N(c2cccc(C3(c4ccccc4)c4ccccc4-c4ccccc43)c2)c2ccccc2-c2ccc3c4c(cccc24)-c2ccccc2O3)cc1 ; c1ccc(-c2ccccc2N(c2cccc(-c3cc4c5c(cccc5c3)Oc3ccccc3-4)c2)c2cccc(C3(c4ccccc4)c4ccccc4-c4ccccc43)c2)cc1 ; c1ccc(-c2ccccc2N(c2cccc(C3(c4ccccc4)c4ccccc4-c4ccccc43)c2)c2ccccc2-c2ccc3c(c2)-c2cccc4cccc(c24)O3)cc1]

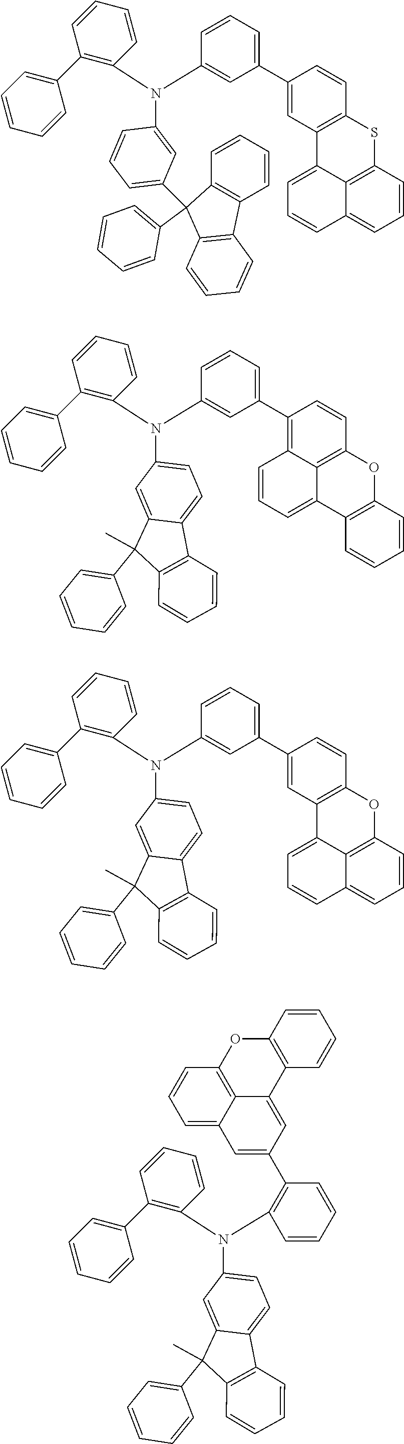

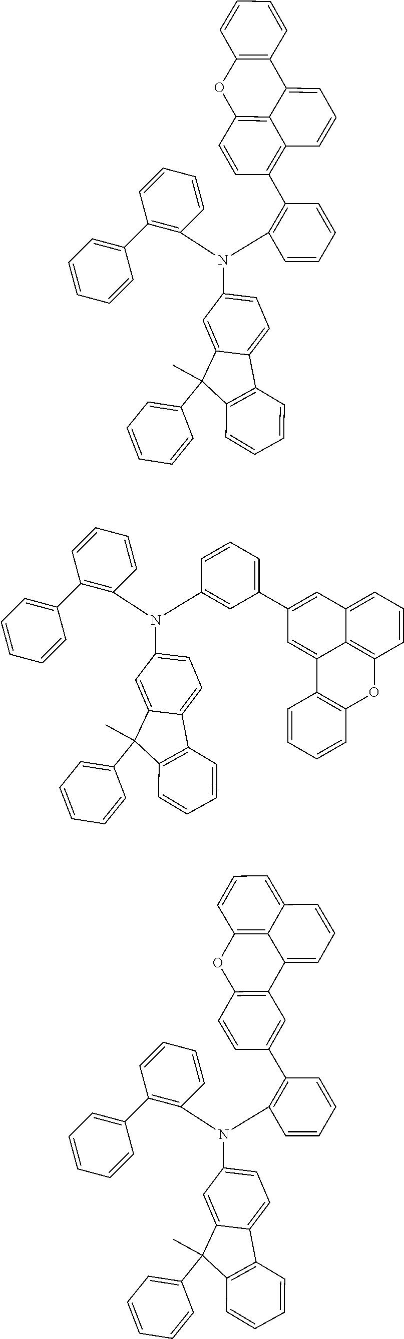 [SMILES: CC1(c2ccccc2)c2ccccc2-c2ccc(N(c3ccccc3-c3ccccc3)c3ccccc3-c3ccc4c5c(cccc35)-c3ccccc3O4)cc21 ; CC1(c2ccccc2)c2ccccc2-c2ccc(N(c3cccc(-c4cc5c6c(cccc6c4)Oc4ccccc4-5)c3)c3ccccc3-c3ccccc3)cc21 ; CC1(c2ccccc2)c2ccccc2-c2ccc(N(c3ccccc3-c3ccccc3)c3ccccc3-c3ccc4c(c3)-c3cccc5cccc(c35)O4)cc21]

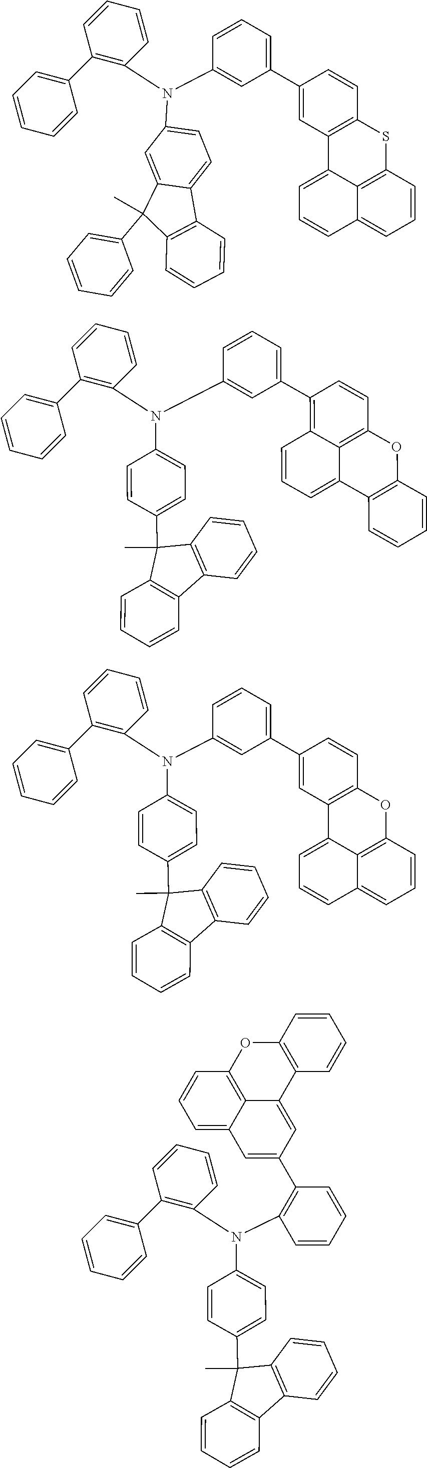

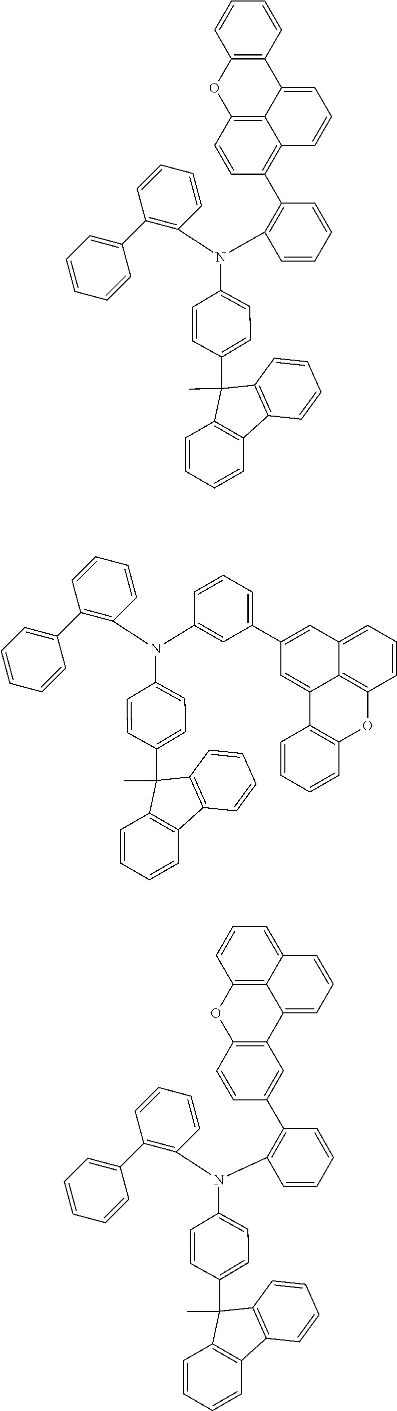 [SMILES: CC1(c2ccc(N(c3ccccc3-c3ccccc3)c3ccccc3-c3ccc4c5c(cccc35)-c3ccccc3O4)cc2)c2ccccc2-c2ccccc21 ; CC1(c2ccc(N(c3cccc(-c4cc5c6c(cccc6c4)Oc4ccccc4-5)c3)c3ccccc3-c3ccccc3)cc2)c2ccccc2-c2ccccc21 ; CC1(c2ccc(N(c3ccccc3-c3ccccc3)c3ccccc3-c3ccc4c(c3)-c3cccc5cccc(c35)O4)cc2)c2ccccc2-c2ccccc21]

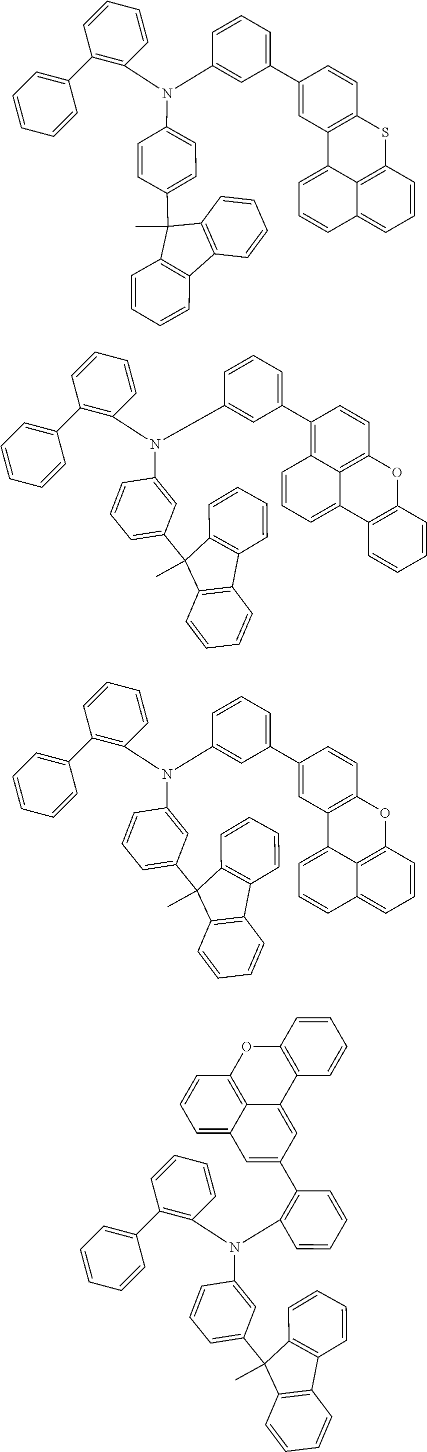 [SMILES: CC1(c2ccc(N(c3cccc(-c4ccc5c(c4)-c4cccc6cccc(c46)S5)c3)c3ccccc3-c3ccccc3)cc2)c2ccccc2-c2ccccc21 ; CC1(c2cccc(N(c3cccc(-c4ccc5c6c(cccc46)-c4ccccc4O5)c3)c3ccccc3-c3ccccc3)c2)c2ccccc2-c2ccccc21 ; CC1(c2cccc(N(c3cccc(-c4ccc5c(c4)-c4cccc6cccc(c46)O5)c3)c3ccccc3-c3ccccc3)c2)c2ccccc2-c2ccccc21 ; CC1(c2cccc(N(c3ccccc3-c3ccccc3)c3ccccc3-c3cc4c5c(cccc5c3)Oc3ccccc3-4)c2)c2ccccc2-c2ccccc21]

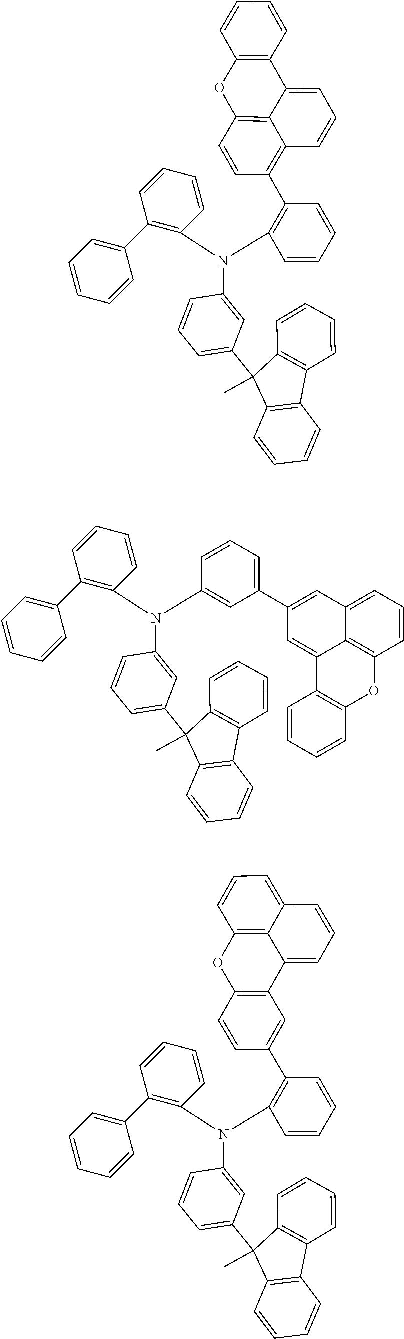 [SMILES: CC1(c2cccc(N(c3ccccc3-c3ccccc3)c3ccccc3-c3ccc4c5c(cccc35)-c3ccccc3O4)c2)c2ccccc2-c2ccccc21 ; CC1(c2cccc(N(c3cccc(-c4cc5c6c(cccc6c4)Oc4ccccc4-5)c3)c3ccccc3-c3ccccc3)c2)c2ccccc2-c2ccccc21 ; CC1(c2cccc(N(c3ccccc3-c3ccccc3)c3ccccc3-c3ccc4c(c3)-c3cccc5cccc(c35)O4)c2)c2ccccc2-c2ccccc21]

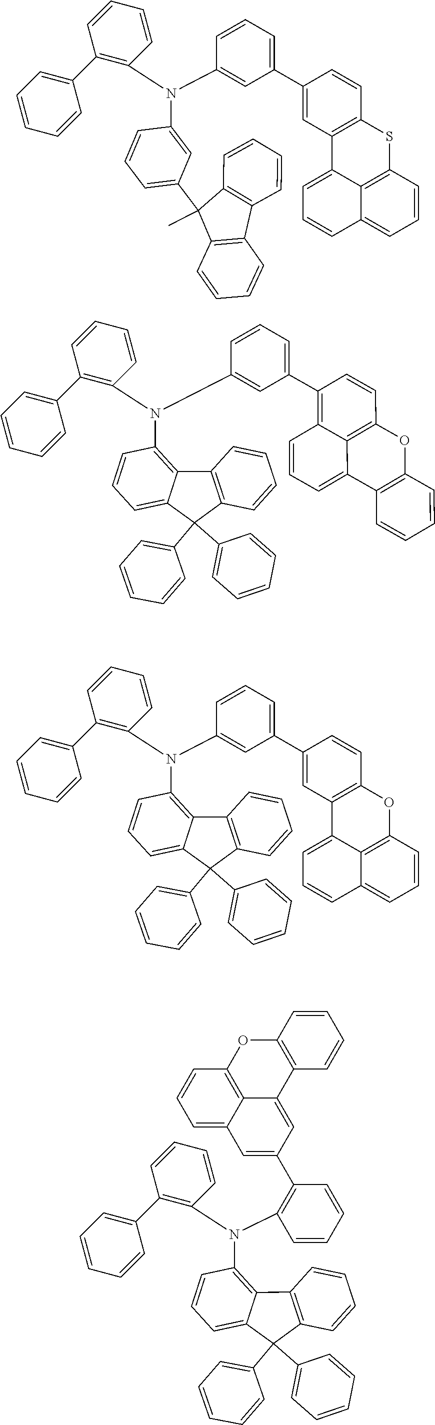

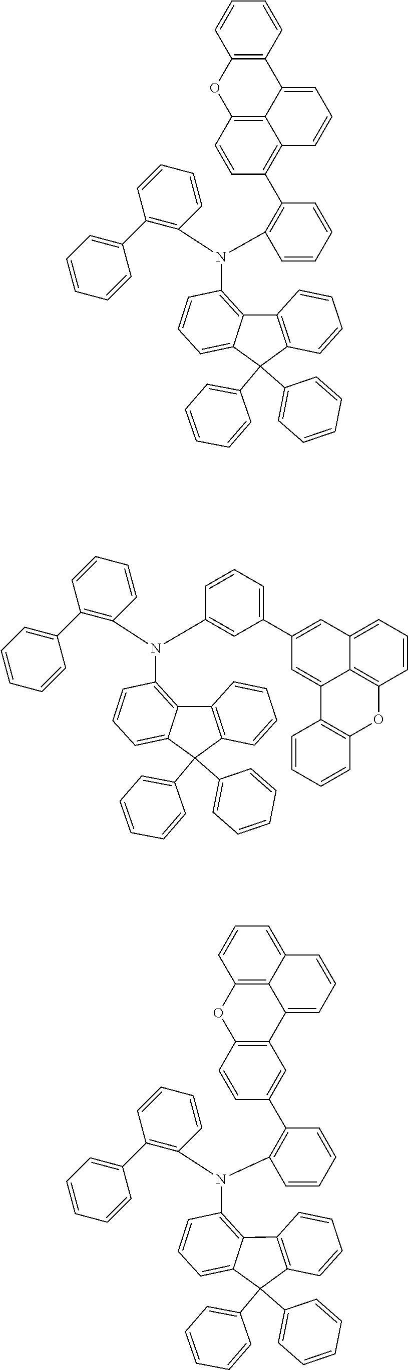 [SMILES: c1ccc(-c2ccccc2N(c2ccccc2-c2ccc3c4c(cccc24)-c2ccccc2O3)c2cccc3c2-c2ccccc2C3(c2ccccc2)c2ccccc2)cc1 ; c1ccc(-c2ccccc2N(c2cccc(-c3cc4c5c(cccc5c3)Oc3ccccc3-4)c2)c2cccc3c2-c2ccccc2C3(c2ccccc2)c2ccccc2)cc1 ; c1ccc(-c2ccccc2N(c2ccccc2-c2ccc3c(c2)-c2cccc4cccc(c24)O3)c2cccc3c2-c2ccccc2C3(c2ccccc2)c2ccccc2)cc1]

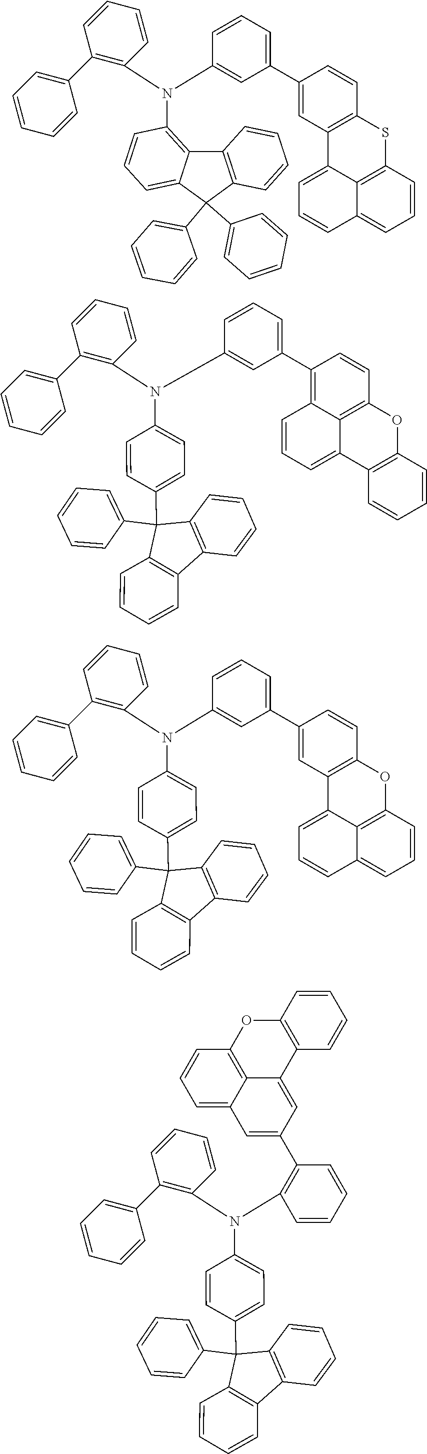

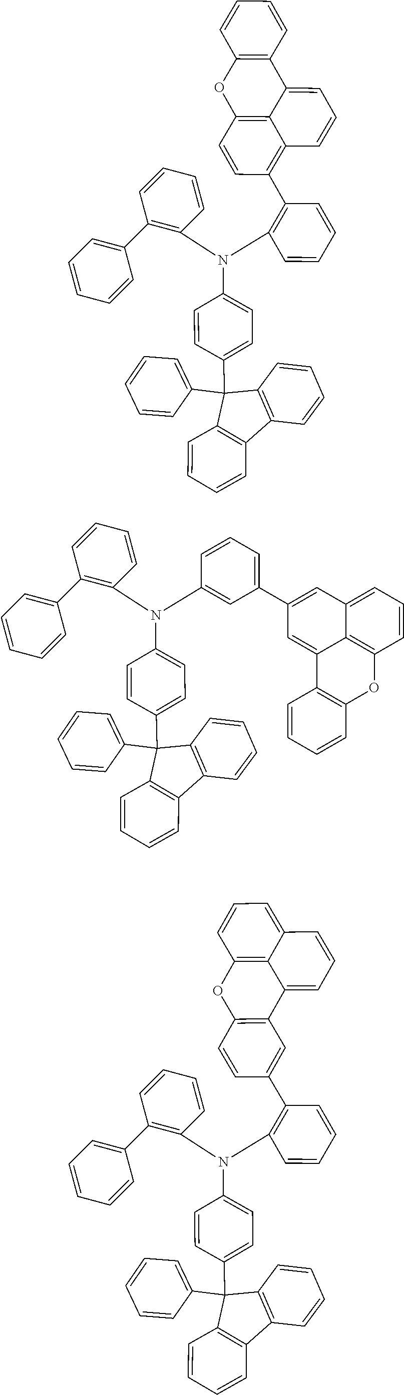 [SMILES: c1ccc(-c2ccccc2N(c2ccc(C3(c4ccccc4)c4ccccc4-c4ccccc43)cc2)c2ccccc2-c2ccc3c4c(cccc24)-c2ccccc2O3)cc1 ; c1ccc(-c2ccccc2N(c2ccc(C3(c4ccccc4)c4ccccc4-c4ccccc43)cc2)c2cccc(-c3cc4c5c(cccc5c3)Oc3ccccc3-4)c2)cc1 ; c1ccc(-c2ccccc2N(c2ccc(C3(c4ccccc4)c4ccccc4-c4ccccc43)cc2)c2ccccc2-c2ccc3c(c2)-c2cccc4cccc(c24)O3)cc1]

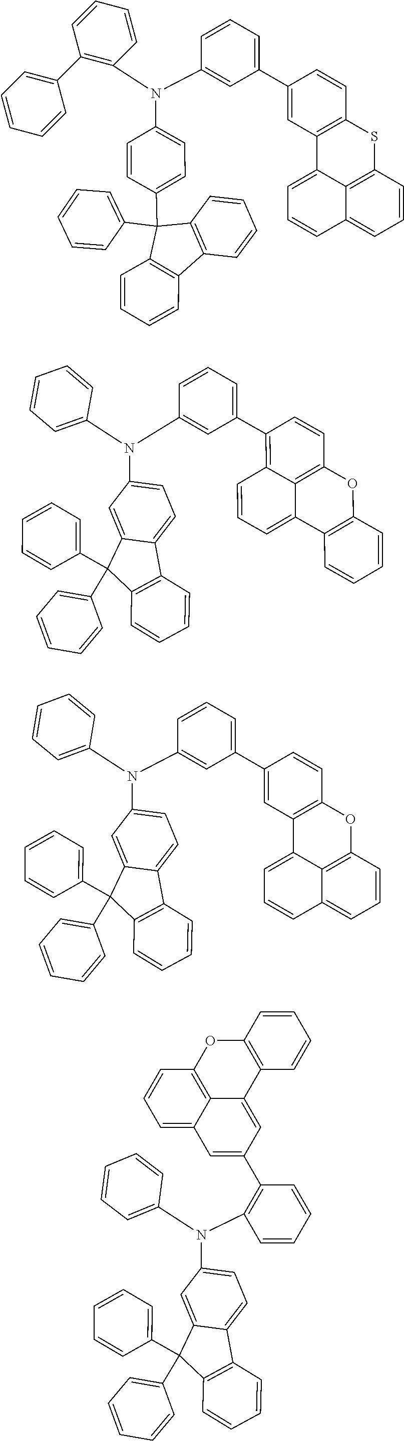

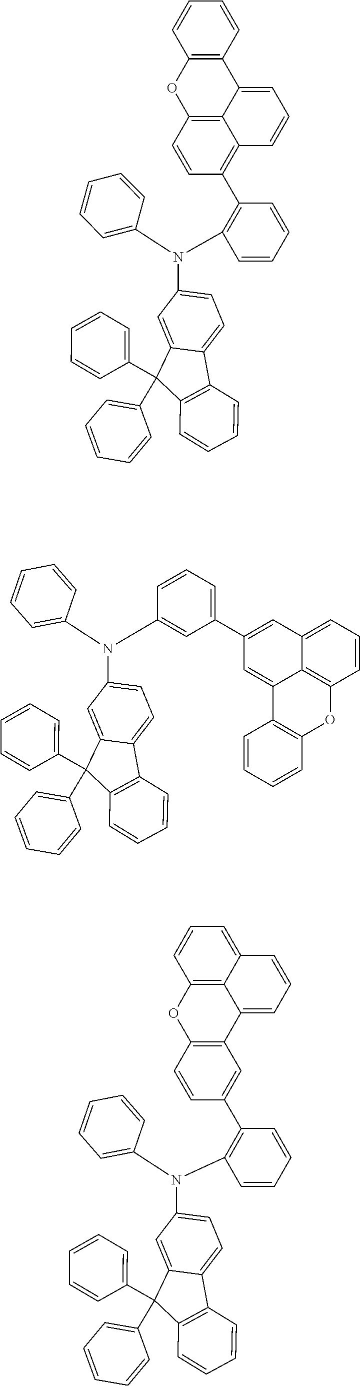 [SMILES: c1ccc(N(c2ccc3c(c2)C(c2ccccc2)(c2ccccc2)c2ccccc2-3)c2ccccc2-c2ccc3c4c(cccc24)-c2ccccc2O3)cc1 ; c1ccc(N(c2cccc(-c3cc4c5c(cccc5c3)Oc3ccccc3-4)c2)c2ccc3c(c2)C(c2ccccc2)(c2ccccc2)c2ccccc2-3)cc1 ; c1ccc(N(c2ccc3c(c2)C(c2ccccc2)(c2ccccc2)c2ccccc2-3)c2ccccc2-c2ccc3c(c2)-c2cccc4cccc(c24)O3)cc1]

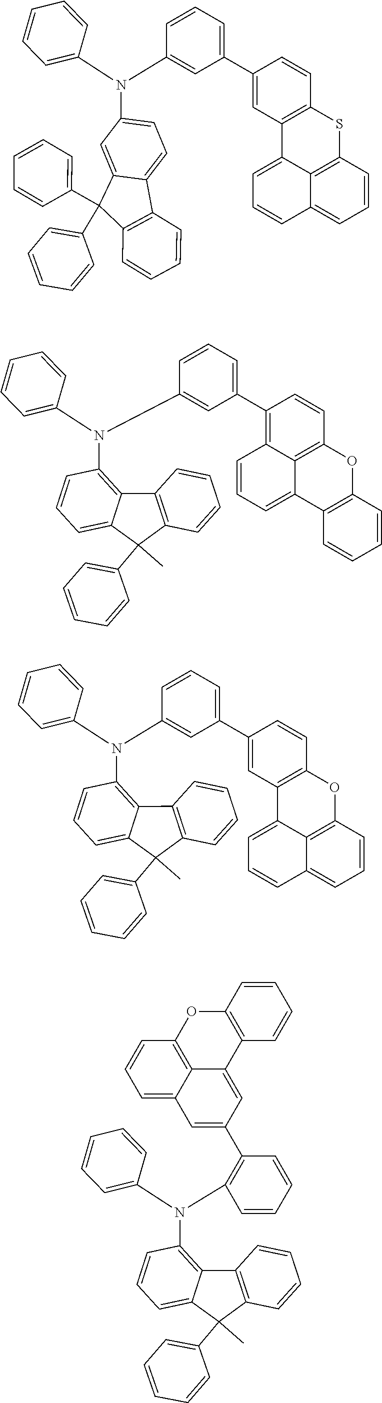 [SMILES: c1ccc(N(c2cccc(-c3ccc4c(c3)-c3cccc5cccc(c35)S4)c2)c2ccc3c(c2)C(c2ccccc2)(c2ccccc2)c2ccccc2-3)cc1 ; CC1(c2ccccc2)c2ccccc2-c2c(N(c3ccccc3)c3cccc(-c4ccc5c6c(cccc46)-c4ccccc4O5)c3)cccc21 ; CC1(c2ccccc2)c2ccccc2-c2c(N(c3ccccc3)c3cccc(-c4ccc5c(c4)-c4cccc6cccc(c46)O5)c3)cccc21 ; CC1(c2ccccc2)c2ccccc2-c2c(N(c3ccccc3)c3ccccc3-c3cc4c5c(cccc5c3)Oc3ccccc3-4)cccc21]

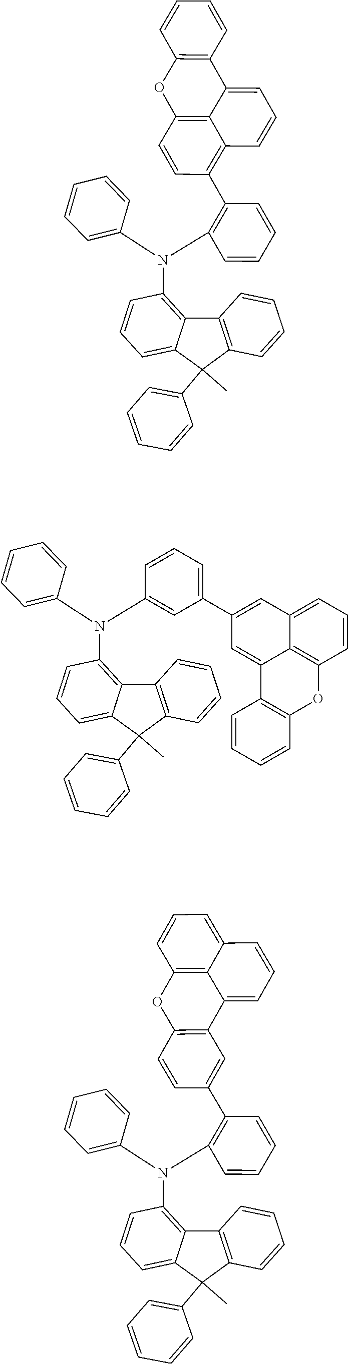 [SMILES: CC1(c2ccccc2)c2ccccc2-c2c(N(c3ccccc3)c3ccccc3-c3ccc4c5c(cccc35)-c3ccccc3O4)cccc21 ; CC1(c2ccccc2)c2ccccc2-c2c(N(c3ccccc3)c3cccc(-c4cc5c6c(cccc6c4)Oc4ccccc4-5)c3)cccc21 ; CC1(c2ccccc2)c2ccccc2-c2c(N(c3ccccc3)c3ccccc3-c3ccc4c(c3)-c3cccc5cccc(c35)O4)cccc21]

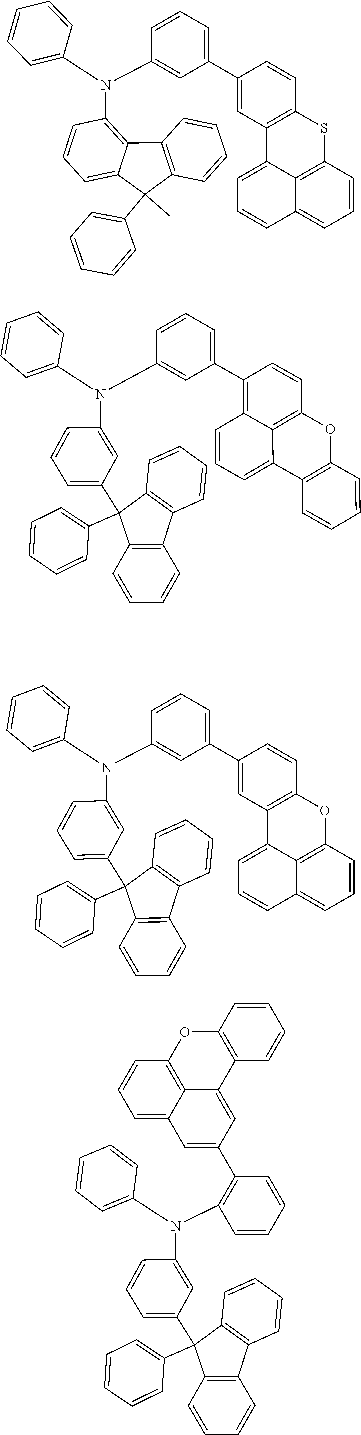 [SMILES: CC1(c2ccccc2)c2ccccc2-c2c(N(c3ccccc3)c3cccc(-c4ccc5c(c4)-c4cccc6cccc(c46)S5)c3)cccc21 ; c1ccc(N(c2cccc(-c3ccc4c5c(cccc35)-c3ccccc3O4)c2)c2cccc(C3(c4ccccc4)c4ccccc4-c4ccccc43)c2)cc1 ; c1ccc(N(c2cccc(-c3ccc4c(c3)-c3cccc5cccc(c35)O4)c2)c2cccc(C3(c4ccccc4)c4ccccc4-c4ccccc43)c2)cc1 ; c1ccc(N(c2cccc(C3(c4ccccc4)c4ccccc4-c4ccccc43)c2)c2ccccc2-c2cc3c4c(cccc4c2)Oc2ccccc2-3)cc1]

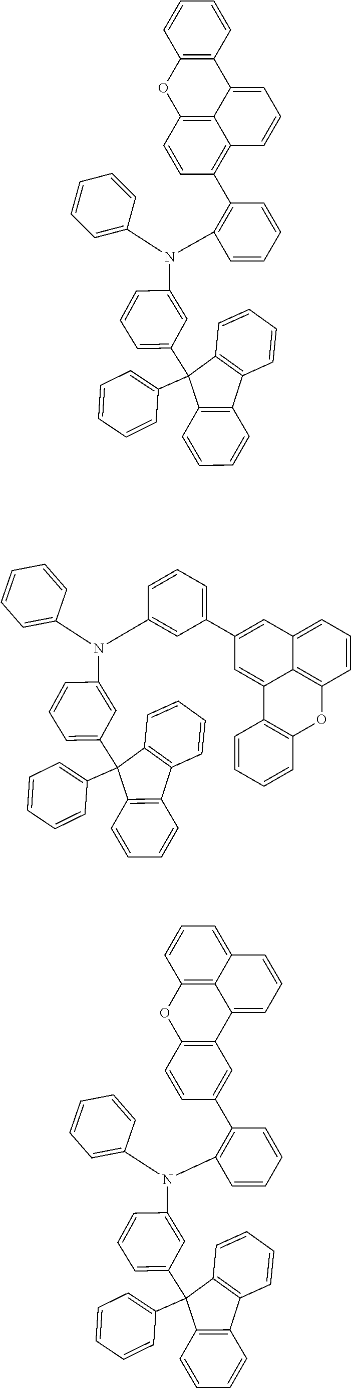 [SMILES: c1ccc(N(c2cccc(C3(c4ccccc4)c4ccccc4-c4ccccc43)c2)c2ccccc2-c2ccc3c4c(cccc24)-c2ccccc2O3)cc1 ; c1ccc(N(c2cccc(-c3cc4c5c(cccc5c3)Oc3ccccc3-4)c2)c2cccc(C3(c4ccccc4)c4ccccc4-c4ccccc43)c2)cc1 ; c1ccc(N(c2cccc(C3(c4ccccc4)c4ccccc4-c4ccccc43)c2)c2ccccc2-c2ccc3c(c2)-c2cccc4cccc(c24)O3)cc1]

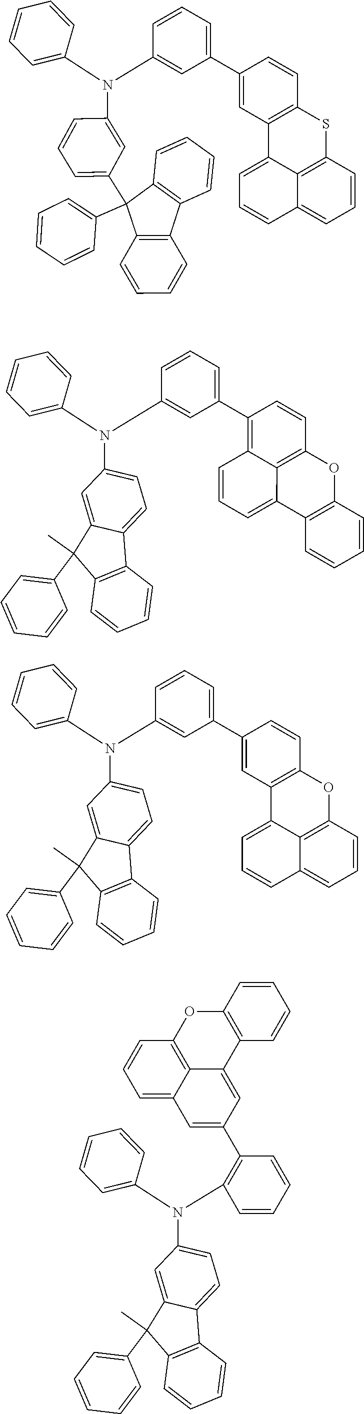

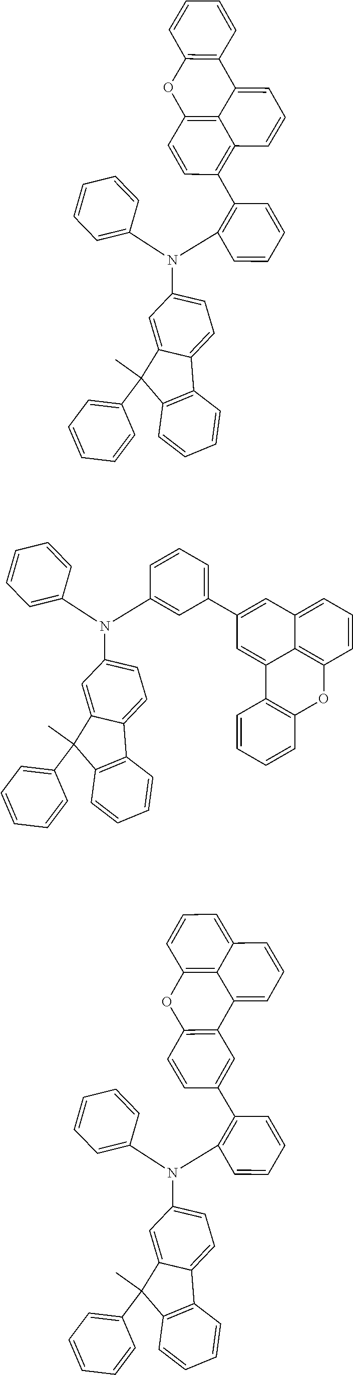 [SMILES: CC1(c2ccccc2)c2ccccc2-c2ccc(N(c3ccccc3)c3ccccc3-c3ccc4c5c(cccc35)-c3ccccc3O4)cc21 ; CC1(c2ccccc2)c2ccccc2-c2ccc(N(c3ccccc3)c3cccc(-c4cc5c6c(cccc6c4)Oc4ccccc4-5)c3)cc21 ; CC1(c2ccccc2)c2ccccc2-c2ccc(N(c3ccccc3)c3ccccc3-c3ccc4c(c3)-c3cccc5cccc(c35)O4)cc21]

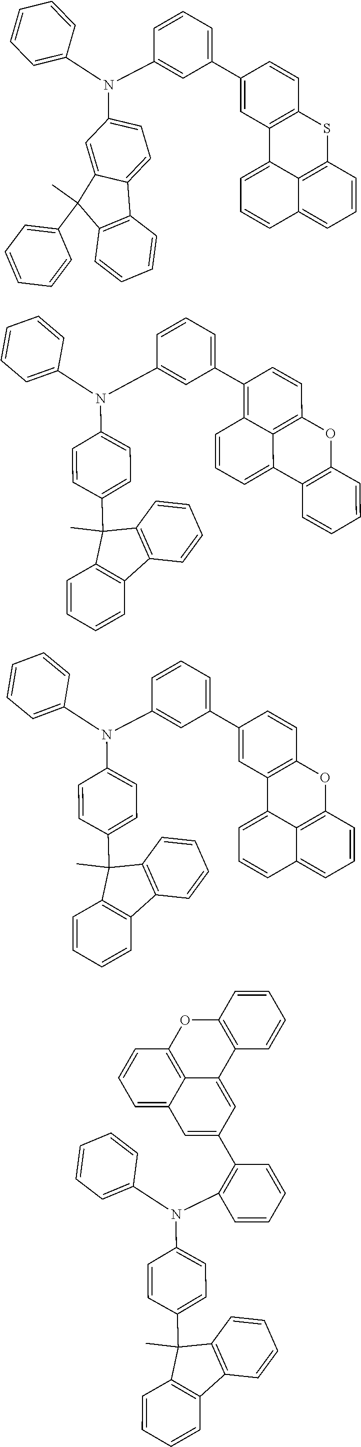

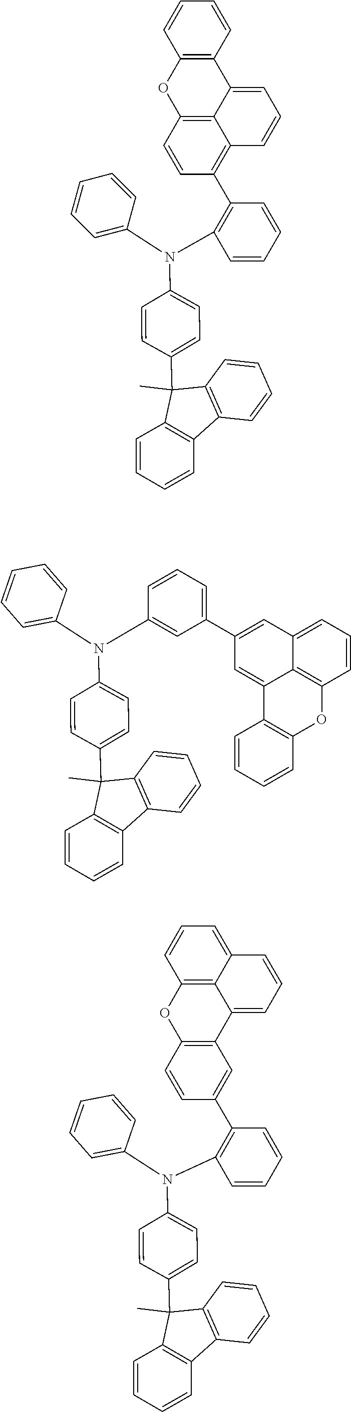 [SMILES: CC1(c2ccc(N(c3ccccc3)c3ccccc3-c3ccc4c5c(cccc35)-c3ccccc3O4)cc2)c2ccccc2-c2ccccc21 ; CC1(c2ccc(N(c3ccccc3)c3cccc(-c4cc5c6c(cccc6c4)Oc4ccccc4-5)c3)cc2)c2ccccc2-c2ccccc21 ; CC1(c2ccc(N(c3ccccc3)c3ccccc3-c3ccc4c(c3)-c3cccc5cccc(c35)O4)cc2)c2ccccc2-c2ccccc21]

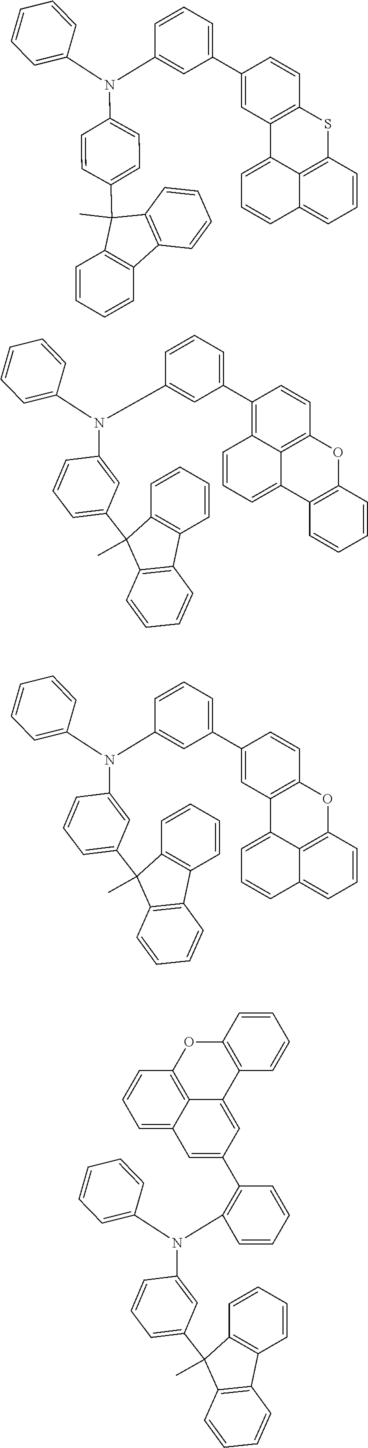 [SMILES: CC1(c2ccc(N(c3ccccc3)c3cccc(-c4ccc5c(c4)-c4cccc6cccc(c46)S5)c3)cc2)c2ccccc2-c2ccccc21 ; CC1(c2cccc(N(c3ccccc3)c3cccc(-c4ccc5c6c(cccc46)-c4ccccc4O5)c3)c2)c2ccccc2-c2ccccc21 ; CC1(c2cccc(N(c3ccccc3)c3cccc(-c4ccc5c(c4)-c4cccc6cccc(c46)O5)c3)c2)c2ccccc2-c2ccccc21 ; CC1(c2cccc(N(c3ccccc3)c3ccccc3-c3cc4c5c(cccc5c3)Oc3ccccc3-4)c2)c2ccccc2-c2ccccc21]

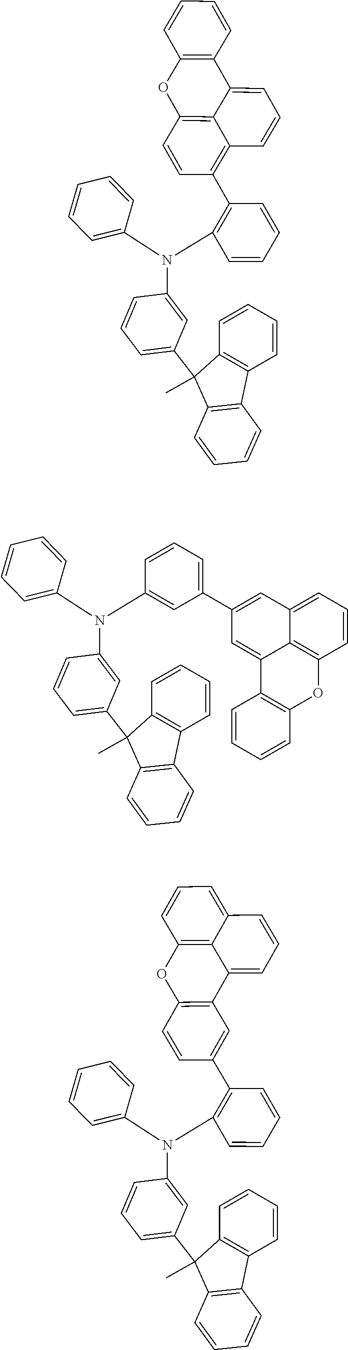 [SMILES: CC1(c2cccc(N(c3ccccc3)c3ccccc3-c3ccc4c5c(cccc35)-c3ccccc3O4)c2)c2ccccc2-c2ccccc21 ; CC1(c2cccc(N(c3ccccc3)c3cccc(-c4cc5c6c(cccc6c4)Oc4ccccc4-5)c3)c2)c2ccccc2-c2ccccc21 ; CC1(c2cccc(N(c3ccccc3)c3ccccc3-c3ccc4c(c3)-c3cccc5cccc(c35)O4)c2)c2ccccc2-c2ccccc21]

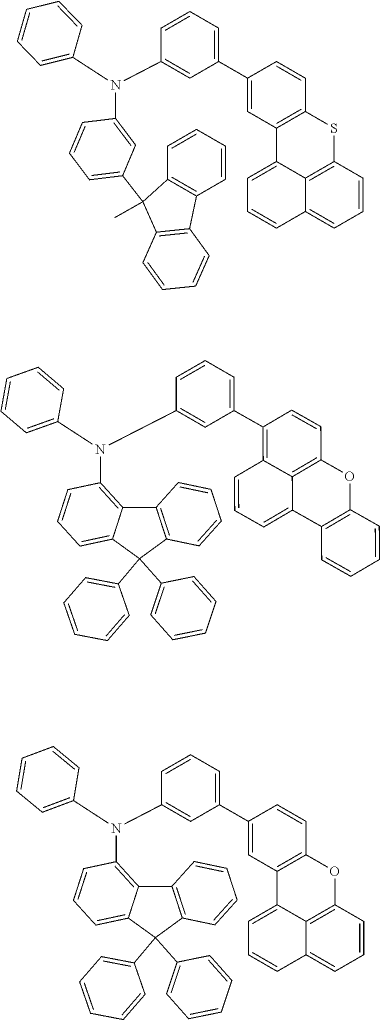 [SMILES: CC1(c2cccc(N(c3ccccc3)c3cccc(-c4ccc5c(c4)-c4cccc6cccc(c46)S5)c3)c2)c2ccccc2-c2ccccc21 ; c1ccc(N(c2cccc(-c3ccc4c5c(cccc35)-c3ccccc3O4)c2)c2cccc3c2-c2ccccc2C3(c2ccccc2)c2ccccc2)cc1 ; c1ccc(N(c2cccc(-c3ccc4c(c3)-c3cccc5cccc(c35)O4)c2)c2cccc3c2-c2ccccc2C3(c2ccccc2)c2ccccc2)cc1]

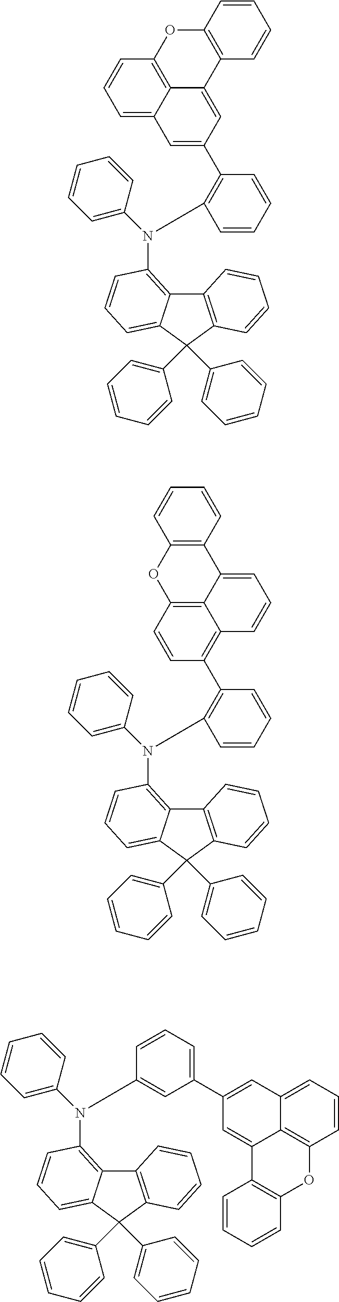 [SMILES: c1ccc(N(c2ccccc2-c2cc3c4c(cccc4c2)Oc2ccccc2-3)c2cccc3c2-c2ccccc2C3(c2ccccc2)c2ccccc2)cc1 ; c1ccc(N(c2ccccc2-c2ccc3c4c(cccc24)-c2ccccc2O3)c2cccc3c2-c2ccccc2C3(c2ccccc2)c2ccccc2)cc1 ; c1ccc(N(c2cccc(-c3cc4c5c(cccc5c3)Oc3ccccc3-4)c2)c2cccc3c2-c2ccccc2C3(c2ccccc2)c2ccccc2)cc1]

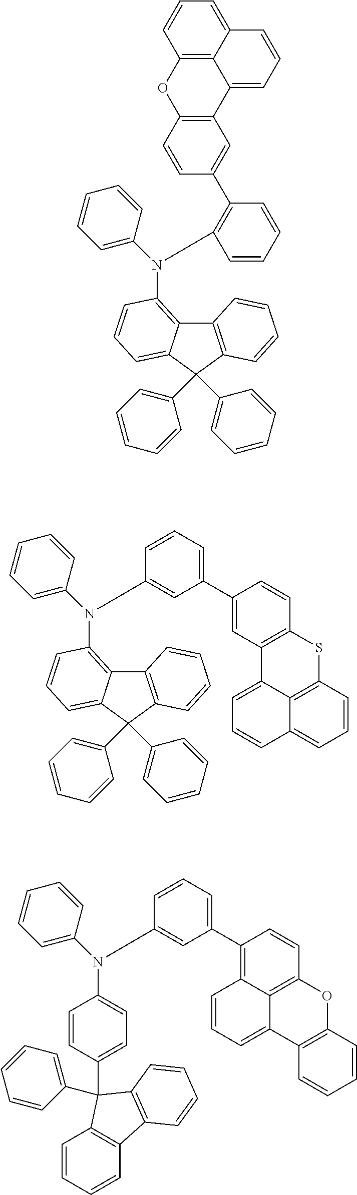 [SMILES: c1ccc(N(c2ccccc2-c2ccc3c(c2)-c2cccc4cccc(c24)O3)c2cccc3c2-c2ccccc2C3(c2ccccc2)c2ccccc2)cc1 ; c1ccc(N(c2cccc(-c3ccc4c(c3)-c3cccc5cccc(c35)S4)c2)c2cccc3c2-c2ccccc2C3(c2ccccc2)c2ccccc2)cc1 ; c1ccc(N(c2ccc(C3(c4ccccc4)c4ccccc4-c4ccccc43)cc2)c2cccc(-c3ccc4c5c(cccc35)-c3ccccc3O4)c2)cc1]

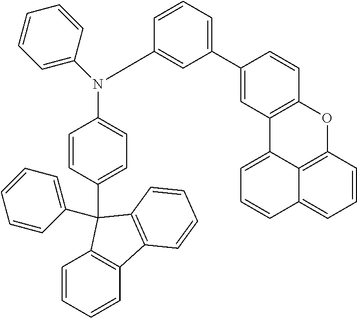 [SMILES: c1ccc(N(c2ccc(C3(c4ccccc4)c4ccccc4-c4ccccc43)cc2)c2cccc(-c3ccc4c(c3)-c3cccc5cccc(c35)O4)c2)cc1]

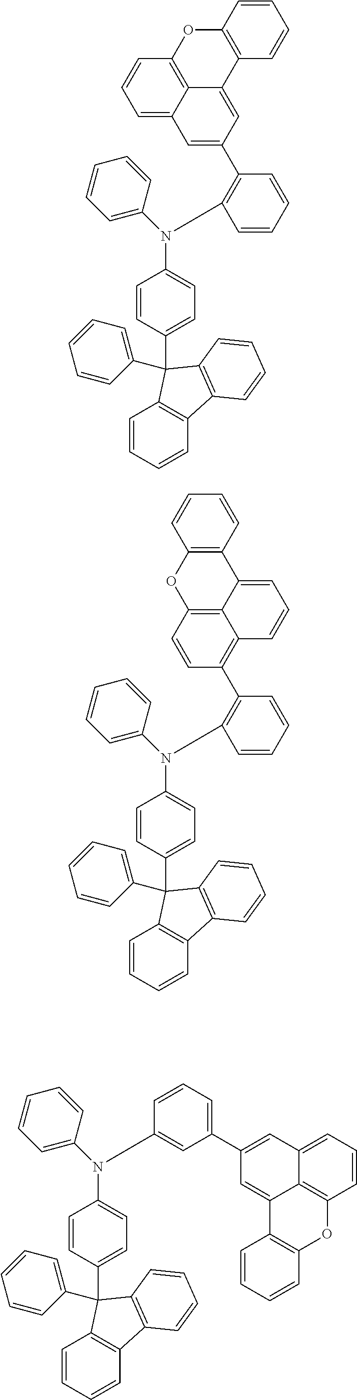

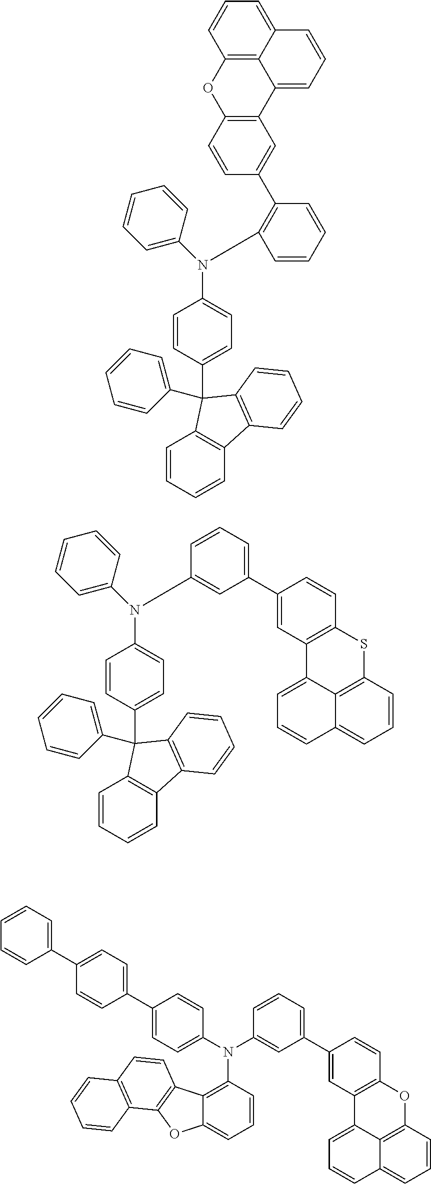

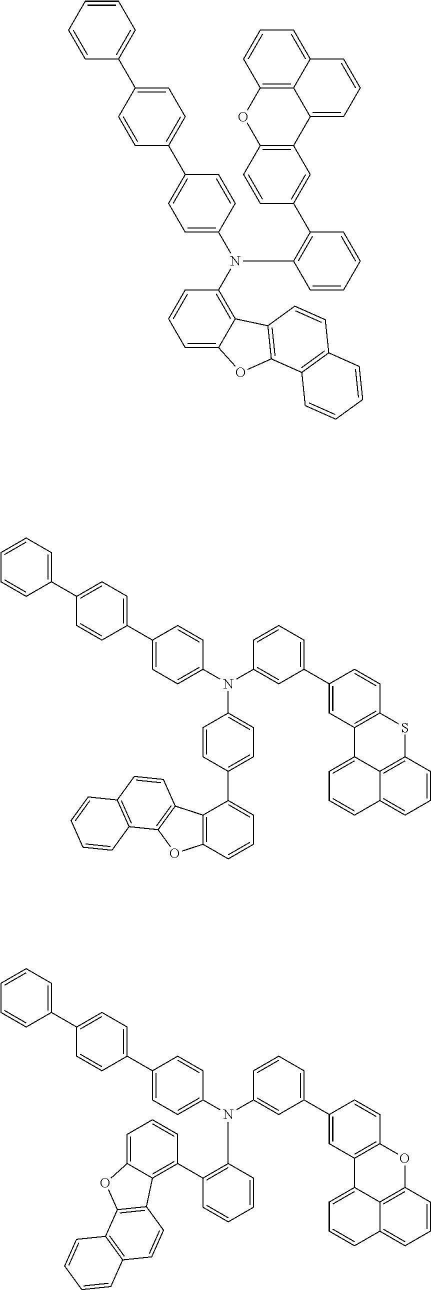 [SMILES: c1ccc(-c2ccc(-c3ccc(N(c4ccccc4-c4ccc5c(c4)-c4cccc6cccc(c46)O5)c4cccc5oc6c7ccccc7ccc6c45)cc3)cc2)cc1 ; c1ccc(-c2ccc(-c3ccc(N(c4ccc(-c5cccc6oc7c8ccccc8ccc7c56)cc4)c4cccc(-c5ccc6c(c5)-c5cccc7cccc(c57)S6)c4)cc3)cc2)cc1 ; c1ccc(-c2ccc(-c3ccc(N(c4cccc(-c5ccc6c(c5)-c5cccc7cccc(c57)O6)c4)c4ccccc4-c4cccc5oc6c7ccccc7ccc6c45)cc3)cc2)cc1]

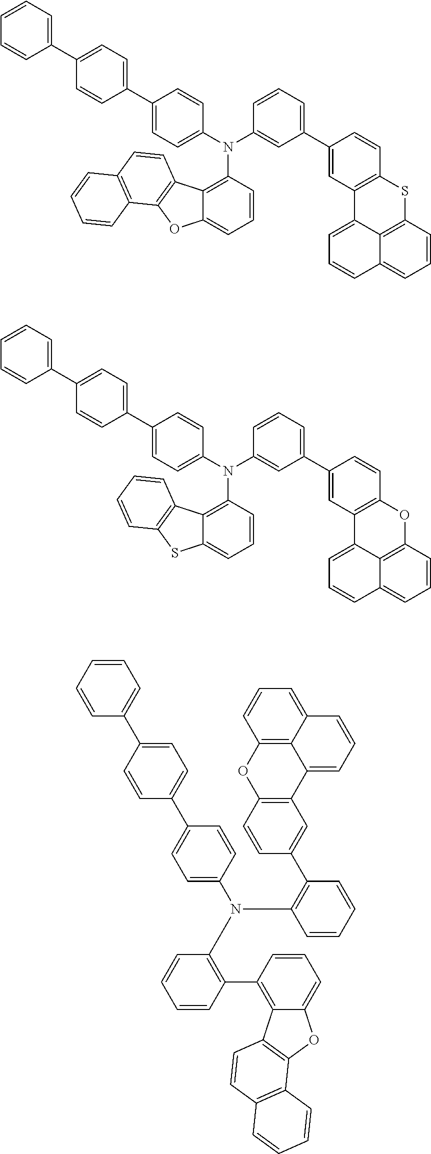 [SMILES: c1ccc(-c2ccc(-c3ccc(N(c4cccc(-c5ccc6c(c5)-c5cccc7cccc(c57)S6)c4)c4cccc5oc6c7ccccc7ccc6c45)cc3)cc2)cc1 ; c1ccc(-c2ccc(-c3ccc(N(c4cccc(-c5ccc6c(c5)-c5cccc7cccc(c57)O6)c4)c4cccc5sc6ccccc6c45)cc3)cc2)cc1 ; c1ccc(-c2ccc(-c3ccc(N(c4ccccc4-c4ccc5c(c4)-c4cccc6cccc(c46)O5)c4ccccc4-c4cccc5oc6c7ccccc7ccc6c45)cc3)cc2)cc1]

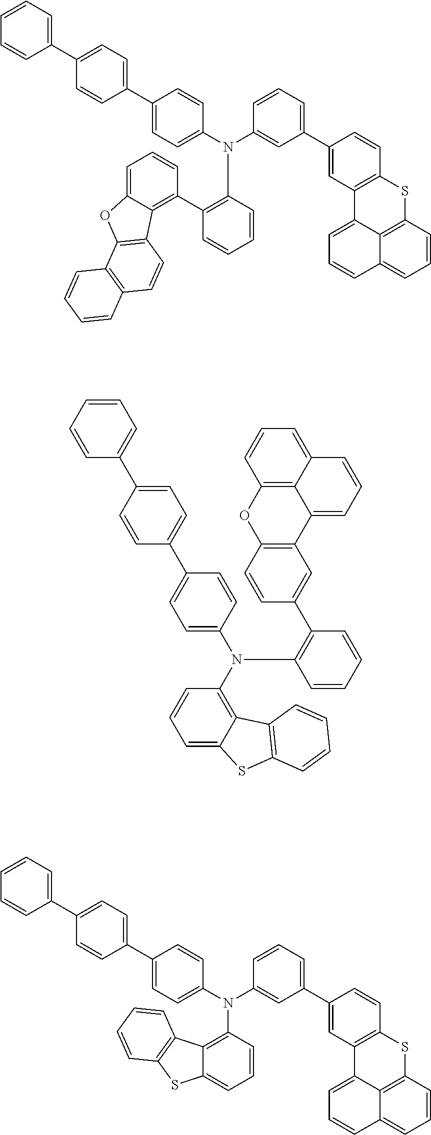 [SMILES: c1ccc(-c2ccc(-c3ccc(N(c4cccc(-c5ccc6c(c5)-c5cccc7cccc(c57)S6)c4)c4ccccc4-c4cccc5oc6c7ccccc7ccc6c45)cc3)cc2)cc1 ; c1ccc(-c2ccc(-c3ccc(N(c4ccccc4-c4ccc5c(c4)-c4cccc6cccc(c46)O5)c4cccc5sc6ccccc6c45)cc3)cc2)cc1 ; c1ccc(-c2ccc(-c3ccc(N(c4cccc(-c5ccc6c(c5)-c5cccc7cccc(c57)S6)c4)c4cccc5sc6ccccc6c45)cc3)cc2)cc1]

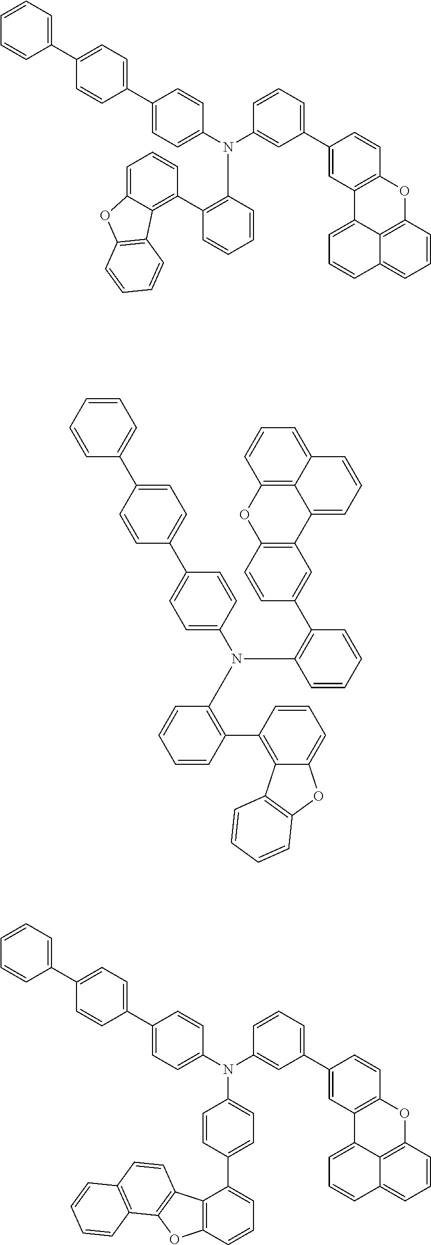 [SMILES: c1ccc(-c2ccc(-c3ccc(N(c4cccc(-c5ccc6c(c5)-c5cccc7cccc(c57)O6)c4)c4ccccc4-c4cccc5oc6ccccc6c45)cc3)cc2)cc1 ; c1ccc(-c2ccc(-c3ccc(N(c4ccccc4-c4ccc5c(c4)-c4cccc6cccc(c46)O5)c4ccccc4-c4cccc5oc6ccccc6c45)cc3)cc2)cc1 ; c1ccc(-c2ccc(-c3ccc(N(c4ccc(-c5cccc6oc7c8ccccc8ccc7c56)cc4)c4cccc(-c5ccc6c(c5)-c5cccc7cccc(c57)O6)c4)cc3)cc2)cc1]

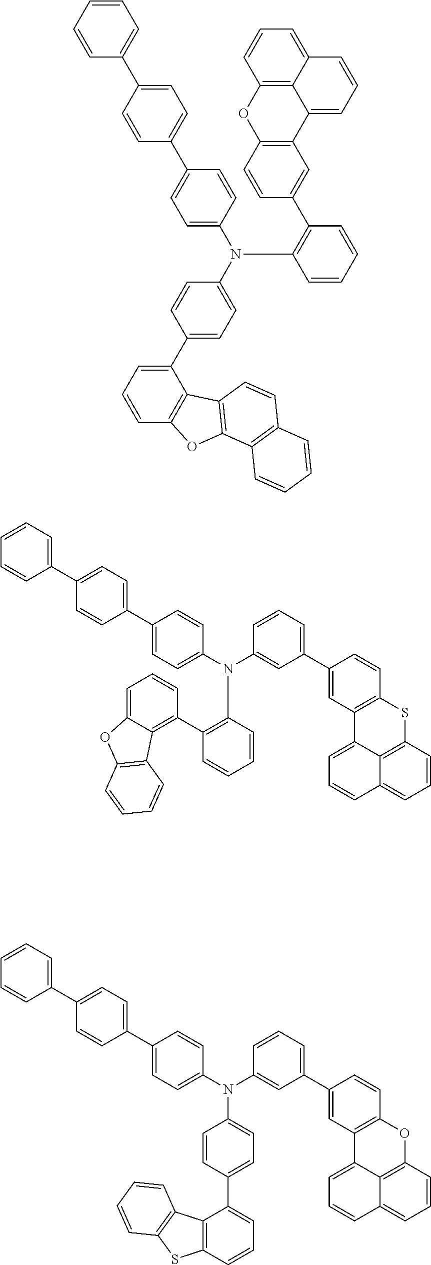 [SMILES: c1ccc(-c2ccc(-c3ccc(N(c4ccc(-c5cccc6oc7c8ccccc8ccc7c56)cc4)c4ccccc4-c4ccc5c(c4)-c4cccc6cccc(c46)O5)cc3)cc2)cc1 ; c1ccc(-c2ccc(-c3ccc(N(c4cccc(-c5ccc6c(c5)-c5cccc7cccc(c57)S6)c4)c4ccccc4-c4cccc5oc6ccccc6c45)cc3)cc2)cc1 ; c1ccc(-c2ccc(-c3ccc(N(c4ccc(-c5cccc6sc7ccccc7c56)cc4)c4cccc(-c5ccc6c(c5)-c5cccc7cccc(c57)O6)c4)cc3)cc2)cc1]

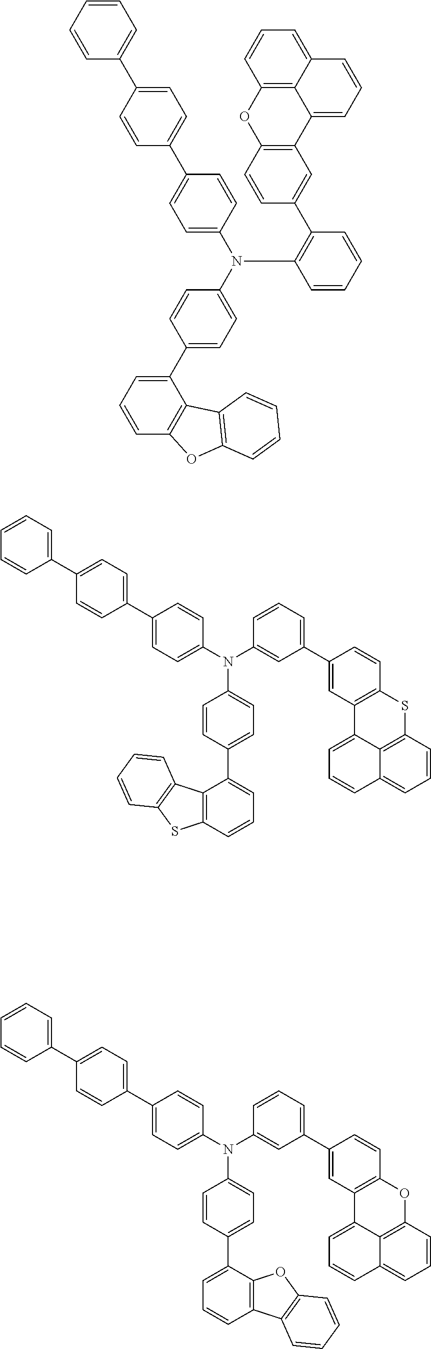 [SMILES: c1ccc(-c2ccc(-c3ccc(N(c4ccc(-c5cccc6oc7ccccc7c56)cc4)c4ccccc4-c4ccc5c(c4)-c4cccc6cccc(c46)O5)cc3)cc2)cc1 ; c1ccc(-c2ccc(-c3ccc(N(c4ccc(-c5cccc6sc7ccccc7c56)cc4)c4cccc(-c5ccc6c(c5)-c5cccc7cccc(c57)S6)c4)cc3)cc2)cc1 ; c1ccc(-c2ccc(-c3ccc(N(c4ccc(-c5cccc6c5oc5ccccc56)cc4)c4cccc(-c5ccc6c(c5)-c5cccc7cccc(c57)O6)c4)cc3)cc2)cc1]

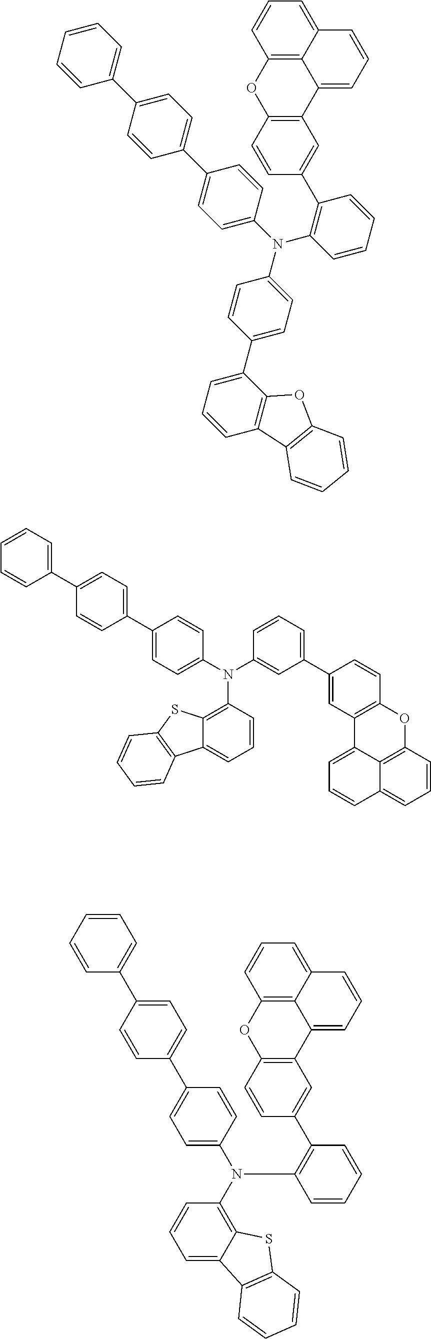 [SMILES: c1ccc(-c2ccc(-c3ccc(N(c4ccc(-c5cccc6c5oc5ccccc56)cc4)c4ccccc4-c4ccc5c(c4)-c4cccc6cccc(c46)O5)cc3)cc2)cc1 ; c1ccc(-c2ccc(-c3ccc(N(c4cccc(-c5ccc6c(c5)-c5cccc7cccc(c57)O6)c4)c4cccc5c4sc4ccccc45)cc3)cc2)cc1 ; c1ccc(-c2ccc(-c3ccc(N(c4ccccc4-c4ccc5c(c4)-c4cccc6cccc(c46)O5)c4cccc5c4sc4ccccc45)cc3)cc2)cc1]

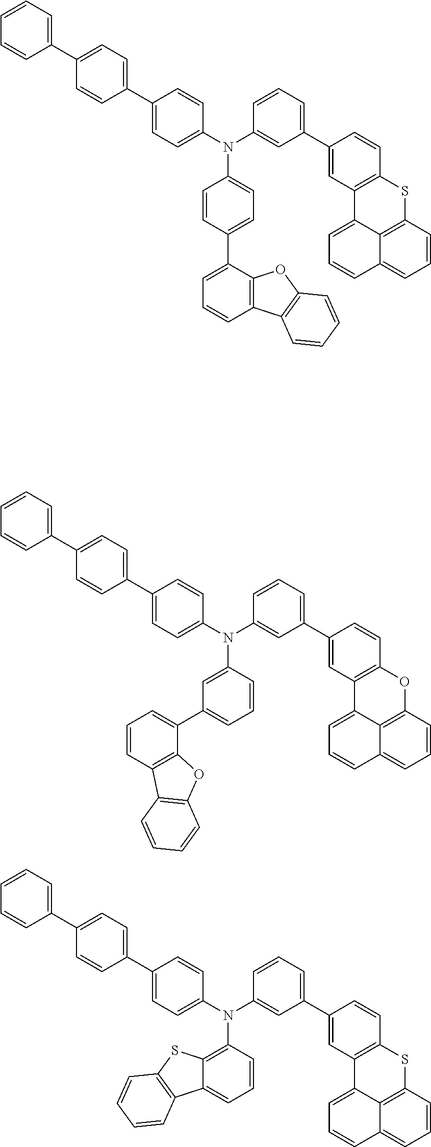 [SMILES: c1ccc(-c2ccc(-c3ccc(N(c4ccc(-c5cccc6c5oc5ccccc56)cc4)c4cccc(-c5ccc6c(c5)-c5cccc7cccc(c57)S6)c4)cc3)cc2)cc1 ; c1ccc(-c2ccc(-c3ccc(N(c4cccc(-c5ccc6c(c5)-c5cccc7cccc(c57)O6)c4)c4cccc(-c5cccc6c5oc5ccccc56)c4)cc3)cc2)cc1 ; c1ccc(-c2ccc(-c3ccc(N(c4cccc(-c5ccc6c(c5)-c5cccc7cccc(c57)S6)c4)c4cccc5c4sc4ccccc45)cc3)cc2)cc1]

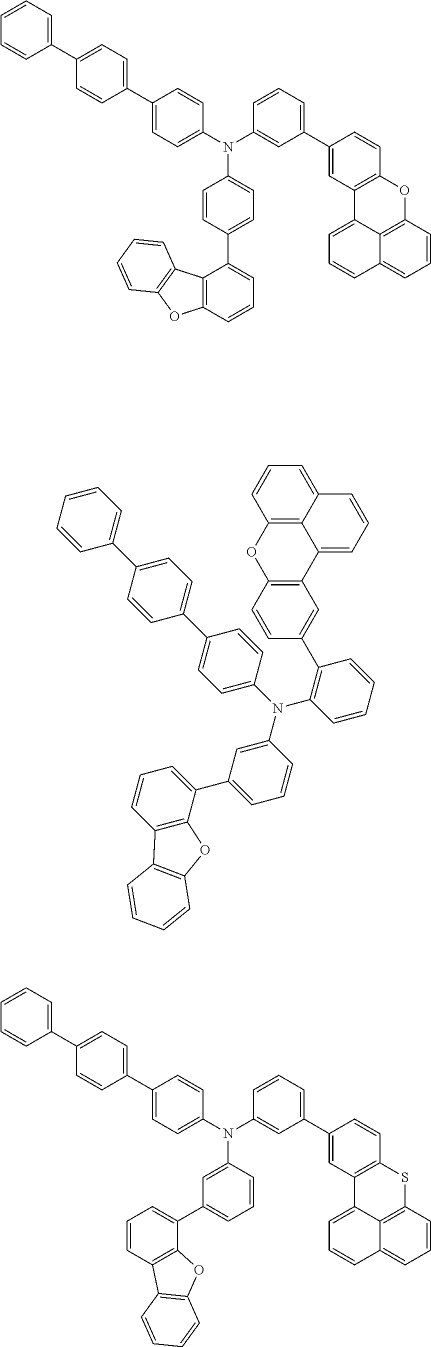 [SMILES: c1ccc(-c2ccc(-c3ccc(N(c4ccc(-c5cccc6oc7ccccc7c56)cc4)c4cccc(-c5ccc6c(c5)-c5cccc7cccc(c57)O6)c4)cc3)cc2)cc1 ; c1ccc(-c2ccc(-c3ccc(N(c4cccc(-c5cccc6c5oc5ccccc56)c4)c4ccccc4-c4ccc5c(c4)-c4cccc6cccc(c46)O5)cc3)cc2)cc1 ; c1ccc(-c2ccc(-c3ccc(N(c4cccc(-c5ccc6c(c5)-c5cccc7cccc(c57)S6)c4)c4cccc(-c5cccc6c5oc5ccccc56)c4)cc3)cc2)cc1]

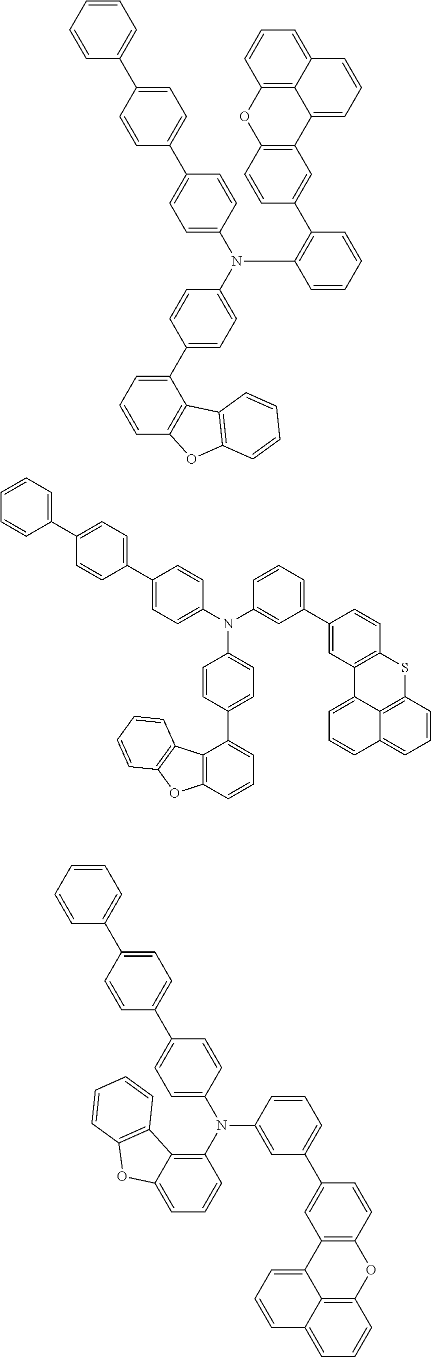 [SMILES: c1ccc(-c2ccc(-c3ccc(N(c4ccc(-c5cccc6oc7ccccc7c56)cc4)c4ccccc4-c4ccc5c(c4)-c4cccc6cccc(c46)O5)cc3)cc2)cc1 ; c1ccc(-c2ccc(-c3ccc(N(c4ccc(-c5cccc6oc7ccccc7c56)cc4)c4cccc(-c5ccc6c(c5)-c5cccc7cccc(c57)S6)c4)cc3)cc2)cc1 ; c1ccc(-c2ccc(-c3ccc(N(c4cccc(-c5ccc6c(c5)-c5cccc7cccc(c57)O6)c4)c4cccc5oc6ccccc6c45)cc3)cc2)cc1]

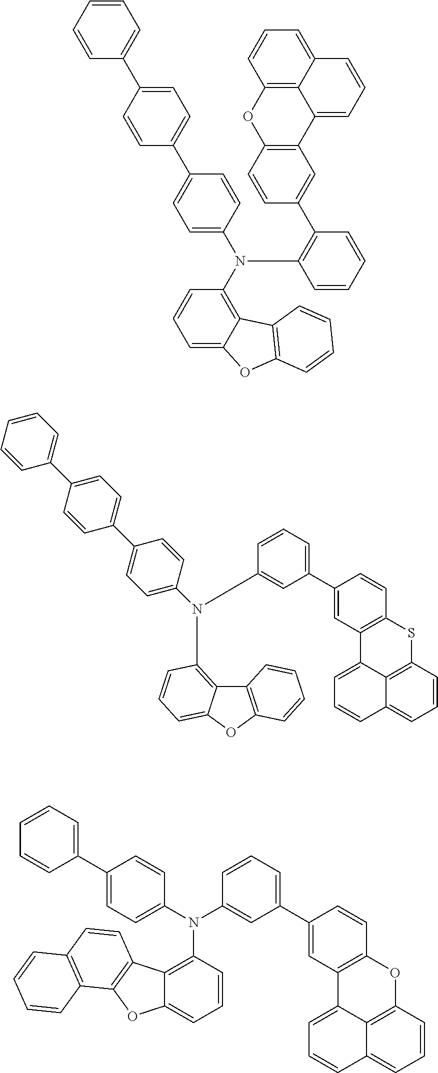 [SMILES: c1ccc(-c2ccc(-c3ccc(N(c4ccccc4-c4ccc5c(c4)-c4cccc6cccc(c46)O5)c4cccc5oc6ccccc6c45)cc3)cc2)cc1 ; c1ccc(-c2ccc(-c3ccc(N(c4cccc(-c5ccc6c(c5)-c5cccc7cccc(c57)S6)c4)c4cccc5oc6ccccc6c45)cc3)cc2)cc1 ; c1ccc(-c2ccc(N(c3cccc(-c4ccc5c(c4)-c4cccc6cccc(c46)O5)c3)c3cccc4oc5c6ccccc6ccc5c34)cc2)cc1]

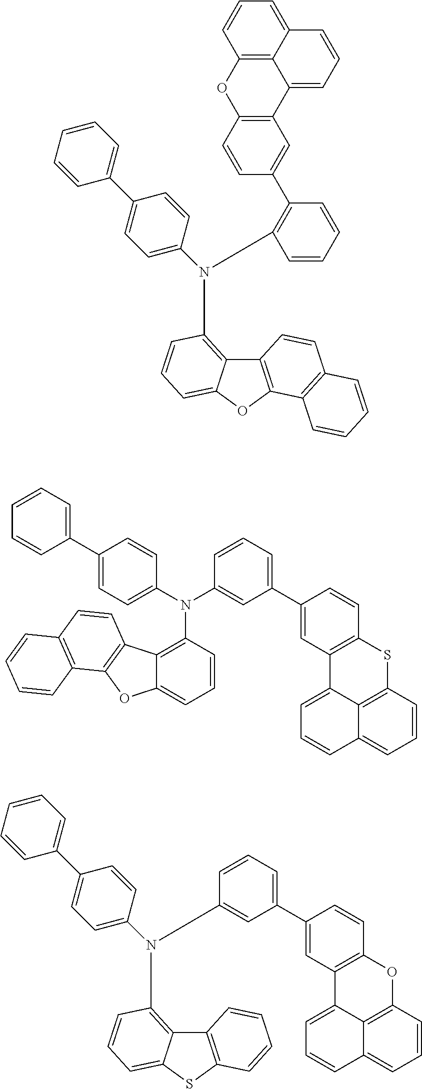 [SMILES: c1ccc(-c2ccc(N(c3ccccc3-c3ccc4c(c3)-c3cccc5cccc(c35)O4)c3cccc4oc5c6ccccc6ccc5c34)cc2)cc1 ; c1ccc(-c2ccc(N(c3cccc(-c4ccc5c(c4)-c4cccc6cccc(c46)S5)c3)c3cccc4oc5c6ccccc6ccc5c34)cc2)cc1 ; c1ccc(-c2ccc(N(c3cccc(-c4ccc5c(c4)-c4cccc6cccc(c46)O5)c3)c3cccc4sc5ccccc5c34)cc2)cc1]

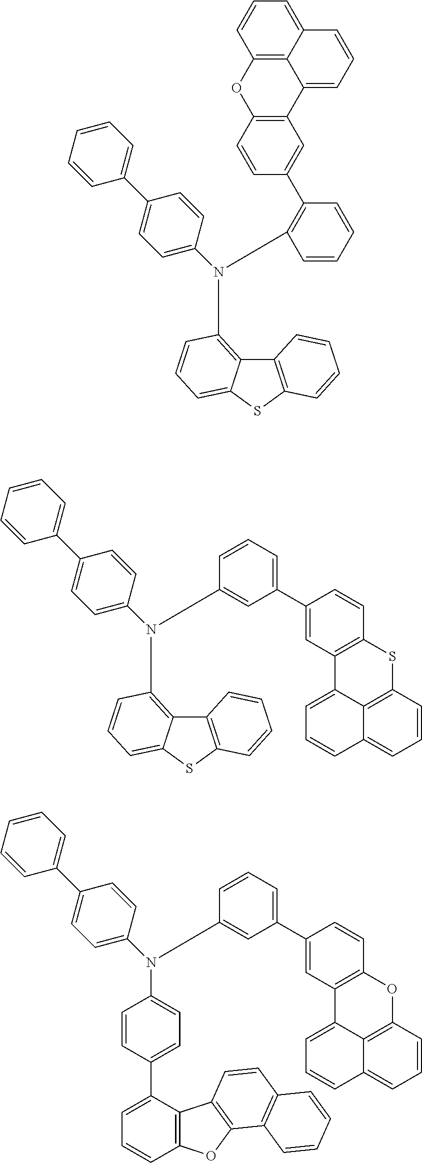 [SMILES: c1ccc(-c2ccc(N(c3ccccc3-c3ccc4c(c3)-c3cccc5cccc(c35)O4)c3cccc4sc5ccccc5c34)cc2)cc1 ; c1ccc(-c2ccc(N(c3cccc(-c4ccc5c(c4)-c4cccc6cccc(c46)S5)c3)c3cccc4sc5ccccc5c34)cc2)cc1 ; c1ccc(-c2ccc(N(c3ccc(-c4cccc5oc6c7ccccc7ccc6c45)cc3)c3cccc(-c4ccc5c(c4)-c4cccc6cccc(c46)O5)c3)cc2)cc1]

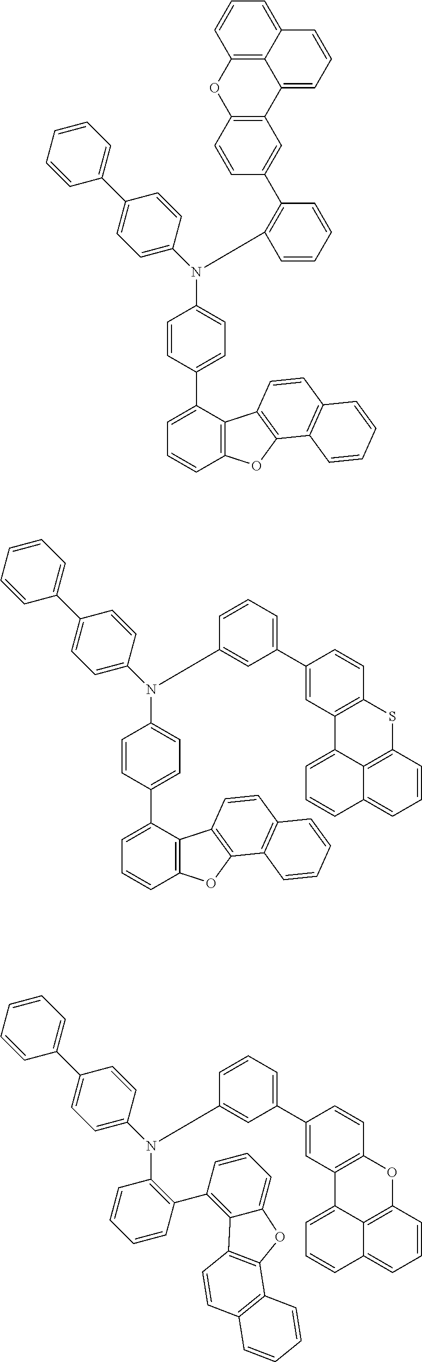 [SMILES: c1ccc(-c2ccc(N(c3ccc(-c4cccc5oc6c7ccccc7ccc6c45)cc3)c3ccccc3-c3ccc4c(c3)-c3cccc5cccc(c35)O4)cc2)cc1 ; c1ccc(-c2ccc(N(c3ccc(-c4cccc5oc6c7ccccc7ccc6c45)cc3)c3cccc(-c4ccc5c(c4)-c4cccc6cccc(c46)S5)c3)cc2)cc1 ; c1ccc(-c2ccc(N(c3cccc(-c4ccc5c(c4)-c4cccc6cccc(c46)O5)c3)c3ccccc3-c3cccc4oc5c6ccccc6ccc5c34)cc2)cc1]

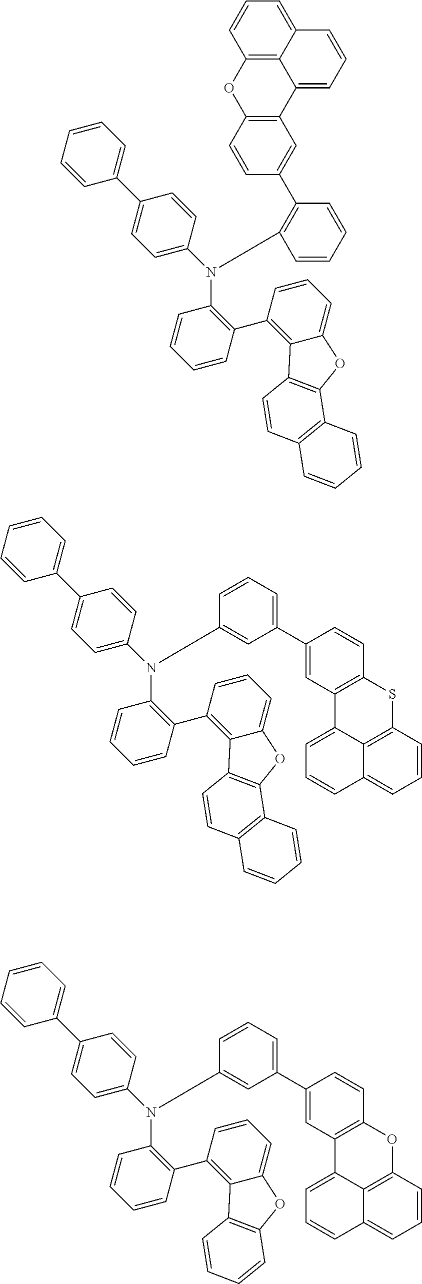 [SMILES: c1ccc(-c2ccc(N(c3ccccc3-c3ccc4c(c3)-c3cccc5cccc(c35)O4)c3ccccc3-c3cccc4oc5c6ccccc6ccc5c34)cc2)cc1 ; c1ccc(-c2ccc(N(c3cccc(-c4ccc5c(c4)-c4cccc6cccc(c46)S5)c3)c3ccccc3-c3cccc4oc5c6ccccc6ccc5c34)cc2)cc1 ; c1ccc(-c2ccc(N(c3cccc(-c4ccc5c(c4)-c4cccc6cccc(c46)O5)c3)c3ccccc3-c3cccc4oc5ccccc5c34)cc2)cc1]

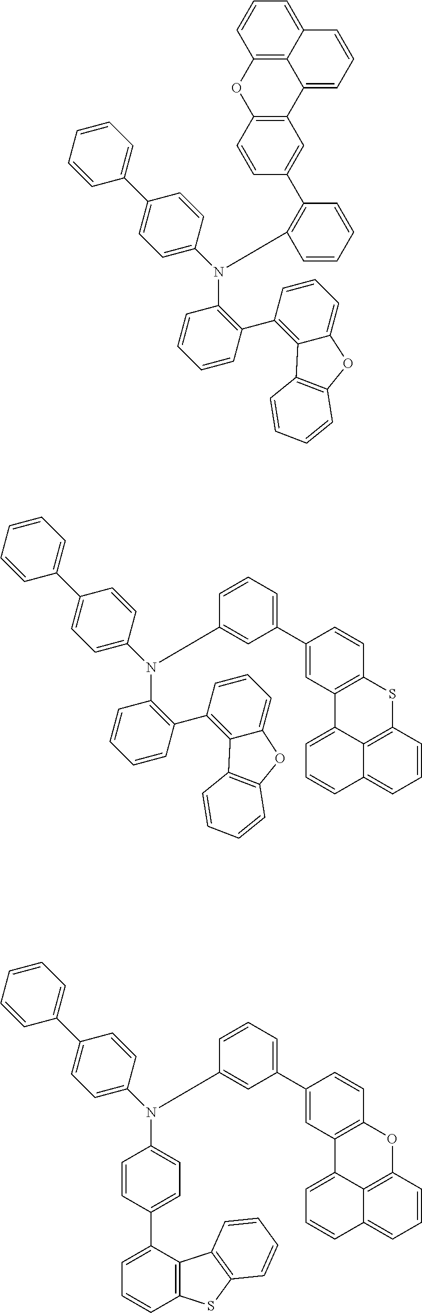 [SMILES: c1ccc(-c2ccc(N(c3ccccc3-c3ccc4c(c3)-c3cccc5cccc(c35)O4)c3ccccc3-c3cccc4oc5ccccc5c34)cc2)cc1 ; c1ccc(-c2ccc(N(c3cccc(-c4ccc5c(c4)-c4cccc6cccc(c46)S5)c3)c3ccccc3-c3cccc4oc5ccccc5c34)cc2)cc1 ; c1ccc(-c2ccc(N(c3ccc(-c4cccc5sc6ccccc6c45)cc3)c3cccc(-c4ccc5c(c4)-c4cccc6cccc(c46)O5)c3)cc2)cc1]

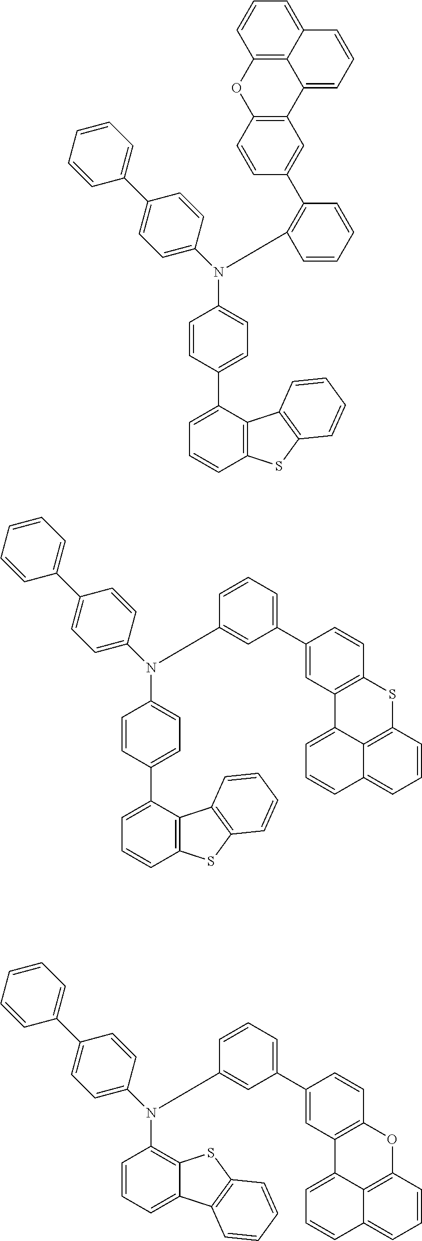 [SMILES: c1ccc(-c2ccc(N(c3ccc(-c4cccc5sc6ccccc6c45)cc3)c3ccccc3-c3ccc4c(c3)-c3cccc5cccc(c35)O4)cc2)cc1 ; c1ccc(-c2ccc(N(c3ccc(-c4cccc5sc6ccccc6c45)cc3)c3cccc(-c4ccc5c(c4)-c4cccc6cccc(c46)S5)c3)cc2)cc1 ; c1ccc(-c2ccc(N(c3cccc(-c4ccc5c(c4)-c4cccc6cccc(c46)O5)c3)c3cccc4c3sc3ccccc34)cc2)cc1]

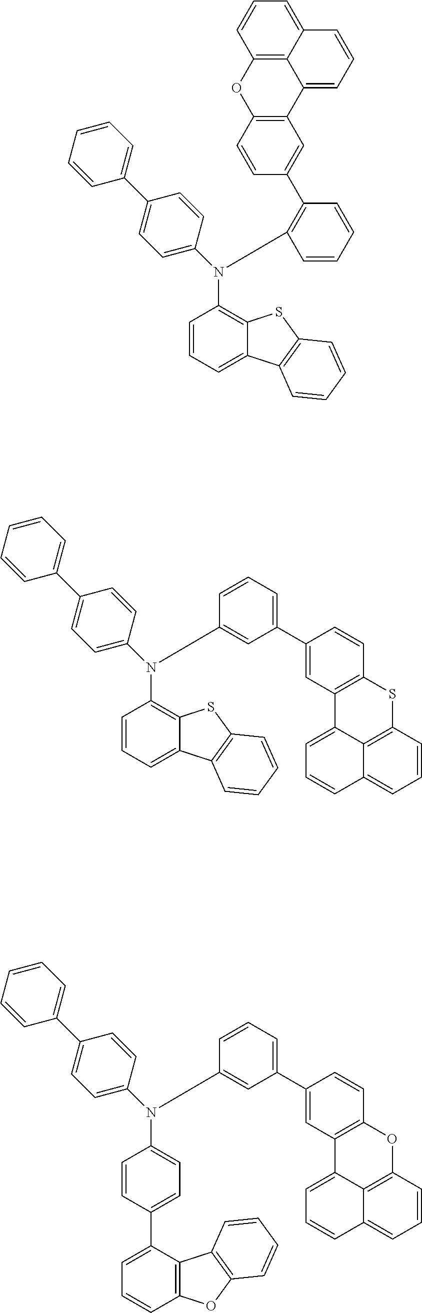 [SMILES: c1ccc(-c2ccc(N(c3ccccc3-c3ccc4c(c3)-c3cccc5cccc(c35)O4)c3cccc4c3sc3ccccc34)cc2)cc1 ; c1ccc(-c2ccc(N(c3cccc(-c4ccc5c(c4)-c4cccc6cccc(c46)S5)c3)c3cccc4c3sc3ccccc34)cc2)cc1 ; c1ccc(-c2ccc(N(c3ccc(-c4cccc5oc6ccccc6c45)cc3)c3cccc(-c4ccc5c(c4)-c4cccc6cccc(c46)O5)c3)cc2)cc1]

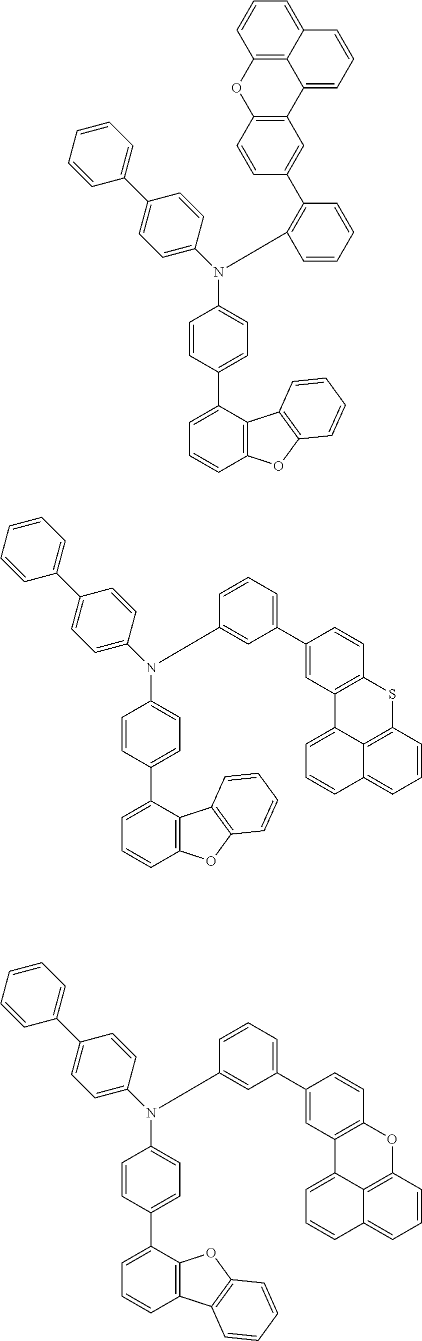 [SMILES: c1ccc(-c2ccc(N(c3ccc(-c4cccc5oc6ccccc6c45)cc3)c3ccccc3-c3ccc4c(c3)-c3cccc5cccc(c35)O4)cc2)cc1 ; c1ccc(-c2ccc(N(c3ccc(-c4cccc5oc6ccccc6c45)cc3)c3cccc(-c4ccc5c(c4)-c4cccc6cccc(c46)S5)c3)cc2)cc1 ; c1ccc(-c2ccc(N(c3ccc(-c4cccc5c4oc4ccccc45)cc3)c3cccc(-c4ccc5c(c4)-c4cccc6cccc(c46)O5)c3)cc2)cc1]

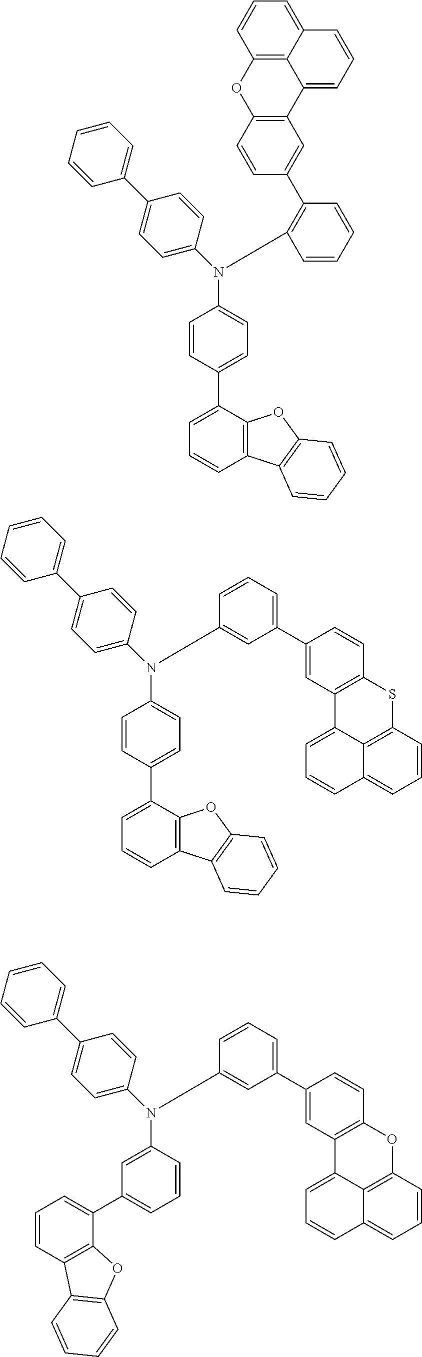 [SMILES: c1ccc(-c2ccc(N(c3ccc(-c4cccc5c4oc4ccccc45)cc3)c3ccccc3-c3ccc4c(c3)-c3cccc5cccc(c35)O4)cc2)cc1 ; c1ccc(-c2ccc(N(c3ccc(-c4cccc5c4oc4ccccc45)cc3)c3cccc(-c4ccc5c(c4)-c4cccc6cccc(c46)S5)c3)cc2)cc1 ; c1ccc(-c2ccc(N(c3cccc(-c4ccc5c(c4)-c4cccc6cccc(c46)O5)c3)c3cccc(-c4cccc5c4oc4ccccc45)c3)cc2)cc1]

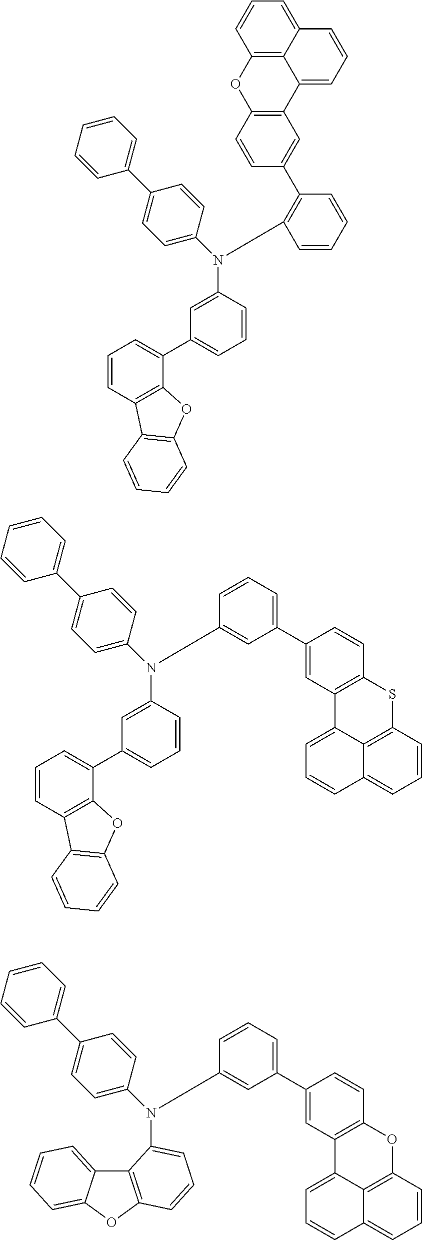 [SMILES: c1ccc(-c2ccc(N(c3cccc(-c4cccc5c4oc4ccccc45)c3)c3ccccc3-c3ccc4c(c3)-c3cccc5cccc(c35)O4)cc2)cc1 ; c1ccc(-c2ccc(N(c3cccc(-c4ccc5c(c4)-c4cccc6cccc(c46)S5)c3)c3cccc(-c4cccc5c4oc4ccccc45)c3)cc2)cc1 ; c1ccc(-c2ccc(N(c3cccc(-c4ccc5c(c4)-c4cccc6cccc(c46)O5)c3)c3cccc4oc5ccccc5c34)cc2)cc1]

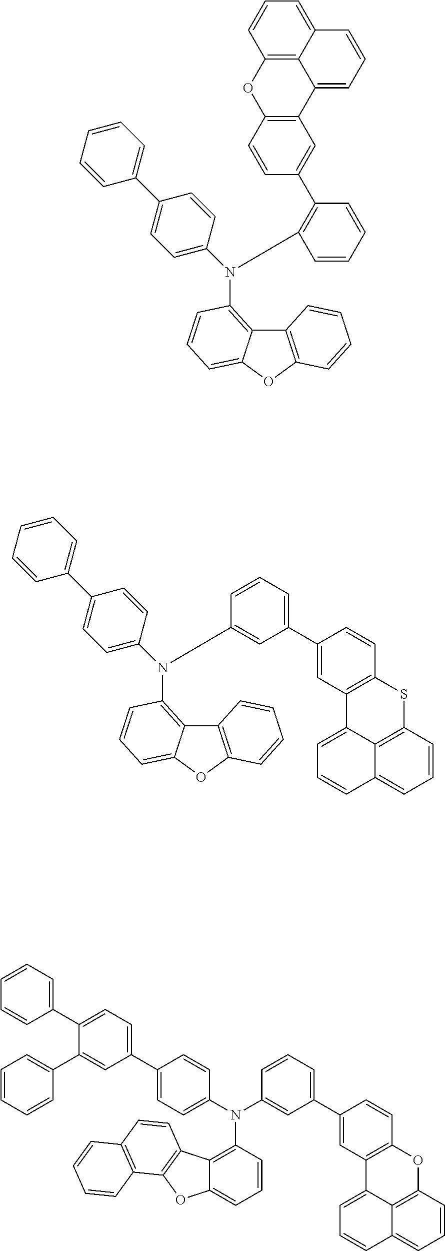 [SMILES: c1ccc(-c2ccc(N(c3ccccc3-c3ccc4c(c3)-c3cccc5cccc(c35)O4)c3cccc4oc5ccccc5c34)cc2)cc1 ; c1ccc(-c2ccc(N(c3cccc(-c4ccc5c(c4)-c4cccc6cccc(c46)S5)c3)c3cccc4oc5ccccc5c34)cc2)cc1 ; c1ccc(-c2ccc(-c3ccc(N(c4cccc(-c5ccc6c(c5)-c5cccc7cccc(c57)O6)c4)c4cccc5oc6c7ccccc7ccc6c45)cc3)cc2-c2ccccc2)cc1]

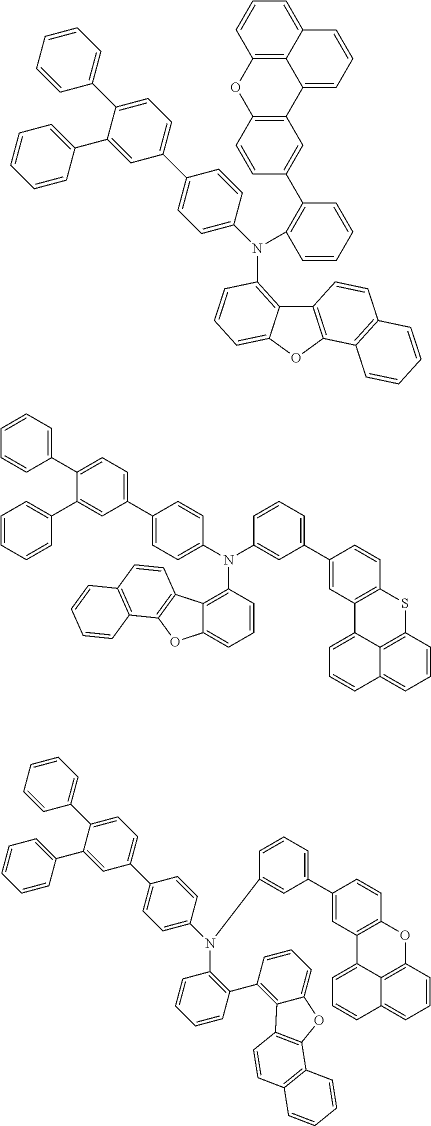 [SMILES: c1ccc(-c2ccc(-c3ccc(N(c4ccccc4-c4ccc5c(c4)-c4cccc6cccc(c46)O5)c4cccc5oc6c7ccccc7ccc6c45)cc3)cc2-c2ccccc2)cc1 ; c1ccc(-c2ccc(-c3ccc(N(c4cccc(-c5ccc6c(c5)-c5cccc7cccc(c57)S6)c4)c4cccc5oc6c7ccccc7ccc6c45)cc3)cc2-c2ccccc2)cc1 ; c1ccc(-c2ccc(-c3ccc(N(c4cccc(-c5ccc6c(c5)-c5cccc7cccc(c57)O6)c4)c4ccccc4-c4cccc5oc6c7ccccc7ccc6c45)cc3)cc2-c2ccccc2)cc1]

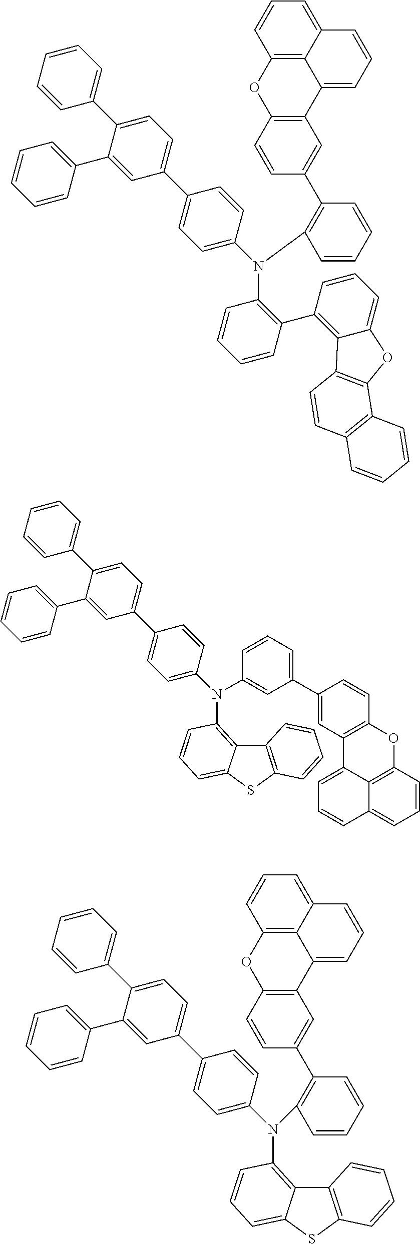 [SMILES: c1ccc(-c2ccc(-c3ccc(N(c4ccccc4-c4ccc5c(c4)-c4cccc6cccc(c46)O5)c4ccccc4-c4cccc5oc6c7ccccc7ccc6c45)cc3)cc2-c2ccccc2)cc1 ; c1ccc(-c2ccc(-c3ccc(N(c4cccc(-c5ccc6c(c5)-c5cccc7cccc(c57)O6)c4)c4cccc5sc6ccccc6c45)cc3)cc2-c2ccccc2)cc1 ; c1ccc(-c2ccc(-c3ccc(N(c4ccccc4-c4ccc5c(c4)-c4cccc6cccc(c46)O5)c4cccc5sc6ccccc6c45)cc3)cc2-c2ccccc2)cc1]

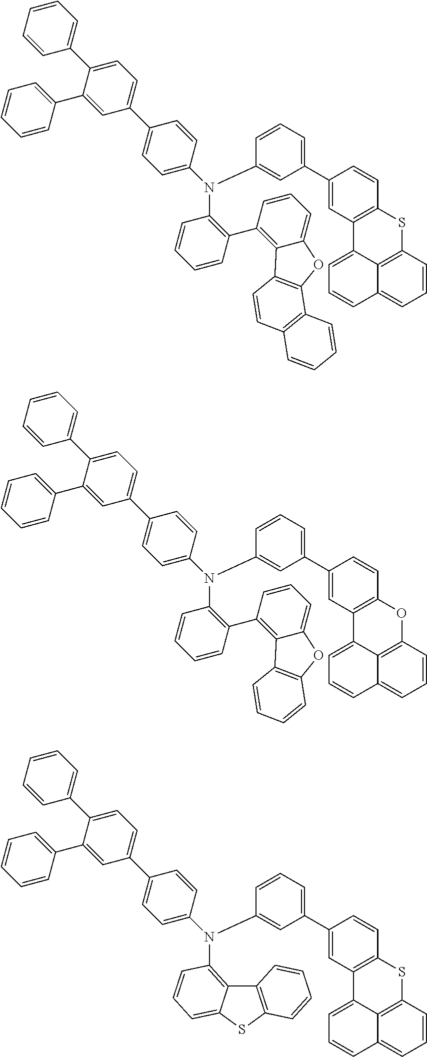 [SMILES: c1ccc(-c2ccc(-c3ccc(N(c4cccc(-c5ccc6c(c5)-c5cccc7cccc(c57)S6)c4)c4ccccc4-c4cccc5oc6c7ccccc7ccc6c45)cc3)cc2-c2ccccc2)cc1 ; c1ccc(-c2ccc(-c3ccc(N(c4cccc(-c5ccc6c(c5)-c5cccc7cccc(c57)O6)c4)c4ccccc4-c4cccc5oc6ccccc6c45)cc3)cc2-c2ccccc2)cc1 ; c1ccc(-c2ccc(-c3ccc(N(c4cccc(-c5ccc6c(c5)-c5cccc7cccc(c57)S6)c4)c4cccc5sc6ccccc6c45)cc3)cc2-c2ccccc2)cc1]

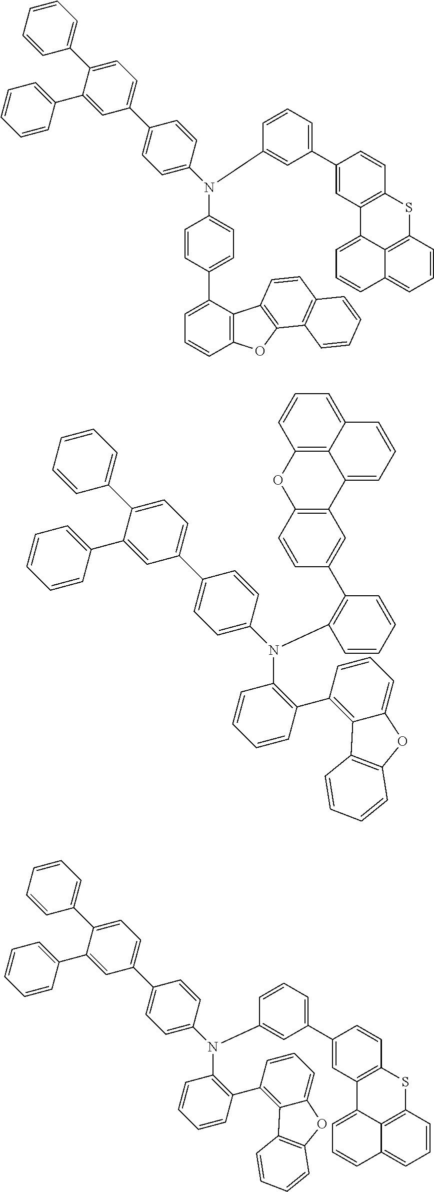 [SMILES: c1ccc(-c2ccc(-c3ccc(N(c4ccc(-c5cccc6oc7c8ccccc8ccc7c56)cc4)c4cccc(-c5ccc6c(c5)-c5cccc7cccc(c57)S6)c4)cc3)cc2-c2ccccc2)cc1 ; c1ccc(-c2ccc(-c3ccc(N(c4ccccc4-c4ccc5c(c4)-c4cccc6cccc(c46)O5)c4ccccc4-c4cccc5oc6ccccc6c45)cc3)cc2-c2ccccc2)cc1 ; c1ccc(-c2ccc(-c3ccc(N(c4cccc(-c5ccc6c(c5)-c5cccc7cccc(c57)S6)c4)c4ccccc4-c4cccc5oc6ccccc6c45)cc3)cc2-c2ccccc2)cc1]

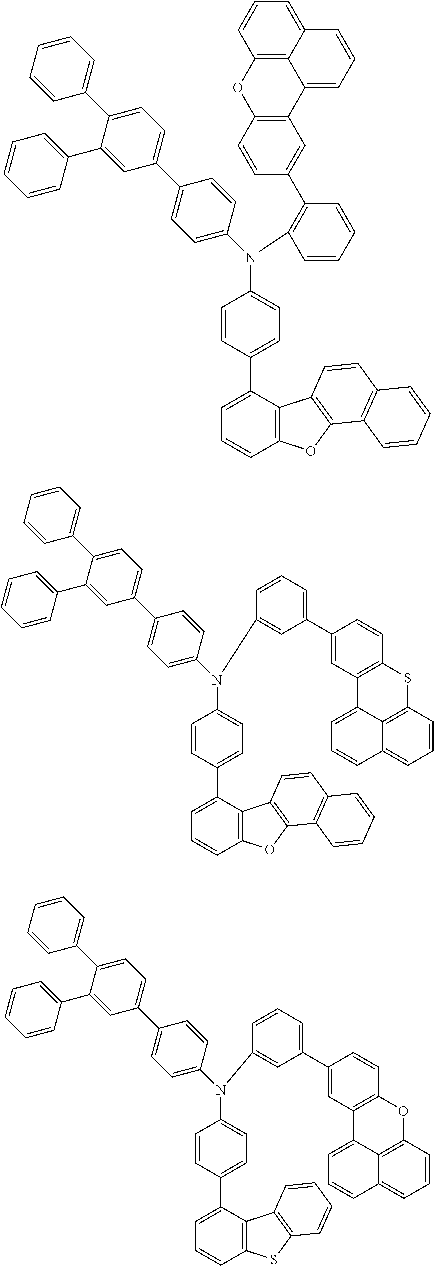 [SMILES: c1ccc(-c2ccc(-c3ccc(N(c4ccc(-c5cccc6oc7c8ccccc8ccc7c56)cc4)c4ccccc4-c4ccc5c(c4)-c4cccc6cccc(c46)O5)cc3)cc2-c2ccccc2)cc1 ; c1ccc(-c2ccc(-c3ccc(N(c4ccc(-c5cccc6oc7c8ccccc8ccc7c56)cc4)c4cccc(-c5ccc6c(c5)-c5cccc7cccc(c57)S6)c4)cc3)cc2-c2ccccc2)cc1 ; c1ccc(-c2ccc(-c3ccc(N(c4ccc(-c5cccc6sc7ccccc7c56)cc4)c4cccc(-c5ccc6c(c5)-c5cccc7cccc(c57)O6)c4)cc3)cc2-c2ccccc2)cc1]

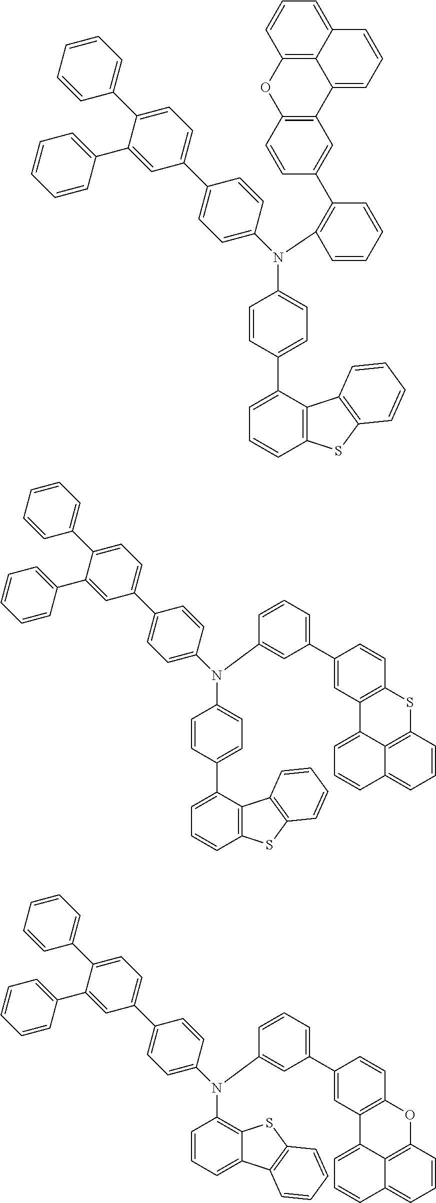 [SMILES: c1ccc(-c2ccc(-c3ccc(N(c4ccc(-c5cccc6sc7ccccc7c56)cc4)c4ccccc4-c4ccc5c(c4)-c4cccc6cccc(c46)O5)cc3)cc2-c2ccccc2)cc1 ; c1ccc(-c2ccc(-c3ccc(N(c4ccc(-c5cccc6sc7ccccc7c56)cc4)c4cccc(-c5ccc6c(c5)-c5cccc7cccc(c57)S6)c4)cc3)cc2-c2ccccc2)cc1 ; c1ccc(-c2ccc(-c3ccc(N(c4cccc(-c5ccc6c(c5)-c5cccc7cccc(c57)O6)c4)c4cccc5c4sc4ccccc45)cc3)cc2-c2ccccc2)cc1]

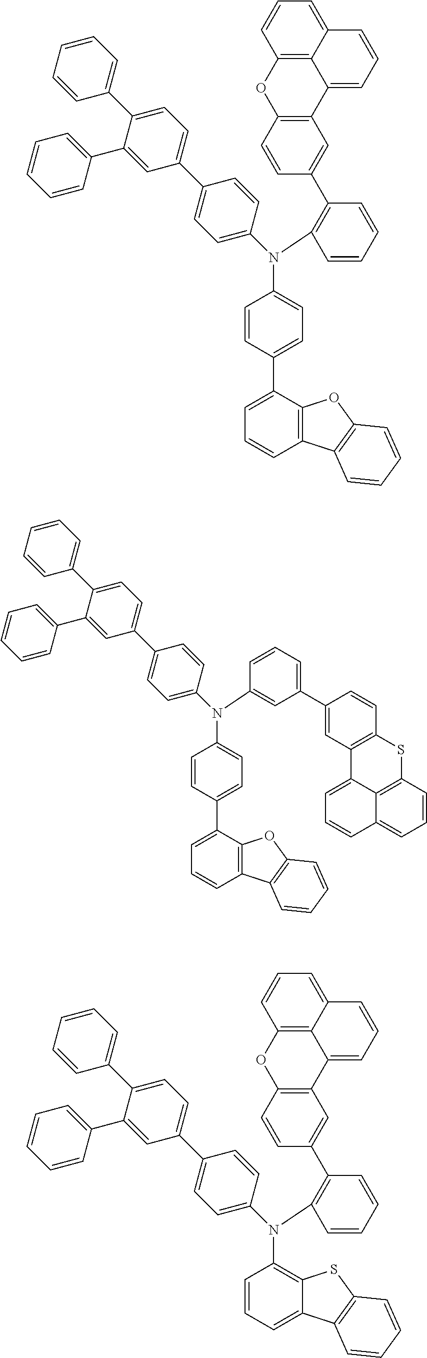 [SMILES: c1ccc(-c2ccc(-c3ccc(N(c4ccc(-c5cccc6c5oc5ccccc56)cc4)c4ccccc4-c4ccc5c(c4)-c4cccc6cccc(c46)O5)cc3)cc2-c2ccccc2)cc1 ; c1ccc(-c2ccc(-c3ccc(N(c4ccc(-c5cccc6c5oc5ccccc56)cc4)c4cccc(-c5ccc6c(c5)-c5cccc7cccc(c57)S6)c4)cc3)cc2-c2ccccc2)cc1 ; c1ccc(-c2ccc(-c3ccc(N(c4ccccc4-c4ccc5c(c4)-c4cccc6cccc(c46)O5)c4cccc5c4sc4ccccc45)cc3)cc2-c2ccccc2)cc1]

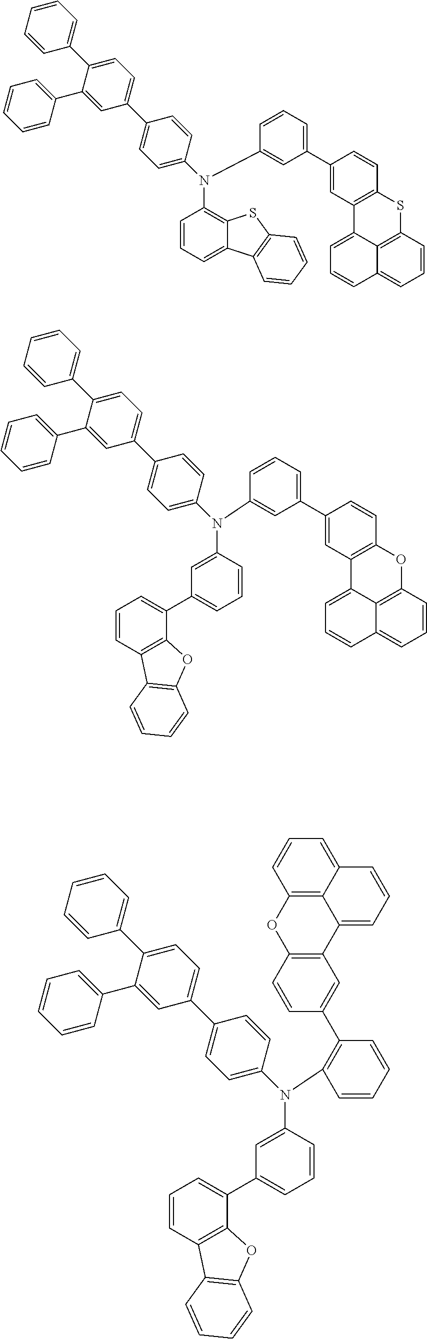 [SMILES: c1ccc(-c2ccc(-c3ccc(N(c4cccc(-c5ccc6c(c5)-c5cccc7cccc(c57)S6)c4)c4cccc5c4sc4ccccc45)cc3)cc2-c2ccccc2)cc1 ; c1ccc(-c2ccc(-c3ccc(N(c4cccc(-c5ccc6c(c5)-c5cccc7cccc(c57)O6)c4)c4cccc(-c5cccc6c5oc5ccccc56)c4)cc3)cc2-c2ccccc2)cc1 ; c1ccc(-c2ccc(-c3ccc(N(c4cccc(-c5cccc6c5oc5ccccc56)c4)c4ccccc4-c4ccc5c(c4)-c4cccc6cccc(c46)O5)cc3)cc2-c2ccccc2)cc1]

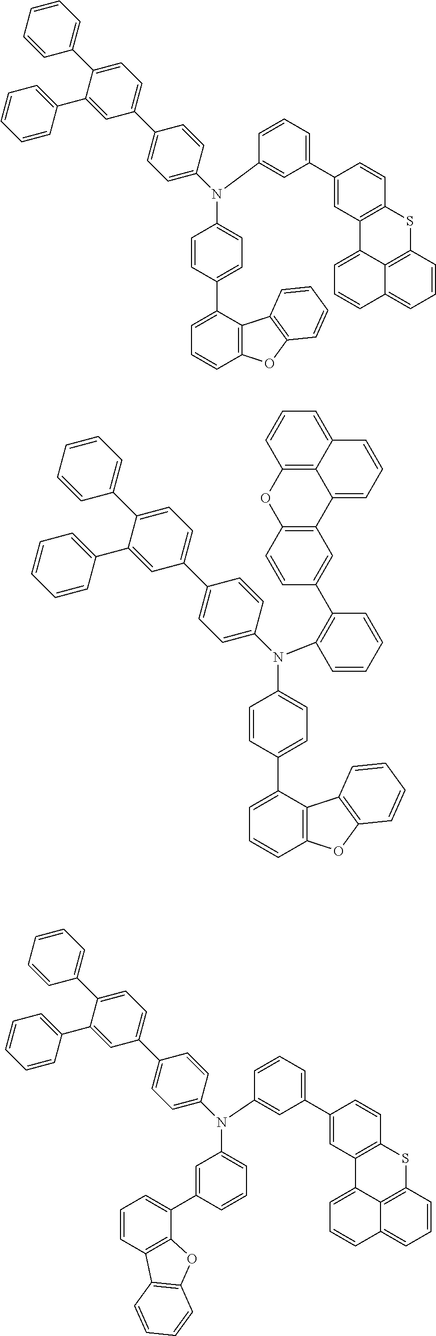 [SMILES: c1ccc(-c2ccc(-c3ccc(N(c4ccc(-c5cccc6oc7ccccc7c56)cc4)c4cccc(-c5ccc6c(c5)-c5cccc7cccc(c57)S6)c4)cc3)cc2-c2ccccc2)cc1 ; c1ccc(-c2ccc(-c3ccc(N(c4ccc(-c5cccc6oc7ccccc7c56)cc4)c4ccccc4-c4ccc5c(c4)-c4cccc6cccc(c46)O5)cc3)cc2-c2ccccc2)cc1 ; c1ccc(-c2ccc(-c3ccc(N(c4cccc(-c5ccc6c(c5)-c5cccc7cccc(c57)S6)c4)c4cccc(-c5cccc6c5oc5ccccc56)c4)cc3)cc2-c2ccccc2)cc1]

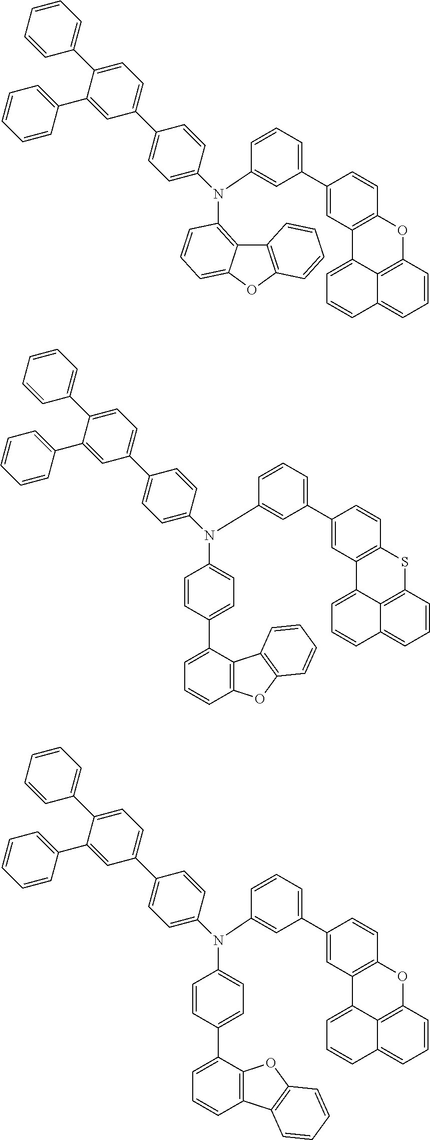 [SMILES: c1ccc(-c2ccc(-c3ccc(N(c4cccc(-c5ccc6c(c5)-c5cccc7cccc(c57)O6)c4)c4cccc5oc6ccccc6c45)cc3)cc2-c2ccccc2)cc1 ; c1ccc(-c2ccc(-c3ccc(N(c4ccc(-c5cccc6oc7ccccc7c56)cc4)c4cccc(-c5ccc6c(c5)-c5cccc7cccc(c57)S6)c4)cc3)cc2-c2ccccc2)cc1 ; c1ccc(-c2ccc(-c3ccc(N(c4ccc(-c5cccc6c5oc5ccccc56)cc4)c4cccc(-c5ccc6c(c5)-c5cccc7cccc(c57)O6)c4)cc3)cc2-c2ccccc2)cc1]

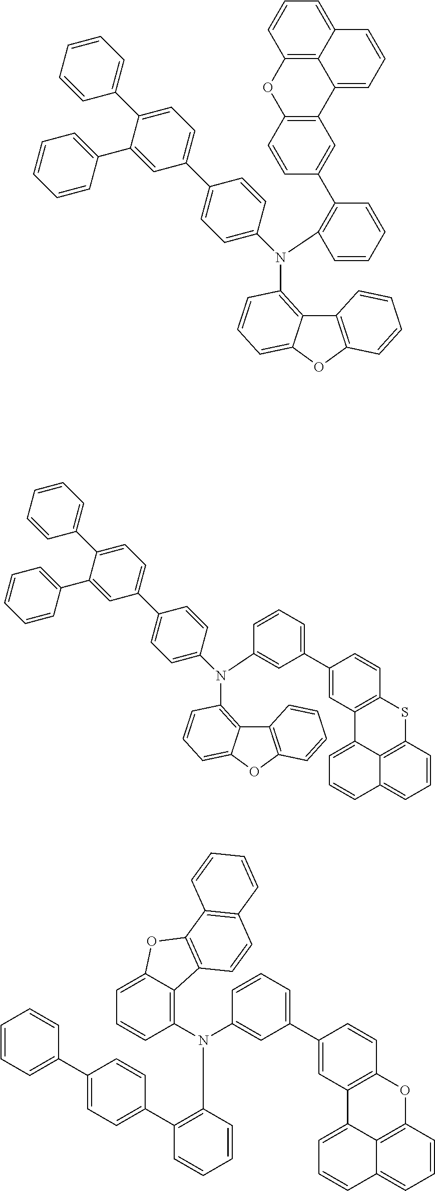 [SMILES: c1ccc(-c2ccc(-c3ccc(N(c4ccccc4-c4ccc5c(c4)-c4cccc6cccc(c46)O5)c4cccc5oc6ccccc6c45)cc3)cc2-c2ccccc2)cc1 ; c1ccc(-c2ccc(-c3ccc(N(c4cccc(-c5ccc6c(c5)-c5cccc7cccc(c57)S6)c4)c4cccc5oc6ccccc6c45)cc3)cc2-c2ccccc2)cc1 ; c1ccc(-c2ccc(-c3ccccc3N(c3cccc(-c4ccc5c(c4)-c4cccc6cccc(c46)O5)c3)c3cccc4oc5c6ccccc6ccc5c34)cc2)cc1]

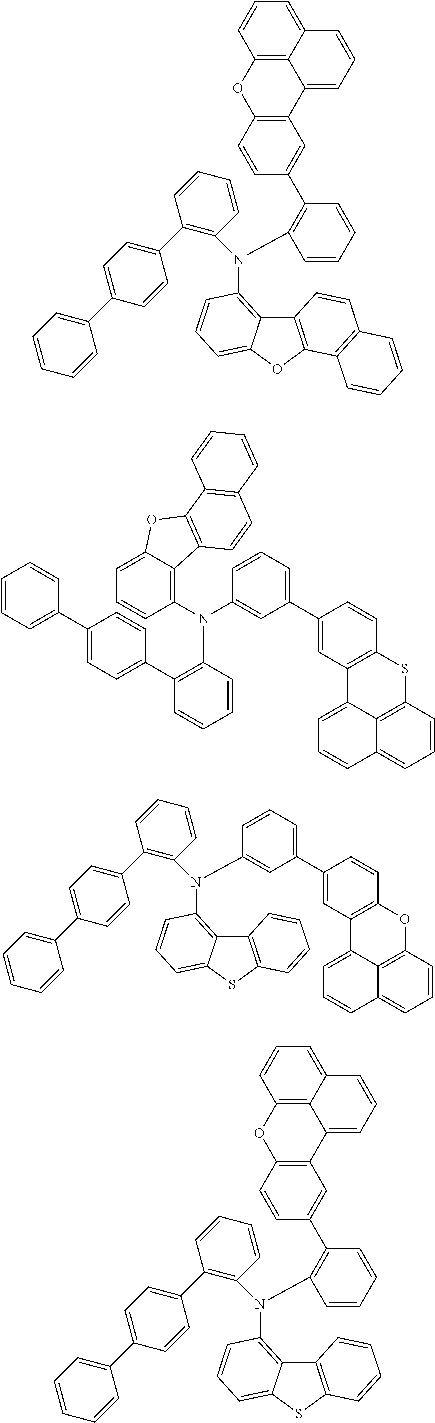 [SMILES: c1ccc(-c2ccc(-c3ccccc3N(c3ccccc3-c3ccc4c(c3)-c3cccc5cccc(c35)O4)c3cccc4oc5c6ccccc6ccc5c34)cc2)cc1 ; c1ccc(-c2ccc(-c3ccccc3N(c3cccc(-c4ccc5c(c4)-c4cccc6cccc(c46)S5)c3)c3cccc4oc5c6ccccc6ccc5c34)cc2)cc1 ; c1ccc(-c2ccc(-c3ccccc3N(c3cccc(-c4ccc5c(c4)-c4cccc6cccc(c46)O5)c3)c3cccc4sc5ccccc5c34)cc2)cc1 ; c1ccc(-c2ccc(-c3ccccc3N(c3ccccc3-c3ccc4c(c3)-c3cccc5cccc(c35)O4)c3cccc4sc5ccccc5c34)cc2)cc1]

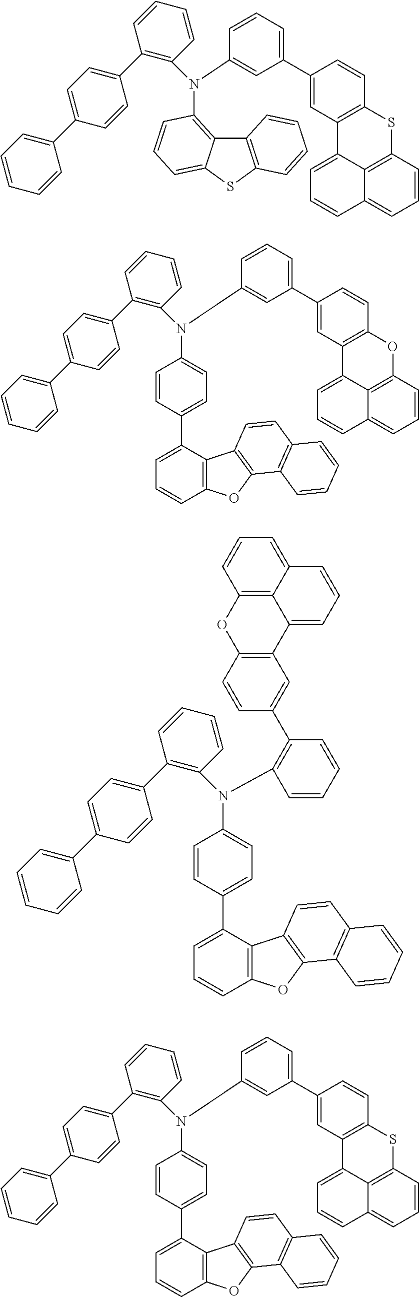

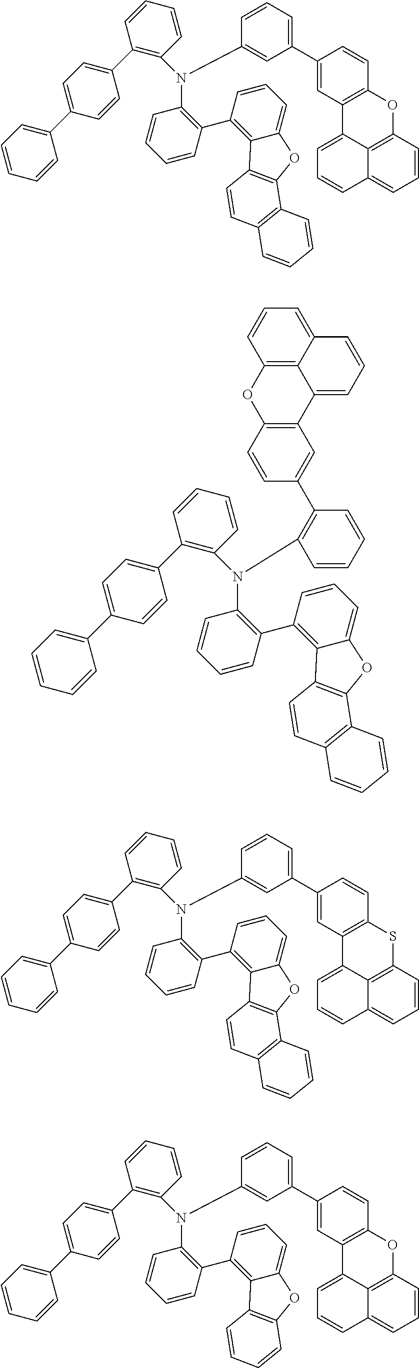 [SMILES: c1ccc(-c2ccc(-c3ccccc3N(c3cccc(-c4ccc5c(c4)-c4cccc6cccc(c46)O5)c3)c3ccccc3-c3cccc4oc5c6ccccc6ccc5c34)cc2)cc1 ; c1ccc(-c2ccc(-c3ccccc3N(c3ccccc3-c3ccc4c(c3)-c3cccc5cccc(c35)O4)c3ccccc3-c3cccc4oc5c6ccccc6ccc5c34)cc2)cc1 ; c1ccc(-c2ccc(-c3ccccc3N(c3cccc(-c4ccc5c(c4)-c4cccc6cccc(c46)S5)c3)c3ccccc3-c3cccc4oc5c6ccccc6ccc5c34)cc2)cc1 ; c1ccc(-c2ccc(-c3ccccc3N(c3cccc(-c4ccc5c(c4)-c4cccc6cccc(c46)O5)c3)c3ccccc3-c3cccc4oc5ccccc5c34)cc2)cc1]

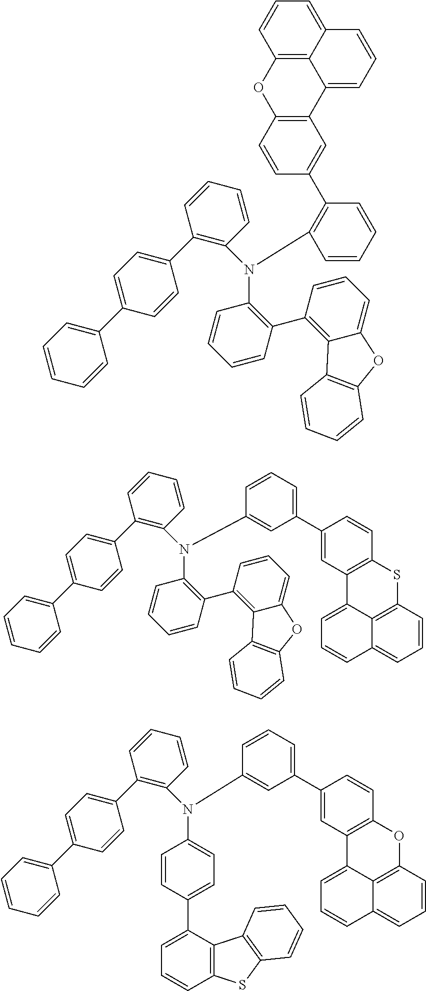

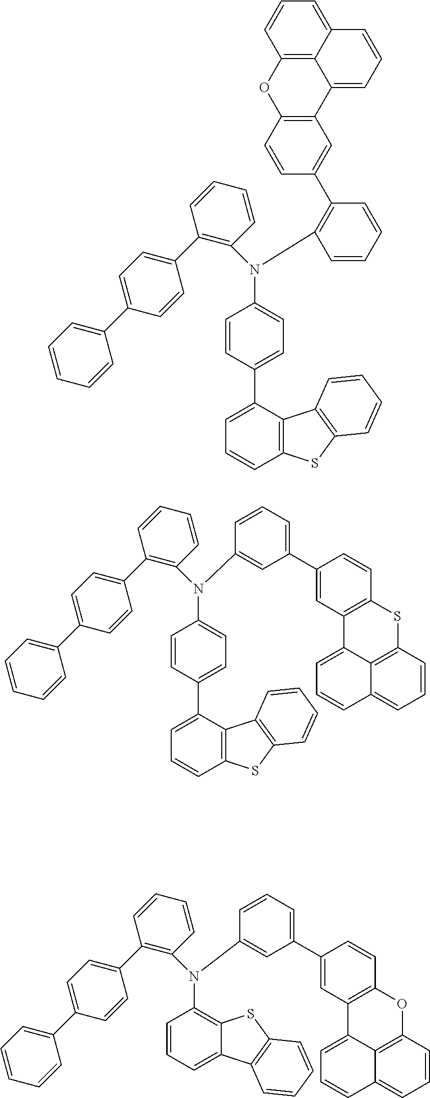

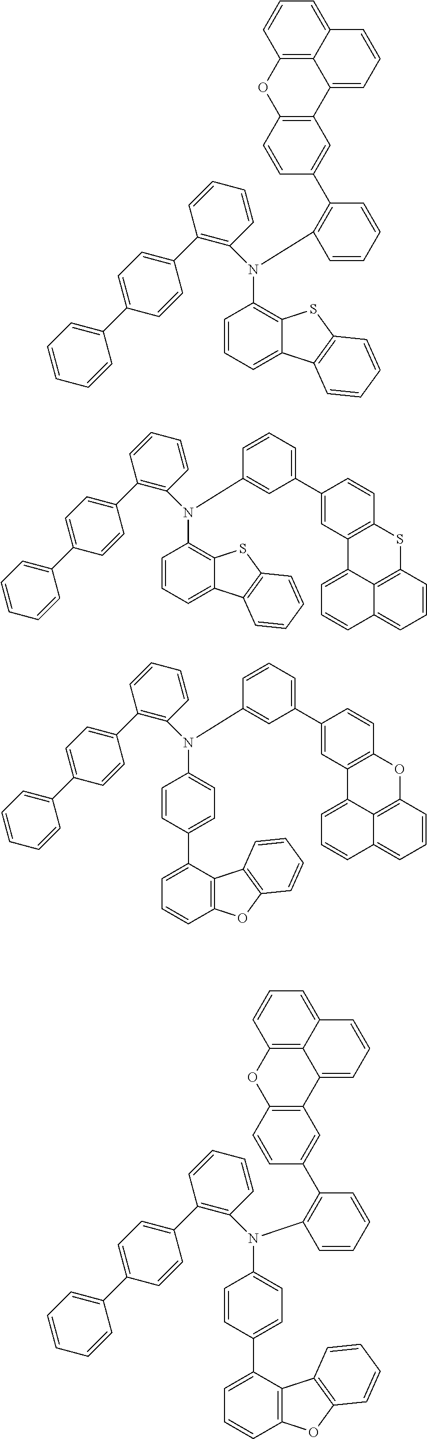

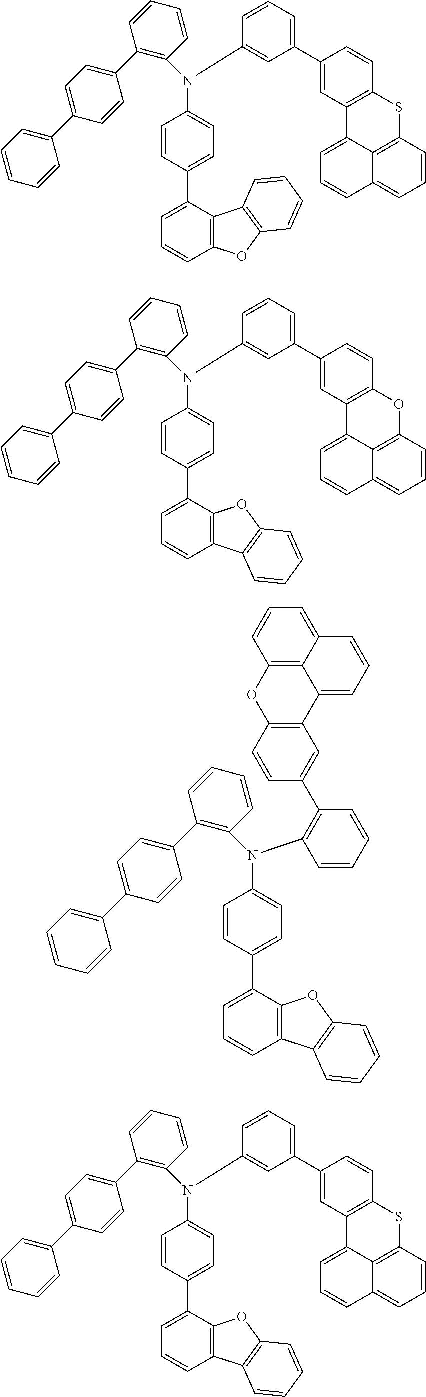

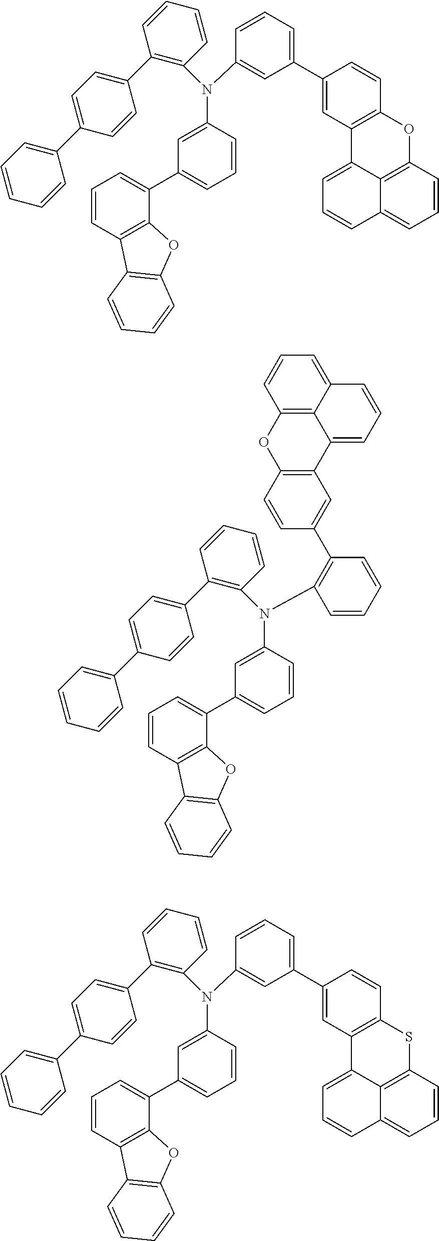 [SMILES: c1ccc(-c2ccc(-c3ccccc3N(c3cccc(-c4ccc5c(c4)-c4cccc6cccc(c46)O5)c3)c3cccc(-c4cccc5c4oc4ccccc45)c3)cc2)cc1 ; c1ccc(-c2ccc(-c3ccccc3N(c3cccc(-c4cccc5c4oc4ccccc45)c3)c3ccccc3-c3ccc4c(c3)-c3cccc5cccc(c35)O4)cc2)cc1 ; c1ccc(-c2ccc(-c3ccccc3N(c3cccc(-c4ccc5c(c4)-c4cccc6cccc(c46)S5)c3)c3cccc(-c4cccc5c4oc4ccccc45)c3)cc2)cc1]

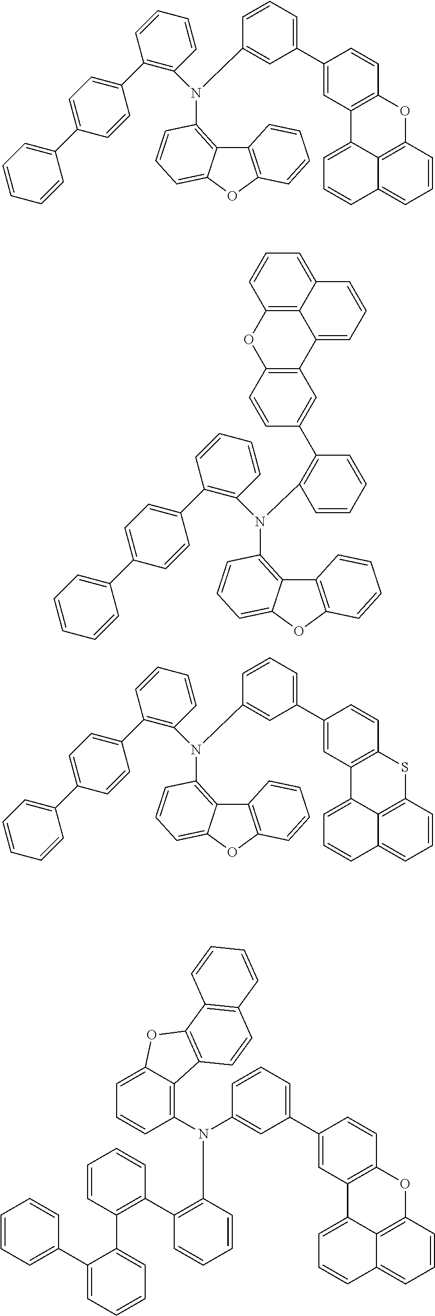

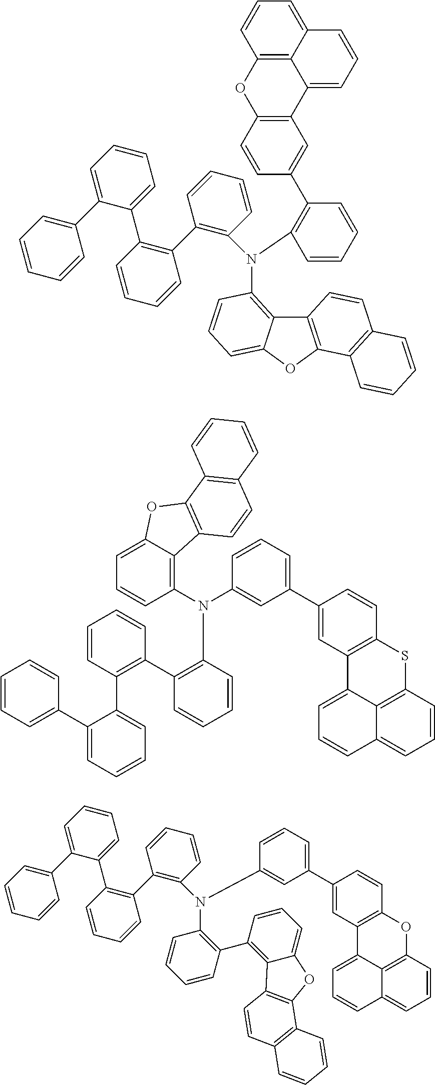

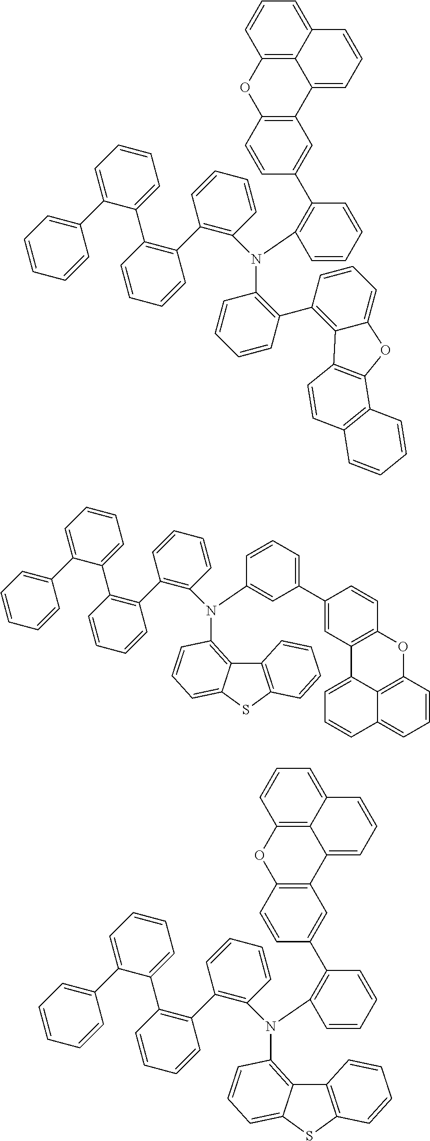 [SMILES: c1ccc(-c2ccccc2-c2ccccc2-c2ccccc2N(c2ccccc2-c2ccc3c(c2)-c2cccc4cccc(c24)O3)c2ccccc2-c2cccc3oc4c5ccccc5ccc4c23)cc1 ; c1ccc(-c2ccccc2-c2ccccc2-c2ccccc2N(c2cccc(-c3ccc4c(c3)-c3cccc5cccc(c35)O4)c2)c2cccc3sc4ccccc4c23)cc1 ; c1ccc(-c2ccccc2-c2ccccc2-c2ccccc2N(c2ccccc2-c2ccc3c(c2)-c2cccc4cccc(c24)O3)c2cccc3sc4ccccc4c23)cc1]

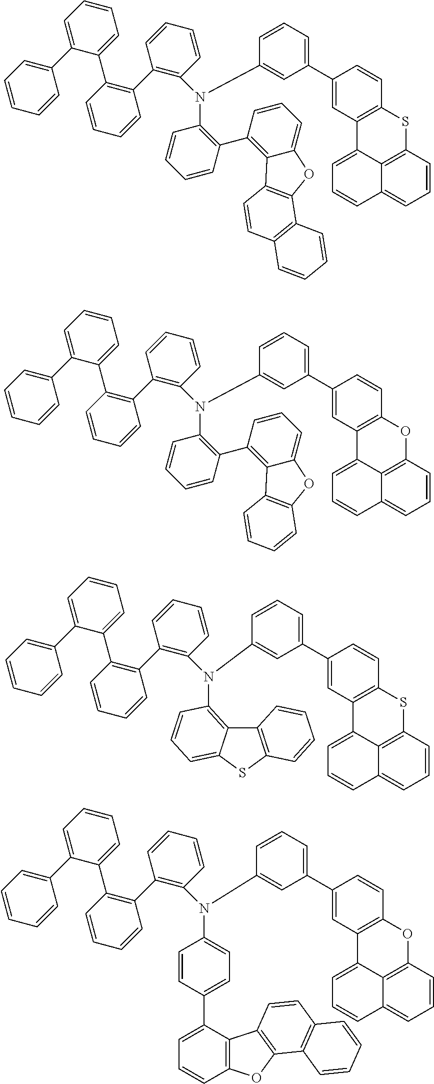 [SMILES: c1ccc(-c2ccccc2-c2ccccc2-c2ccccc2N(c2cccc(-c3ccc4c(c3)-c3cccc5cccc(c35)S4)c2)c2ccccc2-c2cccc3oc4c5ccccc5ccc4c23)cc1 ; c1ccc(-c2ccccc2-c2ccccc2-c2ccccc2N(c2cccc(-c3ccc4c(c3)-c3cccc5cccc(c35)O4)c2)c2ccccc2-c2cccc3oc4ccccc4c23)cc1 ; c1ccc(-c2ccccc2-c2ccccc2-c2ccccc2N(c2cccc(-c3ccc4c(c3)-c3cccc5cccc(c35)S4)c2)c2cccc3sc4ccccc4c23)cc1 ; c1ccc(-c2ccccc2-c2ccccc2-c2ccccc2N(c2ccc(-c3cccc4oc5c6ccccc6ccc5c34)cc2)c2cccc(-c3ccc4c(c3)-c3cccc5cccc(c35)O4)c2)cc1]

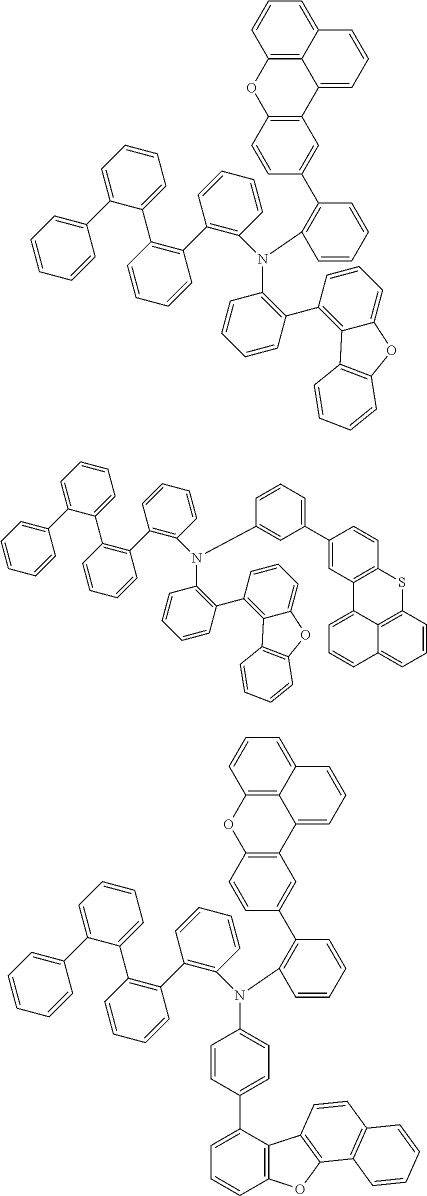

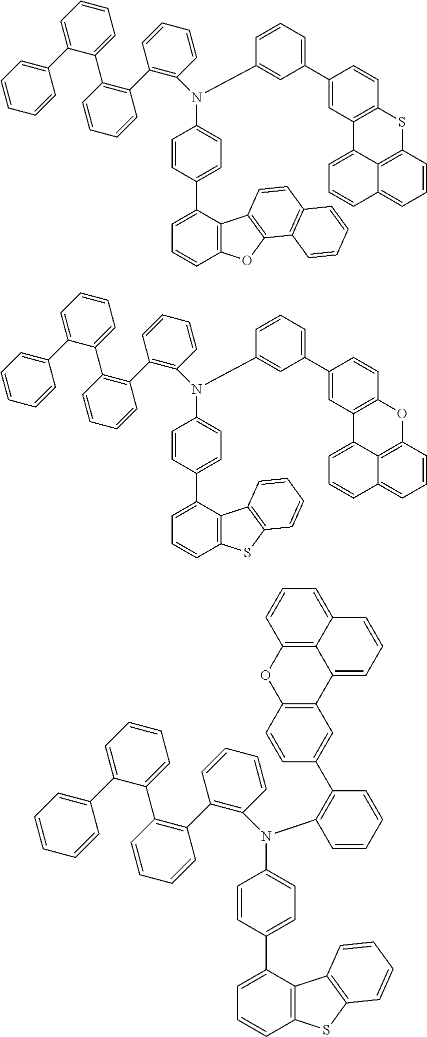 [SMILES: c1ccc(-c2ccccc2-c2ccccc2-c2ccccc2N(c2ccc(-c3cccc4oc5c6ccccc6ccc5c34)cc2)c2cccc(-c3ccc4c(c3)-c3cccc5cccc(c35)S4)c2)cc1 ; c1ccc(-c2ccccc2-c2ccccc2-c2ccccc2N(c2ccc(-c3cccc4sc5ccccc5c34)cc2)c2cccc(-c3ccc4c(c3)-c3cccc5cccc(c35)O4)c2)cc1 ; c1ccc(-c2ccccc2-c2ccccc2-c2ccccc2N(c2ccc(-c3cccc4sc5ccccc5c34)cc2)c2ccccc2-c2ccc3c(c2)-c2cccc4cccc(c24)O3)cc1]

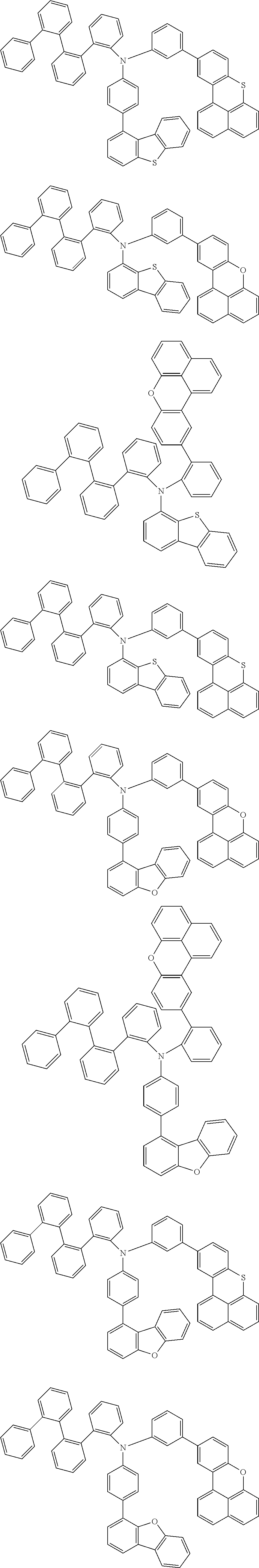

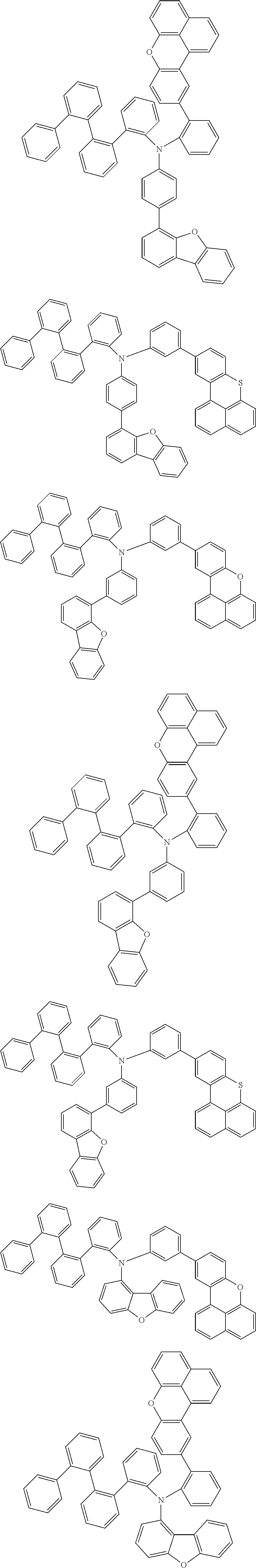

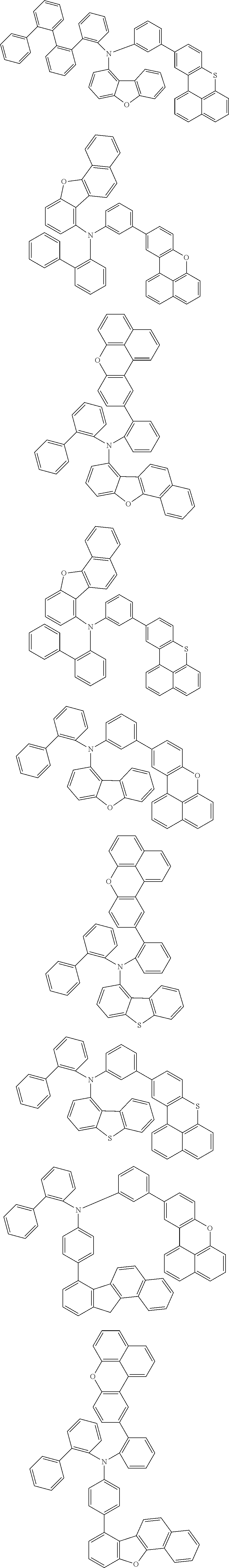

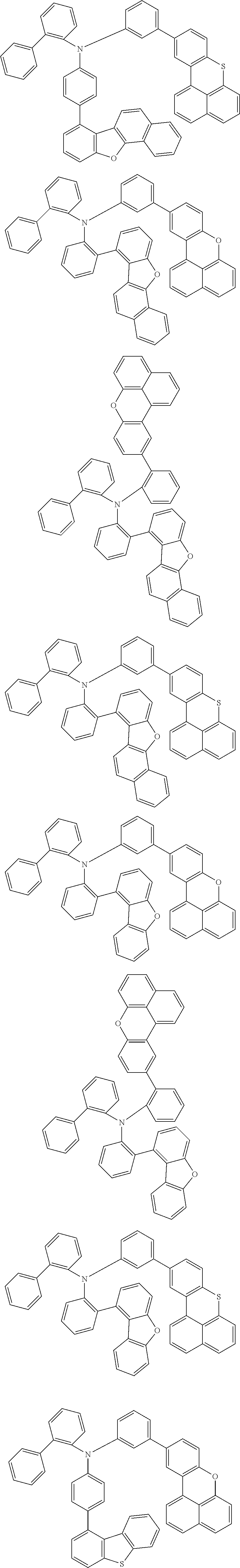

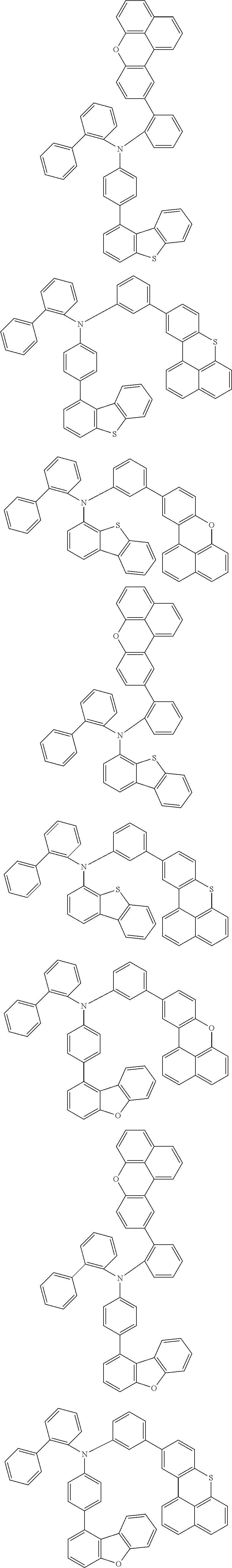

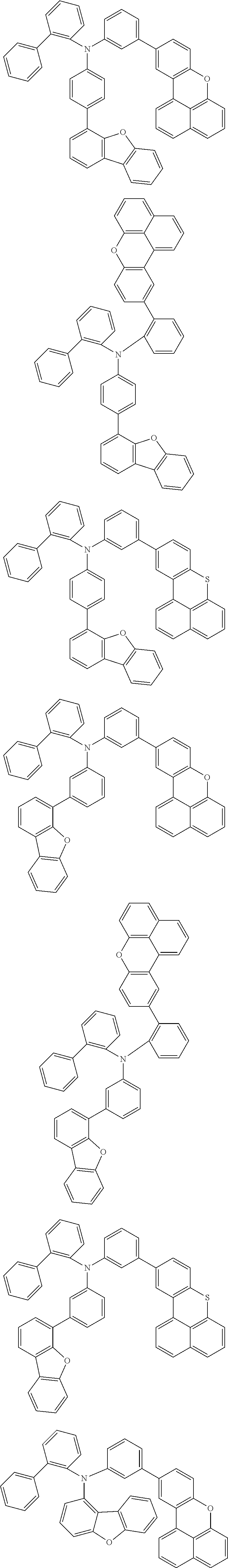

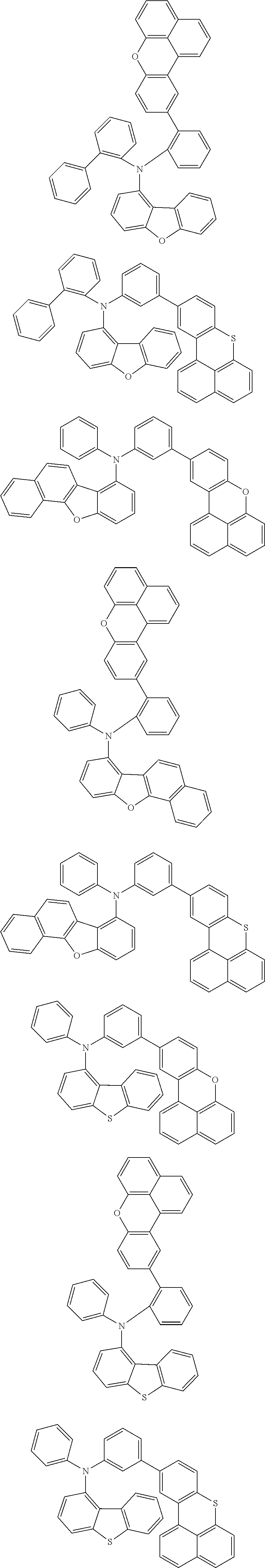

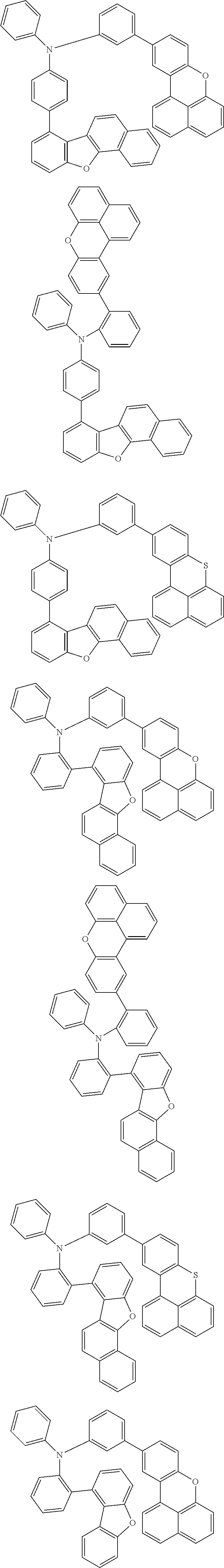

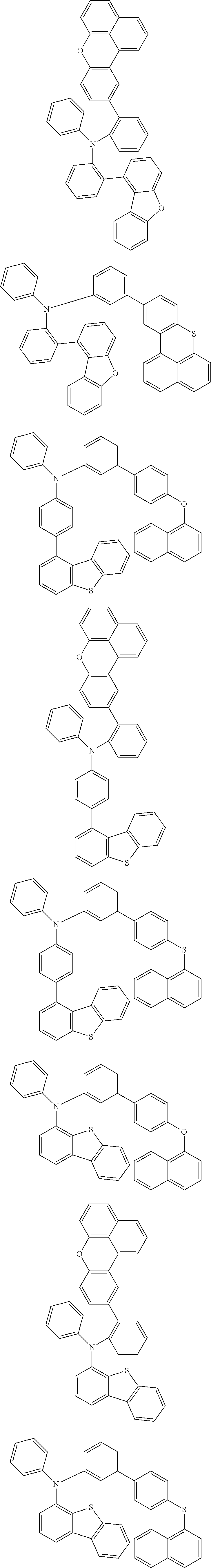

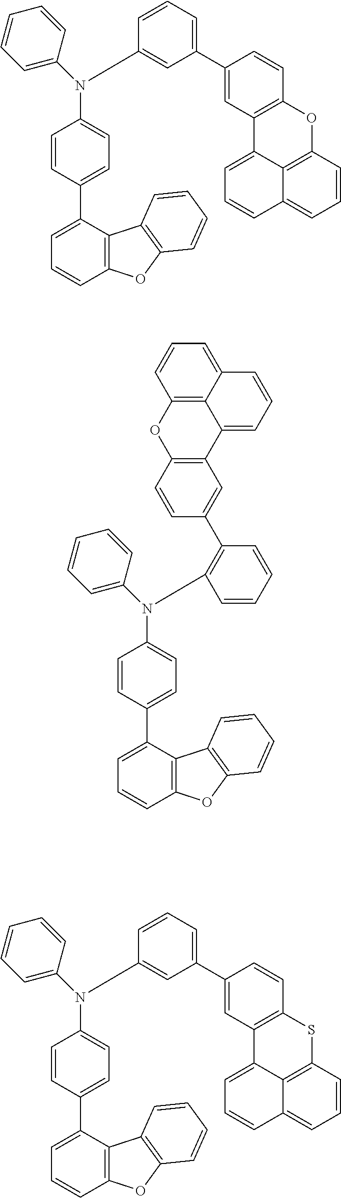 [SMILES: c1ccc(N(c2ccc(-c3cccc4oc5ccccc5c34)cc2)c2cccc(-c3ccc4c(c3)-c3cccc5cccc(c35)O4)c2)cc1 ; c1ccc(N(c2ccc(-c3cccc4oc5ccccc5c34)cc2)c2ccccc2-c2ccc3c(c2)-c2cccc4cccc(c24)O3)cc1 ; c1ccc(N(c2ccc(-c3cccc4oc5ccccc5c34)cc2)c2cccc(-c3ccc4c(c3)-c3cccc5cccc(c35)S4)c2)cc1]

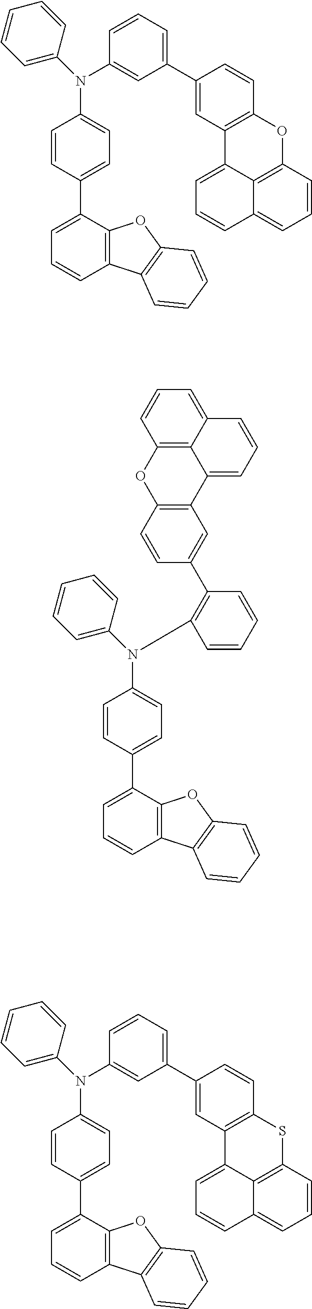 [SMILES: c1ccc(N(c2ccc(-c3cccc4c3oc3ccccc34)cc2)c2cccc(-c3ccc4c(c3)-c3cccc5cccc(c35)O4)c2)cc1 ; c1ccc(N(c2ccc(-c3cccc4c3oc3ccccc34)cc2)c2ccccc2-c2ccc3c(c2)-c2cccc4cccc(c24)O3)cc1 ; c1ccc(N(c2ccc(-c3cccc4c3oc3ccccc34)cc2)c2cccc(-c3ccc4c(c3)-c3cccc5cccc(c35)S4)c2)cc1]

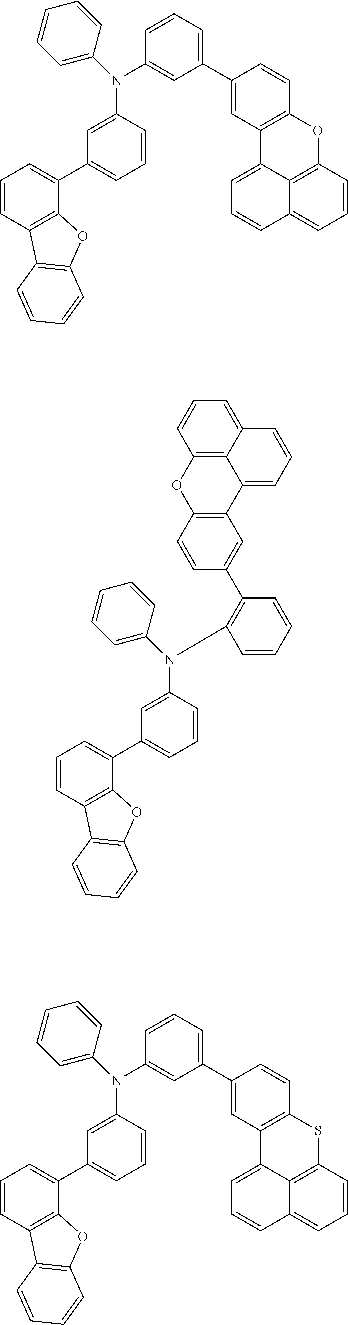 [SMILES: c1ccc(N(c2cccc(-c3ccc4c(c3)-c3cccc5cccc(c35)O4)c2)c2cccc(-c3cccc4c3oc3ccccc34)c2)cc1 ; c1ccc(N(c2cccc(-c3cccc4c3oc3ccccc34)c2)c2ccccc2-c2ccc3c(c2)-c2cccc4cccc(c24)O3)cc1 ; c1ccc(N(c2cccc(-c3ccc4c(c3)-c3cccc5cccc(c35)S4)c2)c2cccc(-c3cccc4c3oc3ccccc34)c2)cc1]

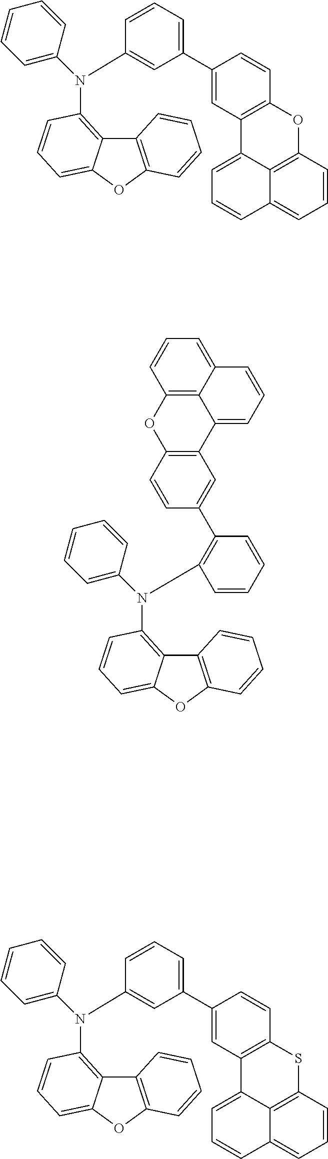 [SMILES: c1ccc(N(c2cccc(-c3ccc4c(c3)-c3cccc5cccc(c35)O4)c2)c2cccc3oc4ccccc4c23)cc1 ; c1ccc(N(c2ccccc2-c2ccc3c(c2)-c2cccc4cccc(c24)O3)c2cccc3oc4ccccc4c23)cc1 ; c1ccc(N(c2cccc(-c3ccc4c(c3)-c3cccc5cccc(c35)S4)c2)c2cccc3oc4ccccc4c23)cc1]

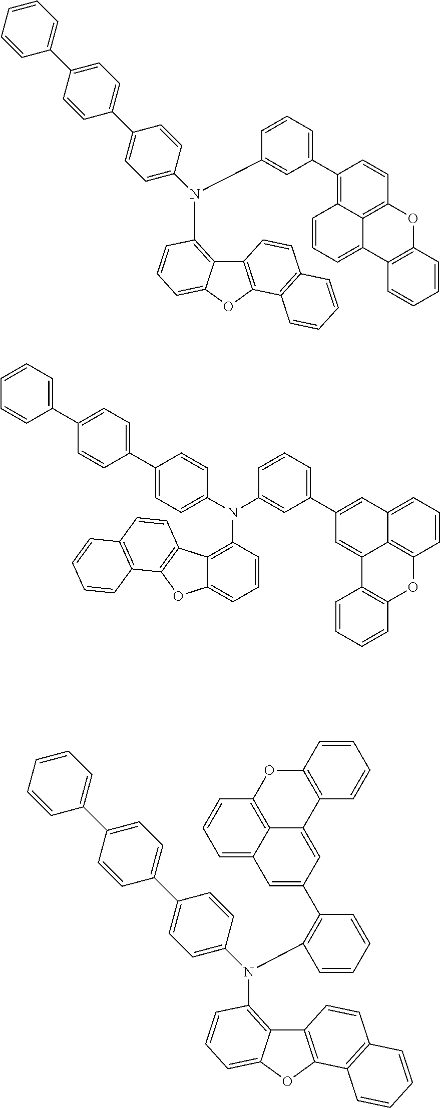 [SMILES: c1ccc(-c2ccc(-c3ccc(N(c4cccc(-c5ccc6c7c(cccc57)-c5ccccc5O6)c4)c4cccc5oc6c7ccccc7ccc6c45)cc3)cc2)cc1 ; c1ccc(-c2ccc(-c3ccc(N(c4cccc(-c5cc6c7c(cccc7c5)Oc5ccccc5-6)c4)c4cccc5oc6c7ccccc7ccc6c45)cc3)cc2)cc1 ; c1ccc(-c2ccc(-c3ccc(N(c4ccccc4-c4cc5c6c(cccc6c4)Oc4ccccc4-5)c4cccc5oc6c7ccccc7ccc6c45)cc3)cc2)cc1]

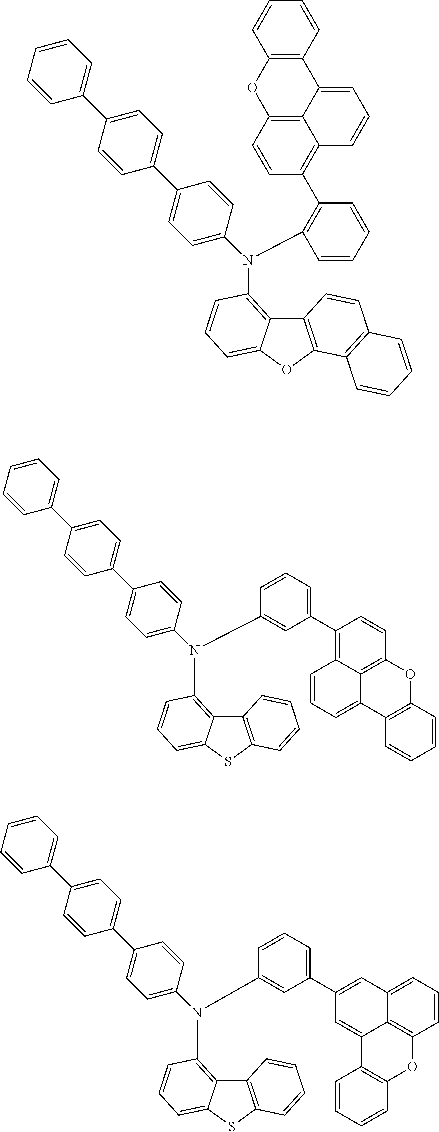 [SMILES: c1ccc(-c2ccc(-c3ccc(N(c4ccccc4-c4ccc5c6c(cccc46)-c4ccccc4O5)c4cccc5oc6c7ccccc7ccc6c45)cc3)cc2)cc1 ; c1ccc(-c2ccc(-c3ccc(N(c4cccc(-c5ccc6c7c(cccc57)-c5ccccc5O6)c4)c4cccc5sc6ccccc6c45)cc3)cc2)cc1 ; c1ccc(-c2ccc(-c3ccc(N(c4cccc(-c5cc6c7c(cccc7c5)Oc5ccccc5-6)c4)c4cccc5sc6ccccc6c45)cc3)cc2)cc1]

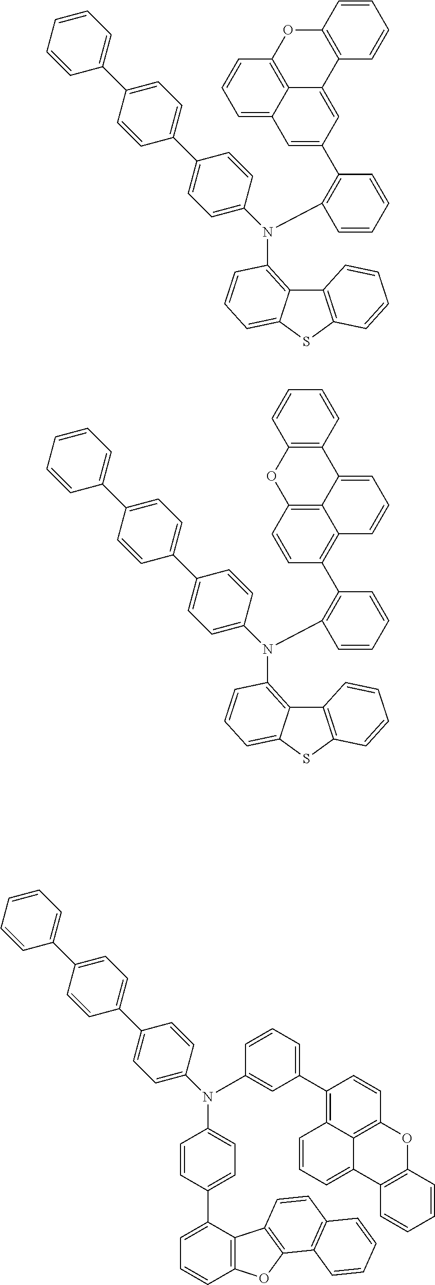 [SMILES: c1ccc(-c2ccc(-c3ccc(N(c4ccccc4-c4cc5c6c(cccc6c4)Oc4ccccc4-5)c4cccc5sc6ccccc6c45)cc3)cc2)cc1 ; c1ccc(-c2ccc(-c3ccc(N(c4ccccc4-c4ccc5c6c(cccc46)-c4ccccc4O5)c4cccc5sc6ccccc6c45)cc3)cc2)cc1 ; c1ccc(-c2ccc(-c3ccc(N(c4ccc(-c5cccc6oc7c8ccccc8ccc7c56)cc4)c4cccc(-c5ccc6c7c(cccc57)-c5ccccc5O6)c4)cc3)cc2)cc1]

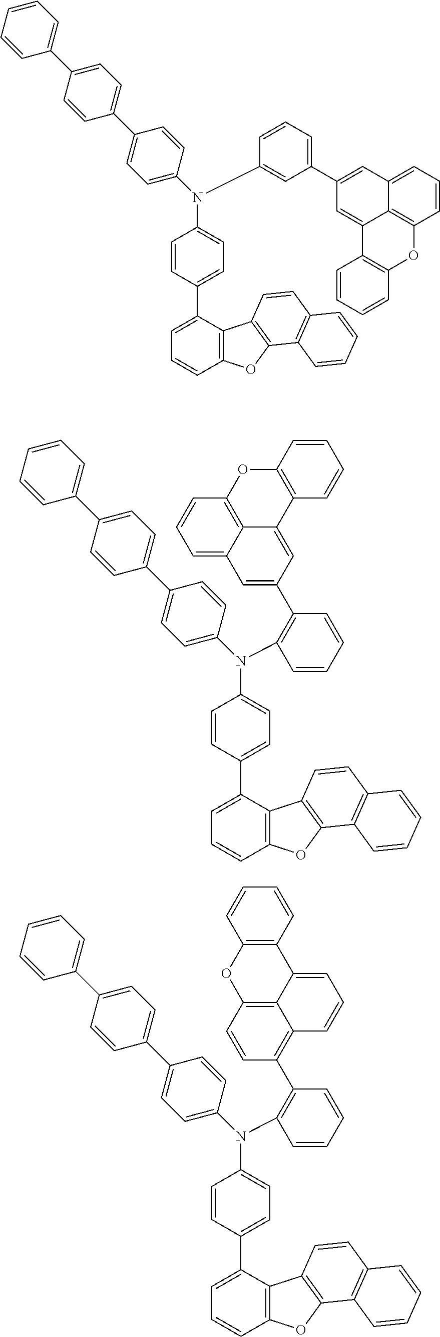

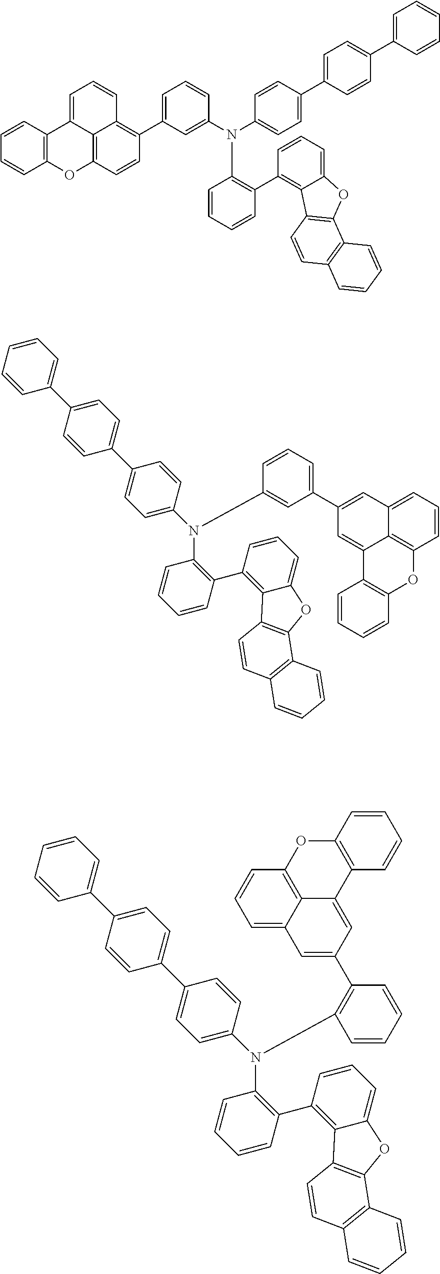 [SMILES: c1ccc(-c2ccc(-c3ccc(N(c4cccc(-c5ccc6c7c(cccc57)-c5ccccc5O6)c4)c4ccccc4-c4cccc5oc6c7ccccc7ccc6c45)cc3)cc2)cc1 ; c1ccc(-c2ccc(-c3ccc(N(c4cccc(-c5cc6c7c(cccc7c5)Oc5ccccc5-6)c4)c4ccccc4-c4cccc5oc6c7ccccc7ccc6c45)cc3)cc2)cc1 ; c1ccc(-c2ccc(-c3ccc(N(c4ccccc4-c4cc5c6c(cccc6c4)Oc4ccccc4-5)c4ccccc4-c4cccc5oc6c7ccccc7ccc6c45)cc3)cc2)cc1]

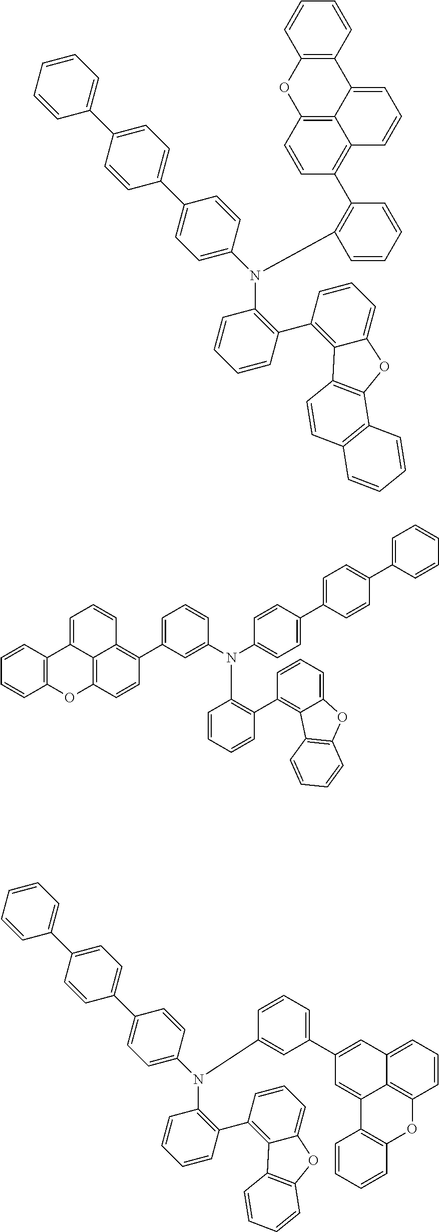 [SMILES: c1ccc(-c2ccc(-c3ccc(N(c4ccccc4-c4ccc5c6c(cccc46)-c4ccccc4O5)c4ccccc4-c4cccc5oc6c7ccccc7ccc6c45)cc3)cc2)cc1 ; c1ccc(-c2ccc(-c3ccc(N(c4cccc(-c5ccc6c7c(cccc57)-c5ccccc5O6)c4)c4ccccc4-c4cccc5oc6ccccc6c45)cc3)cc2)cc1 ; c1ccc(-c2ccc(-c3ccc(N(c4cccc(-c5cc6c7c(cccc7c5)Oc5ccccc5-6)c4)c4ccccc4-c4cccc5oc6ccccc6c45)cc3)cc2)cc1]

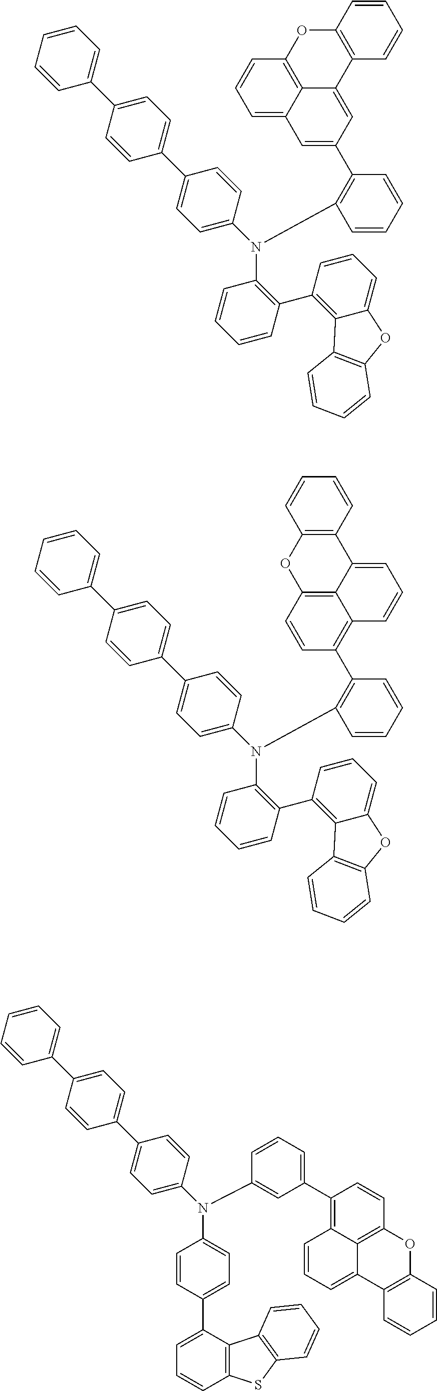 [SMILES: c1ccc(-c2ccc(-c3ccc(N(c4ccccc4-c4cc5c6c(cccc6c4)Oc4ccccc4-5)c4ccccc4-c4cccc5oc6ccccc6c45)cc3)cc2)cc1 ; c1ccc(-c2ccc(-c3ccc(N(c4ccccc4-c4ccc5c6c(cccc46)-c4ccccc4O5)c4ccccc4-c4cccc5oc6ccccc6c45)cc3)cc2)cc1 ; c1ccc(-c2ccc(-c3ccc(N(c4ccc(-c5cccc6sc7ccccc7c56)cc4)c4cccc(-c5ccc6c7c(cccc57)-c5ccccc5O6)c4)cc3)cc2)cc1]

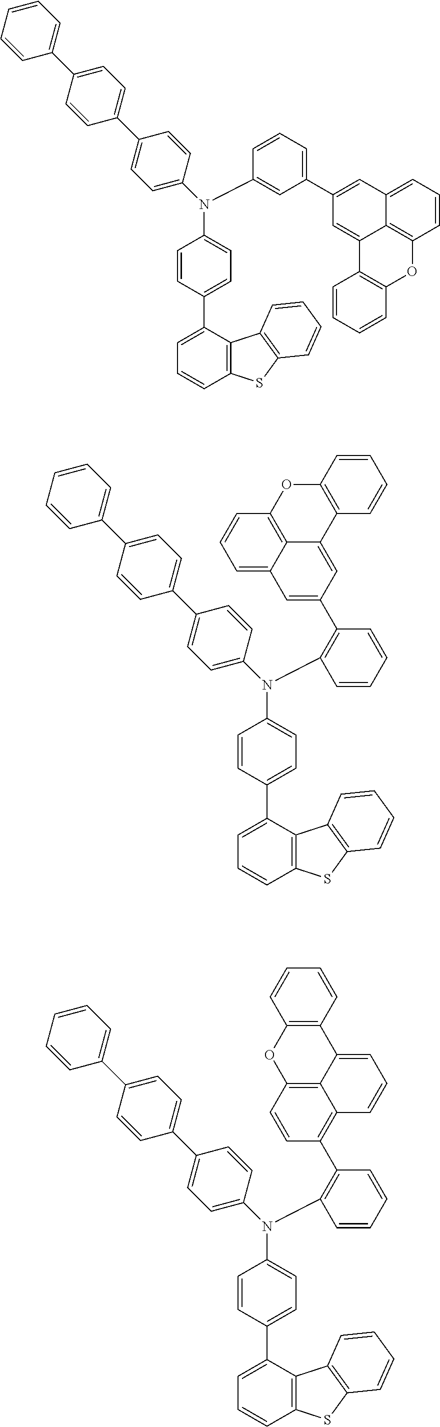 [SMILES: c1ccc(-c2ccc(-c3ccc(N(c4ccc(-c5cccc6sc7ccccc7c56)cc4)c4cccc(-c5cc6c7c(cccc7c5)Oc5ccccc5-6)c4)cc3)cc2)cc1 ; c1ccc(-c2ccc(-c3ccc(N(c4ccc(-c5cccc6sc7ccccc7c56)cc4)c4ccccc4-c4cc5c6c(cccc6c4)Oc4ccccc4-5)cc3)cc2)cc1 ; c1ccc(-c2ccc(-c3ccc(N(c4ccc(-c5cccc6sc7ccccc7c56)cc4)c4ccccc4-c4ccc5c6c(cccc46)-c4ccccc4O5)cc3)cc2)cc1]

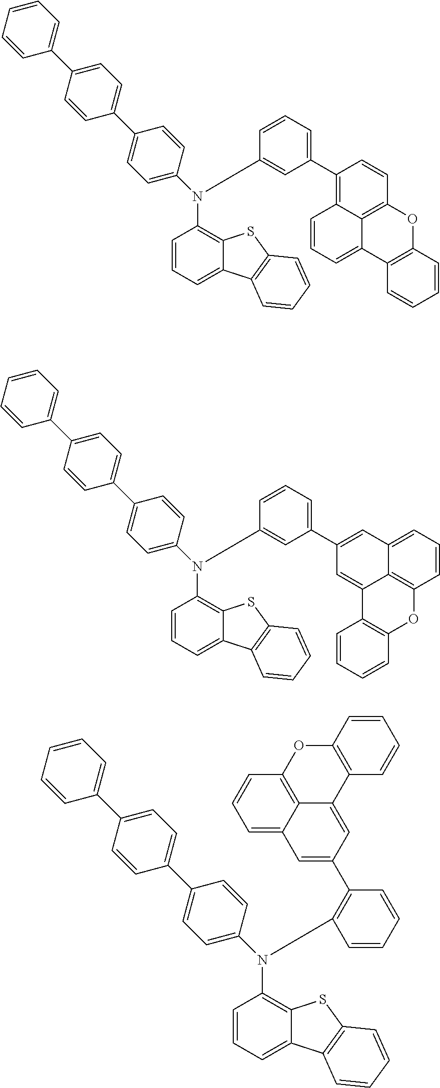

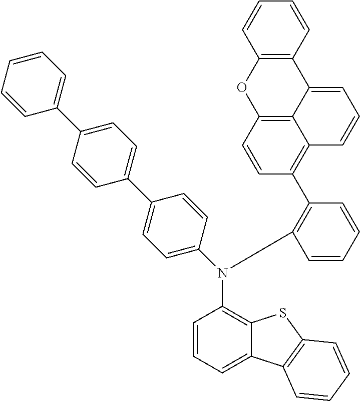 [SMILES: c1ccc(-c2ccc(-c3ccc(N(c4ccccc4-c4ccc5c6c(cccc46)-c4ccccc4O5)c4cccc5c4sc4ccccc45)cc3)cc2)cc1]

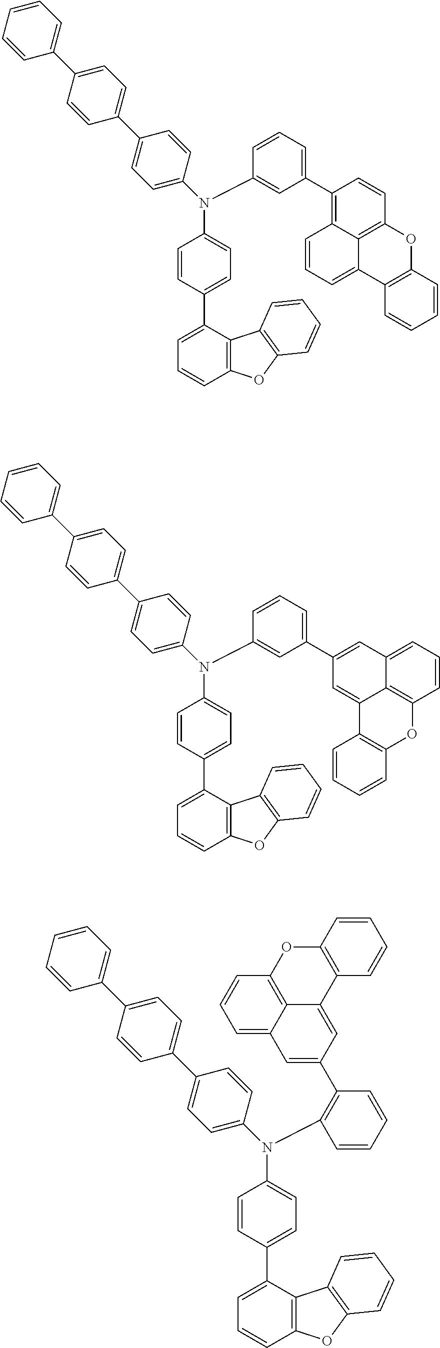 [SMILES: c1ccc(-c2ccc(-c3ccc(N(c4ccc(-c5cccc6oc7ccccc7c56)cc4)c4cccc(-c5ccc6c7c(cccc57)-c5ccccc5O6)c4)cc3)cc2)cc1 ; c1ccc(-c2ccc(-c3ccc(N(c4ccc(-c5cccc6oc7ccccc7c56)cc4)c4cccc(-c5cc6c7c(cccc7c5)Oc5ccccc5-6)c4)cc3)cc2)cc1 ; c1ccc(-c2ccc(-c3ccc(N(c4ccc(-c5cccc6oc7ccccc7c56)cc4)c4ccccc4-c4cc5c6c(cccc6c4)Oc4ccccc4-5)cc3)cc2)cc1]

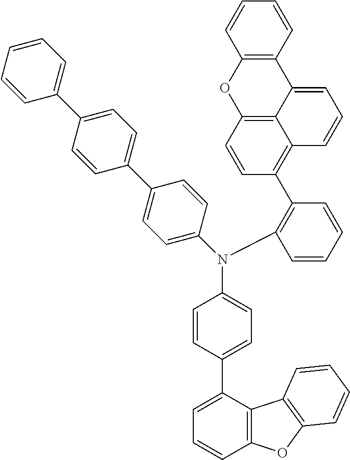 [SMILES: c1ccc(-c2ccc(-c3ccc(N(c4ccc(-c5cccc6oc7ccccc7c56)cc4)c4ccccc4-c4ccc5c6c(cccc46)-c4ccccc4O5)cc3)cc2)cc1]

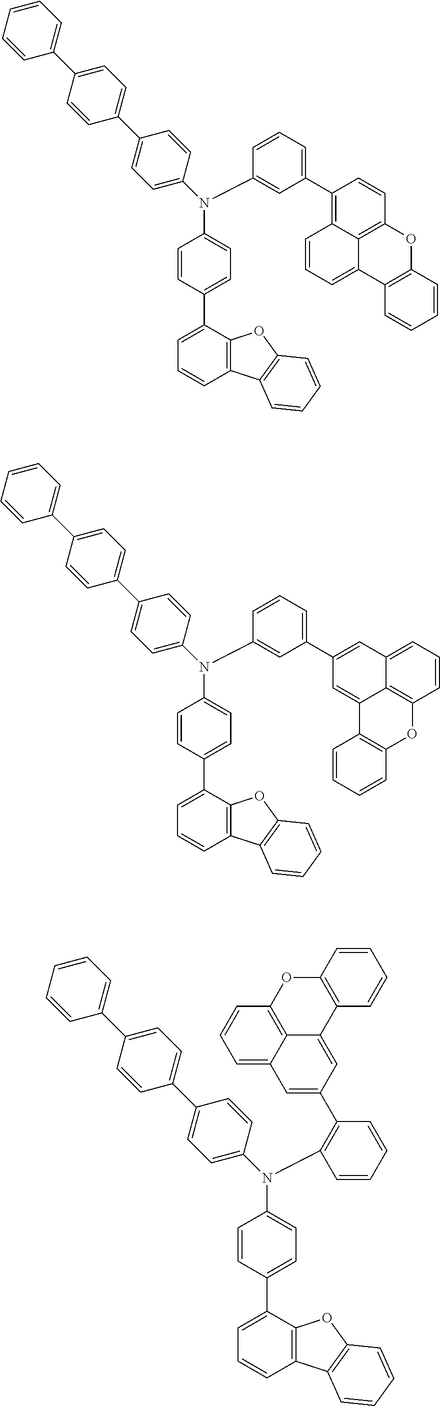 [SMILES: c1ccc(-c2ccc(-c3ccc(N(c4ccc(-c5cccc6c5oc5ccccc56)cc4)c4cccc(-c5ccc6c7c(cccc57)-c5ccccc5O6)c4)cc3)cc2)cc1 ; c1ccc(-c2ccc(-c3ccc(N(c4ccc(-c5cccc6c5oc5ccccc56)cc4)c4cccc(-c5cc6c7c(cccc7c5)Oc5ccccc5-6)c4)cc3)cc2)cc1 ; c1ccc(-c2ccc(-c3ccc(N(c4ccc(-c5cccc6c5oc5ccccc56)cc4)c4ccccc4-c4cc5c6c(cccc6c4)Oc4ccccc4-5)cc3)cc2)cc1]

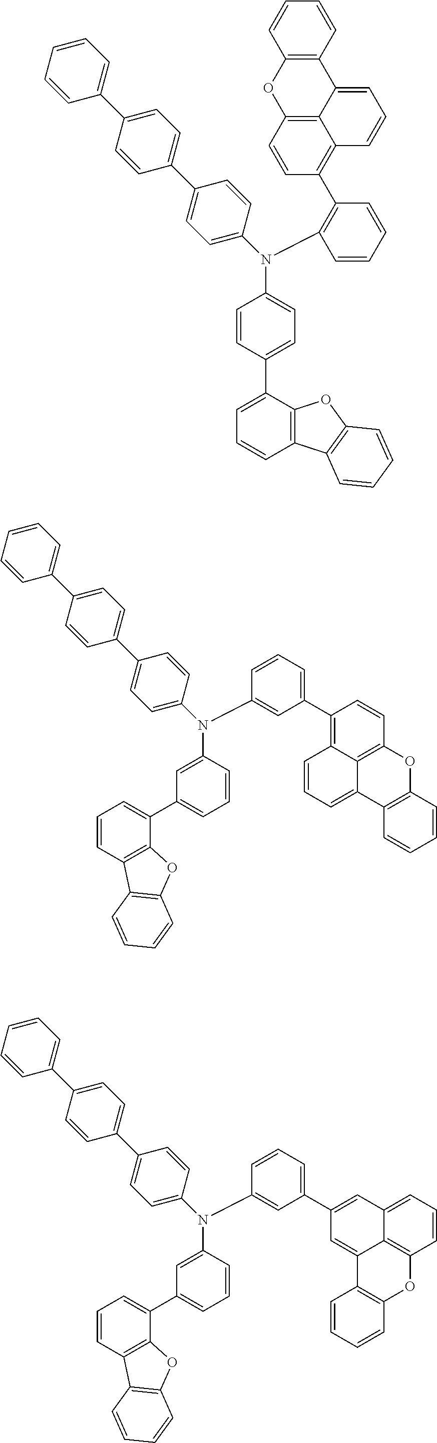 [SMILES: c1ccc(-c2ccc(-c3ccc(N(c4ccc(-c5cccc6c5oc5ccccc56)cc4)c4ccccc4-c4ccc5c6c(cccc46)-c4ccccc4O5)cc3)cc2)cc1 ; c1ccc(-c2ccc(-c3ccc(N(c4cccc(-c5ccc6c7c(cccc57)-c5ccccc5O6)c4)c4cccc(-c5cccc6c5oc5ccccc56)c4)cc3)cc2)cc1 ; c1ccc(-c2ccc(-c3ccc(N(c4cccc(-c5cc6c7c(cccc7c5)Oc5ccccc5-6)c4)c4cccc(-c5cccc6c5oc5ccccc56)c4)cc3)cc2)cc1]

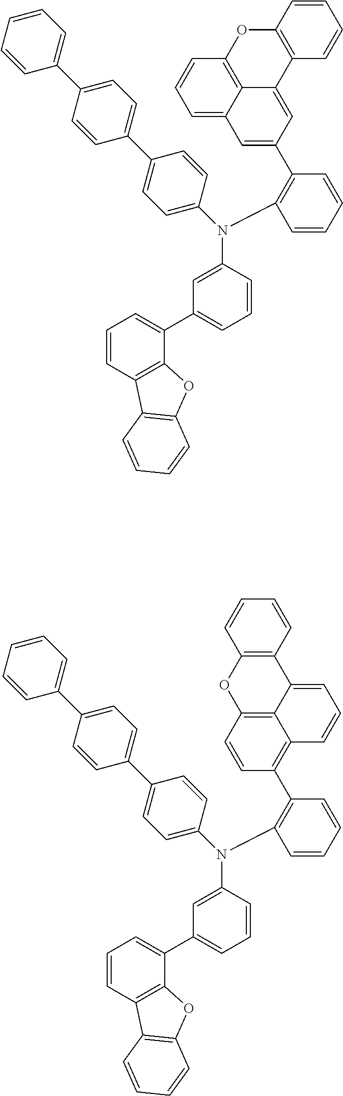 [SMILES: c1ccc(-c2ccc(-c3ccc(N(c4cccc(-c5cccc6c5oc5ccccc56)c4)c4ccccc4-c4cc5c6c(cccc6c4)Oc4ccccc4-5)cc3)cc2)cc1 ; c1ccc(-c2ccc(-c3ccc(N(c4cccc(-c5cccc6c5oc5ccccc56)c4)c4ccccc4-c4ccc5c6c(cccc46)-c4ccccc4O5)cc3)cc2)cc1]

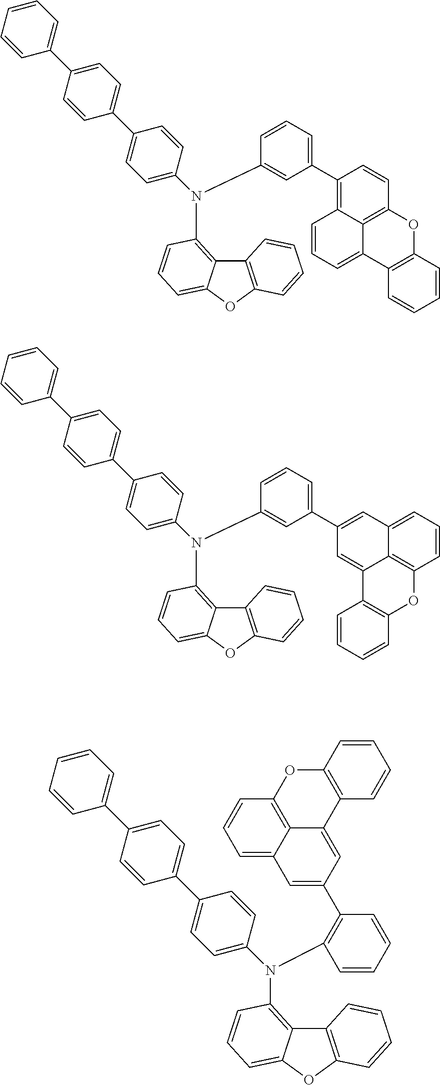 [SMILES: c1ccc(-c2ccc(-c3ccc(N(c4cccc(-c5ccc6c7c(cccc57)-c5ccccc5O6)c4)c4cccc5oc6ccccc6c45)cc3)cc2)cc1 ; c1ccc(-c2ccc(-c3ccc(N(c4cccc(-c5cc6c7c(cccc7c5)Oc5ccccc5-6)c4)c4cccc5oc6ccccc6c45)cc3)cc2)cc1 ; c1ccc(-c2ccc(-c3ccc(N(c4ccccc4-c4cc5c6c(cccc6c4)Oc4ccccc4-5)c4cccc5oc6ccccc6c45)cc3)cc2)cc1]

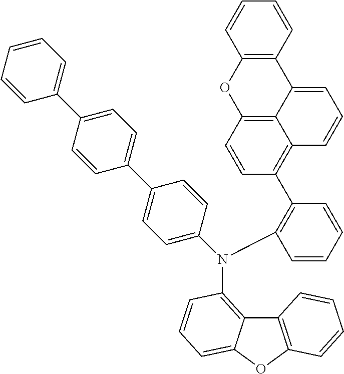 [SMILES: c1ccc(-c2ccc(-c3ccc(N(c4ccccc4-c4ccc5c6c(cccc46)-c4ccccc4O5)c4cccc5oc6ccccc6c45)cc3)cc2)cc1]

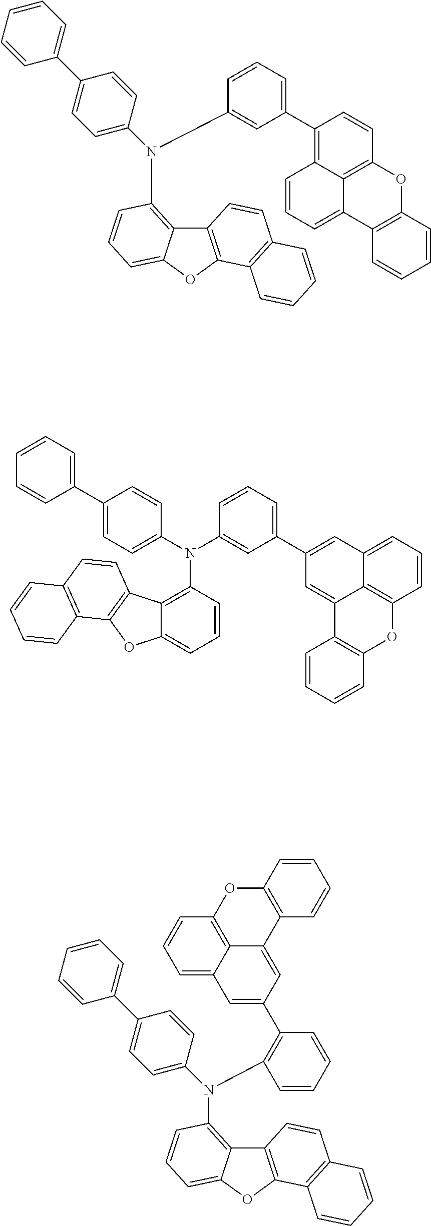 [SMILES: c1ccc(-c2ccc(N(c3cccc(-c4ccc5c6c(cccc46)-c4ccccc4O5)c3)c3cccc4oc5c6ccccc6ccc5c34)cc2)cc1 ; c1ccc(-c2ccc(N(c3cccc(-c4cc5c6c(cccc6c4)Oc4ccccc4-5)c3)c3cccc4oc5c6ccccc6ccc5c34)cc2)cc1 ; c1ccc(-c2ccc(N(c3ccccc3-c3cc4c5c(cccc5c3)Oc3ccccc3-4)c3cccc4oc5c6ccccc6ccc5c34)cc2)cc1]

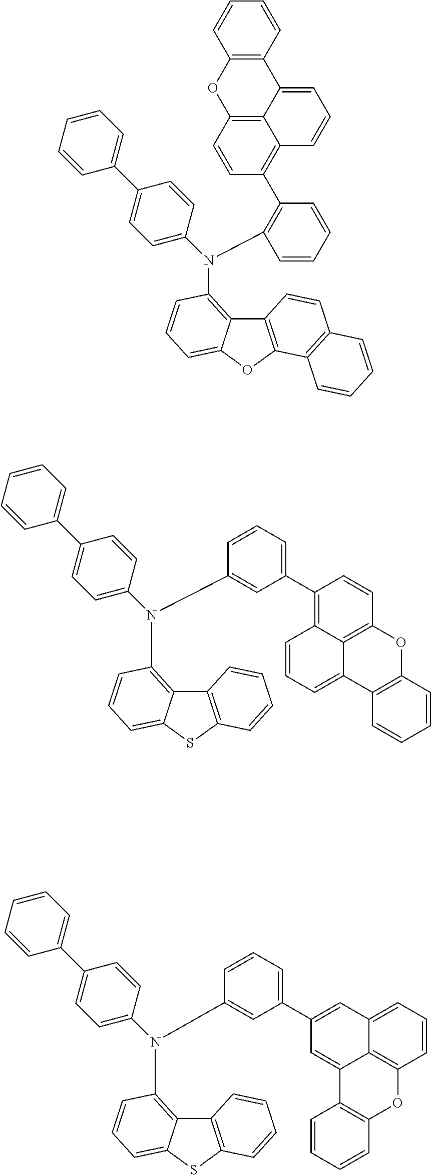 [SMILES: c1ccc(-c2ccc(N(c3ccccc3-c3ccc4c5c(cccc35)-c3ccccc3O4)c3cccc4oc5c6ccccc6ccc5c34)cc2)cc1 ; c1ccc(-c2ccc(N(c3cccc(-c4ccc5c6c(cccc46)-c4ccccc4O5)c3)c3cccc4sc5ccccc5c34)cc2)cc1 ; c1ccc(-c2ccc(N(c3cccc(-c4cc5c6c(cccc6c4)Oc4ccccc4-5)c3)c3cccc4sc5ccccc5c34)cc2)cc1]

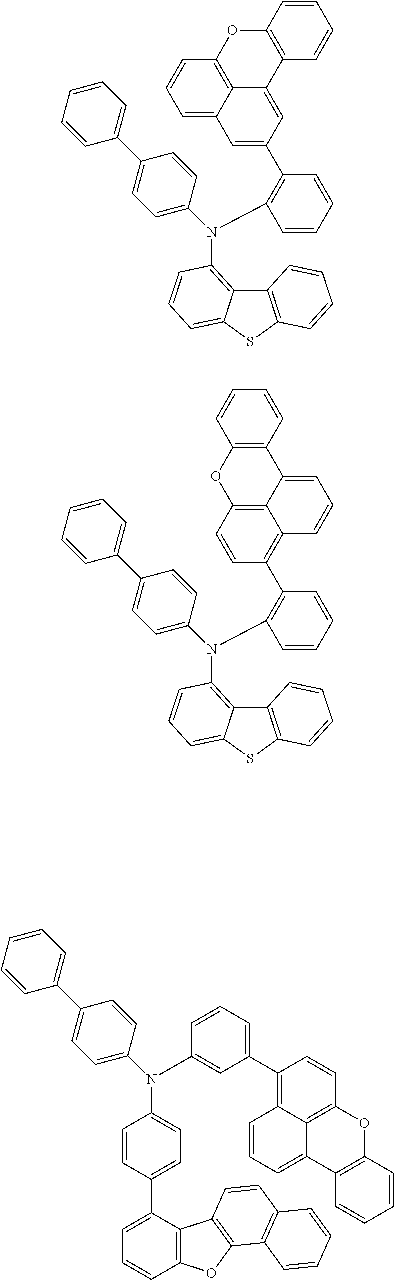 [SMILES: c1ccc(-c2ccc(N(c3ccccc3-c3cc4c5c(cccc5c3)Oc3ccccc3-4)c3cccc4sc5ccccc5c34)cc2)cc1 ; c1ccc(-c2ccc(N(c3ccccc3-c3ccc4c5c(cccc35)-c3ccccc3O4)c3cccc4sc5ccccc5c34)cc2)cc1 ; c1ccc(-c2ccc(N(c3ccc(-c4cccc5oc6c7ccccc7ccc6c45)cc3)c3cccc(-c4ccc5c6c(cccc46)-c4ccccc4O5)c3)cc2)cc1]

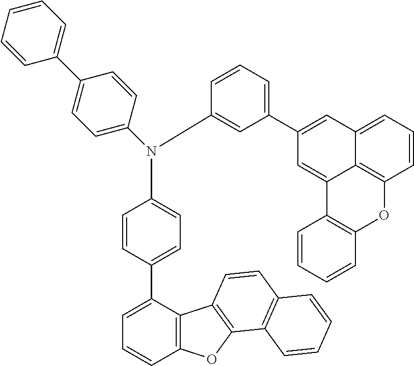 [SMILES: c1ccc(-c2ccc(N(c3ccc(-c4cccc5oc6c7ccccc7ccc6c45)cc3)c3cccc(-c4cc5c6c(cccc6c4)Oc4ccccc4-5)c3)cc2)cc1]

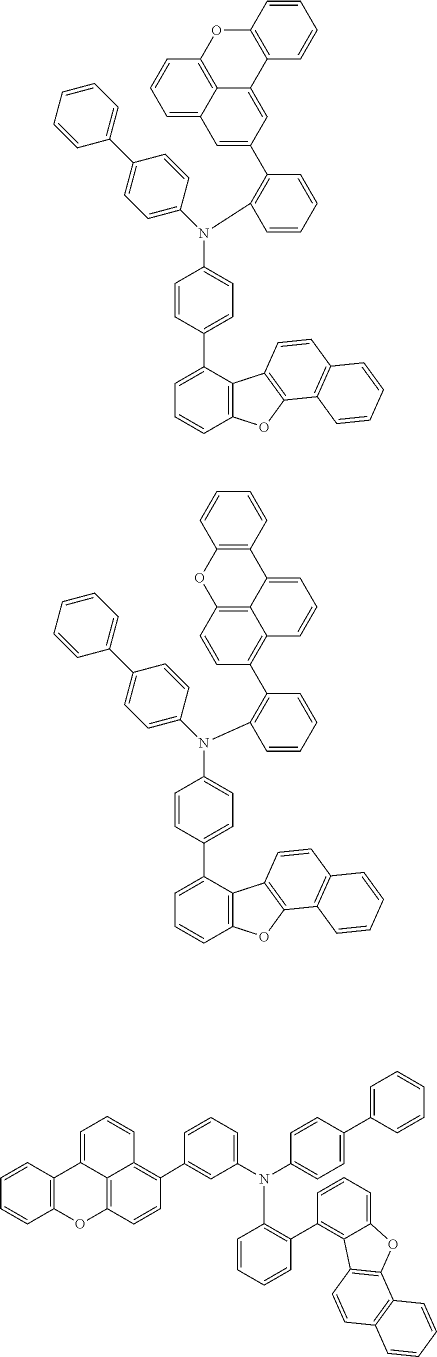 [SMILES: c1ccc(-c2ccc(N(c3ccc(-c4cccc5oc6c7ccccc7ccc6c45)cc3)c3ccccc3-c3cc4c5c(cccc5c3)Oc3ccccc3-4)cc2)cc1 ; c1ccc(-c2ccc(N(c3ccc(-c4cccc5oc6c7ccccc7ccc6c45)cc3)c3ccccc3-c3ccc4c5c(cccc35)-c3ccccc3O4)cc2)cc1 ; c1ccc(-c2ccc(N(c3cccc(-c4ccc5c6c(cccc46)-c4ccccc4O5)c3)c3ccccc3-c3cccc4oc5c6ccccc6ccc5c34)cc2)cc1]

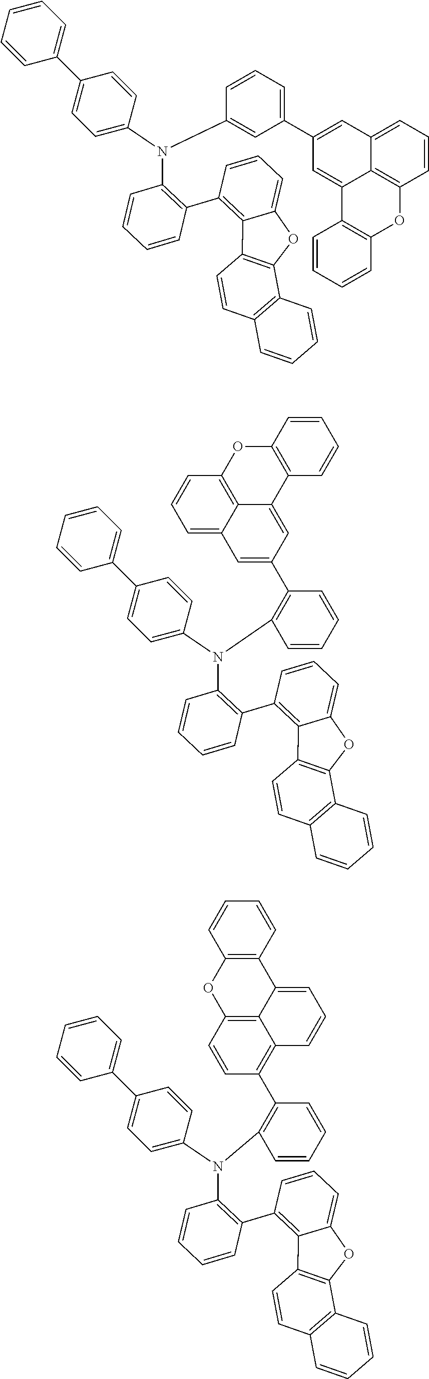 [SMILES: c1ccc(-c2ccc(N(c3cccc(-c4cc5c6c(cccc6c4)Oc4ccccc4-5)c3)c3ccccc3-c3cccc4oc5c6ccccc6ccc5c34)cc2)cc1 ; c1ccc(-c2ccc(N(c3ccccc3-c3cc4c5c(cccc5c3)Oc3ccccc3-4)c3ccccc3-c3cccc4oc5c6ccccc6ccc5c34)cc2)cc1 ; c1ccc(-c2ccc(N(c3ccccc3-c3ccc4c5c(cccc35)-c3ccccc3O4)c3ccccc3-c3cccc4oc5c6ccccc6ccc5c34)cc2)cc1]

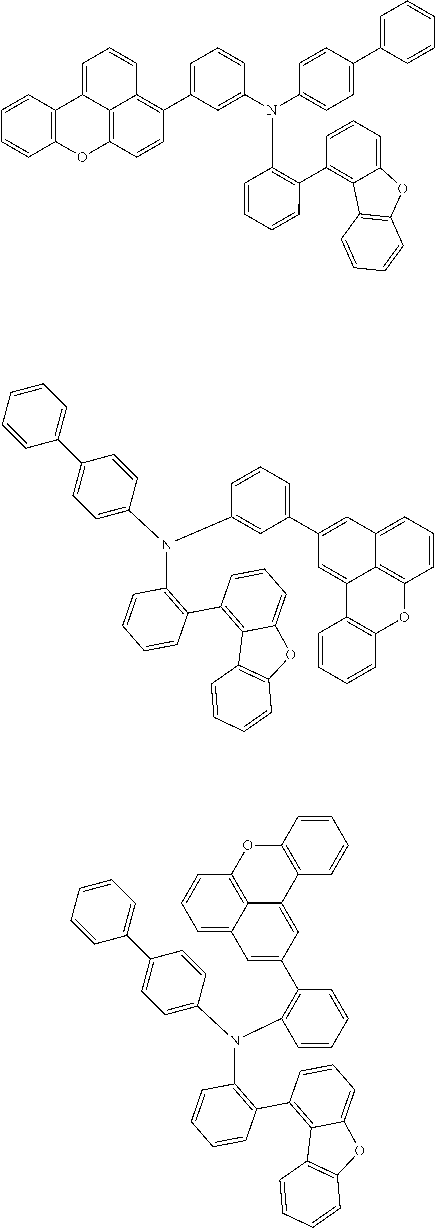 [SMILES: c1ccc(-c2ccc(N(c3cccc(-c4ccc5c6c(cccc46)-c4ccccc4O5)c3)c3ccccc3-c3cccc4oc5ccccc5c34)cc2)cc1 ; c1ccc(-c2ccc(N(c3cccc(-c4cc5c6c(cccc6c4)Oc4ccccc4-5)c3)c3ccccc3-c3cccc4oc5ccccc5c34)cc2)cc1 ; c1ccc(-c2ccc(N(c3ccccc3-c3cc4c5c(cccc5c3)Oc3ccccc3-4)c3ccccc3-c3cccc4oc5ccccc5c34)cc2)cc1]

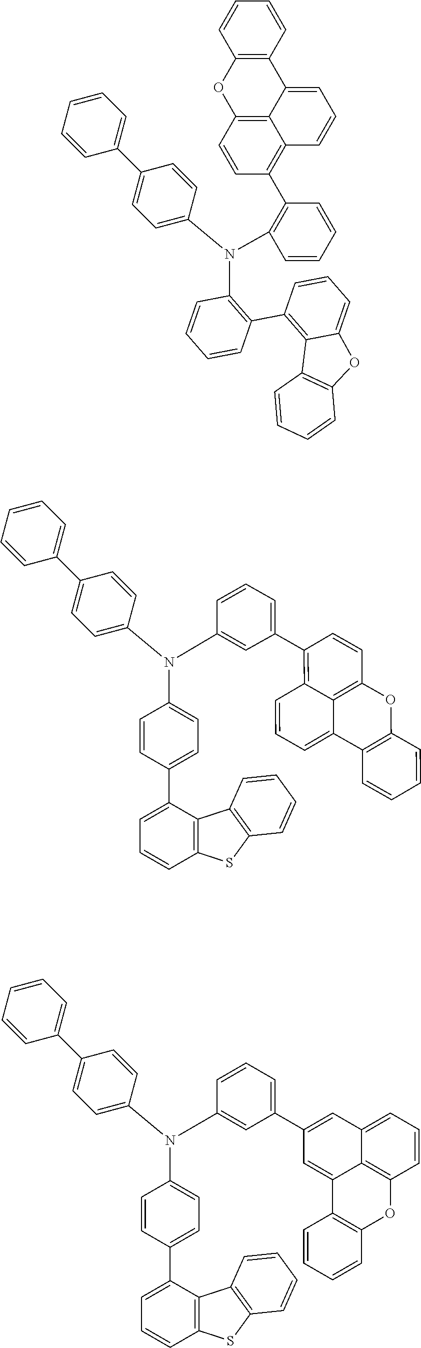 [SMILES: c1ccc(-c2ccc(N(c3ccccc3-c3ccc4c5c(cccc35)-c3ccccc3O4)c3ccccc3-c3cccc4oc5ccccc5c34)cc2)cc1 ; c1ccc(-c2ccc(N(c3ccc(-c4cccc5sc6ccccc6c45)cc3)c3cccc(-c4ccc5c6c(cccc46)-c4ccccc4O5)c3)cc2)cc1 ; c1ccc(-c2ccc(N(c3ccc(-c4cccc5sc6ccccc6c45)cc3)c3cccc(-c4cc5c6c(cccc6c4)Oc4ccccc4-5)c3)cc2)cc1]

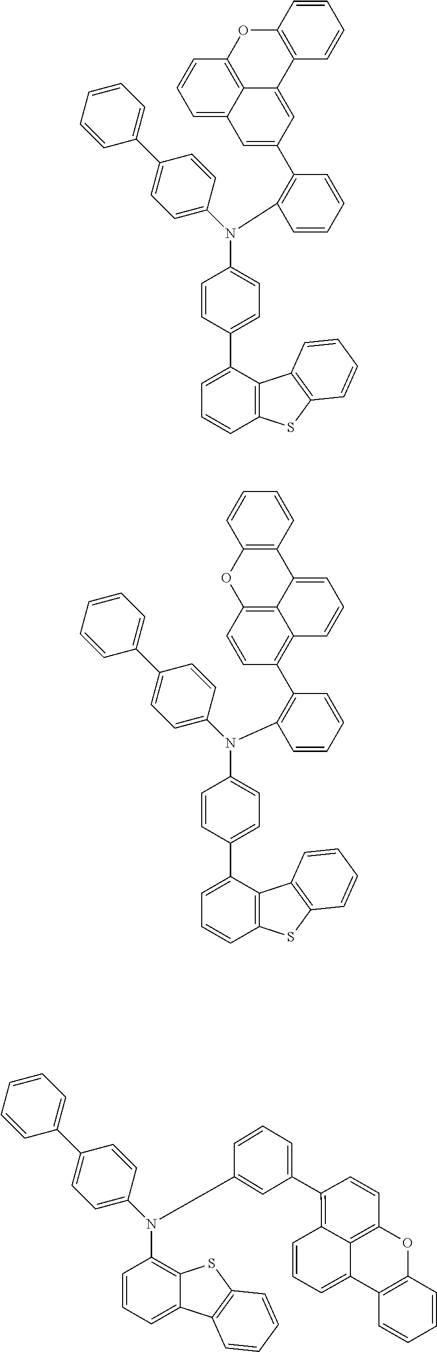 [SMILES: c1ccc(-c2ccc(N(c3ccc(-c4cccc5sc6ccccc6c45)cc3)c3ccccc3-c3cc4c5c(cccc5c3)Oc3ccccc3-4)cc2)cc1 ; c1ccc(-c2ccc(N(c3ccc(-c4cccc5sc6ccccc6c45)cc3)c3ccccc3-c3ccc4c5c(cccc35)-c3ccccc3O4)cc2)cc1 ; c1ccc(-c2ccc(N(c3cccc(-c4ccc5c6c(cccc46)-c4ccccc4O5)c3)c3cccc4c3sc3ccccc34)cc2)cc1]

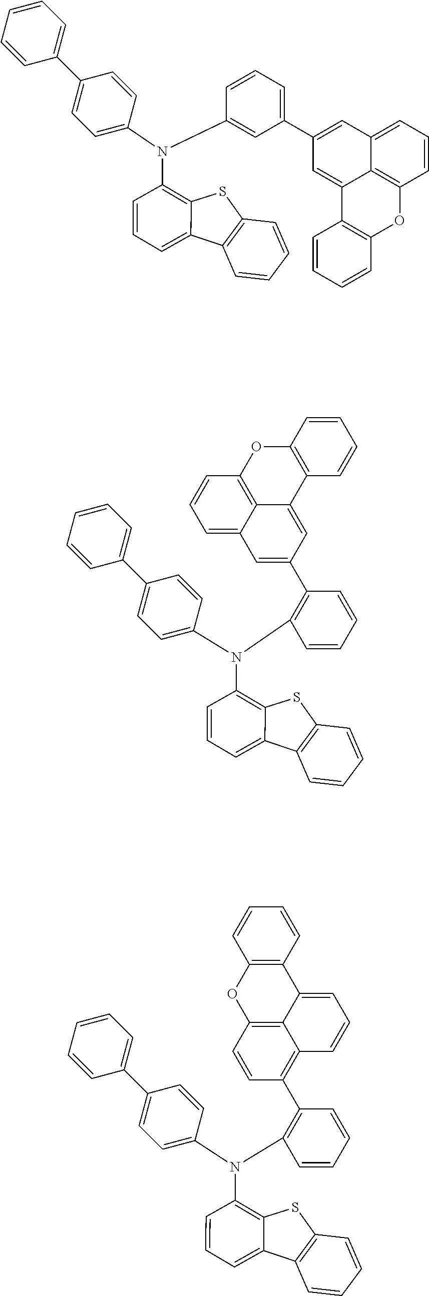 [SMILES: c1ccc(-c2ccc(N(c3cccc(-c4cc5c6c(cccc6c4)Oc4ccccc4-5)c3)c3cccc4c3sc3ccccc34)cc2)cc1 ; c1ccc(-c2ccc(N(c3ccccc3-c3cc4c5c(cccc5c3)Oc3ccccc3-4)c3cccc4c3sc3ccccc34)cc2)cc1 ; c1ccc(-c2ccc(N(c3ccccc3-c3ccc4c5c(cccc35)-c3ccccc3O4)c3cccc4c3sc3ccccc34)cc2)cc1]

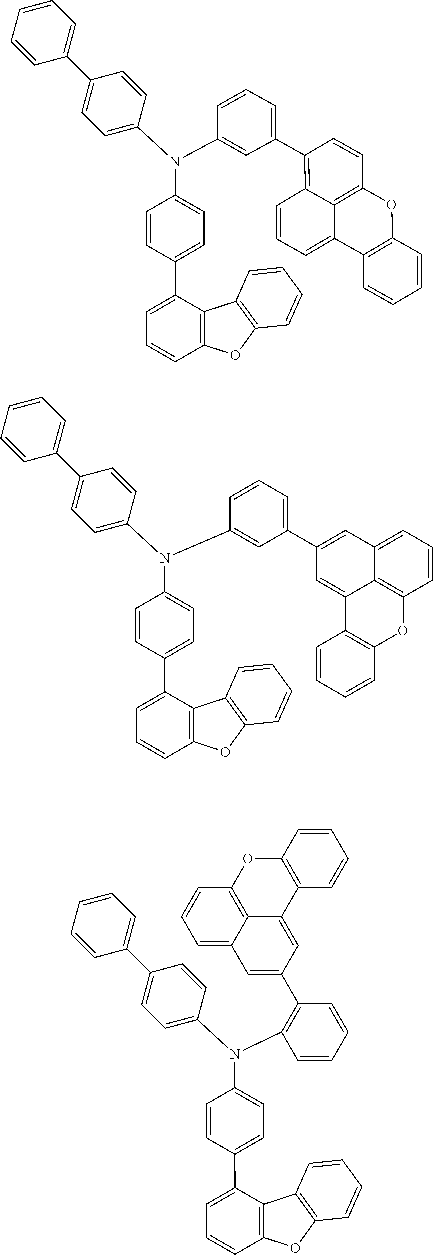 [SMILES: c1ccc(-c2ccc(N(c3ccc(-c4cccc5oc6ccccc6c45)cc3)c3cccc(-c4ccc5c6c(cccc46)-c4ccccc4O5)c3)cc2)cc1 ; c1ccc(-c2ccc(N(c3ccc(-c4cccc5oc6ccccc6c45)cc3)c3cccc(-c4cc5c6c(cccc6c4)Oc4ccccc4-5)c3)cc2)cc1 ; c1ccc(-c2ccc(N(c3ccc(-c4cccc5oc6ccccc6c45)cc3)c3ccccc3-c3cc4c5c(cccc5c3)Oc3ccccc3-4)cc2)cc1]

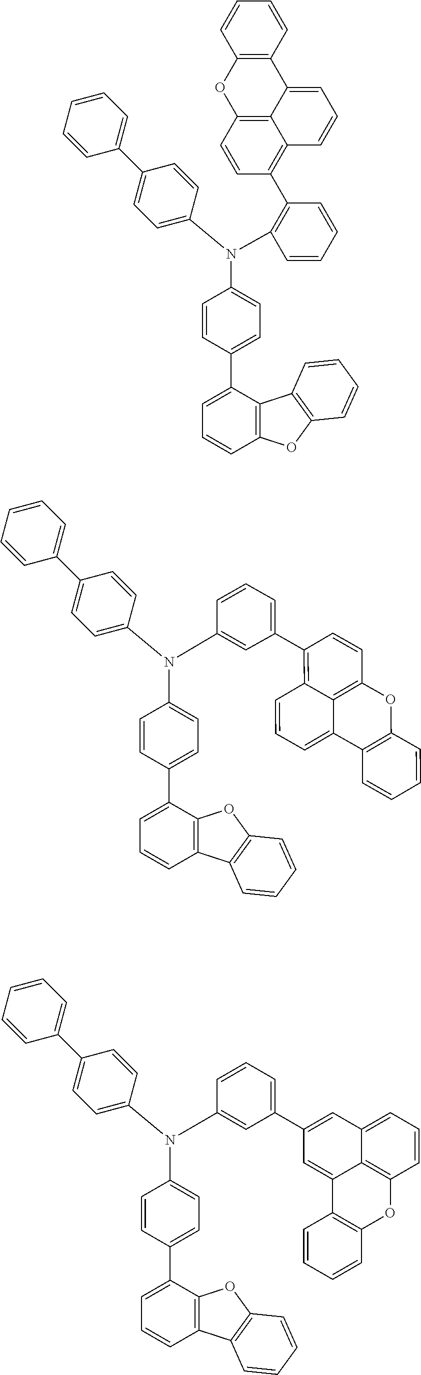 [SMILES: c1ccc(-c2ccc(N(c3ccc(-c4cccc5oc6ccccc6c45)cc3)c3ccccc3-c3ccc4c5c(cccc35)-c3ccccc3O4)cc2)cc1 ; c1ccc(-c2ccc(N(c3ccc(-c4cccc5c4oc4ccccc45)cc3)c3cccc(-c4ccc5c6c(cccc46)-c4ccccc4O5)c3)cc2)cc1 ; c1ccc(-c2ccc(N(c3ccc(-c4cccc5c4oc4ccccc45)cc3)c3cccc(-c4cc5c6c(cccc6c4)Oc4ccccc4-5)c3)cc2)cc1]

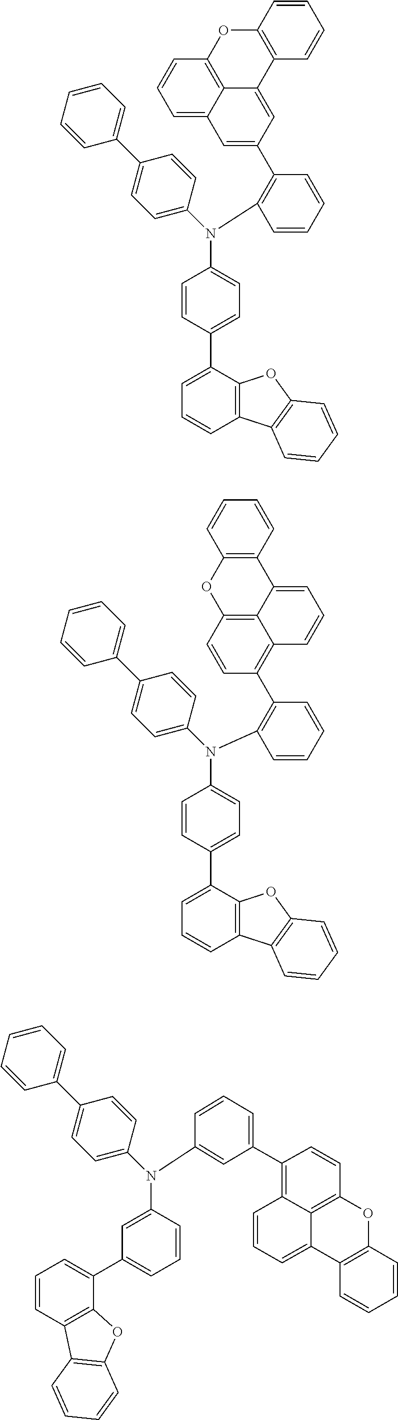 [SMILES: c1ccc(-c2ccc(N(c3ccc(-c4cccc5c4oc4ccccc45)cc3)c3ccccc3-c3cc4c5c(cccc5c3)Oc3ccccc3-4)cc2)cc1 ; c1ccc(-c2ccc(N(c3ccc(-c4cccc5c4oc4ccccc45)cc3)c3ccccc3-c3ccc4c5c(cccc35)-c3ccccc3O4)cc2)cc1 ; c1ccc(-c2ccc(N(c3cccc(-c4ccc5c6c(cccc46)-c4ccccc4O5)c3)c3cccc(-c4cccc5c4oc4ccccc45)c3)cc2)cc1]

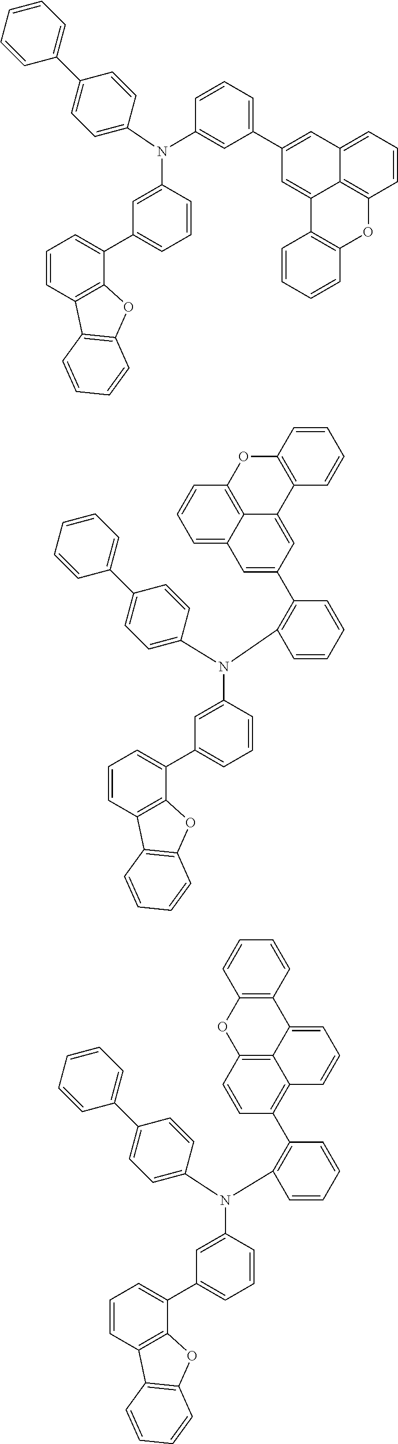 [SMILES: c1ccc(-c2ccc(N(c3cccc(-c4cc5c6c(cccc6c4)Oc4ccccc4-5)c3)c3cccc(-c4cccc5c4oc4ccccc45)c3)cc2)cc1 ; c1ccc(-c2ccc(N(c3cccc(-c4cccc5c4oc4ccccc45)c3)c3ccccc3-c3cc4c5c(cccc5c3)Oc3ccccc3-4)cc2)cc1 ; c1ccc(-c2ccc(N(c3cccc(-c4cccc5c4oc4ccccc45)c3)c3ccccc3-c3ccc4c5c(cccc35)-c3ccccc3O4)cc2)cc1]

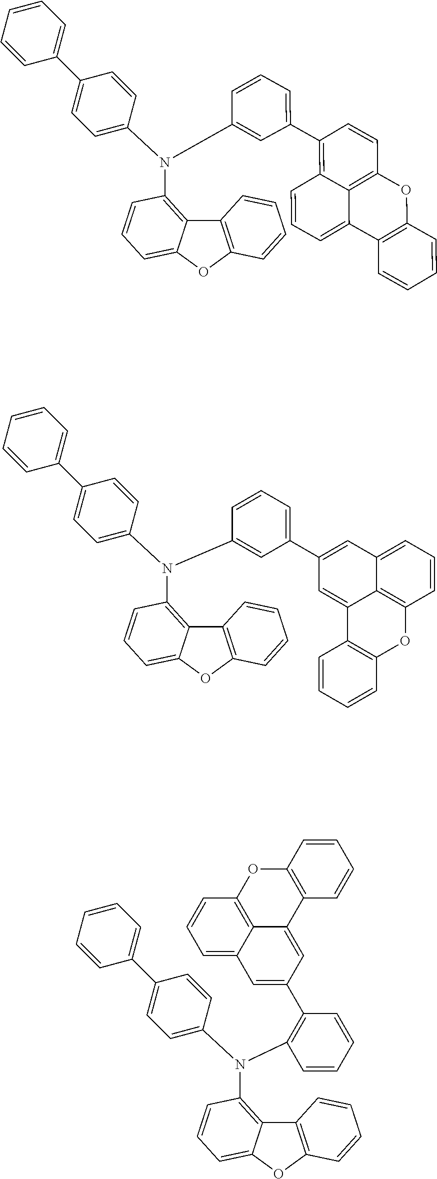 [SMILES: c1ccc(-c2ccc(N(c3cccc(-c4ccc5c6c(cccc46)-c4ccccc4O5)c3)c3cccc4oc5ccccc5c34)cc2)cc1 ; c1ccc(-c2ccc(N(c3cccc(-c4cc5c6c(cccc6c4)Oc4ccccc4-5)c3)c3cccc4oc5ccccc5c34)cc2)cc1 ; c1ccc(-c2ccc(N(c3ccccc3-c3cc4c5c(cccc5c3)Oc3ccccc3-4)c3cccc4oc5ccccc5c34)cc2)cc1]

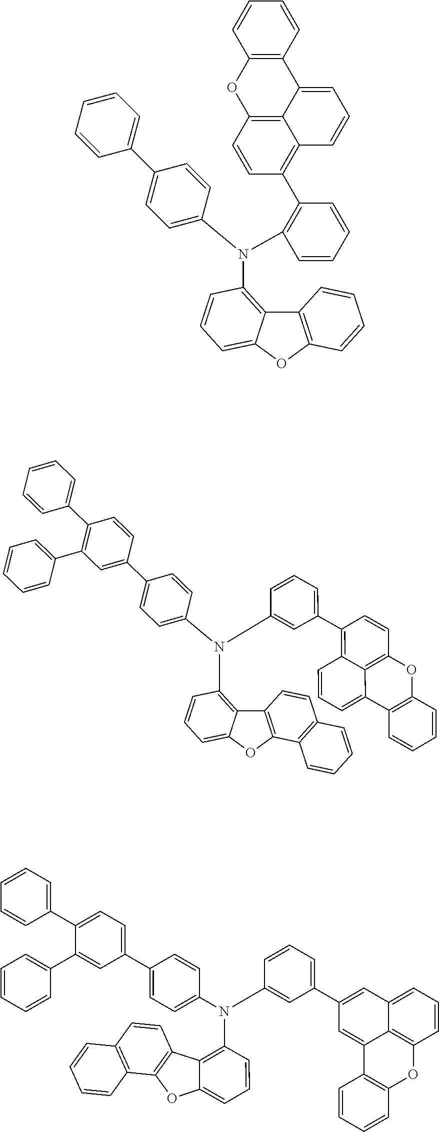 [SMILES: c1ccc(-c2ccc(N(c3ccccc3-c3ccc4c5c(cccc35)-c3ccccc3O4)c3cccc4oc5ccccc5c34)cc2)cc1 ; c1ccc(-c2ccc(-c3ccc(N(c4cccc(-c5ccc6c7c(cccc57)-c5ccccc5O6)c4)c4cccc5oc6c7ccccc7ccc6c45)cc3)cc2-c2ccccc2)cc1 ; c1ccc(-c2ccc(-c3ccc(N(c4cccc(-c5cc6c7c(cccc7c5)Oc5ccccc5-6)c4)c4cccc5oc6c7ccccc7ccc6c45)cc3)cc2-c2ccccc2)cc1]

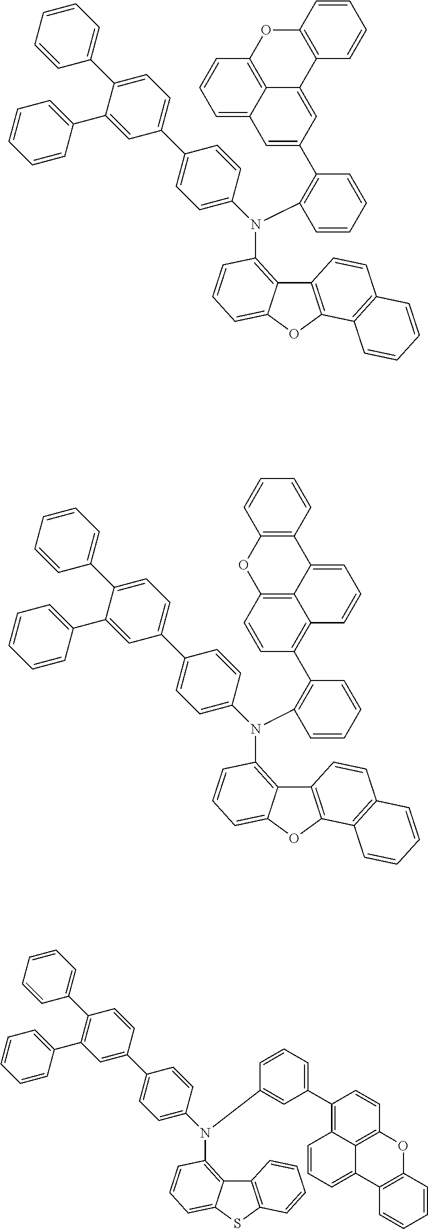 [SMILES: c1ccc(-c2ccc(-c3ccc(N(c4ccccc4-c4cc5c6c(cccc6c4)Oc4ccccc4-5)c4cccc5oc6c7ccccc7ccc6c45)cc3)cc2-c2ccccc2)cc1 ; c1ccc(-c2ccc(-c3ccc(N(c4ccccc4-c4ccc5c6c(cccc46)-c4ccccc4O5)c4cccc5oc6c7ccccc7ccc6c45)cc3)cc2-c2ccccc2)cc1 ; c1ccc(-c2ccc(-c3ccc(N(c4cccc(-c5ccc6c7c(cccc57)-c5ccccc5O6)c4)c4cccc5sc6ccccc6c45)cc3)cc2-c2ccccc2)cc1]

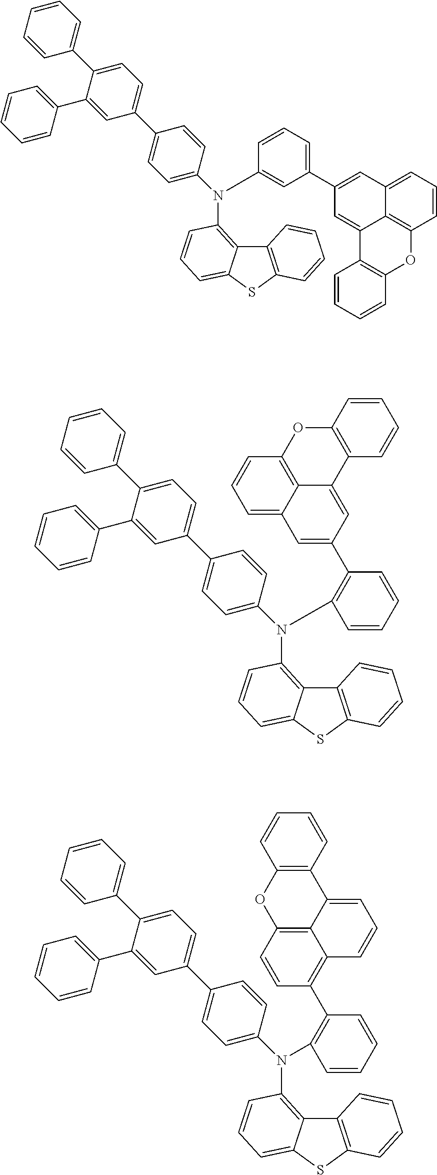 [SMILES: c1ccc(-c2ccc(-c3ccc(N(c4cccc(-c5cc6c7c(cccc7c5)Oc5ccccc5-6)c4)c4cccc5sc6ccccc6c45)cc3)cc2-c2ccccc2)cc1 ; c1ccc(-c2ccc(-c3ccc(N(c4ccccc4-c4cc5c6c(cccc6c4)Oc4ccccc4-5)c4cccc5sc6ccccc6c45)cc3)cc2-c2ccccc2)cc1 ; c1ccc(-c2ccc(-c3ccc(N(c4ccccc4-c4ccc5c6c(cccc46)-c4ccccc4O5)c4cccc5sc6ccccc6c45)cc3)cc2-c2ccccc2)cc1]

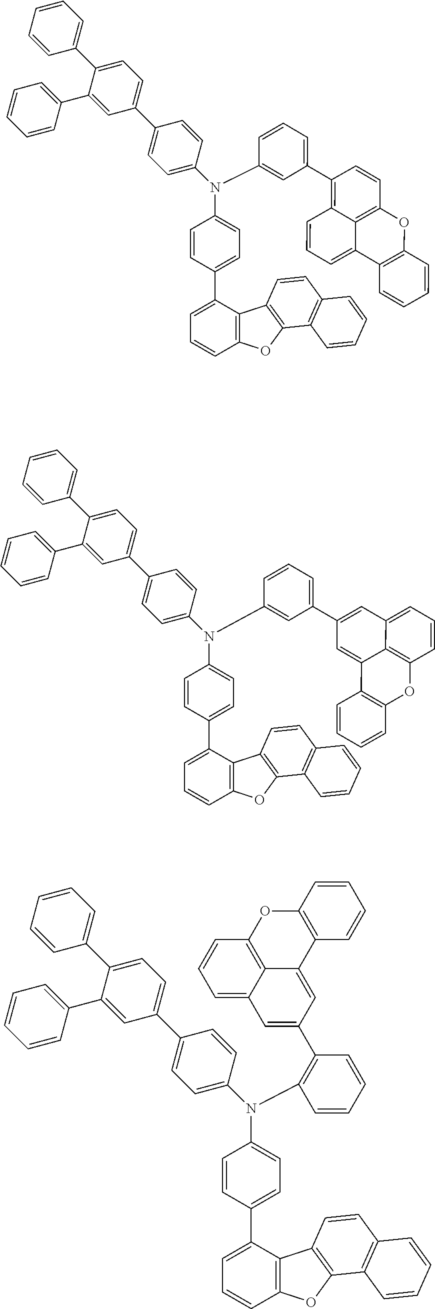 [SMILES: c1ccc(-c2ccc(-c3ccc(N(c4ccc(-c5cccc6oc7c8ccccc8ccc7c56)cc4)c4cccc(-c5ccc6c7c(cccc57)-c5ccccc5O6)c4)cc3)cc2-c2ccccc2)cc1 ; c1ccc(-c2ccc(-c3ccc(N(c4ccc(-c5cccc6oc7c8ccccc8ccc7c56)cc4)c4cccc(-c5cc6c7c(cccc7c5)Oc5ccccc5-6)c4)cc3)cc2-c2ccccc2)cc1 ; c1ccc(-c2ccc(-c3ccc(N(c4ccc(-c5cccc6oc7c8ccccc8ccc7c56)cc4)c4ccccc4-c4cc5c6c(cccc6c4)Oc4ccccc4-5)cc3)cc2-c2ccccc2)cc1]

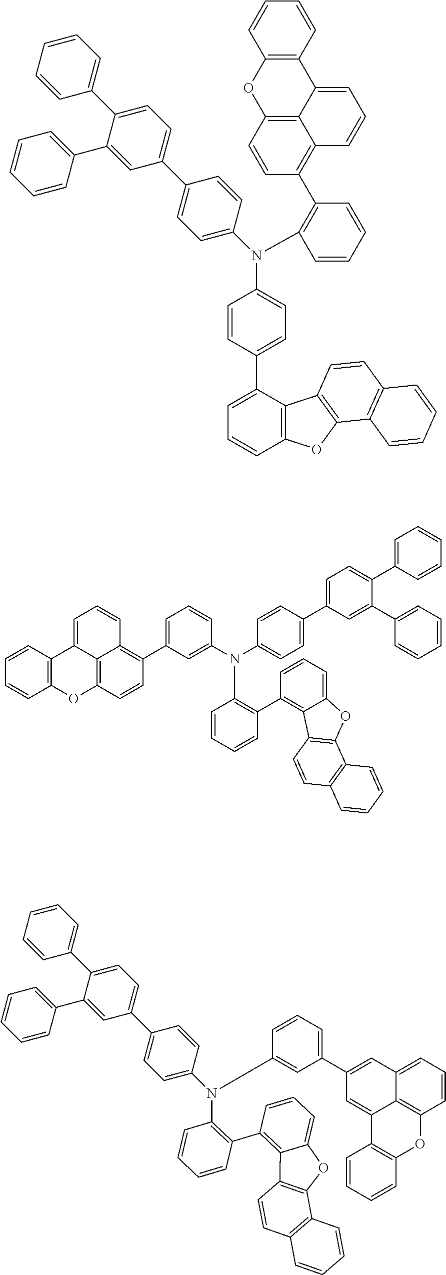 [SMILES: c1ccc(-c2ccc(-c3ccc(N(c4ccc(-c5cccc6oc7c8ccccc8ccc7c56)cc4)c4ccccc4-c4ccc5c6c(cccc46)-c4ccccc4O5)cc3)cc2-c2ccccc2)cc1 ; c1ccc(-c2ccc(-c3ccc(N(c4cccc(-c5ccc6c7c(cccc57)-c5ccccc5O6)c4)c4ccccc4-c4cccc5oc6c7ccccc7ccc6c45)cc3)cc2-c2ccccc2)cc1 ; c1ccc(-c2ccc(-c3ccc(N(c4cccc(-c5cc6c7c(cccc7c5)Oc5ccccc5-6)c4)c4ccccc4-c4cccc5oc6c7ccccc7ccc6c45)cc3)cc2-c2ccccc2)cc1]

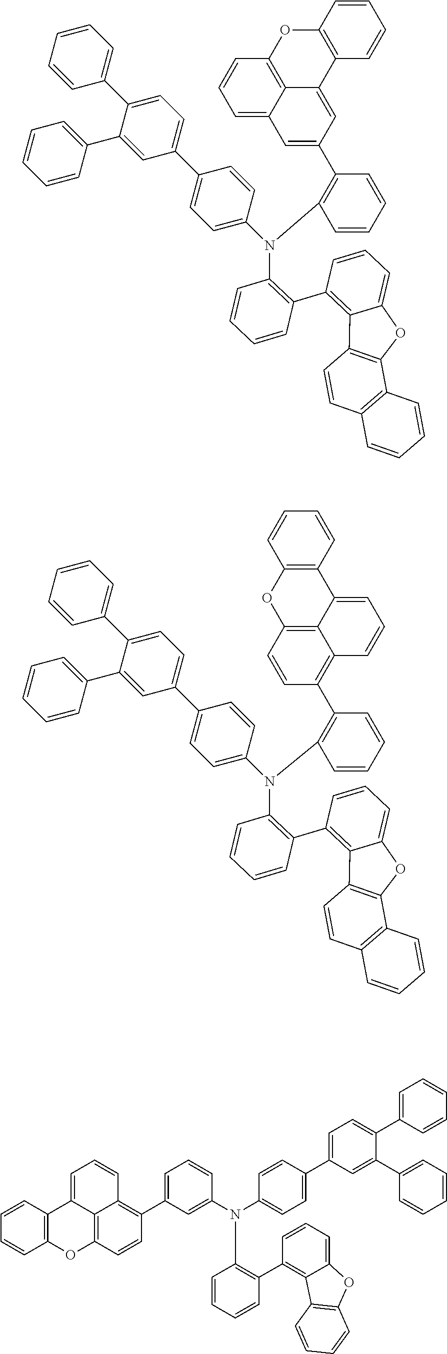 [SMILES: c1ccc(-c2ccc(-c3ccc(N(c4ccccc4-c4cc5c6c(cccc6c4)Oc4ccccc4-5)c4ccccc4-c4cccc5oc6c7ccccc7ccc6c45)cc3)cc2-c2ccccc2)cc1 ; c1ccc(-c2ccc(-c3ccc(N(c4ccccc4-c4ccc5c6c(cccc46)-c4ccccc4O5)c4ccccc4-c4cccc5oc6c7ccccc7ccc6c45)cc3)cc2-c2ccccc2)cc1 ; c1ccc(-c2ccc(-c3ccc(N(c4cccc(-c5ccc6c7c(cccc57)-c5ccccc5O6)c4)c4ccccc4-c4cccc5oc6ccccc6c45)cc3)cc2-c2ccccc2)cc1]

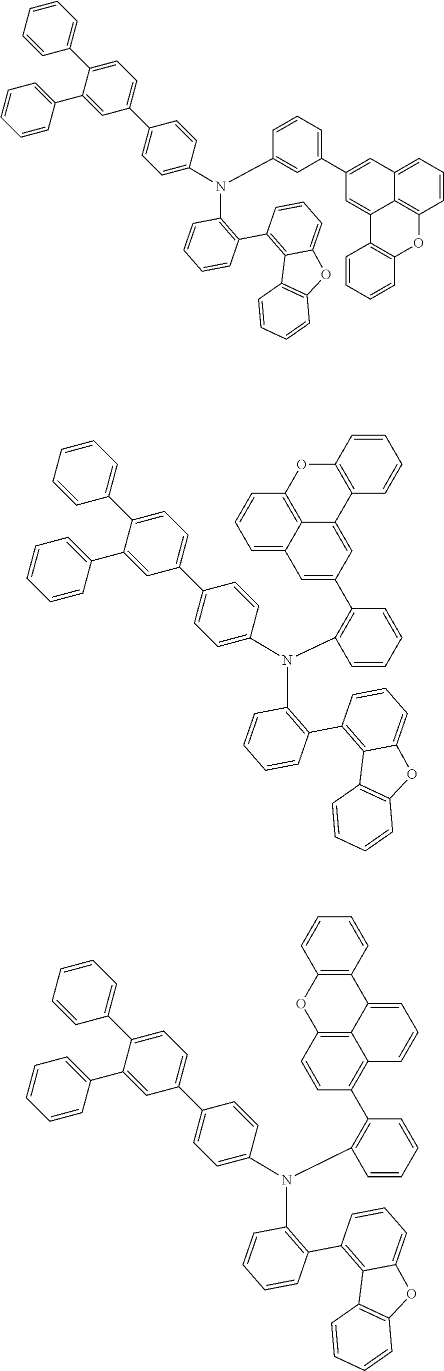 [SMILES: c1ccc(-c2ccc(-c3ccc(N(c4cccc(-c5cc6c7c(cccc7c5)Oc5ccccc5-6)c4)c4ccccc4-c4cccc5oc6ccccc6c45)cc3)cc2-c2ccccc2)cc1 ; c1ccc(-c2ccc(-c3ccc(N(c4ccccc4-c4cc5c6c(cccc6c4)Oc4ccccc4-5)c4ccccc4-c4cccc5oc6ccccc6c45)cc3)cc2-c2ccccc2)cc1 ; c1ccc(-c2ccc(-c3ccc(N(c4ccccc4-c4ccc5c6c(cccc46)-c4ccccc4O5)c4ccccc4-c4cccc5oc6ccccc6c45)cc3)cc2-c2ccccc2)cc1]

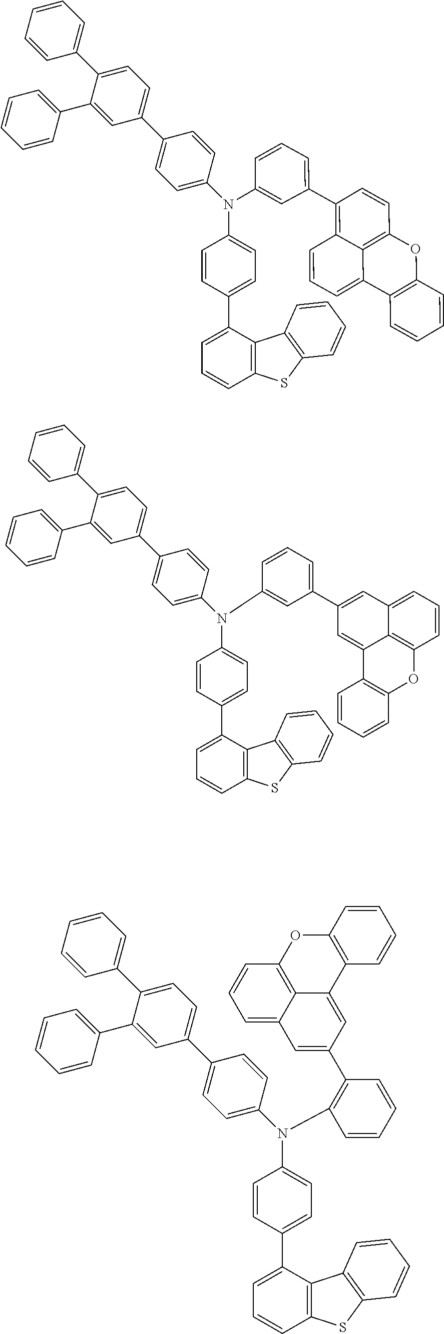 [SMILES: c1ccc(-c2ccc(-c3ccc(N(c4ccc(-c5cccc6sc7ccccc7c56)cc4)c4cccc(-c5ccc6c7c(cccc57)-c5ccccc5O6)c4)cc3)cc2-c2ccccc2)cc1 ; c1ccc(-c2ccc(-c3ccc(N(c4ccc(-c5cccc6sc7ccccc7c56)cc4)c4cccc(-c5cc6c7c(cccc7c5)Oc5ccccc5-6)c4)cc3)cc2-c2ccccc2)cc1 ; c1ccc(-c2ccc(-c3ccc(N(c4ccc(-c5cccc6sc7ccccc7c56)cc4)c4ccccc4-c4cc5c6c(cccc6c4)Oc4ccccc4-5)cc3)cc2-c2ccccc2)cc1]

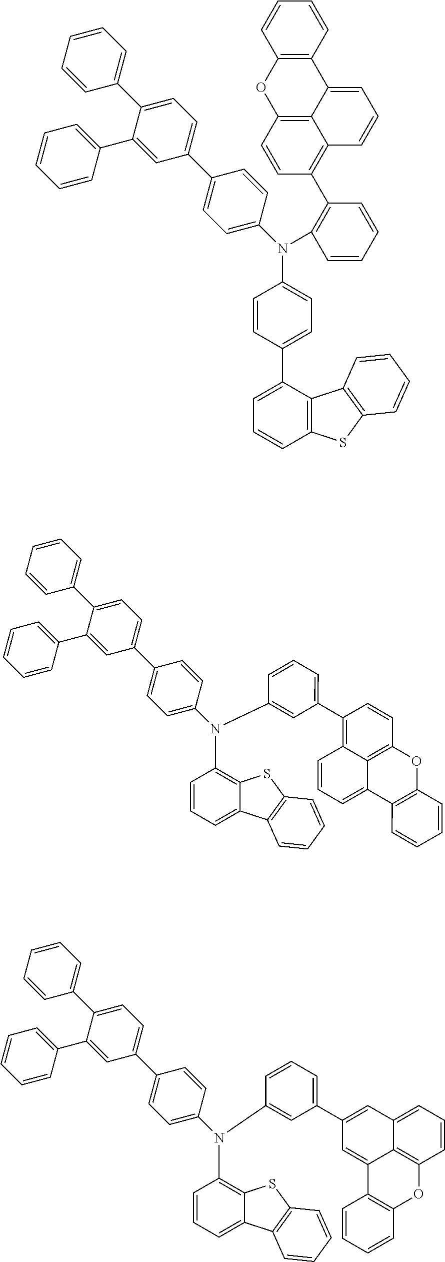 [SMILES: c1ccc(-c2ccc(-c3ccc(N(c4ccc(-c5cccc6sc7ccccc7c56)cc4)c4ccccc4-c4ccc5c6c(cccc46)-c4ccccc4O5)cc3)cc2-c2ccccc2)cc1 ; c1ccc(-c2ccc(-c3ccc(N(c4cccc(-c5ccc6c7c(cccc57)-c5ccccc5O6)c4)c4cccc5c4sc4ccccc45)cc3)cc2-c2ccccc2)cc1 ; c1ccc(-c2ccc(-c3ccc(N(c4cccc(-c5cc6c7c(cccc7c5)Oc5ccccc5-6)c4)c4cccc5c4sc4ccccc45)cc3)cc2-c2ccccc2)cc1]

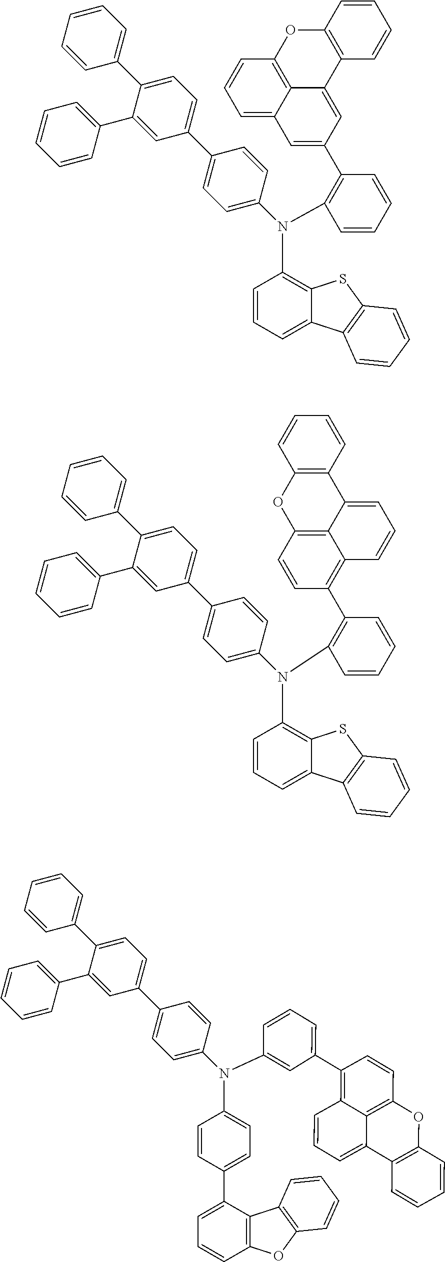 [SMILES: c1ccc(-c2ccc(-c3ccc(N(c4ccccc4-c4cc5c6c(cccc6c4)Oc4ccccc4-5)c4cccc5c4sc4ccccc45)cc3)cc2-c2ccccc2)cc1 ; c1ccc(-c2ccc(-c3ccc(N(c4ccccc4-c4ccc5c6c(cccc46)-c4ccccc4O5)c4cccc5c4sc4ccccc45)cc3)cc2-c2ccccc2)cc1 ; c1ccc(-c2ccc(-c3ccc(N(c4ccc(-c5cccc6oc7ccccc7c56)cc4)c4cccc(-c5ccc6c7c(cccc57)-c5ccccc5O6)c4)cc3)cc2-c2ccccc2)cc1]

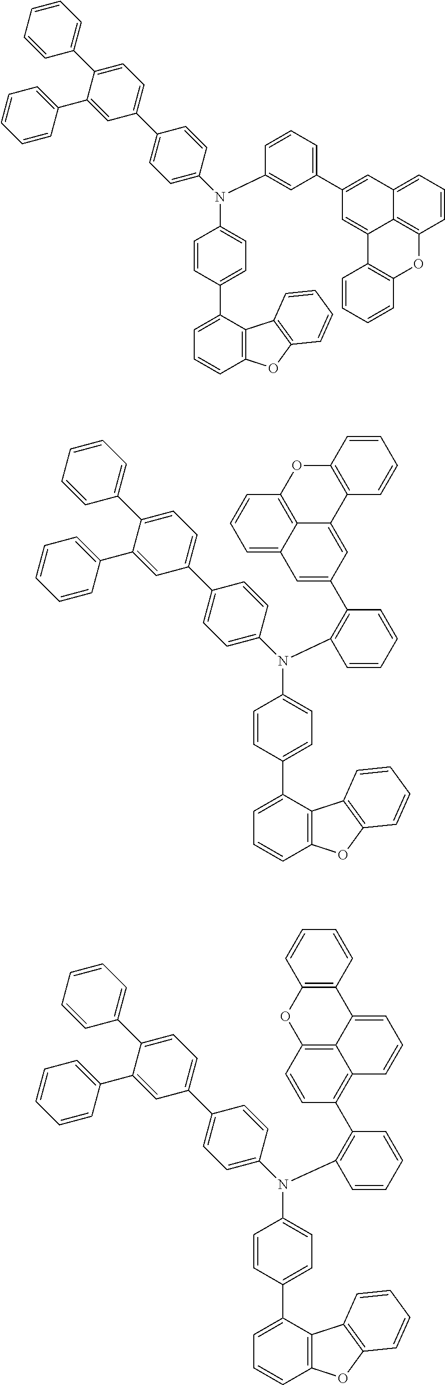 [SMILES: c1ccc(-c2ccc(-c3ccc(N(c4ccc(-c5cccc6oc7ccccc7c56)cc4)c4cccc(-c5cc6c7c(cccc7c5)Oc5ccccc5-6)c4)cc3)cc2-c2ccccc2)cc1 ; c1ccc(-c2ccc(-c3ccc(N(c4ccc(-c5cccc6oc7ccccc7c56)cc4)c4ccccc4-c4cc5c6c(cccc6c4)Oc4ccccc4-5)cc3)cc2-c2ccccc2)cc1 ; c1ccc(-c2ccc(-c3ccc(N(c4ccc(-c5cccc6oc7ccccc7c56)cc4)c4ccccc4-c4ccc5c6c(cccc46)-c4ccccc4O5)cc3)cc2-c2ccccc2)cc1]

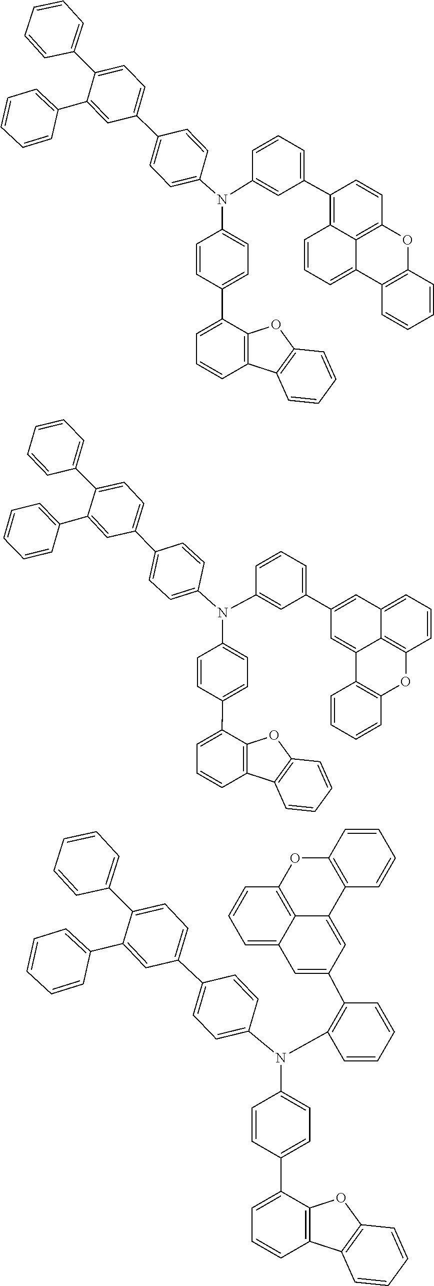

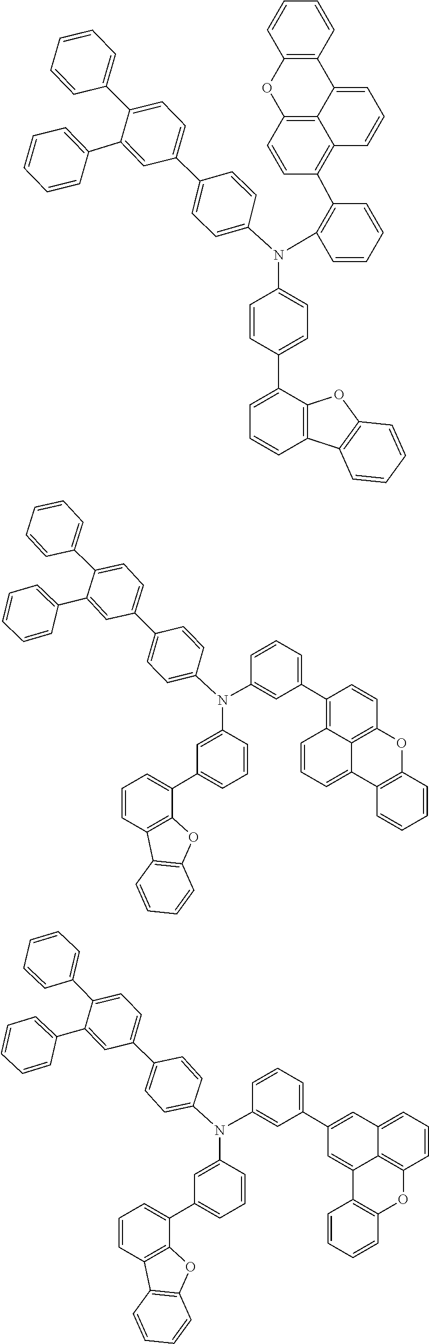 [SMILES: c1ccc(-c2ccc(-c3ccc(N(c4ccc(-c5cccc6c5oc5ccccc56)cc4)c4ccccc4-c4ccc5c6c(cccc46)-c4ccccc4O5)cc3)cc2-c2ccccc2)cc1 ; c1ccc(-c2ccc(-c3ccc(N(c4cccc(-c5ccc6c7c(cccc57)-c5ccccc5O6)c4)c4cccc(-c5cccc6c5oc5ccccc56)c4)cc3)cc2-c2ccccc2)cc1 ; c1ccc(-c2ccc(-c3ccc(N(c4cccc(-c5cc6c7c(cccc7c5)Oc5ccccc5-6)c4)c4cccc(-c5cccc6c5oc5ccccc56)c4)cc3)cc2-c2ccccc2)cc1]

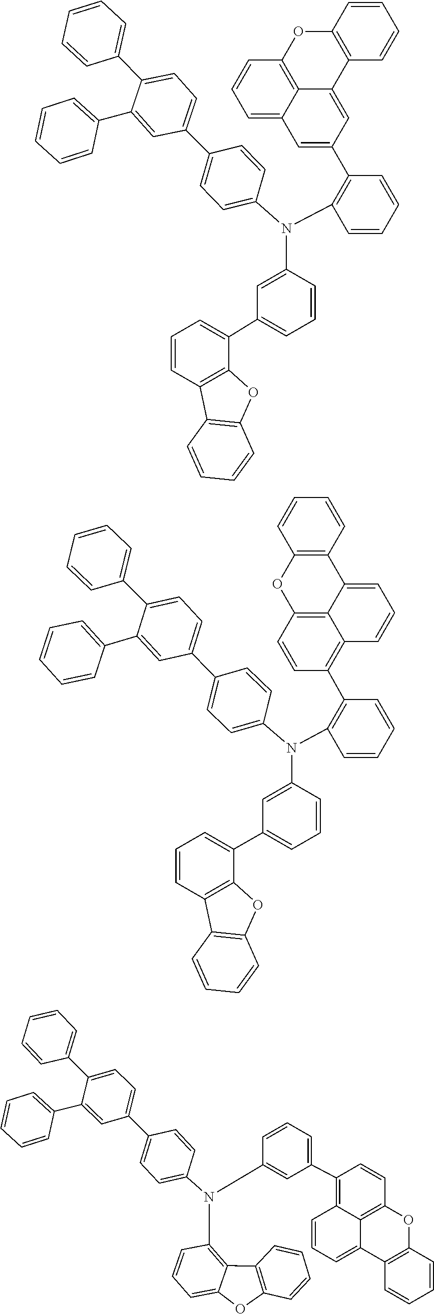 [SMILES: c1ccc(-c2ccc(-c3ccc(N(c4cccc(-c5cccc6c5oc5ccccc56)c4)c4ccccc4-c4cc5c6c(cccc6c4)Oc4ccccc4-5)cc3)cc2-c2ccccc2)cc1 ; c1ccc(-c2ccc(-c3ccc(N(c4cccc(-c5cccc6c5oc5ccccc56)c4)c4ccccc4-c4ccc5c6c(cccc46)-c4ccccc4O5)cc3)cc2-c2ccccc2)cc1 ; c1ccc(-c2ccc(-c3ccc(N(c4cccc(-c5ccc6c7c(cccc57)-c5ccccc5O6)c4)c4cccc5oc6ccccc6c45)cc3)cc2-c2ccccc2)cc1]

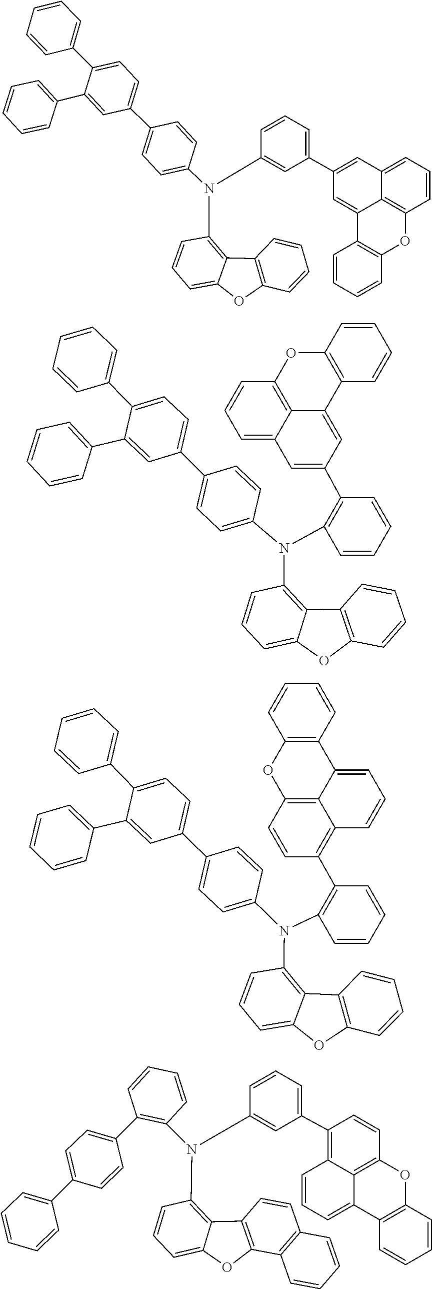

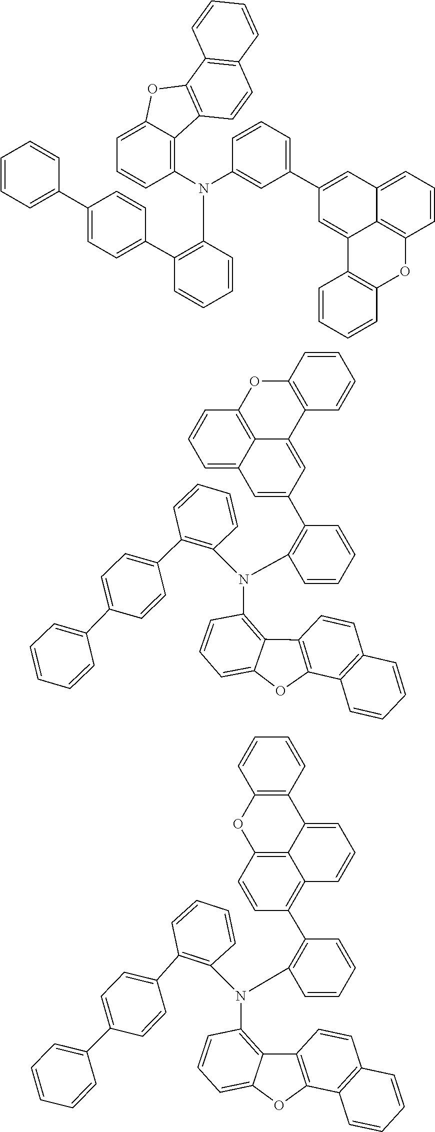

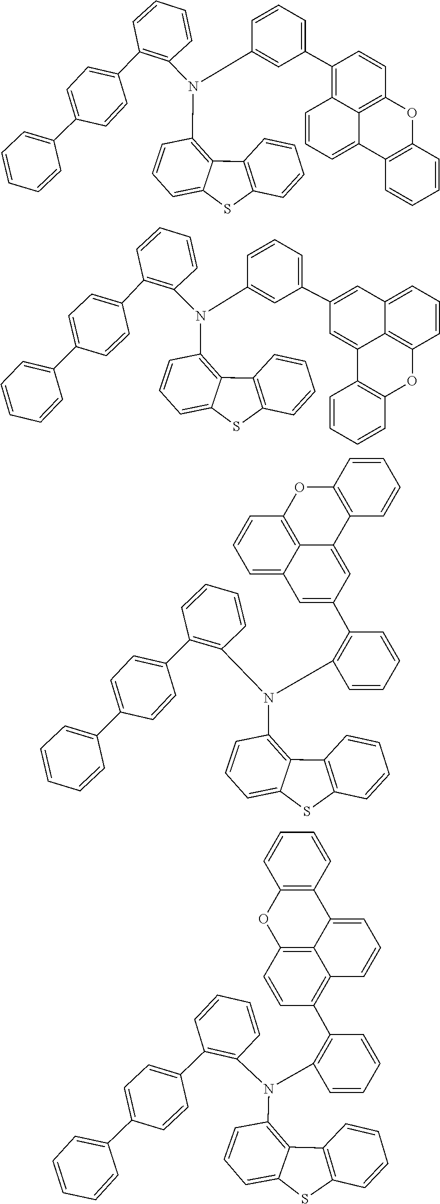

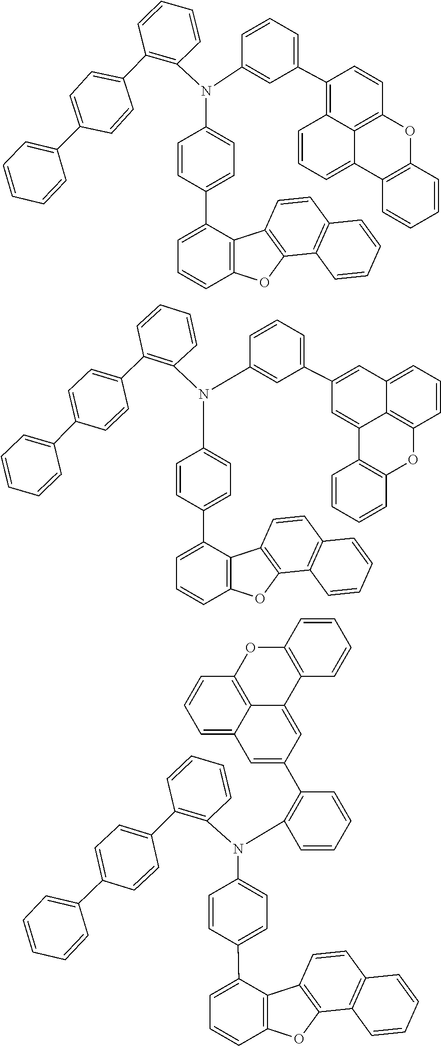

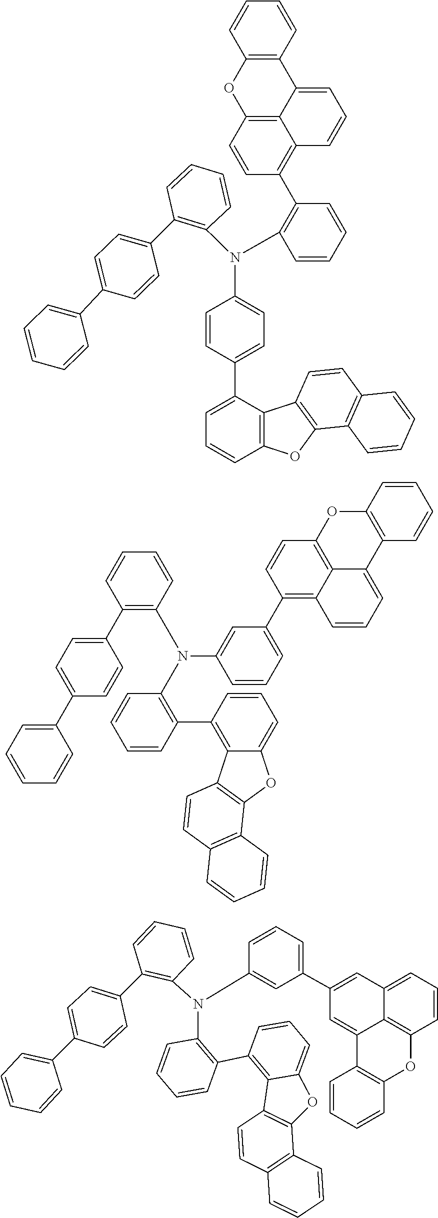

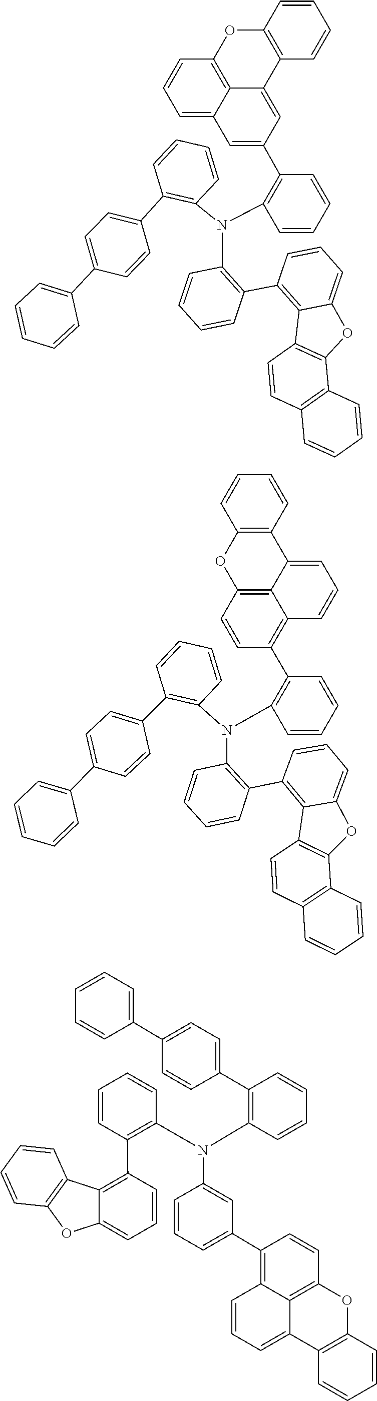

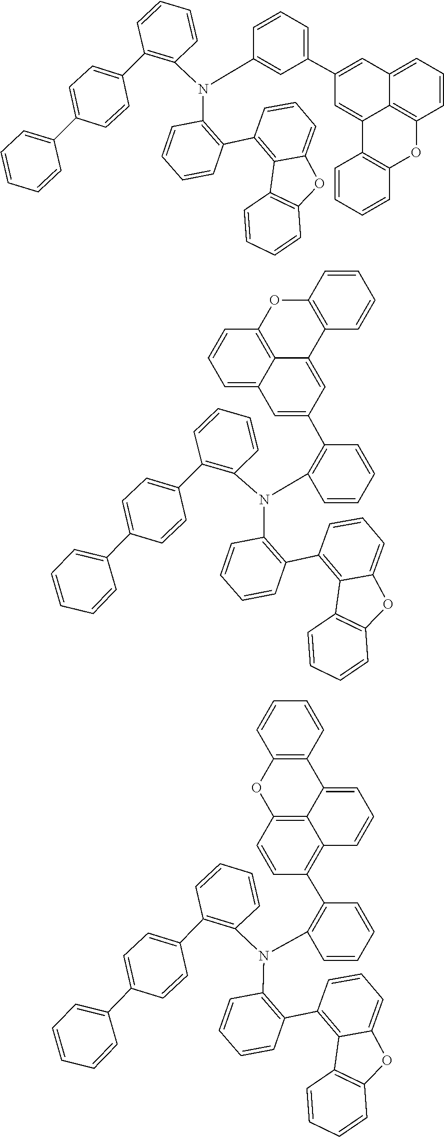

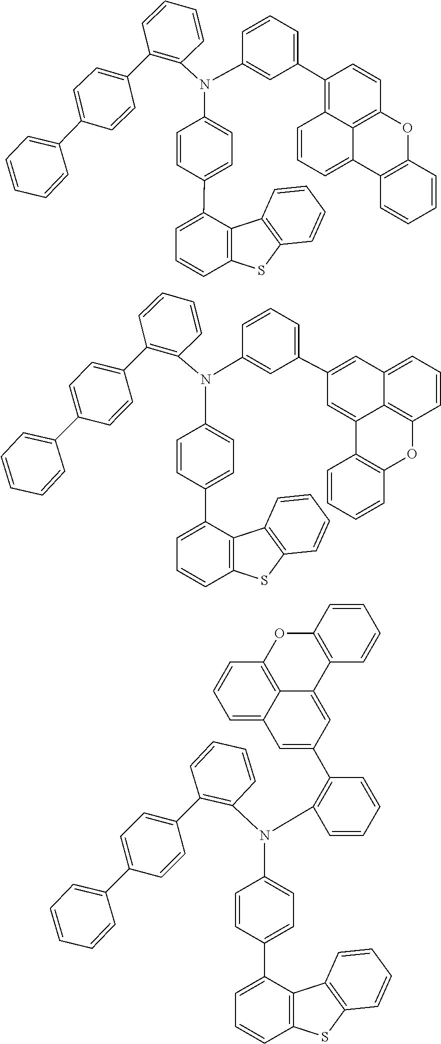

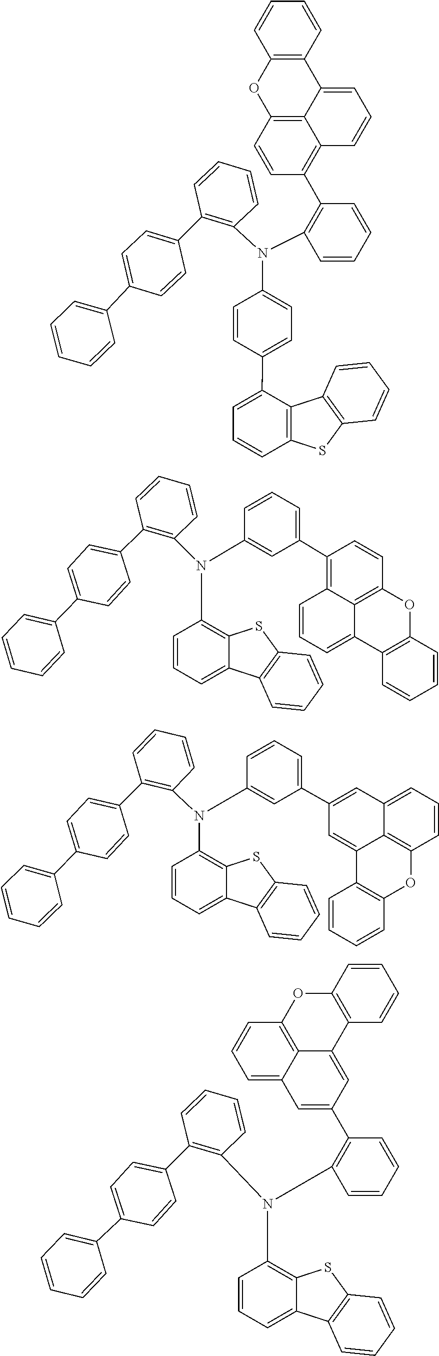

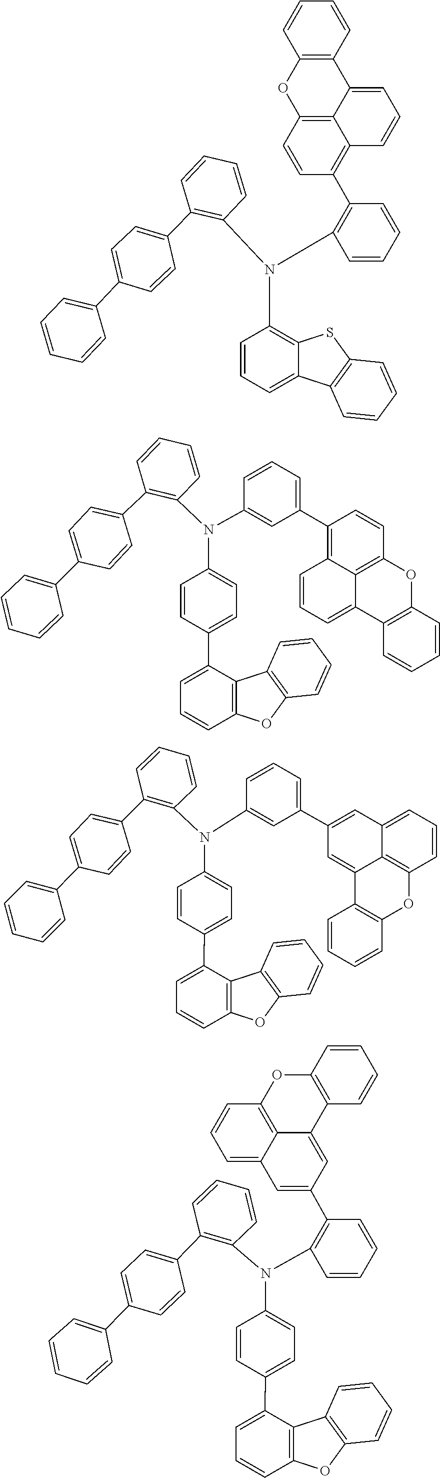

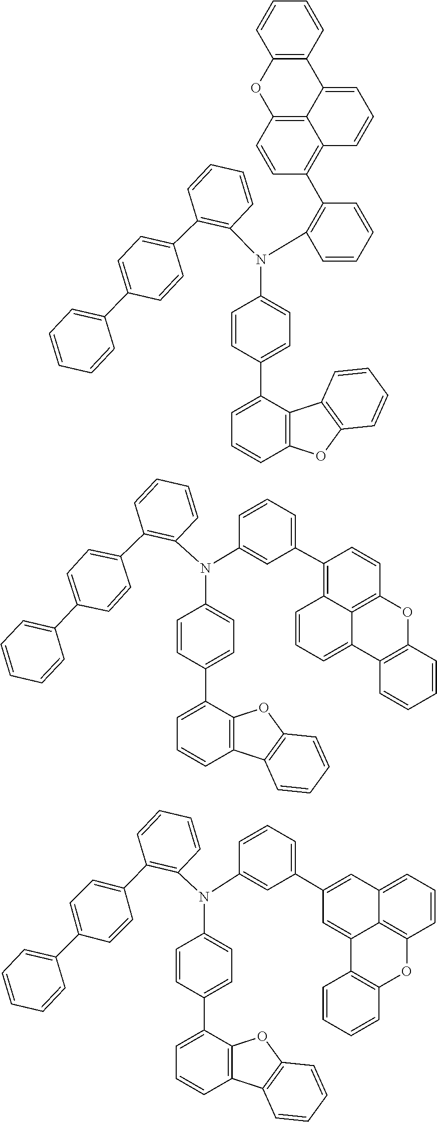

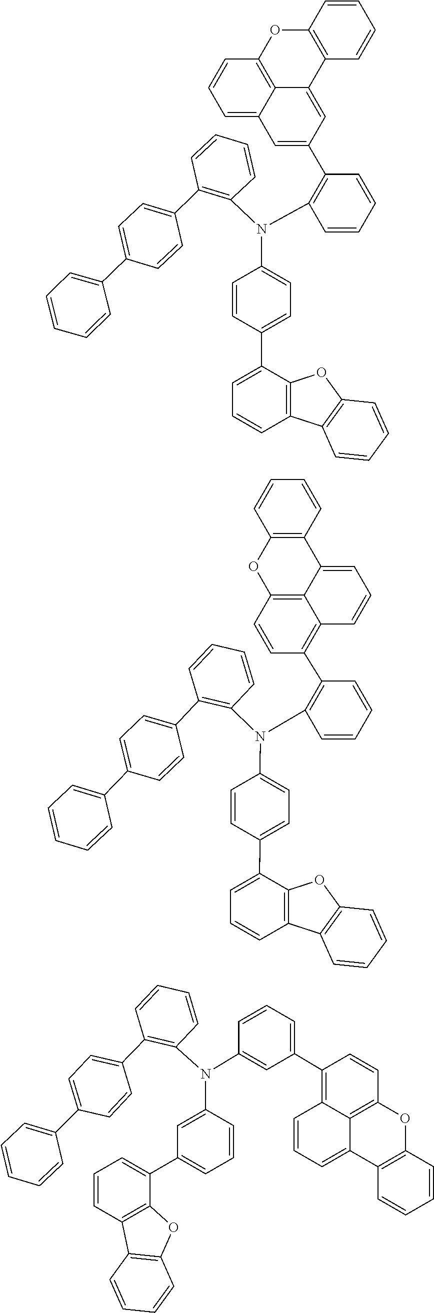

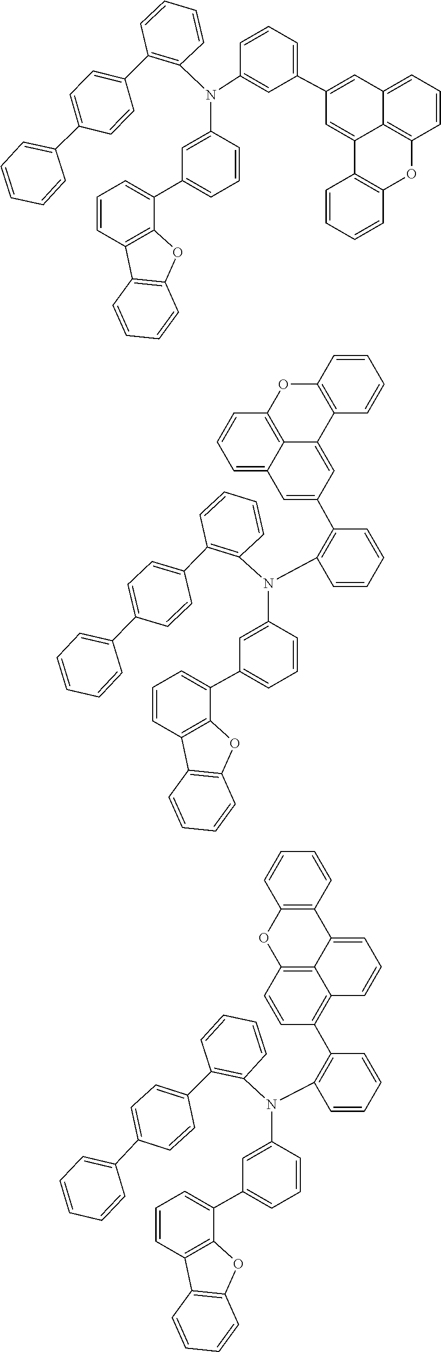 [SMILES: c1ccc(-c2ccc(-c3ccccc3N(c3cccc(-c4cc5c6c(cccc6c4)Oc4ccccc4-5)c3)c3cccc(-c4cccc5c4oc4ccccc45)c3)cc2)cc1 ; c1ccc(-c2ccc(-c3ccccc3N(c3cccc(-c4cccc5c4oc4ccccc45)c3)c3ccccc3-c3cc4c5c(cccc5c3)Oc3ccccc3-4)cc2)cc1 ; c1ccc(-c2ccc(-c3ccccc3N(c3cccc(-c4cccc5c4oc4ccccc45)c3)c3ccccc3-c3ccc4c5c(cccc35)-c3ccccc3O4)cc2)cc1]

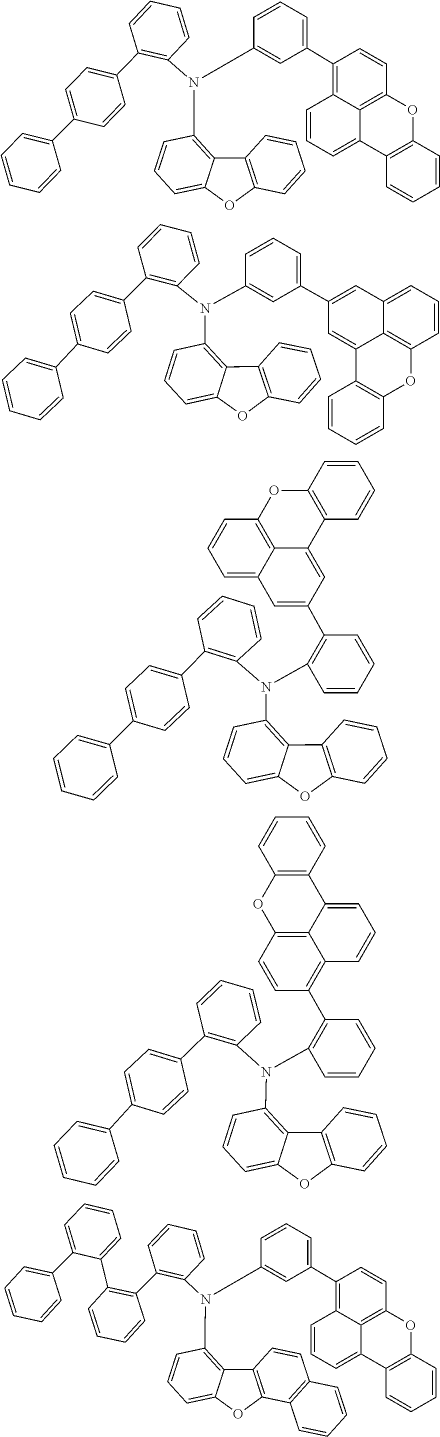

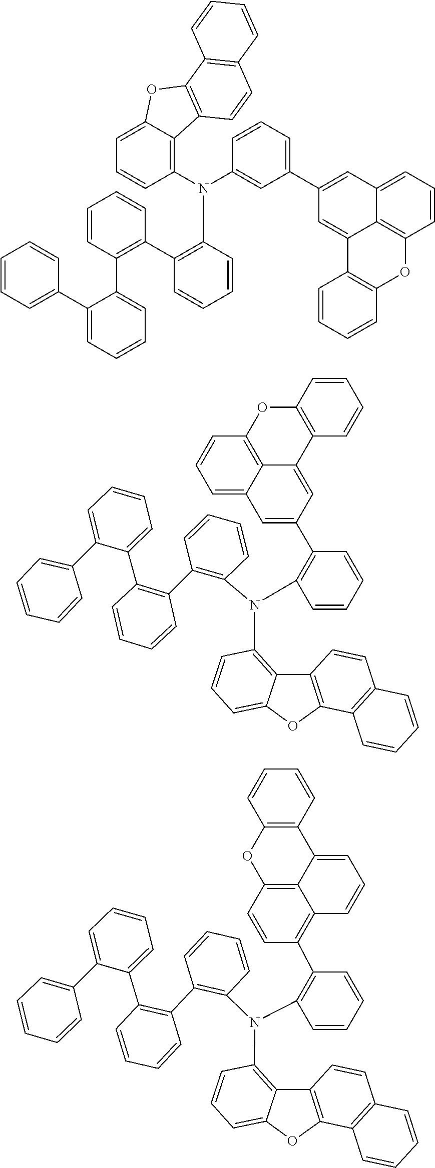

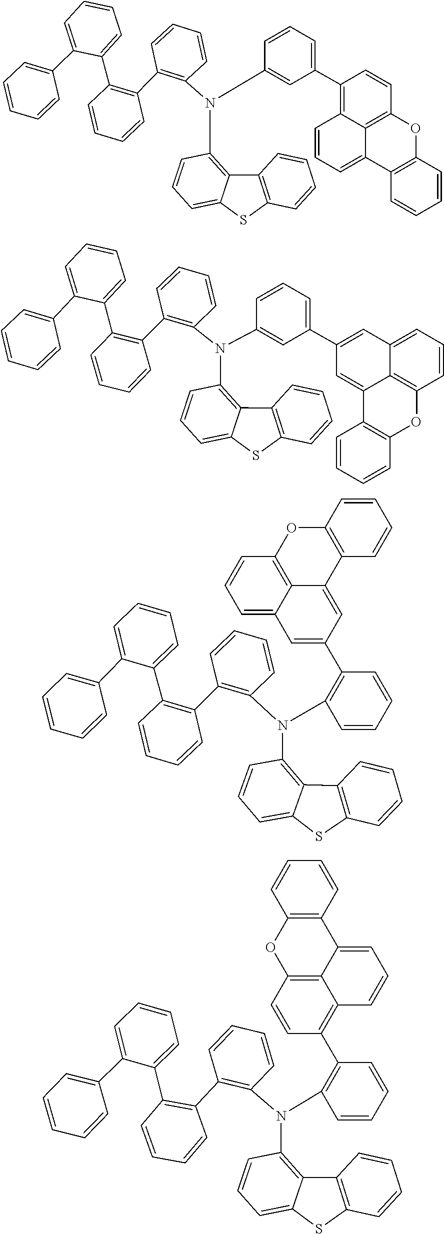

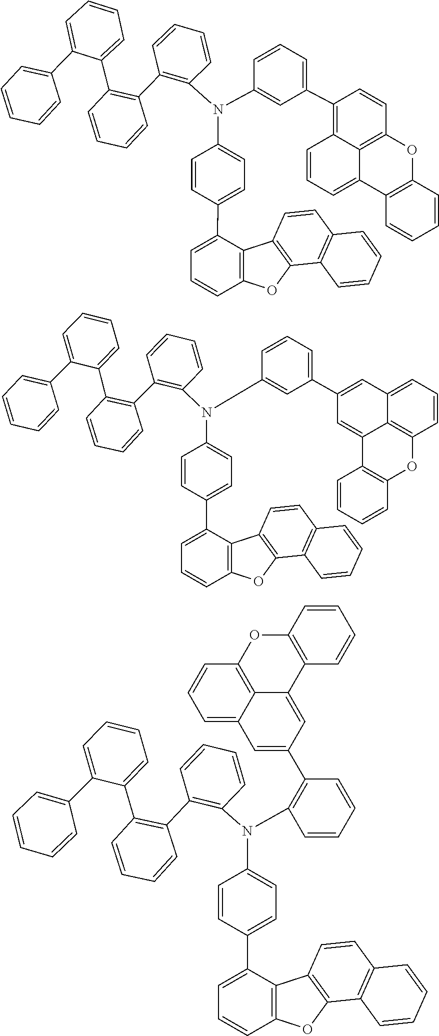

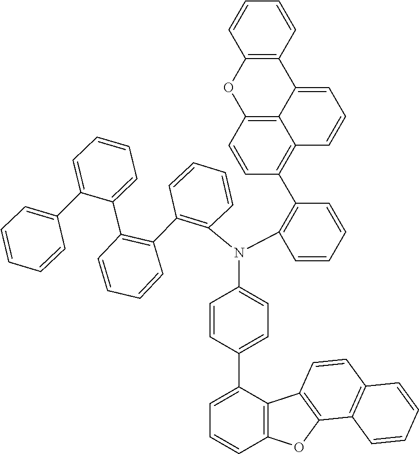 [SMILES: c1ccc(-c2ccccc2-c2ccccc2-c2ccccc2N(c2ccc(-c3cccc4oc5c6ccccc6ccc5c34)cc2)c2ccccc2-c2ccc3c4c(cccc24)-c2ccccc2O3)cc1]

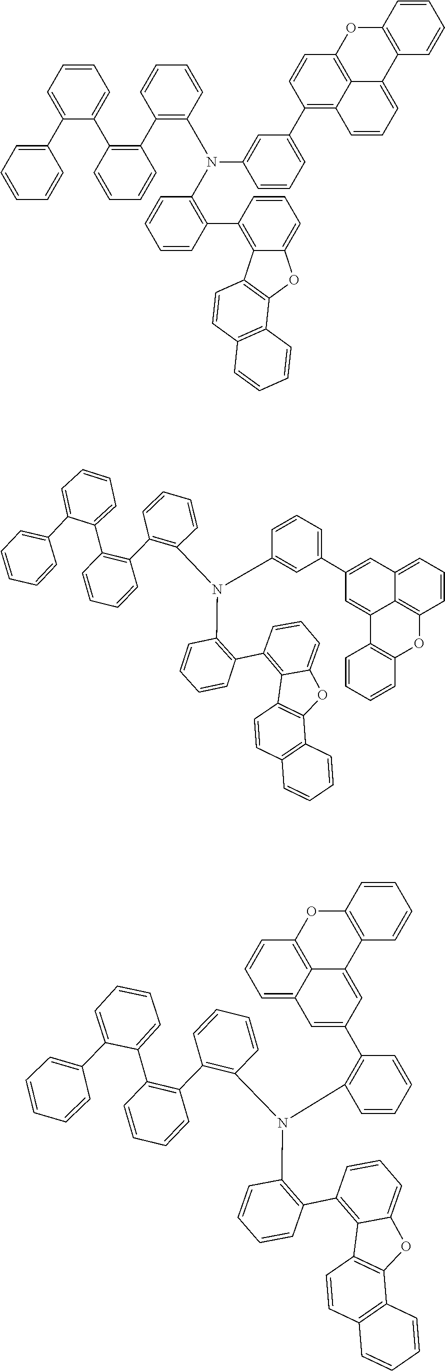 [SMILES: c1ccc(-c2ccccc2-c2ccccc2-c2ccccc2N(c2cccc(-c3ccc4c5c(cccc35)-c3ccccc3O4)c2)c2ccccc2-c2cccc3oc4c5ccccc5ccc4c23)cc1 ; c1ccc(-c2ccccc2-c2ccccc2-c2ccccc2N(c2cccc(-c3cc4c5c(cccc5c3)Oc3ccccc3-4)c2)c2ccccc2-c2cccc3oc4c5ccccc5ccc4c23)cc1 ; c1ccc(-c2ccccc2-c2ccccc2-c2ccccc2N(c2ccccc2-c2cc3c4c(cccc4c2)Oc2ccccc2-3)c2ccccc2-c2cccc3oc4c5ccccc5ccc4c23)cc1]

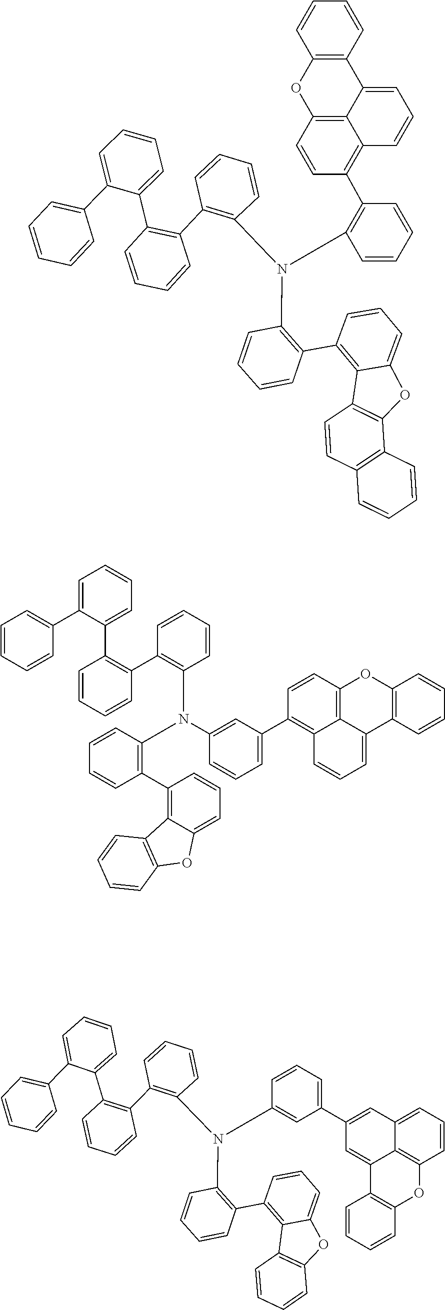 [SMILES: c1ccc(-c2ccccc2-c2ccccc2-c2ccccc2N(c2ccccc2-c2ccc3c4c(cccc24)-c2ccccc2O3)c2ccccc2-c2cccc3oc4c5ccccc5ccc4c23)cc1 ; c1ccc(-c2ccccc2-c2ccccc2-c2ccccc2N(c2cccc(-c3ccc4c5c(cccc35)-c3ccccc3O4)c2)c2ccccc2-c2cccc3oc4ccccc4c23)cc1 ; c1ccc(-c2ccccc2-c2ccccc2-c2ccccc2N(c2cccc(-c3cc4c5c(cccc5c3)Oc3ccccc3-4)c2)c2ccccc2-c2cccc3oc4ccccc4c23)cc1]

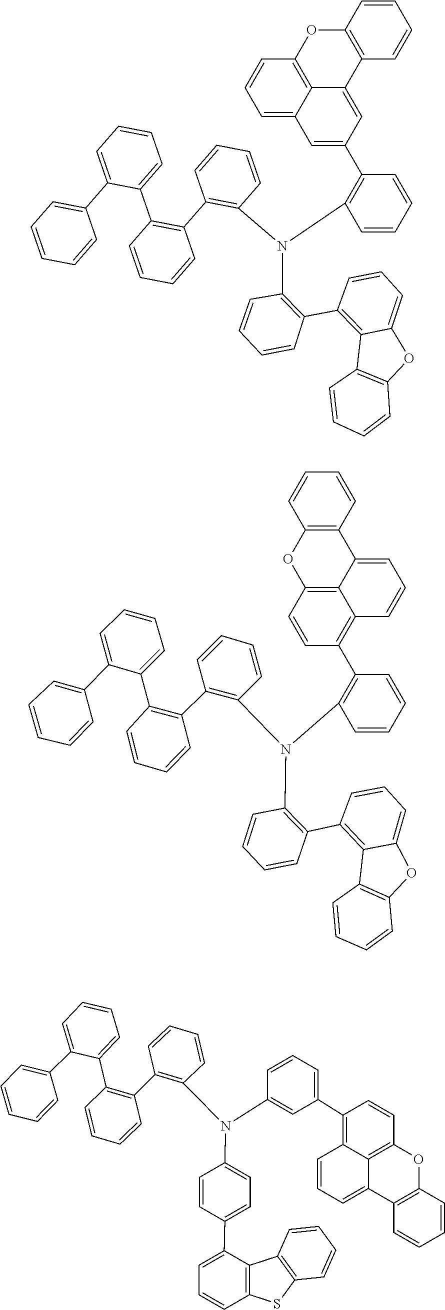

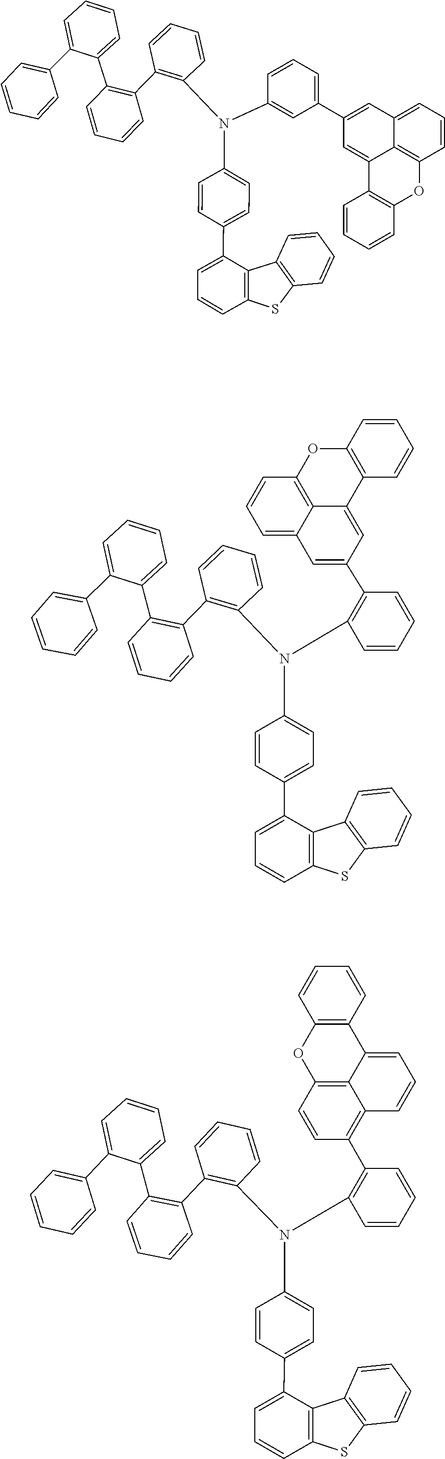 [SMILES: c1ccc(-c2ccccc2-c2ccccc2-c2ccccc2N(c2ccc(-c3cccc4sc5ccccc5c34)cc2)c2cccc(-c3cc4c5c(cccc5c3)Oc3ccccc3-4)c2)cc1 ; c1ccc(-c2ccccc2-c2ccccc2-c2ccccc2N(c2ccc(-c3cccc4sc5ccccc5c34)cc2)c2ccccc2-c2cc3c4c(cccc4c2)Oc2ccccc2-3)cc1 ; c1ccc(-c2ccccc2-c2ccccc2-c2ccccc2N(c2ccc(-c3cccc4sc5ccccc5c34)cc2)c2ccccc2-c2ccc3c4c(cccc24)-c2ccccc2O3)cc1]

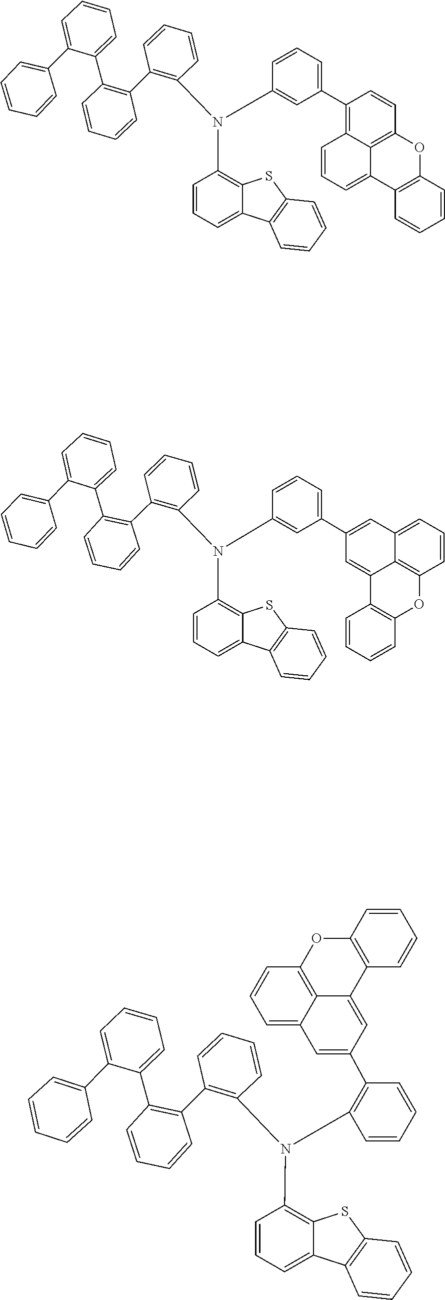 [SMILES: c1ccc(-c2ccccc2-c2ccccc2-c2ccccc2N(c2cccc(-c3ccc4c5c(cccc35)-c3ccccc3O4)c2)c2cccc3c2sc2ccccc23)cc1 ; c1ccc(-c2ccccc2-c2ccccc2-c2ccccc2N(c2cccc(-c3cc4c5c(cccc5c3)Oc3ccccc3-4)c2)c2cccc3c2sc2ccccc23)cc1 ; c1ccc(-c2ccccc2-c2ccccc2-c2ccccc2N(c2ccccc2-c2cc3c4c(cccc4c2)Oc2ccccc2-3)c2cccc3c2sc2ccccc23)cc1]

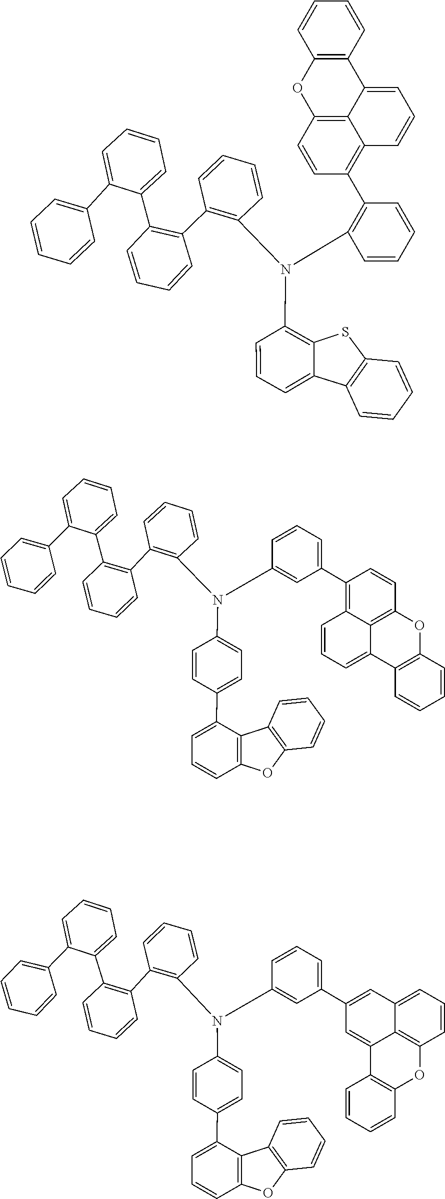 [SMILES: c1ccc(-c2ccccc2-c2ccccc2-c2ccccc2N(c2ccccc2-c2ccc3c4c(cccc24)-c2ccccc2O3)c2cccc3c2sc2ccccc23)cc1 ; c1ccc(-c2ccccc2-c2ccccc2-c2ccccc2N(c2ccc(-c3cccc4oc5ccccc5c34)cc2)c2cccc(-c3ccc4c5c(cccc35)-c3ccccc3O4)c2)cc1 ; c1ccc(-c2ccccc2-c2ccccc2-c2ccccc2N(c2ccc(-c3cccc4oc5ccccc5c34)cc2)c2cccc(-c3cc4c5c(cccc5c3)Oc3ccccc3-4)c2)cc1]

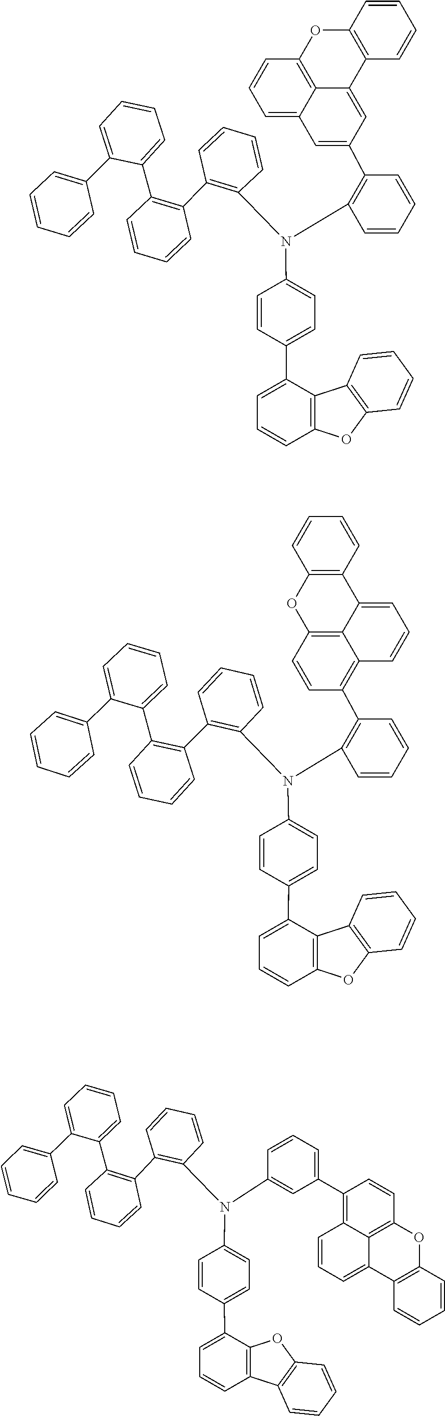 [SMILES: c1ccc(-c2ccccc2-c2ccccc2-c2ccccc2N(c2ccc(-c3cccc4oc5ccccc5c34)cc2)c2ccccc2-c2cc3c4c(cccc4c2)Oc2ccccc2-3)cc1 ; c1ccc(-c2ccccc2-c2ccccc2-c2ccccc2N(c2ccc(-c3cccc4oc5ccccc5c34)cc2)c2ccccc2-c2ccc3c4c(cccc24)-c2ccccc2O3)cc1 ; c1ccc(-c2ccccc2-c2ccccc2-c2ccccc2N(c2ccc(-c3cccc4c3oc3ccccc34)cc2)c2cccc(-c3ccc4c5c(cccc35)-c3ccccc3O4)c2)cc1]

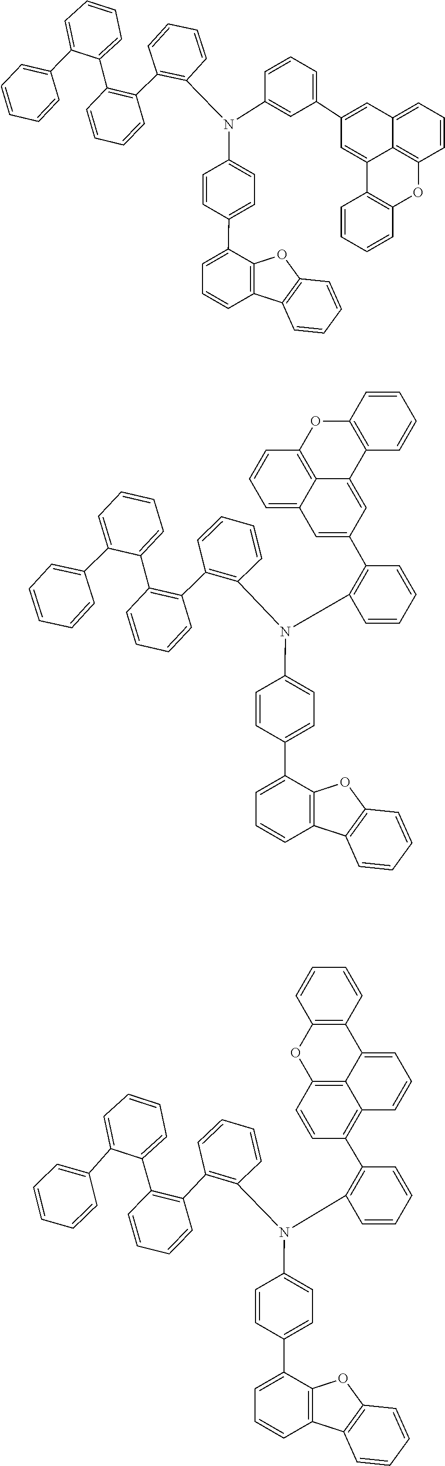 [SMILES: c1ccc(-c2ccccc2-c2ccccc2-c2ccccc2N(c2ccc(-c3cccc4c3oc3ccccc34)cc2)c2cccc(-c3cc4c5c(cccc5c3)Oc3ccccc3-4)c2)cc1 ; c1ccc(-c2ccccc2-c2ccccc2-c2ccccc2N(c2ccc(-c3cccc4c3oc3ccccc34)cc2)c2ccccc2-c2cc3c4c(cccc4c2)Oc2ccccc2-3)cc1 ; c1ccc(-c2ccccc2-c2ccccc2-c2ccccc2N(c2ccc(-c3cccc4c3oc3ccccc34)cc2)c2ccccc2-c2ccc3c4c(cccc24)-c2ccccc2O3)cc1]

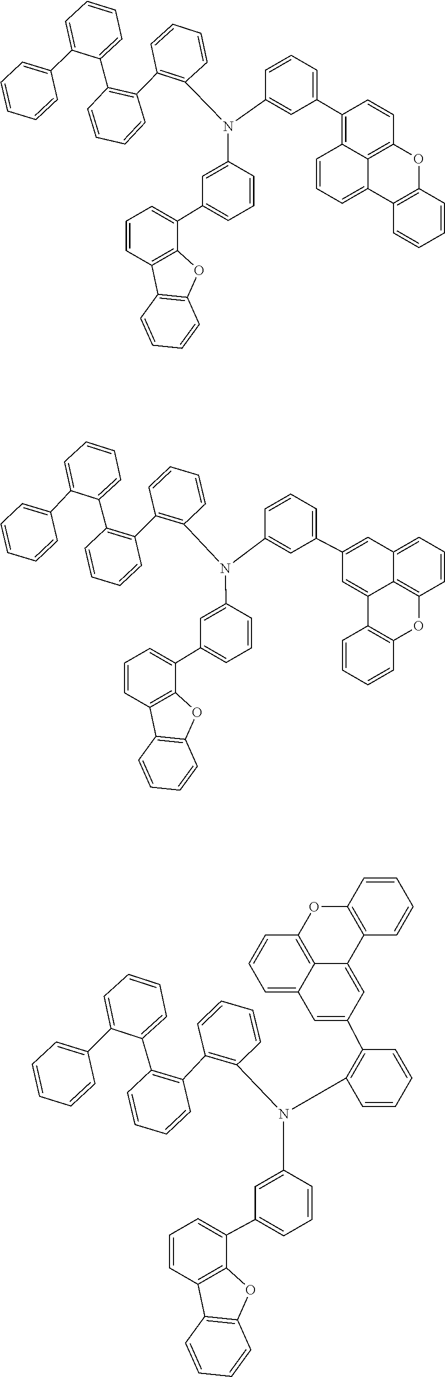 [SMILES: c1ccc(-c2ccccc2-c2ccccc2-c2ccccc2N(c2cccc(-c3ccc4c5c(cccc35)-c3ccccc3O4)c2)c2cccc(-c3cccc4c3oc3ccccc34)c2)cc1 ; c1ccc(-c2ccccc2-c2ccccc2-c2ccccc2N(c2cccc(-c3cc4c5c(cccc5c3)Oc3ccccc3-4)c2)c2cccc(-c3cccc4c3oc3ccccc34)c2)cc1 ; c1ccc(-c2ccccc2-c2ccccc2-c2ccccc2N(c2cccc(-c3cccc4c3oc3ccccc34)c2)c2ccccc2-c2cc3c4c(cccc4c2)Oc2ccccc2-3)cc1]

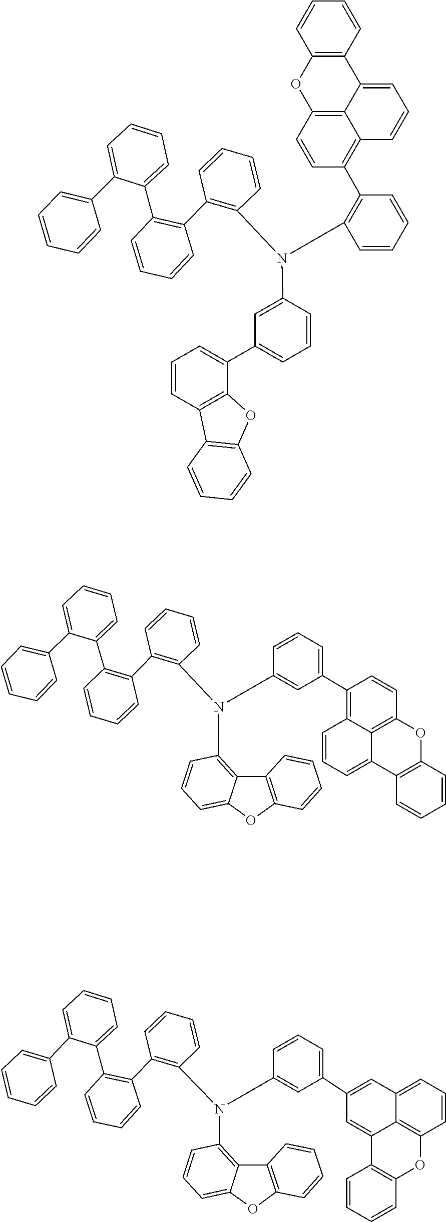 [SMILES: c1ccc(-c2ccccc2-c2ccccc2-c2ccccc2N(c2cccc(-c3cccc4c3oc3ccccc34)c2)c2ccccc2-c2ccc3c4c(cccc24)-c2ccccc2O3)cc1 ; c1ccc(-c2ccccc2-c2ccccc2-c2ccccc2N(c2cccc(-c3ccc4c5c(cccc35)-c3ccccc3O4)c2)c2cccc3oc4ccccc4c23)cc1 ; c1ccc(-c2ccccc2-c2ccccc2-c2ccccc2N(c2cccc(-c3cc4c5c(cccc5c3)Oc3ccccc3-4)c2)c2cccc3oc4ccccc4c23)cc1]

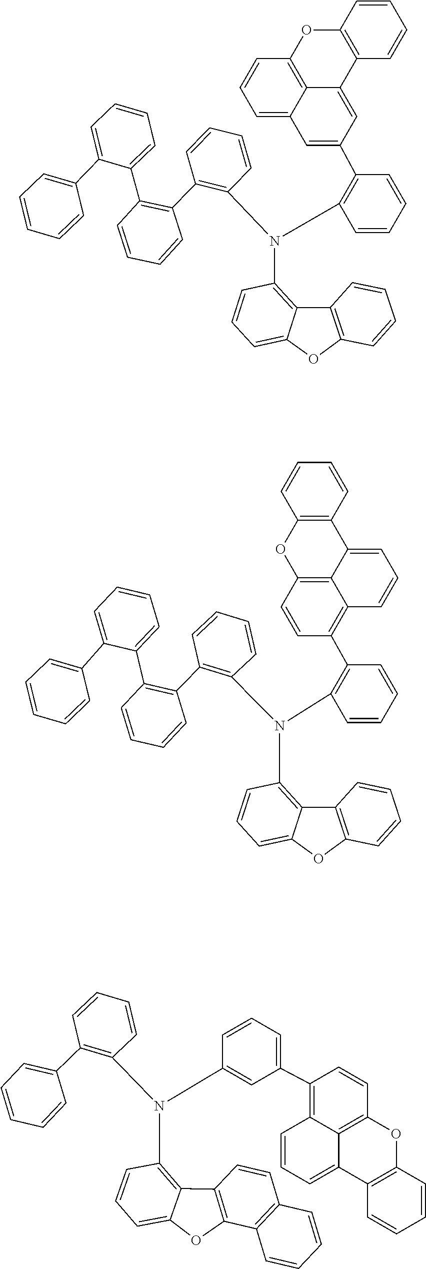 [SMILES: c1ccc(-c2ccccc2-c2ccccc2-c2ccccc2N(c2ccccc2-c2cc3c4c(cccc4c2)Oc2ccccc2-3)c2cccc3oc4ccccc4c23)cc1 ; c1ccc(-c2ccccc2-c2ccccc2-c2ccccc2N(c2ccccc2-c2ccc3c4c(cccc24)-c2ccccc2O3)c2cccc3oc4ccccc4c23)cc1 ; c1ccc(-c2ccccc2N(c2cccc(-c3ccc4c5c(cccc35)-c3ccccc3O4)c2)c2cccc3oc4c5ccccc5ccc4c23)cc1]

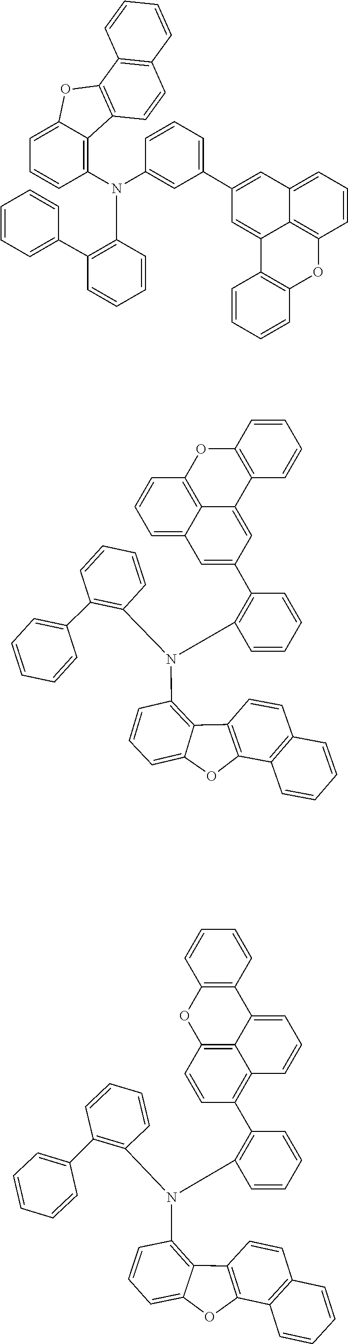 [SMILES: c1ccc(-c2ccccc2N(c2cccc(-c3cc4c5c(cccc5c3)Oc3ccccc3-4)c2)c2cccc3oc4c5ccccc5ccc4c23)cc1 ; c1ccc(-c2ccccc2N(c2ccccc2-c2cc3c4c(cccc4c2)Oc2ccccc2-3)c2cccc3oc4c5ccccc5ccc4c23)cc1 ; c1ccc(-c2ccccc2N(c2ccccc2-c2ccc3c4c(cccc24)-c2ccccc2O3)c2cccc3oc4c5ccccc5ccc4c23)cc1]

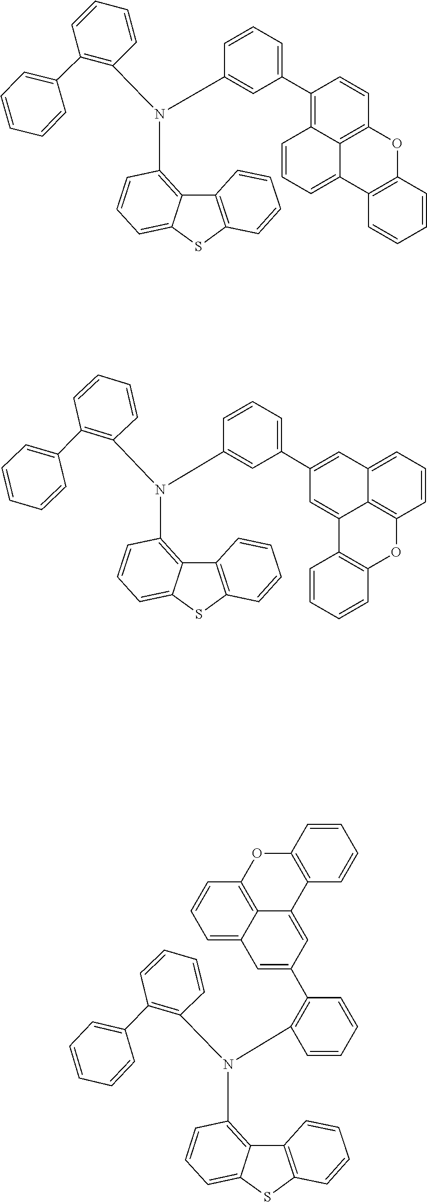 [SMILES: c1ccc(-c2ccccc2N(c2cccc(-c3ccc4c5c(cccc35)-c3ccccc3O4)c2)c2cccc3sc4ccccc4c23)cc1 ; c1ccc(-c2ccccc2N(c2cccc(-c3cc4c5c(cccc5c3)Oc3ccccc3-4)c2)c2cccc3sc4ccccc4c23)cc1 ; c1ccc(-c2ccccc2N(c2ccccc2-c2cc3c4c(cccc4c2)Oc2ccccc2-3)c2cccc3sc4ccccc4c23)cc1]

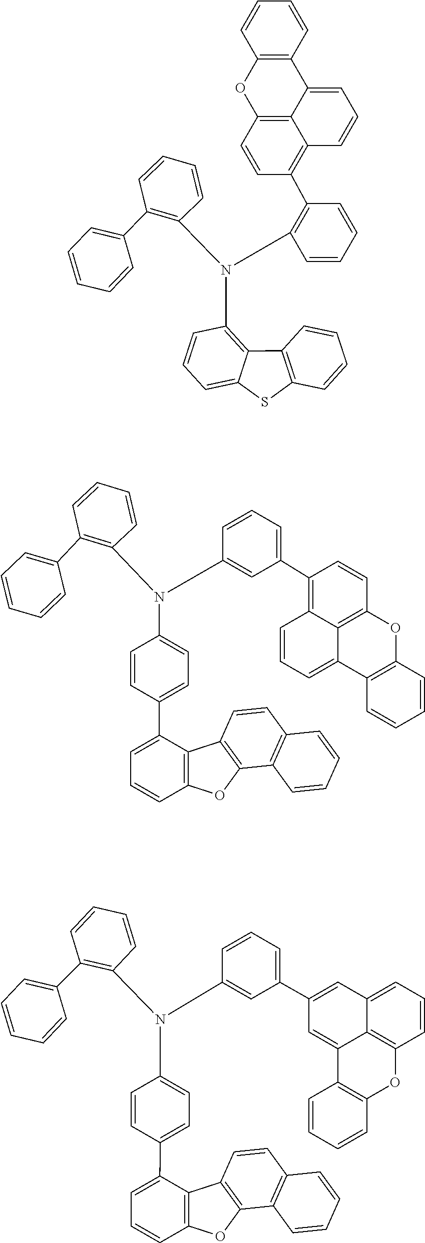 [SMILES: c1ccc(-c2ccccc2N(c2ccccc2-c2ccc3c4c(cccc24)-c2ccccc2O3)c2cccc3sc4ccccc4c23)cc1 ; c1ccc(-c2ccccc2N(c2ccc(-c3cccc4oc5c6ccccc6ccc5c34)cc2)c2cccc(-c3ccc4c5c(cccc35)-c3ccccc3O4)c2)cc1 ; c1ccc(-c2ccccc2N(c2ccc(-c3cccc4oc5c6ccccc6ccc5c34)cc2)c2cccc(-c3cc4c5c(cccc5c3)Oc3ccccc3-4)c2)cc1]

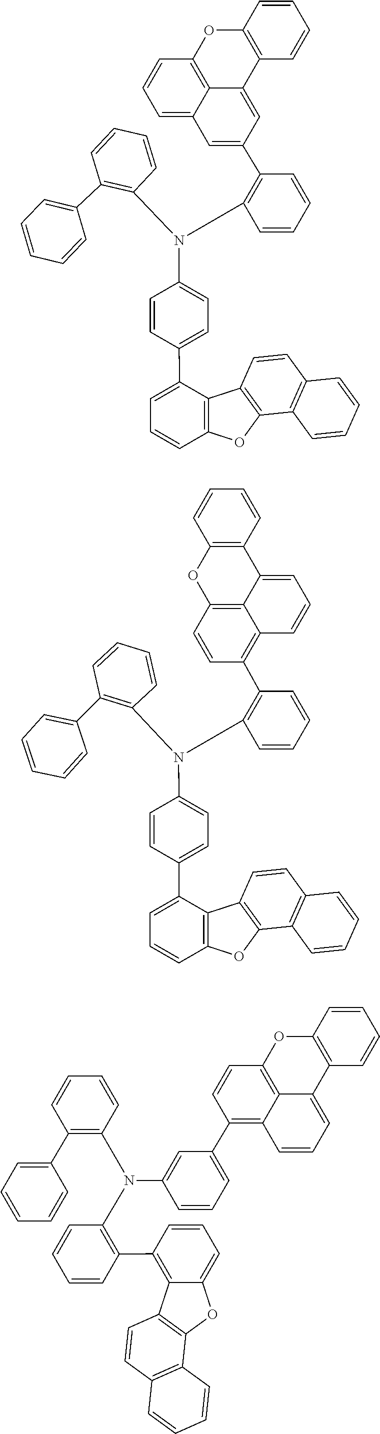 [SMILES: c1ccc(-c2ccccc2N(c2ccc(-c3cccc4oc5c6ccccc6ccc5c34)cc2)c2ccccc2-c2cc3c4c(cccc4c2)Oc2ccccc2-3)cc1 ; c1ccc(-c2ccccc2N(c2ccc(-c3cccc4oc5c6ccccc6ccc5c34)cc2)c2ccccc2-c2ccc3c4c(cccc24)-c2ccccc2O3)cc1 ; c1ccc(-c2ccccc2N(c2cccc(-c3ccc4c5c(cccc35)-c3ccccc3O4)c2)c2ccccc2-c2cccc3oc4c5ccccc5ccc4c23)cc1]

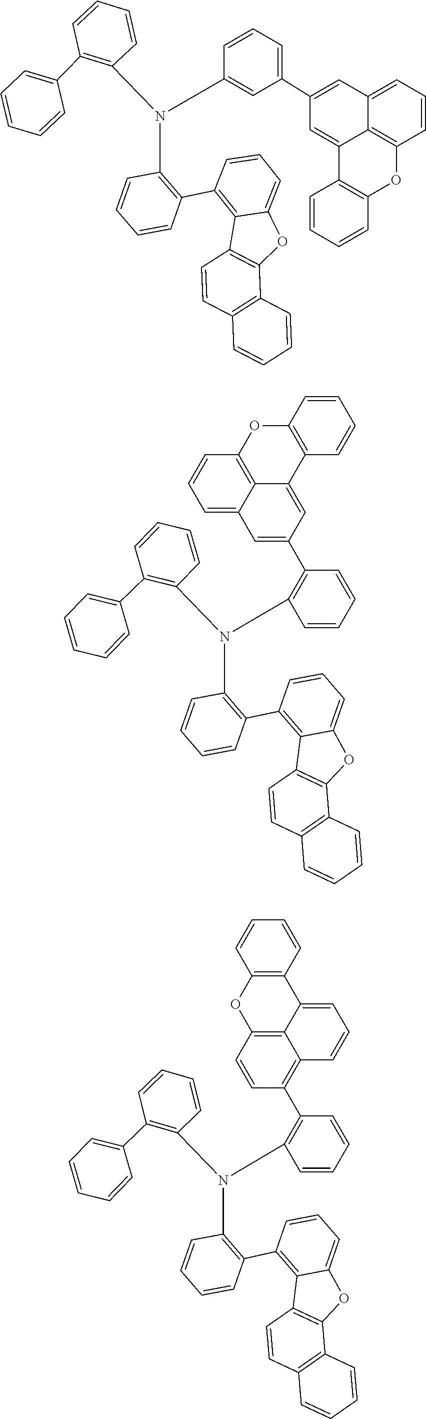 [SMILES: c1ccc(-c2ccccc2N(c2cccc(-c3cc4c5c(cccc5c3)Oc3ccccc3-4)c2)c2ccccc2-c2cccc3oc4c5ccccc5ccc4c23)cc1 ; c1ccc(-c2ccccc2N(c2ccccc2-c2cc3c4c(cccc4c2)Oc2ccccc2-3)c2ccccc2-c2cccc3oc4c5ccccc5ccc4c23)cc1 ; c1ccc(-c2ccccc2N(c2ccccc2-c2ccc3c4c(cccc24)-c2ccccc2O3)c2ccccc2-c2cccc3oc4c5ccccc5ccc4c23)cc1]

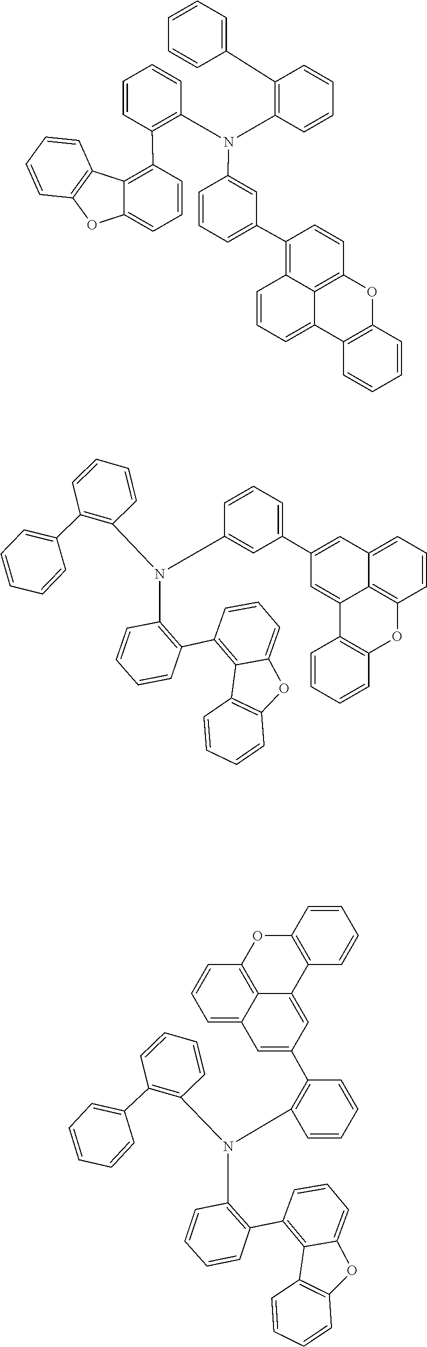 [SMILES: c1ccc(-c2ccccc2N(c2cccc(-c3ccc4c5c(cccc35)-c3ccccc3O4)c2)c2ccccc2-c2cccc3oc4ccccc4c23)cc1 ; c1ccc(-c2ccccc2N(c2cccc(-c3cc4c5c(cccc5c3)Oc3ccccc3-4)c2)c2ccccc2-c2cccc3oc4ccccc4c23)cc1 ; c1ccc(-c2ccccc2N(c2ccccc2-c2cc3c4c(cccc4c2)Oc2ccccc2-3)c2ccccc2-c2cccc3oc4ccccc4c23)cc1]

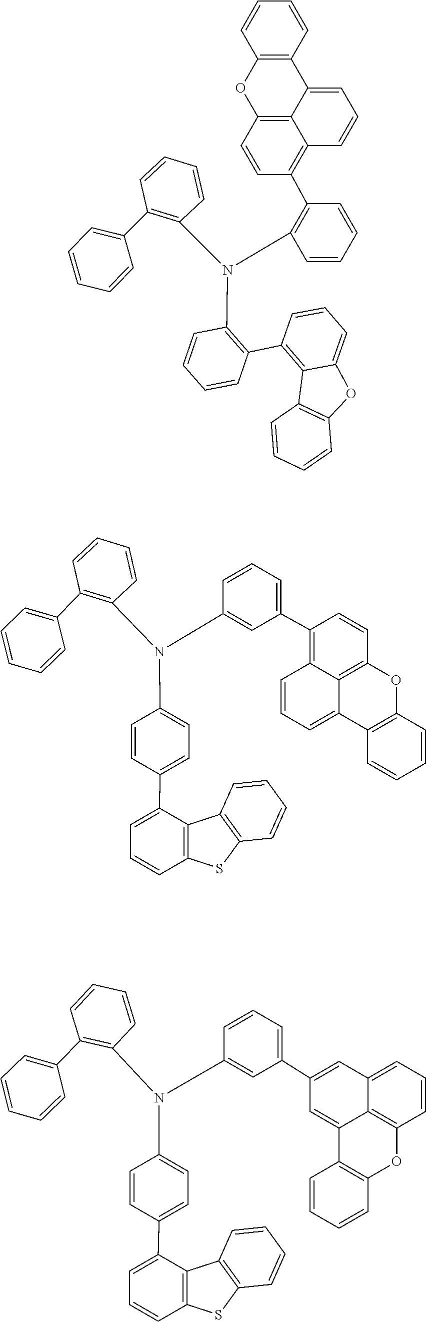 [SMILES: c1ccc(-c2ccccc2N(c2ccccc2-c2ccc3c4c(cccc24)-c2ccccc2O3)c2ccccc2-c2cccc3oc4ccccc4c23)cc1 ; c1ccc(-c2ccccc2N(c2ccc(-c3cccc4sc5ccccc5c34)cc2)c2cccc(-c3ccc4c5c(cccc35)-c3ccccc3O4)c2)cc1 ; c1ccc(-c2ccccc2N(c2ccc(-c3cccc4sc5ccccc5c34)cc2)c2cccc(-c3cc4c5c(cccc5c3)Oc3ccccc3-4)c2)cc1]

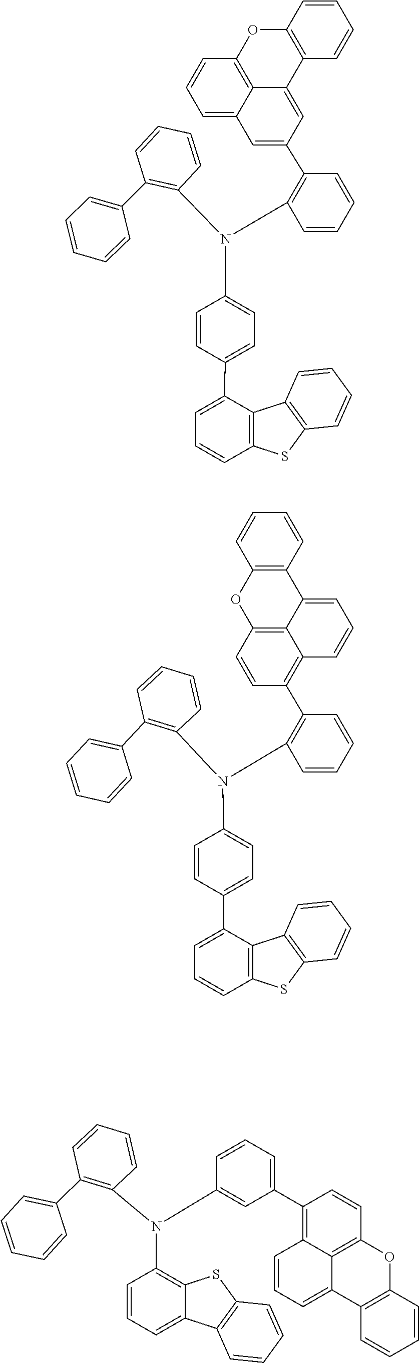 [SMILES: c1ccc(-c2ccccc2N(c2ccc(-c3cccc4sc5ccccc5c34)cc2)c2ccccc2-c2cc3c4c(cccc4c2)Oc2ccccc2-3)cc1 ; c1ccc(-c2ccccc2N(c2ccc(-c3cccc4sc5ccccc5c34)cc2)c2ccccc2-c2ccc3c4c(cccc24)-c2ccccc2O3)cc1 ; c1ccc(-c2ccccc2N(c2cccc(-c3ccc4c5c(cccc35)-c3ccccc3O4)c2)c2cccc3c2sc2ccccc23)cc1]

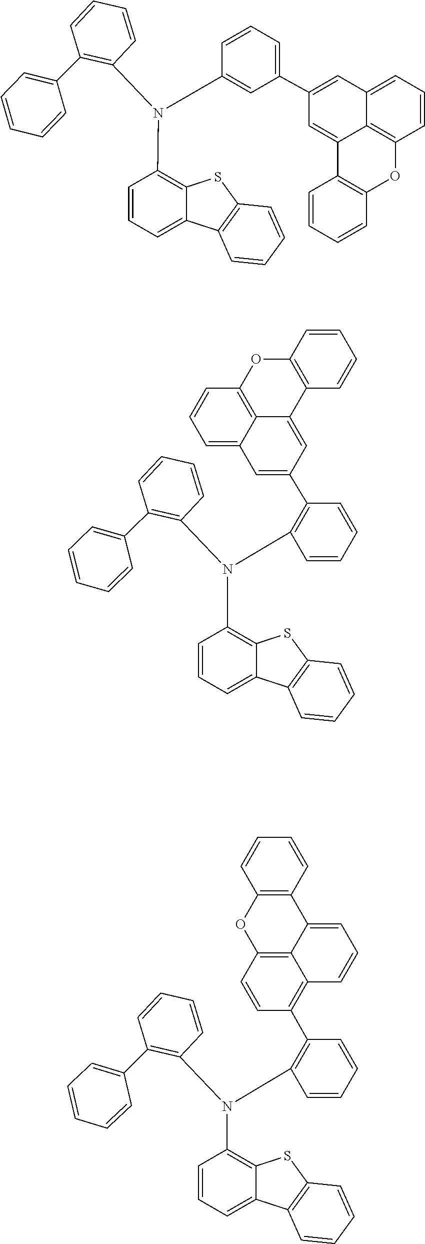 [SMILES: c1ccc(-c2ccccc2N(c2cccc(-c3cc4c5c(cccc5c3)Oc3ccccc3-4)c2)c2cccc3c2sc2ccccc23)cc1 ; c1ccc(-c2ccccc2N(c2ccccc2-c2cc3c4c(cccc4c2)Oc2ccccc2-3)c2cccc3c2sc2ccccc23)cc1 ; c1ccc(-c2ccccc2N(c2ccccc2-c2ccc3c4c(cccc24)-c2ccccc2O3)c2cccc3c2sc2ccccc23)cc1]

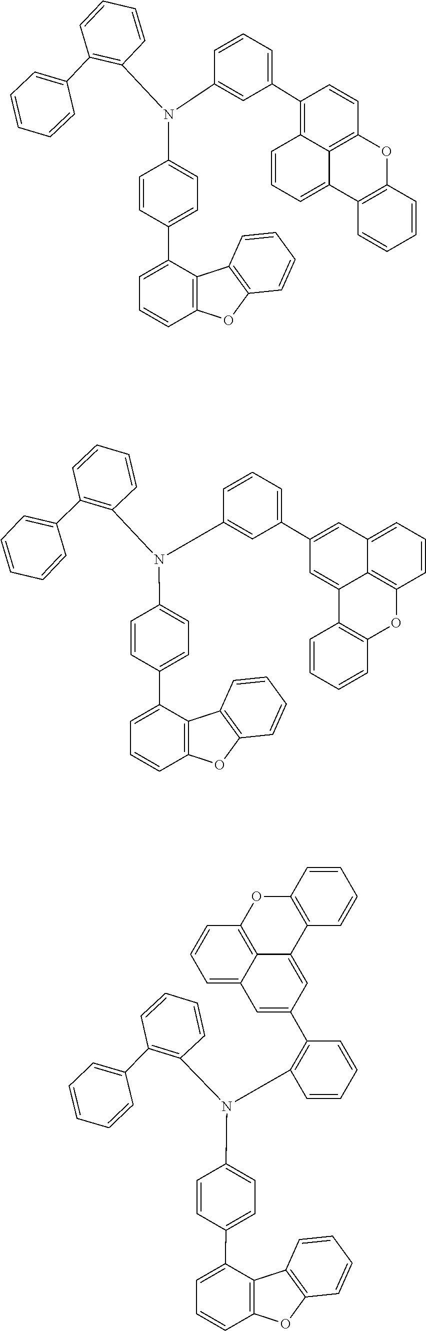 [SMILES: c1ccc(-c2ccccc2N(c2ccc(-c3cccc4oc5ccccc5c34)cc2)c2cccc(-c3ccc4c5c(cccc35)-c3ccccc3O4)c2)cc1 ; c1ccc(-c2ccccc2N(c2ccc(-c3cccc4oc5ccccc5c34)cc2)c2cccc(-c3cc4c5c(cccc5c3)Oc3ccccc3-4)c2)cc1 ; c1ccc(-c2ccccc2N(c2ccc(-c3cccc4oc5ccccc5c34)cc2)c2ccccc2-c2cc3c4c(cccc4c2)Oc2ccccc2-3)cc1]

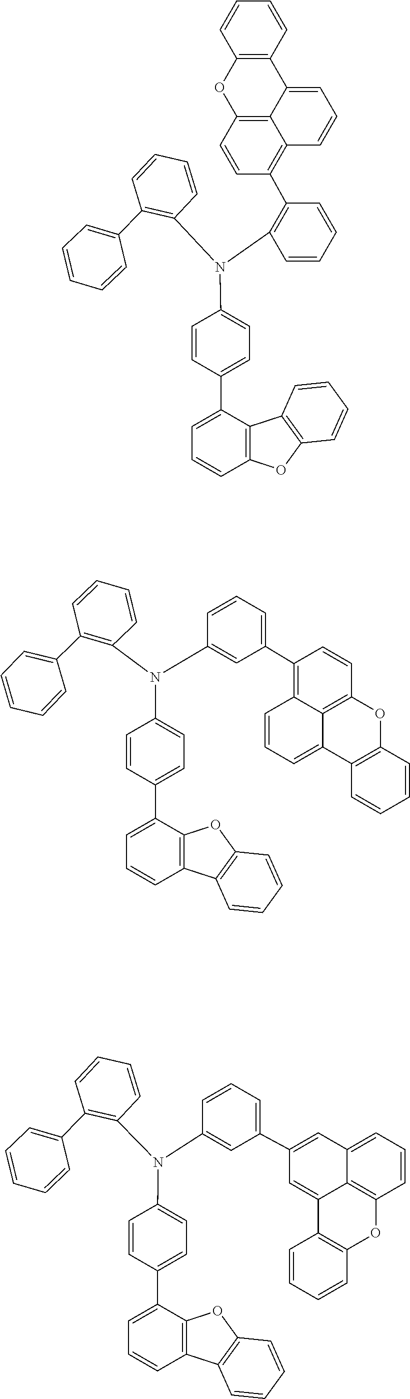 [SMILES: c1ccc(-c2ccccc2N(c2ccc(-c3cccc4oc5ccccc5c34)cc2)c2ccccc2-c2ccc3c4c(cccc24)-c2ccccc2O3)cc1 ; c1ccc(-c2ccccc2N(c2ccc(-c3cccc4c3oc3ccccc34)cc2)c2cccc(-c3ccc4c5c(cccc35)-c3ccccc3O4)c2)cc1 ; c1ccc(-c2ccccc2N(c2ccc(-c3cccc4c3oc3ccccc34)cc2)c2cccc(-c3cc4c5c(cccc5c3)Oc3ccccc3-4)c2)cc1]

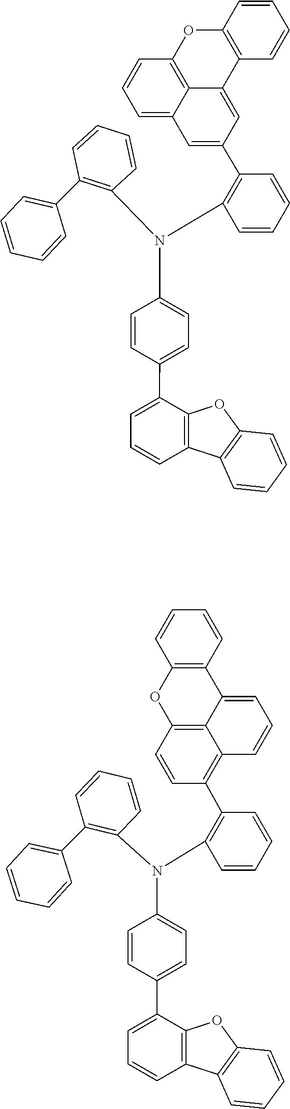 [SMILES: c1ccc(-c2ccccc2N(c2ccc(-c3cccc4c3oc3ccccc34)cc2)c2ccccc2-c2cc3c4c(cccc4c2)Oc2ccccc2-3)cc1 ; c1ccc(-c2ccccc2N(c2ccc(-c3cccc4c3oc3ccccc34)cc2)c2ccccc2-c2ccc3c4c(cccc24)-c2ccccc2O3)cc1]

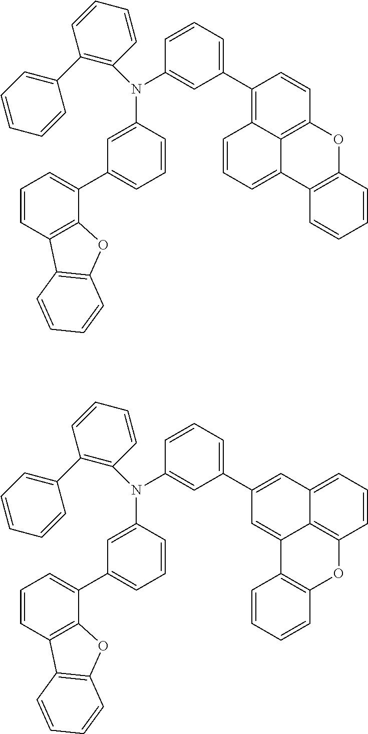 [SMILES: c1ccc(-c2ccccc2N(c2cccc(-c3ccc4c5c(cccc35)-c3ccccc3O4)c2)c2cccc(-c3cccc4c3oc3ccccc34)c2)cc1 ; c1ccc(-c2ccccc2N(c2cccc(-c3cc4c5c(cccc5c3)Oc3ccccc3-4)c2)c2cccc(-c3cccc4c3oc3ccccc34)c2)cc1]

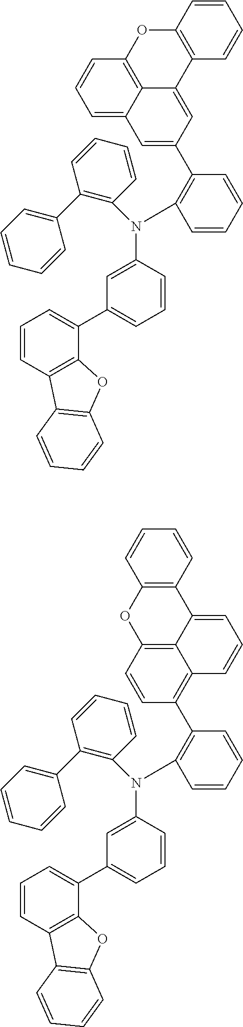 [SMILES: c1ccc(-c2ccccc2N(c2cccc(-c3cccc4c3oc3ccccc34)c2)c2ccccc2-c2cc3c4c(cccc4c2)Oc2ccccc2-3)cc1 ; c1ccc(-c2ccccc2N(c2cccc(-c3cccc4c3oc3ccccc34)c2)c2ccccc2-c2ccc3c4c(cccc24)-c2ccccc2O3)cc1]

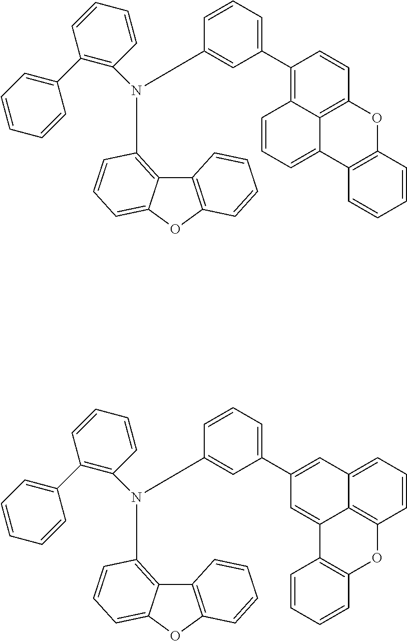 [SMILES: c1ccc(-c2ccccc2N(c2cccc(-c3ccc4c5c(cccc35)-c3ccccc3O4)c2)c2cccc3oc4ccccc4c23)cc1 ; c1ccc(-c2ccccc2N(c2cccc(-c3cc4c5c(cccc5c3)Oc3ccccc3-4)c2)c2cccc3oc4ccccc4c23)cc1]

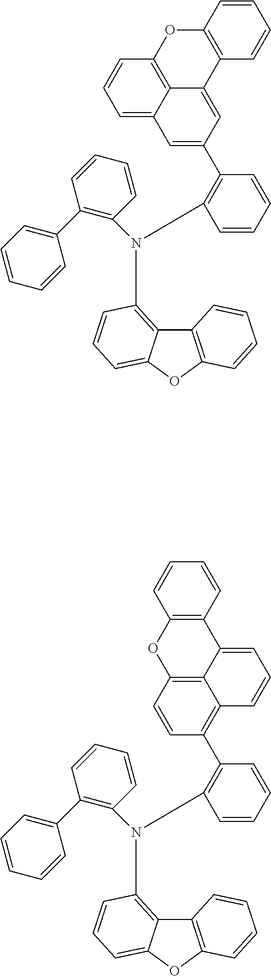 [SMILES: c1ccc(-c2ccccc2N(c2ccccc2-c2cc3c4c(cccc4c2)Oc2ccccc2-3)c2cccc3oc4ccccc4c23)cc1 ; c1ccc(-c2ccccc2N(c2ccccc2-c2ccc3c4c(cccc24)-c2ccccc2O3)c2cccc3oc4ccccc4c23)cc1]

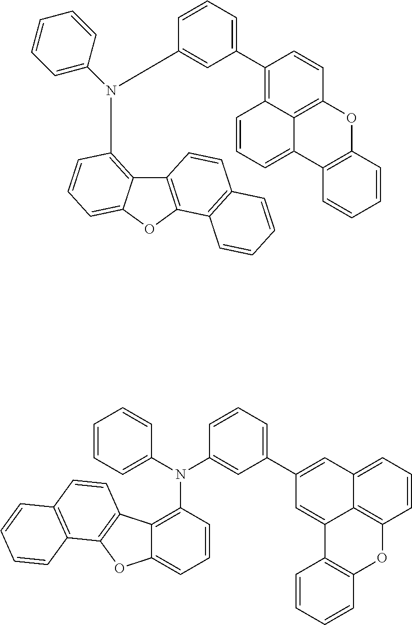 [SMILES: c1ccc(N(c2cccc(-c3ccc4c5c(cccc35)-c3ccccc3O4)c2)c2cccc3oc4c5ccccc5ccc4c23)cc1 ; c1ccc(N(c2cccc(-c3cc4c5c(cccc5c3)Oc3ccccc3-4)c2)c2cccc3oc4c5ccccc5ccc4c23)cc1]

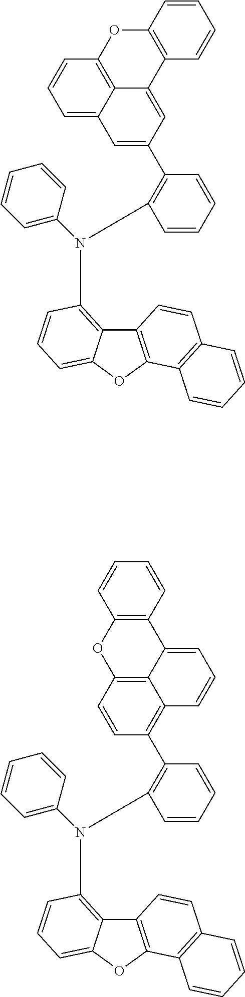 [SMILES: c1ccc(N(c2ccccc2-c2cc3c4c(cccc4c2)Oc2ccccc2-3)c2cccc3oc4c5ccccc5ccc4c23)cc1 ; c1ccc(N(c2ccccc2-c2ccc3c4c(cccc24)-c2ccccc2O3)c2cccc3oc4c5ccccc5ccc4c23)cc1]

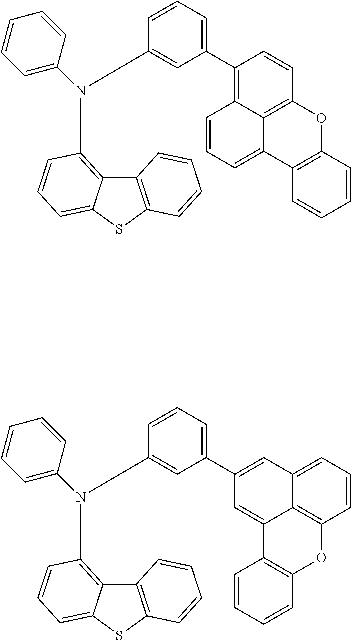 [SMILES: c1ccc(N(c2cccc(-c3ccc4c5c(cccc35)-c3ccccc3O4)c2)c2cccc3sc4ccccc4c23)cc1 ; c1ccc(N(c2cccc(-c3cc4c5c(cccc5c3)Oc3ccccc3-4)c2)c2cccc3sc4ccccc4c23)cc1]

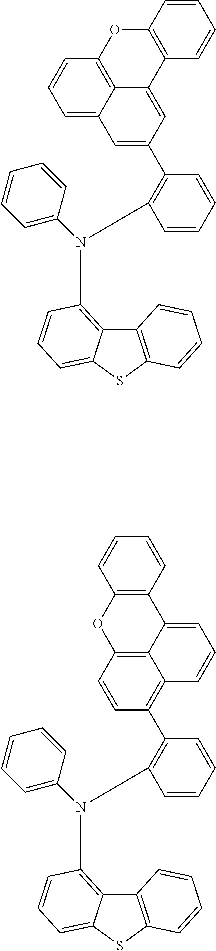 [SMILES: c1ccc(N(c2ccccc2-c2cc3c4c(cccc4c2)Oc2ccccc2-3)c2cccc3sc4ccccc4c23)cc1 ; c1ccc(N(c2ccccc2-c2ccc3c4c(cccc24)-c2ccccc2O3)c2cccc3sc4ccccc4c23)cc1]

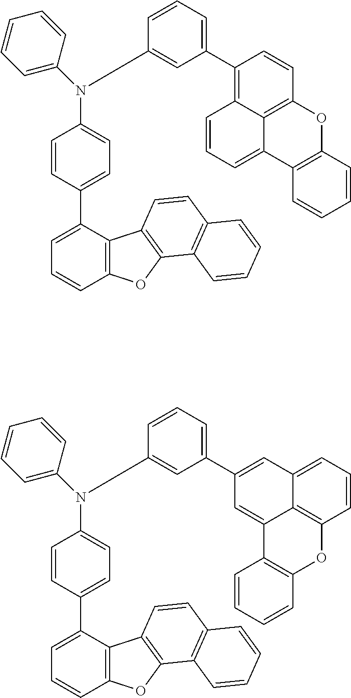 [SMILES: c1ccc(N(c2ccc(-c3cccc4oc5c6ccccc6ccc5c34)cc2)c2cccc(-c3ccc4c5c(cccc35)-c3ccccc3O4)c2)cc1 ; c1ccc(N(c2ccc(-c3cccc4oc5c6ccccc6ccc5c34)cc2)c2cccc(-c3cc4c5c(cccc5c3)Oc3ccccc3-4)c2)cc1]

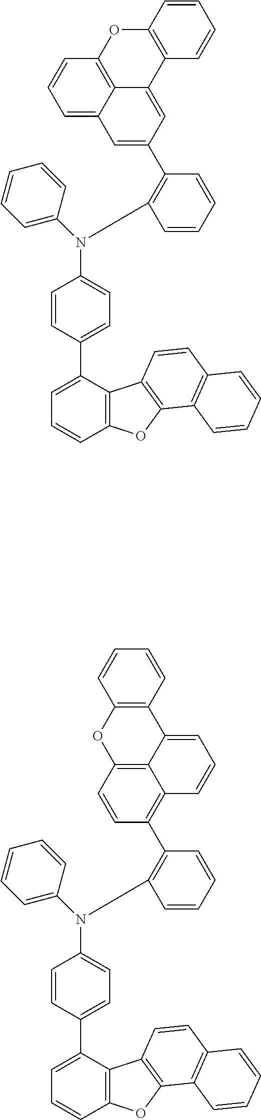 [SMILES: c1ccc(N(c2ccc(-c3cccc4oc5c6ccccc6ccc5c34)cc2)c2ccccc2-c2cc3c4c(cccc4c2)Oc2ccccc2-3)cc1 ; c1ccc(N(c2ccc(-c3cccc4oc5c6ccccc6ccc5c34)cc2)c2ccccc2-c2ccc3c4c(cccc24)-c2ccccc2O3)cc1]

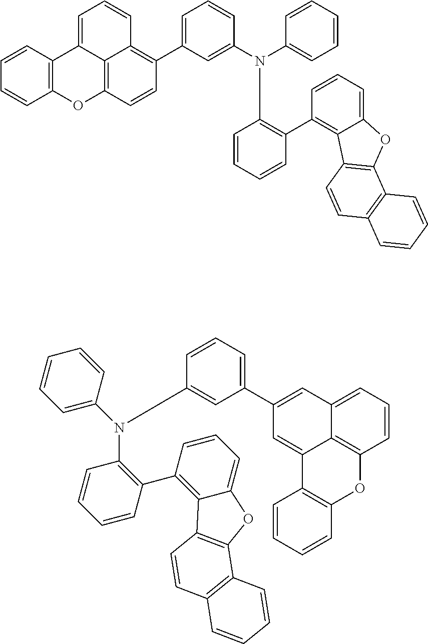 [SMILES: c1ccc(N(c2cccc(-c3ccc4c5c(cccc35)-c3ccccc3O4)c2)c2ccccc2-c2cccc3oc4c5ccccc5ccc4c23)cc1 ; c1ccc(N(c2cccc(-c3cc4c5c(cccc5c3)Oc3ccccc3-4)c2)c2ccccc2-c2cccc3oc4c5ccccc5ccc4c23)cc1]

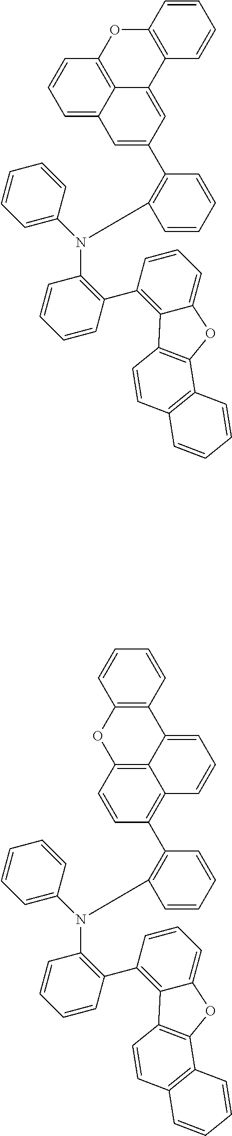 [SMILES: c1ccc(N(c2ccccc2-c2cc3c4c(cccc4c2)Oc2ccccc2-3)c2ccccc2-c2cccc3oc4c5ccccc5ccc4c23)cc1 ; c1ccc(N(c2ccccc2-c2ccc3c4c(cccc24)-c2ccccc2O3)c2ccccc2-c2cccc3oc4c5ccccc5ccc4c23)cc1]

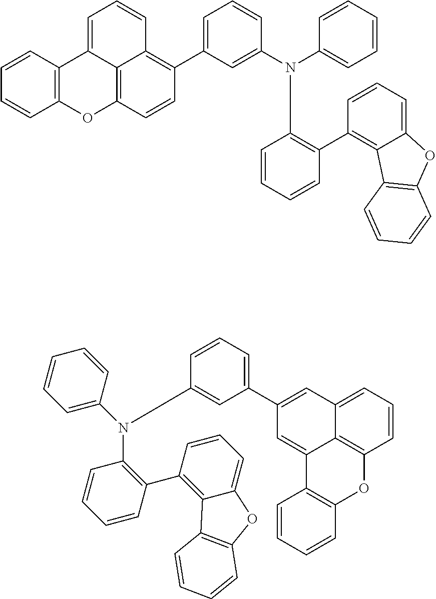 [SMILES: c1ccc(N(c2cccc(-c3ccc4c5c(cccc35)-c3ccccc3O4)c2)c2ccccc2-c2cccc3oc4ccccc4c23)cc1 ; c1ccc(N(c2cccc(-c3cc4c5c(cccc5c3)Oc3ccccc3-4)c2)c2ccccc2-c2cccc3oc4ccccc4c23)cc1]

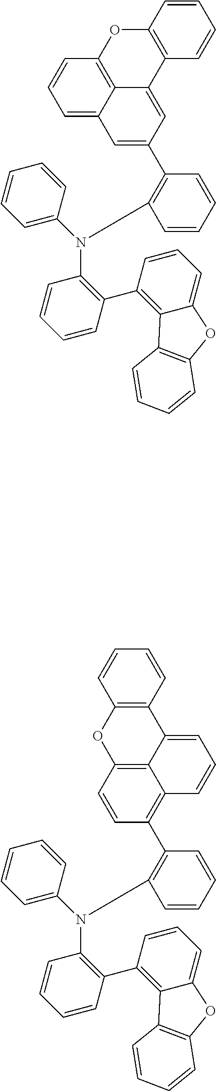 [SMILES: c1ccc(N(c2ccccc2-c2cc3c4c(cccc4c2)Oc2ccccc2-3)c2ccccc2-c2cccc3oc4ccccc4c23)cc1 ; c1ccc(N(c2ccccc2-c2ccc3c4c(cccc24)-c2ccccc2O3)c2ccccc2-c2cccc3oc4ccccc4c23)cc1]

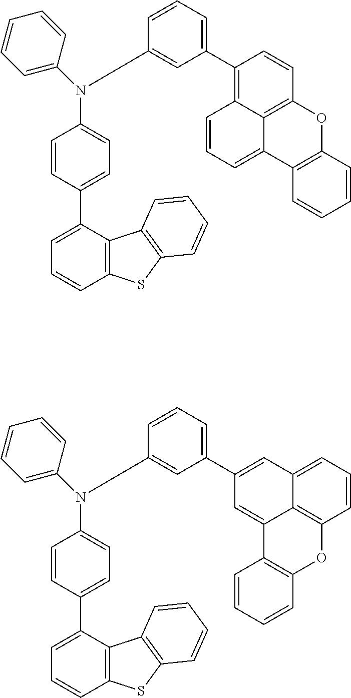 [SMILES: c1ccc(N(c2ccc(-c3cccc4sc5ccccc5c34)cc2)c2cccc(-c3ccc4c5c(cccc35)-c3ccccc3O4)c2)cc1 ; c1ccc(N(c2ccc(-c3cccc4sc5ccccc5c34)cc2)c2cccc(-c3cc4c5c(cccc5c3)Oc3ccccc3-4)c2)cc1]

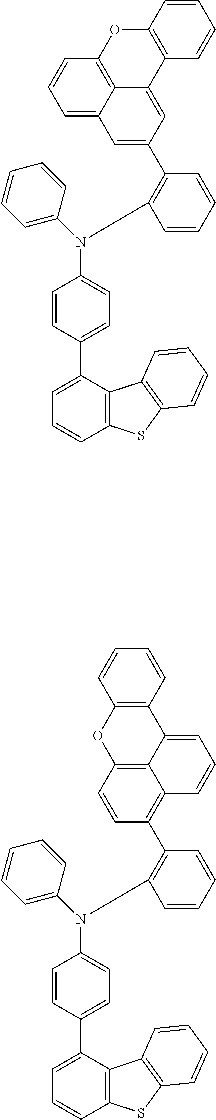 [SMILES: c1ccc(N(c2ccc(-c3cccc4sc5ccccc5c34)cc2)c2ccccc2-c2cc3c4c(cccc4c2)Oc2ccccc2-3)cc1 ; c1ccc(N(c2ccc(-c3cccc4sc5ccccc5c34)cc2)c2ccccc2-c2ccc3c4c(cccc24)-c2ccccc2O3)cc1]

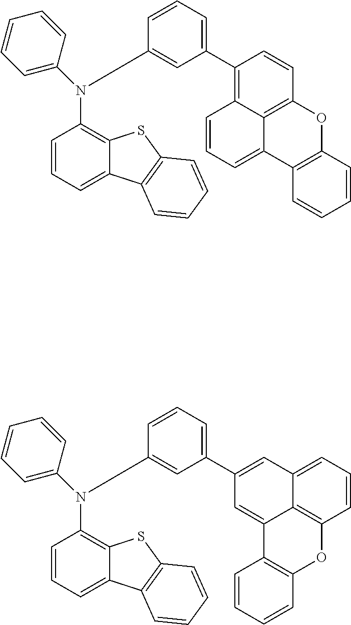 [SMILES: c1ccc(N(c2cccc(-c3ccc4c5c(cccc35)-c3ccccc3O4)c2)c2cccc3c2sc2ccccc23)cc1 ; c1ccc(N(c2cccc(-c3cc4c5c(cccc5c3)Oc3ccccc3-4)c2)c2cccc3c2sc2ccccc23)cc1]

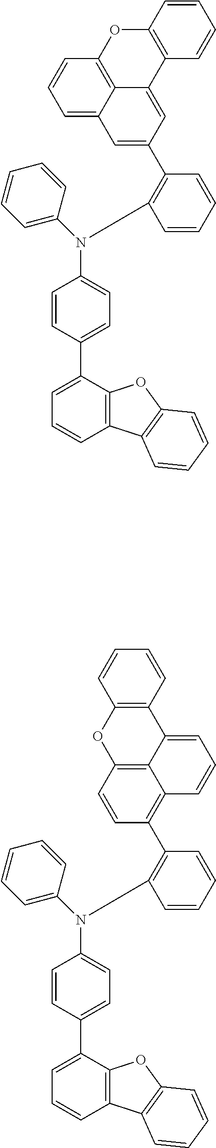 [SMILES: c1ccc(N(c2ccc(-c3cccc4c3oc3ccccc34)cc2)c2ccccc2-c2cc3c4c(cccc4c2)Oc2ccccc2-3)cc1 ; c1ccc(N(c2ccc(-c3cccc4c3oc3ccccc34)cc2)c2ccccc2-c2ccc3c4c(cccc24)-c2ccccc2O3)cc1]

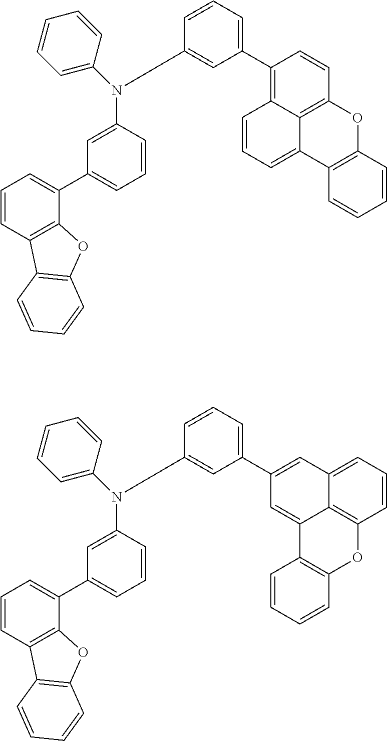 [SMILES: c1ccc(N(c2cccc(-c3ccc4c5c(cccc35)-c3ccccc3O4)c2)c2cccc(-c3cccc4c3oc3ccccc34)c2)cc1 ; c1ccc(N(c2cccc(-c3cc4c5c(cccc5c3)Oc3ccccc3-4)c2)c2cccc(-c3cccc4c3oc3ccccc34)c2)cc1]

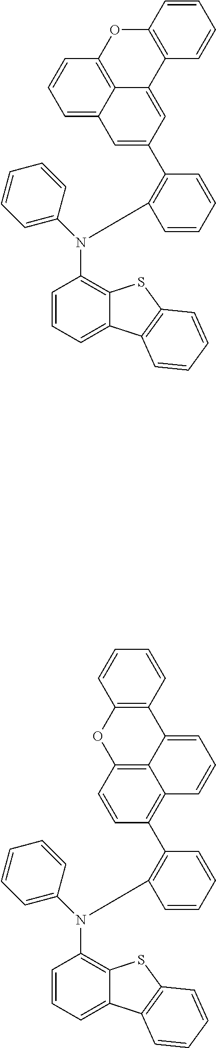 [SMILES: c1ccc(N(c2ccccc2-c2cc3c4c(cccc4c2)Oc2ccccc2-3)c2cccc3c2sc2ccccc23)cc1 ; c1ccc(N(c2ccccc2-c2ccc3c4c(cccc24)-c2ccccc2O3)c2cccc3c2sc2ccccc23)cc1]

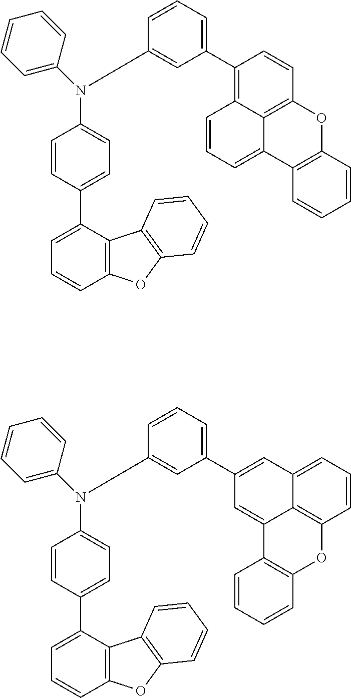 [SMILES: c1ccc(N(c2ccc(-c3cccc4oc5ccccc5c34)cc2)c2cccc(-c3ccc4c5c(cccc35)-c3ccccc3O4)c2)cc1 ; c1ccc(N(c2ccc(-c3cccc4oc5ccccc5c34)cc2)c2cccc(-c3cc4c5c(cccc5c3)Oc3ccccc3-4)c2)cc1]

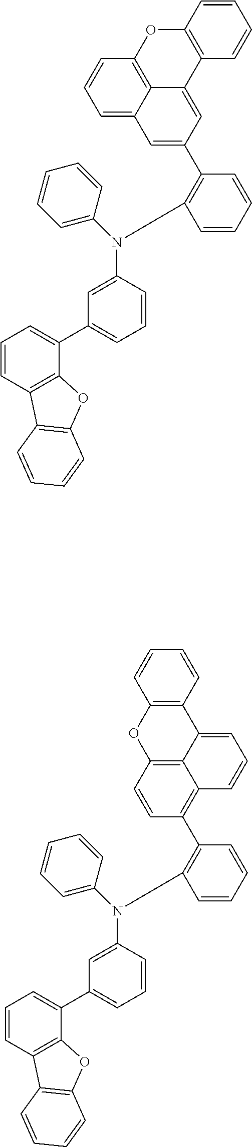 [SMILES: c1ccc(N(c2cccc(-c3cccc4c3oc3ccccc34)c2)c2ccccc2-c2cc3c4c(cccc4c2)Oc2ccccc2-3)cc1 ; c1ccc(N(c2cccc(-c3cccc4c3oc3ccccc34)c2)c2ccccc2-c2ccc3c4c(cccc24)-c2ccccc2O3)cc1]

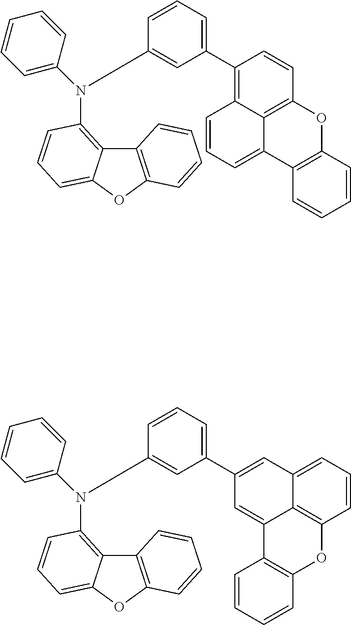 [SMILES: c1ccc(N(c2cccc(-c3ccc4c5c(cccc35)-c3ccccc3O4)c2)c2cccc3oc4ccccc4c23)cc1 ; c1ccc(N(c2cccc(-c3cc4c5c(cccc5c3)Oc3ccccc3-4)c2)c2cccc3oc4ccccc4c23)cc1]

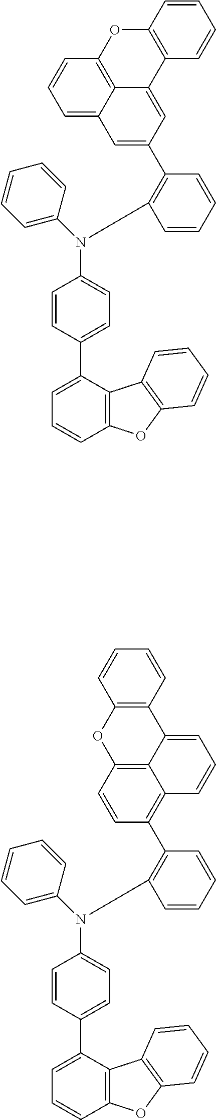 [SMILES: c1ccc(N(c2ccc(-c3cccc4oc5ccccc5c34)cc2)c2ccccc2-c2cc3c4c(cccc4c2)Oc2ccccc2-3)cc1 ; c1ccc(N(c2ccc(-c3cccc4oc5ccccc5c34)cc2)c2ccccc2-c2ccc3c4c(cccc24)-c2ccccc2O3)cc1]

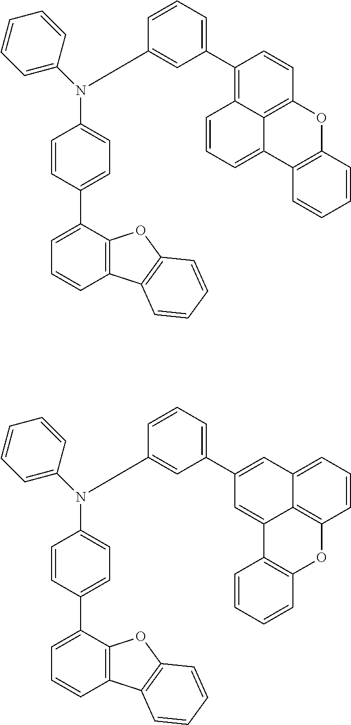 [SMILES: c1ccc(N(c2ccc(-c3cccc4c3oc3ccccc34)cc2)c2cccc(-c3ccc4c5c(cccc35)-c3ccccc3O4)c2)cc1 ; c1ccc(N(c2ccc(-c3cccc4c3oc3ccccc34)cc2)c2cccc(-c3cc4c5c(cccc5c3)Oc3ccccc3-4)c2)cc1]

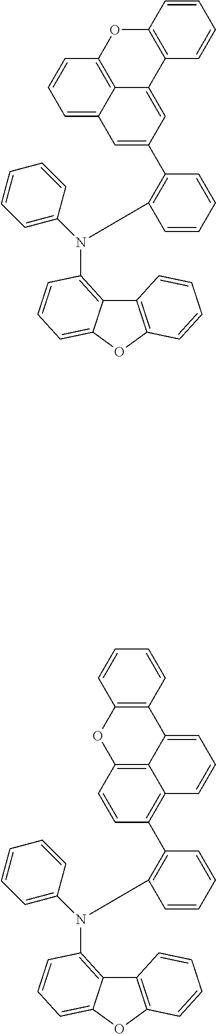 [SMILES: c1ccc(N(c2ccccc2-c2cc3c4c(cccc4c2)Oc2ccccc2-3)c2cccc3oc4ccccc4c23)cc1 ; c1ccc(N(c2ccccc2-c2ccc3c4c(cccc24)-c2ccccc2O3)c2cccc3oc4ccccc4c23)cc1]

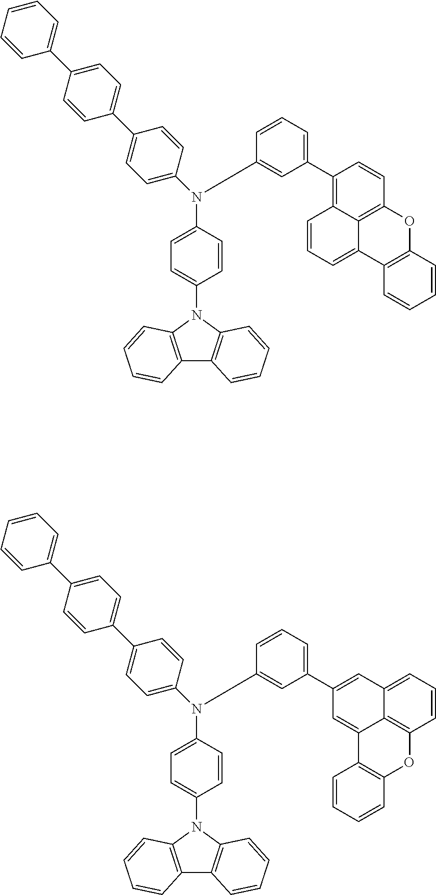 [SMILES: c1ccc(-c2ccc(-c3ccc(N(c4ccc(-n5c6ccccc6c6ccccc65)cc4)c4cccc(-c5ccc6c7c(cccc57)-c5ccccc5O6)c4)cc3)cc2)cc1 ; c1ccc(-c2ccc(-c3ccc(N(c4ccc(-n5c6ccccc6c6ccccc65)cc4)c4cccc(-c5cc6c7c(cccc7c5)Oc5ccccc5-6)c4)cc3)cc2)cc1]

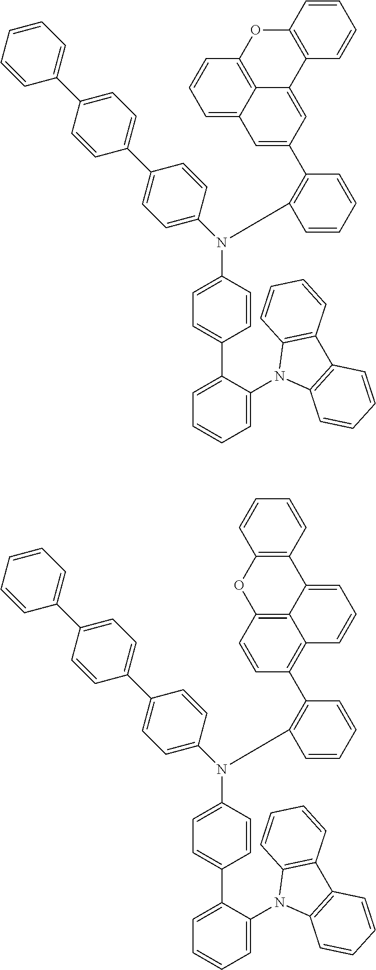 [SMILES: c1ccc(-c2ccc(-c3ccc(N(c4ccc(-c5ccccc5-n5c6ccccc6c6ccccc65)cc4)c4ccccc4-c4cc5c6c(cccc6c4)Oc4ccccc4-5)cc3)cc2)cc1 ; c1ccc(-c2ccc(-c3ccc(N(c4ccc(-c5ccccc5-n5c6ccccc6c6ccccc65)cc4)c4ccccc4-c4ccc5c6c(cccc46)-c4ccccc4O5)cc3)cc2)cc1]

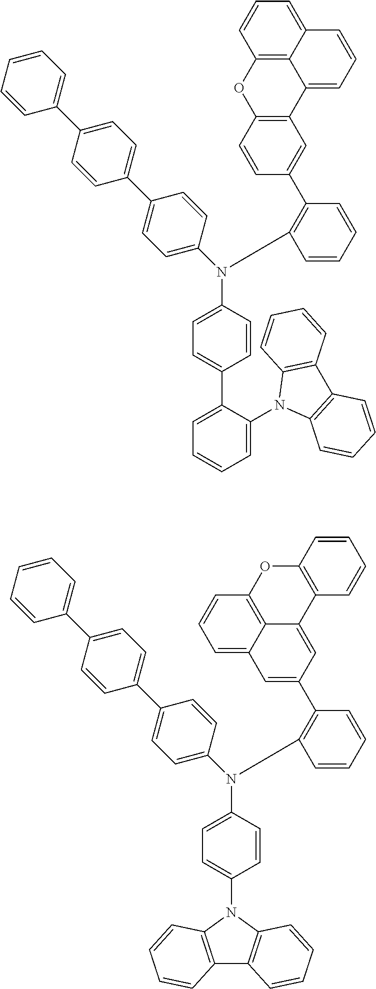 [SMILES: c1ccc(-c2ccc(-c3ccc(N(c4ccc(-c5ccccc5-n5c6ccccc6c6ccccc65)cc4)c4ccccc4-c4ccc5c(c4)-c4cccc6cccc(c46)O5)cc3)cc2)cc1 ; c1ccc(-c2ccc(-c3ccc(N(c4ccc(-n5c6ccccc6c6ccccc65)cc4)c4ccccc4-c4cc5c6c(cccc6c4)Oc4ccccc4-5)cc3)cc2)cc1]

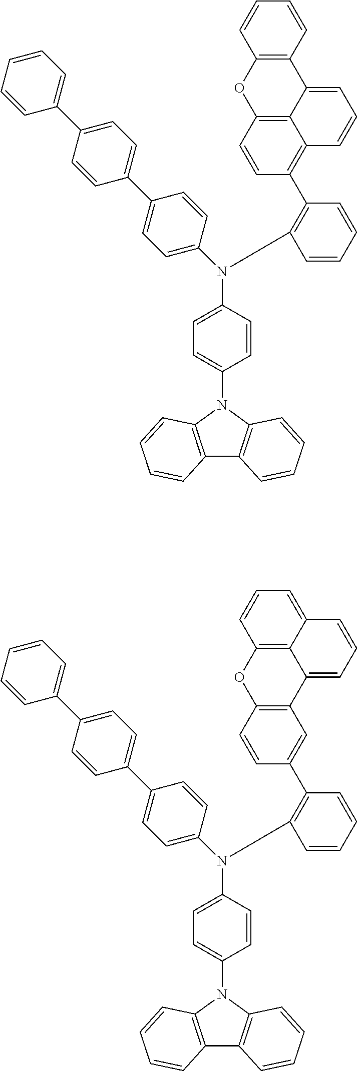 [SMILES: c1ccc(-c2ccc(-c3ccc(N(c4ccc(-n5c6ccccc6c6ccccc65)cc4)c4ccccc4-c4ccc5c6c(cccc46)-c4ccccc4O5)cc3)cc2)cc1 ; c1ccc(-c2ccc(-c3ccc(N(c4ccc(-n5c6ccccc6c6ccccc65)cc4)c4ccccc4-c4ccc5c(c4)-c4cccc6cccc(c46)O5)cc3)cc2)cc1]

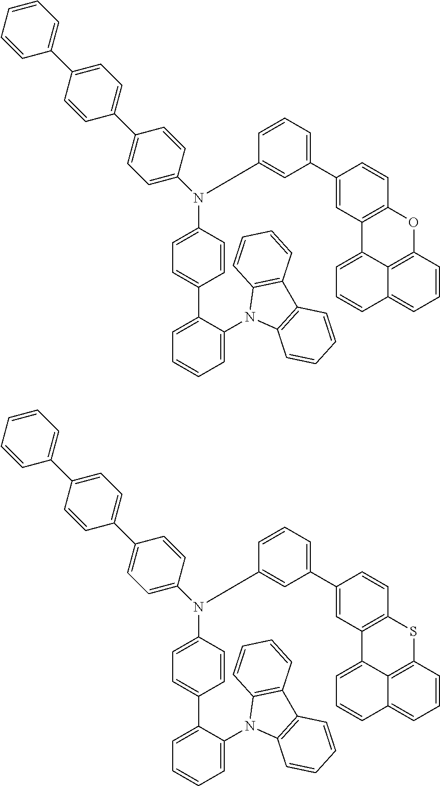 [SMILES: c1ccc(-c2ccc(-c3ccc(N(c4ccc(-c5ccccc5-n5c6ccccc6c6ccccc65)cc4)c4cccc(-c5ccc6c(c5)-c5cccc7cccc(c57)O6)c4)cc3)cc2)cc1 ; c1ccc(-c2ccc(-c3ccc(N(c4ccc(-c5ccccc5-n5c6ccccc6c6ccccc65)cc4)c4cccc(-c5ccc6c(c5)-c5cccc7cccc(c57)S6)c4)cc3)cc2)cc1]

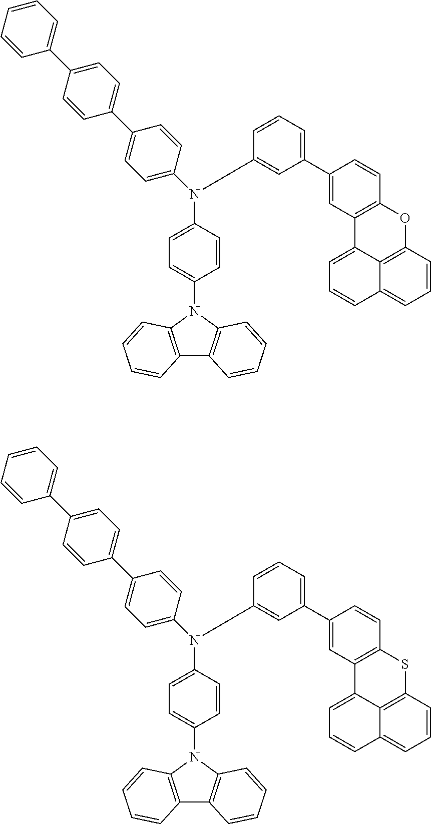 [SMILES: c1ccc(-c2ccc(-c3ccc(N(c4ccc(-n5c6ccccc6c6ccccc65)cc4)c4cccc(-c5ccc6c(c5)-c5cccc7cccc(c57)O6)c4)cc3)cc2)cc1 ; c1ccc(-c2ccc(-c3ccc(N(c4ccc(-n5c6ccccc6c6ccccc65)cc4)c4cccc(-c5ccc6c(c5)-c5cccc7cccc(c57)S6)c4)cc3)cc2)cc1]

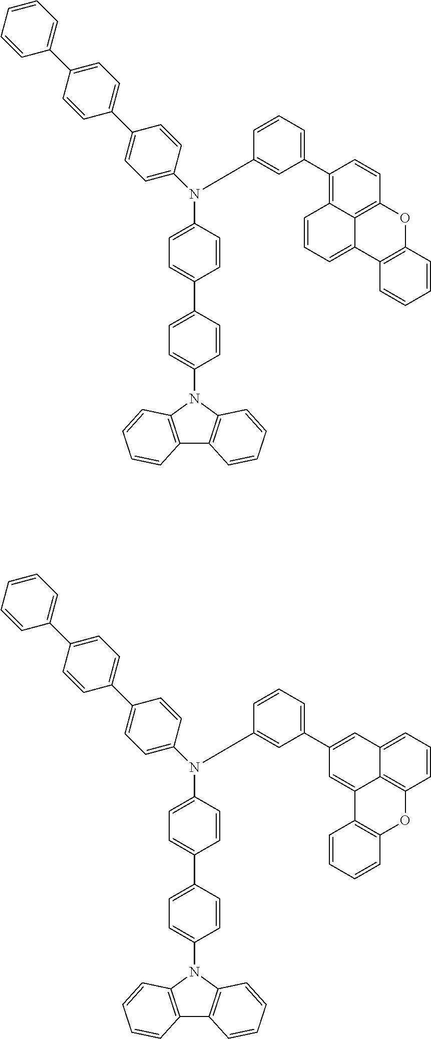 [SMILES: c1ccc(-c2ccc(-c3ccc(N(c4ccc(-c5ccc(-n6c7ccccc7c7ccccc76)cc5)cc4)c4cccc(-c5ccc6c7c(cccc57)-c5ccccc5O6)c4)cc3)cc2)cc1 ; c1ccc(-c2ccc(-c3ccc(N(c4ccc(-c5ccc(-n6c7ccccc7c7ccccc76)cc5)cc4)c4cccc(-c5cc6c7c(cccc7c5)Oc5ccccc5-6)c4)cc3)cc2)cc1]

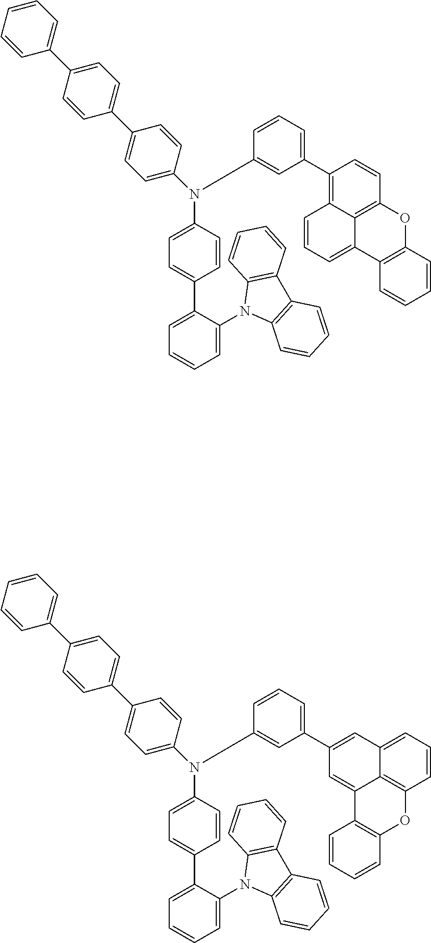 [SMILES: c1ccc(-c2ccc(-c3ccc(N(c4ccc(-c5ccccc5-n5c6ccccc6c6ccccc65)cc4)c4cccc(-c5ccc6c7c(cccc57)-c5ccccc5O6)c4)cc3)cc2)cc1 ; c1ccc(-c2ccc(-c3ccc(N(c4ccc(-c5ccccc5-n5c6ccccc6c6ccccc65)cc4)c4cccc(-c5cc6c7c(cccc7c5)Oc5ccccc5-6)c4)cc3)cc2)cc1]

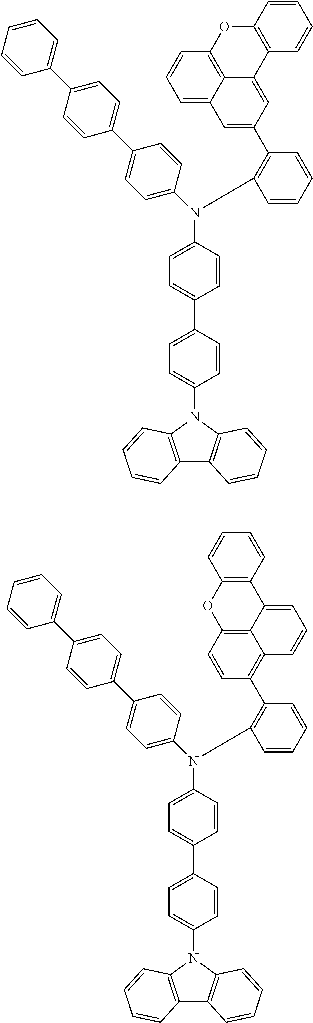 [SMILES: c1ccc(-c2ccc(-c3ccc(N(c4ccc(-c5ccc(-n6c7ccccc7c7ccccc76)cc5)cc4)c4ccccc4-c4cc5c6c(cccc6c4)Oc4ccccc4-5)cc3)cc2)cc1 ; c1ccc(-c2ccc(-c3ccc(N(c4ccc(-c5ccc(-n6c7ccccc7c7ccccc76)cc5)cc4)c4ccccc4-c4ccc5c6c(cccc46)-c4ccccc4O5)cc3)cc2)cc1]

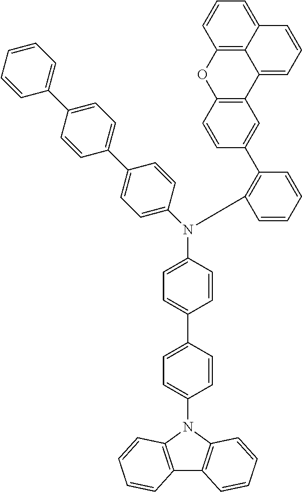 [SMILES: c1ccc(-c2ccc(-c3ccc(N(c4ccc(-c5ccc(-n6c7ccccc7c7ccccc76)cc5)cc4)c4ccccc4-c4ccc5c(c4)-c4cccc6cccc(c46)O5)cc3)cc2)cc1]

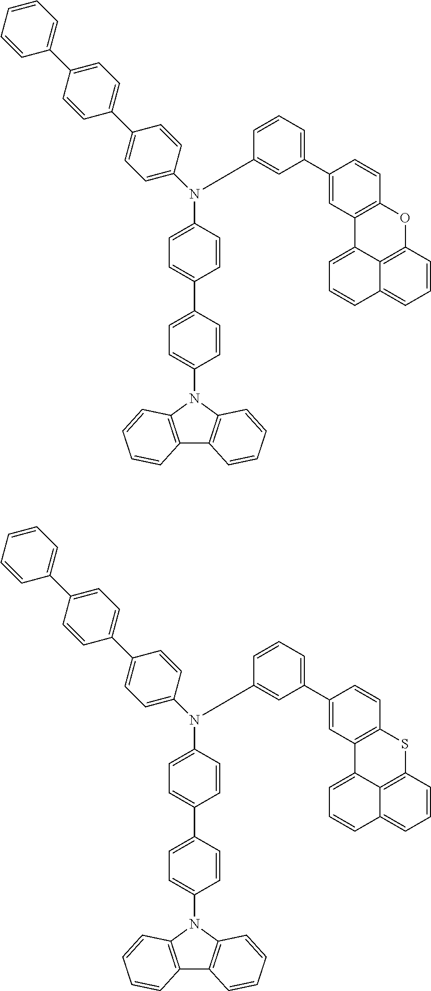 [SMILES: c1ccc(-c2ccc(-c3ccc(N(c4ccc(-c5ccc(-n6c7ccccc7c7ccccc76)cc5)cc4)c4cccc(-c5ccc6c(c5)-c5cccc7cccc(c57)O6)c4)cc3)cc2)cc1 ; c1ccc(-c2ccc(-c3ccc(N(c4ccc(-c5ccc(-n6c7ccccc7c7ccccc76)cc5)cc4)c4cccc(-c5ccc6c(c5)-c5cccc7cccc(c57)S6)c4)cc3)cc2)cc1]

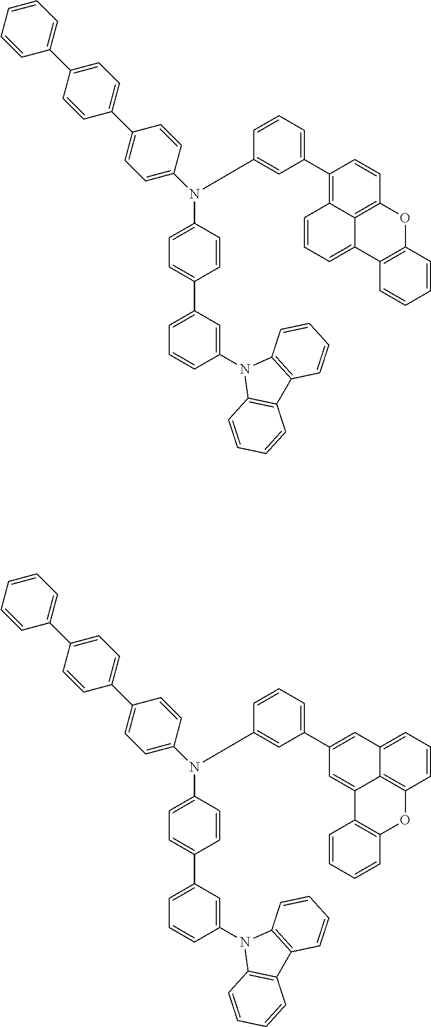 [SMILES: c1ccc(-c2ccc(-c3ccc(N(c4ccc(-c5cccc(-n6c7ccccc7c7ccccc76)c5)cc4)c4cccc(-c5ccc6c7c(cccc57)-c5ccccc5O6)c4)cc3)cc2)cc1 ; c1ccc(-c2ccc(-c3ccc(N(c4ccc(-c5cccc(-n6c7ccccc7c7ccccc76)c5)cc4)c4cccc(-c5cc6c7c(cccc7c5)Oc5ccccc5-6)c4)cc3)cc2)cc1]

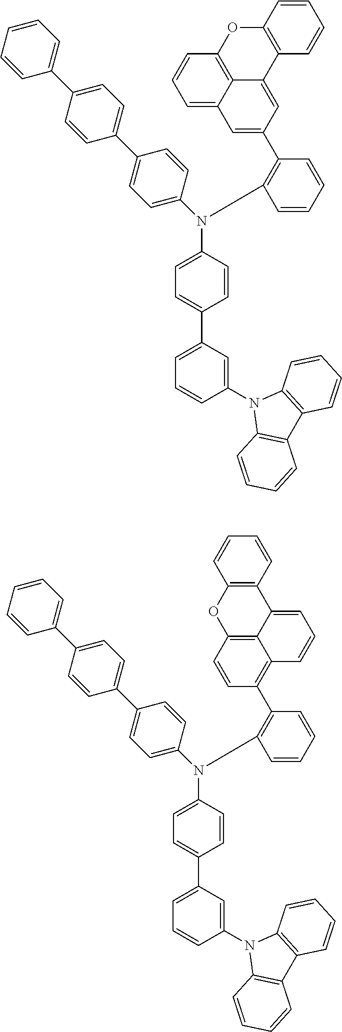 [SMILES: c1ccc(-c2ccc(-c3ccc(N(c4ccc(-c5cccc(-n6c7ccccc7c7ccccc76)c5)cc4)c4ccccc4-c4cc5c6c(cccc6c4)Oc4ccccc4-5)cc3)cc2)cc1 ; c1ccc(-c2ccc(-c3ccc(N(c4ccc(-c5cccc(-n6c7ccccc7c7ccccc76)c5)cc4)c4ccccc4-c4ccc5c6c(cccc46)-c4ccccc4O5)cc3)cc2)cc1]

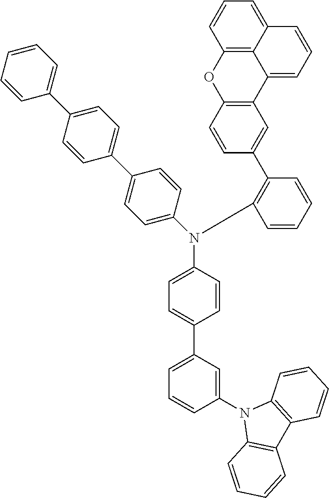 [SMILES: c1ccc(-c2ccc(-c3ccc(N(c4ccc(-c5cccc(-n6c7ccccc7c7ccccc76)c5)cc4)c4ccccc4-c4ccc5c(c4)-c4cccc6cccc(c46)O5)cc3)cc2)cc1]

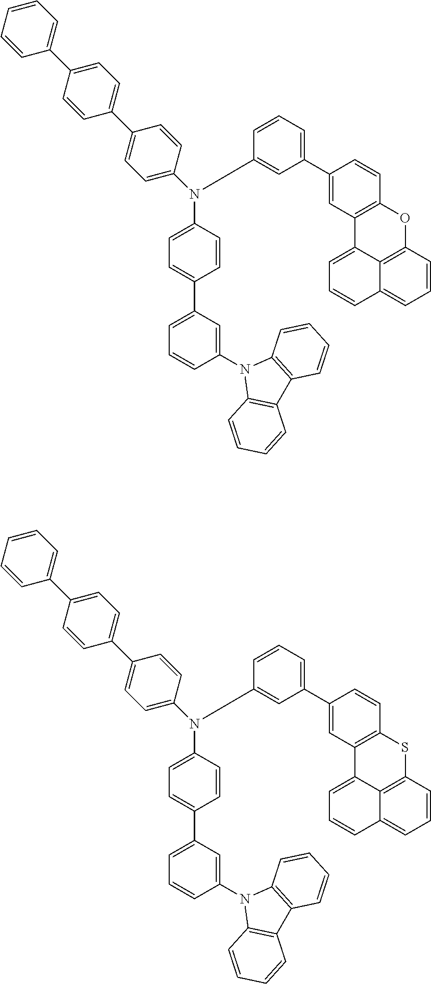 [SMILES: c1ccc(-c2ccc(-c3ccc(N(c4ccc(-c5cccc(-n6c7ccccc7c7ccccc76)c5)cc4)c4cccc(-c5ccc6c(c5)-c5cccc7cccc(c57)O6)c4)cc3)cc2)cc1 ; c1ccc(-c2ccc(-c3ccc(N(c4ccc(-c5cccc(-n6c7ccccc7c7ccccc76)c5)cc4)c4cccc(-c5ccc6c(c5)-c5cccc7cccc(c57)S6)c4)cc3)cc2)cc1]

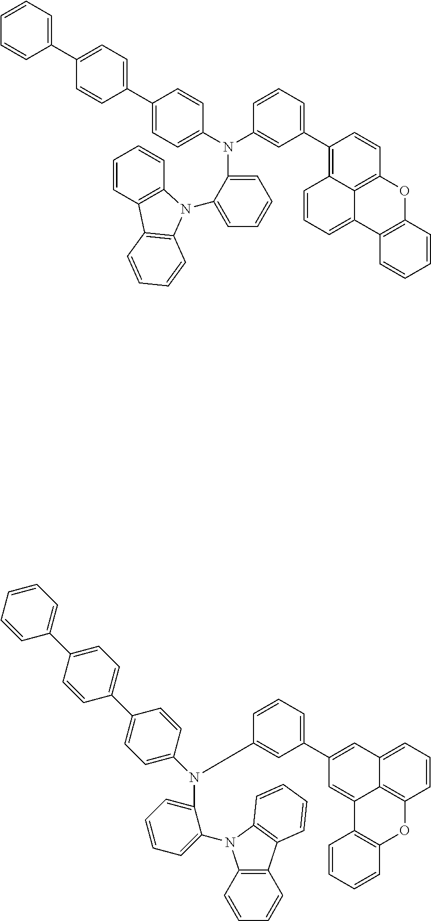 [SMILES: c1ccc(-c2ccc(-c3ccc(N(c4cccc(-c5ccc6c7c(cccc57)-c5ccccc5O6)c4)c4ccccc4-n4c5ccccc5c5ccccc54)cc3)cc2)cc1 ; c1ccc(-c2ccc(-c3ccc(N(c4cccc(-c5cc6c7c(cccc7c5)Oc5ccccc5-6)c4)c4ccccc4-n4c5ccccc5c5ccccc54)cc3)cc2)cc1]

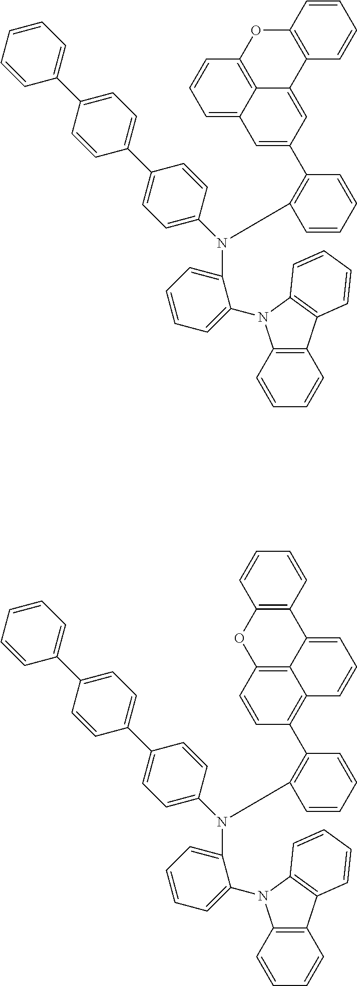 [SMILES: c1ccc(-c2ccc(-c3ccc(N(c4ccccc4-c4cc5c6c(cccc6c4)Oc4ccccc4-5)c4ccccc4-n4c5ccccc5c5ccccc54)cc3)cc2)cc1 ; c1ccc(-c2ccc(-c3ccc(N(c4ccccc4-c4ccc5c6c(cccc46)-c4ccccc4O5)c4ccccc4-n4c5ccccc5c5ccccc54)cc3)cc2)cc1]

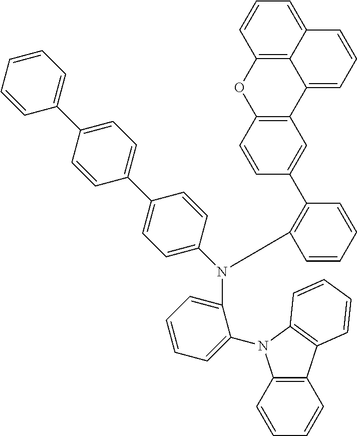 [SMILES: c1ccc(-c2ccc(-c3ccc(N(c4ccccc4-c4ccc5c(c4)-c4cccc6cccc(c46)O5)c4ccccc4-n4c5ccccc5c5ccccc54)cc3)cc2)cc1]

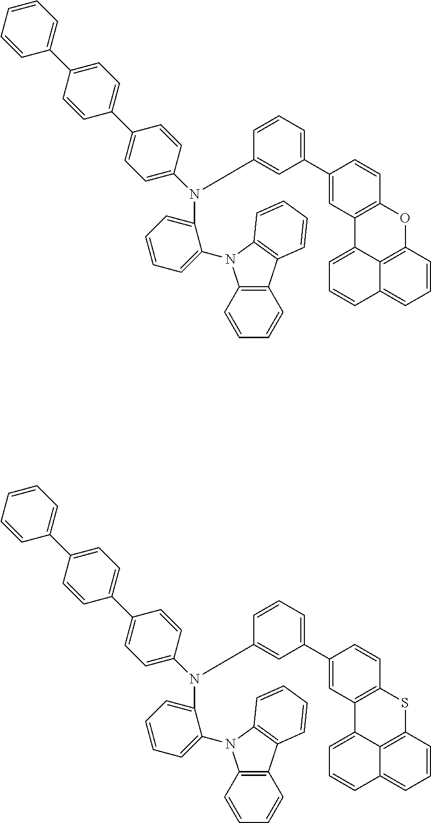 [SMILES: c1ccc(-c2ccc(-c3ccc(N(c4cccc(-c5ccc6c(c5)-c5cccc7cccc(c57)O6)c4)c4ccccc4-n4c5ccccc5c5ccccc54)cc3)cc2)cc1 ; c1ccc(-c2ccc(-c3ccc(N(c4cccc(-c5ccc6c(c5)-c5cccc7cccc(c57)S6)c4)c4ccccc4-n4c5ccccc5c5ccccc54)cc3)cc2)cc1]

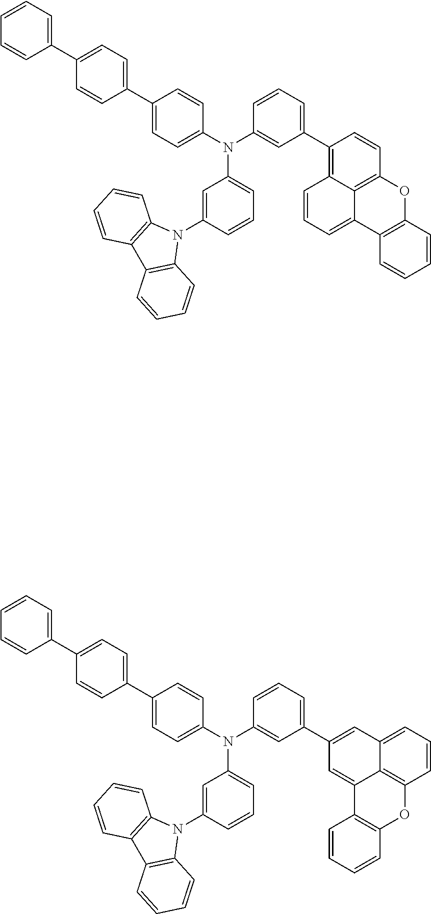 [SMILES: c1ccc(-c2ccc(-c3ccc(N(c4cccc(-c5ccc6c7c(cccc57)-c5ccccc5O6)c4)c4cccc(-n5c6ccccc6c6ccccc65)c4)cc3)cc2)cc1 ; c1ccc(-c2ccc(-c3ccc(N(c4cccc(-c5cc6c7c(cccc7c5)Oc5ccccc5-6)c4)c4cccc(-n5c6ccccc6c6ccccc65)c4)cc3)cc2)cc1]

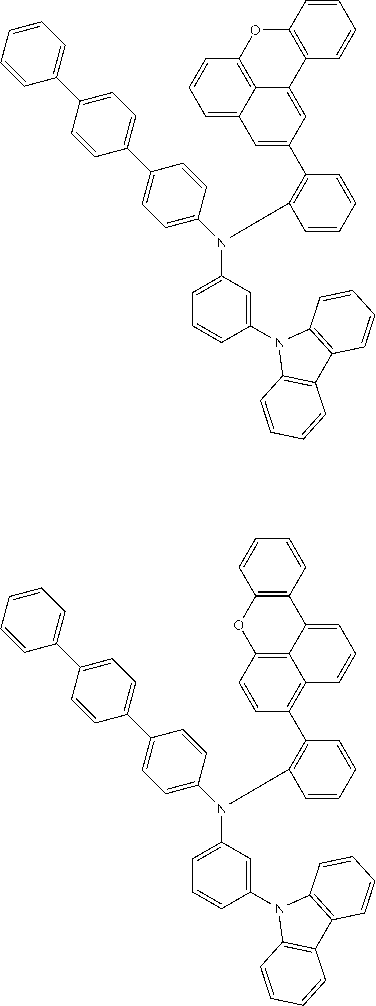 [SMILES: c1ccc(-c2ccc(-c3ccc(N(c4cccc(-n5c6ccccc6c6ccccc65)c4)c4ccccc4-c4cc5c6c(cccc6c4)Oc4ccccc4-5)cc3)cc2)cc1 ; c1ccc(-c2ccc(-c3ccc(N(c4cccc(-n5c6ccccc6c6ccccc65)c4)c4ccccc4-c4ccc5c6c(cccc46)-c4ccccc4O5)cc3)cc2)cc1]

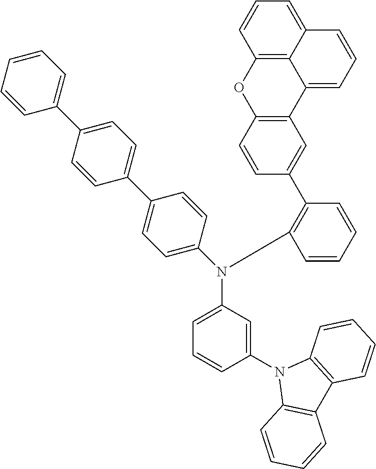 [SMILES: c1ccc(-c2ccc(-c3ccc(N(c4cccc(-n5c6ccccc6c6ccccc65)c4)c4ccccc4-c4ccc5c(c4)-c4cccc6cccc(c46)O5)cc3)cc2)cc1]

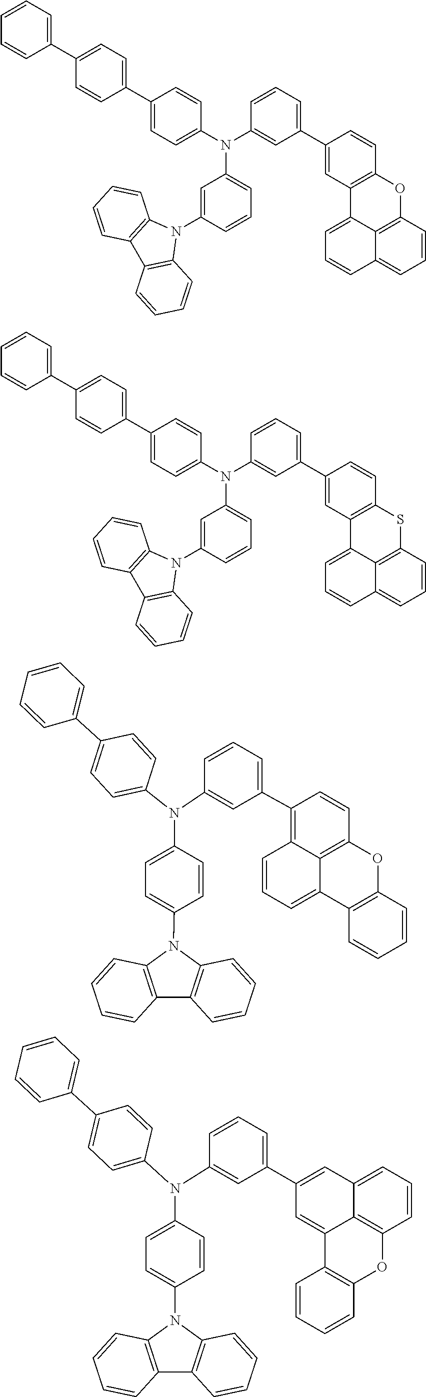 [SMILES: c1ccc(-c2ccc(-c3ccc(N(c4cccc(-c5ccc6c(c5)-c5cccc7cccc(c57)O6)c4)c4cccc(-n5c6ccccc6c6ccccc65)c4)cc3)cc2)cc1 ; c1ccc(-c2ccc(-c3ccc(N(c4cccc(-c5ccc6c(c5)-c5cccc7cccc(c57)S6)c4)c4cccc(-n5c6ccccc6c6ccccc65)c4)cc3)cc2)cc1 ; c1ccc(-c2ccc(N(c3ccc(-n4c5ccccc5c5ccccc54)cc3)c3cccc(-c4ccc5c6c(cccc46)-c4ccccc4O5)c3)cc2)cc1 ; c1ccc(-c2ccc(N(c3ccc(-n4c5ccccc5c5ccccc54)cc3)c3cccc(-c4cc5c6c(cccc6c4)Oc4ccccc4-5)c3)cc2)cc1]

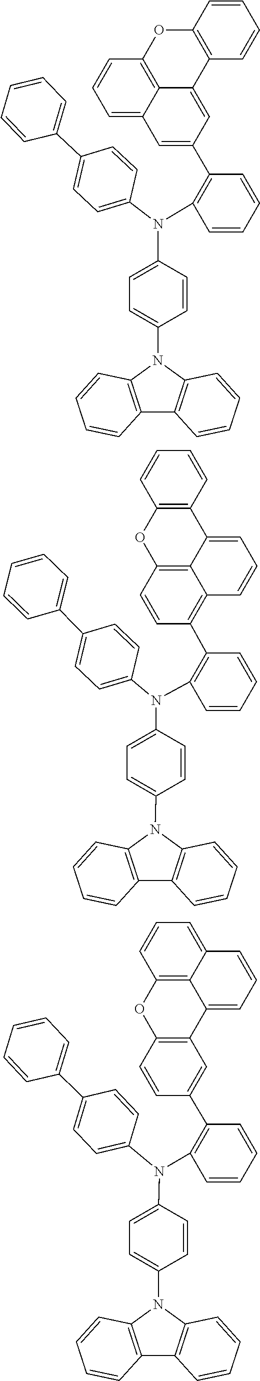

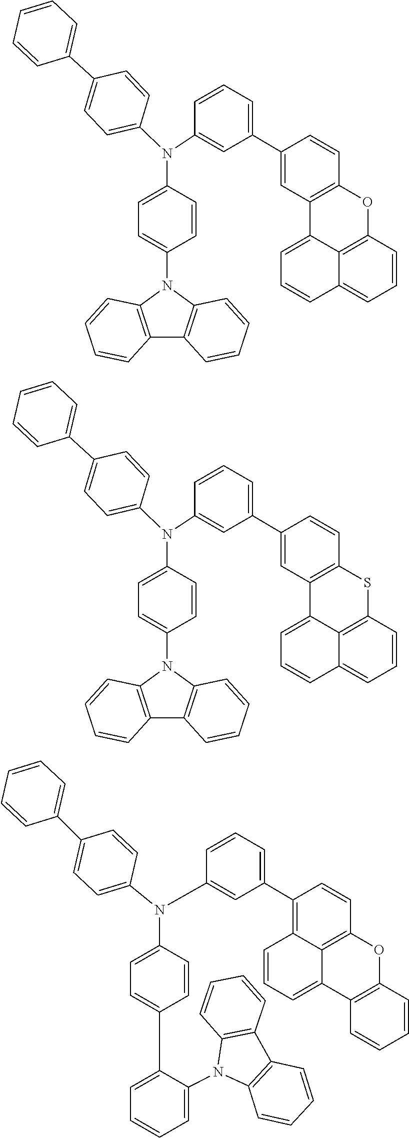 [SMILES: c1ccc(-c2ccc(N(c3ccc(-n4c5ccccc5c5ccccc54)cc3)c3cccc(-c4ccc5c(c4)-c4cccc6cccc(c46)O5)c3)cc2)cc1 ; c1ccc(-c2ccc(N(c3ccc(-n4c5ccccc5c5ccccc54)cc3)c3cccc(-c4ccc5c(c4)-c4cccc6cccc(c46)S5)c3)cc2)cc1 ; c1ccc(-c2ccc(N(c3ccc(-c4ccccc4-n4c5ccccc5c5ccccc54)cc3)c3cccc(-c4ccc5c6c(cccc46)-c4ccccc4O5)c3)cc2)cc1]

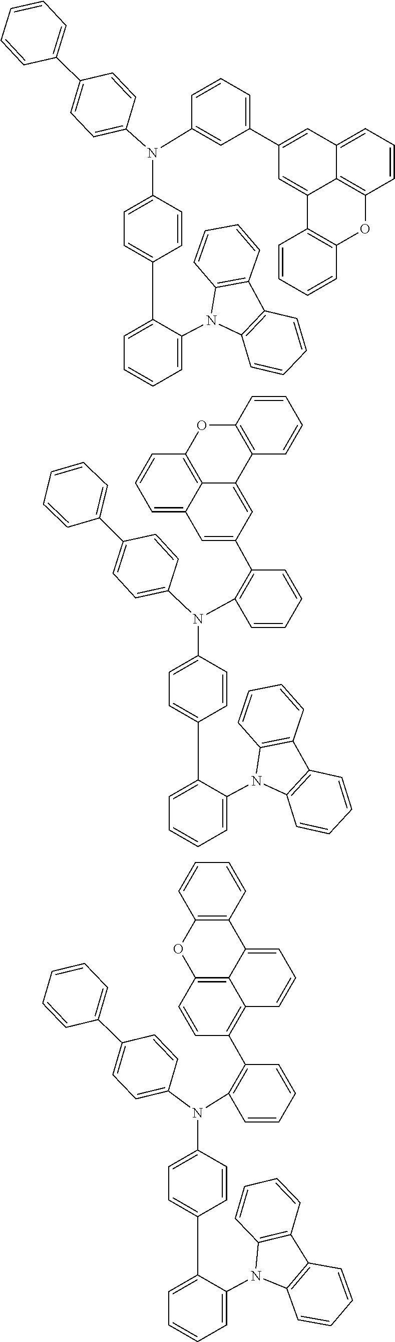

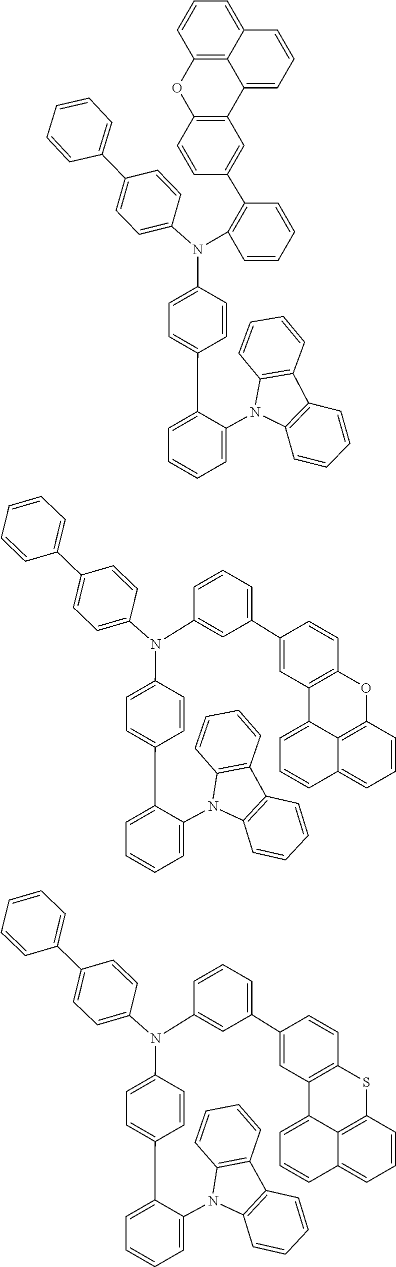 [SMILES: c1ccc(-c2ccc(N(c3ccc(-c4ccccc4-n4c5ccccc5c5ccccc54)cc3)c3ccccc3-c3ccc4c(c3)-c3cccc5cccc(c35)O4)cc2)cc1 ; c1ccc(-c2ccc(N(c3ccc(-c4ccccc4-n4c5ccccc5c5ccccc54)cc3)c3cccc(-c4ccc5c(c4)-c4cccc6cccc(c46)O5)c3)cc2)cc1 ; c1ccc(-c2ccc(N(c3ccc(-c4ccccc4-n4c5ccccc5c5ccccc54)cc3)c3cccc(-c4ccc5c(c4)-c4cccc6cccc(c46)S5)c3)cc2)cc1]

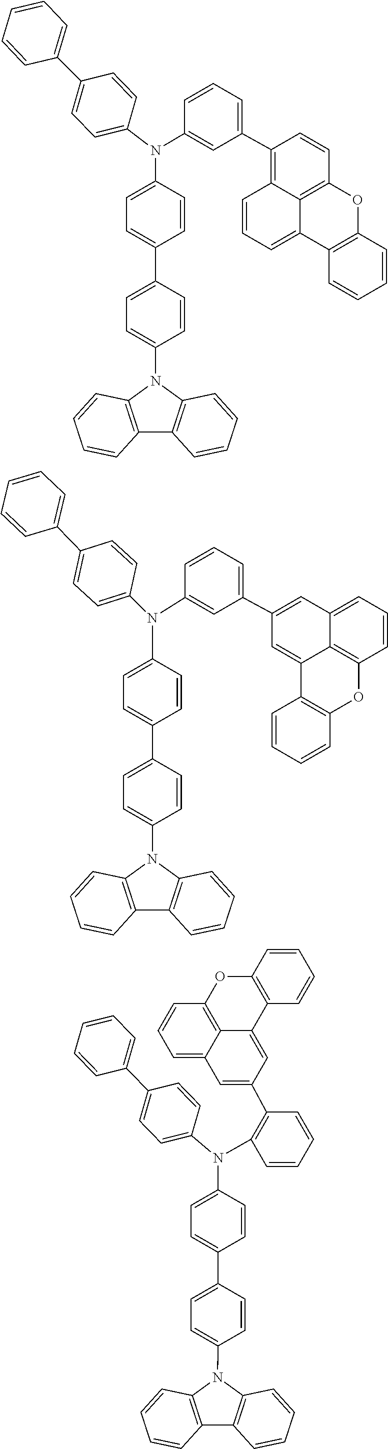 [SMILES: c1ccc(-c2ccc(N(c3ccc(-c4ccc(-n5c6ccccc6c6ccccc65)cc4)cc3)c3cccc(-c4ccc5c6c(cccc46)-c4ccccc4O5)c3)cc2)cc1 ; c1ccc(-c2ccc(N(c3ccc(-c4ccc(-n5c6ccccc6c6ccccc65)cc4)cc3)c3cccc(-c4cc5c6c(cccc6c4)Oc4ccccc4-5)c3)cc2)cc1 ; c1ccc(-c2ccc(N(c3ccc(-c4ccc(-n5c6ccccc6c6ccccc65)cc4)cc3)c3ccccc3-c3cc4c5c(cccc5c3)Oc3ccccc3-4)cc2)cc1]

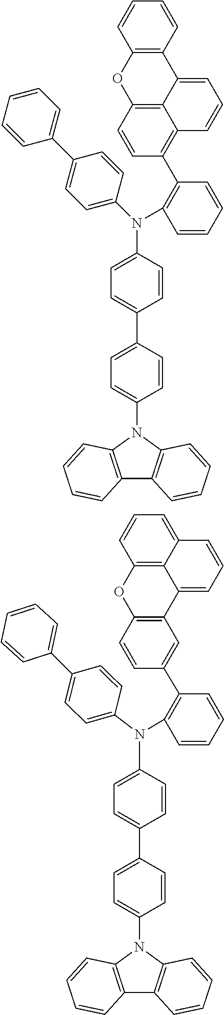

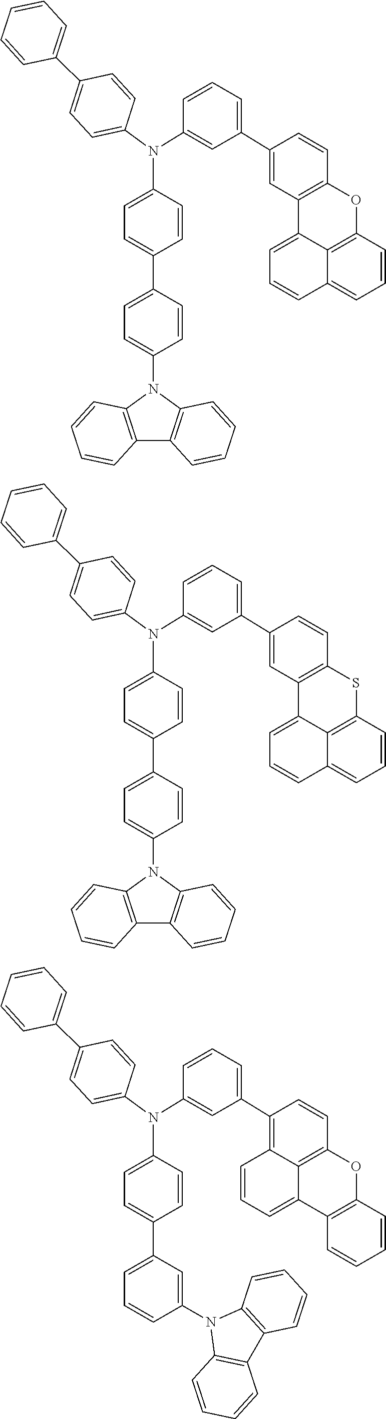

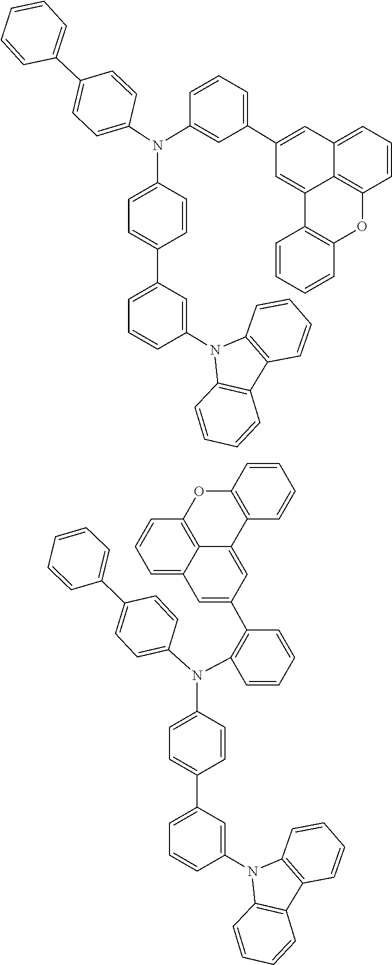

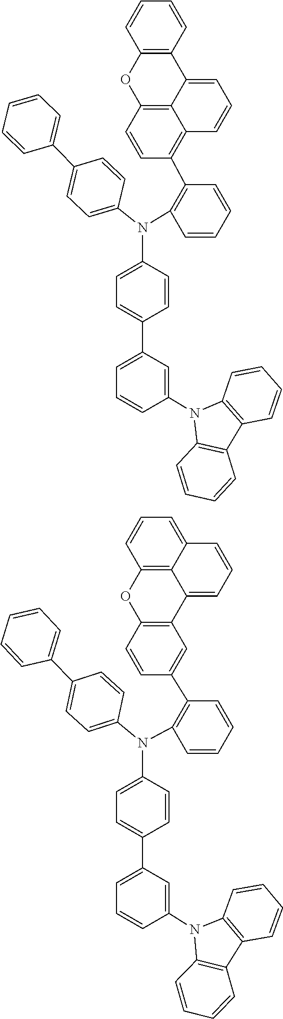

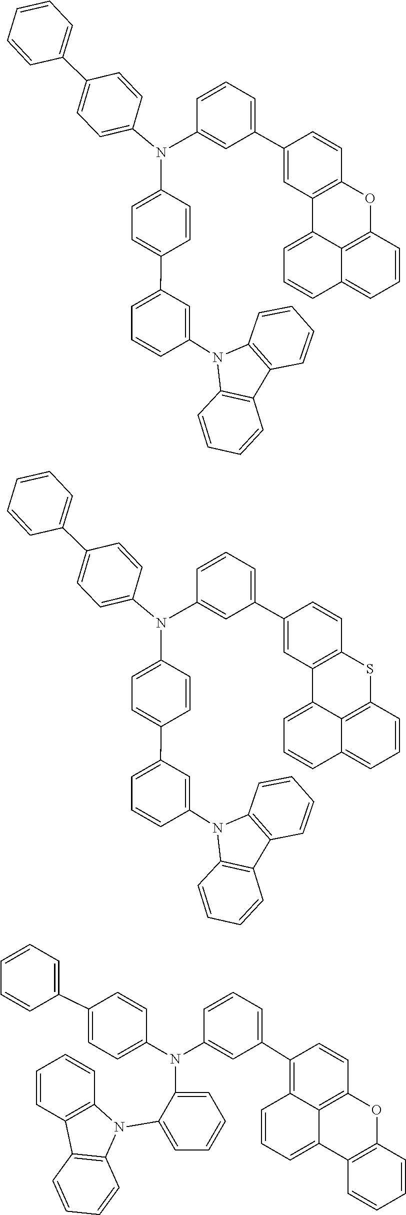 [SMILES: c1ccc(-c2ccc(N(c3ccc(-c4cccc(-n5c6ccccc6c6ccccc65)c4)cc3)c3cccc(-c4ccc5c(c4)-c4cccc6cccc(c46)O5)c3)cc2)cc1 ; c1ccc(-c2ccc(N(c3ccc(-c4cccc(-n5c6ccccc6c6ccccc65)c4)cc3)c3cccc(-c4ccc5c(c4)-c4cccc6cccc(c46)S5)c3)cc2)cc1 ; c1ccc(-c2ccc(N(c3cccc(-c4ccc5c6c(cccc46)-c4ccccc4O5)c3)c3ccccc3-n3c4ccccc4c4ccccc43)cc2)cc1]

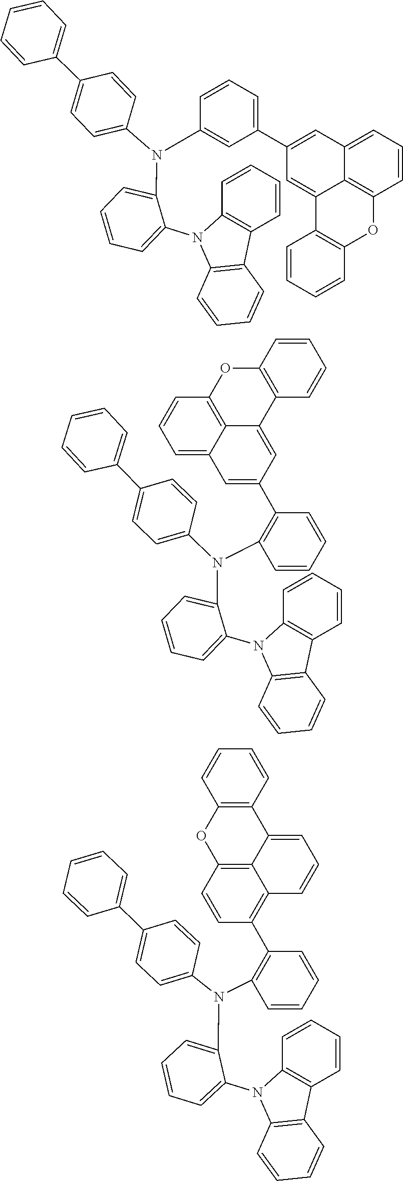

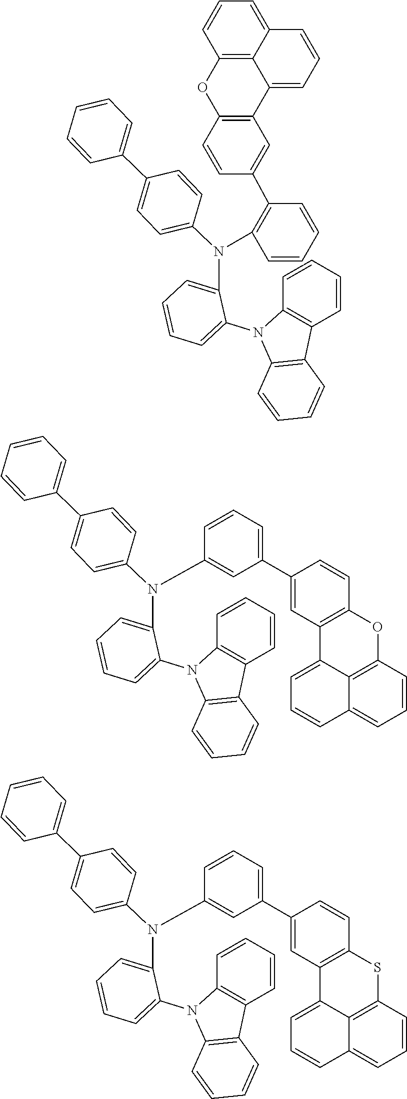 [SMILES: c1ccc(-c2ccc(N(c3ccccc3-c3ccc4c(c3)-c3cccc5cccc(c35)O4)c3ccccc3-n3c4ccccc4c4ccccc43)cc2)cc1 ; c1ccc(-c2ccc(N(c3cccc(-c4ccc5c(c4)-c4cccc6cccc(c46)O5)c3)c3ccccc3-n3c4ccccc4c4ccccc43)cc2)cc1 ; c1ccc(-c2ccc(N(c3cccc(-c4ccc5c(c4)-c4cccc6cccc(c46)S5)c3)c3ccccc3-n3c4ccccc4c4ccccc43)cc2)cc1]

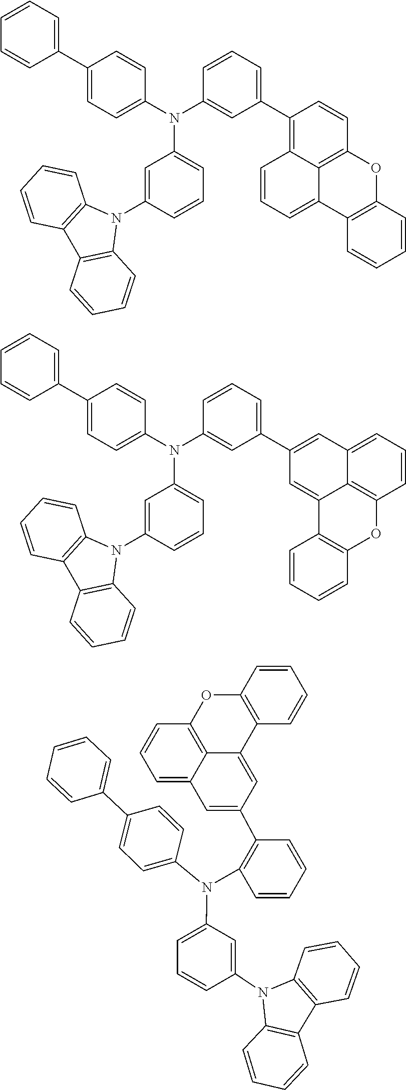 [SMILES: c1ccc(-c2ccc(N(c3cccc(-c4ccc5c6c(cccc46)-c4ccccc4O5)c3)c3cccc(-n4c5ccccc5c5ccccc54)c3)cc2)cc1 ; c1ccc(-c2ccc(N(c3cccc(-c4cc5c6c(cccc6c4)Oc4ccccc4-5)c3)c3cccc(-n4c5ccccc5c5ccccc54)c3)cc2)cc1 ; c1ccc(-c2ccc(N(c3cccc(-n4c5ccccc5c5ccccc54)c3)c3ccccc3-c3cc4c5c(cccc5c3)Oc3ccccc3-4)cc2)cc1]

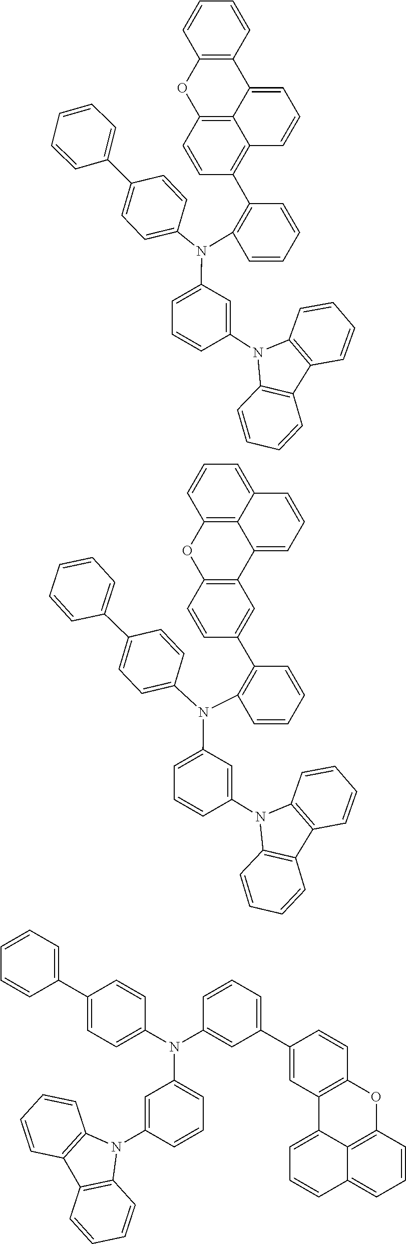

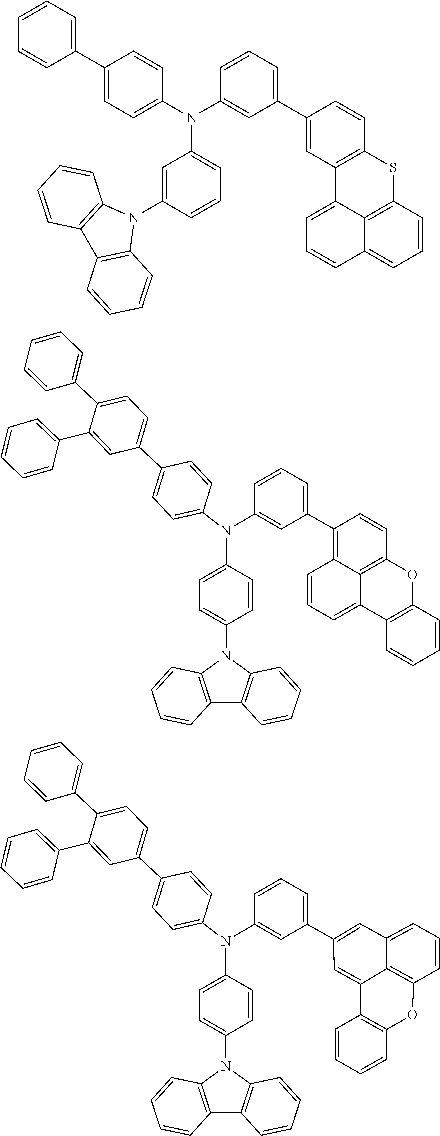 [SMILES: c1ccc(-c2ccc(N(c3cccc(-c4ccc5c(c4)-c4cccc6cccc(c46)S5)c3)c3cccc(-n4c5ccccc5c5ccccc54)c3)cc2)cc1 ; c1ccc(-c2ccc(-c3ccc(N(c4ccc(-n5c6ccccc6c6ccccc65)cc4)c4cccc(-c5ccc6c7c(cccc57)-c5ccccc5O6)c4)cc3)cc2-c2ccccc2)cc1 ; c1ccc(-c2ccc(-c3ccc(N(c4ccc(-n5c6ccccc6c6ccccc65)cc4)c4cccc(-c5cc6c7c(cccc7c5)Oc5ccccc5-6)c4)cc3)cc2-c2ccccc2)cc1]

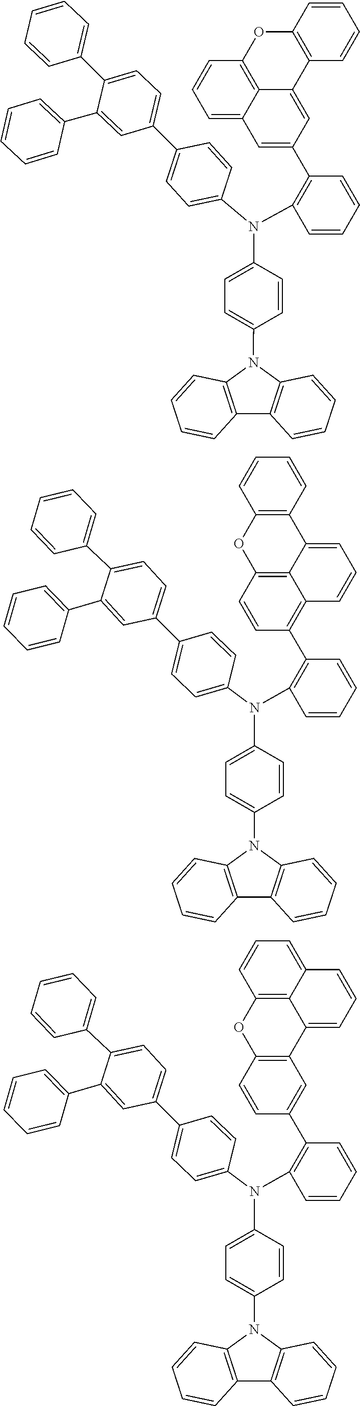

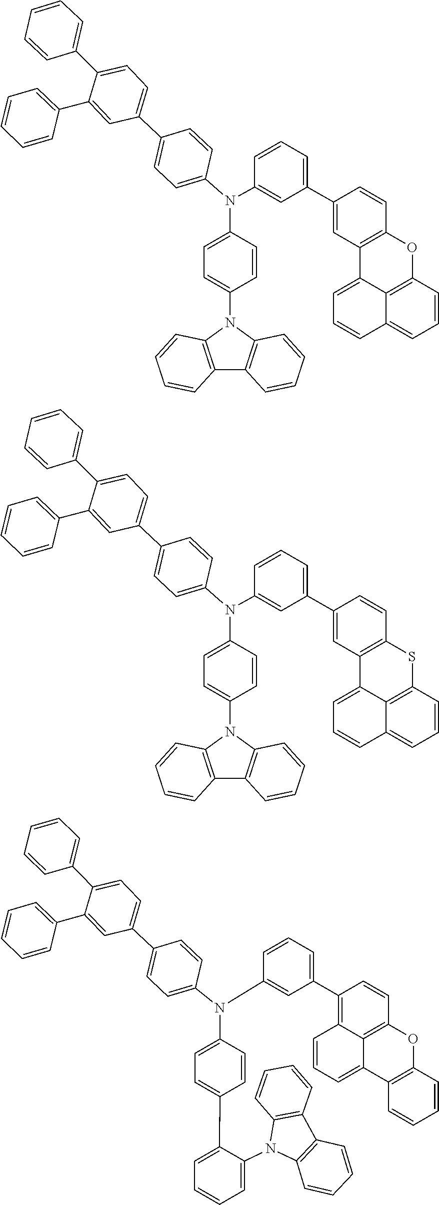 [SMILES: c1ccc(-c2ccc(-c3ccc(N(c4ccc(-n5c6ccccc6c6ccccc65)cc4)c4cccc(-c5ccc6c(c5)-c5cccc7cccc(c57)O6)c4)cc3)cc2-c2ccccc2)cc1 ; c1ccc(-c2ccc(-c3ccc(N(c4ccc(-n5c6ccccc6c6ccccc65)cc4)c4cccc(-c5ccc6c(c5)-c5cccc7cccc(c57)S6)c4)cc3)cc2-c2ccccc2)cc1 ; c1ccc(-c2ccc(-c3ccc(N(c4ccc(-c5ccccc5-n5c6ccccc6c6ccccc65)cc4)c4cccc(-c5ccc6c7c(cccc57)-c5ccccc5O6)c4)cc3)cc2-c2ccccc2)cc1]

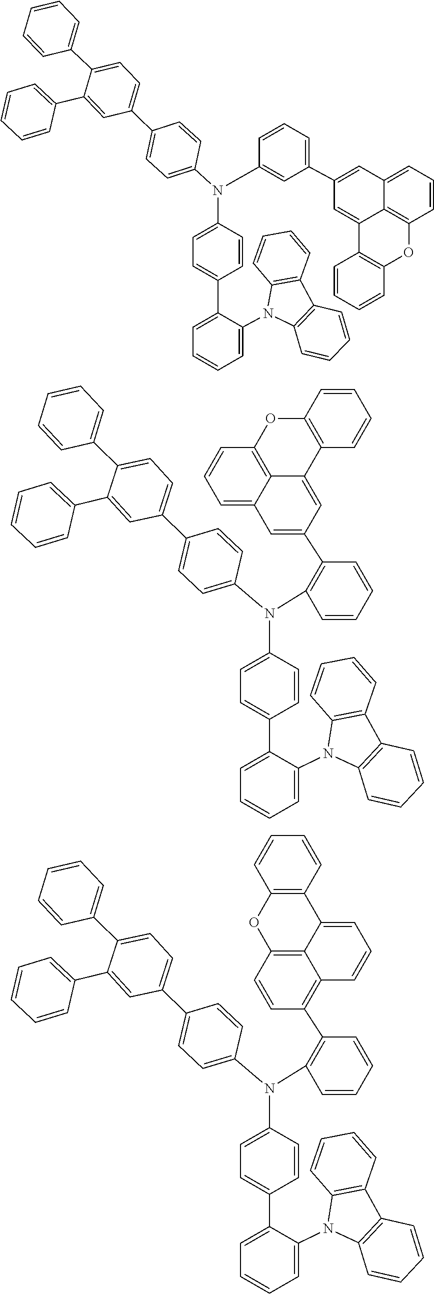

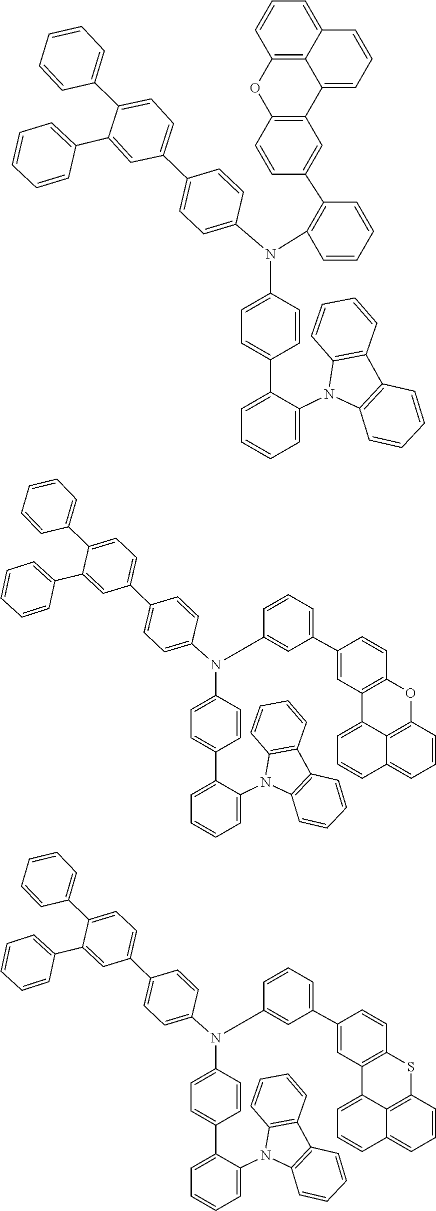 [SMILES: c1ccc(-c2ccc(-c3ccc(N(c4ccc(-c5ccccc5-n5c6ccccc6c6ccccc65)cc4)c4ccccc4-c4ccc5c(c4)-c4cccc6cccc(c46)O5)cc3)cc2-c2ccccc2)cc1 ; c1ccc(-c2ccc(-c3ccc(N(c4ccc(-c5ccccc5-n5c6ccccc6c6ccccc65)cc4)c4cccc(-c5ccc6c(c5)-c5cccc7cccc(c57)O6)c4)cc3)cc2-c2ccccc2)cc1 ; c1ccc(-c2ccc(-c3ccc(N(c4ccc(-c5ccccc5-n5c6ccccc6c6ccccc65)cc4)c4cccc(-c5ccc6c(c5)-c5cccc7cccc(c57)S6)c4)cc3)cc2-c2ccccc2)cc1]

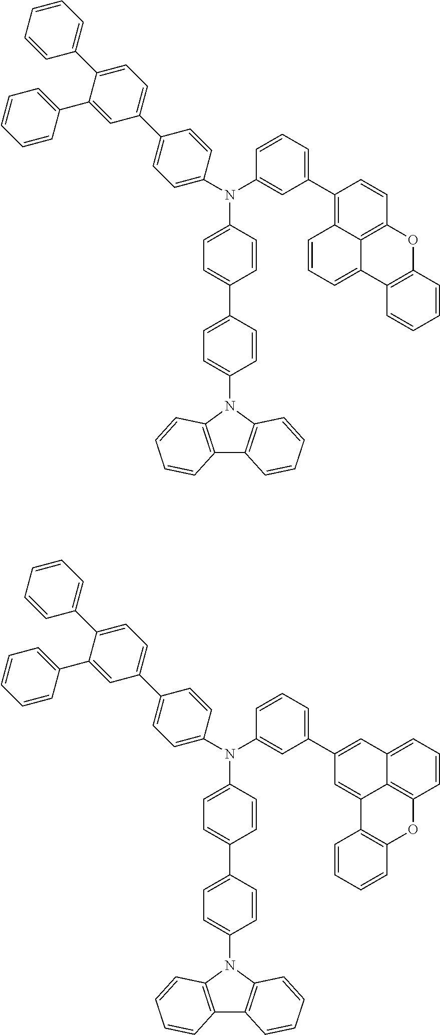 [SMILES: c1ccc(-c2ccc(-c3ccc(N(c4ccc(-c5ccc(-n6c7ccccc7c7ccccc76)cc5)cc4)c4cccc(-c5ccc6c7c(cccc57)-c5ccccc5O6)c4)cc3)cc2-c2ccccc2)cc1 ; c1ccc(-c2ccc(-c3ccc(N(c4ccc(-c5ccc(-n6c7ccccc7c7ccccc76)cc5)cc4)c4cccc(-c5cc6c7c(cccc7c5)Oc5ccccc5-6)c4)cc3)cc2-c2ccccc2)cc1]

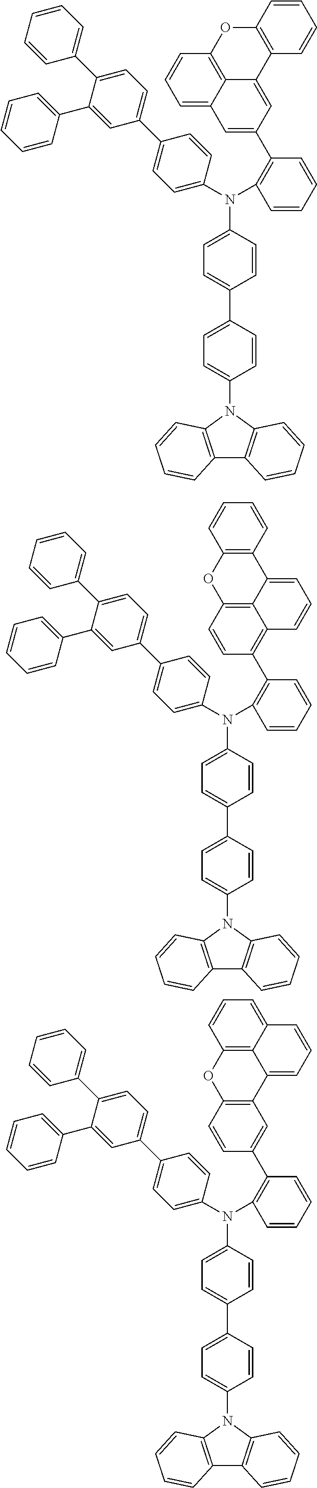

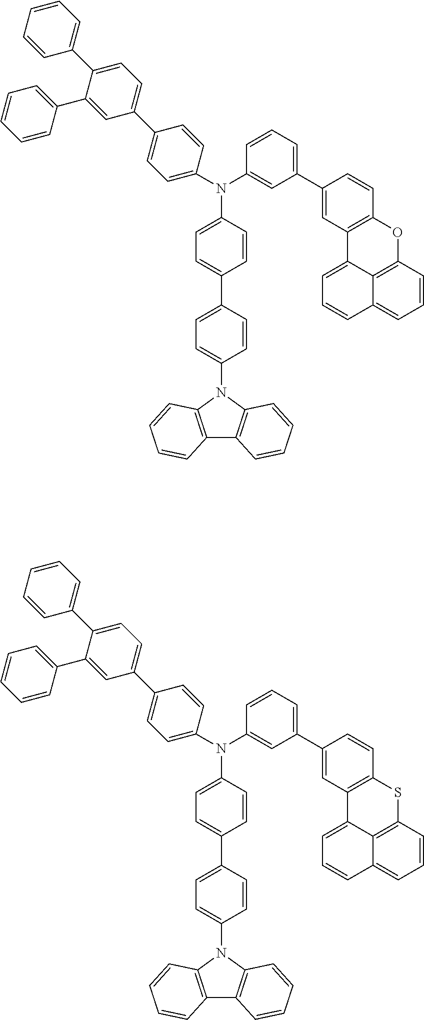 [SMILES: c1ccc(-c2ccc(-c3ccc(N(c4ccc(-c5ccc(-n6c7ccccc7c7ccccc76)cc5)cc4)c4cccc(-c5ccc6c(c5)-c5cccc7cccc(c57)O6)c4)cc3)cc2-c2ccccc2)cc1 ; c1ccc(-c2ccc(-c3ccc(N(c4ccc(-c5ccc(-n6c7ccccc7c7ccccc76)cc5)cc4)c4cccc(-c5ccc6c(c5)-c5cccc7cccc(c57)S6)c4)cc3)cc2-c2ccccc2)cc1]

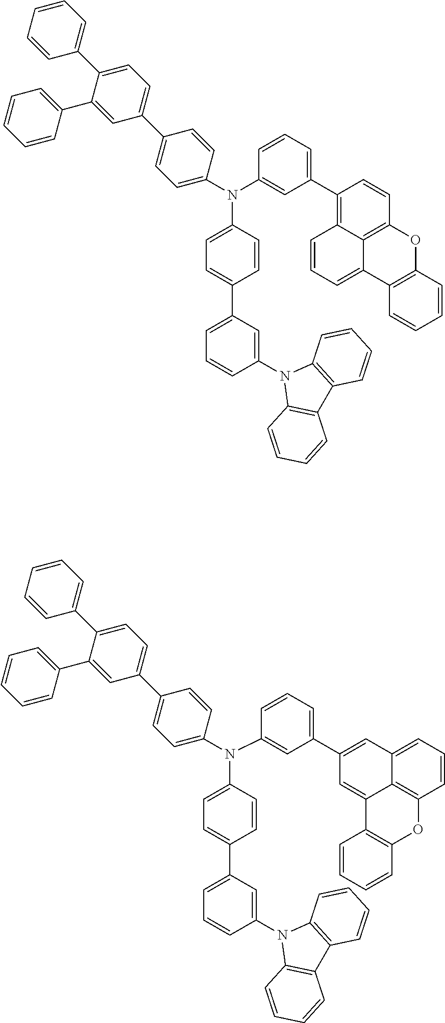 [SMILES: c1ccc(-c2ccc(-c3ccc(N(c4ccc(-c5cccc(-n6c7ccccc7c7ccccc76)c5)cc4)c4cccc(-c5ccc6c7c(cccc57)-c5ccccc5O6)c4)cc3)cc2-c2ccccc2)cc1 ; c1ccc(-c2ccc(-c3ccc(N(c4ccc(-c5cccc(-n6c7ccccc7c7ccccc76)c5)cc4)c4cccc(-c5cc6c7c(cccc7c5)Oc5ccccc5-6)c4)cc3)cc2-c2ccccc2)cc1]

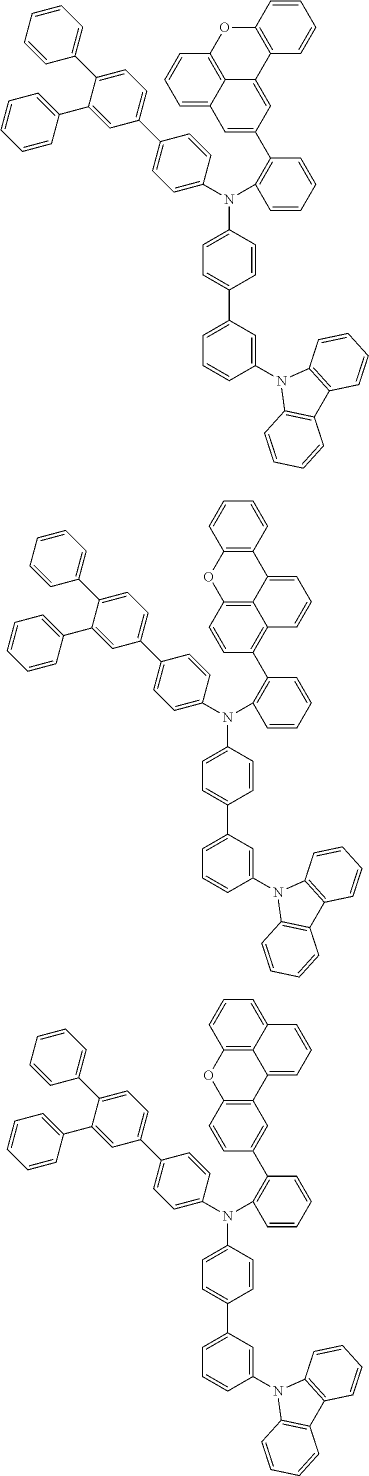

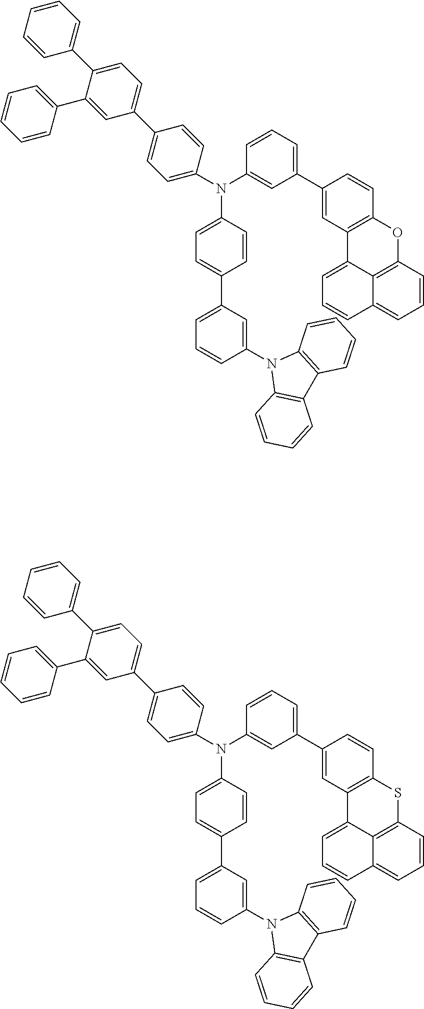 [SMILES: c1ccc(-c2ccc(-c3ccc(N(c4ccc(-c5cccc(-n6c7ccccc7c7ccccc76)c5)cc4)c4cccc(-c5ccc6c(c5)-c5cccc7cccc(c57)O6)c4)cc3)cc2-c2ccccc2)cc1 ; c1ccc(-c2ccc(-c3ccc(N(c4ccc(-c5cccc(-n6c7ccccc7c7ccccc76)c5)cc4)c4cccc(-c5ccc6c(c5)-c5cccc7cccc(c57)S6)c4)cc3)cc2-c2ccccc2)cc1]

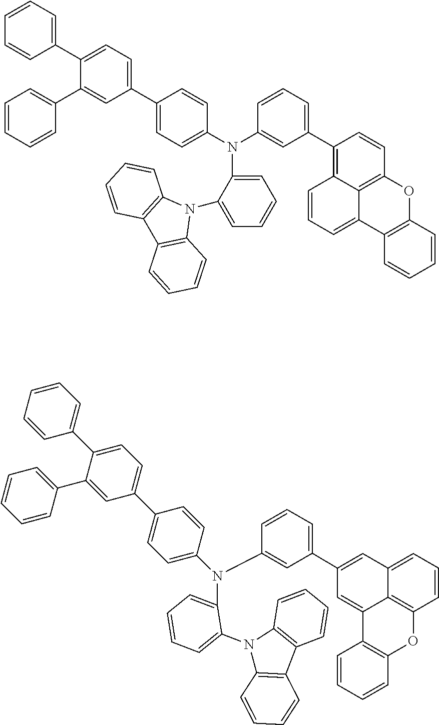 [SMILES: c1ccc(-c2ccc(-c3ccc(N(c4cccc(-c5ccc6c7c(cccc57)-c5ccccc5O6)c4)c4ccccc4-n4c5ccccc5c5ccccc54)cc3)cc2-c2ccccc2)cc1 ; c1ccc(-c2ccc(-c3ccc(N(c4cccc(-c5cc6c7c(cccc7c5)Oc5ccccc5-6)c4)c4ccccc4-n4c5ccccc5c5ccccc54)cc3)cc2-c2ccccc2)cc1]

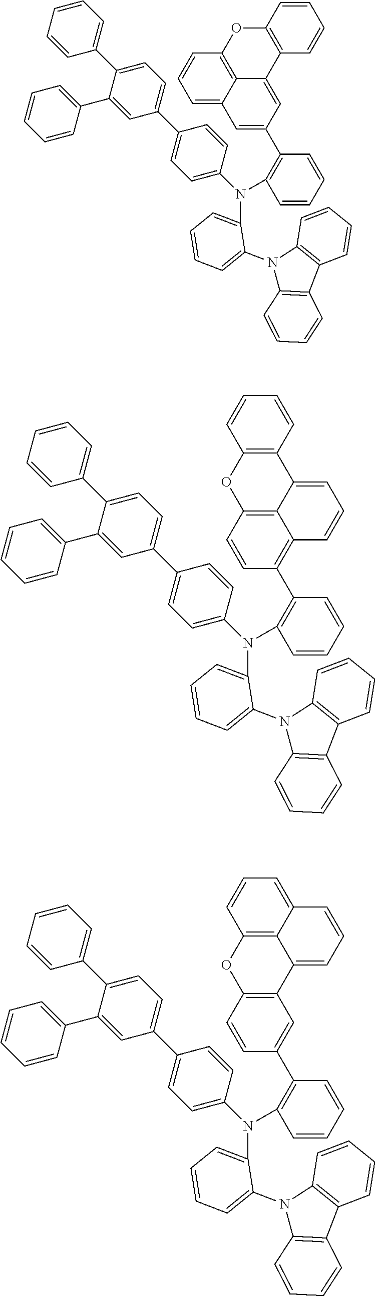 [SMILES: c1ccc(-c2ccc(-c3ccc(N(c4ccccc4-c4cc5c6c(cccc6c4)Oc4ccccc4-5)c4ccccc4-n4c5ccccc5c5ccccc54)cc3)cc2-c2ccccc2)cc1 ; c1ccc(-c2ccc(-c3ccc(N(c4ccccc4-c4ccc5c6c(cccc46)-c4ccccc4O5)c4ccccc4-n4c5ccccc5c5ccccc54)cc3)cc2-c2ccccc2)cc1 ; c1ccc(-c2ccc(-c3ccc(N(c4ccccc4-c4ccc5c(c4)-c4cccc6cccc(c46)O5)c4ccccc4-n4c5ccccc5c5ccccc54)cc3)cc2-c2ccccc2)cc1]

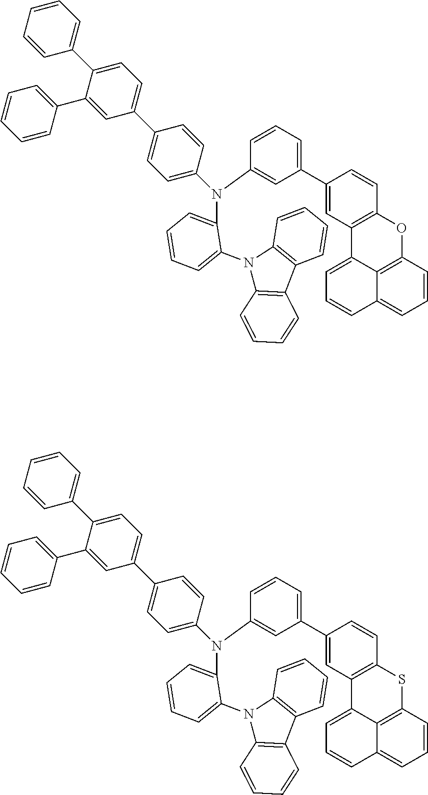 [SMILES: c1ccc(-c2ccc(-c3ccc(N(c4cccc(-c5ccc6c(c5)-c5cccc7cccc(c57)O6)c4)c4ccccc4-n4c5ccccc5c5ccccc54)cc3)cc2-c2ccccc2)cc1 ; c1ccc(-c2ccc(-c3ccc(N(c4cccc(-c5ccc6c(c5)-c5cccc7cccc(c57)S6)c4)c4ccccc4-n4c5ccccc5c5ccccc54)cc3)cc2-c2ccccc2)cc1]

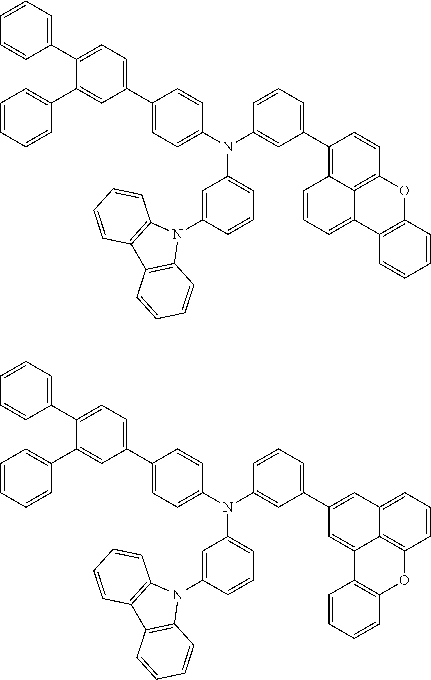 [SMILES: c1ccc(-c2ccc(-c3ccc(N(c4cccc(-c5ccc6c7c(cccc57)-c5ccccc5O6)c4)c4cccc(-n5c6ccccc6c6ccccc65)c4)cc3)cc2-c2ccccc2)cc1 ; c1ccc(-c2ccc(-c3ccc(N(c4cccc(-c5cc6c7c(cccc7c5)Oc5ccccc5-6)c4)c4cccc(-n5c6ccccc6c6ccccc65)c4)cc3)cc2-c2ccccc2)cc1]

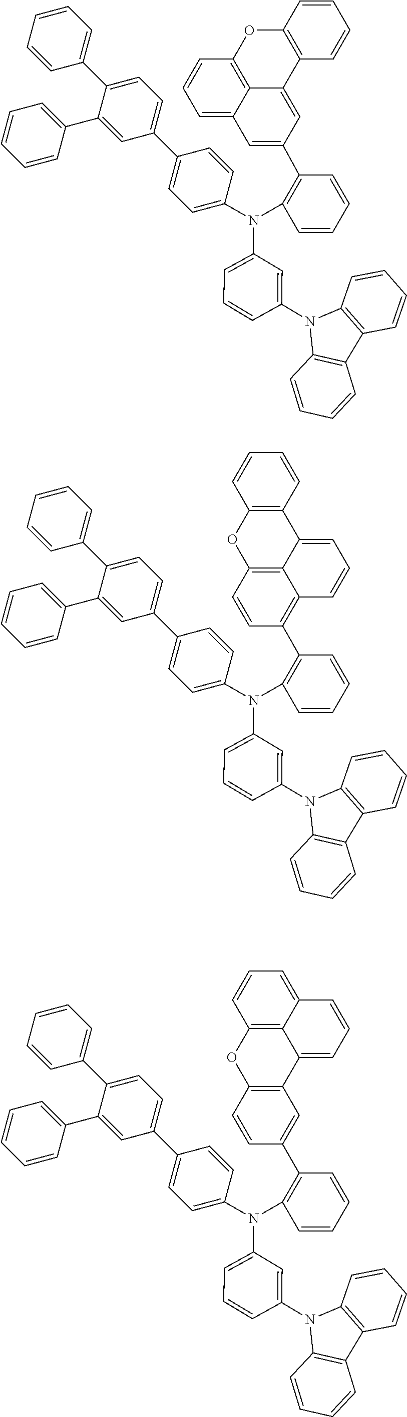

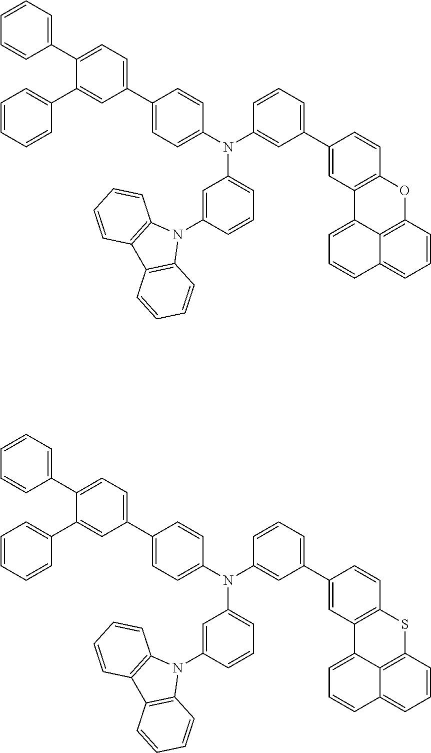 [SMILES: c1ccc(-c2ccc(-c3ccc(N(c4cccc(-c5ccc6c(c5)-c5cccc7cccc(c57)O6)c4)c4cccc(-n5c6ccccc6c6ccccc65)c4)cc3)cc2-c2ccccc2)cc1 ; c1ccc(-c2ccc(-c3ccc(N(c4cccc(-c5ccc6c(c5)-c5cccc7cccc(c57)S6)c4)c4cccc(-n5c6ccccc6c6ccccc65)c4)cc3)cc2-c2ccccc2)cc1]

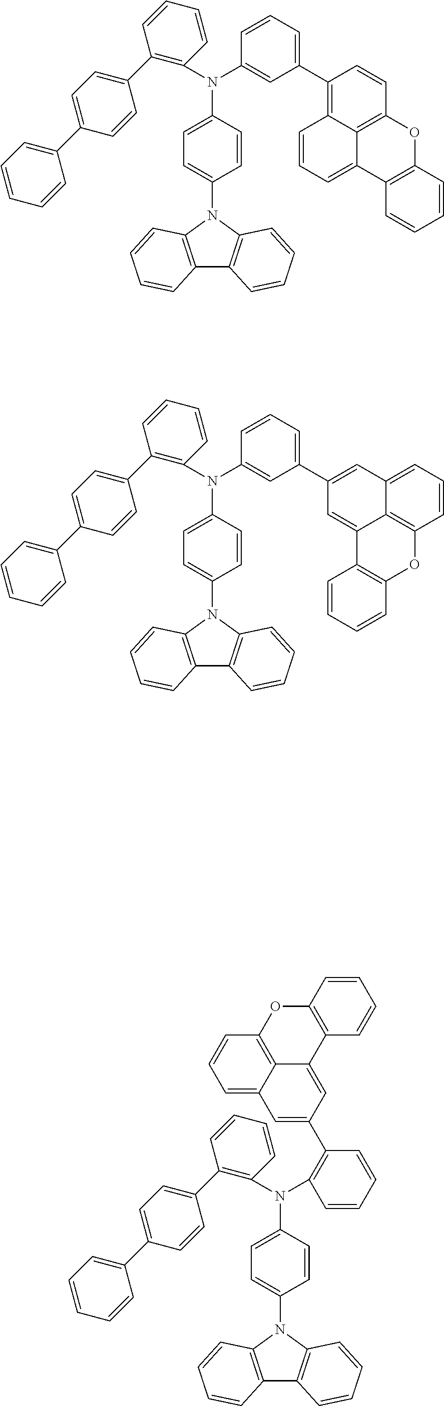 [SMILES: c1ccc(-c2ccc(-c3ccccc3N(c3ccc(-n4c5ccccc5c5ccccc54)cc3)c3cccc(-c4ccc5c6c(cccc46)-c4ccccc4O5)c3)cc2)cc1 ; c1ccc(-c2ccc(-c3ccccc3N(c3ccc(-n4c5ccccc5c5ccccc54)cc3)c3cccc(-c4cc5c6c(cccc6c4)Oc4ccccc4-5)c3)cc2)cc1 ; c1ccc(-c2ccc(-c3ccccc3N(c3ccc(-n4c5ccccc5c5ccccc54)cc3)c3ccccc3-c3cc4c5c(cccc5c3)Oc3ccccc3-4)cc2)cc1]

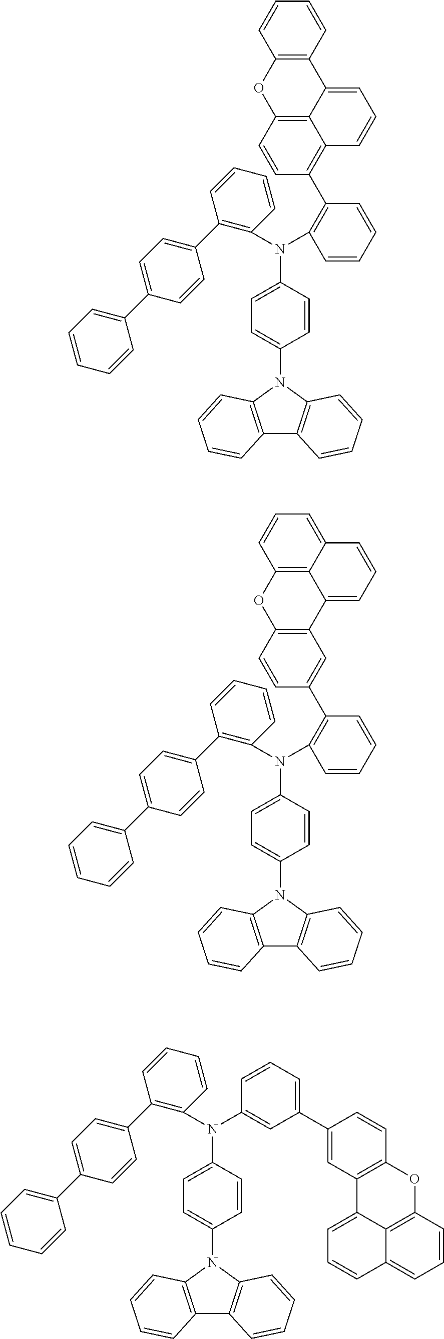 [SMILES: c1ccc(-c2ccc(-c3ccccc3N(c3ccc(-n4c5ccccc5c5ccccc54)cc3)c3ccccc3-c3ccc4c5c(cccc35)-c3ccccc3O4)cc2)cc1 ; c1ccc(-c2ccc(-c3ccccc3N(c3ccc(-n4c5ccccc5c5ccccc54)cc3)c3ccccc3-c3ccc4c(c3)-c3cccc5cccc(c35)O4)cc2)cc1 ; c1ccc(-c2ccc(-c3ccccc3N(c3ccc(-n4c5ccccc5c5ccccc54)cc3)c3cccc(-c4ccc5c(c4)-c4cccc6cccc(c46)O5)c3)cc2)cc1]

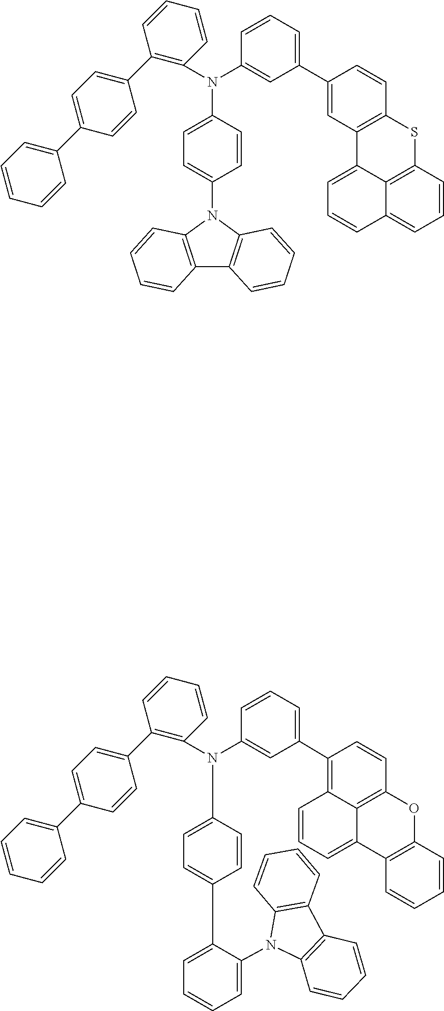 [SMILES: c1ccc(-c2ccc(-c3ccccc3N(c3ccc(-n4c5ccccc5c5ccccc54)cc3)c3cccc(-c4ccc5c(c4)-c4cccc6cccc(c46)S5)c3)cc2)cc1 ; c1ccc(-c2ccc(-c3ccccc3N(c3ccc(-c4ccccc4-n4c5ccccc5c5ccccc54)cc3)c3cccc(-c4ccc5c6c(cccc46)-c4ccccc4O5)c3)cc2)cc1]

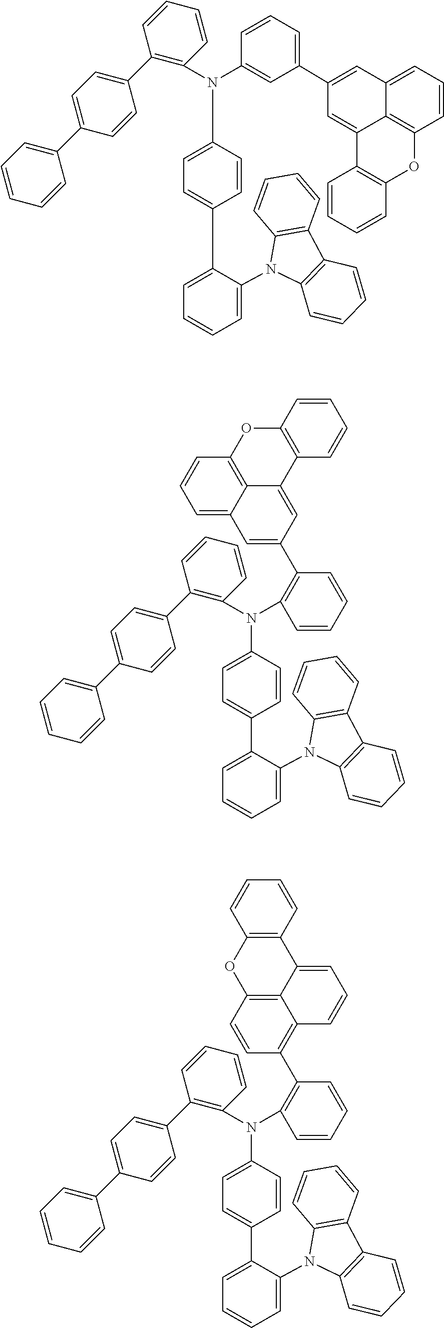 [SMILES: c1ccc(-c2ccc(-c3ccccc3N(c3ccc(-c4ccccc4-n4c5ccccc5c5ccccc54)cc3)c3cccc(-c4cc5c6c(cccc6c4)Oc4ccccc4-5)c3)cc2)cc1 ; c1ccc(-c2ccc(-c3ccccc3N(c3ccc(-c4ccccc4-n4c5ccccc5c5ccccc54)cc3)c3ccccc3-c3cc4c5c(cccc5c3)Oc3ccccc3-4)cc2)cc1 ; c1ccc(-c2ccc(-c3ccccc3N(c3ccc(-c4ccccc4-n4c5ccccc5c5ccccc54)cc3)c3ccccc3-c3ccc4c5c(cccc35)-c3ccccc3O4)cc2)cc1]

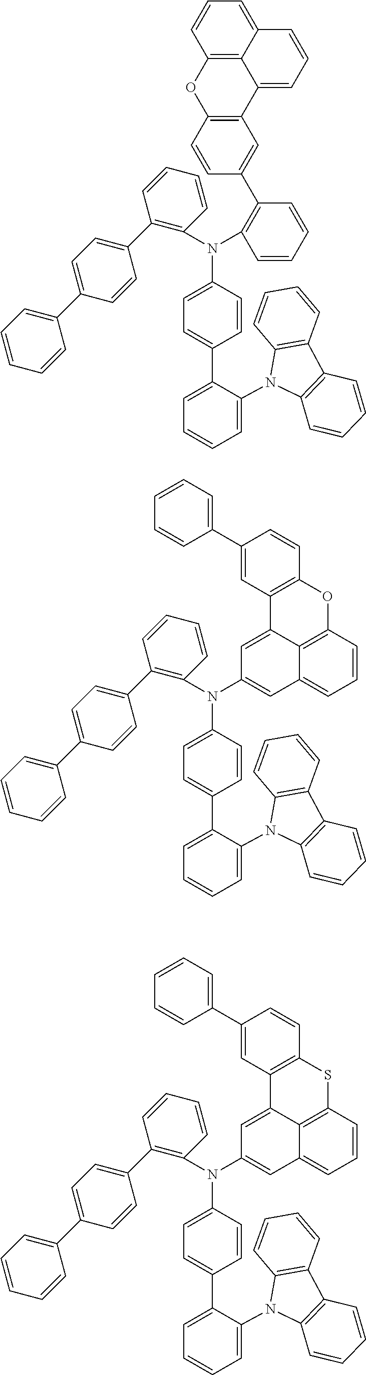

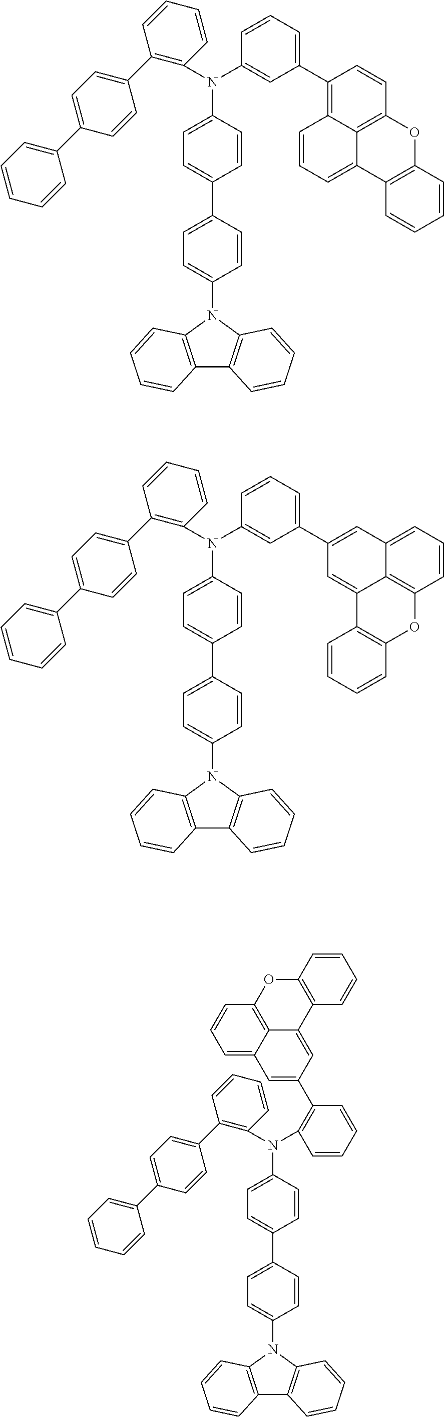 [SMILES: c1ccc(-c2ccc(-c3ccccc3N(c3ccc(-c4ccc(-n5c6ccccc6c6ccccc65)cc4)cc3)c3cccc(-c4ccc5c6c(cccc46)-c4ccccc4O5)c3)cc2)cc1 ; c1ccc(-c2ccc(-c3ccccc3N(c3ccc(-c4ccc(-n5c6ccccc6c6ccccc65)cc4)cc3)c3cccc(-c4cc5c6c(cccc6c4)Oc4ccccc4-5)c3)cc2)cc1 ; c1ccc(-c2ccc(-c3ccccc3N(c3ccc(-c4ccc(-n5c6ccccc6c6ccccc65)cc4)cc3)c3ccccc3-c3cc4c5c(cccc5c3)Oc3ccccc3-4)cc2)cc1]

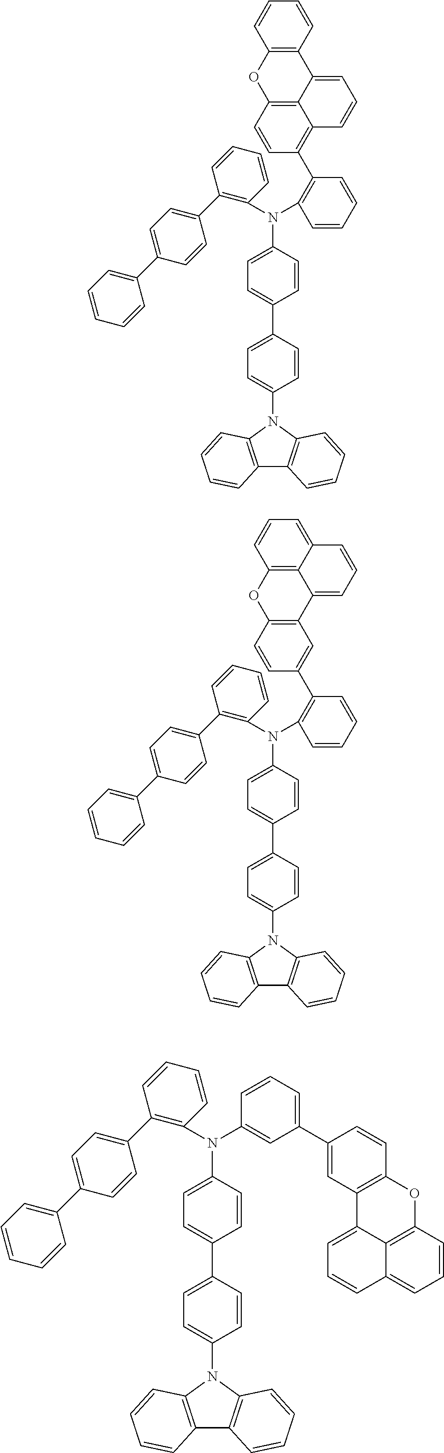

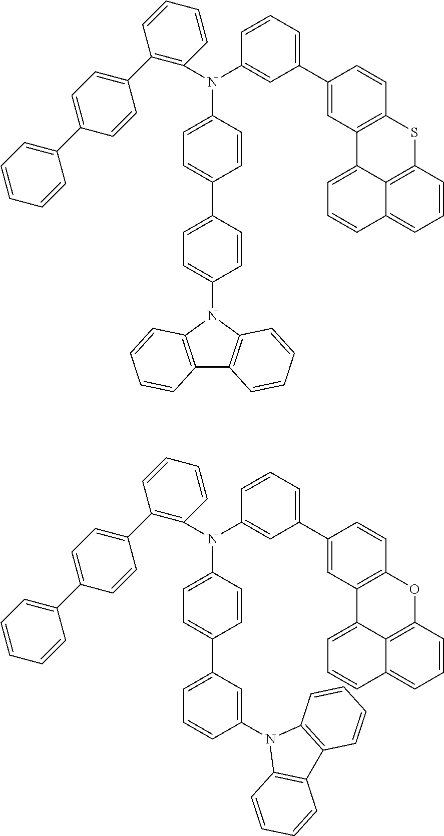 [SMILES: c1ccc(-c2ccc(-c3ccccc3N(c3ccc(-c4ccc(-n5c6ccccc6c6ccccc65)cc4)cc3)c3cccc(-c4ccc5c(c4)-c4cccc6cccc(c46)S5)c3)cc2)cc1 ; c1ccc(-c2ccc(-c3ccccc3N(c3ccc(-c4cccc(-n5c6ccccc6c6ccccc65)c4)cc3)c3cccc(-c4ccc5c(c4)-c4cccc6cccc(c46)O5)c3)cc2)cc1]

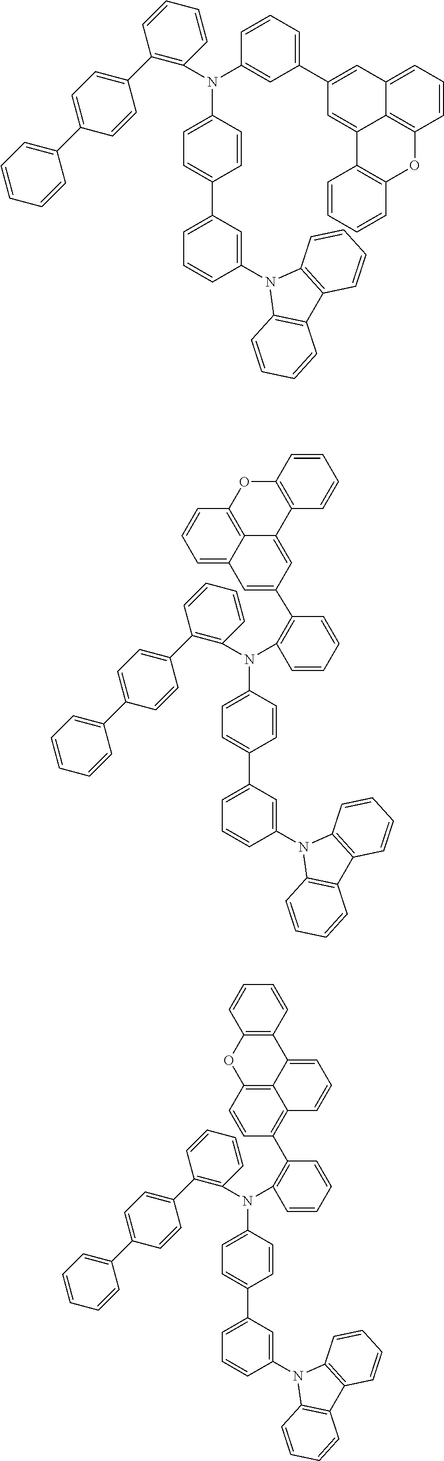 [SMILES: c1ccc(-c2ccc(-c3ccccc3N(c3ccc(-c4cccc(-n5c6ccccc6c6ccccc65)c4)cc3)c3cccc(-c4cc5c6c(cccc6c4)Oc4ccccc4-5)c3)cc2)cc1 ; c1ccc(-c2ccc(-c3ccccc3N(c3ccc(-c4cccc(-n5c6ccccc6c6ccccc65)c4)cc3)c3ccccc3-c3cc4c5c(cccc5c3)Oc3ccccc3-4)cc2)cc1 ; c1ccc(-c2ccc(-c3ccccc3N(c3ccc(-c4cccc(-n5c6ccccc6c6ccccc65)c4)cc3)c3ccccc3-c3ccc4c5c(cccc35)-c3ccccc3O4)cc2)cc1]

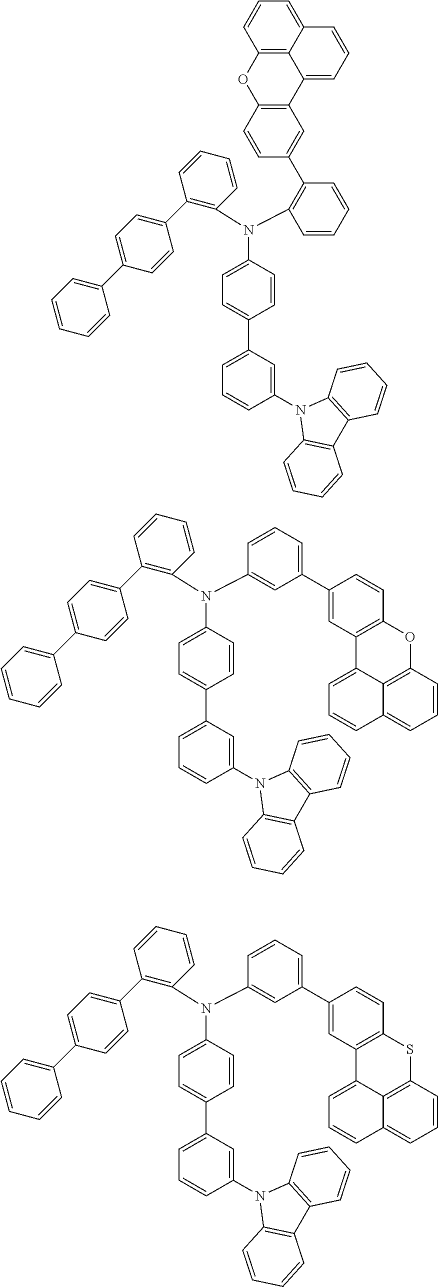

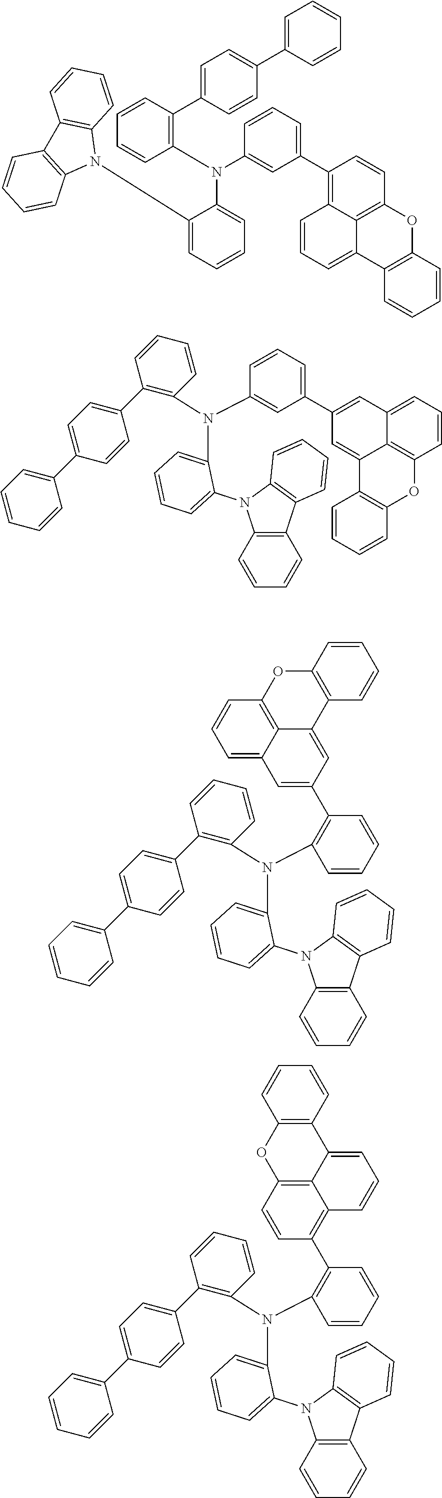

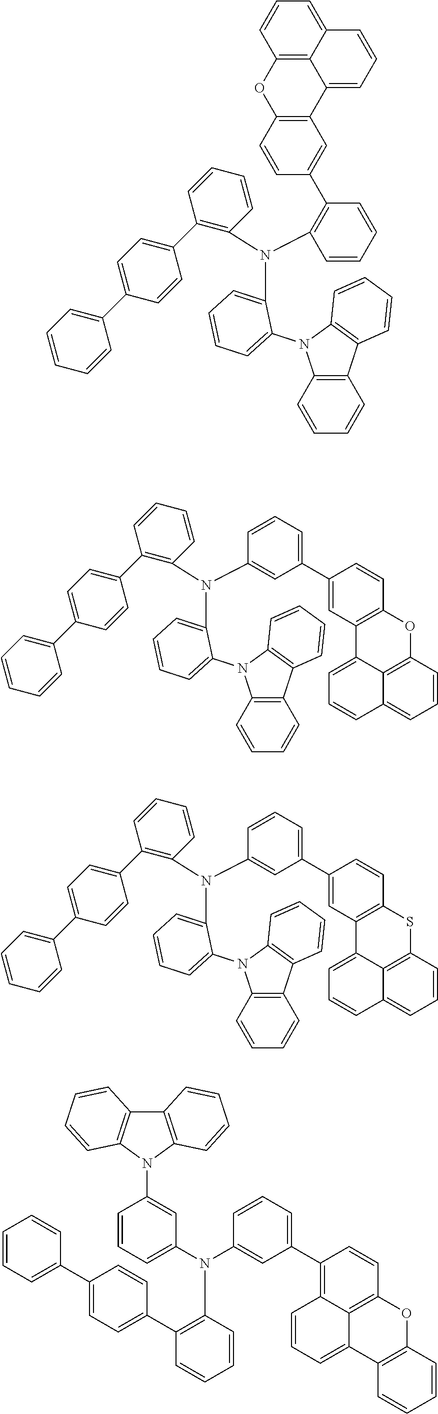 [SMILES: c1ccc(-c2ccc(-c3ccccc3N(c3ccccc3-c3ccc4c(c3)-c3cccc5cccc(c35)O4)c3ccccc3-n3c4ccccc4c4ccccc43)cc2)cc1 ; c1ccc(-c2ccc(-c3ccccc3N(c3cccc(-c4ccc5c(c4)-c4cccc6cccc(c46)O5)c3)c3ccccc3-n3c4ccccc4c4ccccc43)cc2)cc1 ; c1ccc(-c2ccc(-c3ccccc3N(c3cccc(-c4ccc5c(c4)-c4cccc6cccc(c46)S5)c3)c3ccccc3-n3c4ccccc4c4ccccc43)cc2)cc1 ; c1ccc(-c2ccc(-c3ccccc3N(c3cccc(-c4ccc5c6c(cccc46)-c4ccccc4O5)c3)c3cccc(-n4c5ccccc5c5ccccc54)c3)cc2)cc1]

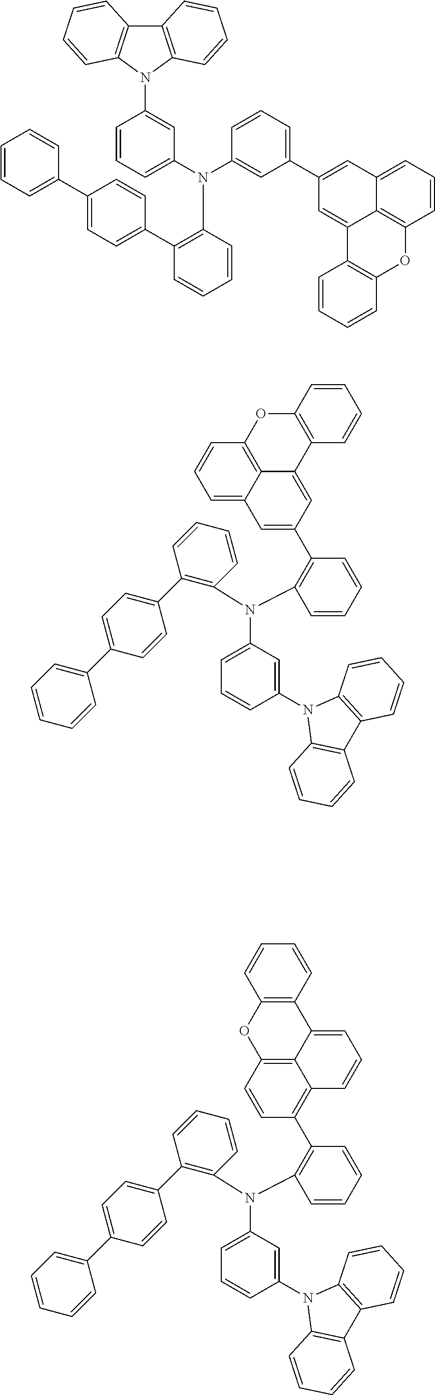 [SMILES: c1ccc(-c2ccc(-c3ccccc3N(c3cccc(-c4cc5c6c(cccc6c4)Oc4ccccc4-5)c3)c3cccc(-n4c5ccccc5c5ccccc54)c3)cc2)cc1 ; c1ccc(-c2ccc(-c3ccccc3N(c3cccc(-n4c5ccccc5c5ccccc54)c3)c3ccccc3-c3cc4c5c(cccc5c3)Oc3ccccc3-4)cc2)cc1 ; c1ccc(-c2ccc(-c3ccccc3N(c3cccc(-n4c5ccccc5c5ccccc54)c3)c3ccccc3-c3ccc4c5c(cccc35)-c3ccccc3O4)cc2)cc1]

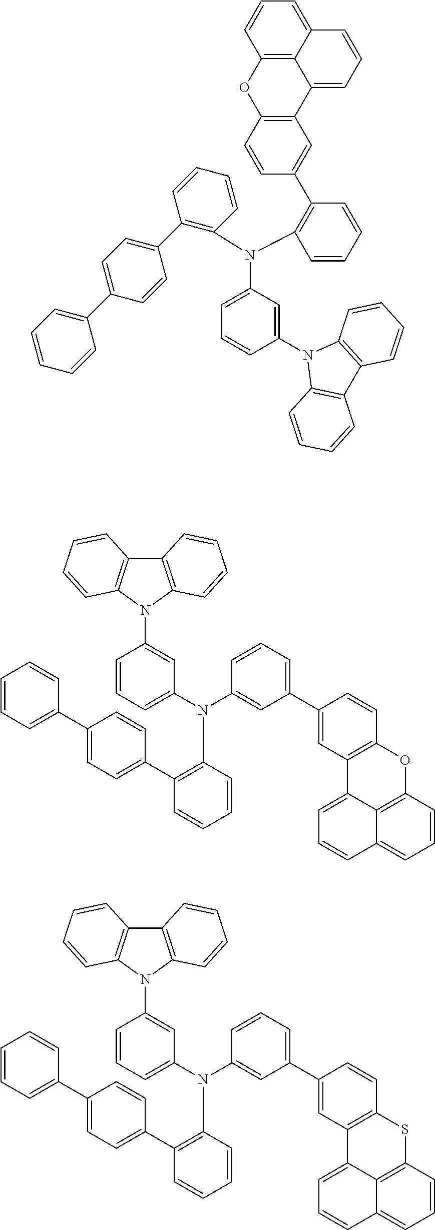 [SMILES: c1ccc(-c2ccc(-c3ccccc3N(c3cccc(-n4c5ccccc5c5ccccc54)c3)c3ccccc3-c3ccc4c(c3)-c3cccc5cccc(c35)O4)cc2)cc1 ; c1ccc(-c2ccc(-c3ccccc3N(c3cccc(-c4ccc5c(c4)-c4cccc6cccc(c46)O5)c3)c3cccc(-n4c5ccccc5c5ccccc54)c3)cc2)cc1 ; c1ccc(-c2ccc(-c3ccccc3N(c3cccc(-c4ccc5c(c4)-c4cccc6cccc(c46)S5)c3)c3cccc(-n4c5ccccc5c5ccccc54)c3)cc2)cc1]

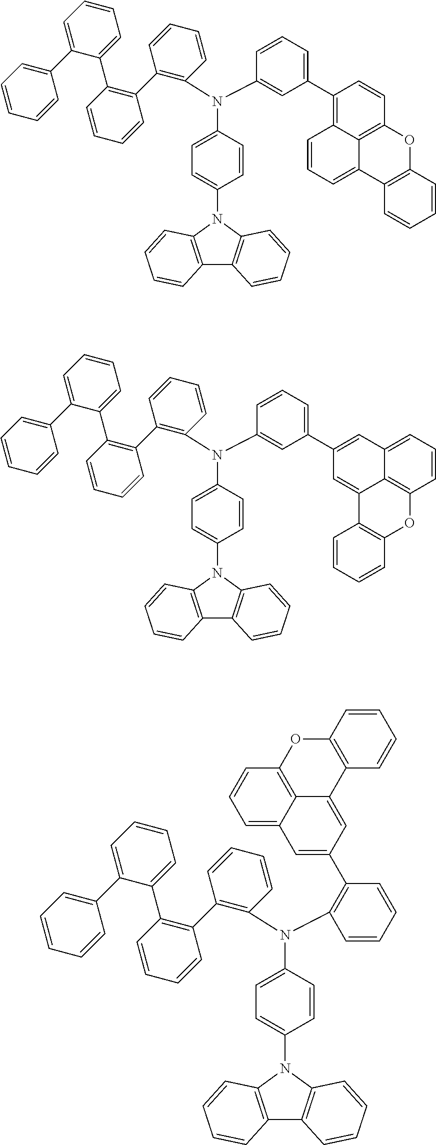 [SMILES: c1ccc(-c2ccccc2-c2ccccc2-c2ccccc2N(c2ccc(-n3c4ccccc4c4ccccc43)cc2)c2cccc(-c3ccc4c5c(cccc35)-c3ccccc3O4)c2)cc1 ; c1ccc(-c2ccccc2-c2ccccc2-c2ccccc2N(c2ccc(-n3c4ccccc4c4ccccc43)cc2)c2cccc(-c3cc4c5c(cccc5c3)Oc3ccccc3-4)c2)cc1 ; c1ccc(-c2ccccc2-c2ccccc2-c2ccccc2N(c2ccc(-n3c4ccccc4c4ccccc43)cc2)c2ccccc2-c2cc3c4c(cccc4c2)Oc2ccccc2-3)cc1]

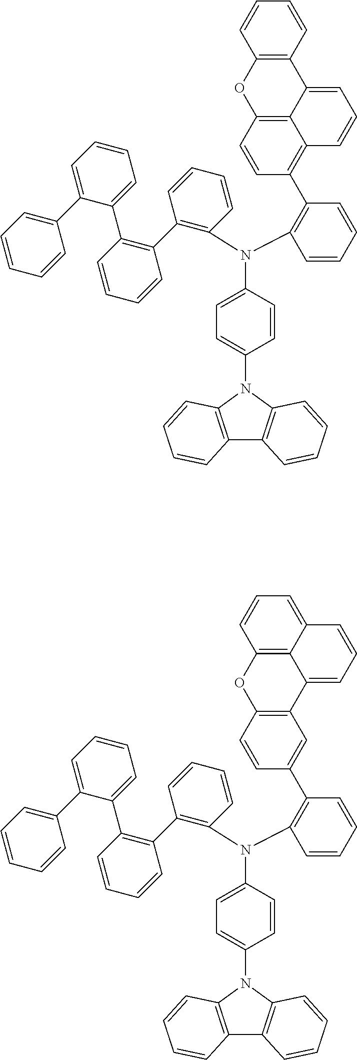 [SMILES: c1ccc(-c2ccccc2-c2ccccc2-c2ccccc2N(c2ccc(-n3c4ccccc4c4ccccc43)cc2)c2ccccc2-c2ccc3c4c(cccc24)-c2ccccc2O3)cc1 ; c1ccc(-c2ccccc2-c2ccccc2-c2ccccc2N(c2ccc(-n3c4ccccc4c4ccccc43)cc2)c2ccccc2-c2ccc3c(c2)-c2cccc4cccc(c24)O3)cc1]

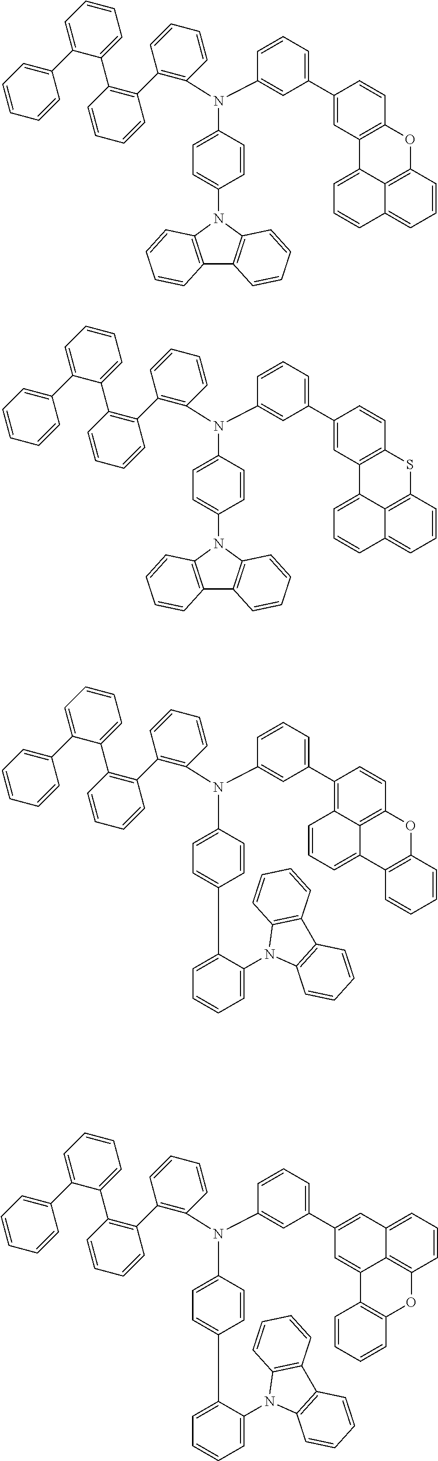 [SMILES: c1ccc(-c2ccccc2-c2ccccc2-c2ccccc2N(c2ccc(-n3c4ccccc4c4ccccc43)cc2)c2cccc(-c3ccc4c(c3)-c3cccc5cccc(c35)O4)c2)cc1 ; c1ccc(-c2ccccc2-c2ccccc2-c2ccccc2N(c2ccc(-n3c4ccccc4c4ccccc43)cc2)c2cccc(-c3ccc4c(c3)-c3cccc5cccc(c35)S4)c2)cc1 ; c1ccc(-c2ccccc2-c2ccccc2-c2ccccc2N(c2ccc(-c3ccccc3-n3c4ccccc4c4ccccc43)cc2)c2cccc(-c3ccc4c5c(cccc35)-c3ccccc3O4)c2)cc1 ; c1ccc(-c2ccccc2-c2ccccc2-c2ccccc2N(c2ccc(-c3ccccc3-n3c4ccccc4c4ccccc43)cc2)c2cccc(-c3cc4c5c(cccc5c3)Oc3ccccc3-4)c2)cc1]

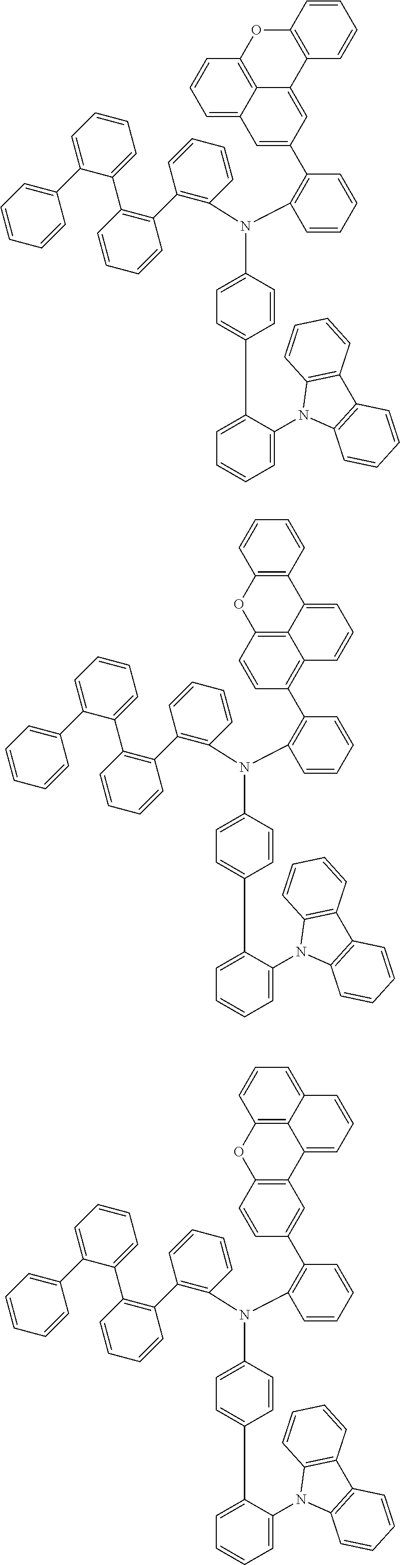

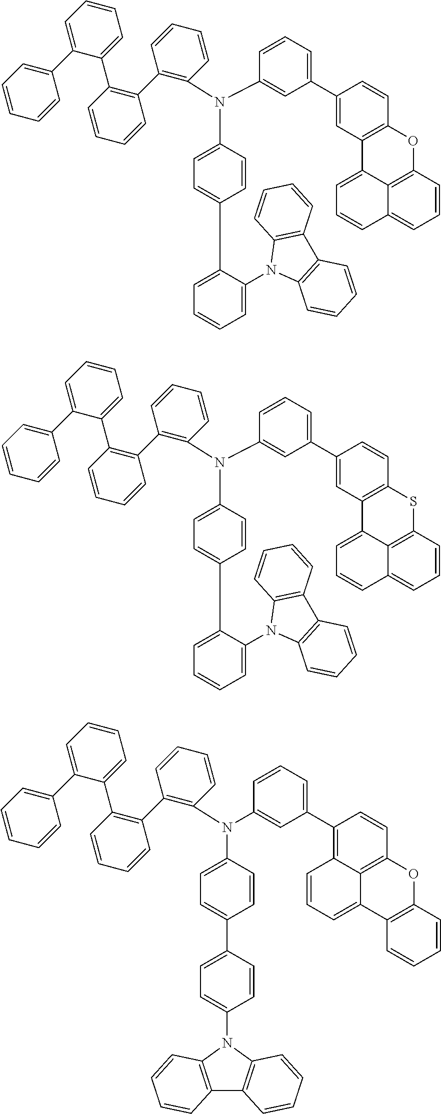 [SMILES: c1ccc(-c2ccccc2-c2ccccc2-c2ccccc2N(c2ccc(-c3ccccc3-n3c4ccccc4c4ccccc43)cc2)c2cccc(-c3ccc4c(c3)-c3cccc5cccc(c35)O4)c2)cc1 ; c1ccc(-c2ccccc2-c2ccccc2-c2ccccc2N(c2ccc(-c3ccccc3-n3c4ccccc4c4ccccc43)cc2)c2cccc(-c3ccc4c(c3)-c3cccc5cccc(c35)S4)c2)cc1 ; c1ccc(-c2ccccc2-c2ccccc2-c2ccccc2N(c2ccc(-c3ccc(-n4c5ccccc5c5ccccc54)cc3)cc2)c2cccc(-c3ccc4c5c(cccc35)-c3ccccc3O4)c2)cc1]

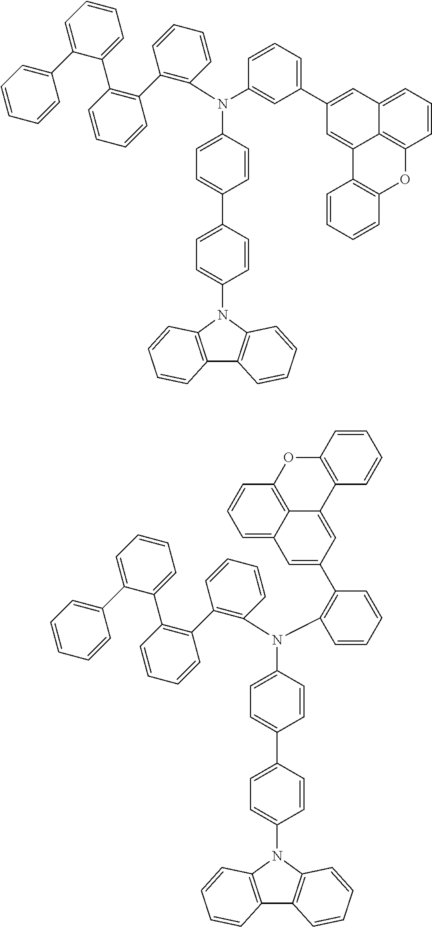 [SMILES: c1ccc(-c2ccccc2-c2ccccc2-c2ccccc2N(c2ccc(-c3ccc(-n4c5ccccc5c5ccccc54)cc3)cc2)c2cccc(-c3cc4c5c(cccc5c3)Oc3ccccc3-4)c2)cc1 ; c1ccc(-c2ccccc2-c2ccccc2-c2ccccc2N(c2ccc(-c3ccc(-n4c5ccccc5c5ccccc54)cc3)cc2)c2ccccc2-c2cc3c4c(cccc4c2)Oc2ccccc2-3)cc1]

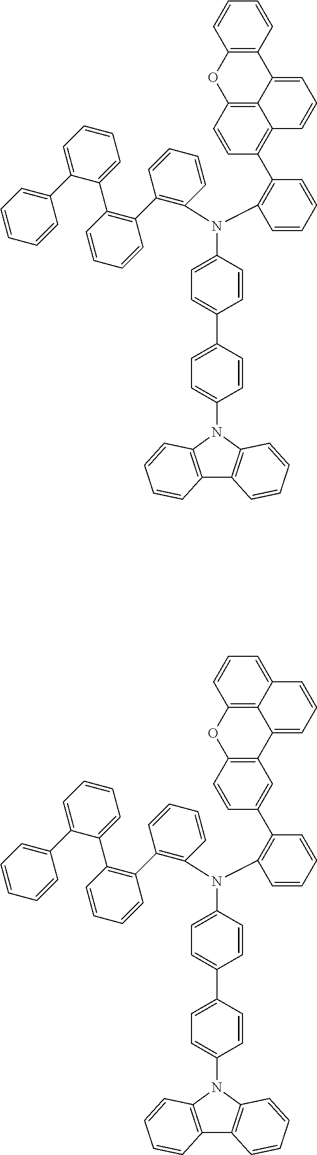 [SMILES: c1ccc(-c2ccccc2-c2ccccc2-c2ccccc2N(c2ccc(-c3ccc(-n4c5ccccc5c5ccccc54)cc3)cc2)c2ccccc2-c2ccc3c4c(cccc24)-c2ccccc2O3)cc1 ; c1ccc(-c2ccccc2-c2ccccc2-c2ccccc2N(c2ccc(-c3ccc(-n4c5ccccc5c5ccccc54)cc3)cc2)c2ccccc2-c2ccc3c(c2)-c2cccc4cccc(c24)O3)cc1]

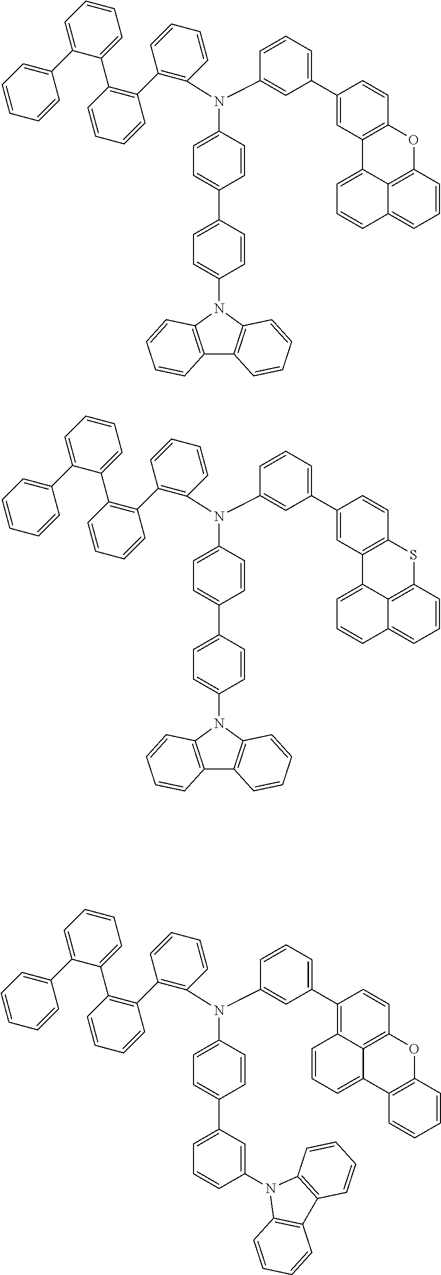 [SMILES: c1ccc(-c2ccccc2-c2ccccc2-c2ccccc2N(c2ccc(-c3ccc(-n4c5ccccc5c5ccccc54)cc3)cc2)c2cccc(-c3ccc4c(c3)-c3cccc5cccc(c35)O4)c2)cc1 ; c1ccc(-c2ccccc2-c2ccccc2-c2ccccc2N(c2ccc(-c3ccc(-n4c5ccccc5c5ccccc54)cc3)cc2)c2cccc(-c3ccc4c(c3)-c3cccc5cccc(c35)S4)c2)cc1 ; c1ccc(-c2ccccc2-c2ccccc2-c2ccccc2N(c2ccc(-c3cccc(-n4c5ccccc5c5ccccc54)c3)cc2)c2cccc(-c3ccc4c5c(cccc35)-c3ccccc3O4)c2)cc1]

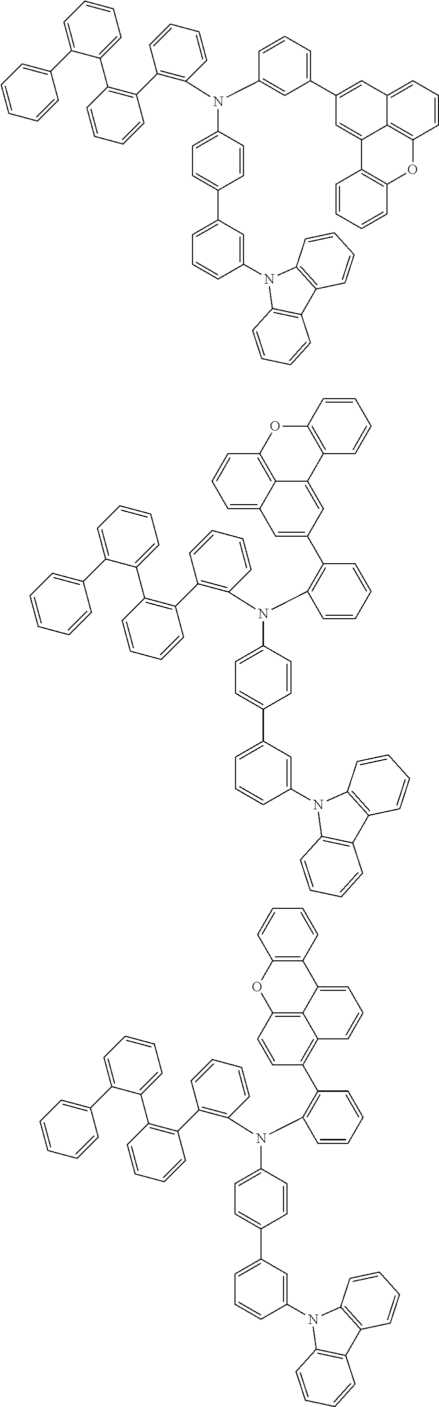

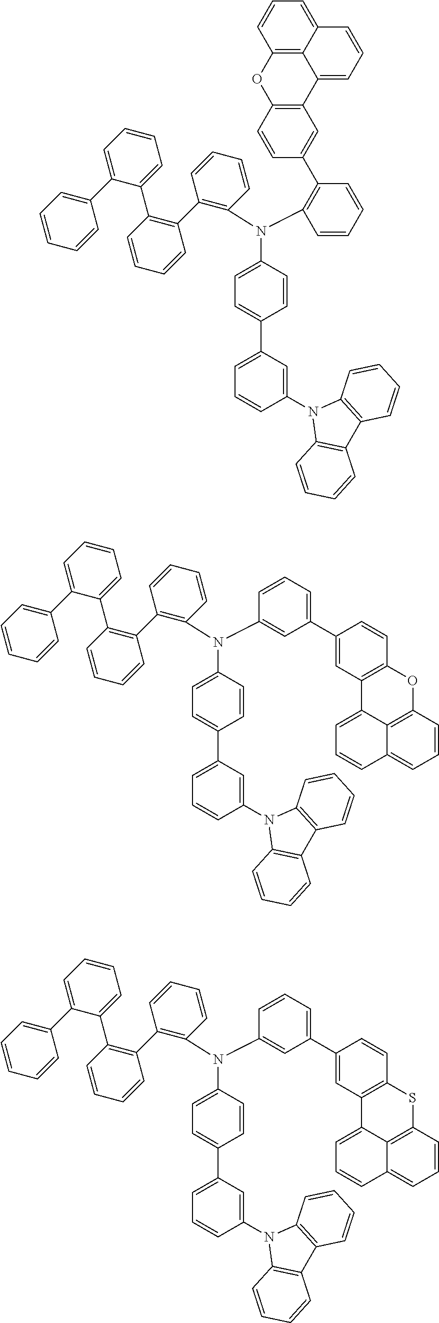 [SMILES: c1ccc(-c2ccccc2-c2ccccc2-c2ccccc2N(c2ccc(-c3cccc(-n4c5ccccc5c5ccccc54)c3)cc2)c2ccccc2-c2ccc3c(c2)-c2cccc4cccc(c24)O3)cc1 ; c1ccc(-c2ccccc2-c2ccccc2-c2ccccc2N(c2ccc(-c3cccc(-n4c5ccccc5c5ccccc54)c3)cc2)c2cccc(-c3ccc4c(c3)-c3cccc5cccc(c35)O4)c2)cc1 ; c1ccc(-c2ccccc2-c2ccccc2-c2ccccc2N(c2ccc(-c3cccc(-n4c5ccccc5c5ccccc54)c3)cc2)c2cccc(-c3ccc4c(c3)-c3cccc5cccc(c35)S4)c2)cc1]

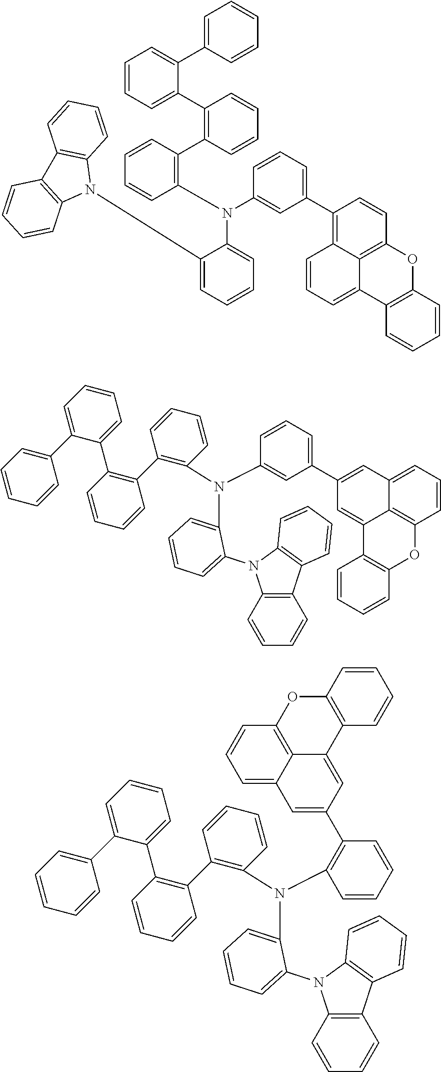 [SMILES: c1ccc(-c2ccccc2-c2ccccc2-c2ccccc2N(c2cccc(-c3ccc4c5c(cccc35)-c3ccccc3O4)c2)c2ccccc2-n2c3ccccc3c3ccccc32)cc1 ; c1ccc(-c2ccccc2-c2ccccc2-c2ccccc2N(c2cccc(-c3cc4c5c(cccc5c3)Oc3ccccc3-4)c2)c2ccccc2-n2c3ccccc3c3ccccc32)cc1 ; c1ccc(-c2ccccc2-c2ccccc2-c2ccccc2N(c2ccccc2-c2cc3c4c(cccc4c2)Oc2ccccc2-3)c2ccccc2-n2c3ccccc3c3ccccc32)cc1]

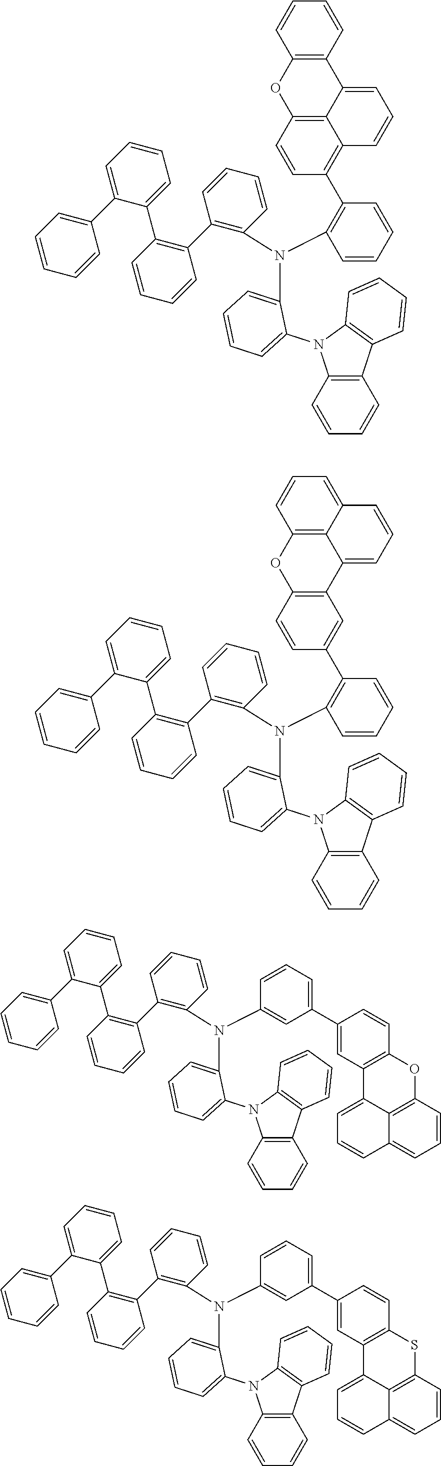

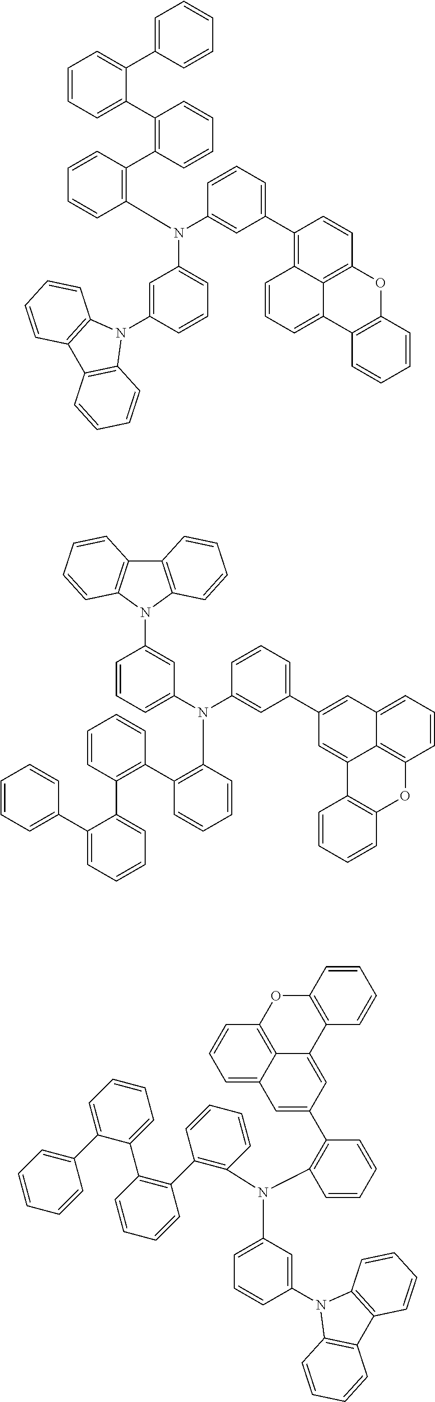 [SMILES: c1ccc(-c2ccccc2-c2ccccc2-c2ccccc2N(c2cccc(-c3ccc4c5c(cccc35)-c3ccccc3O4)c2)c2cccc(-n3c4ccccc4c4ccccc43)c2)cc1 ; c1ccc(-c2ccccc2-c2ccccc2-c2ccccc2N(c2cccc(-c3cc4c5c(cccc5c3)Oc3ccccc3-4)c2)c2cccc(-n3c4ccccc4c4ccccc43)c2)cc1 ; c1ccc(-c2ccccc2-c2ccccc2-c2ccccc2N(c2cccc(-n3c4ccccc4c4ccccc43)c2)c2ccccc2-c2cc3c4c(cccc4c2)Oc2ccccc2-3)cc1]

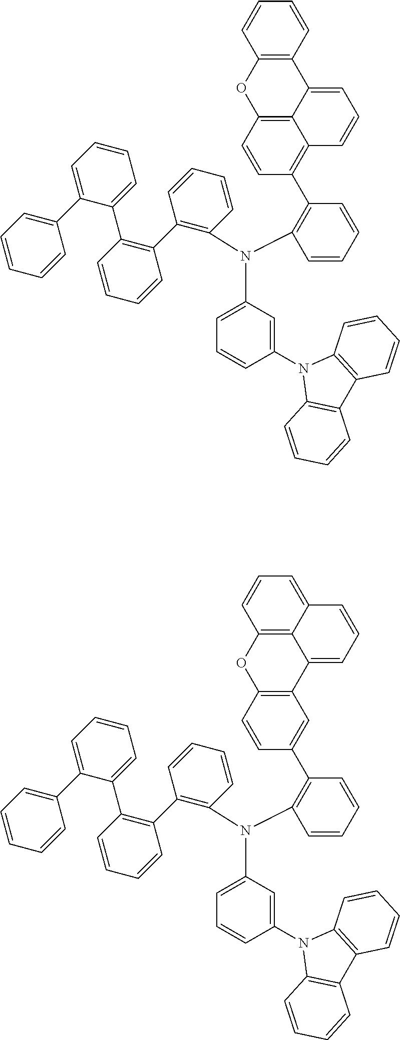 [SMILES: c1ccc(-c2ccccc2-c2ccccc2-c2ccccc2N(c2cccc(-n3c4ccccc4c4ccccc43)c2)c2ccccc2-c2ccc3c4c(cccc24)-c2ccccc2O3)cc1 ; c1ccc(-c2ccccc2-c2ccccc2-c2ccccc2N(c2cccc(-n3c4ccccc4c4ccccc43)c2)c2ccccc2-c2ccc3c(c2)-c2cccc4cccc(c24)O3)cc1]

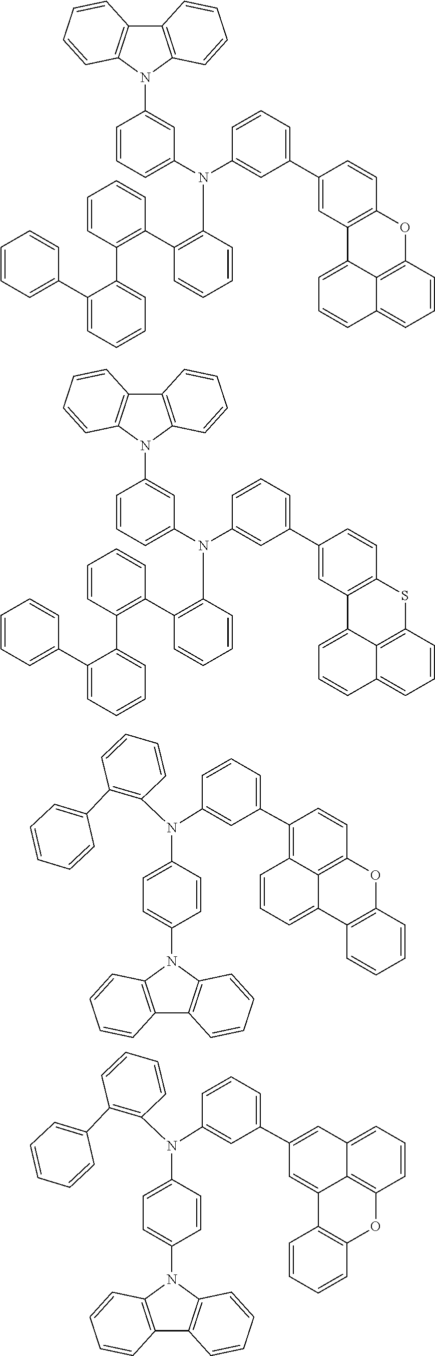

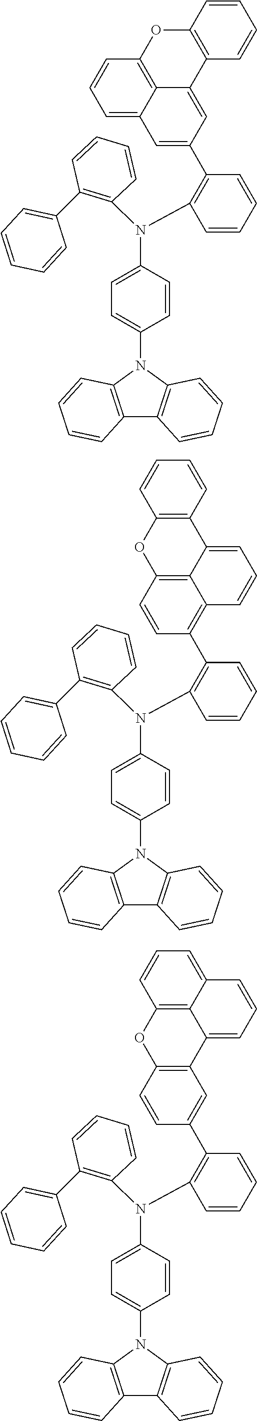

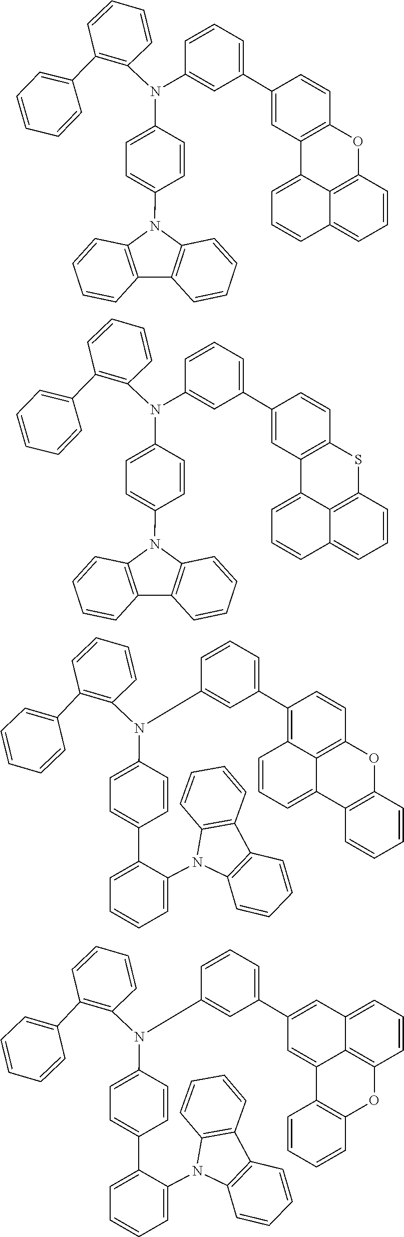

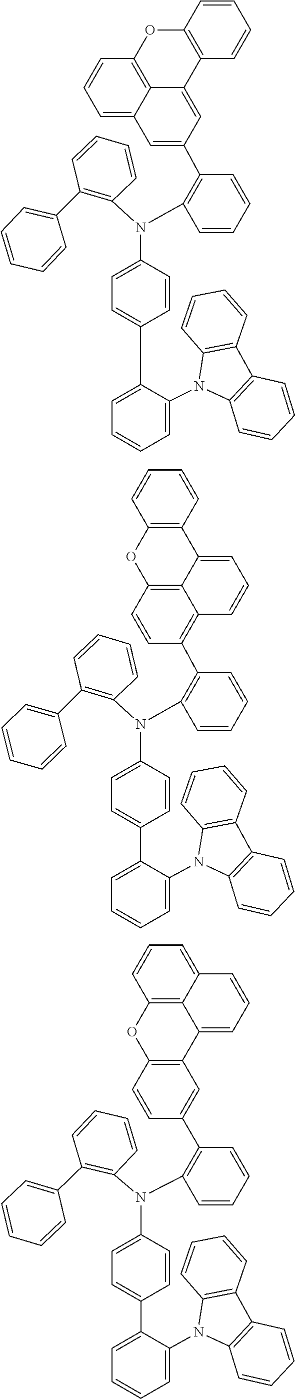

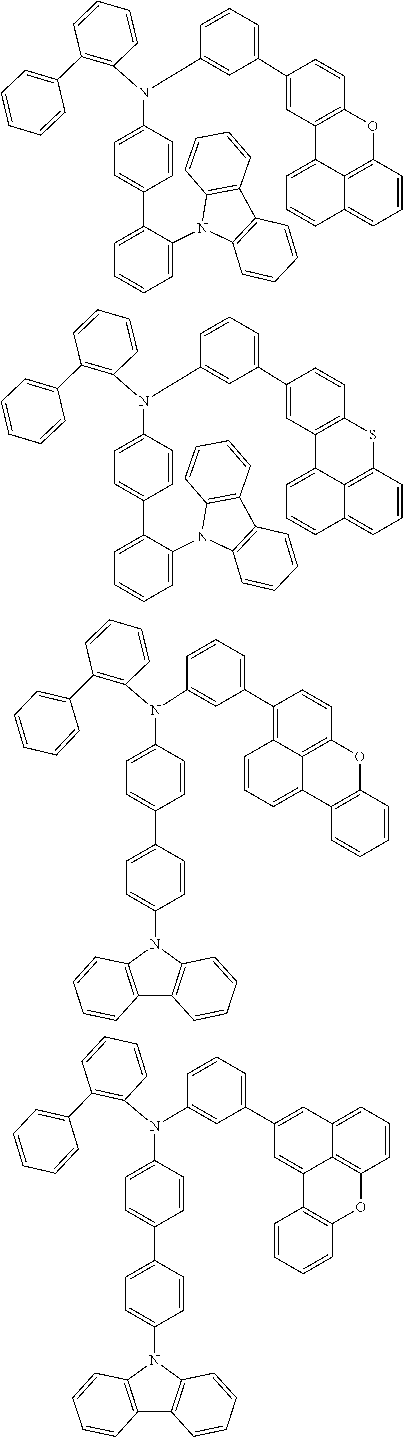

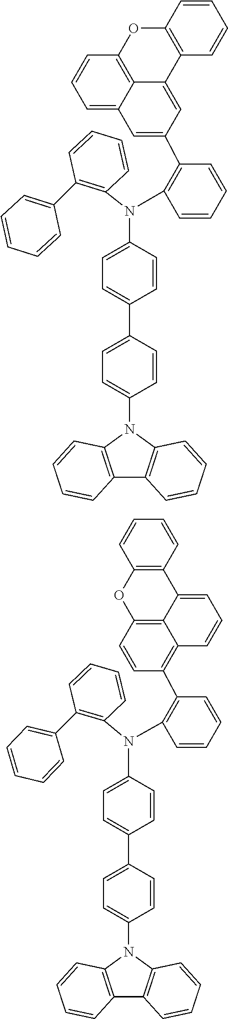

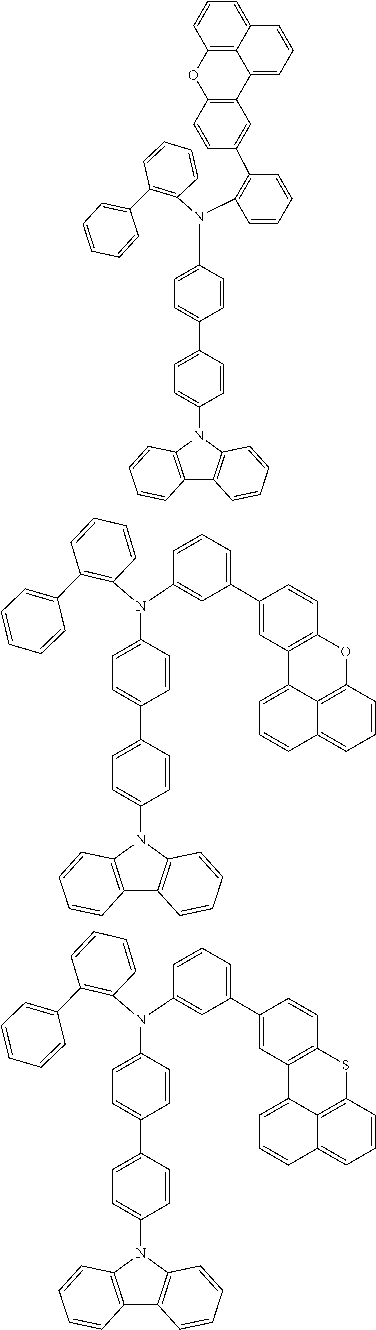

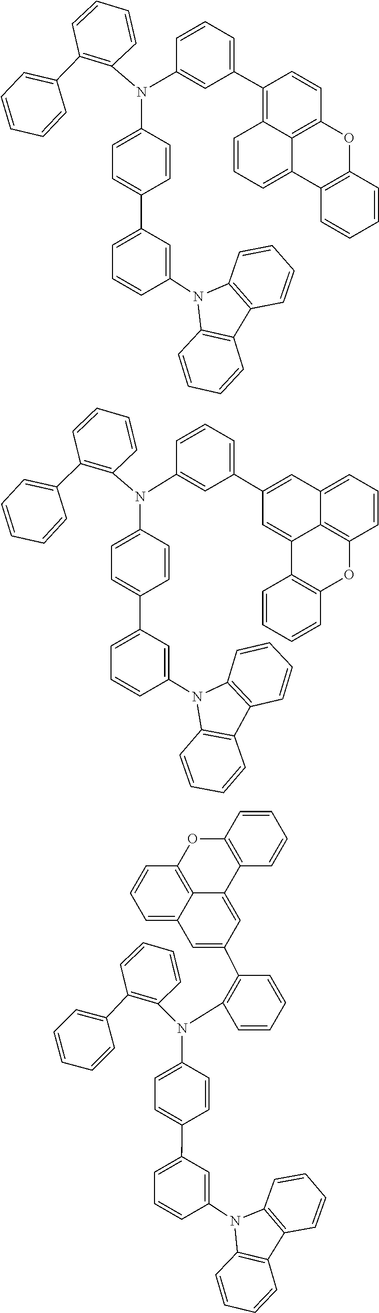

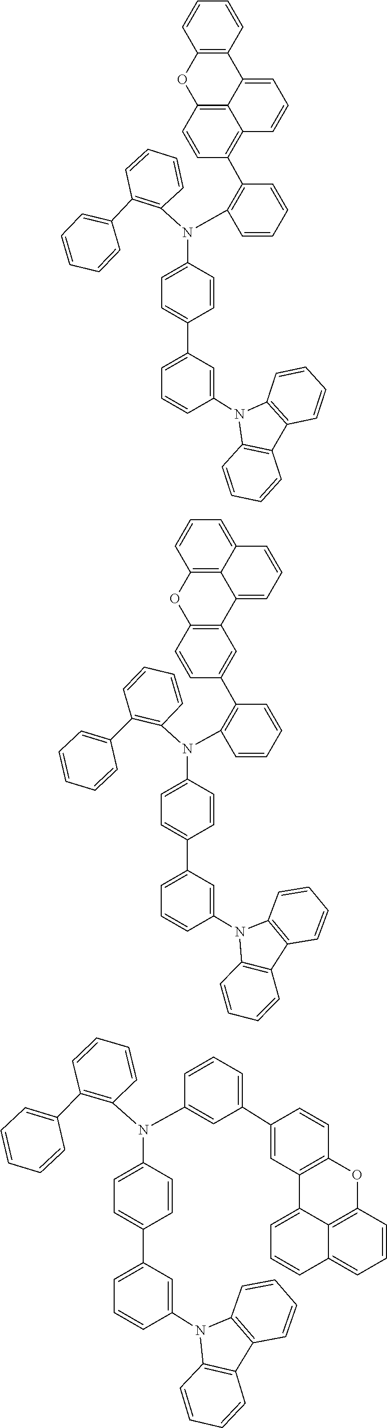

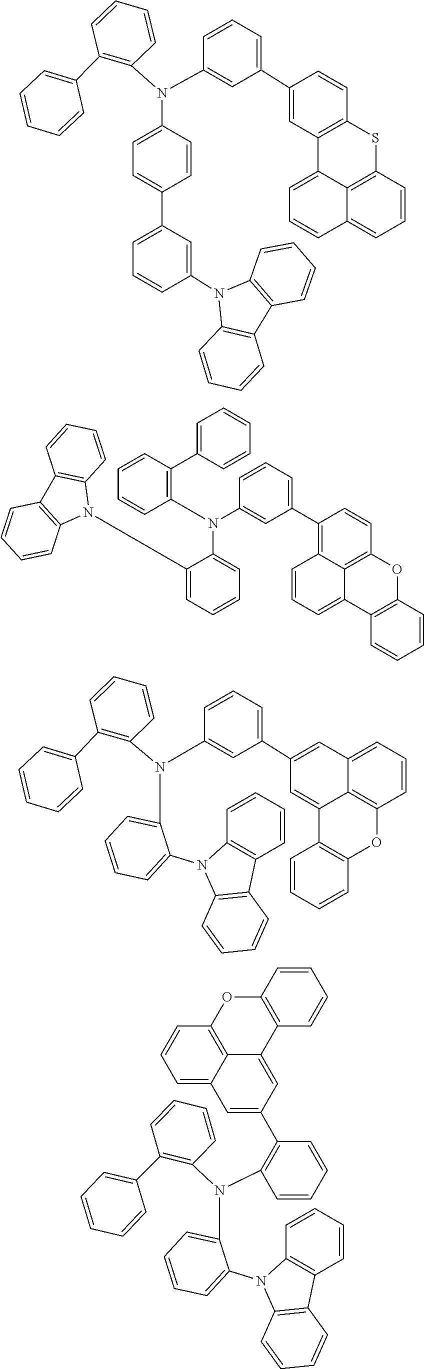

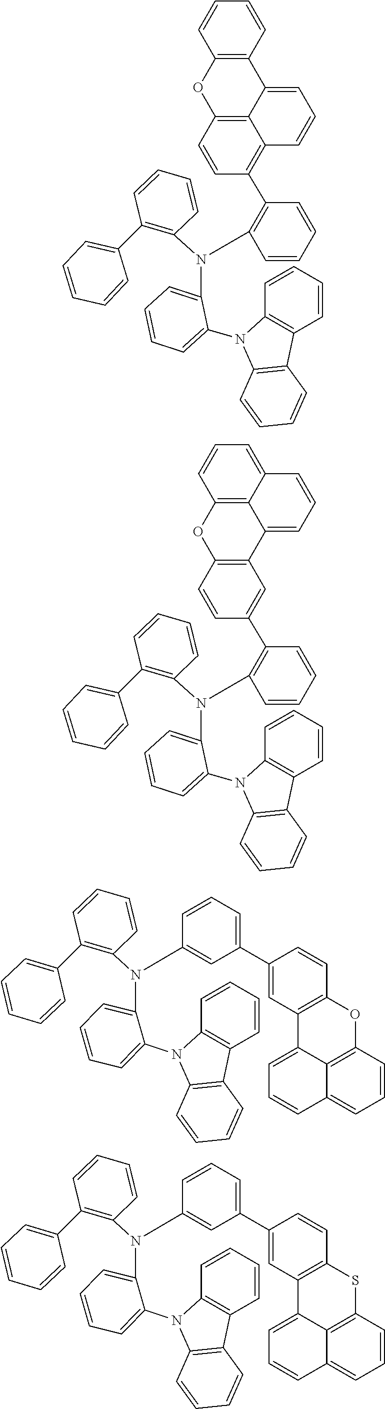

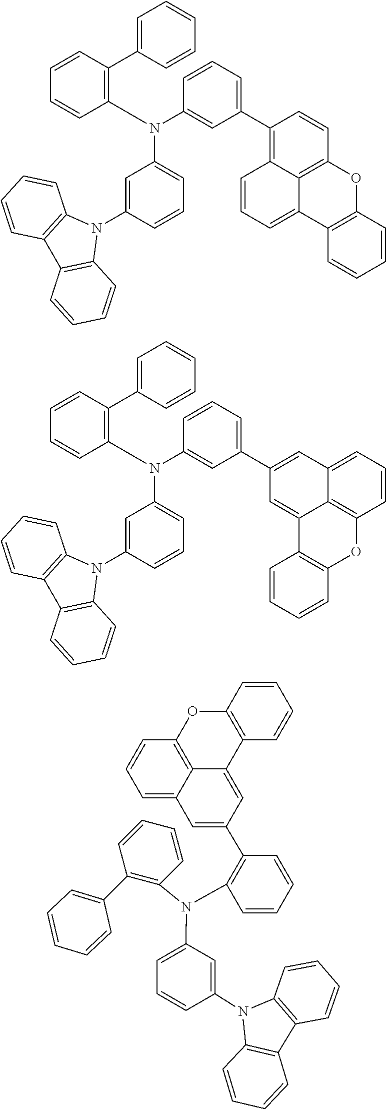 [SMILES: c1ccc(-c2ccccc2N(c2cccc(-c3ccc4c5c(cccc35)-c3ccccc3O4)c2)c2cccc(-n3c4ccccc4c4ccccc43)c2)cc1 ; c1ccc(-c2ccccc2N(c2cccc(-c3cc4c5c(cccc5c3)Oc3ccccc3-4)c2)c2cccc(-n3c4ccccc4c4ccccc43)c2)cc1 ; c1ccc(-c2ccccc2N(c2cccc(-n3c4ccccc4c4ccccc43)c2)c2ccccc2-c2cc3c4c(cccc4c2)Oc2ccccc2-3)cc1]

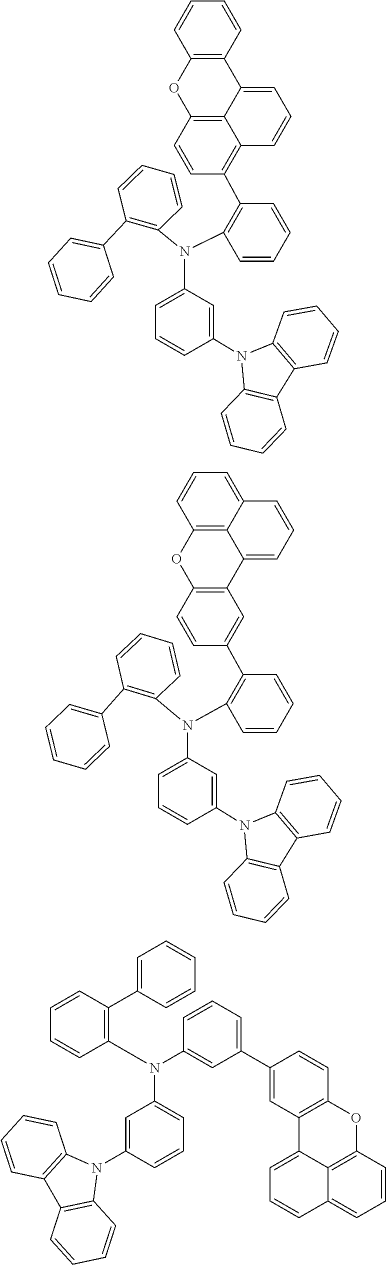

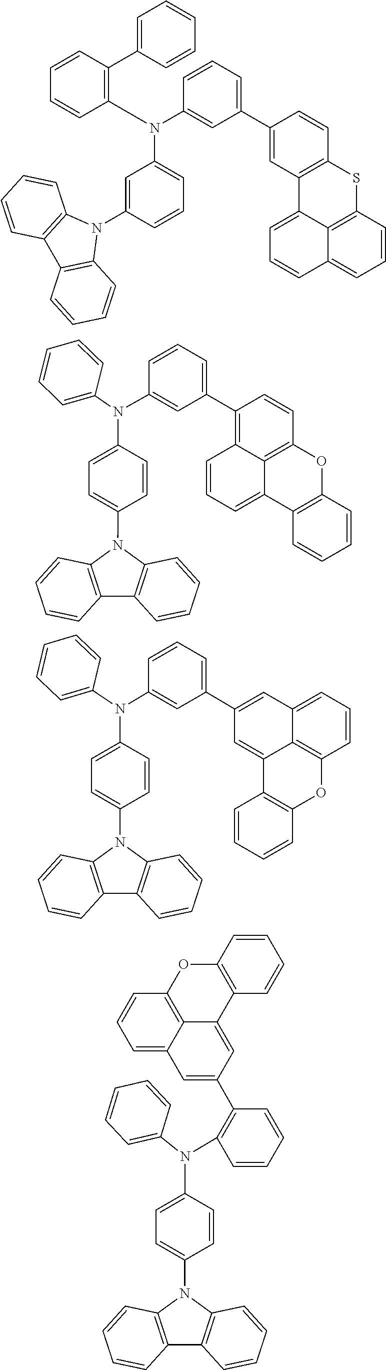

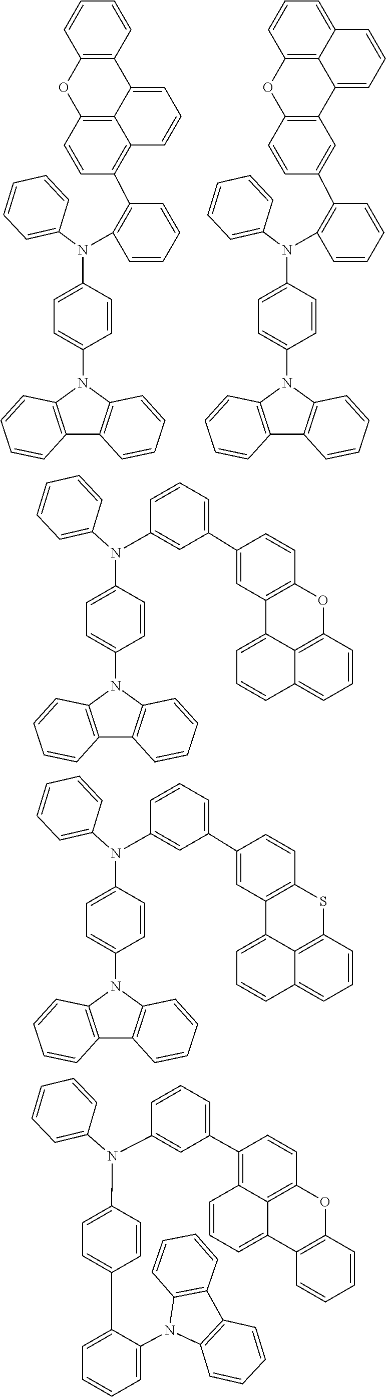

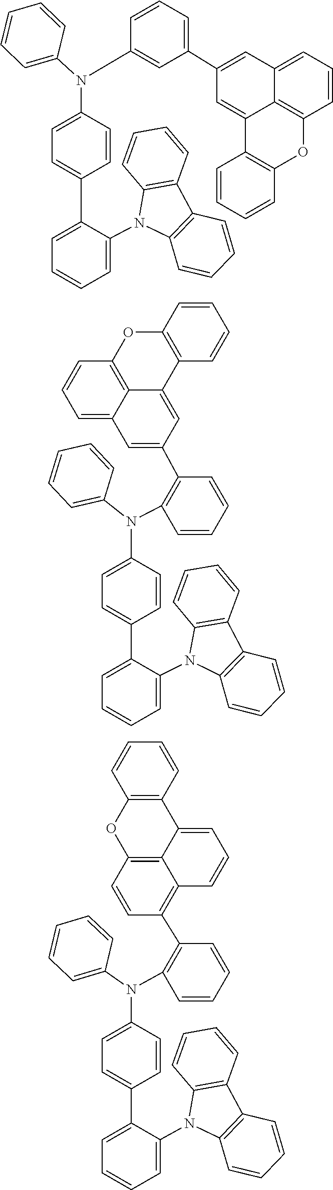

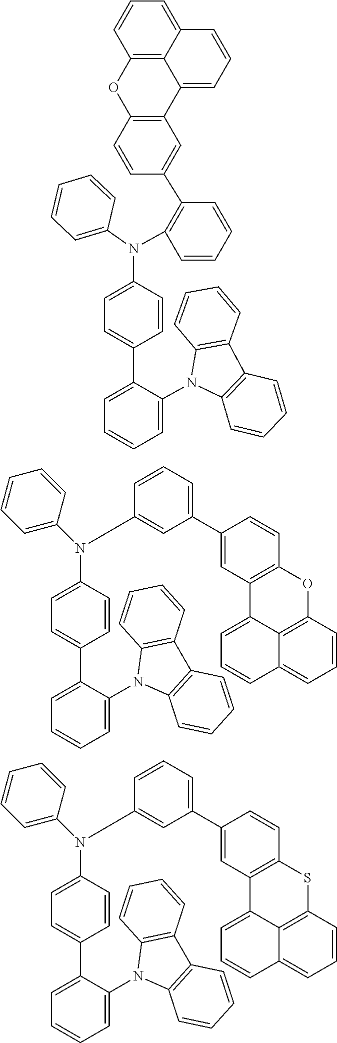

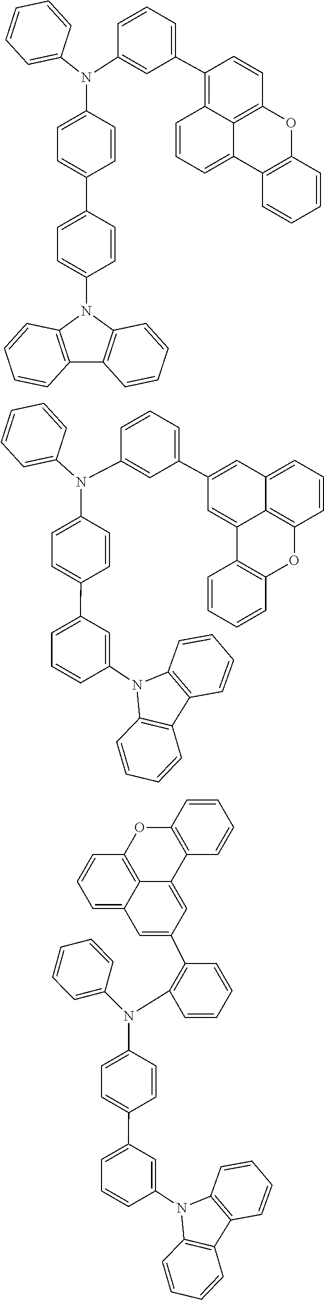

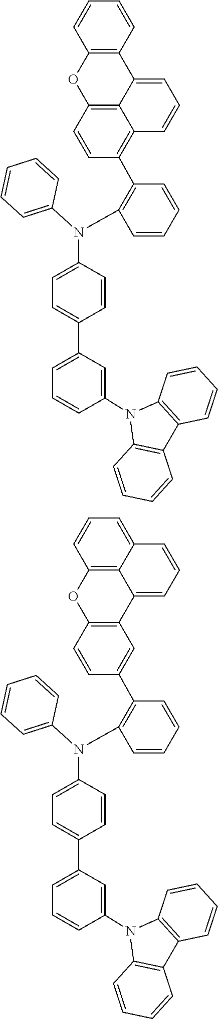

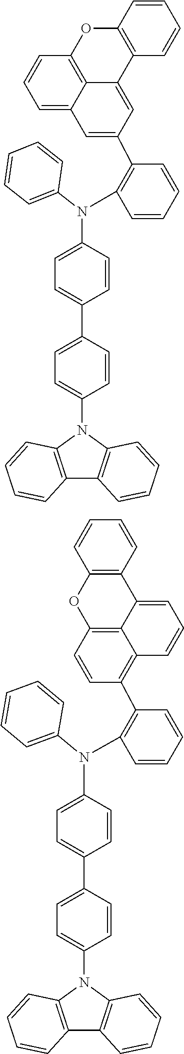

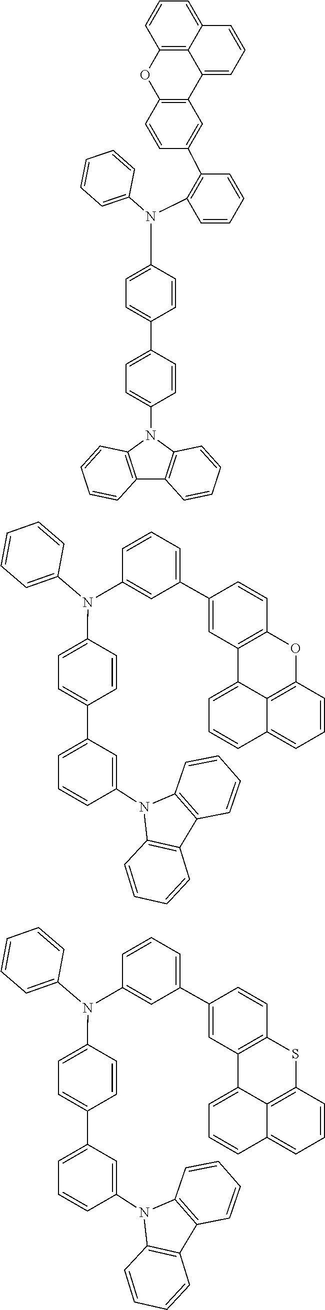

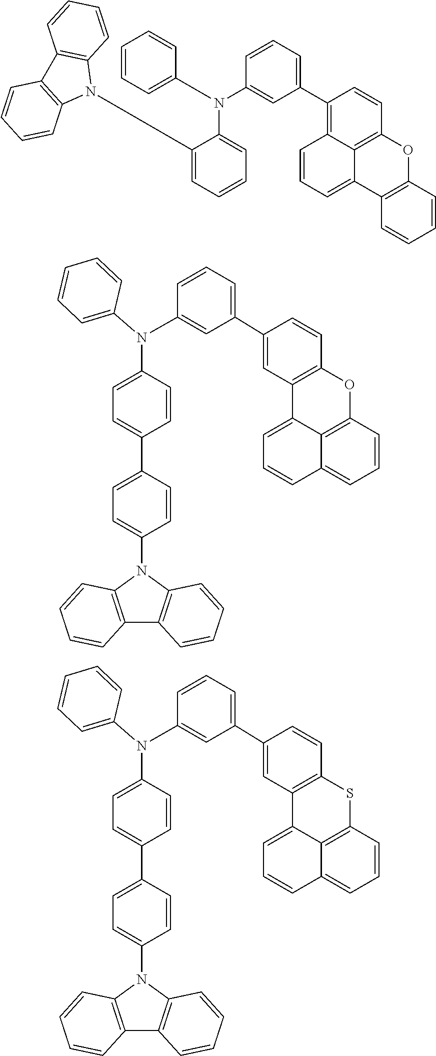

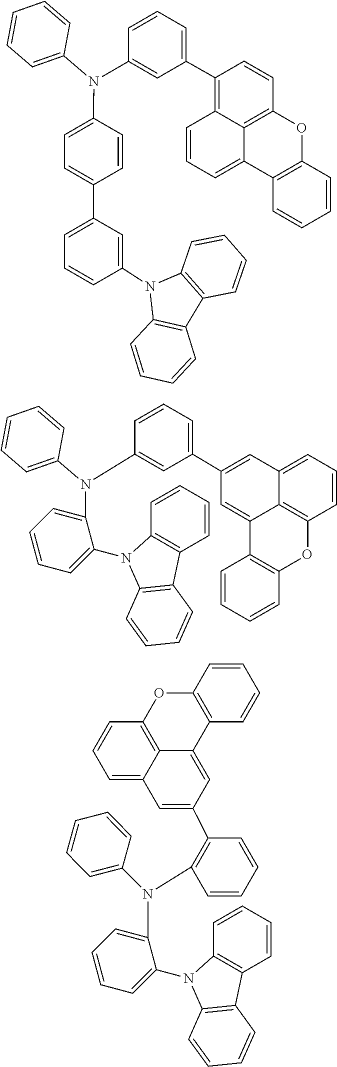

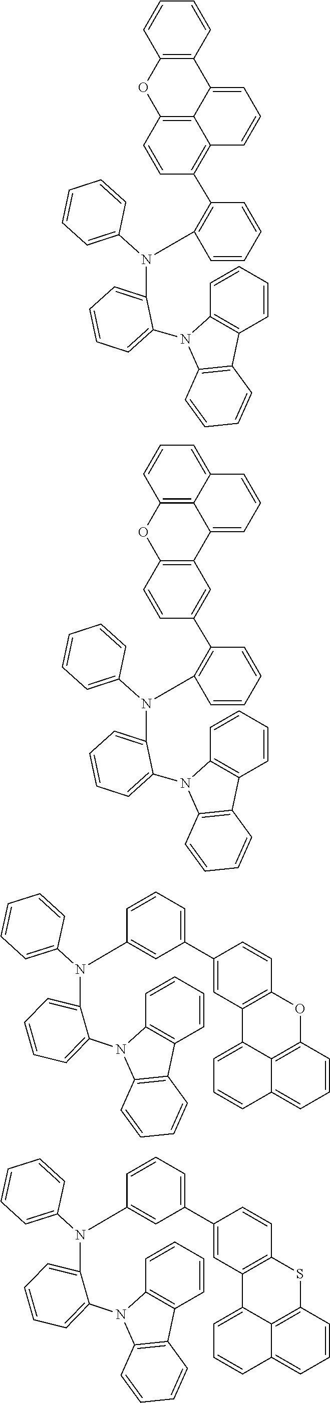

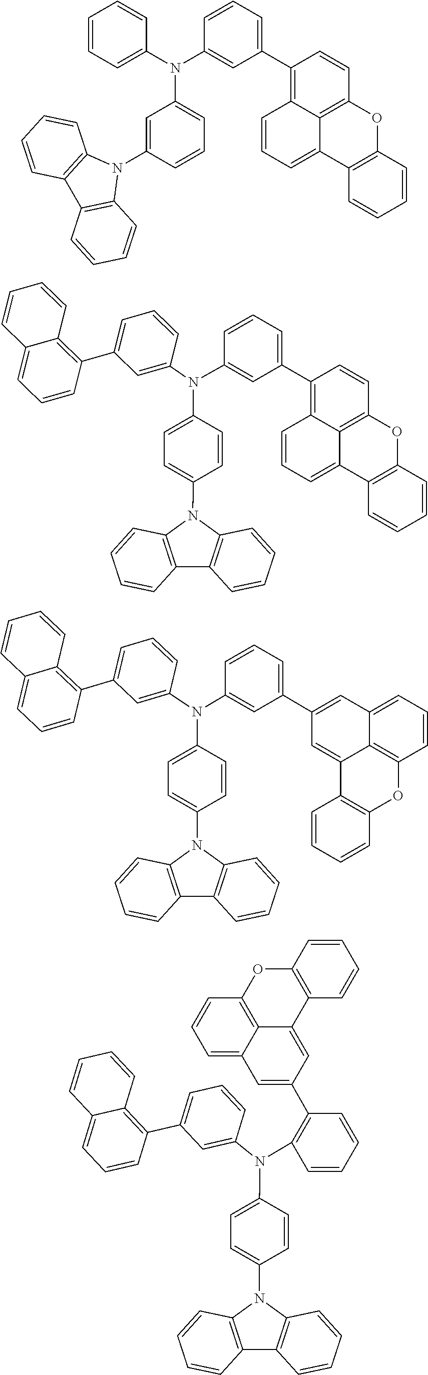

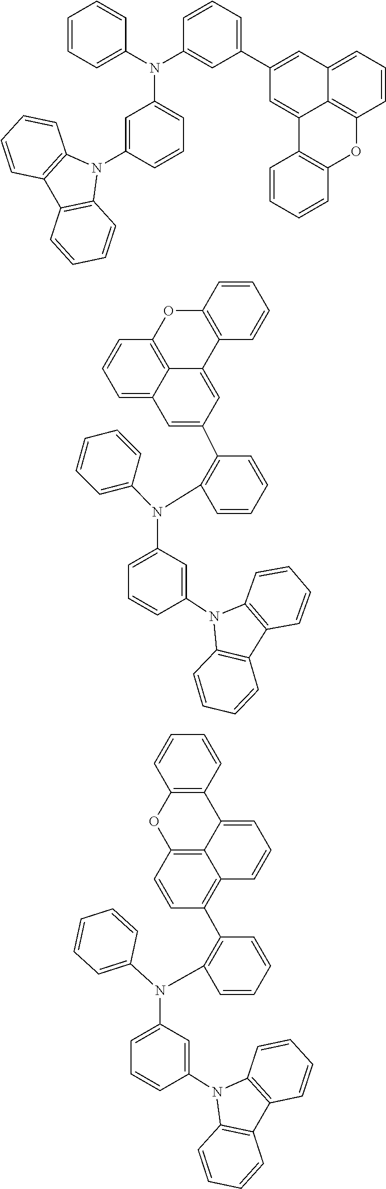

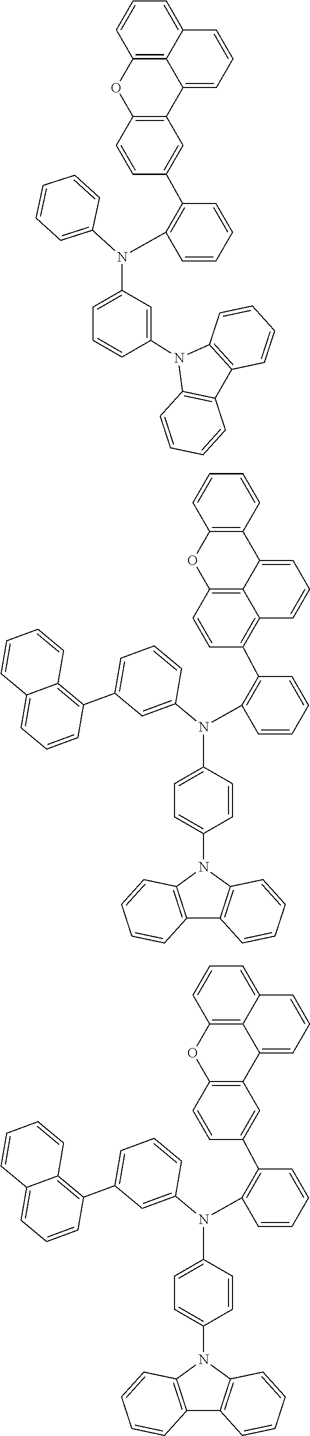

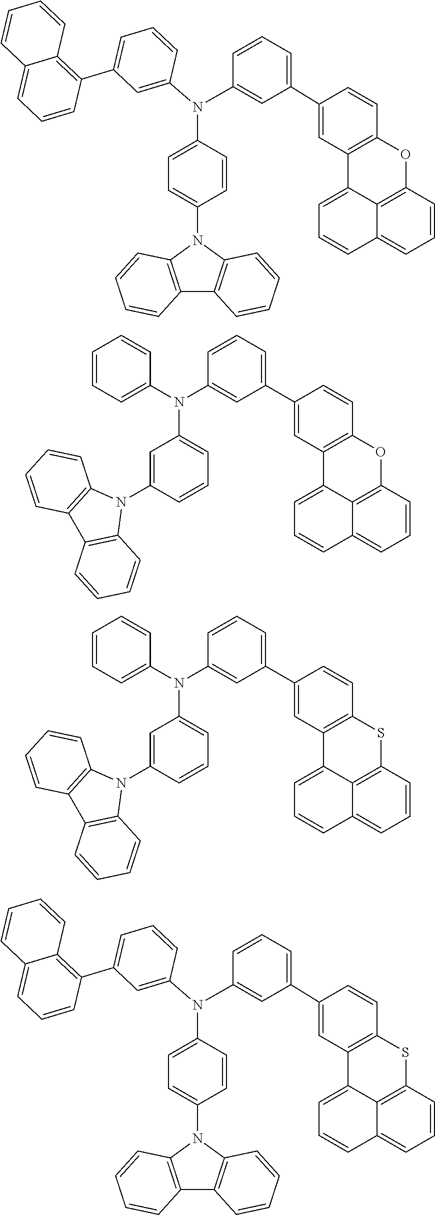

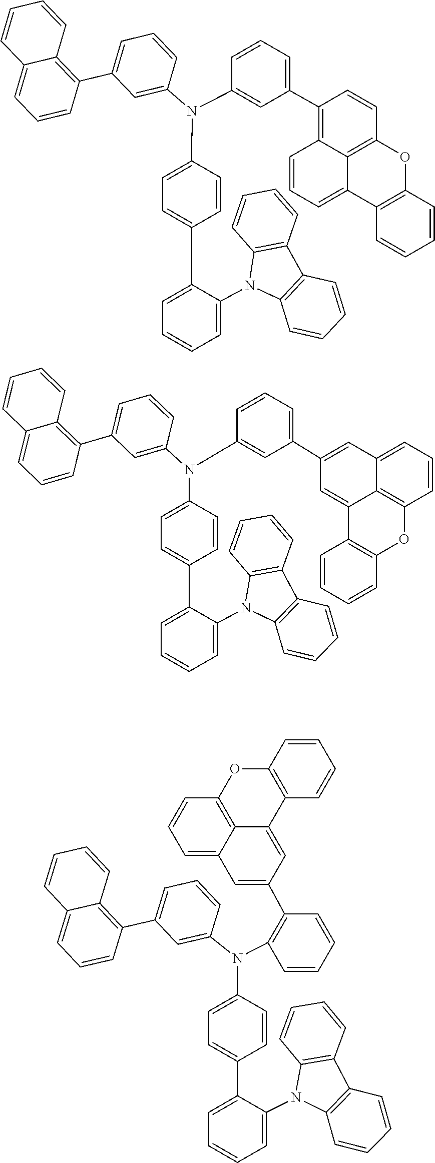 [SMILES: c1cc(-c2cccc3ccccc23)cc(N(c2ccc(-c3ccccc3-n3c4ccccc4c4ccccc43)cc2)c2cccc(-c3ccc4c5c(cccc35)-c3ccccc3O4)c2)c1 ; c1cc(-c2cc3c4c(cccc4c2)Oc2ccccc2-3)cc(N(c2ccc(-c3ccccc3-n3c4ccccc4c4ccccc43)cc2)c2cccc(-c3cccc4ccccc34)c2)c1 ; c1cc(-c2cccc3ccccc23)cc(N(c2ccc(-c3ccccc3-n3c4ccccc4c4ccccc43)cc2)c2ccccc2-c2cc3c4c(cccc4c2)Oc2ccccc2-3)c1]

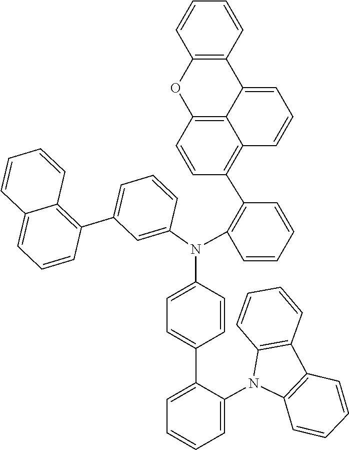 [SMILES: c1cc(-c2cccc3ccccc23)cc(N(c2ccc(-c3ccccc3-n3c4ccccc4c4ccccc43)cc2)c2ccccc2-c2ccc3c4c(cccc24)-c2ccccc2O3)c1]

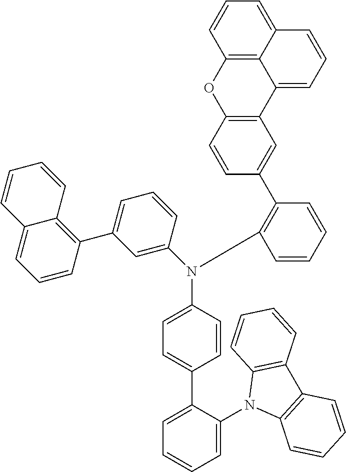 [SMILES: c1cc(-c2cccc3ccccc23)cc(N(c2ccc(-c3ccccc3-n3c4ccccc4c4ccccc43)cc2)c2ccccc2-c2ccc3c(c2)-c2cccc4cccc(c24)O3)c1]

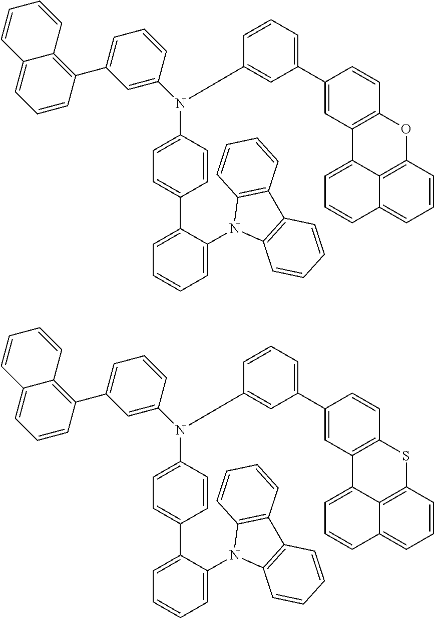 [SMILES: c1cc(-c2ccc3c(c2)-c2cccc4cccc(c24)O3)cc(N(c2ccc(-c3ccccc3-n3c4ccccc4c4ccccc43)cc2)c2cccc(-c3cccc4ccccc34)c2)c1 ; c1cc(-c2ccc3c(c2)-c2cccc4cccc(c24)S3)cc(N(c2ccc(-c3ccccc3-n3c4ccccc4c4ccccc43)cc2)c2cccc(-c3cccc4ccccc34)c2)c1]

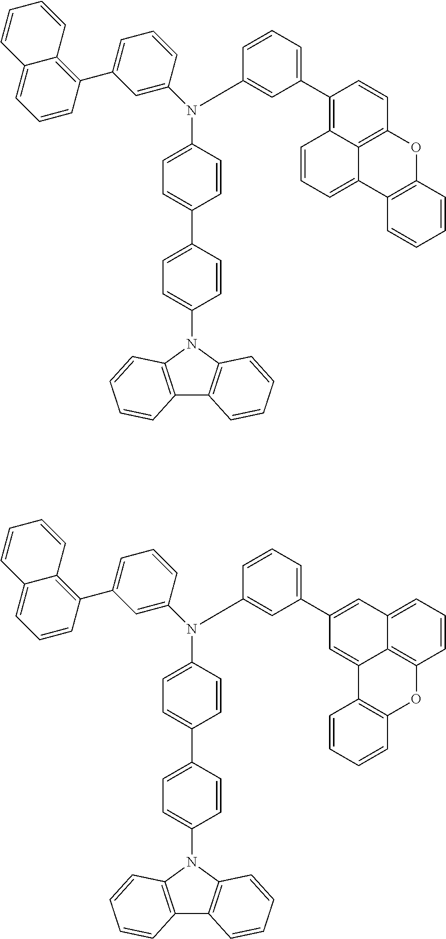 [SMILES: c1cc(-c2cccc3ccccc23)cc(N(c2ccc(-c3ccc(-n4c5ccccc5c5ccccc54)cc3)cc2)c2cccc(-c3ccc4c5c(cccc35)-c3ccccc3O4)c2)c1 ; c1cc(-c2cc3c4c(cccc4c2)Oc2ccccc2-3)cc(N(c2ccc(-c3ccc(-n4c5ccccc5c5ccccc54)cc3)cc2)c2cccc(-c3cccc4ccccc34)c2)c1]

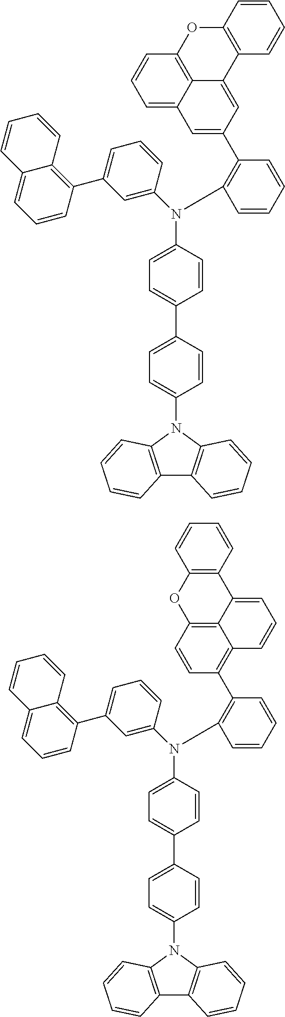

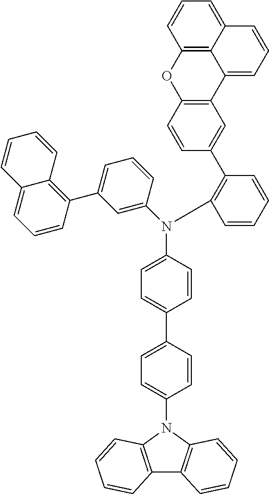 [SMILES: c1cc(-c2cccc3ccccc23)cc(N(c2ccc(-c3ccc(-n4c5ccccc5c5ccccc54)cc3)cc2)c2ccccc2-c2ccc3c(c2)-c2cccc4cccc(c24)O3)c1]

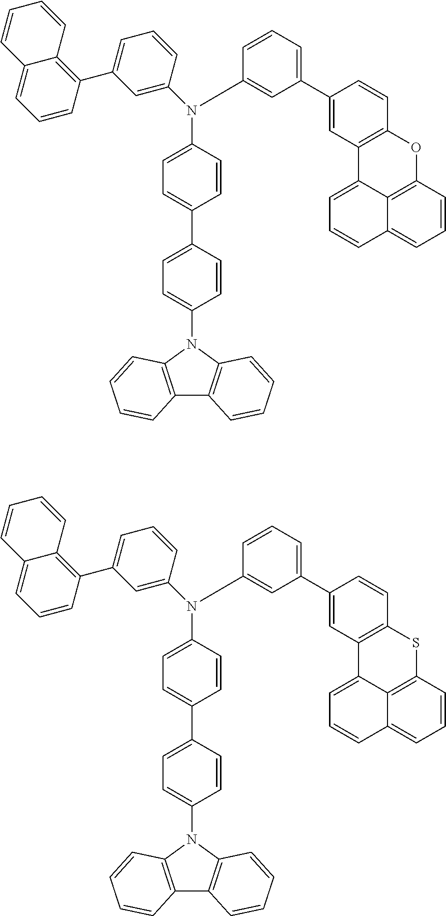 [SMILES: c1cc(-c2ccc3c(c2)-c2cccc4cccc(c24)O3)cc(N(c2ccc(-c3ccc(-n4c5ccccc5c5ccccc54)cc3)cc2)c2cccc(-c3cccc4ccccc34)c2)c1 ; c1cc(-c2ccc3c(c2)-c2cccc4cccc(c24)S3)cc(N(c2ccc(-c3ccc(-n4c5ccccc5c5ccccc54)cc3)cc2)c2cccc(-c3cccc4ccccc34)c2)c1]

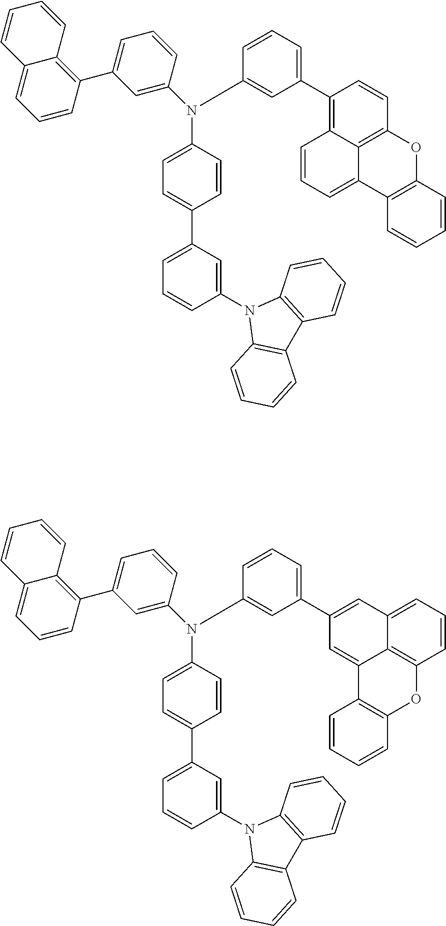 [SMILES: c1cc(-c2cccc3ccccc23)cc(N(c2ccc(-c3cccc(-n4c5ccccc5c5ccccc54)c3)cc2)c2cccc(-c3ccc4c5c(cccc35)-c3ccccc3O4)c2)c1 ; c1cc(-c2cc3c4c(cccc4c2)Oc2ccccc2-3)cc(N(c2ccc(-c3cccc(-n4c5ccccc5c5ccccc54)c3)cc2)c2cccc(-c3cccc4ccccc34)c2)c1]

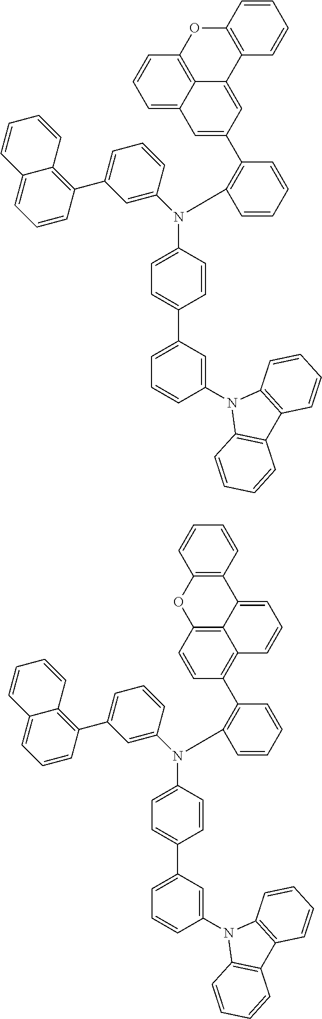

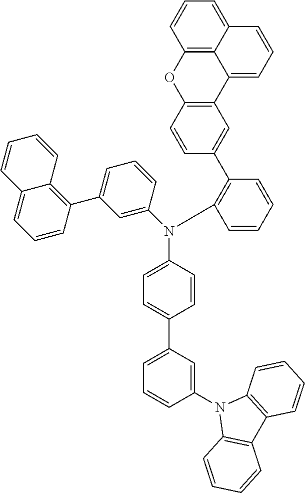 [SMILES: c1cc(-c2cccc3ccccc23)cc(N(c2ccc(-c3cccc(-n4c5ccccc5c5ccccc54)c3)cc2)c2ccccc2-c2ccc3c(c2)-c2cccc4cccc(c24)O3)c1]

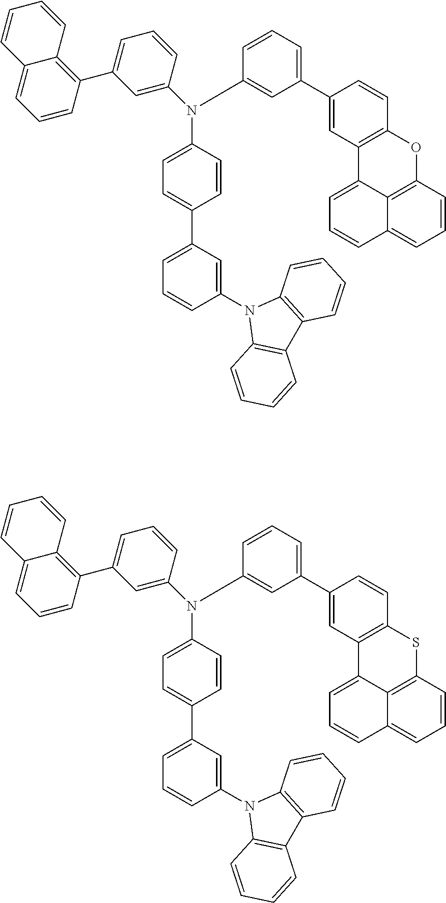 [SMILES: c1cc(-c2ccc3c(c2)-c2cccc4cccc(c24)O3)cc(N(c2ccc(-c3cccc(-n4c5ccccc5c5ccccc54)c3)cc2)c2cccc(-c3cccc4ccccc34)c2)c1 ; c1cc(-c2ccc3c(c2)-c2cccc4cccc(c24)S3)cc(N(c2ccc(-c3cccc(-n4c5ccccc5c5ccccc54)c3)cc2)c2cccc(-c3cccc4ccccc34)c2)c1]

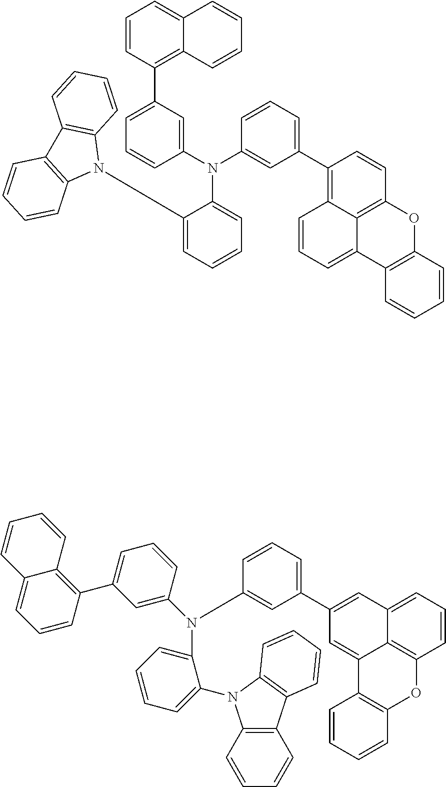 [SMILES: c1cc(-c2cccc3ccccc23)cc(N(c2cccc(-c3ccc4c5c(cccc35)-c3ccccc3O4)c2)c2ccccc2-n2c3ccccc3c3ccccc32)c1 ; c1cc(-c2cc3c4c(cccc4c2)Oc2ccccc2-3)cc(N(c2cccc(-c3cccc4ccccc34)c2)c2ccccc2-n2c3ccccc3c3ccccc32)c1]

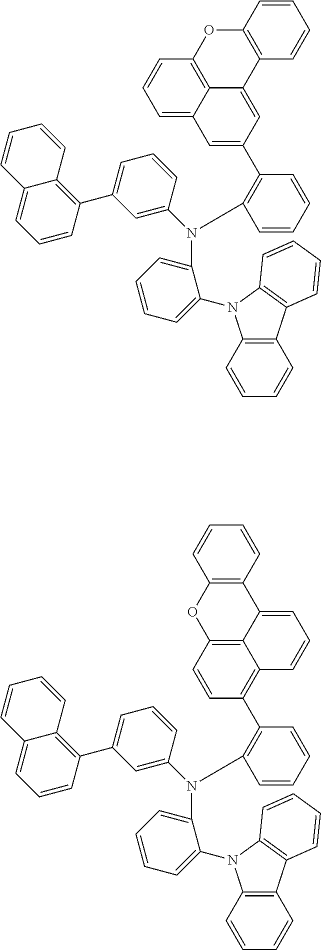 [SMILES: c1cc(-c2cccc3ccccc23)cc(N(c2ccccc2-c2cc3c4c(cccc4c2)Oc2ccccc2-3)c2ccccc2-n2c3ccccc3c3ccccc32)c1 ; c1cc(-c2cccc3ccccc23)cc(N(c2ccccc2-c2ccc3c4c(cccc24)-c2ccccc2O3)c2ccccc2-n2c3ccccc3c3ccccc32)c1]

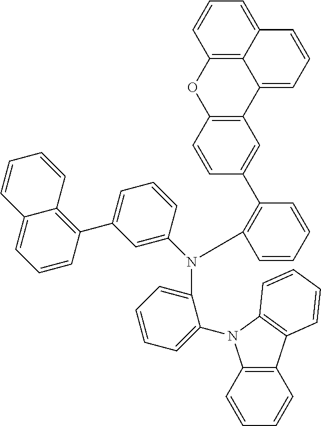 [SMILES: c1cc(-c2cccc3ccccc23)cc(N(c2ccccc2-c2ccc3c(c2)-c2cccc4cccc(c24)O3)c2ccccc2-n2c3ccccc3c3ccccc32)c1]

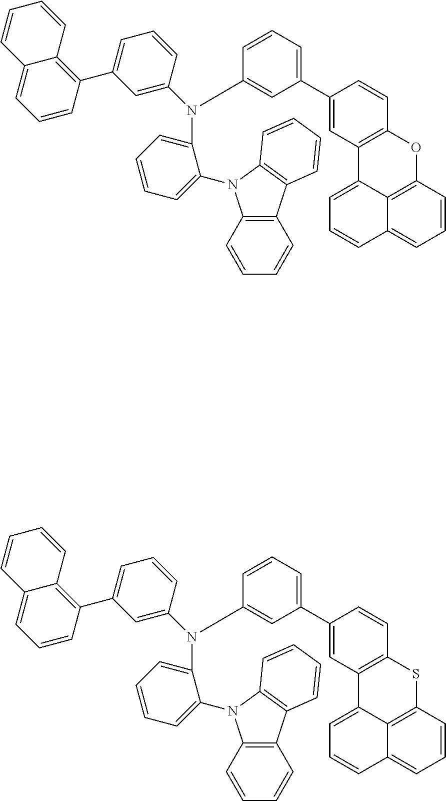 [SMILES: c1cc(-c2ccc3c(c2)-c2cccc4cccc(c24)O3)cc(N(c2cccc(-c3cccc4ccccc34)c2)c2ccccc2-n2c3ccccc3c3ccccc32)c1 ; c1cc(-c2ccc3c(c2)-c2cccc4cccc(c24)S3)cc(N(c2cccc(-c3cccc4ccccc34)c2)c2ccccc2-n2c3ccccc3c3ccccc32)c1]

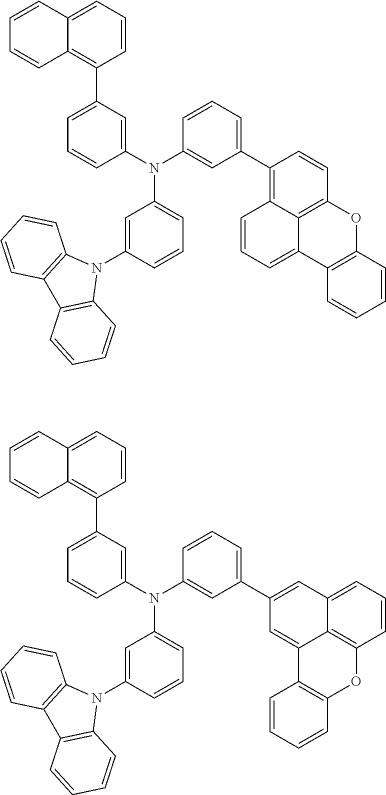 [SMILES: c1cc(-c2cccc3ccccc23)cc(N(c2cccc(-c3ccc4c5c(cccc35)-c3ccccc3O4)c2)c2cccc(-n3c4ccccc4c4ccccc43)c2)c1 ; c1cc(-c2cc3c4c(cccc4c2)Oc2ccccc2-3)cc(N(c2cccc(-c3cccc4ccccc34)c2)c2cccc(-n3c4ccccc4c4ccccc43)c2)c1]

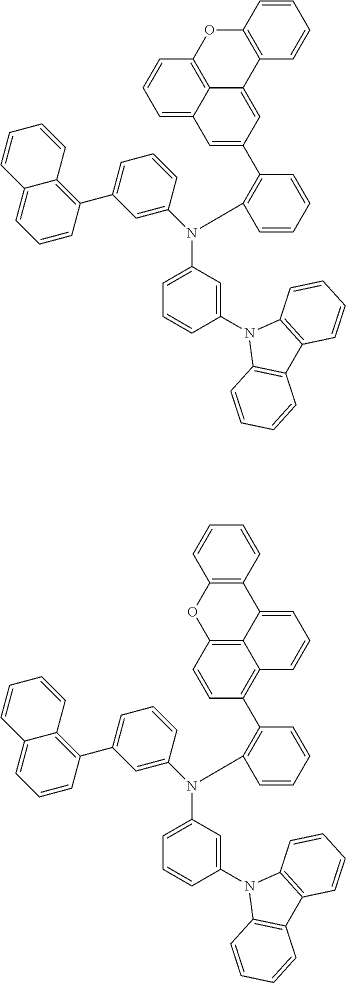 [SMILES: c1cc(-c2cccc3ccccc23)cc(N(c2cccc(-n3c4ccccc4c4ccccc43)c2)c2ccccc2-c2cc3c4c(cccc4c2)Oc2ccccc2-3)c1 ; c1cc(-c2cccc3ccccc23)cc(N(c2cccc(-n3c4ccccc4c4ccccc43)c2)c2ccccc2-c2ccc3c4c(cccc24)-c2ccccc2O3)c1]

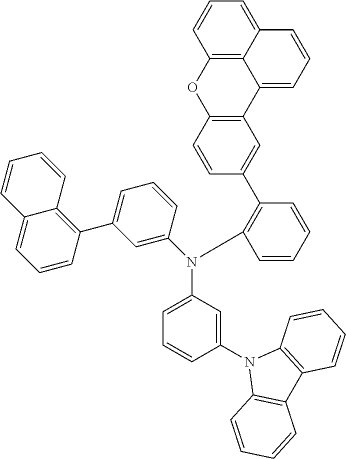 [SMILES: c1cc(-c2cccc3ccccc23)cc(N(c2cccc(-n3c4ccccc4c4ccccc43)c2)c2ccccc2-c2ccc3c(c2)-c2cccc4cccc(c24)O3)c1]

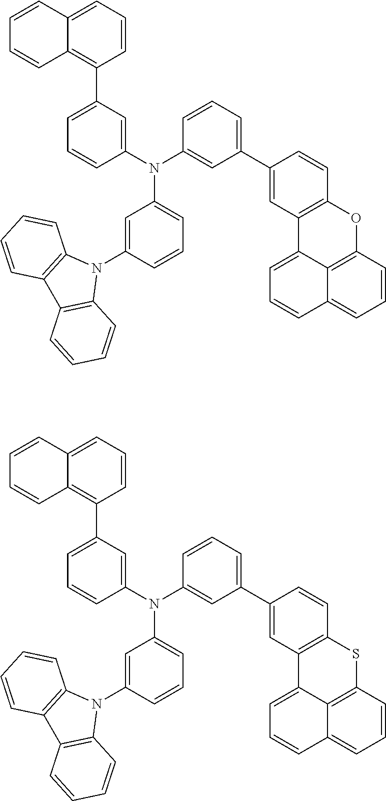 [SMILES: c1cc(-c2ccc3c(c2)-c2cccc4cccc(c24)O3)cc(N(c2cccc(-c3cccc4ccccc34)c2)c2cccc(-n3c4ccccc4c4ccccc43)c2)c1 ; c1cc(-c2ccc3c(c2)-c2cccc4cccc(c24)S3)cc(N(c2cccc(-c3cccc4ccccc34)c2)c2cccc(-n3c4ccccc4c4ccccc43)c2)c1]

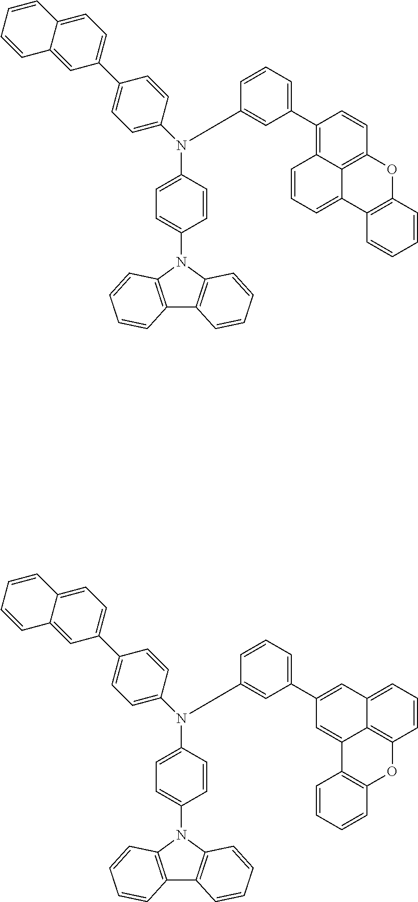 [SMILES: c1cc(-c2ccc3c4c(cccc24)-c2ccccc2O3)cc(N(c2ccc(-c3ccc4ccccc4c3)cc2)c2ccc(-n3c4ccccc4c4ccccc43)cc2)c1 ; c1cc(-c2cc3c4c(cccc4c2)Oc2ccccc2-3)cc(N(c2ccc(-c3ccc4ccccc4c3)cc2)c2ccc(-n3c4ccccc4c4ccccc43)cc2)c1]

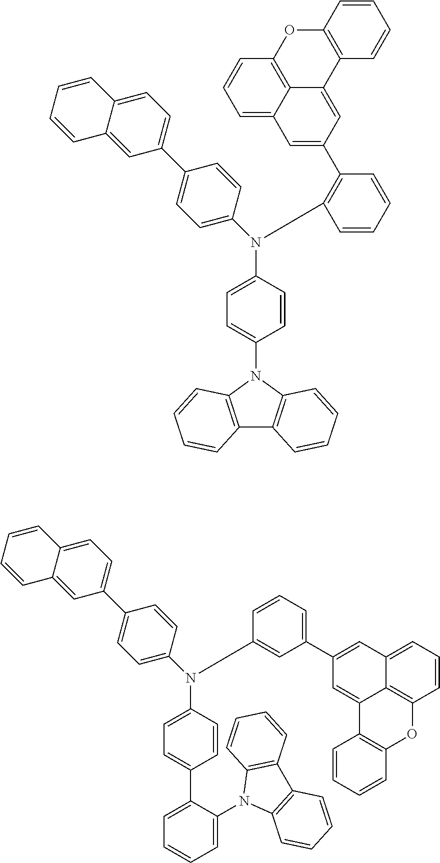 [SMILES: c1ccc2c(c1)Oc1cccc3cc(-c4ccccc4N(c4ccc(-c5ccc6ccccc6c5)cc4)c4ccc(-n5c6ccccc6c6ccccc65)cc4)cc-2c13 ; c1cc(-c2cc3c4c(cccc4c2)Oc2ccccc2-3)cc(N(c2ccc(-c3ccc4ccccc4c3)cc2)c2ccc(-c3ccccc3-n3c4ccccc4c4ccccc43)cc2)c1]

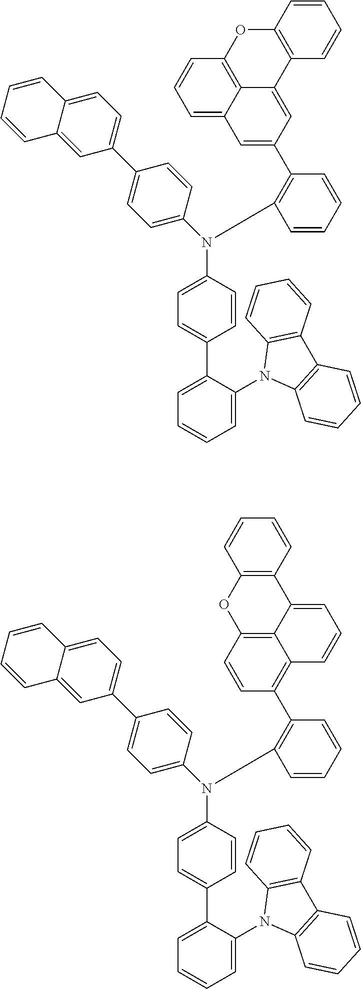 [SMILES: c1ccc2c(c1)Oc1cccc3cc(-c4ccccc4N(c4ccc(-c5ccc6ccccc6c5)cc4)c4ccc(-c5ccccc5-n5c6ccccc6c6ccccc65)cc4)cc-2c13 ; c1ccc2c(c1)Oc1ccc(-c3ccccc3N(c3ccc(-c4ccc5ccccc5c4)cc3)c3ccc(-c4ccccc4-n4c5ccccc5c5ccccc54)cc3)c3cccc-2c13]

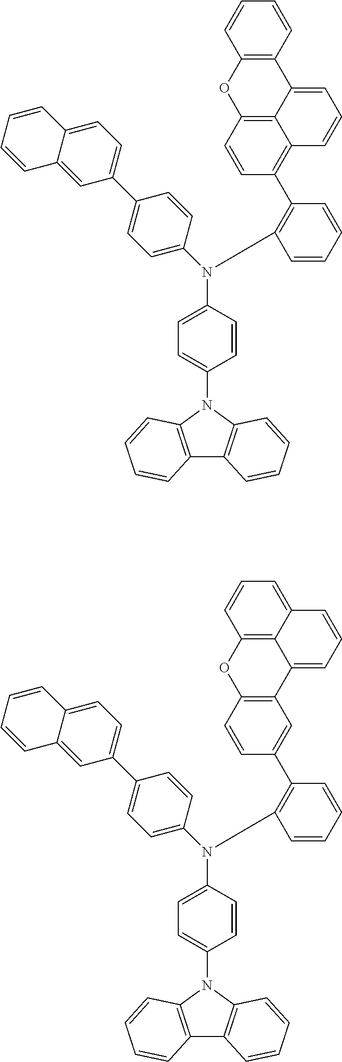 [SMILES: c1ccc2c(c1)Oc1ccc(-c3ccccc3N(c3ccc(-c4ccc5ccccc5c4)cc3)c3ccc(-n4c5ccccc5c5ccccc54)cc3)c3cccc-2c13 ; c1ccc(N(c2ccc(-c3ccc4ccccc4c3)cc2)c2ccc(-n3c4ccccc4c4ccccc43)cc2)c(-c2ccc3c(c2)-c2cccc4cccc(c24)O3)c1]

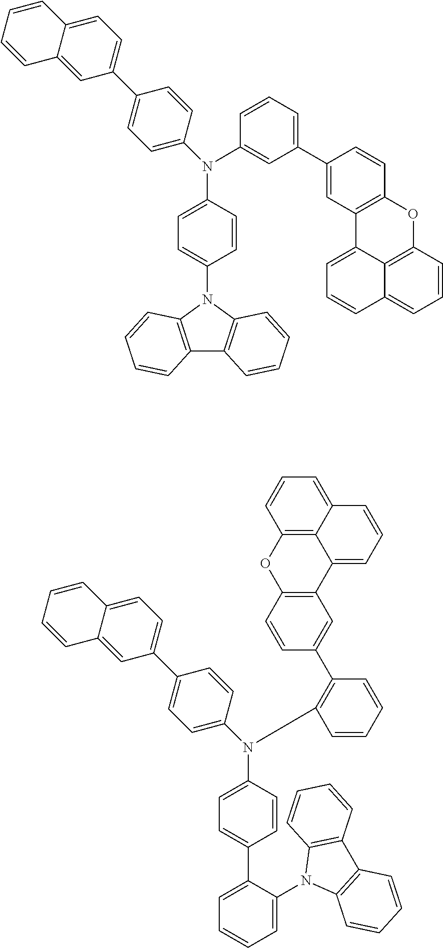 [SMILES: c1cc(-c2ccc3c(c2)-c2cccc4cccc(c24)O3)cc(N(c2ccc(-c3ccc4ccccc4c3)cc2)c2ccc(-n3c4ccccc4c4ccccc43)cc2)c1 ; c1ccc(N(c2ccc(-c3ccc4ccccc4c3)cc2)c2ccc(-c3ccccc3-n3c4ccccc4c4ccccc43)cc2)c(-c2ccc3c(c2)-c2cccc4cccc(c24)O3)c1]

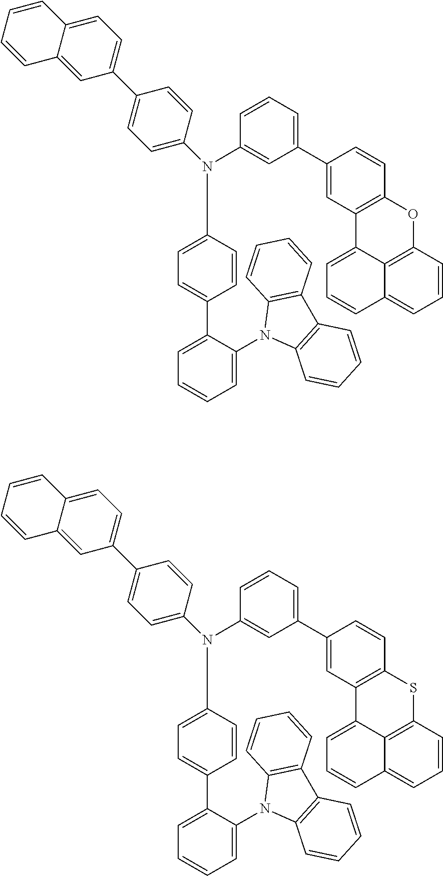 [SMILES: c1cc(-c2ccc3c(c2)-c2cccc4cccc(c24)O3)cc(N(c2ccc(-c3ccc4ccccc4c3)cc2)c2ccc(-c3ccccc3-n3c4ccccc4c4ccccc43)cc2)c1 ; c1cc(-c2ccc3c(c2)-c2cccc4cccc(c24)S3)cc(N(c2ccc(-c3ccc4ccccc4c3)cc2)c2ccc(-c3ccccc3-n3c4ccccc4c4ccccc43)cc2)c1]

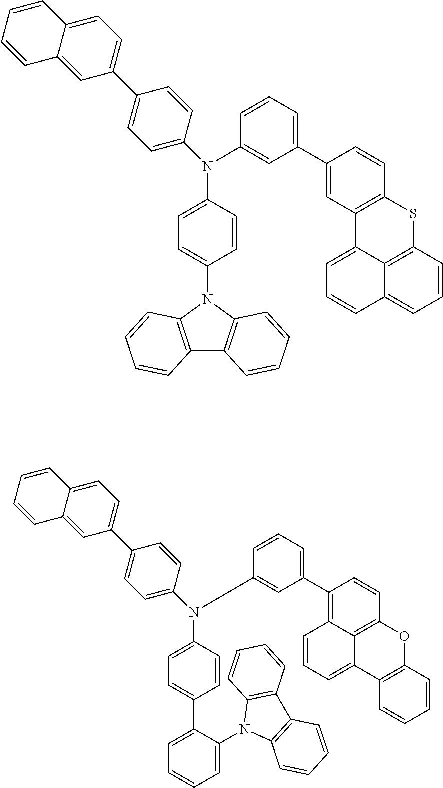 [SMILES: c1cc(-c2ccc3c(c2)-c2cccc4cccc(c24)S3)cc(N(c2ccc(-c3ccc4ccccc4c3)cc2)c2ccc(-n3c4ccccc4c4ccccc43)cc2)c1 ; c1cc(-c2ccc3c4c(cccc24)-c2ccccc2O3)cc(N(c2ccc(-c3ccc4ccccc4c3)cc2)c2ccc(-c3ccccc3-n3c4ccccc4c4ccccc43)cc2)c1]

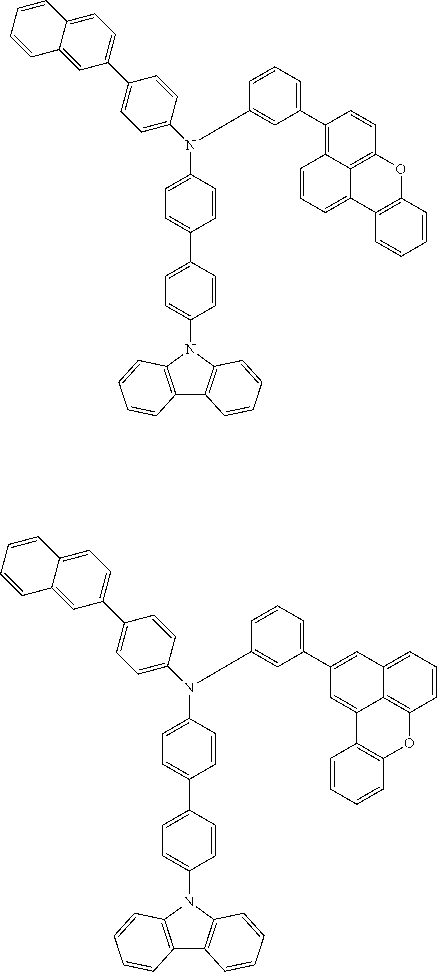 [SMILES: c1cc(-c2ccc3c4c(cccc24)-c2ccccc2O3)cc(N(c2ccc(-c3ccc(-n4c5ccccc5c5ccccc54)cc3)cc2)c2ccc(-c3ccc4ccccc4c3)cc2)c1 ; c1cc(-c2cc3c4c(cccc4c2)Oc2ccccc2-3)cc(N(c2ccc(-c3ccc(-n4c5ccccc5c5ccccc54)cc3)cc2)c2ccc(-c3ccc4ccccc4c3)cc2)c1]

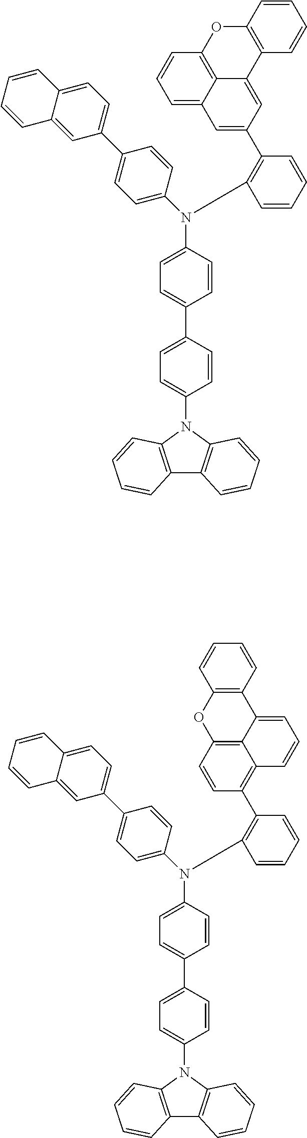 [SMILES: c1ccc2c(c1)Oc1cccc3cc(-c4ccccc4N(c4ccc(-c5ccc(-n6c7ccccc7c7ccccc76)cc5)cc4)c4ccc(-c5ccc6ccccc6c5)cc4)cc-2c13 ; c1ccc2c(c1)Oc1ccc(-c3ccccc3N(c3ccc(-c4ccc(-n5c6ccccc6c6ccccc65)cc4)cc3)c3ccc(-c4ccc5ccccc5c4)cc3)c3cccc-2c13]

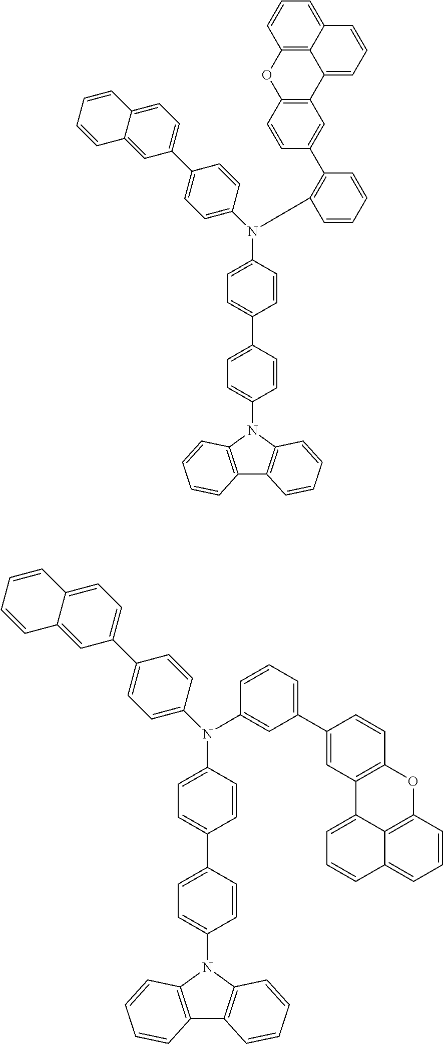 [SMILES: c1ccc(N(c2ccc(-c3ccc(-n4c5ccccc5c5ccccc54)cc3)cc2)c2ccc(-c3ccc4ccccc4c3)cc2)c(-c2ccc3c(c2)-c2cccc4cccc(c24)O3)c1 ; c1cc(-c2ccc3c(c2)-c2cccc4cccc(c24)O3)cc(N(c2ccc(-c3ccc(-n4c5ccccc5c5ccccc54)cc3)cc2)c2ccc(-c3ccc4ccccc4c3)cc2)c1]

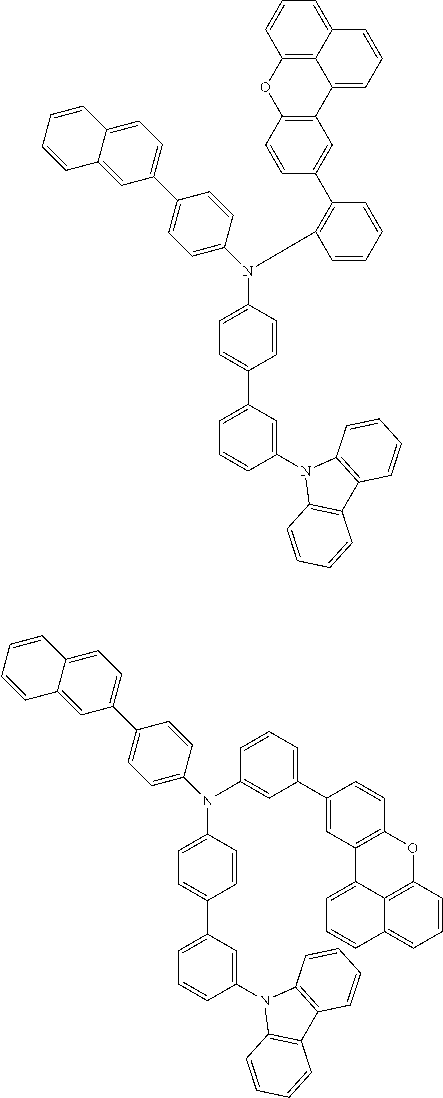 [SMILES: c1cc(-c2ccc(N(c3ccc(-c4ccc5ccccc5c4)cc3)c3ccccc3-c3ccc4c(c3)-c3cccc5cccc(c35)O4)cc2)cc(-n2c3ccccc3c3ccccc32)c1 ; c1cc(-c2ccc3c(c2)-c2cccc4cccc(c24)O3)cc(N(c2ccc(-c3cccc(-n4c5ccccc5c5ccccc54)c3)cc2)c2ccc(-c3ccc4ccccc4c3)cc2)c1]

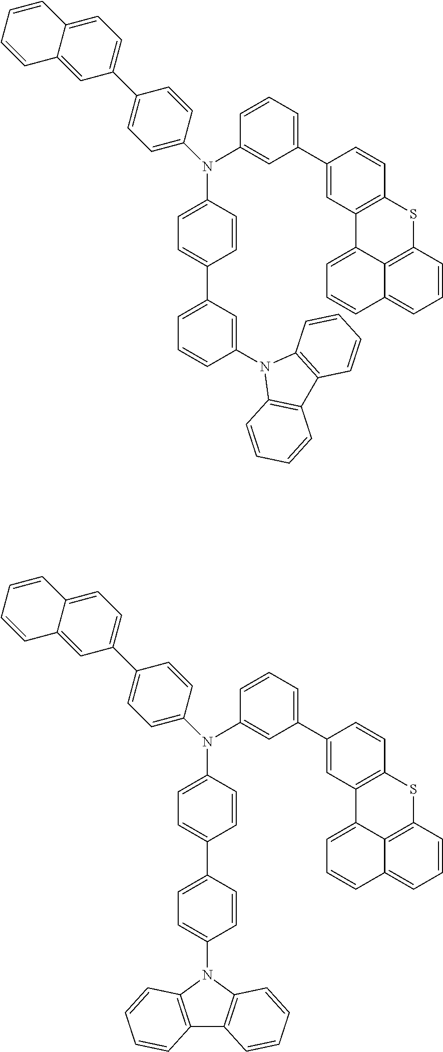 [SMILES: c1cc(-c2ccc3c(c2)-c2cccc4cccc(c24)S3)cc(N(c2ccc(-c3cccc(-n4c5ccccc5c5ccccc54)c3)cc2)c2ccc(-c3ccc4ccccc4c3)cc2)c1 ; c1cc(-c2ccc3c(c2)-c2cccc4cccc(c24)S3)cc(N(c2ccc(-c3ccc(-n4c5ccccc5c5ccccc54)cc3)cc2)c2ccc(-c3ccc4ccccc4c3)cc2)c1]

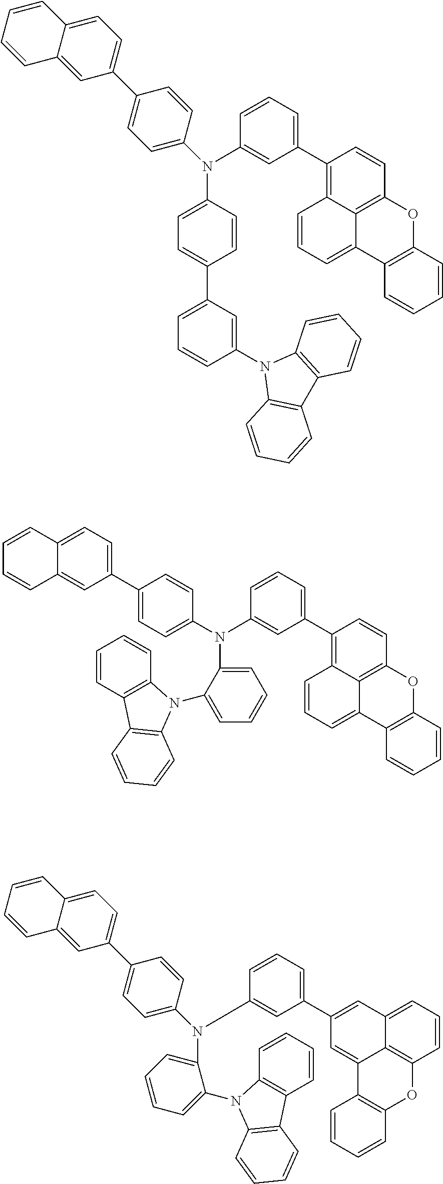 [SMILES: c1cc(-c2ccc3c4c(cccc24)-c2ccccc2O3)cc(N(c2ccc(-c3cccc(-n4c5ccccc5c5ccccc54)c3)cc2)c2ccc(-c3ccc4ccccc4c3)cc2)c1 ; c1cc(-c2ccc3c4c(cccc24)-c2ccccc2O3)cc(N(c2ccc(-c3ccc4ccccc4c3)cc2)c2ccccc2-n2c3ccccc3c3ccccc32)c1 ; c1cc(-c2cc3c4c(cccc4c2)Oc2ccccc2-3)cc(N(c2ccc(-c3ccc4ccccc4c3)cc2)c2ccccc2-n2c3ccccc3c3ccccc32)c1]

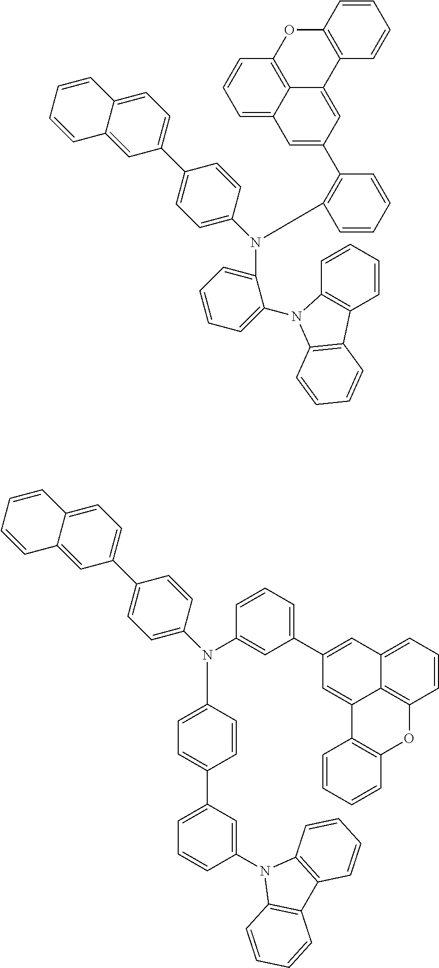 [SMILES: c1ccc2c(c1)Oc1cccc3cc(-c4ccccc4N(c4ccc(-c5ccc6ccccc6c5)cc4)c4ccccc4-n4c5ccccc5c5ccccc54)cc-2c13 ; c1cc(-c2cc3c4c(cccc4c2)Oc2ccccc2-3)cc(N(c2ccc(-c3cccc(-n4c5ccccc5c5ccccc54)c3)cc2)c2ccc(-c3ccc4ccccc4c3)cc2)c1]

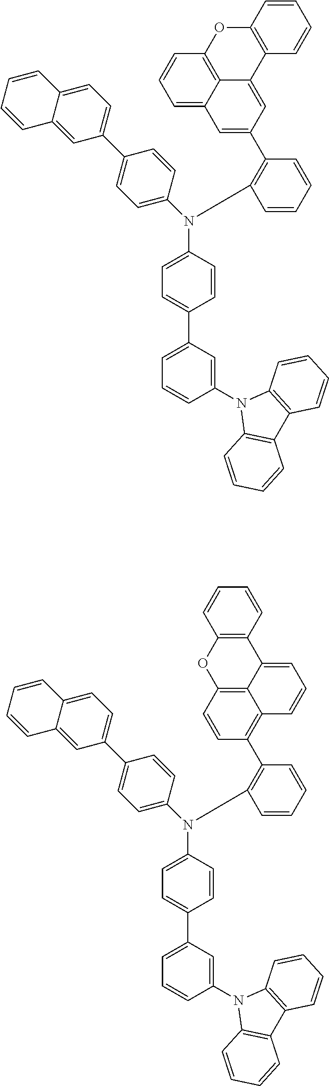 [SMILES: c1cc(-c2ccc(N(c3ccc(-c4ccc5ccccc5c4)cc3)c3ccccc3-c3cc4c5c(cccc5c3)Oc3ccccc3-4)cc2)cc(-n2c3ccccc3c3ccccc32)c1 ; c1cc(-c2ccc(N(c3ccc(-c4ccc5ccccc5c4)cc3)c3ccccc3-c3ccc4c5c(cccc35)-c3ccccc3O4)cc2)cc(-n2c3ccccc3c3ccccc32)c1]

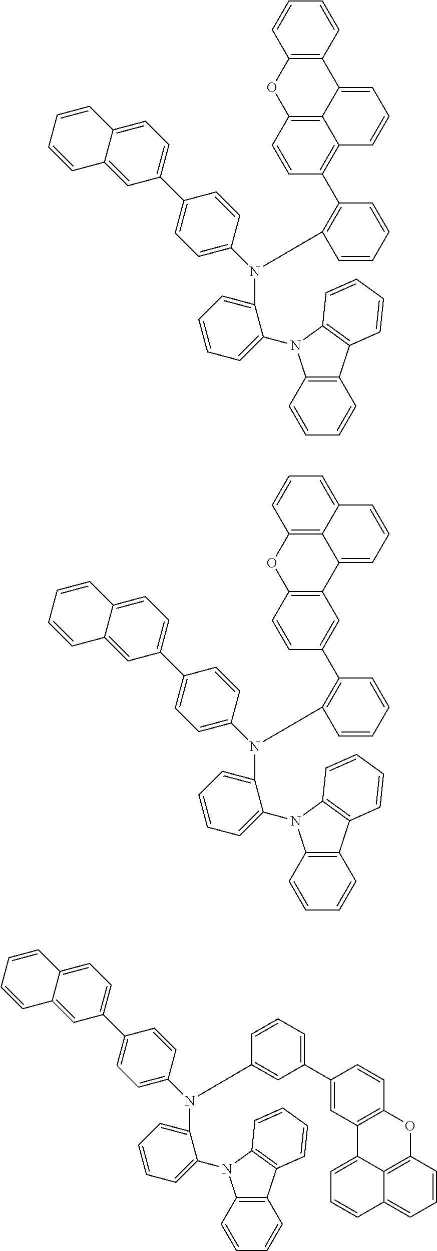

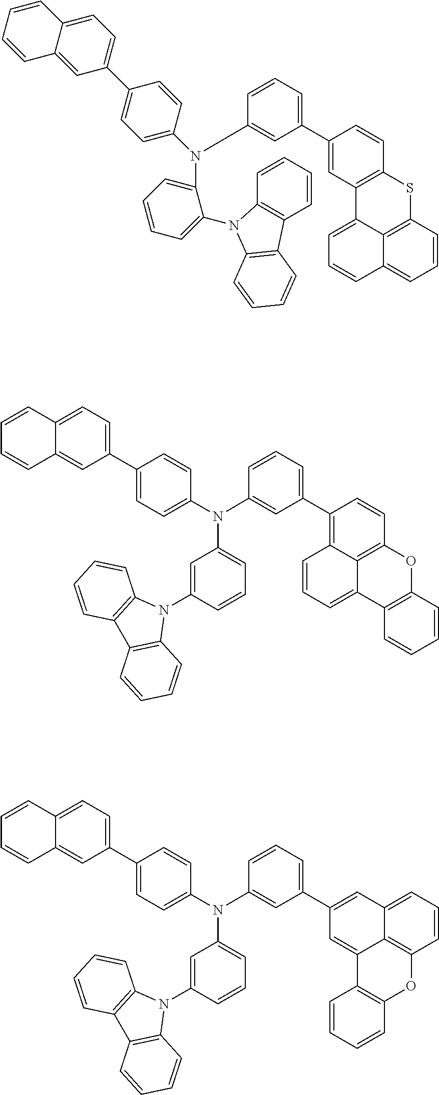 [SMILES: c1cc(-c2ccc3c(c2)-c2cccc4cccc(c24)S3)cc(N(c2ccc(-c3ccc4ccccc4c3)cc2)c2ccccc2-n2c3ccccc3c3ccccc32)c1 ; c1cc(-c2ccc3c4c(cccc24)-c2ccccc2O3)cc(N(c2ccc(-c3ccc4ccccc4c3)cc2)c2cccc(-n3c4ccccc4c4ccccc43)c2)c1 ; c1cc(-c2cc3c4c(cccc4c2)Oc2ccccc2-3)cc(N(c2ccc(-c3ccc4ccccc4c3)cc2)c2cccc(-n3c4ccccc4c4ccccc43)c2)c1]

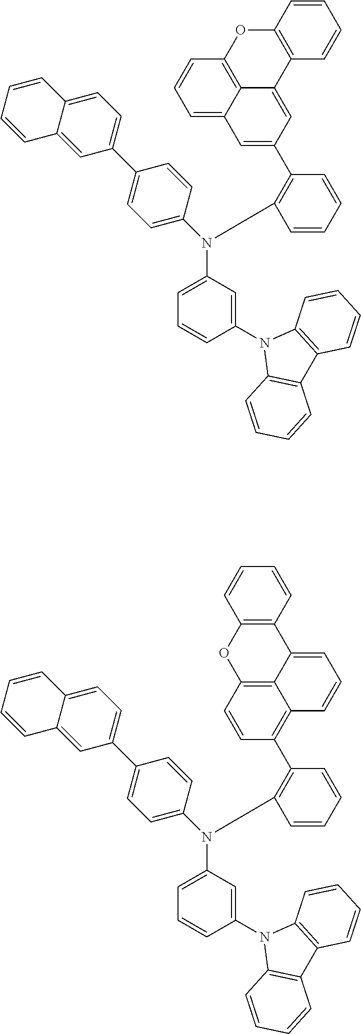 [SMILES: c1cc(N(c2ccc(-c3ccc4ccccc4c3)cc2)c2ccccc2-c2cc3c4c(cccc4c2)Oc2ccccc2-3)cc(-n2c3ccccc3c3ccccc32)c1 ; c1cc(N(c2ccc(-c3ccc4ccccc4c3)cc2)c2ccccc2-c2ccc3c4c(cccc24)-c2ccccc2O3)cc(-n2c3ccccc3c3ccccc32)c1]

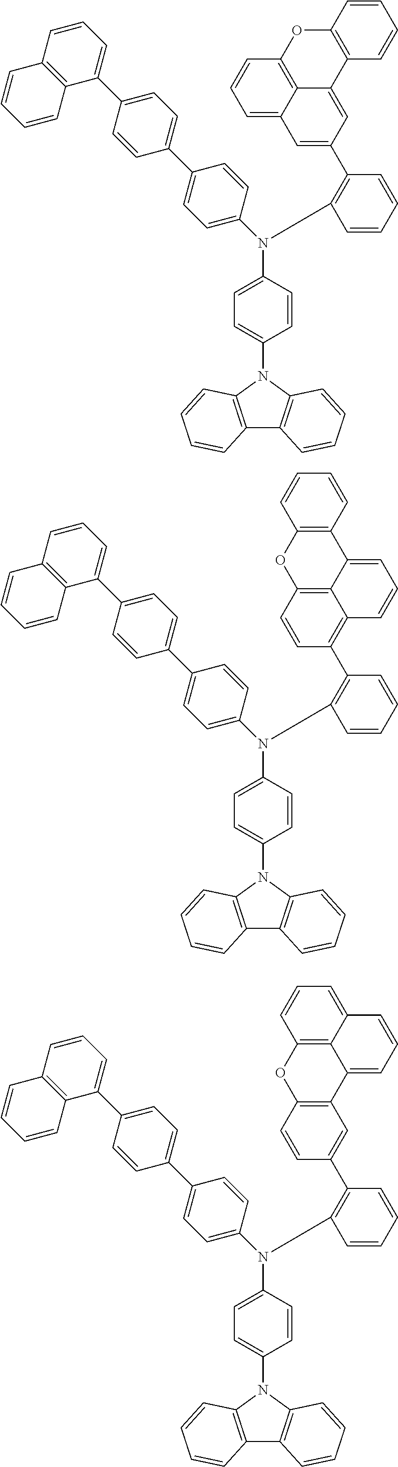

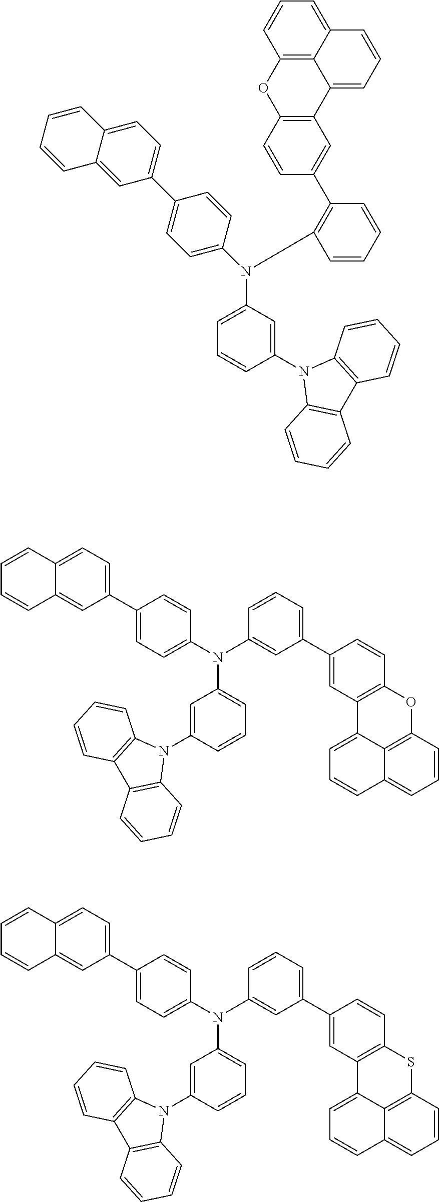 [SMILES: c1cc(N(c2ccc(-c3ccc4ccccc4c3)cc2)c2ccccc2-c2ccc3c(c2)-c2cccc4cccc(c24)O3)cc(-n2c3ccccc3c3ccccc32)c1 ; c1cc(-c2ccc3c(c2)-c2cccc4cccc(c24)O3)cc(N(c2ccc(-c3ccc4ccccc4c3)cc2)c2cccc(-n3c4ccccc4c4ccccc43)c2)c1 ; c1cc(-c2ccc3c(c2)-c2cccc4cccc(c24)S3)cc(N(c2ccc(-c3ccc4ccccc4c3)cc2)c2cccc(-n3c4ccccc4c4ccccc43)c2)c1]

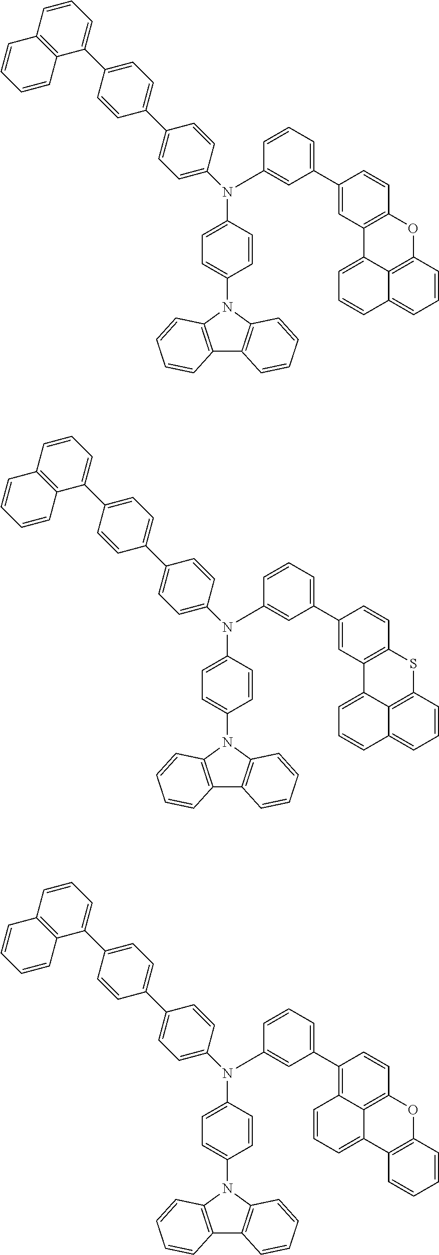 [SMILES: c1cc(-c2ccc3c(c2)-c2cccc4cccc(c24)O3)cc(N(c2ccc(-c3ccc(-c4cccc5ccccc45)cc3)cc2)c2ccc(-n3c4ccccc4c4ccccc43)cc2)c1 ; c1cc(-c2ccc3c(c2)-c2cccc4cccc(c24)S3)cc(N(c2ccc(-c3ccc(-c4cccc5ccccc45)cc3)cc2)c2ccc(-n3c4ccccc4c4ccccc43)cc2)c1 ; c1cc(-c2ccc3c4c(cccc24)-c2ccccc2O3)cc(N(c2ccc(-c3ccc(-c4cccc5ccccc45)cc3)cc2)c2ccc(-n3c4ccccc4c4ccccc43)cc2)c1]

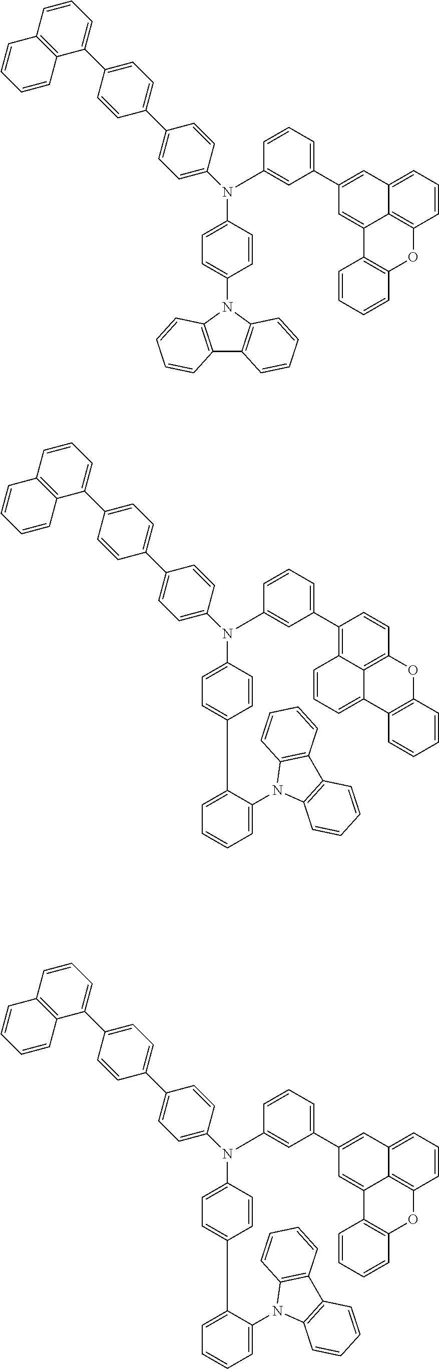 [SMILES: c1cc(-c2cc3c4c(cccc4c2)Oc2ccccc2-3)cc(N(c2ccc(-c3ccc(-c4cccc5ccccc45)cc3)cc2)c2ccc(-n3c4ccccc4c4ccccc43)cc2)c1 ; c1cc(-c2ccc3c4c(cccc24)-c2ccccc2O3)cc(N(c2ccc(-c3ccc(-c4cccc5ccccc45)cc3)cc2)c2ccc(-c3ccccc3-n3c4ccccc4c4ccccc43)cc2)c1 ; c1cc(-c2cc3c4c(cccc4c2)Oc2ccccc2-3)cc(N(c2ccc(-c3ccc(-c4cccc5ccccc45)cc3)cc2)c2ccc(-c3ccccc3-n3c4ccccc4c4ccccc43)cc2)c1]

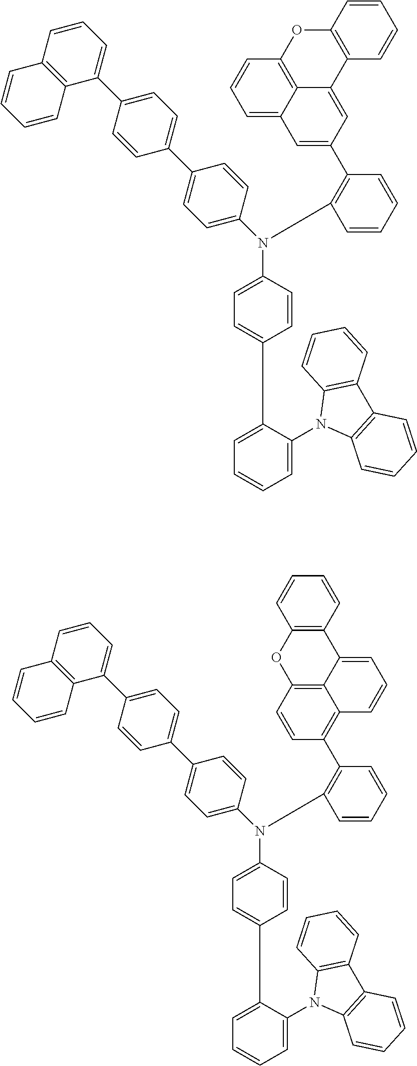 [SMILES: c1ccc2c(c1)Oc1cccc3cc(-c4ccccc4N(c4ccc(-c5ccc(-c6cccc7ccccc67)cc5)cc4)c4ccc(-c5ccccc5-n5c6ccccc6c6ccccc65)cc4)cc-2c13 ; c1ccc2c(c1)Oc1ccc(-c3ccccc3N(c3ccc(-c4ccc(-c5cccc6ccccc56)cc4)cc3)c3ccc(-c4ccccc4-n4c5ccccc5c5ccccc54)cc3)c3cccc-2c13]

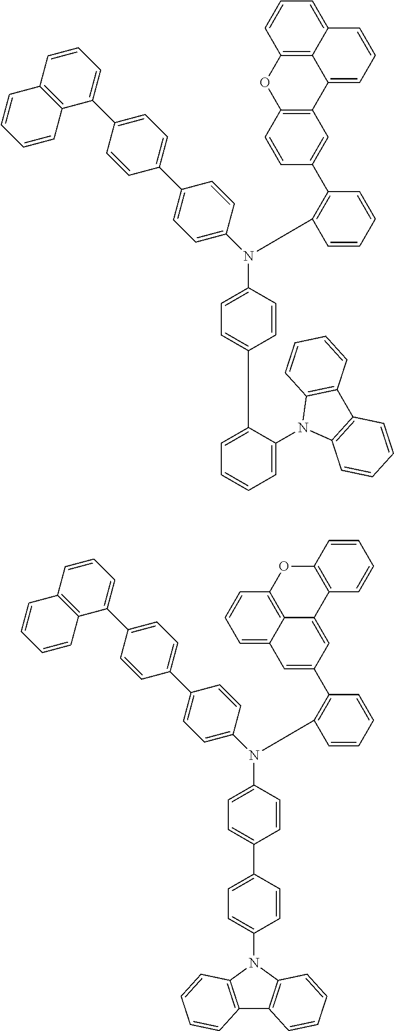 [SMILES: c1ccc(N(c2ccc(-c3ccc(-c4cccc5ccccc45)cc3)cc2)c2ccc(-c3ccccc3-n3c4ccccc4c4ccccc43)cc2)c(-c2ccc3c(c2)-c2cccc4cccc(c24)O3)c1 ; c1ccc2c(c1)Oc1cccc3cc(-c4ccccc4N(c4ccc(-c5ccc(-c6cccc7ccccc67)cc5)cc4)c4ccc(-c5ccc(-n6c7ccccc7c7ccccc76)cc5)cc4)cc-2c13]

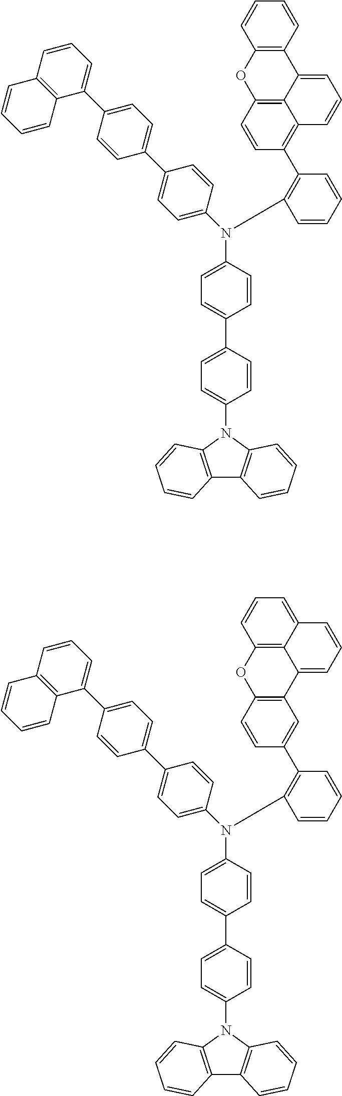 [SMILES: c1ccc2c(c1)Oc1ccc(-c3ccccc3N(c3ccc(-c4ccc(-c5cccc6ccccc56)cc4)cc3)c3ccc(-c4ccc(-n5c6ccccc6c6ccccc65)cc4)cc3)c3cccc-2c13 ; c1ccc(N(c2ccc(-c3ccc(-c4cccc5ccccc45)cc3)cc2)c2ccc(-c3ccc(-n4c5ccccc5c5ccccc54)cc3)cc2)c(-c2ccc3c(c2)-c2cccc4cccc(c24)O3)c1]

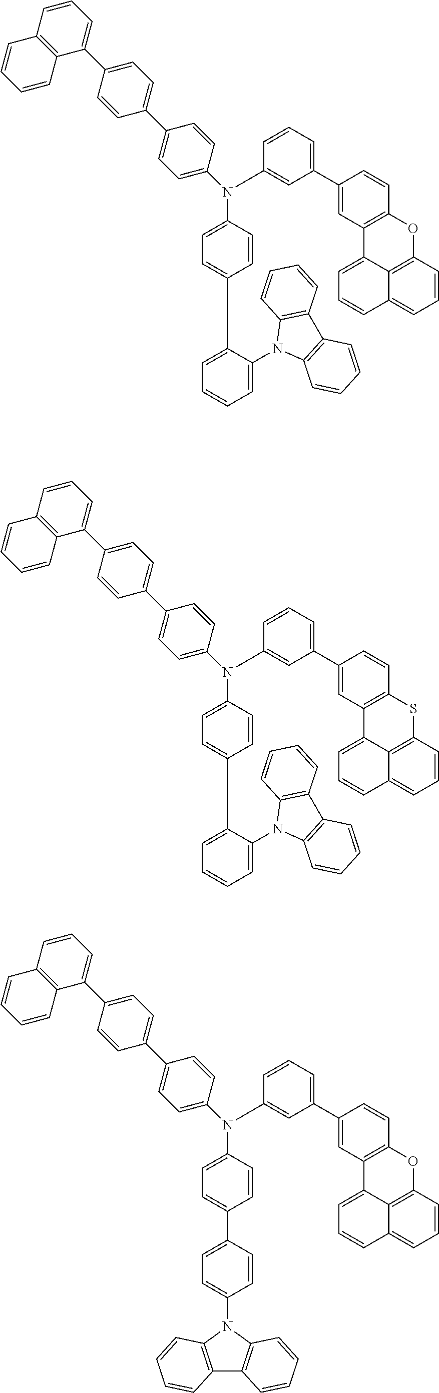 [SMILES: c1cc(-c2ccc3c(c2)-c2cccc4cccc(c24)O3)cc(N(c2ccc(-c3ccc(-c4cccc5ccccc45)cc3)cc2)c2ccc(-c3ccccc3-n3c4ccccc4c4ccccc43)cc2)c1 ; c1cc(-c2ccc3c(c2)-c2cccc4cccc(c24)S3)cc(N(c2ccc(-c3ccc(-c4cccc5ccccc45)cc3)cc2)c2ccc(-c3ccccc3-n3c4ccccc4c4ccccc43)cc2)c1 ; c1cc(-c2ccc3c(c2)-c2cccc4cccc(c24)O3)cc(N(c2ccc(-c3ccc(-c4cccc5ccccc45)cc3)cc2)c2ccc(-c3ccc(-n4c5ccccc5c5ccccc54)cc3)cc2)c1]

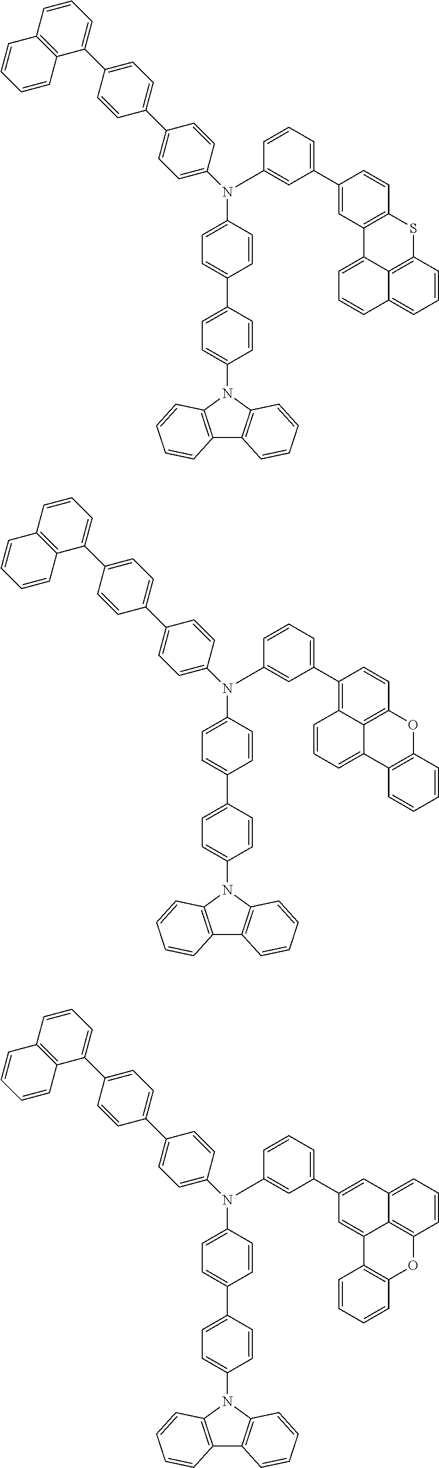 [SMILES: c1cc(-c2ccc3c(c2)-c2cccc4cccc(c24)S3)cc(N(c2ccc(-c3ccc(-c4cccc5ccccc45)cc3)cc2)c2ccc(-c3ccc(-n4c5ccccc5c5ccccc54)cc3)cc2)c1 ; c1cc(-c2ccc3c4c(cccc24)-c2ccccc2O3)cc(N(c2ccc(-c3ccc(-c4cccc5ccccc45)cc3)cc2)c2ccc(-c3ccc(-n4c5ccccc5c5ccccc54)cc3)cc2)c1 ; c1cc(-c2cc3c4c(cccc4c2)Oc2ccccc2-3)cc(N(c2ccc(-c3ccc(-c4cccc5ccccc45)cc3)cc2)c2ccc(-c3ccc(-n4c5ccccc5c5ccccc54)cc3)cc2)c1]

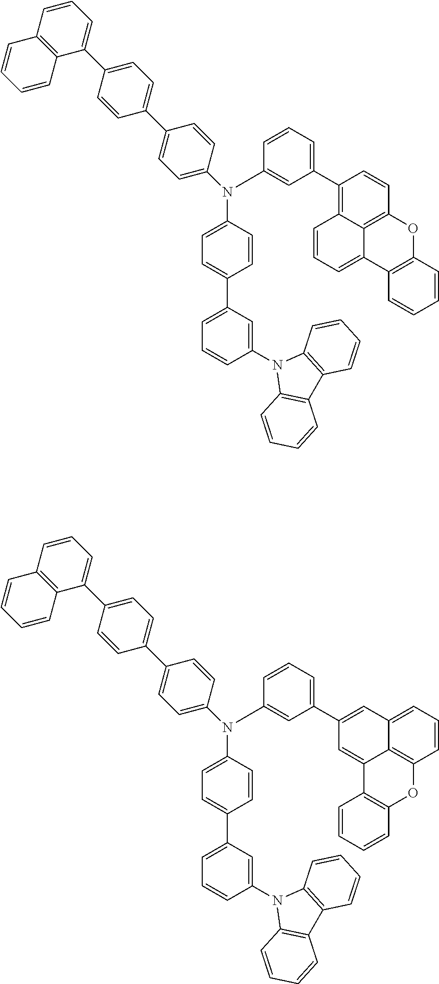 [SMILES: c1cc(-c2ccc3c4c(cccc24)-c2ccccc2O3)cc(N(c2ccc(-c3ccc(-c4cccc5ccccc45)cc3)cc2)c2ccc(-c3cccc(-n4c5ccccc5c5ccccc54)c3)cc2)c1 ; c1cc(-c2cc3c4c(cccc4c2)Oc2ccccc2-3)cc(N(c2ccc(-c3ccc(-c4cccc5ccccc45)cc3)cc2)c2ccc(-c3cccc(-n4c5ccccc5c5ccccc54)c3)cc2)c1]

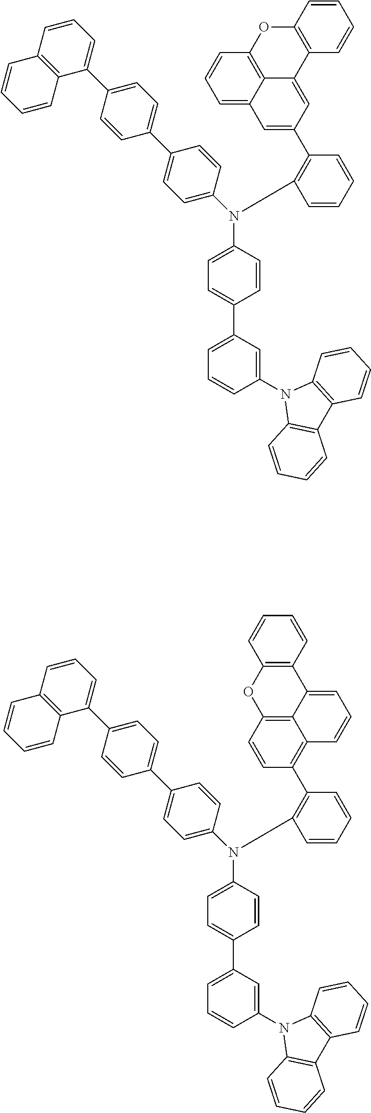 [SMILES: c1cc(-c2ccc(N(c3ccc(-c4ccc(-c5cccc6ccccc56)cc4)cc3)c3ccccc3-c3cc4c5c(cccc5c3)Oc3ccccc3-4)cc2)cc(-n2c3ccccc3c3ccccc32)c1 ; c1cc(-c2ccc(N(c3ccc(-c4ccc(-c5cccc6ccccc56)cc4)cc3)c3ccccc3-c3ccc4c5c(cccc35)-c3ccccc3O4)cc2)cc(-n2c3ccccc3c3ccccc32)c1]

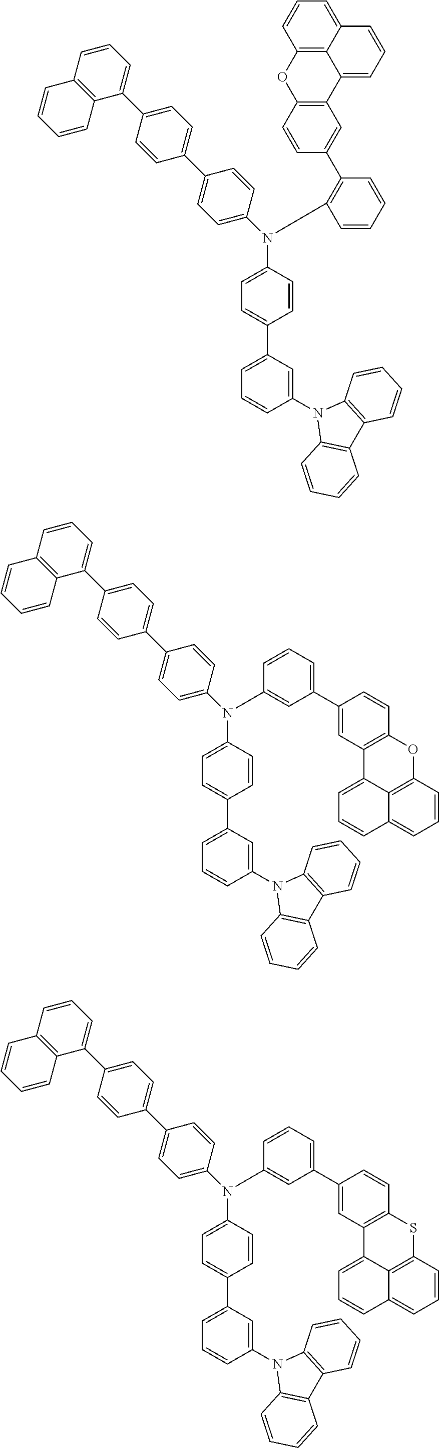 [SMILES: c1cc(-c2ccc(N(c3ccc(-c4ccc(-c5cccc6ccccc56)cc4)cc3)c3ccccc3-c3ccc4c(c3)-c3cccc5cccc(c35)O4)cc2)cc(-n2c3ccccc3c3ccccc32)c1 ; c1cc(-c2ccc3c(c2)-c2cccc4cccc(c24)O3)cc(N(c2ccc(-c3ccc(-c4cccc5ccccc45)cc3)cc2)c2ccc(-c3cccc(-n4c5ccccc5c5ccccc54)c3)cc2)c1 ; c1cc(-c2ccc3c(c2)-c2cccc4cccc(c24)S3)cc(N(c2ccc(-c3ccc(-c4cccc5ccccc45)cc3)cc2)c2ccc(-c3cccc(-n4c5ccccc5c5ccccc54)c3)cc2)c1]

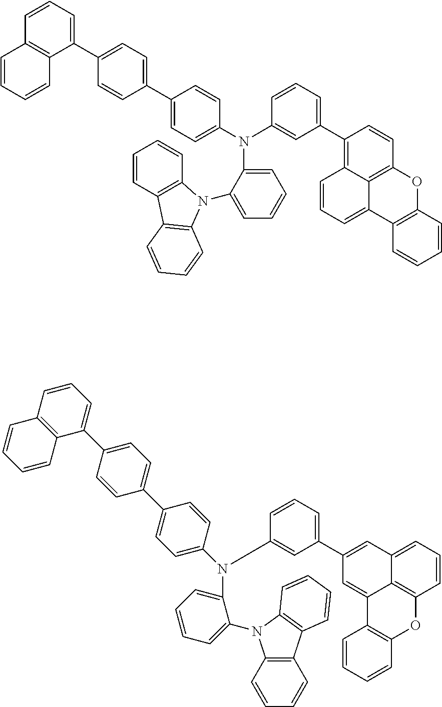 [SMILES: c1cc(-c2ccc3c4c(cccc24)-c2ccccc2O3)cc(N(c2ccc(-c3ccc(-c4cccc5ccccc45)cc3)cc2)c2ccccc2-n2c3ccccc3c3ccccc32)c1 ; c1cc(-c2cc3c4c(cccc4c2)Oc2ccccc2-3)cc(N(c2ccc(-c3ccc(-c4cccc5ccccc45)cc3)cc2)c2ccccc2-n2c3ccccc3c3ccccc32)c1]

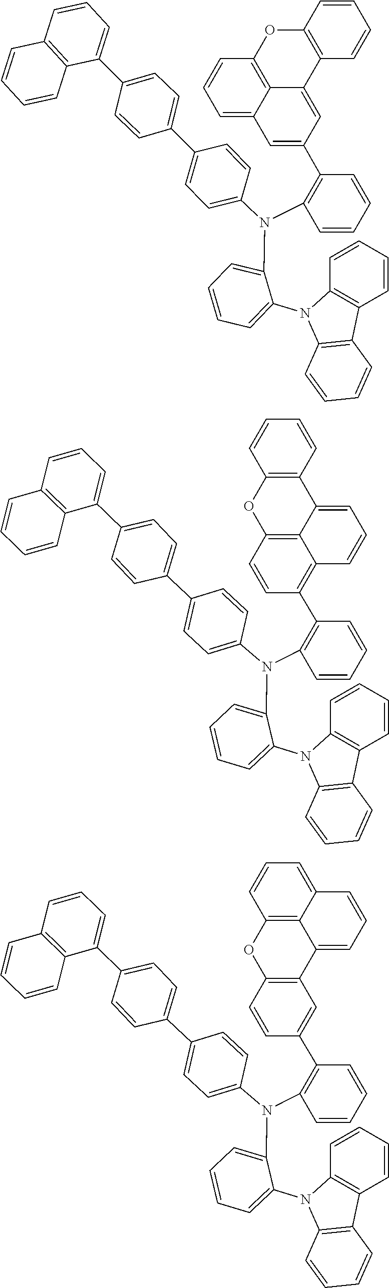

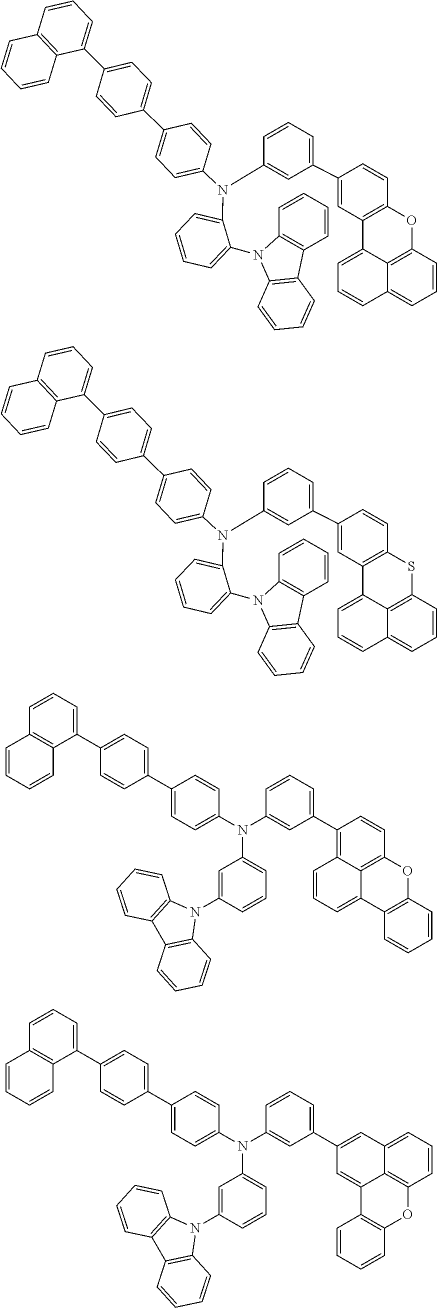 [SMILES: c1cc(-c2ccc3c(c2)-c2cccc4cccc(c24)O3)cc(N(c2ccc(-c3ccc(-c4cccc5ccccc45)cc3)cc2)c2ccccc2-n2c3ccccc3c3ccccc32)c1 ; c1cc(-c2ccc3c(c2)-c2cccc4cccc(c24)S3)cc(N(c2ccc(-c3ccc(-c4cccc5ccccc45)cc3)cc2)c2ccccc2-n2c3ccccc3c3ccccc32)c1 ; c1cc(-c2ccc3c4c(cccc24)-c2ccccc2O3)cc(N(c2ccc(-c3ccc(-c4cccc5ccccc45)cc3)cc2)c2cccc(-n3c4ccccc4c4ccccc43)c2)c1 ; c1cc(-c2cc3c4c(cccc4c2)Oc2ccccc2-3)cc(N(c2ccc(-c3ccc(-c4cccc5ccccc45)cc3)cc2)c2cccc(-n3c4ccccc4c4ccccc43)c2)c1]

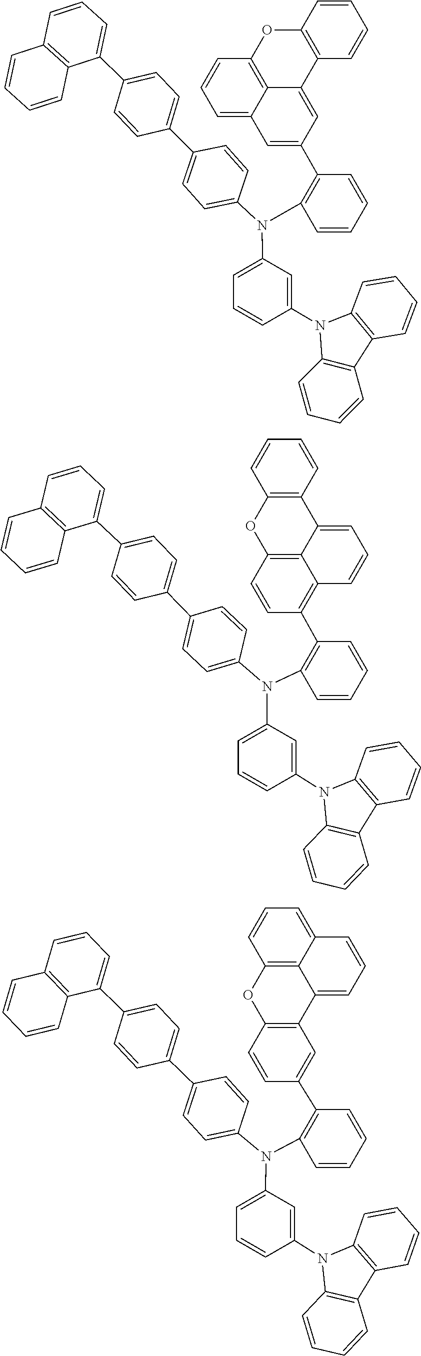

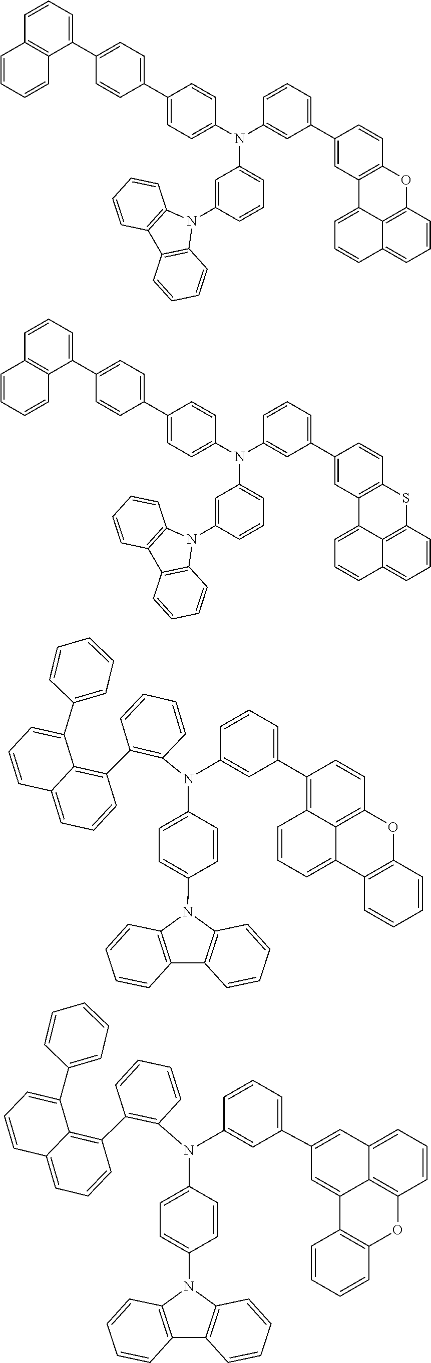

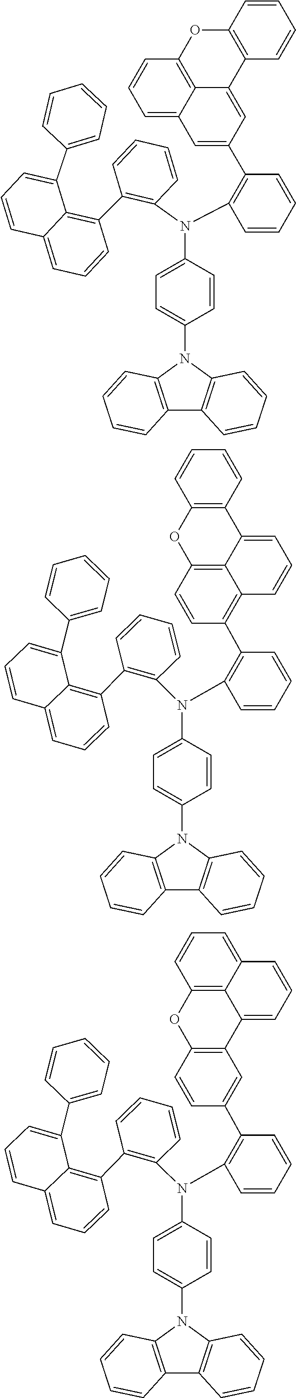

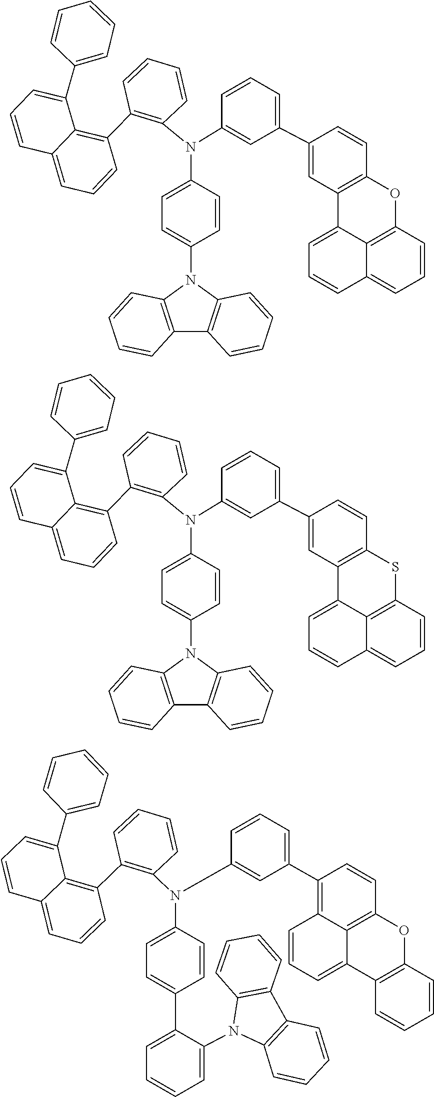

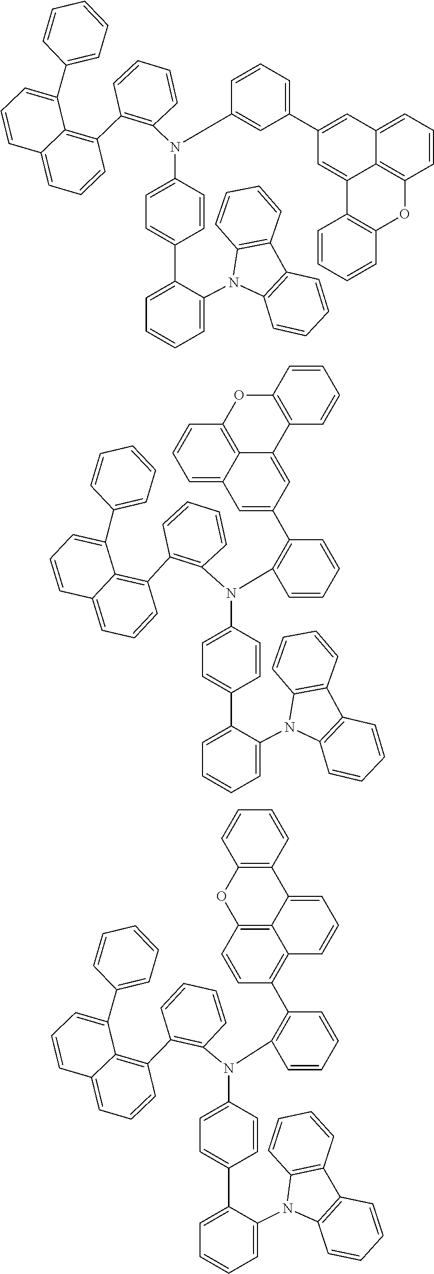

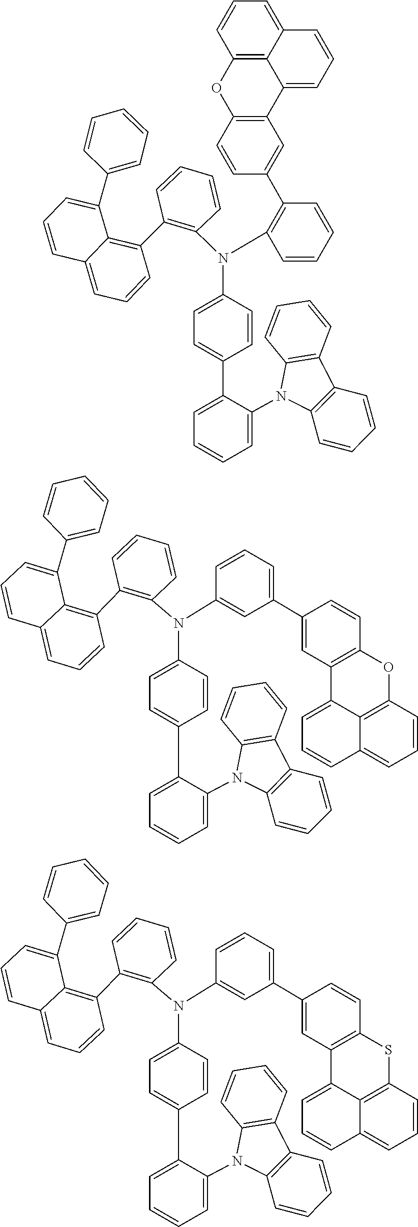 [SMILES: c1ccc(-c2cccc3cccc(-c4ccccc4N(c4ccc(-c5ccccc5-n5c6ccccc6c6ccccc65)cc4)c4ccccc4-c4ccc5c(c4)-c4cccc6cccc(c46)O5)c23)cc1 ; c1ccc(-c2cccc3cccc(-c4ccccc4N(c4ccc(-c5ccccc5-n5c6ccccc6c6ccccc65)cc4)c4cccc(-c5ccc6c(c5)-c5cccc7cccc(c57)O6)c4)c23)cc1 ; c1ccc(-c2cccc3cccc(-c4ccccc4N(c4ccc(-c5ccccc5-n5c6ccccc6c6ccccc65)cc4)c4cccc(-c5ccc6c(c5)-c5cccc7cccc(c57)S6)c4)c23)cc1]

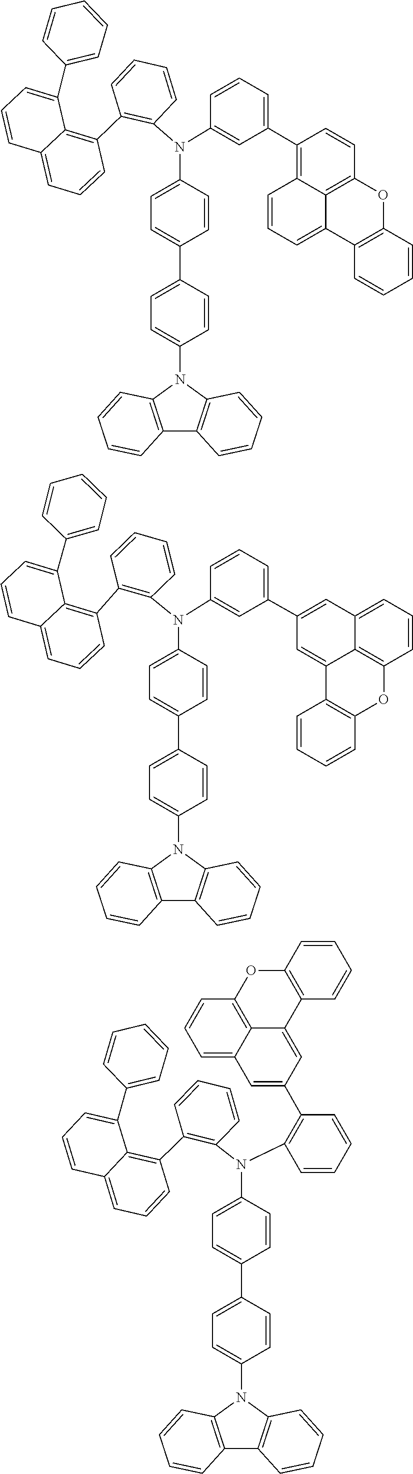

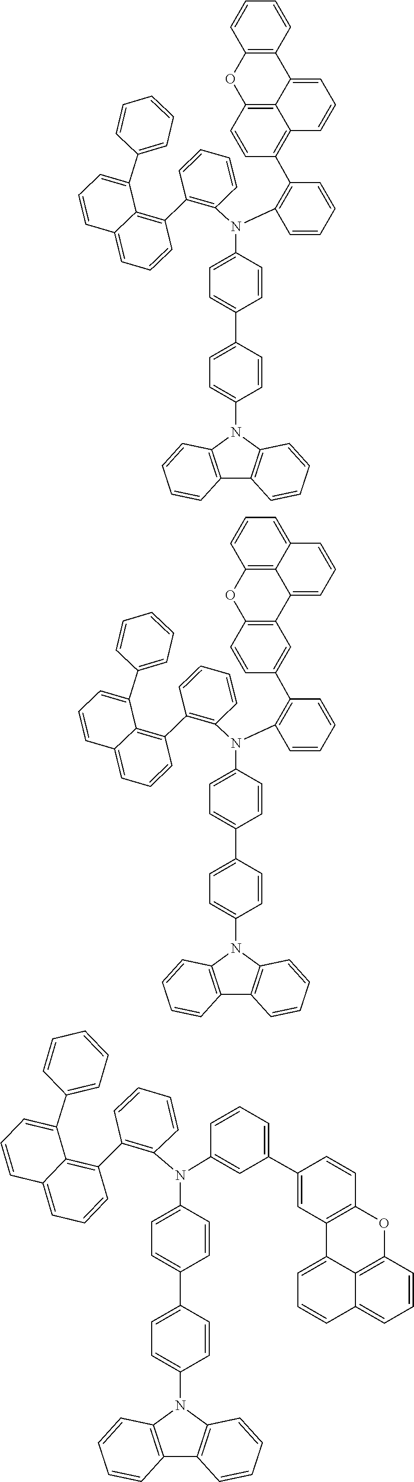

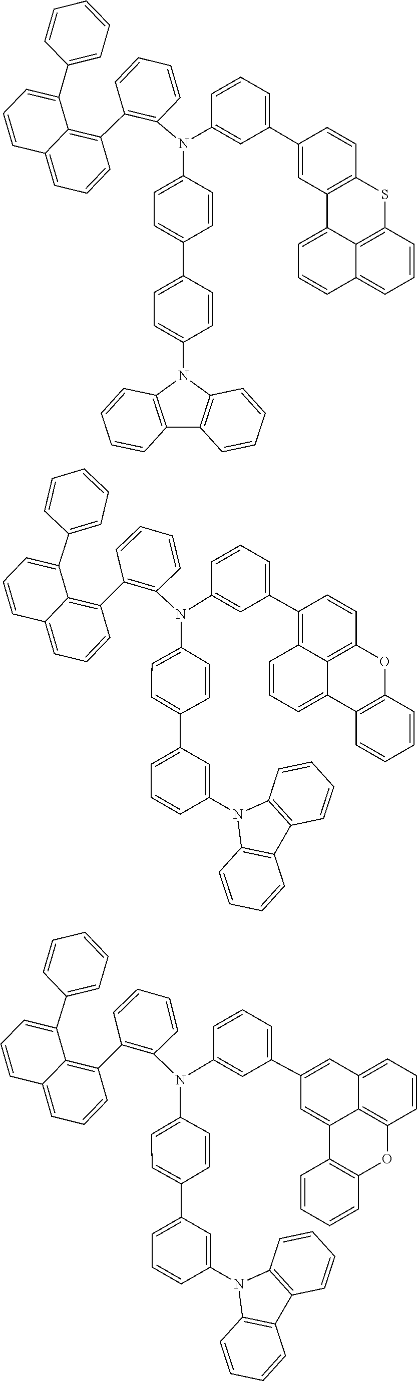

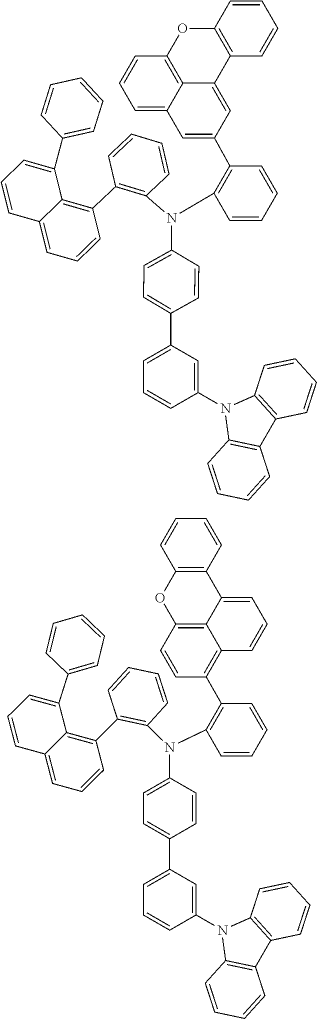

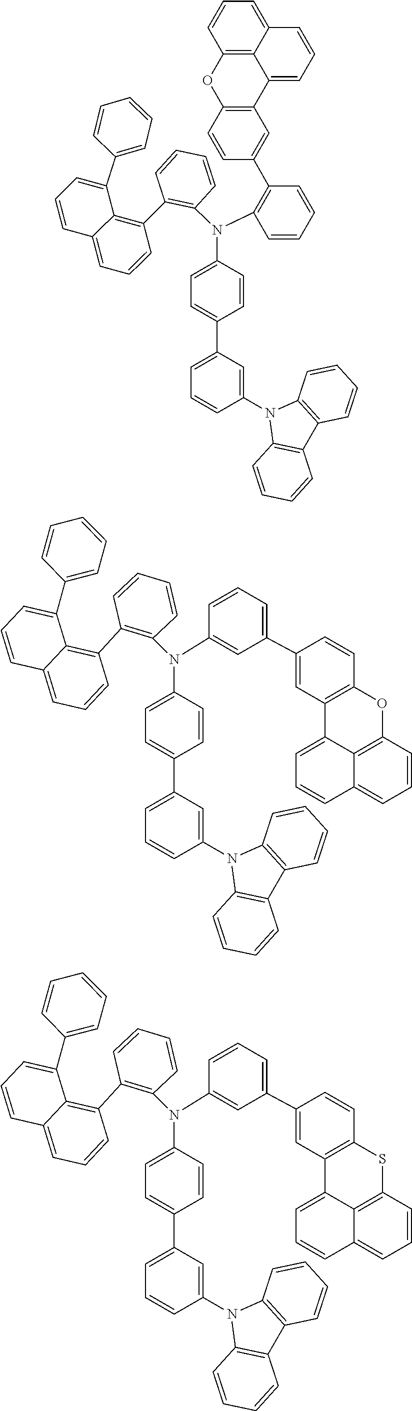 [SMILES: c1ccc(-c2cccc3cccc(-c4ccccc4N(c4ccc(-c5cccc(-n6c7ccccc7c7ccccc76)c5)cc4)c4ccccc4-c4ccc5c(c4)-c4cccc6cccc(c46)O5)c23)cc1 ; c1ccc(-c2cccc3cccc(-c4ccccc4N(c4ccc(-c5cccc(-n6c7ccccc7c7ccccc76)c5)cc4)c4cccc(-c5ccc6c(c5)-c5cccc7cccc(c57)O6)c4)c23)cc1 ; c1ccc(-c2cccc3cccc(-c4ccccc4N(c4ccc(-c5cccc(-n6c7ccccc7c7ccccc76)c5)cc4)c4cccc(-c5ccc6c(c5)-c5cccc7cccc(c57)S6)c4)c23)cc1]

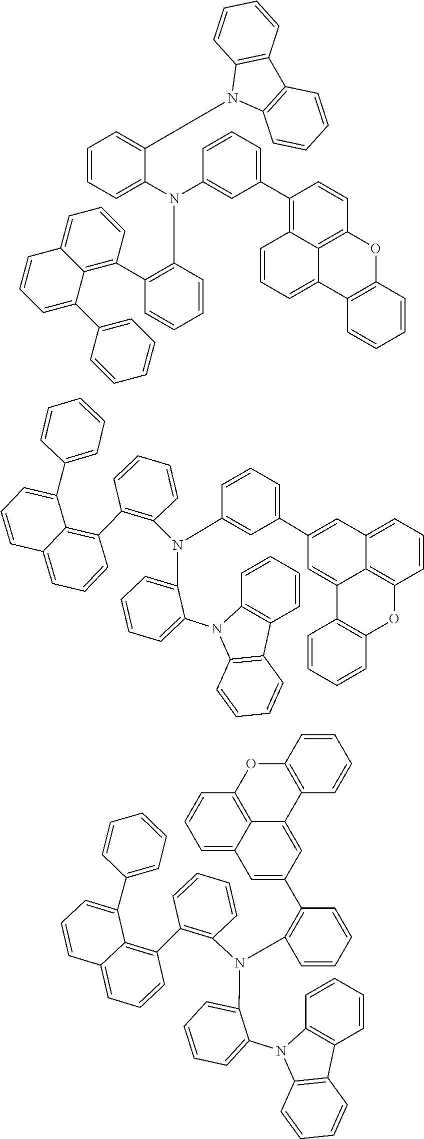

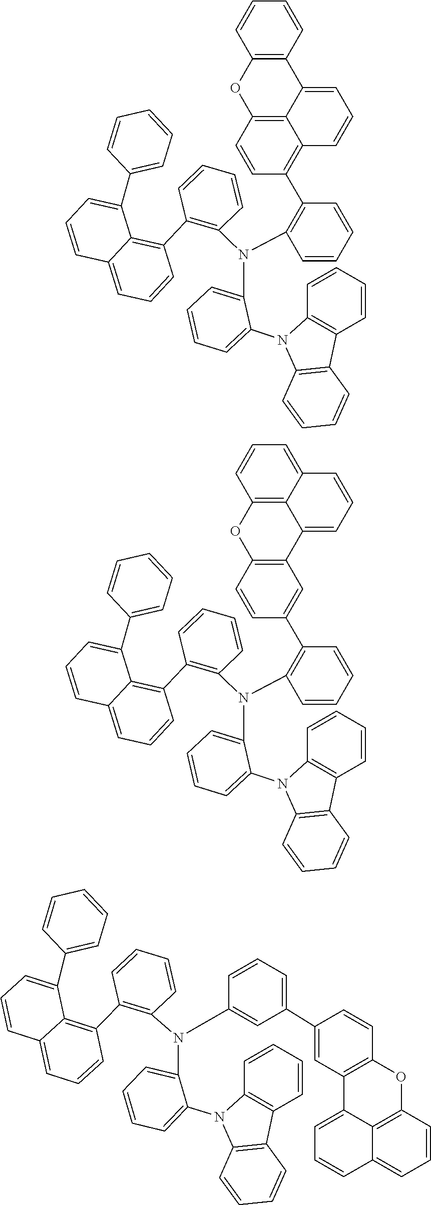

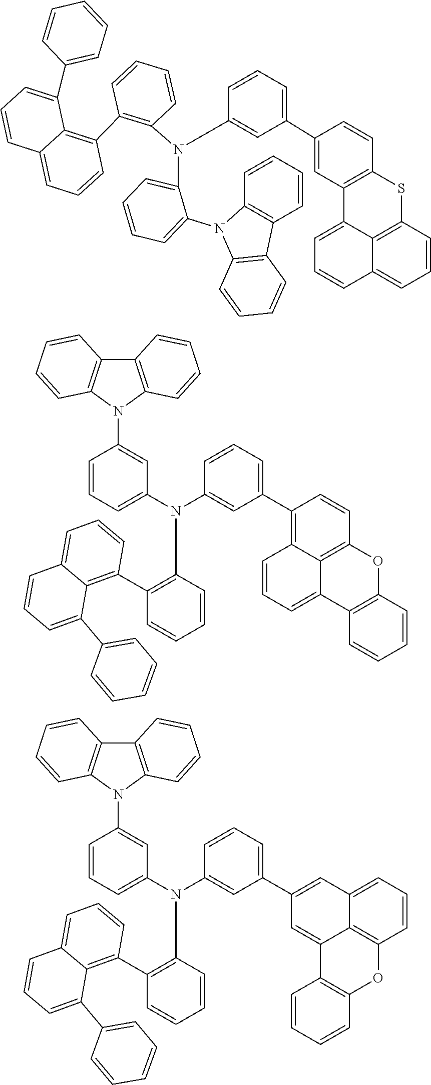

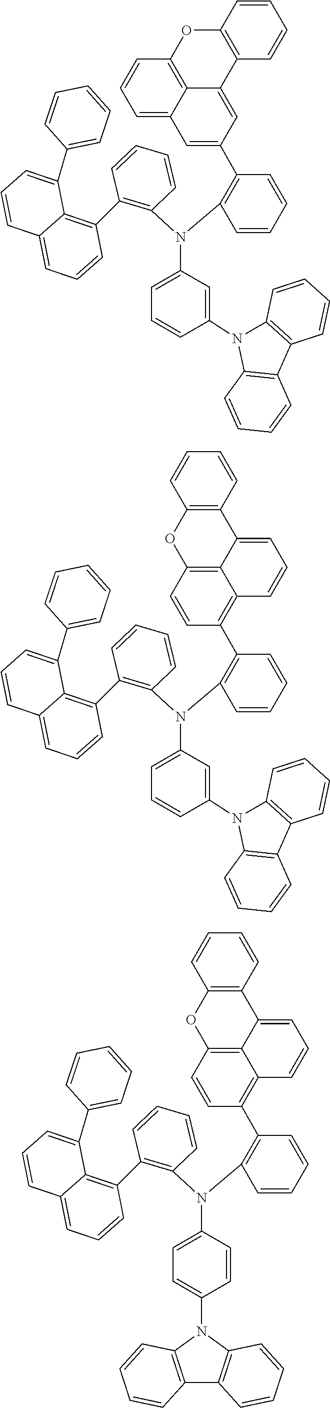

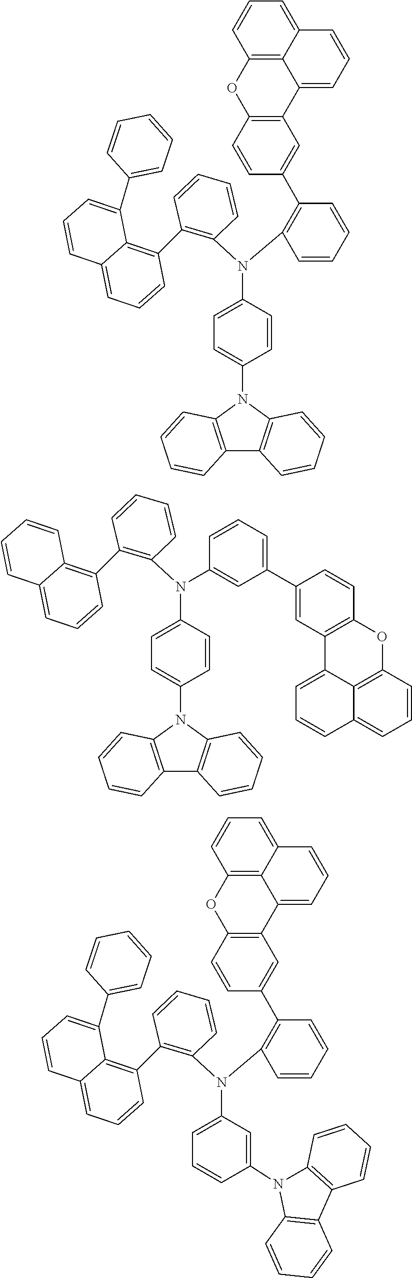

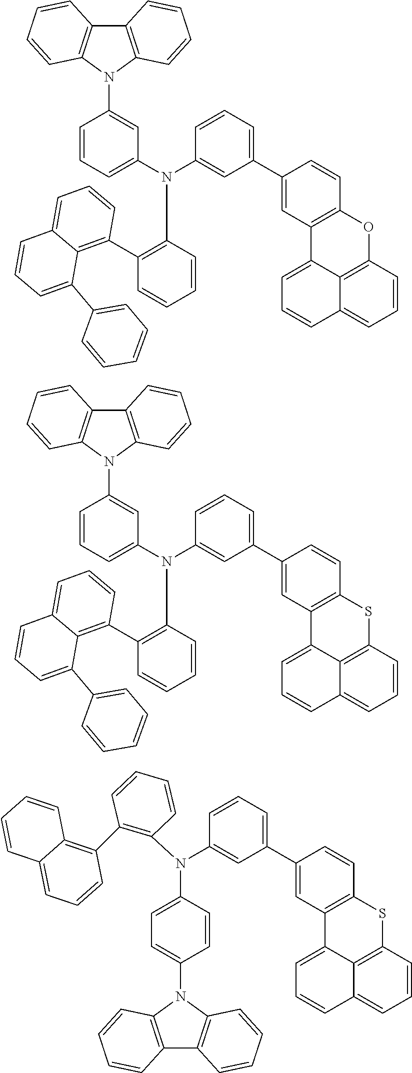

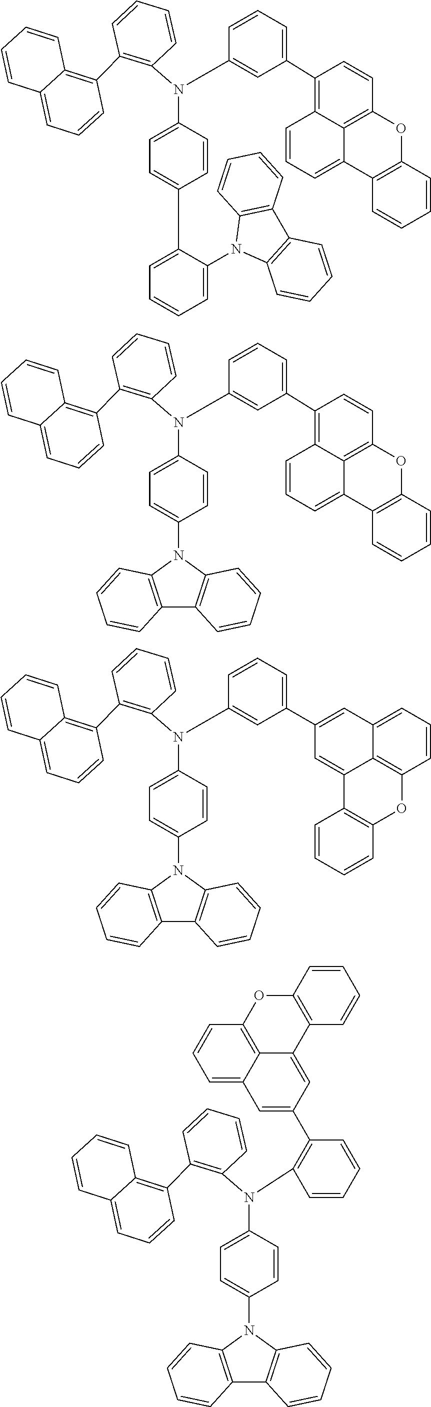

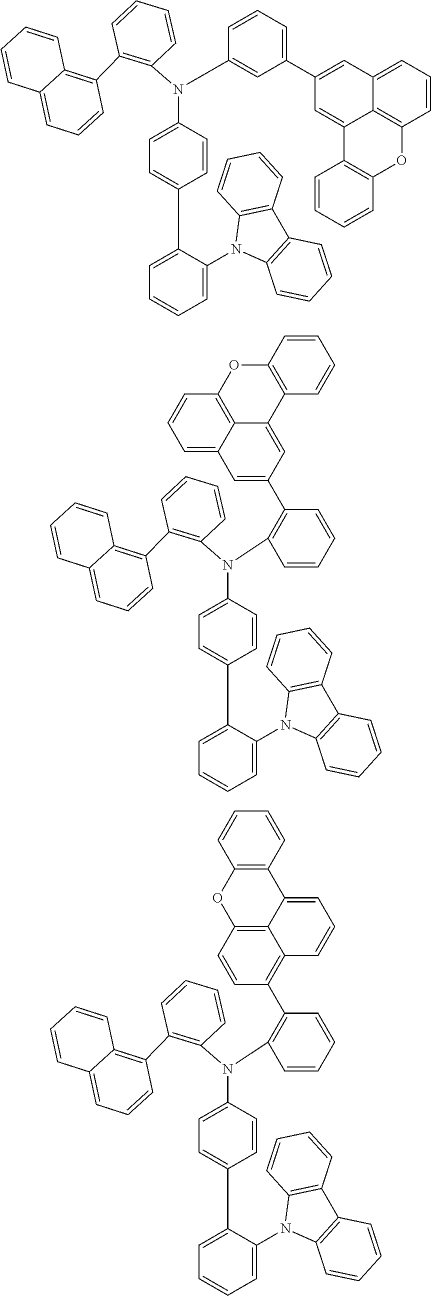

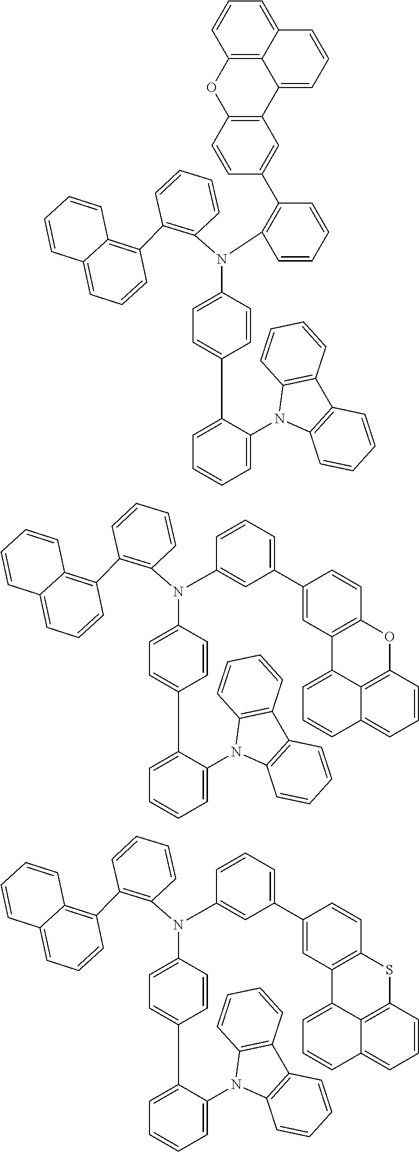

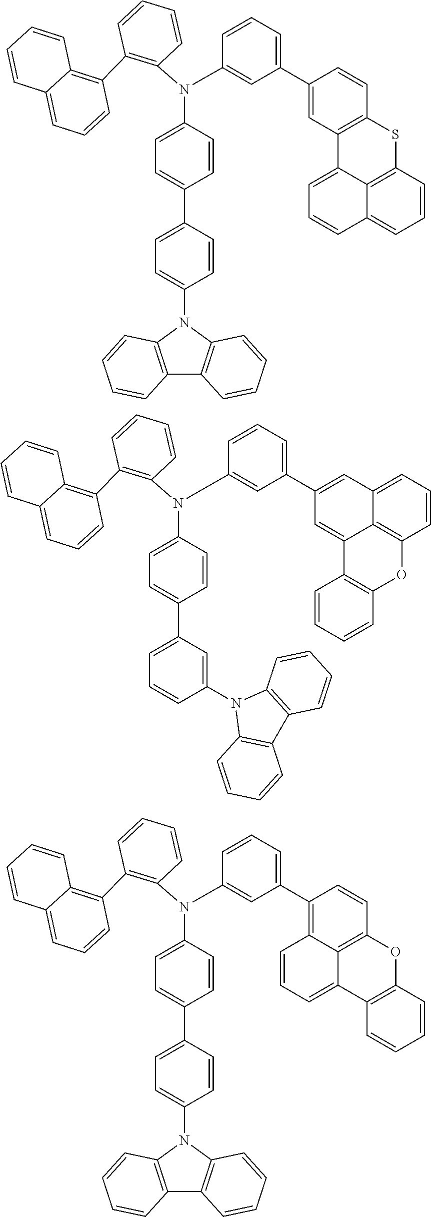

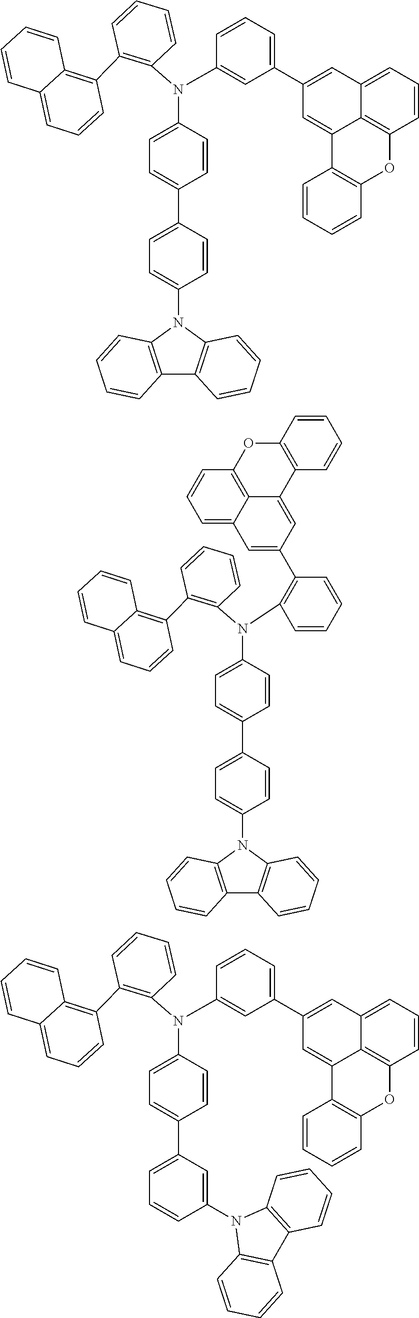

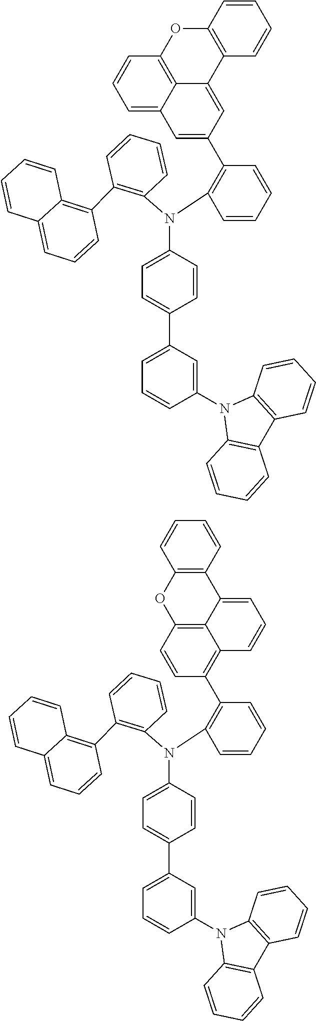

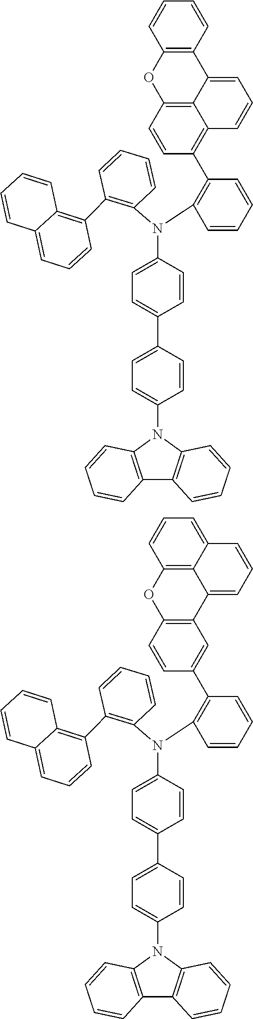

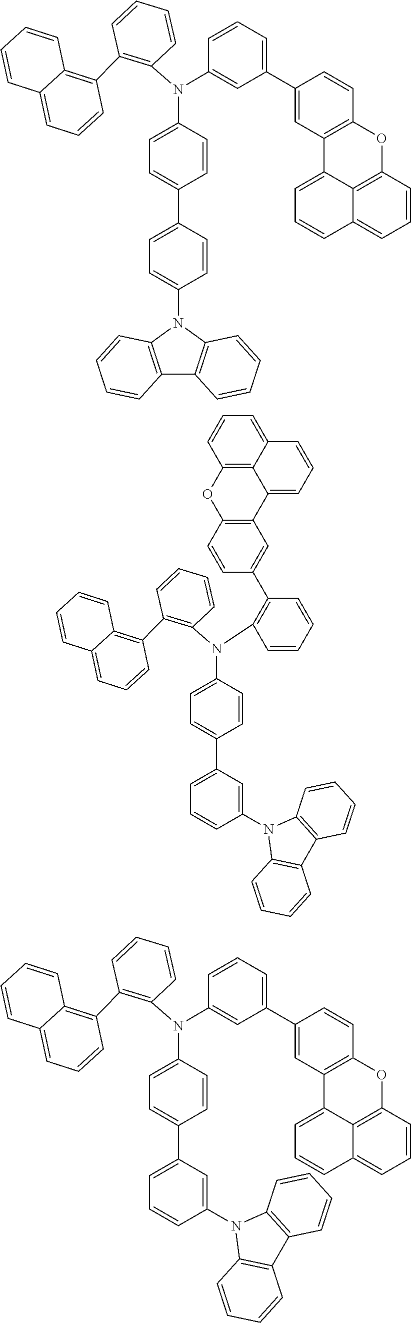

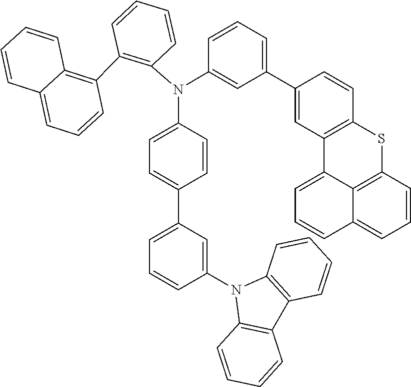 [SMILES: c1cc(-c2ccc3c(c2)-c2cccc4cccc(c24)S3)cc(N(c2ccc(-c3cccc(-n4c5ccccc5c5ccccc54)c3)cc2)c2ccccc2-c2cccc3ccccc23)c1]

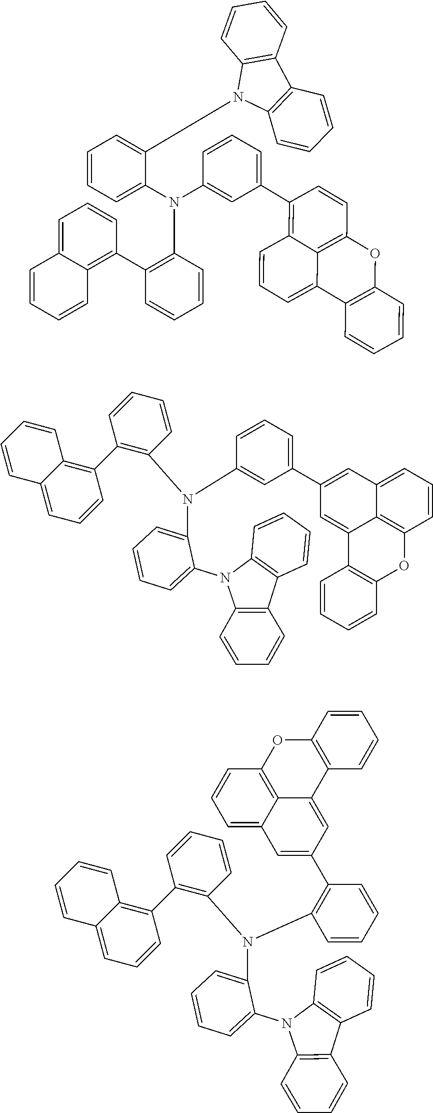 [SMILES: c1cc(-c2ccc3c4c(cccc24)-c2ccccc2O3)cc(N(c2ccccc2-c2cccc3ccccc23)c2ccccc2-n2c3ccccc3c3ccccc32)c1 ; c1cc(-c2cc3c4c(cccc4c2)Oc2ccccc2-3)cc(N(c2ccccc2-c2cccc3ccccc23)c2ccccc2-n2c3ccccc3c3ccccc32)c1 ; c1ccc2c(c1)Oc1cccc3cc(-c4ccccc4N(c4ccccc4-c4cccc5ccccc45)c4ccccc4-n4c5ccccc5c5ccccc54)cc-2c13]

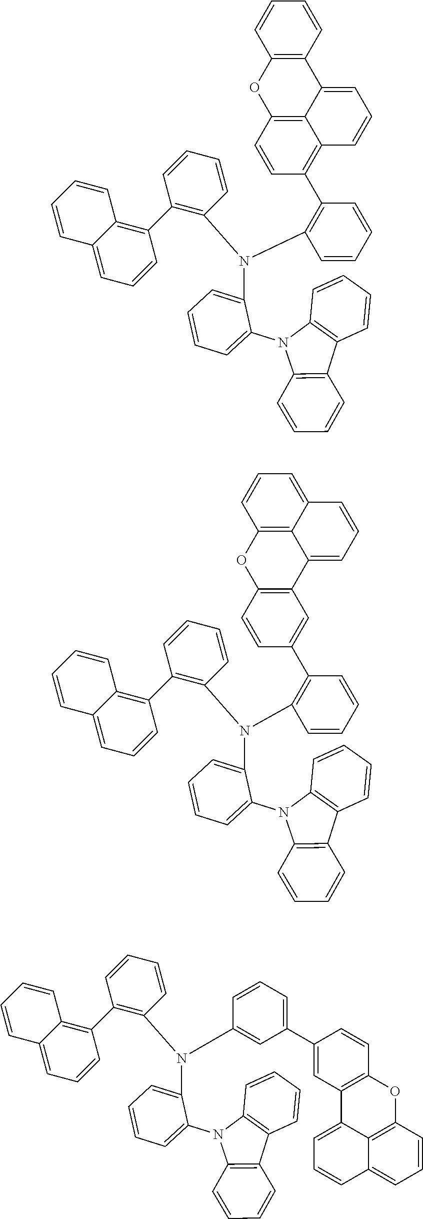

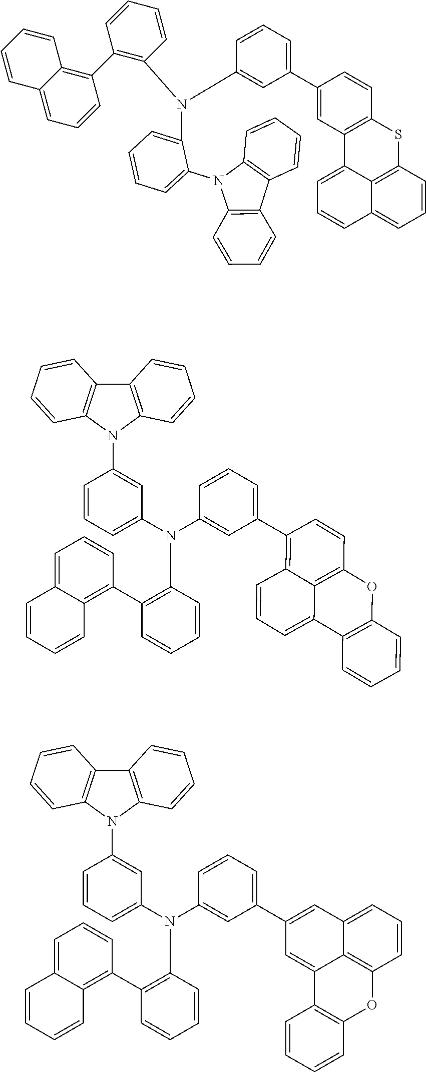 [SMILES: c1cc(-c2ccc3c(c2)-c2cccc4cccc(c24)S3)cc(N(c2ccccc2-c2cccc3ccccc23)c2ccccc2-n2c3ccccc3c3ccccc32)c1 ; c1cc(-c2ccc3c4c(cccc24)-c2ccccc2O3)cc(N(c2cccc(-n3c4ccccc4c4ccccc43)c2)c2ccccc2-c2cccc3ccccc23)c1 ; c1cc(-c2cc3c4c(cccc4c2)Oc2ccccc2-3)cc(N(c2cccc(-n3c4ccccc4c4ccccc43)c2)c2ccccc2-c2cccc3ccccc23)c1]

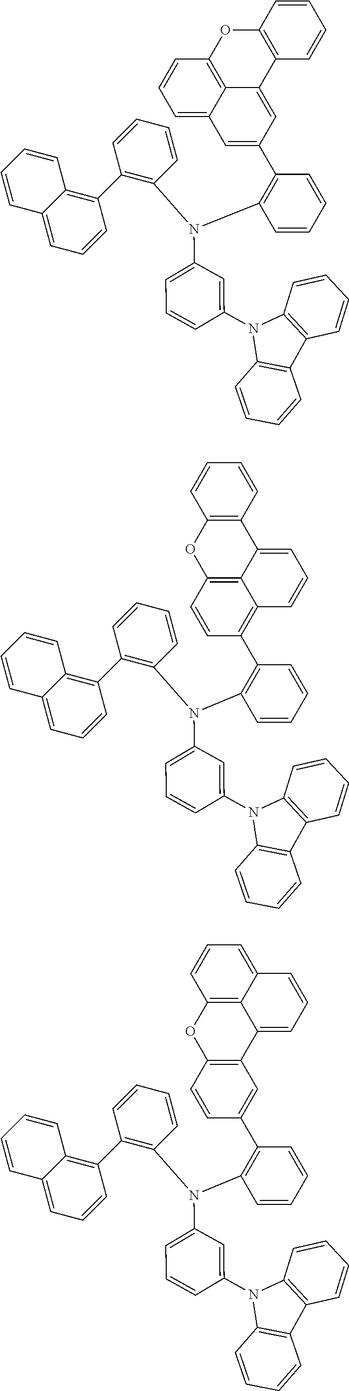

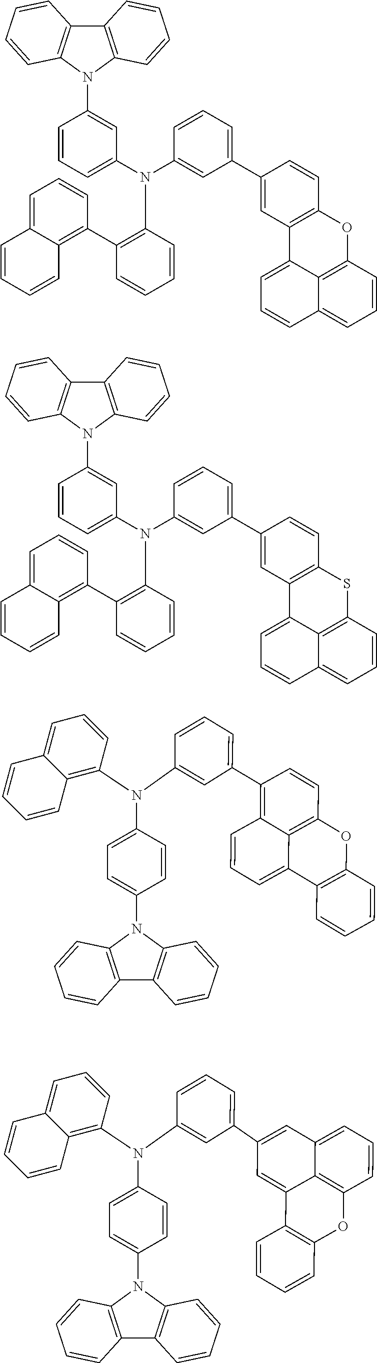 [SMILES: c1cc(-c2ccc3c(c2)-c2cccc4cccc(c24)O3)cc(N(c2cccc(-n3c4ccccc4c4ccccc43)c2)c2ccccc2-c2cccc3ccccc23)c1 ; c1cc(-c2ccc3c(c2)-c2cccc4cccc(c24)S3)cc(N(c2cccc(-n3c4ccccc4c4ccccc43)c2)c2ccccc2-c2cccc3ccccc23)c1 ; c1cc(-c2ccc3c4c(cccc24)-c2ccccc2O3)cc(N(c2ccc(-n3c4ccccc4c4ccccc43)cc2)c2cccc3ccccc23)c1 ; c1cc(-c2cc3c4c(cccc4c2)Oc2ccccc2-3)cc(N(c2ccc(-n3c4ccccc4c4ccccc43)cc2)c2cccc3ccccc23)c1]

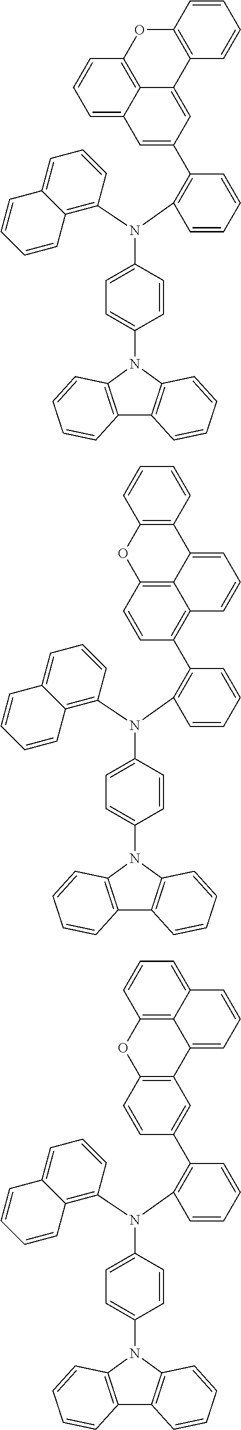

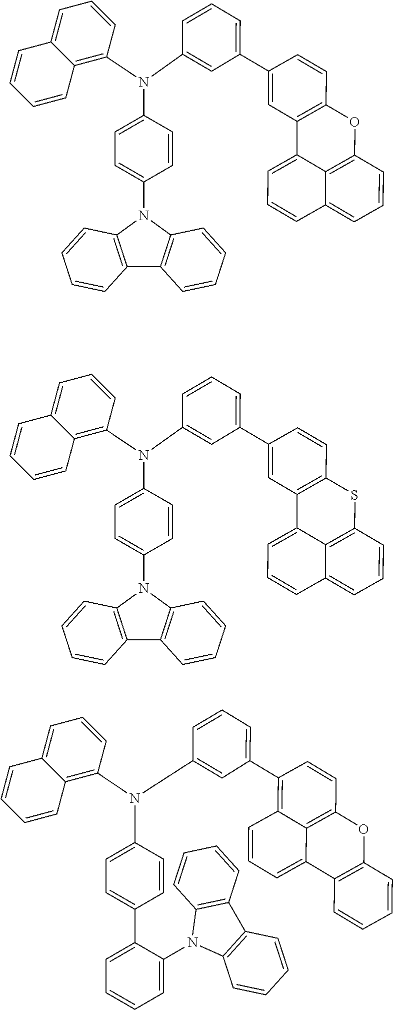 [SMILES: c1cc(-c2ccc3c(c2)-c2cccc4cccc(c24)O3)cc(N(c2ccc(-n3c4ccccc4c4ccccc43)cc2)c2cccc3ccccc23)c1 ; c1cc(-c2ccc3c(c2)-c2cccc4cccc(c24)S3)cc(N(c2ccc(-n3c4ccccc4c4ccccc43)cc2)c2cccc3ccccc23)c1 ; c1cc(-c2ccc3c4c(cccc24)-c2ccccc2O3)cc(N(c2ccc(-c3ccccc3-n3c4ccccc4c4ccccc43)cc2)c2cccc3ccccc23)c1]

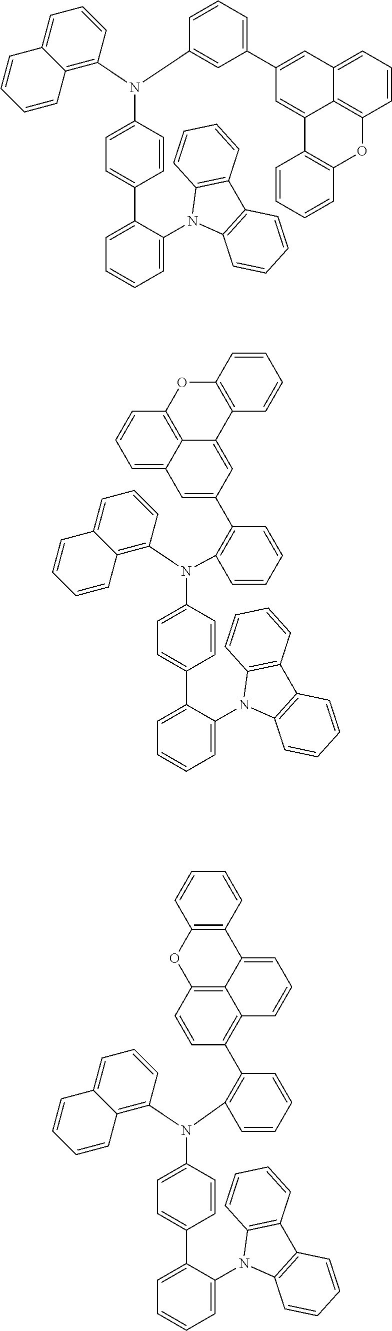 [SMILES: c1cc(-c2cc3c4c(cccc4c2)Oc2ccccc2-3)cc(N(c2ccc(-c3ccccc3-n3c4ccccc4c4ccccc43)cc2)c2cccc3ccccc23)c1 ; c1ccc2c(c1)Oc1cccc3cc(-c4ccccc4N(c4ccc(-c5ccccc5-n5c6ccccc6c6ccccc65)cc4)c4cccc5ccccc45)cc-2c13 ; c1ccc2c(c1)Oc1ccc(-c3ccccc3N(c3ccc(-c4ccccc4-n4c5ccccc5c5ccccc54)cc3)c3cccc4ccccc34)c3cccc-2c13]

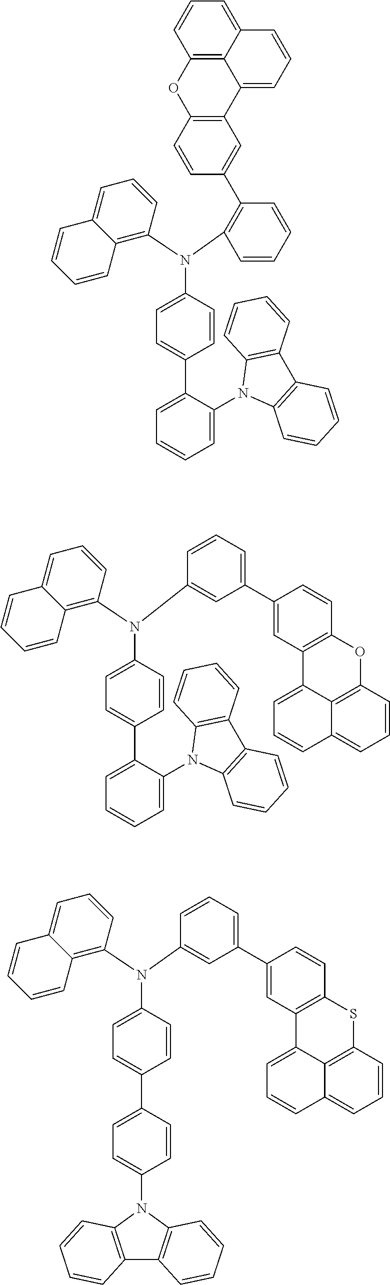 [SMILES: c1ccc(N(c2ccc(-c3ccccc3-n3c4ccccc4c4ccccc43)cc2)c2cccc3ccccc23)c(-c2ccc3c(c2)-c2cccc4cccc(c24)O3)c1 ; c1cc(-c2ccc3c(c2)-c2cccc4cccc(c24)O3)cc(N(c2ccc(-c3ccccc3-n3c4ccccc4c4ccccc43)cc2)c2cccc3ccccc23)c1 ; c1cc(-c2ccc3c(c2)-c2cccc4cccc(c24)S3)cc(N(c2ccc(-c3ccc(-n4c5ccccc5c5ccccc54)cc3)cc2)c2cccc3ccccc23)c1]

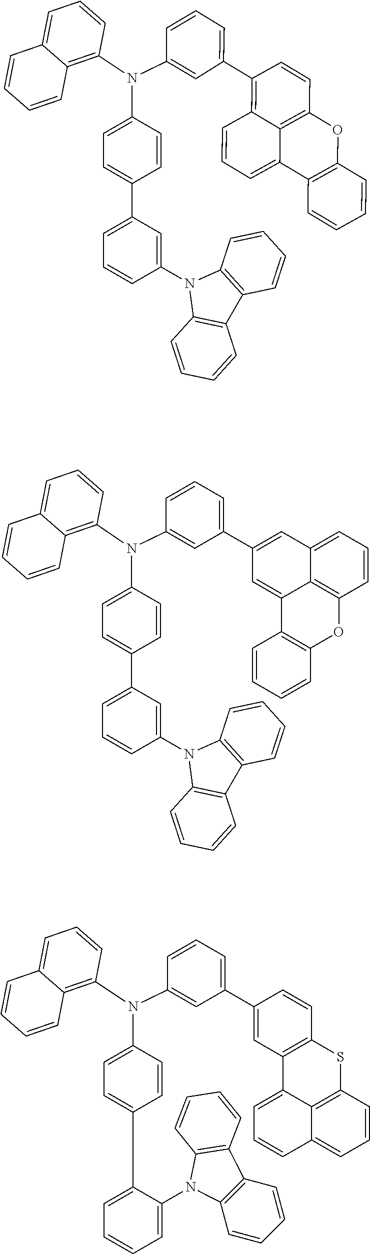 [SMILES: c1cc(-c2ccc3c4c(cccc24)-c2ccccc2O3)cc(N(c2ccc(-c3cccc(-n4c5ccccc5c5ccccc54)c3)cc2)c2cccc3ccccc23)c1 ; c1cc(-c2cc3c4c(cccc4c2)Oc2ccccc2-3)cc(N(c2ccc(-c3cccc(-n4c5ccccc5c5ccccc54)c3)cc2)c2cccc3ccccc23)c1 ; c1cc(-c2ccc3c(c2)-c2cccc4cccc(c24)S3)cc(N(c2ccc(-c3ccccc3-n3c4ccccc4c4ccccc43)cc2)c2cccc3ccccc23)c1]

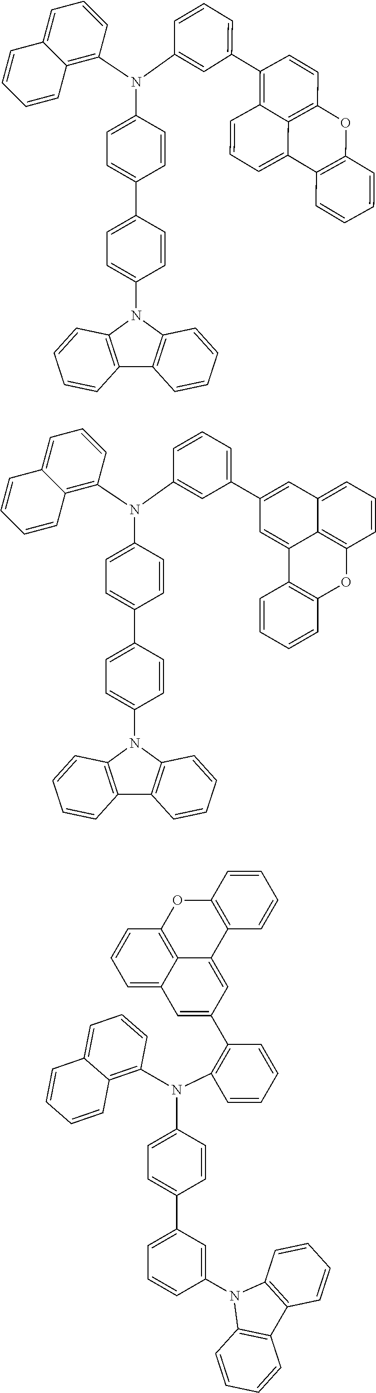

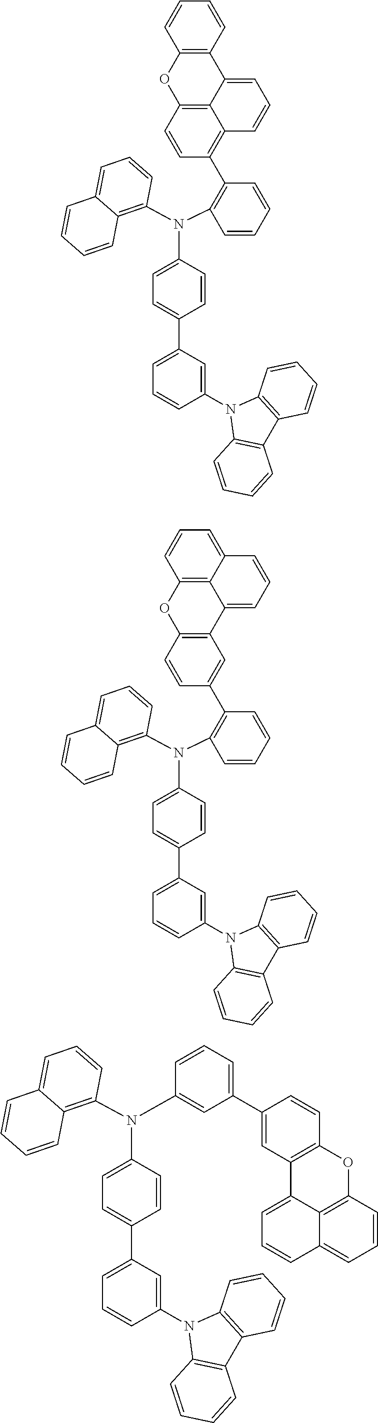

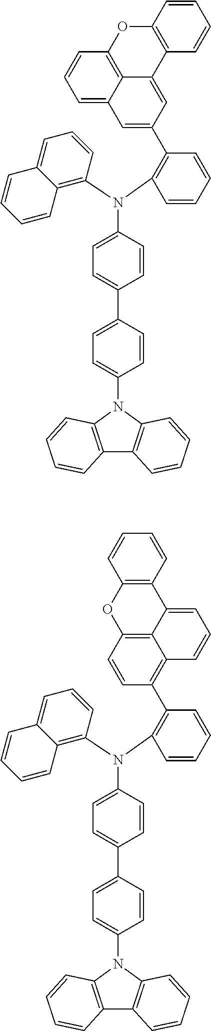 [SMILES: c1ccc2c(c1)Oc1cccc3cc(-c4ccccc4N(c4ccc(-c5ccc(-n6c7ccccc7c7ccccc76)cc5)cc4)c4cccc5ccccc45)cc-2c13 ; c1ccc2c(c1)Oc1ccc(-c3ccccc3N(c3ccc(-c4ccc(-n5c6ccccc6c6ccccc65)cc4)cc3)c3cccc4ccccc34)c3cccc-2c13]

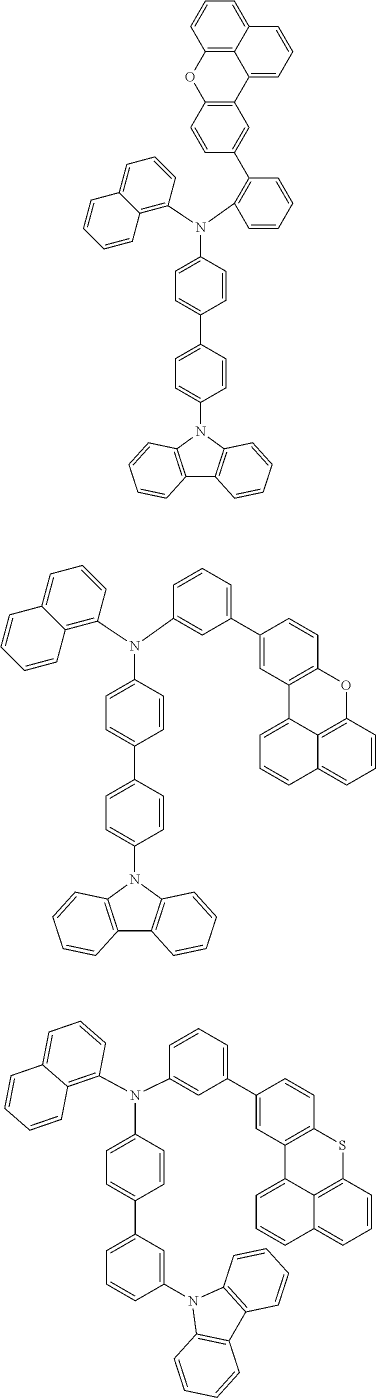 [SMILES: c1ccc(N(c2ccc(-c3ccc(-n4c5ccccc5c5ccccc54)cc3)cc2)c2cccc3ccccc23)c(-c2ccc3c(c2)-c2cccc4cccc(c24)O3)c1 ; c1cc(-c2ccc3c(c2)-c2cccc4cccc(c24)O3)cc(N(c2ccc(-c3ccc(-n4c5ccccc5c5ccccc54)cc3)cc2)c2cccc3ccccc23)c1 ; c1cc(-c2ccc3c(c2)-c2cccc4cccc(c24)S3)cc(N(c2ccc(-c3cccc(-n4c5ccccc5c5ccccc54)c3)cc2)c2cccc3ccccc23)c1]

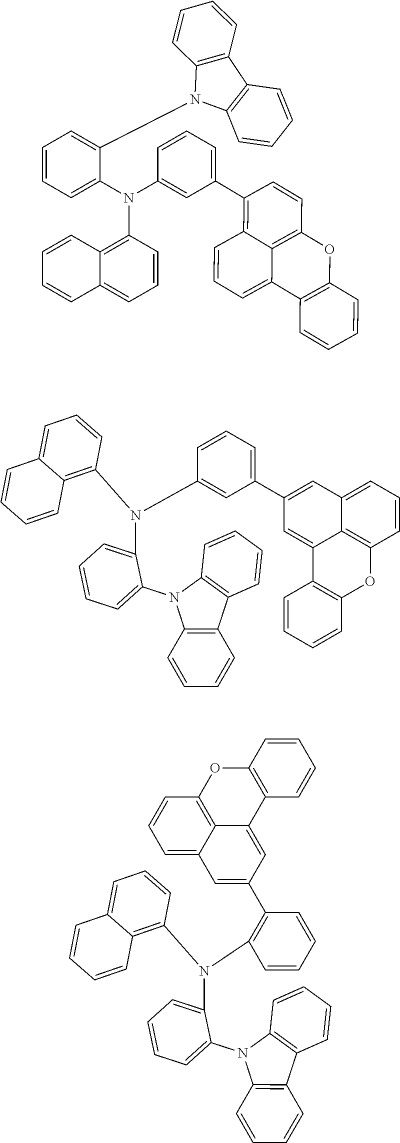 [SMILES: c1cc(-c2ccc3c4c(cccc24)-c2ccccc2O3)cc(N(c2ccccc2-n2c3ccccc3c3ccccc32)c2cccc3ccccc23)c1 ; c1cc(-c2cc3c4c(cccc4c2)Oc2ccccc2-3)cc(N(c2ccccc2-n2c3ccccc3c3ccccc32)c2cccc3ccccc23)c1 ; c1ccc2c(c1)Oc1cccc3cc(-c4ccccc4N(c4ccccc4-n4c5ccccc5c5ccccc54)c4cccc5ccccc45)cc-2c13]

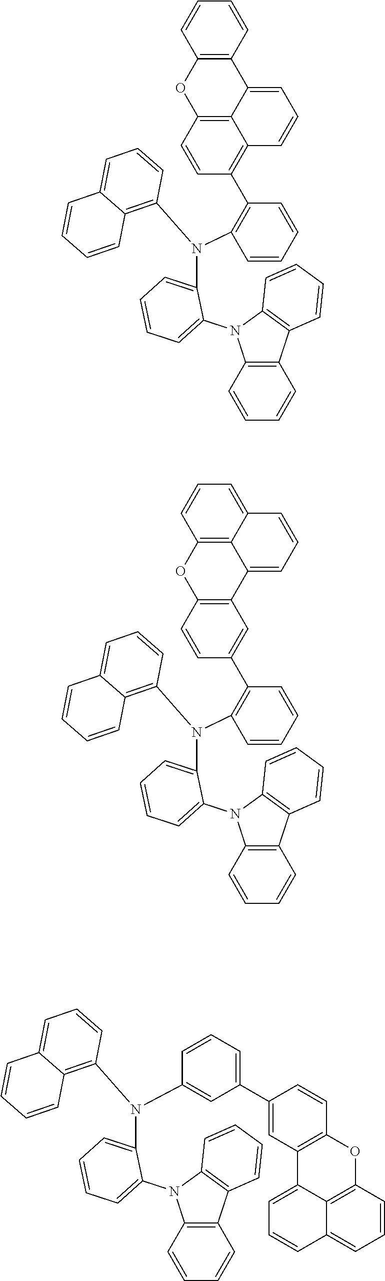 [SMILES: c1ccc2c(c1)Oc1ccc(-c3ccccc3N(c3ccccc3-n3c4ccccc4c4ccccc43)c3cccc4ccccc34)c3cccc-2c13 ; c1ccc(N(c2ccccc2-n2c3ccccc3c3ccccc32)c2cccc3ccccc23)c(-c2ccc3c(c2)-c2cccc4cccc(c24)O3)c1 ; c1cc(-c2ccc3c(c2)-c2cccc4cccc(c24)O3)cc(N(c2ccccc2-n2c3ccccc3c3ccccc32)c2cccc3ccccc23)c1]

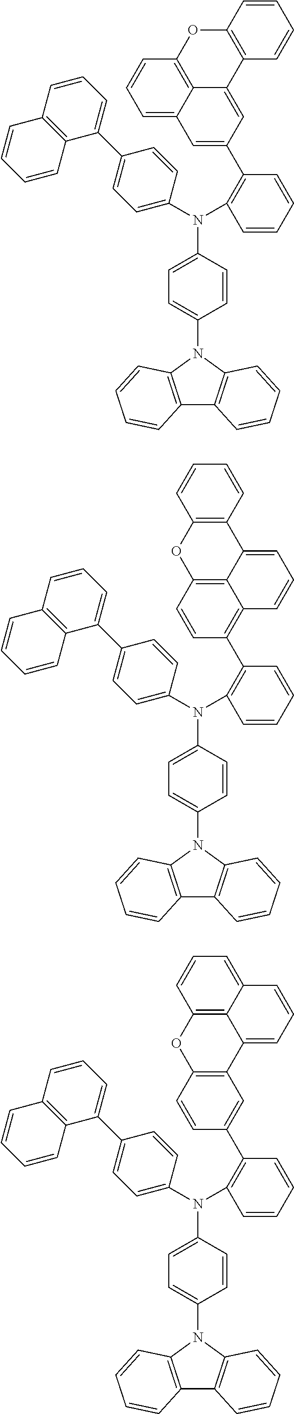

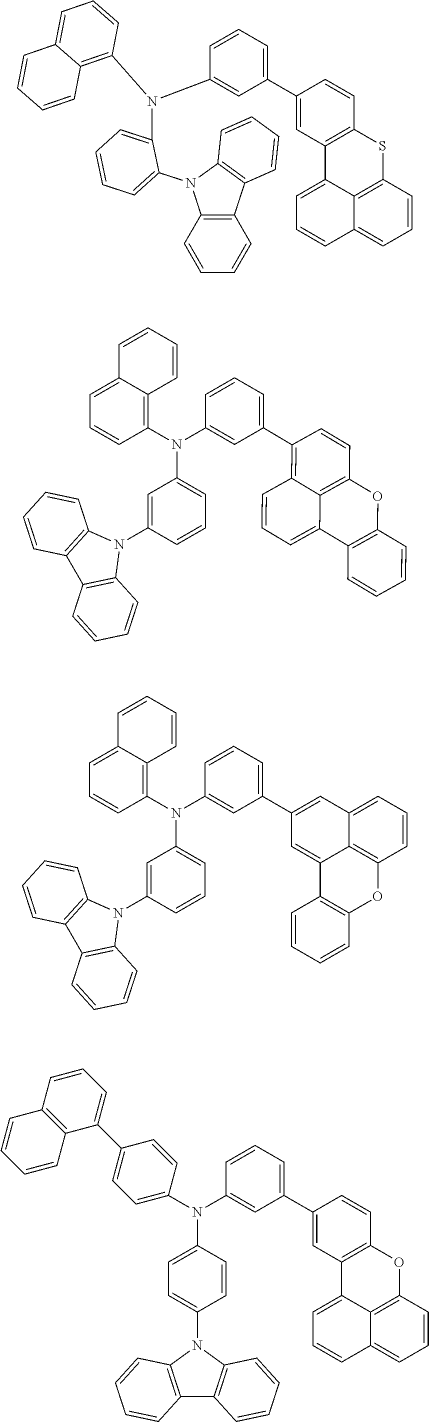 [SMILES: c1cc(-c2ccc3c(c2)-c2cccc4cccc(c24)S3)cc(N(c2ccccc2-n2c3ccccc3c3ccccc32)c2cccc3ccccc23)c1 ; c1cc(-c2ccc3c4c(cccc24)-c2ccccc2O3)cc(N(c2cccc(-n3c4ccccc4c4ccccc43)c2)c2cccc3ccccc23)c1 ; c1cc(-c2cc3c4c(cccc4c2)Oc2ccccc2-3)cc(N(c2cccc(-n3c4ccccc4c4ccccc43)c2)c2cccc3ccccc23)c1 ; c1cc(-c2ccc3c(c2)-c2cccc4cccc(c24)O3)cc(N(c2ccc(-c3cccc4ccccc34)cc2)c2ccc(-n3c4ccccc4c4ccccc43)cc2)c1]

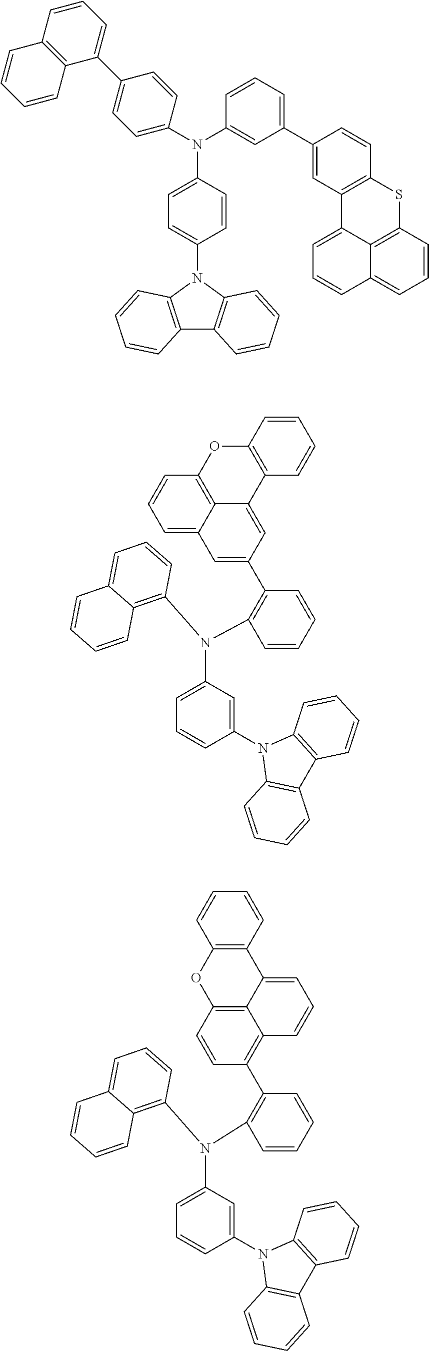 [SMILES: c1cc(-c2ccc3c(c2)-c2cccc4cccc(c24)S3)cc(N(c2ccc(-c3cccc4ccccc34)cc2)c2ccc(-n3c4ccccc4c4ccccc43)cc2)c1 ; c1cc(N(c2ccccc2-c2cc3c4c(cccc4c2)Oc2ccccc2-3)c2cccc3ccccc23)cc(-n2c3ccccc3c3ccccc32)c1 ; c1cc(N(c2ccccc2-c2ccc3c4c(cccc24)-c2ccccc2O3)c2cccc3ccccc23)cc(-n2c3ccccc3c3ccccc32)c1]

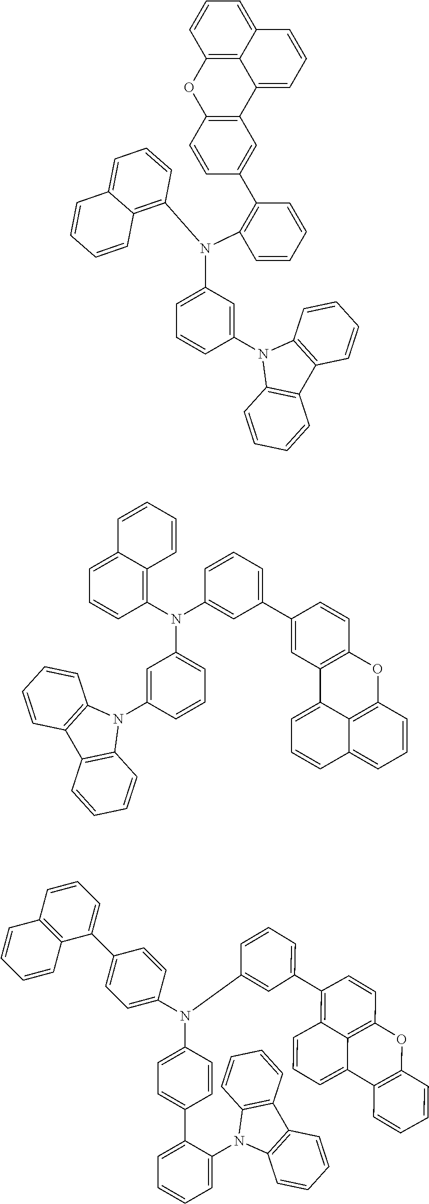 [SMILES: c1cc(N(c2ccccc2-c2ccc3c(c2)-c2cccc4cccc(c24)O3)c2cccc3ccccc23)cc(-n2c3ccccc3c3ccccc32)c1 ; c1cc(-c2ccc3c(c2)-c2cccc4cccc(c24)O3)cc(N(c2cccc(-n3c4ccccc4c4ccccc43)c2)c2cccc3ccccc23)c1 ; c1cc(-c2ccc3c4c(cccc24)-c2ccccc2O3)cc(N(c2ccc(-c3ccccc3-n3c4ccccc4c4ccccc43)cc2)c2ccc(-c3cccc4ccccc34)cc2)c1]

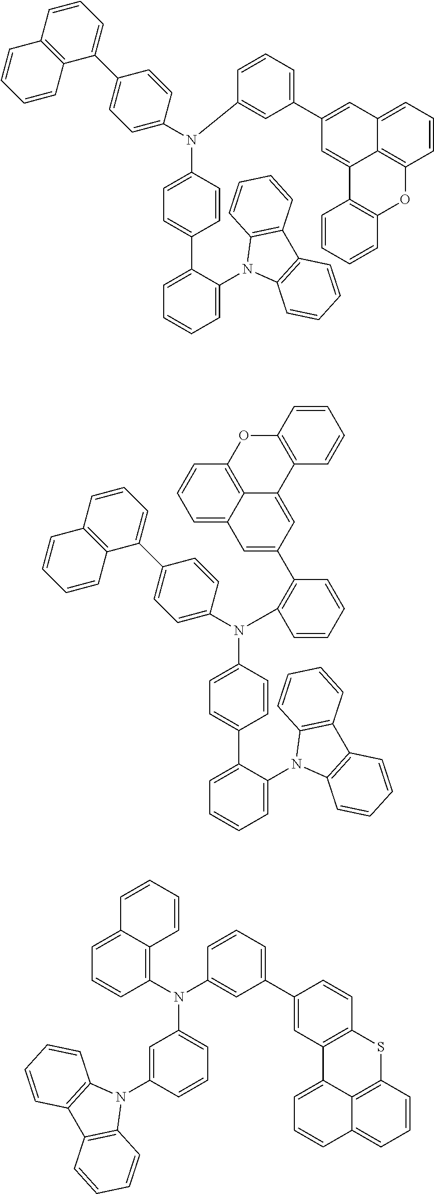 [SMILES: c1cc(-c2cc3c4c(cccc4c2)Oc2ccccc2-3)cc(N(c2ccc(-c3ccccc3-n3c4ccccc4c4ccccc43)cc2)c2ccc(-c3cccc4ccccc34)cc2)c1 ; c1ccc2c(c1)Oc1cccc3cc(-c4ccccc4N(c4ccc(-c5ccccc5-n5c6ccccc6c6ccccc65)cc4)c4ccc(-c5cccc6ccccc56)cc4)cc-2c13 ; c1cc(-c2ccc3c(c2)-c2cccc4cccc(c24)S3)cc(N(c2cccc(-n3c4ccccc4c4ccccc43)c2)c2cccc3ccccc23)c1]

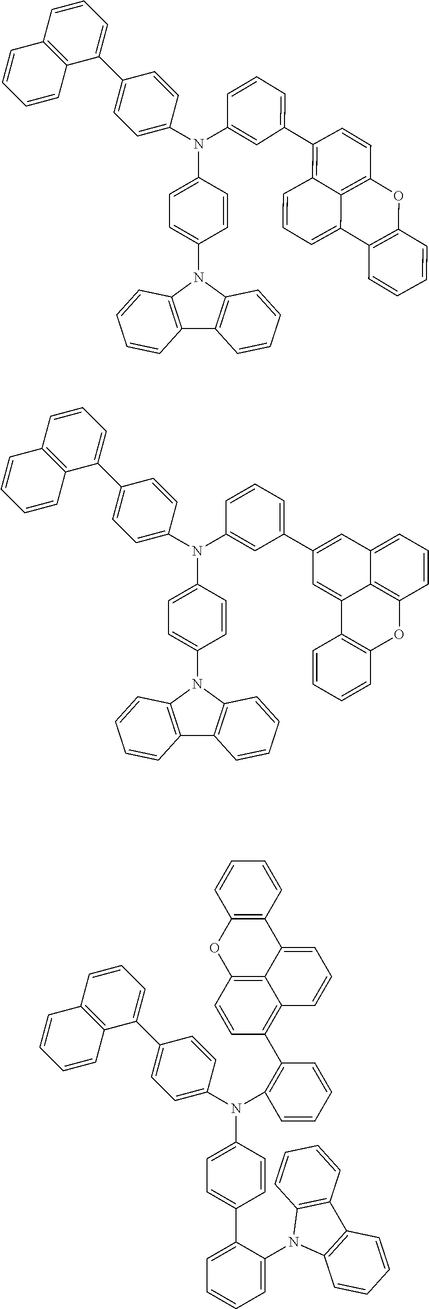 [SMILES: c1cc(-c2ccc3c4c(cccc24)-c2ccccc2O3)cc(N(c2ccc(-c3cccc4ccccc34)cc2)c2ccc(-n3c4ccccc4c4ccccc43)cc2)c1 ; c1cc(-c2cc3c4c(cccc4c2)Oc2ccccc2-3)cc(N(c2ccc(-c3cccc4ccccc34)cc2)c2ccc(-n3c4ccccc4c4ccccc43)cc2)c1 ; c1ccc2c(c1)Oc1ccc(-c3ccccc3N(c3ccc(-c4ccccc4-n4c5ccccc5c5ccccc54)cc3)c3ccc(-c4cccc5ccccc45)cc3)c3cccc-2c13]

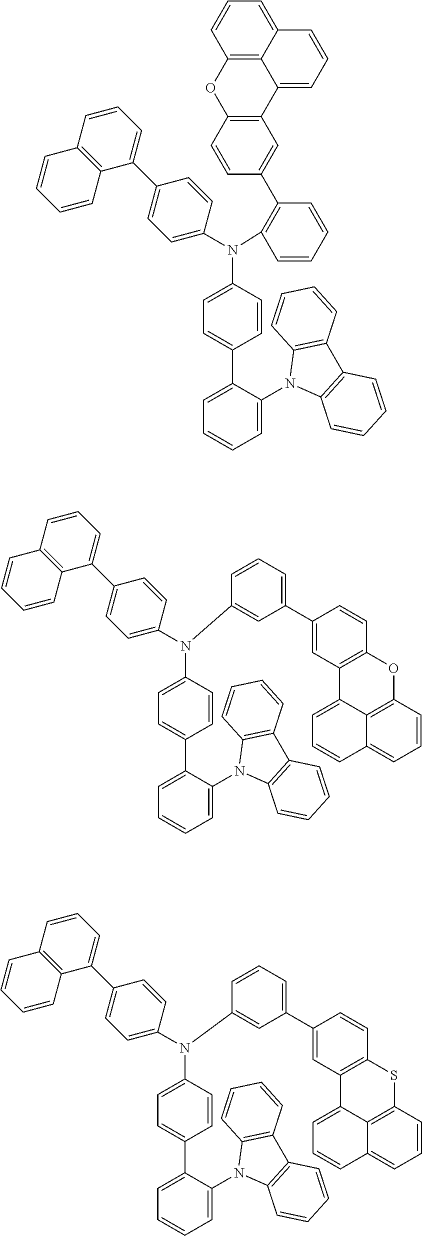 [SMILES: c1ccc(N(c2ccc(-c3ccccc3-n3c4ccccc4c4ccccc43)cc2)c2ccc(-c3cccc4ccccc34)cc2)c(-c2ccc3c(c2)-c2cccc4cccc(c24)O3)c1 ; c1cc(-c2ccc3c(c2)-c2cccc4cccc(c24)O3)cc(N(c2ccc(-c3ccccc3-n3c4ccccc4c4ccccc43)cc2)c2ccc(-c3cccc4ccccc34)cc2)c1 ; c1cc(-c2ccc3c(c2)-c2cccc4cccc(c24)S3)cc(N(c2ccc(-c3ccccc3-n3c4ccccc4c4ccccc43)cc2)c2ccc(-c3cccc4ccccc34)cc2)c1]

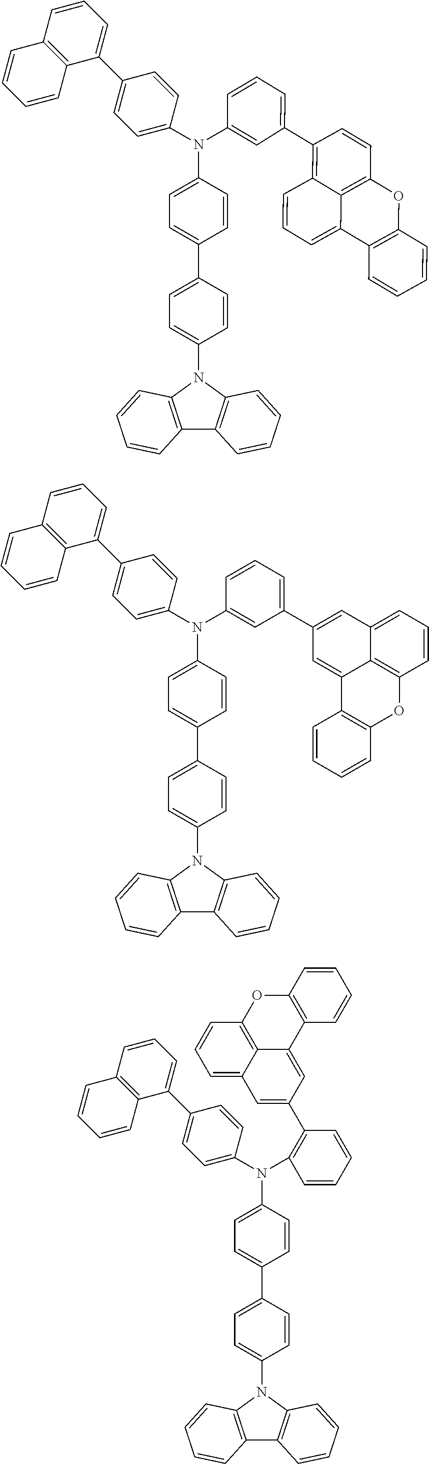 [SMILES: c1cc(-c2ccc3c4c(cccc24)-c2ccccc2O3)cc(N(c2ccc(-c3ccc(-n4c5ccccc5c5ccccc54)cc3)cc2)c2ccc(-c3cccc4ccccc34)cc2)c1 ; c1cc(-c2cc3c4c(cccc4c2)Oc2ccccc2-3)cc(N(c2ccc(-c3ccc(-n4c5ccccc5c5ccccc54)cc3)cc2)c2ccc(-c3cccc4ccccc34)cc2)c1 ; c1ccc2c(c1)Oc1cccc3cc(-c4ccccc4N(c4ccc(-c5ccc(-n6c7ccccc7c7ccccc76)cc5)cc4)c4ccc(-c5cccc6ccccc56)cc4)cc-2c13]

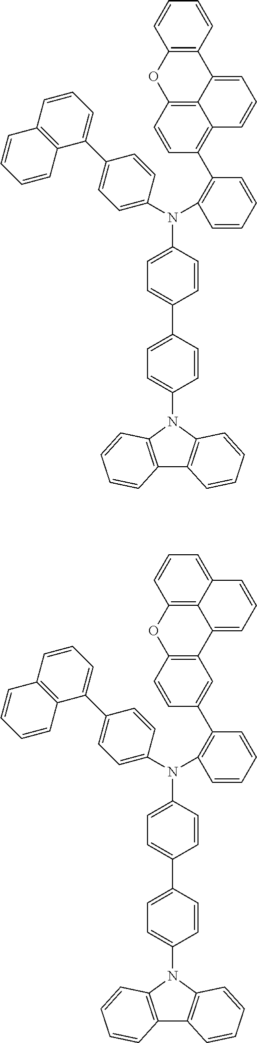 [SMILES: c1ccc2c(c1)Oc1ccc(-c3ccccc3N(c3ccc(-c4ccc(-n5c6ccccc6c6ccccc65)cc4)cc3)c3ccc(-c4cccc5ccccc45)cc3)c3cccc-2c13 ; c1ccc(N(c2ccc(-c3ccc(-n4c5ccccc5c5ccccc54)cc3)cc2)c2ccc(-c3cccc4ccccc34)cc2)c(-c2ccc3c(c2)-c2cccc4cccc(c24)O3)c1]

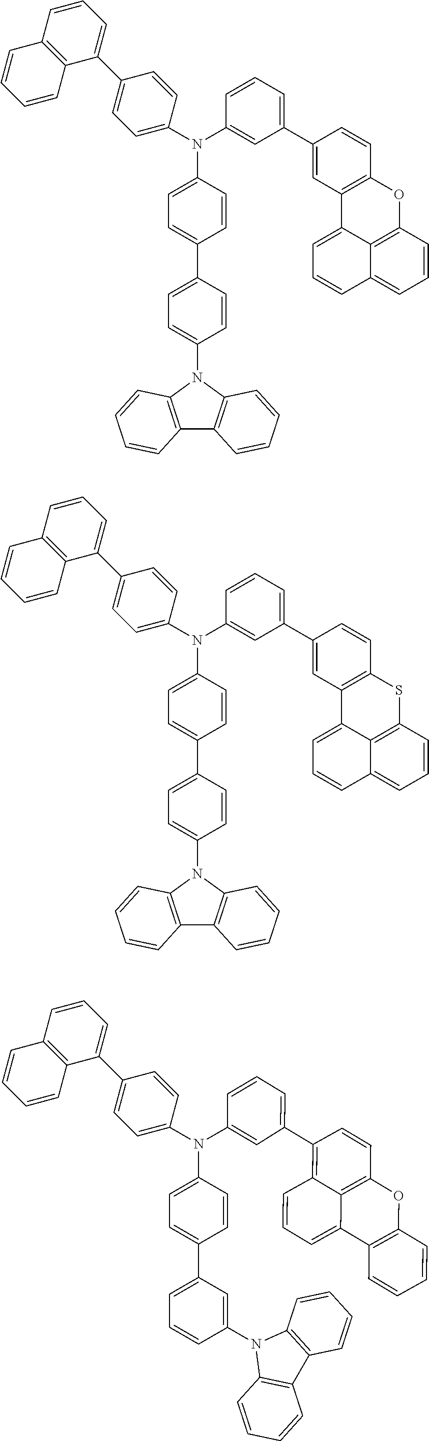 [SMILES: c1cc(-c2ccc3c(c2)-c2cccc4cccc(c24)O3)cc(N(c2ccc(-c3ccc(-n4c5ccccc5c5ccccc54)cc3)cc2)c2ccc(-c3cccc4ccccc34)cc2)c1 ; c1cc(-c2ccc3c(c2)-c2cccc4cccc(c24)S3)cc(N(c2ccc(-c3ccc(-n4c5ccccc5c5ccccc54)cc3)cc2)c2ccc(-c3cccc4ccccc34)cc2)c1 ; c1cc(-c2ccc3c4c(cccc24)-c2ccccc2O3)cc(N(c2ccc(-c3cccc(-n4c5ccccc5c5ccccc54)c3)cc2)c2ccc(-c3cccc4ccccc34)cc2)c1]

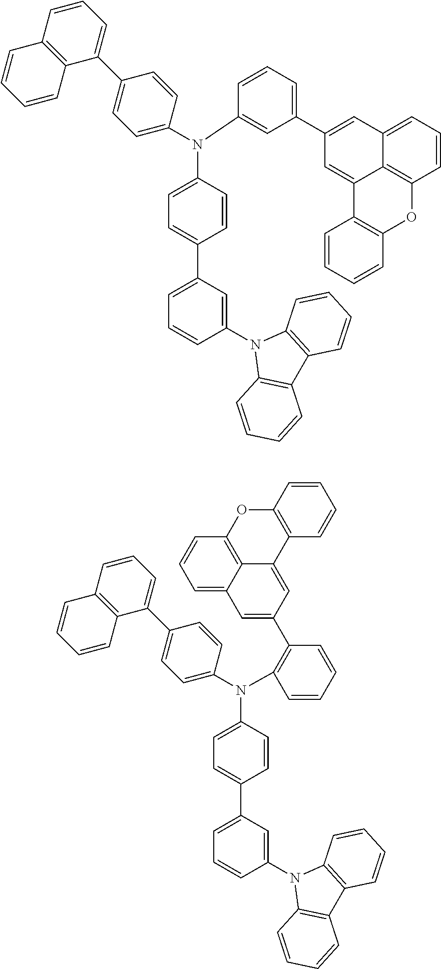

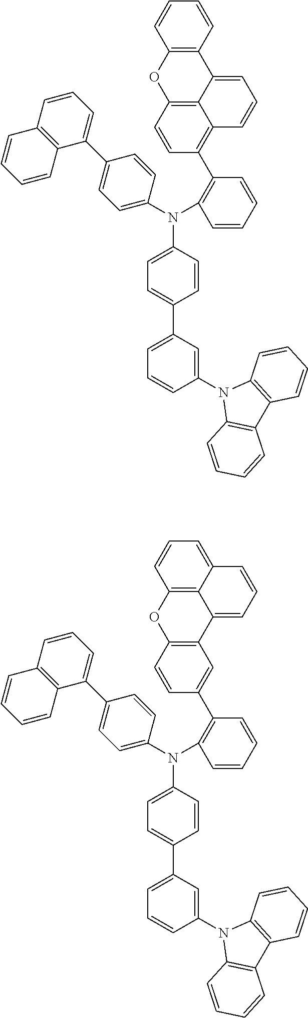 [SMILES: c1cc(-c2ccc(N(c3ccc(-c4cccc5ccccc45)cc3)c3ccccc3-c3ccc4c5c(cccc35)-c3ccccc3O4)cc2)cc(-n2c3ccccc3c3ccccc32)c1 ; c1cc(-c2ccc(N(c3ccc(-c4cccc5ccccc45)cc3)c3ccccc3-c3ccc4c(c3)-c3cccc5cccc(c35)O4)cc2)cc(-n2c3ccccc3c3ccccc32)c1]

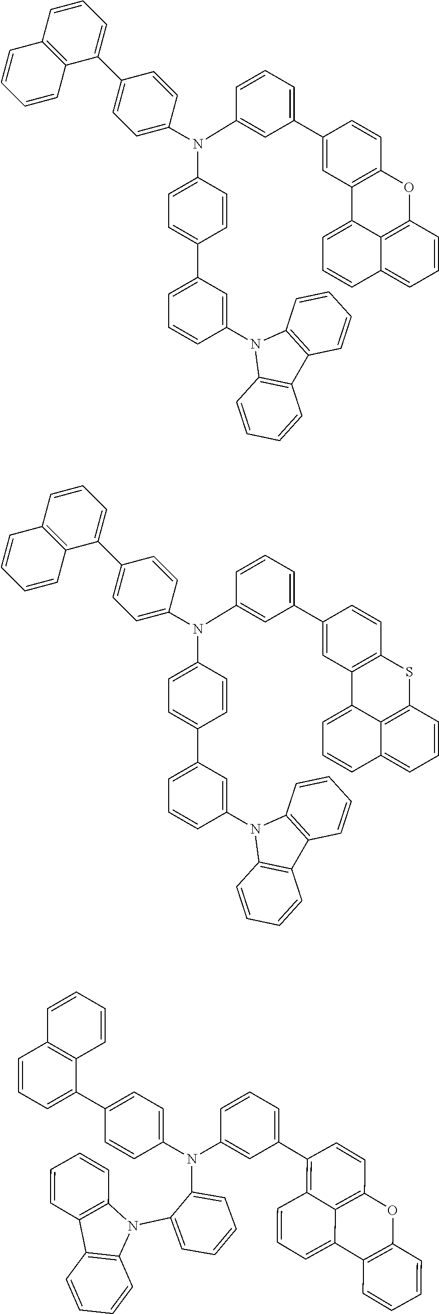 [SMILES: c1cc(-c2ccc3c(c2)-c2cccc4cccc(c24)O3)cc(N(c2ccc(-c3cccc(-n4c5ccccc5c5ccccc54)c3)cc2)c2ccc(-c3cccc4ccccc34)cc2)c1 ; c1cc(-c2ccc3c(c2)-c2cccc4cccc(c24)S3)cc(N(c2ccc(-c3cccc(-n4c5ccccc5c5ccccc54)c3)cc2)c2ccc(-c3cccc4ccccc34)cc2)c1 ; c1cc(-c2ccc3c4c(cccc24)-c2ccccc2O3)cc(N(c2ccc(-c3cccc4ccccc34)cc2)c2ccccc2-n2c3ccccc3c3ccccc32)c1]

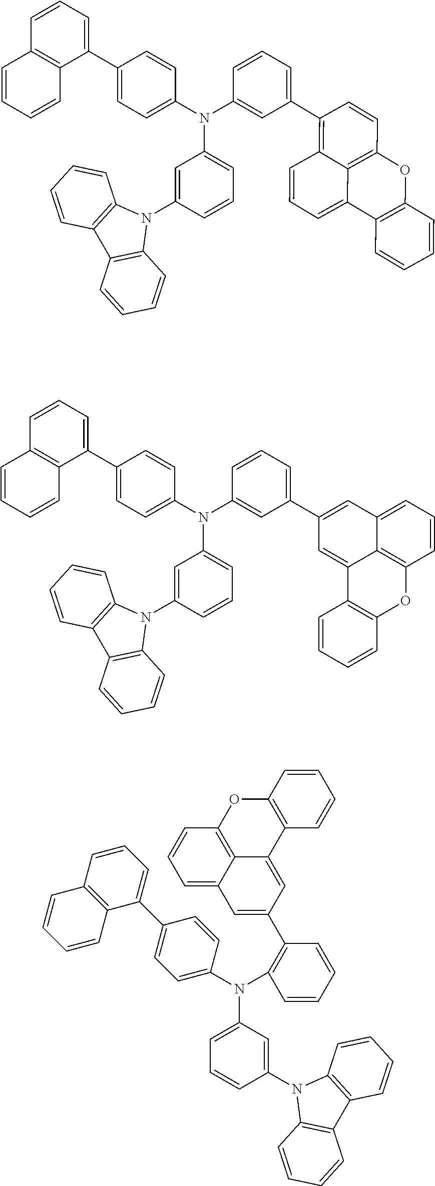 [SMILES: c1cc(-c2ccc3c4c(cccc24)-c2ccccc2O3)cc(N(c2ccc(-c3cccc4ccccc34)cc2)c2cccc(-n3c4ccccc4c4ccccc43)c2)c1 ; c1cc(-c2cc3c4c(cccc4c2)Oc2ccccc2-3)cc(N(c2ccc(-c3cccc4ccccc34)cc2)c2cccc(-n3c4ccccc4c4ccccc43)c2)c1 ; c1cc(N(c2ccc(-c3cccc4ccccc34)cc2)c2ccccc2-c2cc3c4c(cccc4c2)Oc2ccccc2-3)cc(-n2c3ccccc3c3ccccc32)c1]

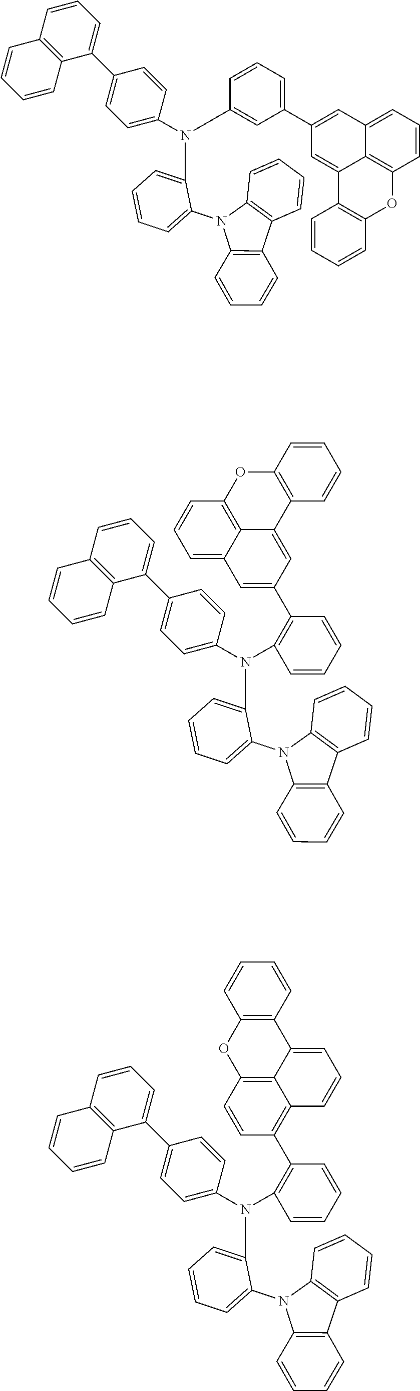 [SMILES: c1cc(-c2cc3c4c(cccc4c2)Oc2ccccc2-3)cc(N(c2ccc(-c3cccc4ccccc34)cc2)c2ccccc2-n2c3ccccc3c3ccccc32)c1 ; c1ccc2c(c1)Oc1cccc3cc(-c4ccccc4N(c4ccc(-c5cccc6ccccc56)cc4)c4ccccc4-n4c5ccccc5c5ccccc54)cc-2c13 ; c1ccc2c(c1)Oc1ccc(-c3ccccc3N(c3ccc(-c4cccc5ccccc45)cc3)c3ccccc3-n3c4ccccc4c4ccccc43)c3cccc-2c13]

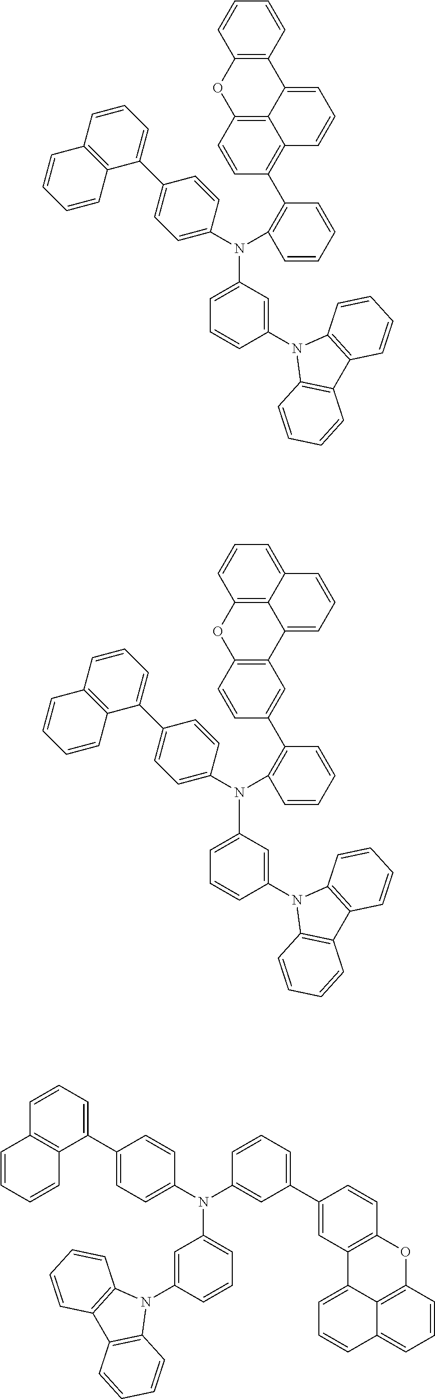 [SMILES: c1cc(N(c2ccc(-c3cccc4ccccc34)cc2)c2ccccc2-c2ccc3c4c(cccc24)-c2ccccc2O3)cc(-n2c3ccccc3c3ccccc32)c1 ; c1cc(N(c2ccc(-c3cccc4ccccc34)cc2)c2ccccc2-c2ccc3c(c2)-c2cccc4cccc(c24)O3)cc(-n2c3ccccc3c3ccccc32)c1 ; c1cc(-c2ccc3c(c2)-c2cccc4cccc(c24)O3)cc(N(c2ccc(-c3cccc4ccccc34)cc2)c2cccc(-n3c4ccccc4c4ccccc43)c2)c1]

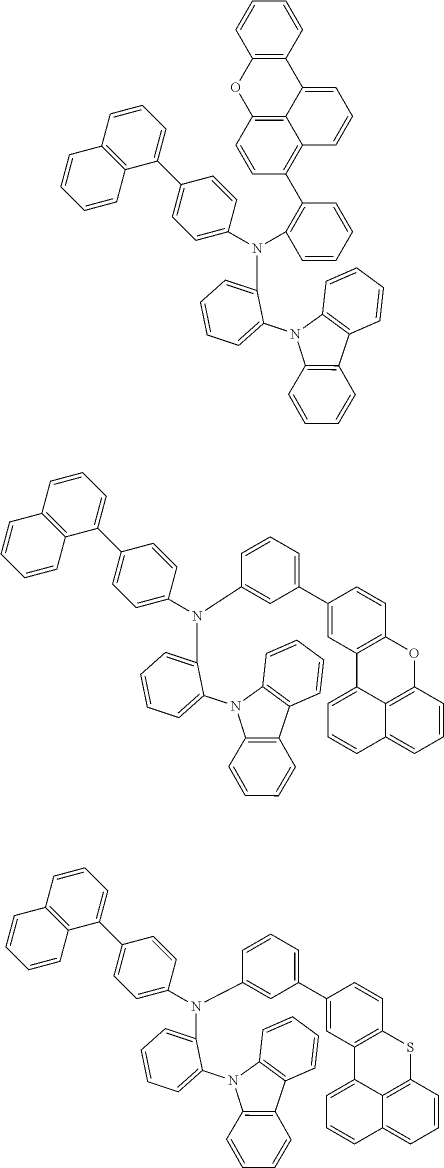 [SMILES: c1ccc2c(c1)Oc1ccc(-c3ccccc3N(c3ccc(-c4cccc5ccccc45)cc3)c3ccccc3-n3c4ccccc4c4ccccc43)c3cccc-2c13 ; c1cc(-c2ccc3c(c2)-c2cccc4cccc(c24)O3)cc(N(c2ccc(-c3cccc4ccccc34)cc2)c2ccccc2-n2c3ccccc3c3ccccc32)c1 ; c1cc(-c2ccc3c(c2)-c2cccc4cccc(c24)S3)cc(N(c2ccc(-c3cccc4ccccc34)cc2)c2ccccc2-n2c3ccccc3c3ccccc32)c1]

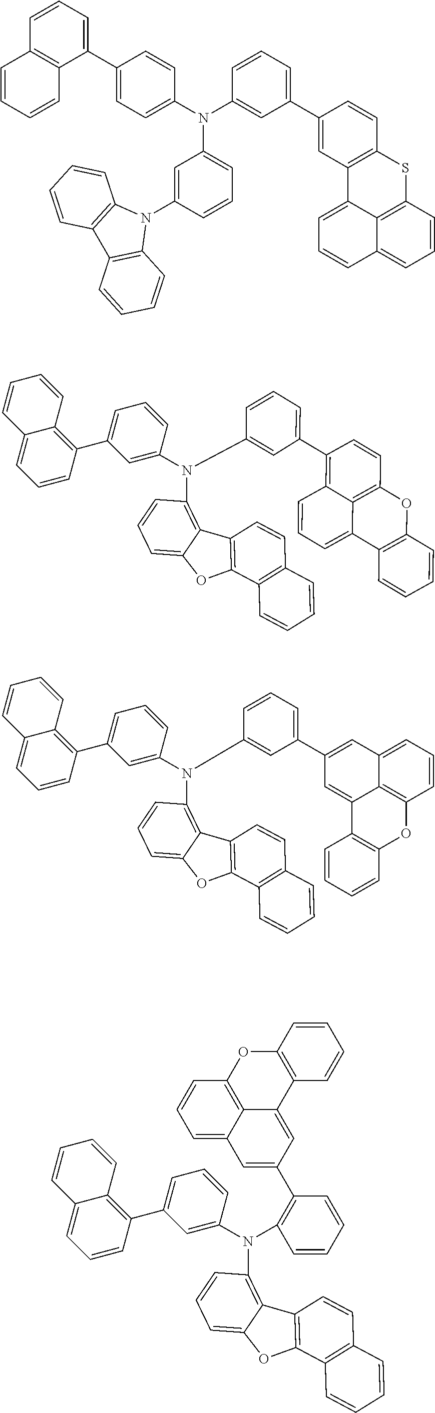 [SMILES: c1cc(-c2ccc3c(c2)-c2cccc4cccc(c24)S3)cc(N(c2ccc(-c3cccc4ccccc34)cc2)c2cccc(-n3c4ccccc4c4ccccc43)c2)c1 ; c1cc(-c2cccc3ccccc23)cc(N(c2cccc(-c3ccc4c5c(cccc35)-c3ccccc3O4)c2)c2cccc3oc4c5ccccc5ccc4c23)c1 ; c1cc(-c2cc3c4c(cccc4c2)Oc2ccccc2-3)cc(N(c2cccc(-c3cccc4ccccc34)c2)c2cccc3oc4c5ccccc5ccc4c23)c1 ; c1cc(-c2cccc3ccccc23)cc(N(c2ccccc2-c2cc3c4c(cccc4c2)Oc2ccccc2-3)c2cccc3oc4c5ccccc5ccc4c23)c1]

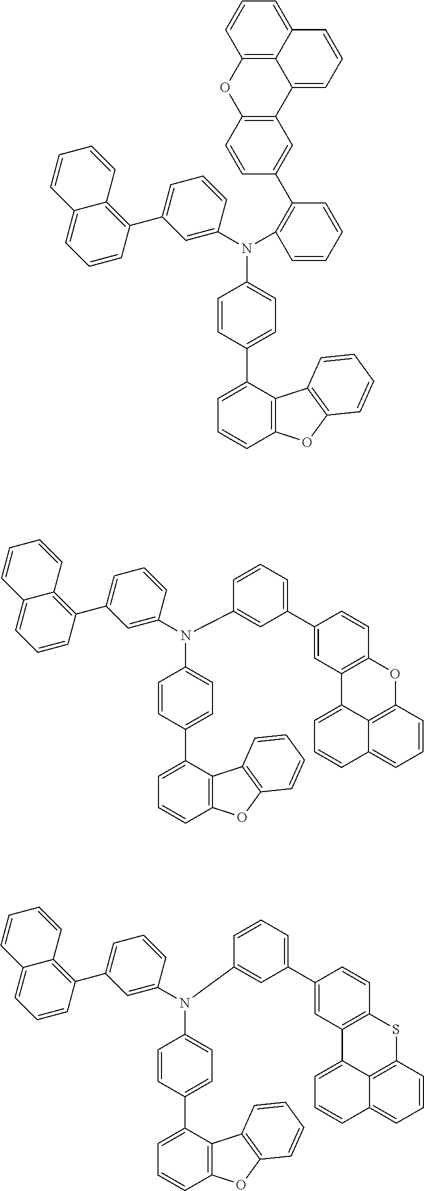 [SMILES: c1cc(-c2cccc3ccccc23)cc(N(c2ccc(-c3cccc4oc5ccccc5c34)cc2)c2ccccc2-c2ccc3c(c2)-c2cccc4cccc(c24)O3)c1 ; c1cc(-c2ccc3c(c2)-c2cccc4cccc(c24)O3)cc(N(c2ccc(-c3cccc4oc5ccccc5c34)cc2)c2cccc(-c3cccc4ccccc34)c2)c1 ; c1cc(-c2ccc3c(c2)-c2cccc4cccc(c24)S3)cc(N(c2ccc(-c3cccc4oc5ccccc5c34)cc2)c2cccc(-c3cccc4ccccc34)c2)c1]

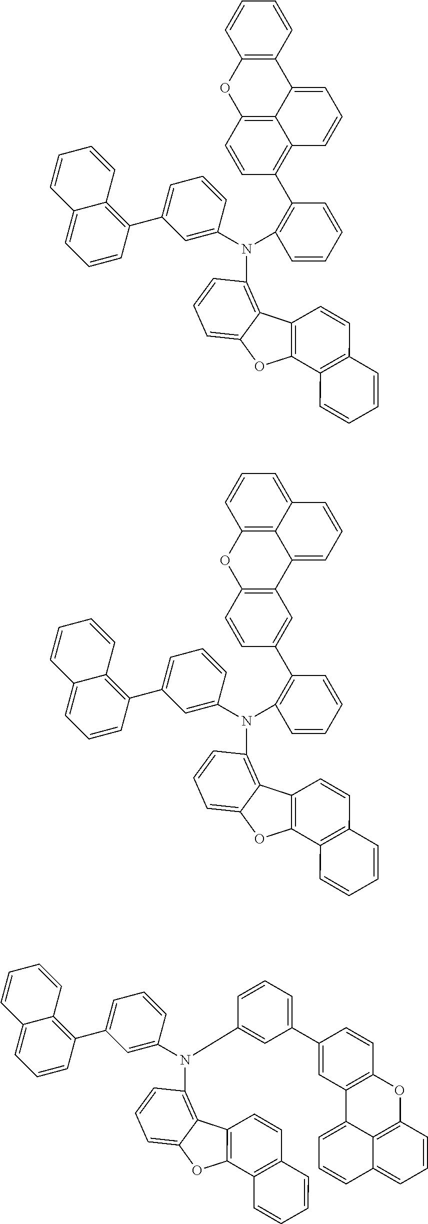 [SMILES: c1cc(-c2cccc3ccccc23)cc(N(c2ccccc2-c2ccc3c4c(cccc24)-c2ccccc2O3)c2cccc3oc4c5ccccc5ccc4c23)c1 ; c1cc(-c2cccc3ccccc23)cc(N(c2ccccc2-c2ccc3c(c2)-c2cccc4cccc(c24)O3)c2cccc3oc4c5ccccc5ccc4c23)c1 ; c1cc(-c2ccc3c(c2)-c2cccc4cccc(c24)O3)cc(N(c2cccc(-c3cccc4ccccc34)c2)c2cccc3oc4c5ccccc5ccc4c23)c1]

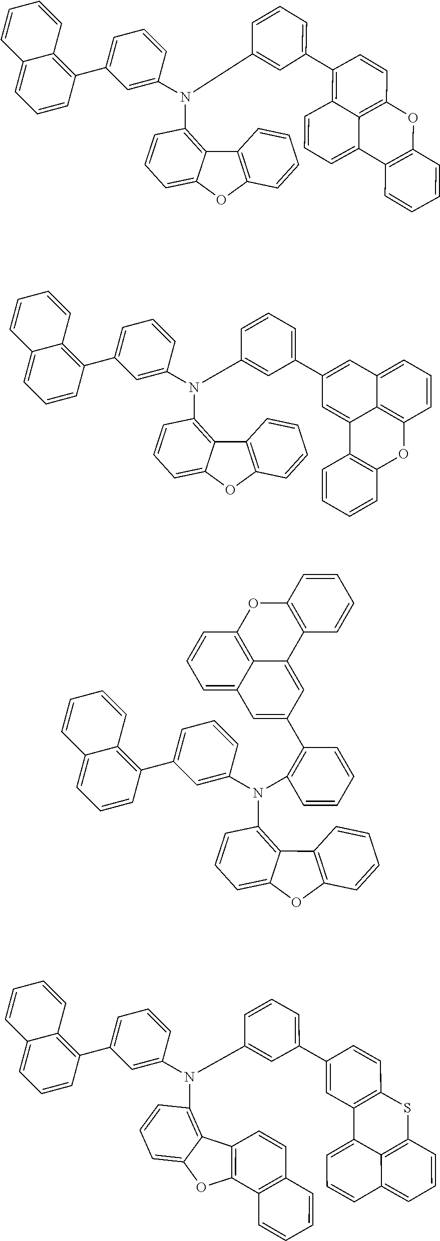 [SMILES: c1cc(-c2cccc3ccccc23)cc(N(c2cccc(-c3ccc4c5c(cccc35)-c3ccccc3O4)c2)c2cccc3oc4ccccc4c23)c1 ; c1cc(-c2cc3c4c(cccc4c2)Oc2ccccc2-3)cc(N(c2cccc(-c3cccc4ccccc34)c2)c2cccc3oc4ccccc4c23)c1 ; c1cc(-c2cccc3ccccc23)cc(N(c2ccccc2-c2cc3c4c(cccc4c2)Oc2ccccc2-3)c2cccc3oc4ccccc4c23)c1 ; c1cc(-c2ccc3c(c2)-c2cccc4cccc(c24)S3)cc(N(c2cccc(-c3cccc4ccccc34)c2)c2cccc3oc4c5ccccc5ccc4c23)c1]

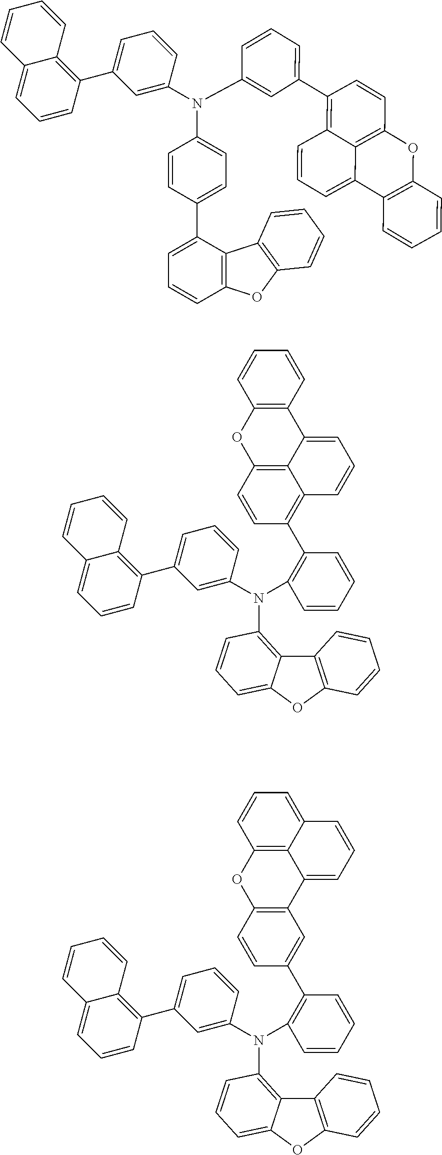 [SMILES: c1cc(-c2cccc3ccccc23)cc(N(c2ccc(-c3cccc4oc5ccccc5c34)cc2)c2cccc(-c3ccc4c5c(cccc35)-c3ccccc3O4)c2)c1 ; c1cc(-c2cccc3ccccc23)cc(N(c2ccccc2-c2ccc3c4c(cccc24)-c2ccccc2O3)c2cccc3oc4ccccc4c23)c1 ; c1cc(-c2cccc3ccccc23)cc(N(c2ccccc2-c2ccc3c(c2)-c2cccc4cccc(c24)O3)c2cccc3oc4ccccc4c23)c1]

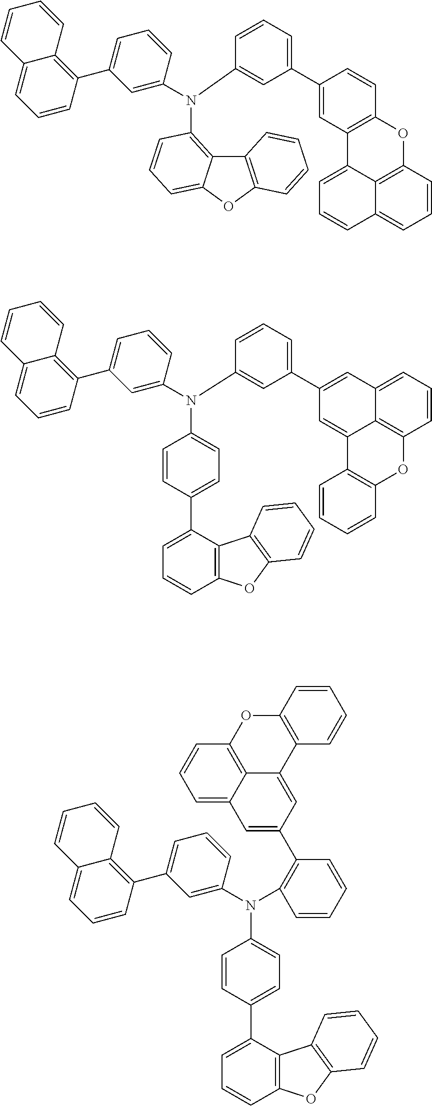 [SMILES: c1cc(-c2ccc3c(c2)-c2cccc4cccc(c24)O3)cc(N(c2cccc(-c3cccc4ccccc34)c2)c2cccc3oc4ccccc4c23)c1 ; c1cc(-c2cc3c4c(cccc4c2)Oc2ccccc2-3)cc(N(c2ccc(-c3cccc4oc5ccccc5c34)cc2)c2cccc(-c3cccc4ccccc34)c2)c1 ; c1cc(-c2cccc3ccccc23)cc(N(c2ccc(-c3cccc4oc5ccccc5c34)cc2)c2ccccc2-c2cc3c4c(cccc4c2)Oc2ccccc2-3)c1]

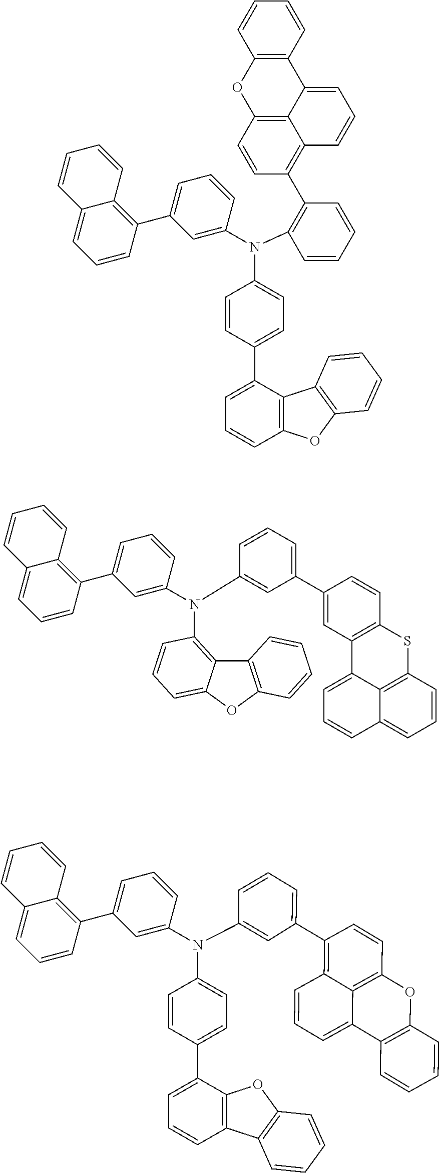 [SMILES: c1cc(-c2cccc3ccccc23)cc(N(c2ccc(-c3cccc4oc5ccccc5c34)cc2)c2ccccc2-c2ccc3c4c(cccc24)-c2ccccc2O3)c1 ; c1cc(-c2ccc3c(c2)-c2cccc4cccc(c24)S3)cc(N(c2cccc(-c3cccc4ccccc34)c2)c2cccc3oc4ccccc4c23)c1 ; c1cc(-c2cccc3ccccc23)cc(N(c2ccc(-c3cccc4c3oc3ccccc34)cc2)c2cccc(-c3ccc4c5c(cccc35)-c3ccccc3O4)c2)c1]

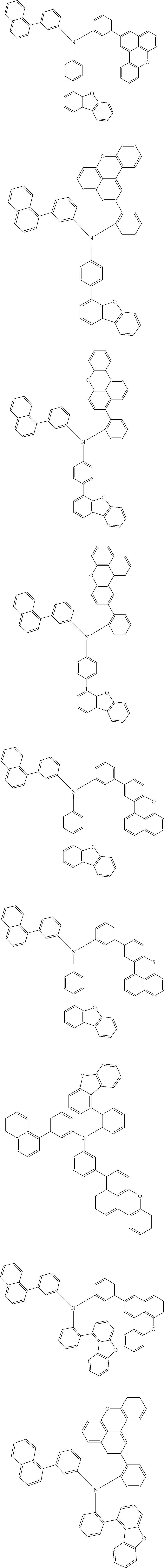

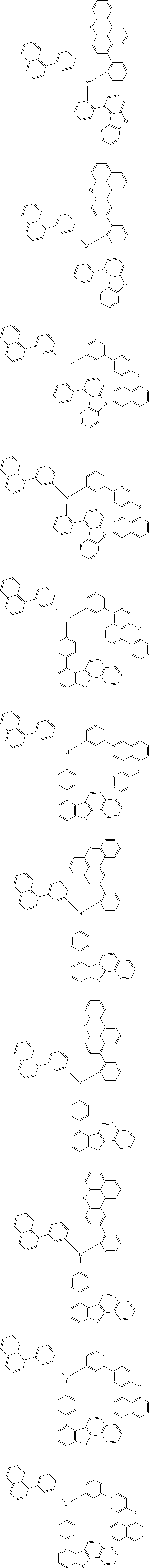

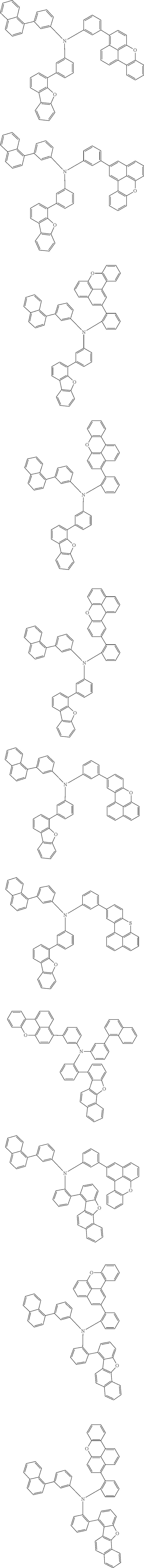

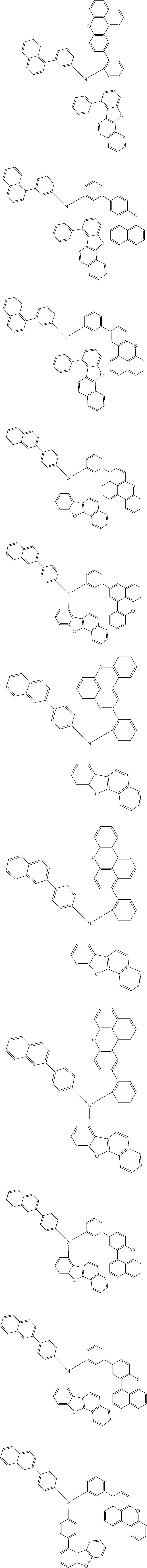

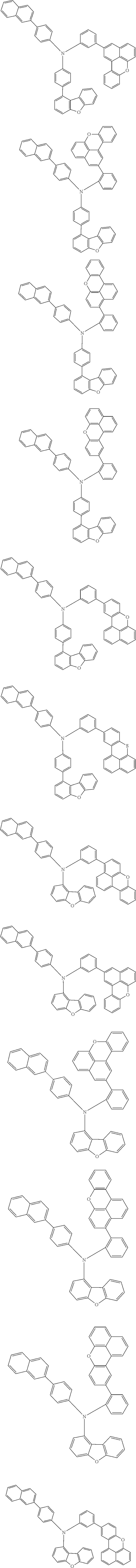

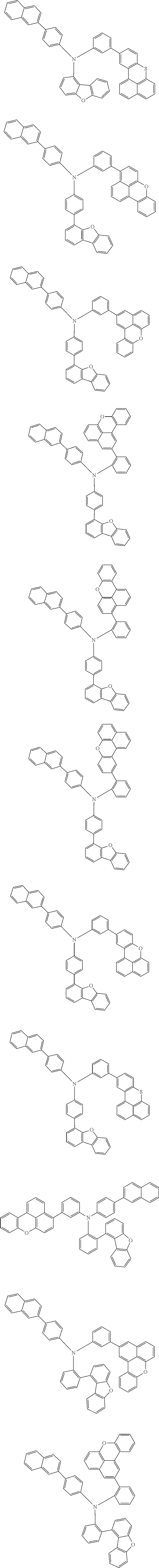

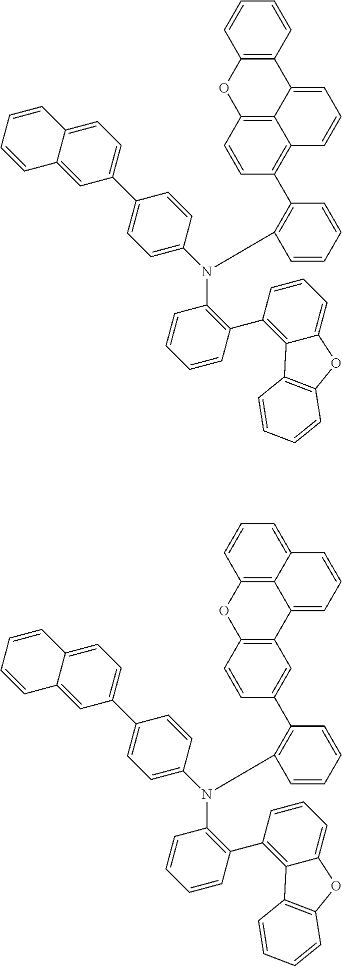 [SMILES: c1ccc2c(c1)Oc1ccc(-c3ccccc3N(c3ccc(-c4ccc5ccccc5c4)cc3)c3ccccc3-c3cccc4oc5ccccc5c34)c3cccc-2c13 ; c1ccc(N(c2ccc(-c3ccc4ccccc4c3)cc2)c2ccccc2-c2cccc3oc4ccccc4c23)c(-c2ccc3c(c2)-c2cccc4cccc(c24)O3)c1]

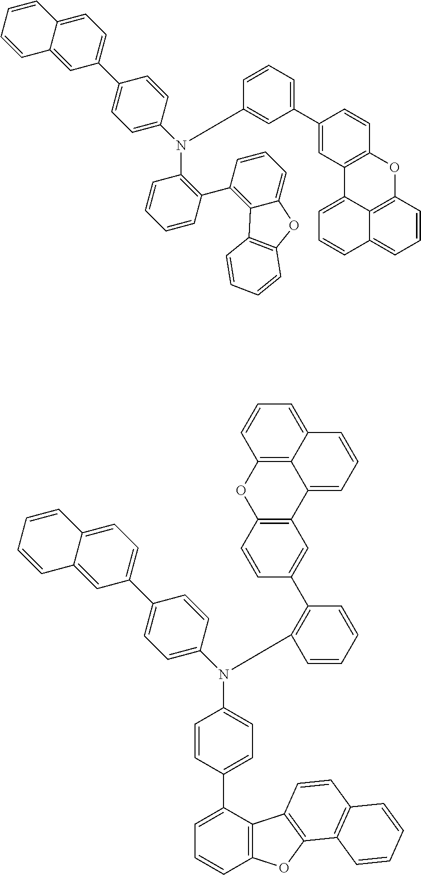 [SMILES: c1cc(-c2ccc3c(c2)-c2cccc4cccc(c24)O3)cc(N(c2ccc(-c3ccc4ccccc4c3)cc2)c2ccccc2-c2cccc3oc4ccccc4c23)c1 ; c1ccc(N(c2ccc(-c3ccc4ccccc4c3)cc2)c2ccc(-c3cccc4oc5c6ccccc6ccc5c34)cc2)c(-c2ccc3c(c2)-c2cccc4cccc(c24)O3)c1]

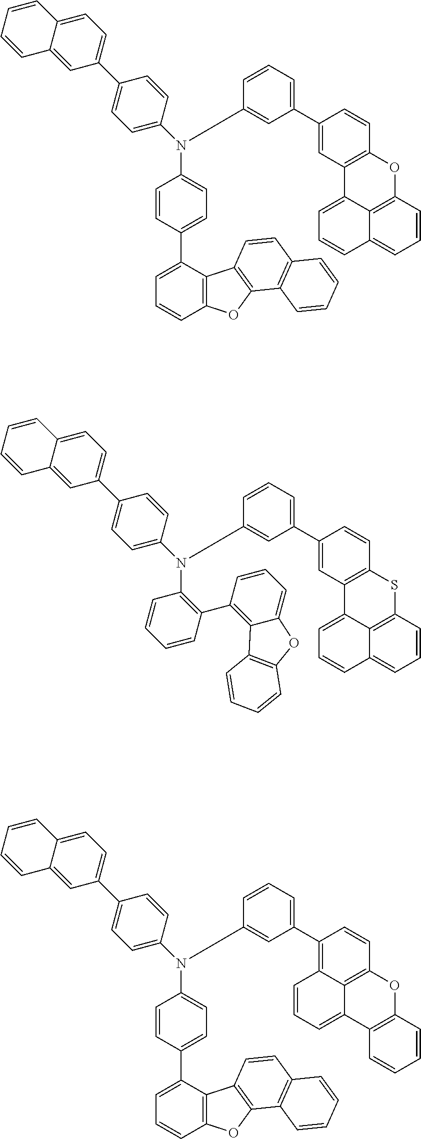 [SMILES: c1cc(-c2ccc3c(c2)-c2cccc4cccc(c24)O3)cc(N(c2ccc(-c3ccc4ccccc4c3)cc2)c2ccc(-c3cccc4oc5c6ccccc6ccc5c34)cc2)c1 ; c1cc(-c2ccc3c(c2)-c2cccc4cccc(c24)S3)cc(N(c2ccc(-c3ccc4ccccc4c3)cc2)c2ccccc2-c2cccc3oc4ccccc4c23)c1 ; c1cc(-c2ccc3c4c(cccc24)-c2ccccc2O3)cc(N(c2ccc(-c3ccc4ccccc4c3)cc2)c2ccc(-c3cccc4oc5c6ccccc6ccc5c34)cc2)c1]

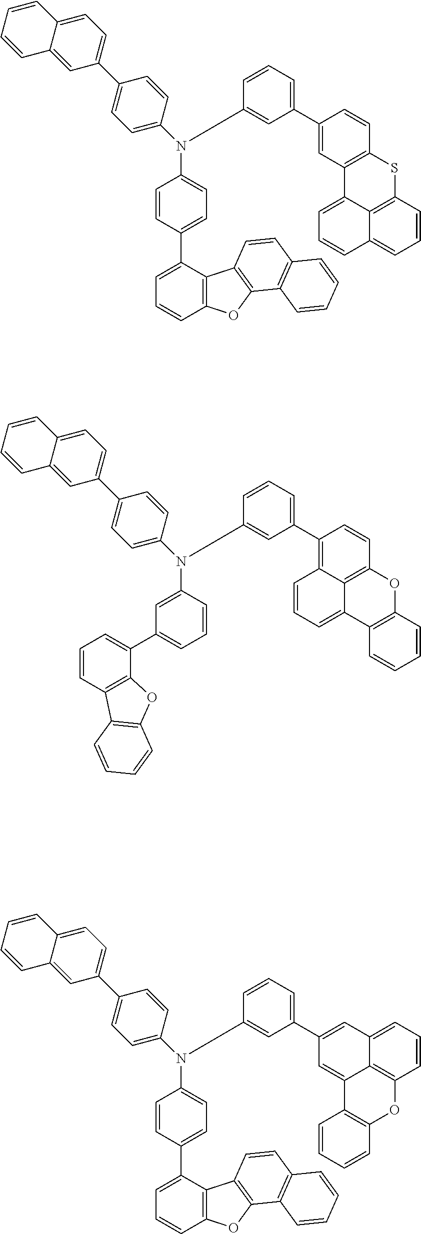 [SMILES: c1cc(-c2ccc3c(c2)-c2cccc4cccc(c24)S3)cc(N(c2ccc(-c3ccc4ccccc4c3)cc2)c2ccc(-c3cccc4oc5c6ccccc6ccc5c34)cc2)c1 ; c1cc(-c2ccc3c4c(cccc24)-c2ccccc2O3)cc(N(c2ccc(-c3ccc4ccccc4c3)cc2)c2cccc(-c3cccc4c3oc3ccccc34)c2)c1 ; c1cc(-c2cc3c4c(cccc4c2)Oc2ccccc2-3)cc(N(c2ccc(-c3ccc4ccccc4c3)cc2)c2ccc(-c3cccc4oc5c6ccccc6ccc5c34)cc2)c1]

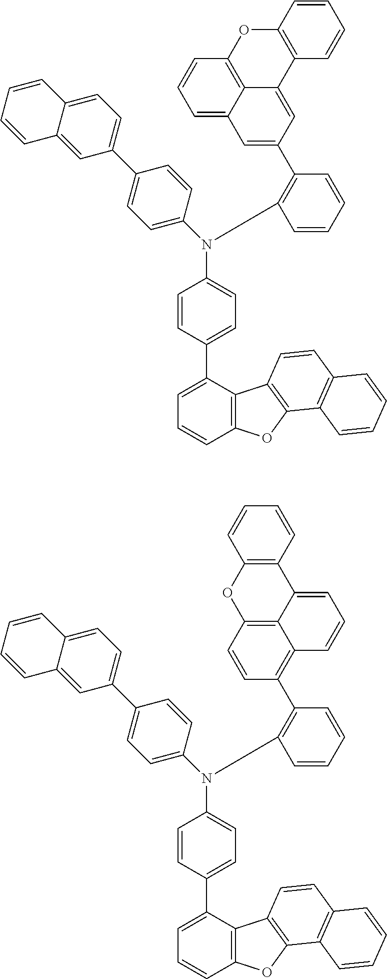 [SMILES: c1ccc2c(c1)Oc1cccc3cc(-c4ccccc4N(c4ccc(-c5ccc6ccccc6c5)cc4)c4ccc(-c5cccc6oc7c8ccccc8ccc7c56)cc4)cc-2c13 ; c1ccc2c(c1)Oc1ccc(-c3ccccc3N(c3ccc(-c4ccc5ccccc5c4)cc3)c3ccc(-c4cccc5oc6c7ccccc7ccc6c45)cc3)c3cccc-2c13]

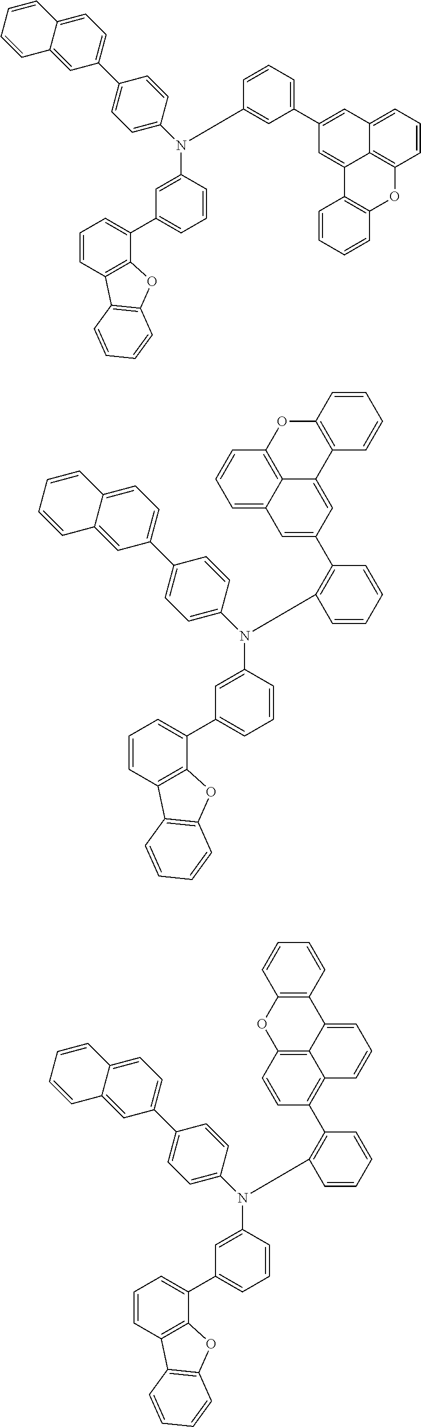 [SMILES: c1cc(-c2cc3c4c(cccc4c2)Oc2ccccc2-3)cc(N(c2ccc(-c3ccc4ccccc4c3)cc2)c2cccc(-c3cccc4c3oc3ccccc34)c2)c1 ; c1cc(-c2cccc3c2oc2ccccc23)cc(N(c2ccc(-c3ccc4ccccc4c3)cc2)c2ccccc2-c2cc3c4c(cccc4c2)Oc2ccccc2-3)c1 ; c1cc(-c2cccc3c2oc2ccccc23)cc(N(c2ccc(-c3ccc4ccccc4c3)cc2)c2ccccc2-c2ccc3c4c(cccc24)-c2ccccc2O3)c1]

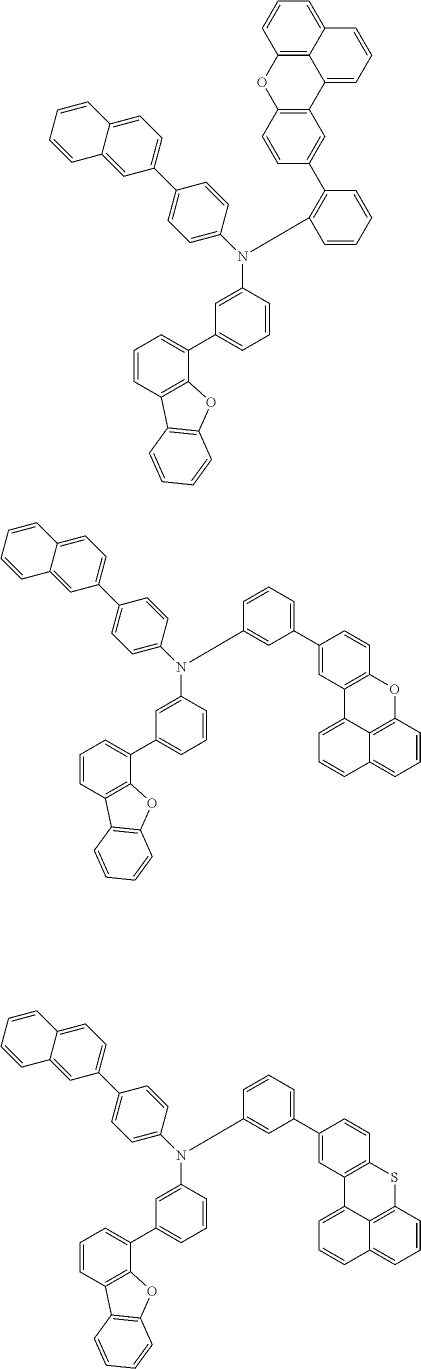 [SMILES: c1cc(-c2cccc3c2oc2ccccc23)cc(N(c2ccc(-c3ccc4ccccc4c3)cc2)c2ccccc2-c2ccc3c(c2)-c2cccc4cccc(c24)O3)c1 ; c1cc(-c2ccc3c(c2)-c2cccc4cccc(c24)O3)cc(N(c2ccc(-c3ccc4ccccc4c3)cc2)c2cccc(-c3cccc4c3oc3ccccc34)c2)c1 ; c1cc(-c2ccc3c(c2)-c2cccc4cccc(c24)S3)cc(N(c2ccc(-c3ccc4ccccc4c3)cc2)c2cccc(-c3cccc4c3oc3ccccc34)c2)c1]

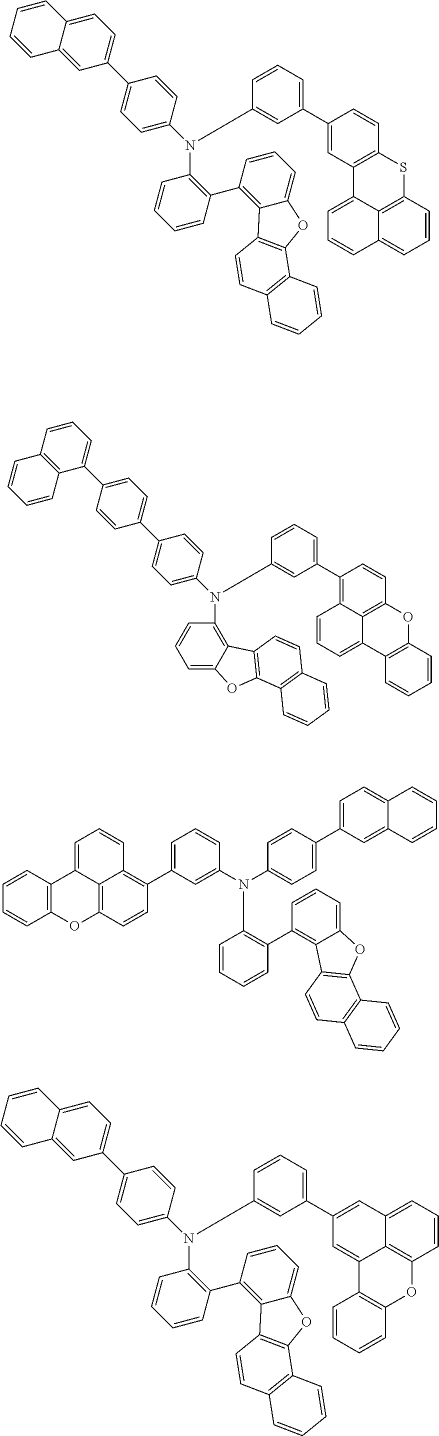 [SMILES: c1cc(-c2ccc3c(c2)-c2cccc4cccc(c24)S3)cc(N(c2ccc(-c3ccc4ccccc4c3)cc2)c2ccccc2-c2cccc3oc4c5ccccc5ccc4c23)c1 ; c1cc(-c2ccc3c4c(cccc24)-c2ccccc2O3)cc(N(c2ccc(-c3ccc(-c4cccc5ccccc45)cc3)cc2)c2cccc3oc4c5ccccc5ccc4c23)c1 ; c1cc(-c2ccc3c4c(cccc24)-c2ccccc2O3)cc(N(c2ccc(-c3ccc4ccccc4c3)cc2)c2ccccc2-c2cccc3oc4c5ccccc5ccc4c23)c1 ; c1cc(-c2cc3c4c(cccc4c2)Oc2ccccc2-3)cc(N(c2ccc(-c3ccc4ccccc4c3)cc2)c2ccccc2-c2cccc3oc4c5ccccc5ccc4c23)c1]

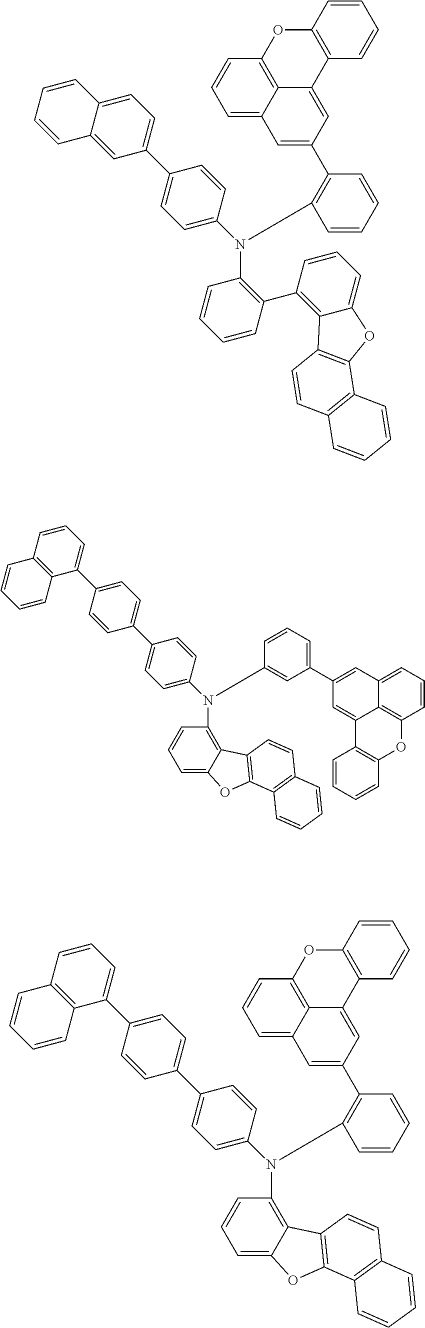 [SMILES: c1ccc2c(c1)Oc1cccc3cc(-c4ccccc4N(c4ccc(-c5ccc6ccccc6c5)cc4)c4ccccc4-c4cccc5oc6c7ccccc7ccc6c45)cc-2c13 ; c1cc(-c2cc3c4c(cccc4c2)Oc2ccccc2-3)cc(N(c2ccc(-c3ccc(-c4cccc5ccccc45)cc3)cc2)c2cccc3oc4c5ccccc5ccc4c23)c1 ; c1ccc2c(c1)Oc1cccc3cc(-c4ccccc4N(c4ccc(-c5ccc(-c6cccc7ccccc67)cc5)cc4)c4cccc5oc6c7ccccc7ccc6c45)cc-2c13]

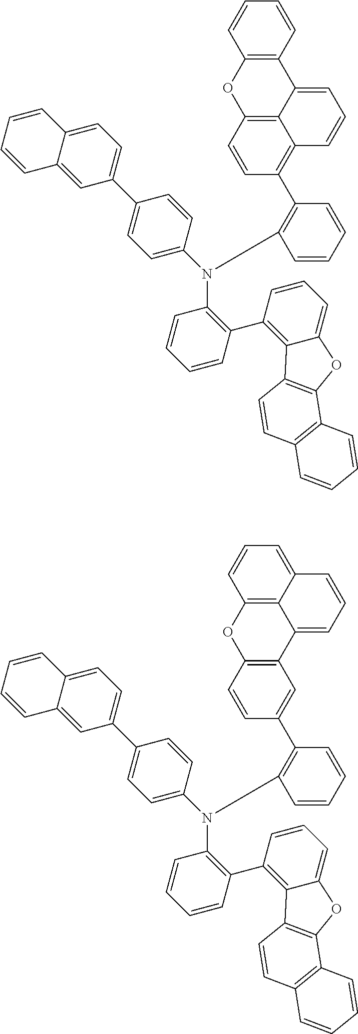 [SMILES: c1ccc2c(c1)Oc1ccc(-c3ccccc3N(c3ccc(-c4ccc5ccccc5c4)cc3)c3ccccc3-c3cccc4oc5c6ccccc6ccc5c34)c3cccc-2c13 ; c1ccc(N(c2ccc(-c3ccc4ccccc4c3)cc2)c2ccccc2-c2cccc3oc4c5ccccc5ccc4c23)c(-c2ccc3c(c2)-c2cccc4cccc(c24)O3)c1]

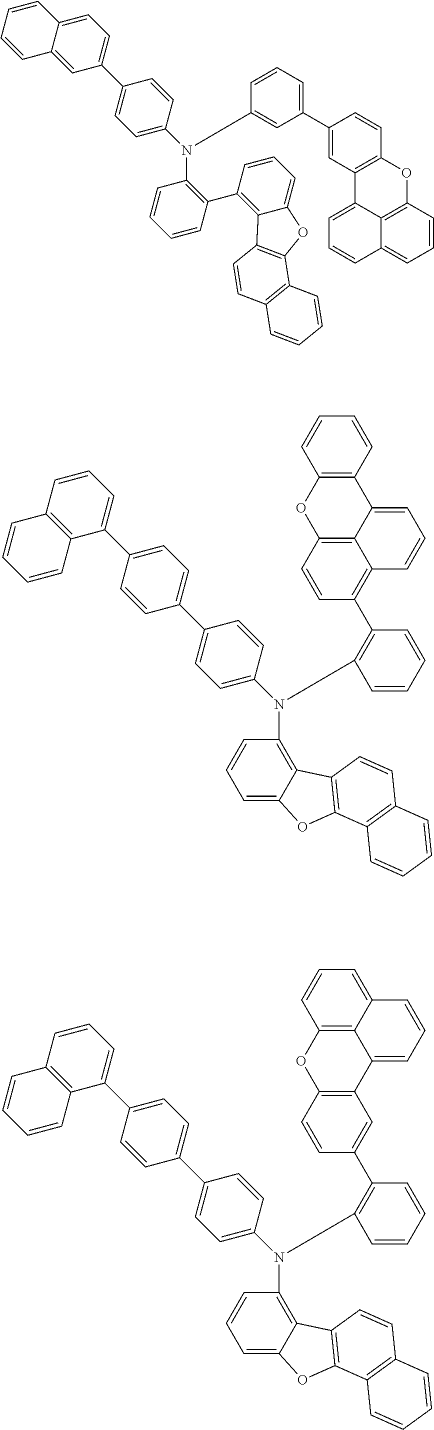 [SMILES: c1cc(-c2ccc3c(c2)-c2cccc4cccc(c24)O3)cc(N(c2ccc(-c3ccc4ccccc4c3)cc2)c2ccccc2-c2cccc3oc4c5ccccc5ccc4c23)c1 ; c1ccc2c(c1)Oc1ccc(-c3ccccc3N(c3ccc(-c4ccc(-c5cccc6ccccc56)cc4)cc3)c3cccc4oc5c6ccccc6ccc5c34)c3cccc-2c13 ; c1ccc(N(c2ccc(-c3ccc(-c4cccc5ccccc45)cc3)cc2)c2cccc3oc4c5ccccc5ccc4c23)c(-c2ccc3c(c2)-c2cccc4cccc(c24)O3)c1]

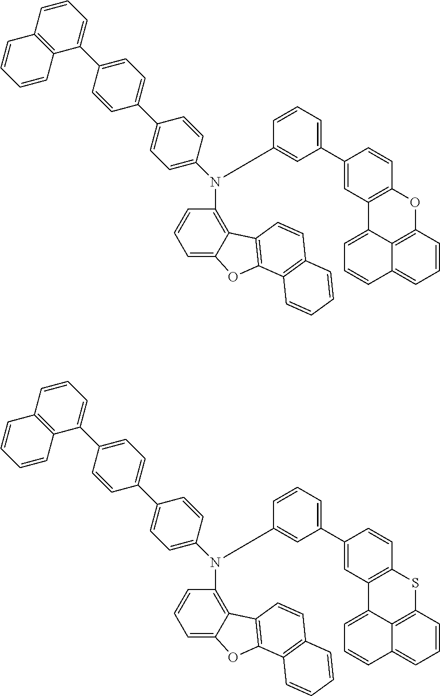 [SMILES: c1cc(-c2ccc3c(c2)-c2cccc4cccc(c24)O3)cc(N(c2ccc(-c3ccc(-c4cccc5ccccc45)cc3)cc2)c2cccc3oc4c5ccccc5ccc4c23)c1 ; c1cc(-c2ccc3c(c2)-c2cccc4cccc(c24)S3)cc(N(c2ccc(-c3ccc(-c4cccc5ccccc45)cc3)cc2)c2cccc3oc4c5ccccc5ccc4c23)c1]

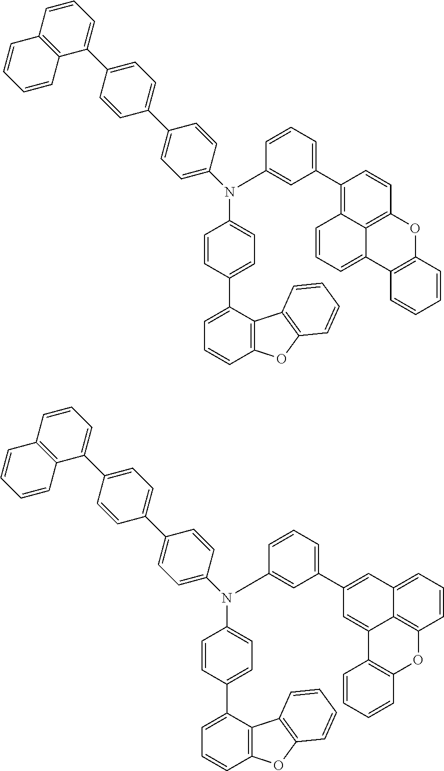 [SMILES: c1cc(-c2ccc3c4c(cccc24)-c2ccccc2O3)cc(N(c2ccc(-c3ccc(-c4cccc5ccccc45)cc3)cc2)c2ccc(-c3cccc4oc5ccccc5c34)cc2)c1 ; c1cc(-c2cc3c4c(cccc4c2)Oc2ccccc2-3)cc(N(c2ccc(-c3ccc(-c4cccc5ccccc45)cc3)cc2)c2ccc(-c3cccc4oc5ccccc5c34)cc2)c1]

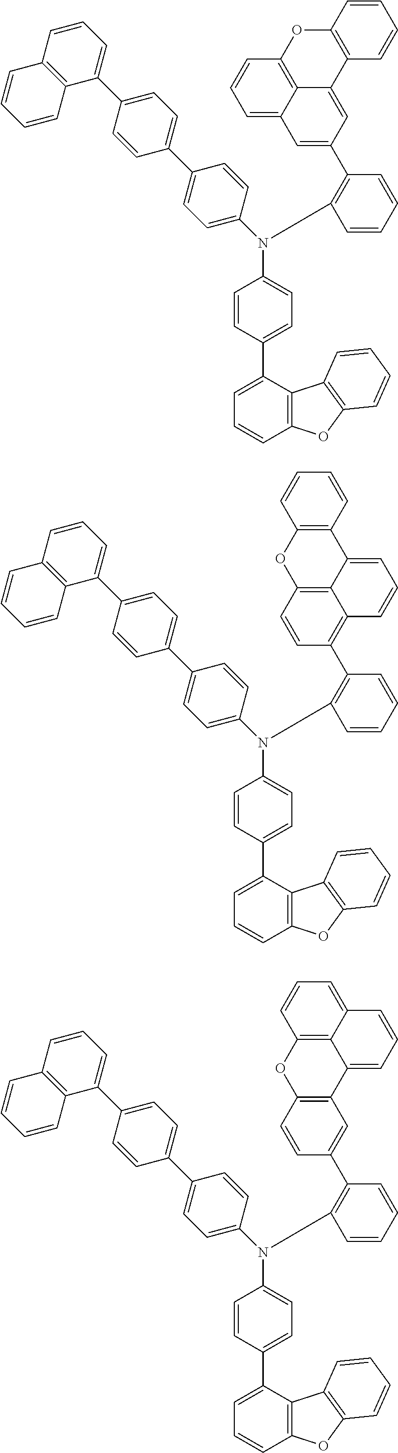

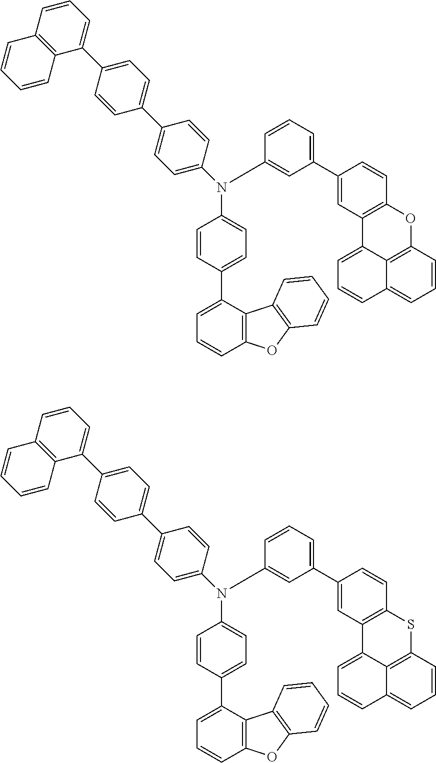 [SMILES: c1cc(-c2ccc3c(c2)-c2cccc4cccc(c24)O3)cc(N(c2ccc(-c3ccc(-c4cccc5ccccc45)cc3)cc2)c2ccc(-c3cccc4oc5ccccc5c34)cc2)c1 ; c1cc(-c2ccc3c(c2)-c2cccc4cccc(c24)S3)cc(N(c2ccc(-c3ccc(-c4cccc5ccccc45)cc3)cc2)c2ccc(-c3cccc4oc5ccccc5c34)cc2)c1]

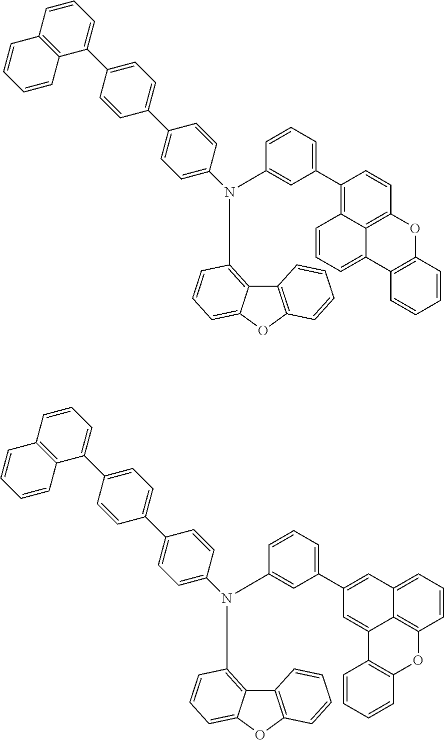 [SMILES: c1cc(-c2ccc3c4c(cccc24)-c2ccccc2O3)cc(N(c2ccc(-c3ccc(-c4cccc5ccccc45)cc3)cc2)c2cccc3oc4ccccc4c23)c1 ; c1cc(-c2cc3c4c(cccc4c2)Oc2ccccc2-3)cc(N(c2ccc(-c3ccc(-c4cccc5ccccc45)cc3)cc2)c2cccc3oc4ccccc4c23)c1]

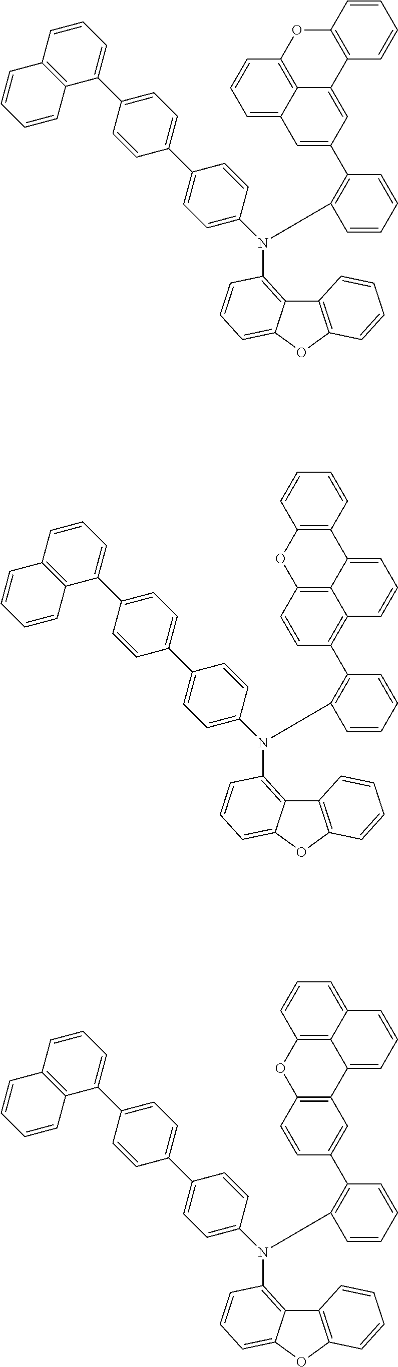 [SMILES: c1ccc2c(c1)Oc1cccc3cc(-c4ccccc4N(c4ccc(-c5ccc(-c6cccc7ccccc67)cc5)cc4)c4cccc5oc6ccccc6c45)cc-2c13 ; c1ccc2c(c1)Oc1ccc(-c3ccccc3N(c3ccc(-c4ccc(-c5cccc6ccccc56)cc4)cc3)c3cccc4oc5ccccc5c34)c3cccc-2c13 ; c1ccc(N(c2ccc(-c3ccc(-c4cccc5ccccc45)cc3)cc2)c2cccc3oc4ccccc4c23)c(-c2ccc3c(c2)-c2cccc4cccc(c24)O3)c1]

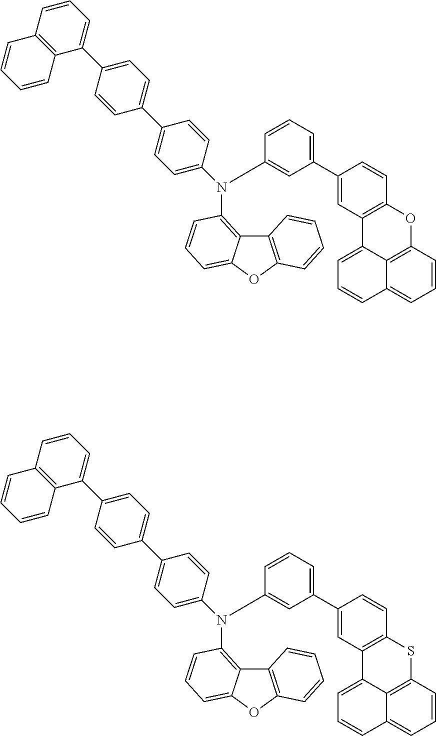 [SMILES: c1cc(-c2ccc3c(c2)-c2cccc4cccc(c24)O3)cc(N(c2ccc(-c3ccc(-c4cccc5ccccc45)cc3)cc2)c2cccc3oc4ccccc4c23)c1 ; c1cc(-c2ccc3c(c2)-c2cccc4cccc(c24)S3)cc(N(c2ccc(-c3ccc(-c4cccc5ccccc45)cc3)cc2)c2cccc3oc4ccccc4c23)c1]

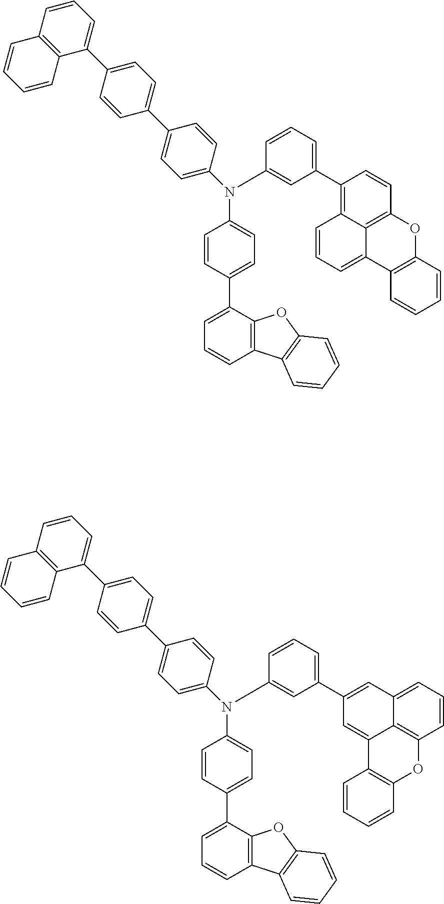 [SMILES: c1cc(-c2ccc3c4c(cccc24)-c2ccccc2O3)cc(N(c2ccc(-c3ccc(-c4cccc5ccccc45)cc3)cc2)c2ccc(-c3cccc4c3oc3ccccc34)cc2)c1 ; c1cc(-c2cc3c4c(cccc4c2)Oc2ccccc2-3)cc(N(c2ccc(-c3ccc(-c4cccc5ccccc45)cc3)cc2)c2ccc(-c3cccc4c3oc3ccccc34)cc2)c1]

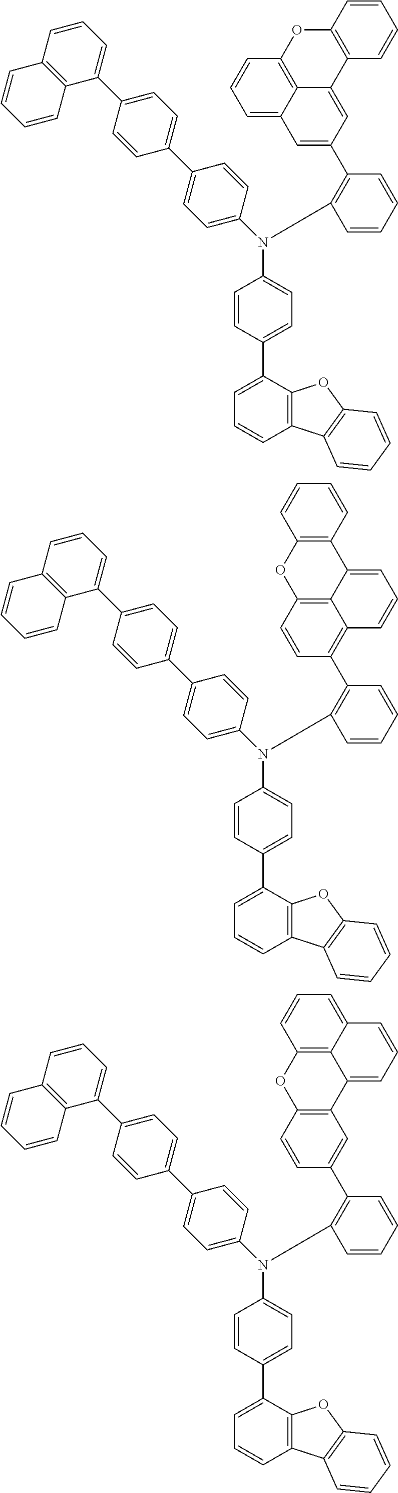

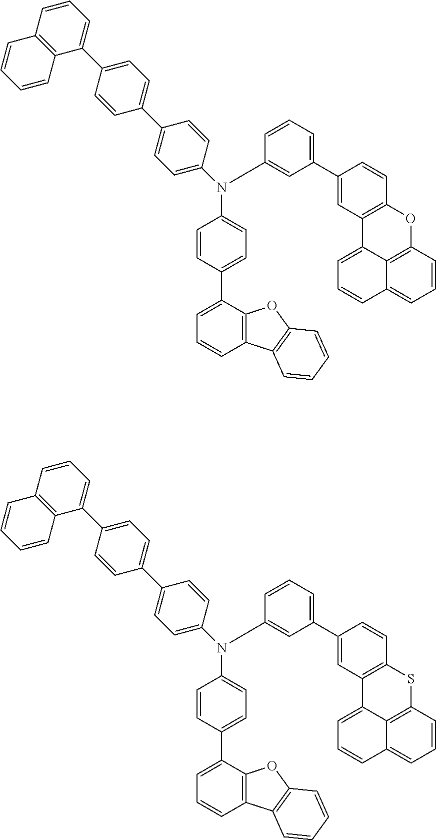 [SMILES: c1cc(-c2ccc3c(c2)-c2cccc4cccc(c24)O3)cc(N(c2ccc(-c3ccc(-c4cccc5ccccc45)cc3)cc2)c2ccc(-c3cccc4c3oc3ccccc34)cc2)c1 ; c1cc(-c2ccc3c(c2)-c2cccc4cccc(c24)S3)cc(N(c2ccc(-c3ccc(-c4cccc5ccccc45)cc3)cc2)c2ccc(-c3cccc4c3oc3ccccc34)cc2)c1]

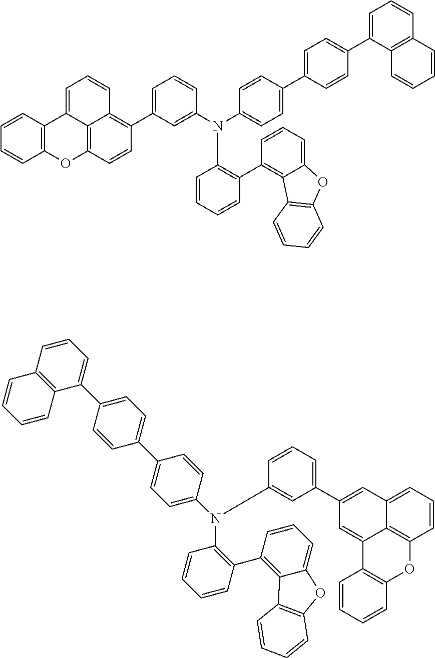 [SMILES: c1cc(-c2ccc3c4c(cccc24)-c2ccccc2O3)cc(N(c2ccc(-c3ccc(-c4cccc5ccccc45)cc3)cc2)c2ccccc2-c2cccc3oc4ccccc4c23)c1 ; c1cc(-c2cc3c4c(cccc4c2)Oc2ccccc2-3)cc(N(c2ccc(-c3ccc(-c4cccc5ccccc45)cc3)cc2)c2ccccc2-c2cccc3oc4ccccc4c23)c1]

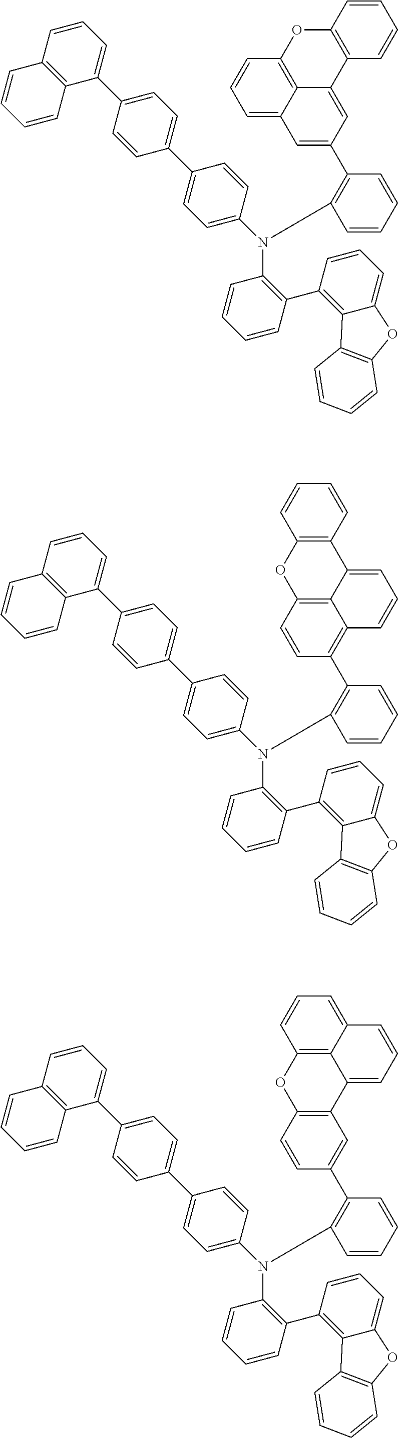 [SMILES: c1ccc2c(c1)Oc1cccc3cc(-c4ccccc4N(c4ccc(-c5ccc(-c6cccc7ccccc67)cc5)cc4)c4ccccc4-c4cccc5oc6ccccc6c45)cc-2c13 ; c1ccc2c(c1)Oc1ccc(-c3ccccc3N(c3ccc(-c4ccc(-c5cccc6ccccc56)cc4)cc3)c3ccccc3-c3cccc4oc5ccccc5c34)c3cccc-2c13 ; c1ccc(N(c2ccc(-c3ccc(-c4cccc5ccccc45)cc3)cc2)c2ccccc2-c2cccc3oc4ccccc4c23)c(-c2ccc3c(c2)-c2cccc4cccc(c24)O3)c1]

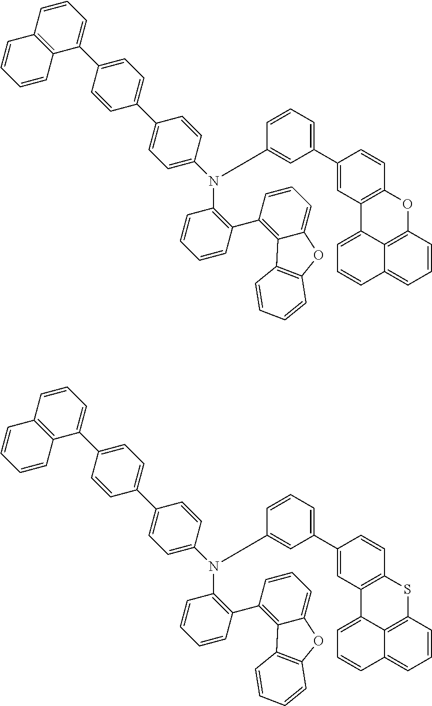 [SMILES: c1cc(-c2ccc3c(c2)-c2cccc4cccc(c24)O3)cc(N(c2ccc(-c3ccc(-c4cccc5ccccc45)cc3)cc2)c2ccccc2-c2cccc3oc4ccccc4c23)c1 ; c1cc(-c2ccc3c(c2)-c2cccc4cccc(c24)S3)cc(N(c2ccc(-c3ccc(-c4cccc5ccccc45)cc3)cc2)c2ccccc2-c2cccc3oc4ccccc4c23)c1]

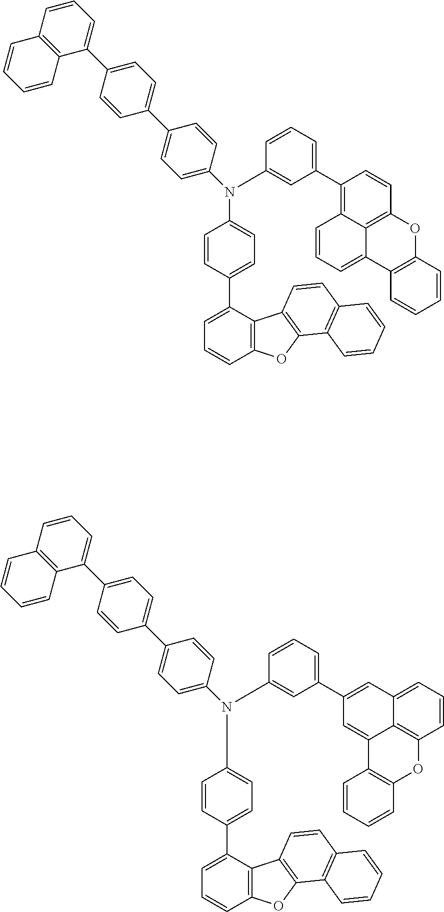 [SMILES: c1cc(-c2ccc3c4c(cccc24)-c2ccccc2O3)cc(N(c2ccc(-c3ccc(-c4cccc5ccccc45)cc3)cc2)c2ccc(-c3cccc4oc5c6ccccc6ccc5c34)cc2)c1 ; c1cc(-c2cc3c4c(cccc4c2)Oc2ccccc2-3)cc(N(c2ccc(-c3ccc(-c4cccc5ccccc45)cc3)cc2)c2ccc(-c3cccc4oc5c6ccccc6ccc5c34)cc2)c1]

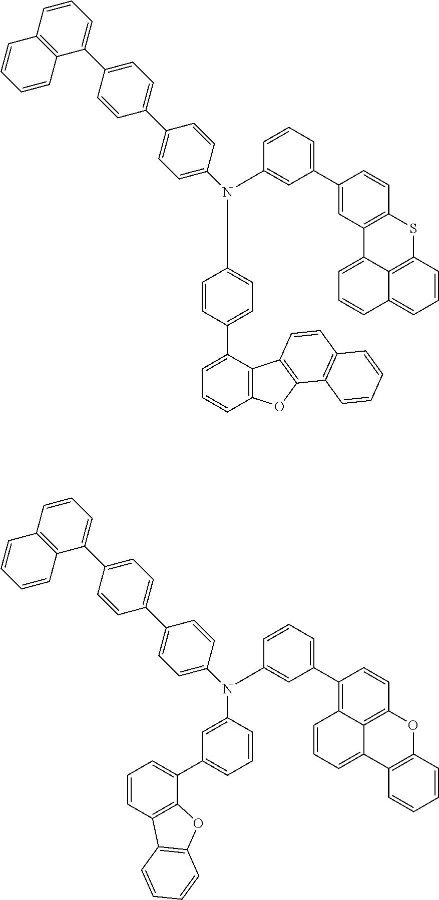 [SMILES: c1cc(-c2ccc3c(c2)-c2cccc4cccc(c24)S3)cc(N(c2ccc(-c3ccc(-c4cccc5ccccc45)cc3)cc2)c2ccc(-c3cccc4oc5c6ccccc6ccc5c34)cc2)c1 ; c1cc(-c2ccc3c4c(cccc24)-c2ccccc2O3)cc(N(c2ccc(-c3ccc(-c4cccc5ccccc45)cc3)cc2)c2cccc(-c3cccc4c3oc3ccccc34)c2)c1]

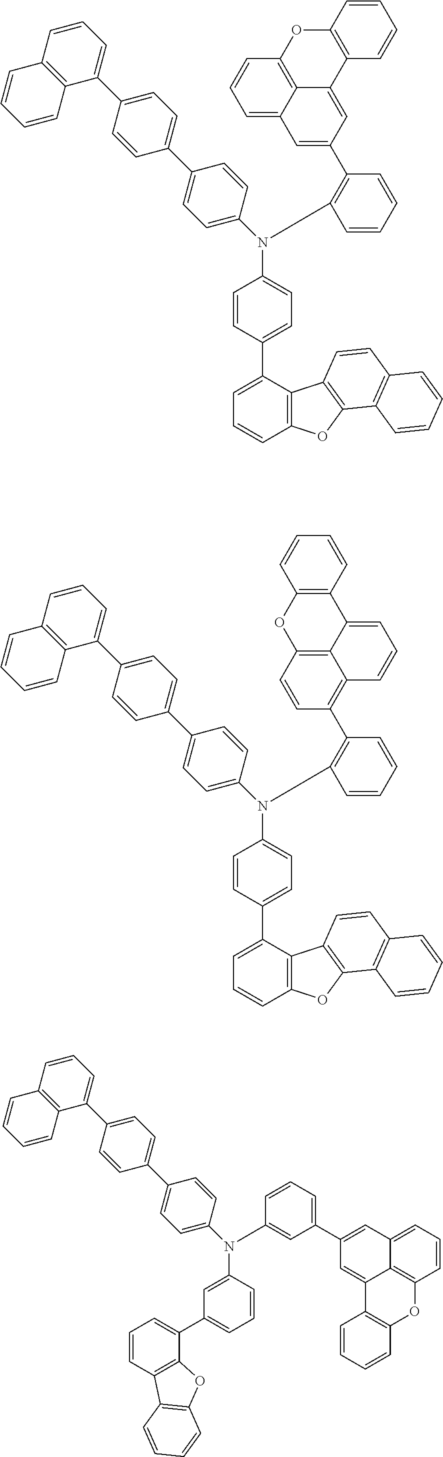 [SMILES: c1ccc2c(c1)Oc1cccc3cc(-c4ccccc4N(c4ccc(-c5ccc(-c6cccc7ccccc67)cc5)cc4)c4ccc(-c5cccc6oc7c8ccccc8ccc7c56)cc4)cc-2c13 ; c1ccc2c(c1)Oc1ccc(-c3ccccc3N(c3ccc(-c4ccc(-c5cccc6ccccc56)cc4)cc3)c3ccc(-c4cccc5oc6c7ccccc7ccc6c45)cc3)c3cccc-2c13 ; c1cc(-c2cc3c4c(cccc4c2)Oc2ccccc2-3)cc(N(c2ccc(-c3ccc(-c4cccc5ccccc45)cc3)cc2)c2cccc(-c3cccc4c3oc3ccccc34)c2)c1]

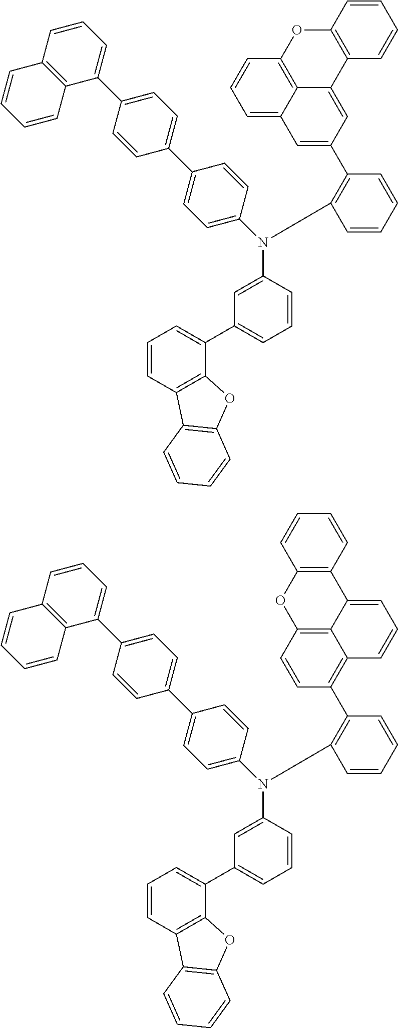 [SMILES: c1cc(-c2cccc3c2oc2ccccc23)cc(N(c2ccc(-c3ccc(-c4cccc5ccccc45)cc3)cc2)c2ccccc2-c2cc3c4c(cccc4c2)Oc2ccccc2-3)c1 ; c1cc(-c2cccc3c2oc2ccccc23)cc(N(c2ccc(-c3ccc(-c4cccc5ccccc45)cc3)cc2)c2ccccc2-c2ccc3c4c(cccc24)-c2ccccc2O3)c1]

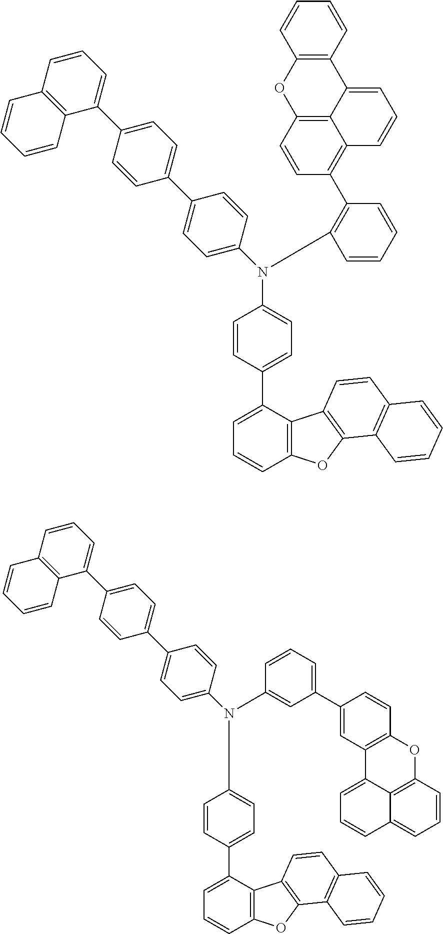 [SMILES: c1ccc2c(c1)Oc1ccc(-c3ccccc3N(c3ccc(-c4ccc(-c5cccc6ccccc56)cc4)cc3)c3ccc(-c4cccc5oc6c7ccccc7ccc6c45)cc3)c3cccc-2c13 ; c1cc(-c2ccc3c(c2)-c2cccc4cccc(c24)O3)cc(N(c2ccc(-c3ccc(-c4cccc5ccccc45)cc3)cc2)c2ccc(-c3cccc4oc5c6ccccc6ccc5c34)cc2)c1]

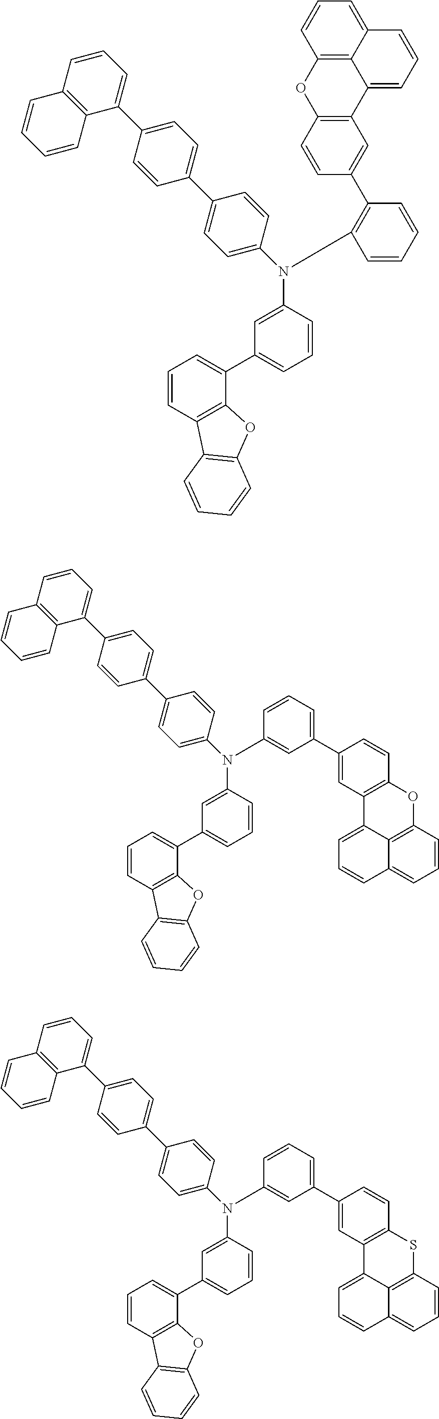 [SMILES: c1cc(-c2cccc3c2oc2ccccc23)cc(N(c2ccc(-c3ccc(-c4cccc5ccccc45)cc3)cc2)c2ccccc2-c2ccc3c(c2)-c2cccc4cccc(c24)O3)c1 ; c1cc(-c2ccc3c(c2)-c2cccc4cccc(c24)O3)cc(N(c2ccc(-c3ccc(-c4cccc5ccccc45)cc3)cc2)c2cccc(-c3cccc4c3oc3ccccc34)c2)c1 ; c1cc(-c2ccc3c(c2)-c2cccc4cccc(c24)S3)cc(N(c2ccc(-c3ccc(-c4cccc5ccccc45)cc3)cc2)c2cccc(-c3cccc4c3oc3ccccc34)c2)c1]

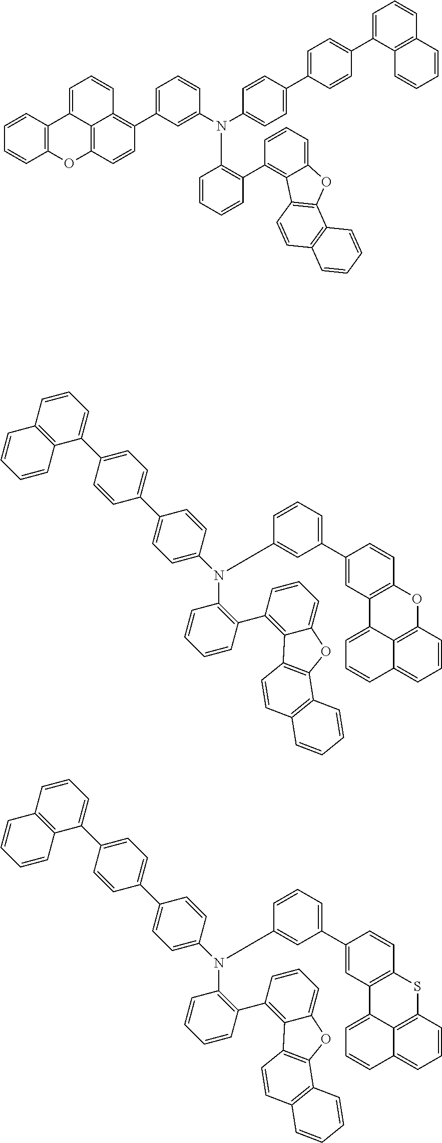 [SMILES: c1cc(-c2ccc3c4c(cccc24)-c2ccccc2O3)cc(N(c2ccc(-c3ccc(-c4cccc5ccccc45)cc3)cc2)c2ccccc2-c2cccc3oc4c5ccccc5ccc4c23)c1 ; c1cc(-c2ccc3c(c2)-c2cccc4cccc(c24)O3)cc(N(c2ccc(-c3ccc(-c4cccc5ccccc45)cc3)cc2)c2ccccc2-c2cccc3oc4c5ccccc5ccc4c23)c1 ; c1cc(-c2ccc3c(c2)-c2cccc4cccc(c24)S3)cc(N(c2ccc(-c3ccc(-c4cccc5ccccc45)cc3)cc2)c2ccccc2-c2cccc3oc4c5ccccc5ccc4c23)c1]

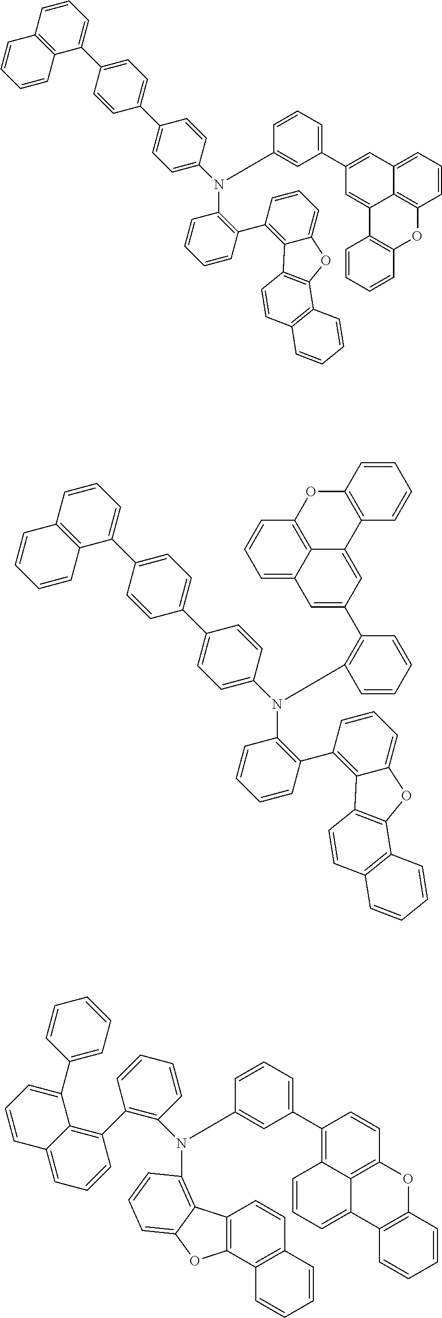 [SMILES: c1cc(-c2cc3c4c(cccc4c2)Oc2ccccc2-3)cc(N(c2ccc(-c3ccc(-c4cccc5ccccc45)cc3)cc2)c2ccccc2-c2cccc3oc4c5ccccc5ccc4c23)c1 ; c1ccc2c(c1)Oc1cccc3cc(-c4ccccc4N(c4ccc(-c5ccc(-c6cccc7ccccc67)cc5)cc4)c4ccccc4-c4cccc5oc6c7ccccc7ccc6c45)cc-2c13 ; c1ccc(-c2cccc3cccc(-c4ccccc4N(c4cccc(-c5ccc6c7c(cccc57)-c5ccccc5O6)c4)c4cccc5oc6c7ccccc7ccc6c45)c23)cc1]

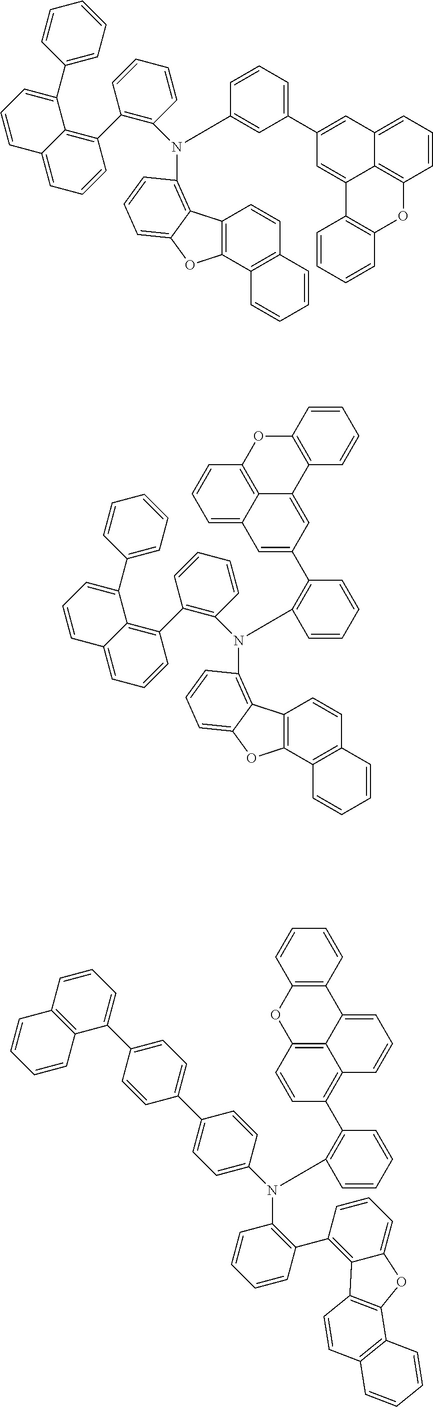 [SMILES: c1ccc(-c2cccc3cccc(-c4ccccc4N(c4cccc(-c5cc6c7c(cccc7c5)Oc5ccccc5-6)c4)c4cccc5oc6c7ccccc7ccc6c45)c23)cc1 ; c1ccc(-c2cccc3cccc(-c4ccccc4N(c4ccccc4-c4cc5c6c(cccc6c4)Oc4ccccc4-5)c4cccc5oc6c7ccccc7ccc6c45)c23)cc1 ; c1ccc2c(c1)Oc1ccc(-c3ccccc3N(c3ccc(-c4ccc(-c5cccc6ccccc56)cc4)cc3)c3ccccc3-c3cccc4oc5c6ccccc6ccc5c34)c3cccc-2c13]

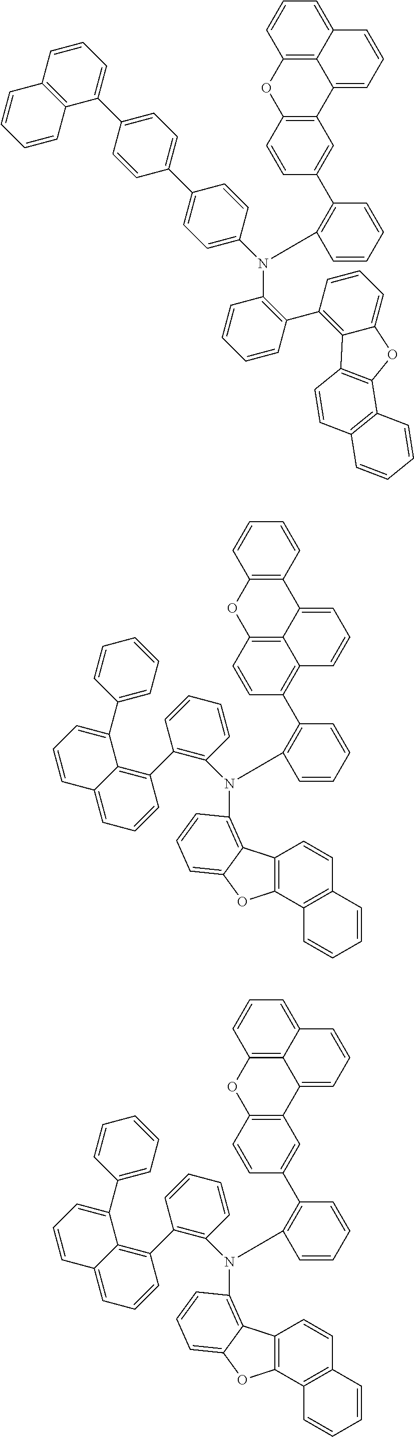 [SMILES: c1ccc(N(c2ccc(-c3ccc(-c4cccc5ccccc45)cc3)cc2)c2ccccc2-c2cccc3oc4c5ccccc5ccc4c23)c(-c2ccc3c(c2)-c2cccc4cccc(c24)O3)c1 ; c1ccc(-c2cccc3cccc(-c4ccccc4N(c4ccccc4-c4ccc5c6c(cccc46)-c4ccccc4O5)c4cccc5oc6c7ccccc7ccc6c45)c23)cc1 ; c1ccc(-c2cccc3cccc(-c4ccccc4N(c4ccccc4-c4ccc5c(c4)-c4cccc6cccc(c46)O5)c4cccc5oc6c7ccccc7ccc6c45)c23)cc1]

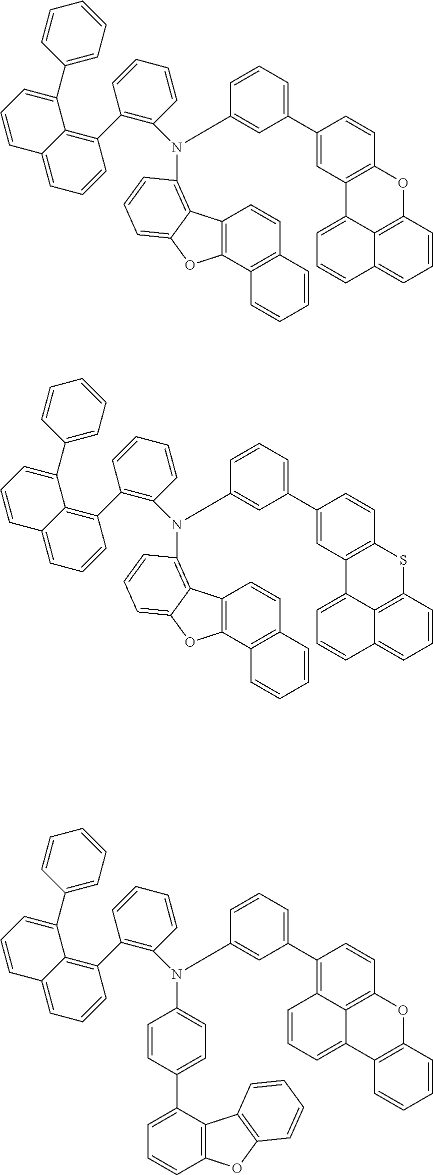 [SMILES: c1ccc(-c2cccc3cccc(-c4ccccc4N(c4cccc(-c5ccc6c(c5)-c5cccc7cccc(c57)O6)c4)c4cccc5oc6c7ccccc7ccc6c45)c23)cc1 ; c1ccc(-c2cccc3cccc(-c4ccccc4N(c4cccc(-c5ccc6c(c5)-c5cccc7cccc(c57)S6)c4)c4cccc5oc6c7ccccc7ccc6c45)c23)cc1 ; c1ccc(-c2cccc3cccc(-c4ccccc4N(c4ccc(-c5cccc6oc7ccccc7c56)cc4)c4cccc(-c5ccc6c7c(cccc57)-c5ccccc5O6)c4)c23)cc1]

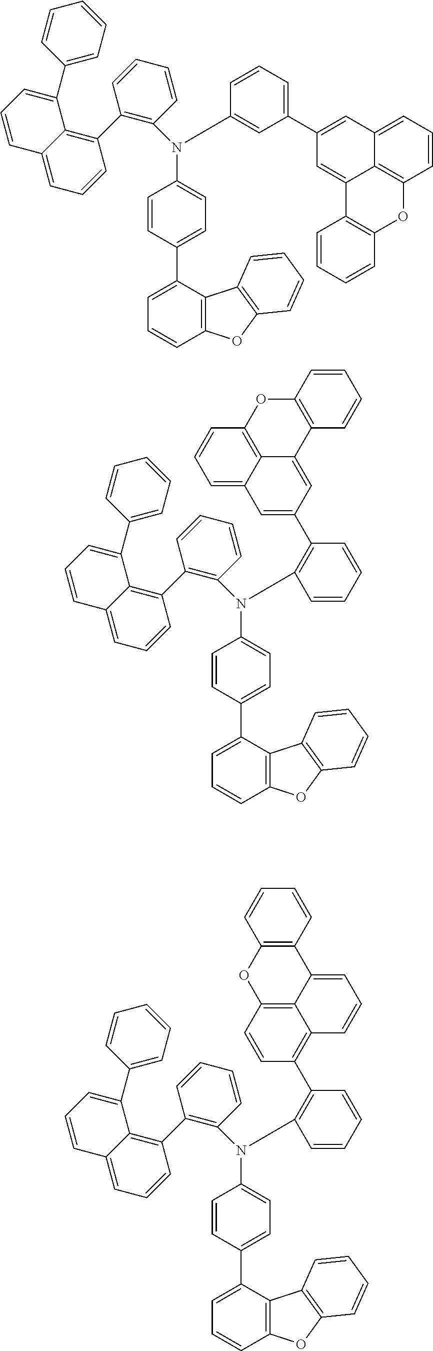 [SMILES: c1ccc(-c2cccc3cccc(-c4ccccc4N(c4ccc(-c5cccc6oc7ccccc7c56)cc4)c4cccc(-c5cc6c7c(cccc7c5)Oc5ccccc5-6)c4)c23)cc1 ; c1ccc(-c2cccc3cccc(-c4ccccc4N(c4ccc(-c5cccc6oc7ccccc7c56)cc4)c4ccccc4-c4cc5c6c(cccc6c4)Oc4ccccc4-5)c23)cc1 ; c1ccc(-c2cccc3cccc(-c4ccccc4N(c4ccc(-c5cccc6oc7ccccc7c56)cc4)c4ccccc4-c4ccc5c6c(cccc46)-c4ccccc4O5)c23)cc1]

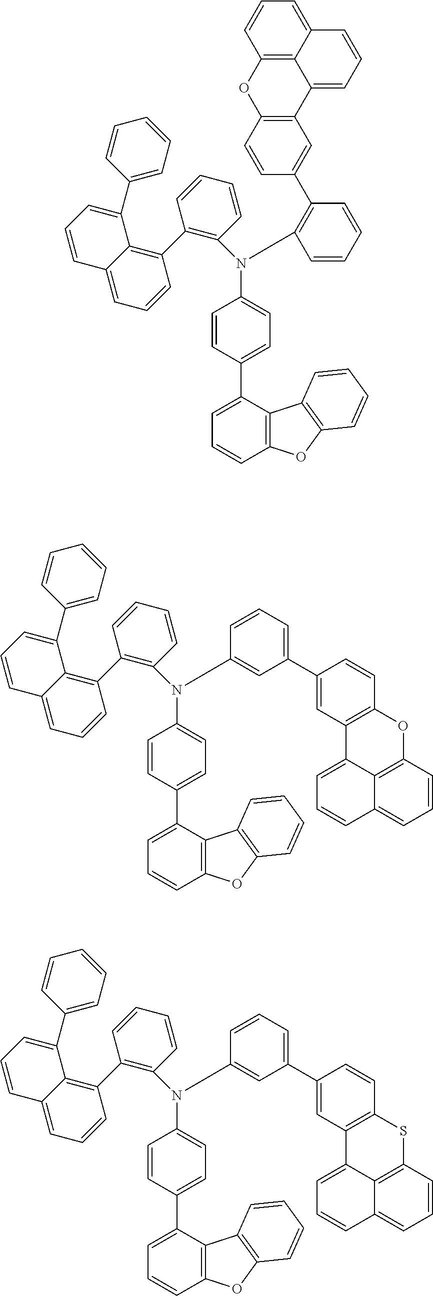 [SMILES: c1ccc(-c2cccc3cccc(-c4ccccc4N(c4ccc(-c5cccc6oc7ccccc7c56)cc4)c4ccccc4-c4ccc5c(c4)-c4cccc6cccc(c46)O5)c23)cc1 ; c1ccc(-c2cccc3cccc(-c4ccccc4N(c4ccc(-c5cccc6oc7ccccc7c56)cc4)c4cccc(-c5ccc6c(c5)-c5cccc7cccc(c57)O6)c4)c23)cc1 ; c1ccc(-c2cccc3cccc(-c4ccccc4N(c4ccc(-c5cccc6oc7ccccc7c56)cc4)c4cccc(-c5ccc6c(c5)-c5cccc7cccc(c57)S6)c4)c23)cc1]

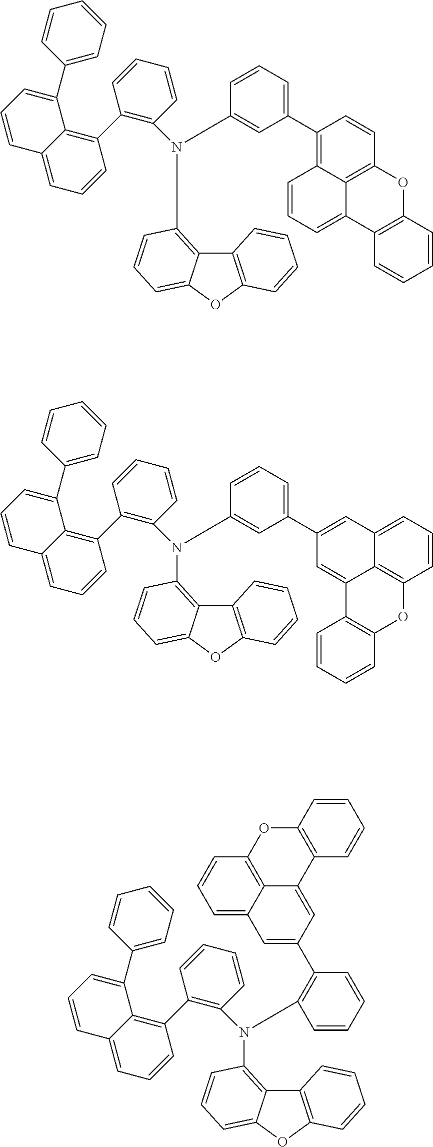 [SMILES: c1ccc(-c2cccc3cccc(-c4ccccc4N(c4cccc(-c5ccc6c7c(cccc57)-c5ccccc5O6)c4)c4cccc5oc6ccccc6c45)c23)cc1 ; c1ccc(-c2cccc3cccc(-c4ccccc4N(c4cccc(-c5cc6c7c(cccc7c5)Oc5ccccc5-6)c4)c4cccc5oc6ccccc6c45)c23)cc1 ; c1ccc(-c2cccc3cccc(-c4ccccc4N(c4ccccc4-c4cc5c6c(cccc6c4)Oc4ccccc4-5)c4cccc5oc6ccccc6c45)c23)cc1]

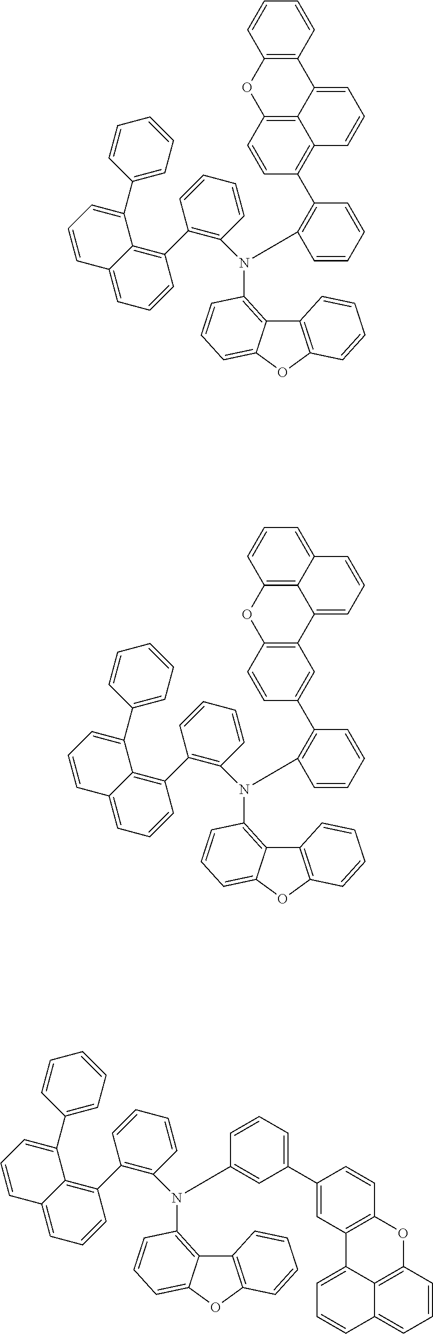 [SMILES: c1ccc(-c2cccc3cccc(-c4ccccc4N(c4ccccc4-c4ccc5c6c(cccc46)-c4ccccc4O5)c4cccc5oc6ccccc6c45)c23)cc1 ; c1ccc(-c2cccc3cccc(-c4ccccc4N(c4ccccc4-c4ccc5c(c4)-c4cccc6cccc(c46)O5)c4cccc5oc6ccccc6c45)c23)cc1 ; c1ccc(-c2cccc3cccc(-c4ccccc4N(c4cccc(-c5ccc6c(c5)-c5cccc7cccc(c57)O6)c4)c4cccc5oc6ccccc6c45)c23)cc1]

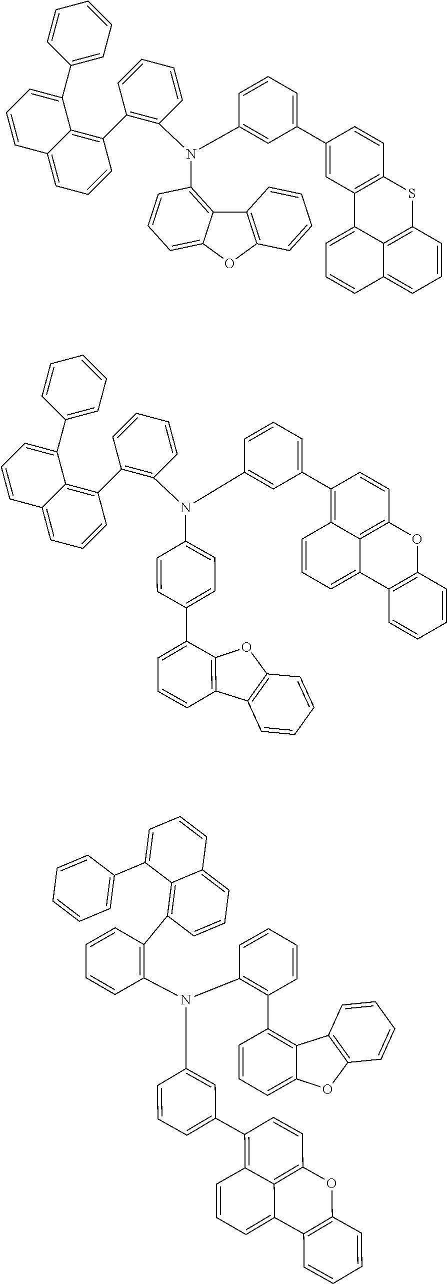 [SMILES: c1ccc(-c2cccc3cccc(-c4ccccc4N(c4cccc(-c5ccc6c(c5)-c5cccc7cccc(c57)S6)c4)c4cccc5oc6ccccc6c45)c23)cc1 ; c1ccc(-c2cccc3cccc(-c4ccccc4N(c4ccc(-c5cccc6c5oc5ccccc56)cc4)c4cccc(-c5ccc6c7c(cccc57)-c5ccccc5O6)c4)c23)cc1 ; c1ccc(-c2cccc3cccc(-c4ccccc4N(c4cccc(-c5ccc6c7c(cccc57)-c5ccccc5O6)c4)c4ccccc4-c4cccc5oc6ccccc6c45)c23)cc1]

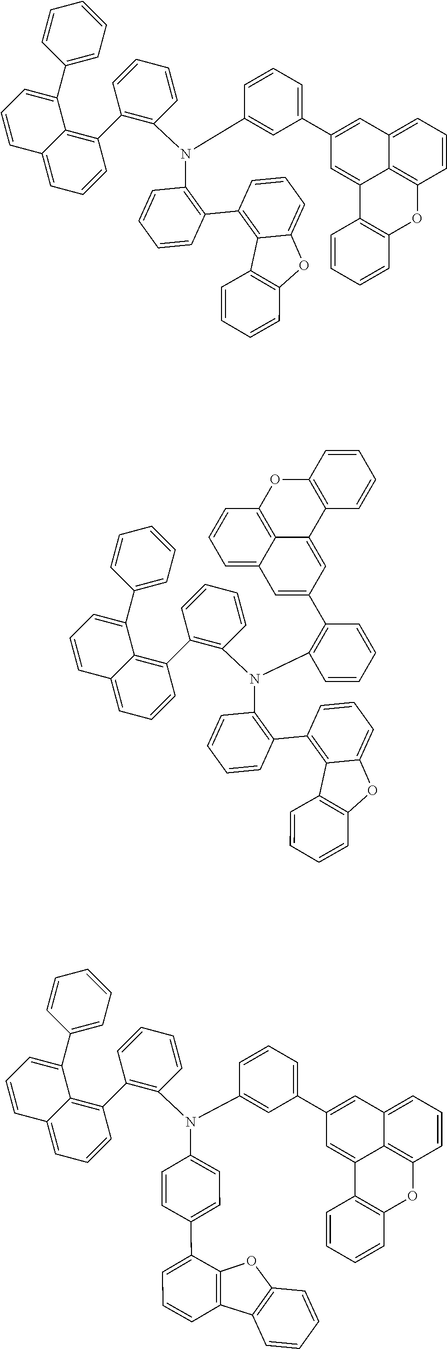 [SMILES: c1ccc(-c2cccc3cccc(-c4ccccc4N(c4cccc(-c5cc6c7c(cccc7c5)Oc5ccccc5-6)c4)c4ccccc4-c4cccc5oc6ccccc6c45)c23)cc1 ; c1ccc(-c2cccc3cccc(-c4ccccc4N(c4ccccc4-c4cc5c6c(cccc6c4)Oc4ccccc4-5)c4ccccc4-c4cccc5oc6ccccc6c45)c23)cc1 ; c1ccc(-c2cccc3cccc(-c4ccccc4N(c4ccc(-c5cccc6c5oc5ccccc56)cc4)c4cccc(-c5cc6c7c(cccc7c5)Oc5ccccc5-6)c4)c23)cc1]

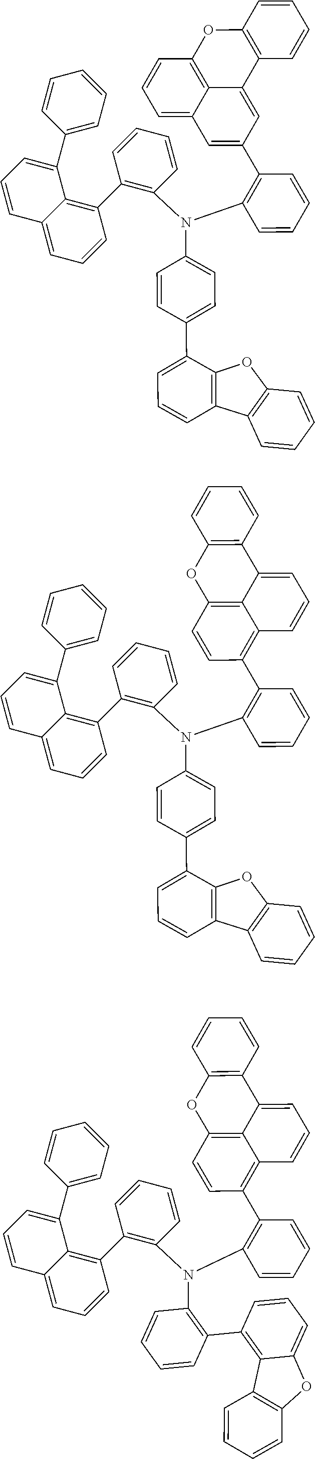

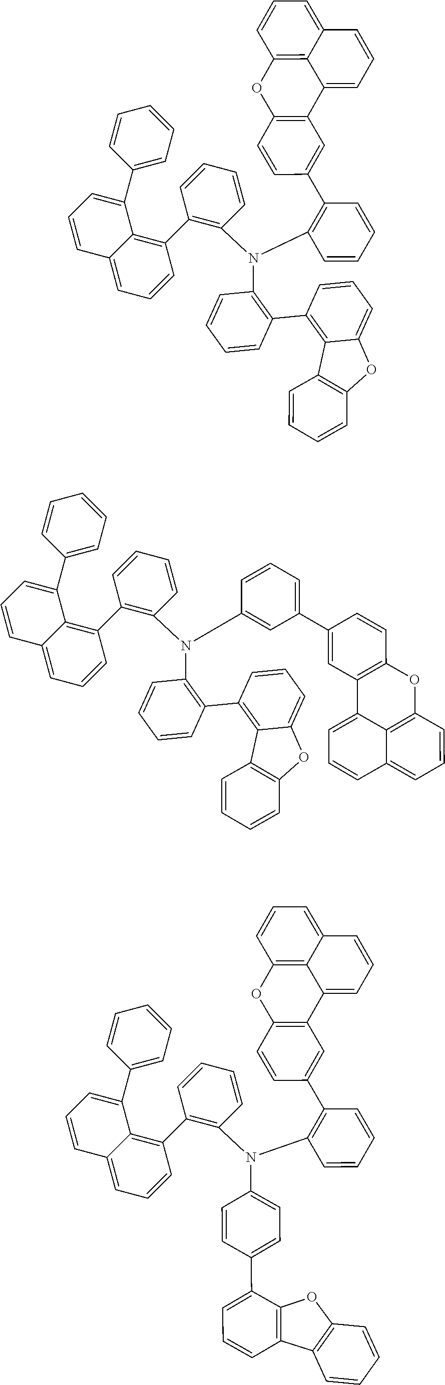 [SMILES: c1ccc(-c2cccc3cccc(-c4ccccc4N(c4ccccc4-c4ccc5c(c4)-c4cccc6cccc(c46)O5)c4ccccc4-c4cccc5oc6ccccc6c45)c23)cc1 ; c1ccc(-c2cccc3cccc(-c4ccccc4N(c4cccc(-c5ccc6c(c5)-c5cccc7cccc(c57)O6)c4)c4ccccc4-c4cccc5oc6ccccc6c45)c23)cc1 ; c1ccc(-c2cccc3cccc(-c4ccccc4N(c4ccc(-c5cccc6c5oc5ccccc56)cc4)c4ccccc4-c4ccc5c(c4)-c4cccc6cccc(c46)O5)c23)cc1]

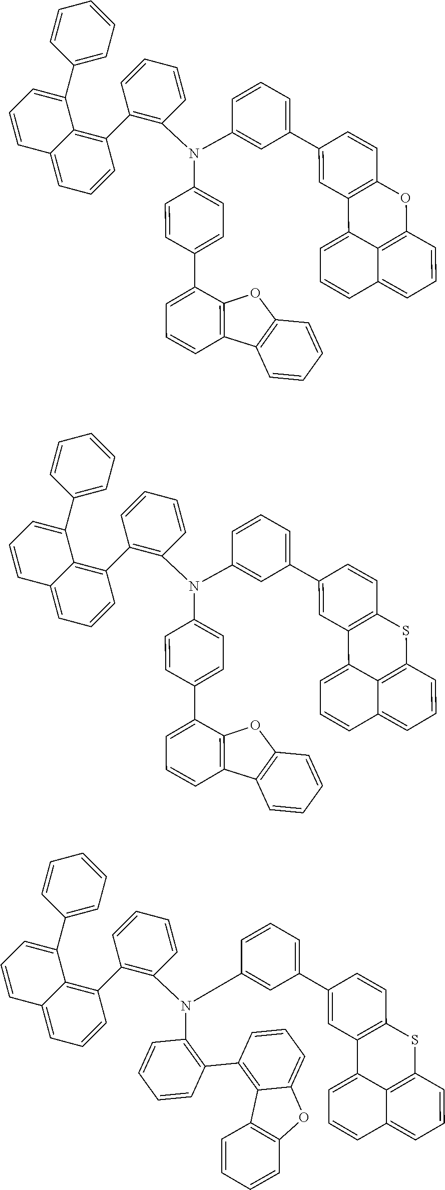 [SMILES: c1ccc(-c2cccc3cccc(-c4ccccc4N(c4ccc(-c5cccc6c5oc5ccccc56)cc4)c4cccc(-c5ccc6c(c5)-c5cccc7cccc(c57)O6)c4)c23)cc1 ; c1ccc(-c2cccc3cccc(-c4ccccc4N(c4ccc(-c5cccc6c5oc5ccccc56)cc4)c4cccc(-c5ccc6c(c5)-c5cccc7cccc(c57)S6)c4)c23)cc1 ; c1ccc(-c2cccc3cccc(-c4ccccc4N(c4cccc(-c5ccc6c(c5)-c5cccc7cccc(c57)S6)c4)c4ccccc4-c4cccc5oc6ccccc6c45)c23)cc1]

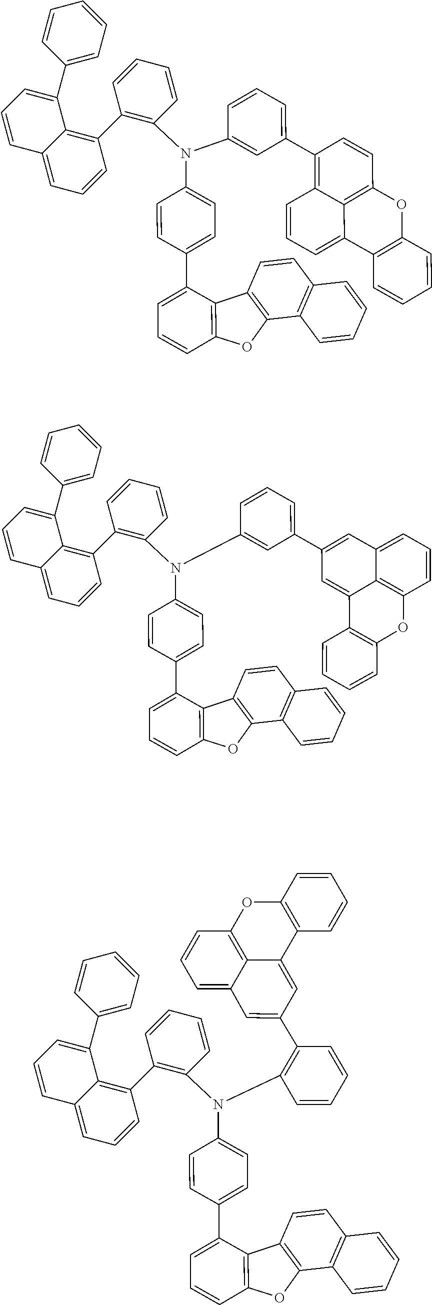 [SMILES: c1ccc(-c2cccc3cccc(-c4ccccc4N(c4ccc(-c5cccc6oc7c8ccccc8ccc7c56)cc4)c4cccc(-c5ccc6c7c(cccc57)-c5ccccc5O6)c4)c23)cc1 ; c1ccc(-c2cccc3cccc(-c4ccccc4N(c4ccc(-c5cccc6oc7c8ccccc8ccc7c56)cc4)c4cccc(-c5cc6c7c(cccc7c5)Oc5ccccc5-6)c4)c23)cc1 ; c1ccc(-c2cccc3cccc(-c4ccccc4N(c4ccc(-c5cccc6oc7c8ccccc8ccc7c56)cc4)c4ccccc4-c4cc5c6c(cccc6c4)Oc4ccccc4-5)c23)cc1]

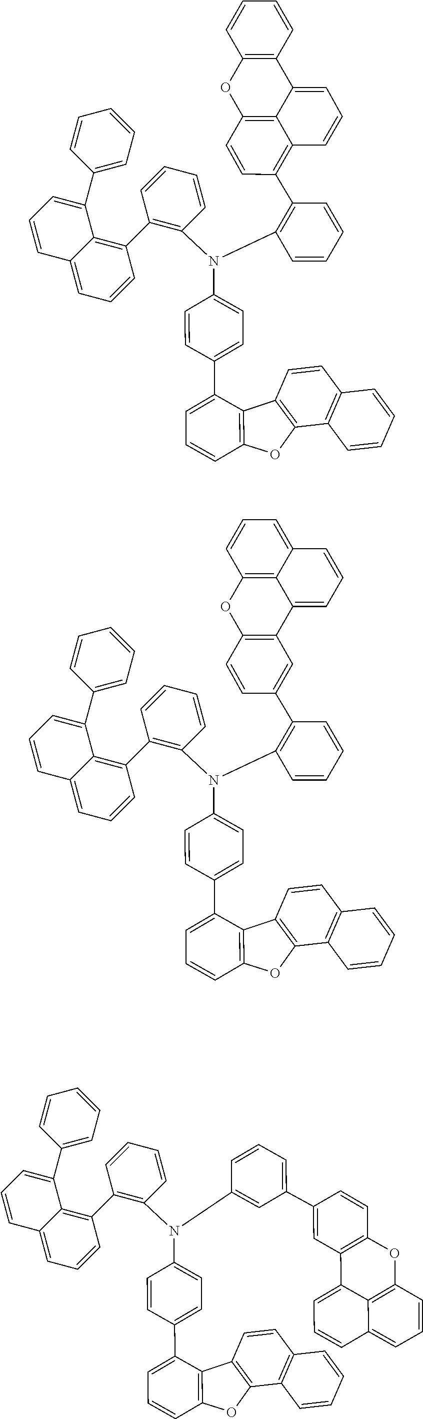 [SMILES: c1ccc(-c2cccc3cccc(-c4ccccc4N(c4ccc(-c5cccc6oc7c8ccccc8ccc7c56)cc4)c4ccccc4-c4ccc5c6c(cccc46)-c4ccccc4O5)c23)cc1 ; c1ccc(-c2cccc3cccc(-c4ccccc4N(c4ccc(-c5cccc6oc7c8ccccc8ccc7c56)cc4)c4ccccc4-c4ccc5c(c4)-c4cccc6cccc(c46)O5)c23)cc1 ; c1ccc(-c2cccc3cccc(-c4ccccc4N(c4ccc(-c5cccc6oc7c8ccccc8ccc7c56)cc4)c4cccc(-c5ccc6c(c5)-c5cccc7cccc(c57)O6)c4)c23)cc1]

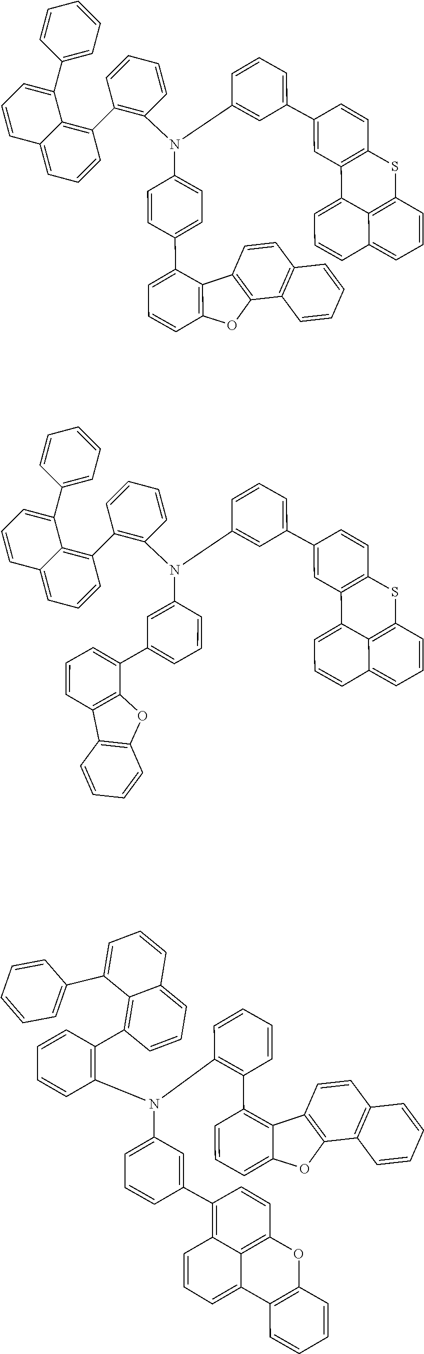 [SMILES: c1ccc(-c2cccc3cccc(-c4ccccc4N(c4ccc(-c5cccc6oc7c8ccccc8ccc7c56)cc4)c4cccc(-c5ccc6c(c5)-c5cccc7cccc(c57)S6)c4)c23)cc1 ; c1ccc(-c2cccc3cccc(-c4ccccc4N(c4cccc(-c5ccc6c(c5)-c5cccc7cccc(c57)S6)c4)c4cccc(-c5cccc6c5oc5ccccc56)c4)c23)cc1 ; c1ccc(-c2cccc3cccc(-c4ccccc4N(c4cccc(-c5ccc6c7c(cccc57)-c5ccccc5O6)c4)c4ccccc4-c4cccc5oc6c7ccccc7ccc6c45)c23)cc1]

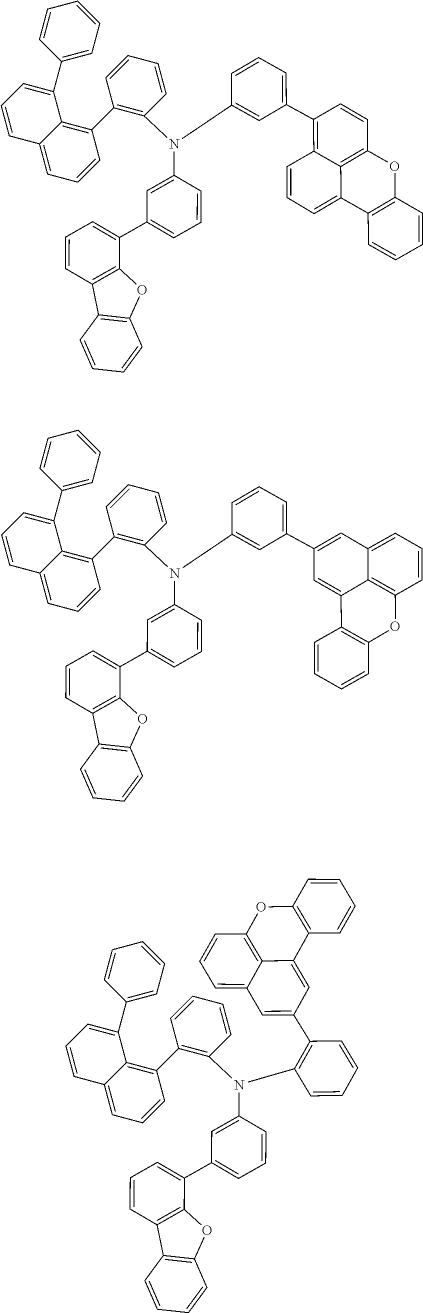 [SMILES: c1ccc(-c2cccc3cccc(-c4ccccc4N(c4cccc(-c5ccc6c7c(cccc57)-c5ccccc5O6)c4)c4cccc(-c5cccc6c5oc5ccccc56)c4)c23)cc1 ; c1ccc(-c2cccc3cccc(-c4ccccc4N(c4cccc(-c5cc6c7c(cccc7c5)Oc5ccccc5-6)c4)c4cccc(-c5cccc6c5oc5ccccc56)c4)c23)cc1 ; c1ccc(-c2cccc3cccc(-c4ccccc4N(c4cccc(-c5cccc6c5oc5ccccc56)c4)c4ccccc4-c4cc5c6c(cccc6c4)Oc4ccccc4-5)c23)cc1]

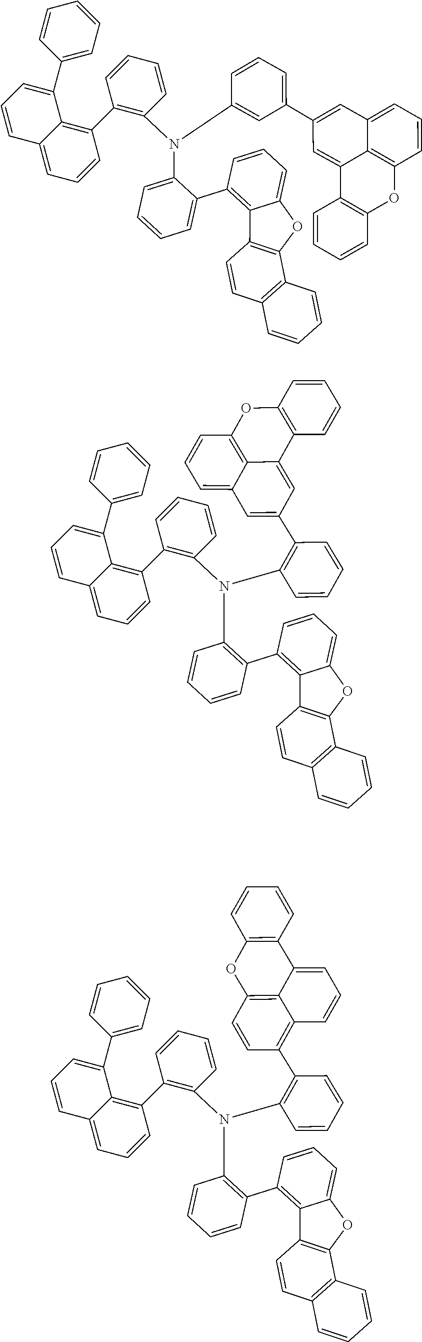

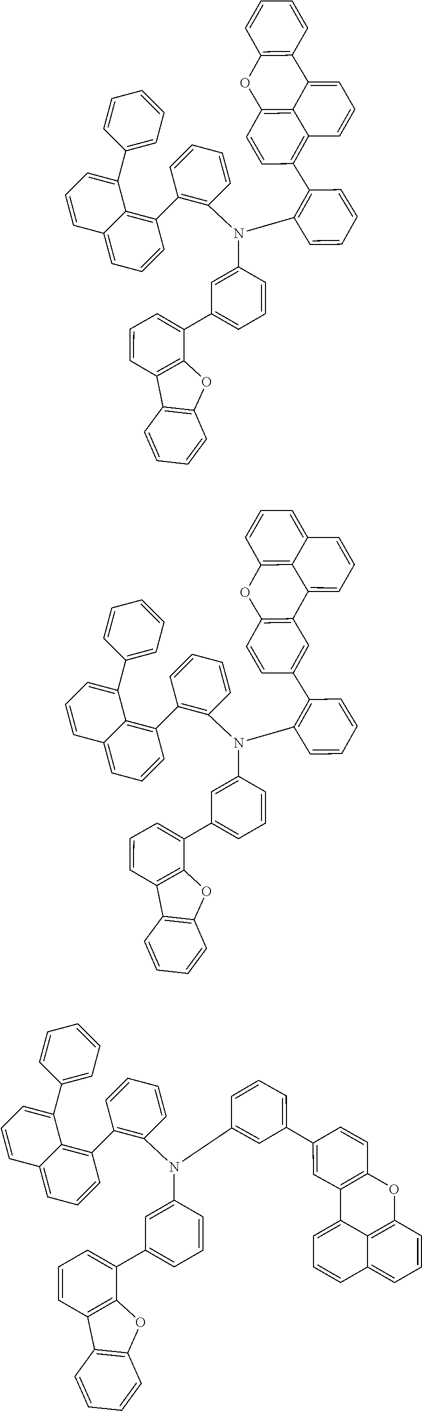 [SMILES: c1ccc(-c2cccc3cccc(-c4ccccc4N(c4cccc(-c5cccc6c5oc5ccccc56)c4)c4ccccc4-c4ccc5c6c(cccc46)-c4ccccc4O5)c23)cc1 ; c1ccc(-c2cccc3cccc(-c4ccccc4N(c4cccc(-c5cccc6c5oc5ccccc56)c4)c4ccccc4-c4ccc5c(c4)-c4cccc6cccc(c46)O5)c23)cc1 ; c1ccc(-c2cccc3cccc(-c4ccccc4N(c4cccc(-c5ccc6c(c5)-c5cccc7cccc(c57)O6)c4)c4cccc(-c5cccc6c5oc5ccccc56)c4)c23)cc1]

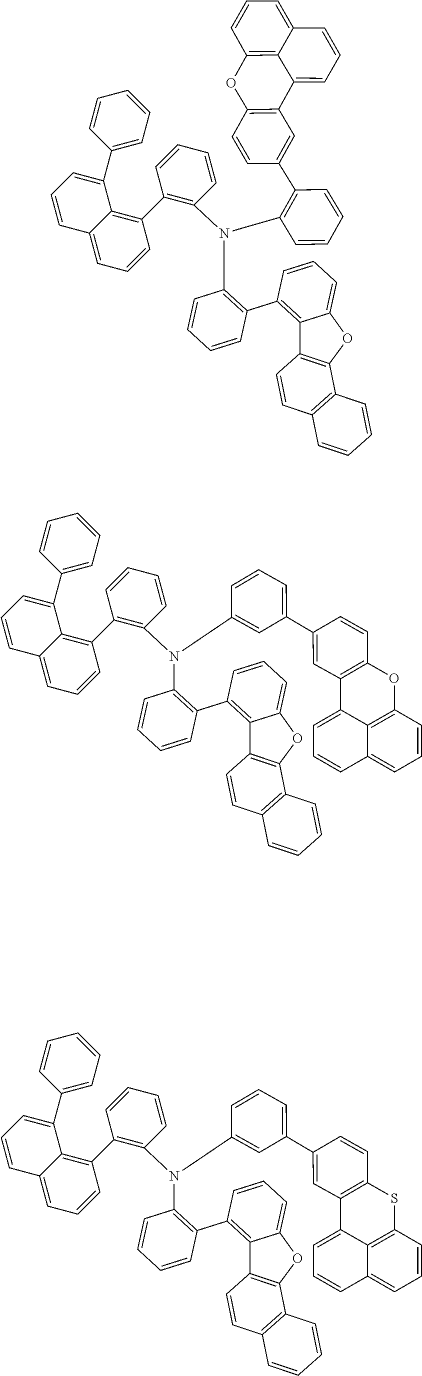 [SMILES: c1ccc(-c2cccc3cccc(-c4ccccc4N(c4ccccc4-c4ccc5c(c4)-c4cccc6cccc(c46)O5)c4ccccc4-c4cccc5oc6c7ccccc7ccc6c45)c23)cc1 ; c1ccc(-c2cccc3cccc(-c4ccccc4N(c4cccc(-c5ccc6c(c5)-c5cccc7cccc(c57)O6)c4)c4ccccc4-c4cccc5oc6c7ccccc7ccc6c45)c23)cc1 ; c1ccc(-c2cccc3cccc(-c4ccccc4N(c4cccc(-c5ccc6c(c5)-c5cccc7cccc(c57)S6)c4)c4ccccc4-c4cccc5oc6c7ccccc7ccc6c45)c23)cc1]

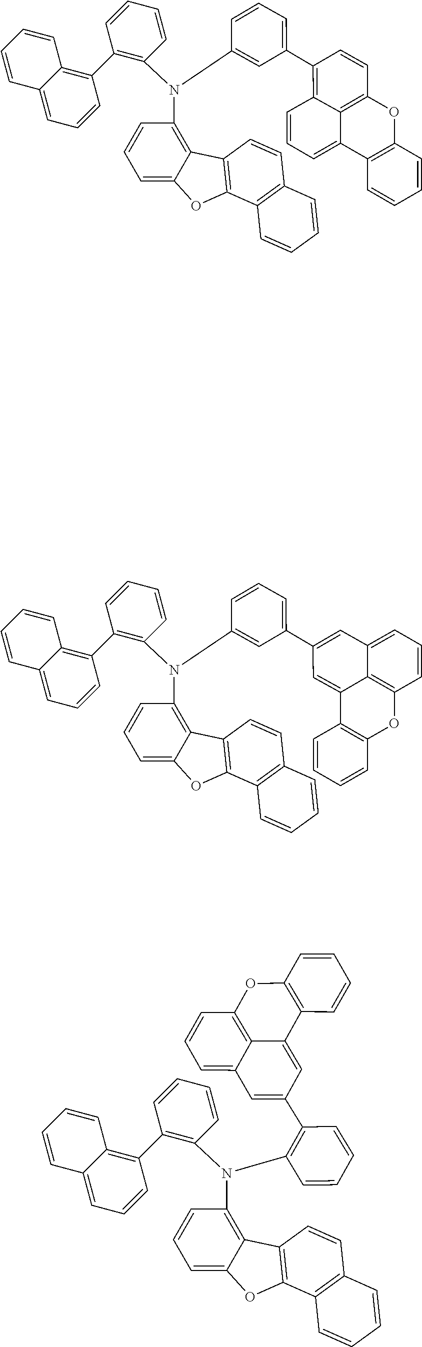 [SMILES: c1cc(-c2ccc3c4c(cccc24)-c2ccccc2O3)cc(N(c2ccccc2-c2cccc3ccccc23)c2cccc3oc4c5ccccc5ccc4c23)c1 ; c1cc(-c2cc3c4c(cccc4c2)Oc2ccccc2-3)cc(N(c2ccccc2-c2cccc3ccccc23)c2cccc3oc4c5ccccc5ccc4c23)c1 ; c1ccc2c(c1)Oc1cccc3cc(-c4ccccc4N(c4ccccc4-c4cccc5ccccc45)c4cccc5oc6c7ccccc7ccc6c45)cc-2c13]

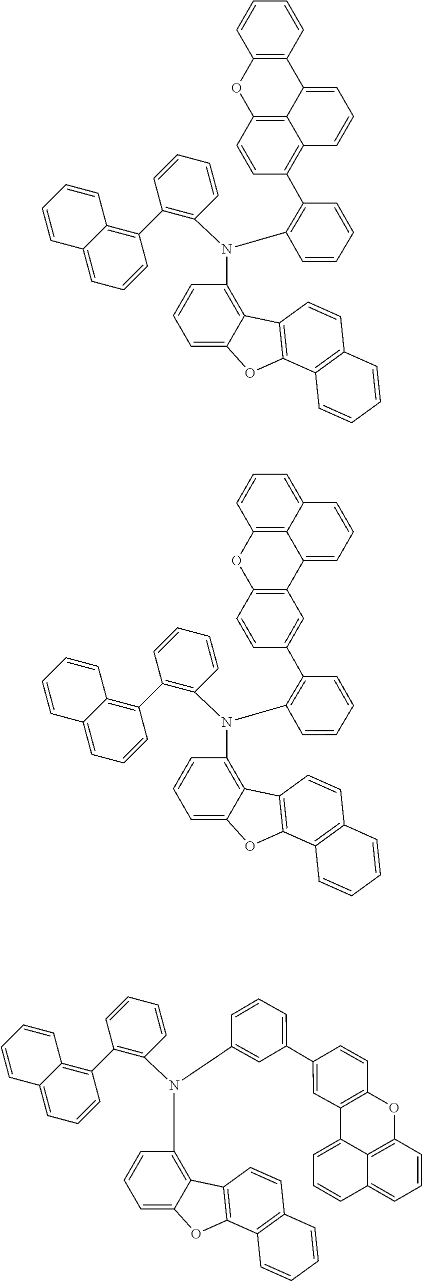 [SMILES: c1ccc2c(c1)Oc1ccc(-c3ccccc3N(c3ccccc3-c3cccc4ccccc34)c3cccc4oc5c6ccccc6ccc5c34)c3cccc-2c13 ; c1ccc(N(c2ccccc2-c2cccc3ccccc23)c2cccc3oc4c5ccccc5ccc4c23)c(-c2ccc3c(c2)-c2cccc4cccc(c24)O3)c1 ; c1cc(-c2ccc3c(c2)-c2cccc4cccc(c24)O3)cc(N(c2ccccc2-c2cccc3ccccc23)c2cccc3oc4c5ccccc5ccc4c23)c1]

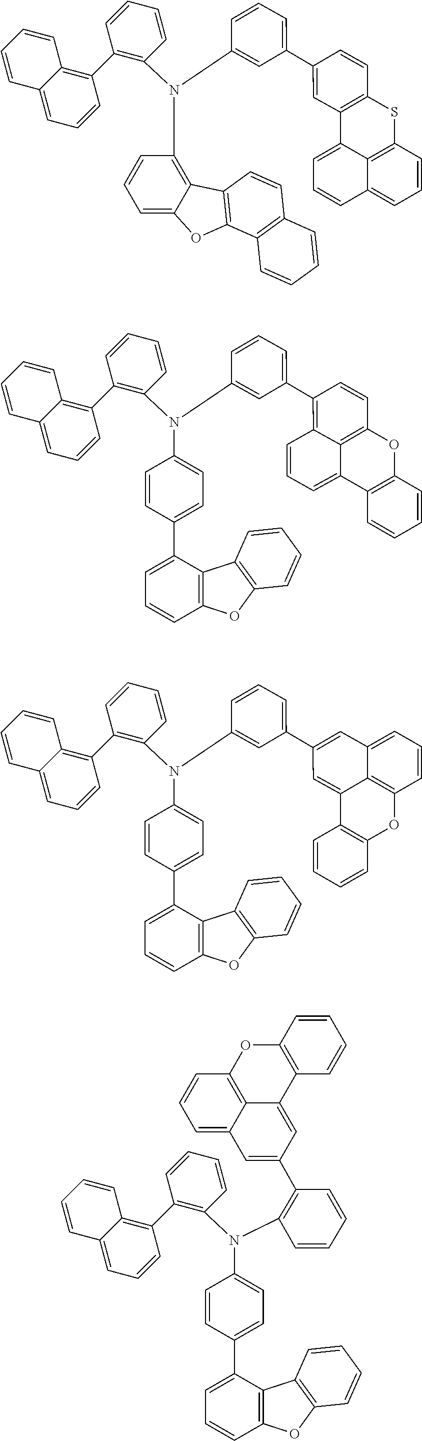 [SMILES: c1cc(-c2ccc3c(c2)-c2cccc4cccc(c24)S3)cc(N(c2ccccc2-c2cccc3ccccc23)c2cccc3oc4c5ccccc5ccc4c23)c1 ; c1cc(-c2ccc3c4c(cccc24)-c2ccccc2O3)cc(N(c2ccc(-c3cccc4oc5ccccc5c34)cc2)c2ccccc2-c2cccc3ccccc23)c1 ; c1cc(-c2cc3c4c(cccc4c2)Oc2ccccc2-3)cc(N(c2ccc(-c3cccc4oc5ccccc5c34)cc2)c2ccccc2-c2cccc3ccccc23)c1 ; c1ccc2c(c1)Oc1cccc3cc(-c4ccccc4N(c4ccc(-c5cccc6oc7ccccc7c56)cc4)c4ccccc4-c4cccc5ccccc45)cc-2c13]

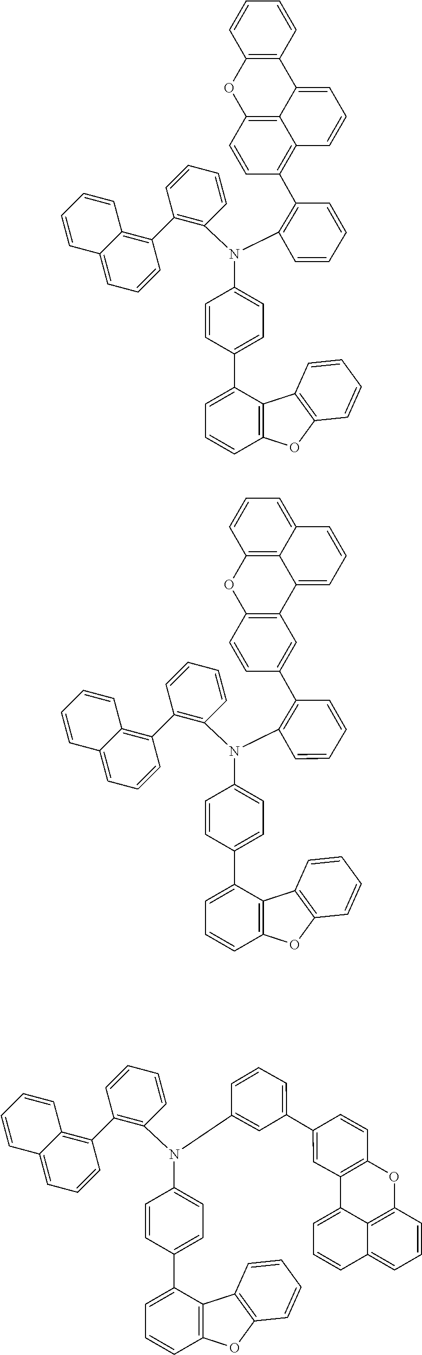

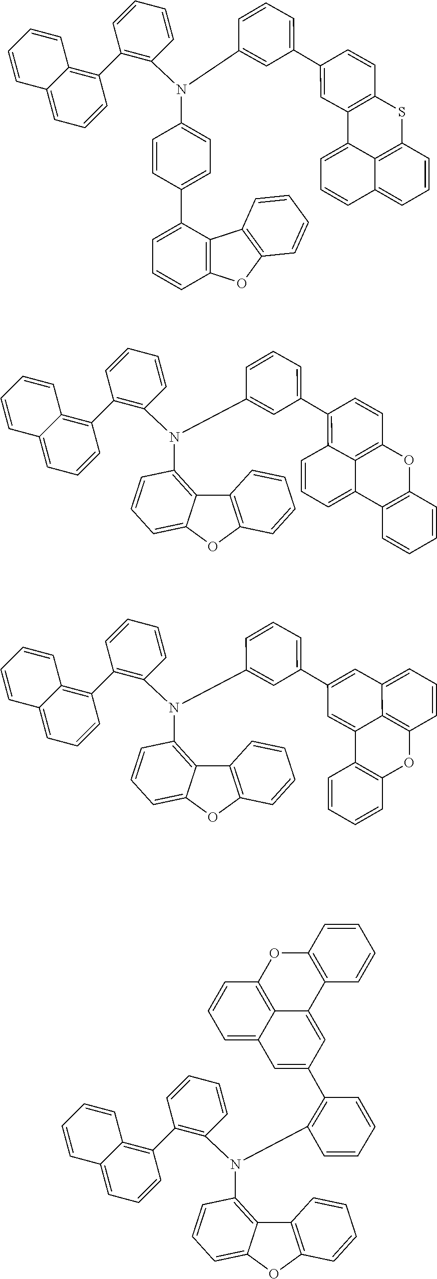 [SMILES: c1cc(-c2ccc3c(c2)-c2cccc4cccc(c24)S3)cc(N(c2ccc(-c3cccc4oc5ccccc5c34)cc2)c2ccccc2-c2cccc3ccccc23)c1 ; c1cc(-c2ccc3c4c(cccc24)-c2ccccc2O3)cc(N(c2ccccc2-c2cccc3ccccc23)c2cccc3oc4ccccc4c23)c1 ; c1cc(-c2cc3c4c(cccc4c2)Oc2ccccc2-3)cc(N(c2ccccc2-c2cccc3ccccc23)c2cccc3oc4ccccc4c23)c1 ; c1ccc2c(c1)Oc1cccc3cc(-c4ccccc4N(c4ccccc4-c4cccc5ccccc45)c4cccc5oc6ccccc6c45)cc-2c13]

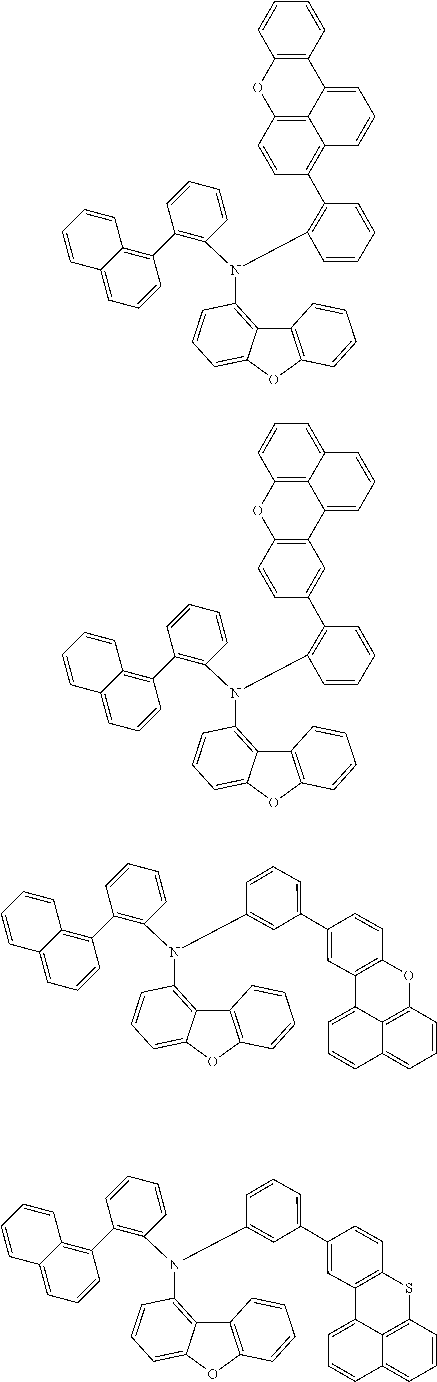

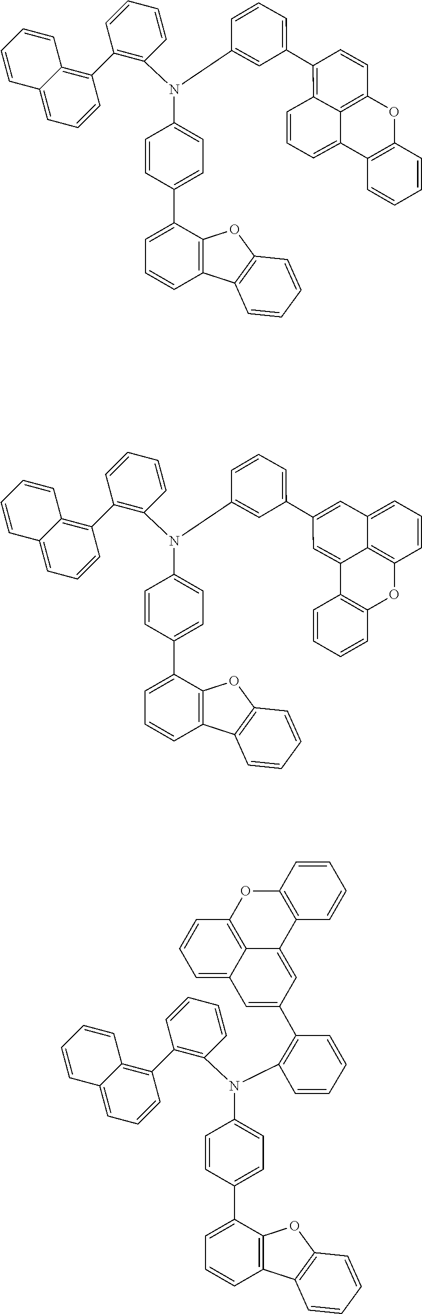 [SMILES: c1cc(-c2ccc3c4c(cccc24)-c2ccccc2O3)cc(N(c2ccc(-c3cccc4c3oc3ccccc34)cc2)c2ccccc2-c2cccc3ccccc23)c1 ; c1cc(-c2cc3c4c(cccc4c2)Oc2ccccc2-3)cc(N(c2ccc(-c3cccc4c3oc3ccccc34)cc2)c2ccccc2-c2cccc3ccccc23)c1 ; c1ccc2c(c1)Oc1cccc3cc(-c4ccccc4N(c4ccc(-c5cccc6c5oc5ccccc56)cc4)c4ccccc4-c4cccc5ccccc45)cc-2c13]

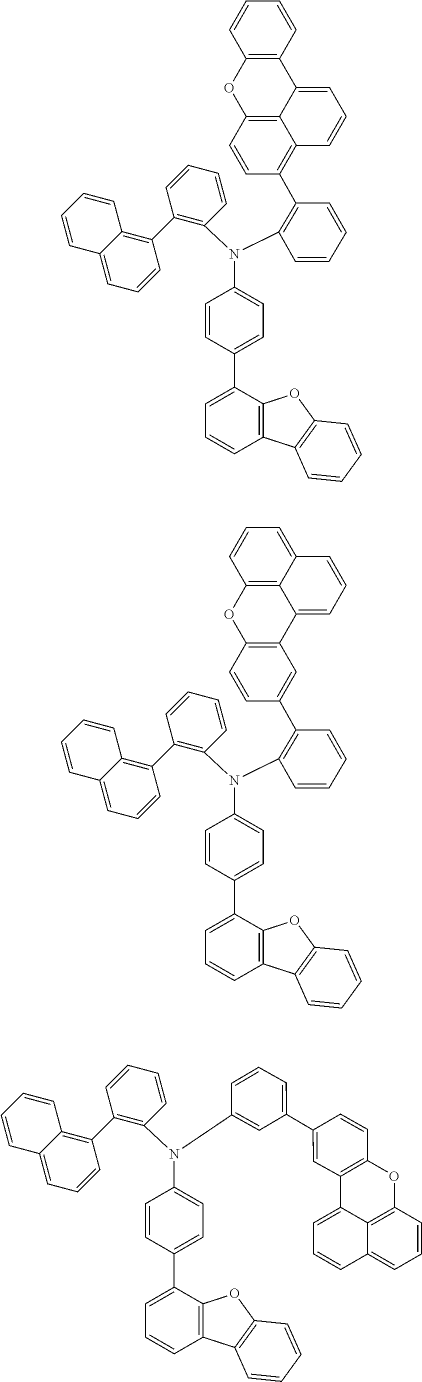 [SMILES: c1ccc2c(c1)Oc1ccc(-c3ccccc3N(c3ccc(-c4cccc5c4oc4ccccc45)cc3)c3ccccc3-c3cccc4ccccc34)c3cccc-2c13 ; c1ccc(N(c2ccc(-c3cccc4c3oc3ccccc34)cc2)c2ccccc2-c2cccc3ccccc23)c(-c2ccc3c(c2)-c2cccc4cccc(c24)O3)c1 ; c1cc(-c2ccc3c(c2)-c2cccc4cccc(c24)O3)cc(N(c2ccc(-c3cccc4c3oc3ccccc34)cc2)c2ccccc2-c2cccc3ccccc23)c1]

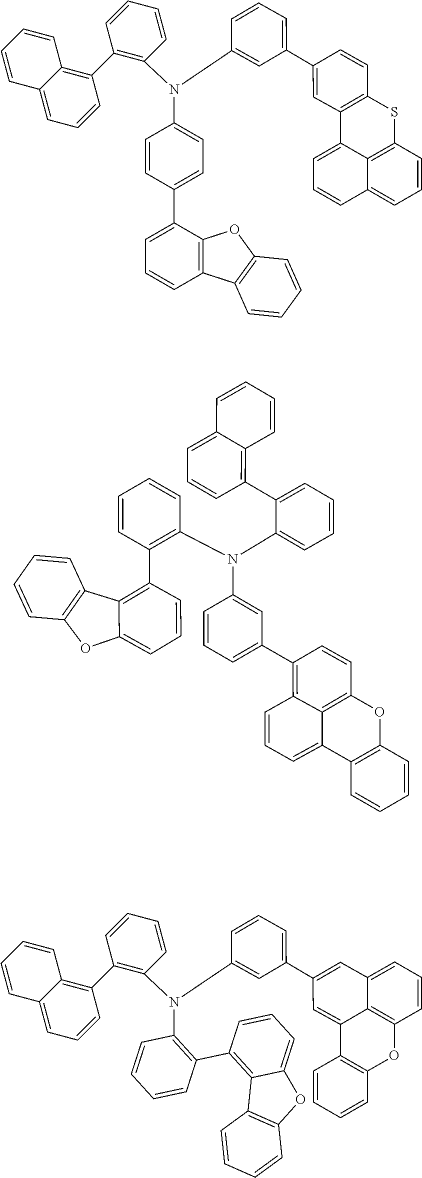 [SMILES: c1cc(-c2ccc3c(c2)-c2cccc4cccc(c24)S3)cc(N(c2ccc(-c3cccc4c3oc3ccccc34)cc2)c2ccccc2-c2cccc3ccccc23)c1 ; c1cc(-c2ccc3c4c(cccc24)-c2ccccc2O3)cc(N(c2ccccc2-c2cccc3ccccc23)c2ccccc2-c2cccc3oc4ccccc4c23)c1 ; c1cc(-c2cc3c4c(cccc4c2)Oc2ccccc2-3)cc(N(c2ccccc2-c2cccc3ccccc23)c2ccccc2-c2cccc3oc4ccccc4c23)c1]

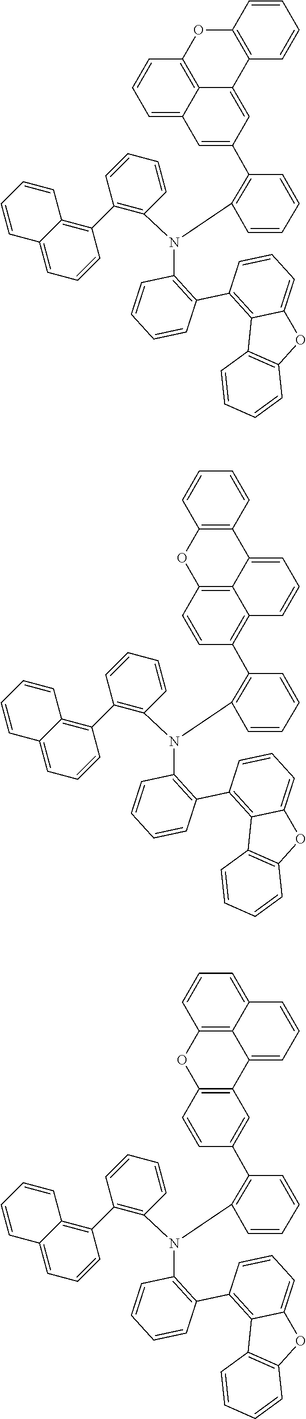 [SMILES: c1ccc2c(c1)Oc1cccc3cc(-c4ccccc4N(c4ccccc4-c4cccc5ccccc45)c4ccccc4-c4cccc5oc6ccccc6c45)cc-2c13 ; c1ccc2c(c1)Oc1ccc(-c3ccccc3N(c3ccccc3-c3cccc4ccccc34)c3ccccc3-c3cccc4oc5ccccc5c34)c3cccc-2c13 ; c1ccc(N(c2ccccc2-c2cccc3ccccc23)c2ccccc2-c2cccc3oc4ccccc4c23)c(-c2ccc3c(c2)-c2cccc4cccc(c24)O3)c1]

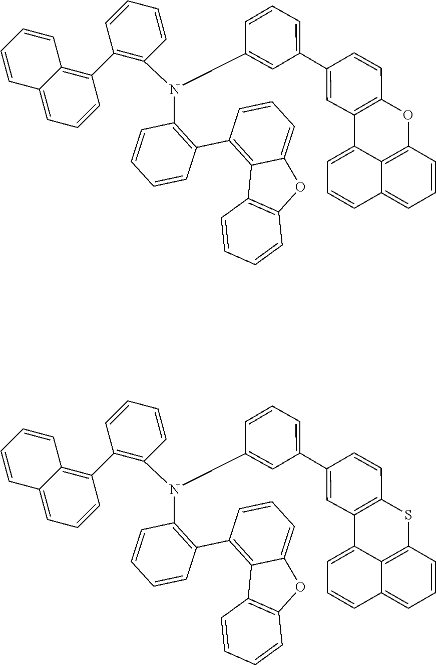 [SMILES: c1cc(-c2ccc3c(c2)-c2cccc4cccc(c24)O3)cc(N(c2ccccc2-c2cccc3ccccc23)c2ccccc2-c2cccc3oc4ccccc4c23)c1 ; c1cc(-c2ccc3c(c2)-c2cccc4cccc(c24)S3)cc(N(c2ccccc2-c2cccc3ccccc23)c2ccccc2-c2cccc3oc4ccccc4c23)c1]

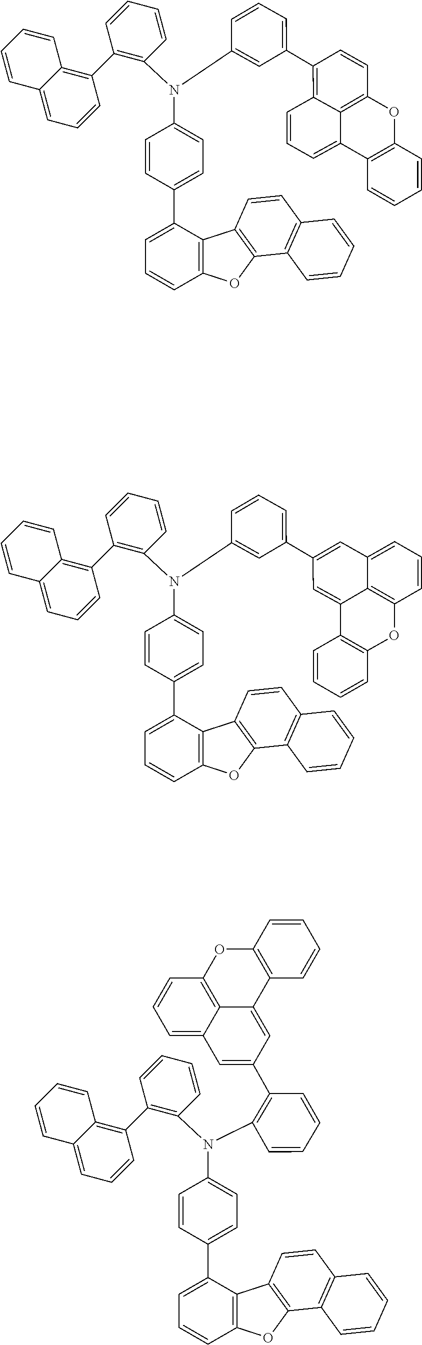 [SMILES: c1cc(-c2ccc3c4c(cccc24)-c2ccccc2O3)cc(N(c2ccc(-c3cccc4oc5c6ccccc6ccc5c34)cc2)c2ccccc2-c2cccc3ccccc23)c1 ; c1cc(-c2cc3c4c(cccc4c2)Oc2ccccc2-3)cc(N(c2ccc(-c3cccc4oc5c6ccccc6ccc5c34)cc2)c2ccccc2-c2cccc3ccccc23)c1 ; c1ccc2c(c1)Oc1cccc3cc(-c4ccccc4N(c4ccc(-c5cccc6oc7c8ccccc8ccc7c56)cc4)c4ccccc4-c4cccc5ccccc45)cc-2c13]

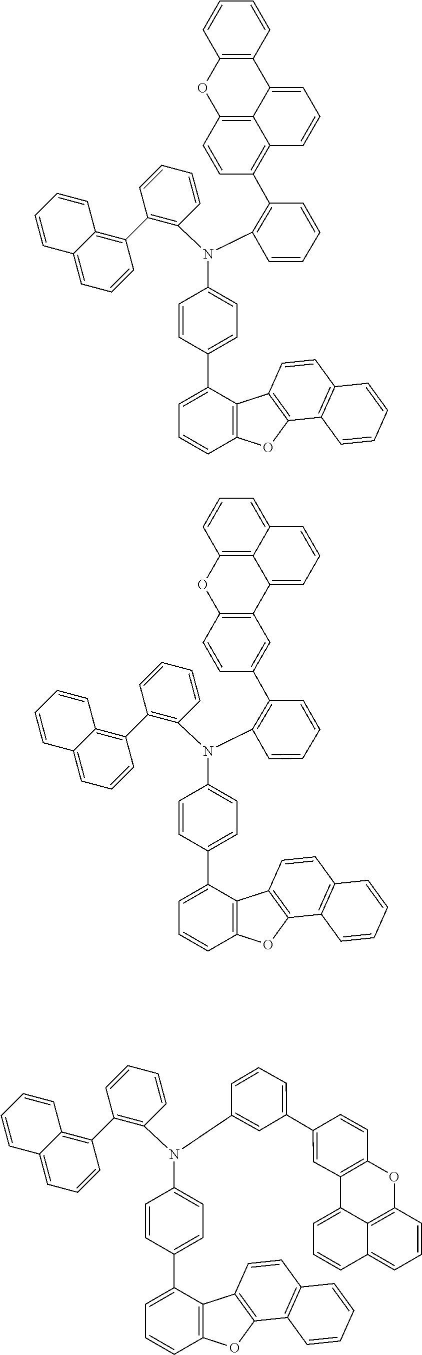

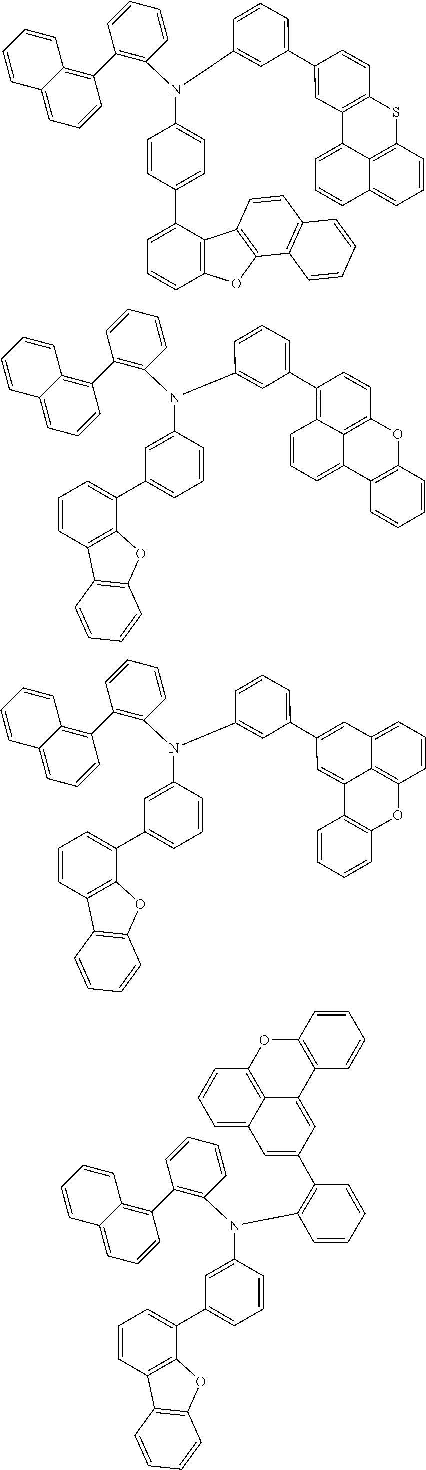

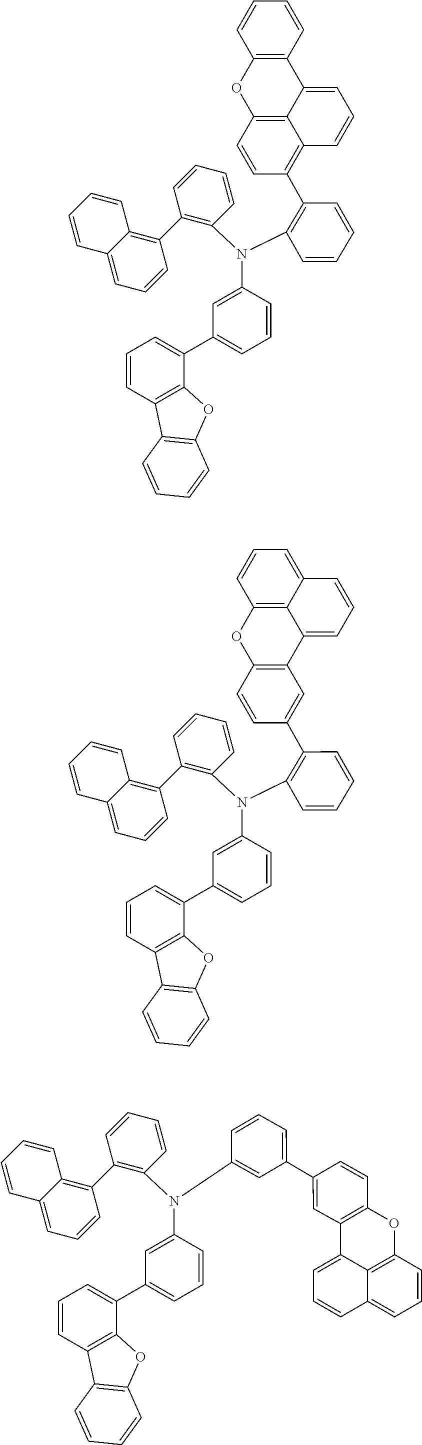 [SMILES: c1cc(-c2cccc3c2oc2ccccc23)cc(N(c2ccccc2-c2cccc3ccccc23)c2ccccc2-c2ccc3c4c(cccc24)-c2ccccc2O3)c1 ; c1cc(-c2cccc3c2oc2ccccc23)cc(N(c2ccccc2-c2ccc3c(c2)-c2cccc4cccc(c24)O3)c2ccccc2-c2cccc3ccccc23)c1 ; c1cc(-c2ccc3c(c2)-c2cccc4cccc(c24)O3)cc(N(c2cccc(-c3cccc4c3oc3ccccc34)c2)c2ccccc2-c2cccc3ccccc23)c1]

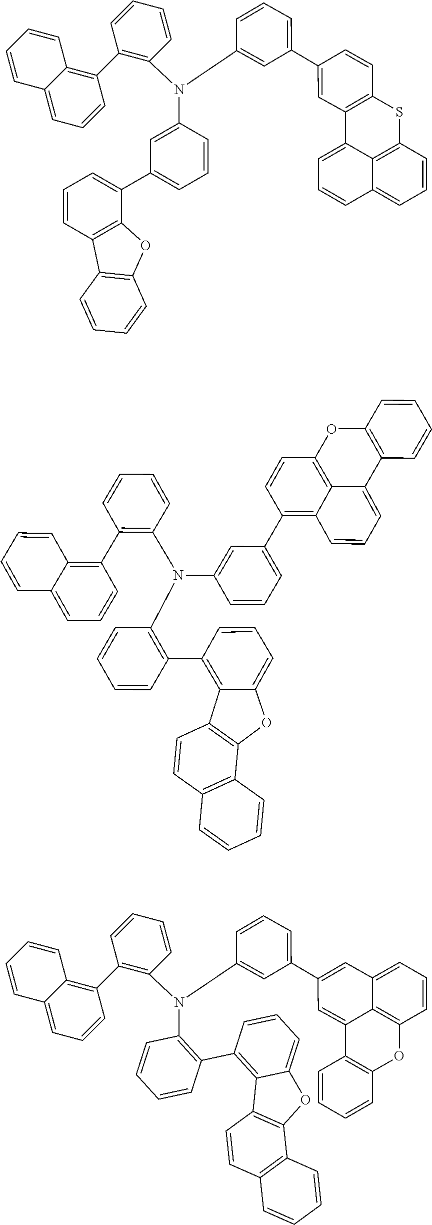 [SMILES: c1cc(-c2ccc3c(c2)-c2cccc4cccc(c24)S3)cc(N(c2cccc(-c3cccc4c3oc3ccccc34)c2)c2ccccc2-c2cccc3ccccc23)c1 ; c1cc(-c2ccc3c4c(cccc24)-c2ccccc2O3)cc(N(c2ccccc2-c2cccc3ccccc23)c2ccccc2-c2cccc3oc4c5ccccc5ccc4c23)c1 ; c1cc(-c2cc3c4c(cccc4c2)Oc2ccccc2-3)cc(N(c2ccccc2-c2cccc3ccccc23)c2ccccc2-c2cccc3oc4c5ccccc5ccc4c23)c1]

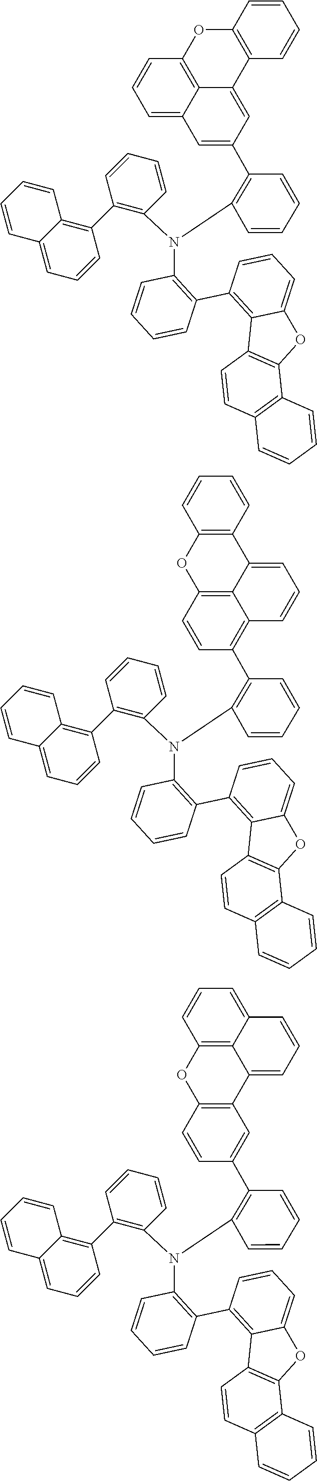

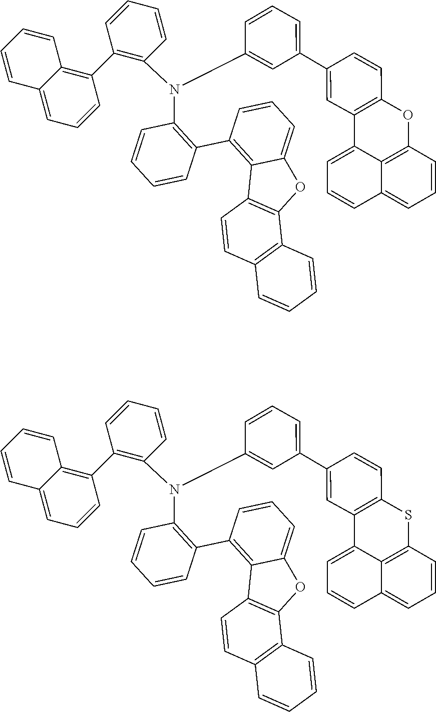 [SMILES: c1cc(-c2ccc3c(c2)-c2cccc4cccc(c24)O3)cc(N(c2ccccc2-c2cccc3ccccc23)c2ccccc2-c2cccc3oc4c5ccccc5ccc4c23)c1 ; c1cc(-c2ccc3c(c2)-c2cccc4cccc(c24)S3)cc(N(c2ccccc2-c2cccc3ccccc23)c2ccccc2-c2cccc3oc4c5ccccc5ccc4c23)c1]

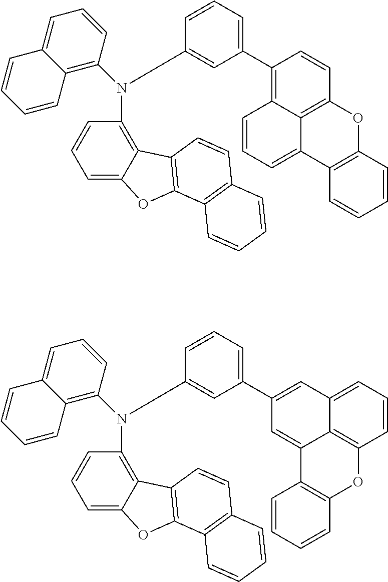 [SMILES: c1cc(-c2ccc3c4c(cccc24)-c2ccccc2O3)cc(N(c2cccc3ccccc23)c2cccc3oc4c5ccccc5ccc4c23)c1 ; c1cc(-c2cc3c4c(cccc4c2)Oc2ccccc2-3)cc(N(c2cccc3ccccc23)c2cccc3oc4c5ccccc5ccc4c23)c1]

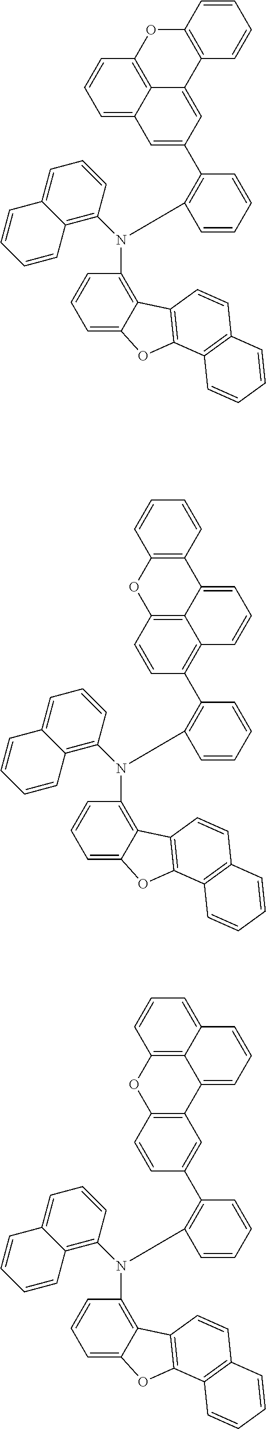 [SMILES: c1ccc2c(c1)Oc1cccc3cc(-c4ccccc4N(c4cccc5ccccc45)c4cccc5oc6c7ccccc7ccc6c45)cc-2c13 ; c1ccc2c(c1)Oc1ccc(-c3ccccc3N(c3cccc4ccccc34)c3cccc4oc5c6ccccc6ccc5c34)c3cccc-2c13 ; c1ccc(N(c2cccc3ccccc23)c2cccc3oc4c5ccccc5ccc4c23)c(-c2ccc3c(c2)-c2cccc4cccc(c24)O3)c1]

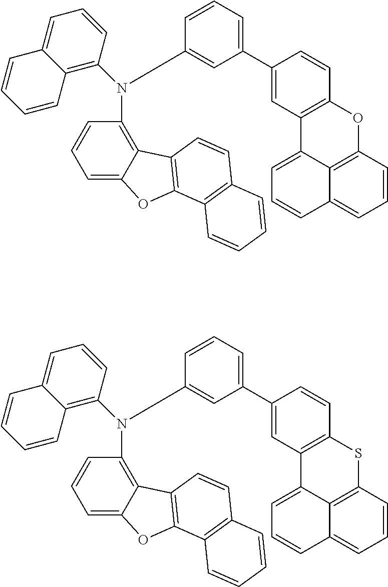 [SMILES: c1cc(-c2ccc3c(c2)-c2cccc4cccc(c24)O3)cc(N(c2cccc3ccccc23)c2cccc3oc4c5ccccc5ccc4c23)c1 ; c1cc(-c2ccc3c(c2)-c2cccc4cccc(c24)S3)cc(N(c2cccc3ccccc23)c2cccc3oc4c5ccccc5ccc4c23)c1]

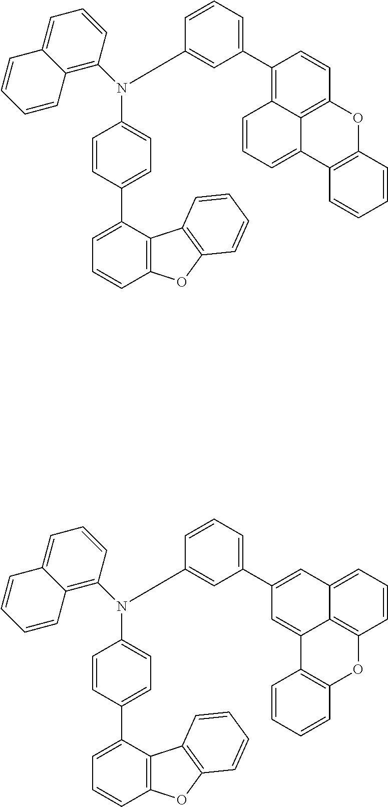 [SMILES: c1cc(-c2ccc3c4c(cccc24)-c2ccccc2O3)cc(N(c2ccc(-c3cccc4oc5ccccc5c34)cc2)c2cccc3ccccc23)c1 ; c1cc(-c2cc3c4c(cccc4c2)Oc2ccccc2-3)cc(N(c2ccc(-c3cccc4oc5ccccc5c34)cc2)c2cccc3ccccc23)c1]

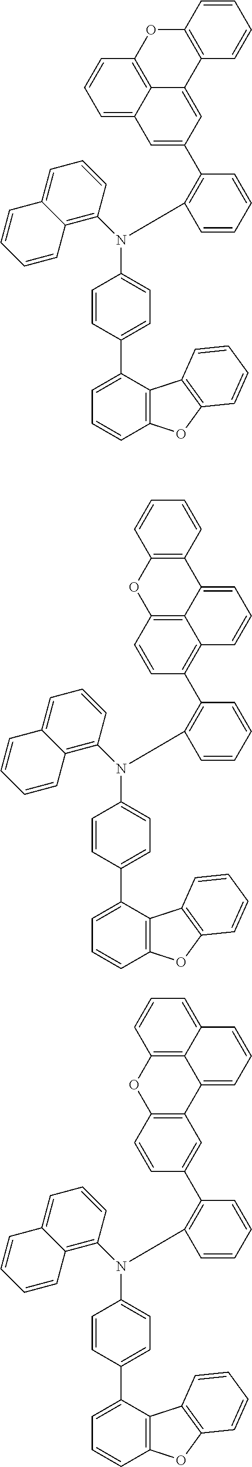

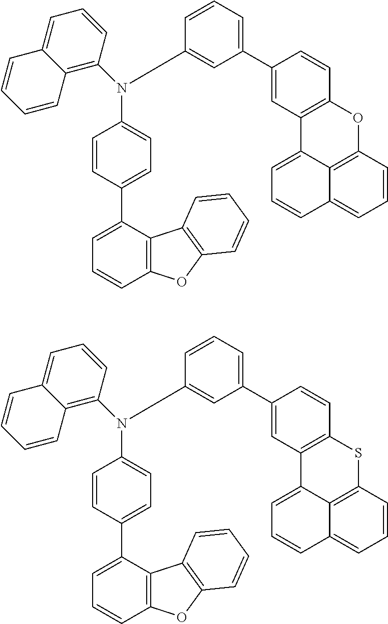 [SMILES: c1cc(-c2ccc3c(c2)-c2cccc4cccc(c24)O3)cc(N(c2ccc(-c3cccc4oc5ccccc5c34)cc2)c2cccc3ccccc23)c1 ; c1cc(-c2ccc3c(c2)-c2cccc4cccc(c24)S3)cc(N(c2ccc(-c3cccc4oc5ccccc5c34)cc2)c2cccc3ccccc23)c1]

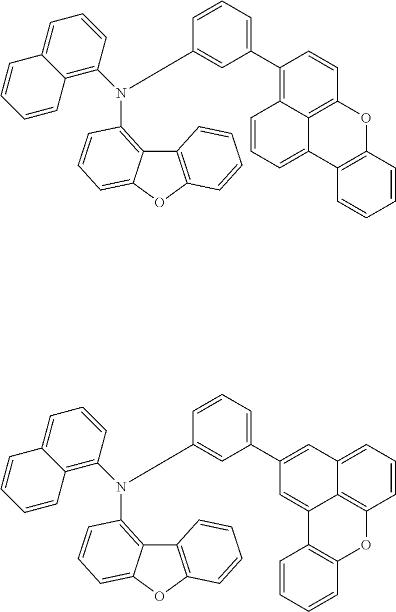 [SMILES: c1cc(-c2ccc3c4c(cccc24)-c2ccccc2O3)cc(N(c2cccc3ccccc23)c2cccc3oc4ccccc4c23)c1 ; c1cc(-c2cc3c4c(cccc4c2)Oc2ccccc2-3)cc(N(c2cccc3ccccc23)c2cccc3oc4ccccc4c23)c1]

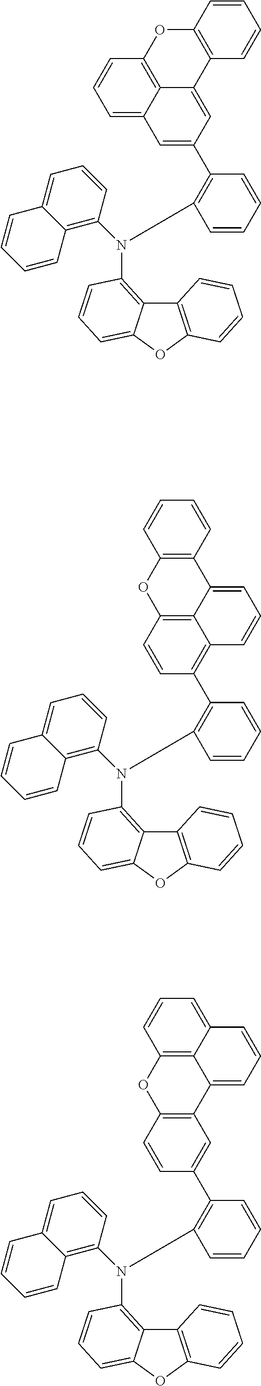 [SMILES: c1ccc2c(c1)Oc1cccc3cc(-c4ccccc4N(c4cccc5ccccc45)c4cccc5oc6ccccc6c45)cc-2c13 ; c1ccc2c(c1)Oc1ccc(-c3ccccc3N(c3cccc4ccccc34)c3cccc4oc5ccccc5c34)c3cccc-2c13 ; c1ccc(N(c2cccc3ccccc23)c2cccc3oc4ccccc4c23)c(-c2ccc3c(c2)-c2cccc4cccc(c24)O3)c1]

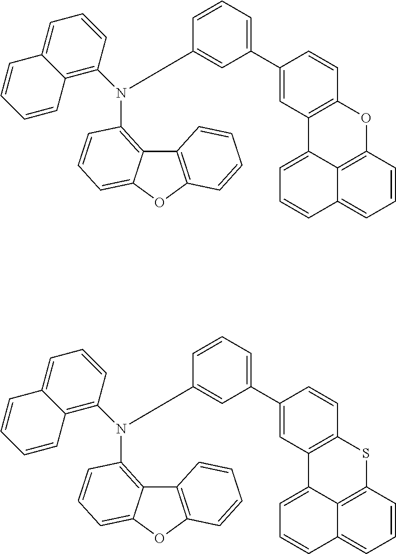 [SMILES: c1cc(-c2ccc3c(c2)-c2cccc4cccc(c24)O3)cc(N(c2cccc3ccccc23)c2cccc3oc4ccccc4c23)c1 ; c1cc(-c2ccc3c(c2)-c2cccc4cccc(c24)S3)cc(N(c2cccc3ccccc23)c2cccc3oc4ccccc4c23)c1]

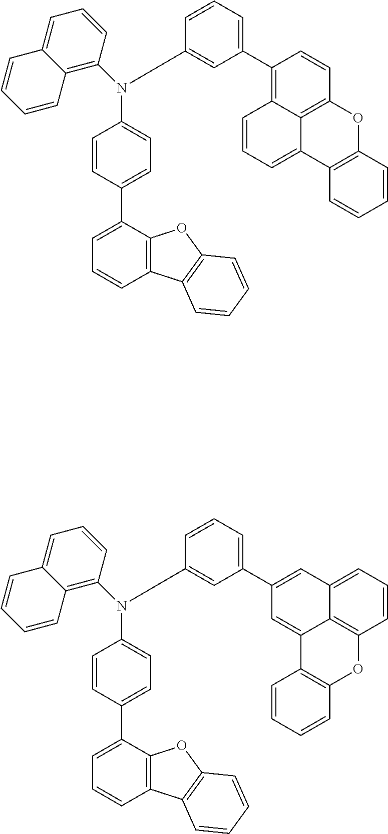 [SMILES: c1cc(-c2ccc3c4c(cccc24)-c2ccccc2O3)cc(N(c2ccc(-c3cccc4c3oc3ccccc34)cc2)c2cccc3ccccc23)c1 ; c1cc(-c2cc3c4c(cccc4c2)Oc2ccccc2-3)cc(N(c2ccc(-c3cccc4c3oc3ccccc34)cc2)c2cccc3ccccc23)c1]

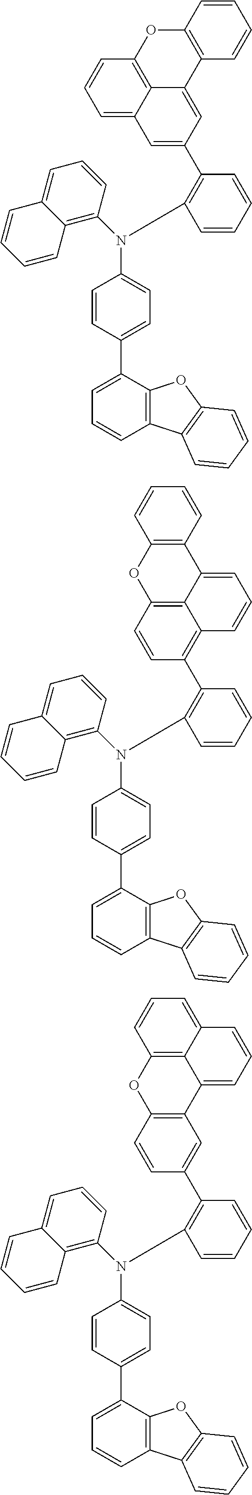

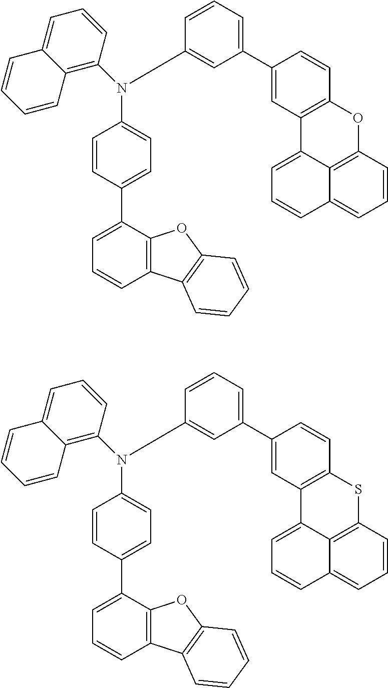 [SMILES: c1cc(-c2ccc3c(c2)-c2cccc4cccc(c24)O3)cc(N(c2ccc(-c3cccc4c3oc3ccccc34)cc2)c2cccc3ccccc23)c1 ; c1cc(-c2ccc3c(c2)-c2cccc4cccc(c24)S3)cc(N(c2ccc(-c3cccc4c3oc3ccccc34)cc2)c2cccc3ccccc23)c1]

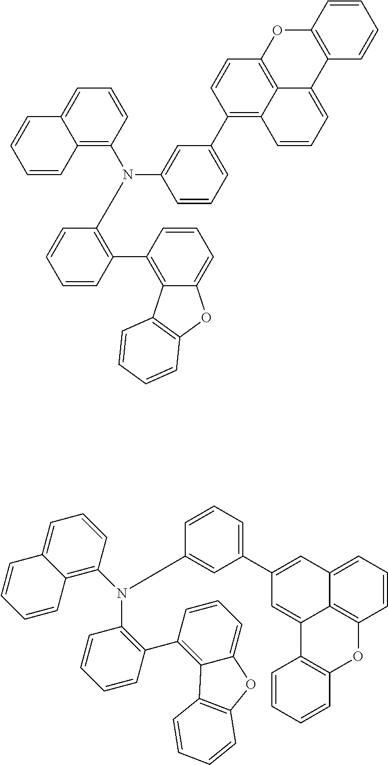 [SMILES: c1cc(-c2ccc3c4c(cccc24)-c2ccccc2O3)cc(N(c2ccccc2-c2cccc3oc4ccccc4c23)c2cccc3ccccc23)c1 ; c1cc(-c2cc3c4c(cccc4c2)Oc2ccccc2-3)cc(N(c2ccccc2-c2cccc3oc4ccccc4c23)c2cccc3ccccc23)c1]

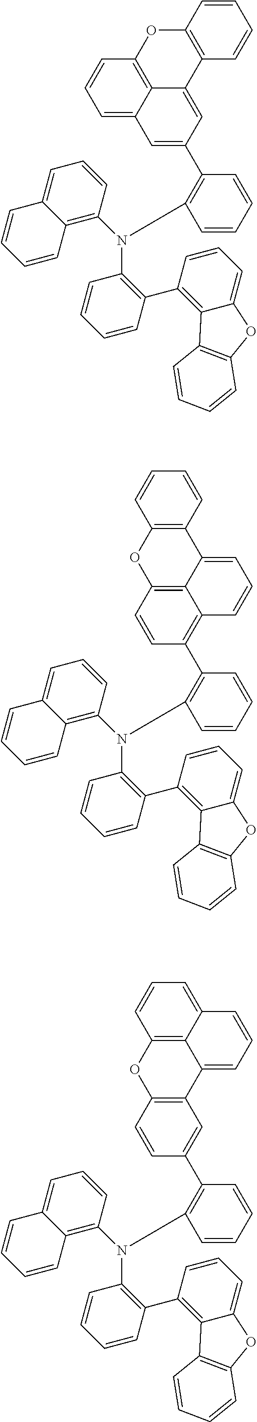 [SMILES: c1ccc2c(c1)Oc1cccc3cc(-c4ccccc4N(c4ccccc4-c4cccc5oc6ccccc6c45)c4cccc5ccccc45)cc-2c13 ; c1ccc2c(c1)Oc1ccc(-c3ccccc3N(c3ccccc3-c3cccc4oc5ccccc5c34)c3cccc4ccccc34)c3cccc-2c13 ; c1ccc(N(c2ccccc2-c2cccc3oc4ccccc4c23)c2cccc3ccccc23)c(-c2ccc3c(c2)-c2cccc4cccc(c24)O3)c1]

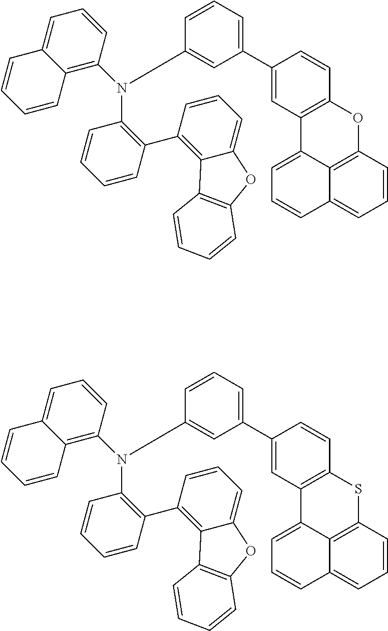 [SMILES: c1cc(-c2ccc3c(c2)-c2cccc4cccc(c24)O3)cc(N(c2ccccc2-c2cccc3oc4ccccc4c23)c2cccc3ccccc23)c1 ; c1cc(-c2ccc3c(c2)-c2cccc4cccc(c24)S3)cc(N(c2ccccc2-c2cccc3oc4ccccc4c23)c2cccc3ccccc23)c1]

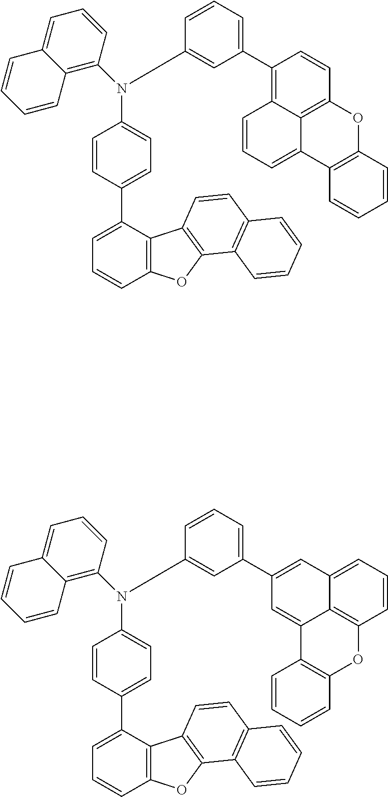 [SMILES: c1cc(-c2ccc3c4c(cccc24)-c2ccccc2O3)cc(N(c2ccc(-c3cccc4oc5c6ccccc6ccc5c34)cc2)c2cccc3ccccc23)c1 ; c1cc(-c2cc3c4c(cccc4c2)Oc2ccccc2-3)cc(N(c2ccc(-c3cccc4oc5c6ccccc6ccc5c34)cc2)c2cccc3ccccc23)c1]

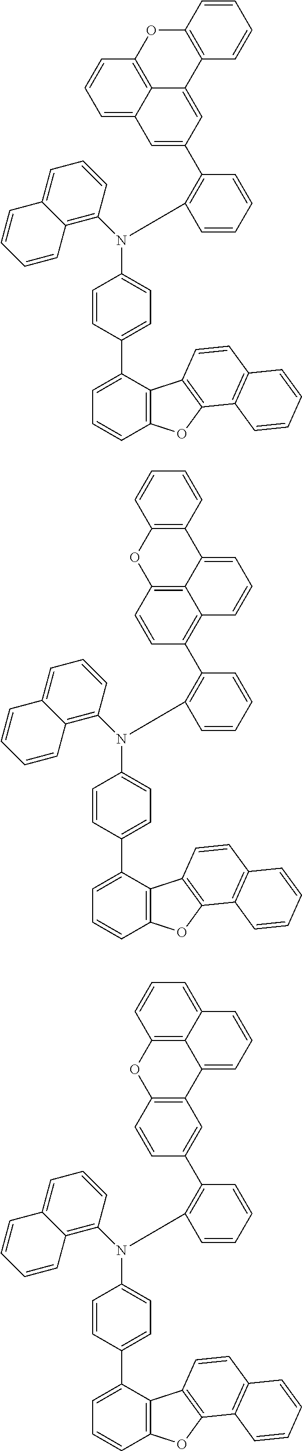

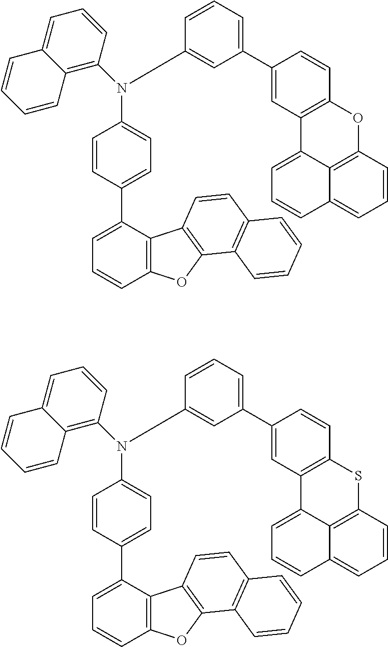 [SMILES: c1cc(-c2ccc3c(c2)-c2cccc4cccc(c24)O3)cc(N(c2ccc(-c3cccc4oc5c6ccccc6ccc5c34)cc2)c2cccc3ccccc23)c1 ; c1cc(-c2ccc3c(c2)-c2cccc4cccc(c24)S3)cc(N(c2ccc(-c3cccc4oc5c6ccccc6ccc5c34)cc2)c2cccc3ccccc23)c1]

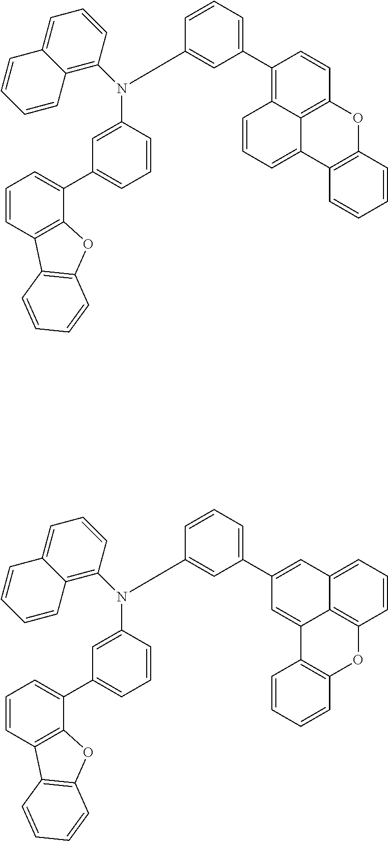 [SMILES: c1cc(-c2ccc3c4c(cccc24)-c2ccccc2O3)cc(N(c2cccc(-c3cccc4c3oc3ccccc34)c2)c2cccc3ccccc23)c1 ; c1cc(-c2cc3c4c(cccc4c2)Oc2ccccc2-3)cc(N(c2cccc(-c3cccc4c3oc3ccccc34)c2)c2cccc3ccccc23)c1]

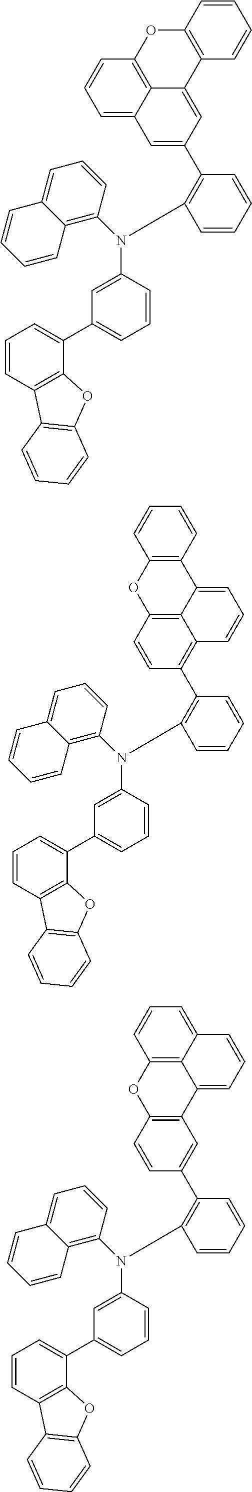 [SMILES: c1cc(-c2cccc3c2oc2ccccc23)cc(N(c2ccccc2-c2cc3c4c(cccc4c2)Oc2ccccc2-3)c2cccc3ccccc23)c1 ; c1cc(-c2cccc3c2oc2ccccc23)cc(N(c2ccccc2-c2ccc3c4c(cccc24)-c2ccccc2O3)c2cccc3ccccc23)c1 ; c1cc(-c2cccc3c2oc2ccccc23)cc(N(c2ccccc2-c2ccc3c(c2)-c2cccc4cccc(c24)O3)c2cccc3ccccc23)c1]

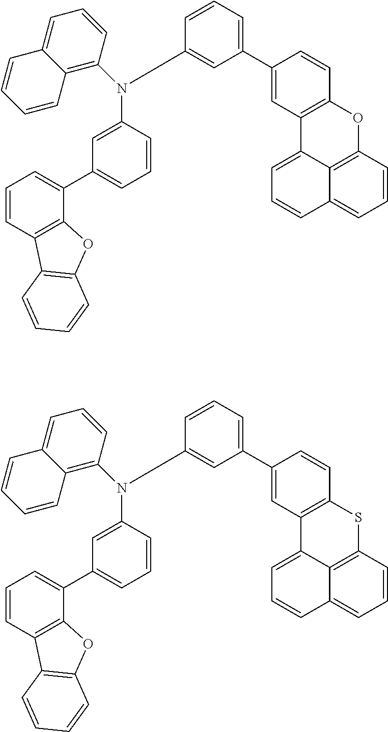 [SMILES: c1cc(-c2ccc3c(c2)-c2cccc4cccc(c24)O3)cc(N(c2cccc(-c3cccc4c3oc3ccccc34)c2)c2cccc3ccccc23)c1 ; c1cc(-c2ccc3c(c2)-c2cccc4cccc(c24)S3)cc(N(c2cccc(-c3cccc4c3oc3ccccc34)c2)c2cccc3ccccc23)c1]

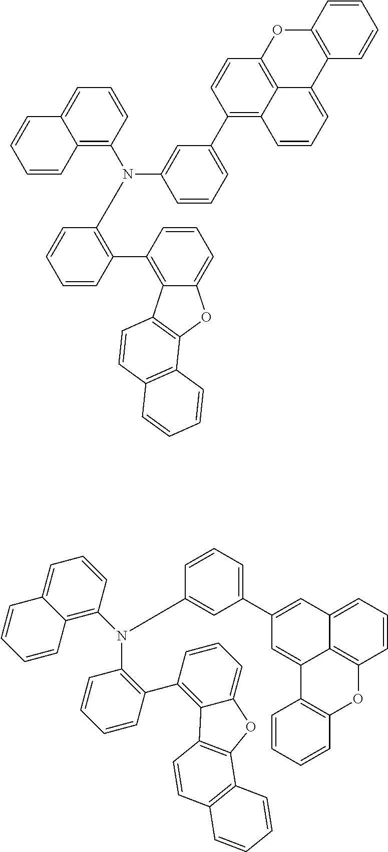 [SMILES: c1cc(-c2ccc3c4c(cccc24)-c2ccccc2O3)cc(N(c2ccccc2-c2cccc3oc4c5ccccc5ccc4c23)c2cccc3ccccc23)c1 ; c1cc(-c2cc3c4c(cccc4c2)Oc2ccccc2-3)cc(N(c2ccccc2-c2cccc3oc4c5ccccc5ccc4c23)c2cccc3ccccc23)c1]

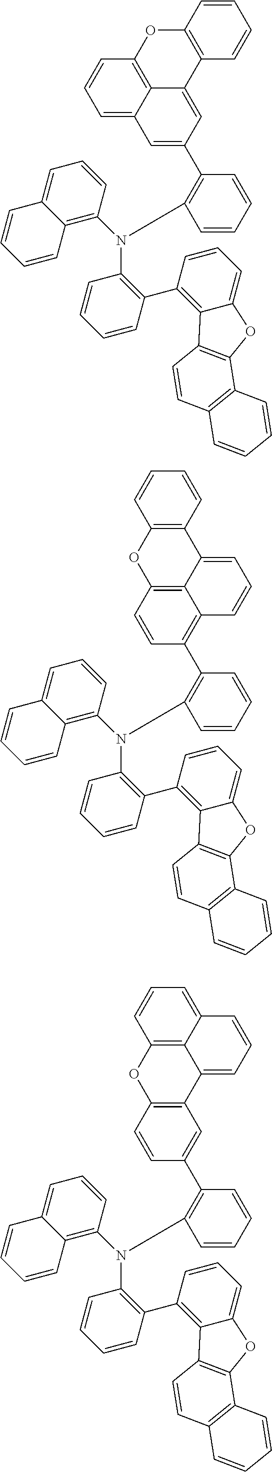

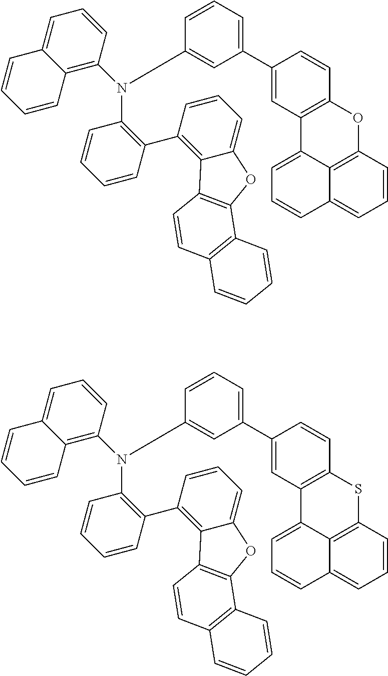 [SMILES: c1cc(-c2ccc3c(c2)-c2cccc4cccc(c24)O3)cc(N(c2ccccc2-c2cccc3oc4c5ccccc5ccc4c23)c2cccc3ccccc23)c1 ; c1cc(-c2ccc3c(c2)-c2cccc4cccc(c24)S3)cc(N(c2ccccc2-c2cccc3oc4c5ccccc5ccc4c23)c2cccc3ccccc23)c1]

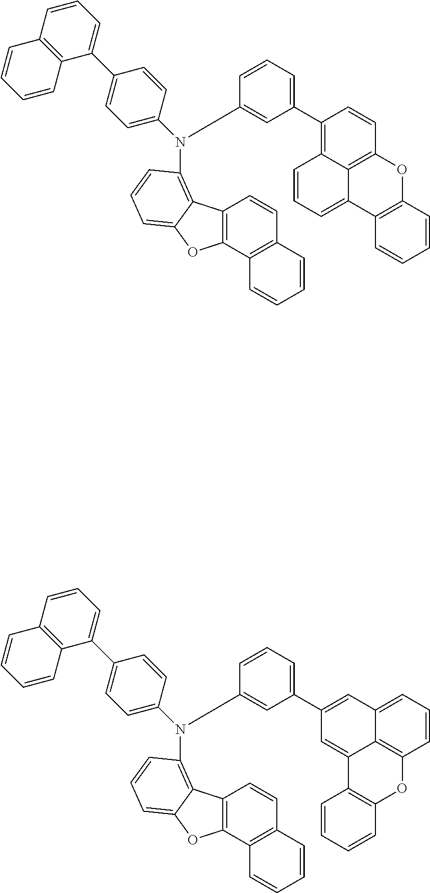 [SMILES: c1cc(-c2ccc3c4c(cccc24)-c2ccccc2O3)cc(N(c2ccc(-c3cccc4ccccc34)cc2)c2cccc3oc4c5ccccc5ccc4c23)c1 ; c1cc(-c2cc3c4c(cccc4c2)Oc2ccccc2-3)cc(N(c2ccc(-c3cccc4ccccc34)cc2)c2cccc3oc4c5ccccc5ccc4c23)c1]

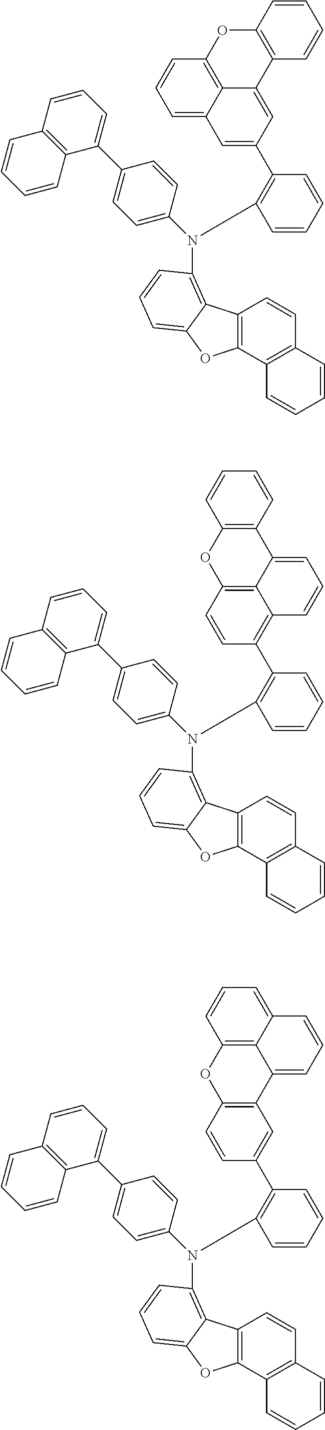 [SMILES: c1ccc2c(c1)Oc1cccc3cc(-c4ccccc4N(c4ccc(-c5cccc6ccccc56)cc4)c4cccc5oc6c7ccccc7ccc6c45)cc-2c13 ; c1ccc2c(c1)Oc1ccc(-c3ccccc3N(c3ccc(-c4cccc5ccccc45)cc3)c3cccc4oc5c6ccccc6ccc5c34)c3cccc-2c13 ; c1ccc(N(c2ccc(-c3cccc4ccccc34)cc2)c2cccc3oc4c5ccccc5ccc4c23)c(-c2ccc3c(c2)-c2cccc4cccc(c24)O3)c1]

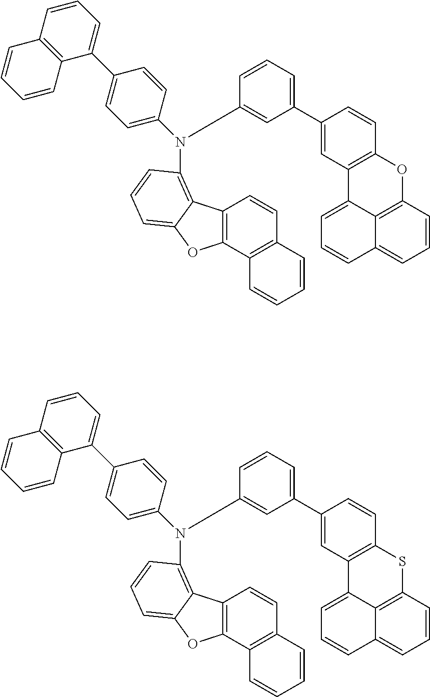 [SMILES: c1cc(-c2ccc3c(c2)-c2cccc4cccc(c24)O3)cc(N(c2ccc(-c3cccc4ccccc34)cc2)c2cccc3oc4c5ccccc5ccc4c23)c1 ; c1cc(-c2ccc3c(c2)-c2cccc4cccc(c24)S3)cc(N(c2ccc(-c3cccc4ccccc34)cc2)c2cccc3oc4c5ccccc5ccc4c23)c1]

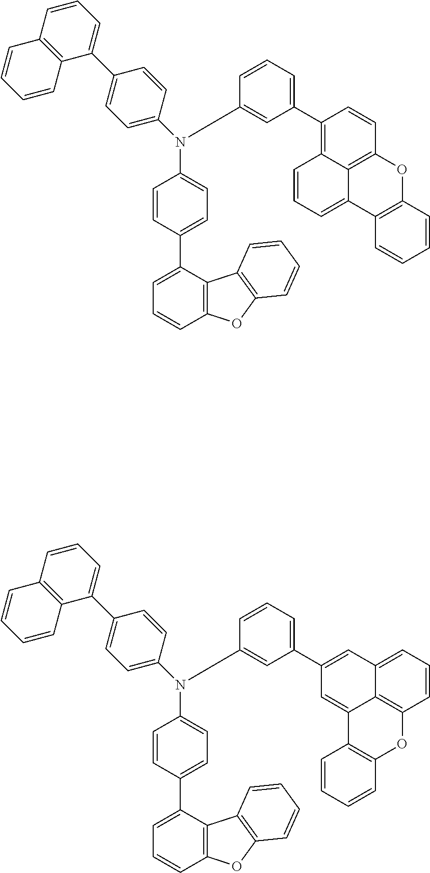 [SMILES: c1cc(-c2ccc3c4c(cccc24)-c2ccccc2O3)cc(N(c2ccc(-c3cccc4ccccc34)cc2)c2ccc(-c3cccc4oc5ccccc5c34)cc2)c1 ; c1cc(-c2cc3c4c(cccc4c2)Oc2ccccc2-3)cc(N(c2ccc(-c3cccc4ccccc34)cc2)c2ccc(-c3cccc4oc5ccccc5c34)cc2)c1]

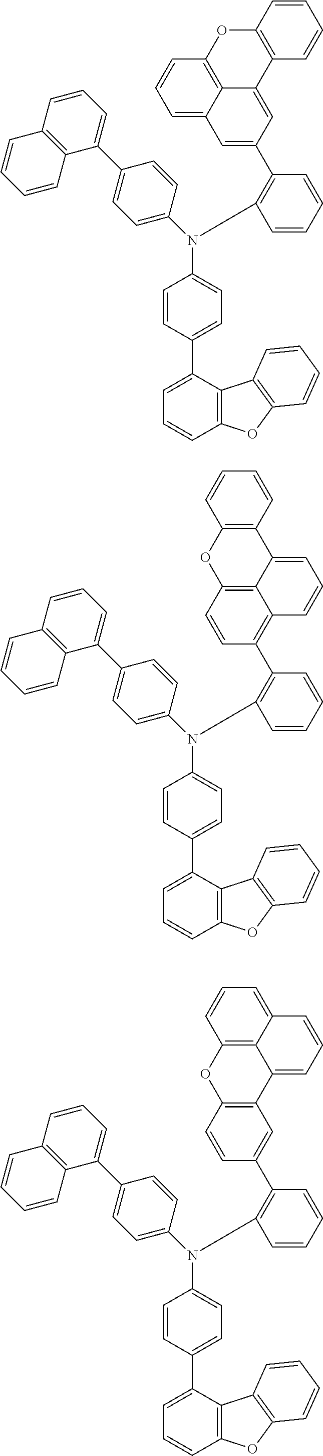

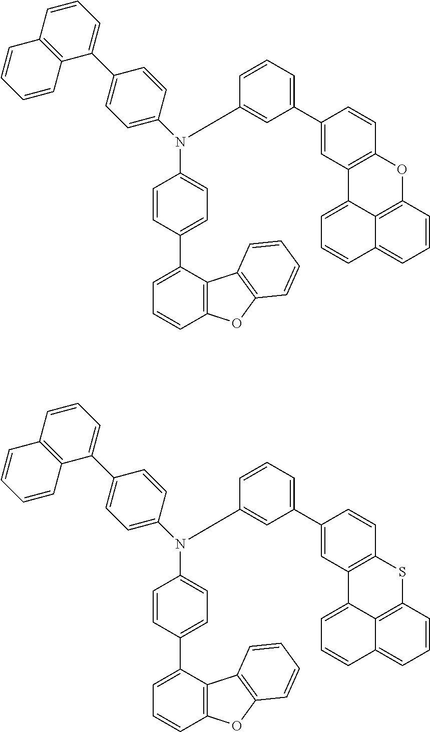 [SMILES: c1cc(-c2ccc3c(c2)-c2cccc4cccc(c24)O3)cc(N(c2ccc(-c3cccc4ccccc34)cc2)c2ccc(-c3cccc4oc5ccccc5c34)cc2)c1 ; c1cc(-c2ccc3c(c2)-c2cccc4cccc(c24)S3)cc(N(c2ccc(-c3cccc4ccccc34)cc2)c2ccc(-c3cccc4oc5ccccc5c34)cc2)c1]

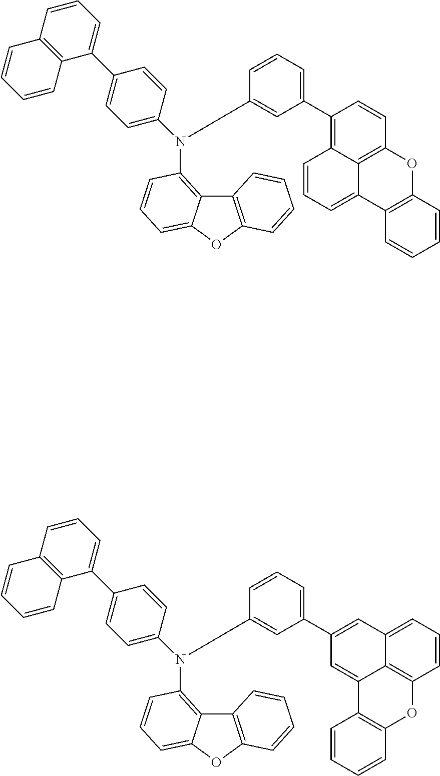 [SMILES: c1cc(-c2ccc3c4c(cccc24)-c2ccccc2O3)cc(N(c2ccc(-c3cccc4ccccc34)cc2)c2cccc3oc4ccccc4c23)c1 ; c1cc(-c2cc3c4c(cccc4c2)Oc2ccccc2-3)cc(N(c2ccc(-c3cccc4ccccc34)cc2)c2cccc3oc4ccccc4c23)c1]

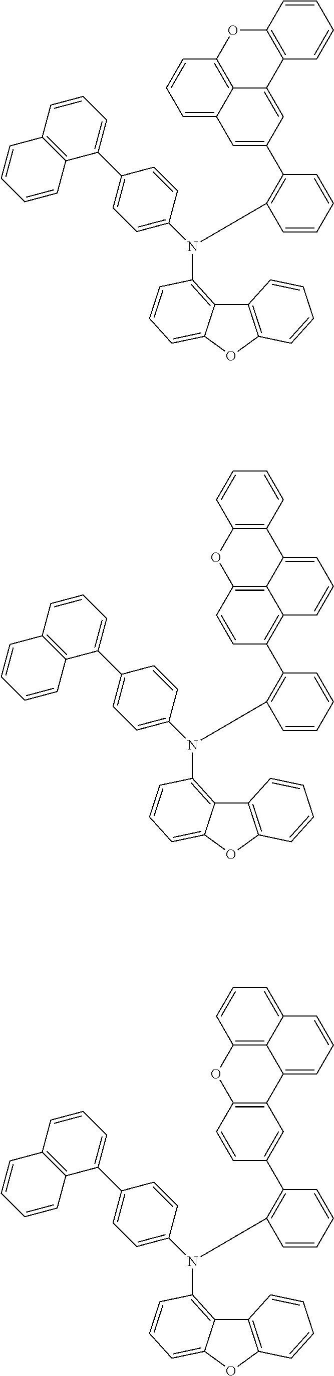 [SMILES: c1ccc2c(c1)Oc1cccc3cc(-c4ccccc4N(c4ccc(-c5cccc6ccccc56)cc4)c4cccc5oc6ccccc6c45)cc-2c13 ; c1ccc2c(c1)Oc1ccc(-c3ccccc3N(c3ccc(-c4cccc5ccccc45)cc3)c3cccc4oc5ccccc5c34)c3cccc-2c13 ; c1ccc(N(c2ccc(-c3cccc4ccccc34)cc2)c2cccc3oc4ccccc4c23)c(-c2ccc3c(c2)-c2cccc4cccc(c24)O3)c1]

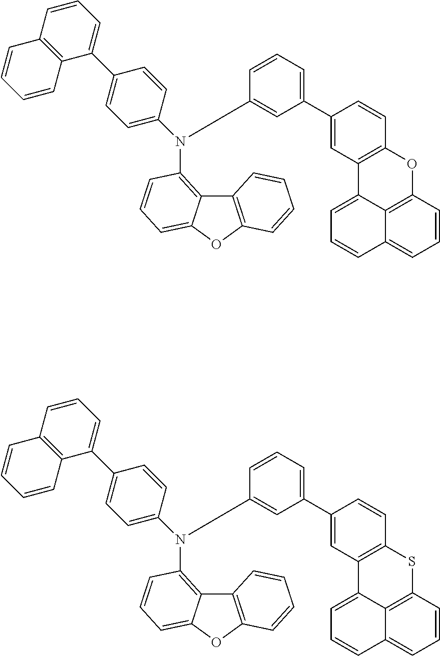 [SMILES: c1cc(-c2ccc3c(c2)-c2cccc4cccc(c24)O3)cc(N(c2ccc(-c3cccc4ccccc34)cc2)c2cccc3oc4ccccc4c23)c1 ; c1cc(-c2ccc3c(c2)-c2cccc4cccc(c24)S3)cc(N(c2ccc(-c3cccc4ccccc34)cc2)c2cccc3oc4ccccc4c23)c1]

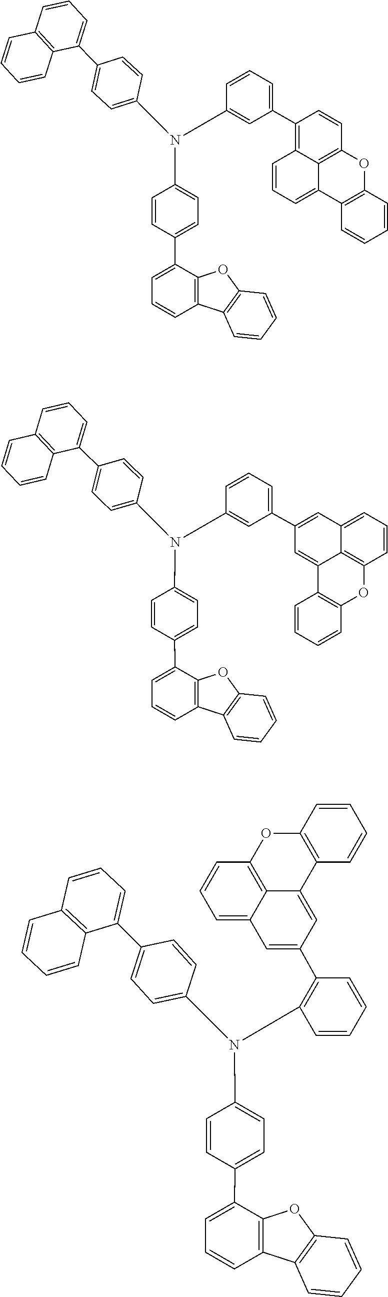 [SMILES: c1cc(-c2ccc3c4c(cccc24)-c2ccccc2O3)cc(N(c2ccc(-c3cccc4ccccc34)cc2)c2ccc(-c3cccc4c3oc3ccccc34)cc2)c1 ; c1cc(-c2cc3c4c(cccc4c2)Oc2ccccc2-3)cc(N(c2ccc(-c3cccc4ccccc34)cc2)c2ccc(-c3cccc4c3oc3ccccc34)cc2)c1 ; c1ccc2c(c1)Oc1cccc3cc(-c4ccccc4N(c4ccc(-c5cccc6ccccc56)cc4)c4ccc(-c5cccc6c5oc5ccccc56)cc4)cc-2c13]

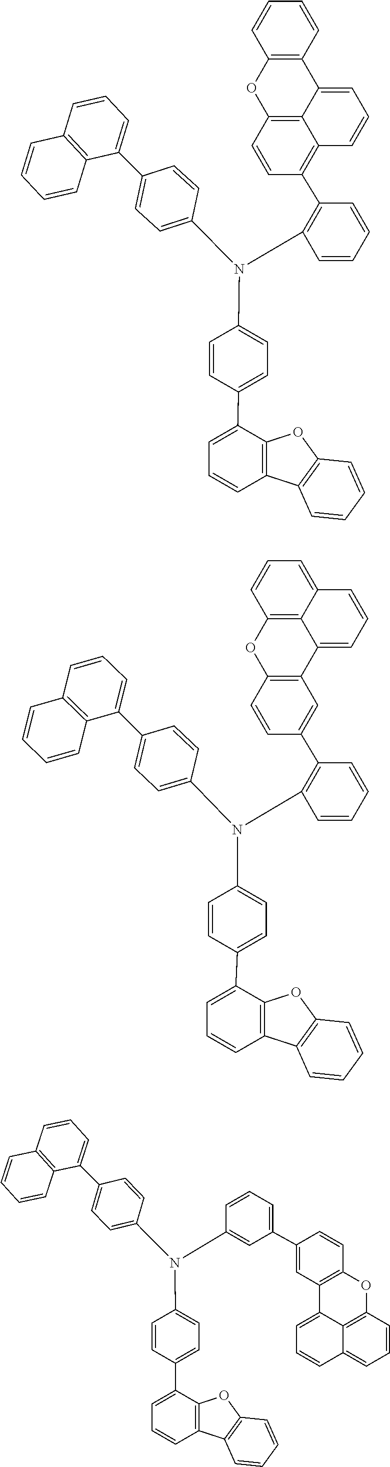

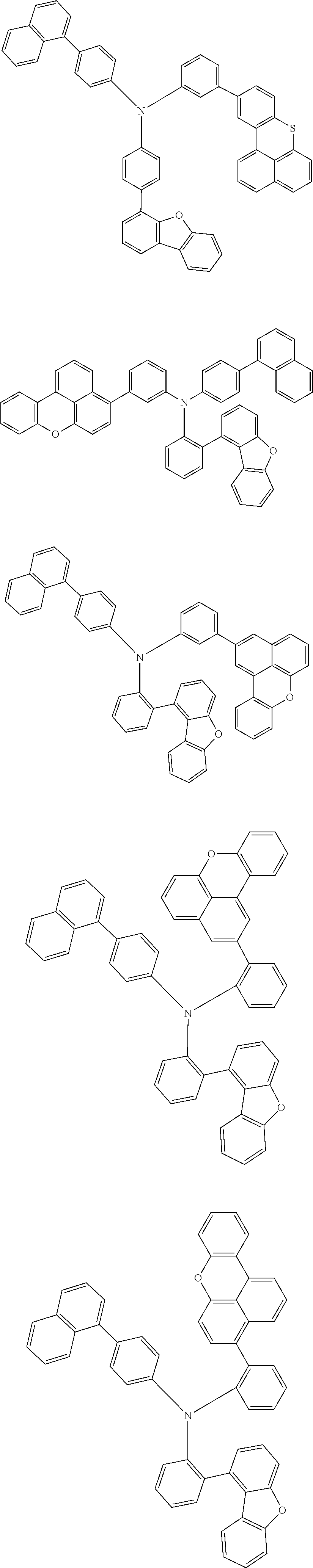 [SMILES: c1cc(-c2ccc3c(c2)-c2cccc4cccc(c24)S3)cc(N(c2ccc(-c3cccc4ccccc34)cc2)c2ccc(-c3cccc4c3oc3ccccc34)cc2)c1 ; c1cc(-c2ccc3c4c(cccc24)-c2ccccc2O3)cc(N(c2ccc(-c3cccc4ccccc34)cc2)c2ccccc2-c2cccc3oc4ccccc4c23)c1 ; c1cc(-c2cc3c4c(cccc4c2)Oc2ccccc2-3)cc(N(c2ccc(-c3cccc4ccccc34)cc2)c2ccccc2-c2cccc3oc4ccccc4c23)c1 ; c1ccc2c(c1)Oc1cccc3cc(-c4ccccc4N(c4ccc(-c5cccc6ccccc56)cc4)c4ccccc4-c4cccc5oc6ccccc6c45)cc-2c13 ; c1ccc2c(c1)Oc1ccc(-c3ccccc3N(c3ccc(-c4cccc5ccccc45)cc3)c3ccccc3-c3cccc4oc5ccccc5c34)c3cccc-2c13]

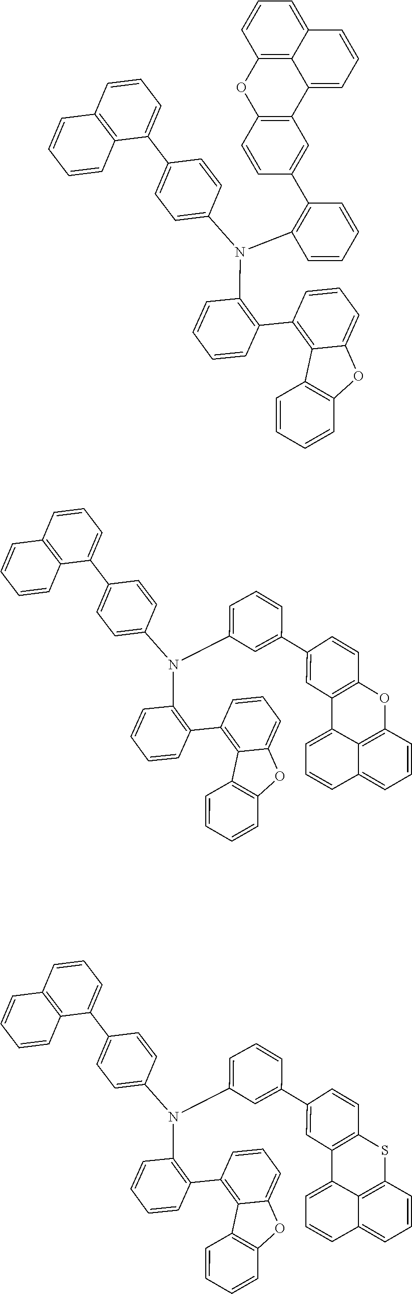 [SMILES: c1ccc(N(c2ccc(-c3cccc4ccccc34)cc2)c2ccccc2-c2cccc3oc4ccccc4c23)c(-c2ccc3c(c2)-c2cccc4cccc(c24)O3)c1 ; c1cc(-c2ccc3c(c2)-c2cccc4cccc(c24)O3)cc(N(c2ccc(-c3cccc4ccccc34)cc2)c2ccccc2-c2cccc3oc4ccccc4c23)c1 ; c1cc(-c2ccc3c(c2)-c2cccc4cccc(c24)S3)cc(N(c2ccc(-c3cccc4ccccc34)cc2)c2ccccc2-c2cccc3oc4ccccc4c23)c1]

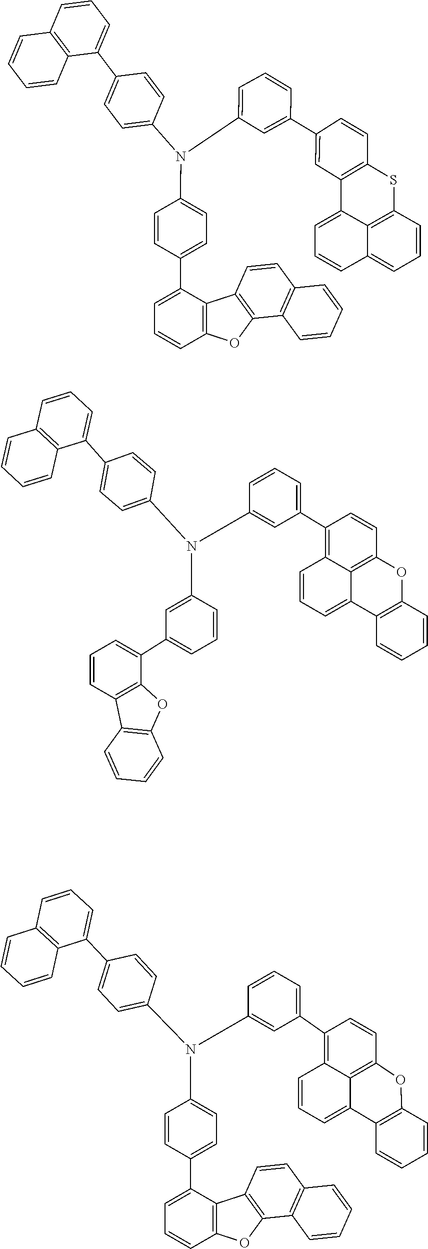

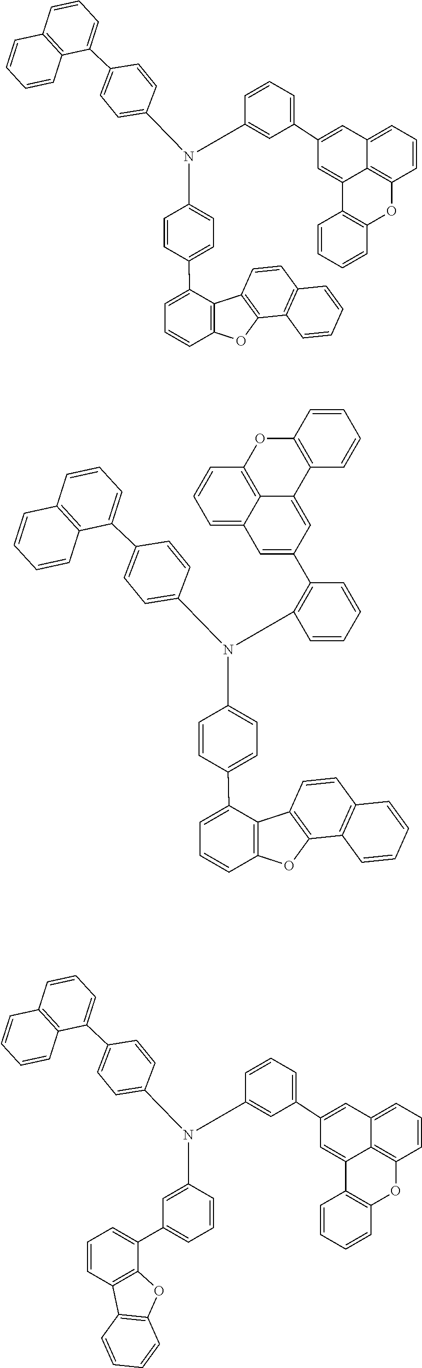 [SMILES: c1cc(-c2cc3c4c(cccc4c2)Oc2ccccc2-3)cc(N(c2ccc(-c3cccc4ccccc34)cc2)c2ccc(-c3cccc4oc5c6ccccc6ccc5c34)cc2)c1 ; c1ccc2c(c1)Oc1cccc3cc(-c4ccccc4N(c4ccc(-c5cccc6ccccc56)cc4)c4ccc(-c5cccc6oc7c8ccccc8ccc7c56)cc4)cc-2c13 ; c1cc(-c2cc3c4c(cccc4c2)Oc2ccccc2-3)cc(N(c2ccc(-c3cccc4ccccc34)cc2)c2cccc(-c3cccc4c3oc3ccccc34)c2)c1]

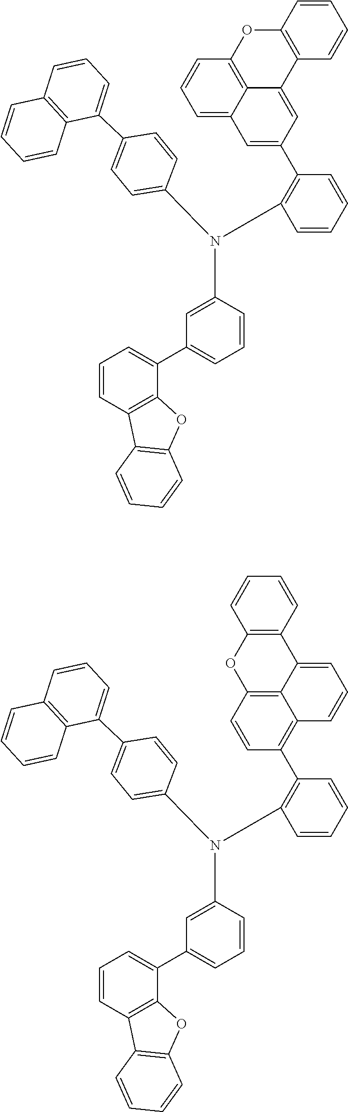 [SMILES: c1cc(-c2cccc3c2oc2ccccc23)cc(N(c2ccc(-c3cccc4ccccc34)cc2)c2ccccc2-c2cc3c4c(cccc4c2)Oc2ccccc2-3)c1 ; c1cc(-c2cccc3c2oc2ccccc23)cc(N(c2ccc(-c3cccc4ccccc34)cc2)c2ccccc2-c2ccc3c4c(cccc24)-c2ccccc2O3)c1]

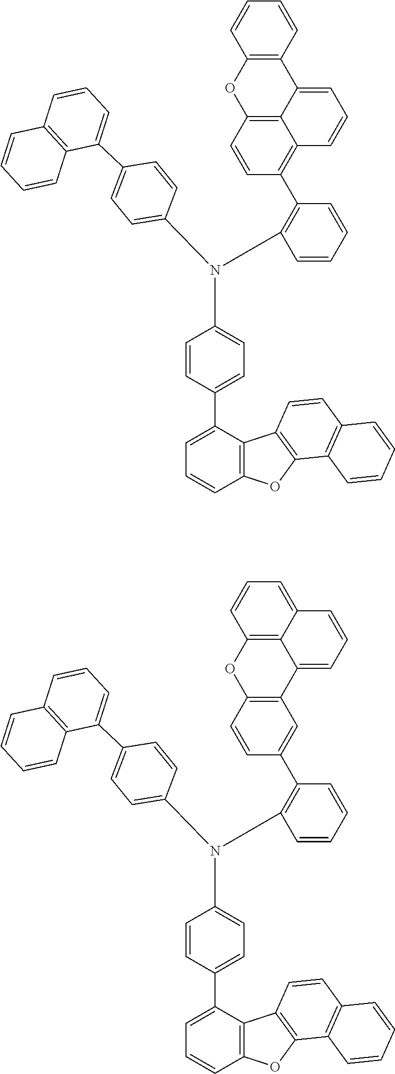 [SMILES: c1ccc2c(c1)Oc1ccc(-c3ccccc3N(c3ccc(-c4cccc5ccccc45)cc3)c3ccc(-c4cccc5oc6c7ccccc7ccc6c45)cc3)c3cccc-2c13 ; c1ccc(N(c2ccc(-c3cccc4ccccc34)cc2)c2ccc(-c3cccc4oc5c6ccccc6ccc5c34)cc2)c(-c2ccc3c(c2)-c2cccc4cccc(c24)O3)c1]

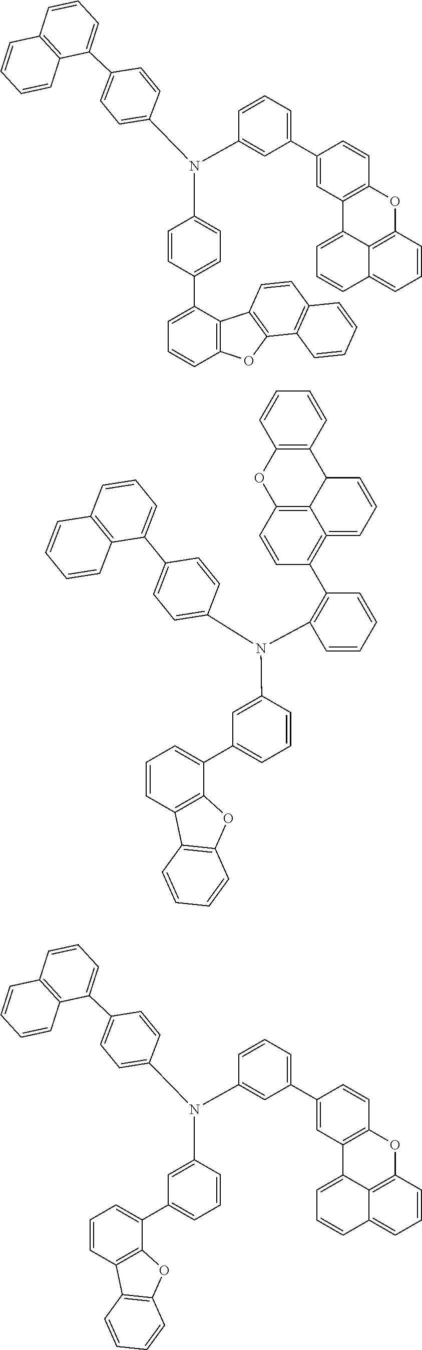

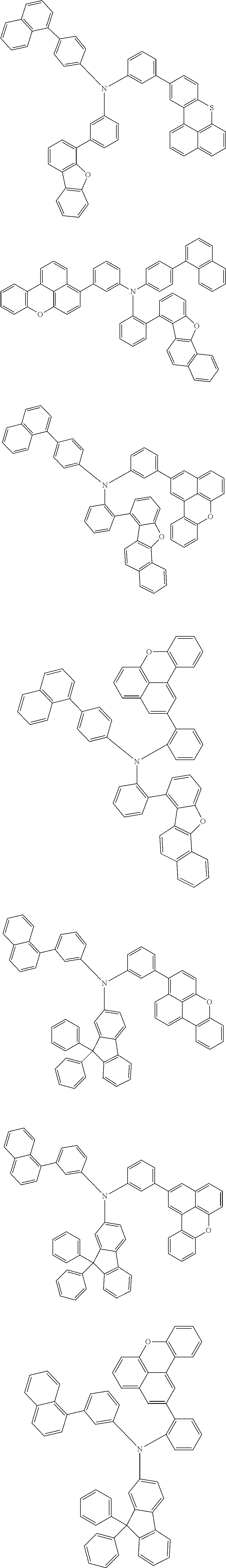

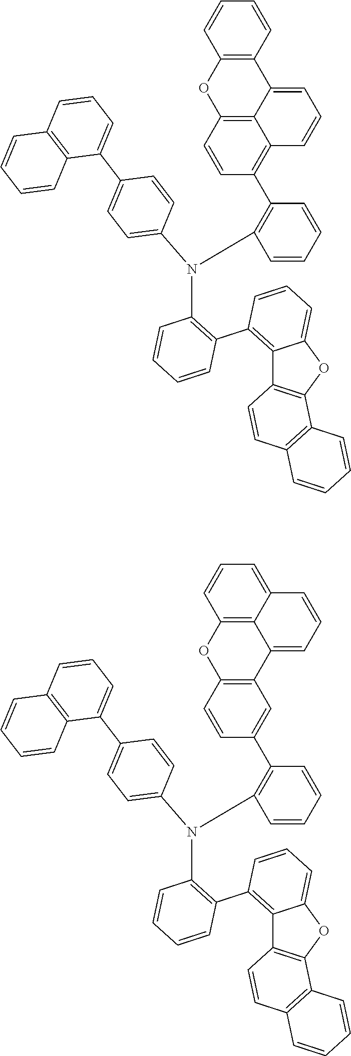 [SMILES: c1ccc2c(c1)Oc1ccc(-c3ccccc3N(c3ccc(-c4cccc5ccccc45)cc3)c3ccccc3-c3cccc4oc5c6ccccc6ccc5c34)c3cccc-2c13 ; c1ccc(N(c2ccc(-c3cccc4ccccc34)cc2)c2ccccc2-c2cccc3oc4c5ccccc5ccc4c23)c(-c2ccc3c(c2)-c2cccc4cccc(c24)O3)c1]

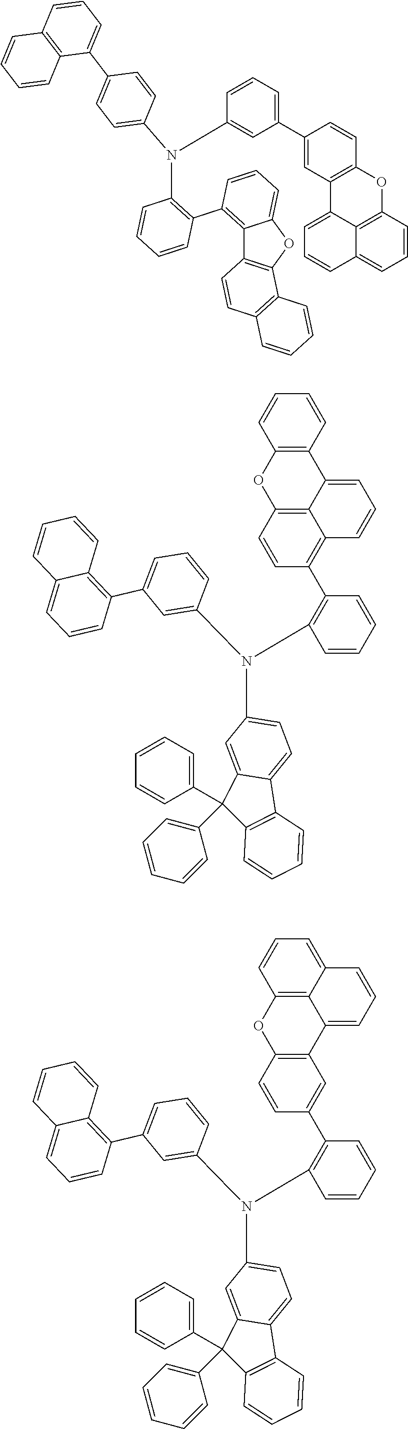

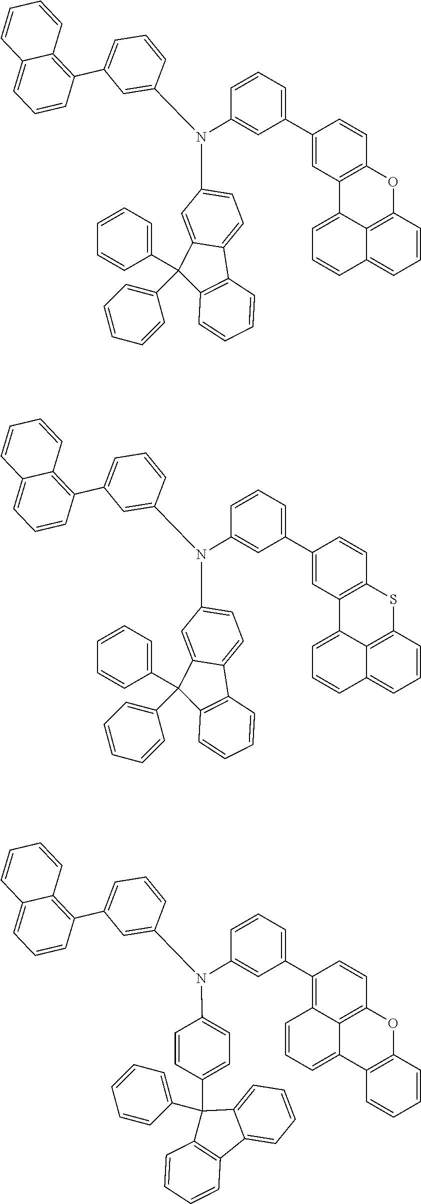 [SMILES: c1ccc(C2(c3ccccc3)c3ccccc3-c3ccc(N(c4cccc(-c5ccc6c(c5)-c5cccc7cccc(c57)O6)c4)c4cccc(-c5cccc6ccccc56)c4)cc32)cc1 ; c1ccc(C2(c3ccccc3)c3ccccc3-c3ccc(N(c4cccc(-c5ccc6c(c5)-c5cccc7cccc(c57)S6)c4)c4cccc(-c5cccc6ccccc56)c4)cc32)cc1 ; c1ccc(C2(c3ccc(N(c4cccc(-c5cccc6ccccc56)c4)c4cccc(-c5ccc6c7c(cccc57)-c5ccccc5O6)c4)cc3)c3ccccc3-c3ccccc32)cc1]

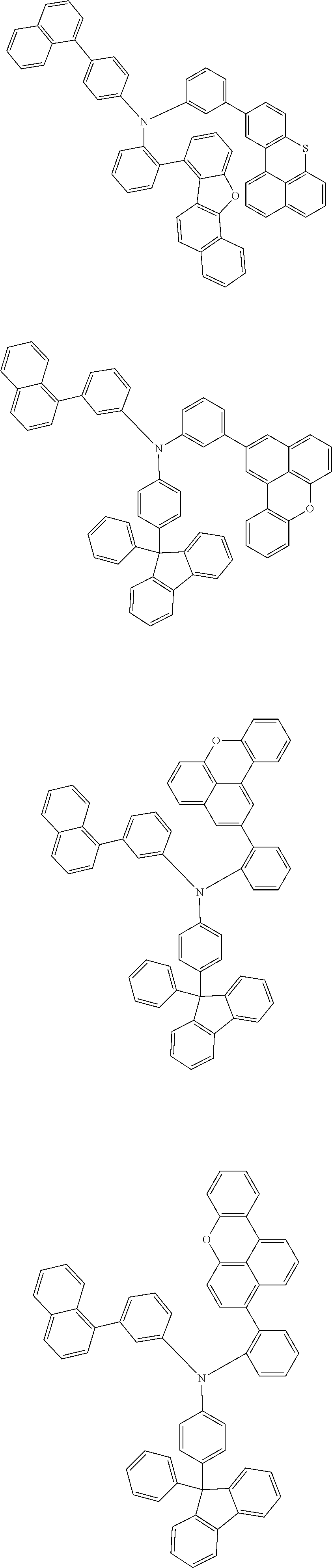 [SMILES: c1cc(-c2ccc3c(c2)-c2cccc4cccc(c24)S3)cc(N(c2ccc(-c3cccc4ccccc34)cc2)c2ccccc2-c2cccc3oc4c5ccccc5ccc4c23)c1 ; c1ccc(C2(c3ccc(N(c4cccc(-c5cc6c7c(cccc7c5)Oc5ccccc5-6)c4)c4cccc(-c5cccc6ccccc56)c4)cc3)c3ccccc3-c3ccccc32)cc1 ; c1ccc(C2(c3ccc(N(c4cccc(-c5cccc6ccccc56)c4)c4ccccc4-c4cc5c6c(cccc6c4)Oc4ccccc4-5)cc3)c3ccccc3-c3ccccc32)cc1 ; c1ccc(C2(c3ccc(N(c4cccc(-c5cccc6ccccc56)c4)c4ccccc4-c4ccc5c6c(cccc46)-c4ccccc4O5)cc3)c3ccccc3-c3ccccc32)cc1]

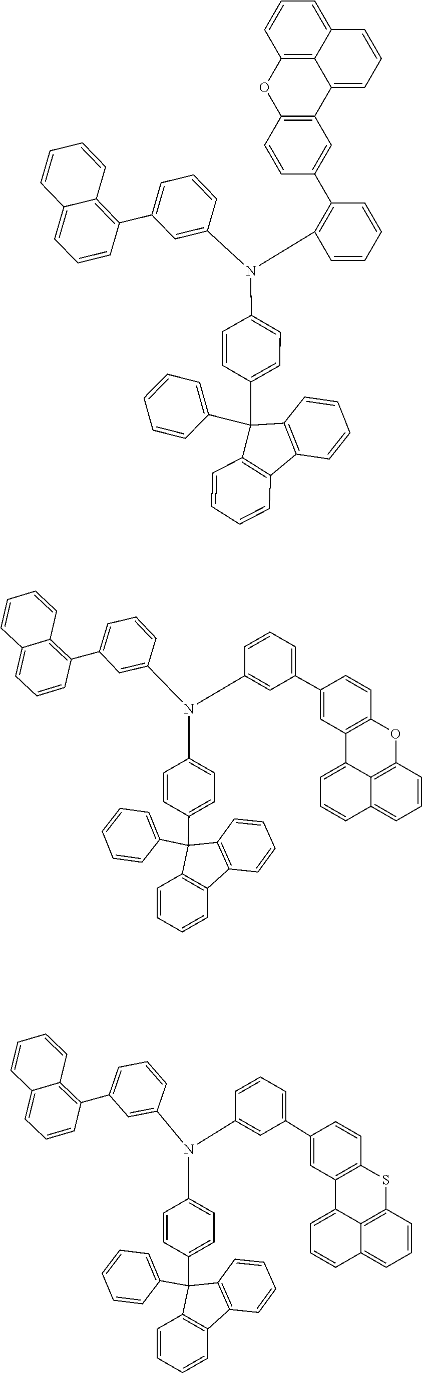 [SMILES: c1ccc(C2(c3ccc(N(c4cccc(-c5cccc6ccccc56)c4)c4ccccc4-c4ccc5c(c4)-c4cccc6cccc(c46)O5)cc3)c3ccccc3-c3ccccc32)cc1 ; c1ccc(C2(c3ccc(N(c4cccc(-c5ccc6c(c5)-c5cccc7cccc(c57)O6)c4)c4cccc(-c5cccc6ccccc56)c4)cc3)c3ccccc3-c3ccccc32)cc1 ; c1ccc(C2(c3ccc(N(c4cccc(-c5ccc6c(c5)-c5cccc7cccc(c57)S6)c4)c4cccc(-c5cccc6ccccc56)c4)cc3)c3ccccc3-c3ccccc32)cc1]

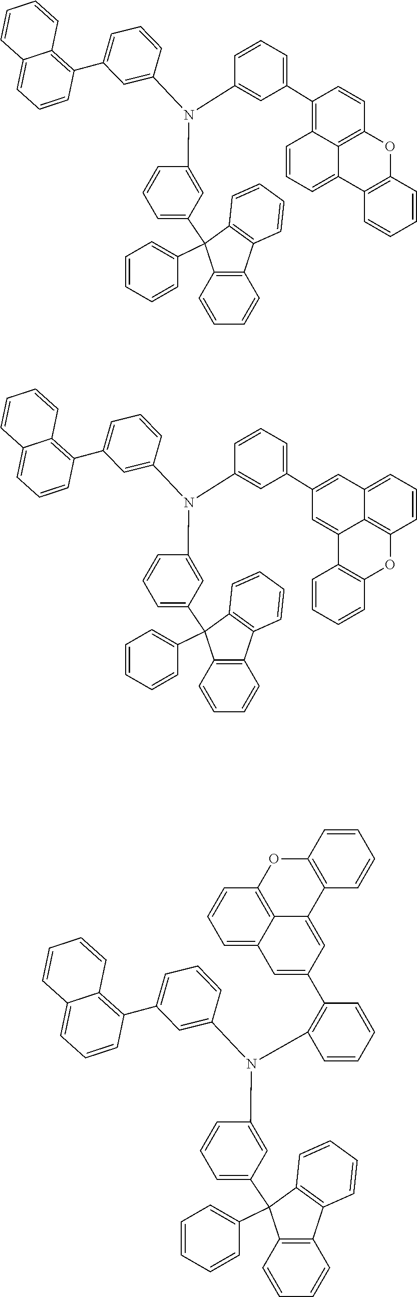 [SMILES: c1ccc(C2(c3cccc(N(c4cccc(-c5cccc6ccccc56)c4)c4cccc(-c5ccc6c7c(cccc57)-c5ccccc5O6)c4)c3)c3ccccc3-c3ccccc32)cc1 ; c1ccc(C2(c3cccc(N(c4cccc(-c5cc6c7c(cccc7c5)Oc5ccccc5-6)c4)c4cccc(-c5cccc6ccccc56)c4)c3)c3ccccc3-c3ccccc32)cc1 ; c1ccc(C2(c3cccc(N(c4cccc(-c5cccc6ccccc56)c4)c4ccccc4-c4cc5c6c(cccc6c4)Oc4ccccc4-5)c3)c3ccccc3-c3ccccc32)cc1]

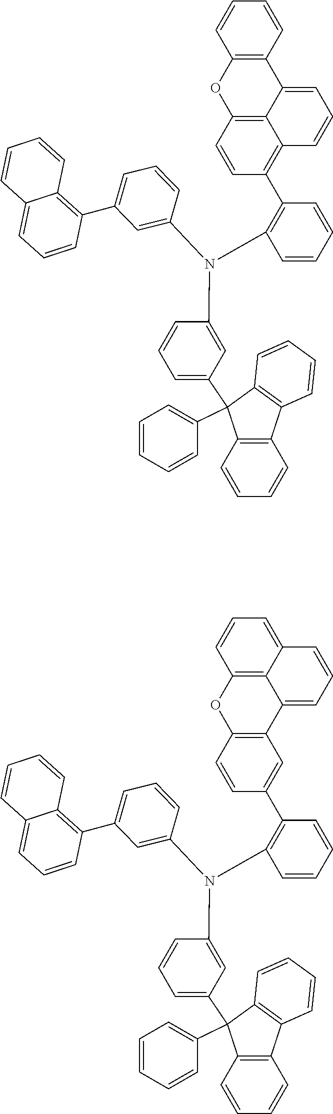 [SMILES: c1ccc(C2(c3cccc(N(c4cccc(-c5cccc6ccccc56)c4)c4ccccc4-c4ccc5c6c(cccc46)-c4ccccc4O5)c3)c3ccccc3-c3ccccc32)cc1 ; c1ccc(C2(c3cccc(N(c4cccc(-c5cccc6ccccc56)c4)c4ccccc4-c4ccc5c(c4)-c4cccc6cccc(c46)O5)c3)c3ccccc3-c3ccccc32)cc1]

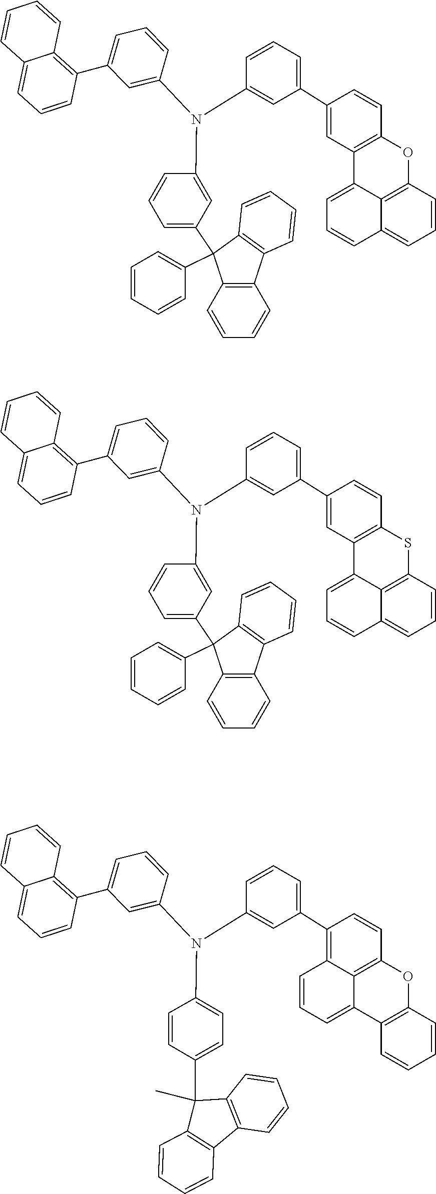 [SMILES: c1ccc(C2(c3cccc(N(c4cccc(-c5ccc6c(c5)-c5cccc7cccc(c57)O6)c4)c4cccc(-c5cccc6ccccc56)c4)c3)c3ccccc3-c3ccccc32)cc1 ; c1ccc(C2(c3cccc(N(c4cccc(-c5ccc6c(c5)-c5cccc7cccc(c57)S6)c4)c4cccc(-c5cccc6ccccc56)c4)c3)c3ccccc3-c3ccccc32)cc1 ; CC1(c2ccc(N(c3cccc(-c4cccc5ccccc45)c3)c3cccc(-c4ccc5c6c(cccc46)-c4ccccc4O5)c3)cc2)c2ccccc2-c2ccccc21]

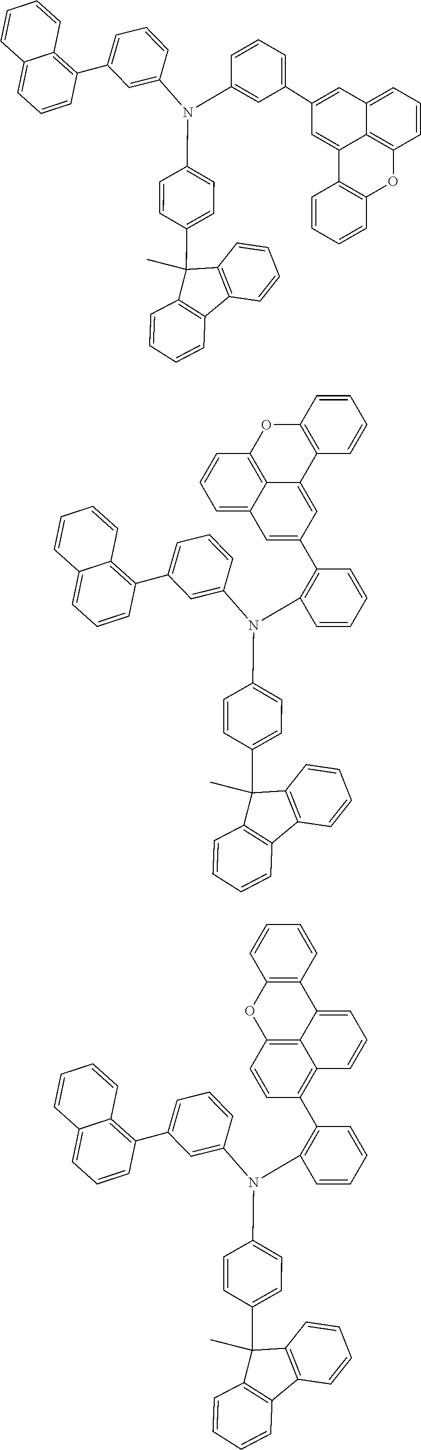

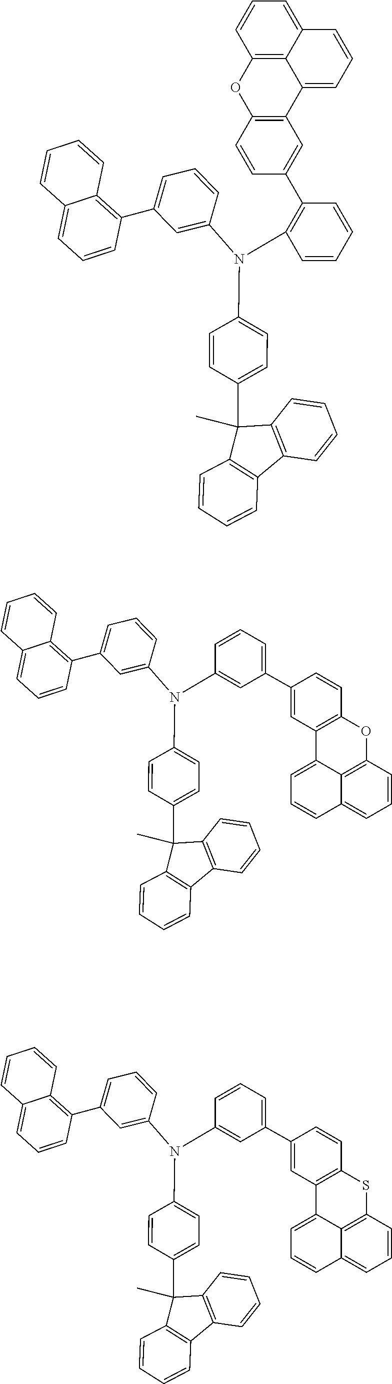 [SMILES: CC1(c2ccc(N(c3cccc(-c4cccc5ccccc45)c3)c3ccccc3-c3ccc4c(c3)-c3cccc5cccc(c35)O4)cc2)c2ccccc2-c2ccccc21 ; CC1(c2ccc(N(c3cccc(-c4ccc5c(c4)-c4cccc6cccc(c46)O5)c3)c3cccc(-c4cccc5ccccc45)c3)cc2)c2ccccc2-c2ccccc21 ; CC1(c2ccc(N(c3cccc(-c4ccc5c(c4)-c4cccc6cccc(c46)S5)c3)c3cccc(-c4cccc5ccccc45)c3)cc2)c2ccccc2-c2ccccc21]

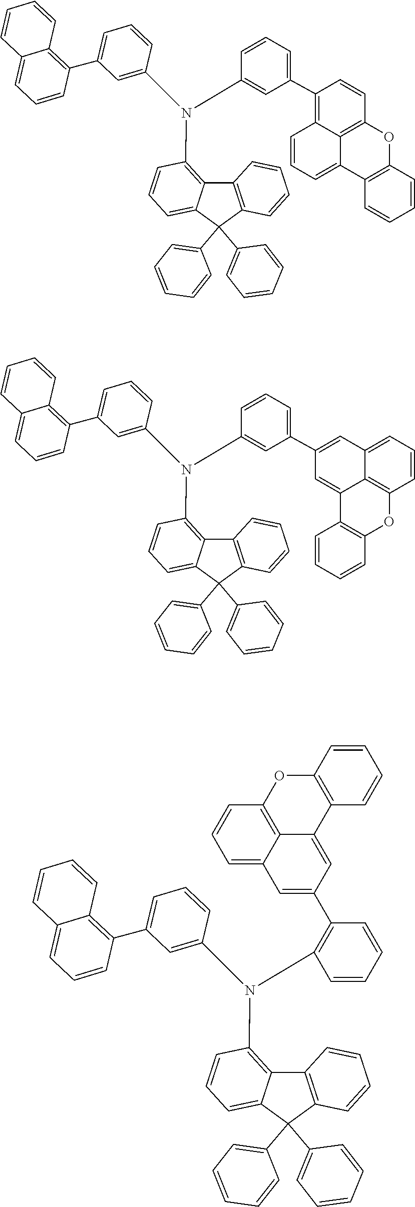 [SMILES: c1ccc(C2(c3ccccc3)c3ccccc3-c3c(N(c4cccc(-c5cccc6ccccc56)c4)c4cccc(-c5ccc6c7c(cccc57)-c5ccccc5O6)c4)cccc32)cc1 ; c1ccc(C2(c3ccccc3)c3ccccc3-c3c(N(c4cccc(-c5cc6c7c(cccc7c5)Oc5ccccc5-6)c4)c4cccc(-c5cccc6ccccc56)c4)cccc32)cc1 ; c1ccc(C2(c3ccccc3)c3ccccc3-c3c(N(c4cccc(-c5cccc6ccccc56)c4)c4ccccc4-c4cc5c6c(cccc6c4)Oc4ccccc4-5)cccc32)cc1]

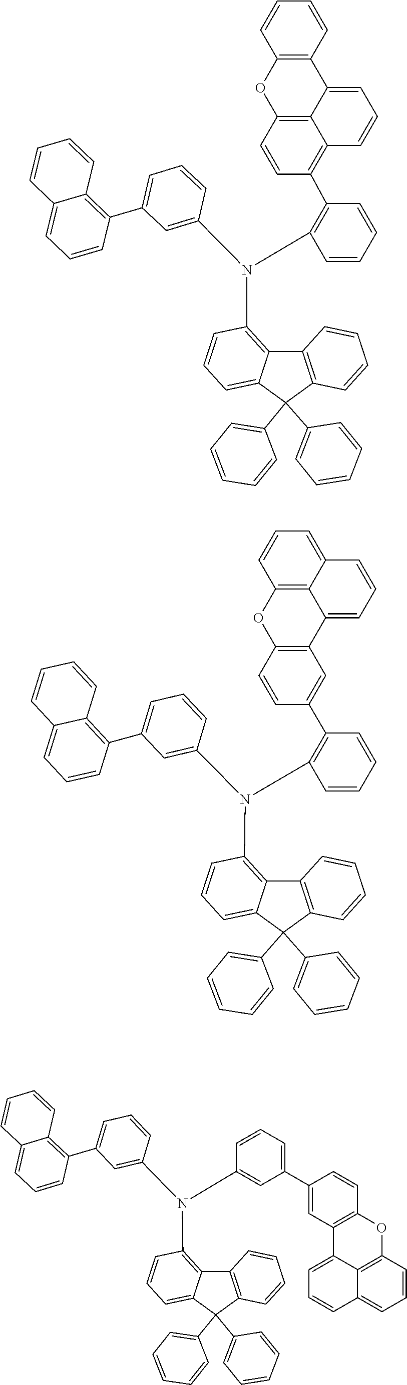 [SMILES: c1ccc(C2(c3ccccc3)c3ccccc3-c3c(N(c4cccc(-c5cccc6ccccc56)c4)c4ccccc4-c4ccc5c6c(cccc46)-c4ccccc4O5)cccc32)cc1 ; c1ccc(C2(c3ccccc3)c3ccccc3-c3c(N(c4cccc(-c5cccc6ccccc56)c4)c4ccccc4-c4ccc5c(c4)-c4cccc6cccc(c46)O5)cccc32)cc1 ; c1ccc(C2(c3ccccc3)c3ccccc3-c3c(N(c4cccc(-c5ccc6c(c5)-c5cccc7cccc(c57)O6)c4)c4cccc(-c5cccc6ccccc56)c4)cccc32)cc1]

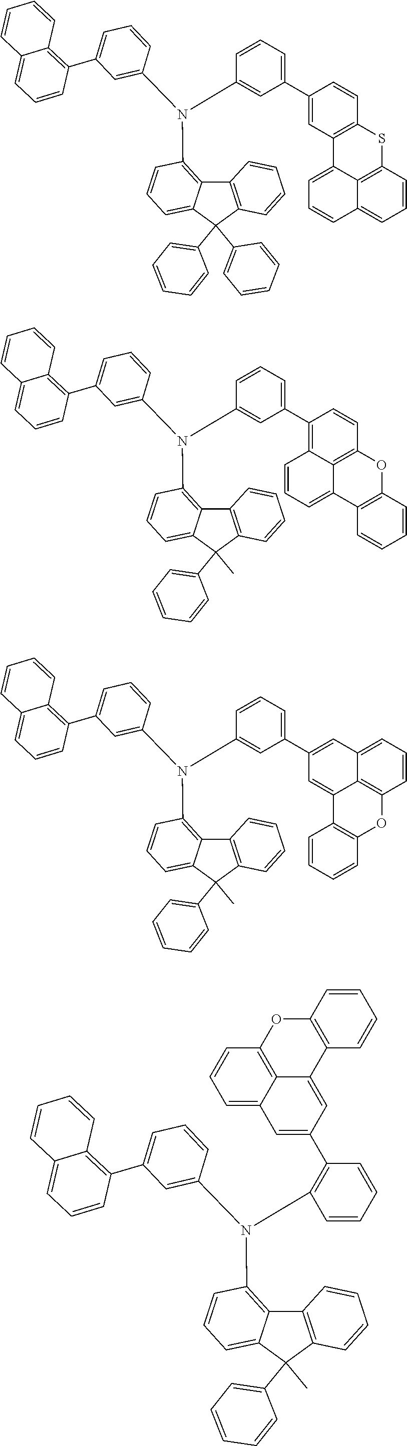 [SMILES: c1ccc(C2(c3ccccc3)c3ccccc3-c3c(N(c4cccc(-c5ccc6c(c5)-c5cccc7cccc(c57)S6)c4)c4cccc(-c5cccc6ccccc56)c4)cccc32)cc1 ; CC1(c2ccccc2)c2ccccc2-c2c(N(c3cccc(-c4cccc5ccccc45)c3)c3cccc(-c4ccc5c6c(cccc46)-c4ccccc4O5)c3)cccc21 ; CC1(c2ccccc2)c2ccccc2-c2c(N(c3cccc(-c4cc5c6c(cccc6c4)Oc4ccccc4-5)c3)c3cccc(-c4cccc5ccccc45)c3)cccc21 ; CC1(c2ccccc2)c2ccccc2-c2c(N(c3cccc(-c4cccc5ccccc45)c3)c3ccccc3-c3cc4c5c(cccc5c3)Oc3ccccc3-4)cccc21]

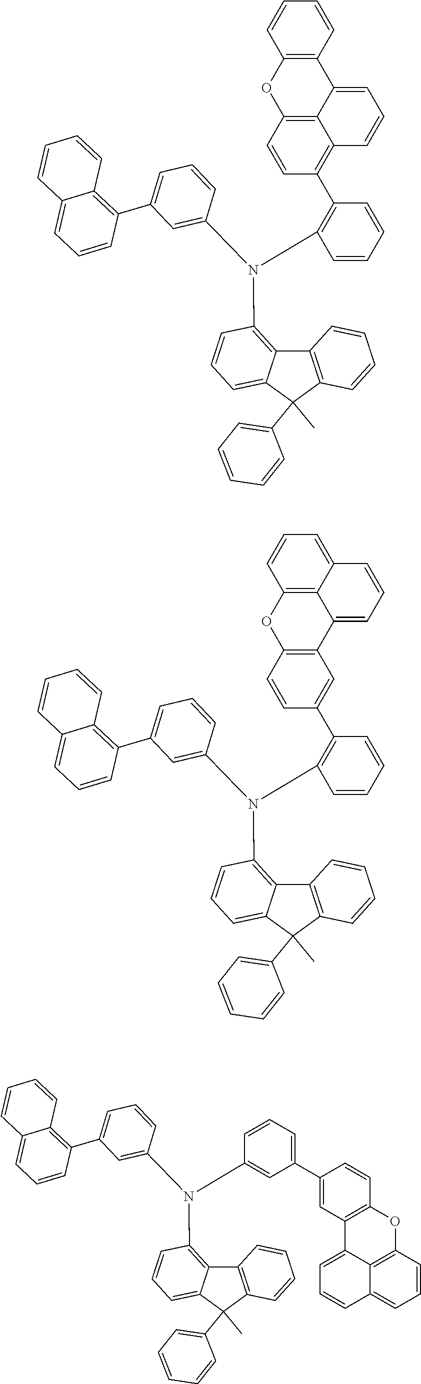 [SMILES: CC1(c2ccccc2)c2ccccc2-c2c(N(c3cccc(-c4cccc5ccccc45)c3)c3ccccc3-c3ccc4c5c(cccc35)-c3ccccc3O4)cccc21 ; CC1(c2ccccc2)c2ccccc2-c2c(N(c3cccc(-c4cccc5ccccc45)c3)c3ccccc3-c3ccc4c(c3)-c3cccc5cccc(c35)O4)cccc21 ; CC1(c2ccccc2)c2ccccc2-c2c(N(c3cccc(-c4ccc5c(c4)-c4cccc6cccc(c46)O5)c3)c3cccc(-c4cccc5ccccc45)c3)cccc21]

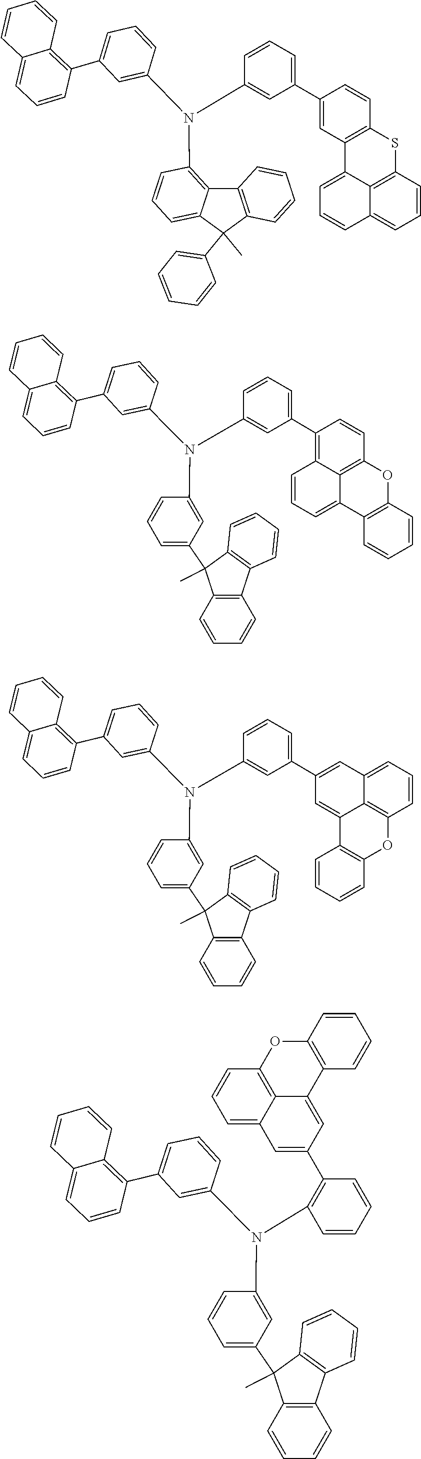 [SMILES: CC1(c2ccccc2)c2ccccc2-c2c(N(c3cccc(-c4ccc5c(c4)-c4cccc6cccc(c46)S5)c3)c3cccc(-c4cccc5ccccc45)c3)cccc21 ; CC1(c2cccc(N(c3cccc(-c4cccc5ccccc45)c3)c3cccc(-c4ccc5c6c(cccc46)-c4ccccc4O5)c3)c2)c2ccccc2-c2ccccc21 ; CC1(c2cccc(N(c3cccc(-c4cc5c6c(cccc6c4)Oc4ccccc4-5)c3)c3cccc(-c4cccc5ccccc45)c3)c2)c2ccccc2-c2ccccc21 ; CC1(c2cccc(N(c3cccc(-c4cccc5ccccc45)c3)c3ccccc3-c3cc4c5c(cccc5c3)Oc3ccccc3-4)c2)c2ccccc2-c2ccccc21]

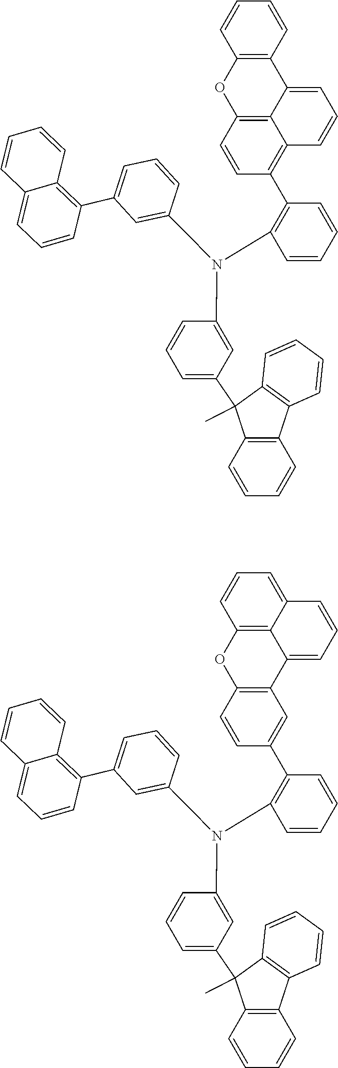 [SMILES: CC1(c2cccc(N(c3cccc(-c4cccc5ccccc45)c3)c3ccccc3-c3ccc4c5c(cccc35)-c3ccccc3O4)c2)c2ccccc2-c2ccccc21 ; CC1(c2cccc(N(c3cccc(-c4cccc5ccccc45)c3)c3ccccc3-c3ccc4c(c3)-c3cccc5cccc(c35)O4)c2)c2ccccc2-c2ccccc21]

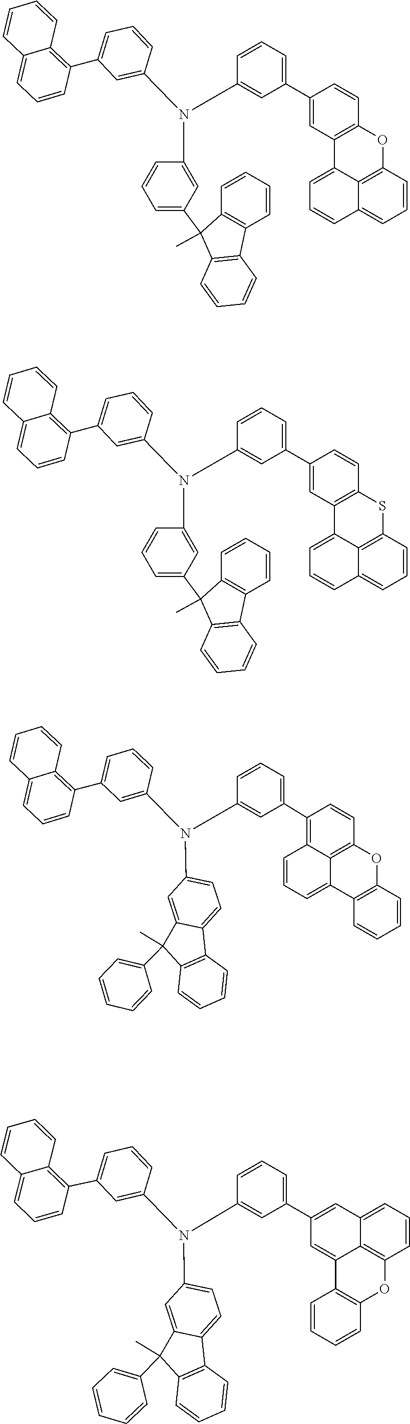 [SMILES: CC1(c2cccc(N(c3cccc(-c4ccc5c(c4)-c4cccc6cccc(c46)O5)c3)c3cccc(-c4cccc5ccccc45)c3)c2)c2ccccc2-c2ccccc21 ; CC1(c2cccc(N(c3cccc(-c4ccc5c(c4)-c4cccc6cccc(c46)S5)c3)c3cccc(-c4cccc5ccccc45)c3)c2)c2ccccc2-c2ccccc21 ; CC1(c2ccccc2)c2ccccc2-c2ccc(N(c3cccc(-c4cccc5ccccc45)c3)c3cccc(-c4ccc5c6c(cccc46)-c4ccccc4O5)c3)cc21 ; CC1(c2ccccc2)c2ccccc2-c2ccc(N(c3cccc(-c4cc5c6c(cccc6c4)Oc4ccccc4-5)c3)c3cccc(-c4cccc5ccccc45)c3)cc21]

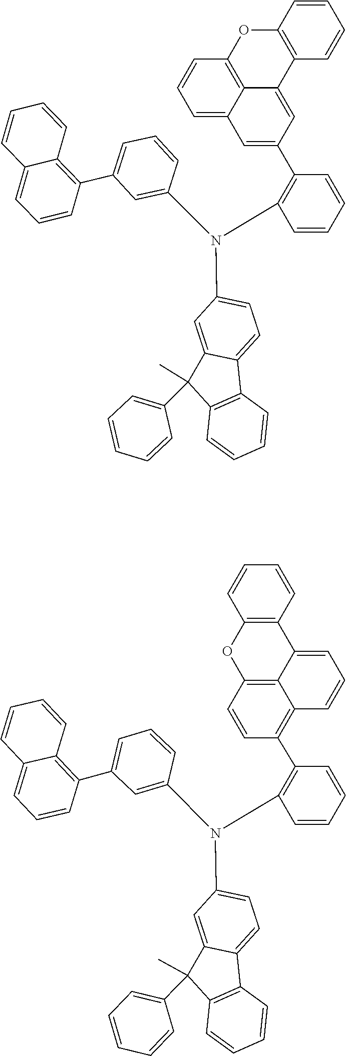 [SMILES: CC1(c2ccccc2)c2ccccc2-c2ccc(N(c3cccc(-c4cccc5ccccc45)c3)c3ccccc3-c3cc4c5c(cccc5c3)Oc3ccccc3-4)cc21 ; CC1(c2ccccc2)c2ccccc2-c2ccc(N(c3cccc(-c4cccc5ccccc45)c3)c3ccccc3-c3ccc4c5c(cccc35)-c3ccccc3O4)cc21]

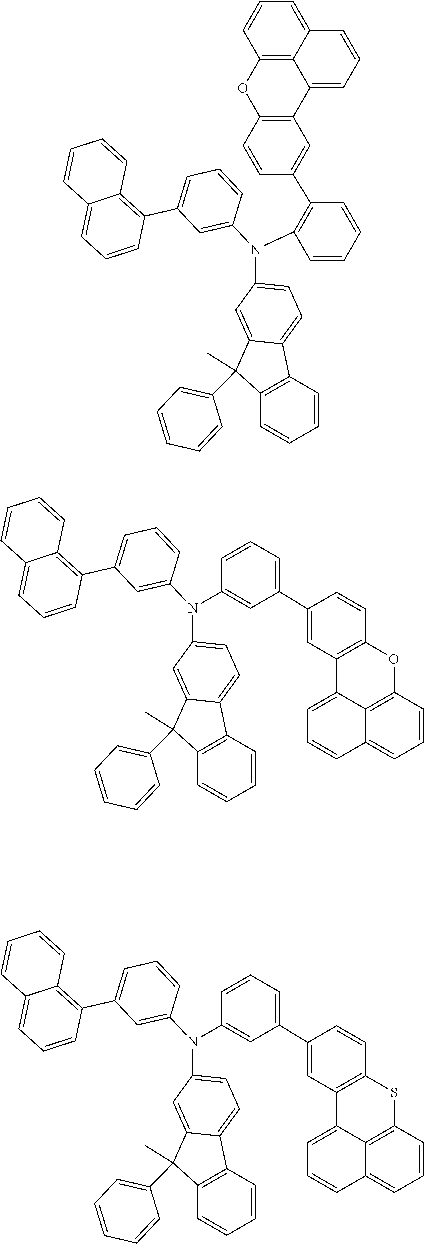 [SMILES: CC1(c2ccccc2)c2ccccc2-c2ccc(N(c3cccc(-c4cccc5ccccc45)c3)c3ccccc3-c3ccc4c(c3)-c3cccc5cccc(c35)O4)cc21 ; CC1(c2ccccc2)c2ccccc2-c2ccc(N(c3cccc(-c4ccc5c(c4)-c4cccc6cccc(c46)O5)c3)c3cccc(-c4cccc5ccccc45)c3)cc21 ; CC1(c2ccccc2)c2ccccc2-c2ccc(N(c3cccc(-c4ccc5c(c4)-c4cccc6cccc(c46)S5)c3)c3cccc(-c4cccc5ccccc45)c3)cc21]

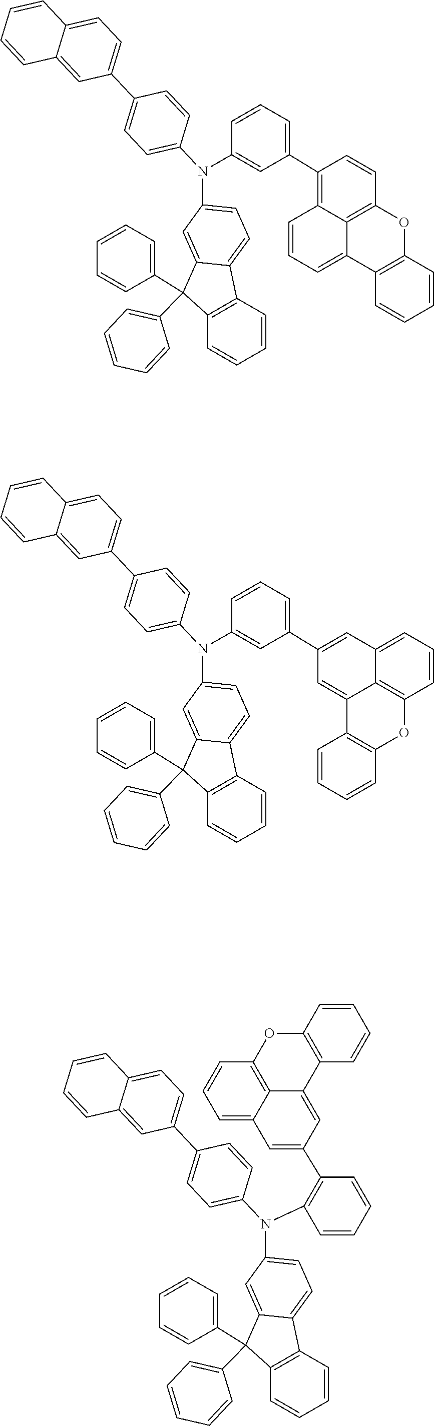 [SMILES: c1ccc(C2(c3ccccc3)c3ccccc3-c3ccc(N(c4ccc(-c5ccc6ccccc6c5)cc4)c4cccc(-c5ccc6c7c(cccc57)-c5ccccc5O6)c4)cc32)cc1 ; c1ccc(C2(c3ccccc3)c3ccccc3-c3ccc(N(c4ccc(-c5ccc6ccccc6c5)cc4)c4cccc(-c5cc6c7c(cccc7c5)Oc5ccccc5-6)c4)cc32)cc1 ; c1ccc(C2(c3ccccc3)c3ccccc3-c3ccc(N(c4ccc(-c5ccc6ccccc6c5)cc4)c4ccccc4-c4cc5c6c(cccc6c4)Oc4ccccc4-5)cc32)cc1]

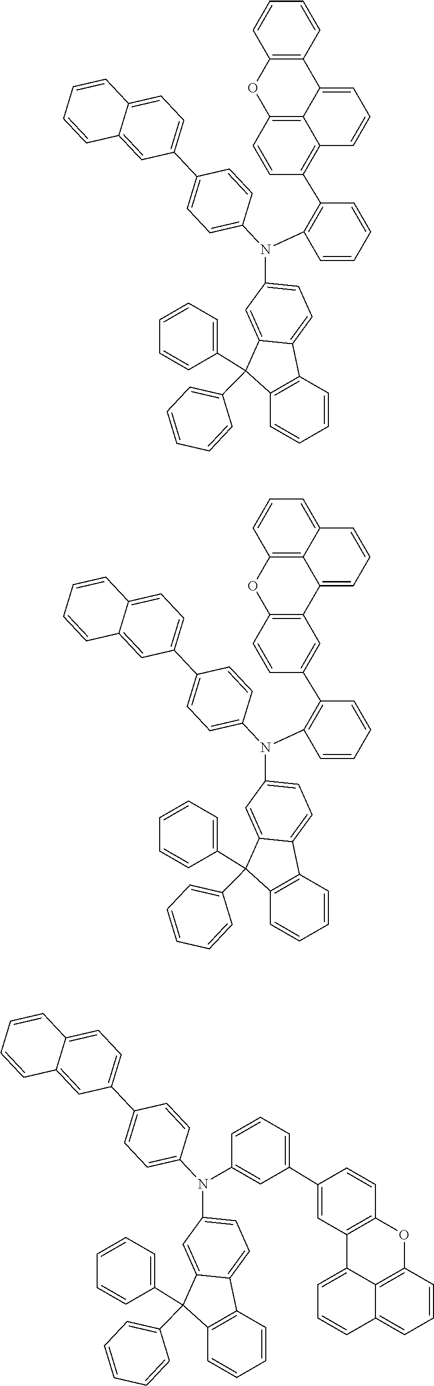 [SMILES: c1ccc(C2(c3ccccc3)c3ccccc3-c3ccc(N(c4ccc(-c5ccc6ccccc6c5)cc4)c4ccccc4-c4ccc5c6c(cccc46)-c4ccccc4O5)cc32)cc1 ; c1ccc(C2(c3ccccc3)c3ccccc3-c3ccc(N(c4ccc(-c5ccc6ccccc6c5)cc4)c4ccccc4-c4ccc5c(c4)-c4cccc6cccc(c46)O5)cc32)cc1 ; c1ccc(C2(c3ccccc3)c3ccccc3-c3ccc(N(c4ccc(-c5ccc6ccccc6c5)cc4)c4cccc(-c5ccc6c(c5)-c5cccc7cccc(c57)O6)c4)cc32)cc1]

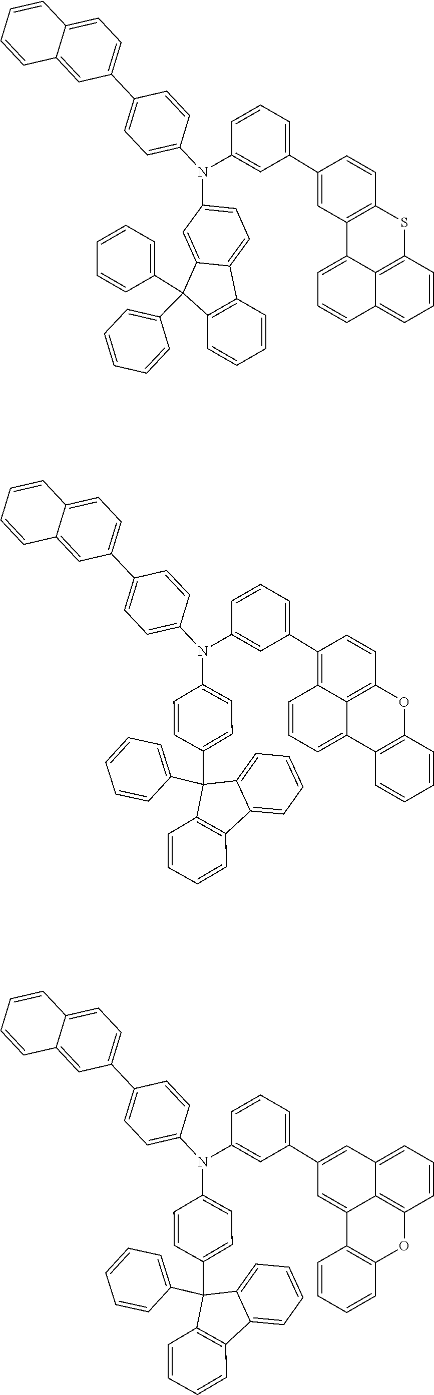 [SMILES: c1ccc(C2(c3ccccc3)c3ccccc3-c3ccc(N(c4ccc(-c5ccc6ccccc6c5)cc4)c4cccc(-c5ccc6c(c5)-c5cccc7cccc(c57)S6)c4)cc32)cc1 ; c1ccc(C2(c3ccc(N(c4ccc(-c5ccc6ccccc6c5)cc4)c4cccc(-c5ccc6c7c(cccc57)-c5ccccc5O6)c4)cc3)c3ccccc3-c3ccccc32)cc1 ; c1ccc(C2(c3ccc(N(c4ccc(-c5ccc6ccccc6c5)cc4)c4cccc(-c5cc6c7c(cccc7c5)Oc5ccccc5-6)c4)cc3)c3ccccc3-c3ccccc32)cc1]

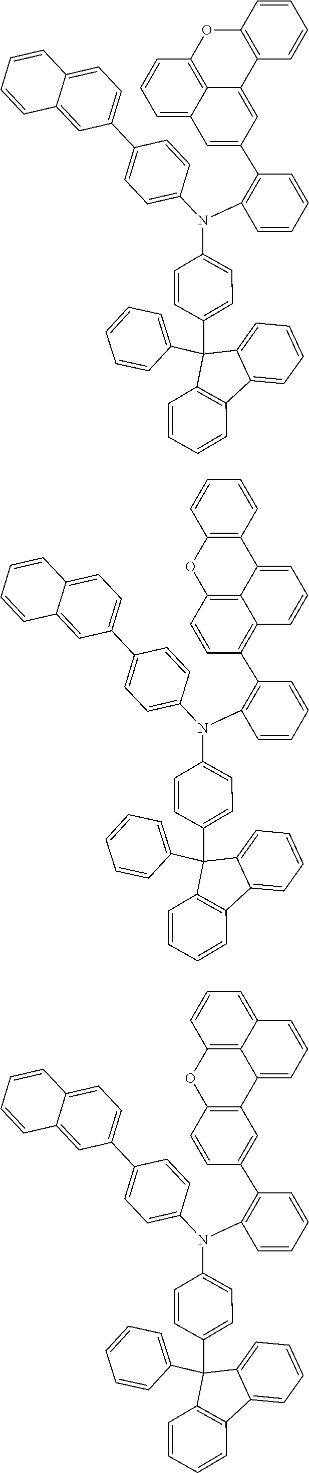

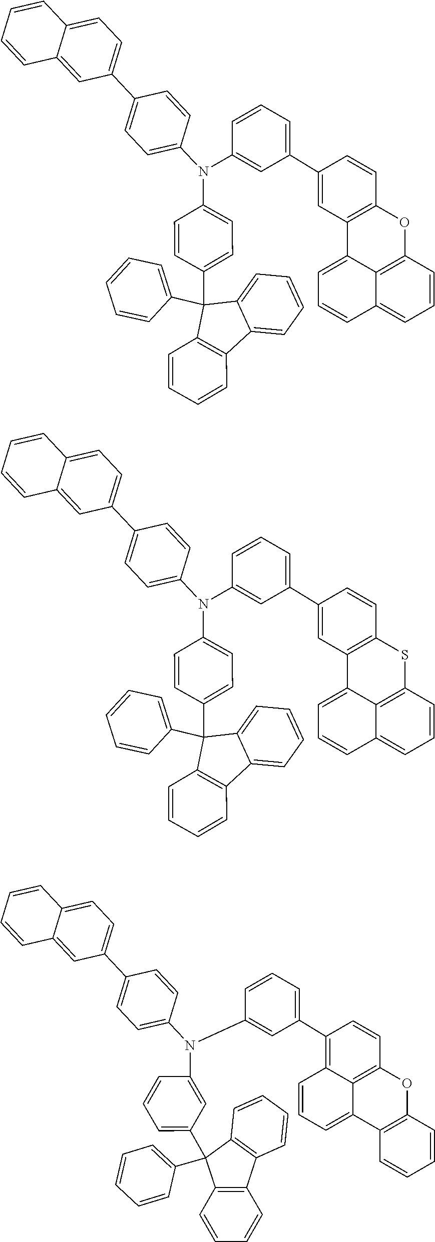 [SMILES: c1ccc(C2(c3ccc(N(c4ccc(-c5ccc6ccccc6c5)cc4)c4cccc(-c5ccc6c(c5)-c5cccc7cccc(c57)O6)c4)cc3)c3ccccc3-c3ccccc32)cc1 ; c1ccc(C2(c3ccc(N(c4ccc(-c5ccc6ccccc6c5)cc4)c4cccc(-c5ccc6c(c5)-c5cccc7cccc(c57)S6)c4)cc3)c3ccccc3-c3ccccc32)cc1 ; c1ccc(C2(c3cccc(N(c4ccc(-c5ccc6ccccc6c5)cc4)c4cccc(-c5ccc6c7c(cccc57)-c5ccccc5O6)c4)c3)c3ccccc3-c3ccccc32)cc1]

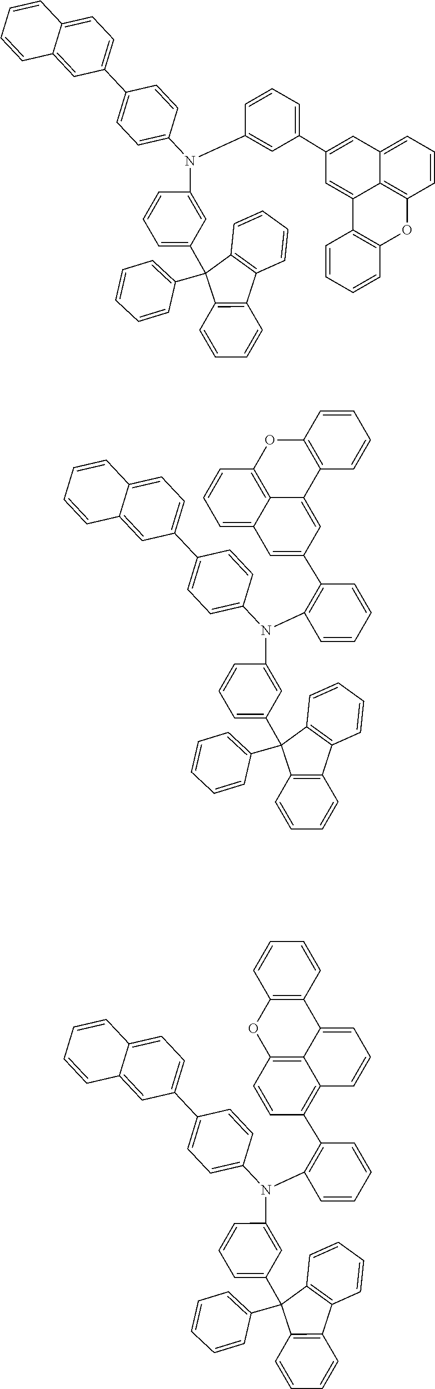 [SMILES: c1ccc(C2(c3cccc(N(c4ccc(-c5ccc6ccccc6c5)cc4)c4cccc(-c5cc6c7c(cccc7c5)Oc5ccccc5-6)c4)c3)c3ccccc3-c3ccccc32)cc1 ; c1ccc(C2(c3cccc(N(c4ccc(-c5ccc6ccccc6c5)cc4)c4ccccc4-c4cc5c6c(cccc6c4)Oc4ccccc4-5)c3)c3ccccc3-c3ccccc32)cc1 ; c1ccc(C2(c3cccc(N(c4ccc(-c5ccc6ccccc6c5)cc4)c4ccccc4-c4ccc5c6c(cccc46)-c4ccccc4O5)c3)c3ccccc3-c3ccccc32)cc1]

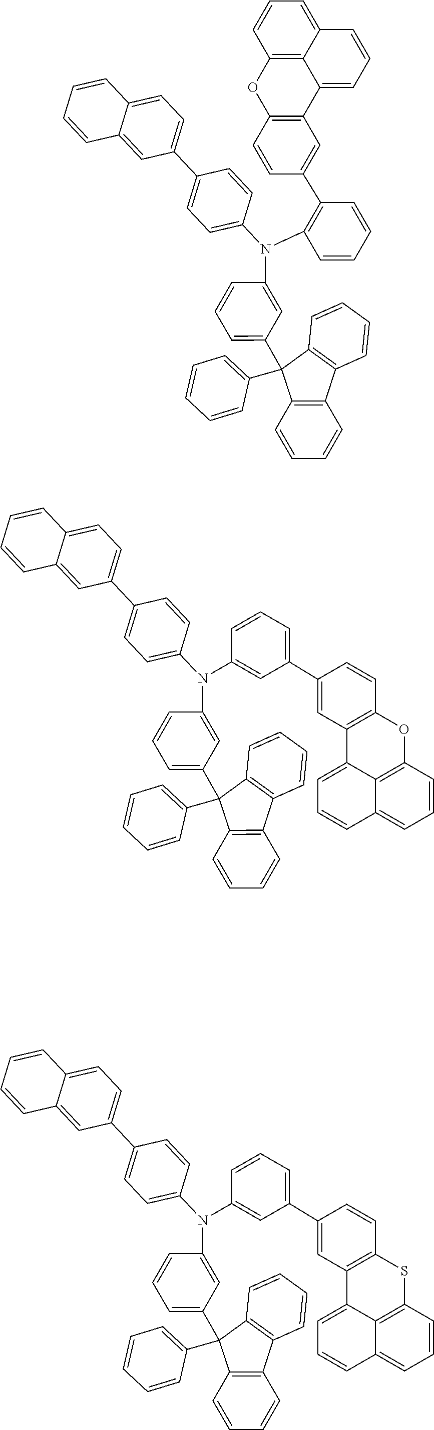 [SMILES: c1ccc(C2(c3cccc(N(c4ccc(-c5ccc6ccccc6c5)cc4)c4ccccc4-c4ccc5c(c4)-c4cccc6cccc(c46)O5)c3)c3ccccc3-c3ccccc32)cc1 ; c1ccc(C2(c3cccc(N(c4ccc(-c5ccc6ccccc6c5)cc4)c4cccc(-c5ccc6c(c5)-c5cccc7cccc(c57)O6)c4)c3)c3ccccc3-c3ccccc32)cc1 ; c1ccc(C2(c3cccc(N(c4ccc(-c5ccc6ccccc6c5)cc4)c4cccc(-c5ccc6c(c5)-c5cccc7cccc(c57)S6)c4)c3)c3ccccc3-c3ccccc32)cc1]

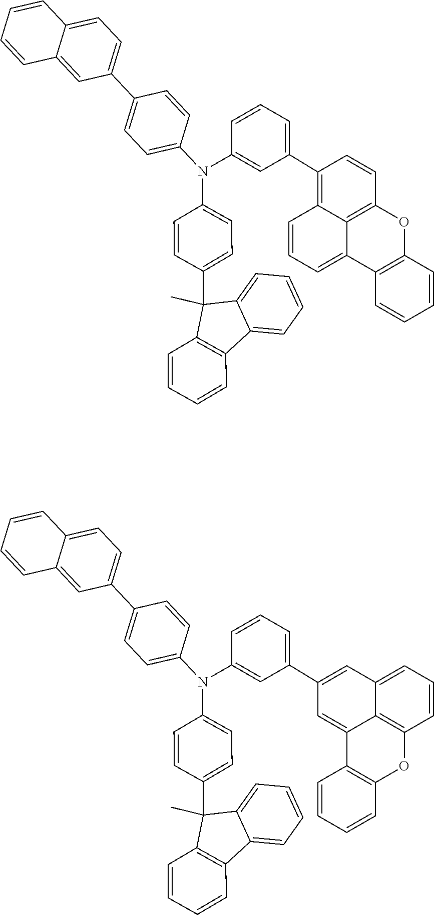 [SMILES: CC1(c2ccc(N(c3ccc(-c4ccc5ccccc5c4)cc3)c3cccc(-c4ccc5c6c(cccc46)-c4ccccc4O5)c3)cc2)c2ccccc2-c2ccccc21 ; CC1(c2ccc(N(c3ccc(-c4ccc5ccccc5c4)cc3)c3cccc(-c4cc5c6c(cccc6c4)Oc4ccccc4-5)c3)cc2)c2ccccc2-c2ccccc21]

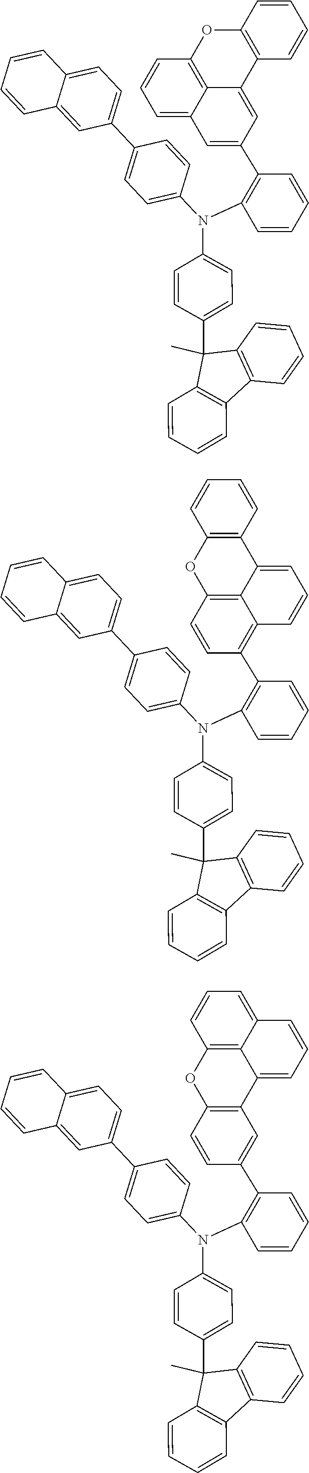

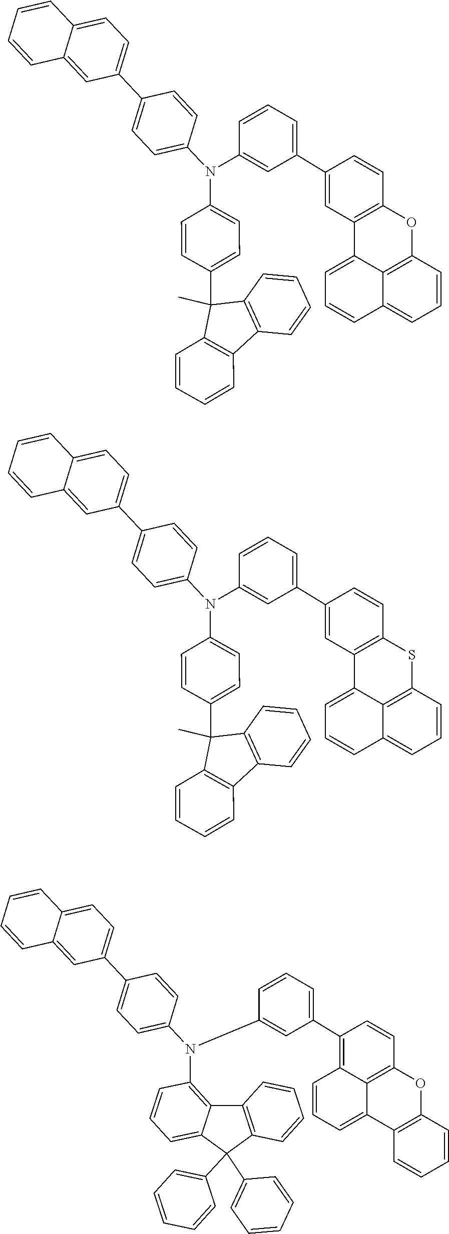 [SMILES: CC1(c2ccc(N(c3ccc(-c4ccc5ccccc5c4)cc3)c3cccc(-c4ccc5c(c4)-c4cccc6cccc(c46)O5)c3)cc2)c2ccccc2-c2ccccc21 ; CC1(c2ccc(N(c3ccc(-c4ccc5ccccc5c4)cc3)c3cccc(-c4ccc5c(c4)-c4cccc6cccc(c46)S5)c3)cc2)c2ccccc2-c2ccccc21 ; c1ccc(C2(c3ccccc3)c3ccccc3-c3c(N(c4ccc(-c5ccc6ccccc6c5)cc4)c4cccc(-c5ccc6c7c(cccc57)-c5ccccc5O6)c4)cccc32)cc1]

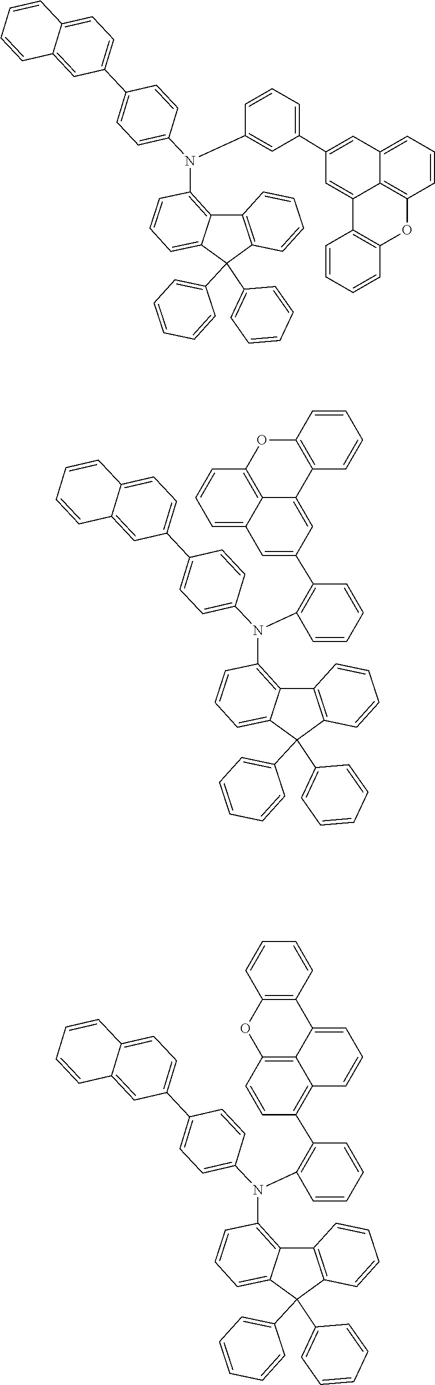 [SMILES: c1ccc(C2(c3ccccc3)c3ccccc3-c3c(N(c4ccc(-c5ccc6ccccc6c5)cc4)c4cccc(-c5cc6c7c(cccc7c5)Oc5ccccc5-6)c4)cccc32)cc1 ; c1ccc(C2(c3ccccc3)c3ccccc3-c3c(N(c4ccc(-c5ccc6ccccc6c5)cc4)c4ccccc4-c4cc5c6c(cccc6c4)Oc4ccccc4-5)cccc32)cc1 ; c1ccc(C2(c3ccccc3)c3ccccc3-c3c(N(c4ccc(-c5ccc6ccccc6c5)cc4)c4ccccc4-c4ccc5c6c(cccc46)-c4ccccc4O5)cccc32)cc1]

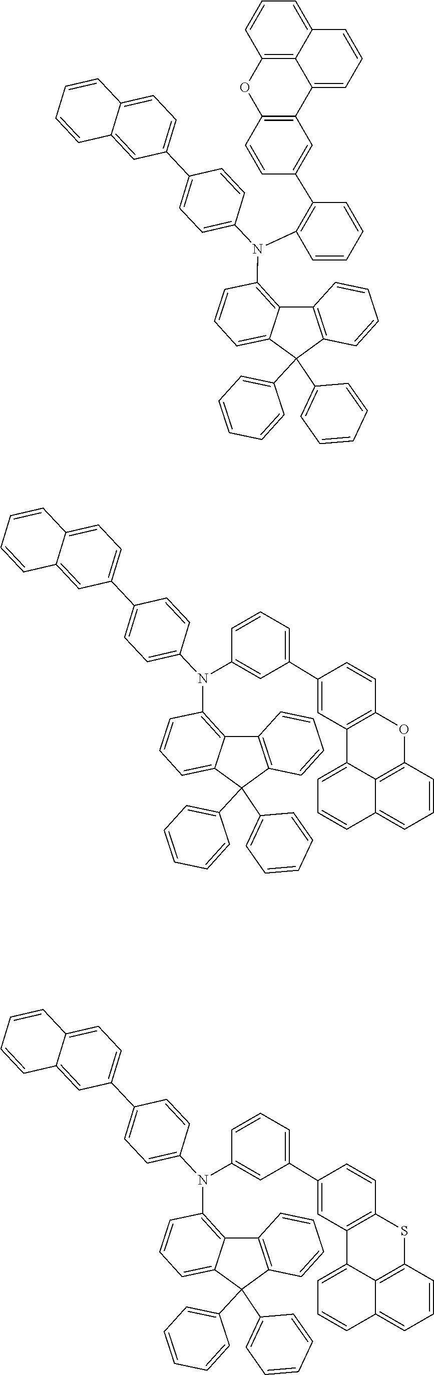 [SMILES: c1ccc(C2(c3ccccc3)c3ccccc3-c3c(N(c4ccc(-c5ccc6ccccc6c5)cc4)c4ccccc4-c4ccc5c(c4)-c4cccc6cccc(c46)O5)cccc32)cc1 ; c1ccc(C2(c3ccccc3)c3ccccc3-c3c(N(c4ccc(-c5ccc6ccccc6c5)cc4)c4cccc(-c5ccc6c(c5)-c5cccc7cccc(c57)O6)c4)cccc32)cc1 ; c1ccc(C2(c3ccccc3)c3ccccc3-c3c(N(c4ccc(-c5ccc6ccccc6c5)cc4)c4cccc(-c5ccc6c(c5)-c5cccc7cccc(c57)S6)c4)cccc32)cc1]

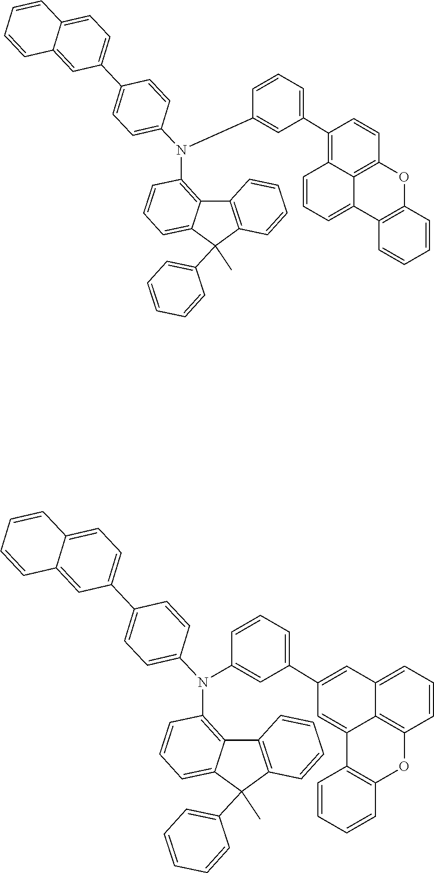 [SMILES: CC1(c2ccccc2)c2ccccc2-c2c(N(c3ccc(-c4ccc5ccccc5c4)cc3)c3cccc(-c4ccc5c6c(cccc46)-c4ccccc4O5)c3)cccc21 ; CC1(c2ccccc2)c2ccccc2-c2c(N(c3ccc(-c4ccc5ccccc5c4)cc3)c3cccc(-c4cc5c6c(cccc6c4)Oc4ccccc4-5)c3)cccc21]

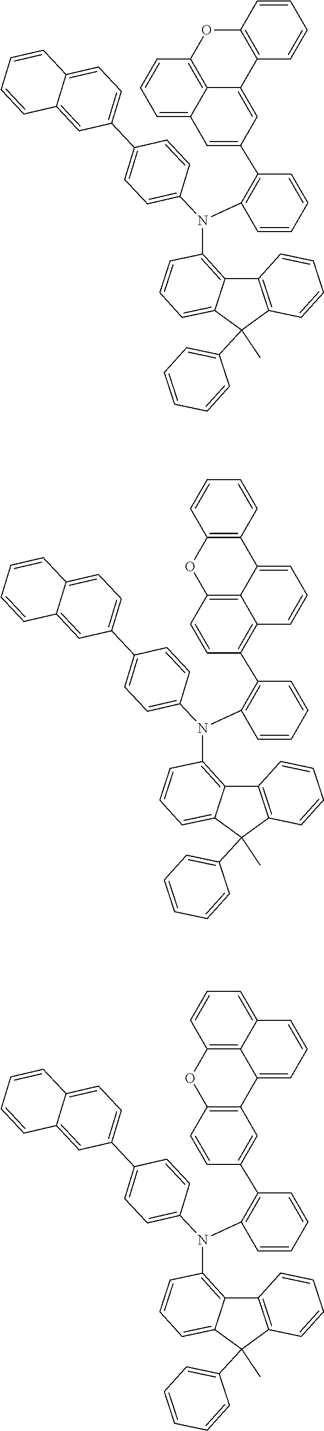 [SMILES: CC1(c2ccccc2)c2ccccc2-c2c(N(c3ccc(-c4ccc5ccccc5c4)cc3)c3ccccc3-c3cc4c5c(cccc5c3)Oc3ccccc3-4)cccc21 ; CC1(c2ccccc2)c2ccccc2-c2c(N(c3ccc(-c4ccc5ccccc5c4)cc3)c3ccccc3-c3ccc4c5c(cccc35)-c3ccccc3O4)cccc21 ; CC1(c2ccccc2)c2ccccc2-c2c(N(c3ccc(-c4ccc5ccccc5c4)cc3)c3ccccc3-c3ccc4c(c3)-c3cccc5cccc(c35)O4)cccc21]

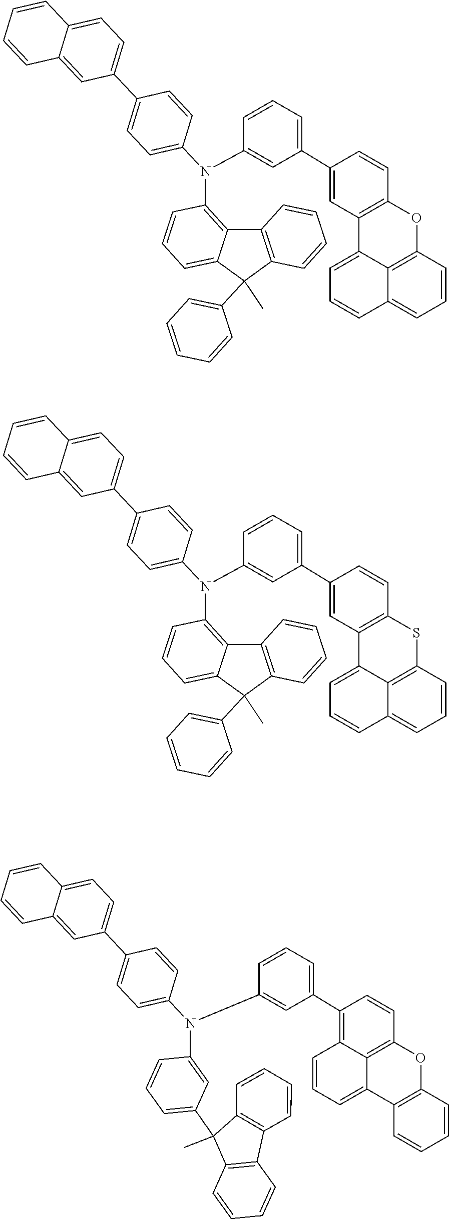 [SMILES: CC1(c2ccccc2)c2ccccc2-c2c(N(c3ccc(-c4ccc5ccccc5c4)cc3)c3cccc(-c4ccc5c(c4)-c4cccc6cccc(c46)O5)c3)cccc21 ; CC1(c2ccccc2)c2ccccc2-c2c(N(c3ccc(-c4ccc5ccccc5c4)cc3)c3cccc(-c4ccc5c(c4)-c4cccc6cccc(c46)S5)c3)cccc21 ; CC1(c2cccc(N(c3ccc(-c4ccc5ccccc5c4)cc3)c3cccc(-c4ccc5c6c(cccc46)-c4ccccc4O5)c3)c2)c2ccccc2-c2ccccc21]

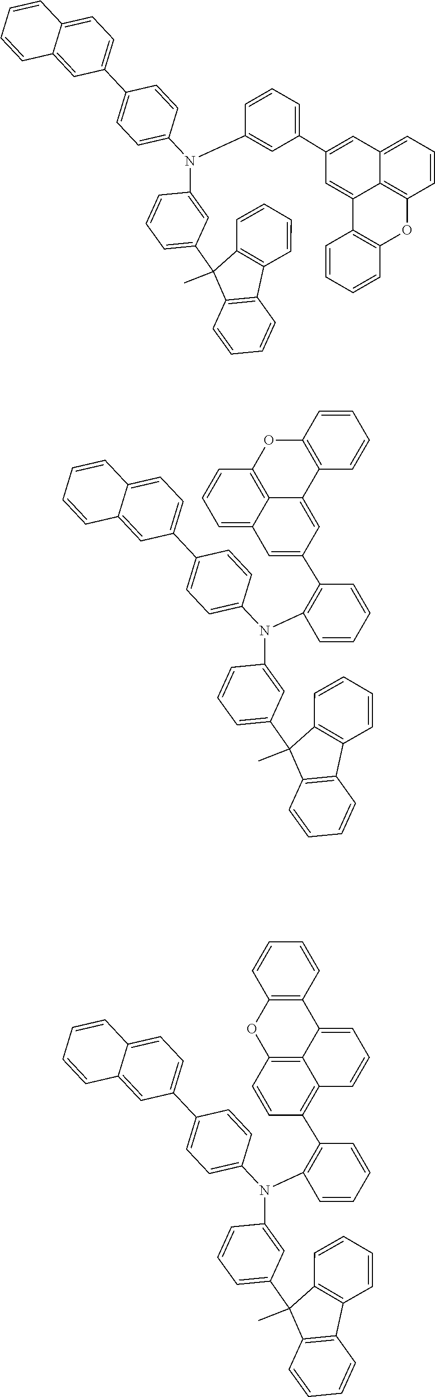 [SMILES: CC1(c2cccc(N(c3ccc(-c4ccc5ccccc5c4)cc3)c3cccc(-c4cc5c6c(cccc6c4)Oc4ccccc4-5)c3)c2)c2ccccc2-c2ccccc21 ; CC1(c2cccc(N(c3ccc(-c4ccc5ccccc5c4)cc3)c3ccccc3-c3cc4c5c(cccc5c3)Oc3ccccc3-4)c2)c2ccccc2-c2ccccc21 ; CC1(c2cccc(N(c3ccc(-c4ccc5ccccc5c4)cc3)c3ccccc3-c3ccc4c5c(cccc35)-c3ccccc3O4)c2)c2ccccc2-c2ccccc21]

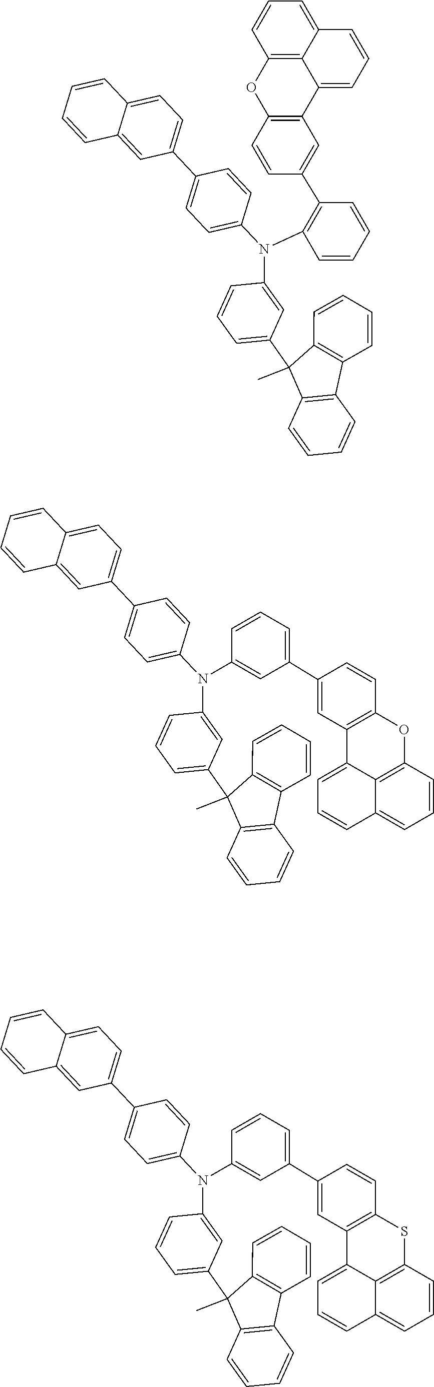 [SMILES: CC1(c2cccc(N(c3ccc(-c4ccc5ccccc5c4)cc3)c3ccccc3-c3ccc4c(c3)-c3cccc5cccc(c35)O4)c2)c2ccccc2-c2ccccc21 ; CC1(c2cccc(N(c3ccc(-c4ccc5ccccc5c4)cc3)c3cccc(-c4ccc5c(c4)-c4cccc6cccc(c46)O5)c3)c2)c2ccccc2-c2ccccc21 ; CC1(c2cccc(N(c3ccc(-c4ccc5ccccc5c4)cc3)c3cccc(-c4ccc5c(c4)-c4cccc6cccc(c46)S5)c3)c2)c2ccccc2-c2ccccc21]

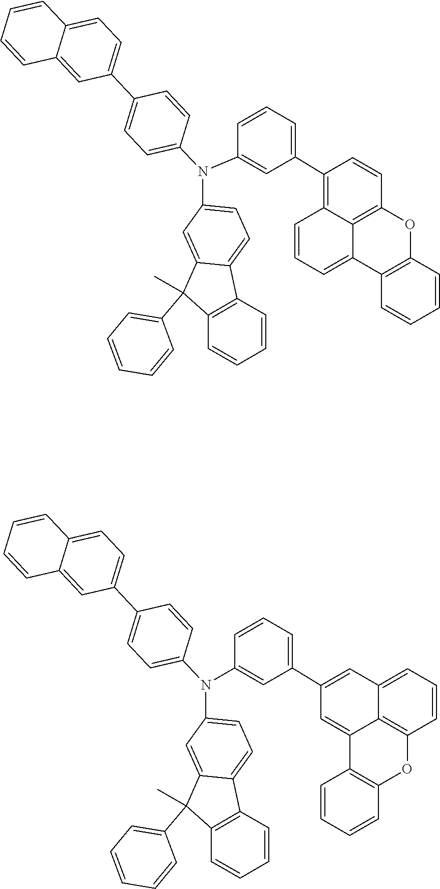 [SMILES: CC1(c2ccccc2)c2ccccc2-c2ccc(N(c3ccc(-c4ccc5ccccc5c4)cc3)c3cccc(-c4ccc5c6c(cccc46)-c4ccccc4O5)c3)cc21 ; CC1(c2ccccc2)c2ccccc2-c2ccc(N(c3ccc(-c4ccc5ccccc5c4)cc3)c3cccc(-c4cc5c6c(cccc6c4)Oc4ccccc4-5)c3)cc21]

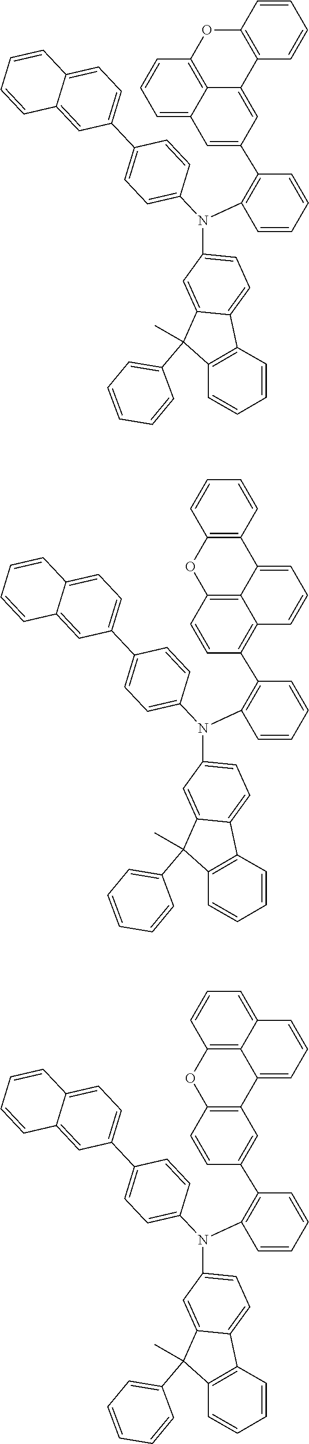 [SMILES: CC1(c2ccccc2)c2ccccc2-c2ccc(N(c3ccc(-c4ccc5ccccc5c4)cc3)c3ccccc3-c3cc4c5c(cccc5c3)Oc3ccccc3-4)cc21 ; CC1(c2ccccc2)c2ccccc2-c2ccc(N(c3ccc(-c4ccc5ccccc5c4)cc3)c3ccccc3-c3ccc4c5c(cccc35)-c3ccccc3O4)cc21 ; CC1(c2ccccc2)c2ccccc2-c2ccc(N(c3ccc(-c4ccc5ccccc5c4)cc3)c3ccccc3-c3ccc4c(c3)-c3cccc5cccc(c35)O4)cc21]

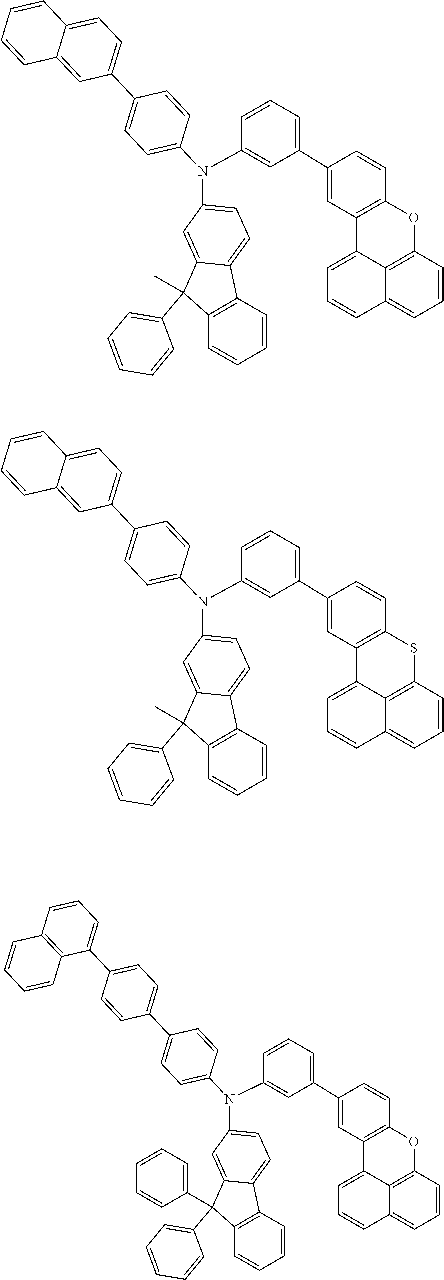 [SMILES: CC1(c2ccccc2)c2ccccc2-c2ccc(N(c3ccc(-c4ccc5ccccc5c4)cc3)c3cccc(-c4ccc5c(c4)-c4cccc6cccc(c46)O5)c3)cc21 ; CC1(c2ccccc2)c2ccccc2-c2ccc(N(c3ccc(-c4ccc5ccccc5c4)cc3)c3cccc(-c4ccc5c(c4)-c4cccc6cccc(c46)S5)c3)cc21 ; c1ccc(C2(c3ccccc3)c3ccccc3-c3ccc(N(c4ccc(-c5ccc(-c6cccc7ccccc67)cc5)cc4)c4cccc(-c5ccc6c(c5)-c5cccc7cccc(c57)O6)c4)cc32)cc1]

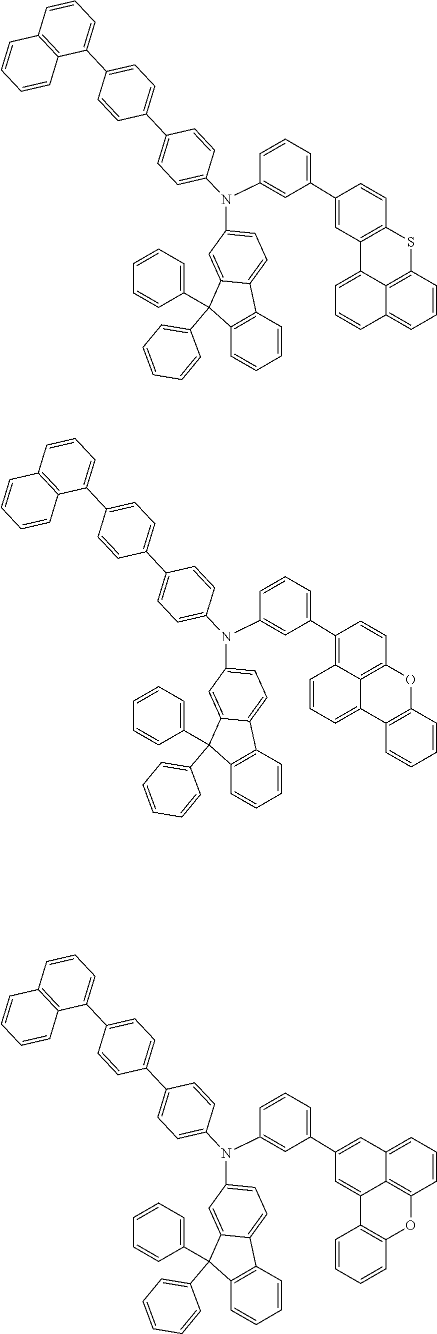 [SMILES: c1ccc(C2(c3ccccc3)c3ccccc3-c3ccc(N(c4ccc(-c5ccc(-c6cccc7ccccc67)cc5)cc4)c4cccc(-c5ccc6c(c5)-c5cccc7cccc(c57)S6)c4)cc32)cc1 ; c1ccc(C2(c3ccccc3)c3ccccc3-c3ccc(N(c4ccc(-c5ccc(-c6cccc7ccccc67)cc5)cc4)c4cccc(-c5ccc6c7c(cccc57)-c5ccccc5O6)c4)cc32)cc1 ; c1ccc(C2(c3ccccc3)c3ccccc3-c3ccc(N(c4ccc(-c5ccc(-c6cccc7ccccc67)cc5)cc4)c4cccc(-c5cc6c7c(cccc7c5)Oc5ccccc5-6)c4)cc32)cc1]

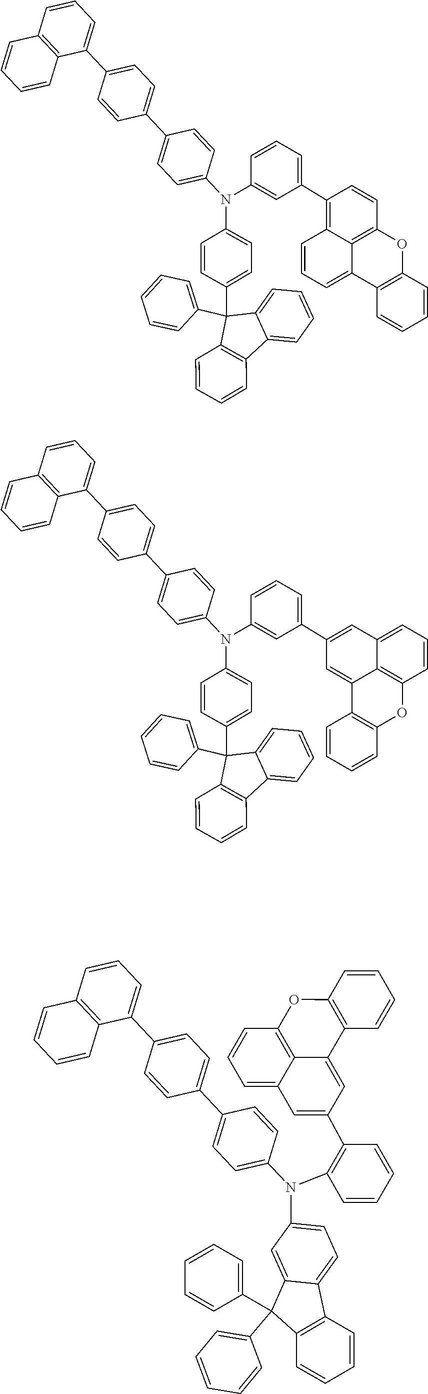 [SMILES: c1ccc(C2(c3ccc(N(c4ccc(-c5ccc(-c6cccc7ccccc67)cc5)cc4)c4cccc(-c5ccc6c7c(cccc57)-c5ccccc5O6)c4)cc3)c3ccccc3-c3ccccc32)cc1 ; c1ccc(C2(c3ccc(N(c4ccc(-c5ccc(-c6cccc7ccccc67)cc5)cc4)c4cccc(-c5cc6c7c(cccc7c5)Oc5ccccc5-6)c4)cc3)c3ccccc3-c3ccccc32)cc1 ; c1ccc(C2(c3ccccc3)c3ccccc3-c3ccc(N(c4ccc(-c5ccc(-c6cccc7ccccc67)cc5)cc4)c4ccccc4-c4cc5c6c(cccc6c4)Oc4ccccc4-5)cc32)cc1]

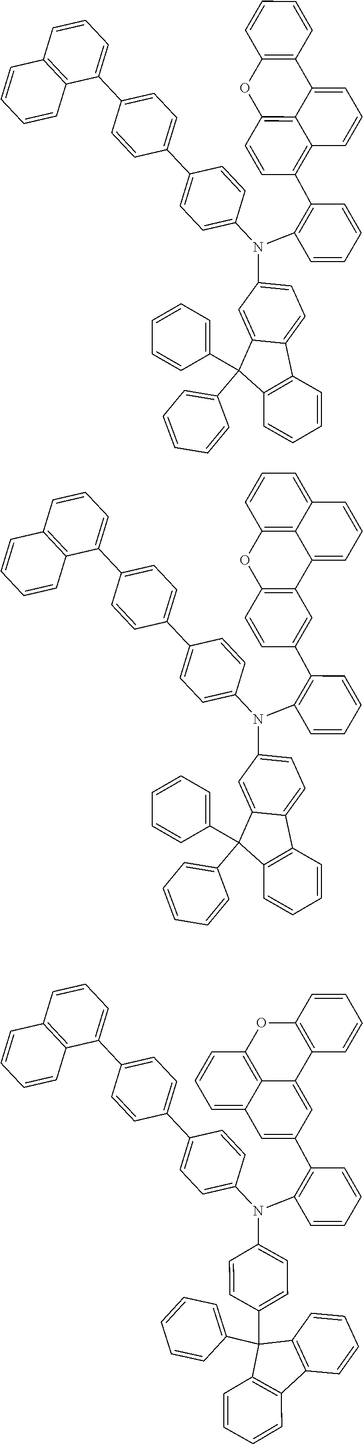

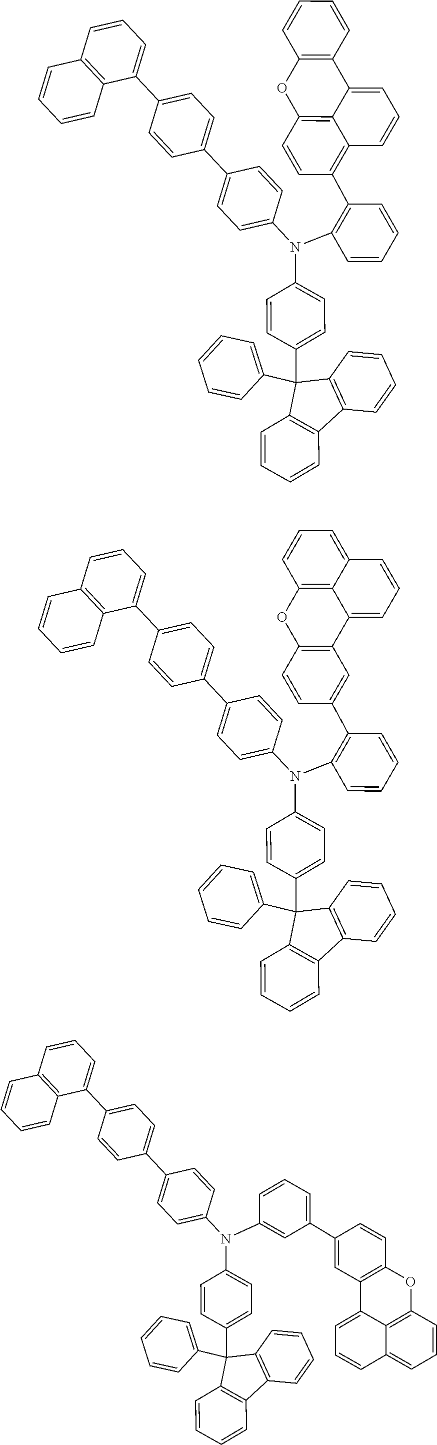 [SMILES: c1ccc(C2(c3ccc(N(c4ccc(-c5ccc(-c6cccc7ccccc67)cc5)cc4)c4ccccc4-c4ccc5c6c(cccc46)-c4ccccc4O5)cc3)c3ccccc3-c3ccccc32)cc1 ; c1ccc(C2(c3ccc(N(c4ccc(-c5ccc(-c6cccc7ccccc67)cc5)cc4)c4ccccc4-c4ccc5c(c4)-c4cccc6cccc(c46)O5)cc3)c3ccccc3-c3ccccc32)cc1 ; c1ccc(C2(c3ccc(N(c4ccc(-c5ccc(-c6cccc7ccccc67)cc5)cc4)c4cccc(-c5ccc6c(c5)-c5cccc7cccc(c57)O6)c4)cc3)c3ccccc3-c3ccccc32)cc1]

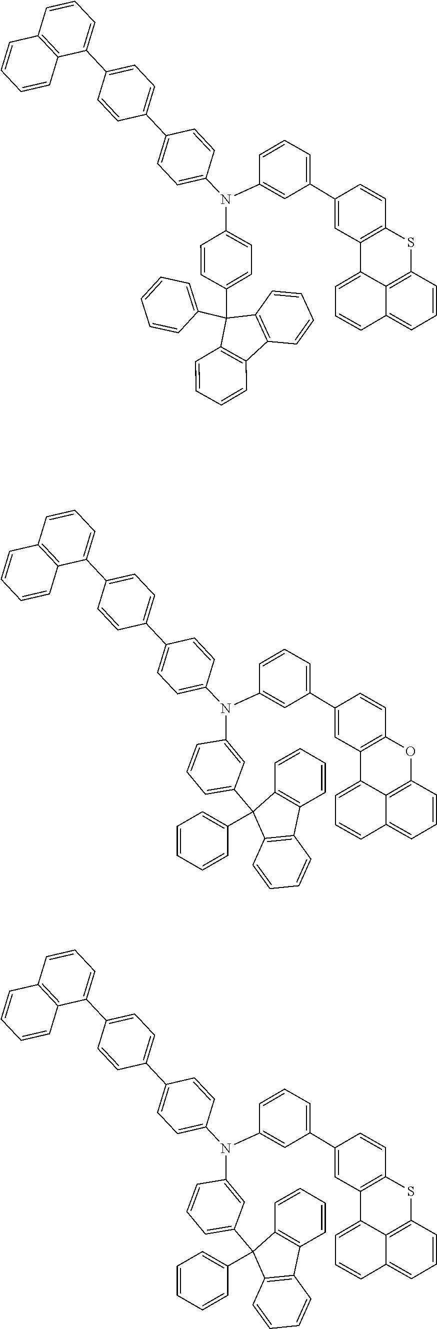 [SMILES: c1ccc(C2(c3ccc(N(c4ccc(-c5ccc(-c6cccc7ccccc67)cc5)cc4)c4cccc(-c5ccc6c(c5)-c5cccc7cccc(c57)S6)c4)cc3)c3ccccc3-c3ccccc32)cc1 ; c1ccc(C2(c3cccc(N(c4ccc(-c5ccc(-c6cccc7ccccc67)cc5)cc4)c4cccc(-c5ccc6c(c5)-c5cccc7cccc(c57)O6)c4)c3)c3ccccc3-c3ccccc32)cc1 ; c1ccc(C2(c3cccc(N(c4ccc(-c5ccc(-c6cccc7ccccc67)cc5)cc4)c4cccc(-c5ccc6c(c5)-c5cccc7cccc(c57)S6)c4)c3)c3ccccc3-c3ccccc32)cc1]

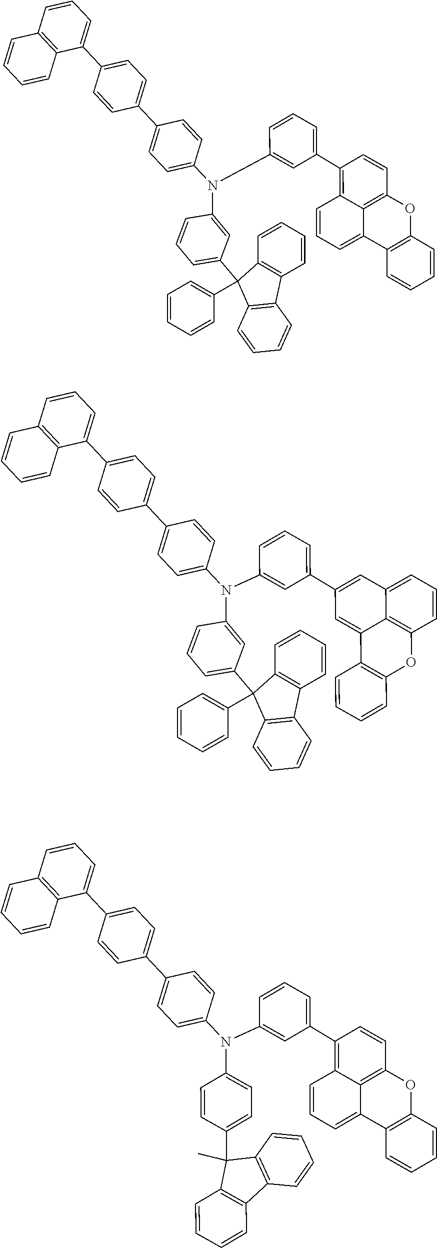 [SMILES: c1ccc(C2(c3cccc(N(c4ccc(-c5ccc(-c6cccc7ccccc67)cc5)cc4)c4cccc(-c5ccc6c7c(cccc57)-c5ccccc5O6)c4)c3)c3ccccc3-c3ccccc32)cc1 ; c1ccc(C2(c3cccc(N(c4ccc(-c5ccc(-c6cccc7ccccc67)cc5)cc4)c4cccc(-c5cc6c7c(cccc7c5)Oc5ccccc5-6)c4)c3)c3ccccc3-c3ccccc32)cc1 ; CC1(c2ccc(N(c3ccc(-c4ccc(-c5cccc6ccccc56)cc4)cc3)c3cccc(-c4ccc5c6c(cccc46)-c4ccccc4O5)c3)cc2)c2ccccc2-c2ccccc21]

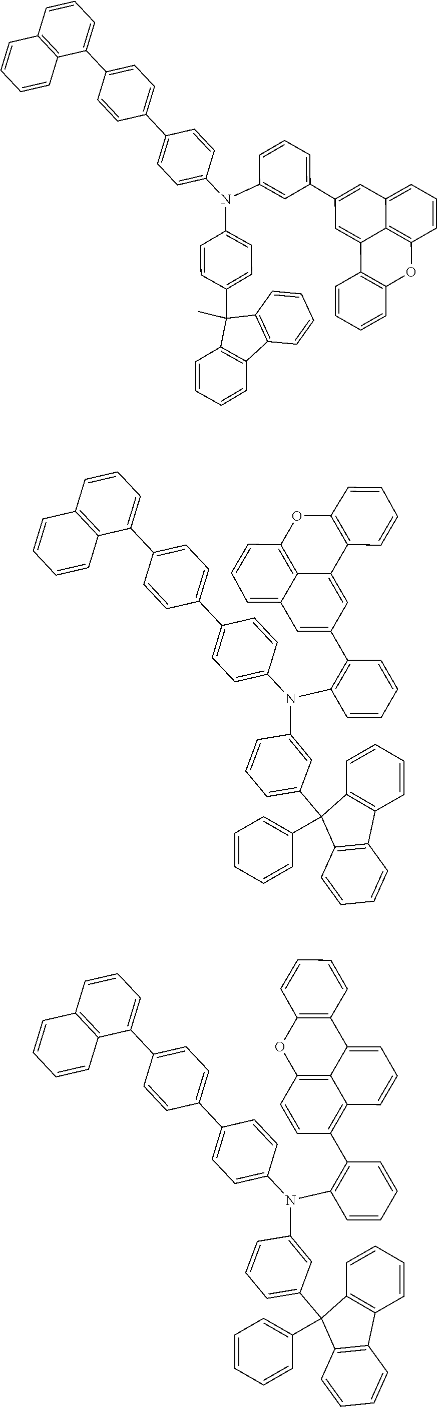 [SMILES: CC1(c2ccc(N(c3ccc(-c4ccc(-c5cccc6ccccc56)cc4)cc3)c3cccc(-c4cc5c6c(cccc6c4)Oc4ccccc4-5)c3)cc2)c2ccccc2-c2ccccc21 ; c1ccc(C2(c3cccc(N(c4ccc(-c5ccc(-c6cccc7ccccc67)cc5)cc4)c4ccccc4-c4cc5c6c(cccc6c4)Oc4ccccc4-5)c3)c3ccccc3-c3ccccc32)cc1 ; c1ccc(C2(c3cccc(N(c4ccc(-c5ccc(-c6cccc7ccccc67)cc5)cc4)c4ccccc4-c4ccc5c6c(cccc46)-c4ccccc4O5)c3)c3ccccc3-c3ccccc32)cc1]

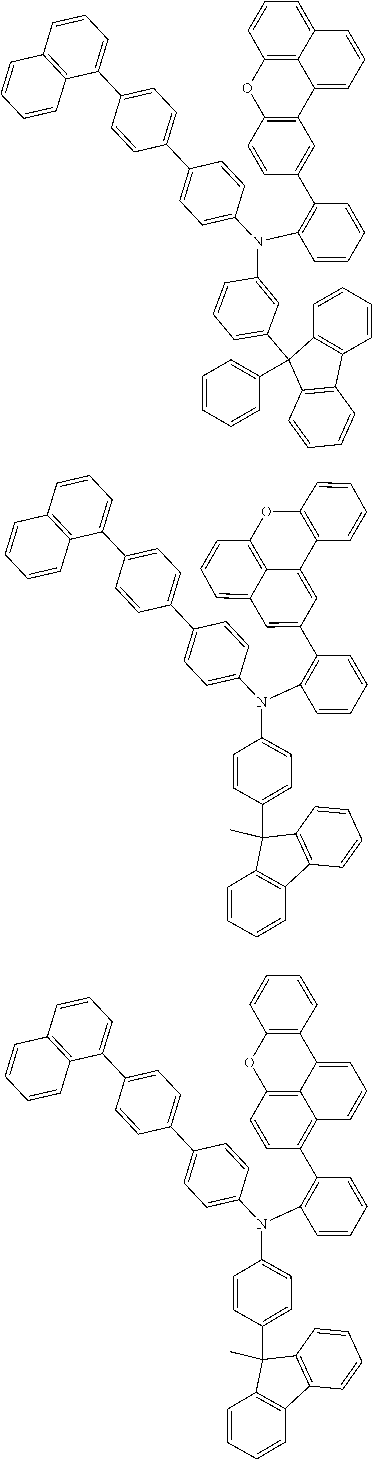

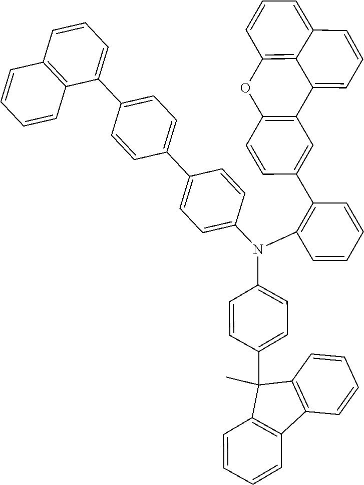 [SMILES: CC1(c2ccc(N(c3ccc(-c4ccc(-c5cccc6ccccc56)cc4)cc3)c3ccccc3-c3ccc4c(c3)-c3cccc5cccc(c35)O4)cc2)c2ccccc2-c2ccccc21]

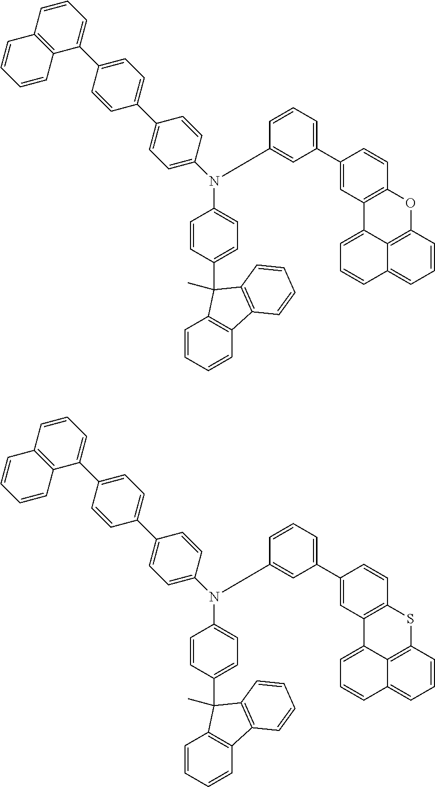 [SMILES: CC1(c2ccc(N(c3ccc(-c4ccc(-c5cccc6ccccc56)cc4)cc3)c3cccc(-c4ccc5c(c4)-c4cccc6cccc(c46)O5)c3)cc2)c2ccccc2-c2ccccc21 ; CC1(c2ccc(N(c3ccc(-c4ccc(-c5cccc6ccccc56)cc4)cc3)c3cccc(-c4ccc5c(c4)-c4cccc6cccc(c46)S5)c3)cc2)c2ccccc2-c2ccccc21]

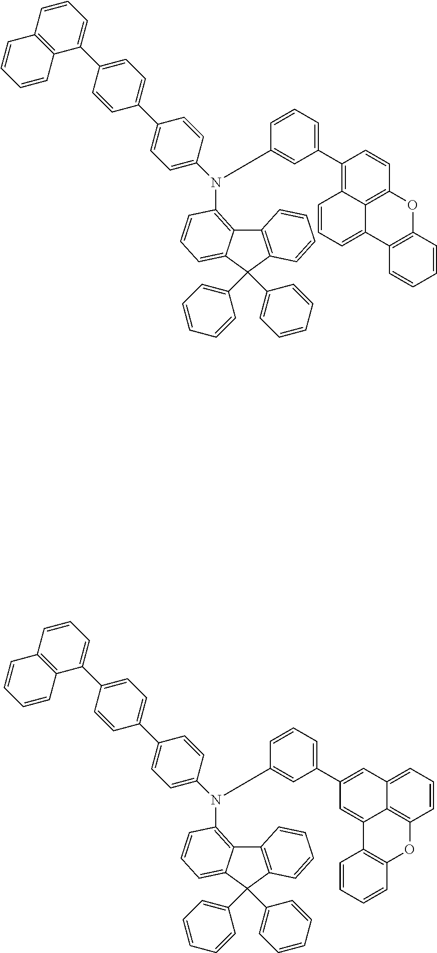 [SMILES: c1ccc(C2(c3ccccc3)c3ccccc3-c3c(N(c4ccc(-c5ccc(-c6cccc7ccccc67)cc5)cc4)c4cccc(-c5ccc6c7c(cccc57)-c5ccccc5O6)c4)cccc32)cc1 ; c1ccc(C2(c3ccccc3)c3ccccc3-c3c(N(c4ccc(-c5ccc(-c6cccc7ccccc67)cc5)cc4)c4cccc(-c5cc6c7c(cccc7c5)Oc5ccccc5-6)c4)cccc32)cc1]

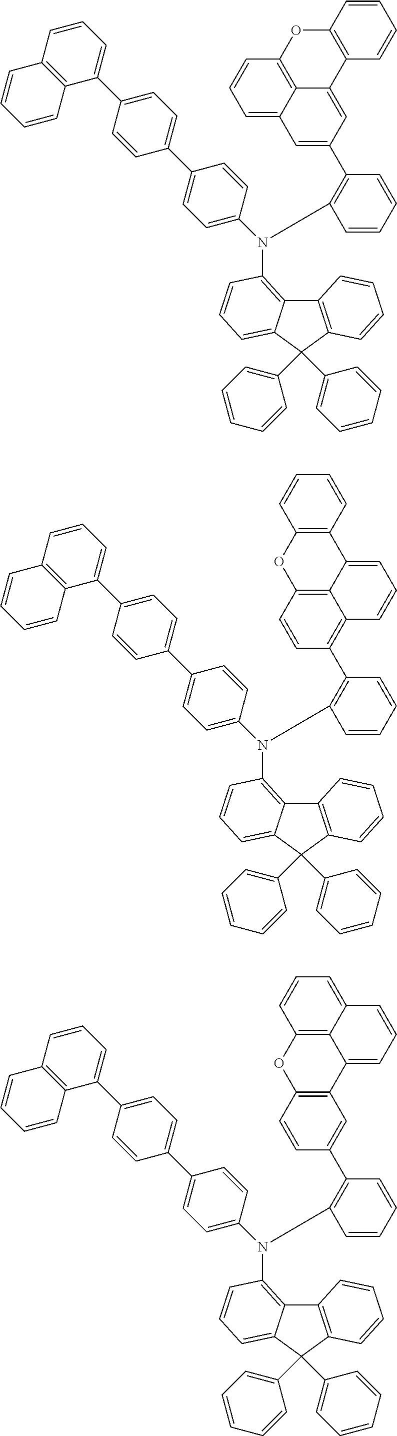

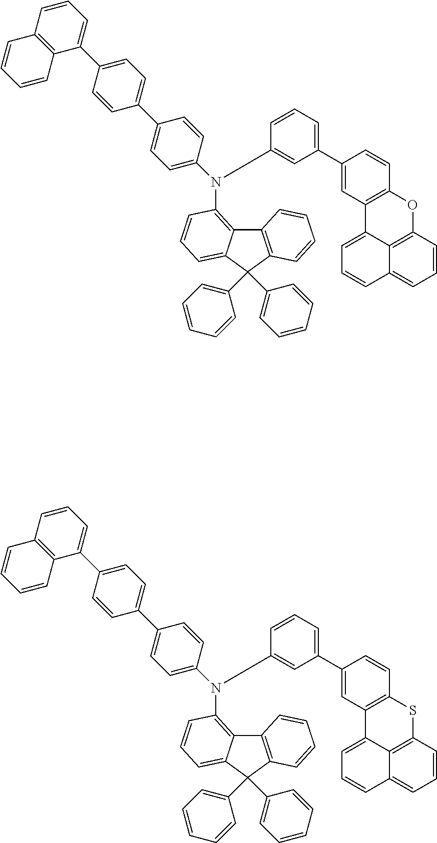 [SMILES: c1ccc(C2(c3ccccc3)c3ccccc3-c3c(N(c4ccc(-c5ccc(-c6cccc7ccccc67)cc5)cc4)c4cccc(-c5ccc6c(c5)-c5cccc7cccc(c57)O6)c4)cccc32)cc1 ; c1ccc(C2(c3ccccc3)c3ccccc3-c3c(N(c4ccc(-c5ccc(-c6cccc7ccccc67)cc5)cc4)c4cccc(-c5ccc6c(c5)-c5cccc7cccc(c57)S6)c4)cccc32)cc1]

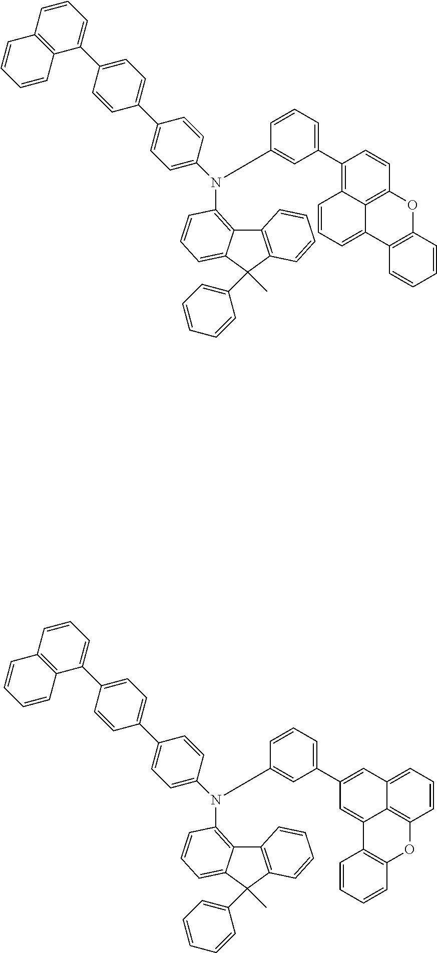 [SMILES: CC1(c2ccccc2)c2ccccc2-c2c(N(c3ccc(-c4ccc(-c5cccc6ccccc56)cc4)cc3)c3cccc(-c4ccc5c6c(cccc46)-c4ccccc4O5)c3)cccc21 ; CC1(c2ccccc2)c2ccccc2-c2c(N(c3ccc(-c4ccc(-c5cccc6ccccc56)cc4)cc3)c3cccc(-c4cc5c6c(cccc6c4)Oc4ccccc4-5)c3)cccc21]

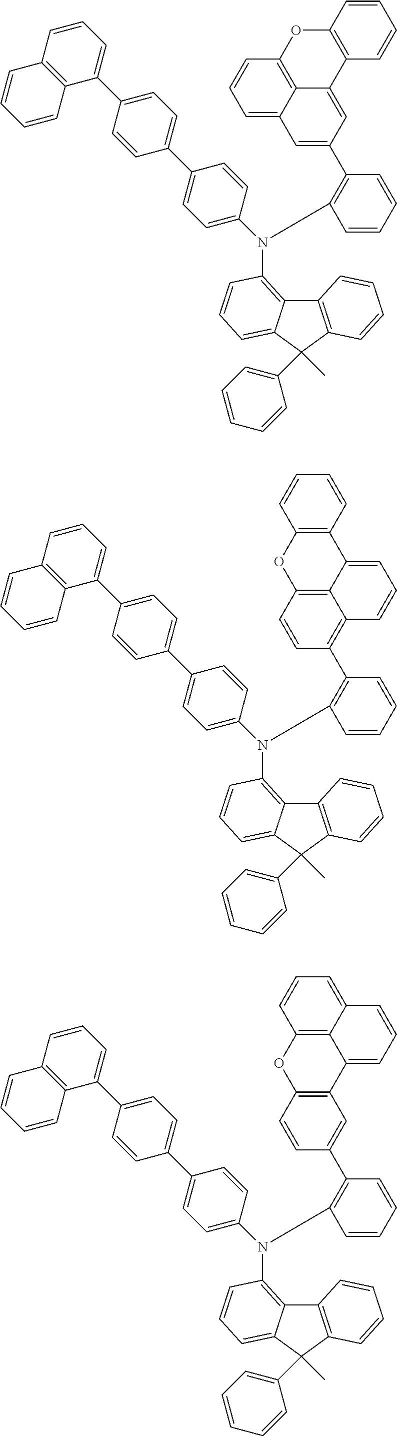 [SMILES: CC1(c2ccccc2)c2ccccc2-c2c(N(c3ccc(-c4ccc(-c5cccc6ccccc56)cc4)cc3)c3ccccc3-c3cc4c5c(cccc5c3)Oc3ccccc3-4)cccc21 ; CC1(c2ccccc2)c2ccccc2-c2c(N(c3ccc(-c4ccc(-c5cccc6ccccc56)cc4)cc3)c3ccccc3-c3ccc4c5c(cccc35)-c3ccccc3O4)cccc21 ; CC1(c2ccccc2)c2ccccc2-c2c(N(c3ccc(-c4ccc(-c5cccc6ccccc56)cc4)cc3)c3ccccc3-c3ccc4c(c3)-c3cccc5cccc(c35)O4)cccc21]

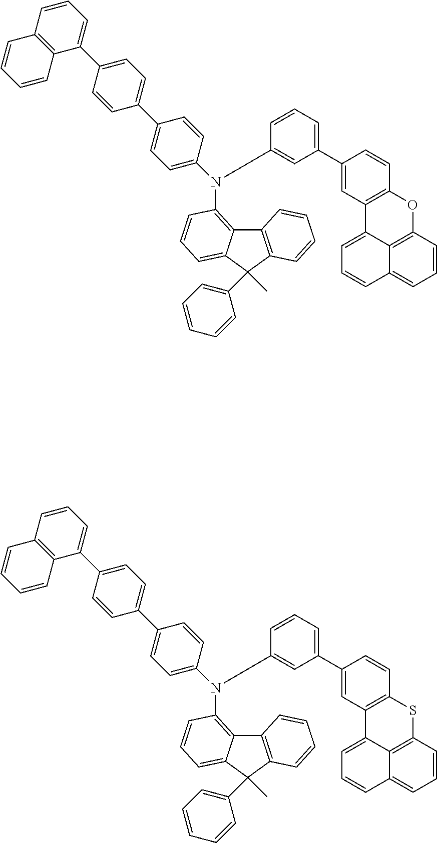 [SMILES: CC1(c2ccccc2)c2ccccc2-c2c(N(c3ccc(-c4ccc(-c5cccc6ccccc56)cc4)cc3)c3cccc(-c4ccc5c(c4)-c4cccc6cccc(c46)O5)c3)cccc21 ; CC1(c2ccccc2)c2ccccc2-c2c(N(c3ccc(-c4ccc(-c5cccc6ccccc56)cc4)cc3)c3cccc(-c4ccc5c(c4)-c4cccc6cccc(c46)S5)c3)cccc21]

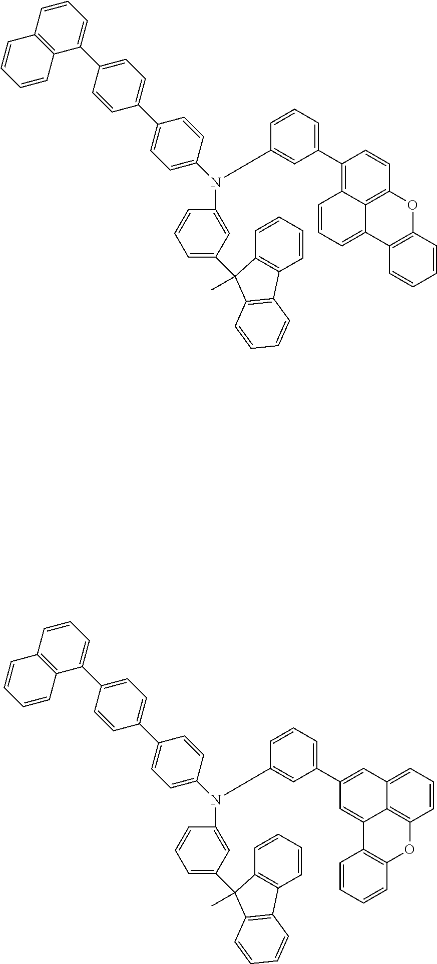 [SMILES: CC1(c2cccc(N(c3ccc(-c4ccc(-c5cccc6ccccc56)cc4)cc3)c3cccc(-c4ccc5c6c(cccc46)-c4ccccc4O5)c3)c2)c2ccccc2-c2ccccc21 ; CC1(c2cccc(N(c3ccc(-c4ccc(-c5cccc6ccccc56)cc4)cc3)c3cccc(-c4cc5c6c(cccc6c4)Oc4ccccc4-5)c3)c2)c2ccccc2-c2ccccc21]

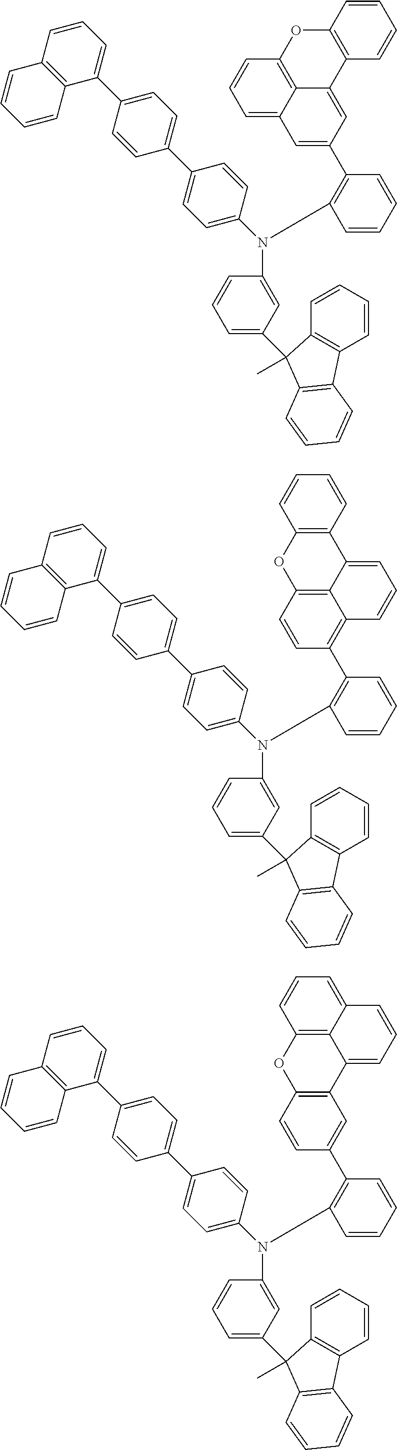

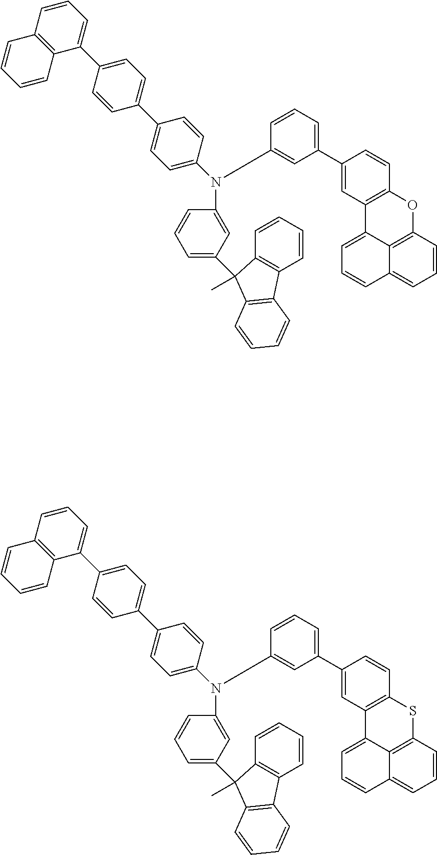 [SMILES: CC1(c2cccc(N(c3ccc(-c4ccc(-c5cccc6ccccc56)cc4)cc3)c3cccc(-c4ccc5c(c4)-c4cccc6cccc(c46)O5)c3)c2)c2ccccc2-c2ccccc21 ; CC1(c2cccc(N(c3ccc(-c4ccc(-c5cccc6ccccc56)cc4)cc3)c3cccc(-c4ccc5c(c4)-c4cccc6cccc(c46)S5)c3)c2)c2ccccc2-c2ccccc21]

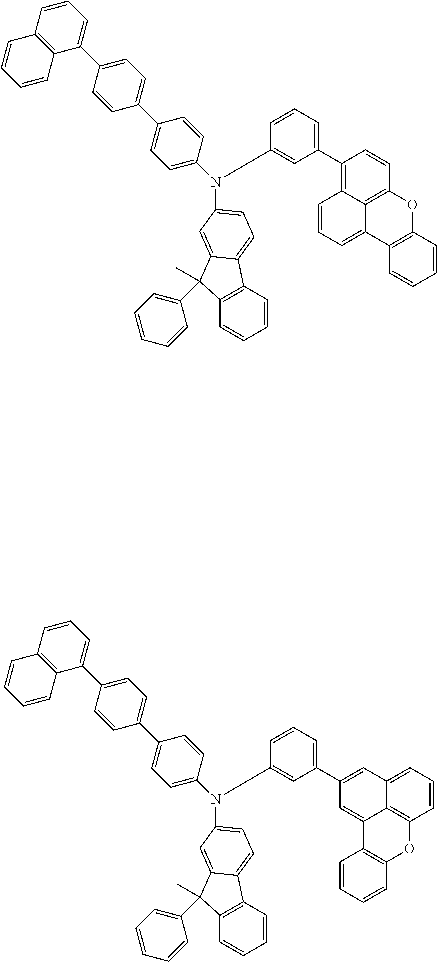 [SMILES: CC1(c2ccccc2)c2ccccc2-c2ccc(N(c3ccc(-c4ccc(-c5cccc6ccccc56)cc4)cc3)c3cccc(-c4ccc5c6c(cccc46)-c4ccccc4O5)c3)cc21 ; CC1(c2ccccc2)c2ccccc2-c2ccc(N(c3ccc(-c4ccc(-c5cccc6ccccc56)cc4)cc3)c3cccc(-c4cc5c6c(cccc6c4)Oc4ccccc4-5)c3)cc21]

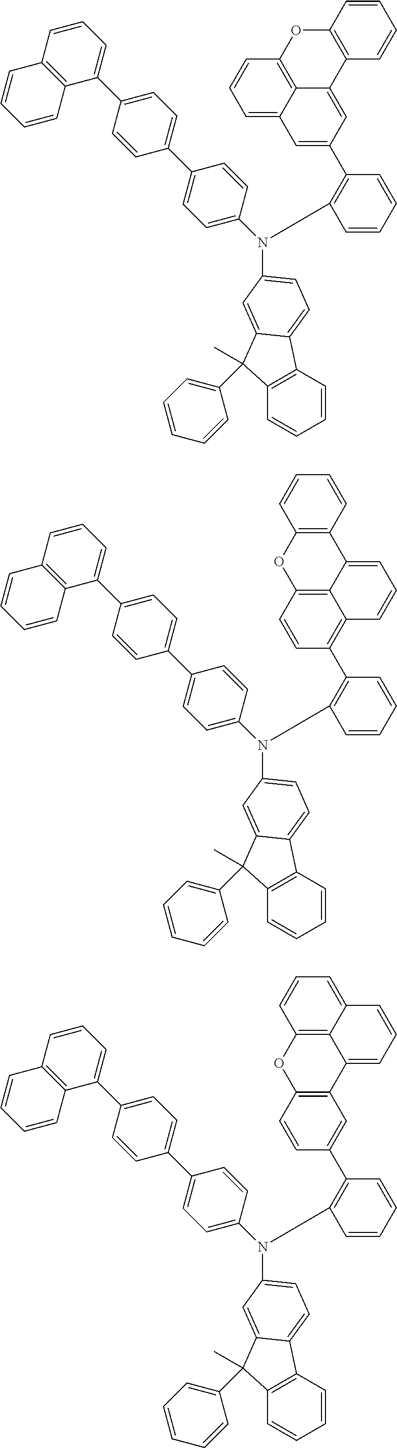

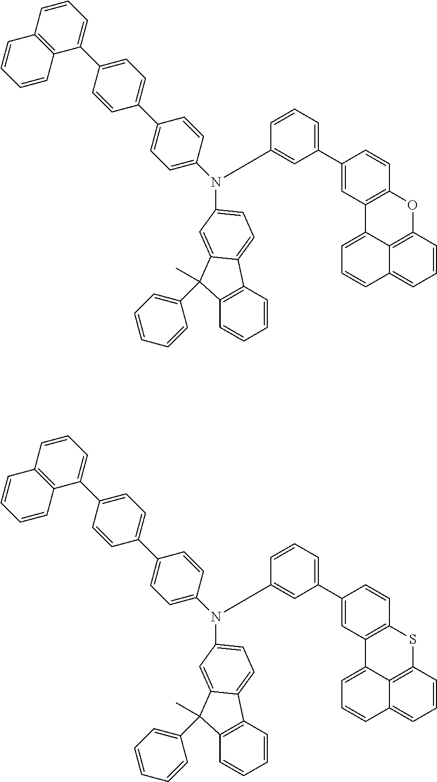 [SMILES: CC1(c2ccccc2)c2ccccc2-c2ccc(N(c3ccc(-c4ccc(-c5cccc6ccccc56)cc4)cc3)c3cccc(-c4ccc5c(c4)-c4cccc6cccc(c46)O5)c3)cc21 ; CC1(c2ccccc2)c2ccccc2-c2ccc(N(c3ccc(-c4ccc(-c5cccc6ccccc56)cc4)cc3)c3cccc(-c4ccc5c(c4)-c4cccc6cccc(c46)S5)c3)cc21]

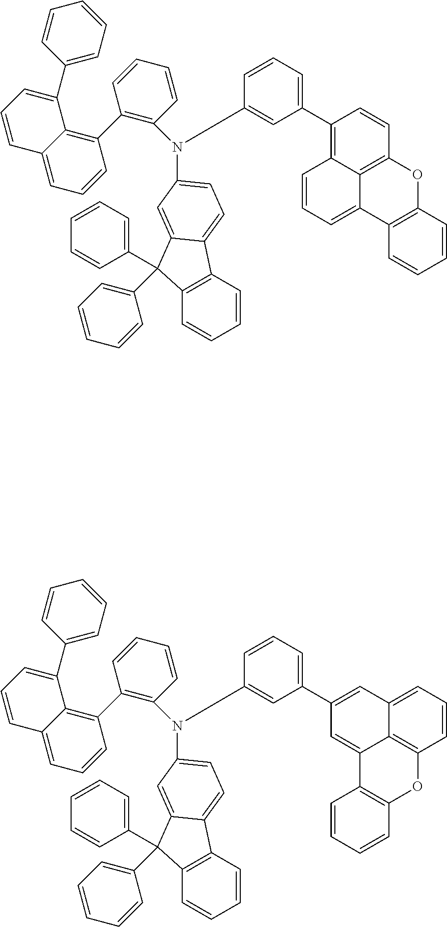 [SMILES: c1ccc(-c2cccc3cccc(-c4ccccc4N(c4cccc(-c5ccc6c7c(cccc57)-c5ccccc5O6)c4)c4ccc5c(c4)C(c4ccccc4)(c4ccccc4)c4ccccc4-5)c23)cc1 ; c1ccc(-c2cccc3cccc(-c4ccccc4N(c4cccc(-c5cc6c7c(cccc7c5)Oc5ccccc5-6)c4)c4ccc5c(c4)C(c4ccccc4)(c4ccccc4)c4ccccc4-5)c23)cc1]

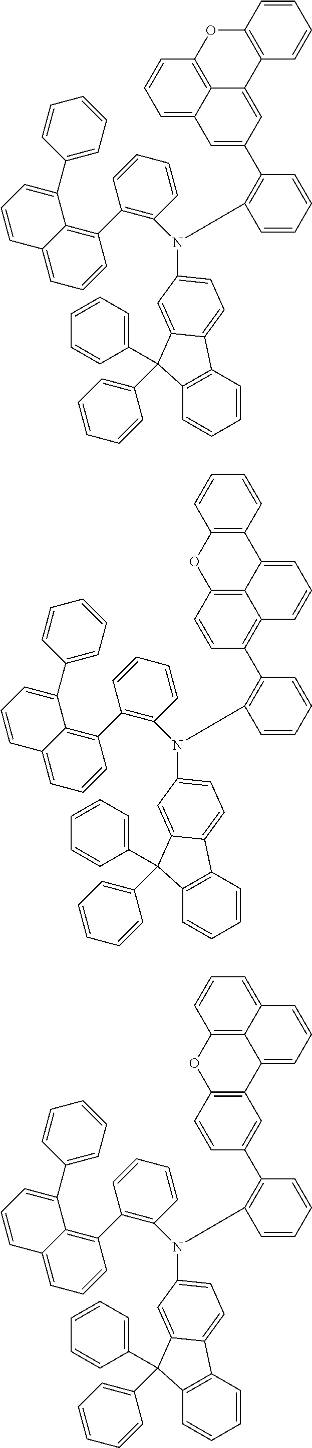

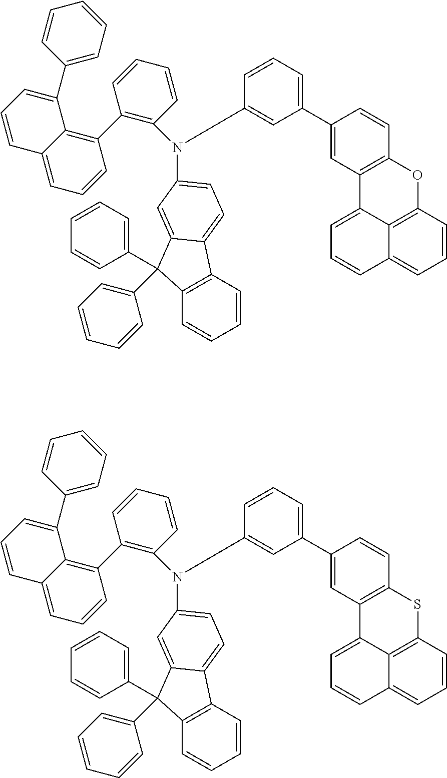 [SMILES: c1ccc(-c2cccc3cccc(-c4ccccc4N(c4cccc(-c5ccc6c(c5)-c5cccc7cccc(c57)O6)c4)c4ccc5c(c4)C(c4ccccc4)(c4ccccc4)c4ccccc4-5)c23)cc1 ; c1ccc(-c2cccc3cccc(-c4ccccc4N(c4cccc(-c5ccc6c(c5)-c5cccc7cccc(c57)S6)c4)c4ccc5c(c4)C(c4ccccc4)(c4ccccc4)c4ccccc4-5)c23)cc1]

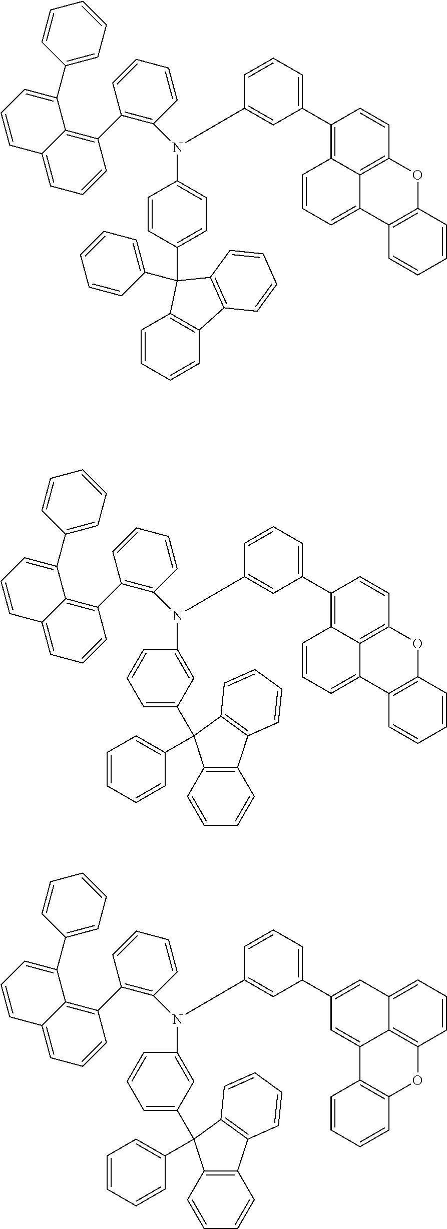 [SMILES: c1ccc(-c2cccc3cccc(-c4ccccc4N(c4ccc(C5(c6ccccc6)c6ccccc6-c6ccccc65)cc4)c4cccc(-c5ccc6c7c(cccc57)-c5ccccc5O6)c4)c23)cc1 ; c1ccc(-c2cccc3cccc(-c4ccccc4N(c4cccc(-c5ccc6c7c(cccc57)-c5ccccc5O6)c4)c4cccc(C5(c6ccccc6)c6ccccc6-c6ccccc65)c4)c23)cc1 ; c1ccc(-c2cccc3cccc(-c4ccccc4N(c4cccc(-c5cc6c7c(cccc7c5)Oc5ccccc5-6)c4)c4cccc(C5(c6ccccc6)c6ccccc6-c6ccccc65)c4)c23)cc1]

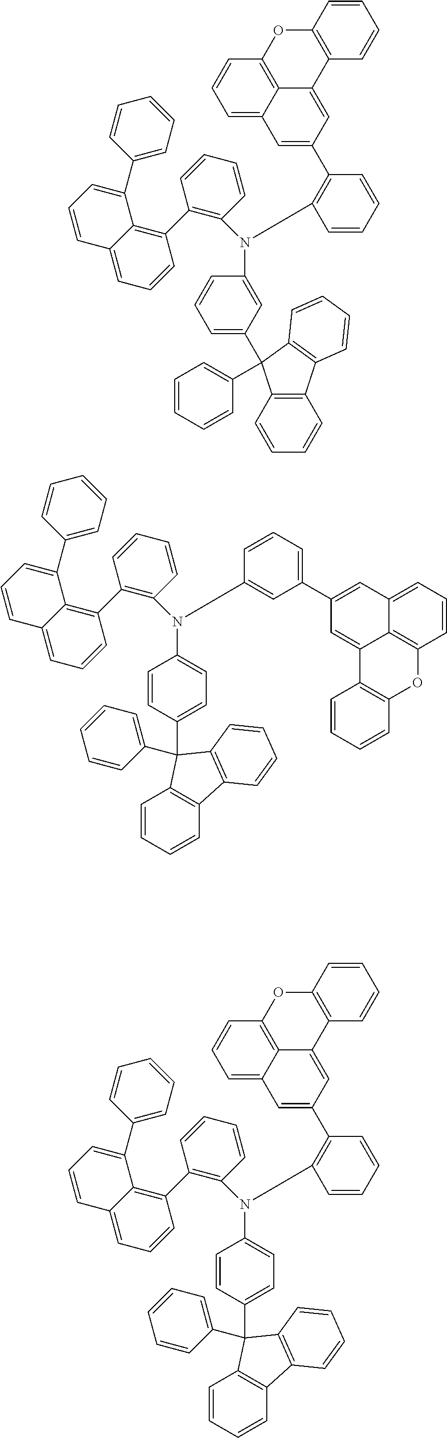 [SMILES: c1ccc(-c2cccc3cccc(-c4ccccc4N(c4cccc(C5(c6ccccc6)c6ccccc6-c6ccccc65)c4)c4ccccc4-c4cc5c6c(cccc6c4)Oc4ccccc4-5)c23)cc1 ; c1ccc(-c2cccc3cccc(-c4ccccc4N(c4ccc(C5(c6ccccc6)c6ccccc6-c6ccccc65)cc4)c4cccc(-c5cc6c7c(cccc7c5)Oc5ccccc5-6)c4)c23)cc1 ; c1ccc(-c2cccc3cccc(-c4ccccc4N(c4ccc(C5(c6ccccc6)c6ccccc6-c6ccccc65)cc4)c4ccccc4-c4cc5c6c(cccc6c4)Oc4ccccc4-5)c23)cc1]

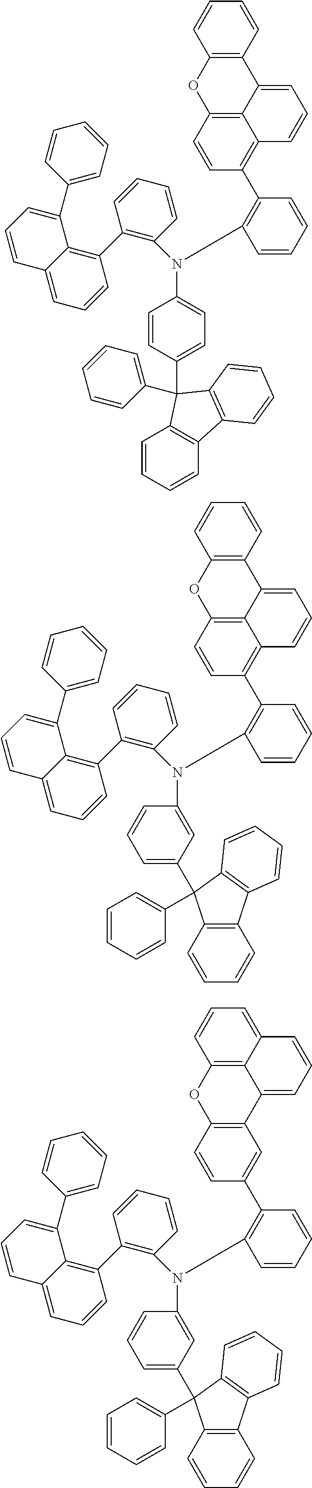

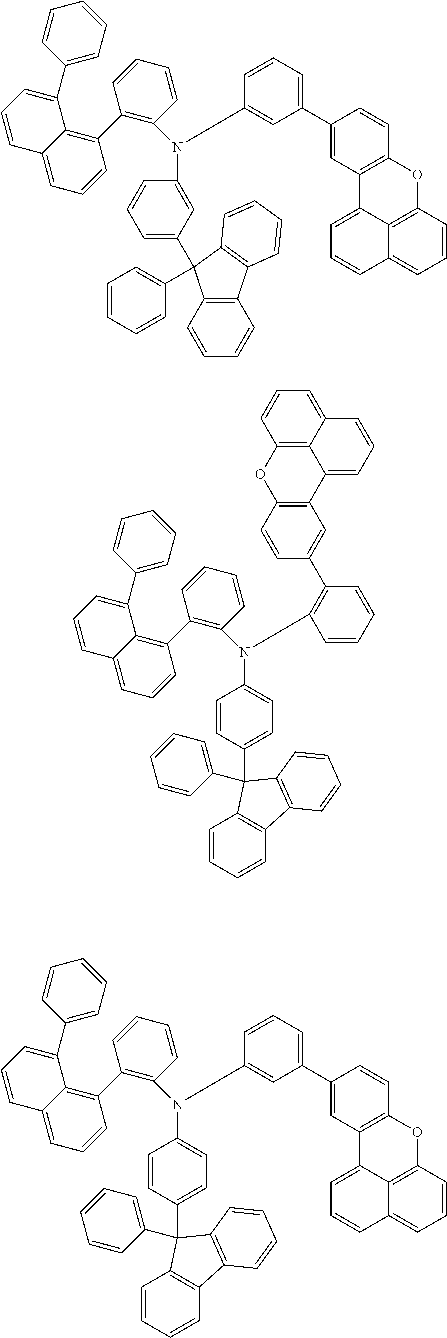 [SMILES: c1ccc(-c2cccc3cccc(-c4ccccc4N(c4cccc(-c5ccc6c(c5)-c5cccc7cccc(c57)O6)c4)c4cccc(C5(c6ccccc6)c6ccccc6-c6ccccc65)c4)c23)cc1 ; c1ccc(-c2cccc3cccc(-c4ccccc4N(c4ccc(C5(c6ccccc6)c6ccccc6-c6ccccc65)cc4)c4ccccc4-c4ccc5c(c4)-c4cccc6cccc(c46)O5)c23)cc1 ; c1ccc(-c2cccc3cccc(-c4ccccc4N(c4ccc(C5(c6ccccc6)c6ccccc6-c6ccccc65)cc4)c4cccc(-c5ccc6c(c5)-c5cccc7cccc(c57)O6)c4)c23)cc1]

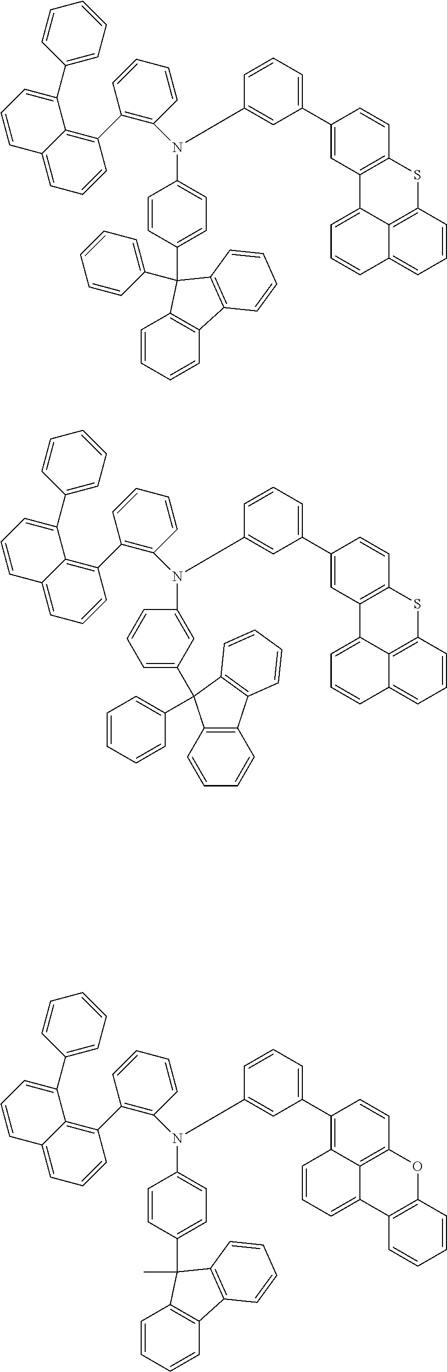 [SMILES: c1ccc(-c2cccc3cccc(-c4ccccc4N(c4ccc(C5(c6ccccc6)c6ccccc6-c6ccccc65)cc4)c4cccc(-c5ccc6c(c5)-c5cccc7cccc(c57)S6)c4)c23)cc1 ; c1ccc(-c2cccc3cccc(-c4ccccc4N(c4cccc(-c5ccc6c(c5)-c5cccc7cccc(c57)S6)c4)c4cccc(C5(c6ccccc6)c6ccccc6-c6ccccc65)c4)c23)cc1 ; CC1(c2ccc(N(c3cccc(-c4ccc5c6c(cccc46)-c4ccccc4O5)c3)c3ccccc3-c3cccc4cccc(-c5ccccc5)c34)cc2)c2ccccc2-c2ccccc21]

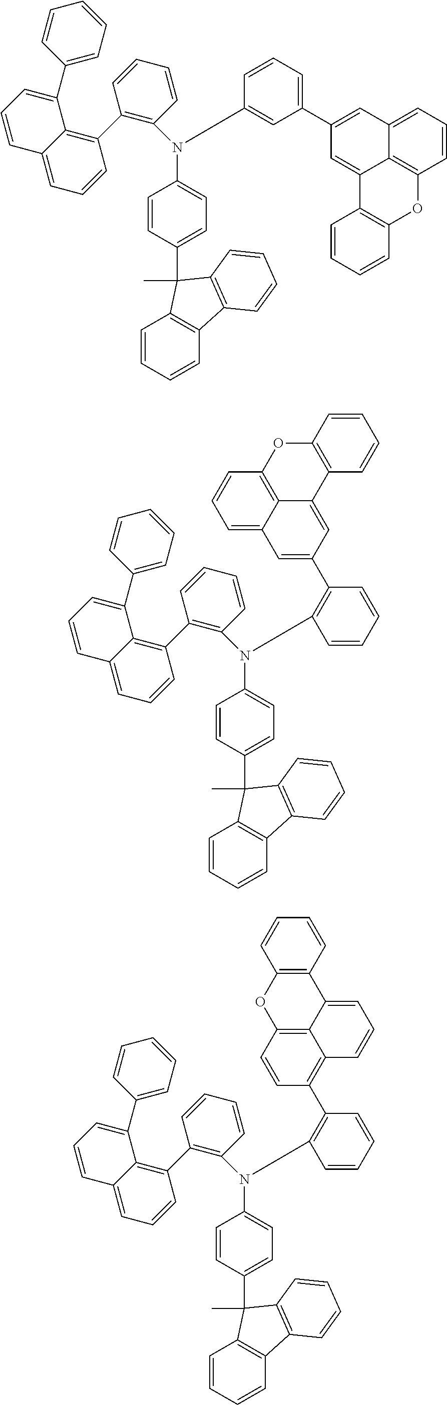 [SMILES: CC1(c2ccc(N(c3cccc(-c4cc5c6c(cccc6c4)Oc4ccccc4-5)c3)c3ccccc3-c3cccc4cccc(-c5ccccc5)c34)cc2)c2ccccc2-c2ccccc21 ; CC1(c2ccc(N(c3ccccc3-c3cc4c5c(cccc5c3)Oc3ccccc3-4)c3ccccc3-c3cccc4cccc(-c5ccccc5)c34)cc2)c2ccccc2-c2ccccc21 ; CC1(c2ccc(N(c3ccccc3-c3ccc4c5c(cccc35)-c3ccccc3O4)c3ccccc3-c3cccc4cccc(-c5ccccc5)c34)cc2)c2ccccc2-c2ccccc21]

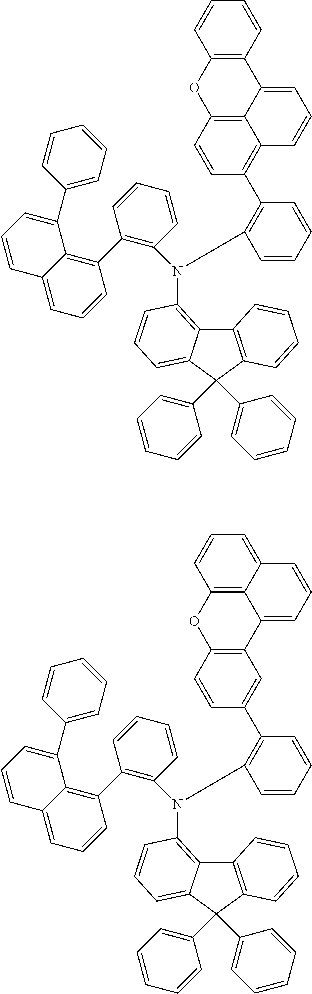 [SMILES: c1ccc(-c2cccc3cccc(-c4ccccc4N(c4ccccc4-c4ccc5c6c(cccc46)-c4ccccc4O5)c4cccc5c4-c4ccccc4C5(c4ccccc4)c4ccccc4)c23)cc1 ; c1ccc(-c2cccc3cccc(-c4ccccc4N(c4ccccc4-c4ccc5c(c4)-c4cccc6cccc(c46)O5)c4cccc5c4-c4ccccc4C5(c4ccccc4)c4ccccc4)c23)cc1]

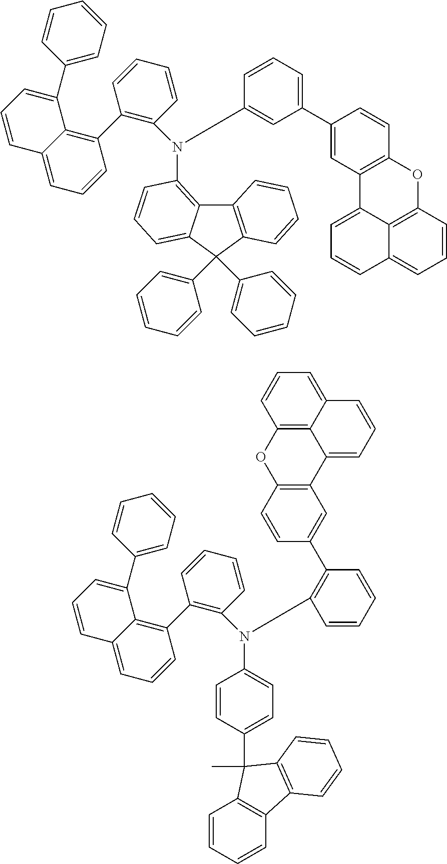

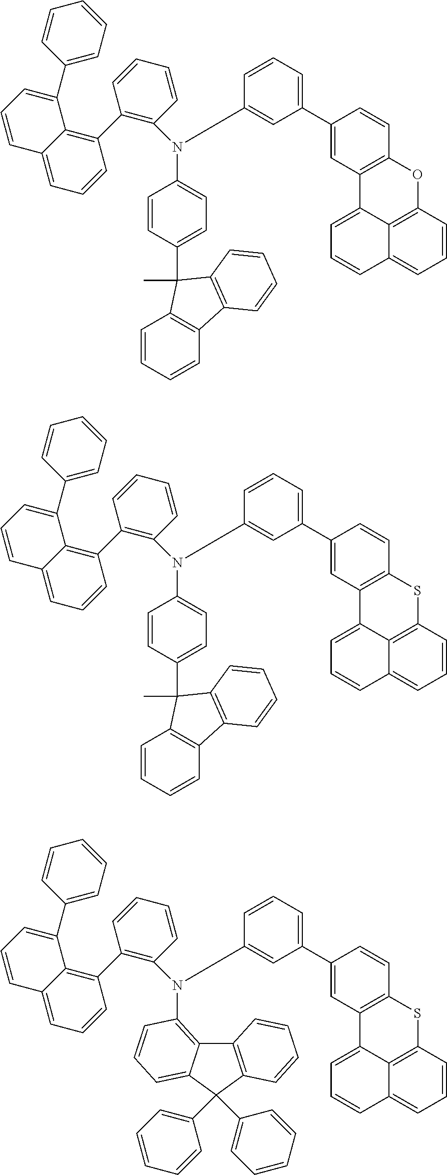 [SMILES: CC1(c2ccc(N(c3cccc(-c4ccc5c(c4)-c4cccc6cccc(c46)O5)c3)c3ccccc3-c3cccc4cccc(-c5ccccc5)c34)cc2)c2ccccc2-c2ccccc21 ; CC1(c2ccc(N(c3cccc(-c4ccc5c(c4)-c4cccc6cccc(c46)S5)c3)c3ccccc3-c3cccc4cccc(-c5ccccc5)c34)cc2)c2ccccc2-c2ccccc21 ; c1ccc(-c2cccc3cccc(-c4ccccc4N(c4cccc(-c5ccc6c(c5)-c5cccc7cccc(c57)S6)c4)c4cccc5c4-c4ccccc4C5(c4ccccc4)c4ccccc4)c23)cc1]

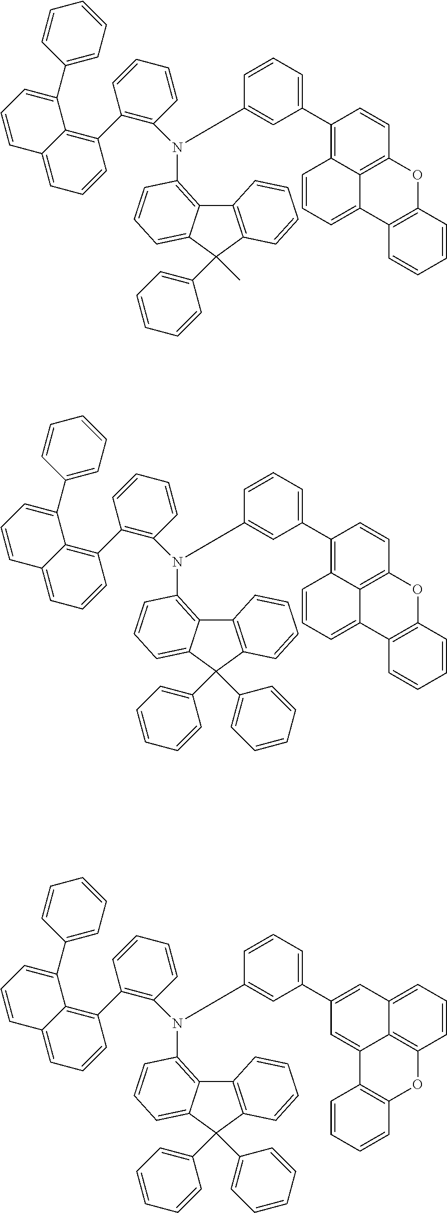 [SMILES: CC1(c2ccccc2)c2ccccc2-c2c(N(c3cccc(-c4ccc5c6c(cccc46)-c4ccccc4O5)c3)c3ccccc3-c3cccc4cccc(-c5ccccc5)c34)cccc21 ; c1ccc(-c2cccc3cccc(-c4ccccc4N(c4cccc(-c5ccc6c7c(cccc57)-c5ccccc5O6)c4)c4cccc5c4-c4ccccc4C5(c4ccccc4)c4ccccc4)c23)cc1 ; c1ccc(-c2cccc3cccc(-c4ccccc4N(c4cccc(-c5cc6c7c(cccc7c5)Oc5ccccc5-6)c4)c4cccc5c4-c4ccccc4C5(c4ccccc4)c4ccccc4)c23)cc1]

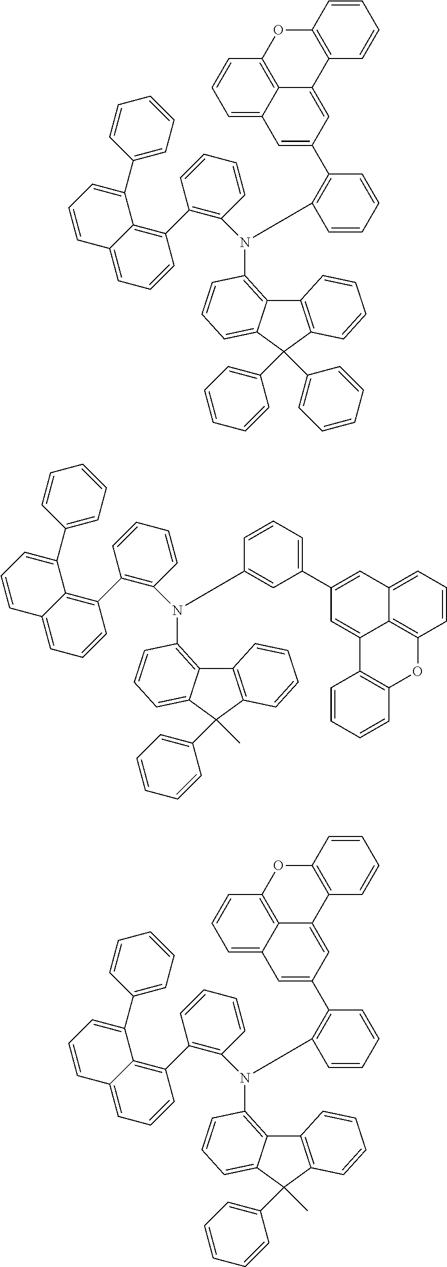 [SMILES: c1ccc(-c2cccc3cccc(-c4ccccc4N(c4ccccc4-c4cc5c6c(cccc6c4)Oc4ccccc4-5)c4cccc5c4-c4ccccc4C5(c4ccccc4)c4ccccc4)c23)cc1 ; CC1(c2ccccc2)c2ccccc2-c2c(N(c3cccc(-c4cc5c6c(cccc6c4)Oc4ccccc4-5)c3)c3ccccc3-c3cccc4cccc(-c5ccccc5)c34)cccc21 ; CC1(c2ccccc2)c2ccccc2-c2c(N(c3ccccc3-c3cc4c5c(cccc5c3)Oc3ccccc3-4)c3ccccc3-c3cccc4cccc(-c5ccccc5)c34)cccc21]

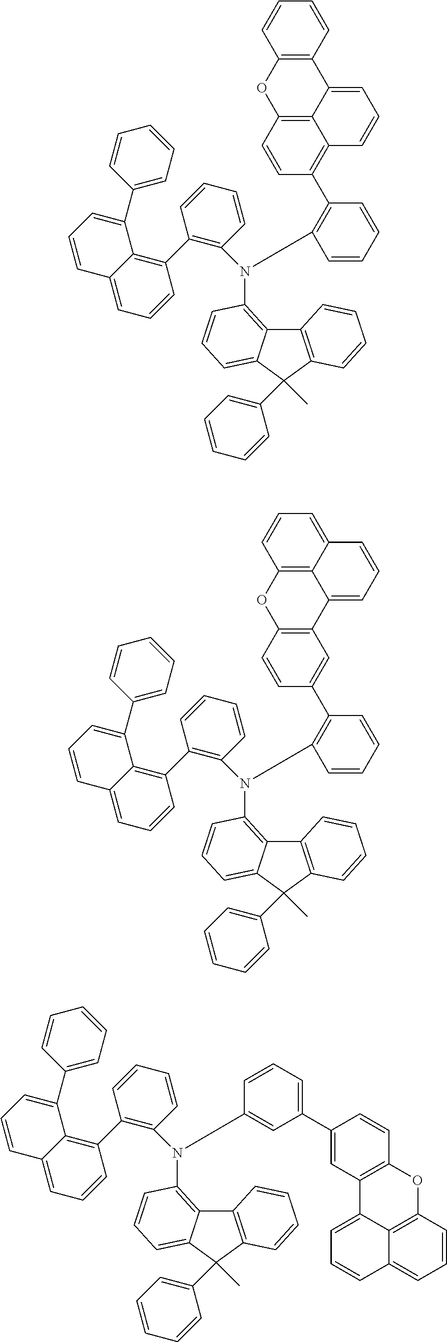 [SMILES: CC1(c2ccccc2)c2ccccc2-c2c(N(c3ccccc3-c3ccc4c5c(cccc35)-c3ccccc3O4)c3ccccc3-c3cccc4cccc(-c5ccccc5)c34)cccc21 ; CC1(c2ccccc2)c2ccccc2-c2c(N(c3ccccc3-c3ccc4c(c3)-c3cccc5cccc(c35)O4)c3ccccc3-c3cccc4cccc(-c5ccccc5)c34)cccc21 ; CC1(c2ccccc2)c2ccccc2-c2c(N(c3cccc(-c4ccc5c(c4)-c4cccc6cccc(c46)O5)c3)c3ccccc3-c3cccc4cccc(-c5ccccc5)c34)cccc21]

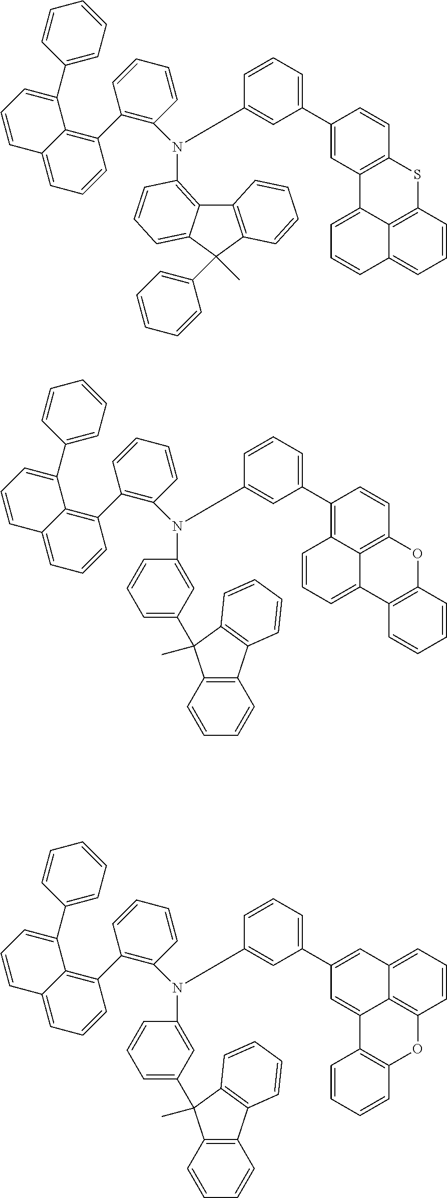 [SMILES: CC1(c2ccccc2)c2ccccc2-c2c(N(c3cccc(-c4ccc5c(c4)-c4cccc6cccc(c46)S5)c3)c3ccccc3-c3cccc4cccc(-c5ccccc5)c34)cccc21 ; CC1(c2cccc(N(c3cccc(-c4ccc5c6c(cccc46)-c4ccccc4O5)c3)c3ccccc3-c3cccc4cccc(-c5ccccc5)c34)c2)c2ccccc2-c2ccccc21 ; CC1(c2cccc(N(c3cccc(-c4cc5c6c(cccc6c4)Oc4ccccc4-5)c3)c3ccccc3-c3cccc4cccc(-c5ccccc5)c34)c2)c2ccccc2-c2ccccc21]

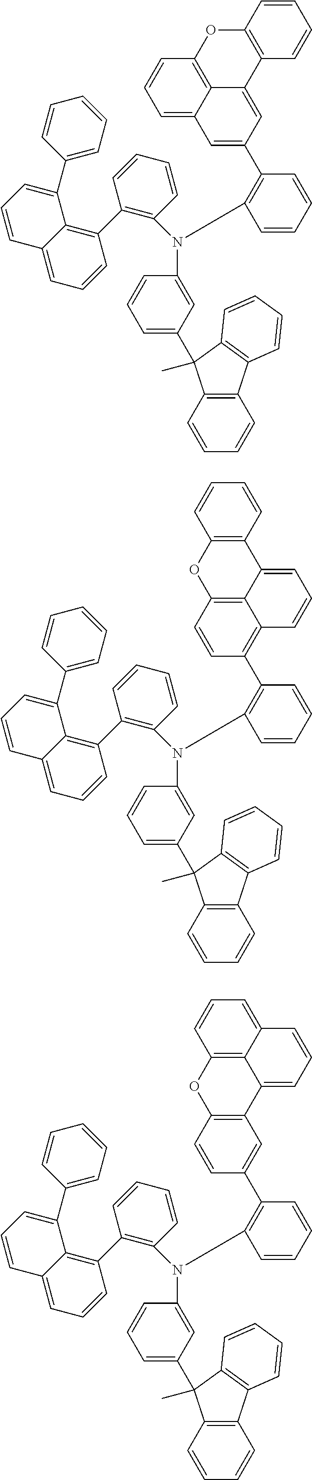

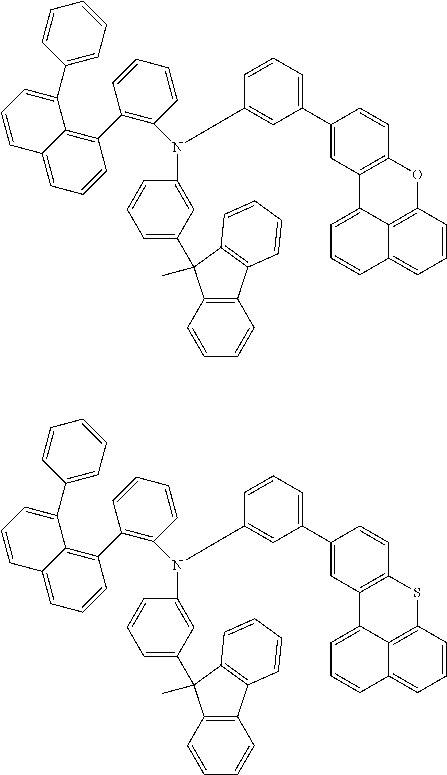 [SMILES: CC1(c2cccc(N(c3cccc(-c4ccc5c(c4)-c4cccc6cccc(c46)O5)c3)c3ccccc3-c3cccc4cccc(-c5ccccc5)c34)c2)c2ccccc2-c2ccccc21 ; CC1(c2cccc(N(c3cccc(-c4ccc5c(c4)-c4cccc6cccc(c46)S5)c3)c3ccccc3-c3cccc4cccc(-c5ccccc5)c34)c2)c2ccccc2-c2ccccc21]

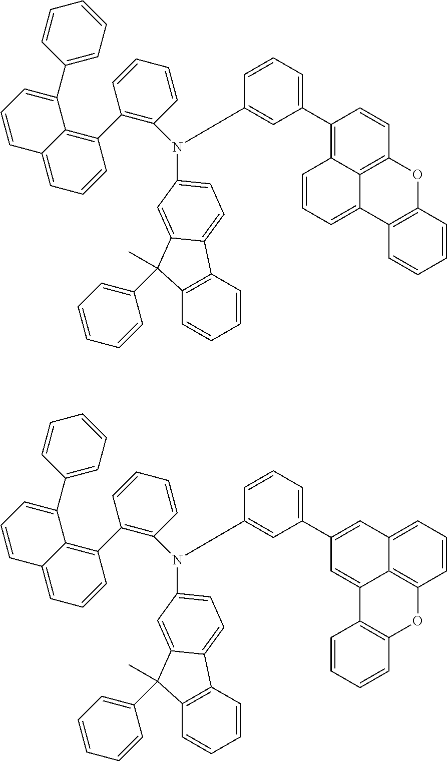 [SMILES: CC1(c2ccccc2)c2ccccc2-c2ccc(N(c3cccc(-c4ccc5c6c(cccc46)-c4ccccc4O5)c3)c3ccccc3-c3cccc4cccc(-c5ccccc5)c34)cc21 ; CC1(c2ccccc2)c2ccccc2-c2ccc(N(c3cccc(-c4cc5c6c(cccc6c4)Oc4ccccc4-5)c3)c3ccccc3-c3cccc4cccc(-c5ccccc5)c34)cc21]

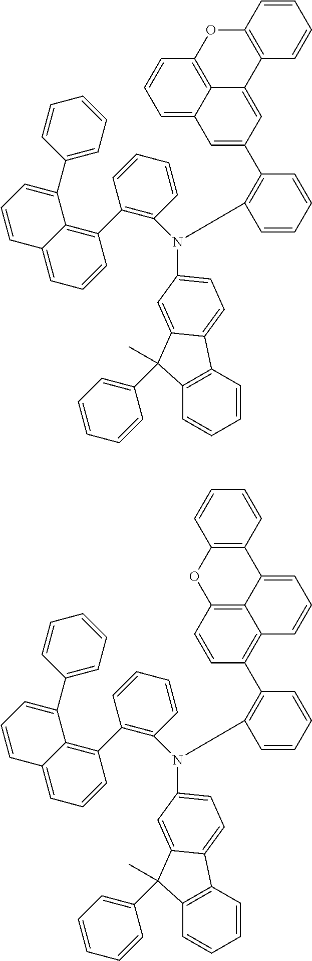 [SMILES: CC1(c2ccccc2)c2ccccc2-c2ccc(N(c3ccccc3-c3cc4c5c(cccc5c3)Oc3ccccc3-4)c3ccccc3-c3cccc4cccc(-c5ccccc5)c34)cc21 ; CC1(c2ccccc2)c2ccccc2-c2ccc(N(c3ccccc3-c3ccc4c5c(cccc35)-c3ccccc3O4)c3ccccc3-c3cccc4cccc(-c5ccccc5)c34)cc21]

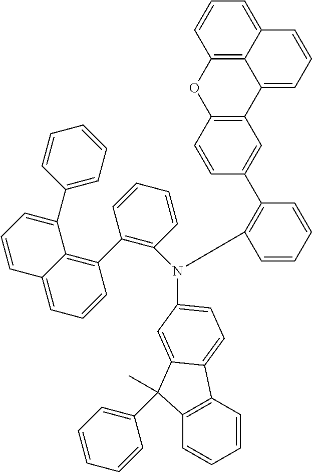 [SMILES: CC1(c2ccccc2)c2ccccc2-c2ccc(N(c3ccccc3-c3ccc4c(c3)-c3cccc5cccc(c35)O4)c3ccccc3-c3cccc4cccc(-c5ccccc5)c34)cc21]

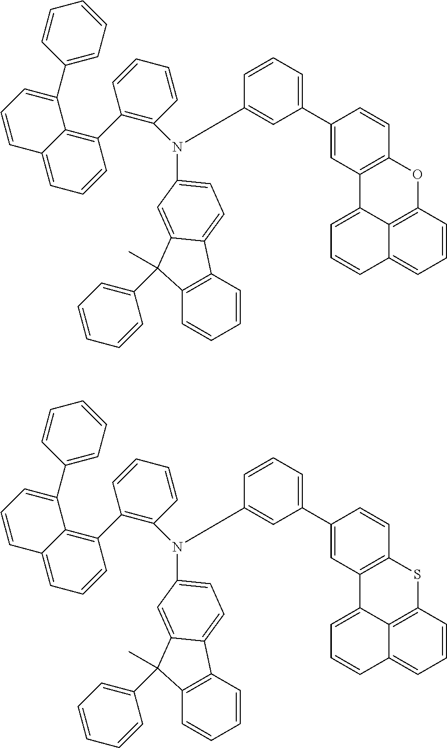 [SMILES: CC1(c2ccccc2)c2ccccc2-c2ccc(N(c3cccc(-c4ccc5c(c4)-c4cccc6cccc(c46)O5)c3)c3ccccc3-c3cccc4cccc(-c5ccccc5)c34)cc21 ; CC1(c2ccccc2)c2ccccc2-c2ccc(N(c3cccc(-c4ccc5c(c4)-c4cccc6cccc(c46)S5)c3)c3ccccc3-c3cccc4cccc(-c5ccccc5)c34)cc21]

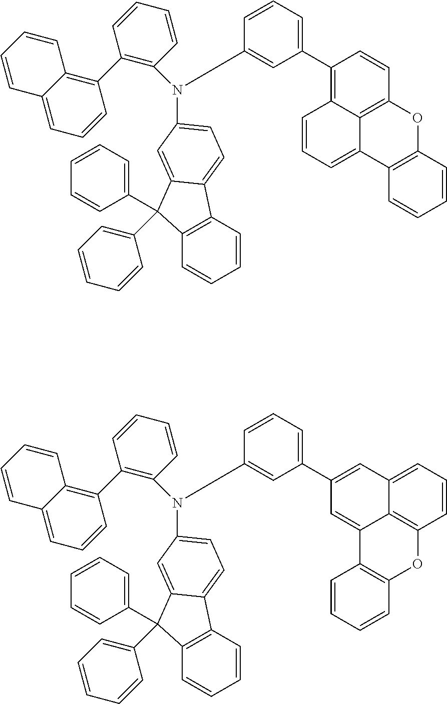 [SMILES: c1ccc(C2(c3ccccc3)c3ccccc3-c3ccc(N(c4cccc(-c5ccc6c7c(cccc57)-c5ccccc5O6)c4)c4ccccc4-c4cccc5ccccc45)cc32)cc1 ; c1ccc(C2(c3ccccc3)c3ccccc3-c3ccc(N(c4cccc(-c5cc6c7c(cccc7c5)Oc5ccccc5-6)c4)c4ccccc4-c4cccc5ccccc45)cc32)cc1]

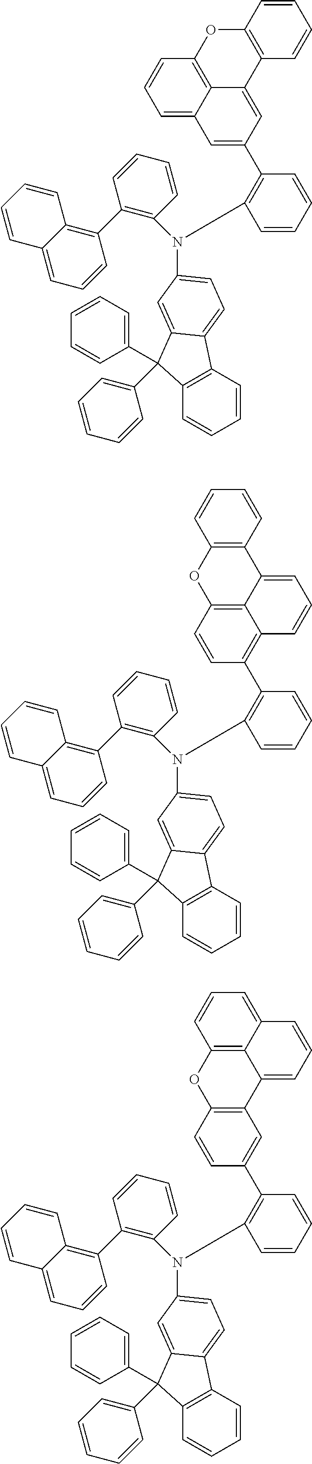

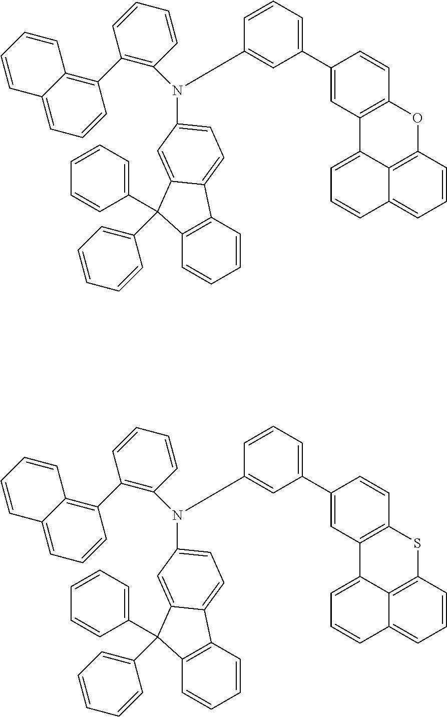 [SMILES: c1ccc(C2(c3ccccc3)c3ccccc3-c3ccc(N(c4cccc(-c5ccc6c(c5)-c5cccc7cccc(c57)O6)c4)c4ccccc4-c4cccc5ccccc45)cc32)cc1 ; c1ccc(C2(c3ccccc3)c3ccccc3-c3ccc(N(c4cccc(-c5ccc6c(c5)-c5cccc7cccc(c57)S6)c4)c4ccccc4-c4cccc5ccccc45)cc32)cc1]

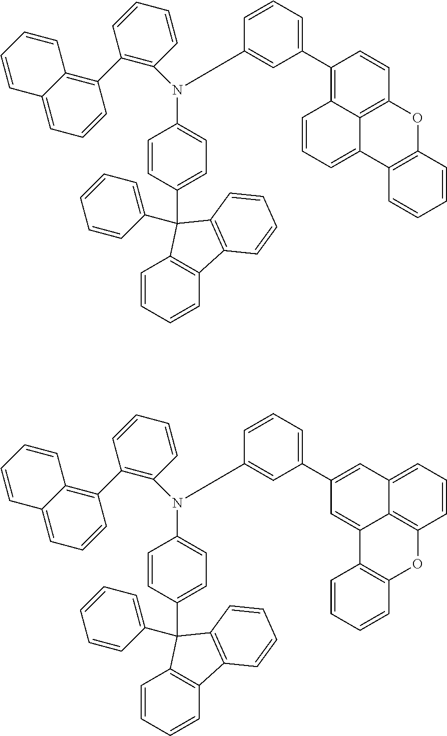 [SMILES: c1ccc(C2(c3ccc(N(c4cccc(-c5ccc6c7c(cccc57)-c5ccccc5O6)c4)c4ccccc4-c4cccc5ccccc45)cc3)c3ccccc3-c3ccccc32)cc1 ; c1ccc(C2(c3ccc(N(c4cccc(-c5cc6c7c(cccc7c5)Oc5ccccc5-6)c4)c4ccccc4-c4cccc5ccccc45)cc3)c3ccccc3-c3ccccc32)cc1]

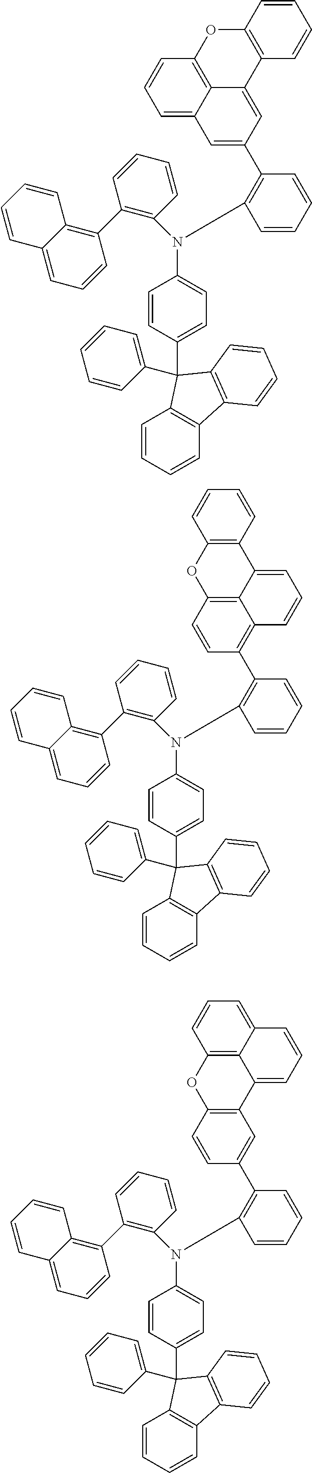

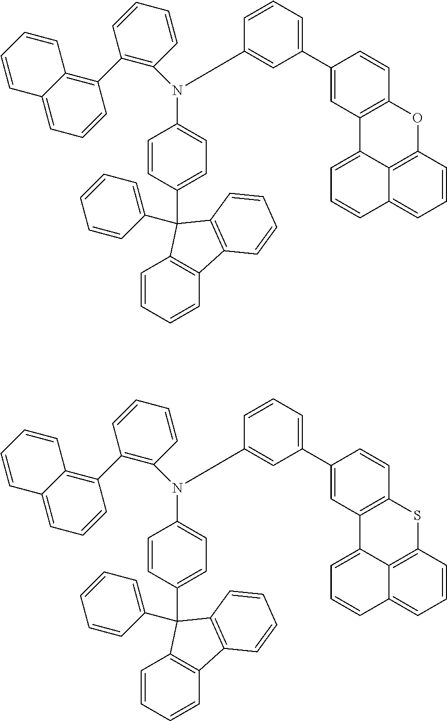 [SMILES: c1ccc(C2(c3ccc(N(c4cccc(-c5ccc6c(c5)-c5cccc7cccc(c57)O6)c4)c4ccccc4-c4cccc5ccccc45)cc3)c3ccccc3-c3ccccc32)cc1 ; c1ccc(C2(c3ccc(N(c4cccc(-c5ccc6c(c5)-c5cccc7cccc(c57)S6)c4)c4ccccc4-c4cccc5ccccc45)cc3)c3ccccc3-c3ccccc32)cc1]

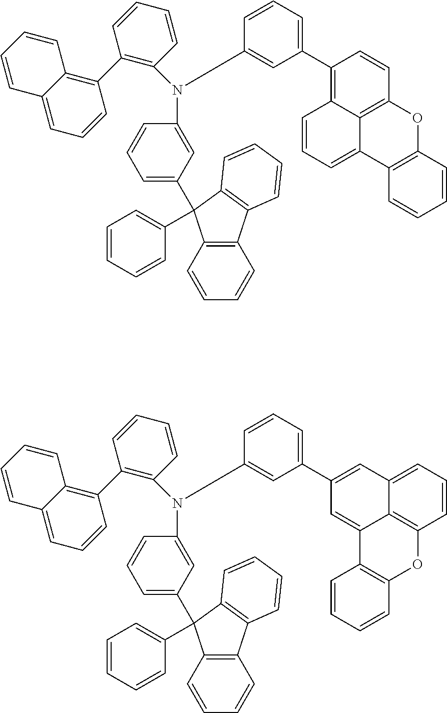 [SMILES: c1ccc(C2(c3cccc(N(c4cccc(-c5ccc6c7c(cccc57)-c5ccccc5O6)c4)c4ccccc4-c4cccc5ccccc45)c3)c3ccccc3-c3ccccc32)cc1 ; c1ccc(C2(c3cccc(N(c4cccc(-c5cc6c7c(cccc7c5)Oc5ccccc5-6)c4)c4ccccc4-c4cccc5ccccc45)c3)c3ccccc3-c3ccccc32)cc1]

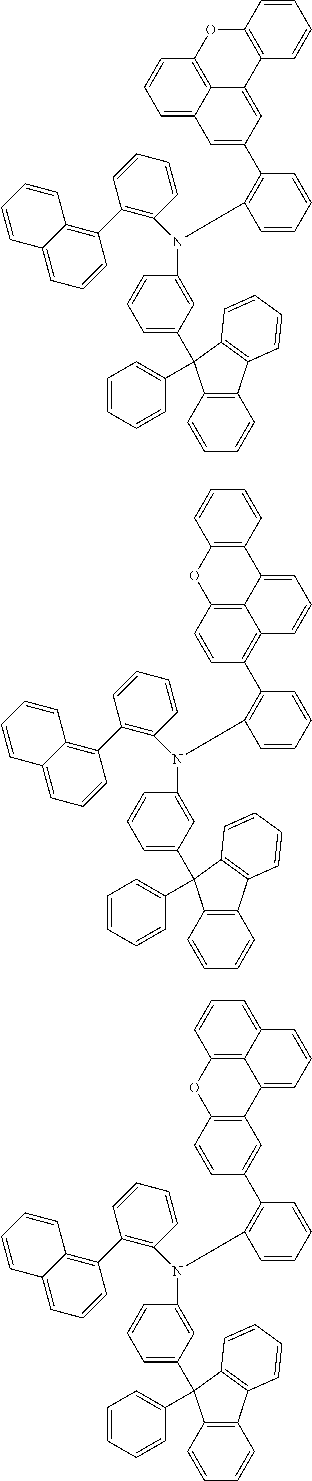

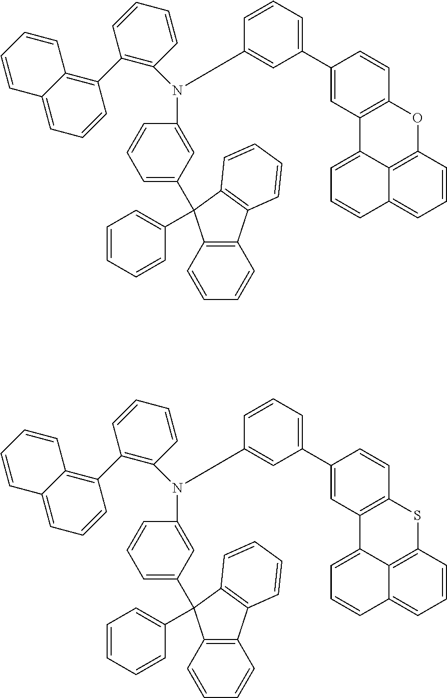 [SMILES: c1ccc(C2(c3cccc(N(c4cccc(-c5ccc6c(c5)-c5cccc7cccc(c57)O6)c4)c4ccccc4-c4cccc5ccccc45)c3)c3ccccc3-c3ccccc32)cc1 ; c1ccc(C2(c3cccc(N(c4cccc(-c5ccc6c(c5)-c5cccc7cccc(c57)S6)c4)c4ccccc4-c4cccc5ccccc45)c3)c3ccccc3-c3ccccc32)cc1]

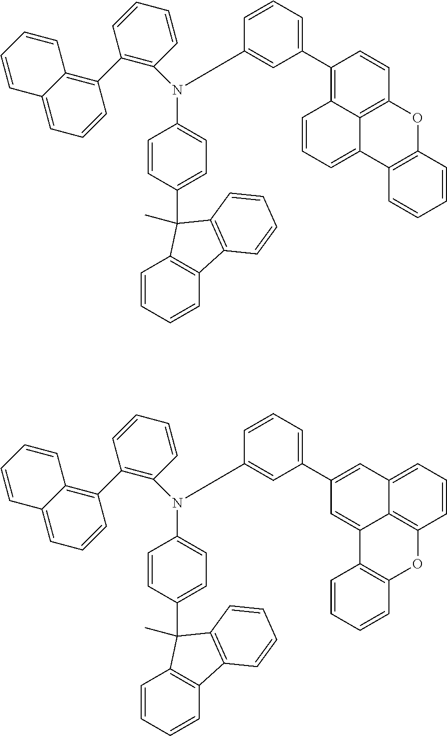 [SMILES: CC1(c2ccc(N(c3cccc(-c4ccc5c6c(cccc46)-c4ccccc4O5)c3)c3ccccc3-c3cccc4ccccc34)cc2)c2ccccc2-c2ccccc21 ; CC1(c2ccc(N(c3cccc(-c4cc5c6c(cccc6c4)Oc4ccccc4-5)c3)c3ccccc3-c3cccc4ccccc34)cc2)c2ccccc2-c2ccccc21]

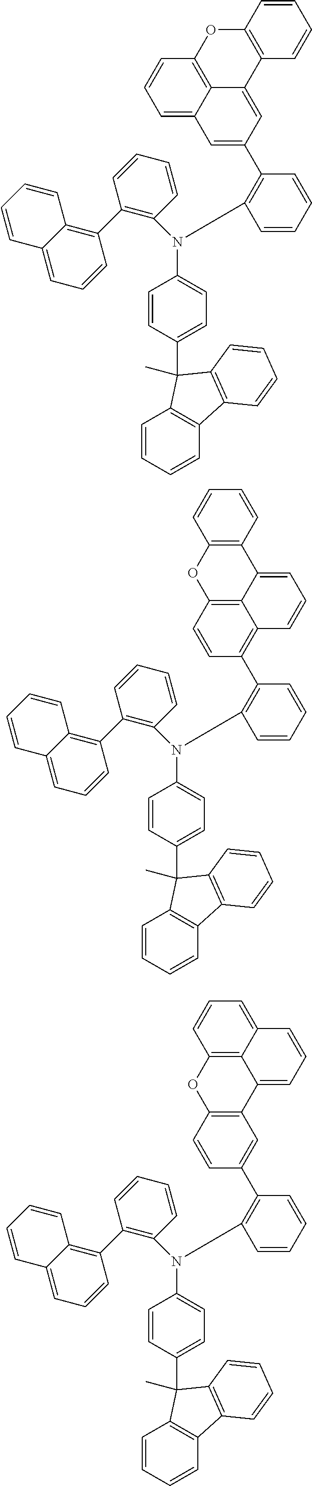

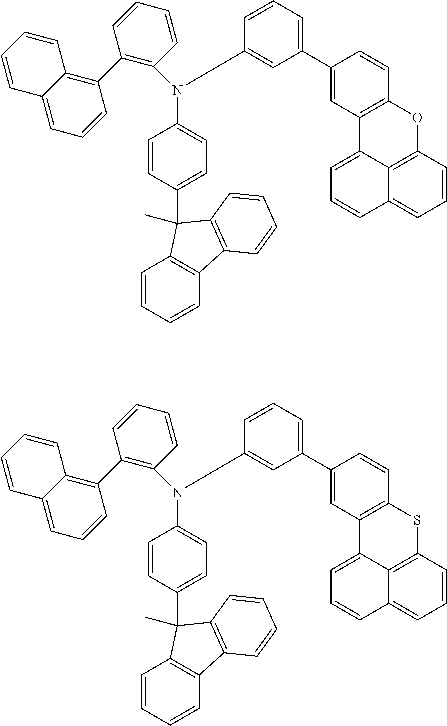 [SMILES: CC1(c2ccc(N(c3cccc(-c4ccc5c(c4)-c4cccc6cccc(c46)O5)c3)c3ccccc3-c3cccc4ccccc34)cc2)c2ccccc2-c2ccccc21 ; CC1(c2ccc(N(c3cccc(-c4ccc5c(c4)-c4cccc6cccc(c46)S5)c3)c3ccccc3-c3cccc4ccccc34)cc2)c2ccccc2-c2ccccc21]

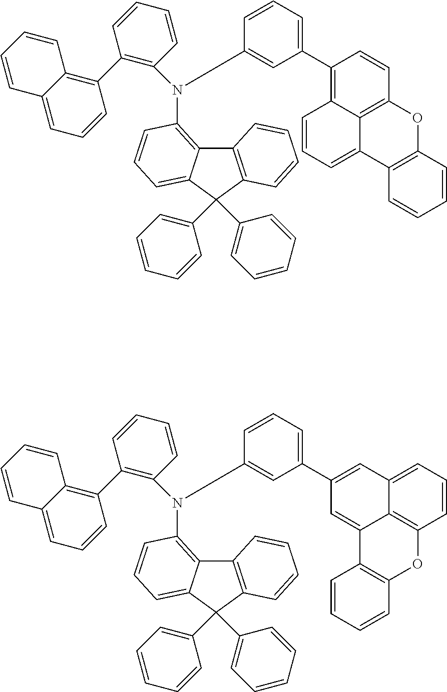 [SMILES: c1ccc(C2(c3ccccc3)c3ccccc3-c3c(N(c4cccc(-c5ccc6c7c(cccc57)-c5ccccc5O6)c4)c4ccccc4-c4cccc5ccccc45)cccc32)cc1 ; c1ccc(C2(c3ccccc3)c3ccccc3-c3c(N(c4cccc(-c5cc6c7c(cccc7c5)Oc5ccccc5-6)c4)c4ccccc4-c4cccc5ccccc45)cccc32)cc1]

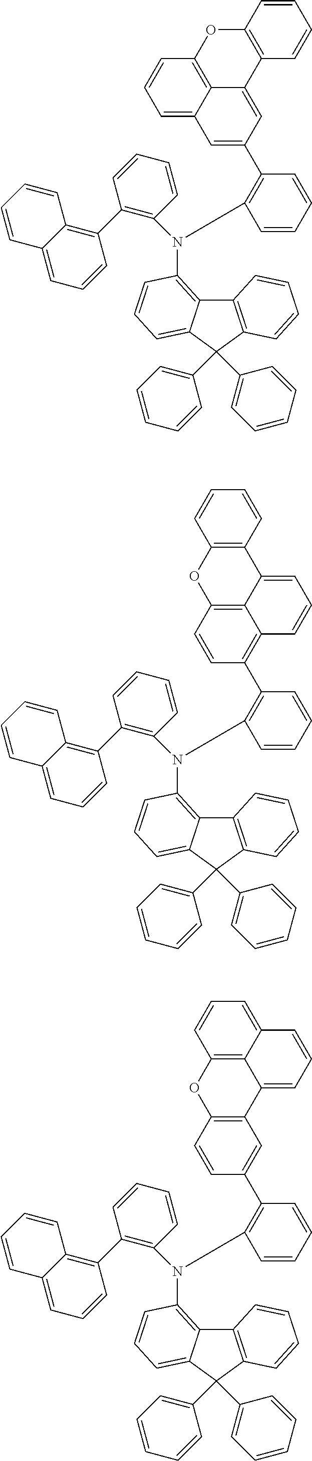 [SMILES: c1ccc(C2(c3ccccc3)c3ccccc3-c3c(N(c4ccccc4-c4cc5c6c(cccc6c4)Oc4ccccc4-5)c4ccccc4-c4cccc5ccccc45)cccc32)cc1 ; c1ccc(C2(c3ccccc3)c3ccccc3-c3c(N(c4ccccc4-c4cccc5ccccc45)c4ccccc4-c4ccc5c6c(cccc46)-c4ccccc4O5)cccc32)cc1 ; c1ccc(C2(c3ccccc3)c3ccccc3-c3c(N(c4ccccc4-c4ccc5c(c4)-c4cccc6cccc(c46)O5)c4ccccc4-c4cccc5ccccc45)cccc32)cc1]

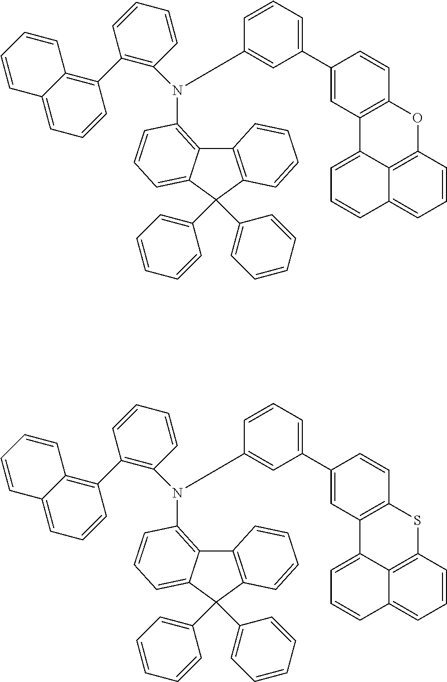 [SMILES: c1ccc(C2(c3ccccc3)c3ccccc3-c3c(N(c4cccc(-c5ccc6c(c5)-c5cccc7cccc(c57)O6)c4)c4ccccc4-c4cccc5ccccc45)cccc32)cc1 ; c1ccc(C2(c3ccccc3)c3ccccc3-c3c(N(c4cccc(-c5ccc6c(c5)-c5cccc7cccc(c57)S6)c4)c4ccccc4-c4cccc5ccccc45)cccc32)cc1]

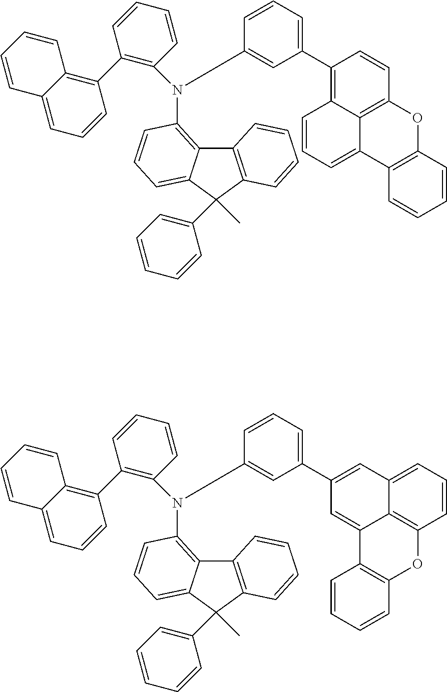 [SMILES: CC1(c2ccccc2)c2ccccc2-c2c(N(c3cccc(-c4ccc5c6c(cccc46)-c4ccccc4O5)c3)c3ccccc3-c3cccc4ccccc34)cccc21 ; CC1(c2ccccc2)c2ccccc2-c2c(N(c3cccc(-c4cc5c6c(cccc6c4)Oc4ccccc4-5)c3)c3ccccc3-c3cccc4ccccc34)cccc21]

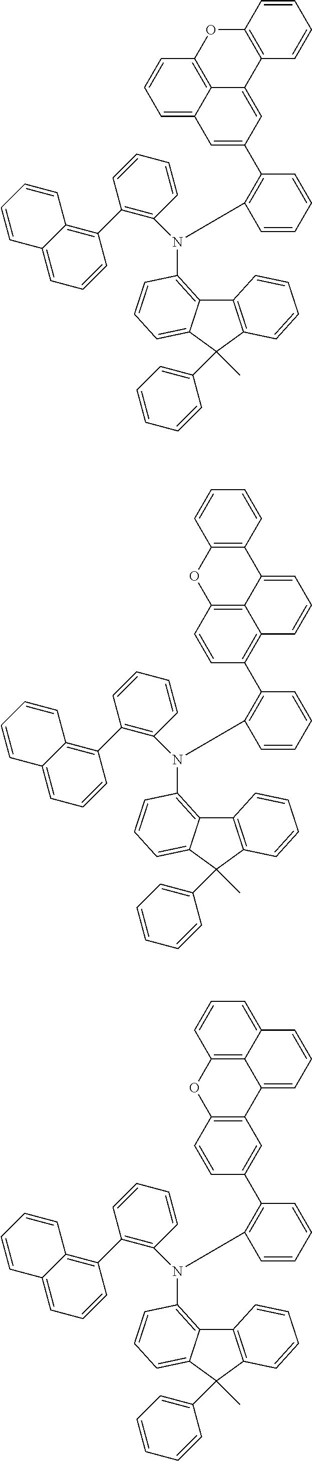 [SMILES: CC1(c2ccccc2)c2ccccc2-c2c(N(c3ccccc3-c3cc4c5c(cccc5c3)Oc3ccccc3-4)c3ccccc3-c3cccc4ccccc34)cccc21 ; CC1(c2ccccc2)c2ccccc2-c2c(N(c3ccccc3-c3cccc4ccccc34)c3ccccc3-c3ccc4c5c(cccc35)-c3ccccc3O4)cccc21 ; CC1(c2ccccc2)c2ccccc2-c2c(N(c3ccccc3-c3ccc4c(c3)-c3cccc5cccc(c35)O4)c3ccccc3-c3cccc4ccccc34)cccc21]

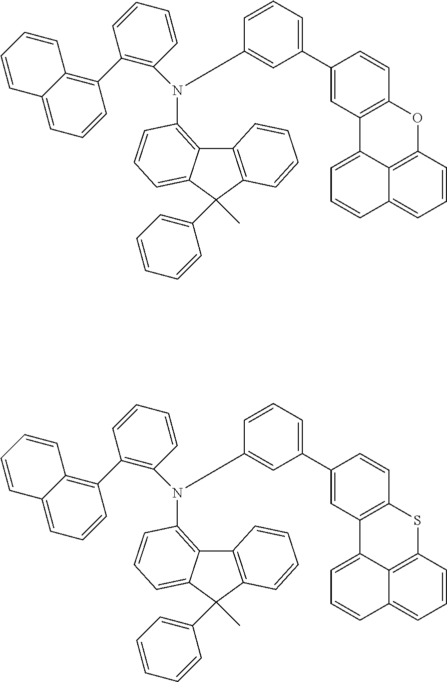 [SMILES: CC1(c2ccccc2)c2ccccc2-c2c(N(c3cccc(-c4ccc5c(c4)-c4cccc6cccc(c46)O5)c3)c3ccccc3-c3cccc4ccccc34)cccc21 ; CC1(c2ccccc2)c2ccccc2-c2c(N(c3cccc(-c4ccc5c(c4)-c4cccc6cccc(c46)S5)c3)c3ccccc3-c3cccc4ccccc34)cccc21]

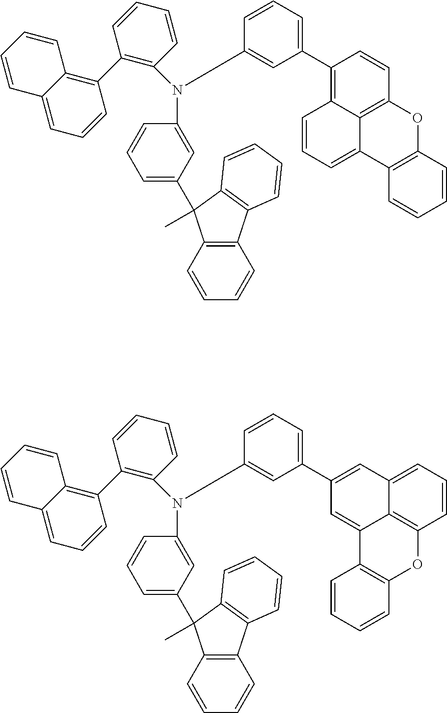 [SMILES: CC1(c2cccc(N(c3cccc(-c4ccc5c6c(cccc46)-c4ccccc4O5)c3)c3ccccc3-c3cccc4ccccc34)c2)c2ccccc2-c2ccccc21 ; CC1(c2cccc(N(c3cccc(-c4cc5c6c(cccc6c4)Oc4ccccc4-5)c3)c3ccccc3-c3cccc4ccccc34)c2)c2ccccc2-c2ccccc21]

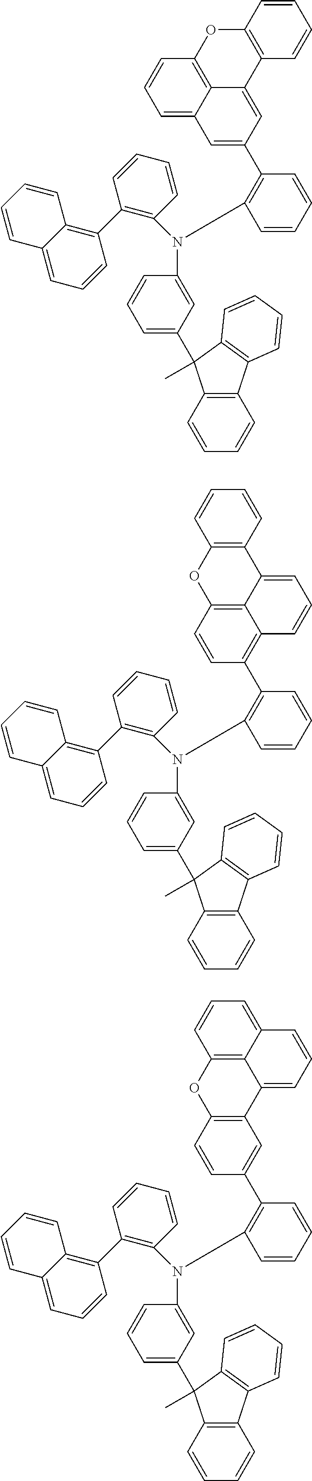

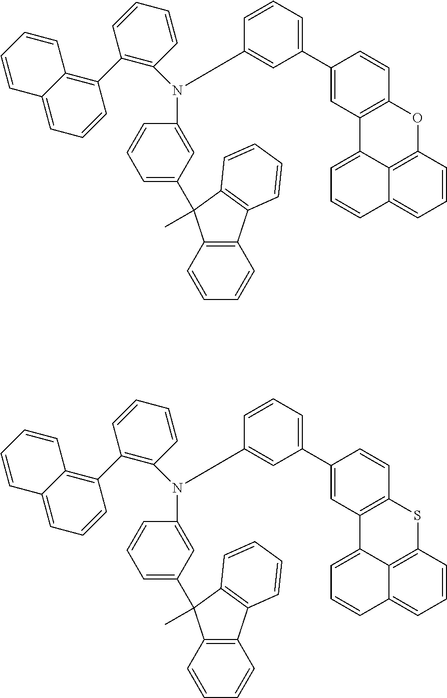 [SMILES: CC1(c2cccc(N(c3cccc(-c4ccc5c(c4)-c4cccc6cccc(c46)O5)c3)c3ccccc3-c3cccc4ccccc34)c2)c2ccccc2-c2ccccc21 ; CC1(c2cccc(N(c3cccc(-c4ccc5c(c4)-c4cccc6cccc(c46)S5)c3)c3ccccc3-c3cccc4ccccc34)c2)c2ccccc2-c2ccccc21]

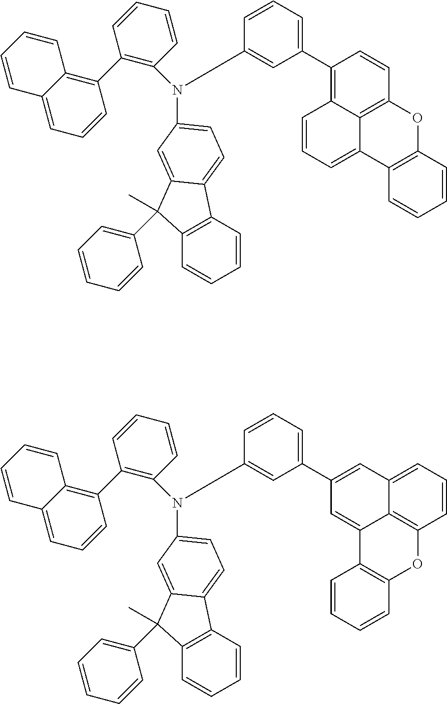 [SMILES: CC1(c2ccccc2)c2ccccc2-c2ccc(N(c3cccc(-c4ccc5c6c(cccc46)-c4ccccc4O5)c3)c3ccccc3-c3cccc4ccccc34)cc21 ; CC1(c2ccccc2)c2ccccc2-c2ccc(N(c3cccc(-c4cc5c6c(cccc6c4)Oc4ccccc4-5)c3)c3ccccc3-c3cccc4ccccc34)cc21]

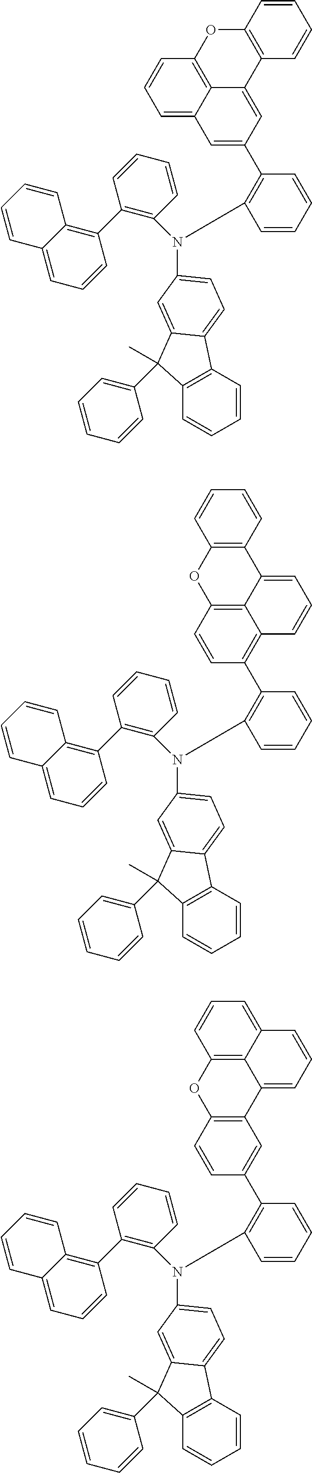 [SMILES: CC1(c2ccccc2)c2ccccc2-c2ccc(N(c3ccccc3-c3cc4c5c(cccc5c3)Oc3ccccc3-4)c3ccccc3-c3cccc4ccccc34)cc21 ; CC1(c2ccccc2)c2ccccc2-c2ccc(N(c3ccccc3-c3cccc4ccccc34)c3ccccc3-c3ccc4c5c(cccc35)-c3ccccc3O4)cc21 ; CC1(c2ccccc2)c2ccccc2-c2ccc(N(c3ccccc3-c3ccc4c(c3)-c3cccc5cccc(c35)O4)c3ccccc3-c3cccc4ccccc34)cc21]

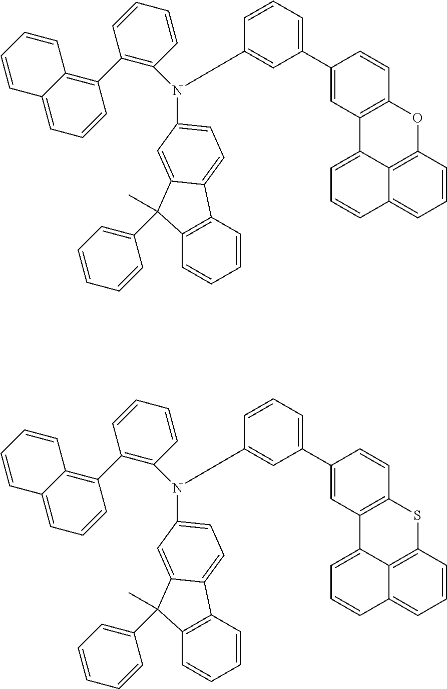 [SMILES: CC1(c2ccccc2)c2ccccc2-c2ccc(N(c3cccc(-c4ccc5c(c4)-c4cccc6cccc(c46)O5)c3)c3ccccc3-c3cccc4ccccc34)cc21 ; CC1(c2ccccc2)c2ccccc2-c2ccc(N(c3cccc(-c4ccc5c(c4)-c4cccc6cccc(c46)S5)c3)c3ccccc3-c3cccc4ccccc34)cc21]

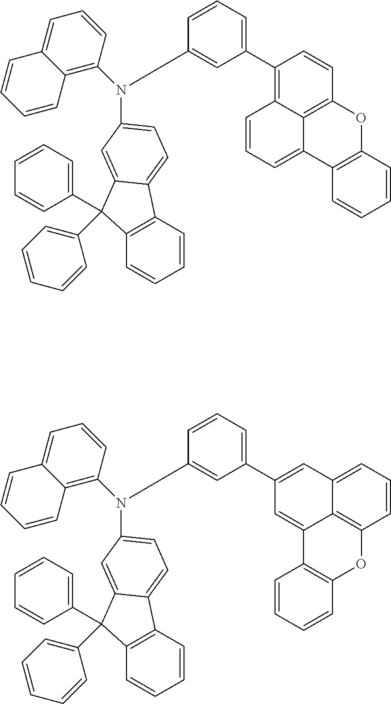 [SMILES: c1ccc(C2(c3ccccc3)c3ccccc3-c3ccc(N(c4cccc(-c5ccc6c7c(cccc57)-c5ccccc5O6)c4)c4cccc5ccccc45)cc32)cc1 ; c1ccc(C2(c3ccccc3)c3ccccc3-c3ccc(N(c4cccc(-c5cc6c7c(cccc7c5)Oc5ccccc5-6)c4)c4cccc5ccccc45)cc32)cc1]

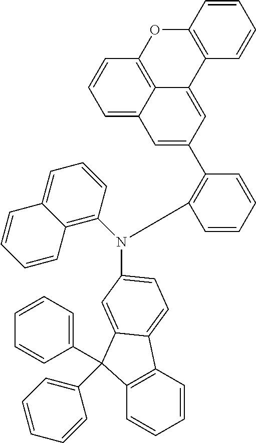 [SMILES: c1ccc(C2(c3ccccc3)c3ccccc3-c3ccc(N(c4ccccc4-c4cc5c6c(cccc6c4)Oc4ccccc4-5)c4cccc5ccccc45)cc32)cc1]

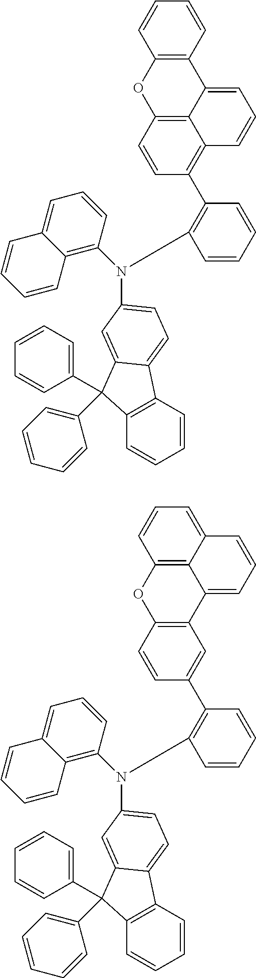

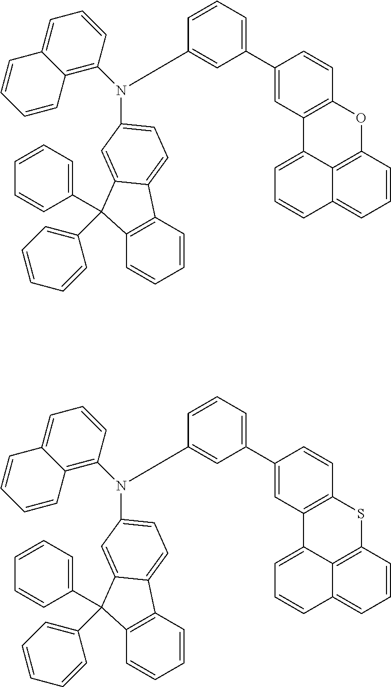 [SMILES: c1ccc(C2(c3ccccc3)c3ccccc3-c3ccc(N(c4cccc(-c5ccc6c(c5)-c5cccc7cccc(c57)O6)c4)c4cccc5ccccc45)cc32)cc1 ; c1ccc(C2(c3ccccc3)c3ccccc3-c3ccc(N(c4cccc(-c5ccc6c(c5)-c5cccc7cccc(c57)S6)c4)c4cccc5ccccc45)cc32)cc1]

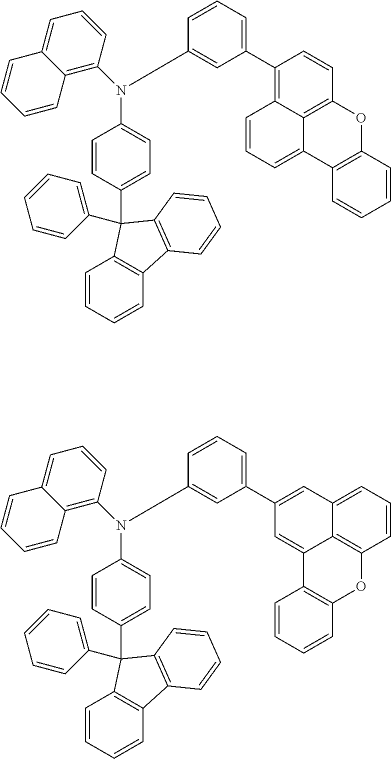 [SMILES: c1ccc(C2(c3ccc(N(c4cccc(-c5ccc6c7c(cccc57)-c5ccccc5O6)c4)c4cccc5ccccc45)cc3)c3ccccc3-c3ccccc32)cc1 ; c1ccc(C2(c3ccc(N(c4cccc(-c5cc6c7c(cccc7c5)Oc5ccccc5-6)c4)c4cccc5ccccc45)cc3)c3ccccc3-c3ccccc32)cc1]

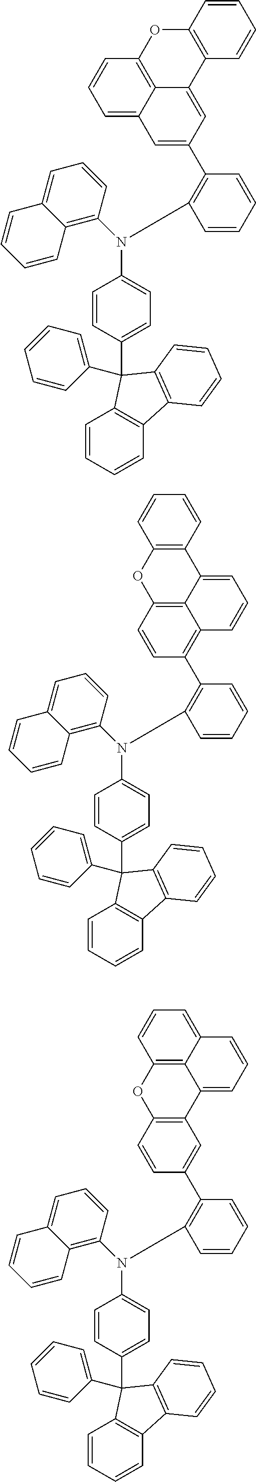

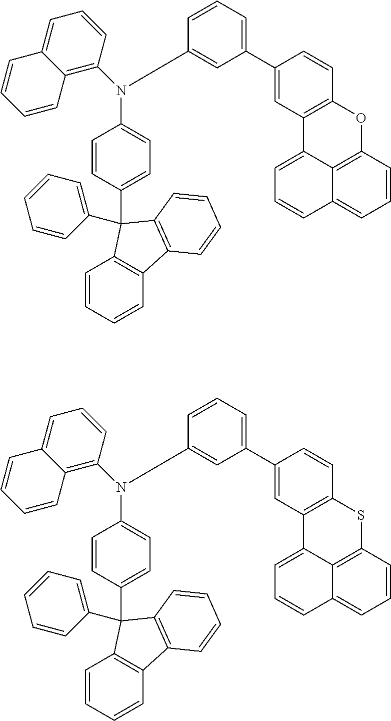 [SMILES: c1ccc(C2(c3ccc(N(c4cccc(-c5ccc6c(c5)-c5cccc7cccc(c57)O6)c4)c4cccc5ccccc45)cc3)c3ccccc3-c3ccccc32)cc1 ; c1ccc(C2(c3ccc(N(c4cccc(-c5ccc6c(c5)-c5cccc7cccc(c57)S6)c4)c4cccc5ccccc45)cc3)c3ccccc3-c3ccccc32)cc1]

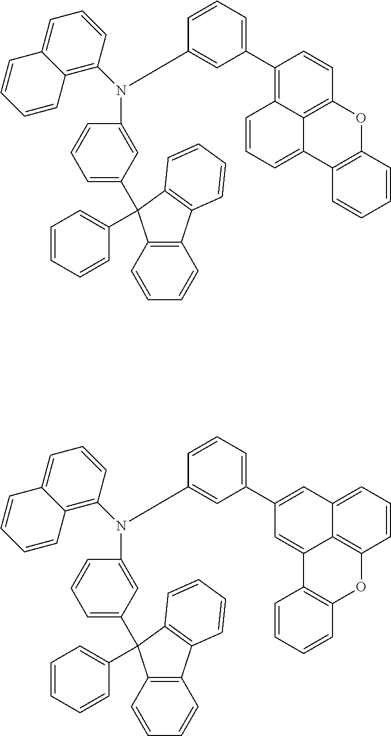 [SMILES: c1ccc(C2(c3cccc(N(c4cccc(-c5ccc6c7c(cccc57)-c5ccccc5O6)c4)c4cccc5ccccc45)c3)c3ccccc3-c3ccccc32)cc1 ; c1ccc(C2(c3cccc(N(c4cccc(-c5cc6c7c(cccc7c5)Oc5ccccc5-6)c4)c4cccc5ccccc45)c3)c3ccccc3-c3ccccc32)cc1]

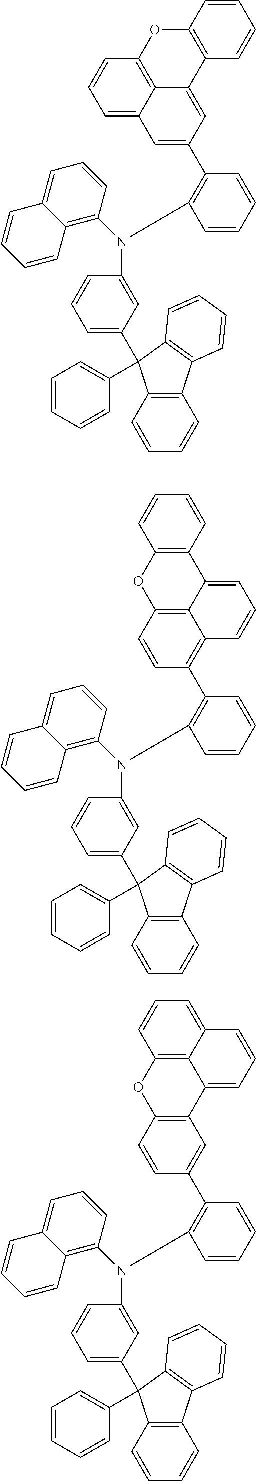

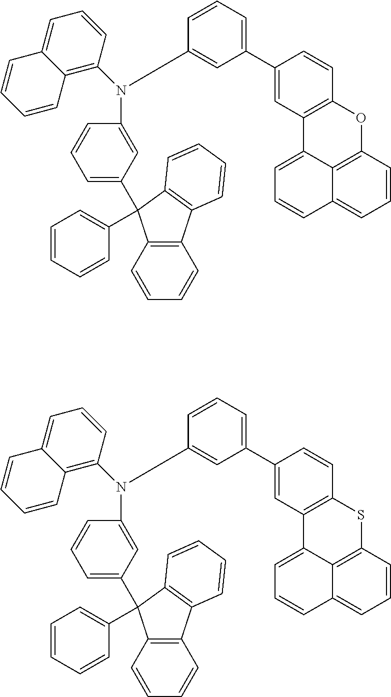 [SMILES: c1ccc(C2(c3cccc(N(c4cccc(-c5ccc6c(c5)-c5cccc7cccc(c57)O6)c4)c4cccc5ccccc45)c3)c3ccccc3-c3ccccc32)cc1 ; c1ccc(C2(c3cccc(N(c4cccc(-c5ccc6c(c5)-c5cccc7cccc(c57)S6)c4)c4cccc5ccccc45)c3)c3ccccc3-c3ccccc32)cc1]

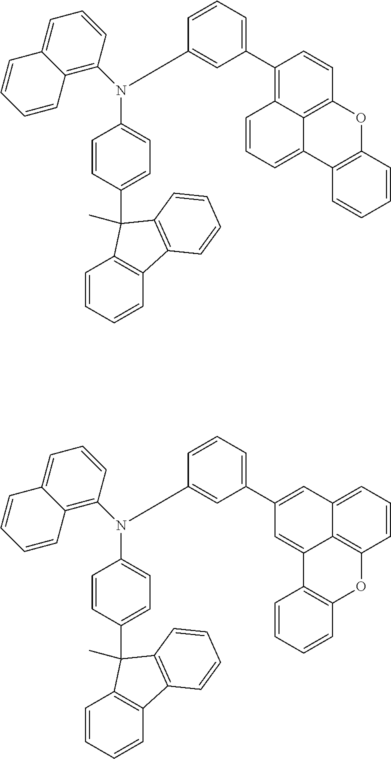 [SMILES: CC1(c2ccc(N(c3cccc(-c4ccc5c6c(cccc46)-c4ccccc4O5)c3)c3cccc4ccccc34)cc2)c2ccccc2-c2ccccc21 ; CC1(c2ccc(N(c3cccc(-c4cc5c6c(cccc6c4)Oc4ccccc4-5)c3)c3cccc4ccccc34)cc2)c2ccccc2-c2ccccc21]

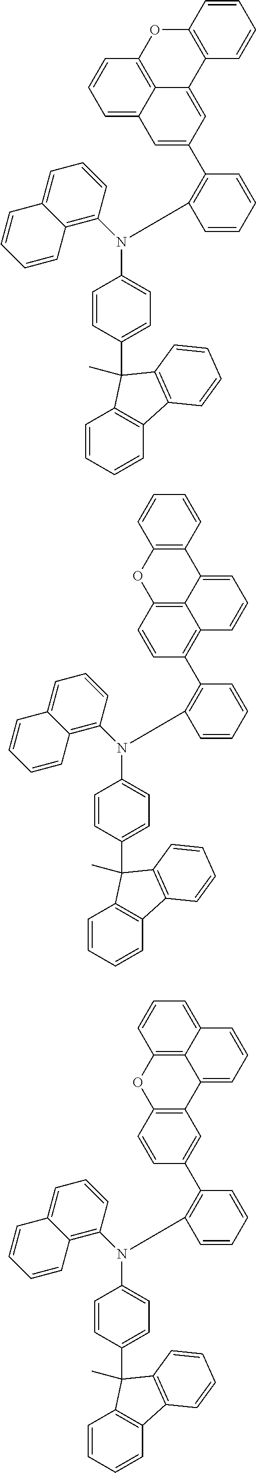

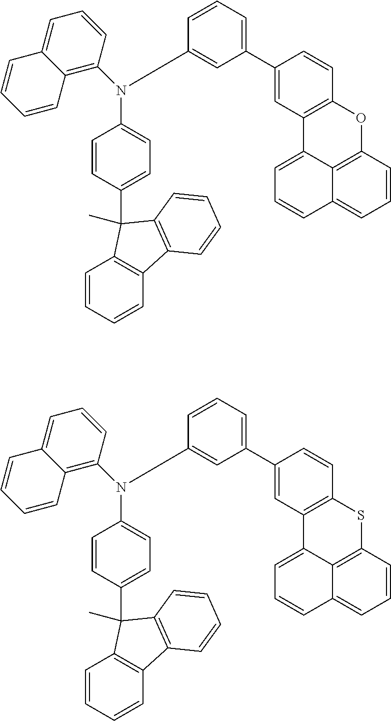 [SMILES: CC1(c2ccc(N(c3cccc(-c4ccc5c(c4)-c4cccc6cccc(c46)O5)c3)c3cccc4ccccc34)cc2)c2ccccc2-c2ccccc21 ; CC1(c2ccc(N(c3cccc(-c4ccc5c(c4)-c4cccc6cccc(c46)S5)c3)c3cccc4ccccc34)cc2)c2ccccc2-c2ccccc21]

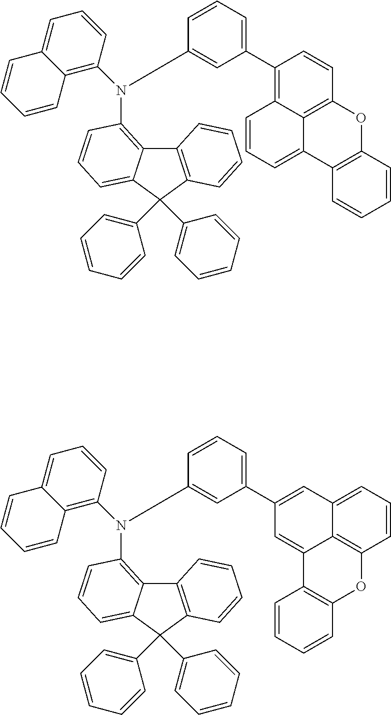 [SMILES: c1ccc(C2(c3ccccc3)c3ccccc3-c3c(N(c4cccc(-c5ccc6c7c(cccc57)-c5ccccc5O6)c4)c4cccc5ccccc45)cccc32)cc1 ; c1ccc(C2(c3ccccc3)c3ccccc3-c3c(N(c4cccc(-c5cc6c7c(cccc7c5)Oc5ccccc5-6)c4)c4cccc5ccccc45)cccc32)cc1]

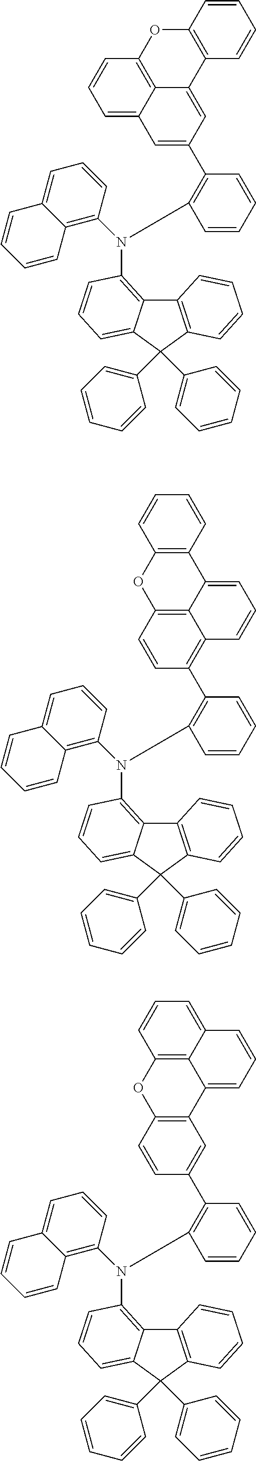 [SMILES: c1ccc(C2(c3ccccc3)c3ccccc3-c3c(N(c4ccccc4-c4cc5c6c(cccc6c4)Oc4ccccc4-5)c4cccc5ccccc45)cccc32)cc1 ; c1ccc(C2(c3ccccc3)c3ccccc3-c3c(N(c4ccccc4-c4ccc5c6c(cccc46)-c4ccccc4O5)c4cccc5ccccc45)cccc32)cc1 ; c1ccc(C2(c3ccccc3)c3ccccc3-c3c(N(c4ccccc4-c4ccc5c(c4)-c4cccc6cccc(c46)O5)c4cccc5ccccc45)cccc32)cc1]

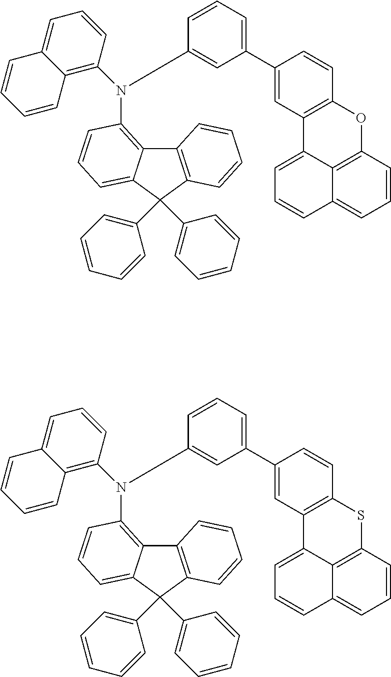 [SMILES: c1ccc(C2(c3ccccc3)c3ccccc3-c3c(N(c4cccc(-c5ccc6c(c5)-c5cccc7cccc(c57)O6)c4)c4cccc5ccccc45)cccc32)cc1 ; c1ccc(C2(c3ccccc3)c3ccccc3-c3c(N(c4cccc(-c5ccc6c(c5)-c5cccc7cccc(c57)S6)c4)c4cccc5ccccc45)cccc32)cc1]

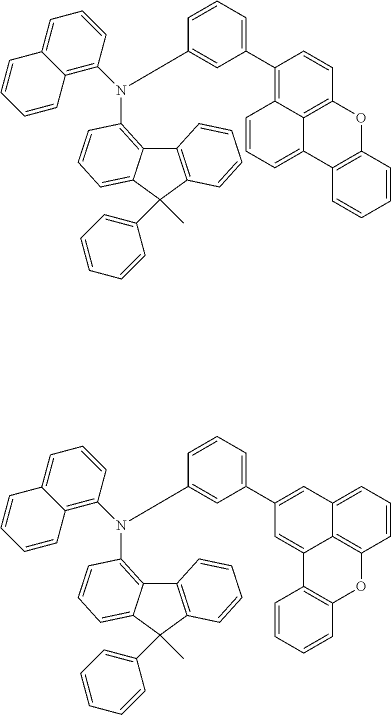 [SMILES: CC1(c2ccccc2)c2ccccc2-c2c(N(c3cccc(-c4ccc5c6c(cccc46)-c4ccccc4O5)c3)c3cccc4ccccc34)cccc21 ; CC1(c2ccccc2)c2ccccc2-c2c(N(c3cccc(-c4cc5c6c(cccc6c4)Oc4ccccc4-5)c3)c3cccc4ccccc34)cccc21]

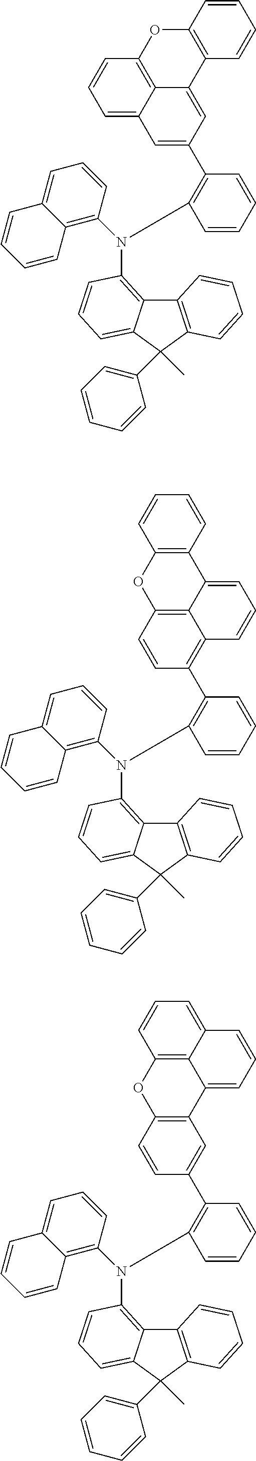 [SMILES: CC1(c2ccccc2)c2ccccc2-c2c(N(c3ccccc3-c3cc4c5c(cccc5c3)Oc3ccccc3-4)c3cccc4ccccc34)cccc21 ; CC1(c2ccccc2)c2ccccc2-c2c(N(c3ccccc3-c3ccc4c5c(cccc35)-c3ccccc3O4)c3cccc4ccccc34)cccc21 ; CC1(c2ccccc2)c2ccccc2-c2c(N(c3ccccc3-c3ccc4c(c3)-c3cccc5cccc(c35)O4)c3cccc4ccccc34)cccc21]

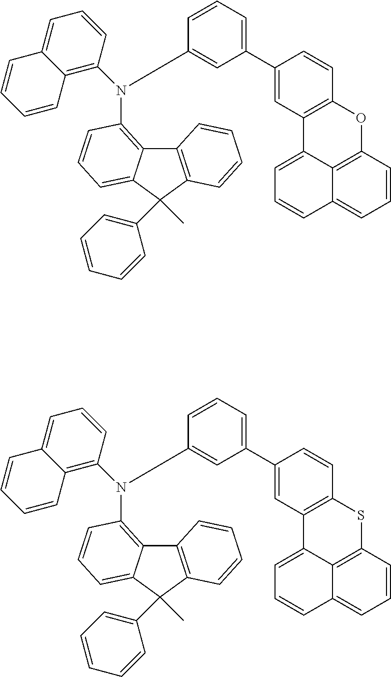 [SMILES: CC1(c2ccccc2)c2ccccc2-c2c(N(c3cccc(-c4ccc5c(c4)-c4cccc6cccc(c46)O5)c3)c3cccc4ccccc34)cccc21 ; CC1(c2ccccc2)c2ccccc2-c2c(N(c3cccc(-c4ccc5c(c4)-c4cccc6cccc(c46)S5)c3)c3cccc4ccccc34)cccc21]

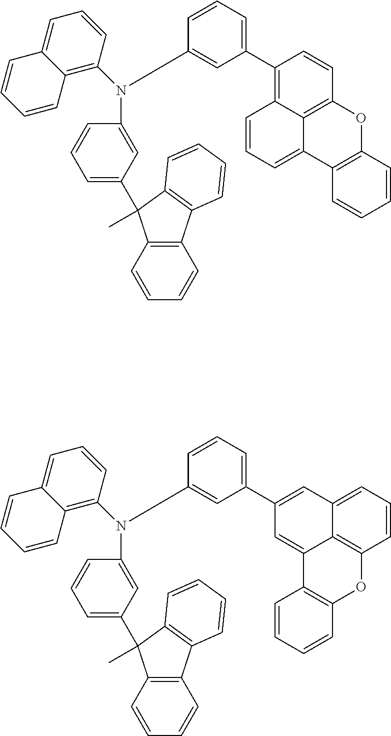 [SMILES: CC1(c2cccc(N(c3cccc(-c4ccc5c6c(cccc46)-c4ccccc4O5)c3)c3cccc4ccccc34)c2)c2ccccc2-c2ccccc21 ; CC1(c2cccc(N(c3cccc(-c4cc5c6c(cccc6c4)Oc4ccccc4-5)c3)c3cccc4ccccc34)c2)c2ccccc2-c2ccccc21]

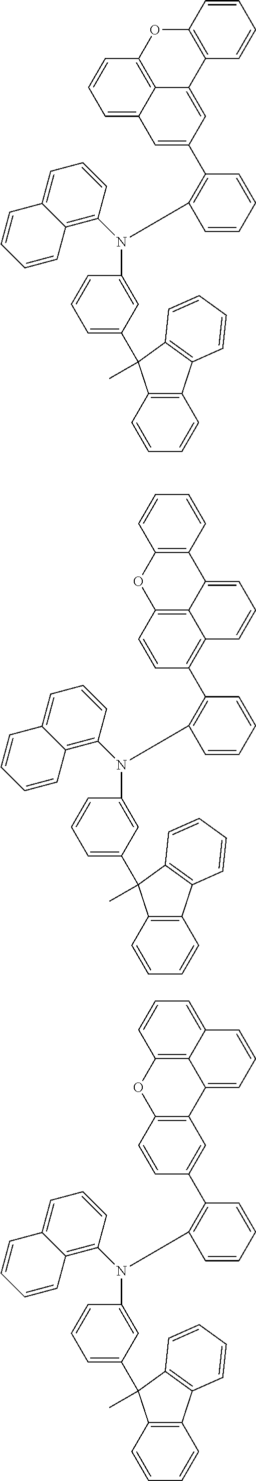

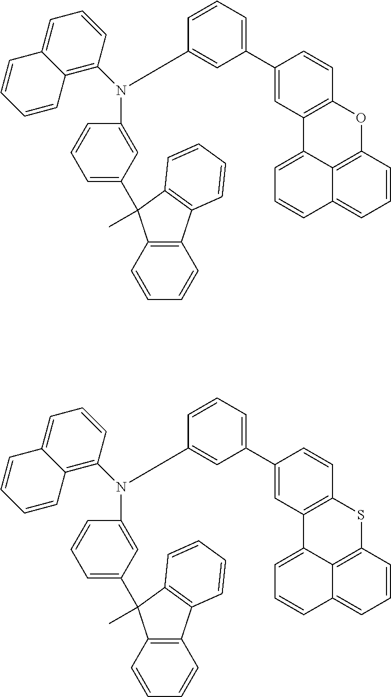 [SMILES: CC1(c2cccc(N(c3cccc(-c4ccc5c(c4)-c4cccc6cccc(c46)O5)c3)c3cccc4ccccc34)c2)c2ccccc2-c2ccccc21 ; CC1(c2cccc(N(c3cccc(-c4ccc5c(c4)-c4cccc6cccc(c46)S5)c3)c3cccc4ccccc34)c2)c2ccccc2-c2ccccc21]

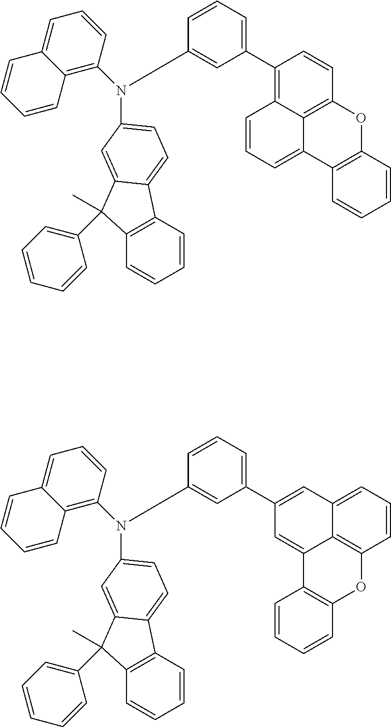 [SMILES: CC1(c2ccccc2)c2ccccc2-c2ccc(N(c3cccc(-c4ccc5c6c(cccc46)-c4ccccc4O5)c3)c3cccc4ccccc34)cc21 ; CC1(c2ccccc2)c2ccccc2-c2ccc(N(c3cccc(-c4cc5c6c(cccc6c4)Oc4ccccc4-5)c3)c3cccc4ccccc34)cc21]

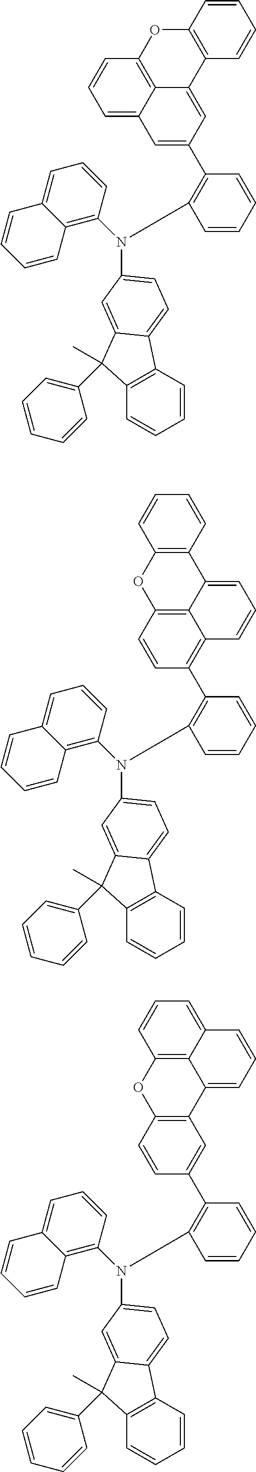

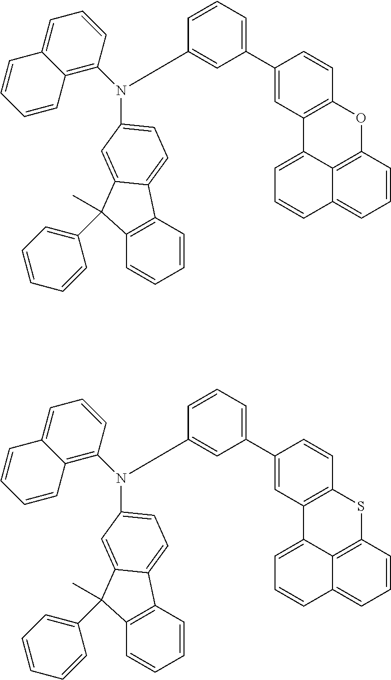 [SMILES: CC1(c2ccccc2)c2ccccc2-c2ccc(N(c3cccc(-c4ccc5c(c4)-c4cccc6cccc(c46)O5)c3)c3cccc4ccccc34)cc21 ; CC1(c2ccccc2)c2ccccc2-c2ccc(N(c3cccc(-c4ccc5c(c4)-c4cccc6cccc(c46)S5)c3)c3cccc4ccccc34)cc21]

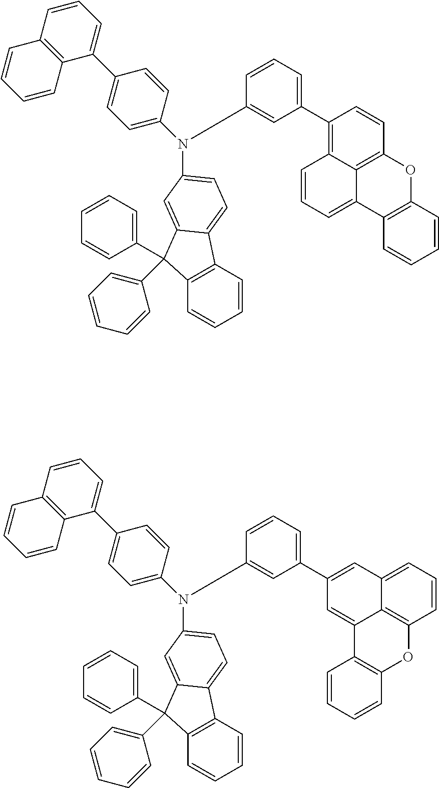 [SMILES: c1ccc(C2(c3ccccc3)c3ccccc3-c3ccc(N(c4ccc(-c5cccc6ccccc56)cc4)c4cccc(-c5ccc6c7c(cccc57)-c5ccccc5O6)c4)cc32)cc1 ; c1ccc(C2(c3ccccc3)c3ccccc3-c3ccc(N(c4ccc(-c5cccc6ccccc56)cc4)c4cccc(-c5cc6c7c(cccc7c5)Oc5ccccc5-6)c4)cc32)cc1]

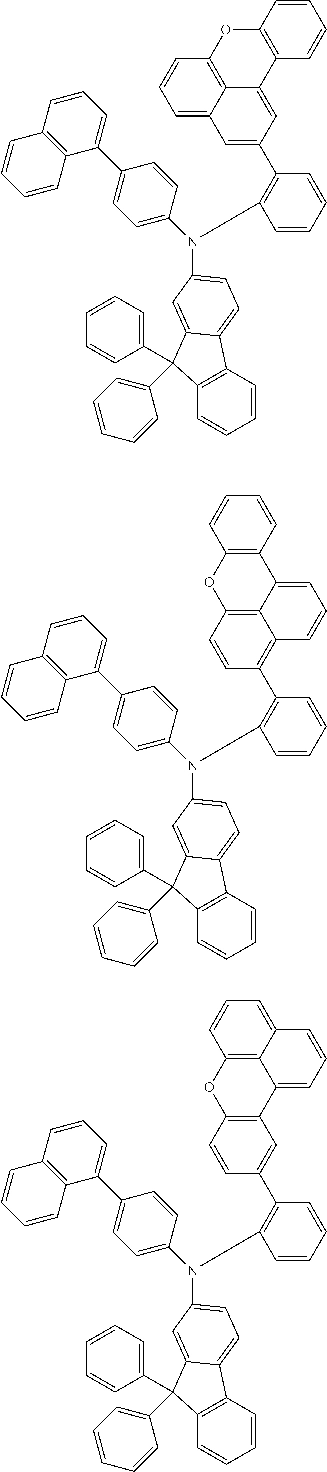

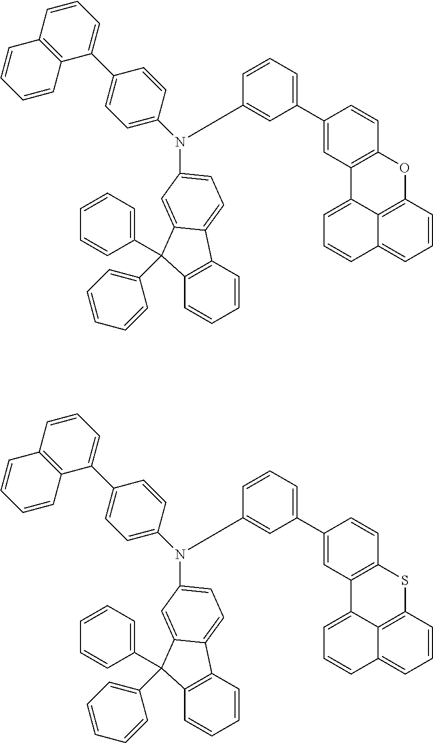 [SMILES: c1ccc(C2(c3ccccc3)c3ccccc3-c3ccc(N(c4ccc(-c5cccc6ccccc56)cc4)c4cccc(-c5ccc6c(c5)-c5cccc7cccc(c57)O6)c4)cc32)cc1 ; c1ccc(C2(c3ccccc3)c3ccccc3-c3ccc(N(c4ccc(-c5cccc6ccccc56)cc4)c4cccc(-c5ccc6c(c5)-c5cccc7cccc(c57)S6)c4)cc32)cc1]

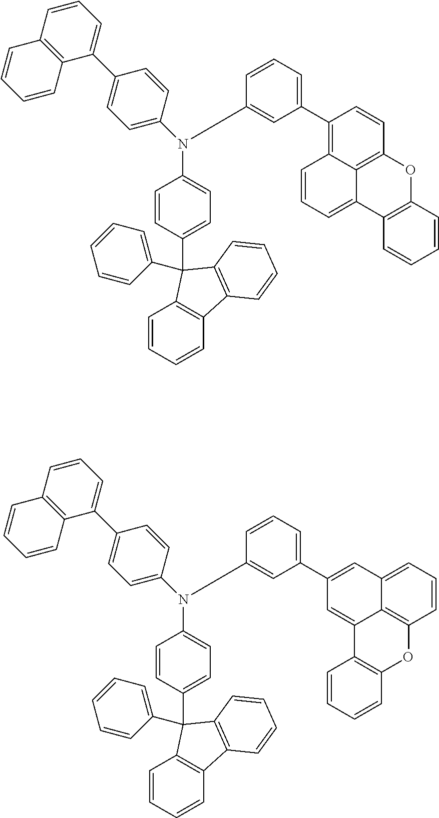 [SMILES: c1ccc(C2(c3ccc(N(c4ccc(-c5cccc6ccccc56)cc4)c4cccc(-c5ccc6c7c(cccc57)-c5ccccc5O6)c4)cc3)c3ccccc3-c3ccccc32)cc1 ; c1ccc(C2(c3ccc(N(c4ccc(-c5cccc6ccccc56)cc4)c4cccc(-c5cc6c7c(cccc7c5)Oc5ccccc5-6)c4)cc3)c3ccccc3-c3ccccc32)cc1]

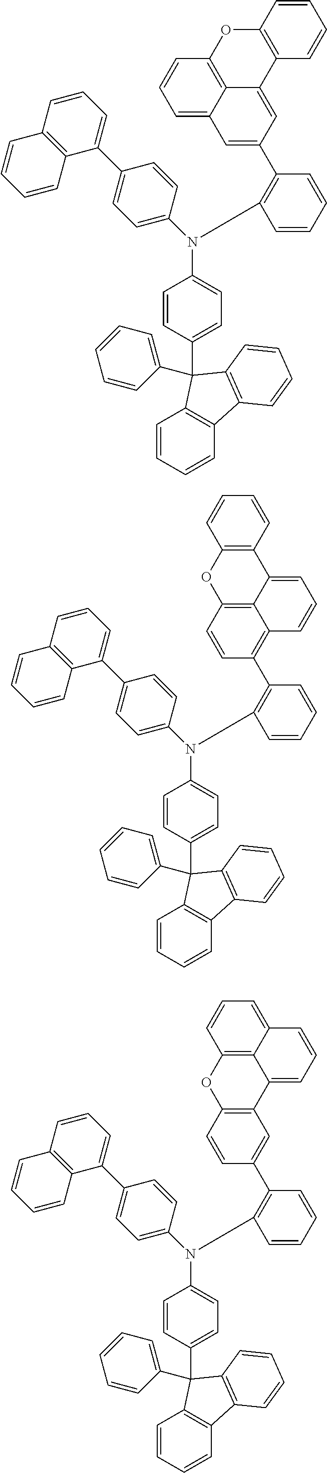

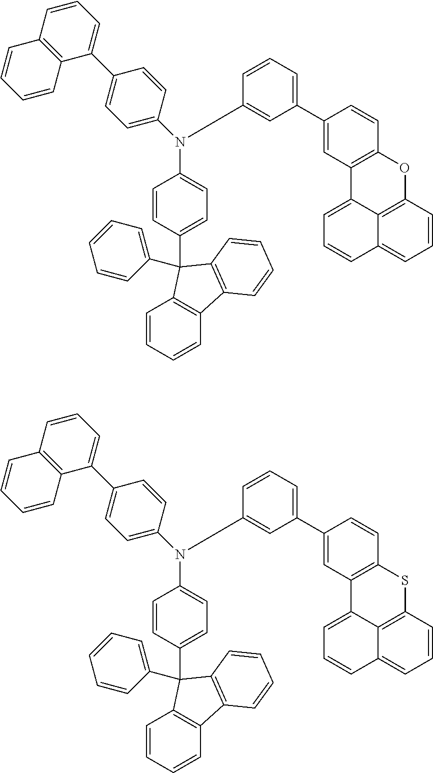 [SMILES: c1ccc(C2(c3ccc(N(c4ccc(-c5cccc6ccccc56)cc4)c4cccc(-c5ccc6c(c5)-c5cccc7cccc(c57)O6)c4)cc3)c3ccccc3-c3ccccc32)cc1 ; c1ccc(C2(c3ccc(N(c4ccc(-c5cccc6ccccc56)cc4)c4cccc(-c5ccc6c(c5)-c5cccc7cccc(c57)S6)c4)cc3)c3ccccc3-c3ccccc32)cc1]

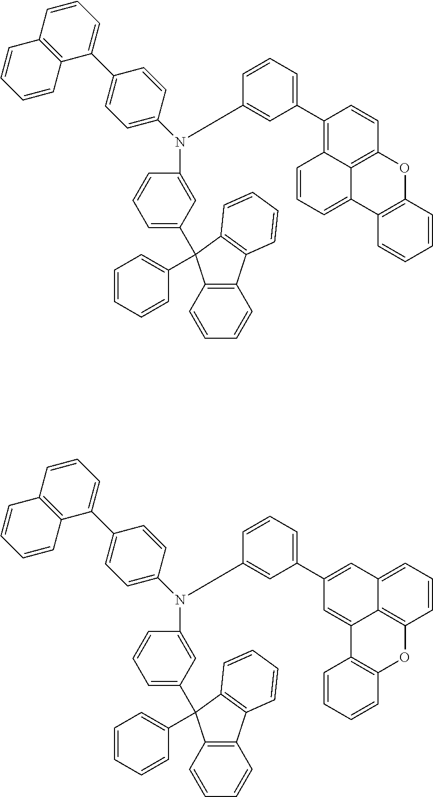 [SMILES: c1ccc(C2(c3cccc(N(c4ccc(-c5cccc6ccccc56)cc4)c4cccc(-c5ccc6c7c(cccc57)-c5ccccc5O6)c4)c3)c3ccccc3-c3ccccc32)cc1 ; c1ccc(C2(c3cccc(N(c4ccc(-c5cccc6ccccc56)cc4)c4cccc(-c5cc6c7c(cccc7c5)Oc5ccccc5-6)c4)c3)c3ccccc3-c3ccccc32)cc1]

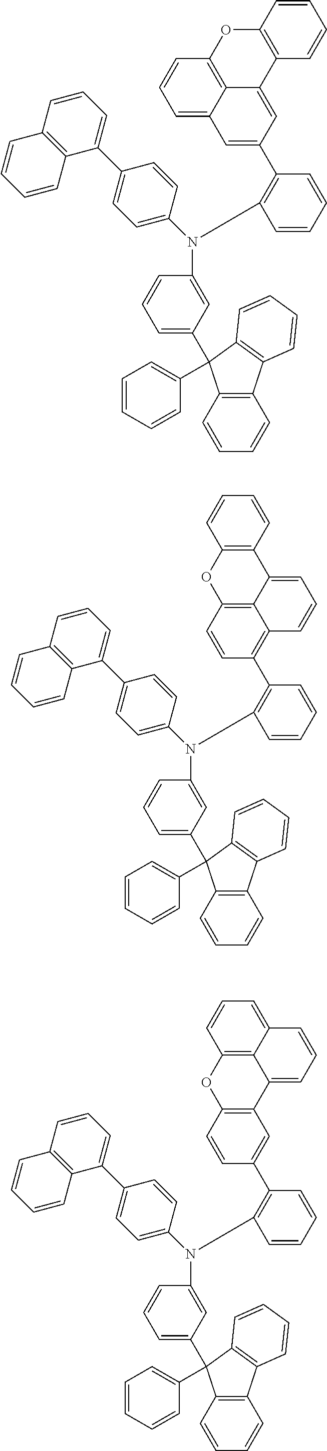 [SMILES: c1ccc(C2(c3cccc(N(c4ccc(-c5cccc6ccccc56)cc4)c4ccccc4-c4cc5c6c(cccc6c4)Oc4ccccc4-5)c3)c3ccccc3-c3ccccc32)cc1 ; c1ccc(C2(c3cccc(N(c4ccc(-c5cccc6ccccc56)cc4)c4ccccc4-c4ccc5c6c(cccc46)-c4ccccc4O5)c3)c3ccccc3-c3ccccc32)cc1 ; c1ccc(C2(c3cccc(N(c4ccc(-c5cccc6ccccc56)cc4)c4ccccc4-c4ccc5c(c4)-c4cccc6cccc(c46)O5)c3)c3ccccc3-c3ccccc32)cc1]

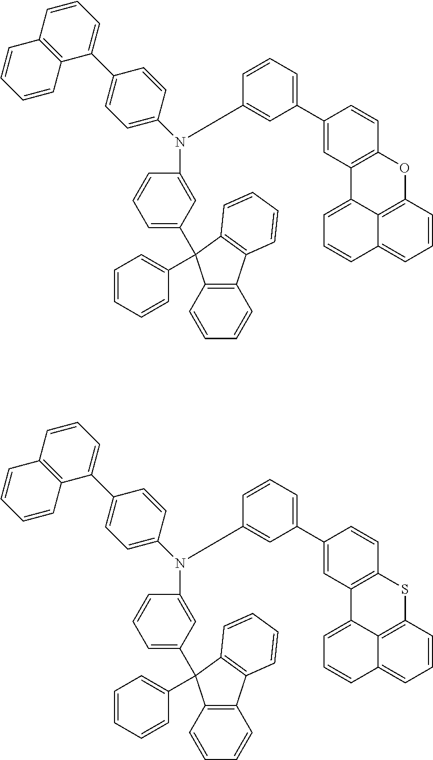 [SMILES: c1ccc(C2(c3cccc(N(c4ccc(-c5cccc6ccccc56)cc4)c4cccc(-c5ccc6c(c5)-c5cccc7cccc(c57)O6)c4)c3)c3ccccc3-c3ccccc32)cc1 ; c1ccc(C2(c3cccc(N(c4ccc(-c5cccc6ccccc56)cc4)c4cccc(-c5ccc6c(c5)-c5cccc7cccc(c57)S6)c4)c3)c3ccccc3-c3ccccc32)cc1]

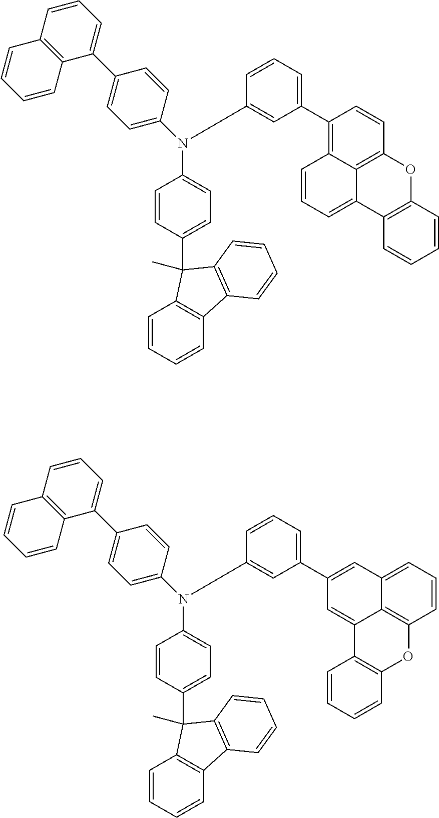 [SMILES: CC1(c2ccc(N(c3ccc(-c4cccc5ccccc45)cc3)c3cccc(-c4ccc5c6c(cccc46)-c4ccccc4O5)c3)cc2)c2ccccc2-c2ccccc21 ; CC1(c2ccc(N(c3ccc(-c4cccc5ccccc45)cc3)c3cccc(-c4cc5c6c(cccc6c4)Oc4ccccc4-5)c3)cc2)c2ccccc2-c2ccccc21]

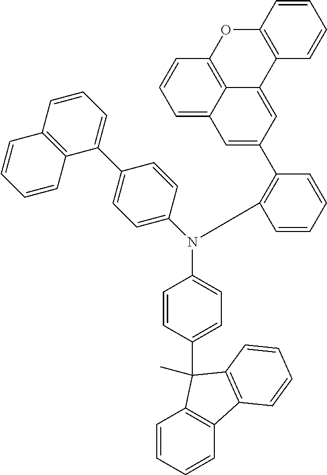 [SMILES: CC1(c2ccc(N(c3ccc(-c4cccc5ccccc45)cc3)c3ccccc3-c3cc4c5c(cccc5c3)Oc3ccccc3-4)cc2)c2ccccc2-c2ccccc21]

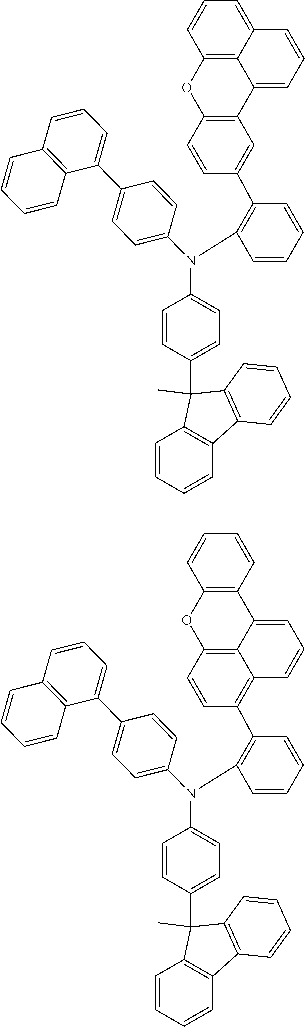

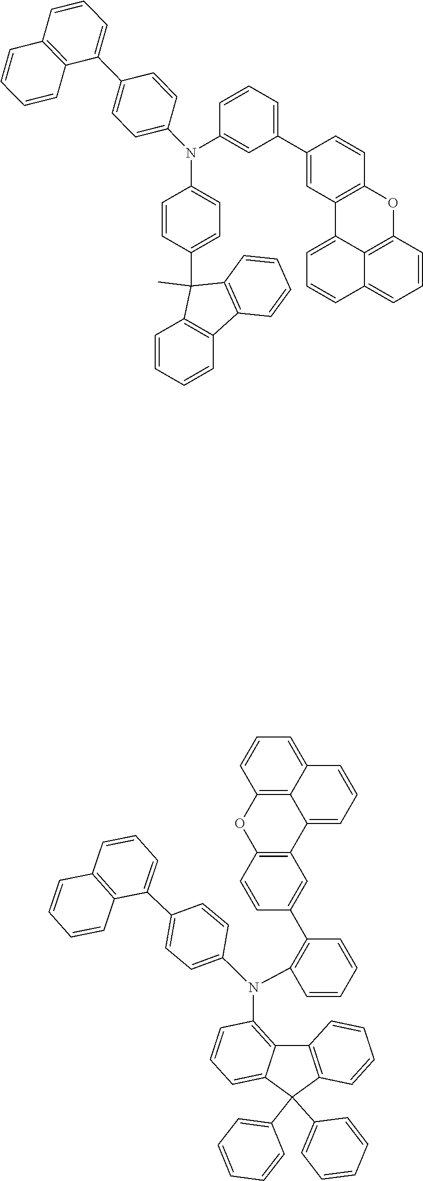 [SMILES: CC1(c2ccc(N(c3ccc(-c4cccc5ccccc45)cc3)c3cccc(-c4ccc5c(c4)-c4cccc6cccc(c46)O5)c3)cc2)c2ccccc2-c2ccccc21 ; c1ccc(C2(c3ccccc3)c3ccccc3-c3c(N(c4ccc(-c5cccc6ccccc56)cc4)c4ccccc4-c4ccc5c(c4)-c4cccc6cccc(c46)O5)cccc32)cc1]

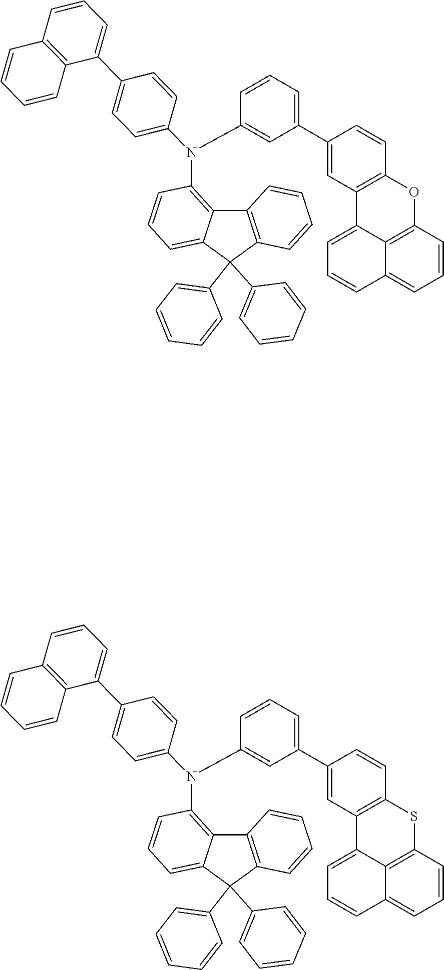 [SMILES: c1ccc(C2(c3ccccc3)c3ccccc3-c3c(N(c4ccc(-c5cccc6ccccc56)cc4)c4cccc(-c5ccc6c(c5)-c5cccc7cccc(c57)O6)c4)cccc32)cc1 ; c1ccc(C2(c3ccccc3)c3ccccc3-c3c(N(c4ccc(-c5cccc6ccccc56)cc4)c4cccc(-c5ccc6c(c5)-c5cccc7cccc(c57)S6)c4)cccc32)cc1]

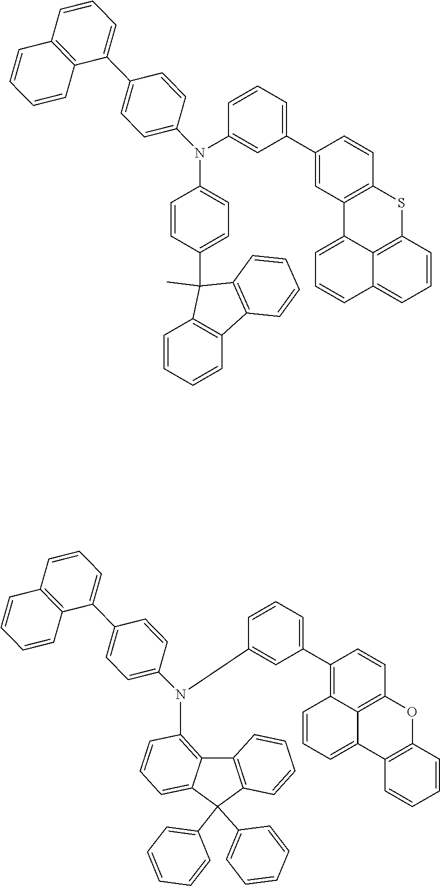 [SMILES: CC1(c2ccc(N(c3ccc(-c4cccc5ccccc45)cc3)c3cccc(-c4ccc5c(c4)-c4cccc6cccc(c46)S5)c3)cc2)c2ccccc2-c2ccccc21 ; c1ccc(C2(c3ccccc3)c3ccccc3-c3c(N(c4ccc(-c5cccc6ccccc56)cc4)c4cccc(-c5ccc6c7c(cccc57)-c5ccccc5O6)c4)cccc32)cc1]

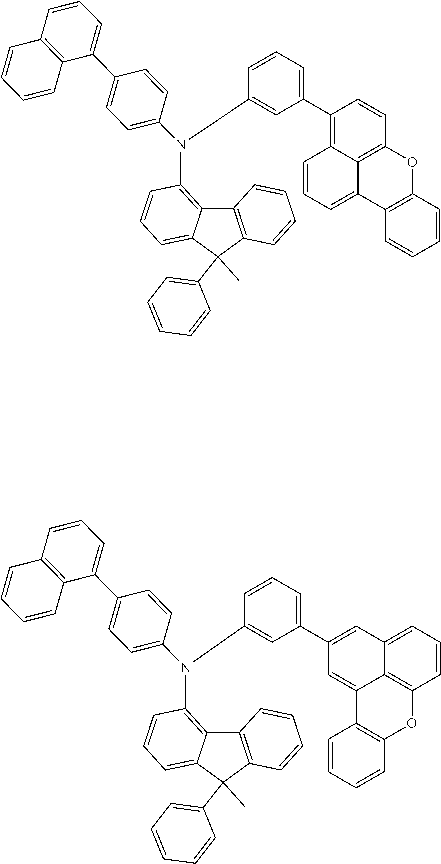 [SMILES: CC1(c2ccccc2)c2ccccc2-c2c(N(c3ccc(-c4cccc5ccccc45)cc3)c3cccc(-c4ccc5c6c(cccc46)-c4ccccc4O5)c3)cccc21 ; CC1(c2ccccc2)c2ccccc2-c2c(N(c3ccc(-c4cccc5ccccc45)cc3)c3cccc(-c4cc5c6c(cccc6c4)Oc4ccccc4-5)c3)cccc21]

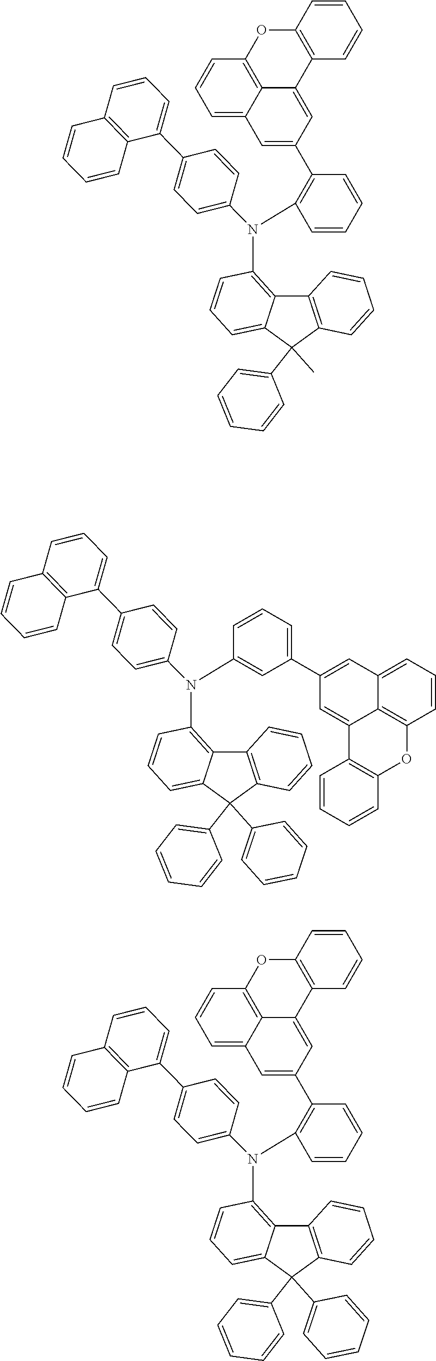 [SMILES: CC1(c2ccccc2)c2ccccc2-c2c(N(c3ccc(-c4cccc5ccccc45)cc3)c3ccccc3-c3cc4c5c(cccc5c3)Oc3ccccc3-4)cccc21 ; c1ccc(C2(c3ccccc3)c3ccccc3-c3c(N(c4ccc(-c5cccc6ccccc56)cc4)c4cccc(-c5cc6c7c(cccc7c5)Oc5ccccc5-6)c4)cccc32)cc1 ; c1ccc(C2(c3ccccc3)c3ccccc3-c3c(N(c4ccc(-c5cccc6ccccc56)cc4)c4ccccc4-c4cc5c6c(cccc6c4)Oc4ccccc4-5)cccc32)cc1]

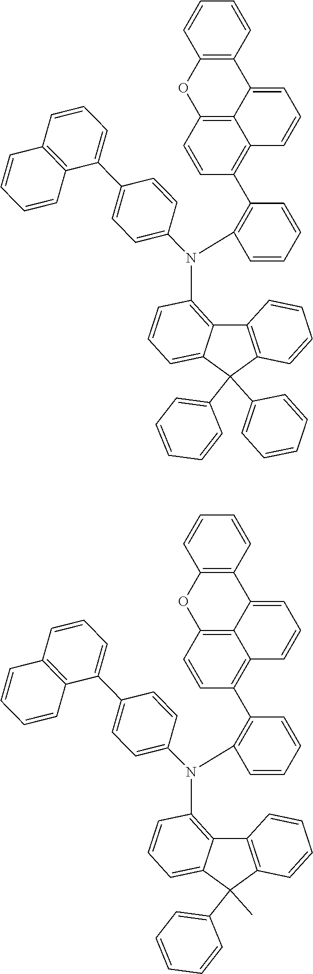 [SMILES: c1ccc(C2(c3ccccc3)c3ccccc3-c3c(N(c4ccc(-c5cccc6ccccc56)cc4)c4ccccc4-c4ccc5c6c(cccc46)-c4ccccc4O5)cccc32)cc1 ; CC1(c2ccccc2)c2ccccc2-c2c(N(c3ccc(-c4cccc5ccccc45)cc3)c3ccccc3-c3ccc4c5c(cccc35)-c3ccccc3O4)cccc21]

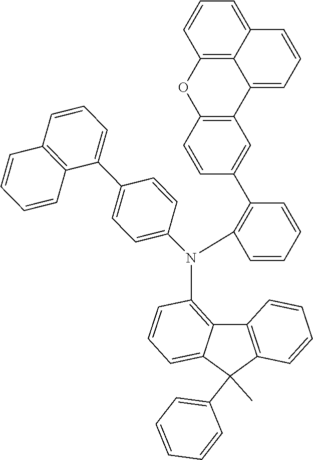 [SMILES: CC1(c2ccccc2)c2ccccc2-c2c(N(c3ccc(-c4cccc5ccccc45)cc3)c3ccccc3-c3ccc4c(c3)-c3cccc5cccc(c35)O4)cccc21]

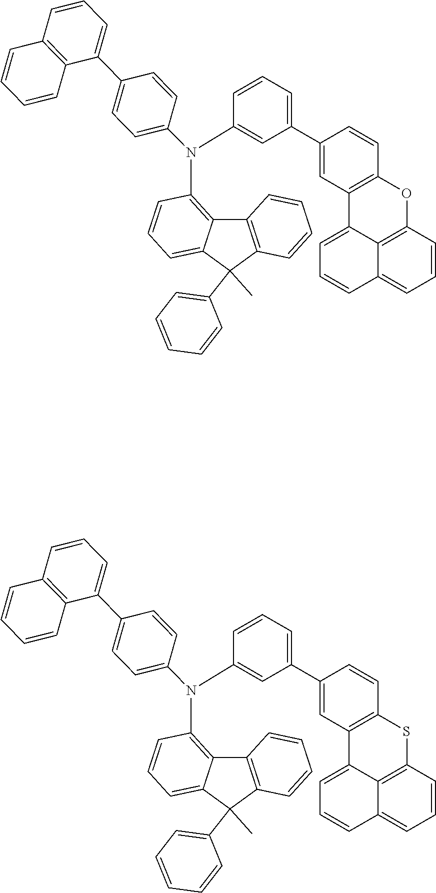 [SMILES: CC1(c2ccccc2)c2ccccc2-c2c(N(c3ccc(-c4cccc5ccccc45)cc3)c3cccc(-c4ccc5c(c4)-c4cccc6cccc(c46)O5)c3)cccc21 ; CC1(c2ccccc2)c2ccccc2-c2c(N(c3ccc(-c4cccc5ccccc45)cc3)c3cccc(-c4ccc5c(c4)-c4cccc6cccc(c46)S5)c3)cccc21]

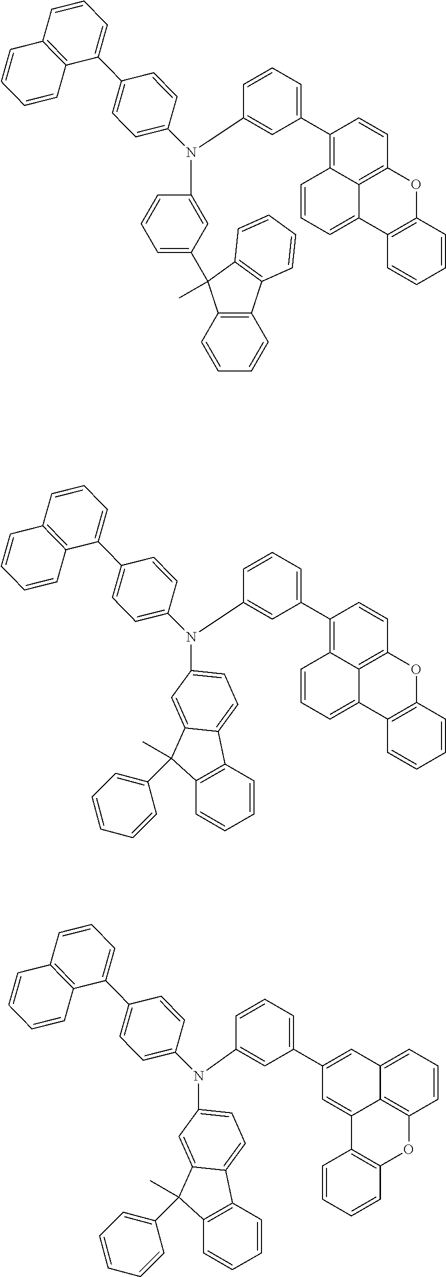 [SMILES: CC1(c2cccc(N(c3ccc(-c4cccc5ccccc45)cc3)c3cccc(-c4ccc5c6c(cccc46)-c4ccccc4O5)c3)c2)c2ccccc2-c2ccccc21 ; CC1(c2ccccc2)c2ccccc2-c2ccc(N(c3ccc(-c4cccc5ccccc45)cc3)c3cccc(-c4ccc5c6c(cccc46)-c4ccccc4O5)c3)cc21 ; CC1(c2ccccc2)c2ccccc2-c2ccc(N(c3ccc(-c4cccc5ccccc45)cc3)c3cccc(-c4cc5c6c(cccc6c4)Oc4ccccc4-5)c3)cc21]

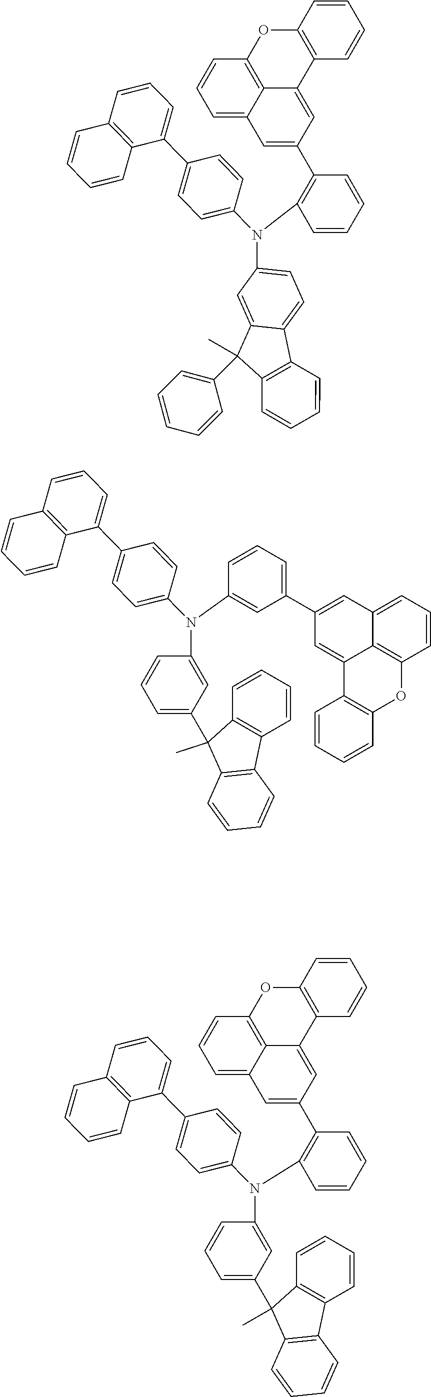

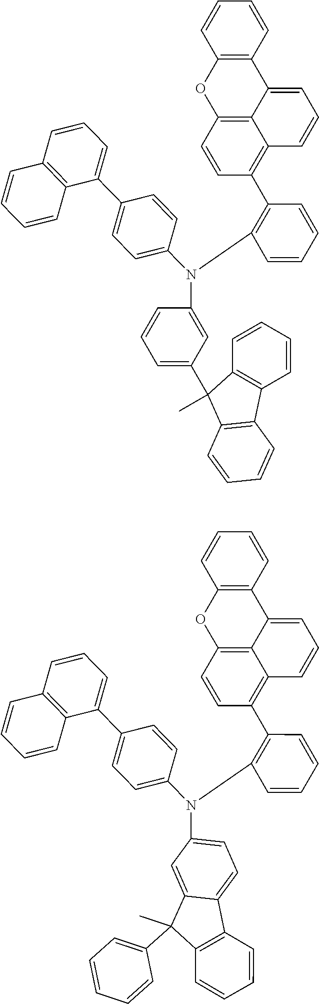 [SMILES: CC1(c2cccc(N(c3ccc(-c4cccc5ccccc45)cc3)c3ccccc3-c3ccc4c5c(cccc35)-c3ccccc3O4)c2)c2ccccc2-c2ccccc21 ; CC1(c2ccccc2)c2ccccc2-c2ccc(N(c3ccc(-c4cccc5ccccc45)cc3)c3ccccc3-c3ccc4c5c(cccc35)-c3ccccc3O4)cc21]

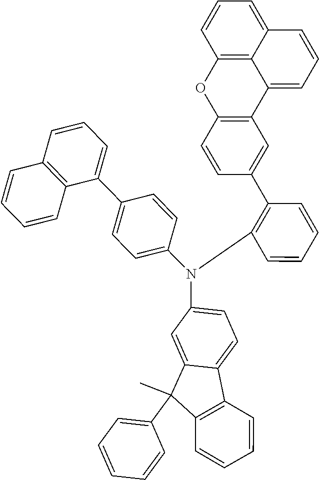 [SMILES: CC1(c2ccccc2)c2ccccc2-c2ccc(N(c3ccc(-c4cccc5ccccc45)cc3)c3ccccc3-c3ccc4c(c3)-c3cccc5cccc(c35)O4)cc21]

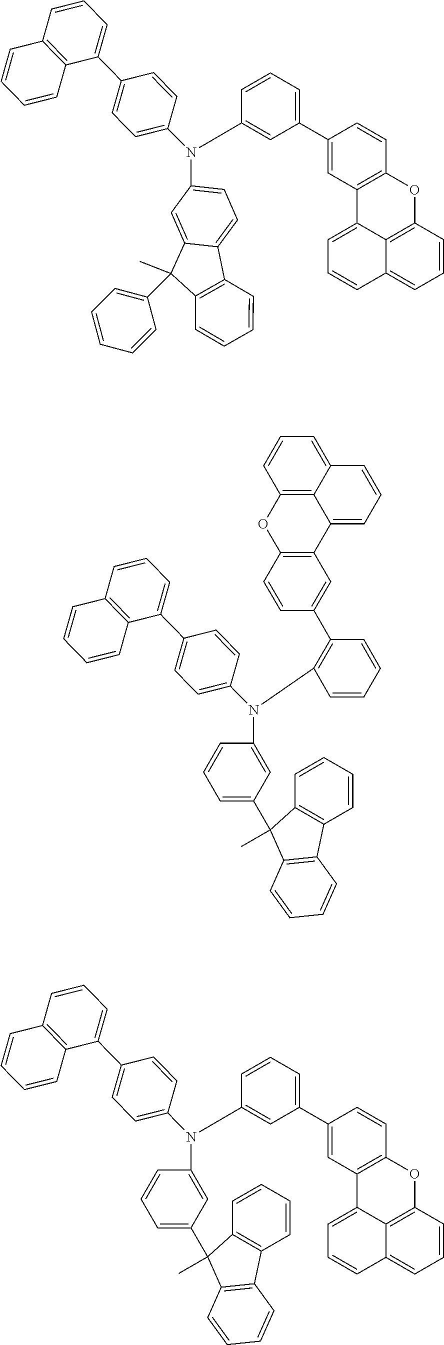 [SMILES: CC1(c2ccccc2)c2ccccc2-c2ccc(N(c3ccc(-c4cccc5ccccc45)cc3)c3cccc(-c4ccc5c(c4)-c4cccc6cccc(c46)O5)c3)cc21 ; CC1(c2cccc(N(c3ccc(-c4cccc5ccccc45)cc3)c3ccccc3-c3ccc4c(c3)-c3cccc5cccc(c35)O4)c2)c2ccccc2-c2ccccc21 ; CC1(c2cccc(N(c3ccc(-c4cccc5ccccc45)cc3)c3cccc(-c4ccc5c(c4)-c4cccc6cccc(c46)O5)c3)c2)c2ccccc2-c2ccccc21]

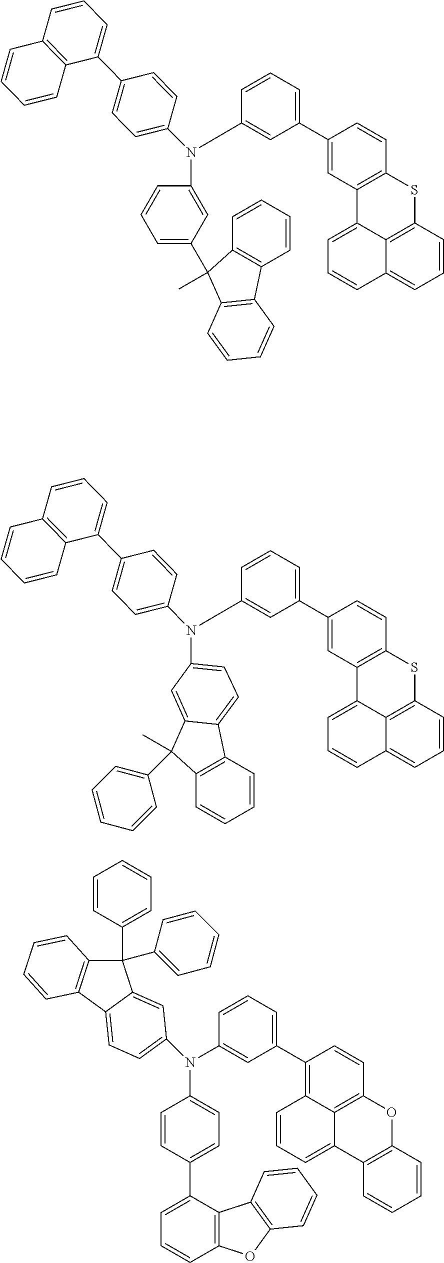

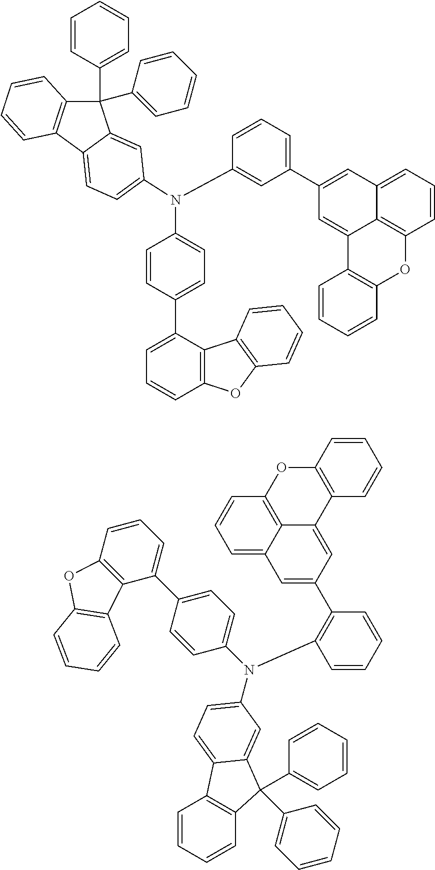 [SMILES: c1ccc(C2(c3ccccc3)c3ccccc3-c3ccc(N(c4ccc(-c5cccc6oc7ccccc7c56)cc4)c4cccc(-c5cc6c7c(cccc7c5)Oc5ccccc5-6)c4)cc32)cc1 ; c1ccc(C2(c3ccccc3)c3ccccc3-c3ccc(N(c4ccc(-c5cccc6oc7ccccc7c56)cc4)c4ccccc4-c4cc5c6c(cccc6c4)Oc4ccccc4-5)cc32)cc1]

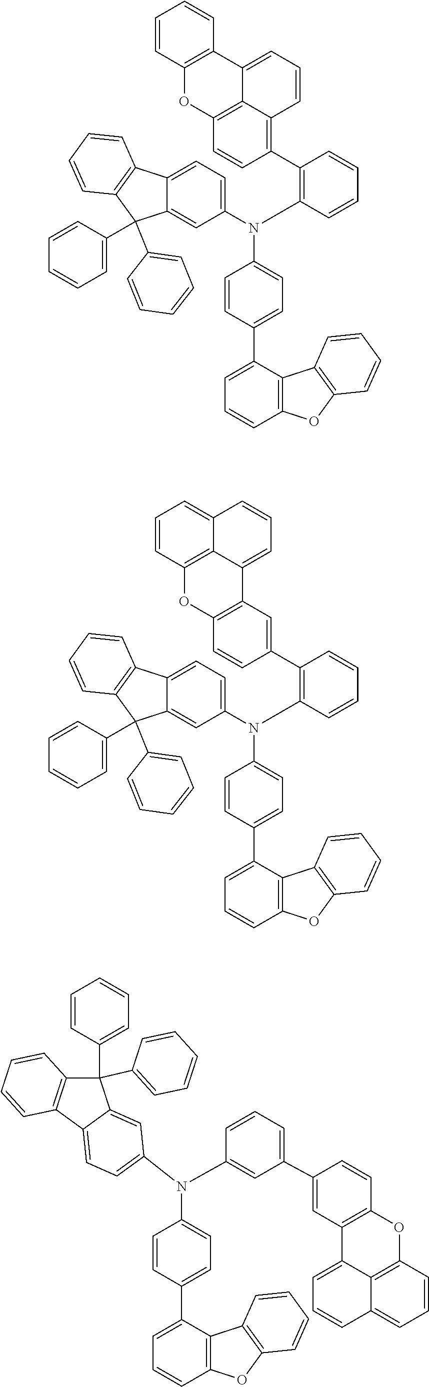 [SMILES: c1ccc(C2(c3ccccc3)c3ccccc3-c3ccc(N(c4ccc(-c5cccc6oc7ccccc7c56)cc4)c4ccccc4-c4ccc5c6c(cccc46)-c4ccccc4O5)cc32)cc1 ; c1ccc(C2(c3ccccc3)c3ccccc3-c3ccc(N(c4ccc(-c5cccc6oc7ccccc7c56)cc4)c4ccccc4-c4ccc5c(c4)-c4cccc6cccc(c46)O5)cc32)cc1 ; c1ccc(C2(c3ccccc3)c3ccccc3-c3ccc(N(c4ccc(-c5cccc6oc7ccccc7c56)cc4)c4cccc(-c5ccc6c(c5)-c5cccc7cccc(c57)O6)c4)cc32)cc1]

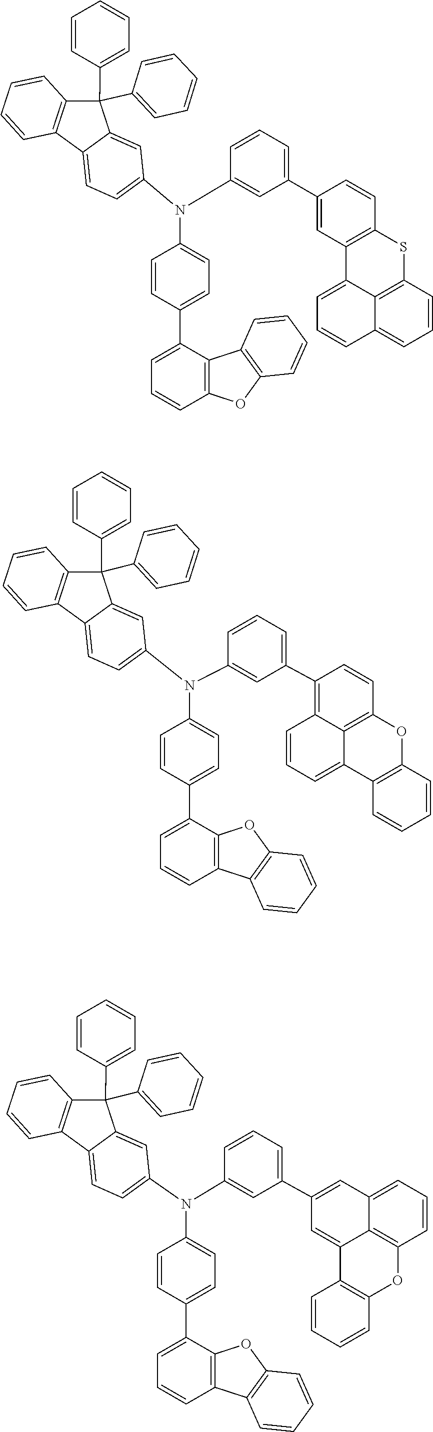 [SMILES: c1ccc(C2(c3ccccc3)c3ccccc3-c3ccc(N(c4ccc(-c5cccc6oc7ccccc7c56)cc4)c4cccc(-c5ccc6c(c5)-c5cccc7cccc(c57)S6)c4)cc32)cc1 ; c1ccc(C2(c3ccccc3)c3ccccc3-c3ccc(N(c4ccc(-c5cccc6c5oc5ccccc56)cc4)c4cccc(-c5ccc6c7c(cccc57)-c5ccccc5O6)c4)cc32)cc1 ; c1ccc(C2(c3ccccc3)c3ccccc3-c3ccc(N(c4ccc(-c5cccc6c5oc5ccccc56)cc4)c4cccc(-c5cc6c7c(cccc7c5)Oc5ccccc5-6)c4)cc32)cc1]

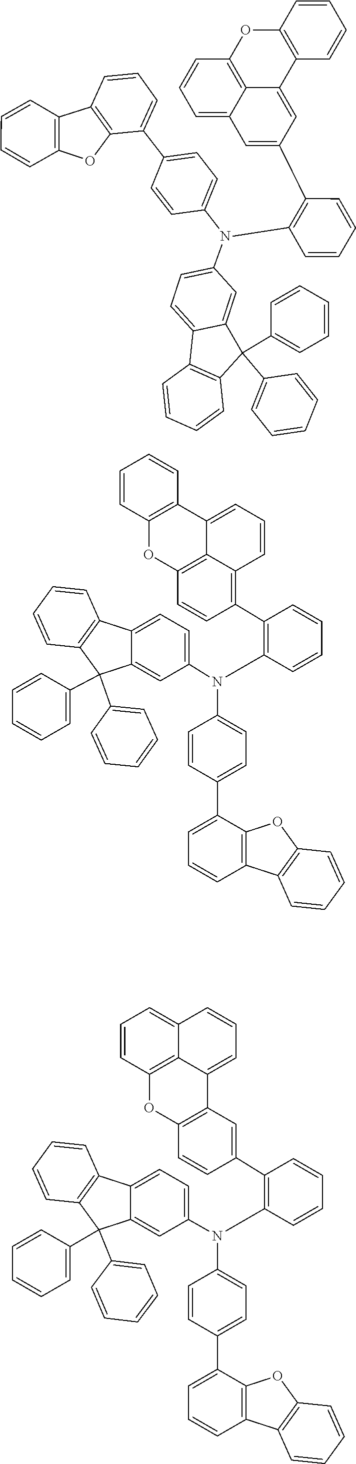

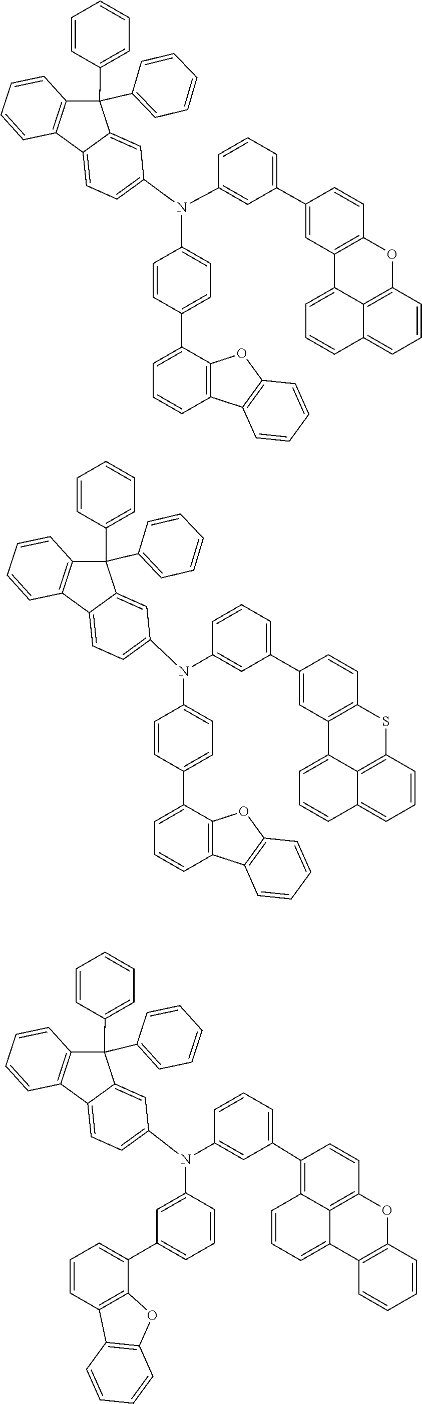 [SMILES: c1ccc(C2(c3ccccc3)c3ccccc3-c3ccc(N(c4ccc(-c5cccc6c5oc5ccccc56)cc4)c4cccc(-c5ccc6c(c5)-c5cccc7cccc(c57)O6)c4)cc32)cc1 ; c1ccc(C2(c3ccccc3)c3ccccc3-c3ccc(N(c4ccc(-c5cccc6c5oc5ccccc56)cc4)c4cccc(-c5ccc6c(c5)-c5cccc7cccc(c57)S6)c4)cc32)cc1 ; c1ccc(C2(c3ccccc3)c3ccccc3-c3ccc(N(c4cccc(-c5ccc6c7c(cccc57)-c5ccccc5O6)c4)c4cccc(-c5cccc6c5oc5ccccc56)c4)cc32)cc1]

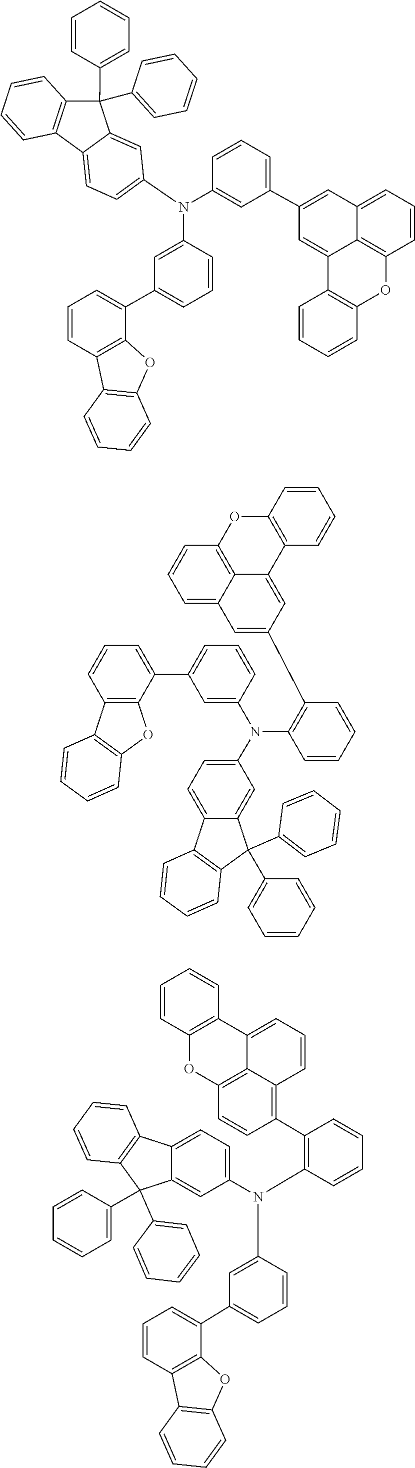 [SMILES: c1ccc(C2(c3ccccc3)c3ccccc3-c3ccc(N(c4cccc(-c5cc6c7c(cccc7c5)Oc5ccccc5-6)c4)c4cccc(-c5cccc6c5oc5ccccc56)c4)cc32)cc1 ; c1ccc(C2(c3ccccc3)c3ccccc3-c3ccc(N(c4cccc(-c5cccc6c5oc5ccccc56)c4)c4ccccc4-c4cc5c6c(cccc6c4)Oc4ccccc4-5)cc32)cc1 ; c1ccc(C2(c3ccccc3)c3ccccc3-c3ccc(N(c4cccc(-c5cccc6c5oc5ccccc56)c4)c4ccccc4-c4ccc5c6c(cccc46)-c4ccccc4O5)cc32)cc1]

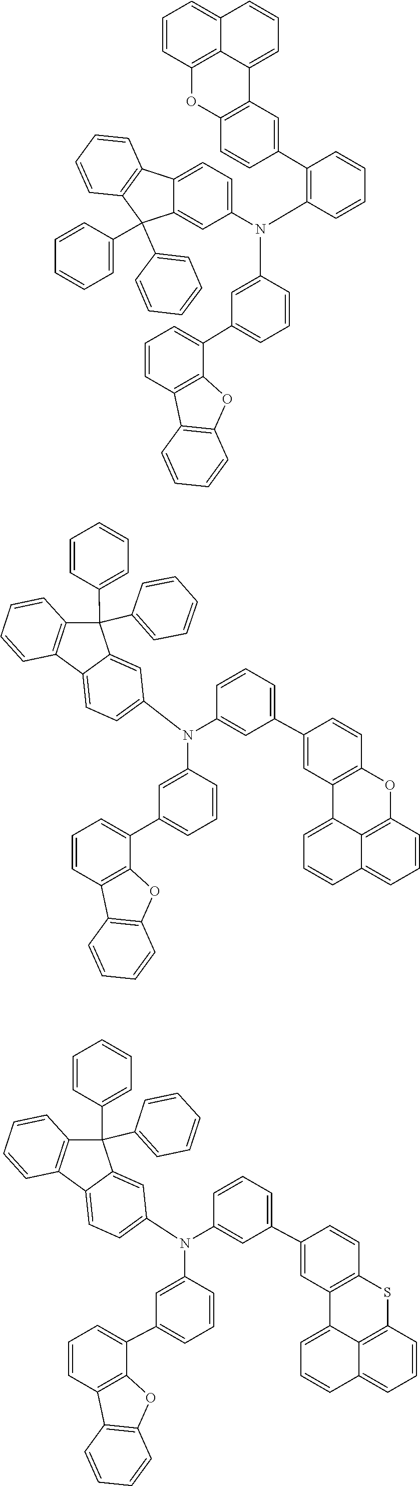 [SMILES: c1ccc(C2(c3ccccc3)c3ccccc3-c3ccc(N(c4cccc(-c5cccc6c5oc5ccccc56)c4)c4ccccc4-c4ccc5c(c4)-c4cccc6cccc(c46)O5)cc32)cc1 ; c1ccc(C2(c3ccccc3)c3ccccc3-c3ccc(N(c4cccc(-c5ccc6c(c5)-c5cccc7cccc(c57)O6)c4)c4cccc(-c5cccc6c5oc5ccccc56)c4)cc32)cc1 ; c1ccc(C2(c3ccccc3)c3ccccc3-c3ccc(N(c4cccc(-c5ccc6c(c5)-c5cccc7cccc(c57)S6)c4)c4cccc(-c5cccc6c5oc5ccccc56)c4)cc32)cc1]

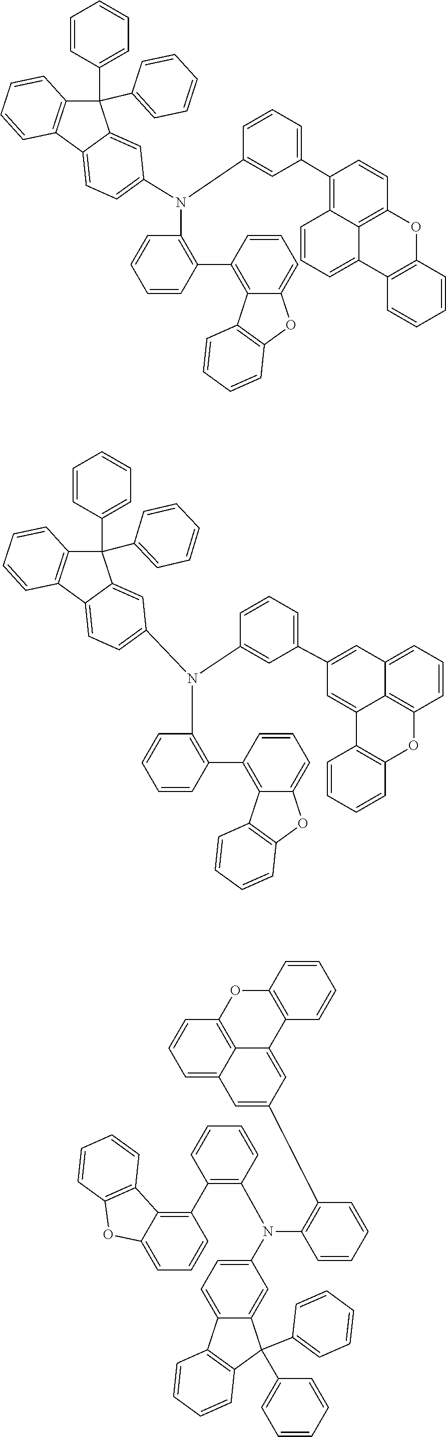 [SMILES: c1ccc(C2(c3ccccc3)c3ccccc3-c3ccc(N(c4cccc(-c5ccc6c7c(cccc57)-c5ccccc5O6)c4)c4ccccc4-c4cccc5oc6ccccc6c45)cc32)cc1 ; c1ccc(C2(c3ccccc3)c3ccccc3-c3ccc(N(c4cccc(-c5cc6c7c(cccc7c5)Oc5ccccc5-6)c4)c4ccccc4-c4cccc5oc6ccccc6c45)cc32)cc1 ; c1ccc(C2(c3ccccc3)c3ccccc3-c3ccc(N(c4ccccc4-c4cc5c6c(cccc6c4)Oc4ccccc4-5)c4ccccc4-c4cccc5oc6ccccc6c45)cc32)cc1]

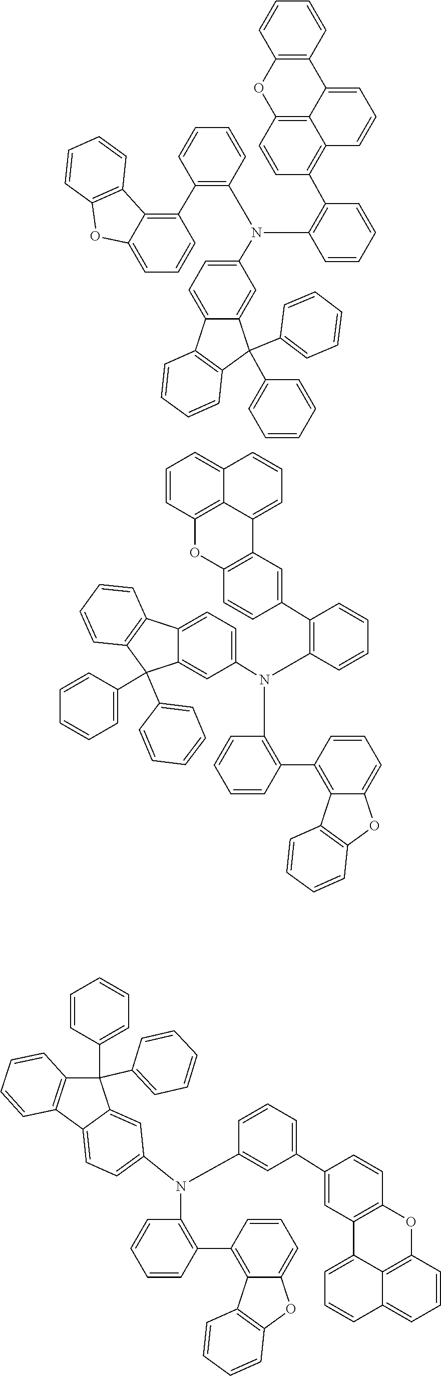

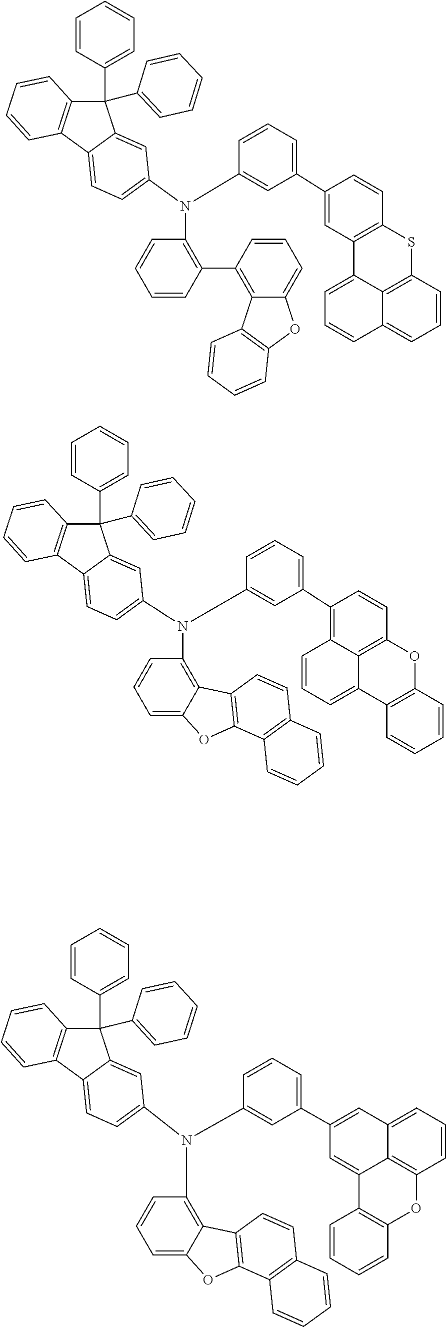 [SMILES: c1ccc(C2(c3ccccc3)c3ccccc3-c3ccc(N(c4cccc(-c5ccc6c(c5)-c5cccc7cccc(c57)S6)c4)c4ccccc4-c4cccc5oc6ccccc6c45)cc32)cc1 ; c1ccc(C2(c3ccccc3)c3ccccc3-c3ccc(N(c4cccc(-c5ccc6c7c(cccc57)-c5ccccc5O6)c4)c4cccc5oc6c7ccccc7ccc6c45)cc32)cc1 ; c1ccc(C2(c3ccccc3)c3ccccc3-c3ccc(N(c4cccc(-c5cc6c7c(cccc7c5)Oc5ccccc5-6)c4)c4cccc5oc6c7ccccc7ccc6c45)cc32)cc1]

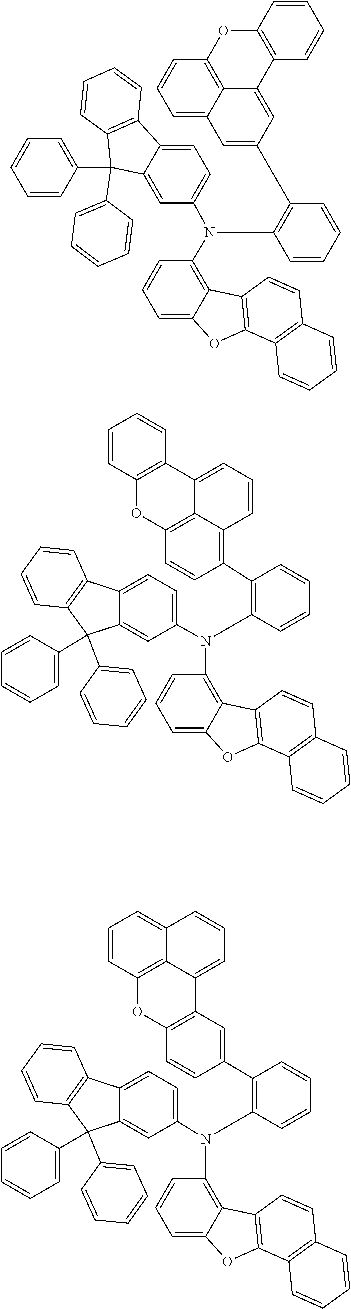 [SMILES: c1ccc(C2(c3ccccc3)c3ccccc3-c3ccc(N(c4ccccc4-c4cc5c6c(cccc6c4)Oc4ccccc4-5)c4cccc5oc6c7ccccc7ccc6c45)cc32)cc1 ; c1ccc(C2(c3ccccc3)c3ccccc3-c3ccc(N(c4ccccc4-c4ccc5c6c(cccc46)-c4ccccc4O5)c4cccc5oc6c7ccccc7ccc6c45)cc32)cc1 ; c1ccc(C2(c3ccccc3)c3ccccc3-c3ccc(N(c4ccccc4-c4ccc5c(c4)-c4cccc6cccc(c46)O5)c4cccc5oc6c7ccccc7ccc6c45)cc32)cc1]

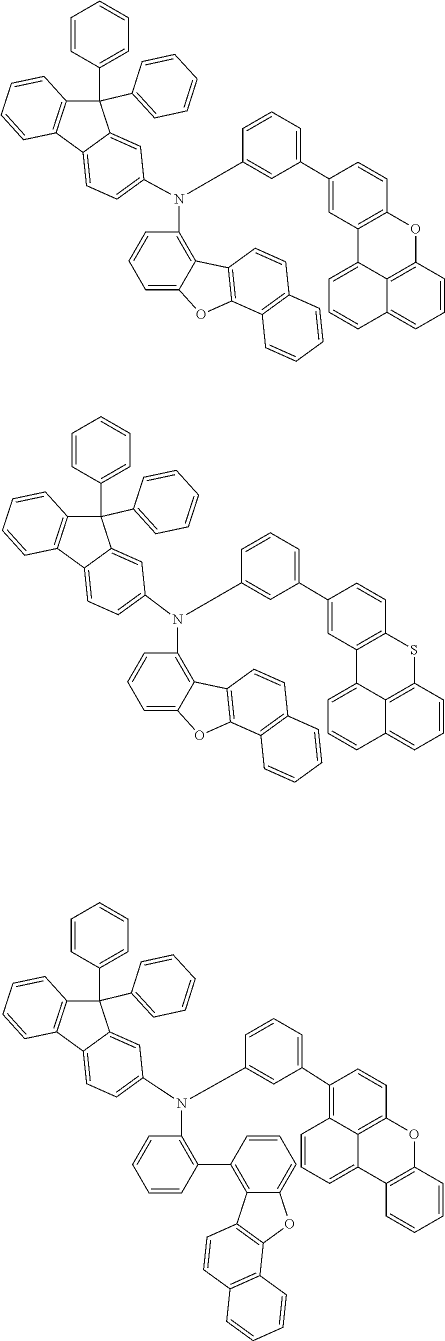 [SMILES: c1ccc(C2(c3ccccc3)c3ccccc3-c3ccc(N(c4cccc(-c5ccc6c(c5)-c5cccc7cccc(c57)O6)c4)c4cccc5oc6c7ccccc7ccc6c45)cc32)cc1 ; c1ccc(C2(c3ccccc3)c3ccccc3-c3ccc(N(c4cccc(-c5ccc6c(c5)-c5cccc7cccc(c57)S6)c4)c4cccc5oc6c7ccccc7ccc6c45)cc32)cc1 ; c1ccc(C2(c3ccccc3)c3ccccc3-c3ccc(N(c4cccc(-c5ccc6c7c(cccc57)-c5ccccc5O6)c4)c4ccccc4-c4cccc5oc6c7ccccc7ccc6c45)cc32)cc1]

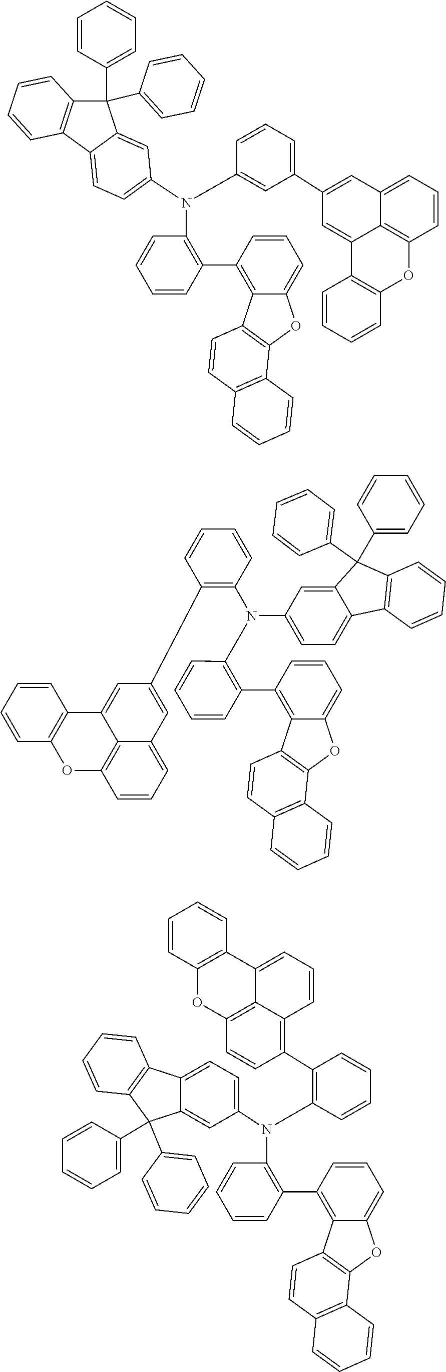 [SMILES: c1ccc(C2(c3ccccc3)c3ccccc3-c3ccc(N(c4cccc(-c5cc6c7c(cccc7c5)Oc5ccccc5-6)c4)c4ccccc4-c4cccc5oc6c7ccccc7ccc6c45)cc32)cc1 ; c1ccc(C2(c3ccccc3)c3ccccc3-c3ccc(N(c4ccccc4-c4cc5c6c(cccc6c4)Oc4ccccc4-5)c4ccccc4-c4cccc5oc6c7ccccc7ccc6c45)cc32)cc1 ; c1ccc(C2(c3ccccc3)c3ccccc3-c3ccc(N(c4ccccc4-c4ccc5c6c(cccc46)-c4ccccc4O5)c4ccccc4-c4cccc5oc6c7ccccc7ccc6c45)cc32)cc1]

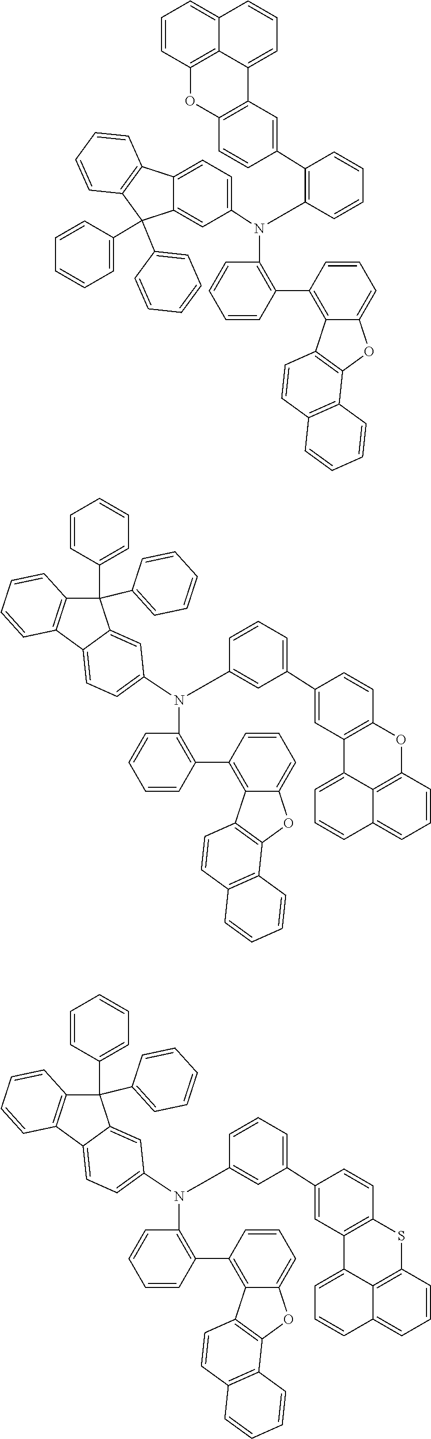 [SMILES: c1ccc(C2(c3ccccc3)c3ccccc3-c3ccc(N(c4ccccc4-c4ccc5c(c4)-c4cccc6cccc(c46)O5)c4ccccc4-c4cccc5oc6c7ccccc7ccc6c45)cc32)cc1 ; c1ccc(C2(c3ccccc3)c3ccccc3-c3ccc(N(c4cccc(-c5ccc6c(c5)-c5cccc7cccc(c57)O6)c4)c4ccccc4-c4cccc5oc6c7ccccc7ccc6c45)cc32)cc1 ; c1ccc(C2(c3ccccc3)c3ccccc3-c3ccc(N(c4cccc(-c5ccc6c(c5)-c5cccc7cccc(c57)S6)c4)c4ccccc4-c4cccc5oc6c7ccccc7ccc6c45)cc32)cc1]

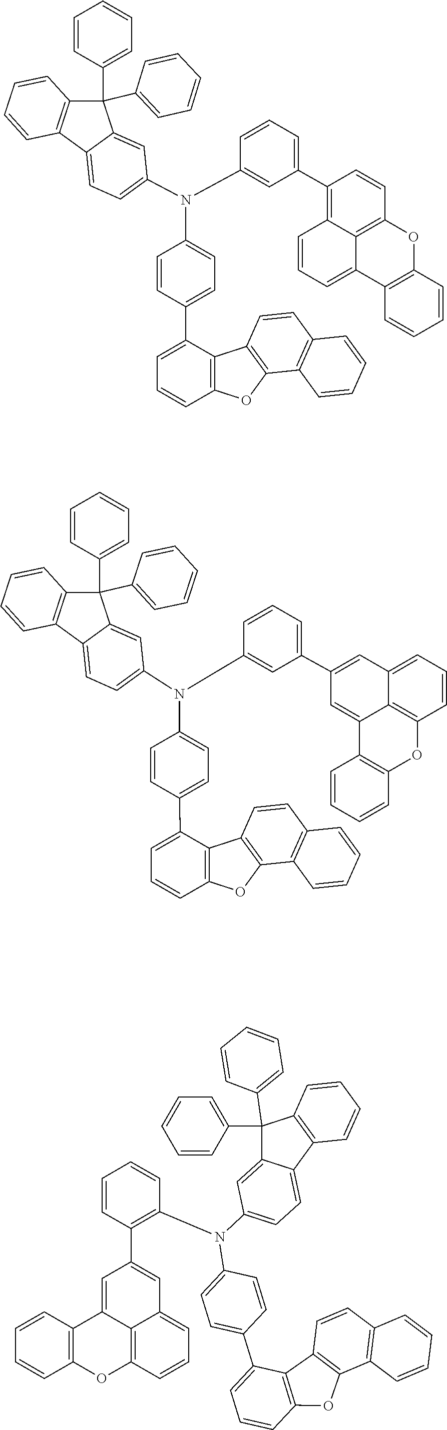 [SMILES: c1ccc(C2(c3ccccc3)c3ccccc3-c3ccc(N(c4ccc(-c5cccc6oc7c8ccccc8ccc7c56)cc4)c4cccc(-c5ccc6c7c(cccc57)-c5ccccc5O6)c4)cc32)cc1 ; c1ccc(C2(c3ccccc3)c3ccccc3-c3ccc(N(c4ccc(-c5cccc6oc7c8ccccc8ccc7c56)cc4)c4cccc(-c5cc6c7c(cccc7c5)Oc5ccccc5-6)c4)cc32)cc1 ; c1ccc(C2(c3ccccc3)c3ccccc3-c3ccc(N(c4ccc(-c5cccc6oc7c8ccccc8ccc7c56)cc4)c4ccccc4-c4cc5c6c(cccc6c4)Oc4ccccc4-5)cc32)cc1]

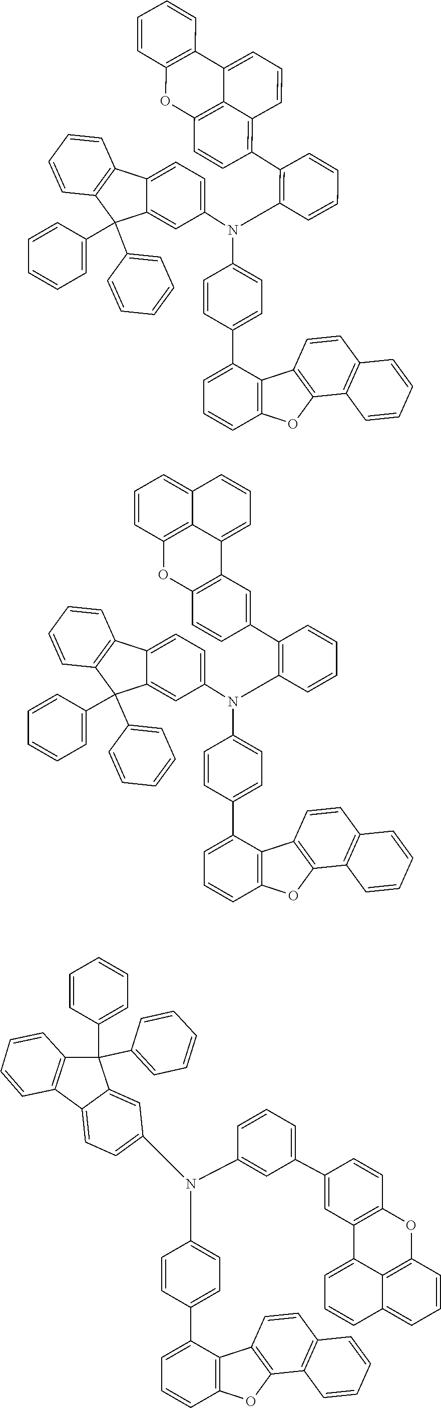

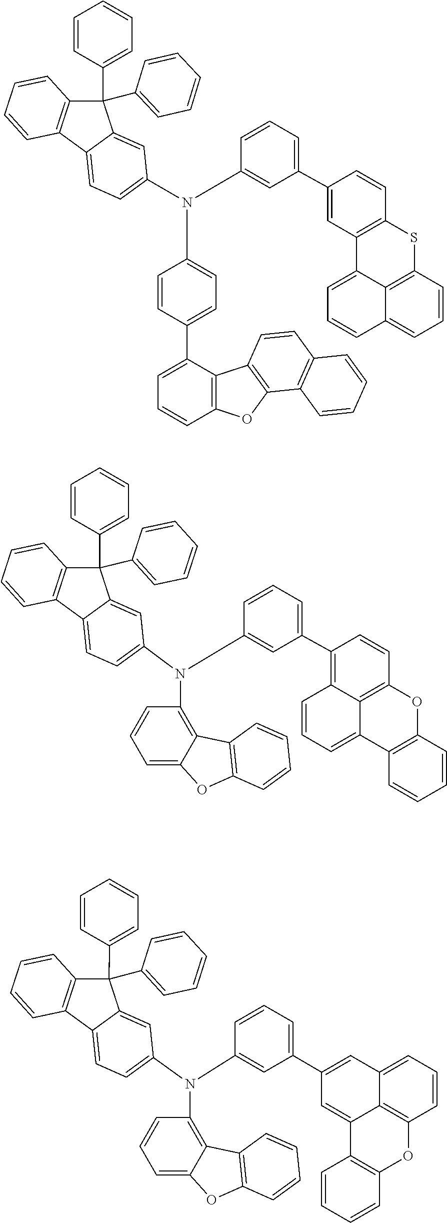 [SMILES: c1ccc(C2(c3ccccc3)c3ccccc3-c3ccc(N(c4ccc(-c5cccc6oc7c8ccccc8ccc7c56)cc4)c4cccc(-c5ccc6c(c5)-c5cccc7cccc(c57)S6)c4)cc32)cc1 ; c1ccc(C2(c3ccccc3)c3ccccc3-c3ccc(N(c4cccc(-c5ccc6c7c(cccc57)-c5ccccc5O6)c4)c4cccc5oc6ccccc6c45)cc32)cc1 ; c1ccc(C2(c3ccccc3)c3ccccc3-c3ccc(N(c4cccc(-c5cc6c7c(cccc7c5)Oc5ccccc5-6)c4)c4cccc5oc6ccccc6c45)cc32)cc1]

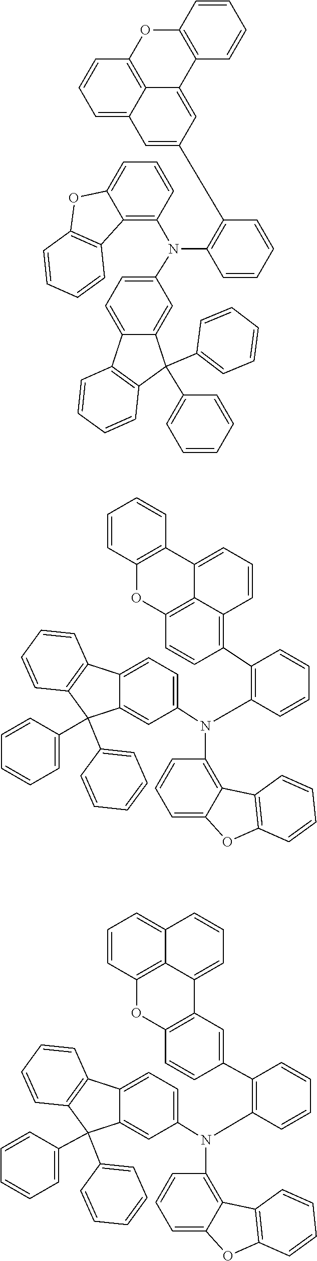 [SMILES: c1ccc(C2(c3ccccc3)c3ccccc3-c3ccc(N(c4ccccc4-c4cc5c6c(cccc6c4)Oc4ccccc4-5)c4cccc5oc6ccccc6c45)cc32)cc1 ; c1ccc(C2(c3ccccc3)c3ccccc3-c3ccc(N(c4ccccc4-c4ccc5c6c(cccc46)-c4ccccc4O5)c4cccc5oc6ccccc6c45)cc32)cc1 ; c1ccc(C2(c3ccccc3)c3ccccc3-c3ccc(N(c4ccccc4-c4ccc5c(c4)-c4cccc6cccc(c46)O5)c4cccc5oc6ccccc6c45)cc32)cc1]

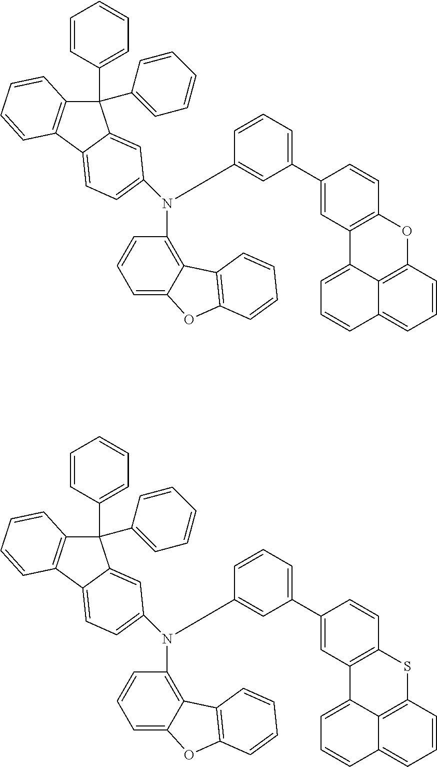 [SMILES: c1ccc(C2(c3ccccc3)c3ccccc3-c3ccc(N(c4cccc(-c5ccc6c(c5)-c5cccc7cccc(c57)O6)c4)c4cccc5oc6ccccc6c45)cc32)cc1 ; c1ccc(C2(c3ccccc3)c3ccccc3-c3ccc(N(c4cccc(-c5ccc6c(c5)-c5cccc7cccc(c57)S6)c4)c4cccc5oc6ccccc6c45)cc32)cc1]

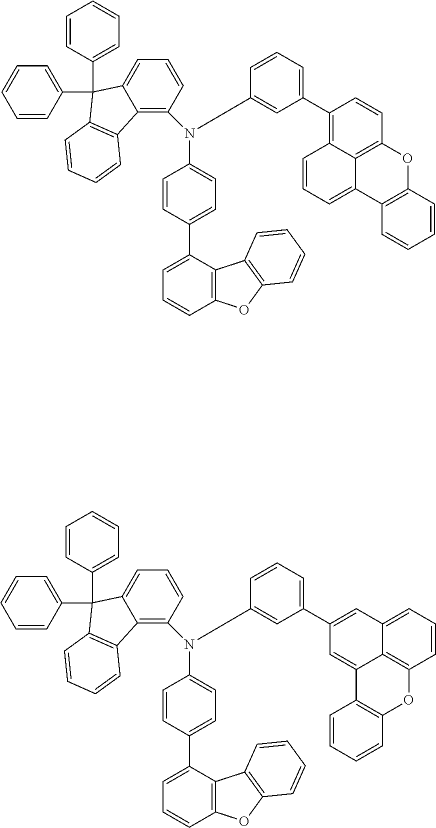 [SMILES: c1ccc(C2(c3ccccc3)c3ccccc3-c3c(N(c4ccc(-c5cccc6oc7ccccc7c56)cc4)c4cccc(-c5ccc6c7c(cccc57)-c5ccccc5O6)c4)cccc32)cc1 ; c1ccc(C2(c3ccccc3)c3ccccc3-c3c(N(c4ccc(-c5cccc6oc7ccccc7c56)cc4)c4cccc(-c5cc6c7c(cccc7c5)Oc5ccccc5-6)c4)cccc32)cc1]

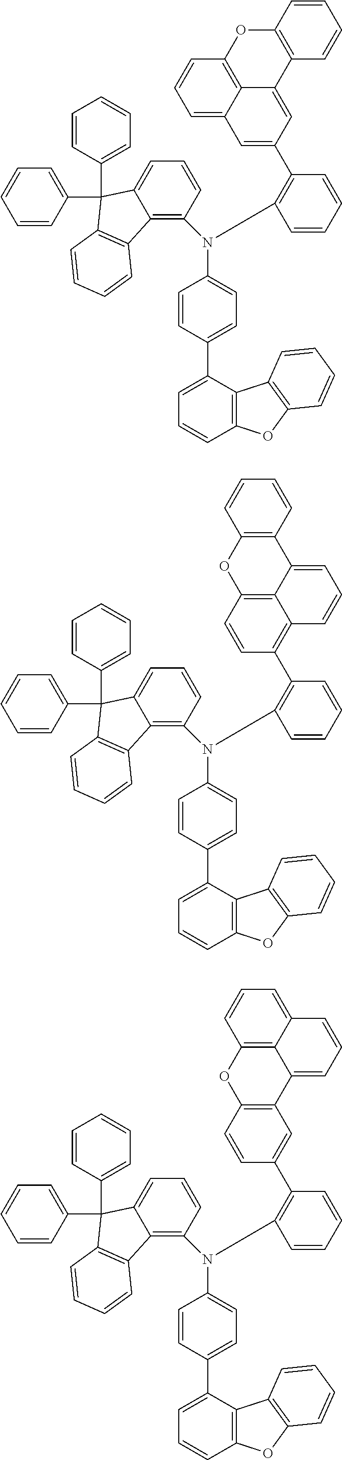

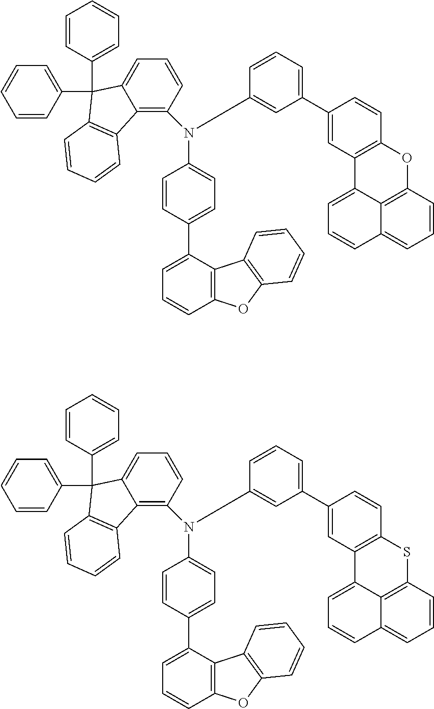 [SMILES: c1ccc(C2(c3ccccc3)c3ccccc3-c3c(N(c4ccc(-c5cccc6oc7ccccc7c56)cc4)c4cccc(-c5ccc6c(c5)-c5cccc7cccc(c57)O6)c4)cccc32)cc1 ; c1ccc(C2(c3ccccc3)c3ccccc3-c3c(N(c4ccc(-c5cccc6oc7ccccc7c56)cc4)c4cccc(-c5ccc6c(c5)-c5cccc7cccc(c57)S6)c4)cccc32)cc1]

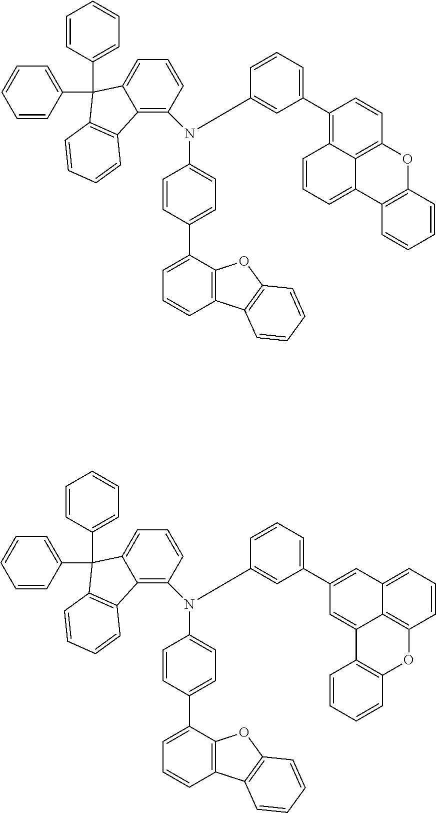 [SMILES: c1ccc(C2(c3ccccc3)c3ccccc3-c3c(N(c4ccc(-c5cccc6c5oc5ccccc56)cc4)c4cccc(-c5ccc6c7c(cccc57)-c5ccccc5O6)c4)cccc32)cc1 ; c1ccc(C2(c3ccccc3)c3ccccc3-c3c(N(c4ccc(-c5cccc6c5oc5ccccc56)cc4)c4cccc(-c5cc6c7c(cccc7c5)Oc5ccccc5-6)c4)cccc32)cc1]

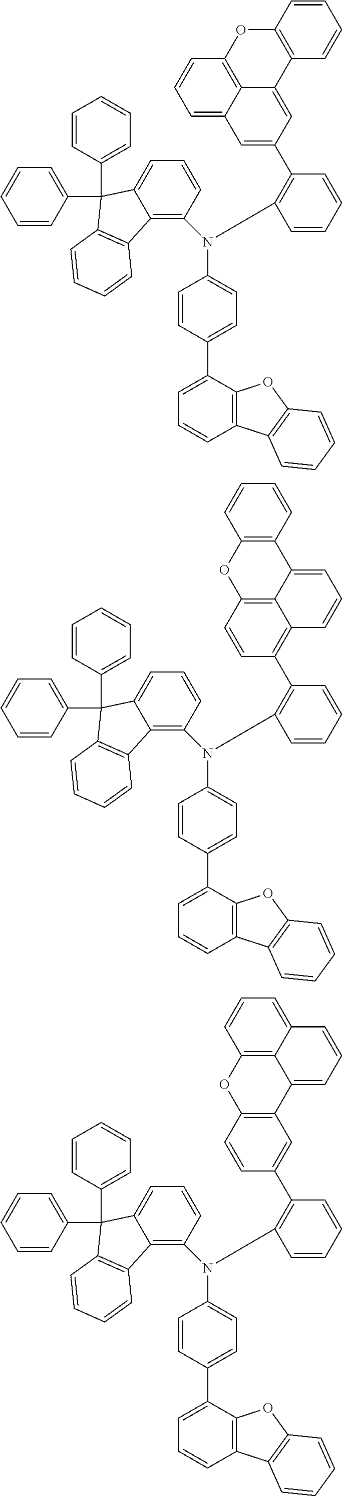

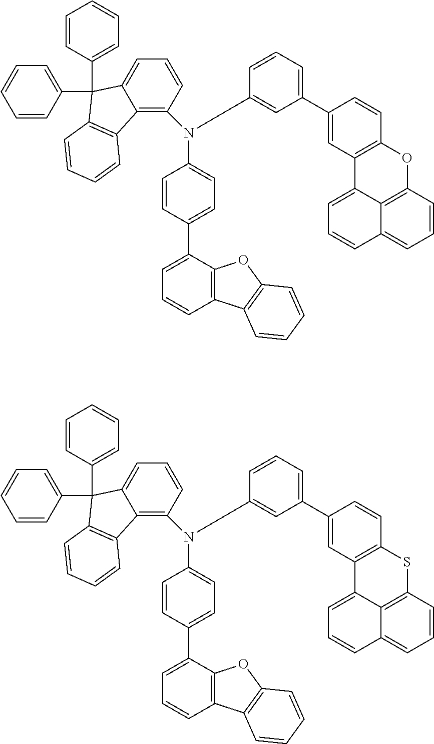 [SMILES: c1ccc(C2(c3ccccc3)c3ccccc3-c3c(N(c4ccc(-c5cccc6c5oc5ccccc56)cc4)c4cccc(-c5ccc6c(c5)-c5cccc7cccc(c57)O6)c4)cccc32)cc1 ; c1ccc(C2(c3ccccc3)c3ccccc3-c3c(N(c4ccc(-c5cccc6c5oc5ccccc56)cc4)c4cccc(-c5ccc6c(c5)-c5cccc7cccc(c57)S6)c4)cccc32)cc1]

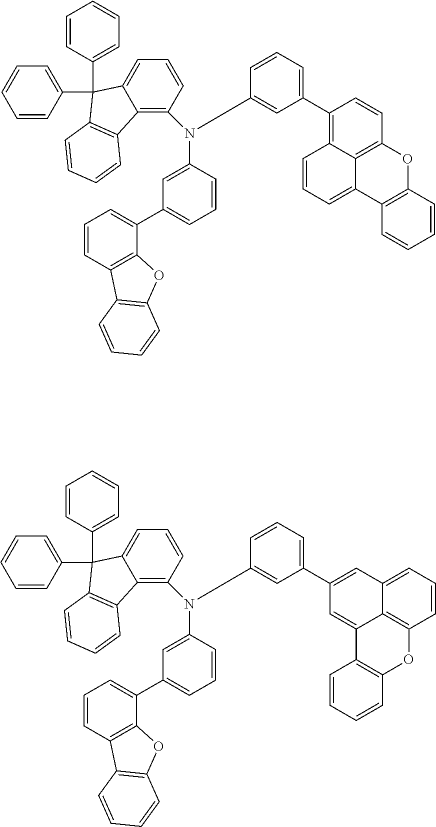 [SMILES: c1ccc(C2(c3ccccc3)c3ccccc3-c3c(N(c4cccc(-c5ccc6c7c(cccc57)-c5ccccc5O6)c4)c4cccc(-c5cccc6c5oc5ccccc56)c4)cccc32)cc1 ; c1ccc(C2(c3ccccc3)c3ccccc3-c3c(N(c4cccc(-c5cc6c7c(cccc7c5)Oc5ccccc5-6)c4)c4cccc(-c5cccc6c5oc5ccccc56)c4)cccc32)cc1]

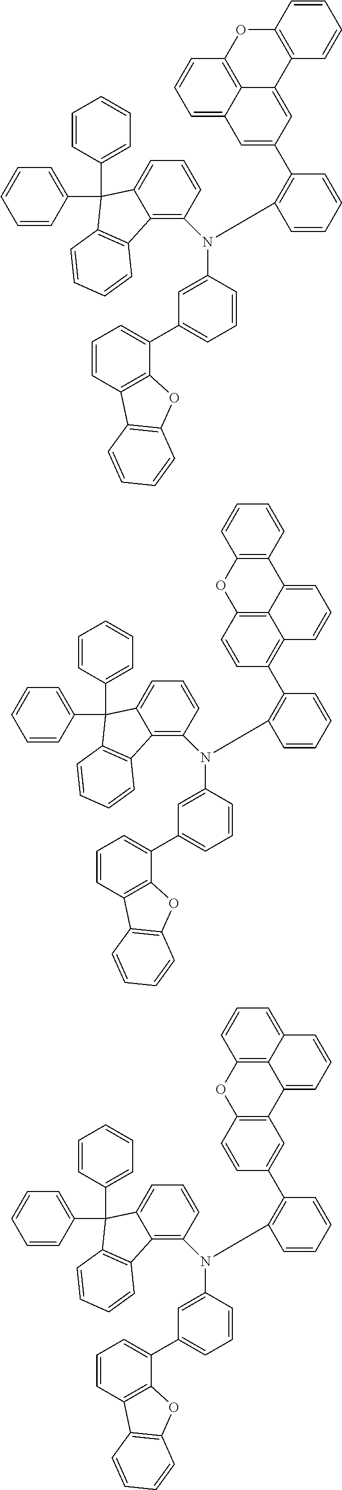 [SMILES: c1ccc(C2(c3ccccc3)c3ccccc3-c3c(N(c4cccc(-c5cccc6c5oc5ccccc56)c4)c4ccccc4-c4cc5c6c(cccc6c4)Oc4ccccc4-5)cccc32)cc1 ; c1ccc(C2(c3ccccc3)c3ccccc3-c3c(N(c4cccc(-c5cccc6c5oc5ccccc56)c4)c4ccccc4-c4ccc5c6c(cccc46)-c4ccccc4O5)cccc32)cc1 ; c1ccc(C2(c3ccccc3)c3ccccc3-c3c(N(c4cccc(-c5cccc6c5oc5ccccc56)c4)c4ccccc4-c4ccc5c(c4)-c4cccc6cccc(c46)O5)cccc32)cc1]

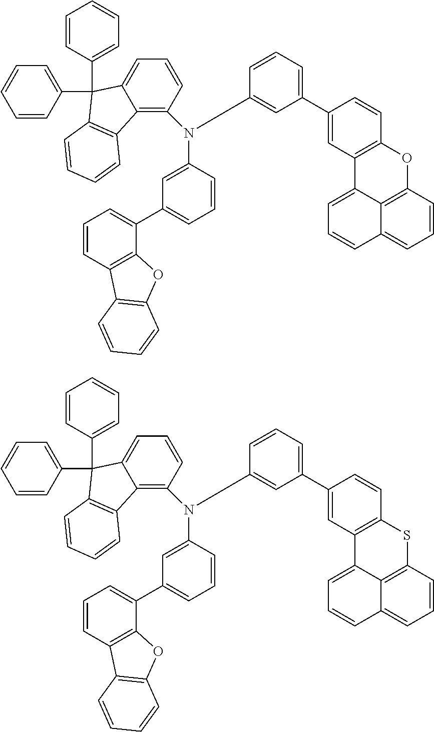

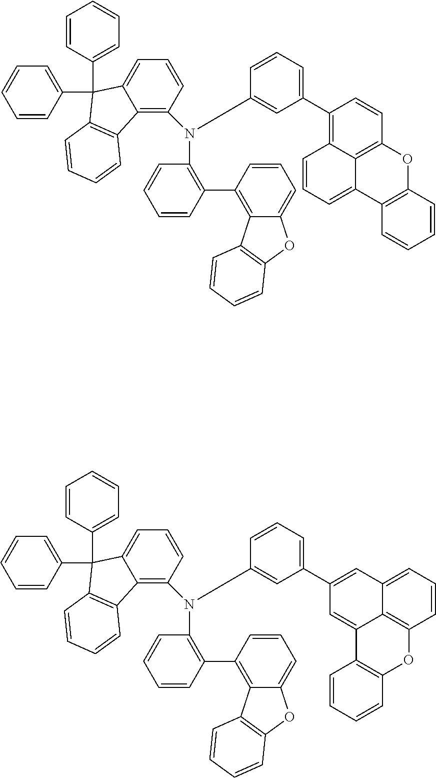 [SMILES: c1ccc(C2(c3ccccc3)c3ccccc3-c3c(N(c4cccc(-c5ccc6c7c(cccc57)-c5ccccc5O6)c4)c4ccccc4-c4cccc5oc6ccccc6c45)cccc32)cc1 ; c1ccc(C2(c3ccccc3)c3ccccc3-c3c(N(c4cccc(-c5cc6c7c(cccc7c5)Oc5ccccc5-6)c4)c4ccccc4-c4cccc5oc6ccccc6c45)cccc32)cc1]

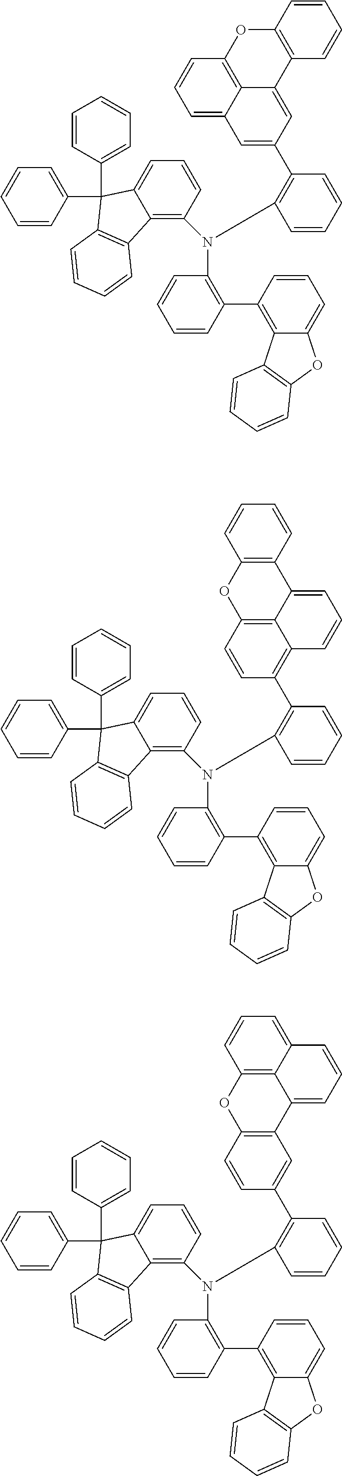 [SMILES: c1ccc(C2(c3ccccc3)c3ccccc3-c3c(N(c4ccccc4-c4cc5c6c(cccc6c4)Oc4ccccc4-5)c4ccccc4-c4cccc5oc6ccccc6c45)cccc32)cc1 ; c1ccc(C2(c3ccccc3)c3ccccc3-c3c(N(c4ccccc4-c4ccc5c6c(cccc46)-c4ccccc4O5)c4ccccc4-c4cccc5oc6ccccc6c45)cccc32)cc1 ; c1ccc(C2(c3ccccc3)c3ccccc3-c3c(N(c4ccccc4-c4ccc5c(c4)-c4cccc6cccc(c46)O5)c4ccccc4-c4cccc5oc6ccccc6c45)cccc32)cc1]

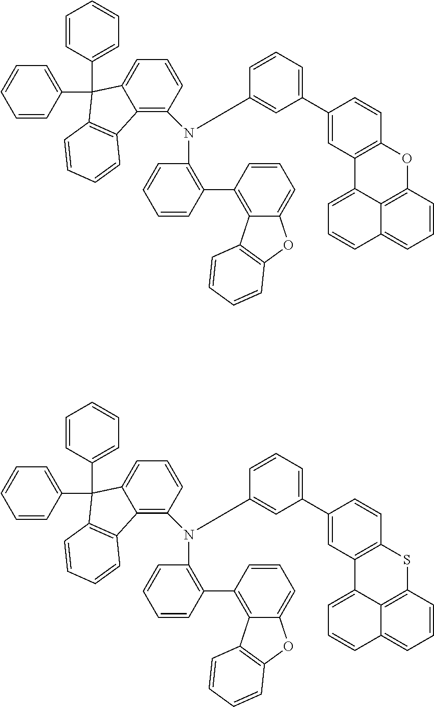 [SMILES: c1ccc(C2(c3ccccc3)c3ccccc3-c3c(N(c4cccc(-c5ccc6c(c5)-c5cccc7cccc(c57)O6)c4)c4ccccc4-c4cccc5oc6ccccc6c45)cccc32)cc1 ; c1ccc(C2(c3ccccc3)c3ccccc3-c3c(N(c4cccc(-c5ccc6c(c5)-c5cccc7cccc(c57)S6)c4)c4ccccc4-c4cccc5oc6ccccc6c45)cccc32)cc1]

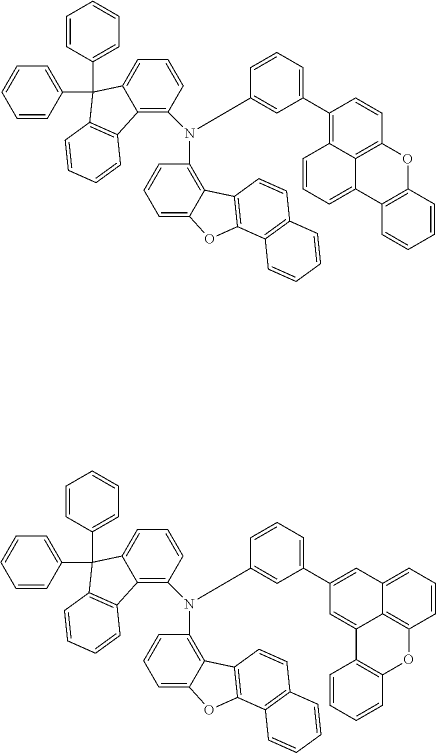 [SMILES: c1ccc(C2(c3ccccc3)c3ccccc3-c3c(N(c4cccc(-c5ccc6c7c(cccc57)-c5ccccc5O6)c4)c4cccc5oc6c7ccccc7ccc6c45)cccc32)cc1 ; c1ccc(C2(c3ccccc3)c3ccccc3-c3c(N(c4cccc(-c5cc6c7c(cccc7c5)Oc5ccccc5-6)c4)c4cccc5oc6c7ccccc7ccc6c45)cccc32)cc1]

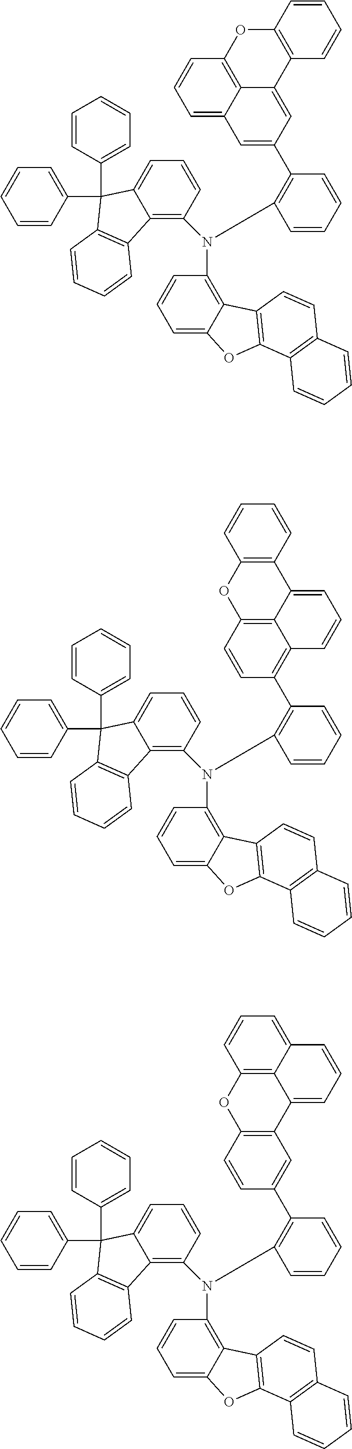 [SMILES: c1ccc(C2(c3ccccc3)c3ccccc3-c3c(N(c4ccccc4-c4cc5c6c(cccc6c4)Oc4ccccc4-5)c4cccc5oc6c7ccccc7ccc6c45)cccc32)cc1 ; c1ccc(C2(c3ccccc3)c3ccccc3-c3c(N(c4ccccc4-c4ccc5c6c(cccc46)-c4ccccc4O5)c4cccc5oc6c7ccccc7ccc6c45)cccc32)cc1 ; c1ccc(C2(c3ccccc3)c3ccccc3-c3c(N(c4ccccc4-c4ccc5c(c4)-c4cccc6cccc(c46)O5)c4cccc5oc6c7ccccc7ccc6c45)cccc32)cc1]

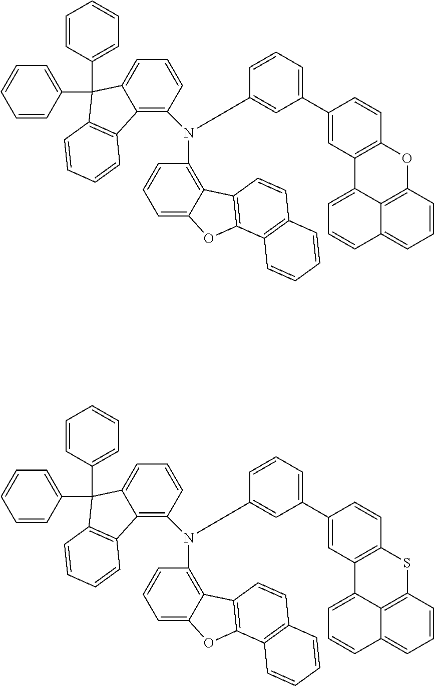 [SMILES: c1ccc(C2(c3ccccc3)c3ccccc3-c3c(N(c4cccc(-c5ccc6c(c5)-c5cccc7cccc(c57)O6)c4)c4cccc5oc6c7ccccc7ccc6c45)cccc32)cc1 ; c1ccc(C2(c3ccccc3)c3ccccc3-c3c(N(c4cccc(-c5ccc6c(c5)-c5cccc7cccc(c57)S6)c4)c4cccc5oc6c7ccccc7ccc6c45)cccc32)cc1]

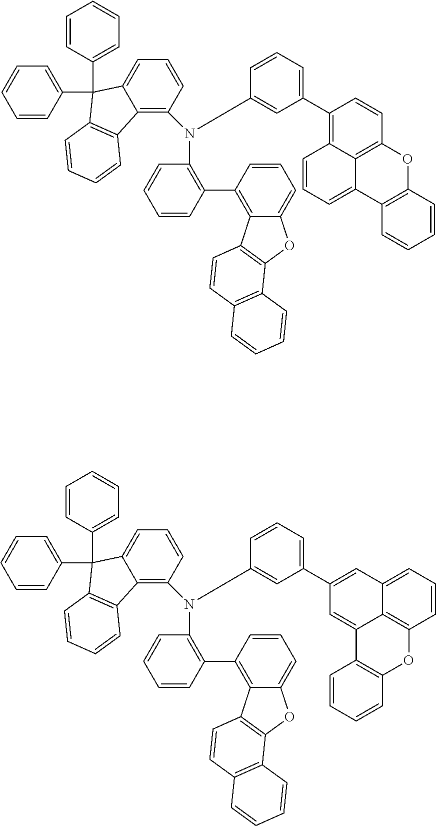 [SMILES: c1ccc(C2(c3ccccc3)c3ccccc3-c3c(N(c4cccc(-c5ccc6c7c(cccc57)-c5ccccc5O6)c4)c4ccccc4-c4cccc5oc6c7ccccc7ccc6c45)cccc32)cc1 ; c1ccc(C2(c3ccccc3)c3ccccc3-c3c(N(c4cccc(-c5cc6c7c(cccc7c5)Oc5ccccc5-6)c4)c4ccccc4-c4cccc5oc6c7ccccc7ccc6c45)cccc32)cc1]

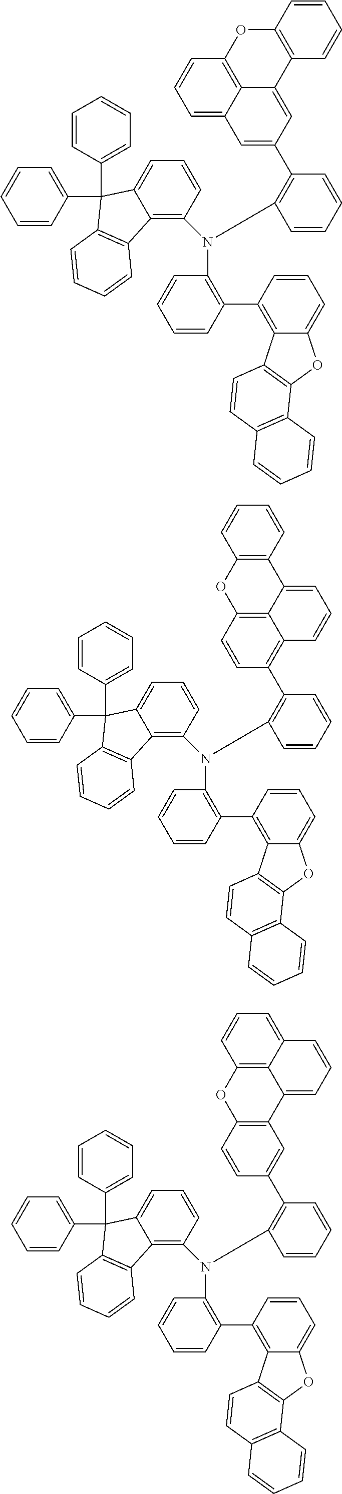

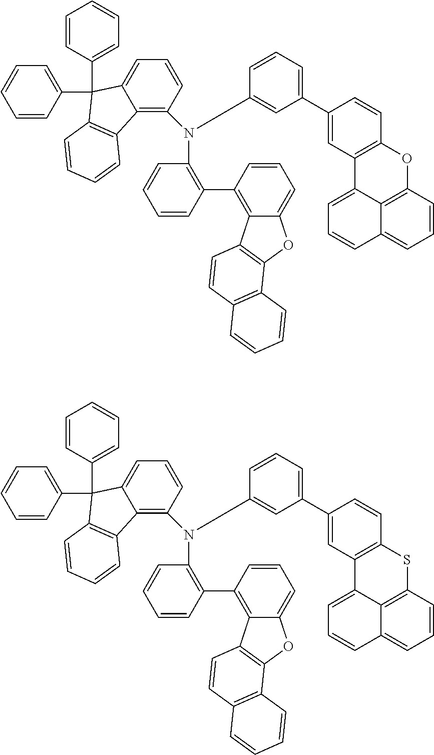 [SMILES: c1ccc(C2(c3ccccc3)c3ccccc3-c3c(N(c4cccc(-c5ccc6c(c5)-c5cccc7cccc(c57)O6)c4)c4ccccc4-c4cccc5oc6c7ccccc7ccc6c45)cccc32)cc1 ; c1ccc(C2(c3ccccc3)c3ccccc3-c3c(N(c4cccc(-c5ccc6c(c5)-c5cccc7cccc(c57)S6)c4)c4ccccc4-c4cccc5oc6c7ccccc7ccc6c45)cccc32)cc1]

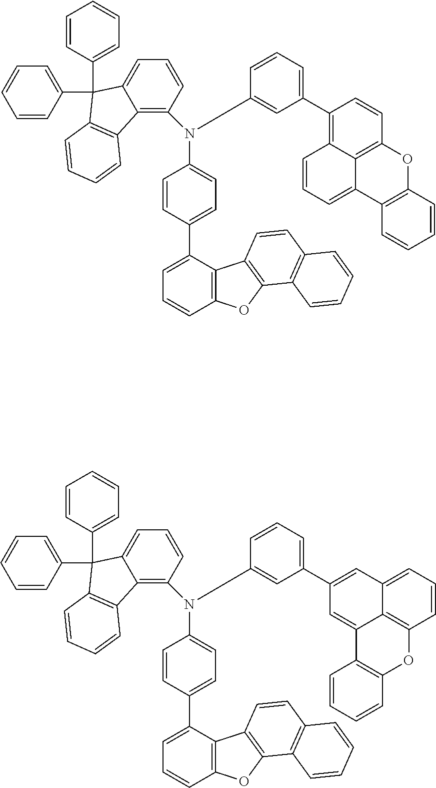 [SMILES: c1ccc(C2(c3ccccc3)c3ccccc3-c3c(N(c4ccc(-c5cccc6oc7c8ccccc8ccc7c56)cc4)c4cccc(-c5ccc6c7c(cccc57)-c5ccccc5O6)c4)cccc32)cc1 ; c1ccc(C2(c3ccccc3)c3ccccc3-c3c(N(c4ccc(-c5cccc6oc7c8ccccc8ccc7c56)cc4)c4cccc(-c5cc6c7c(cccc7c5)Oc5ccccc5-6)c4)cccc32)cc1]

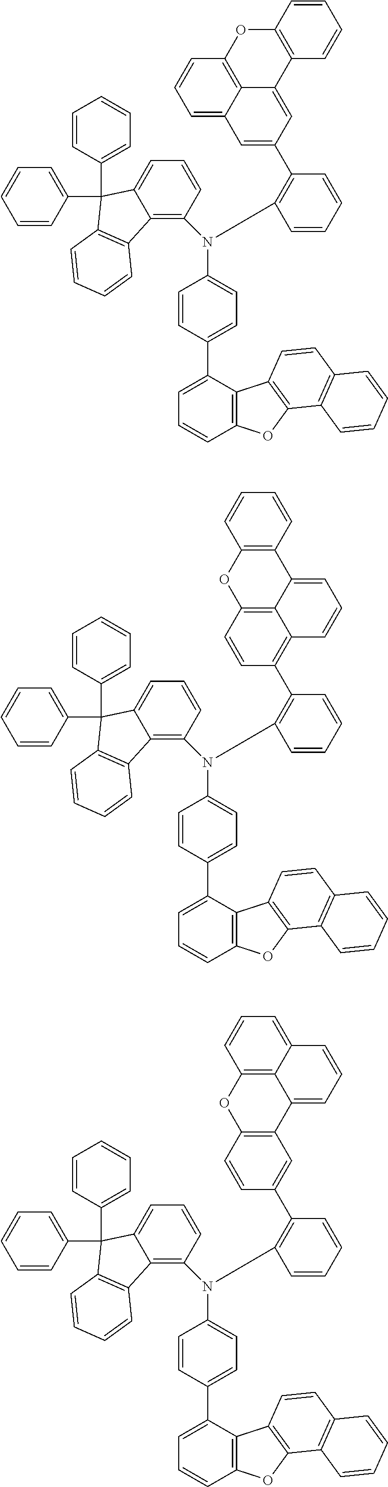 [SMILES: c1ccc(C2(c3ccccc3)c3ccccc3-c3c(N(c4ccc(-c5cccc6oc7c8ccccc8ccc7c56)cc4)c4ccccc4-c4cc5c6c(cccc6c4)Oc4ccccc4-5)cccc32)cc1 ; c1ccc(C2(c3ccccc3)c3ccccc3-c3c(N(c4ccc(-c5cccc6oc7c8ccccc8ccc7c56)cc4)c4ccccc4-c4ccc5c6c(cccc46)-c4ccccc4O5)cccc32)cc1 ; c1ccc(C2(c3ccccc3)c3ccccc3-c3c(N(c4ccc(-c5cccc6oc7c8ccccc8ccc7c56)cc4)c4ccccc4-c4ccc5c(c4)-c4cccc6cccc(c46)O5)cccc32)cc1]

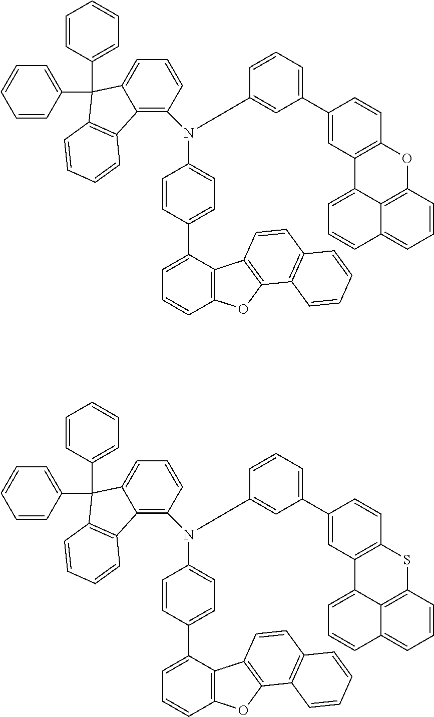 [SMILES: c1ccc(C2(c3ccccc3)c3ccccc3-c3c(N(c4ccc(-c5cccc6oc7c8ccccc8ccc7c56)cc4)c4cccc(-c5ccc6c(c5)-c5cccc7cccc(c57)O6)c4)cccc32)cc1 ; c1ccc(C2(c3ccccc3)c3ccccc3-c3c(N(c4ccc(-c5cccc6oc7c8ccccc8ccc7c56)cc4)c4cccc(-c5ccc6c(c5)-c5cccc7cccc(c57)S6)c4)cccc32)cc1]

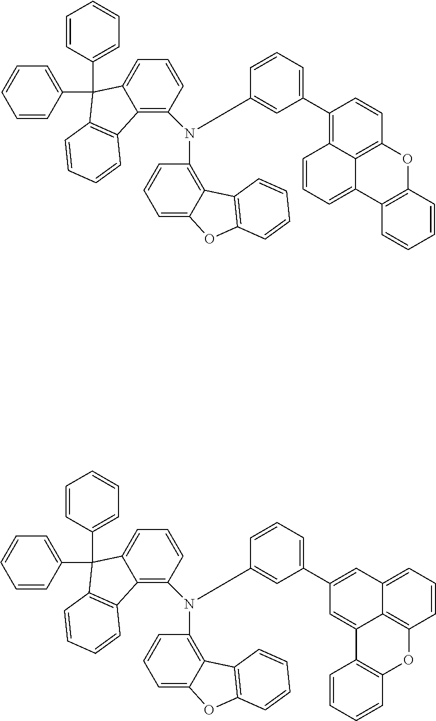 [SMILES: c1ccc(C2(c3ccccc3)c3ccccc3-c3c(N(c4cccc(-c5ccc6c7c(cccc57)-c5ccccc5O6)c4)c4cccc5oc6ccccc6c45)cccc32)cc1 ; c1ccc(C2(c3ccccc3)c3ccccc3-c3c(N(c4cccc(-c5cc6c7c(cccc7c5)Oc5ccccc5-6)c4)c4cccc5oc6ccccc6c45)cccc32)cc1]

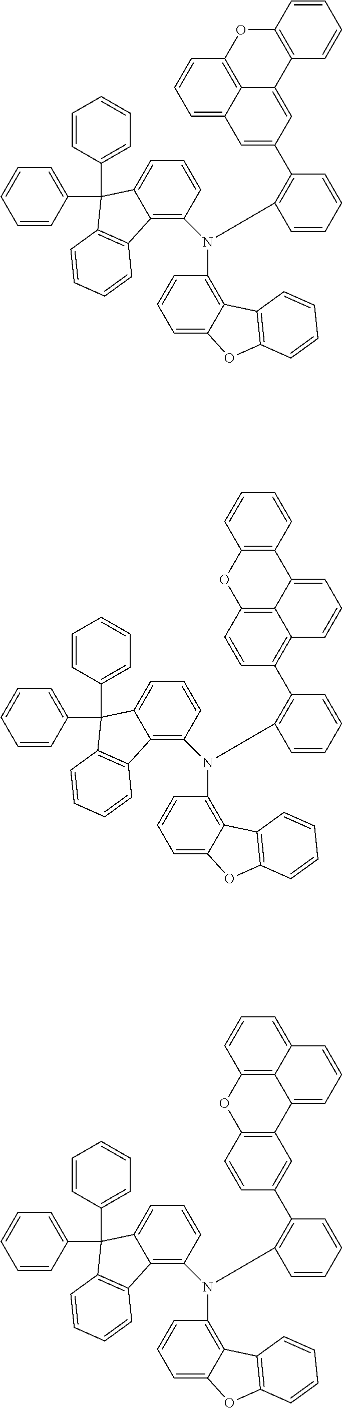 [SMILES: c1ccc(C2(c3ccccc3)c3ccccc3-c3c(N(c4ccccc4-c4cc5c6c(cccc6c4)Oc4ccccc4-5)c4cccc5oc6ccccc6c45)cccc32)cc1 ; c1ccc(C2(c3ccccc3)c3ccccc3-c3c(N(c4ccccc4-c4ccc5c6c(cccc46)-c4ccccc4O5)c4cccc5oc6ccccc6c45)cccc32)cc1 ; c1ccc(C2(c3ccccc3)c3ccccc3-c3c(N(c4ccccc4-c4ccc5c(c4)-c4cccc6cccc(c46)O5)c4cccc5oc6ccccc6c45)cccc32)cc1]

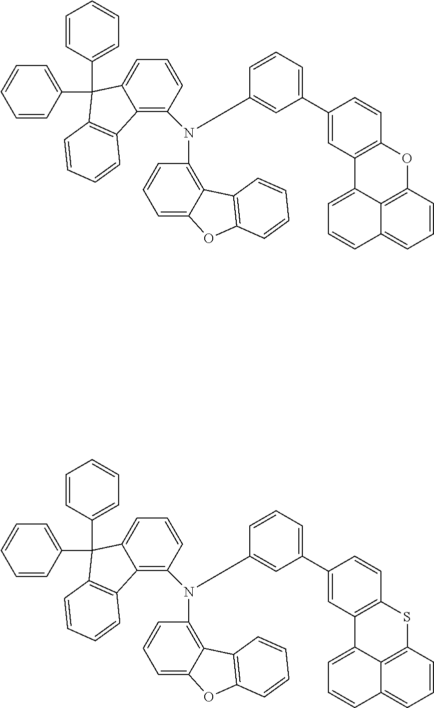 [SMILES: c1ccc(C2(c3ccccc3)c3ccccc3-c3c(N(c4cccc(-c5ccc6c(c5)-c5cccc7cccc(c57)O6)c4)c4cccc5oc6ccccc6c45)cccc32)cc1 ; c1ccc(C2(c3ccccc3)c3ccccc3-c3c(N(c4cccc(-c5ccc6c(c5)-c5cccc7cccc(c57)S6)c4)c4cccc5oc6ccccc6c45)cccc32)cc1]

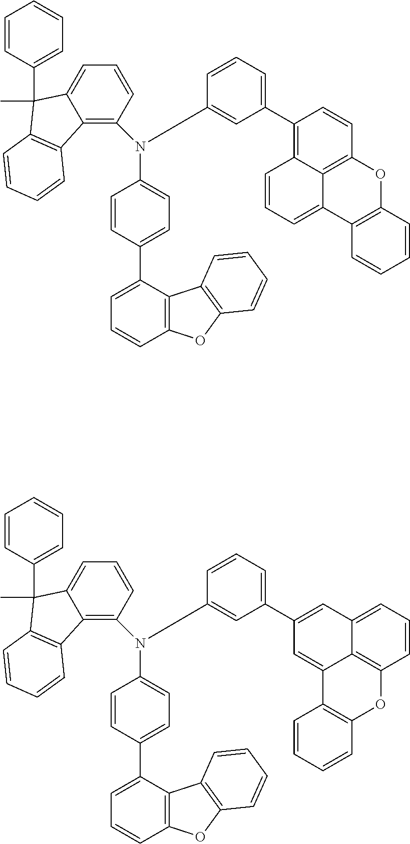 [SMILES: CC1(c2ccccc2)c2ccccc2-c2c(N(c3ccc(-c4cccc5oc6ccccc6c45)cc3)c3cccc(-c4ccc5c6c(cccc46)-c4ccccc4O5)c3)cccc21 ; CC1(c2ccccc2)c2ccccc2-c2c(N(c3ccc(-c4cccc5oc6ccccc6c45)cc3)c3cccc(-c4cc5c6c(cccc6c4)Oc4ccccc4-5)c3)cccc21]

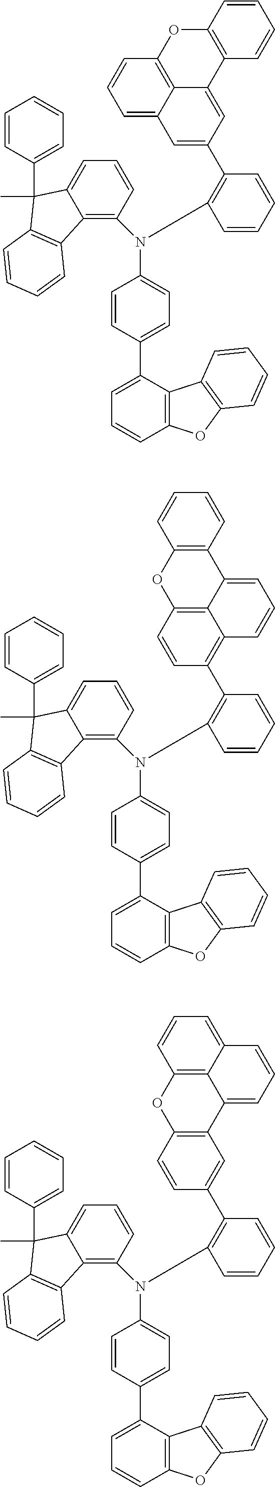 [SMILES: CC1(c2ccccc2)c2ccccc2-c2c(N(c3ccc(-c4cccc5oc6ccccc6c45)cc3)c3ccccc3-c3cc4c5c(cccc5c3)Oc3ccccc3-4)cccc21 ; CC1(c2ccccc2)c2ccccc2-c2c(N(c3ccc(-c4cccc5oc6ccccc6c45)cc3)c3ccccc3-c3ccc4c5c(cccc35)-c3ccccc3O4)cccc21 ; CC1(c2ccccc2)c2ccccc2-c2c(N(c3ccc(-c4cccc5oc6ccccc6c45)cc3)c3ccccc3-c3ccc4c(c3)-c3cccc5cccc(c35)O4)cccc21]

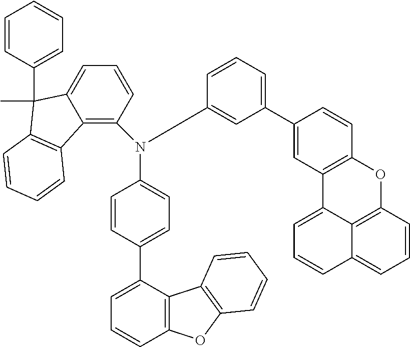 [SMILES: CC1(c2ccccc2)c2ccccc2-c2c(N(c3ccc(-c4cccc5oc6ccccc6c45)cc3)c3cccc(-c4ccc5c(c4)-c4cccc6cccc(c46)O5)c3)cccc21]

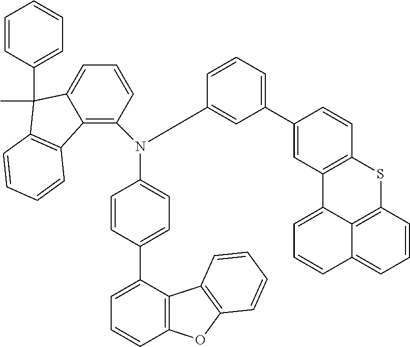 [SMILES: CC1(c2ccccc2)c2ccccc2-c2c(N(c3ccc(-c4cccc5oc6ccccc6c45)cc3)c3cccc(-c4ccc5c(c4)-c4cccc6cccc(c46)S5)c3)cccc21]

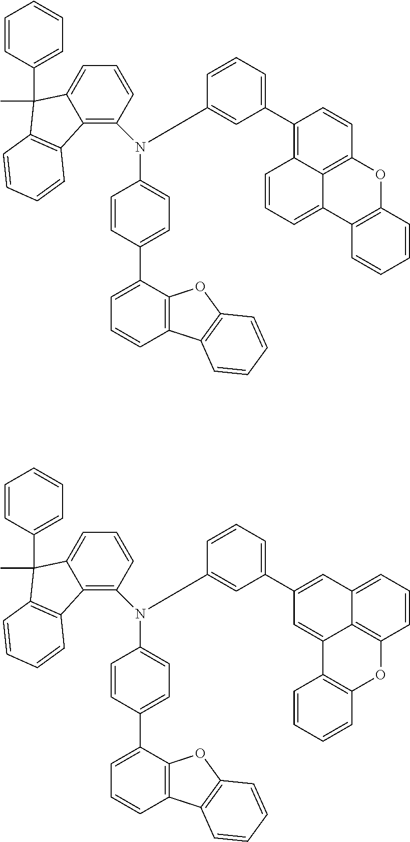 [SMILES: CC1(c2ccccc2)c2ccccc2-c2c(N(c3ccc(-c4cccc5c4oc4ccccc45)cc3)c3cccc(-c4ccc5c6c(cccc46)-c4ccccc4O5)c3)cccc21 ; CC1(c2ccccc2)c2ccccc2-c2c(N(c3ccc(-c4cccc5c4oc4ccccc45)cc3)c3cccc(-c4cc5c6c(cccc6c4)Oc4ccccc4-5)c3)cccc21]

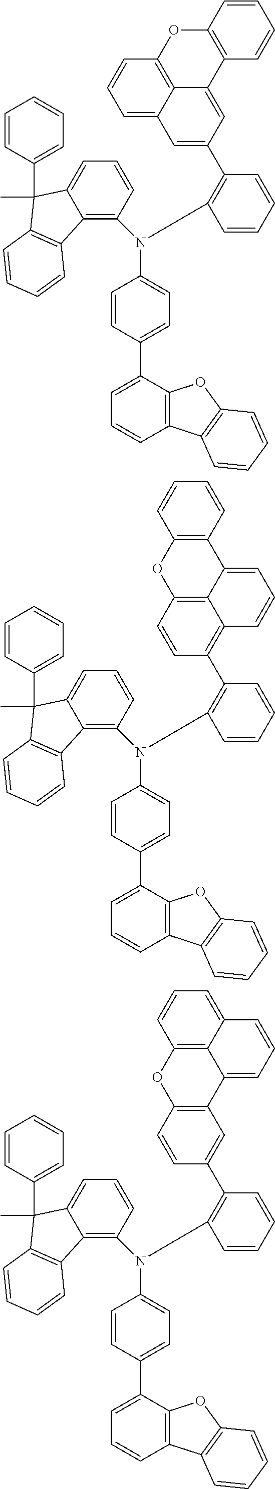

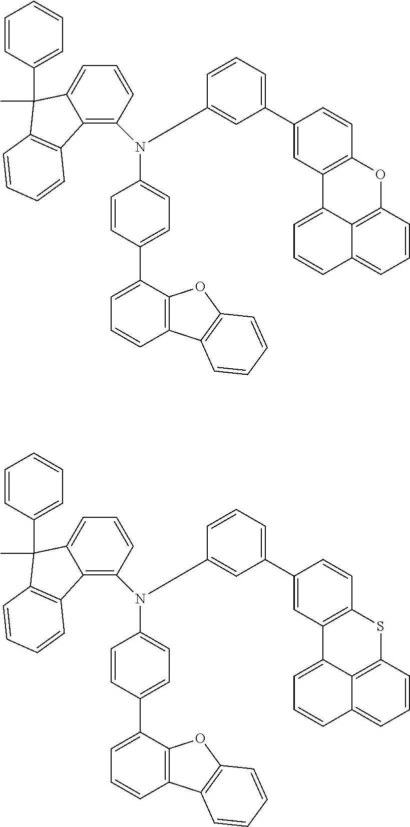 [SMILES: CC1(c2ccccc2)c2ccccc2-c2c(N(c3ccc(-c4cccc5c4oc4ccccc45)cc3)c3cccc(-c4ccc5c(c4)-c4cccc6cccc(c46)O5)c3)cccc21 ; CC1(c2ccccc2)c2ccccc2-c2c(N(c3ccc(-c4cccc5c4oc4ccccc45)cc3)c3cccc(-c4ccc5c(c4)-c4cccc6cccc(c46)S5)c3)cccc21]

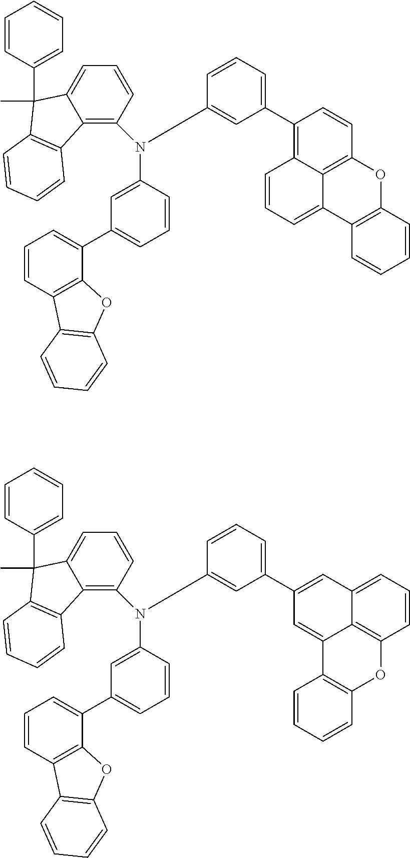 [SMILES: CC1(c2ccccc2)c2ccccc2-c2c(N(c3cccc(-c4ccc5c6c(cccc46)-c4ccccc4O5)c3)c3cccc(-c4cccc5c4oc4ccccc45)c3)cccc21 ; CC1(c2ccccc2)c2ccccc2-c2c(N(c3cccc(-c4cc5c6c(cccc6c4)Oc4ccccc4-5)c3)c3cccc(-c4cccc5c4oc4ccccc45)c3)cccc21]

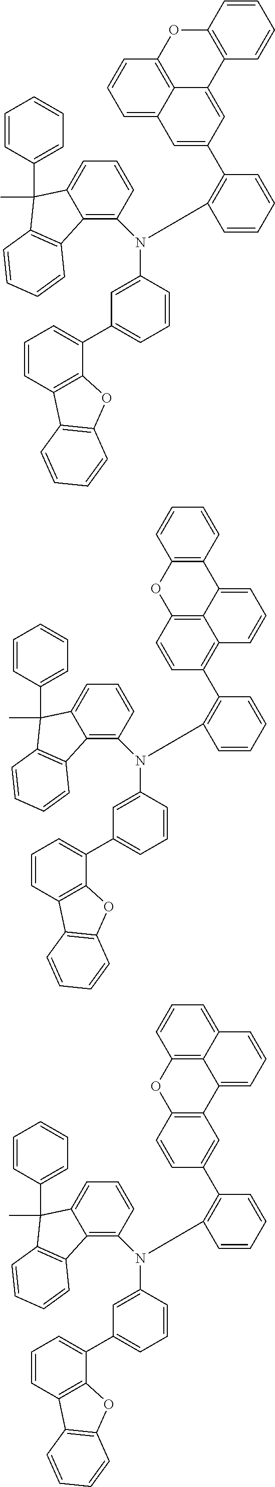 [SMILES: CC1(c2ccccc2)c2ccccc2-c2c(N(c3cccc(-c4cccc5c4oc4ccccc45)c3)c3ccccc3-c3cc4c5c(cccc5c3)Oc3ccccc3-4)cccc21 ; CC1(c2ccccc2)c2ccccc2-c2c(N(c3cccc(-c4cccc5c4oc4ccccc45)c3)c3ccccc3-c3ccc4c5c(cccc35)-c3ccccc3O4)cccc21 ; CC1(c2ccccc2)c2ccccc2-c2c(N(c3cccc(-c4cccc5c4oc4ccccc45)c3)c3ccccc3-c3ccc4c(c3)-c3cccc5cccc(c35)O4)cccc21]

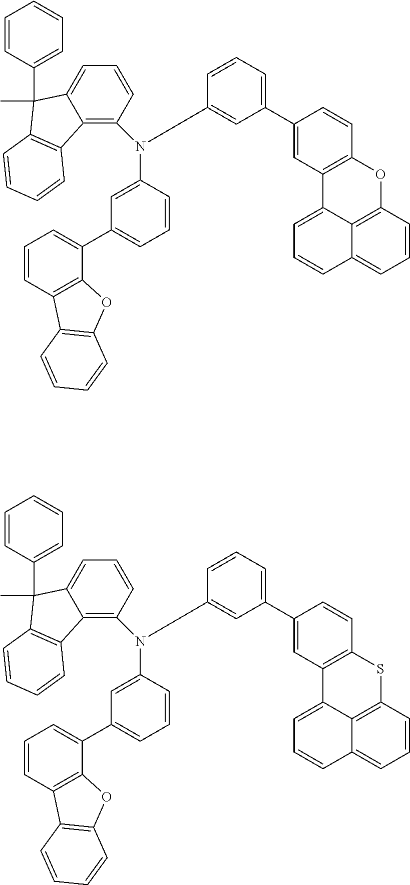 [SMILES: CC1(c2ccccc2)c2ccccc2-c2c(N(c3cccc(-c4ccc5c(c4)-c4cccc6cccc(c46)O5)c3)c3cccc(-c4cccc5c4oc4ccccc45)c3)cccc21 ; CC1(c2ccccc2)c2ccccc2-c2c(N(c3cccc(-c4ccc5c(c4)-c4cccc6cccc(c46)S5)c3)c3cccc(-c4cccc5c4oc4ccccc45)c3)cccc21]

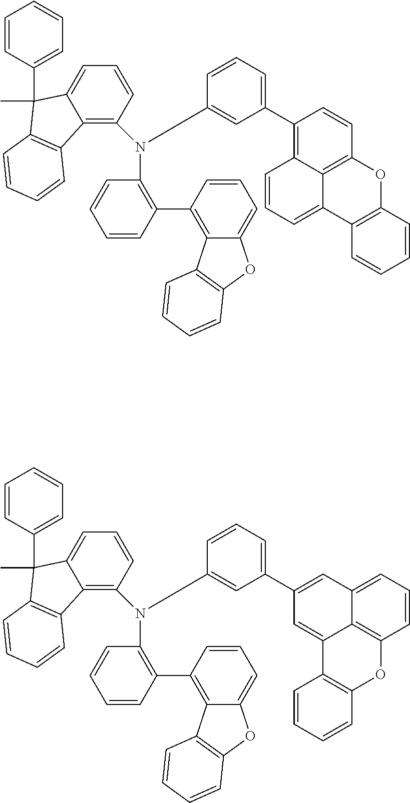 [SMILES: CC1(c2ccccc2)c2ccccc2-c2c(N(c3cccc(-c4ccc5c6c(cccc46)-c4ccccc4O5)c3)c3ccccc3-c3cccc4oc5ccccc5c34)cccc21 ; CC1(c2ccccc2)c2ccccc2-c2c(N(c3cccc(-c4cc5c6c(cccc6c4)Oc4ccccc4-5)c3)c3ccccc3-c3cccc4oc5ccccc5c34)cccc21]

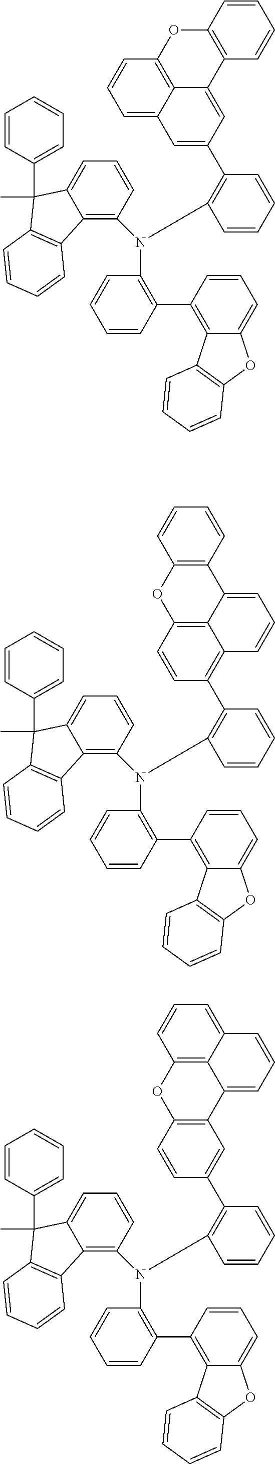

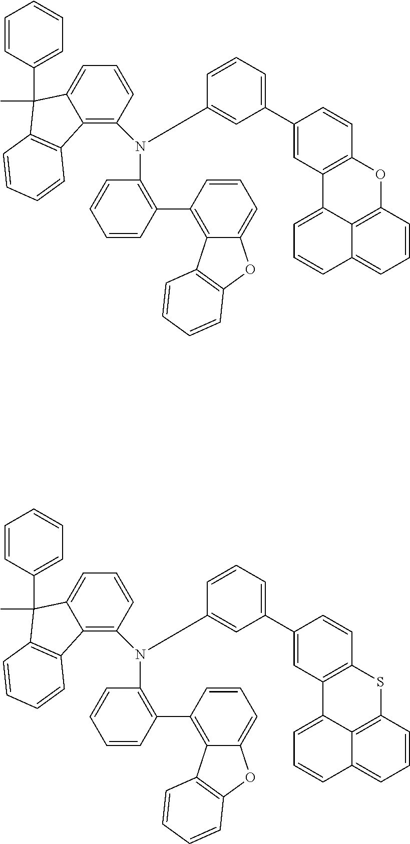 [SMILES: CC1(c2ccccc2)c2ccccc2-c2c(N(c3cccc(-c4ccc5c(c4)-c4cccc6cccc(c46)O5)c3)c3ccccc3-c3cccc4oc5ccccc5c34)cccc21 ; CC1(c2ccccc2)c2ccccc2-c2c(N(c3cccc(-c4ccc5c(c4)-c4cccc6cccc(c46)S5)c3)c3ccccc3-c3cccc4oc5ccccc5c34)cccc21]

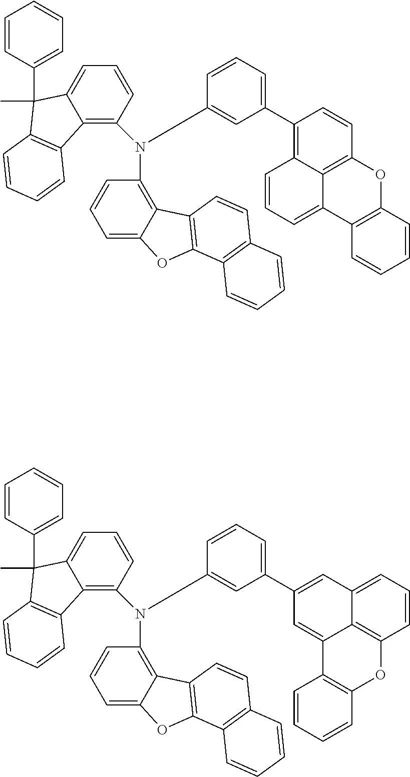 [SMILES: CC1(c2ccccc2)c2ccccc2-c2c(N(c3cccc(-c4ccc5c6c(cccc46)-c4ccccc4O5)c3)c3cccc4oc5c6ccccc6ccc5c34)cccc21 ; CC1(c2ccccc2)c2ccccc2-c2c(N(c3cccc(-c4cc5c6c(cccc6c4)Oc4ccccc4-5)c3)c3cccc4oc5c6ccccc6ccc5c34)cccc21]

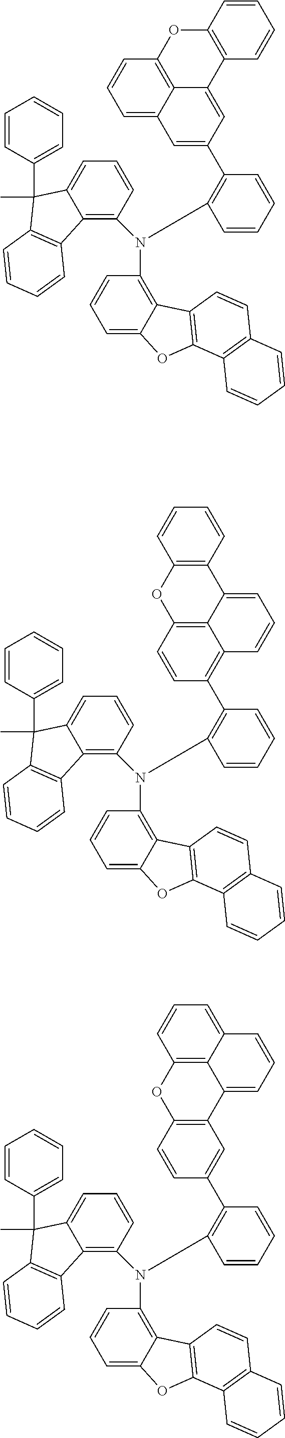 [SMILES: CC1(c2ccccc2)c2ccccc2-c2c(N(c3ccccc3-c3cc4c5c(cccc5c3)Oc3ccccc3-4)c3cccc4oc5c6ccccc6ccc5c34)cccc21 ; CC1(c2ccccc2)c2ccccc2-c2c(N(c3ccccc3-c3ccc4c5c(cccc35)-c3ccccc3O4)c3cccc4oc5c6ccccc6ccc5c34)cccc21 ; CC1(c2ccccc2)c2ccccc2-c2c(N(c3ccccc3-c3ccc4c(c3)-c3cccc5cccc(c35)O4)c3cccc4oc5c6ccccc6ccc5c34)cccc21]

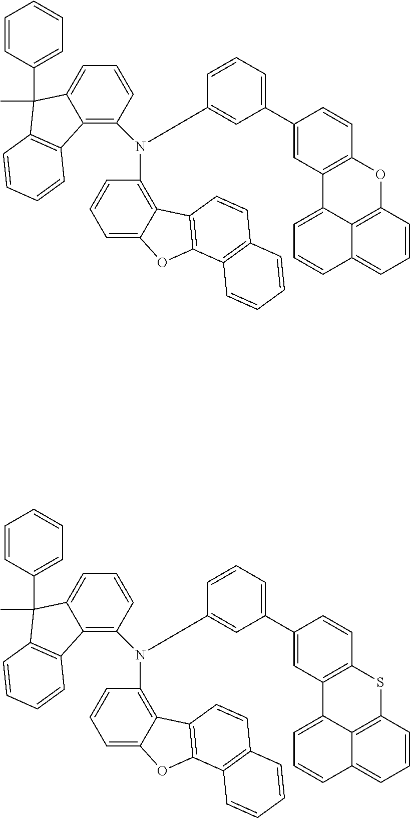 [SMILES: CC1(c2ccccc2)c2ccccc2-c2c(N(c3cccc(-c4ccc5c(c4)-c4cccc6cccc(c46)O5)c3)c3cccc4oc5c6ccccc6ccc5c34)cccc21 ; CC1(c2ccccc2)c2ccccc2-c2c(N(c3cccc(-c4ccc5c(c4)-c4cccc6cccc(c46)S5)c3)c3cccc4oc5c6ccccc6ccc5c34)cccc21]

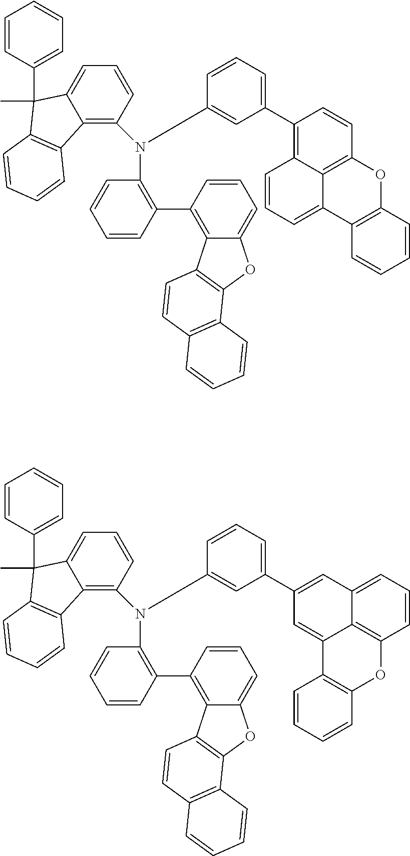 [SMILES: CC1(c2ccccc2)c2ccccc2-c2c(N(c3cccc(-c4ccc5c6c(cccc46)-c4ccccc4O5)c3)c3ccccc3-c3cccc4oc5c6ccccc6ccc5c34)cccc21 ; CC1(c2ccccc2)c2ccccc2-c2c(N(c3cccc(-c4cc5c6c(cccc6c4)Oc4ccccc4-5)c3)c3ccccc3-c3cccc4oc5c6ccccc6ccc5c34)cccc21]

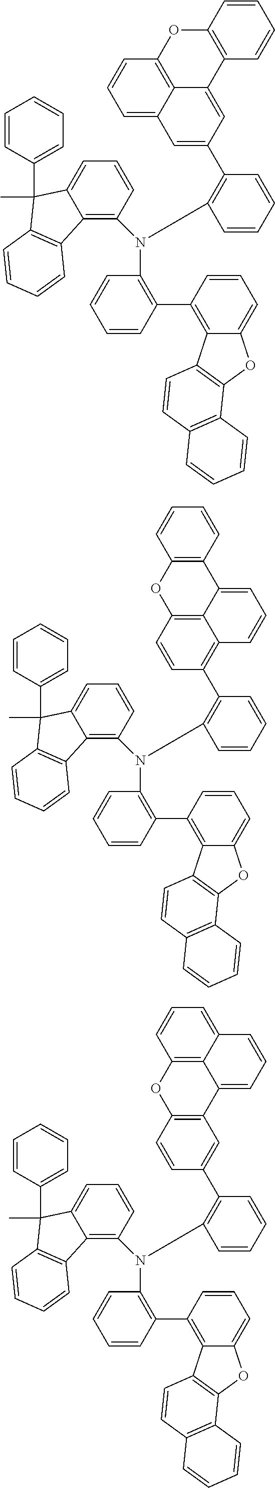

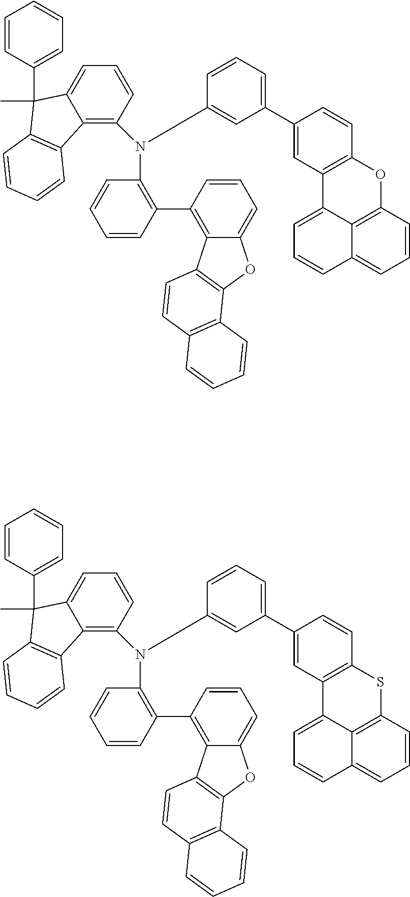 [SMILES: CC1(c2ccccc2)c2ccccc2-c2c(N(c3cccc(-c4ccc5c(c4)-c4cccc6cccc(c46)O5)c3)c3ccccc3-c3cccc4oc5c6ccccc6ccc5c34)cccc21 ; CC1(c2ccccc2)c2ccccc2-c2c(N(c3cccc(-c4ccc5c(c4)-c4cccc6cccc(c46)S5)c3)c3ccccc3-c3cccc4oc5c6ccccc6ccc5c34)cccc21]

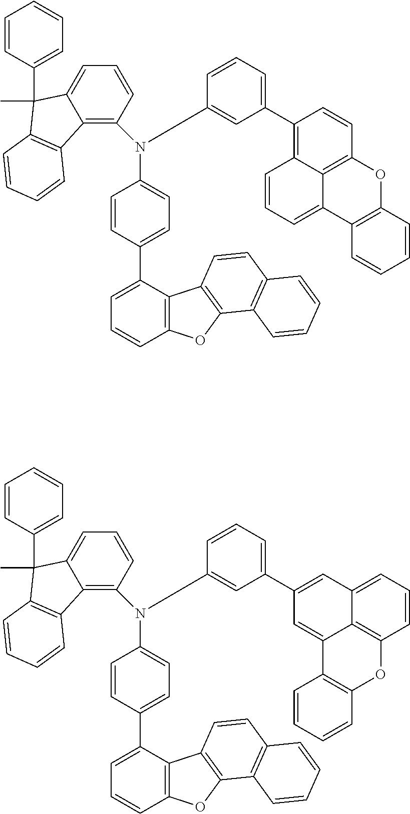 [SMILES: CC1(c2ccccc2)c2ccccc2-c2c(N(c3ccc(-c4cccc5oc6c7ccccc7ccc6c45)cc3)c3cccc(-c4ccc5c6c(cccc46)-c4ccccc4O5)c3)cccc21 ; CC1(c2ccccc2)c2ccccc2-c2c(N(c3ccc(-c4cccc5oc6c7ccccc7ccc6c45)cc3)c3cccc(-c4cc5c6c(cccc6c4)Oc4ccccc4-5)c3)cccc21]

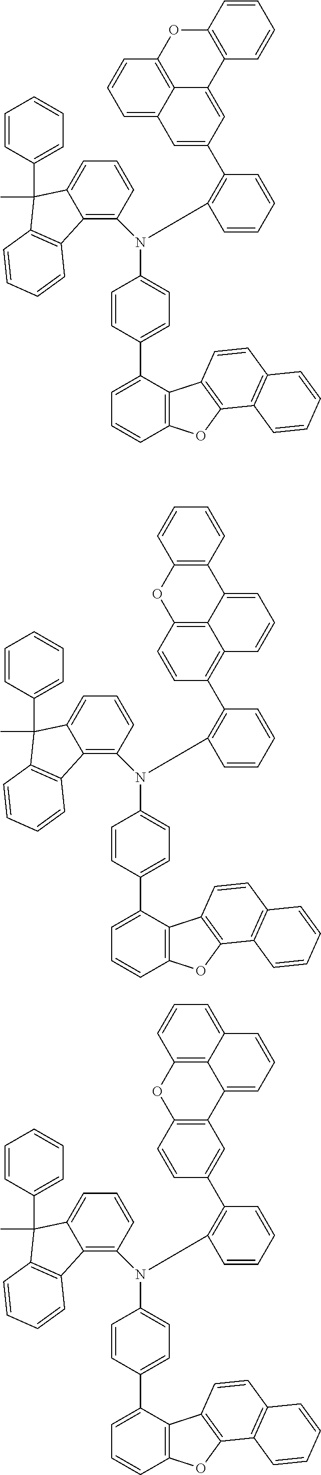

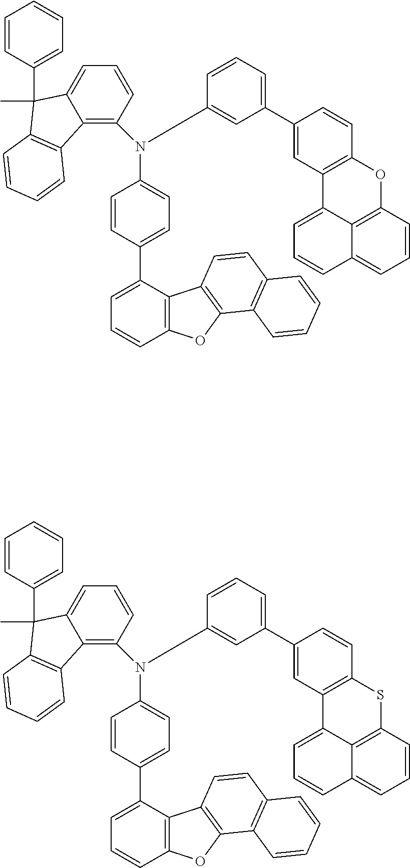 [SMILES: CC1(c2ccccc2)c2ccccc2-c2c(N(c3ccc(-c4cccc5oc6c7ccccc7ccc6c45)cc3)c3cccc(-c4ccc5c(c4)-c4cccc6cccc(c46)O5)c3)cccc21 ; CC1(c2ccccc2)c2ccccc2-c2c(N(c3ccc(-c4cccc5oc6c7ccccc7ccc6c45)cc3)c3cccc(-c4ccc5c(c4)-c4cccc6cccc(c46)S5)c3)cccc21]

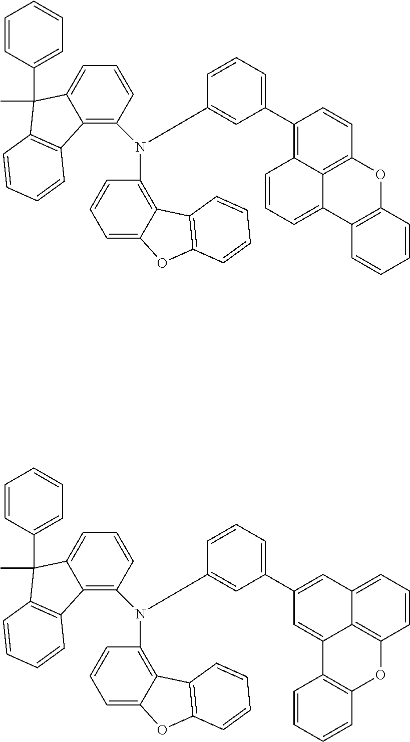 [SMILES: CC1(c2ccccc2)c2ccccc2-c2c(N(c3cccc(-c4ccc5c6c(cccc46)-c4ccccc4O5)c3)c3cccc4oc5ccccc5c34)cccc21 ; CC1(c2ccccc2)c2ccccc2-c2c(N(c3cccc(-c4cc5c6c(cccc6c4)Oc4ccccc4-5)c3)c3cccc4oc5ccccc5c34)cccc21]

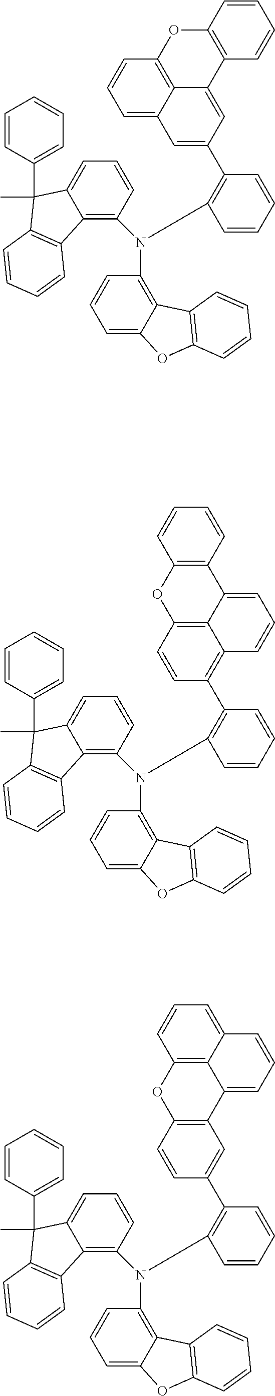 [SMILES: CC1(c2ccccc2)c2ccccc2-c2c(N(c3ccccc3-c3cc4c5c(cccc5c3)Oc3ccccc3-4)c3cccc4oc5ccccc5c34)cccc21 ; CC1(c2ccccc2)c2ccccc2-c2c(N(c3ccccc3-c3ccc4c5c(cccc35)-c3ccccc3O4)c3cccc4oc5ccccc5c34)cccc21 ; CC1(c2ccccc2)c2ccccc2-c2c(N(c3ccccc3-c3ccc4c(c3)-c3cccc5cccc(c35)O4)c3cccc4oc5ccccc5c34)cccc21]

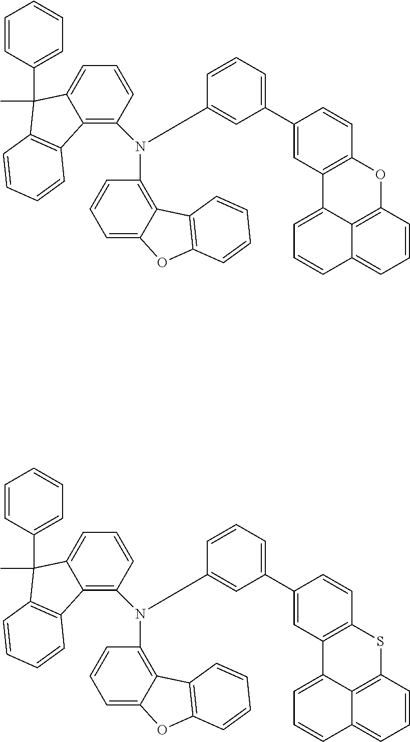 [SMILES: CC1(c2ccccc2)c2ccccc2-c2c(N(c3cccc(-c4ccc5c(c4)-c4cccc6cccc(c46)O5)c3)c3cccc4oc5ccccc5c34)cccc21 ; CC1(c2ccccc2)c2ccccc2-c2c(N(c3cccc(-c4ccc5c(c4)-c4cccc6cccc(c46)S5)c3)c3cccc4oc5ccccc5c34)cccc21]

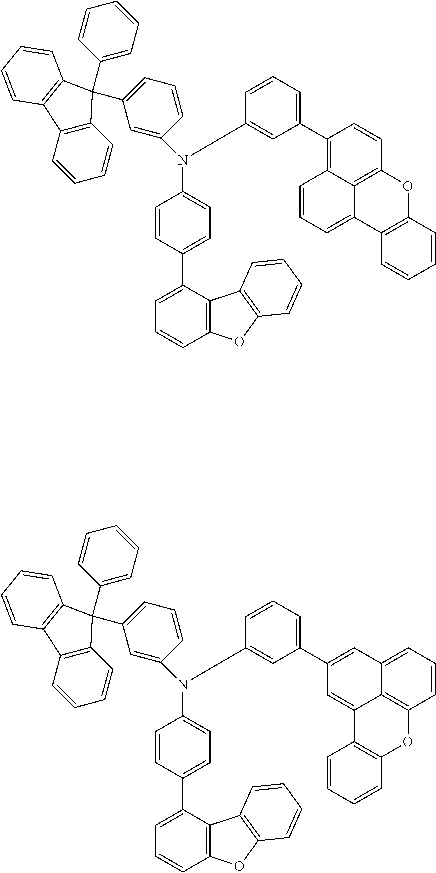 [SMILES: c1ccc(C2(c3cccc(N(c4ccc(-c5cccc6oc7ccccc7c56)cc4)c4cccc(-c5ccc6c7c(cccc57)-c5ccccc5O6)c4)c3)c3ccccc3-c3ccccc32)cc1 ; c1ccc(C2(c3cccc(N(c4ccc(-c5cccc6oc7ccccc7c56)cc4)c4cccc(-c5cc6c7c(cccc7c5)Oc5ccccc5-6)c4)c3)c3ccccc3-c3ccccc32)cc1]

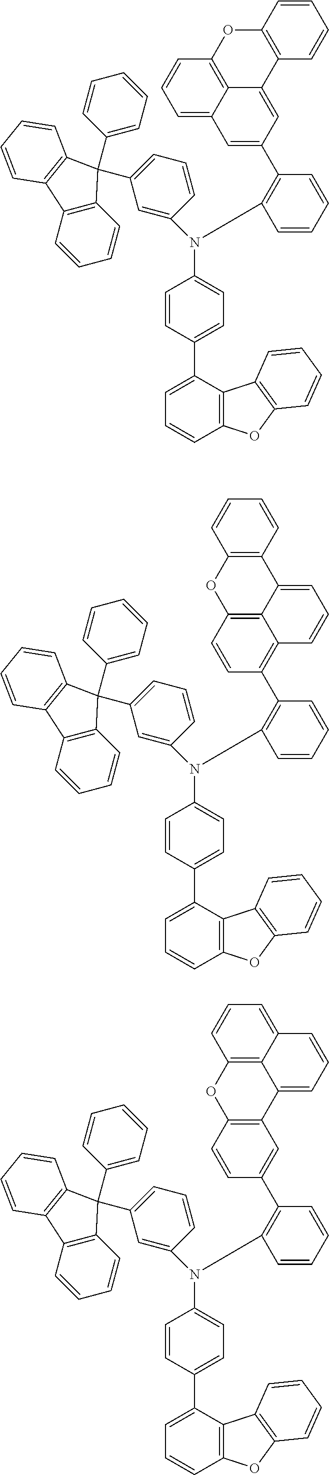

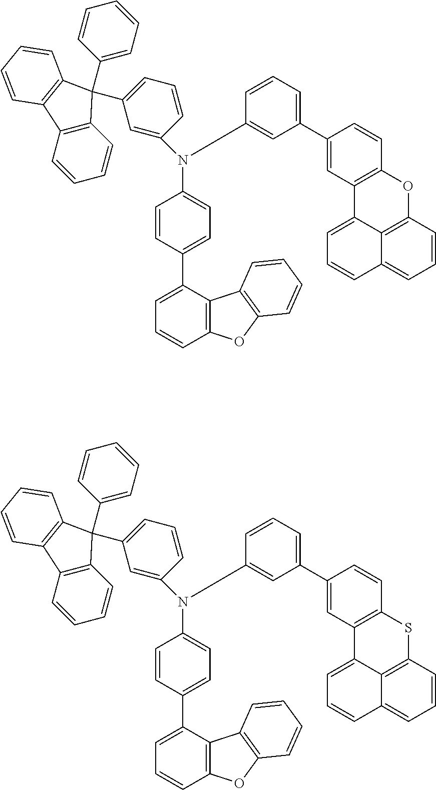 [SMILES: c1ccc(C2(c3cccc(N(c4ccc(-c5cccc6oc7ccccc7c56)cc4)c4cccc(-c5ccc6c(c5)-c5cccc7cccc(c57)O6)c4)c3)c3ccccc3-c3ccccc32)cc1 ; c1ccc(C2(c3cccc(N(c4ccc(-c5cccc6oc7ccccc7c56)cc4)c4cccc(-c5ccc6c(c5)-c5cccc7cccc(c57)S6)c4)c3)c3ccccc3-c3ccccc32)cc1]

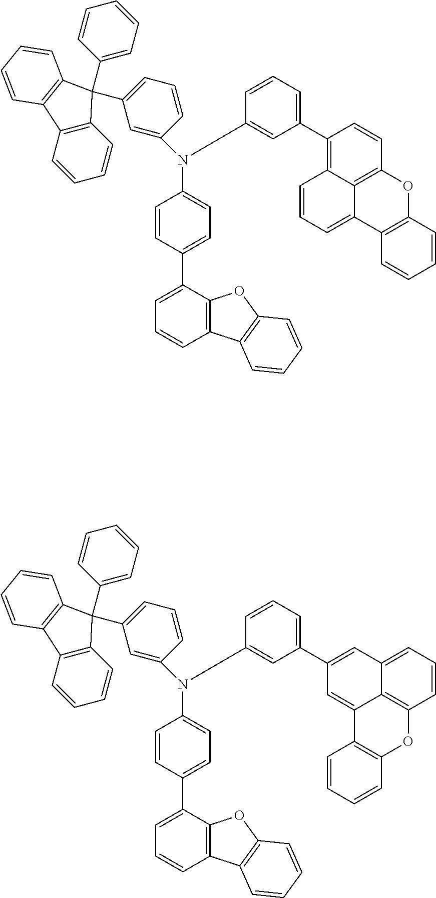 [SMILES: c1ccc(C2(c3cccc(N(c4ccc(-c5cccc6c5oc5ccccc56)cc4)c4cccc(-c5ccc6c7c(cccc57)-c5ccccc5O6)c4)c3)c3ccccc3-c3ccccc32)cc1 ; c1ccc(C2(c3cccc(N(c4ccc(-c5cccc6c5oc5ccccc56)cc4)c4cccc(-c5cc6c7c(cccc7c5)Oc5ccccc5-6)c4)c3)c3ccccc3-c3ccccc32)cc1]

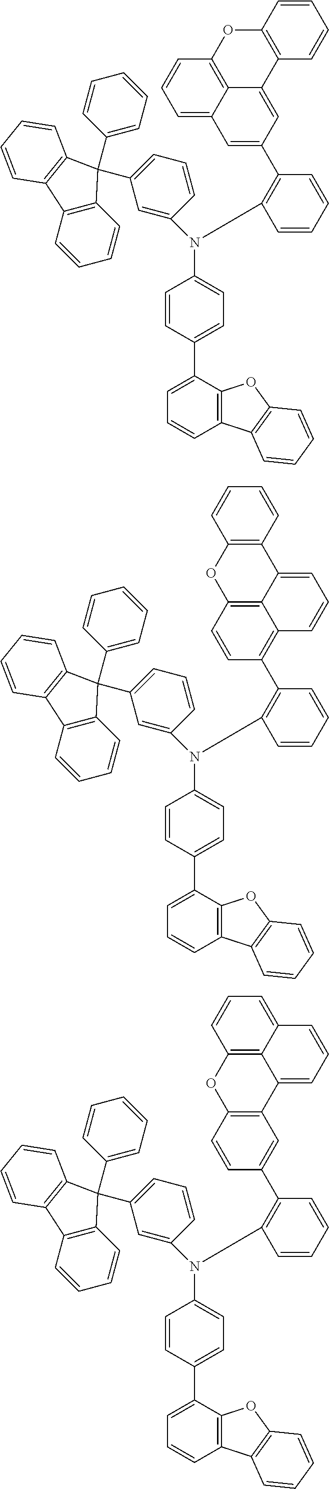

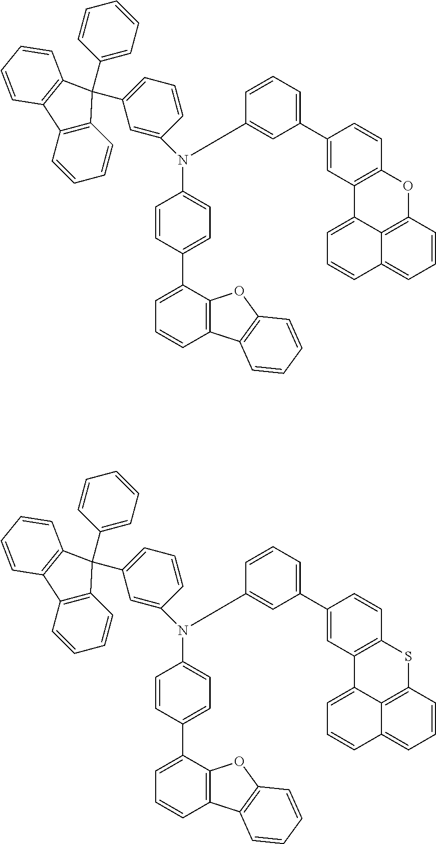 [SMILES: c1ccc(C2(c3cccc(N(c4ccc(-c5cccc6c5oc5ccccc56)cc4)c4cccc(-c5ccc6c(c5)-c5cccc7cccc(c57)O6)c4)c3)c3ccccc3-c3ccccc32)cc1 ; c1ccc(C2(c3cccc(N(c4ccc(-c5cccc6c5oc5ccccc56)cc4)c4cccc(-c5ccc6c(c5)-c5cccc7cccc(c57)S6)c4)c3)c3ccccc3-c3ccccc32)cc1]

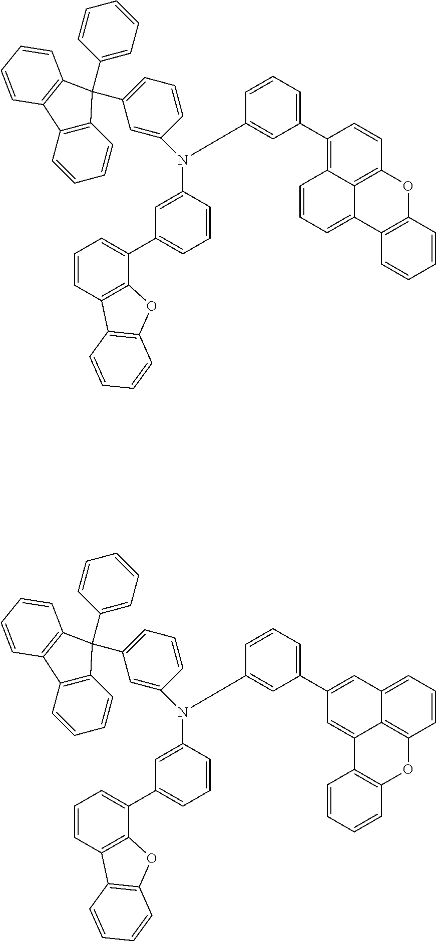 [SMILES: c1ccc(C2(c3cccc(N(c4cccc(-c5ccc6c7c(cccc57)-c5ccccc5O6)c4)c4cccc(-c5cccc6c5oc5ccccc56)c4)c3)c3ccccc3-c3ccccc32)cc1 ; c1ccc(C2(c3cccc(N(c4cccc(-c5cc6c7c(cccc7c5)Oc5ccccc5-6)c4)c4cccc(-c5cccc6c5oc5ccccc56)c4)c3)c3ccccc3-c3ccccc32)cc1]

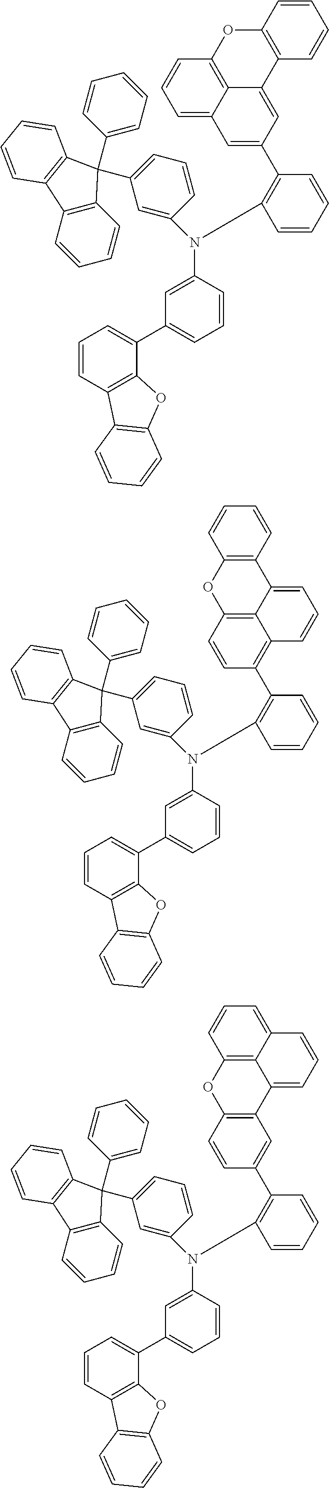 [SMILES: c1ccc(C2(c3cccc(N(c4cccc(-c5cccc6c5oc5ccccc56)c4)c4ccccc4-c4cc5c6c(cccc6c4)Oc4ccccc4-5)c3)c3ccccc3-c3ccccc32)cc1 ; c1ccc(C2(c3cccc(N(c4cccc(-c5cccc6c5oc5ccccc56)c4)c4ccccc4-c4ccc5c6c(cccc46)-c4ccccc4O5)c3)c3ccccc3-c3ccccc32)cc1 ; c1ccc(C2(c3cccc(N(c4cccc(-c5cccc6c5oc5ccccc56)c4)c4ccccc4-c4ccc5c(c4)-c4cccc6cccc(c46)O5)c3)c3ccccc3-c3ccccc32)cc1]

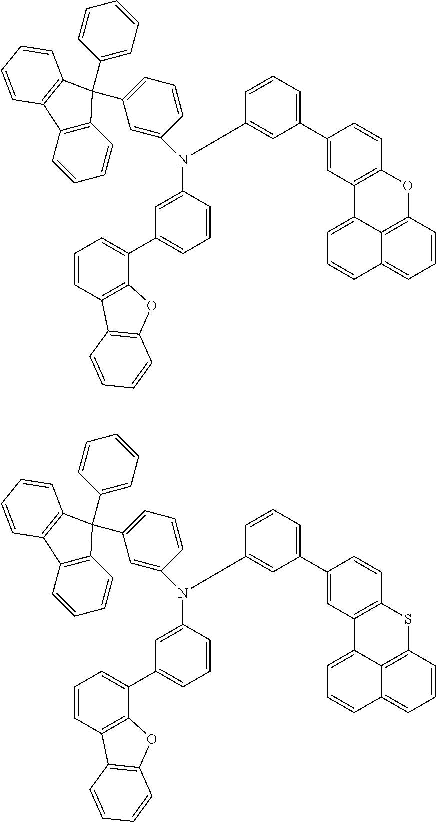 [SMILES: c1ccc(C2(c3cccc(N(c4cccc(-c5ccc6c(c5)-c5cccc7cccc(c57)O6)c4)c4cccc(-c5cccc6c5oc5ccccc56)c4)c3)c3ccccc3-c3ccccc32)cc1 ; c1ccc(C2(c3cccc(N(c4cccc(-c5ccc6c(c5)-c5cccc7cccc(c57)S6)c4)c4cccc(-c5cccc6c5oc5ccccc56)c4)c3)c3ccccc3-c3ccccc32)cc1]

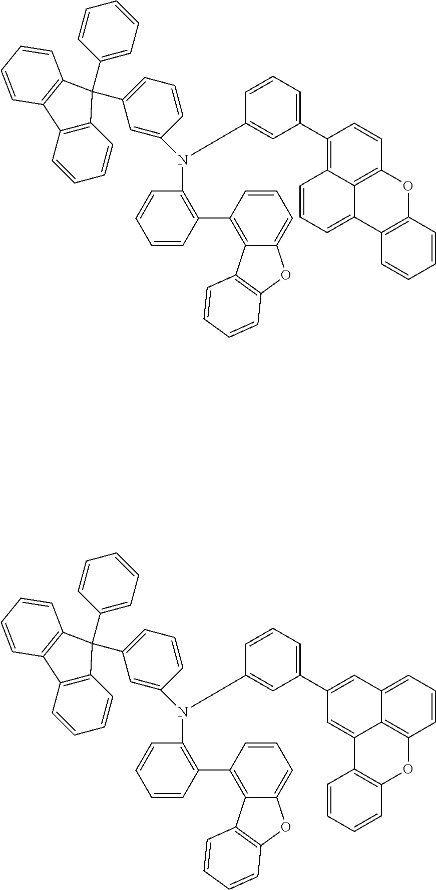 [SMILES: c1ccc(C2(c3cccc(N(c4cccc(-c5ccc6c7c(cccc57)-c5ccccc5O6)c4)c4ccccc4-c4cccc5oc6ccccc6c45)c3)c3ccccc3-c3ccccc32)cc1 ; c1ccc(C2(c3cccc(N(c4cccc(-c5cc6c7c(cccc7c5)Oc5ccccc5-6)c4)c4ccccc4-c4cccc5oc6ccccc6c45)c3)c3ccccc3-c3ccccc32)cc1]

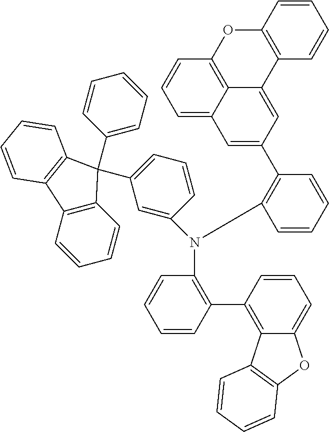 [SMILES: c1ccc(C2(c3cccc(N(c4ccccc4-c4cc5c6c(cccc6c4)Oc4ccccc4-5)c4ccccc4-c4cccc5oc6ccccc6c45)c3)c3ccccc3-c3ccccc32)cc1]

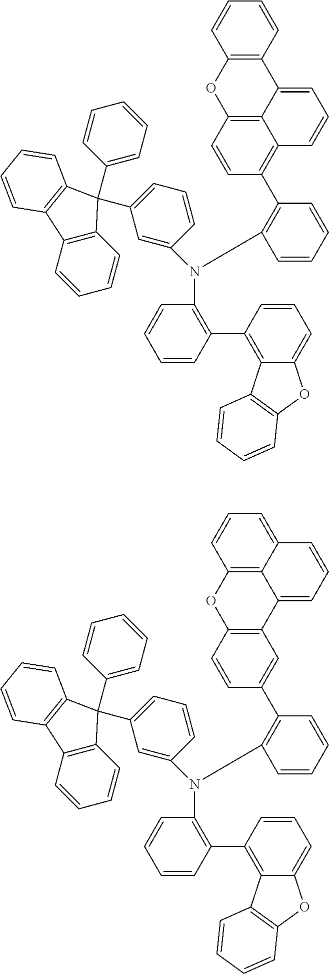 [SMILES: c1ccc(C2(c3cccc(N(c4ccccc4-c4ccc5c6c(cccc46)-c4ccccc4O5)c4ccccc4-c4cccc5oc6ccccc6c45)c3)c3ccccc3-c3ccccc32)cc1 ; c1ccc(C2(c3cccc(N(c4ccccc4-c4ccc5c(c4)-c4cccc6cccc(c46)O5)c4ccccc4-c4cccc5oc6ccccc6c45)c3)c3ccccc3-c3ccccc32)cc1]

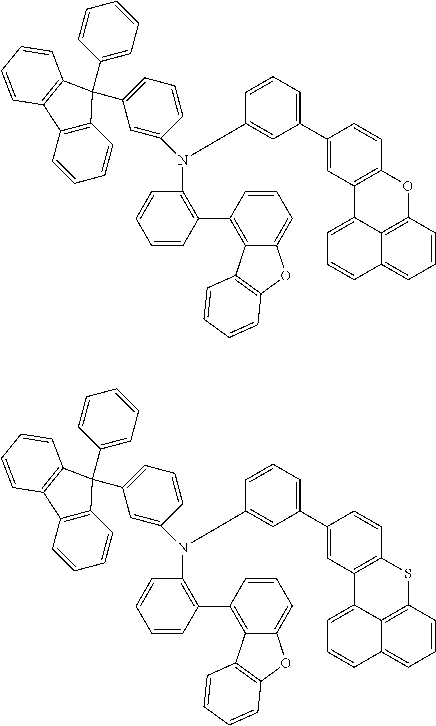 [SMILES: c1ccc(C2(c3cccc(N(c4cccc(-c5ccc6c(c5)-c5cccc7cccc(c57)O6)c4)c4ccccc4-c4cccc5oc6ccccc6c45)c3)c3ccccc3-c3ccccc32)cc1 ; c1ccc(C2(c3cccc(N(c4cccc(-c5ccc6c(c5)-c5cccc7cccc(c57)S6)c4)c4ccccc4-c4cccc5oc6ccccc6c45)c3)c3ccccc3-c3ccccc32)cc1]

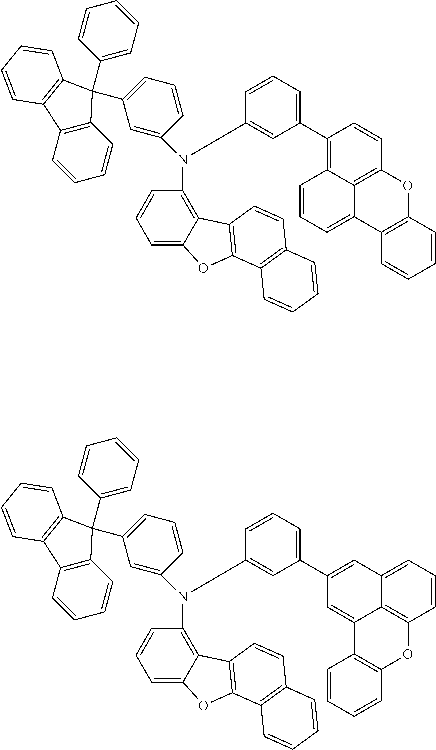 [SMILES: c1ccc(C2(c3cccc(N(c4cccc(-c5ccc6c7c(cccc57)-c5ccccc5O6)c4)c4cccc5oc6c7ccccc7ccc6c45)c3)c3ccccc3-c3ccccc32)cc1 ; c1ccc(C2(c3cccc(N(c4cccc(-c5cc6c7c(cccc7c5)Oc5ccccc5-6)c4)c4cccc5oc6c7ccccc7ccc6c45)c3)c3ccccc3-c3ccccc32)cc1]

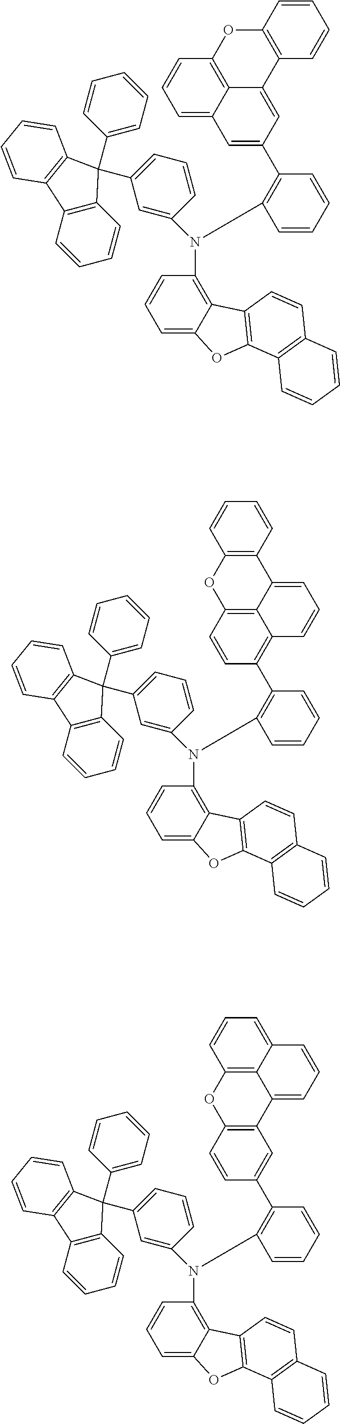 [SMILES: c1ccc(C2(c3cccc(N(c4ccccc4-c4cc5c6c(cccc6c4)Oc4ccccc4-5)c4cccc5oc6c7ccccc7ccc6c45)c3)c3ccccc3-c3ccccc32)cc1 ; c1ccc(C2(c3cccc(N(c4ccccc4-c4ccc5c6c(cccc46)-c4ccccc4O5)c4cccc5oc6c7ccccc7ccc6c45)c3)c3ccccc3-c3ccccc32)cc1 ; c1ccc(C2(c3cccc(N(c4ccccc4-c4ccc5c(c4)-c4cccc6cccc(c46)O5)c4cccc5oc6c7ccccc7ccc6c45)c3)c3ccccc3-c3ccccc32)cc1]

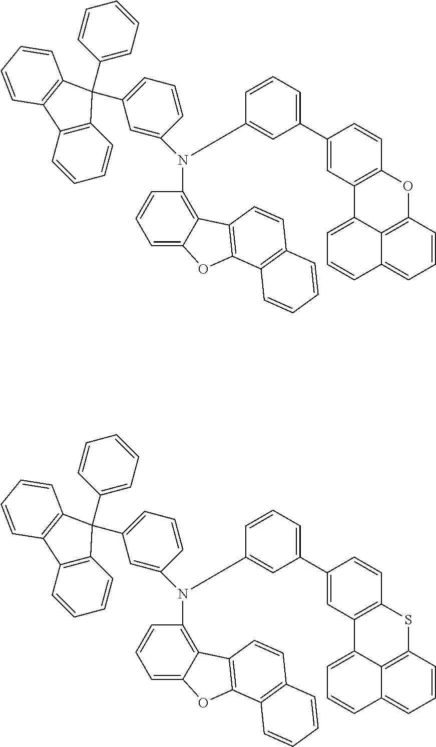 [SMILES: c1ccc(C2(c3cccc(N(c4cccc(-c5ccc6c(c5)-c5cccc7cccc(c57)O6)c4)c4cccc5oc6c7ccccc7ccc6c45)c3)c3ccccc3-c3ccccc32)cc1 ; c1ccc(C2(c3cccc(N(c4cccc(-c5ccc6c(c5)-c5cccc7cccc(c57)S6)c4)c4cccc5oc6c7ccccc7ccc6c45)c3)c3ccccc3-c3ccccc32)cc1]

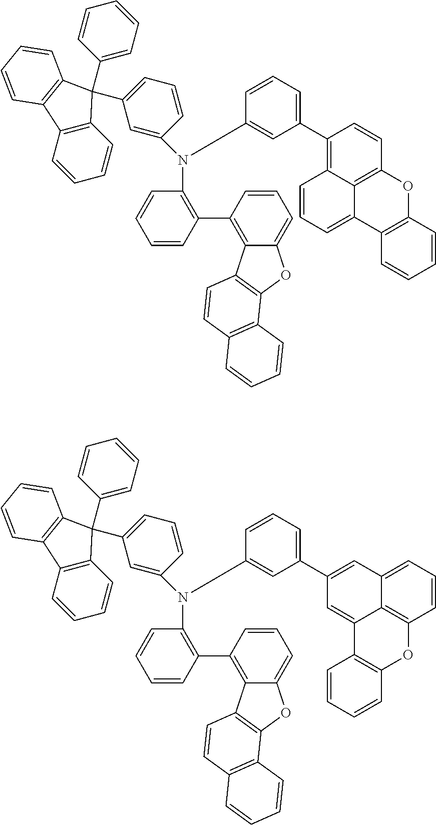 [SMILES: c1ccc(C2(c3cccc(N(c4cccc(-c5ccc6c7c(cccc57)-c5ccccc5O6)c4)c4ccccc4-c4cccc5oc6c7ccccc7ccc6c45)c3)c3ccccc3-c3ccccc32)cc1 ; c1ccc(C2(c3cccc(N(c4cccc(-c5cc6c7c(cccc7c5)Oc5ccccc5-6)c4)c4ccccc4-c4cccc5oc6c7ccccc7ccc6c45)c3)c3ccccc3-c3ccccc32)cc1]

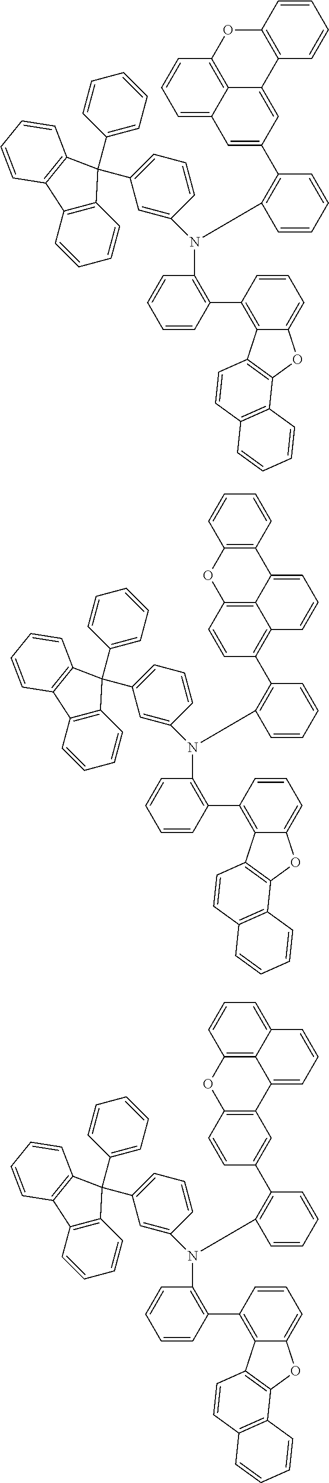

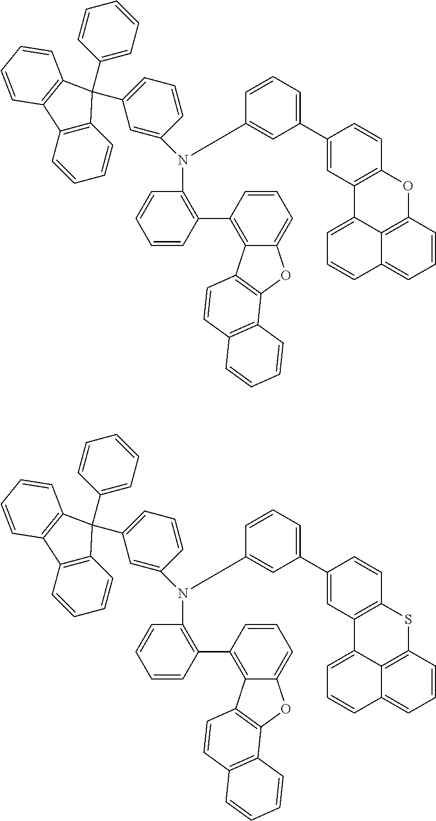 [SMILES: c1ccc(C2(c3cccc(N(c4cccc(-c5ccc6c(c5)-c5cccc7cccc(c57)O6)c4)c4ccccc4-c4cccc5oc6c7ccccc7ccc6c45)c3)c3ccccc3-c3ccccc32)cc1 ; c1ccc(C2(c3cccc(N(c4cccc(-c5ccc6c(c5)-c5cccc7cccc(c57)S6)c4)c4ccccc4-c4cccc5oc6c7ccccc7ccc6c45)c3)c3ccccc3-c3ccccc32)cc1]

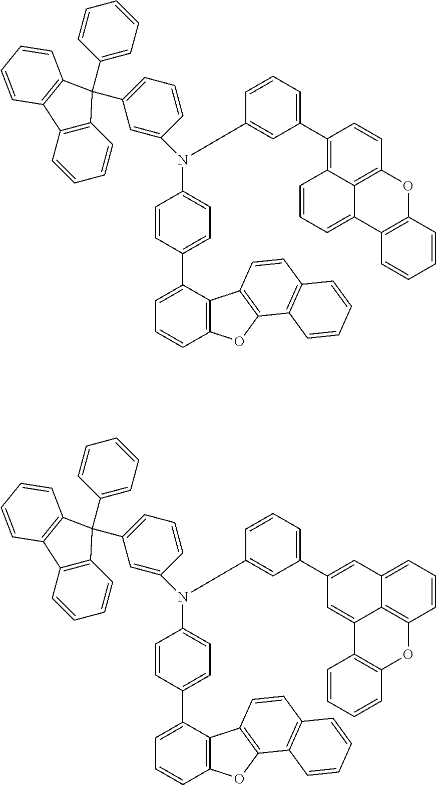 [SMILES: c1ccc(C2(c3cccc(N(c4ccc(-c5cccc6oc7c8ccccc8ccc7c56)cc4)c4cccc(-c5ccc6c7c(cccc57)-c5ccccc5O6)c4)c3)c3ccccc3-c3ccccc32)cc1 ; c1ccc(C2(c3cccc(N(c4ccc(-c5cccc6oc7c8ccccc8ccc7c56)cc4)c4cccc(-c5cc6c7c(cccc7c5)Oc5ccccc5-6)c4)c3)c3ccccc3-c3ccccc32)cc1]

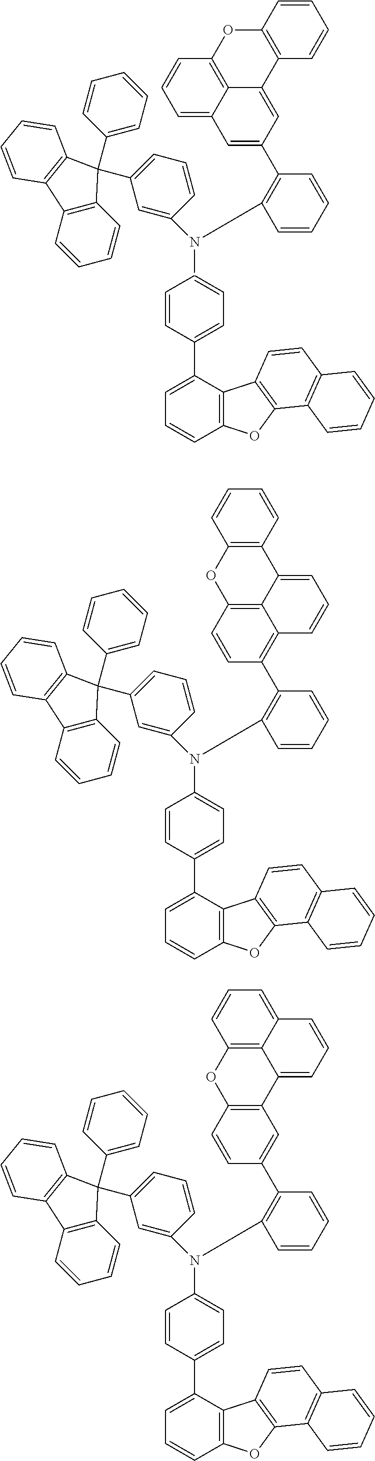

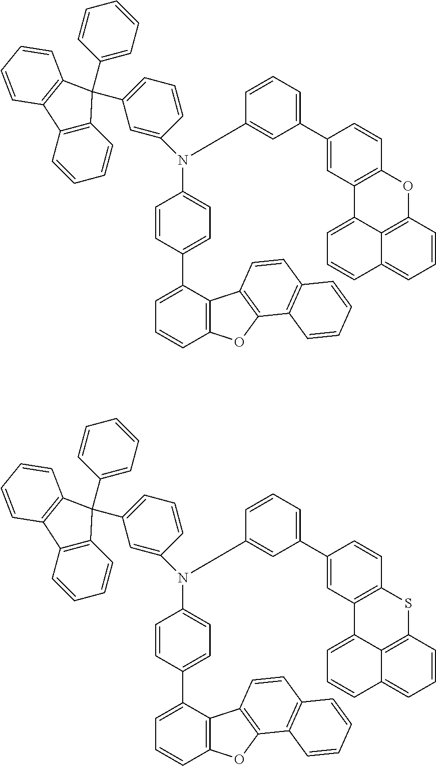 [SMILES: c1ccc(C2(c3cccc(N(c4ccc(-c5cccc6oc7c8ccccc8ccc7c56)cc4)c4cccc(-c5ccc6c(c5)-c5cccc7cccc(c57)O6)c4)c3)c3ccccc3-c3ccccc32)cc1 ; c1ccc(C2(c3cccc(N(c4ccc(-c5cccc6oc7c8ccccc8ccc7c56)cc4)c4cccc(-c5ccc6c(c5)-c5cccc7cccc(c57)S6)c4)c3)c3ccccc3-c3ccccc32)cc1]

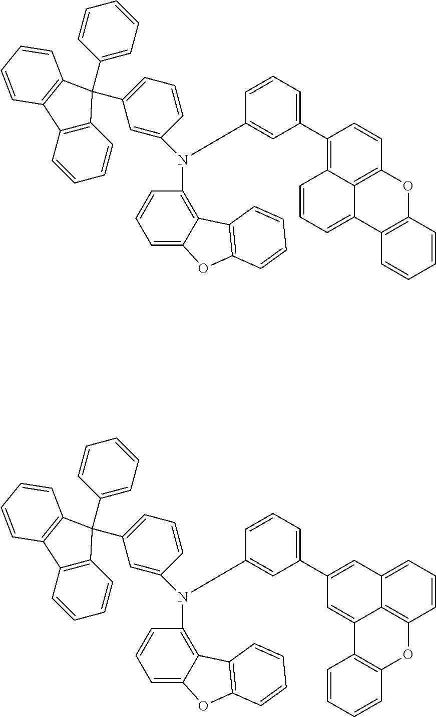 [SMILES: c1ccc(C2(c3cccc(N(c4cccc(-c5ccc6c7c(cccc57)-c5ccccc5O6)c4)c4cccc5oc6ccccc6c45)c3)c3ccccc3-c3ccccc32)cc1 ; c1ccc(C2(c3cccc(N(c4cccc(-c5cc6c7c(cccc7c5)Oc5ccccc5-6)c4)c4cccc5oc6ccccc6c45)c3)c3ccccc3-c3ccccc32)cc1]

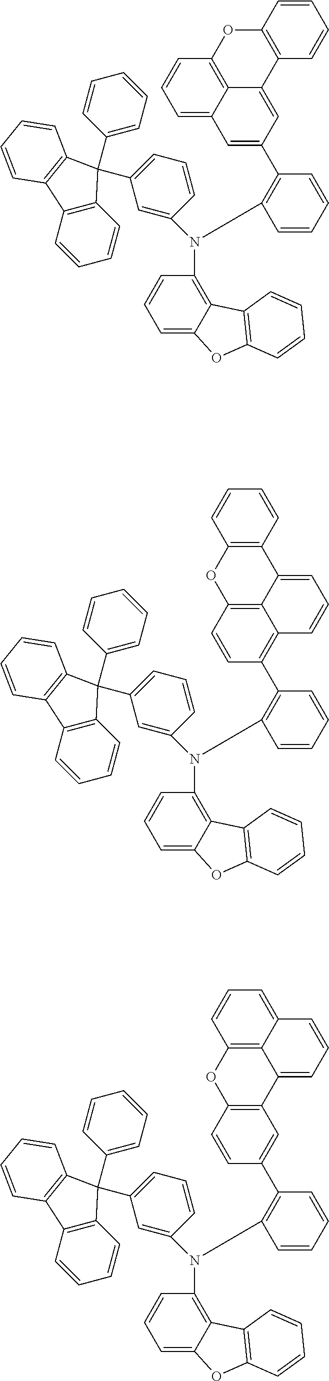 [SMILES: c1ccc(C2(c3cccc(N(c4ccccc4-c4cc5c6c(cccc6c4)Oc4ccccc4-5)c4cccc5oc6ccccc6c45)c3)c3ccccc3-c3ccccc32)cc1 ; c1ccc(C2(c3cccc(N(c4ccccc4-c4ccc5c6c(cccc46)-c4ccccc4O5)c4cccc5oc6ccccc6c45)c3)c3ccccc3-c3ccccc32)cc1 ; c1ccc(C2(c3cccc(N(c4ccccc4-c4ccc5c(c4)-c4cccc6cccc(c46)O5)c4cccc5oc6ccccc6c45)c3)c3ccccc3-c3ccccc32)cc1]

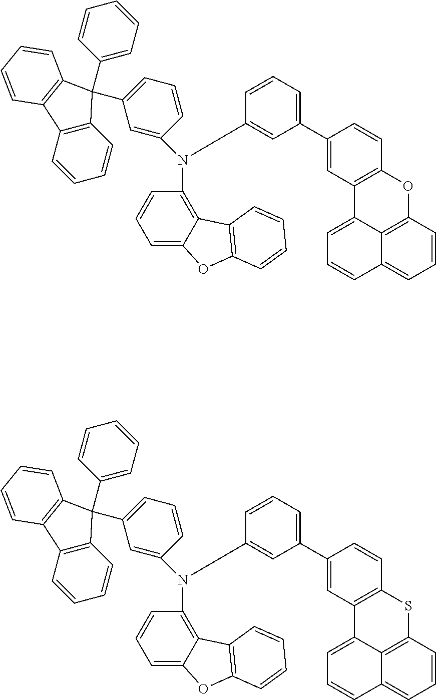 [SMILES: c1ccc(C2(c3cccc(N(c4cccc(-c5ccc6c(c5)-c5cccc7cccc(c57)O6)c4)c4cccc5oc6ccccc6c45)c3)c3ccccc3-c3ccccc32)cc1 ; c1ccc(C2(c3cccc(N(c4cccc(-c5ccc6c(c5)-c5cccc7cccc(c57)S6)c4)c4cccc5oc6ccccc6c45)c3)c3ccccc3-c3ccccc32)cc1]

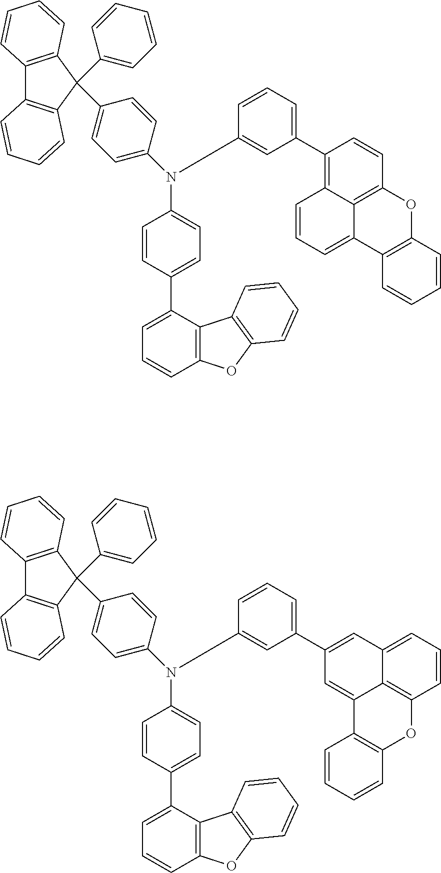 [SMILES: c1ccc(C2(c3ccc(N(c4ccc(-c5cccc6oc7ccccc7c56)cc4)c4cccc(-c5ccc6c7c(cccc57)-c5ccccc5O6)c4)cc3)c3ccccc3-c3ccccc32)cc1 ; c1ccc(C2(c3ccc(N(c4ccc(-c5cccc6oc7ccccc7c56)cc4)c4cccc(-c5cc6c7c(cccc7c5)Oc5ccccc5-6)c4)cc3)c3ccccc3-c3ccccc32)cc1]

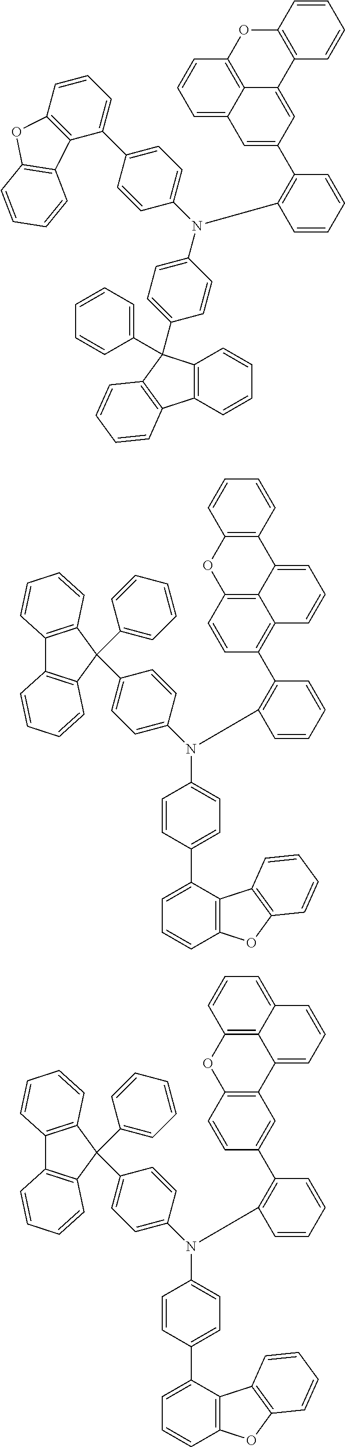

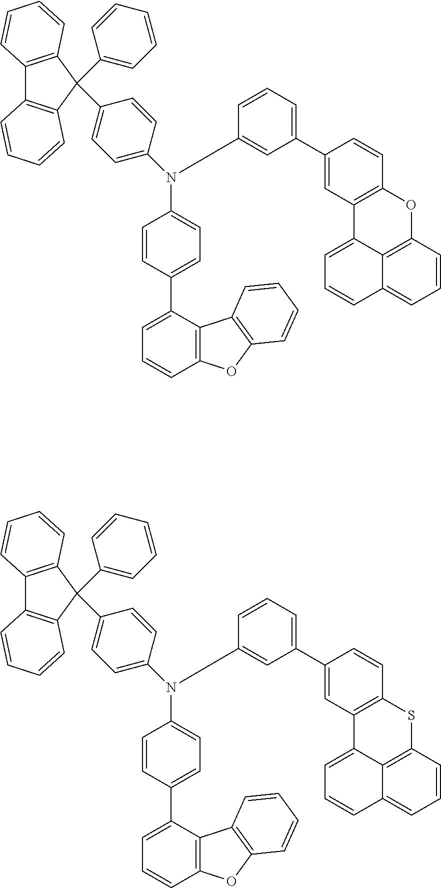 [SMILES: c1ccc(C2(c3ccc(N(c4ccc(-c5cccc6oc7ccccc7c56)cc4)c4cccc(-c5ccc6c(c5)-c5cccc7cccc(c57)O6)c4)cc3)c3ccccc3-c3ccccc32)cc1 ; c1ccc(C2(c3ccc(N(c4ccc(-c5cccc6oc7ccccc7c56)cc4)c4cccc(-c5ccc6c(c5)-c5cccc7cccc(c57)S6)c4)cc3)c3ccccc3-c3ccccc32)cc1]

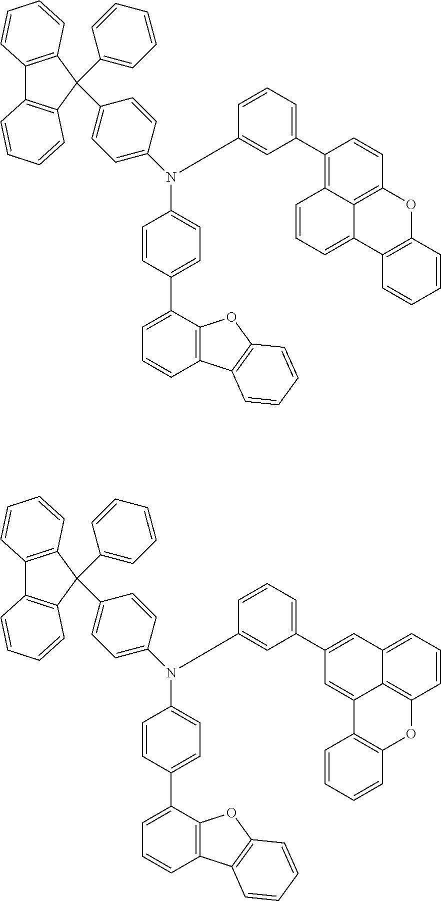 [SMILES: c1ccc(C2(c3ccc(N(c4ccc(-c5cccc6c5oc5ccccc56)cc4)c4cccc(-c5ccc6c7c(cccc57)-c5ccccc5O6)c4)cc3)c3ccccc3-c3ccccc32)cc1 ; c1ccc(C2(c3ccc(N(c4ccc(-c5cccc6c5oc5ccccc56)cc4)c4cccc(-c5cc6c7c(cccc7c5)Oc5ccccc5-6)c4)cc3)c3ccccc3-c3ccccc32)cc1]

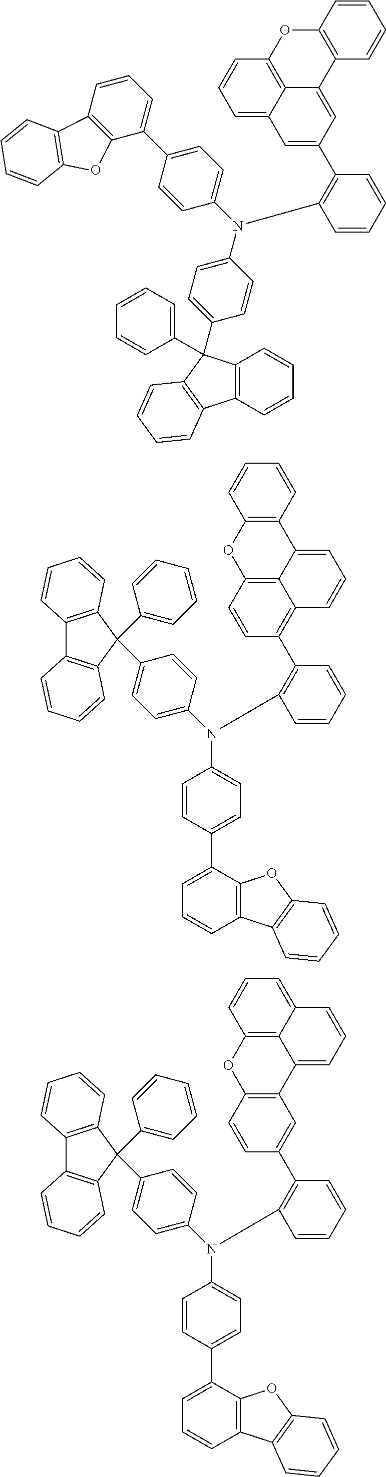

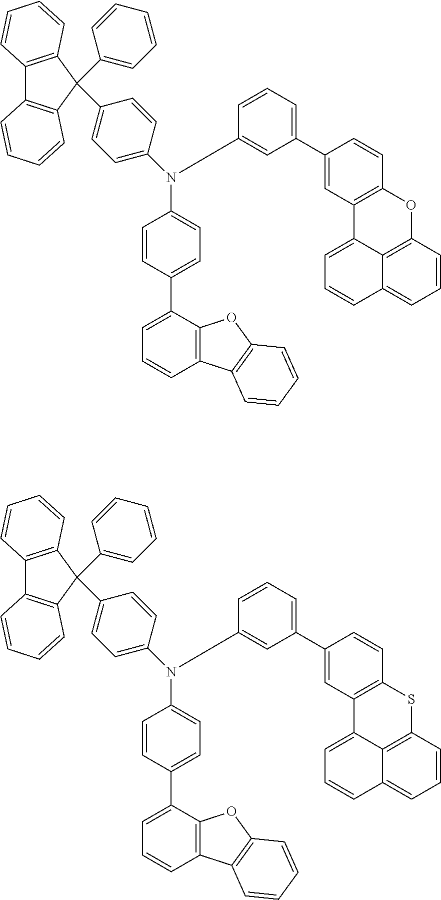 [SMILES: c1ccc(C2(c3ccc(N(c4ccc(-c5cccc6c5oc5ccccc56)cc4)c4cccc(-c5ccc6c(c5)-c5cccc7cccc(c57)O6)c4)cc3)c3ccccc3-c3ccccc32)cc1 ; c1ccc(C2(c3ccc(N(c4ccc(-c5cccc6c5oc5ccccc56)cc4)c4cccc(-c5ccc6c(c5)-c5cccc7cccc(c57)S6)c4)cc3)c3ccccc3-c3ccccc32)cc1]

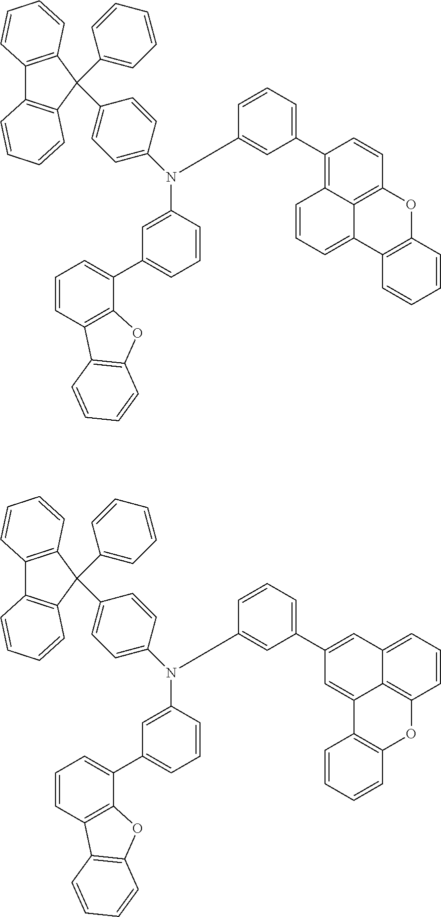 [SMILES: c1ccc(C2(c3ccc(N(c4cccc(-c5ccc6c7c(cccc57)-c5ccccc5O6)c4)c4cccc(-c5cccc6c5oc5ccccc56)c4)cc3)c3ccccc3-c3ccccc32)cc1 ; c1ccc(C2(c3ccc(N(c4cccc(-c5cc6c7c(cccc7c5)Oc5ccccc5-6)c4)c4cccc(-c5cccc6c5oc5ccccc56)c4)cc3)c3ccccc3-c3ccccc32)cc1]

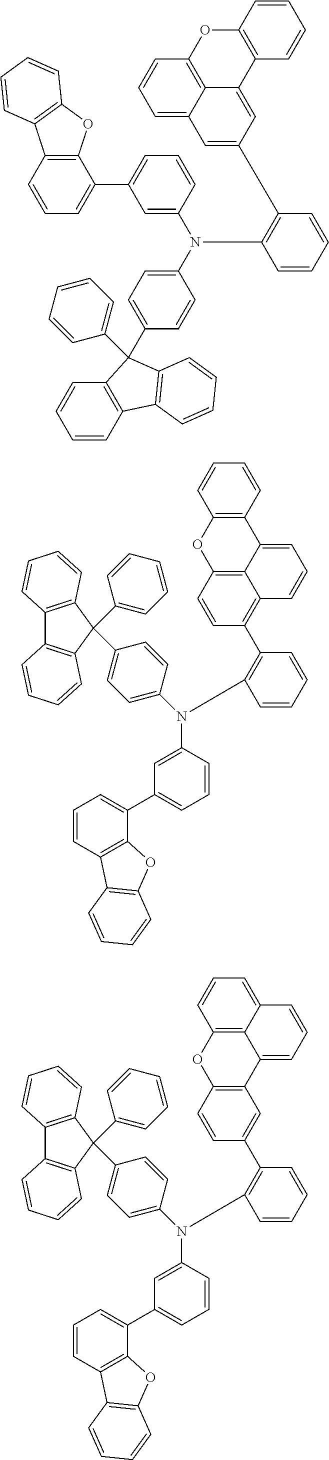 [SMILES: c1ccc(C2(c3ccc(N(c4cccc(-c5cccc6c5oc5ccccc56)c4)c4ccccc4-c4cc5c6c(cccc6c4)Oc4ccccc4-5)cc3)c3ccccc3-c3ccccc32)cc1 ; c1ccc(C2(c3ccc(N(c4cccc(-c5cccc6c5oc5ccccc56)c4)c4ccccc4-c4ccc5c6c(cccc46)-c4ccccc4O5)cc3)c3ccccc3-c3ccccc32)cc1 ; c1ccc(C2(c3ccc(N(c4cccc(-c5cccc6c5oc5ccccc56)c4)c4ccccc4-c4ccc5c(c4)-c4cccc6cccc(c46)O5)cc3)c3ccccc3-c3ccccc32)cc1]

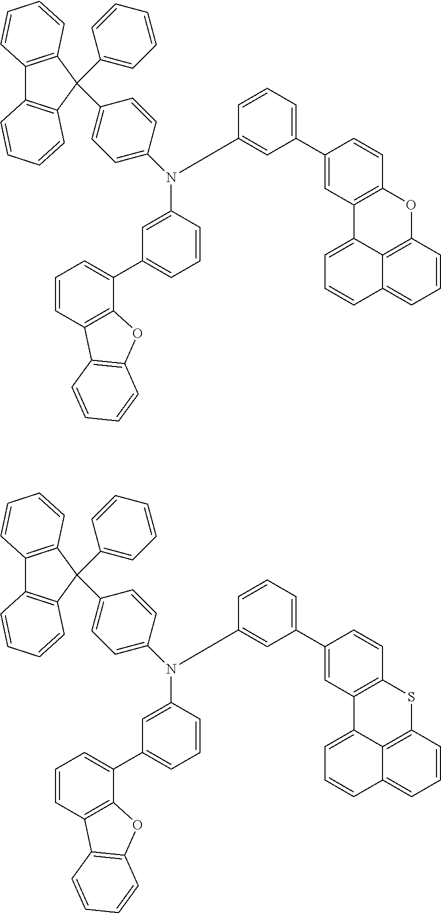 [SMILES: c1ccc(C2(c3ccc(N(c4cccc(-c5ccc6c(c5)-c5cccc7cccc(c57)O6)c4)c4cccc(-c5cccc6c5oc5ccccc56)c4)cc3)c3ccccc3-c3ccccc32)cc1 ; c1ccc(C2(c3ccc(N(c4cccc(-c5ccc6c(c5)-c5cccc7cccc(c57)S6)c4)c4cccc(-c5cccc6c5oc5ccccc56)c4)cc3)c3ccccc3-c3ccccc32)cc1]

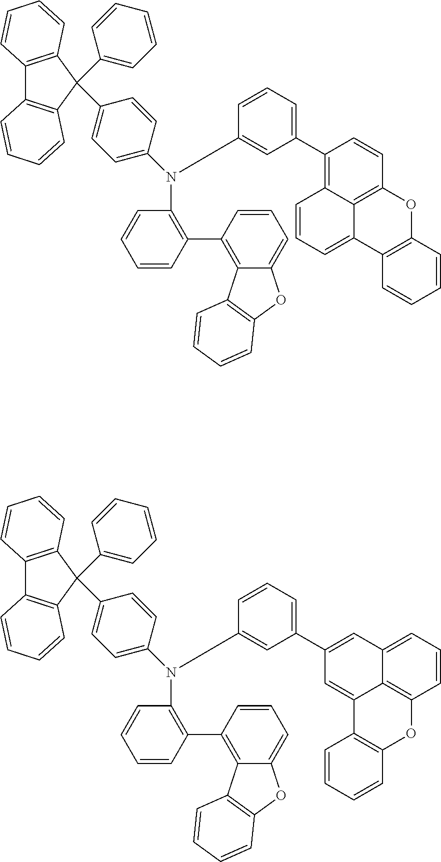 [SMILES: c1ccc(C2(c3ccc(N(c4cccc(-c5ccc6c7c(cccc57)-c5ccccc5O6)c4)c4ccccc4-c4cccc5oc6ccccc6c45)cc3)c3ccccc3-c3ccccc32)cc1 ; c1ccc(C2(c3ccc(N(c4cccc(-c5cc6c7c(cccc7c5)Oc5ccccc5-6)c4)c4ccccc4-c4cccc5oc6ccccc6c45)cc3)c3ccccc3-c3ccccc32)cc1]

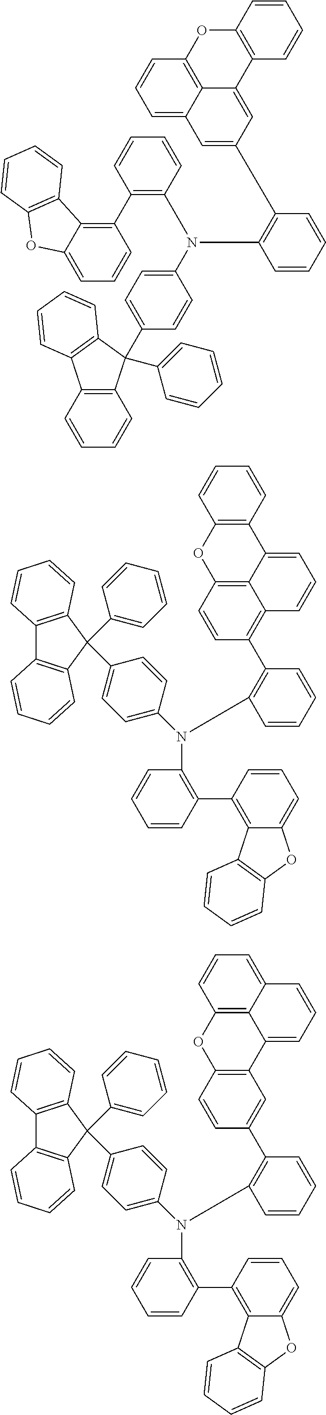

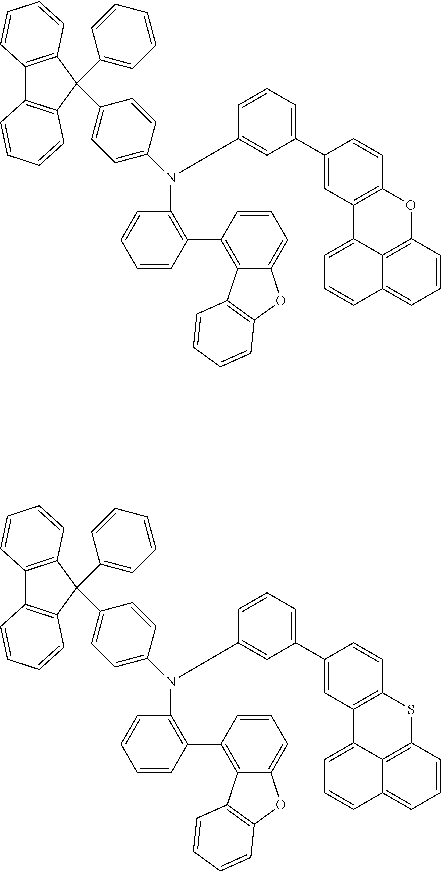 [SMILES: c1ccc(C2(c3ccc(N(c4cccc(-c5ccc6c(c5)-c5cccc7cccc(c57)O6)c4)c4ccccc4-c4cccc5oc6ccccc6c45)cc3)c3ccccc3-c3ccccc32)cc1 ; c1ccc(C2(c3ccc(N(c4cccc(-c5ccc6c(c5)-c5cccc7cccc(c57)S6)c4)c4ccccc4-c4cccc5oc6ccccc6c45)cc3)c3ccccc3-c3ccccc32)cc1]

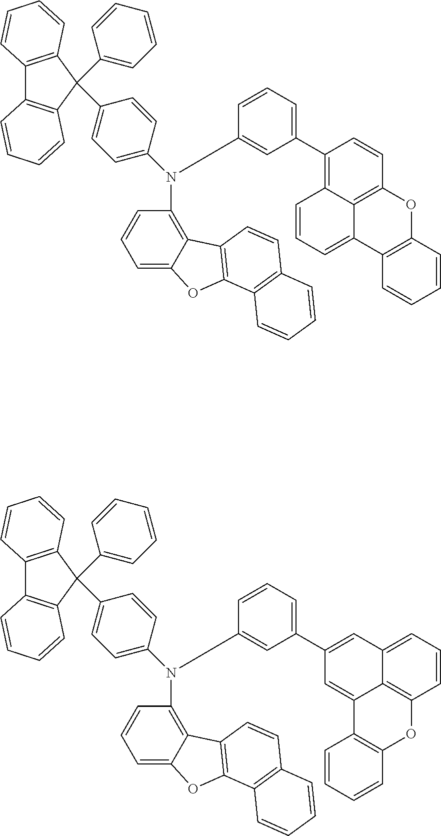 [SMILES: c1ccc(C2(c3ccc(N(c4cccc(-c5ccc6c7c(cccc57)-c5ccccc5O6)c4)c4cccc5oc6c7ccccc7ccc6c45)cc3)c3ccccc3-c3ccccc32)cc1 ; c1ccc(C2(c3ccc(N(c4cccc(-c5cc6c7c(cccc7c5)Oc5ccccc5-6)c4)c4cccc5oc6c7ccccc7ccc6c45)cc3)c3ccccc3-c3ccccc32)cc1]

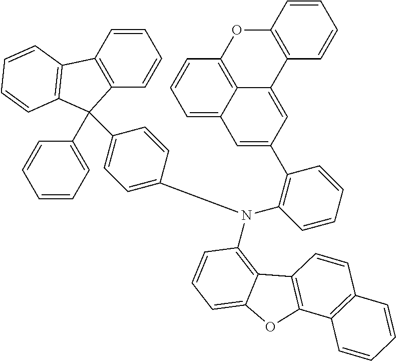 [SMILES: c1ccc(C2(c3ccc(N(c4ccccc4-c4cc5c6c(cccc6c4)Oc4ccccc4-5)c4cccc5oc6c7ccccc7ccc6c45)cc3)c3ccccc3-c3ccccc32)cc1]

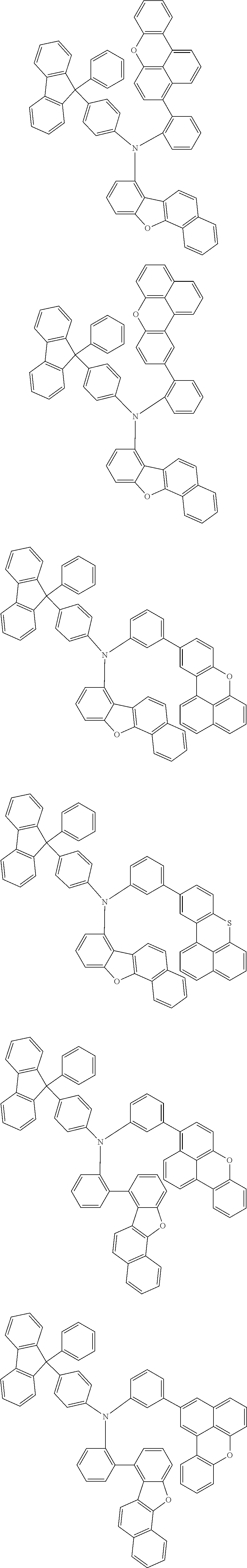

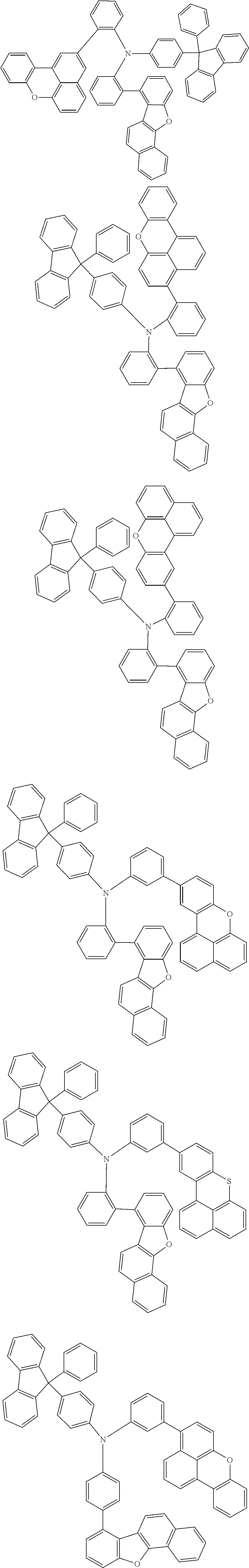

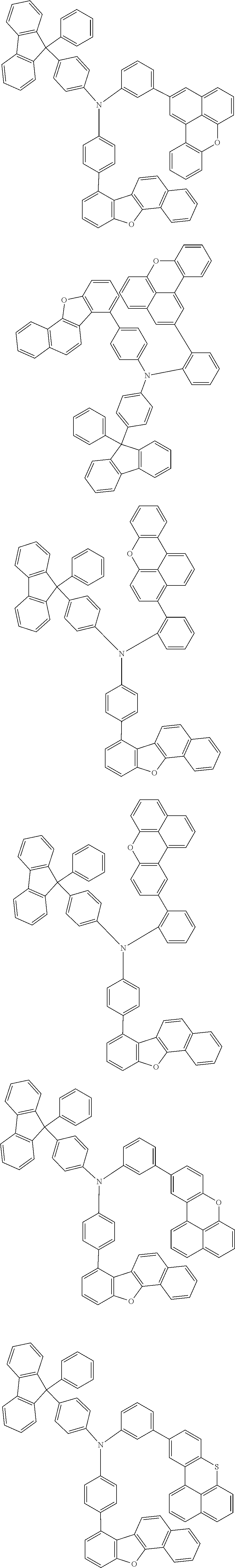

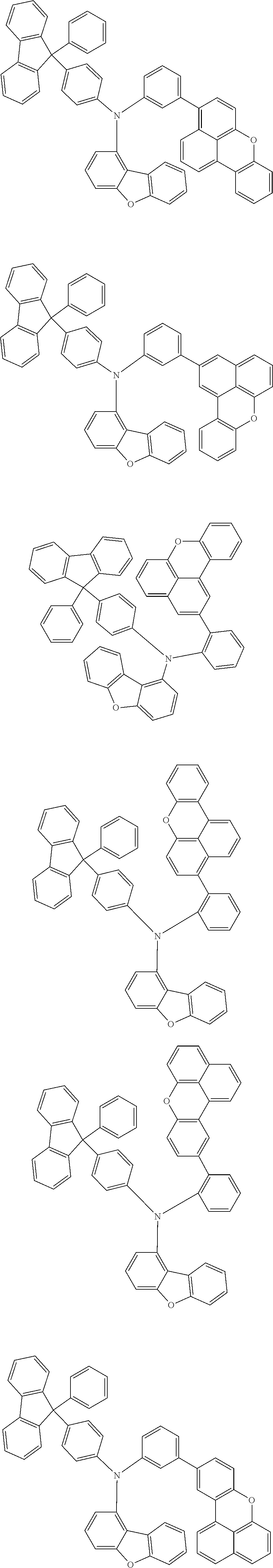

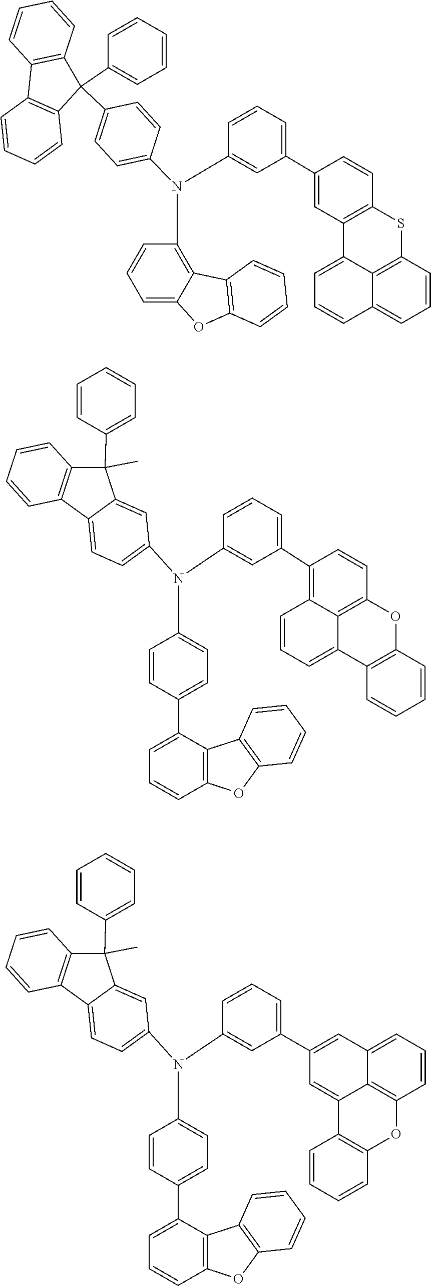 [SMILES: c1ccc(C2(c3ccc(N(c4cccc(-c5ccc6c(c5)-c5cccc7cccc(c57)S6)c4)c4cccc5oc6ccccc6c45)cc3)c3ccccc3-c3ccccc32)cc1 ; CC1(c2ccccc2)c2ccccc2-c2ccc(N(c3ccc(-c4cccc5oc6ccccc6c45)cc3)c3cccc(-c4ccc5c6c(cccc46)-c4ccccc4O5)c3)cc21 ; CC1(c2ccccc2)c2ccccc2-c2ccc(N(c3ccc(-c4cccc5oc6ccccc6c45)cc3)c3cccc(-c4cc5c6c(cccc6c4)Oc4ccccc4-5)c3)cc21]

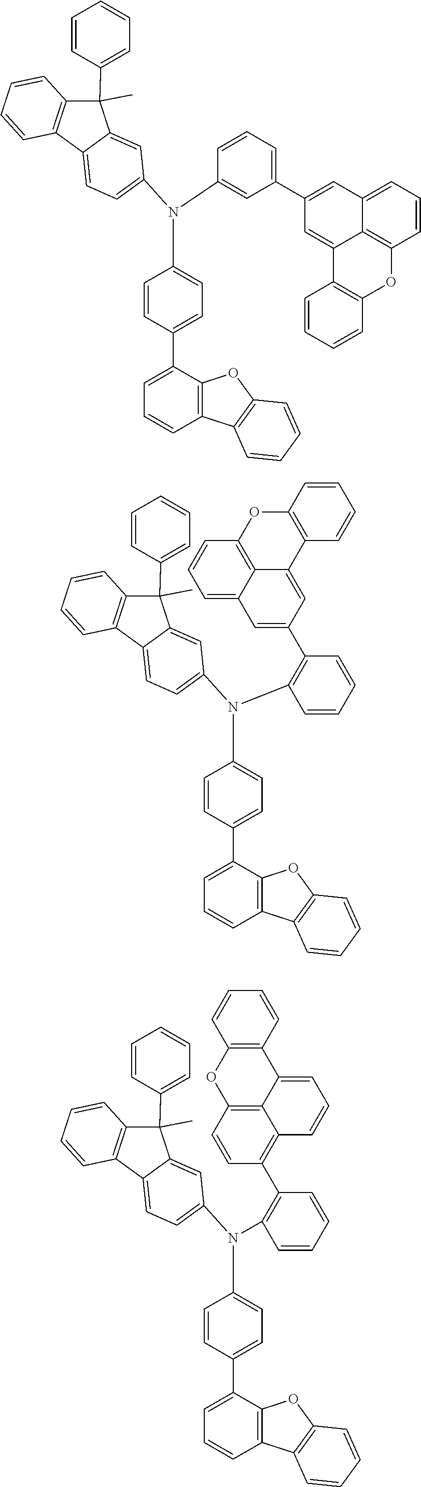

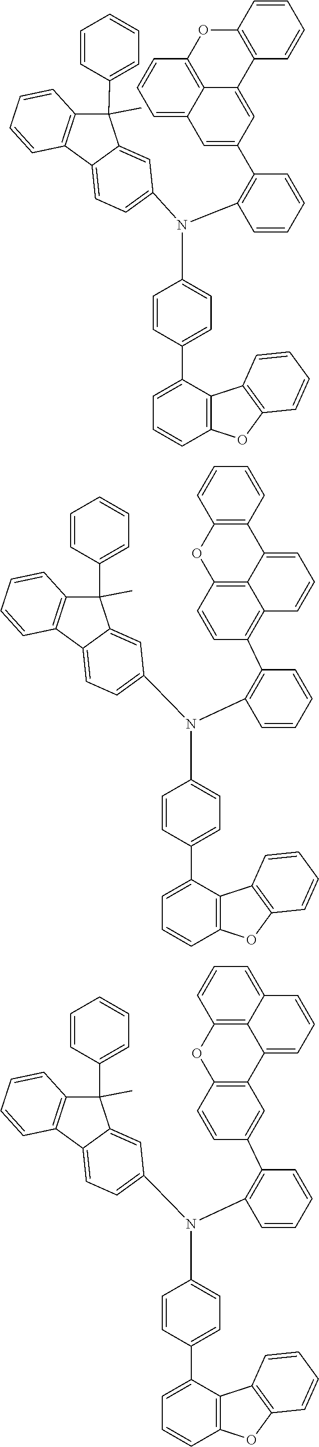

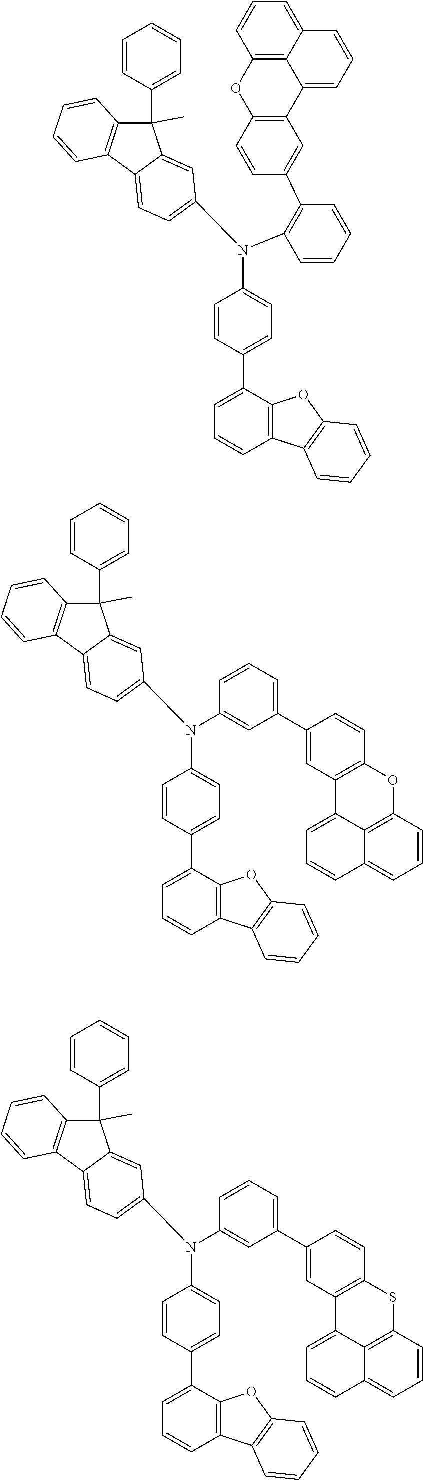 [SMILES: CC1(c2ccccc2)c2ccccc2-c2ccc(N(c3ccc(-c4cccc5c4oc4ccccc45)cc3)c3ccccc3-c3ccc4c(c3)-c3cccc5cccc(c35)O4)cc21 ; CC1(c2ccccc2)c2ccccc2-c2ccc(N(c3ccc(-c4cccc5c4oc4ccccc45)cc3)c3cccc(-c4ccc5c(c4)-c4cccc6cccc(c46)O5)c3)cc21 ; CC1(c2ccccc2)c2ccccc2-c2ccc(N(c3ccc(-c4cccc5c4oc4ccccc45)cc3)c3cccc(-c4ccc5c(c4)-c4cccc6cccc(c46)S5)c3)cc21]

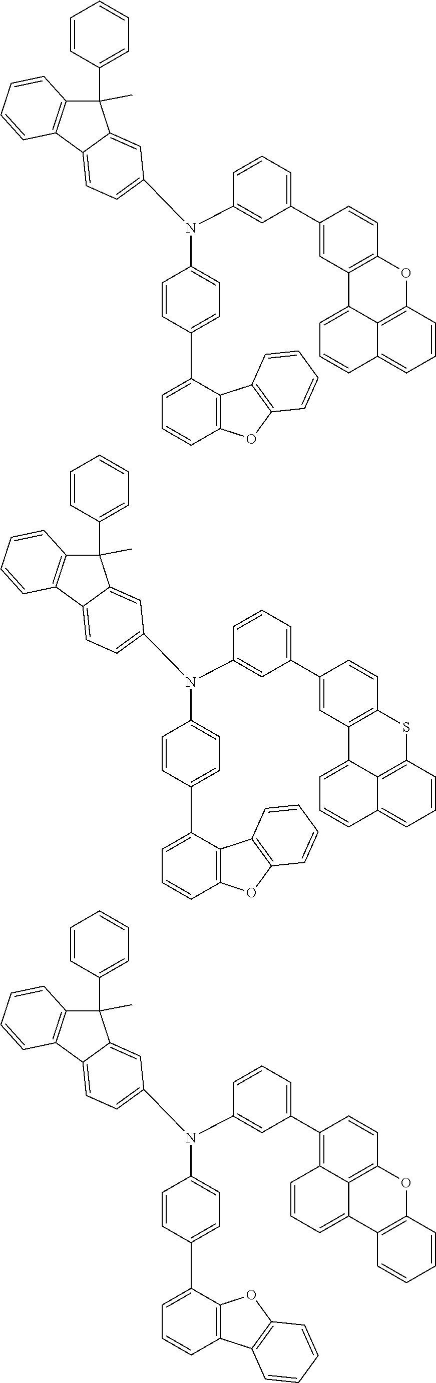 [SMILES: CC1(c2ccccc2)c2ccccc2-c2ccc(N(c3ccc(-c4cccc5oc6ccccc6c45)cc3)c3cccc(-c4ccc5c(c4)-c4cccc6cccc(c46)O5)c3)cc21 ; CC1(c2ccccc2)c2ccccc2-c2ccc(N(c3ccc(-c4cccc5oc6ccccc6c45)cc3)c3cccc(-c4ccc5c(c4)-c4cccc6cccc(c46)S5)c3)cc21 ; CC1(c2ccccc2)c2ccccc2-c2ccc(N(c3ccc(-c4cccc5c4oc4ccccc45)cc3)c3cccc(-c4ccc5c6c(cccc46)-c4ccccc4O5)c3)cc21]

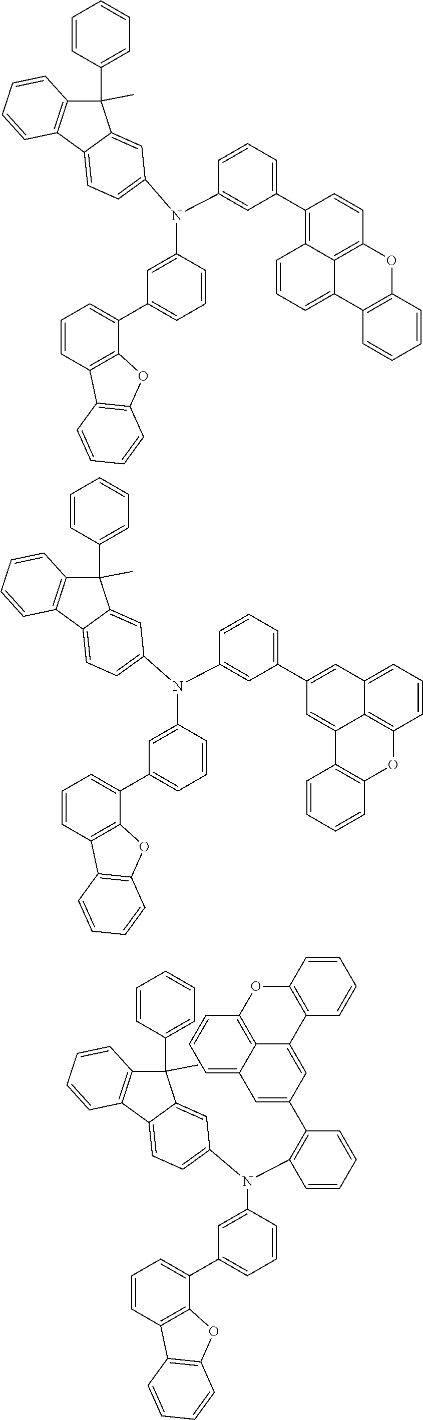

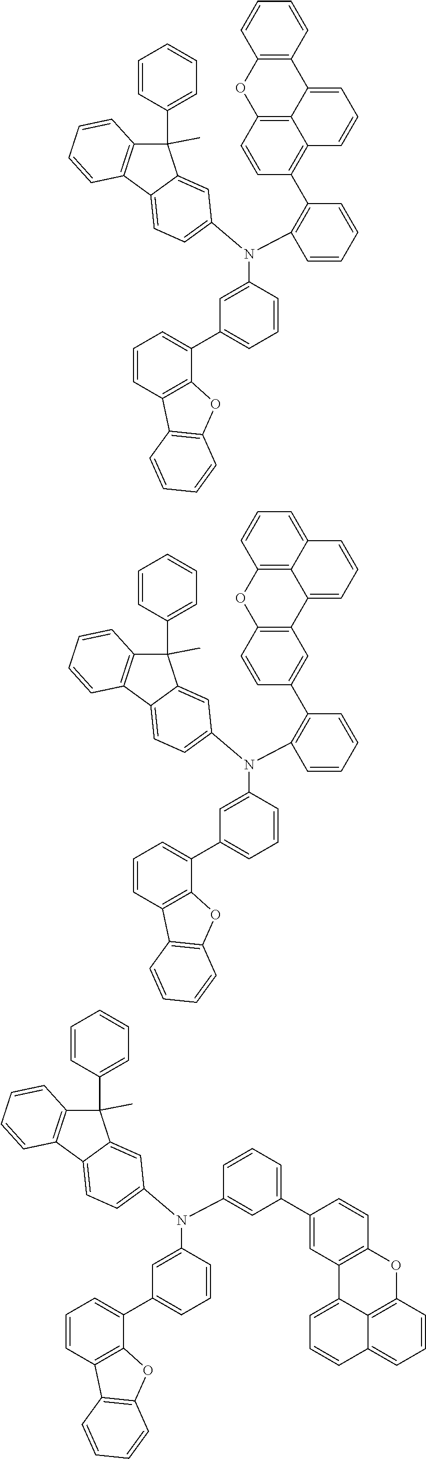

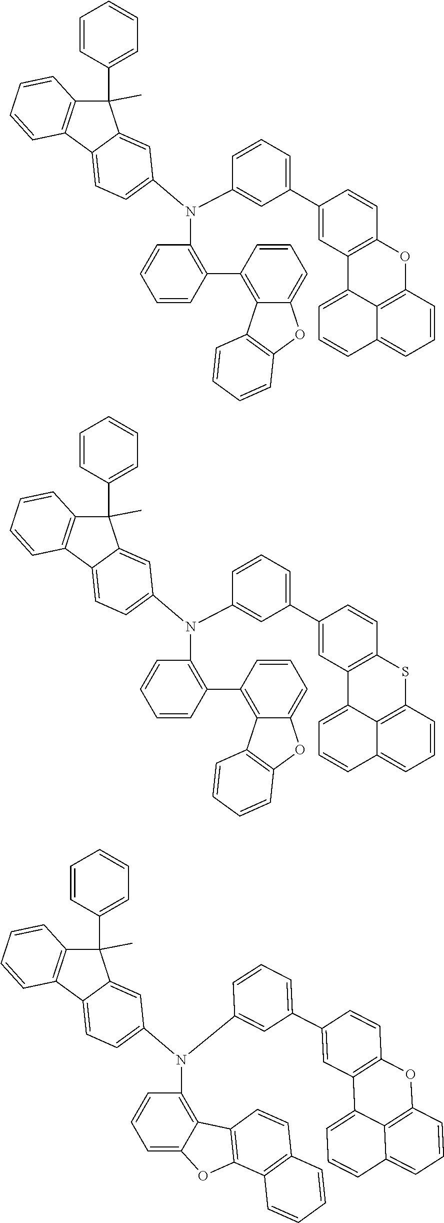 [SMILES: CC1(c2ccccc2)c2ccccc2-c2ccc(N(c3cccc(-c4ccc5c(c4)-c4cccc6cccc(c46)O5)c3)c3ccccc3-c3cccc4oc5ccccc5c34)cc21 ; CC1(c2ccccc2)c2ccccc2-c2ccc(N(c3cccc(-c4ccc5c(c4)-c4cccc6cccc(c46)S5)c3)c3ccccc3-c3cccc4oc5ccccc5c34)cc21 ; CC1(c2ccccc2)c2ccccc2-c2ccc(N(c3cccc(-c4ccc5c(c4)-c4cccc6cccc(c46)O5)c3)c3cccc4oc5c6ccccc6ccc5c34)cc21]

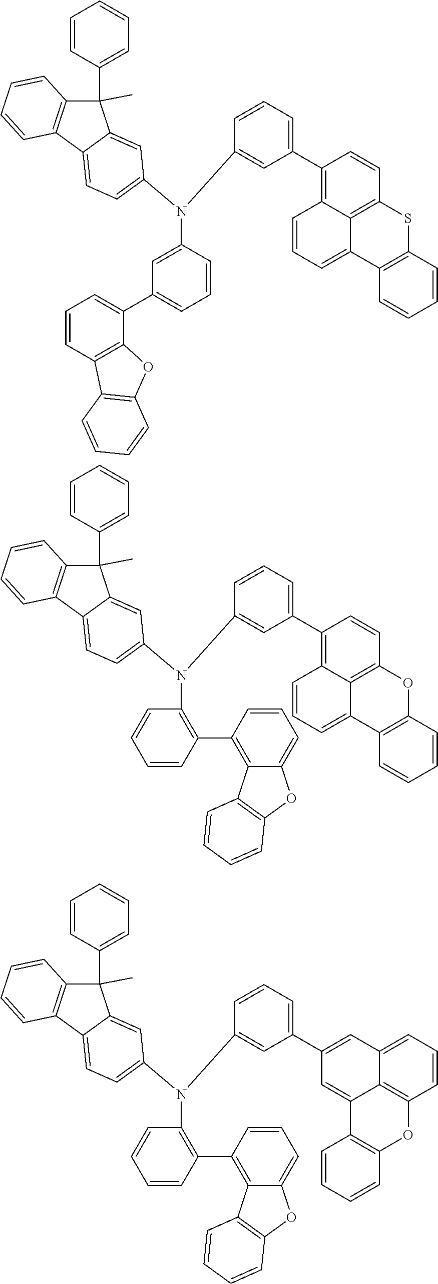

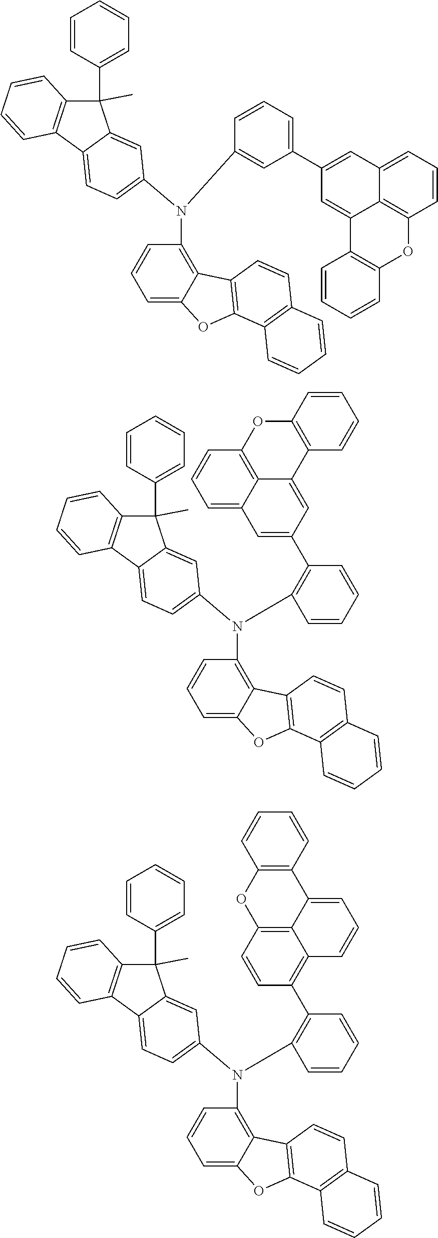

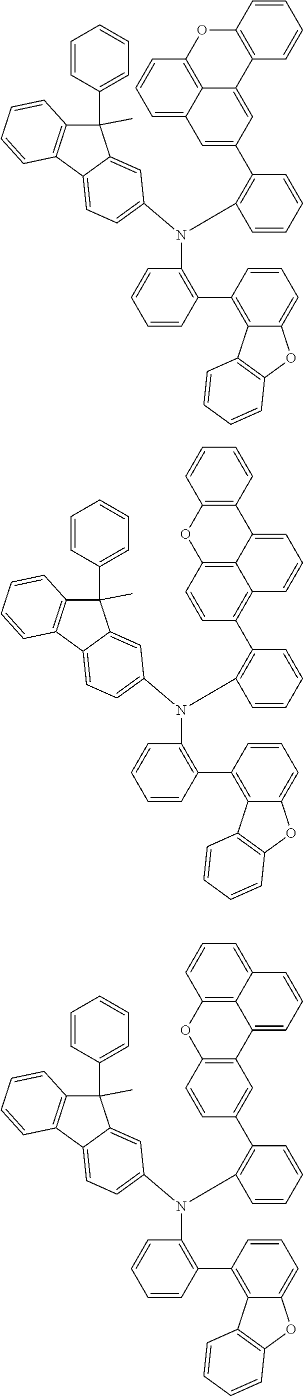

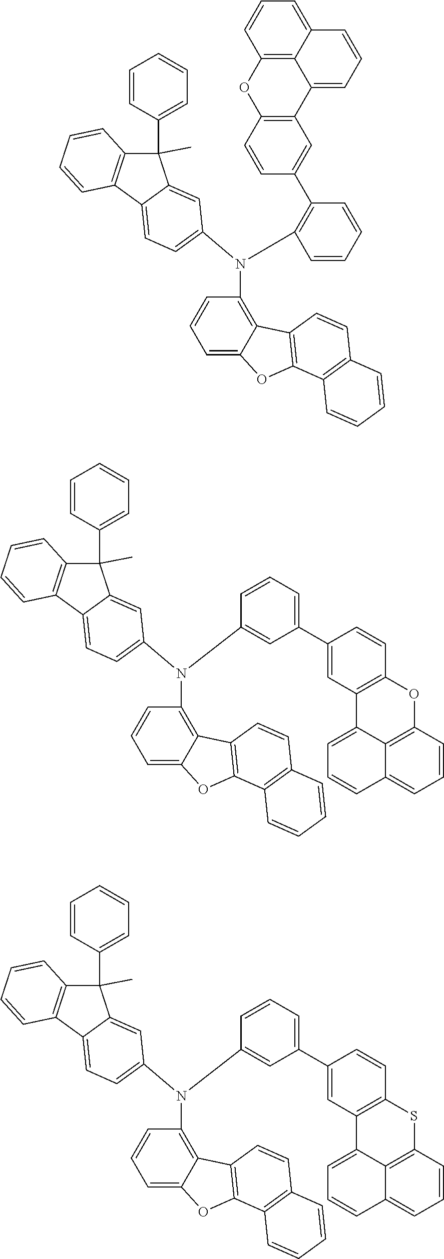 [SMILES: CC1(c2ccccc2)c2ccccc2-c2ccc(N(c3ccccc3-c3ccc4c(c3)-c3cccc5cccc(c35)O4)c3cccc4oc5c6ccccc6ccc5c34)cc21 ; CC1(c2ccccc2)c2ccccc2-c2ccc(N(c3cccc(-c4ccc5c(c4)-c4cccc6cccc(c46)O5)c3)c3cccc4oc5c6ccccc6ccc5c34)cc21 ; CC1(c2ccccc2)c2ccccc2-c2ccc(N(c3cccc(-c4ccc5c(c4)-c4cccc6cccc(c46)S5)c3)c3cccc4oc5c6ccccc6ccc5c34)cc21]

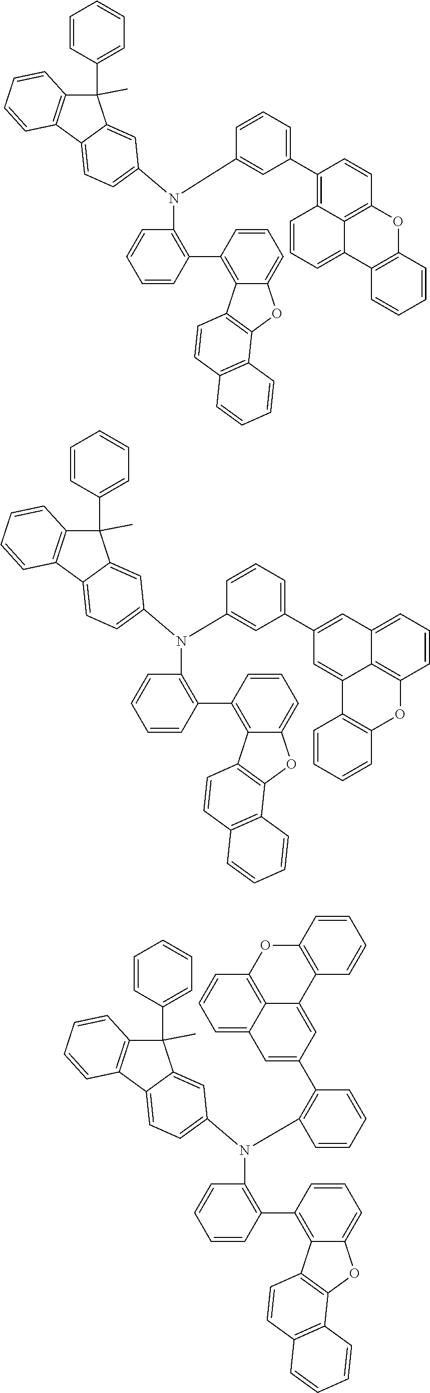 [SMILES: CC1(c2ccccc2)c2ccccc2-c2ccc(N(c3cccc(-c4ccc5c6c(cccc46)-c4ccccc4O5)c3)c3ccccc3-c3cccc4oc5c6ccccc6ccc5c34)cc21 ; CC1(c2ccccc2)c2ccccc2-c2ccc(N(c3cccc(-c4cc5c6c(cccc6c4)Oc4ccccc4-5)c3)c3ccccc3-c3cccc4oc5c6ccccc6ccc5c34)cc21 ; CC1(c2ccccc2)c2ccccc2-c2ccc(N(c3ccccc3-c3cc4c5c(cccc5c3)Oc3ccccc3-4)c3ccccc3-c3cccc4oc5c6ccccc6ccc5c34)cc21]

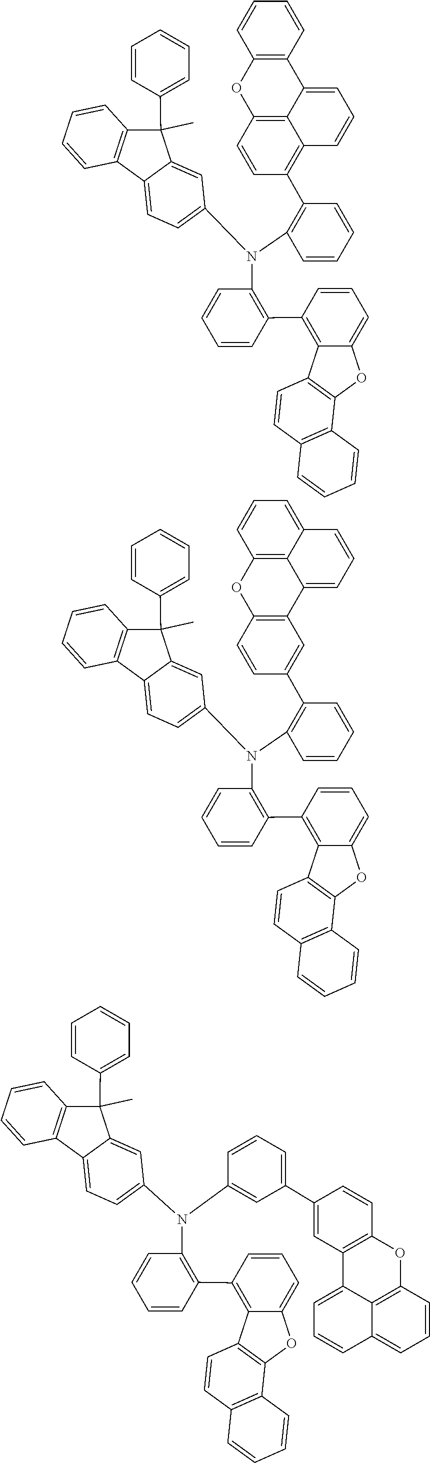

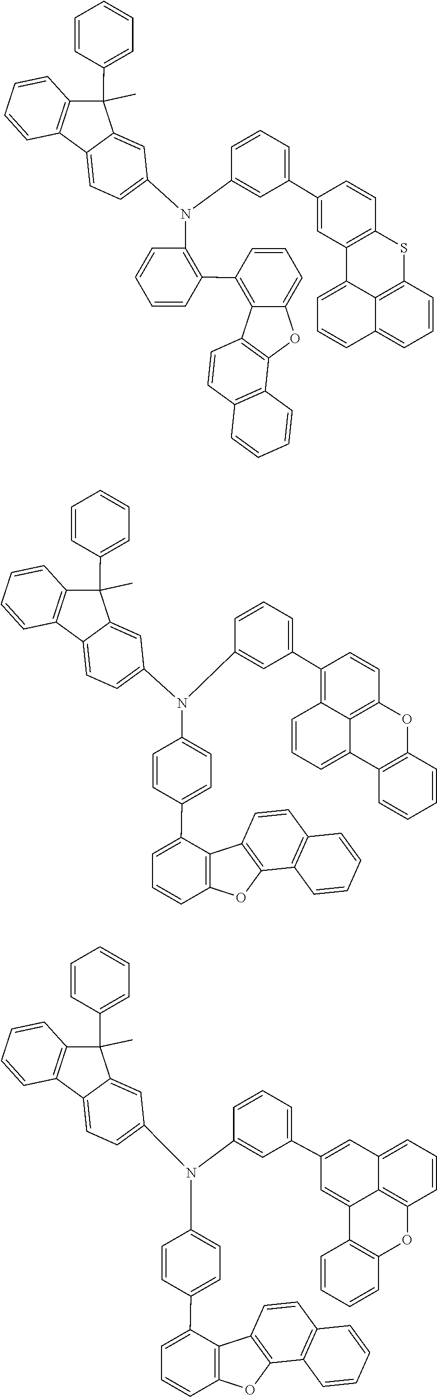 [SMILES: CC1(c2ccccc2)c2ccccc2-c2ccc(N(c3cccc(-c4ccc5c(c4)-c4cccc6cccc(c46)S5)c3)c3ccccc3-c3cccc4oc5c6ccccc6ccc5c34)cc21 ; CC1(c2ccccc2)c2ccccc2-c2ccc(N(c3ccc(-c4cccc5oc6c7ccccc7ccc6c45)cc3)c3cccc(-c4ccc5c6c(cccc46)-c4ccccc4O5)c3)cc21 ; CC1(c2ccccc2)c2ccccc2-c2ccc(N(c3ccc(-c4cccc5oc6c7ccccc7ccc6c45)cc3)c3cccc(-c4cc5c6c(cccc6c4)Oc4ccccc4-5)c3)cc21]

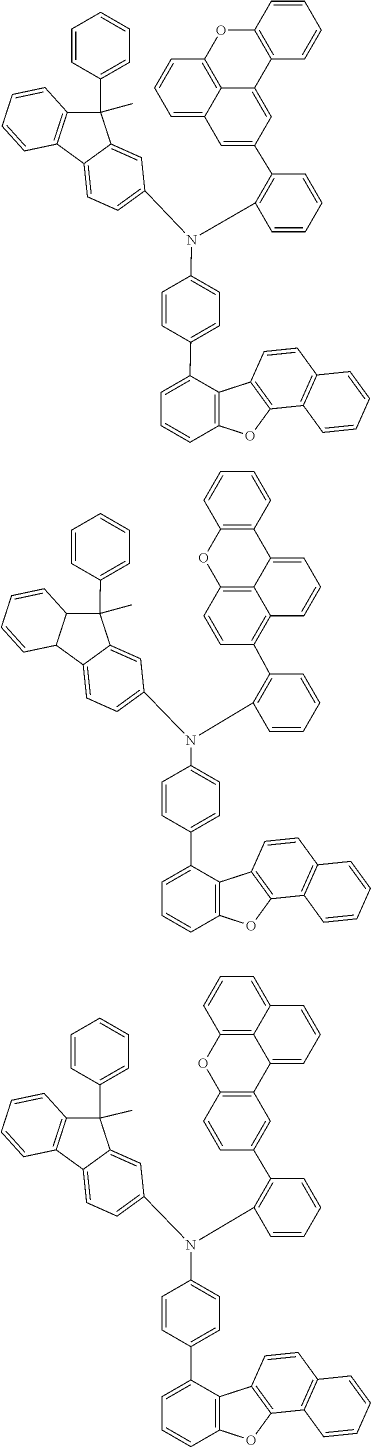

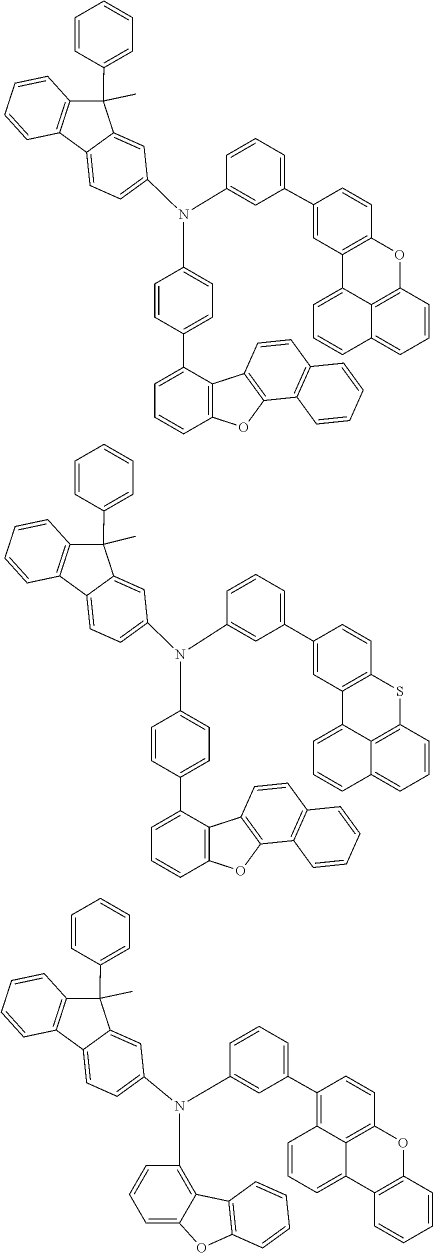 [SMILES: CC1(c2ccccc2)c2ccccc2-c2ccc(N(c3ccc(-c4cccc5oc6c7ccccc7ccc6c45)cc3)c3cccc(-c4ccc5c(c4)-c4cccc6cccc(c46)O5)c3)cc21 ; CC1(c2ccccc2)c2ccccc2-c2ccc(N(c3ccc(-c4cccc5oc6c7ccccc7ccc6c45)cc3)c3cccc(-c4ccc5c(c4)-c4cccc6cccc(c46)S5)c3)cc21 ; CC1(c2ccccc2)c2ccccc2-c2ccc(N(c3cccc(-c4ccc5c6c(cccc46)-c4ccccc4O5)c3)c3cccc4oc5ccccc5c34)cc21]

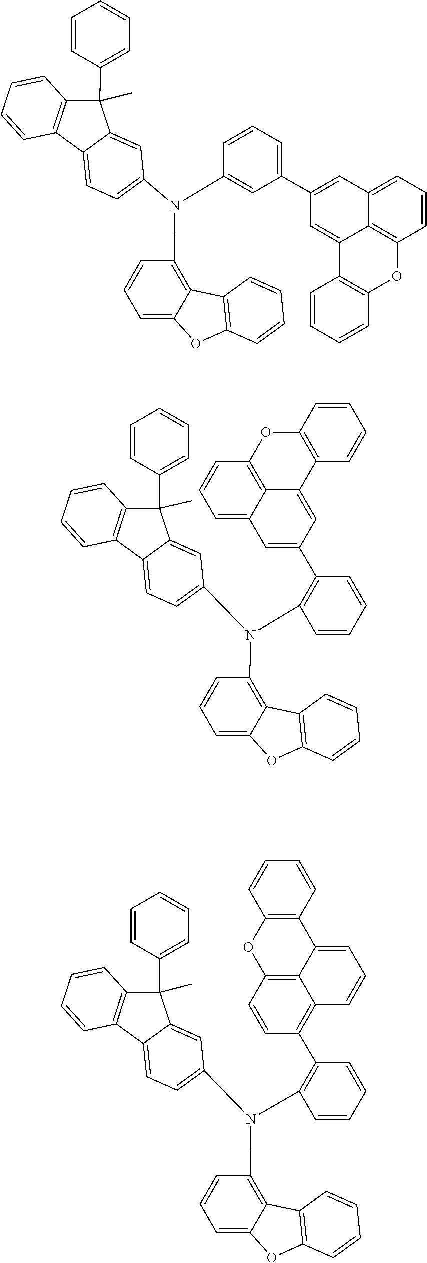 [SMILES: CC1(c2ccccc2)c2ccccc2-c2ccc(N(c3cccc(-c4cc5c6c(cccc6c4)Oc4ccccc4-5)c3)c3cccc4oc5ccccc5c34)cc21 ; CC1(c2ccccc2)c2ccccc2-c2ccc(N(c3ccccc3-c3cc4c5c(cccc5c3)Oc3ccccc3-4)c3cccc4oc5ccccc5c34)cc21 ; CC1(c2ccccc2)c2ccccc2-c2ccc(N(c3ccccc3-c3ccc4c5c(cccc35)-c3ccccc3O4)c3cccc4oc5ccccc5c34)cc21]

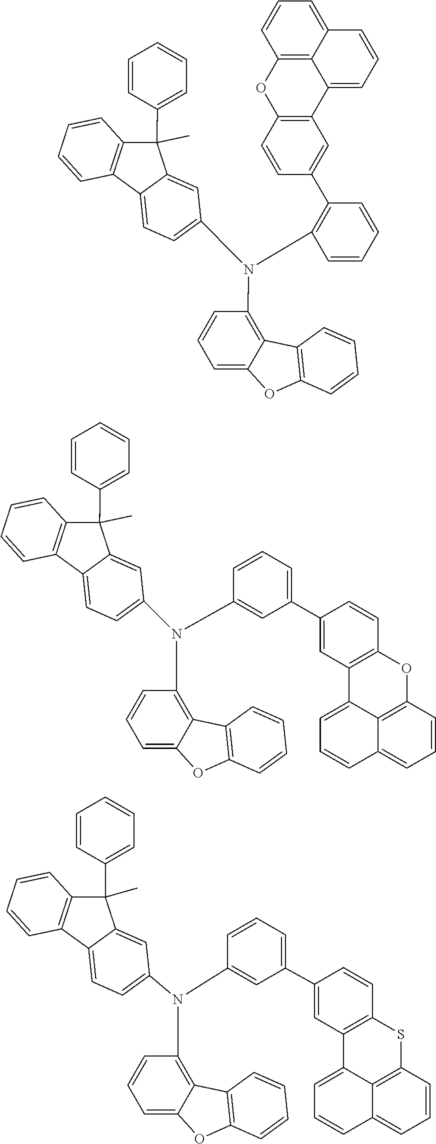 [SMILES: CC1(c2ccccc2)c2ccccc2-c2ccc(N(c3ccccc3-c3ccc4c(c3)-c3cccc5cccc(c35)O4)c3cccc4oc5ccccc5c34)cc21 ; CC1(c2ccccc2)c2ccccc2-c2ccc(N(c3cccc(-c4ccc5c(c4)-c4cccc6cccc(c46)O5)c3)c3cccc4oc5ccccc5c34)cc21 ; CC1(c2ccccc2)c2ccccc2-c2ccc(N(c3cccc(-c4ccc5c(c4)-c4cccc6cccc(c46)S5)c3)c3cccc4oc5ccccc5c34)cc21]

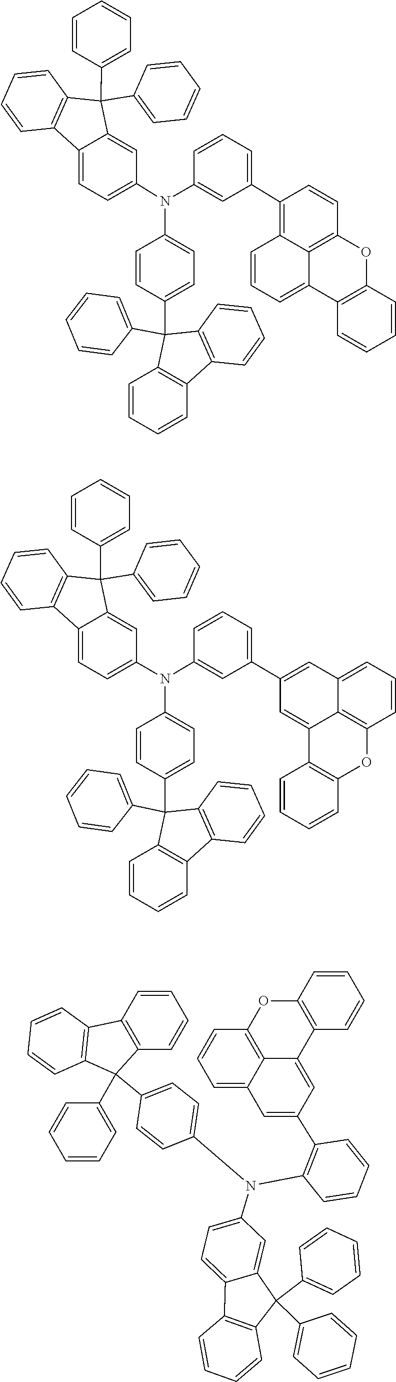 [SMILES: c1ccc(C2(c3ccc(N(c4cccc(-c5ccc6c7c(cccc57)-c5ccccc5O6)c4)c4ccc5c(c4)C(c4ccccc4)(c4ccccc4)c4ccccc4-5)cc3)c3ccccc3-c3ccccc32)cc1 ; c1ccc(C2(c3ccc(N(c4cccc(-c5cc6c7c(cccc7c5)Oc5ccccc5-6)c4)c4ccc5c(c4)C(c4ccccc4)(c4ccccc4)c4ccccc4-5)cc3)c3ccccc3-c3ccccc32)cc1 ; c1ccc(C2(c3ccc(N(c4ccc5c(c4)C(c4ccccc4)(c4ccccc4)c4ccccc4-5)c4ccccc4-c4cc5c6c(cccc6c4)Oc4ccccc4-5)cc3)c3ccccc3-c3ccccc32)cc1]

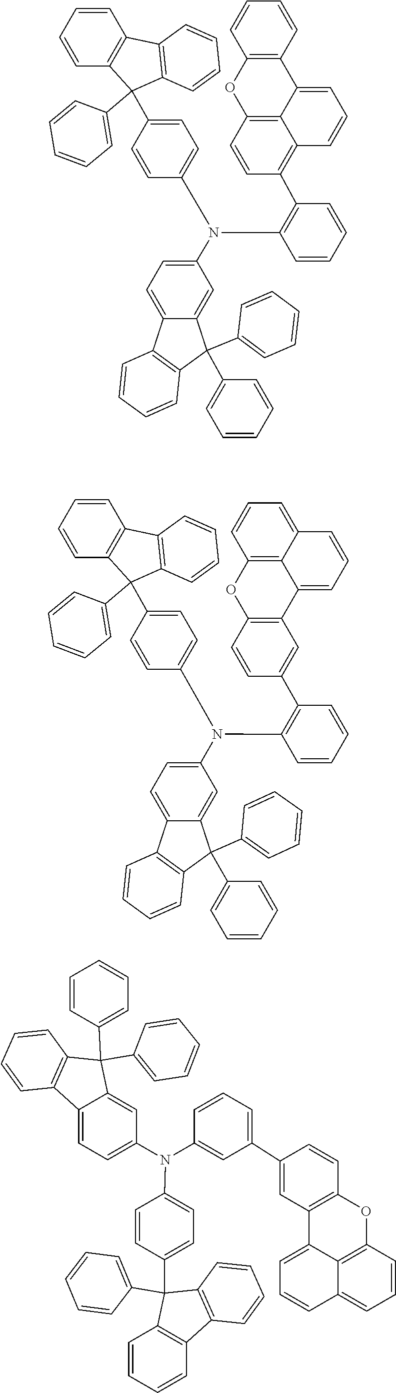 [SMILES: c1ccc(C2(c3ccc(N(c4ccc5c(c4)C(c4ccccc4)(c4ccccc4)c4ccccc4-5)c4ccccc4-c4ccc5c6c(cccc46)-c4ccccc4O5)cc3)c3ccccc3-c3ccccc32)cc1 ; c1ccc(C2(c3ccc(N(c4ccc5c(c4)C(c4ccccc4)(c4ccccc4)c4ccccc4-5)c4ccccc4-c4ccc5c(c4)-c4cccc6cccc(c46)O5)cc3)c3ccccc3-c3ccccc32)cc1 ; c1ccc(C2(c3ccc(N(c4cccc(-c5ccc6c(c5)-c5cccc7cccc(c57)O6)c4)c4ccc5c(c4)C(c4ccccc4)(c4ccccc4)c4ccccc4-5)cc3)c3ccccc3-c3ccccc32)cc1]

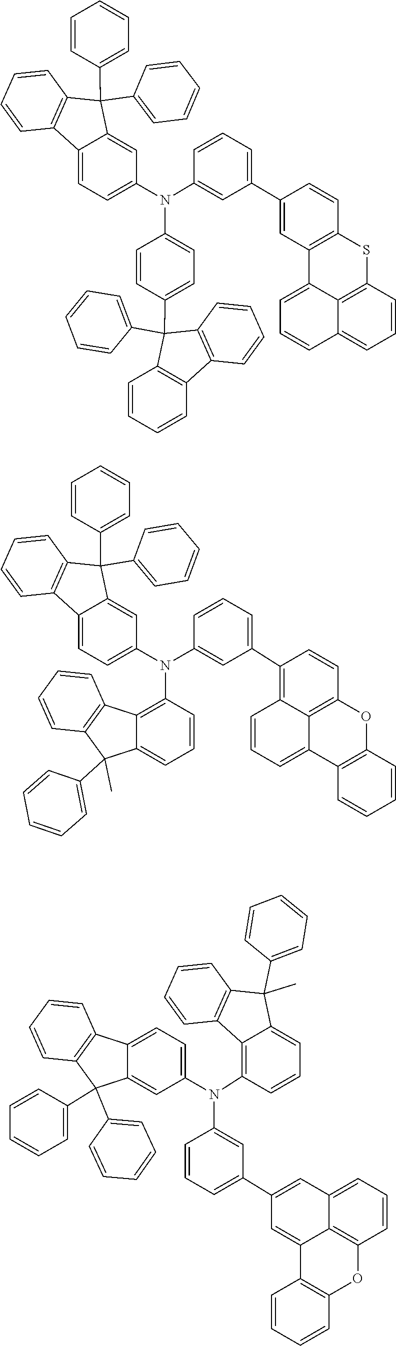 [SMILES: c1ccc(C2(c3ccc(N(c4cccc(-c5ccc6c(c5)-c5cccc7cccc(c57)S6)c4)c4ccc5c(c4)C(c4ccccc4)(c4ccccc4)c4ccccc4-5)cc3)c3ccccc3-c3ccccc32)cc1 ; CC1(c2ccccc2)c2ccccc2-c2c(N(c3cccc(-c4ccc5c6c(cccc46)-c4ccccc4O5)c3)c3ccc4c(c3)C(c3ccccc3)(c3ccccc3)c3ccccc3-4)cccc21 ; CC1(c2ccccc2)c2ccccc2-c2c(N(c3cccc(-c4cc5c6c(cccc6c4)Oc4ccccc4-5)c3)c3ccc4c(c3)C(c3ccccc3)(c3ccccc3)c3ccccc3-4)cccc21]

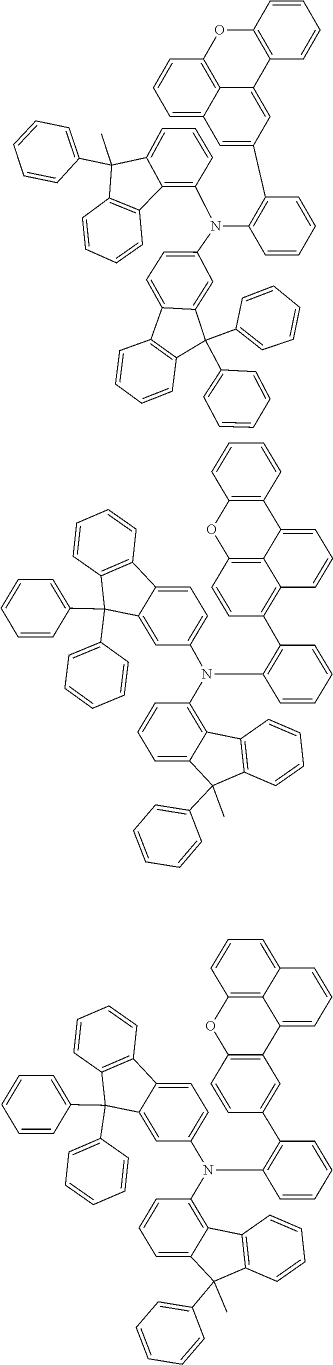

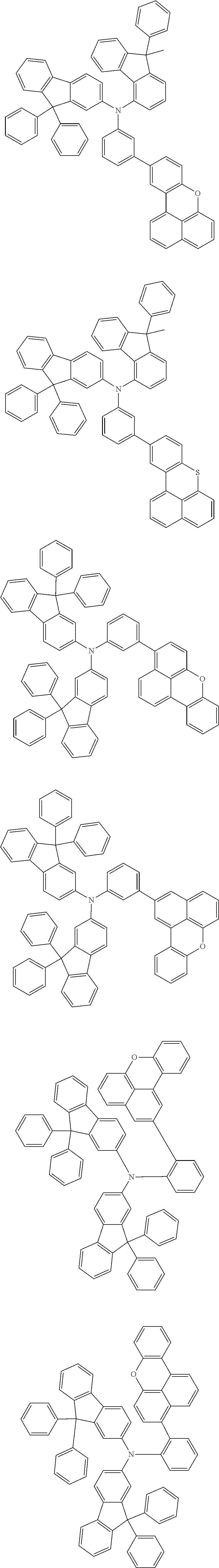

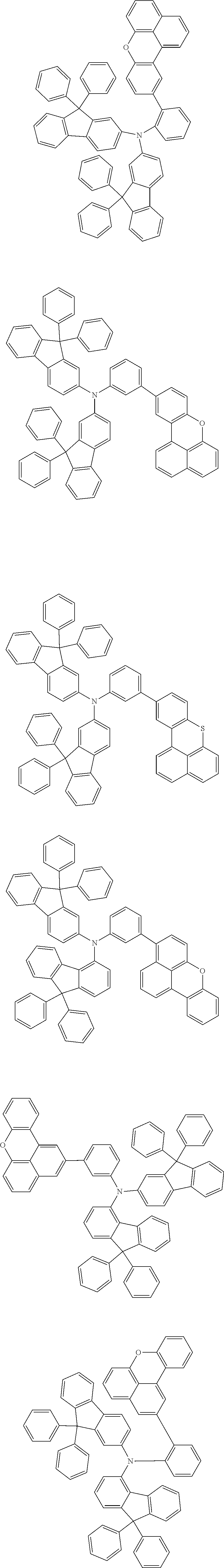

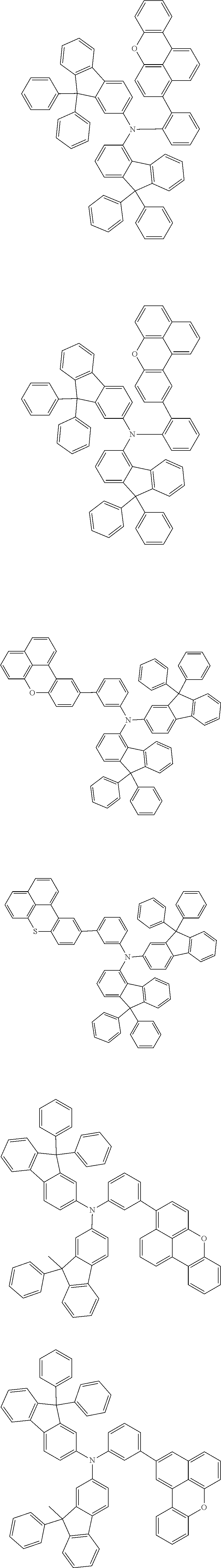

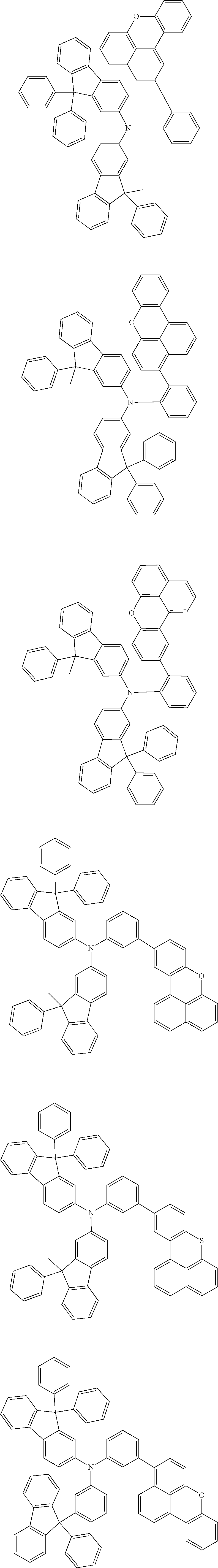

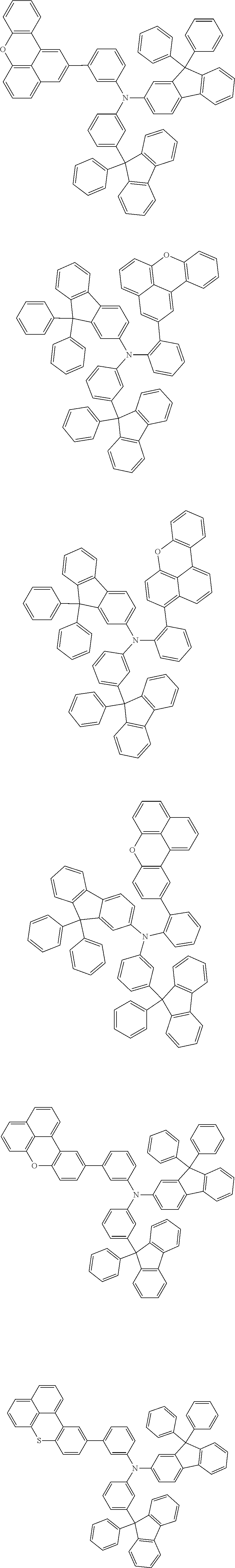

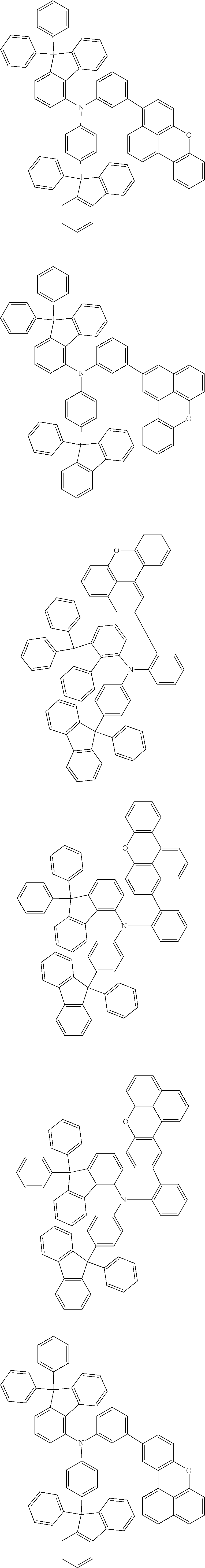

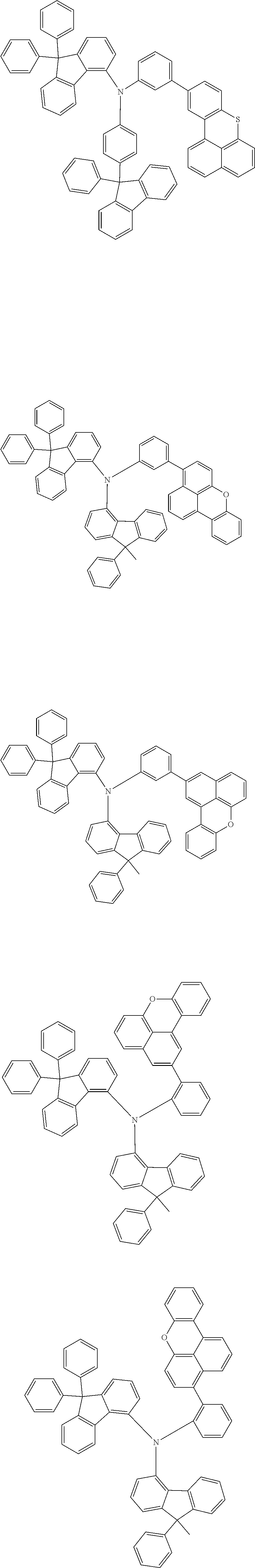

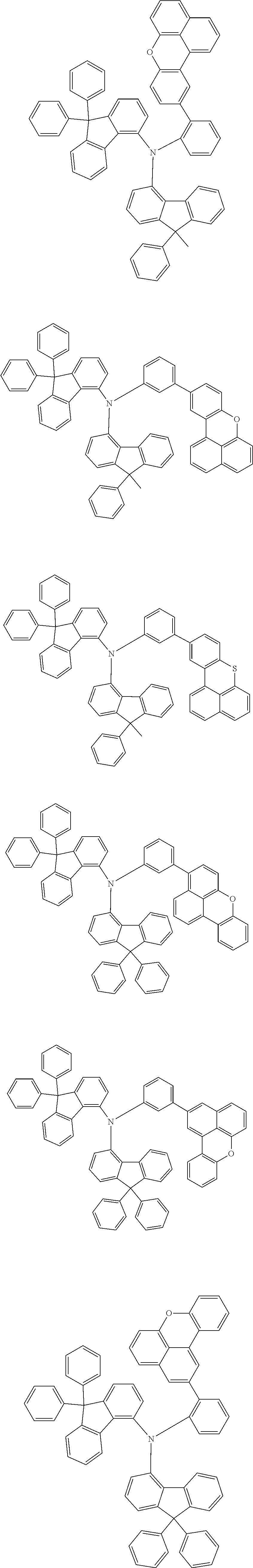

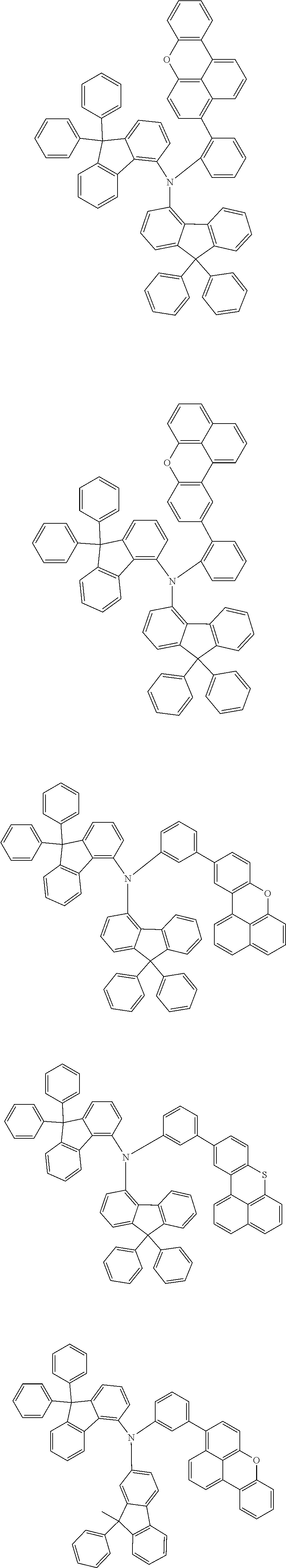

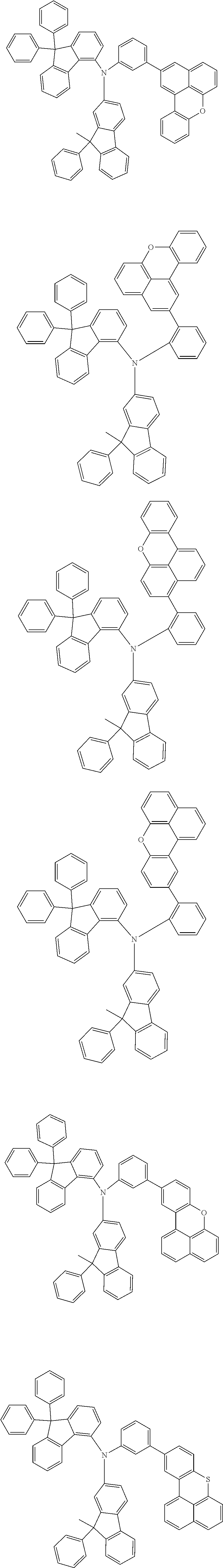

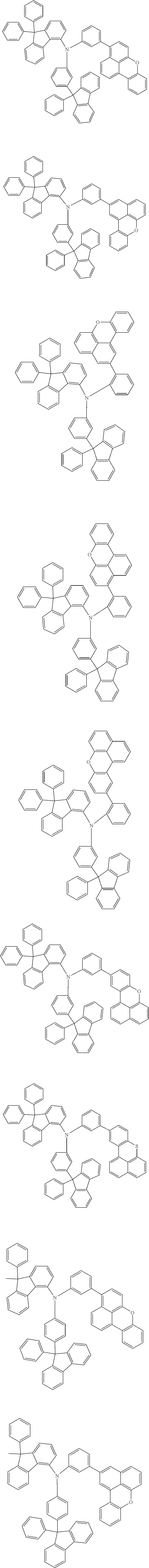

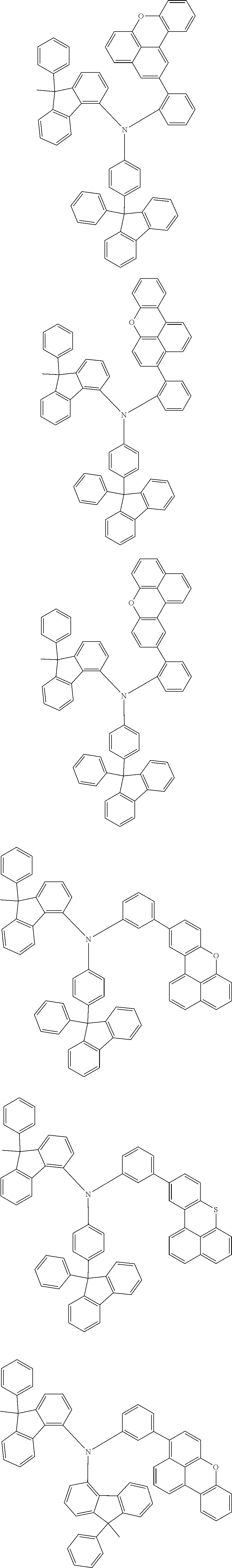

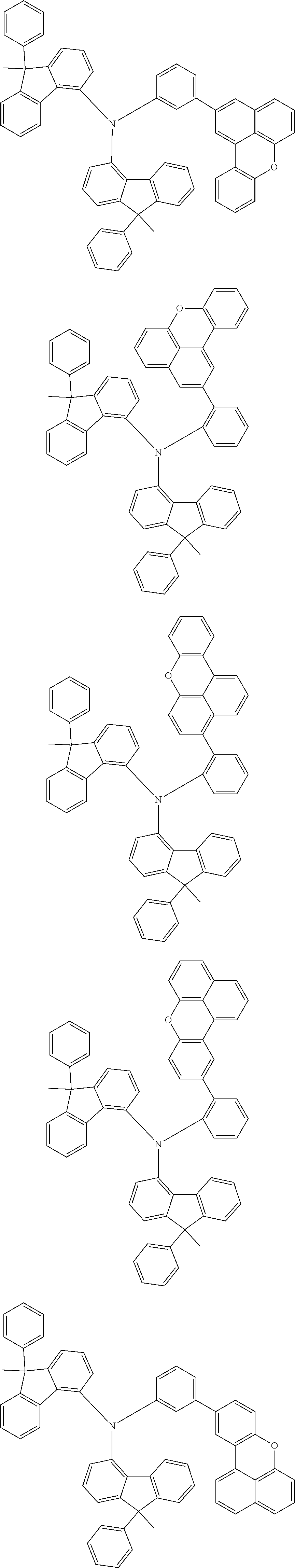

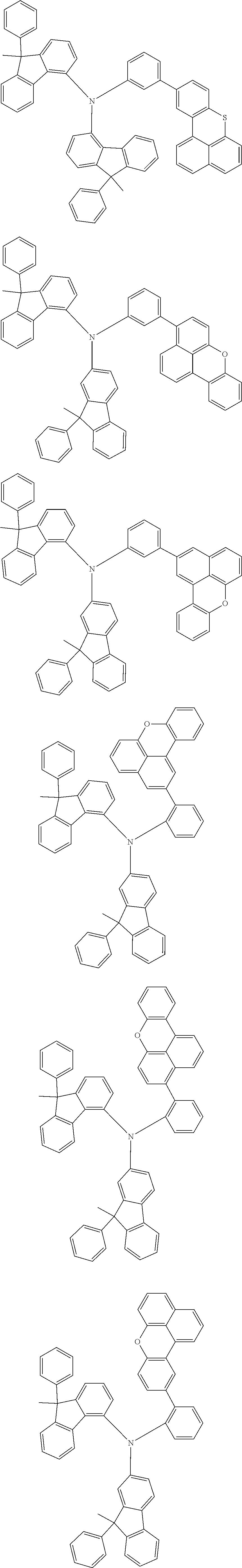

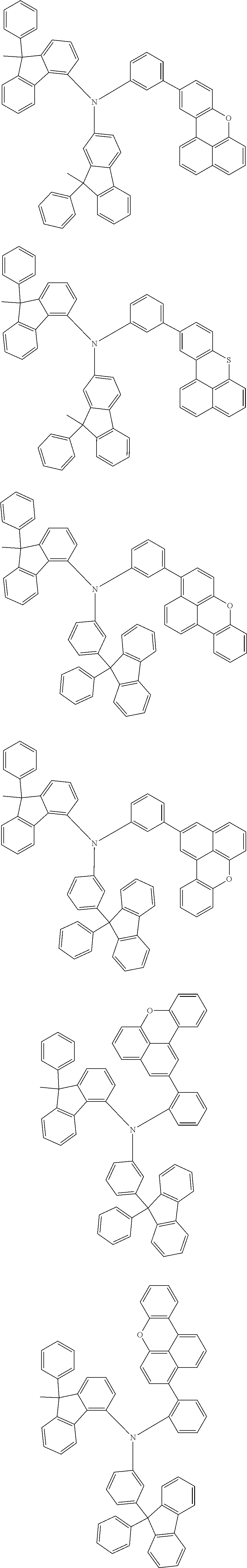

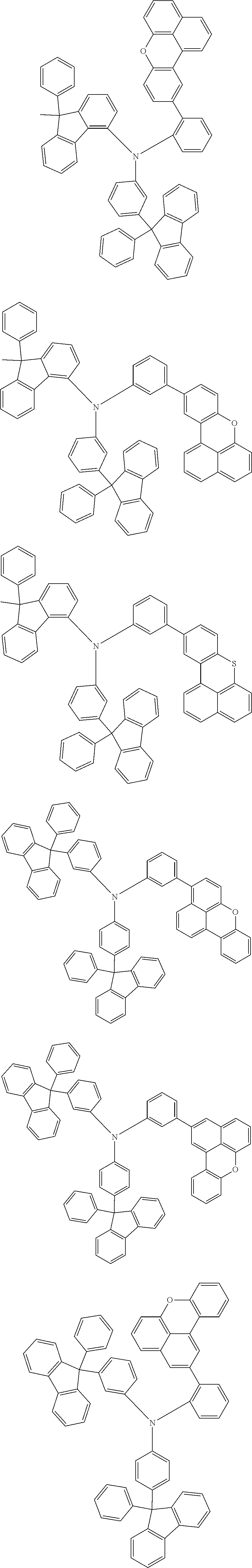

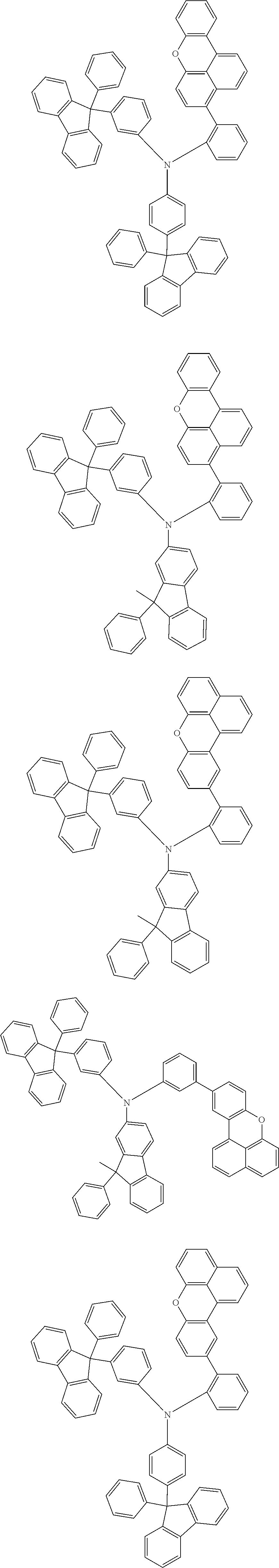

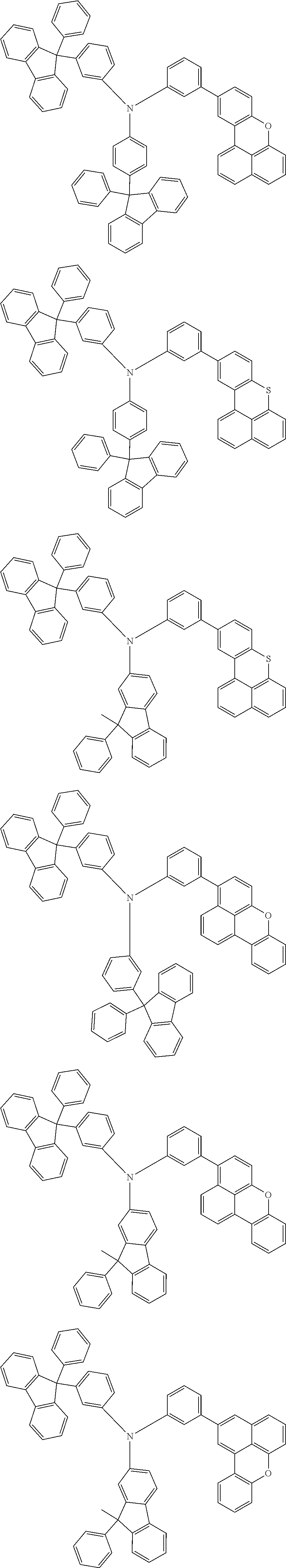

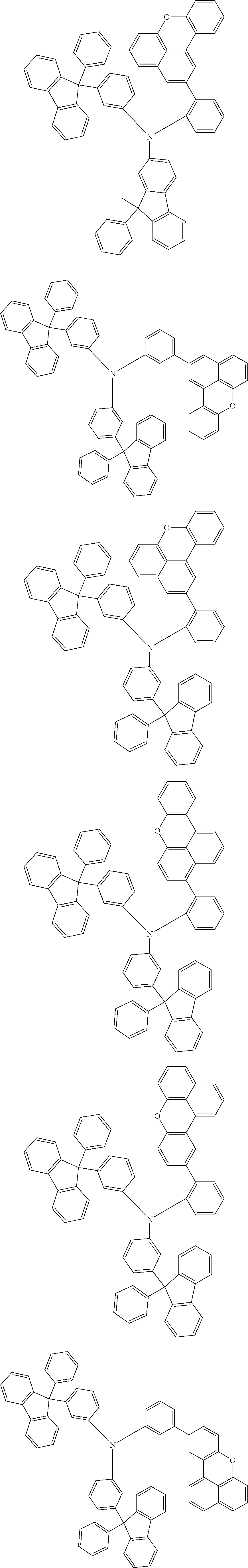

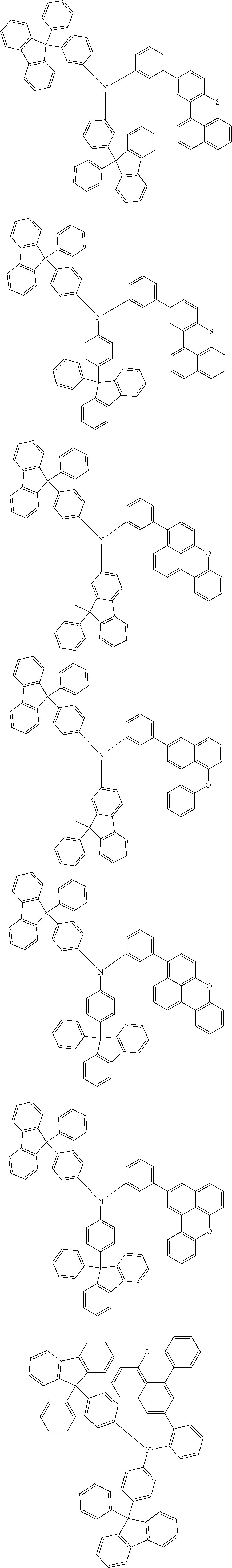

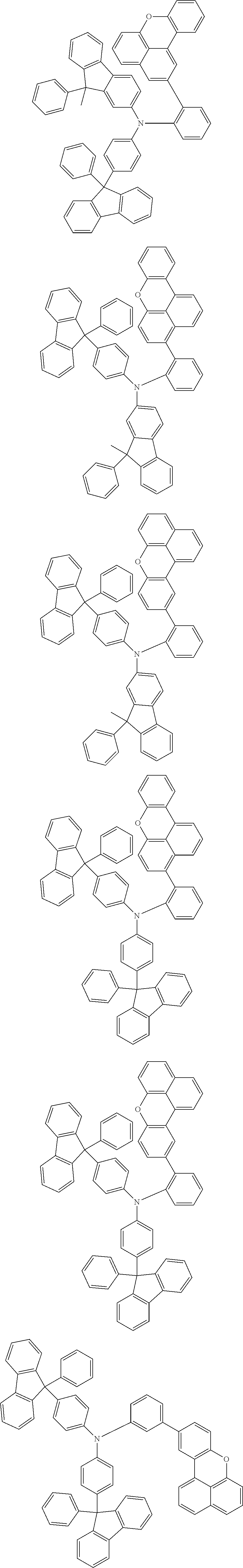

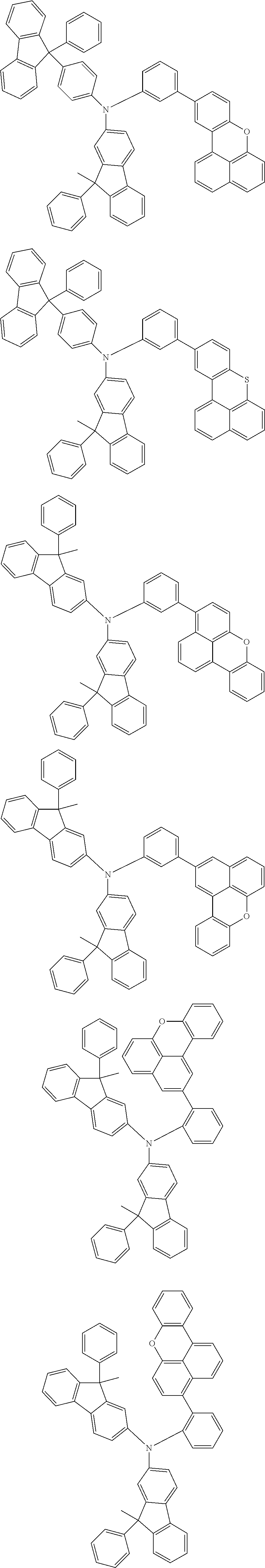

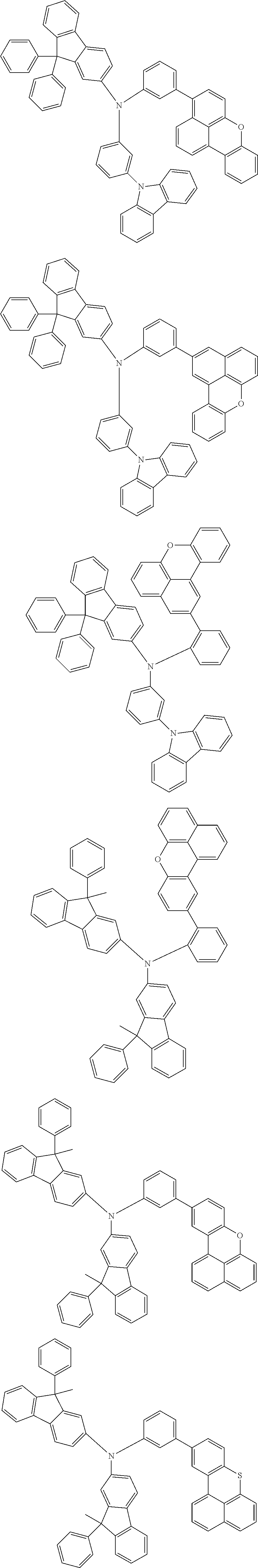

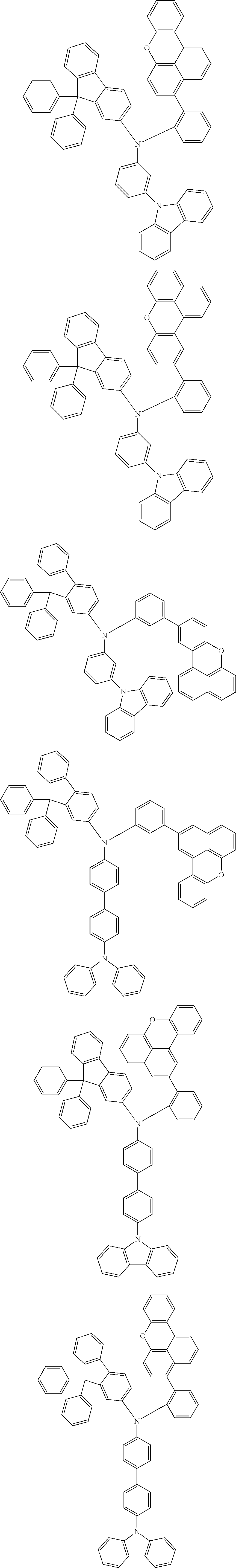

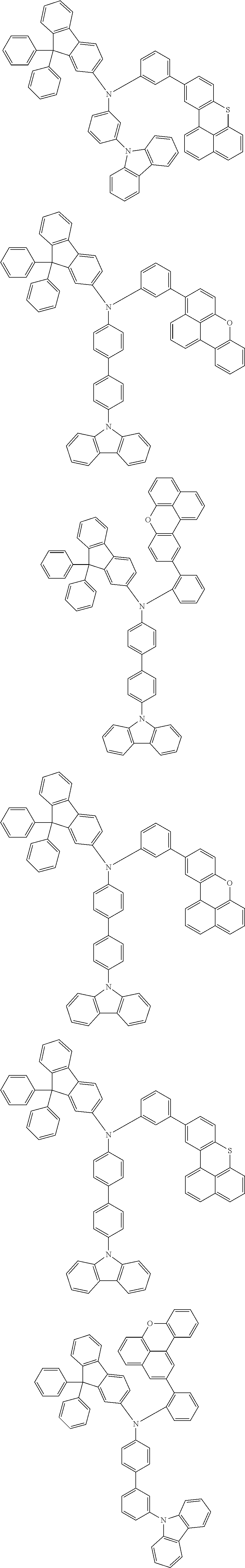

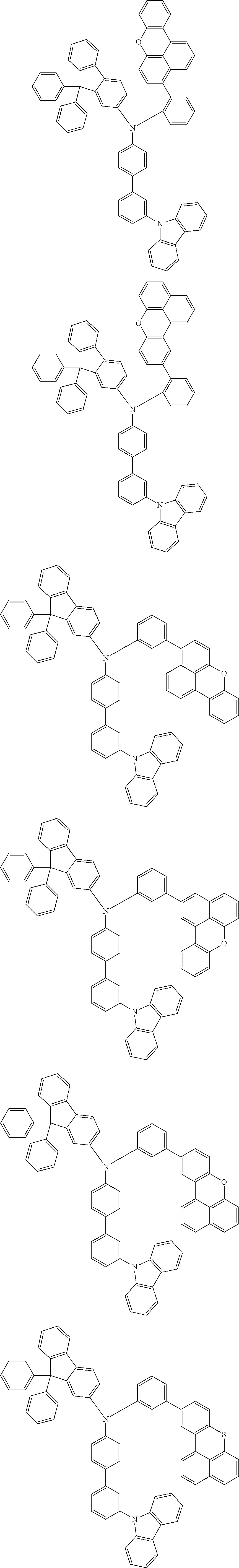

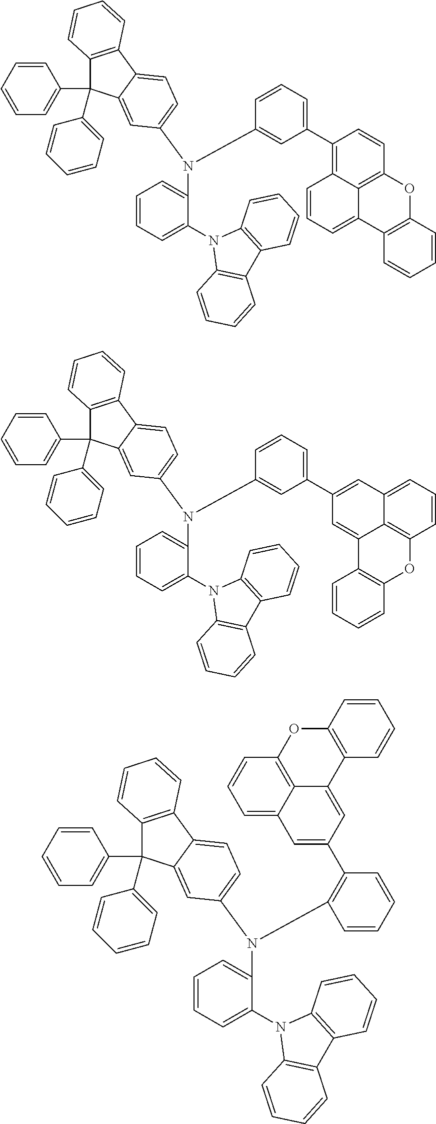 [SMILES: c1ccc(C2(c3ccccc3)c3ccccc3-c3ccc(N(c4cccc(-c5ccc6c7c(cccc57)-c5ccccc5O6)c4)c4ccccc4-n4c5ccccc5c5ccccc54)cc32)cc1 ; c1ccc(C2(c3ccccc3)c3ccccc3-c3ccc(N(c4cccc(-c5cc6c7c(cccc7c5)Oc5ccccc5-6)c4)c4ccccc4-n4c5ccccc5c5ccccc54)cc32)cc1 ; c1ccc(C2(c3ccccc3)c3ccccc3-c3ccc(N(c4ccccc4-c4cc5c6c(cccc6c4)Oc4ccccc4-5)c4ccccc4-n4c5ccccc5c5ccccc54)cc32)cc1]

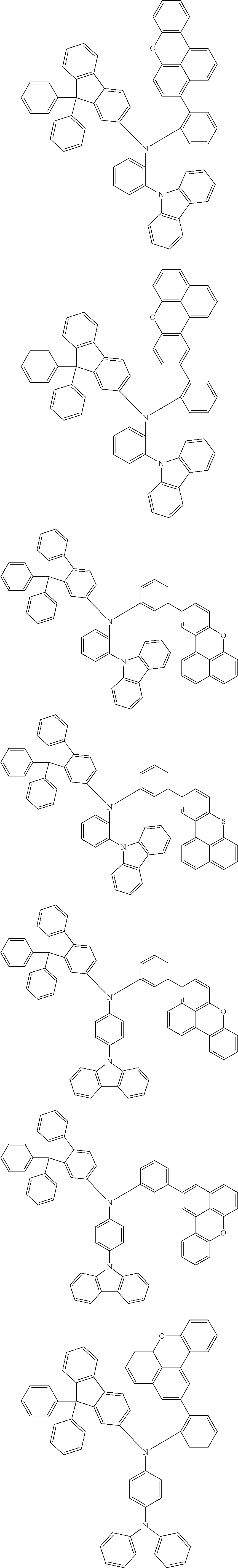

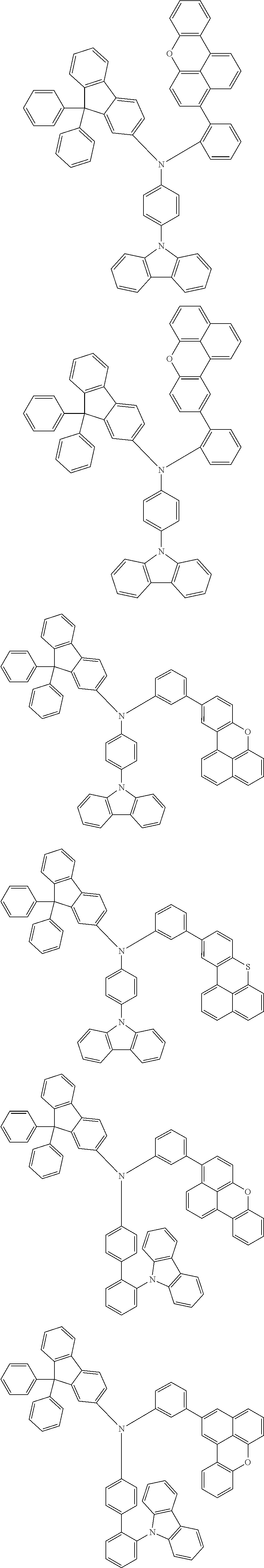

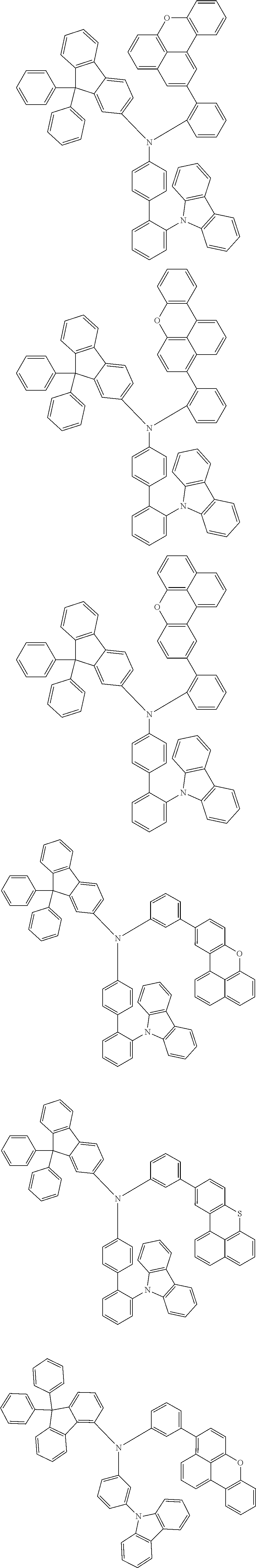

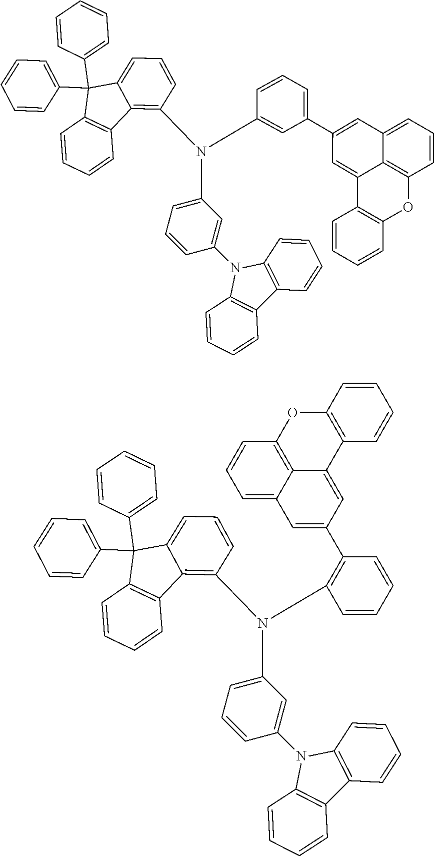 [SMILES: c1ccc(C2(c3ccccc3)c3ccccc3-c3c(N(c4cccc(-c5cc6c7c(cccc7c5)Oc5ccccc5-6)c4)c4cccc(-n5c6ccccc6c6ccccc65)c4)cccc32)cc1 ; c1ccc(C2(c3ccccc3)c3ccccc3-c3c(N(c4cccc(-n5c6ccccc6c6ccccc65)c4)c4ccccc4-c4cc5c6c(cccc6c4)Oc4ccccc4-5)cccc32)cc1]

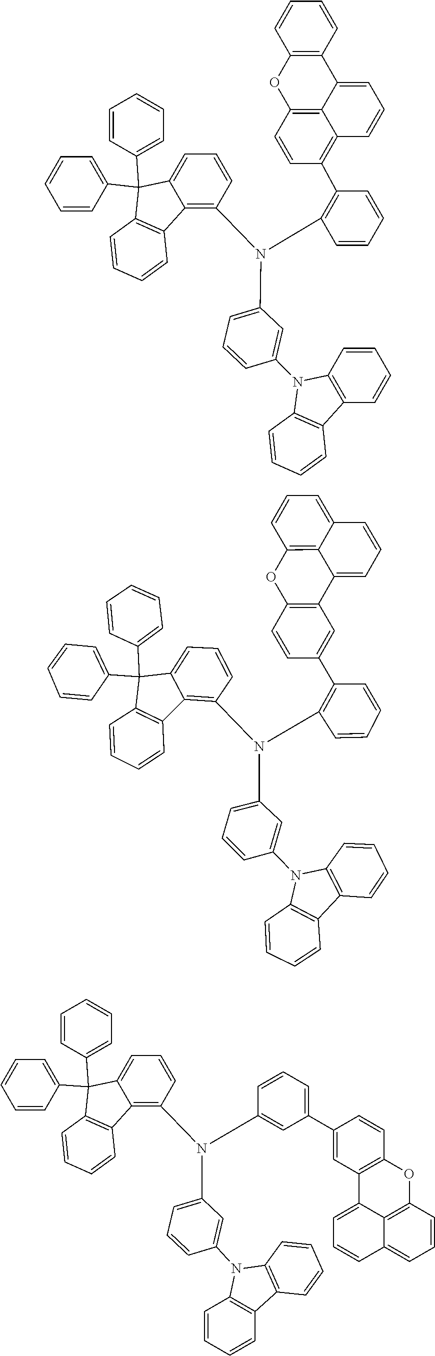

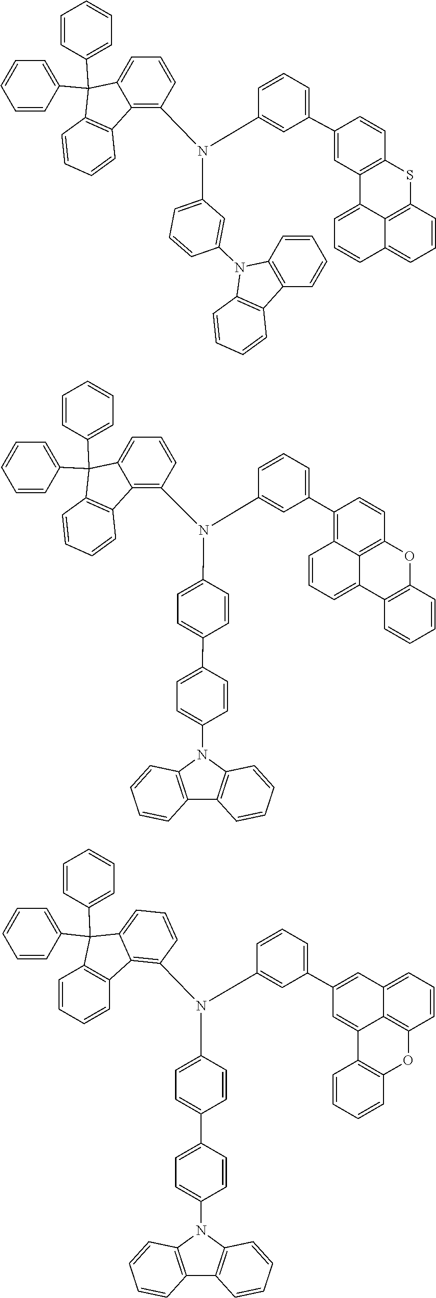 [SMILES: c1ccc(C2(c3ccccc3)c3ccccc3-c3c(N(c4cccc(-c5ccc6c(c5)-c5cccc7cccc(c57)S6)c4)c4cccc(-n5c6ccccc6c6ccccc65)c4)cccc32)cc1 ; c1ccc(C2(c3ccccc3)c3ccccc3-c3c(N(c4ccc(-c5ccc(-n6c7ccccc7c7ccccc76)cc5)cc4)c4cccc(-c5ccc6c7c(cccc57)-c5ccccc5O6)c4)cccc32)cc1 ; c1ccc(C2(c3ccccc3)c3ccccc3-c3c(N(c4ccc(-c5ccc(-n6c7ccccc7c7ccccc76)cc5)cc4)c4cccc(-c5cc6c7c(cccc7c5)Oc5ccccc5-6)c4)cccc32)cc1]

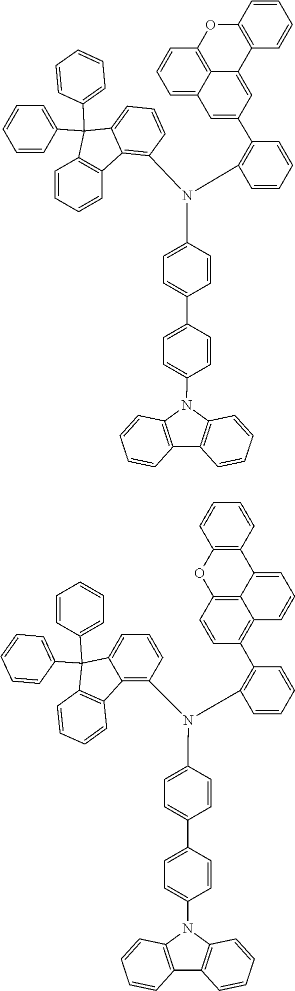

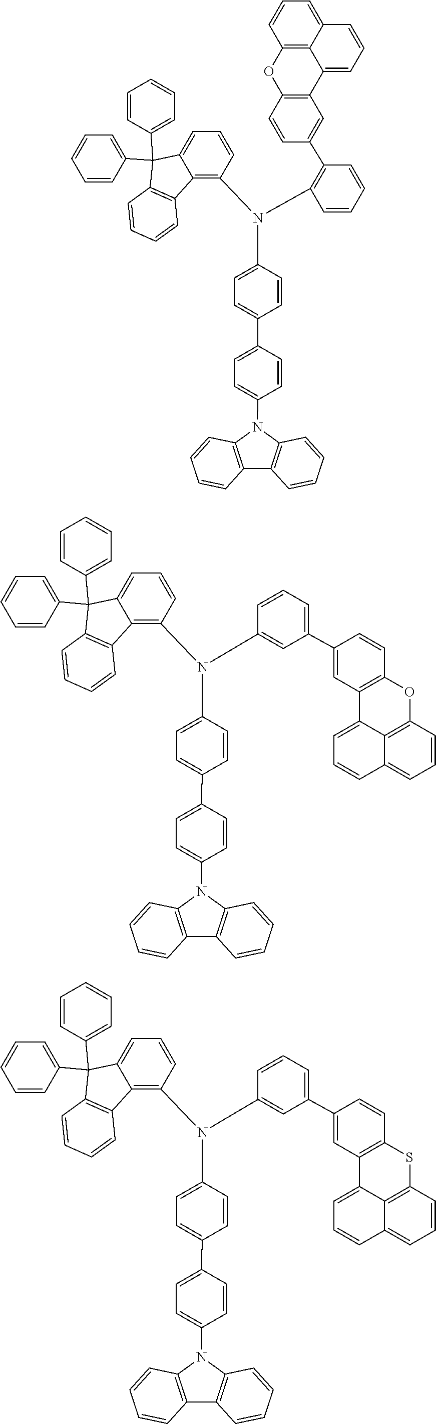 [SMILES: c1ccc(C2(c3ccccc3)c3ccccc3-c3c(N(c4ccc(-c5ccc(-n6c7ccccc7c7ccccc76)cc5)cc4)c4ccccc4-c4ccc5c(c4)-c4cccc6cccc(c46)O5)cccc32)cc1 ; c1ccc(C2(c3ccccc3)c3ccccc3-c3c(N(c4ccc(-c5ccc(-n6c7ccccc7c7ccccc76)cc5)cc4)c4cccc(-c5ccc6c(c5)-c5cccc7cccc(c57)O6)c4)cccc32)cc1 ; c1ccc(C2(c3ccccc3)c3ccccc3-c3c(N(c4ccc(-c5ccc(-n6c7ccccc7c7ccccc76)cc5)cc4)c4cccc(-c5ccc6c(c5)-c5cccc7cccc(c57)S6)c4)cccc32)cc1]

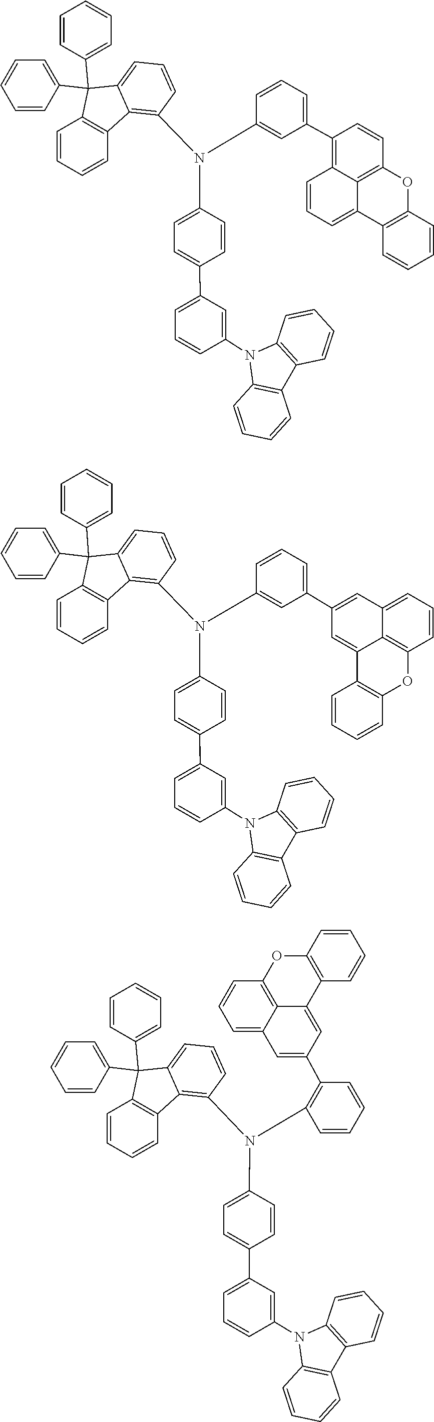 [SMILES: c1ccc(C2(c3ccccc3)c3ccccc3-c3c(N(c4ccc(-c5cccc(-n6c7ccccc7c7ccccc76)c5)cc4)c4cccc(-c5ccc6c7c(cccc57)-c5ccccc5O6)c4)cccc32)cc1 ; c1ccc(C2(c3ccccc3)c3ccccc3-c3c(N(c4ccc(-c5cccc(-n6c7ccccc7c7ccccc76)c5)cc4)c4cccc(-c5cc6c7c(cccc7c5)Oc5ccccc5-6)c4)cccc32)cc1 ; c1ccc(C2(c3ccccc3)c3ccccc3-c3c(N(c4ccc(-c5cccc(-n6c7ccccc7c7ccccc76)c5)cc4)c4ccccc4-c4cc5c6c(cccc6c4)Oc4ccccc4-5)cccc32)cc1]

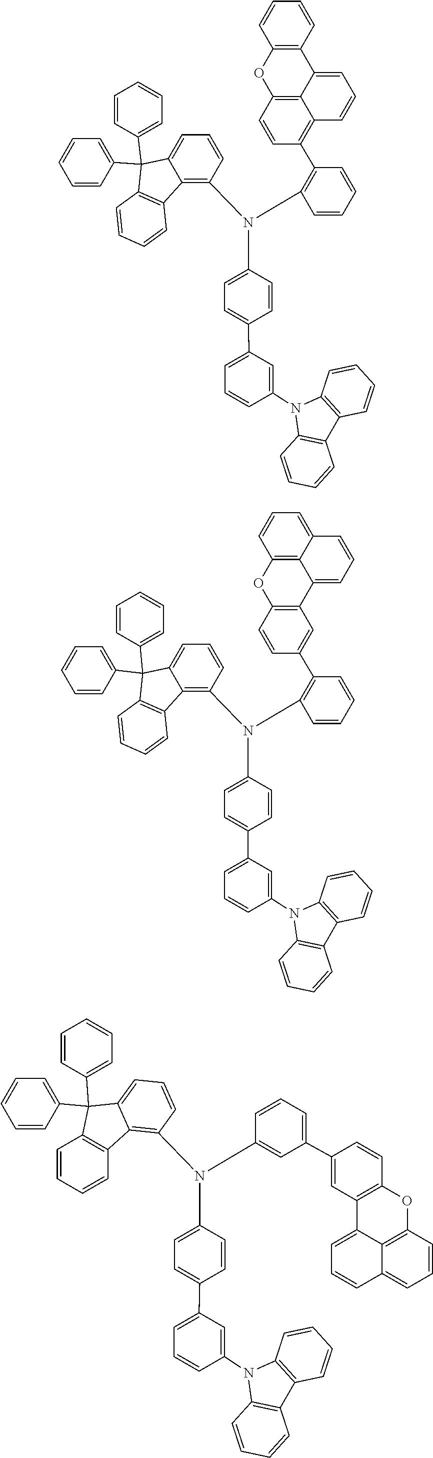

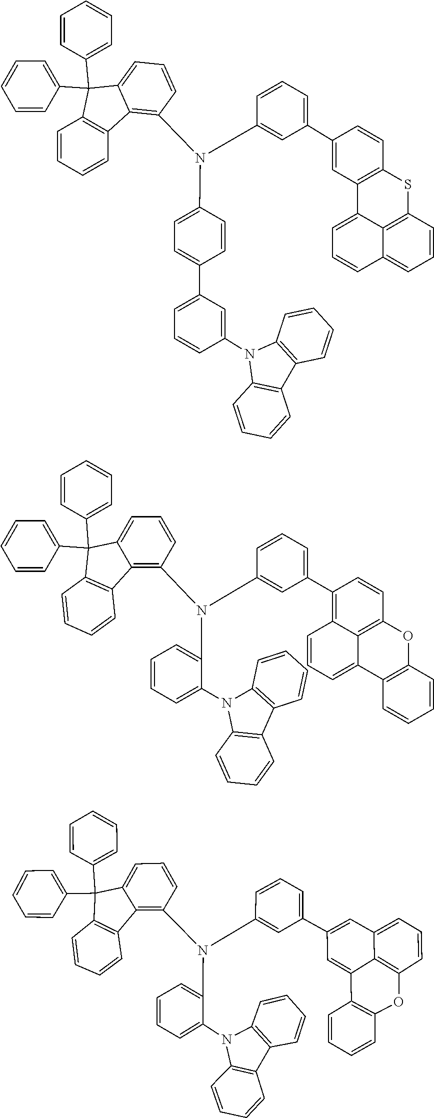 [SMILES: c1ccc(C2(c3ccccc3)c3ccccc3-c3c(N(c4ccc(-c5cccc(-n6c7ccccc7c7ccccc76)c5)cc4)c4cccc(-c5ccc6c(c5)-c5cccc7cccc(c57)S6)c4)cccc32)cc1 ; c1ccc(C2(c3ccccc3)c3ccccc3-c3c(N(c4cccc(-c5ccc6c7c(cccc57)-c5ccccc5O6)c4)c4ccccc4-n4c5ccccc5c5ccccc54)cccc32)cc1 ; c1ccc(C2(c3ccccc3)c3ccccc3-c3c(N(c4cccc(-c5cc6c7c(cccc7c5)Oc5ccccc5-6)c4)c4ccccc4-n4c5ccccc5c5ccccc54)cccc32)cc1]

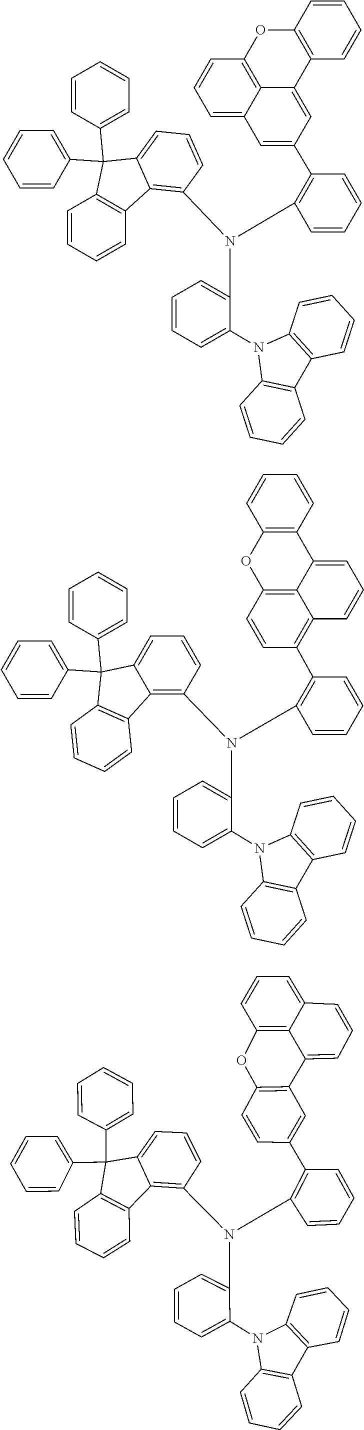

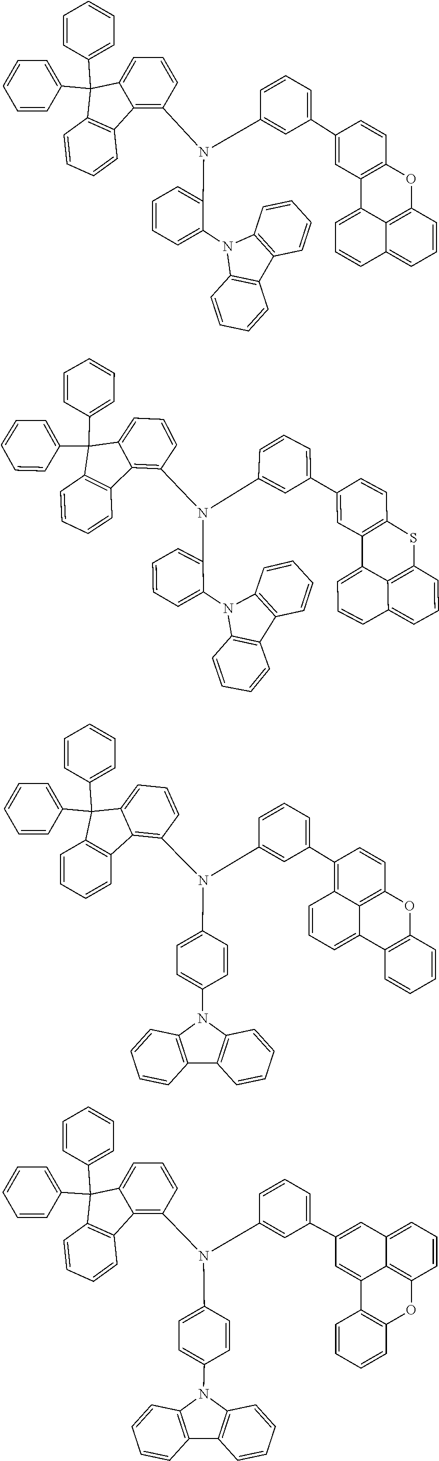 [SMILES: c1ccc(C2(c3ccccc3)c3ccccc3-c3c(N(c4cccc(-c5ccc6c(c5)-c5cccc7cccc(c57)O6)c4)c4ccccc4-n4c5ccccc5c5ccccc54)cccc32)cc1 ; c1ccc(C2(c3ccccc3)c3ccccc3-c3c(N(c4cccc(-c5ccc6c(c5)-c5cccc7cccc(c57)S6)c4)c4ccccc4-n4c5ccccc5c5ccccc54)cccc32)cc1 ; c1ccc(C2(c3ccccc3)c3ccccc3-c3c(N(c4ccc(-n5c6ccccc6c6ccccc65)cc4)c4cccc(-c5ccc6c7c(cccc57)-c5ccccc5O6)c4)cccc32)cc1 ; c1ccc(C2(c3ccccc3)c3ccccc3-c3c(N(c4ccc(-n5c6ccccc6c6ccccc65)cc4)c4cccc(-c5cc6c7c(cccc7c5)Oc5ccccc5-6)c4)cccc32)cc1]

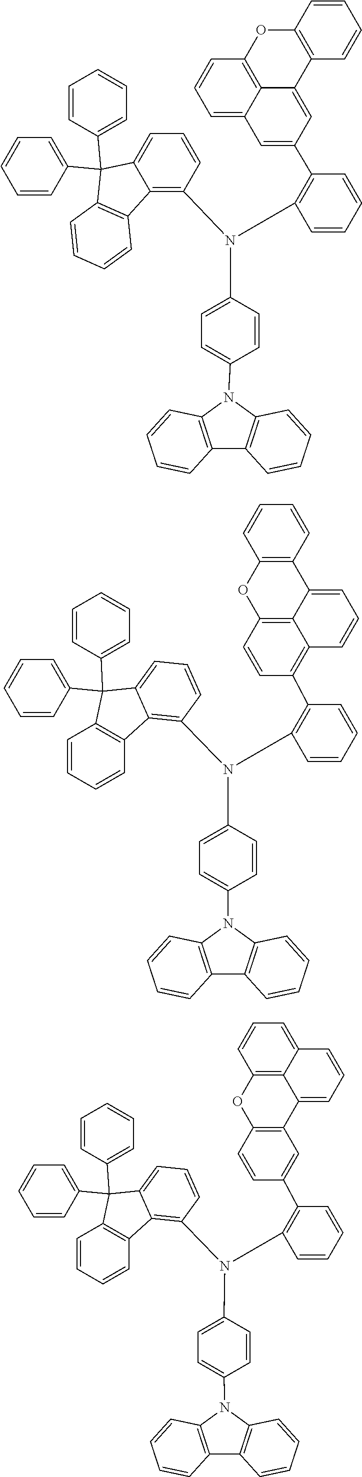

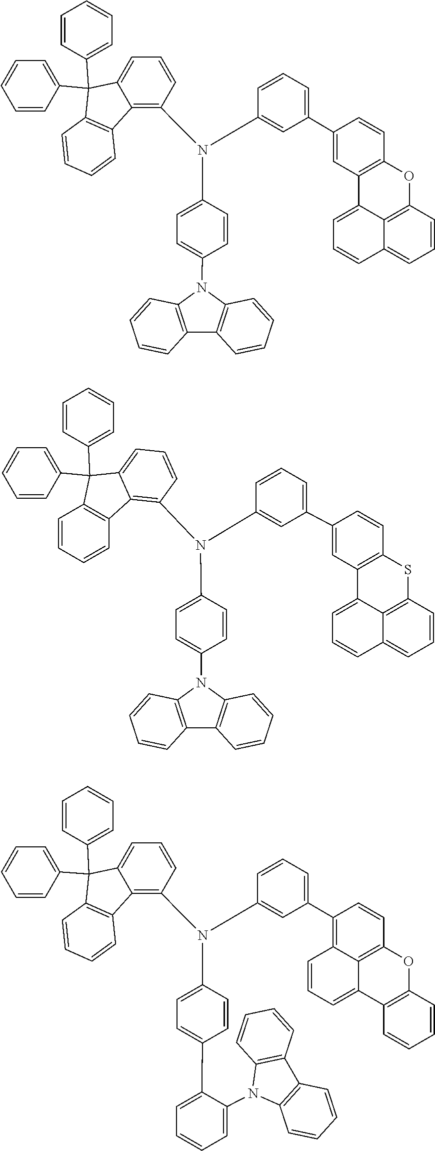 [SMILES: c1ccc(C2(c3ccccc3)c3ccccc3-c3c(N(c4ccc(-n5c6ccccc6c6ccccc65)cc4)c4cccc(-c5ccc6c(c5)-c5cccc7cccc(c57)O6)c4)cccc32)cc1 ; c1ccc(C2(c3ccccc3)c3ccccc3-c3c(N(c4ccc(-n5c6ccccc6c6ccccc65)cc4)c4cccc(-c5ccc6c(c5)-c5cccc7cccc(c57)S6)c4)cccc32)cc1 ; c1ccc(C2(c3ccccc3)c3ccccc3-c3c(N(c4ccc(-c5ccccc5-n5c6ccccc6c6ccccc65)cc4)c4cccc(-c5ccc6c7c(cccc57)-c5ccccc5O6)c4)cccc32)cc1]

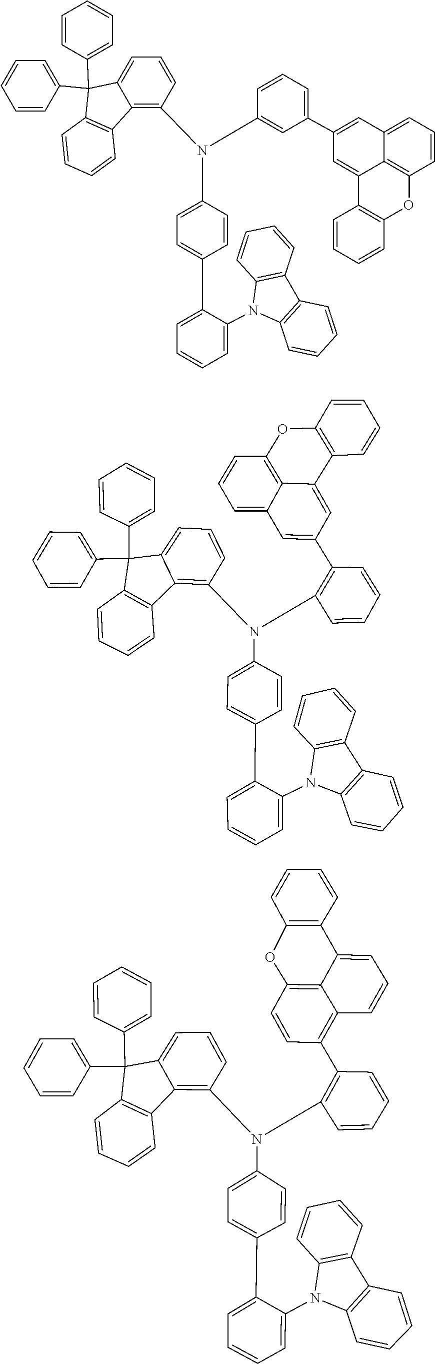

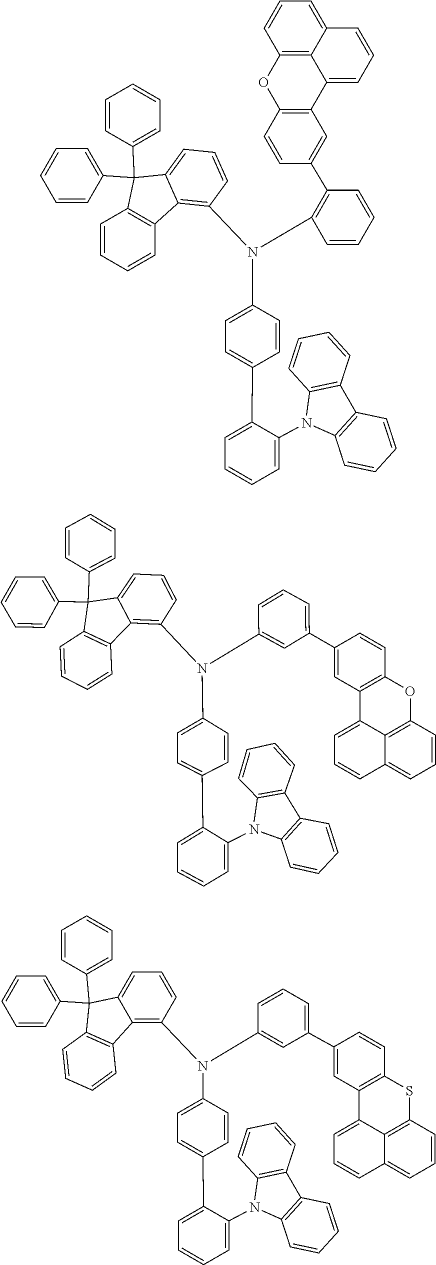 [SMILES: c1ccc(C2(c3ccccc3)c3ccccc3-c3c(N(c4ccc(-c5ccccc5-n5c6ccccc6c6ccccc65)cc4)c4ccccc4-c4ccc5c(c4)-c4cccc6cccc(c46)O5)cccc32)cc1 ; c1ccc(C2(c3ccccc3)c3ccccc3-c3c(N(c4ccc(-c5ccccc5-n5c6ccccc6c6ccccc65)cc4)c4cccc(-c5ccc6c(c5)-c5cccc7cccc(c57)O6)c4)cccc32)cc1 ; c1ccc(C2(c3ccccc3)c3ccccc3-c3c(N(c4ccc(-c5ccccc5-n5c6ccccc6c6ccccc65)cc4)c4cccc(-c5ccc6c(c5)-c5cccc7cccc(c57)S6)c4)cccc32)cc1]

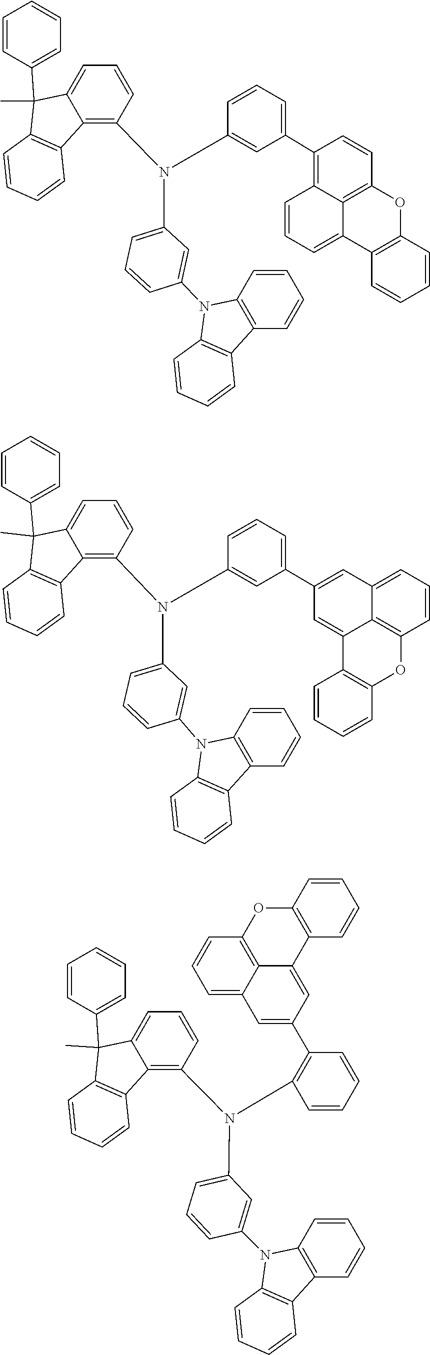 [SMILES: CC1(c2ccccc2)c2ccccc2-c2c(N(c3cccc(-c4ccc5c6c(cccc46)-c4ccccc4O5)c3)c3cccc(-n4c5ccccc5c5ccccc54)c3)cccc21 ; CC1(c2ccccc2)c2ccccc2-c2c(N(c3cccc(-c4cc5c6c(cccc6c4)Oc4ccccc4-5)c3)c3cccc(-n4c5ccccc5c5ccccc54)c3)cccc21 ; CC1(c2ccccc2)c2ccccc2-c2c(N(c3cccc(-n4c5ccccc5c5ccccc54)c3)c3ccccc3-c3cc4c5c(cccc5c3)Oc3ccccc3-4)cccc21]

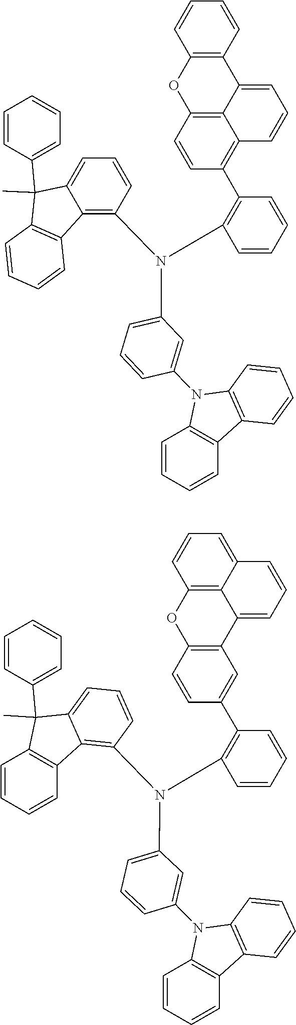

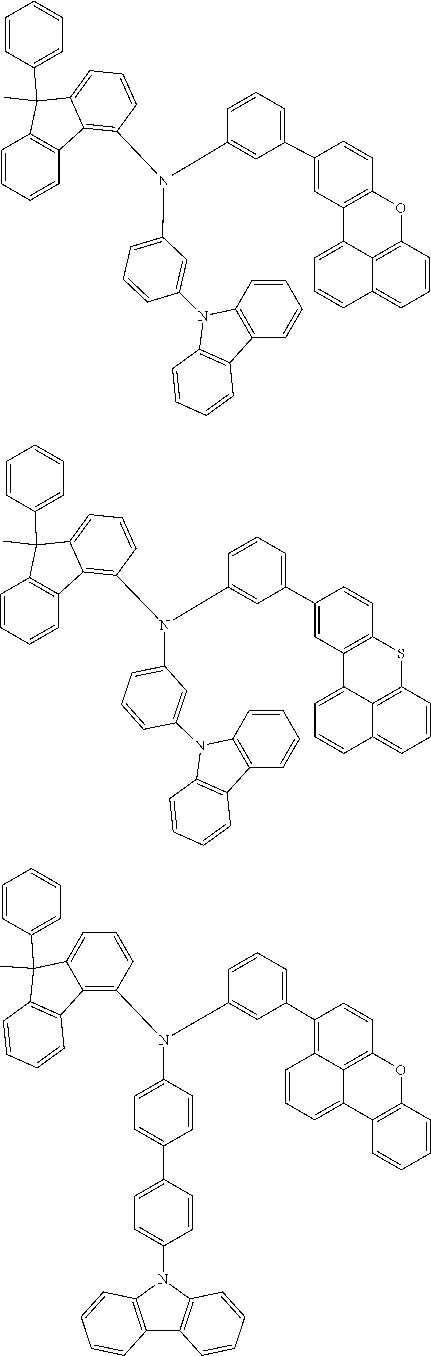 [SMILES: CC1(c2ccccc2)c2ccccc2-c2c(N(c3cccc(-c4ccc5c(c4)-c4cccc6cccc(c46)O5)c3)c3cccc(-n4c5ccccc5c5ccccc54)c3)cccc21 ; CC1(c2ccccc2)c2ccccc2-c2c(N(c3cccc(-c4ccc5c(c4)-c4cccc6cccc(c46)S5)c3)c3cccc(-n4c5ccccc5c5ccccc54)c3)cccc21 ; CC1(c2ccccc2)c2ccccc2-c2c(N(c3ccc(-c4ccc(-n5c6ccccc6c6ccccc65)cc4)cc3)c3cccc(-c4ccc5c6c(cccc46)-c4ccccc4O5)c3)cccc21]

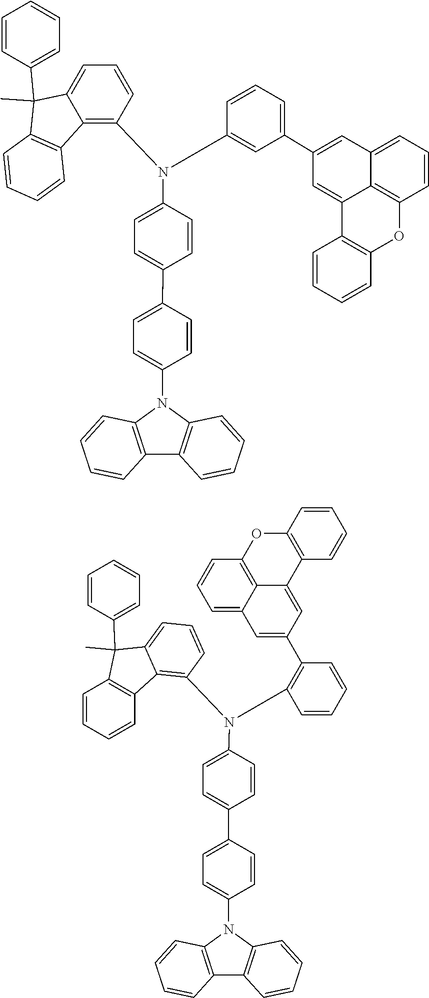 [SMILES: CC1(c2ccccc2)c2ccccc2-c2c(N(c3ccc(-c4ccc(-n5c6ccccc6c6ccccc65)cc4)cc3)c3cccc(-c4cc5c6c(cccc6c4)Oc4ccccc4-5)c3)cccc21 ; CC1(c2ccccc2)c2ccccc2-c2c(N(c3ccc(-c4ccc(-n5c6ccccc6c6ccccc65)cc4)cc3)c3ccccc3-c3cc4c5c(cccc5c3)Oc3ccccc3-4)cccc21]

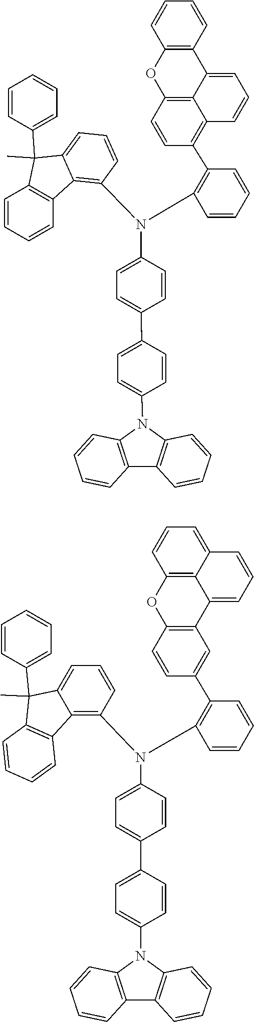 [SMILES: CC1(c2ccccc2)c2ccccc2-c2c(N(c3ccc(-c4ccc(-n5c6ccccc6c6ccccc65)cc4)cc3)c3ccccc3-c3ccc4c5c(cccc35)-c3ccccc3O4)cccc21 ; CC1(c2ccccc2)c2ccccc2-c2c(N(c3ccc(-c4ccc(-n5c6ccccc6c6ccccc65)cc4)cc3)c3ccccc3-c3ccc4c(c3)-c3cccc5cccc(c35)O4)cccc21]

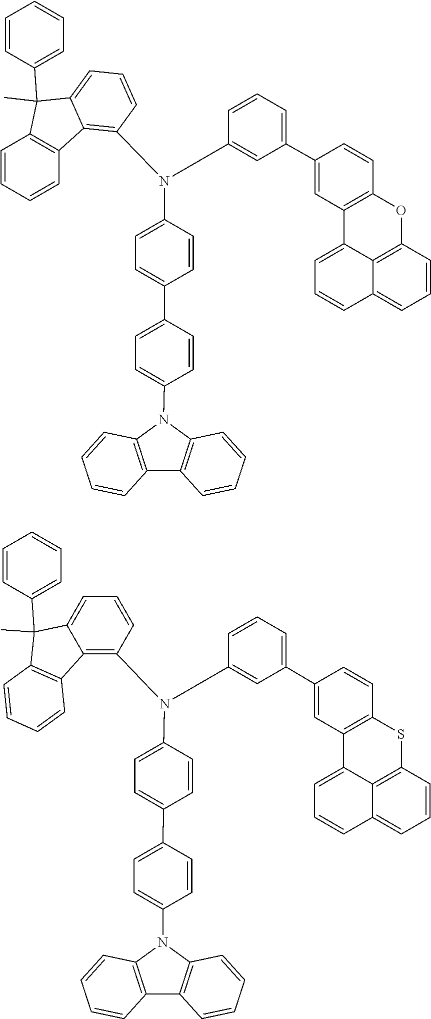 [SMILES: CC1(c2ccccc2)c2ccccc2-c2c(N(c3ccc(-c4ccc(-n5c6ccccc6c6ccccc65)cc4)cc3)c3cccc(-c4ccc5c(c4)-c4cccc6cccc(c46)O5)c3)cccc21 ; CC1(c2ccccc2)c2ccccc2-c2c(N(c3ccc(-c4ccc(-n5c6ccccc6c6ccccc65)cc4)cc3)c3cccc(-c4ccc5c(c4)-c4cccc6cccc(c46)S5)c3)cccc21]

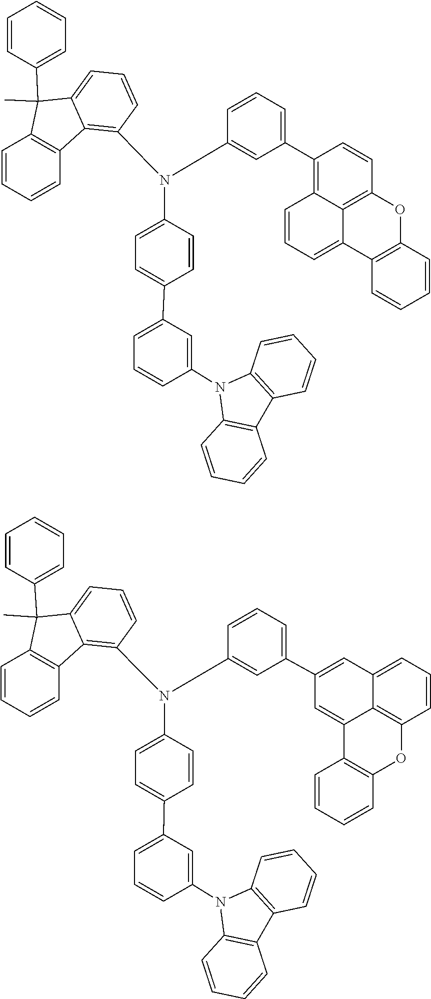 [SMILES: CC1(c2ccccc2)c2ccccc2-c2c(N(c3ccc(-c4cccc(-n5c6ccccc6c6ccccc65)c4)cc3)c3cccc(-c4ccc5c6c(cccc46)-c4ccccc4O5)c3)cccc21 ; CC1(c2ccccc2)c2ccccc2-c2c(N(c3ccc(-c4cccc(-n5c6ccccc6c6ccccc65)c4)cc3)c3cccc(-c4cc5c6c(cccc6c4)Oc4ccccc4-5)c3)cccc21]

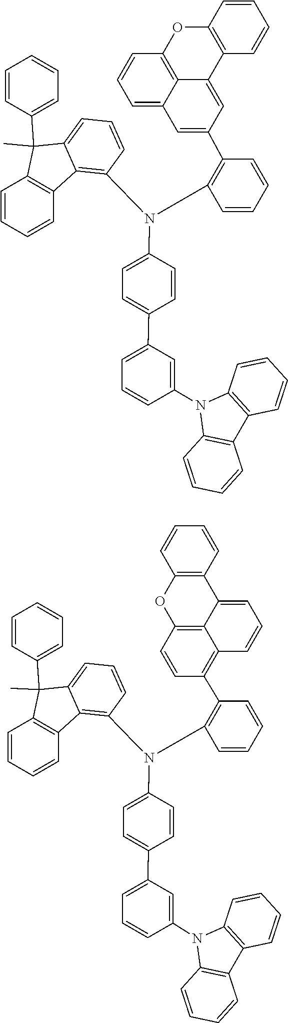

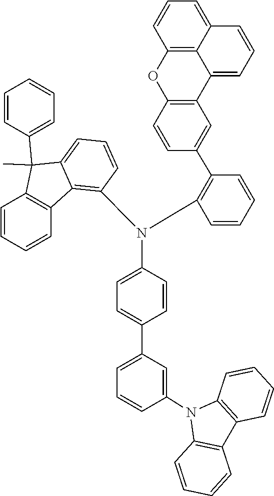 [SMILES: CC1(c2ccccc2)c2ccccc2-c2c(N(c3ccc(-c4cccc(-n5c6ccccc6c6ccccc65)c4)cc3)c3ccccc3-c3ccc4c(c3)-c3cccc5cccc(c35)O4)cccc21]

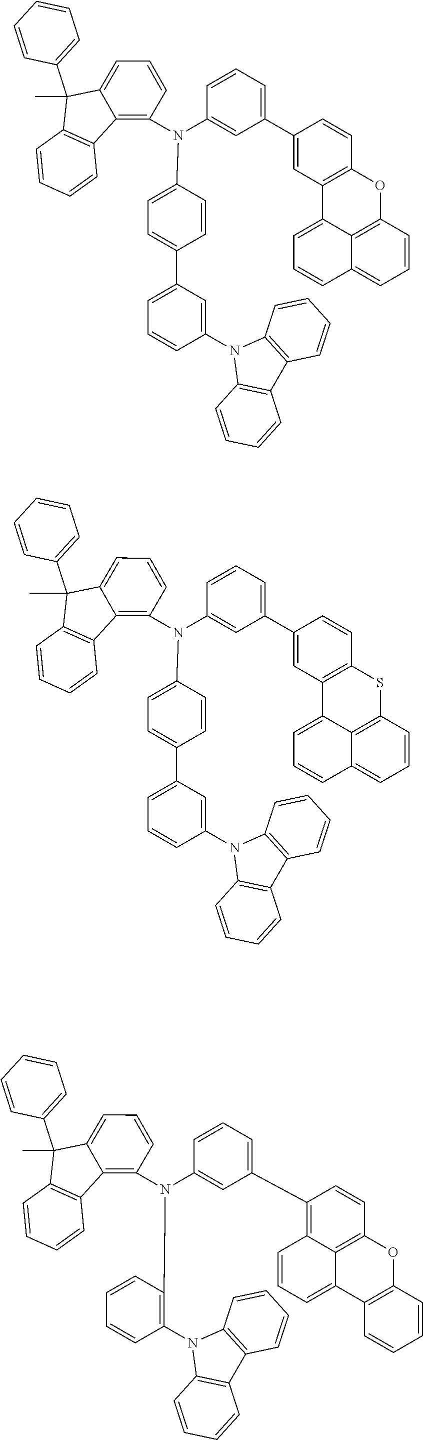 [SMILES: CC1(c2ccccc2)c2ccccc2-c2c(N(c3ccc(-c4cccc(-n5c6ccccc6c6ccccc65)c4)cc3)c3cccc(-c4ccc5c(c4)-c4cccc6cccc(c46)O5)c3)cccc21 ; CC1(c2ccccc2)c2ccccc2-c2c(N(c3ccc(-c4cccc(-n5c6ccccc6c6ccccc65)c4)cc3)c3cccc(-c4ccc5c(c4)-c4cccc6cccc(c46)S5)c3)cccc21 ; CC1(c2ccccc2)c2ccccc2-c2c(N(c3cccc(-c4ccc5c6c(cccc46)-c4ccccc4O5)c3)c3ccccc3-n3c4ccccc4c4ccccc43)cccc21]

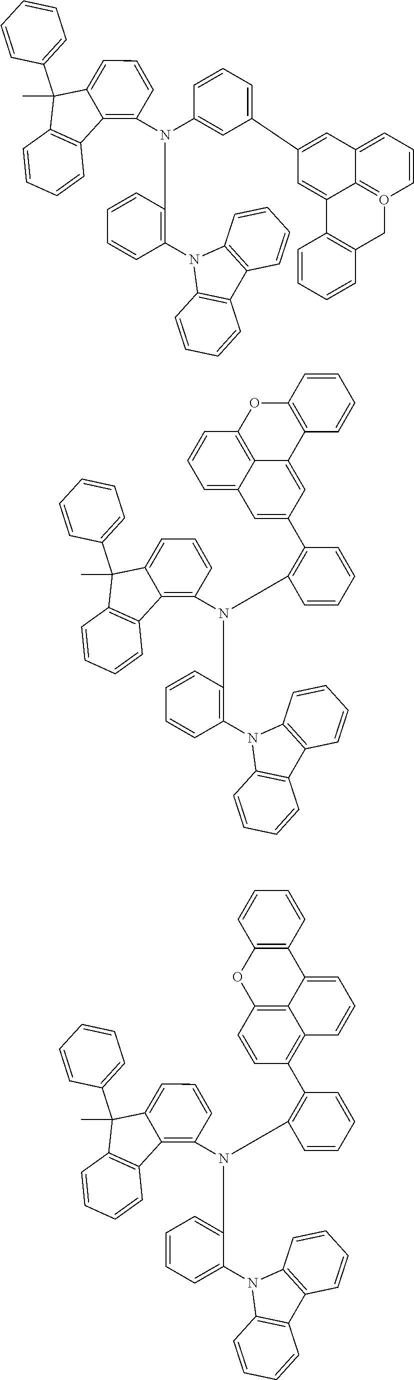

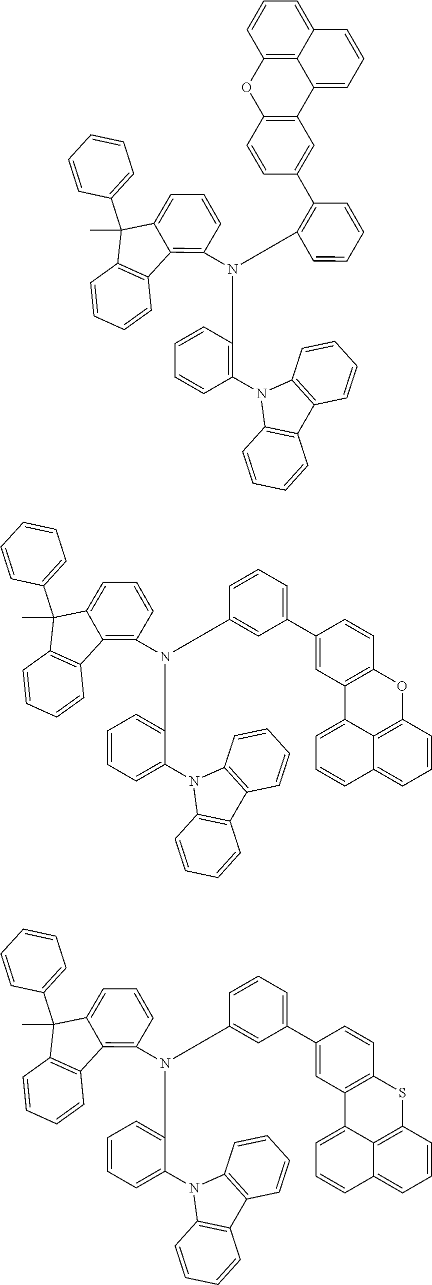 [SMILES: CC1(c2ccccc2)c2ccccc2-c2c(N(c3ccccc3-c3ccc4c(c3)-c3cccc5cccc(c35)O4)c3ccccc3-n3c4ccccc4c4ccccc43)cccc21 ; CC1(c2ccccc2)c2ccccc2-c2c(N(c3cccc(-c4ccc5c(c4)-c4cccc6cccc(c46)O5)c3)c3ccccc3-n3c4ccccc4c4ccccc43)cccc21 ; CC1(c2ccccc2)c2ccccc2-c2c(N(c3cccc(-c4ccc5c(c4)-c4cccc6cccc(c46)S5)c3)c3ccccc3-n3c4ccccc4c4ccccc43)cccc21]

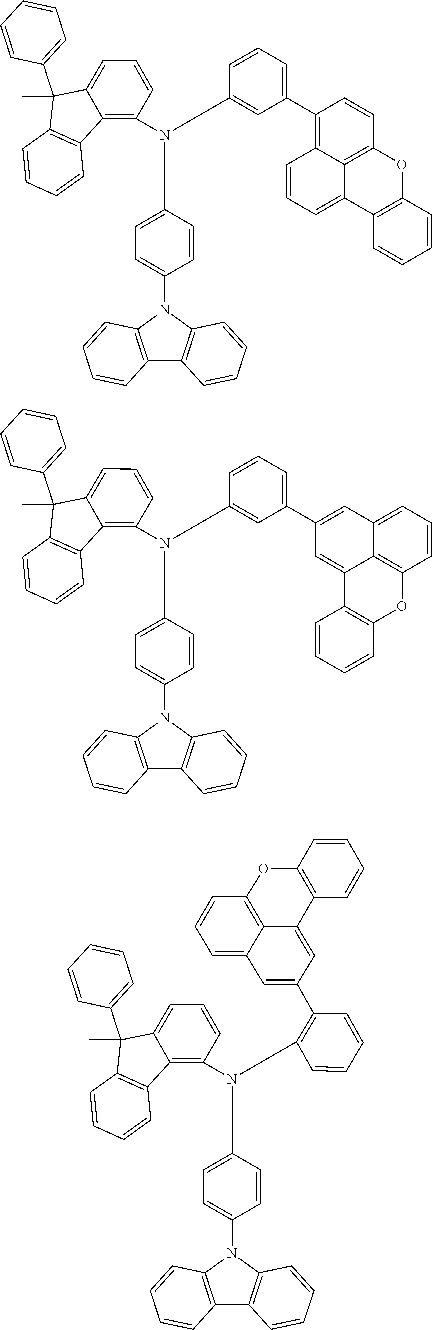 [SMILES: CC1(c2ccccc2)c2ccccc2-c2c(N(c3ccc(-n4c5ccccc5c5ccccc54)cc3)c3cccc(-c4ccc5c6c(cccc46)-c4ccccc4O5)c3)cccc21 ; CC1(c2ccccc2)c2ccccc2-c2c(N(c3ccc(-n4c5ccccc5c5ccccc54)cc3)c3cccc(-c4cc5c6c(cccc6c4)Oc4ccccc4-5)c3)cccc21 ; CC1(c2ccccc2)c2ccccc2-c2c(N(c3ccc(-n4c5ccccc5c5ccccc54)cc3)c3ccccc3-c3cc4c5c(cccc5c3)Oc3ccccc3-4)cccc21]

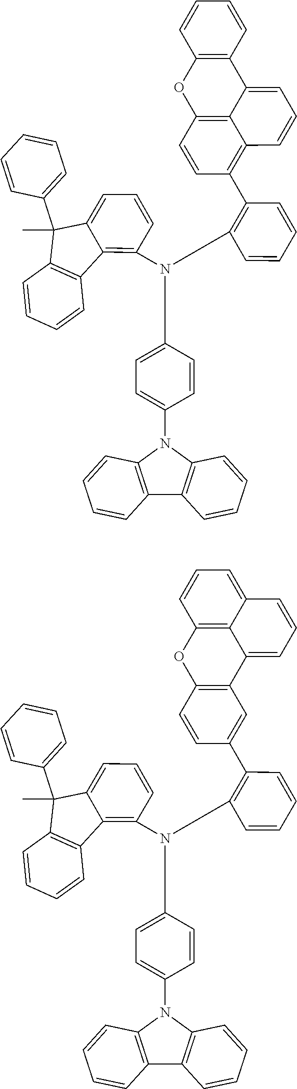 [SMILES: CC1(c2ccccc2)c2ccccc2-c2c(N(c3ccc(-n4c5ccccc5c5ccccc54)cc3)c3ccccc3-c3ccc4c5c(cccc35)-c3ccccc3O4)cccc21 ; CC1(c2ccccc2)c2ccccc2-c2c(N(c3ccc(-n4c5ccccc5c5ccccc54)cc3)c3ccccc3-c3ccc4c(c3)-c3cccc5cccc(c35)O4)cccc21]

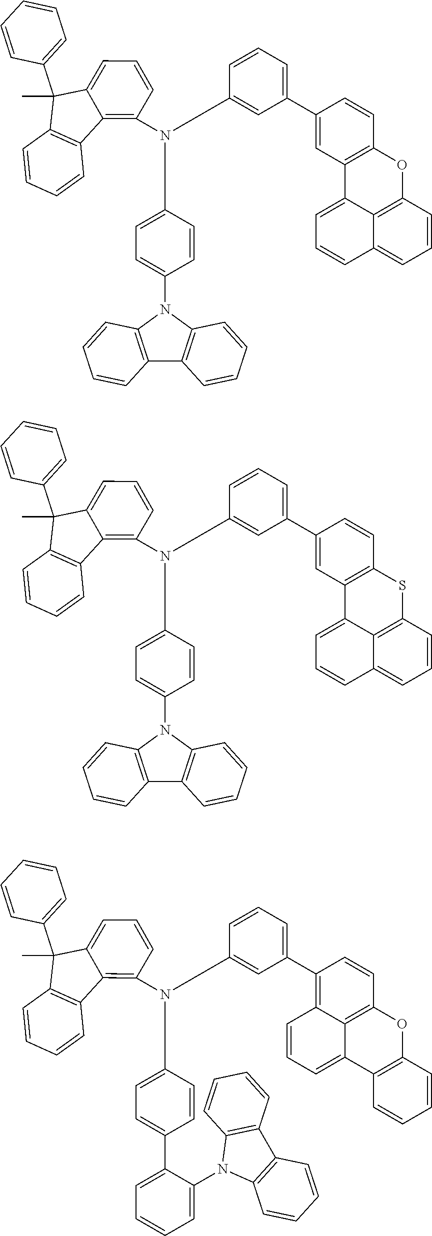 [SMILES: CC1(c2ccccc2)c2ccccc2-c2c(N(c3ccc(-n4c5ccccc5c5ccccc54)cc3)c3cccc(-c4ccc5c(c4)-c4cccc6cccc(c46)O5)c3)cccc21 ; CC1(c2ccccc2)c2ccccc2-c2c(N(c3ccc(-n4c5ccccc5c5ccccc54)cc3)c3cccc(-c4ccc5c(c4)-c4cccc6cccc(c46)S5)c3)cccc21 ; CC1(c2ccccc2)c2ccccc2-c2c(N(c3ccc(-c4ccccc4-n4c5ccccc5c5ccccc54)cc3)c3cccc(-c4ccc5c6c(cccc46)-c4ccccc4O5)c3)cccc21]

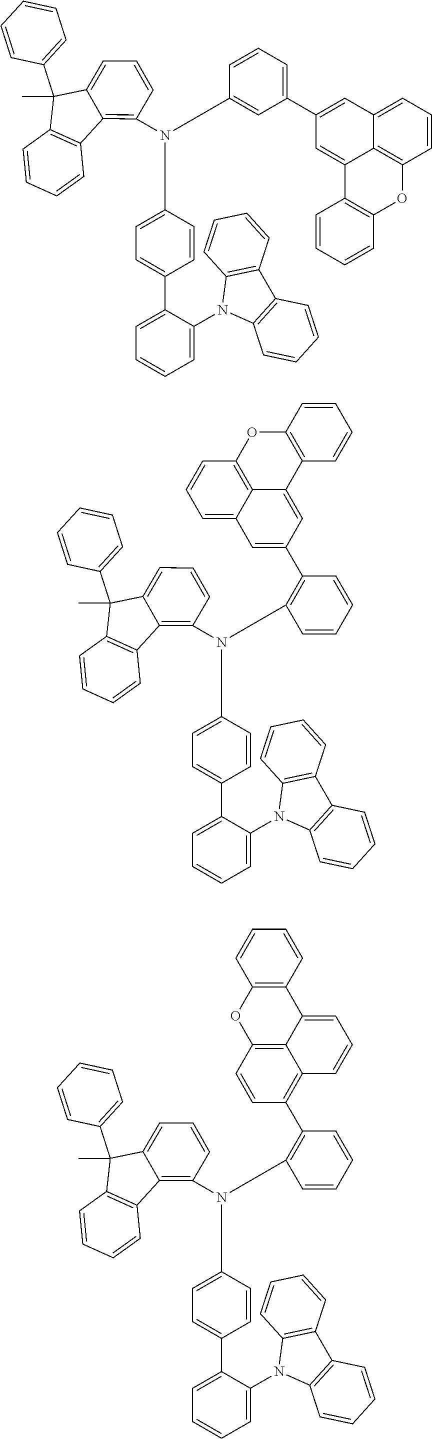

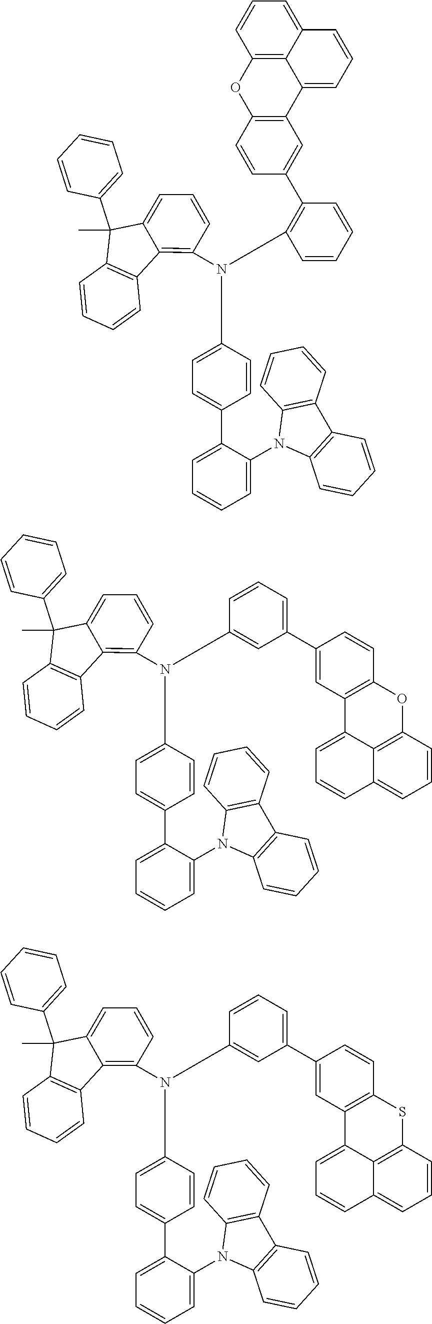 [SMILES: CC1(c2ccccc2)c2ccccc2-c2c(N(c3ccc(-c4ccccc4-n4c5ccccc5c5ccccc54)cc3)c3ccccc3-c3ccc4c(c3)-c3cccc5cccc(c35)O4)cccc21 ; CC1(c2ccccc2)c2ccccc2-c2c(N(c3ccc(-c4ccccc4-n4c5ccccc5c5ccccc54)cc3)c3cccc(-c4ccc5c(c4)-c4cccc6cccc(c46)O5)c3)cccc21 ; CC1(c2ccccc2)c2ccccc2-c2c(N(c3ccc(-c4ccccc4-n4c5ccccc5c5ccccc54)cc3)c3cccc(-c4ccc5c(c4)-c4cccc6cccc(c46)S5)c3)cccc21]

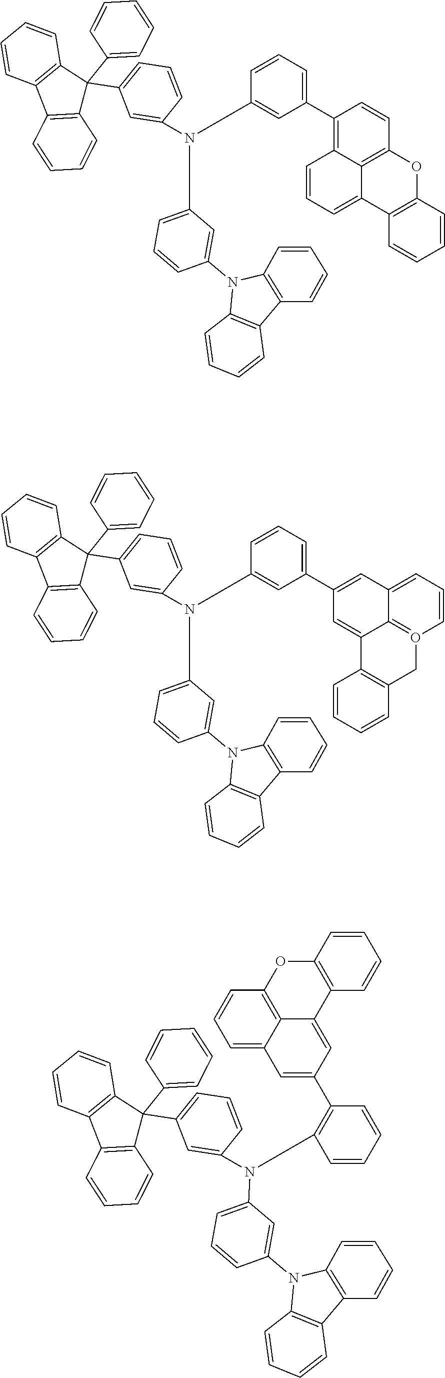

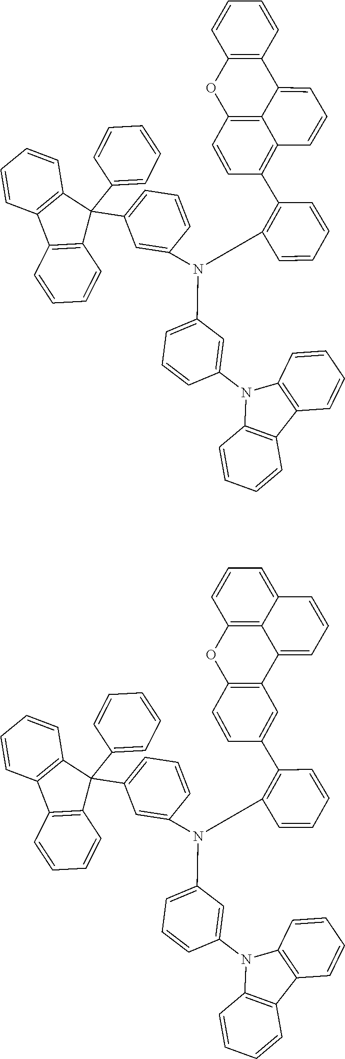 [SMILES: c1ccc(C2(c3cccc(N(c4cccc(-n5c6ccccc6c6ccccc65)c4)c4ccccc4-c4ccc5c6c(cccc46)-c4ccccc4O5)c3)c3ccccc3-c3ccccc32)cc1 ; c1ccc(C2(c3cccc(N(c4cccc(-n5c6ccccc6c6ccccc65)c4)c4ccccc4-c4ccc5c(c4)-c4cccc6cccc(c46)O5)c3)c3ccccc3-c3ccccc32)cc1]

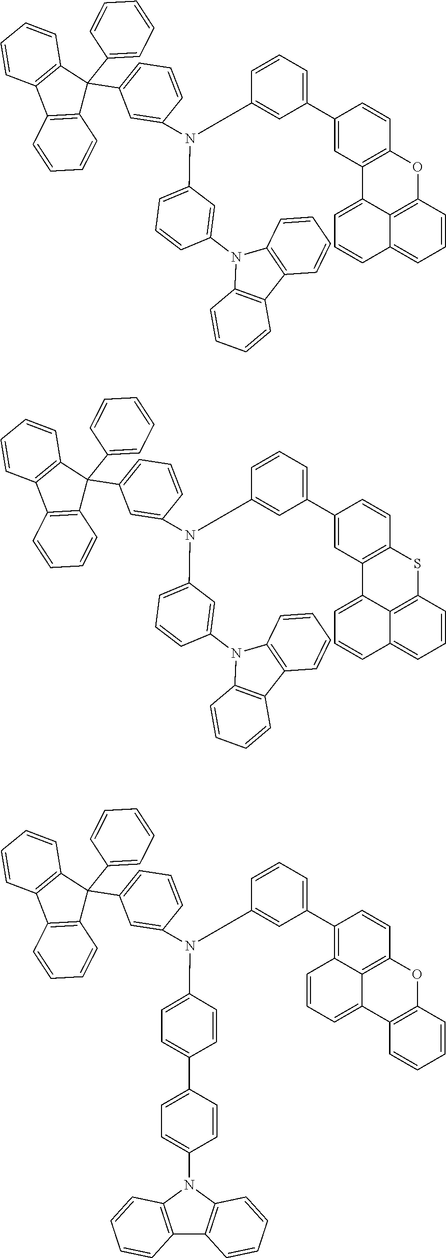 [SMILES: c1ccc(C2(c3cccc(N(c4cccc(-c5ccc6c(c5)-c5cccc7cccc(c57)O6)c4)c4cccc(-n5c6ccccc6c6ccccc65)c4)c3)c3ccccc3-c3ccccc32)cc1 ; c1ccc(C2(c3cccc(N(c4cccc(-c5ccc6c(c5)-c5cccc7cccc(c57)S6)c4)c4cccc(-n5c6ccccc6c6ccccc65)c4)c3)c3ccccc3-c3ccccc32)cc1 ; c1ccc(C2(c3cccc(N(c4ccc(-c5ccc(-n6c7ccccc7c7ccccc76)cc5)cc4)c4cccc(-c5ccc6c7c(cccc57)-c5ccccc5O6)c4)c3)c3ccccc3-c3ccccc32)cc1]

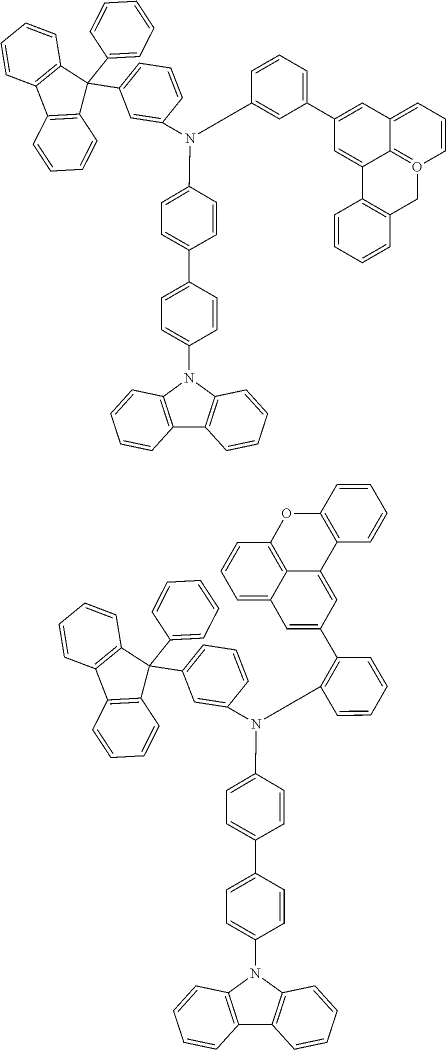

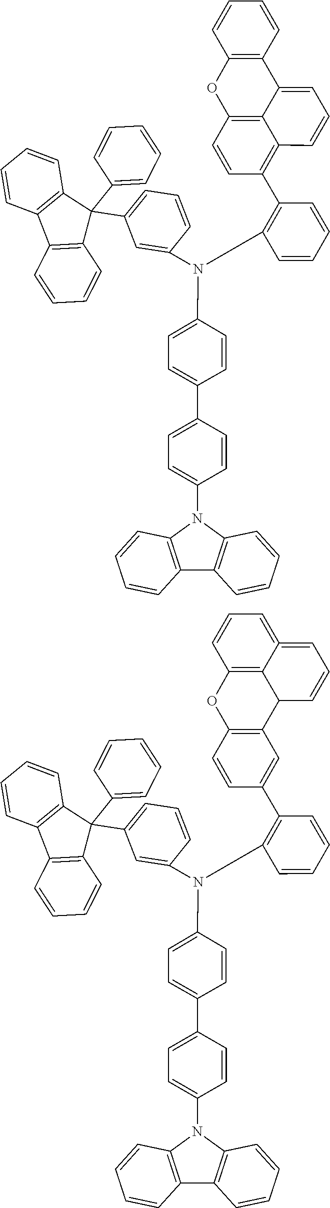

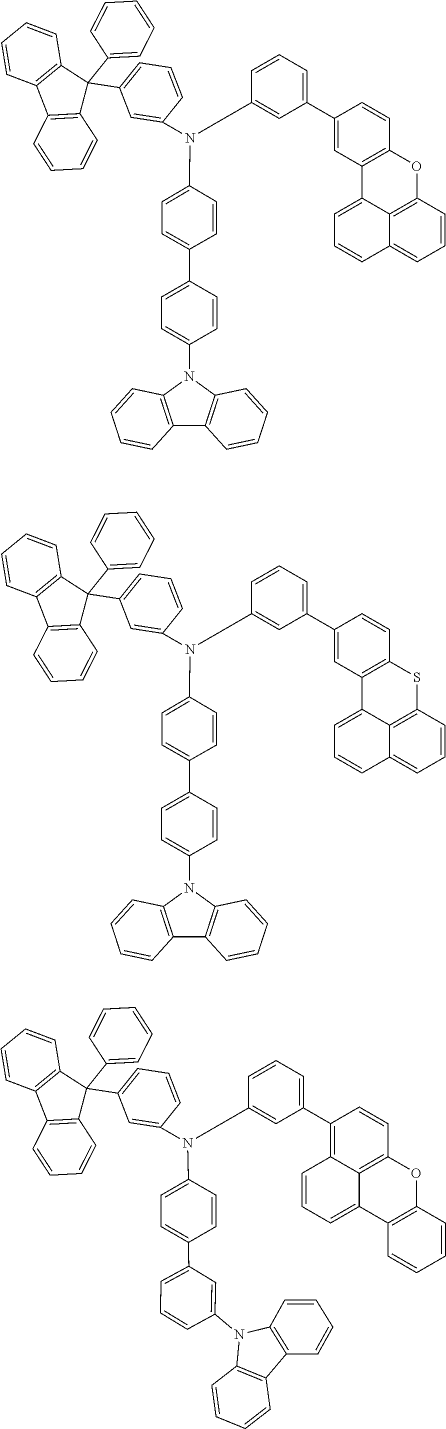 [SMILES: c1ccc(C2(c3cccc(N(c4ccc(-c5ccc(-n6c7ccccc7c7ccccc76)cc5)cc4)c4cccc(-c5ccc6c(c5)-c5cccc7cccc(c57)O6)c4)c3)c3ccccc3-c3ccccc32)cc1 ; c1ccc(C2(c3cccc(N(c4ccc(-c5ccc(-n6c7ccccc7c7ccccc76)cc5)cc4)c4cccc(-c5ccc6c(c5)-c5cccc7cccc(c57)S6)c4)c3)c3ccccc3-c3ccccc32)cc1 ; c1ccc(C2(c3cccc(N(c4ccc(-c5cccc(-n6c7ccccc7c7ccccc76)c5)cc4)c4cccc(-c5ccc6c7c(cccc57)-c5ccccc5O6)c4)c3)c3ccccc3-c3ccccc32)cc1]

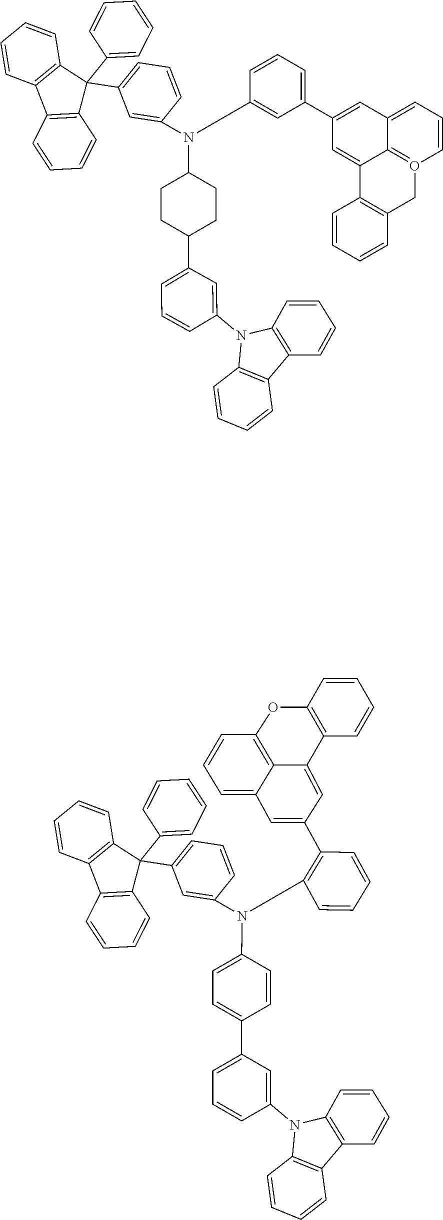

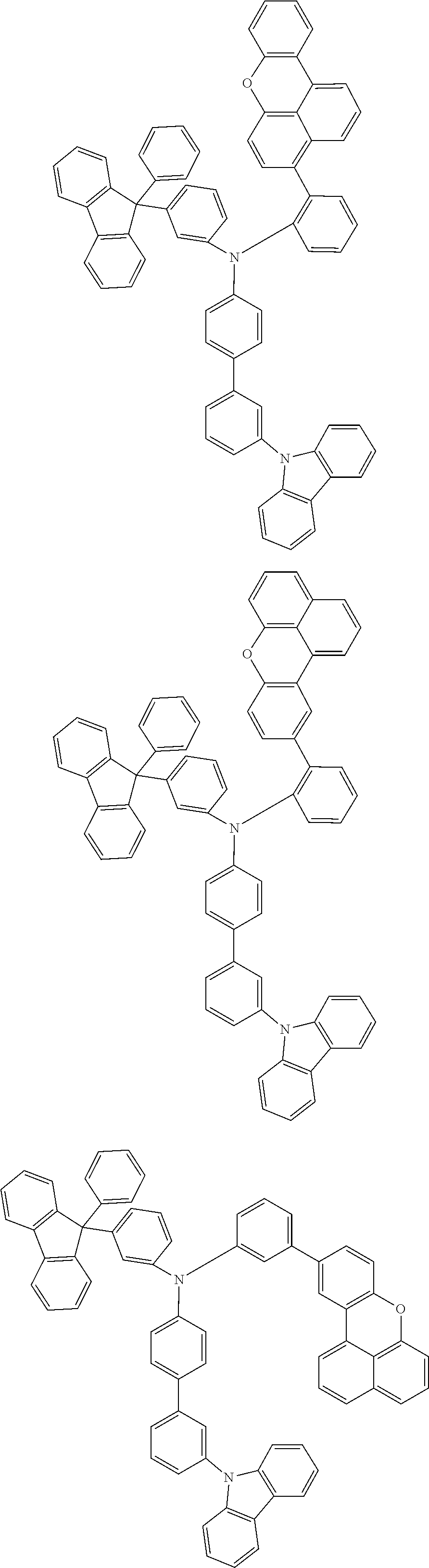

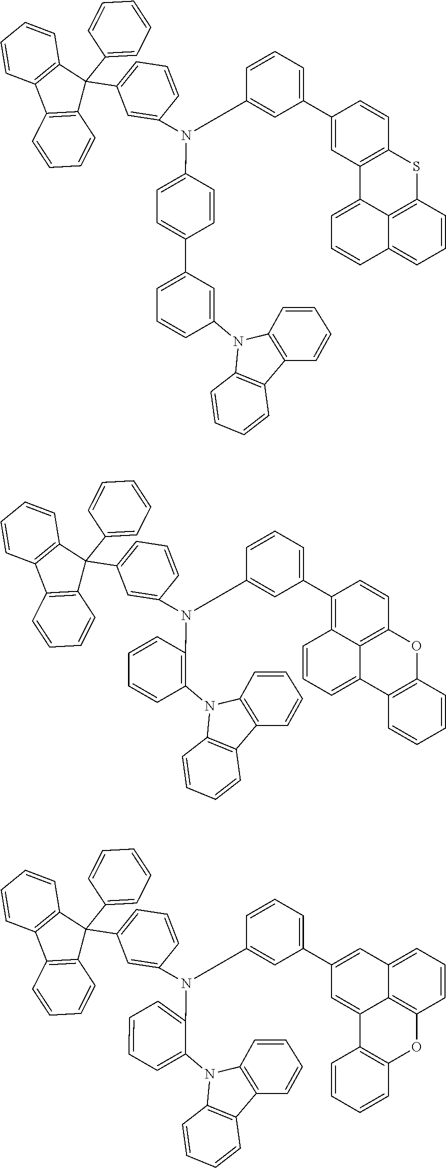 [SMILES: c1ccc(C2(c3cccc(N(c4ccc(-c5cccc(-n6c7ccccc7c7ccccc76)c5)cc4)c4cccc(-c5ccc6c(c5)-c5cccc7cccc(c57)S6)c4)c3)c3ccccc3-c3ccccc32)cc1 ; c1ccc(C2(c3cccc(N(c4cccc(-c5ccc6c7c(cccc57)-c5ccccc5O6)c4)c4ccccc4-n4c5ccccc5c5ccccc54)c3)c3ccccc3-c3ccccc32)cc1 ; c1ccc(C2(c3cccc(N(c4cccc(-c5cc6c7c(cccc7c5)Oc5ccccc5-6)c4)c4ccccc4-n4c5ccccc5c5ccccc54)c3)c3ccccc3-c3ccccc32)cc1]

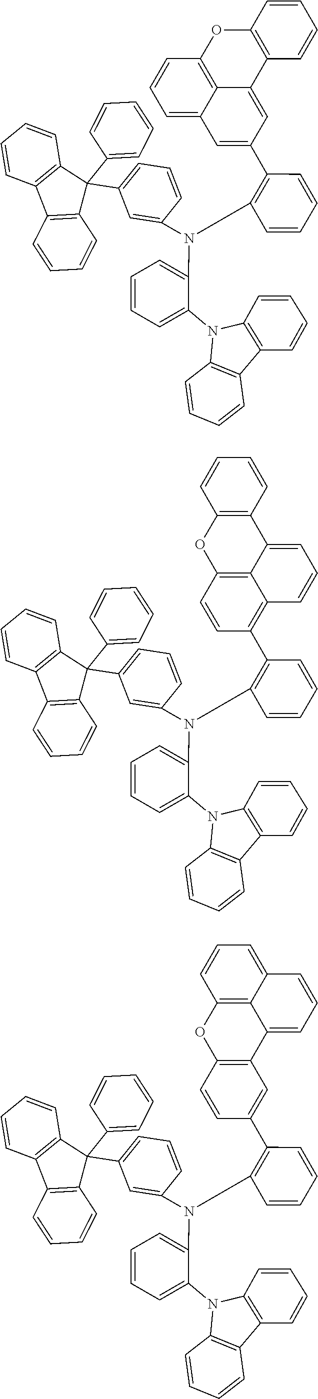

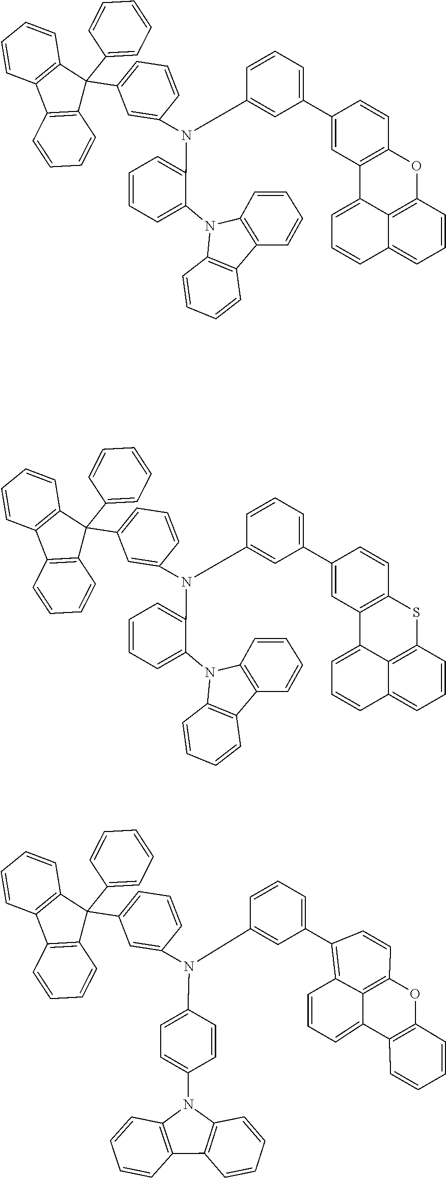 [SMILES: c1ccc(C2(c3cccc(N(c4cccc(-c5ccc6c(c5)-c5cccc7cccc(c57)O6)c4)c4ccccc4-n4c5ccccc5c5ccccc54)c3)c3ccccc3-c3ccccc32)cc1 ; c1ccc(C2(c3cccc(N(c4cccc(-c5ccc6c(c5)-c5cccc7cccc(c57)S6)c4)c4ccccc4-n4c5ccccc5c5ccccc54)c3)c3ccccc3-c3ccccc32)cc1 ; c1ccc(C2(c3cccc(N(c4ccc(-n5c6ccccc6c6ccccc65)cc4)c4cccc(-c5ccc6c7c(cccc57)-c5ccccc5O6)c4)c3)c3ccccc3-c3ccccc32)cc1]

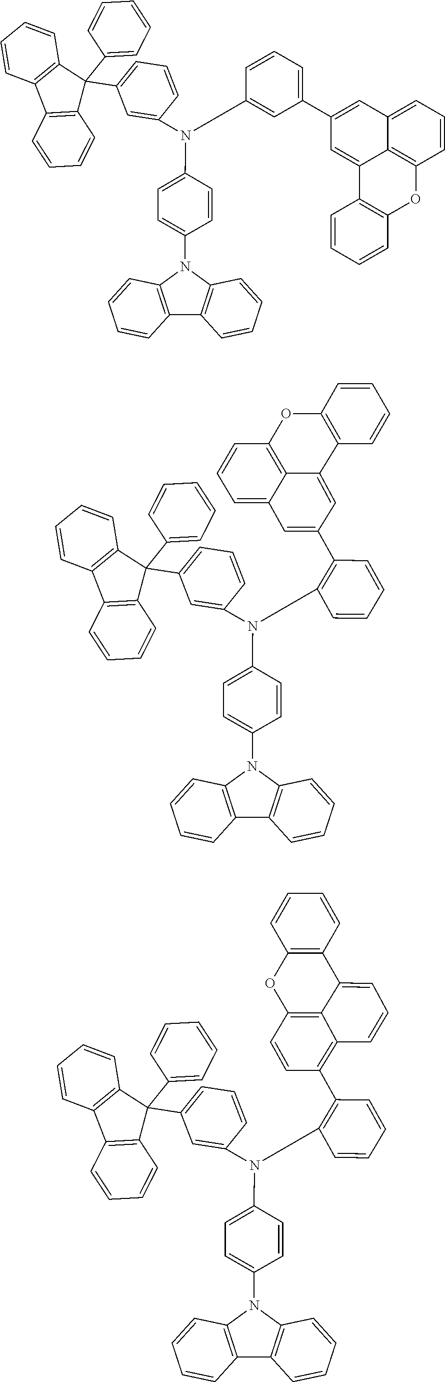 [SMILES: c1ccc(C2(c3cccc(N(c4ccc(-n5c6ccccc6c6ccccc65)cc4)c4cccc(-c5cc6c7c(cccc7c5)Oc5ccccc5-6)c4)c3)c3ccccc3-c3ccccc32)cc1 ; c1ccc(C2(c3cccc(N(c4ccc(-n5c6ccccc6c6ccccc65)cc4)c4ccccc4-c4cc5c6c(cccc6c4)Oc4ccccc4-5)c3)c3ccccc3-c3ccccc32)cc1 ; c1ccc(C2(c3cccc(N(c4ccc(-n5c6ccccc6c6ccccc65)cc4)c4ccccc4-c4ccc5c6c(cccc46)-c4ccccc4O5)c3)c3ccccc3-c3ccccc32)cc1]

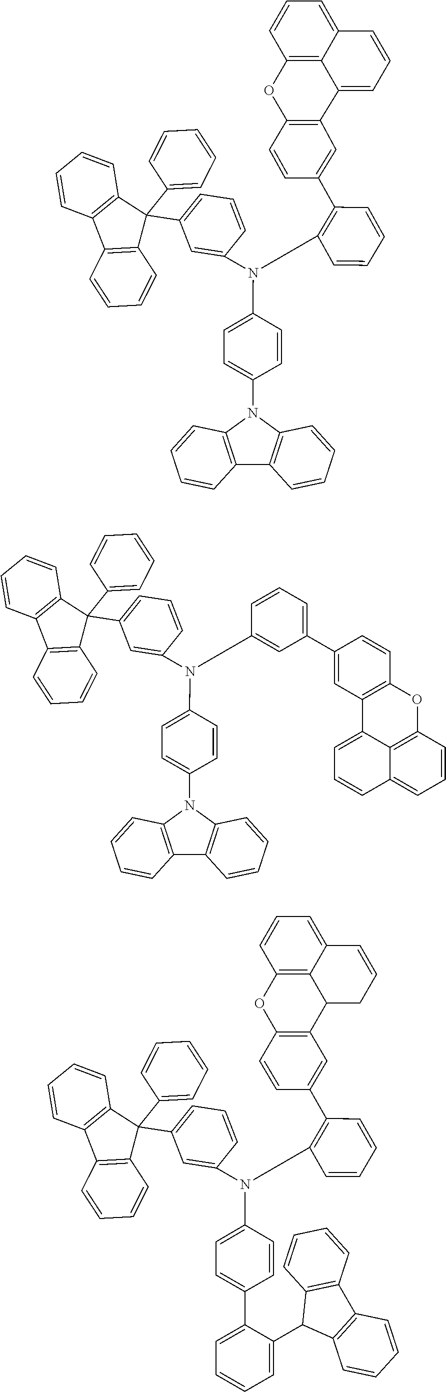 [SMILES: c1ccc(C2(c3cccc(N(c4ccc(-n5c6ccccc6c6ccccc65)cc4)c4ccccc4-c4ccc5c(c4)-c4cccc6cccc(c46)O5)c3)c3ccccc3-c3ccccc32)cc1 ; c1ccc(C2(c3cccc(N(c4ccc(-n5c6ccccc6c6ccccc65)cc4)c4cccc(-c5ccc6c(c5)-c5cccc7cccc(c57)O6)c4)c3)c3ccccc3-c3ccccc32)cc1 ; C1=Cc2cccc3c2C(C1)c1cc(-c2ccccc2N(c2ccc(-c4ccccc4C4c5ccccc5-c5ccccc54)cc2)c2cccc(C4(c5ccccc5)c5ccccc5-c5ccccc54)c2)ccc1O3]

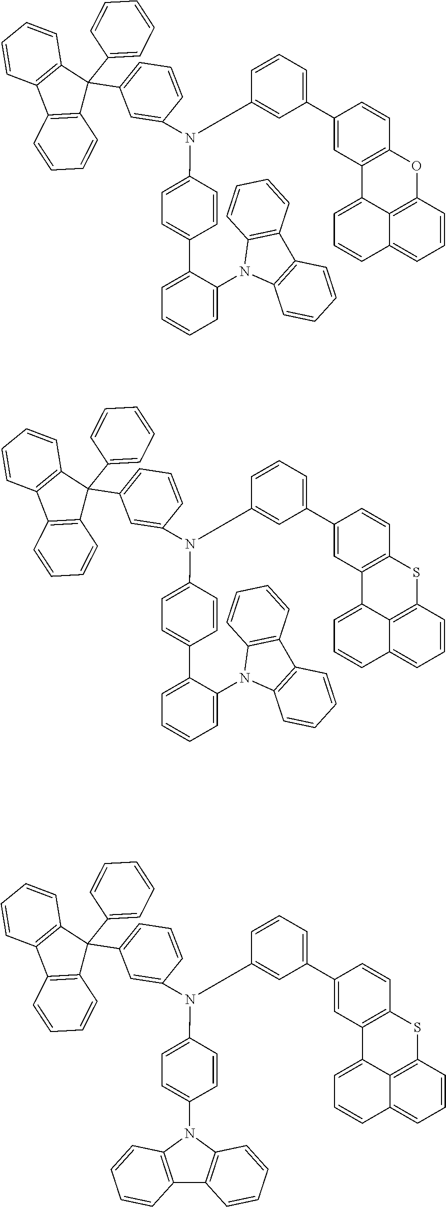 [SMILES: c1ccc(C2(c3cccc(N(c4ccc(-c5ccccc5-n5c6ccccc6c6ccccc65)cc4)c4cccc(-c5ccc6c(c5)-c5cccc7cccc(c57)O6)c4)c3)c3ccccc3-c3ccccc32)cc1 ; c1ccc(C2(c3cccc(N(c4ccc(-c5ccccc5-n5c6ccccc6c6ccccc65)cc4)c4cccc(-c5ccc6c(c5)-c5cccc7cccc(c57)S6)c4)c3)c3ccccc3-c3ccccc32)cc1 ; c1ccc(C2(c3cccc(N(c4ccc(-n5c6ccccc6c6ccccc65)cc4)c4cccc(-c5ccc6c(c5)-c5cccc7cccc(c57)S6)c4)c3)c3ccccc3-c3ccccc32)cc1]

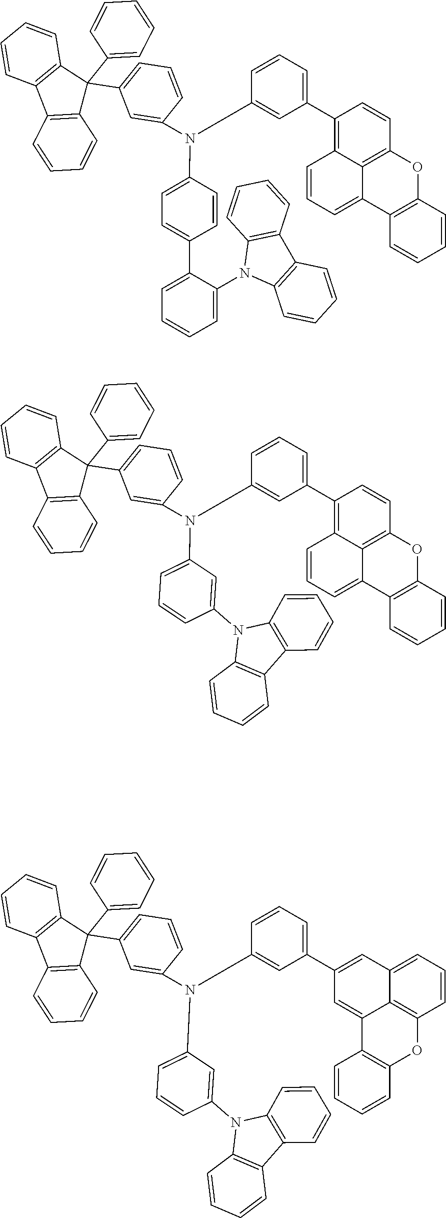 [SMILES: c1ccc(C2(c3cccc(N(c4ccc(-c5ccccc5-n5c6ccccc6c6ccccc65)cc4)c4cccc(-c5ccc6c7c(cccc57)-c5ccccc5O6)c4)c3)c3ccccc3-c3ccccc32)cc1 ; c1ccc(C2(c3cccc(N(c4cccc(-c5ccc6c7c(cccc57)-c5ccccc5O6)c4)c4cccc(-n5c6ccccc6c6ccccc65)c4)c3)c3ccccc3-c3ccccc32)cc1 ; c1ccc(C2(c3cccc(N(c4cccc(-c5cc6c7c(cccc7c5)Oc5ccccc5-6)c4)c4cccc(-n5c6ccccc6c6ccccc65)c4)c3)c3ccccc3-c3ccccc32)cc1]

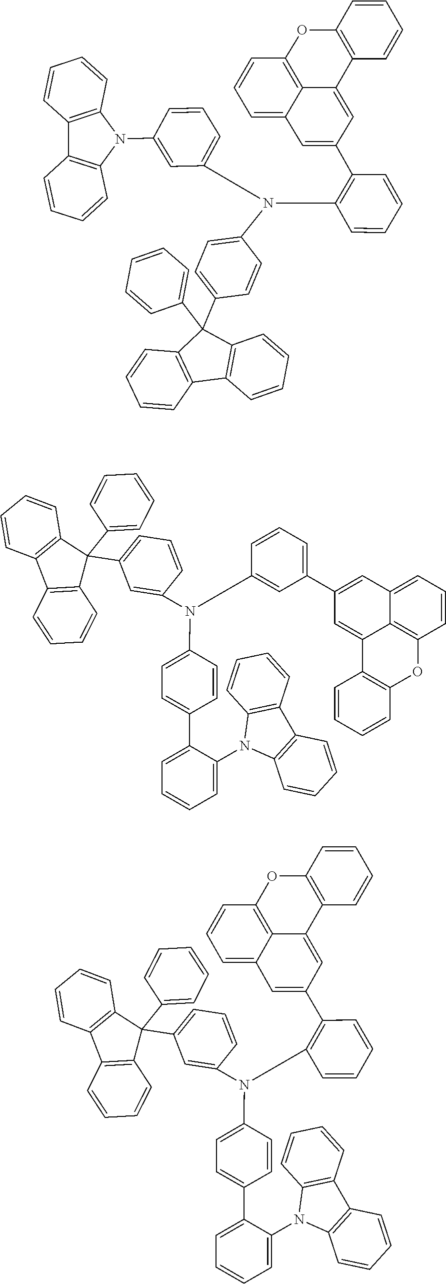 [SMILES: c1ccc(C2(c3ccc(N(c4cccc(-n5c6ccccc6c6ccccc65)c4)c4ccccc4-c4cc5c6c(cccc6c4)Oc4ccccc4-5)cc3)c3ccccc3-c3ccccc32)cc1 ; c1ccc(C2(c3cccc(N(c4ccc(-c5ccccc5-n5c6ccccc6c6ccccc65)cc4)c4cccc(-c5cc6c7c(cccc7c5)Oc5ccccc5-6)c4)c3)c3ccccc3-c3ccccc32)cc1 ; c1ccc(C2(c3cccc(N(c4ccc(-c5ccccc5-n5c6ccccc6c6ccccc65)cc4)c4ccccc4-c4cc5c6c(cccc6c4)Oc4ccccc4-5)c3)c3ccccc3-c3ccccc32)cc1]

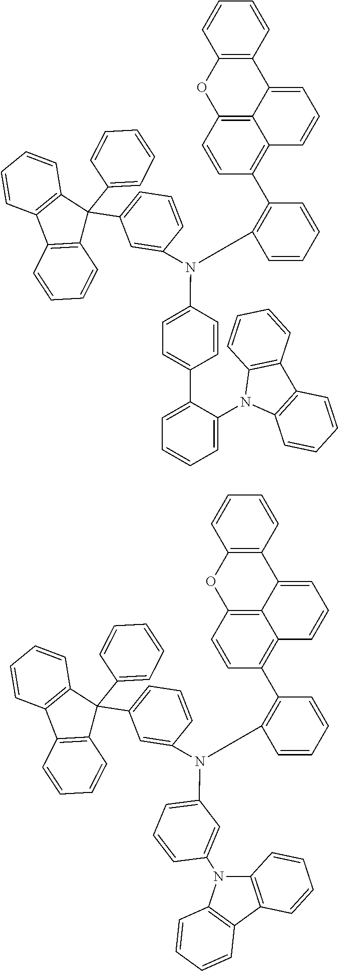 [SMILES: c1ccc(C2(c3cccc(N(c4ccc(-c5ccccc5-n5c6ccccc6c6ccccc65)cc4)c4ccccc4-c4ccc5c6c(cccc46)-c4ccccc4O5)c3)c3ccccc3-c3ccccc32)cc1 ; c1ccc(C2(c3cccc(N(c4cccc(-n5c6ccccc6c6ccccc65)c4)c4ccccc4-c4ccc5c6c(cccc46)-c4ccccc4O5)c3)c3ccccc3-c3ccccc32)cc1]

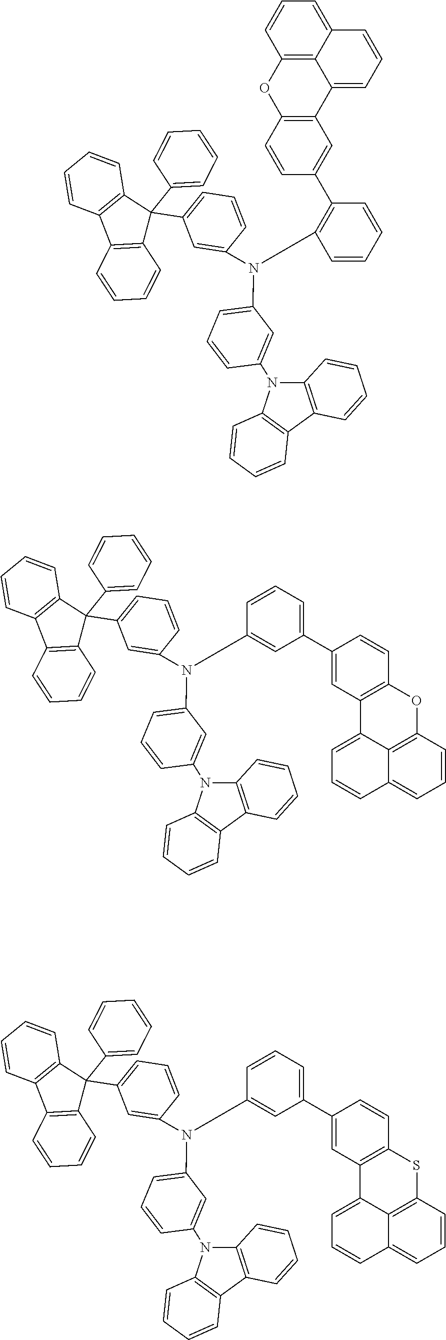 [SMILES: c1ccc(C2(c3cccc(N(c4cccc(-n5c6ccccc6c6ccccc65)c4)c4ccccc4-c4ccc5c(c4)-c4cccc6cccc(c46)O5)c3)c3ccccc3-c3ccccc32)cc1 ; c1ccc(C2(c3cccc(N(c4cccc(-c5ccc6c(c5)-c5cccc7cccc(c57)O6)c4)c4cccc(-n5c6ccccc6c6ccccc65)c4)c3)c3ccccc3-c3ccccc32)cc1 ; c1ccc(C2(c3cccc(N(c4cccc(-c5ccc6c(c5)-c5cccc7cccc(c57)S6)c4)c4cccc(-n5c6ccccc6c6ccccc65)c4)c3)c3ccccc3-c3ccccc32)cc1]

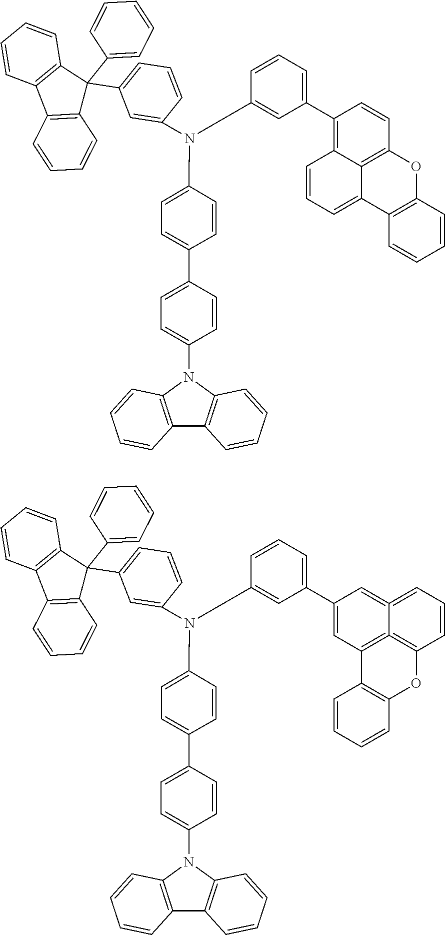

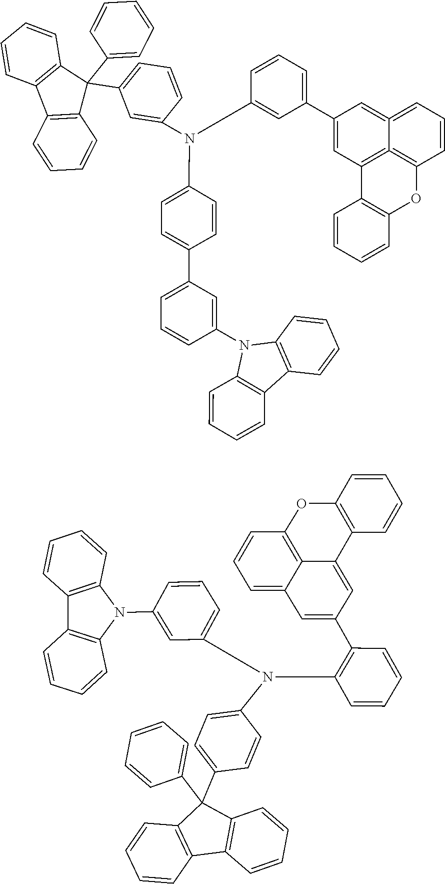

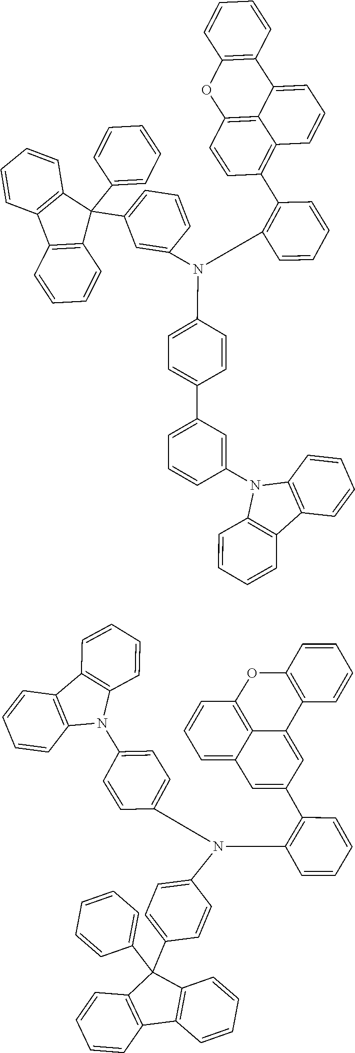 [SMILES: c1ccc(C2(c3cccc(N(c4ccc(-c5cccc(-n6c7ccccc7c7ccccc76)c5)cc4)c4ccccc4-c4ccc5c6c(cccc46)-c4ccccc4O5)c3)c3ccccc3-c3ccccc32)cc1 ; c1ccc(C2(c3ccc(N(c4ccc(-n5c6ccccc6c6ccccc65)cc4)c4ccccc4-c4cc5c6c(cccc6c4)Oc4ccccc4-5)cc3)c3ccccc3-c3ccccc32)cc1]

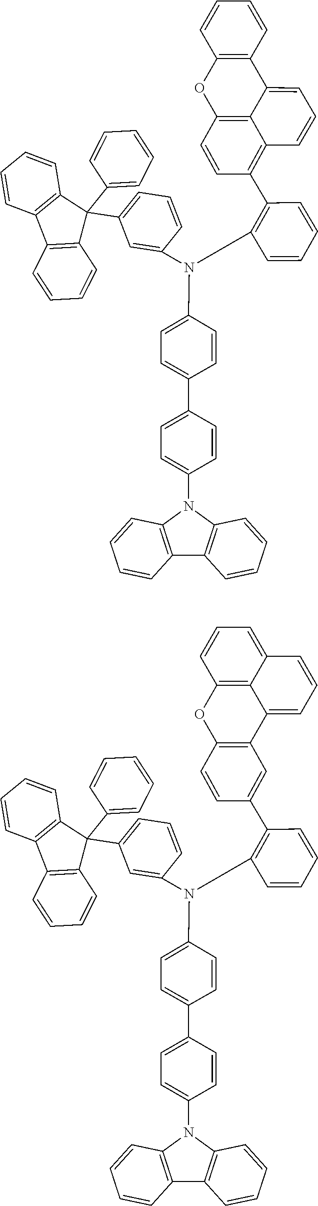 [SMILES: c1ccc(C2(c3cccc(N(c4ccc(-c5ccc(-n6c7ccccc7c7ccccc76)cc5)cc4)c4ccccc4-c4ccc5c6c(cccc46)-c4ccccc4O5)c3)c3ccccc3-c3ccccc32)cc1 ; c1ccc(C2(c3cccc(N(c4ccc(-c5ccc(-n6c7ccccc7c7ccccc76)cc5)cc4)c4ccccc4-c4ccc5c(c4)-c4cccc6cccc(c46)O5)c3)c3ccccc3-c3ccccc32)cc1]

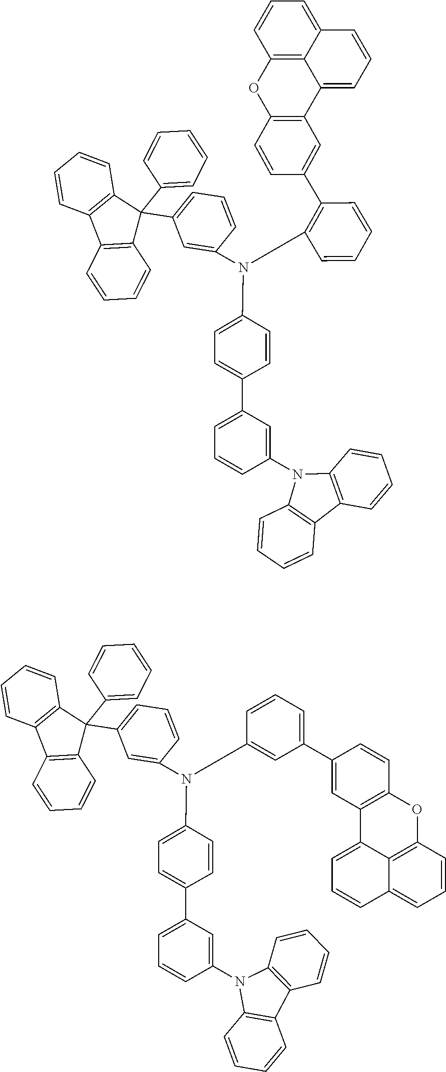 [SMILES: c1ccc(C2(c3cccc(N(c4ccc(-c5cccc(-n6c7ccccc7c7ccccc76)c5)cc4)c4ccccc4-c4ccc5c(c4)-c4cccc6cccc(c46)O5)c3)c3ccccc3-c3ccccc32)cc1 ; c1ccc(C2(c3cccc(N(c4ccc(-c5cccc(-n6c7ccccc7c7ccccc76)c5)cc4)c4cccc(-c5ccc6c(c5)-c5cccc7cccc(c57)O6)c4)c3)c3ccccc3-c3ccccc32)cc1]

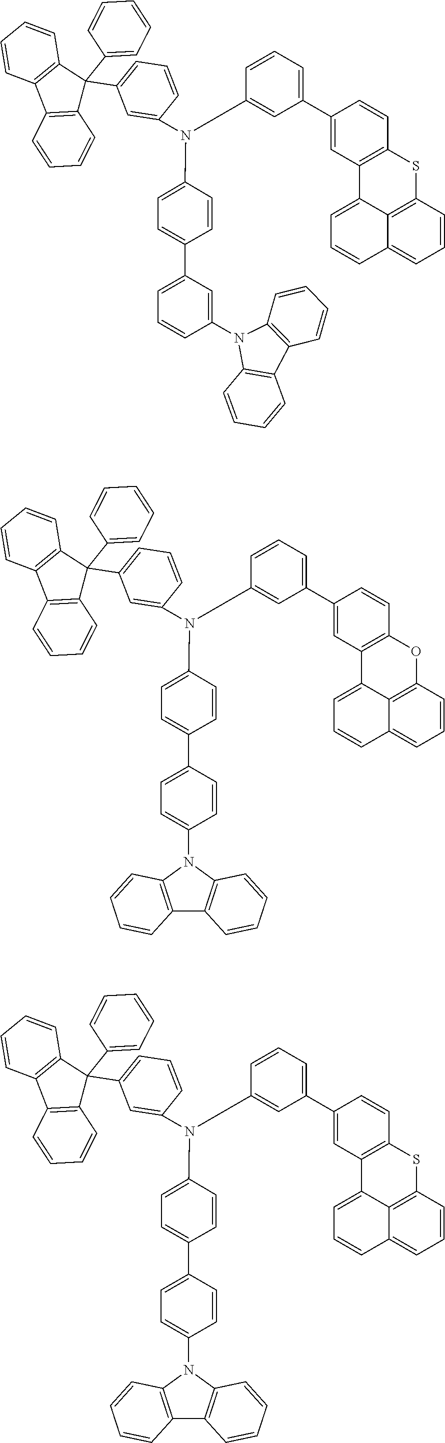 [SMILES: c1ccc(C2(c3cccc(N(c4ccc(-c5cccc(-n6c7ccccc7c7ccccc76)c5)cc4)c4cccc(-c5ccc6c(c5)-c5cccc7cccc(c57)S6)c4)c3)c3ccccc3-c3ccccc32)cc1 ; c1ccc(C2(c3cccc(N(c4ccc(-c5ccc(-n6c7ccccc7c7ccccc76)cc5)cc4)c4cccc(-c5ccc6c(c5)-c5cccc7cccc(c57)O6)c4)c3)c3ccccc3-c3ccccc32)cc1 ; c1ccc(C2(c3cccc(N(c4ccc(-c5ccc(-n6c7ccccc7c7ccccc76)cc5)cc4)c4cccc(-c5ccc6c(c5)-c5cccc7cccc(c57)S6)c4)c3)c3ccccc3-c3ccccc32)cc1]

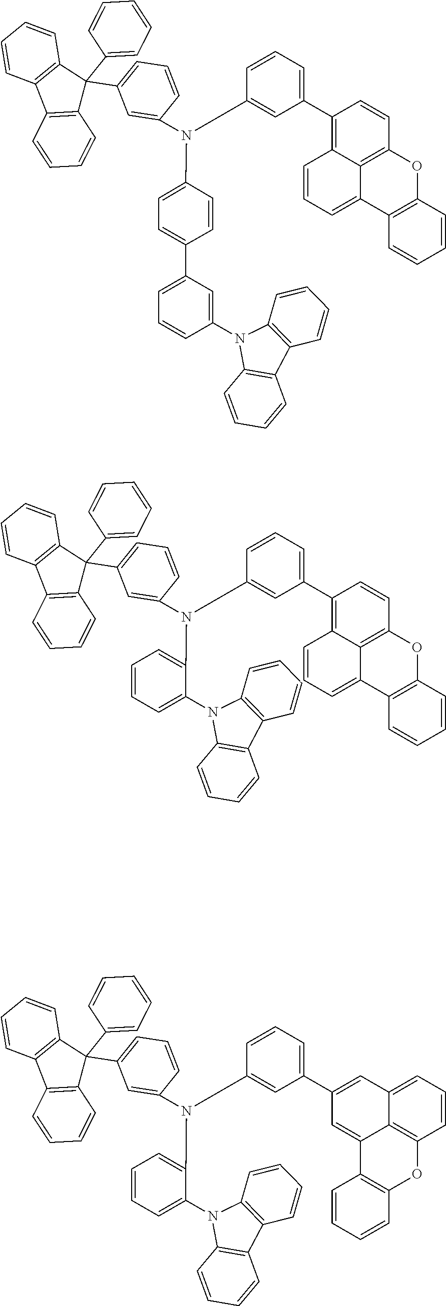 [SMILES: c1ccc(C2(c3cccc(N(c4ccc(-c5cccc(-n6c7ccccc7c7ccccc76)c5)cc4)c4cccc(-c5ccc6c7c(cccc57)-c5ccccc5O6)c4)c3)c3ccccc3-c3ccccc32)cc1 ; c1ccc(C2(c3cccc(N(c4cccc(-c5ccc6c7c(cccc57)-c5ccccc5O6)c4)c4ccccc4-n4c5ccccc5c5ccccc54)c3)c3ccccc3-c3ccccc32)cc1 ; c1ccc(C2(c3cccc(N(c4cccc(-c5cc6c7c(cccc7c5)Oc5ccccc5-6)c4)c4ccccc4-n4c5ccccc5c5ccccc54)c3)c3ccccc3-c3ccccc32)cc1]

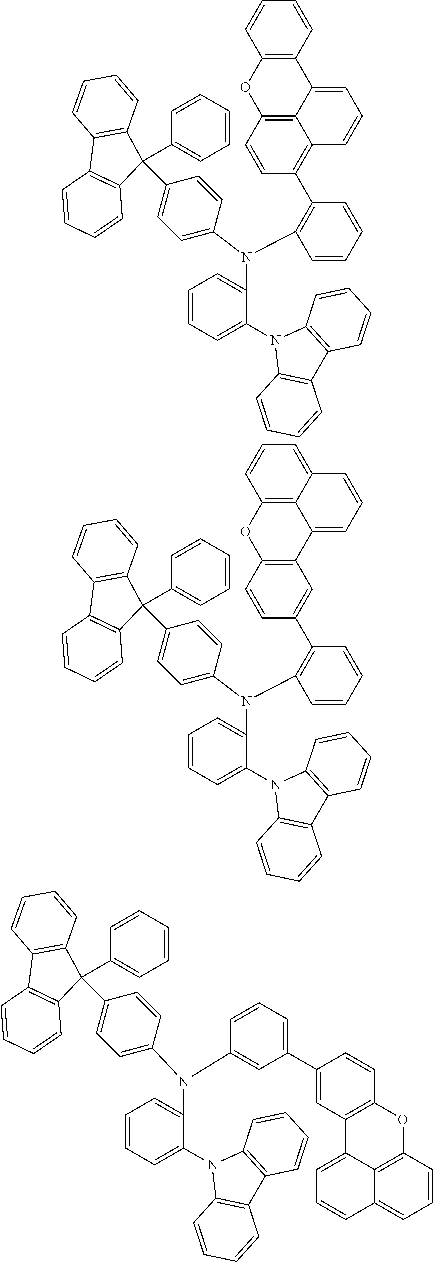

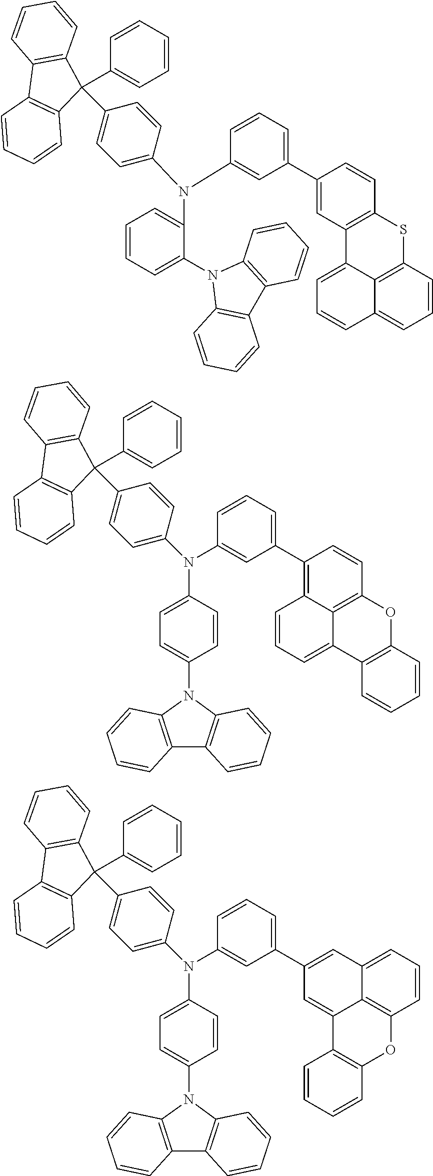

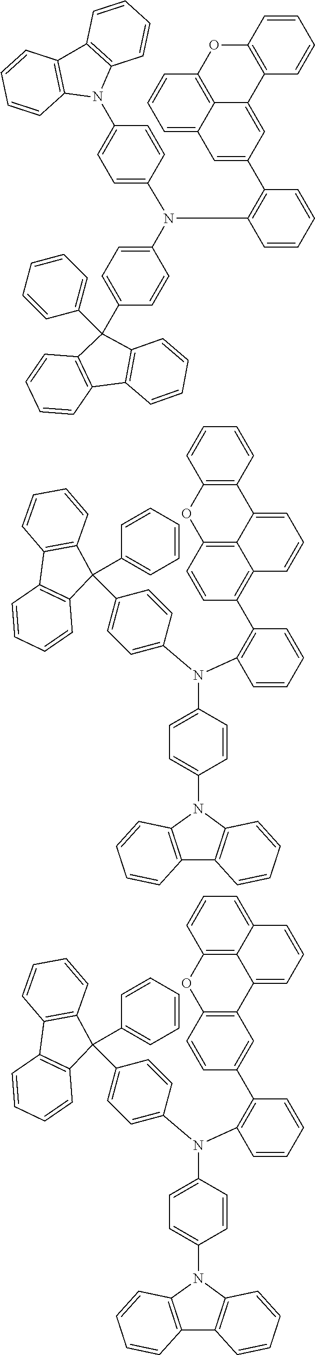

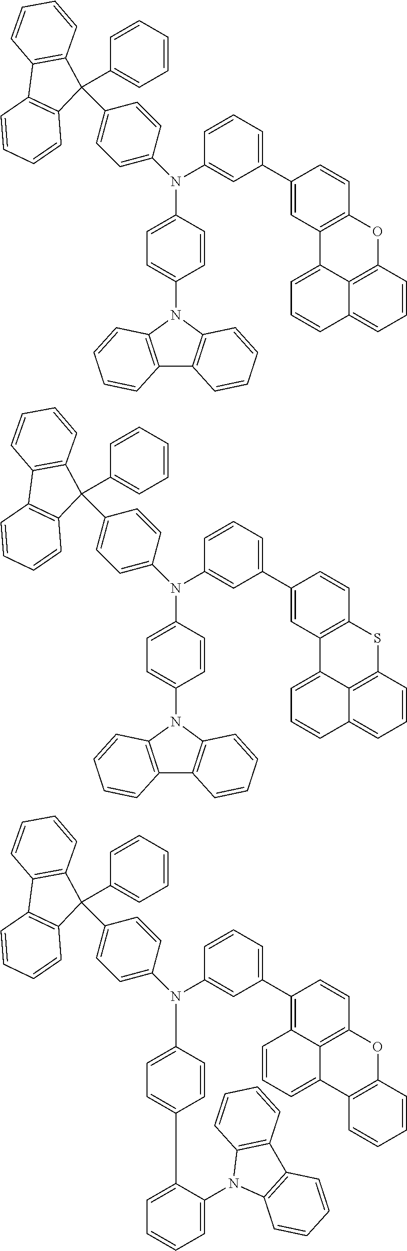

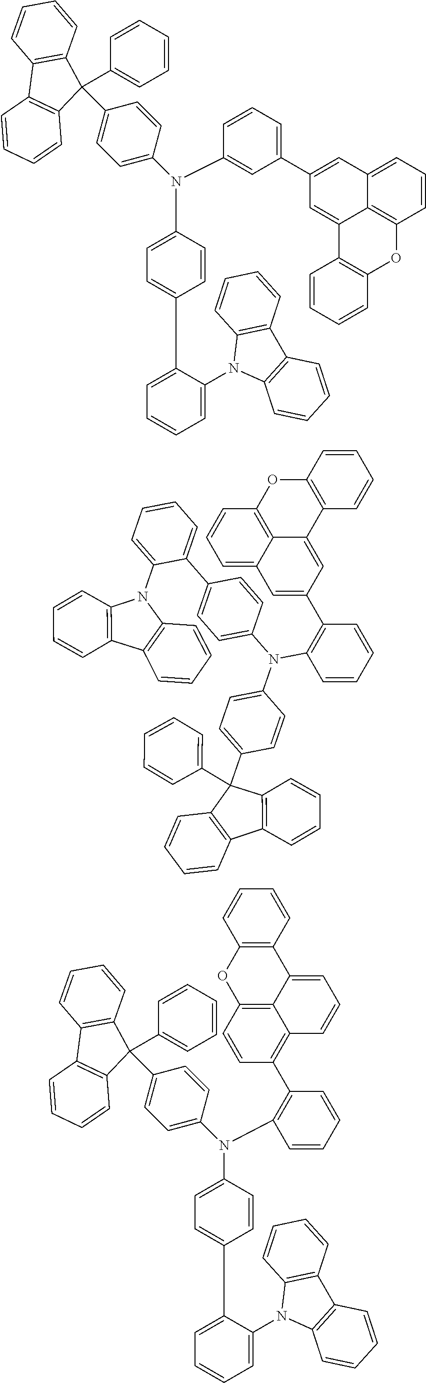

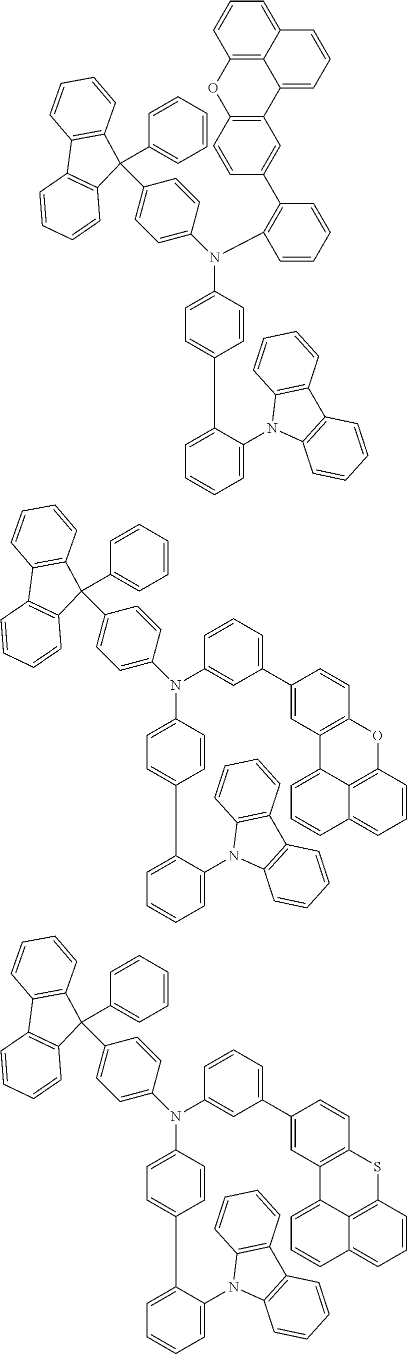 [SMILES: c1ccc(C2(c3ccc(N(c4ccc(-c5ccccc5-n5c6ccccc6c6ccccc65)cc4)c4ccccc4-c4ccc5c(c4)-c4cccc6cccc(c46)O5)cc3)c3ccccc3-c3ccccc32)cc1 ; c1ccc(C2(c3ccc(N(c4ccc(-c5ccccc5-n5c6ccccc6c6ccccc65)cc4)c4cccc(-c5ccc6c(c5)-c5cccc7cccc(c57)O6)c4)cc3)c3ccccc3-c3ccccc32)cc1 ; c1ccc(C2(c3ccc(N(c4ccc(-c5ccccc5-n5c6ccccc6c6ccccc65)cc4)c4cccc(-c5ccc6c(c5)-c5cccc7cccc(c57)S6)c4)cc3)c3ccccc3-c3ccccc32)cc1]

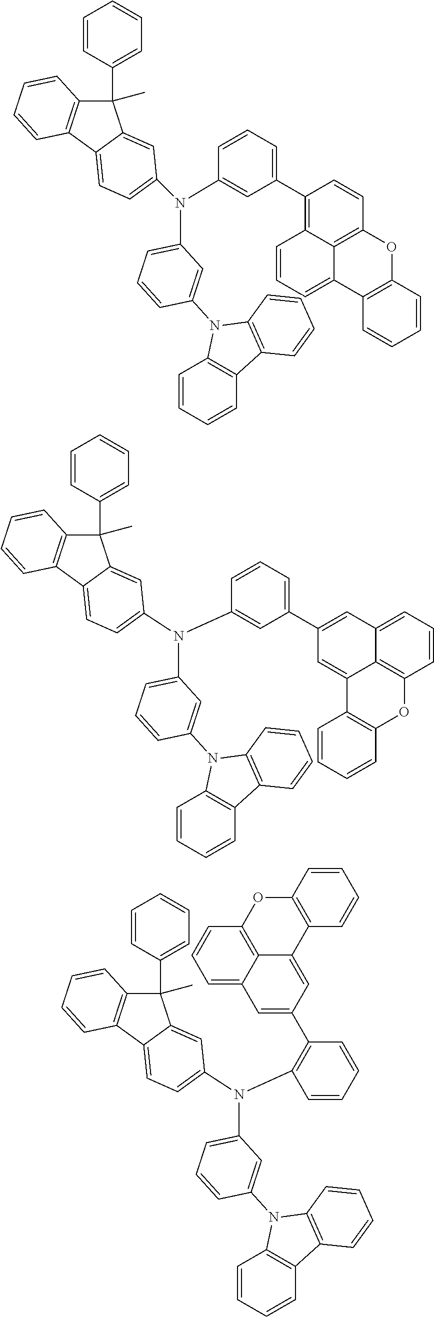 [SMILES: CC1(c2ccccc2)c2ccccc2-c2ccc(N(c3cccc(-c4ccc5c6c(cccc46)-c4ccccc4O5)c3)c3cccc(-n4c5ccccc5c5ccccc54)c3)cc21 ; CC1(c2ccccc2)c2ccccc2-c2ccc(N(c3cccc(-c4cc5c6c(cccc6c4)Oc4ccccc4-5)c3)c3cccc(-n4c5ccccc5c5ccccc54)c3)cc21 ; CC1(c2ccccc2)c2ccccc2-c2ccc(N(c3cccc(-n4c5ccccc5c5ccccc54)c3)c3ccccc3-c3cc4c5c(cccc5c3)Oc3ccccc3-4)cc21]

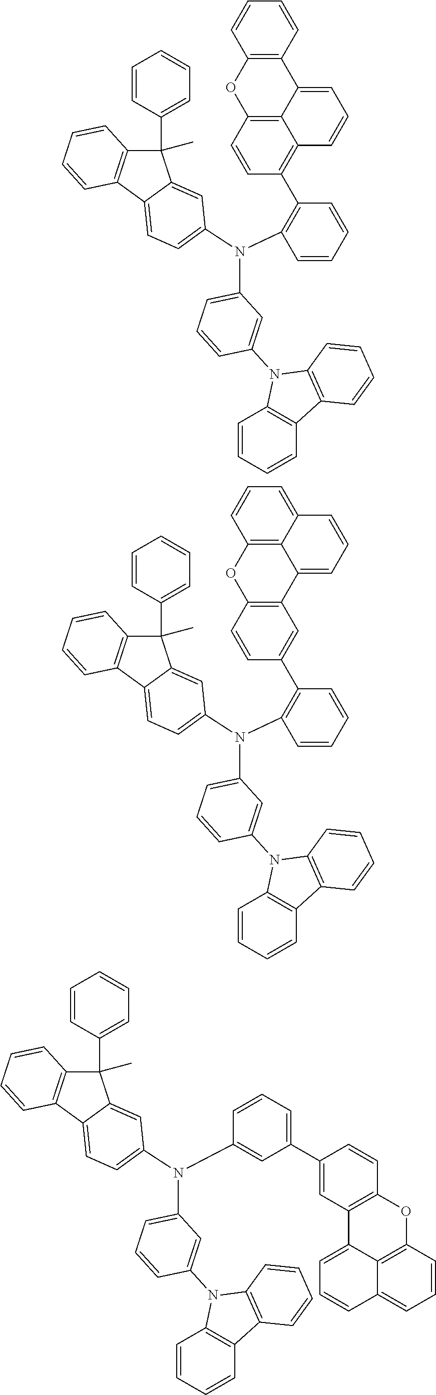

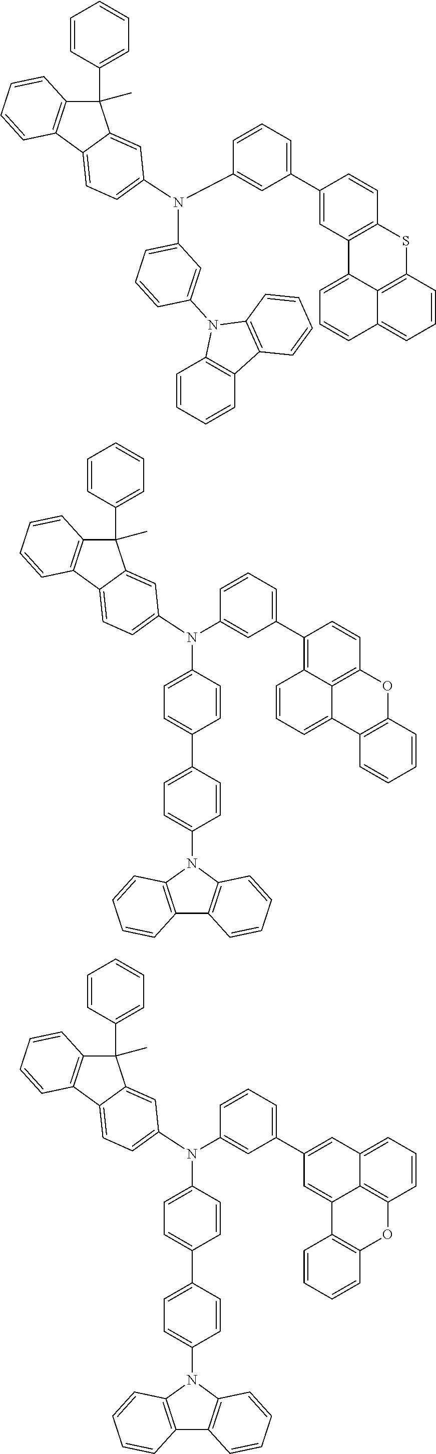

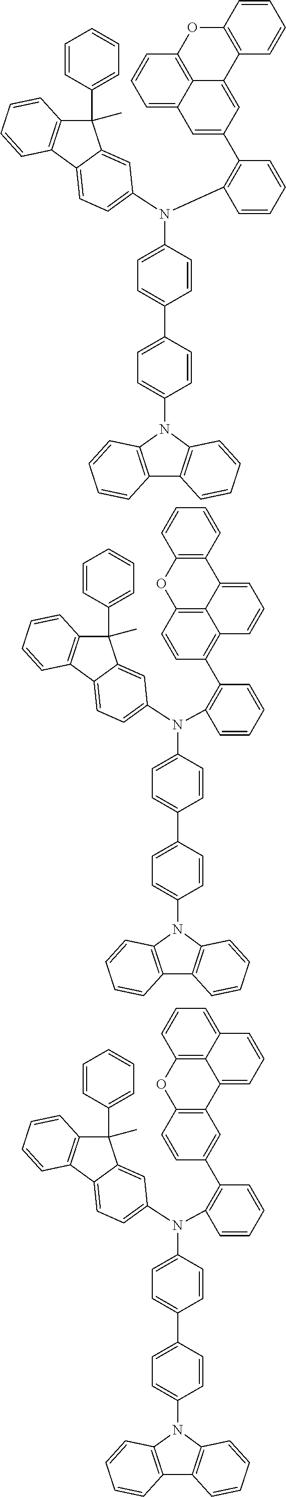

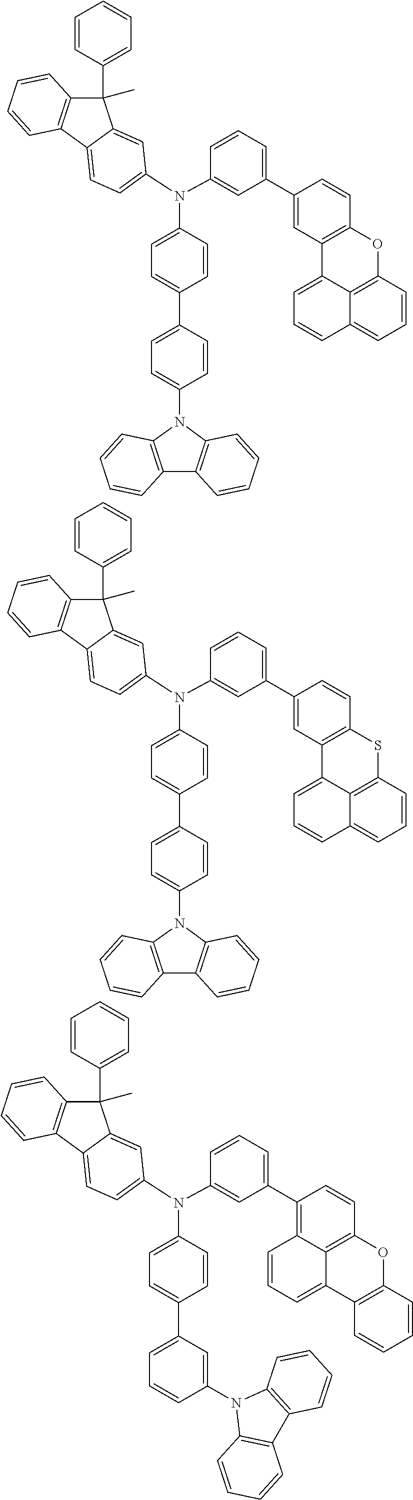

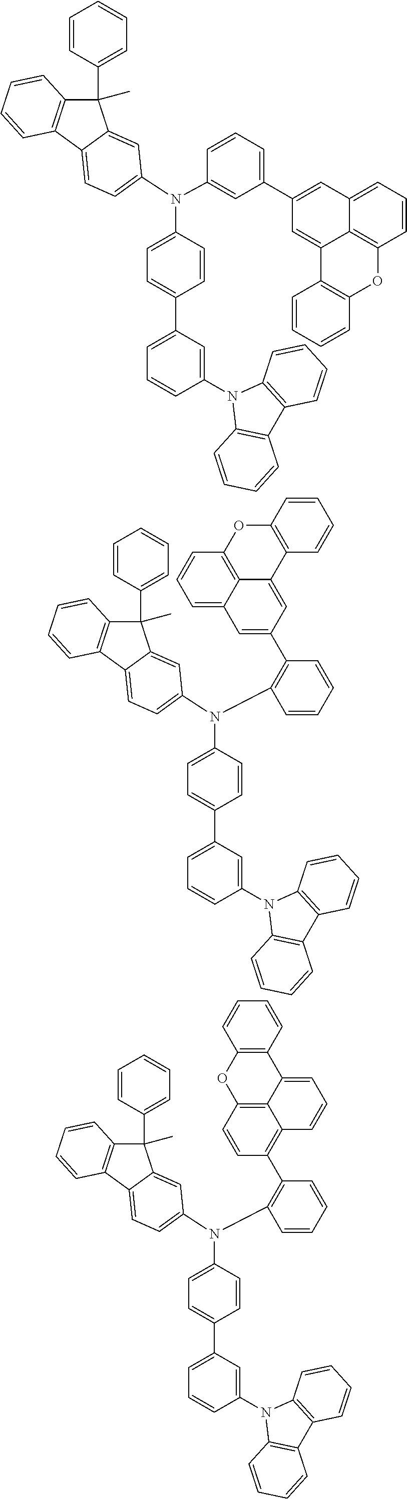

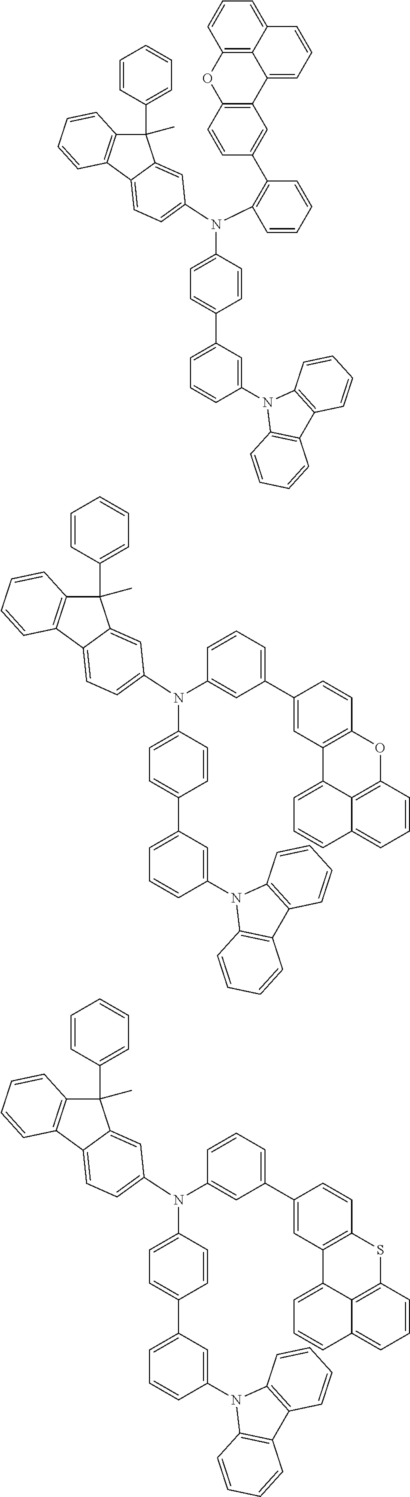 [SMILES: CC1(c2ccccc2)c2ccccc2-c2ccc(N(c3ccc(-c4cccc(-n5c6ccccc6c6ccccc65)c4)cc3)c3ccccc3-c3ccc4c(c3)-c3cccc5cccc(c35)O4)cc21 ; CC1(c2ccccc2)c2ccccc2-c2ccc(N(c3ccc(-c4cccc(-n5c6ccccc6c6ccccc65)c4)cc3)c3cccc(-c4ccc5c(c4)-c4cccc6cccc(c46)O5)c3)cc21 ; CC1(c2ccccc2)c2ccccc2-c2ccc(N(c3ccc(-c4cccc(-n5c6ccccc6c6ccccc65)c4)cc3)c3cccc(-c4ccc5c(c4)-c4cccc6cccc(c46)S5)c3)cc21]

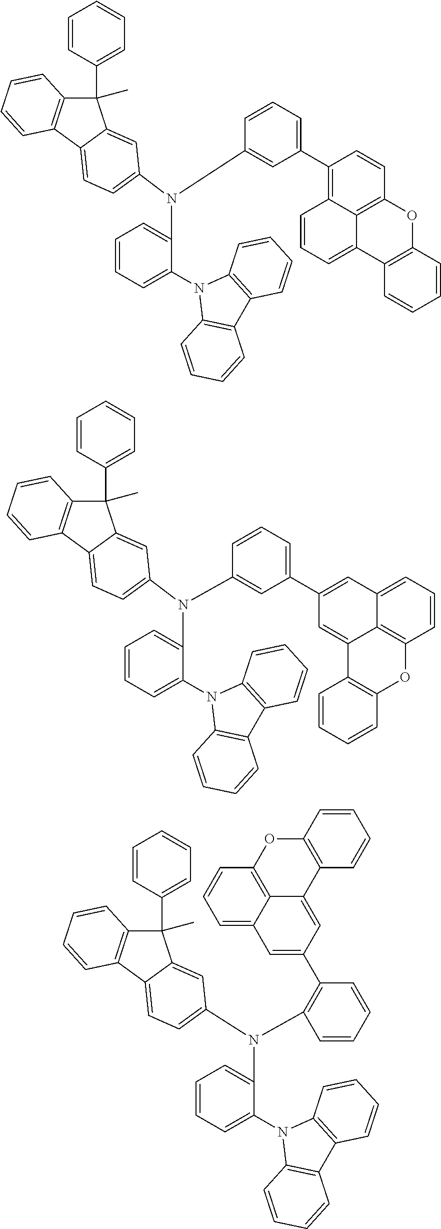 [SMILES: CC1(c2ccccc2)c2ccccc2-c2ccc(N(c3cccc(-c4ccc5c6c(cccc46)-c4ccccc4O5)c3)c3ccccc3-n3c4ccccc4c4ccccc43)cc21 ; CC1(c2ccccc2)c2ccccc2-c2ccc(N(c3cccc(-c4cc5c6c(cccc6c4)Oc4ccccc4-5)c3)c3ccccc3-n3c4ccccc4c4ccccc43)cc21 ; CC1(c2ccccc2)c2ccccc2-c2ccc(N(c3ccccc3-c3cc4c5c(cccc5c3)Oc3ccccc3-4)c3ccccc3-n3c4ccccc4c4ccccc43)cc21]

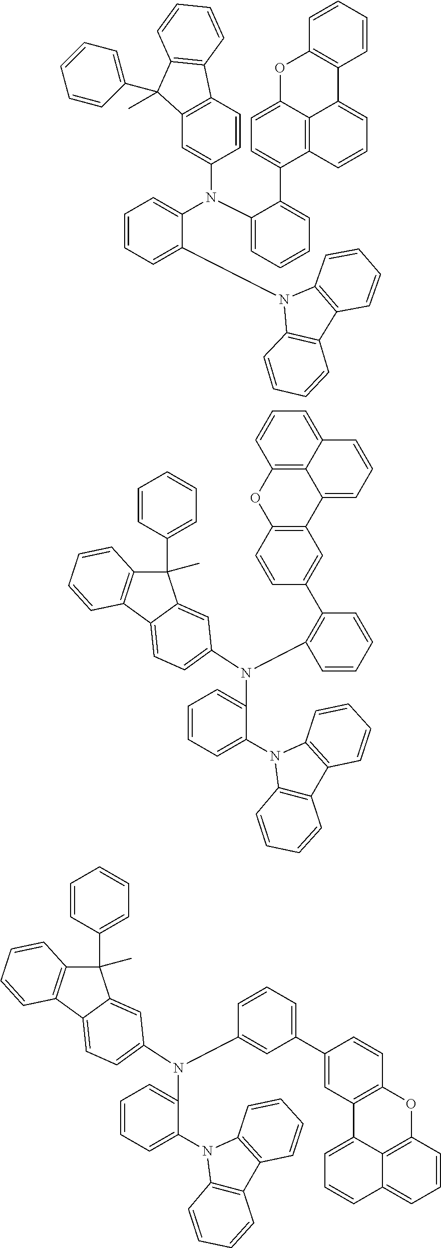

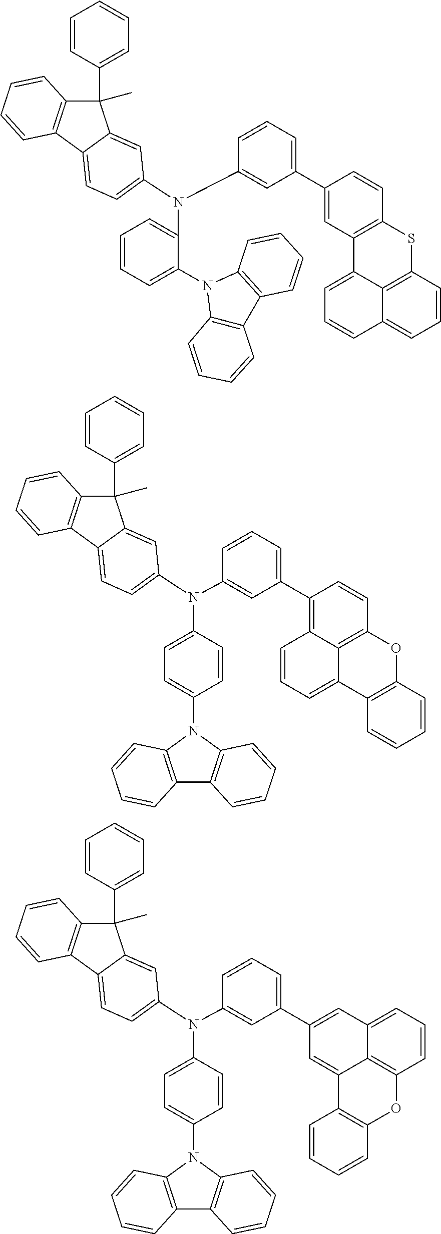

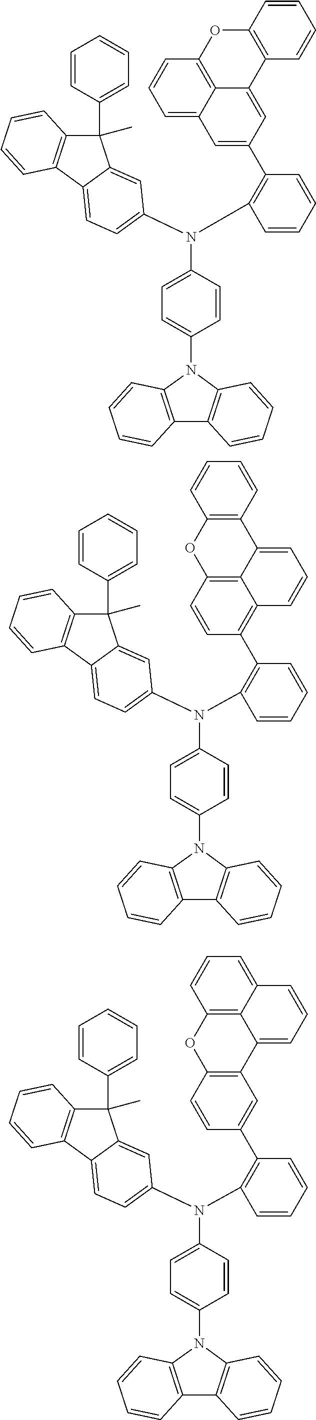

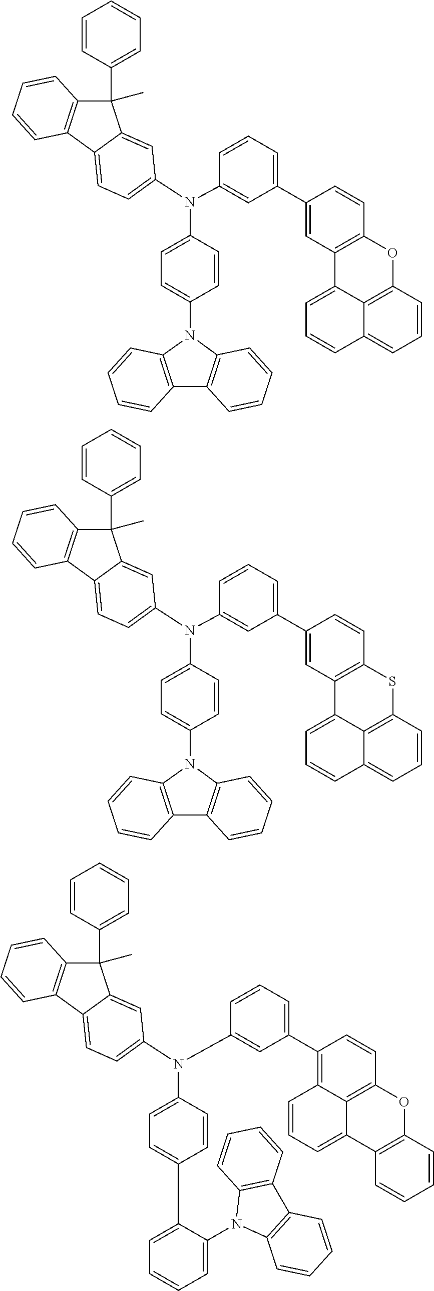

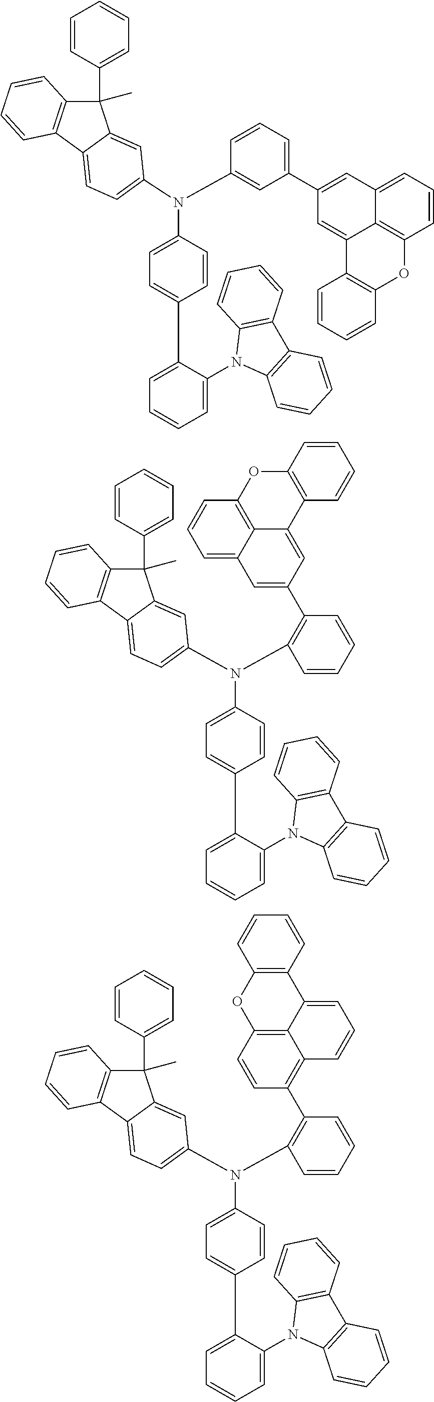

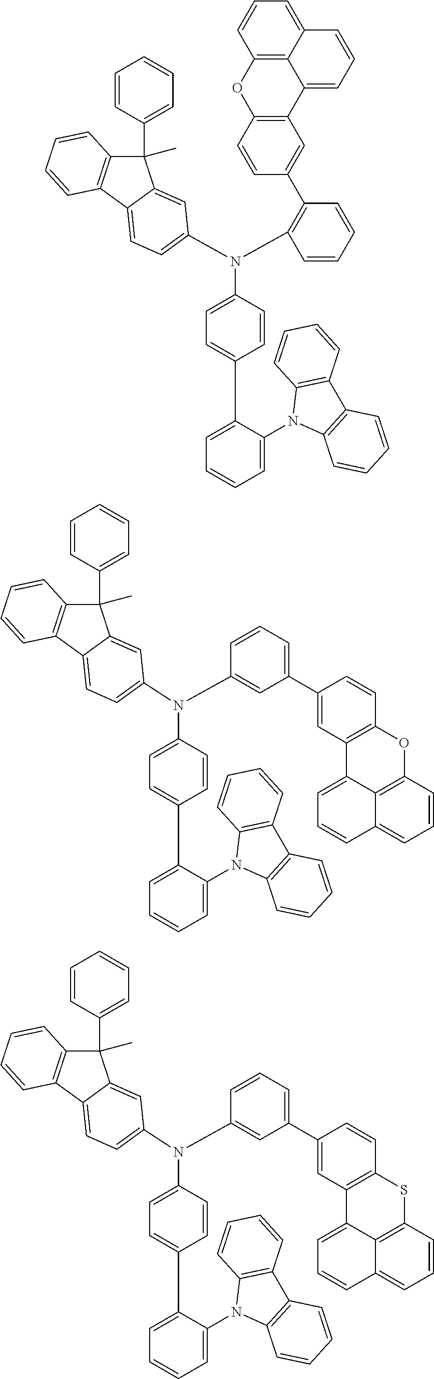 [SMILES: CC1(c2ccccc2)c2ccccc2-c2ccc(N(c3ccc(-c4ccccc4-n4c5ccccc5c5ccccc54)cc3)c3ccccc3-c3ccc4c(c3)-c3cccc5cccc(c35)O4)cc21 ; CC1(c2ccccc2)c2ccccc2-c2ccc(N(c3ccc(-c4ccccc4-n4c5ccccc5c5ccccc54)cc3)c3cccc(-c4ccc5c(c4)-c4cccc6cccc(c46)O5)c3)cc21 ; CC1(c2ccccc2)c2ccccc2-c2ccc(N(c3ccc(-c4ccccc4-n4c5ccccc5c5ccccc54)cc3)c3cccc(-c4ccc5c(c4)-c4cccc6cccc(c46)S5)c3)cc21]

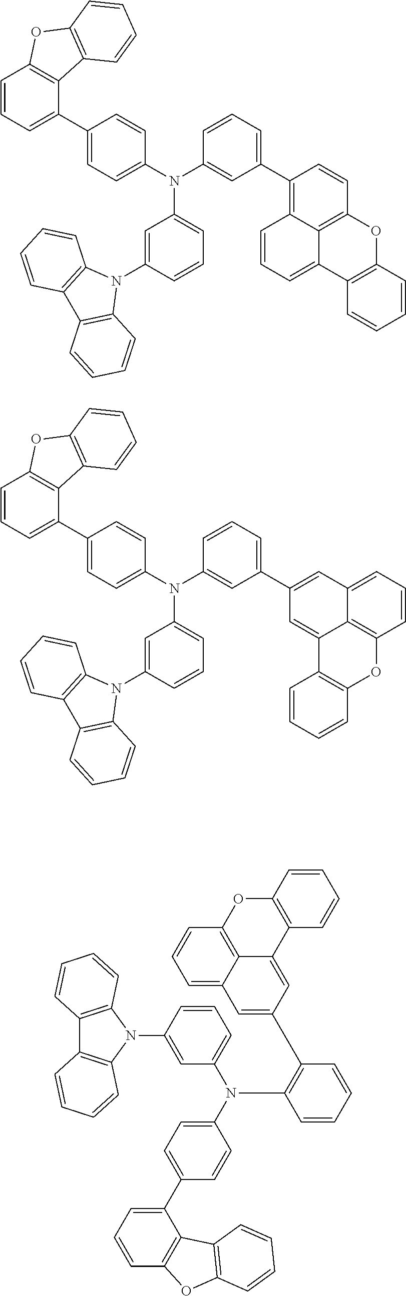

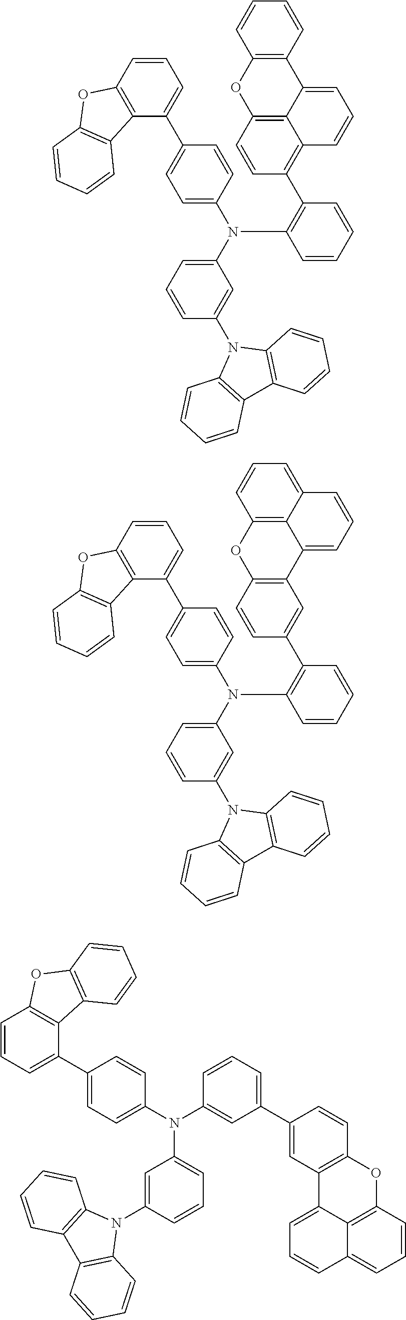

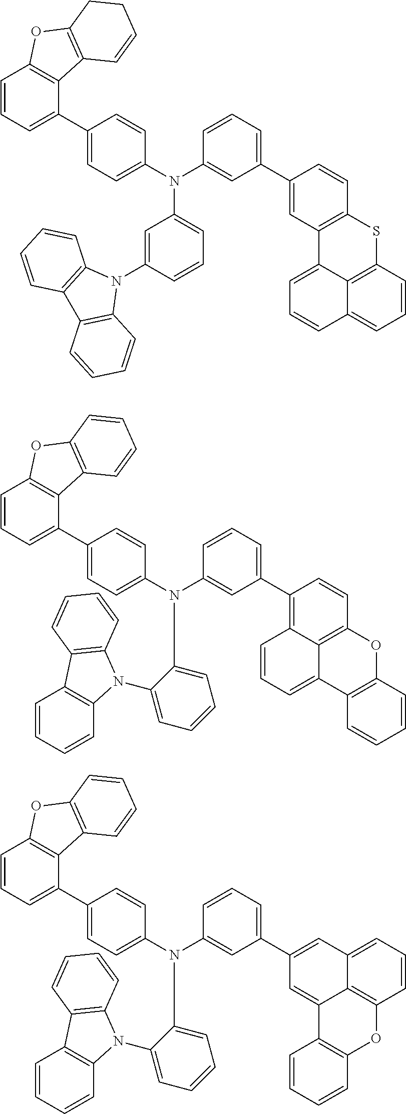

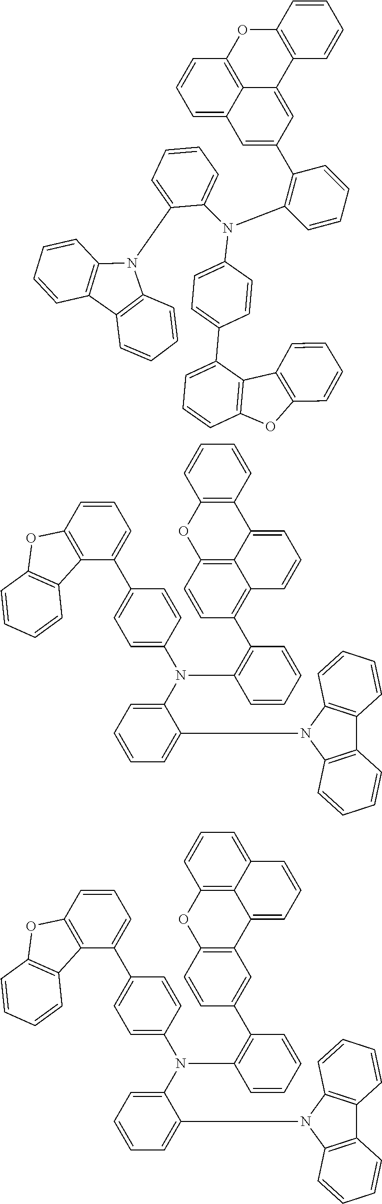

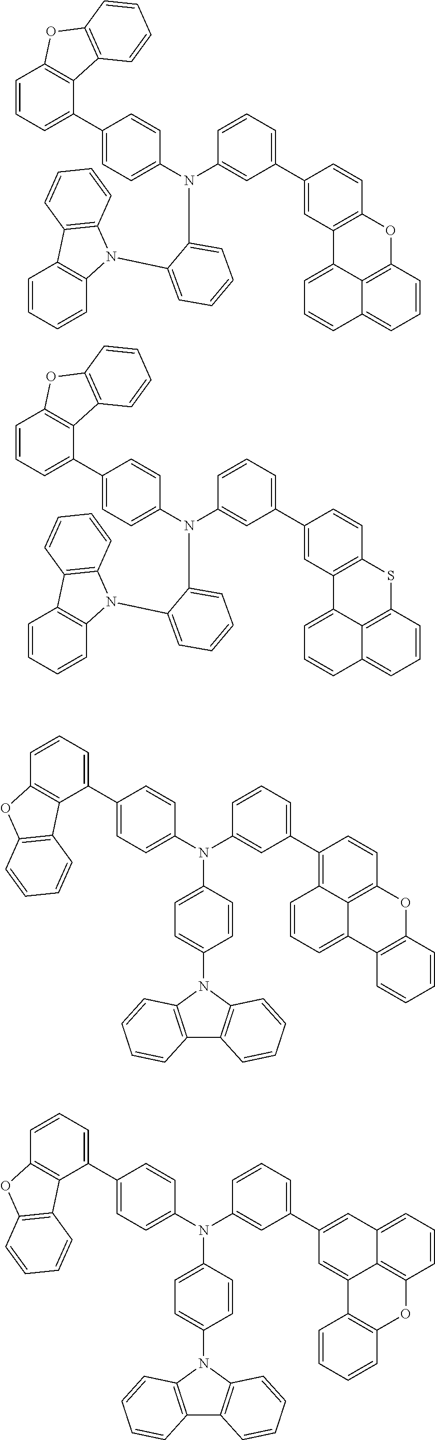

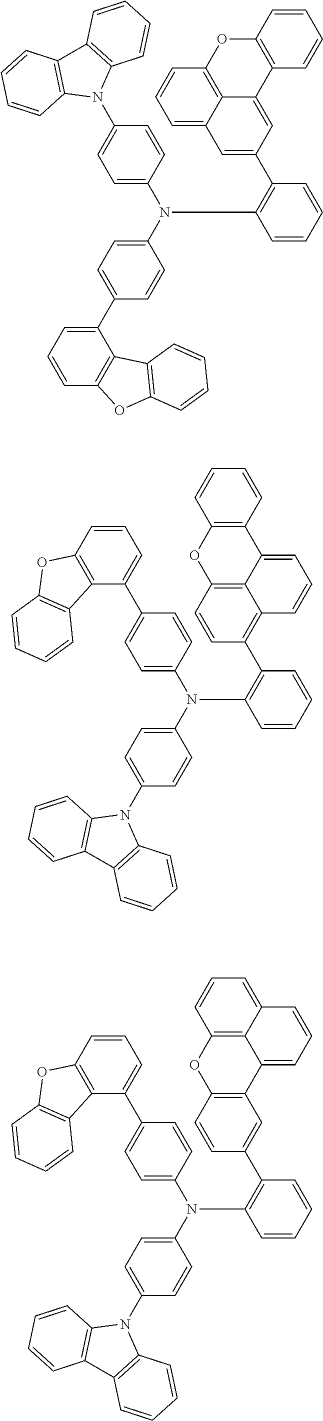 [SMILES: c1ccc2c(c1)Oc1cccc3cc(-c4ccccc4N(c4ccc(-c5cccc6oc7ccccc7c56)cc4)c4ccc(-n5c6ccccc6c6ccccc65)cc4)cc-2c13 ; c1ccc2c(c1)Oc1ccc(-c3ccccc3N(c3ccc(-c4cccc5oc6ccccc6c45)cc3)c3ccc(-n4c5ccccc5c5ccccc54)cc3)c3cccc-2c13 ; c1ccc(N(c2ccc(-c3cccc4oc5ccccc5c34)cc2)c2ccc(-n3c4ccccc4c4ccccc43)cc2)c(-c2ccc3c(c2)-c2cccc4cccc(c24)O3)c1]

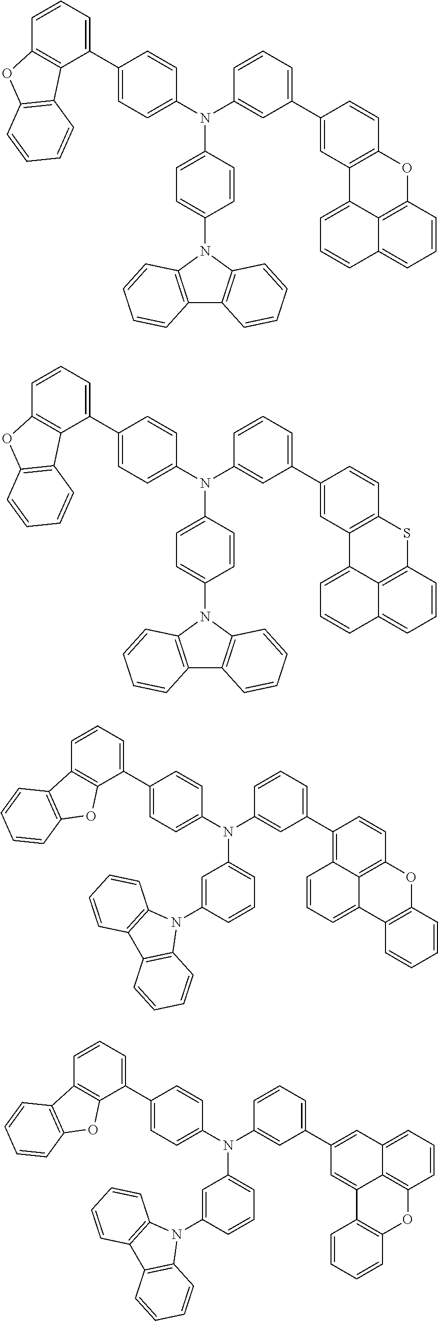 [SMILES: c1cc(-c2ccc3c(c2)-c2cccc4cccc(c24)O3)cc(N(c2ccc(-c3cccc4oc5ccccc5c34)cc2)c2ccc(-n3c4ccccc4c4ccccc43)cc2)c1 ; c1cc(-c2ccc3c(c2)-c2cccc4cccc(c24)S3)cc(N(c2ccc(-c3cccc4oc5ccccc5c34)cc2)c2ccc(-n3c4ccccc4c4ccccc43)cc2)c1 ; c1cc(-c2ccc3c4c(cccc24)-c2ccccc2O3)cc(N(c2ccc(-c3cccc4c3oc3ccccc34)cc2)c2cccc(-n3c4ccccc4c4ccccc43)c2)c1 ; c1cc(-c2cc3c4c(cccc4c2)Oc2ccccc2-3)cc(N(c2ccc(-c3cccc4c3oc3ccccc34)cc2)c2cccc(-n3c4ccccc4c4ccccc43)c2)c1]

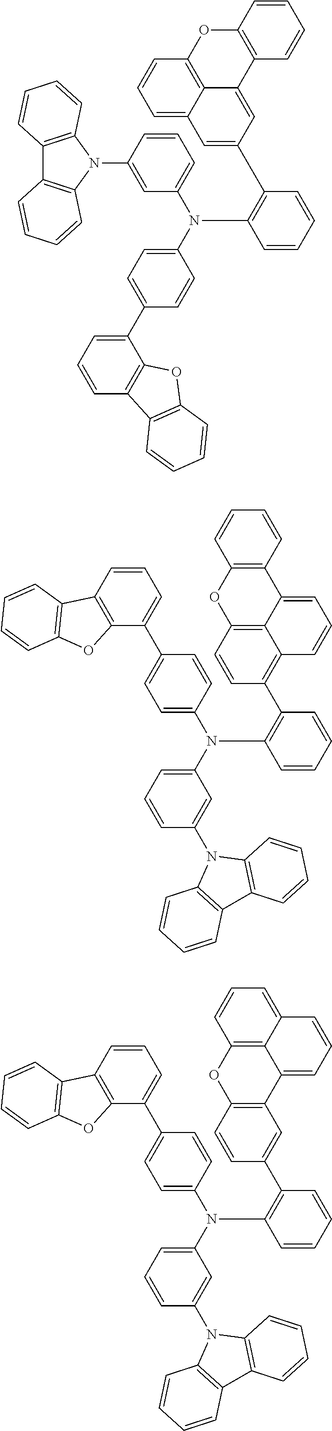 [SMILES: c1cc(N(c2ccc(-c3cccc4c3oc3ccccc34)cc2)c2ccccc2-c2cc3c4c(cccc4c2)Oc2ccccc2-3)cc(-n2c3ccccc3c3ccccc32)c1 ; c1cc(N(c2ccc(-c3cccc4c3oc3ccccc34)cc2)c2ccccc2-c2ccc3c4c(cccc24)-c2ccccc2O3)cc(-n2c3ccccc3c3ccccc32)c1 ; c1cc(N(c2ccc(-c3cccc4c3oc3ccccc34)cc2)c2ccccc2-c2ccc3c(c2)-c2cccc4cccc(c24)O3)cc(-n2c3ccccc3c3ccccc32)c1]

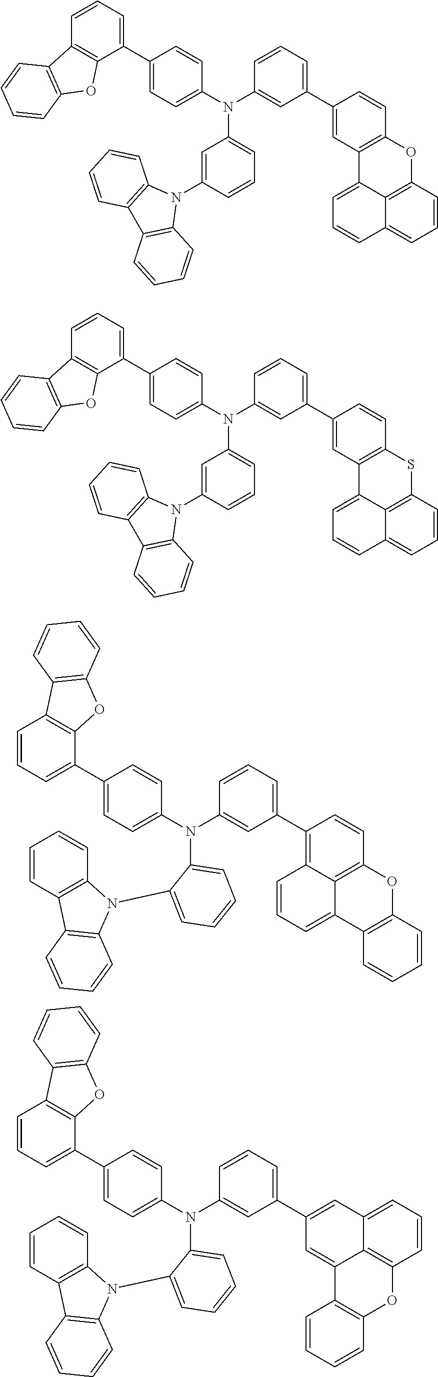

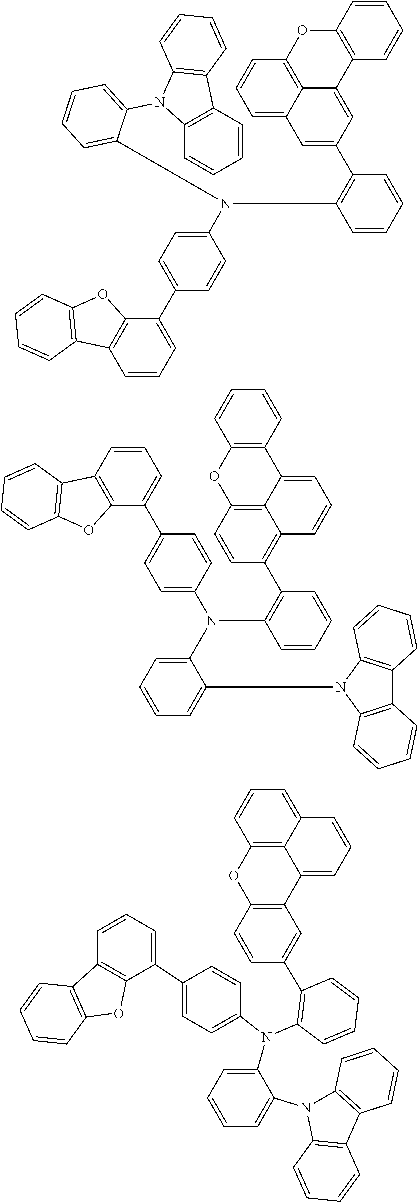 [SMILES: c1ccc2c(c1)Oc1cccc3cc(-c4ccccc4N(c4ccc(-c5cccc6c5oc5ccccc56)cc4)c4ccccc4-n4c5ccccc5c5ccccc54)cc-2c13 ; c1ccc2c(c1)Oc1ccc(-c3ccccc3N(c3ccc(-c4cccc5c4oc4ccccc45)cc3)c3ccccc3-n3c4ccccc4c4ccccc43)c3cccc-2c13 ; c1ccc(N(c2ccc(-c3cccc4c3oc3ccccc34)cc2)c2ccccc2-n2c3ccccc3c3ccccc32)c(-c2ccc3c(c2)-c2cccc4cccc(c24)O3)c1]

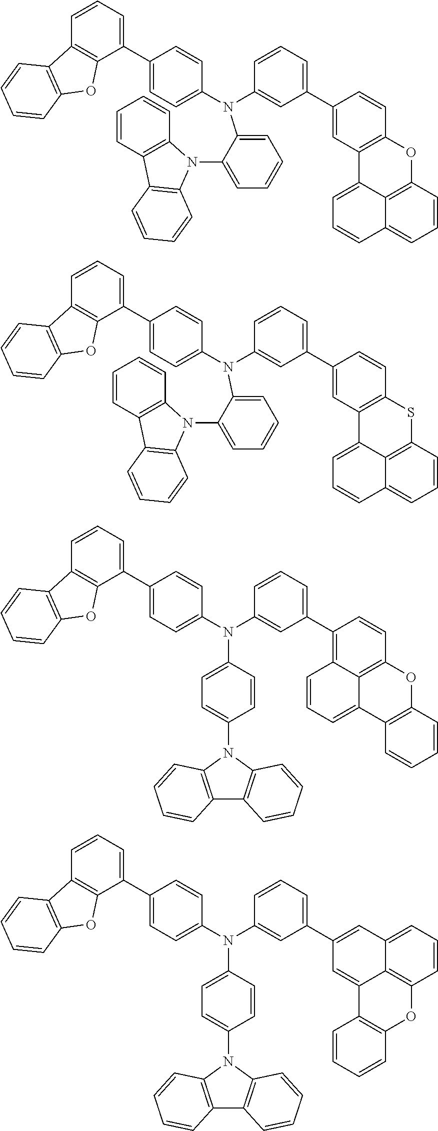 [SMILES: c1cc(-c2ccc3c(c2)-c2cccc4cccc(c24)O3)cc(N(c2ccc(-c3cccc4c3oc3ccccc34)cc2)c2ccccc2-n2c3ccccc3c3ccccc32)c1 ; c1cc(-c2ccc3c(c2)-c2cccc4cccc(c24)S3)cc(N(c2ccc(-c3cccc4c3oc3ccccc34)cc2)c2ccccc2-n2c3ccccc3c3ccccc32)c1 ; c1cc(-c2ccc3c4c(cccc24)-c2ccccc2O3)cc(N(c2ccc(-c3cccc4c3oc3ccccc34)cc2)c2ccc(-n3c4ccccc4c4ccccc43)cc2)c1 ; c1cc(-c2cc3c4c(cccc4c2)Oc2ccccc2-3)cc(N(c2ccc(-c3cccc4c3oc3ccccc34)cc2)c2ccc(-n3c4ccccc4c4ccccc43)cc2)c1]

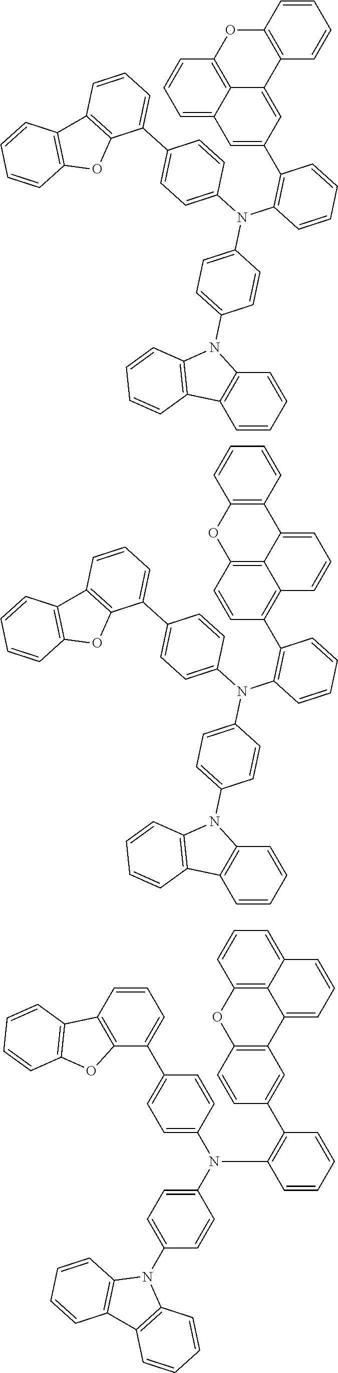

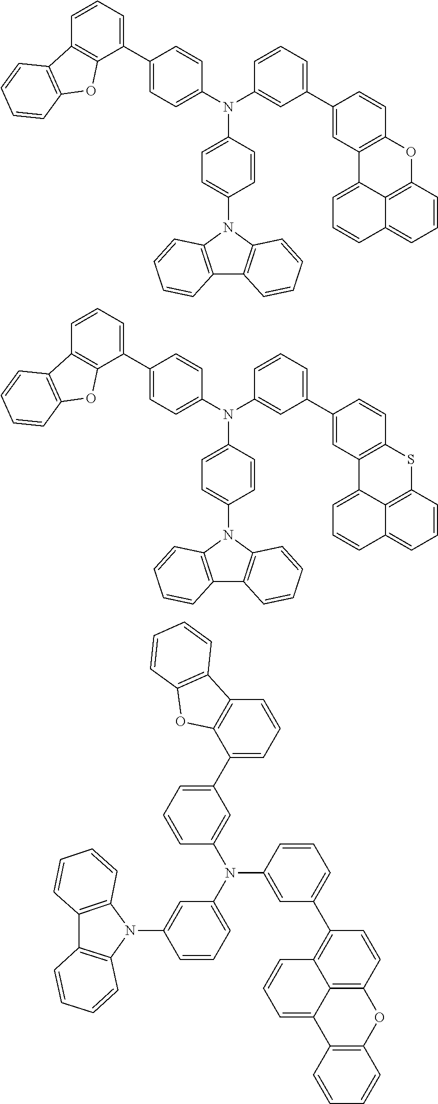

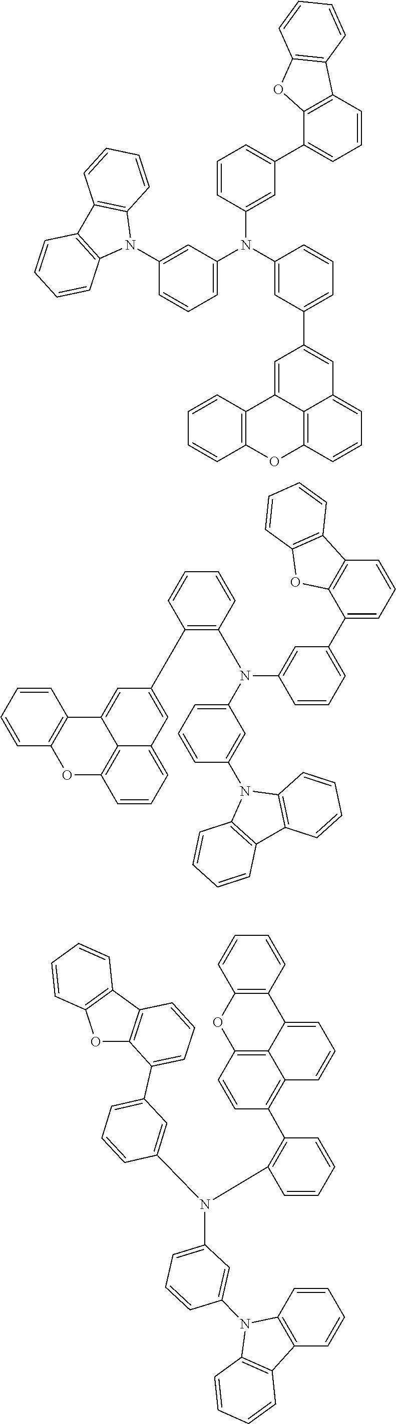

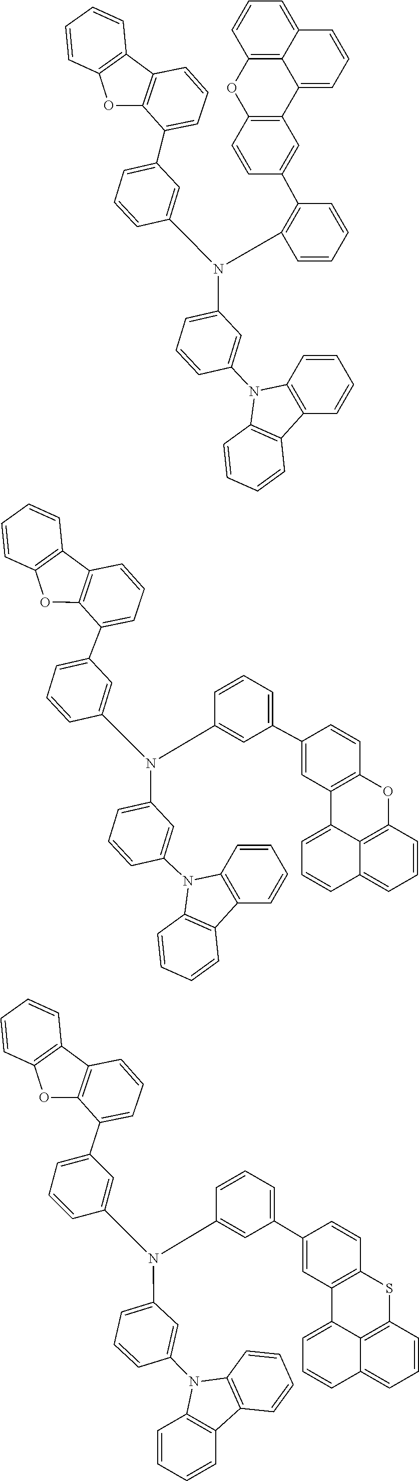 [SMILES: c1cc(-c2cccc3c2oc2ccccc23)cc(N(c2cccc(-n3c4ccccc4c4ccccc43)c2)c2ccccc2-c2ccc3c(c2)-c2cccc4cccc(c24)O3)c1 ; c1cc(-c2ccc3c(c2)-c2cccc4cccc(c24)O3)cc(N(c2cccc(-c3cccc4c3oc3ccccc34)c2)c2cccc(-n3c4ccccc4c4ccccc43)c2)c1 ; c1cc(-c2ccc3c(c2)-c2cccc4cccc(c24)S3)cc(N(c2cccc(-c3cccc4c3oc3ccccc34)c2)c2cccc(-n3c4ccccc4c4ccccc43)c2)c1]

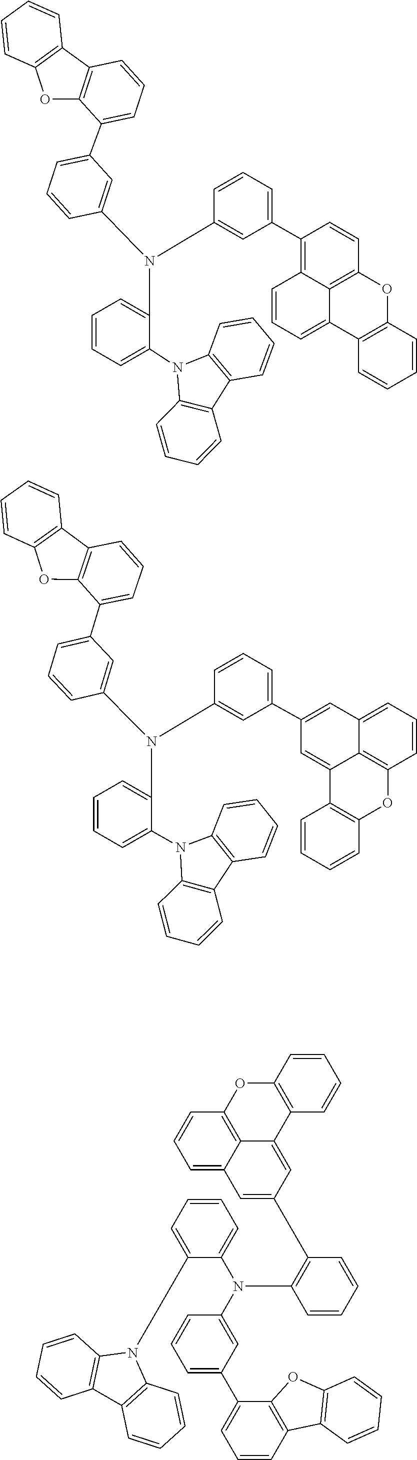 [SMILES: c1cc(-c2ccc3c4c(cccc24)-c2ccccc2O3)cc(N(c2cccc(-c3cccc4c3oc3ccccc34)c2)c2ccccc2-n2c3ccccc3c3ccccc32)c1 ; c1cc(-c2cc3c4c(cccc4c2)Oc2ccccc2-3)cc(N(c2cccc(-c3cccc4c3oc3ccccc34)c2)c2ccccc2-n2c3ccccc3c3ccccc32)c1 ; c1cc(-c2cccc3c2oc2ccccc23)cc(N(c2ccccc2-c2cc3c4c(cccc4c2)Oc2ccccc2-3)c2ccccc2-n2c3ccccc3c3ccccc32)c1]

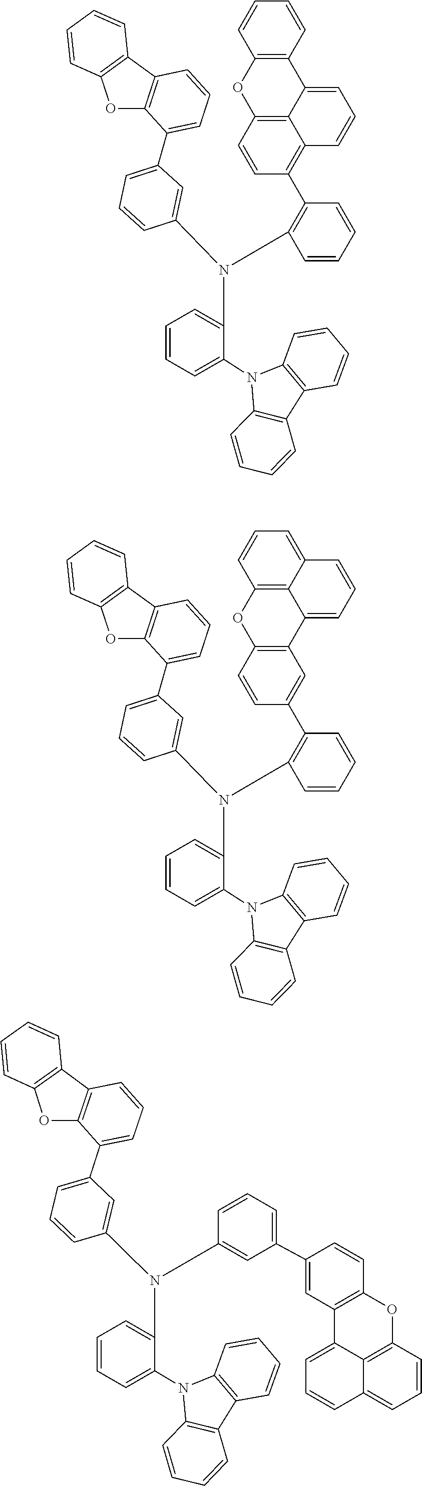 [SMILES: c1cc(-c2cccc3c2oc2ccccc23)cc(N(c2ccccc2-c2ccc3c4c(cccc24)-c2ccccc2O3)c2ccccc2-n2c3ccccc3c3ccccc32)c1 ; c1cc(-c2cccc3c2oc2ccccc23)cc(N(c2ccccc2-c2ccc3c(c2)-c2cccc4cccc(c24)O3)c2ccccc2-n2c3ccccc3c3ccccc32)c1 ; c1cc(-c2ccc3c(c2)-c2cccc4cccc(c24)O3)cc(N(c2cccc(-c3cccc4c3oc3ccccc34)c2)c2ccccc2-n2c3ccccc3c3ccccc32)c1]

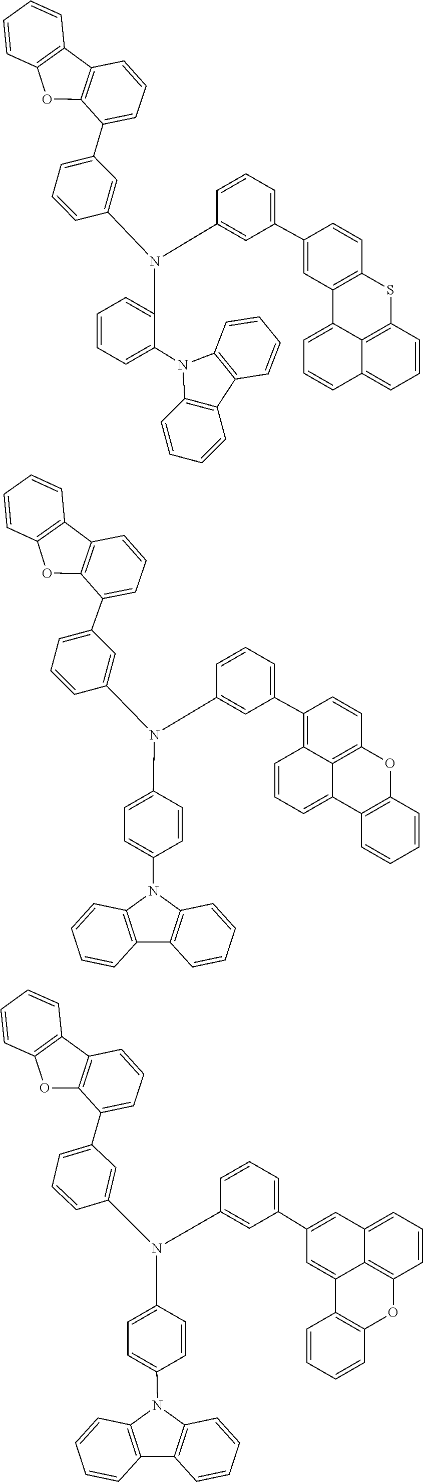 [SMILES: c1cc(-c2ccc3c(c2)-c2cccc4cccc(c24)S3)cc(N(c2cccc(-c3cccc4c3oc3ccccc34)c2)c2ccccc2-n2c3ccccc3c3ccccc32)c1 ; c1cc(-c2ccc3c4c(cccc24)-c2ccccc2O3)cc(N(c2ccc(-n3c4ccccc4c4ccccc43)cc2)c2cccc(-c3cccc4c3oc3ccccc34)c2)c1 ; c1cc(-c2cc3c4c(cccc4c2)Oc2ccccc2-3)cc(N(c2ccc(-n3c4ccccc4c4ccccc43)cc2)c2cccc(-c3cccc4c3oc3ccccc34)c2)c1]

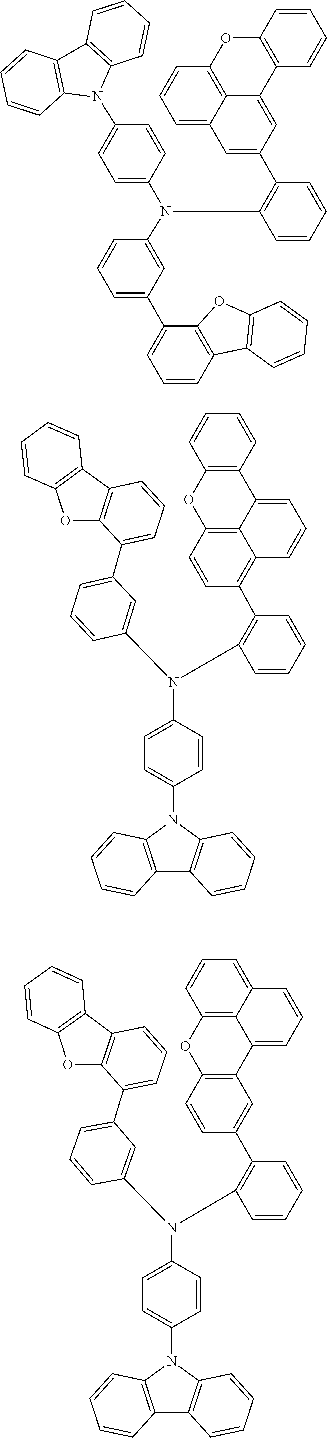

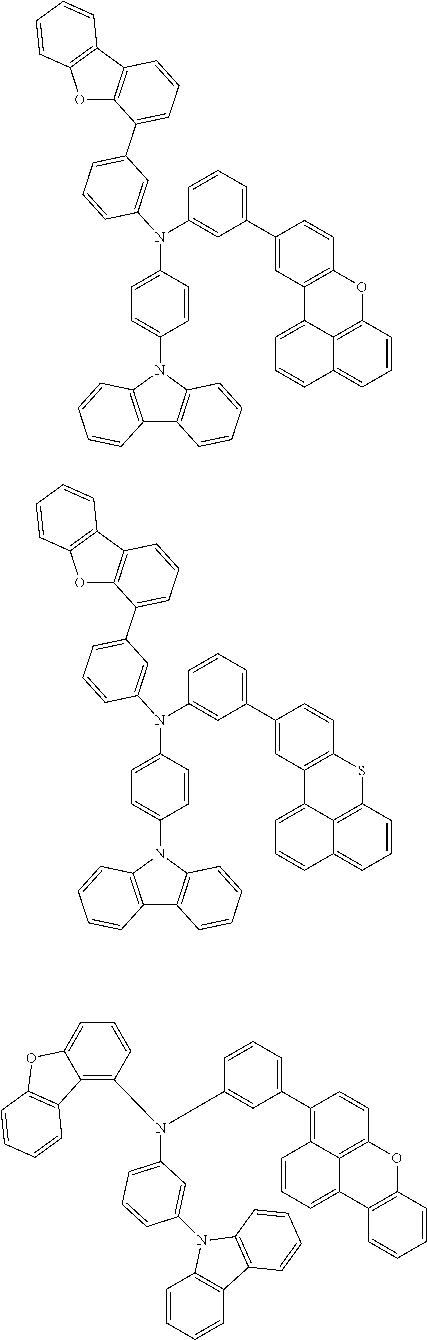 [SMILES: c1cc(-c2ccc3c(c2)-c2cccc4cccc(c24)O3)cc(N(c2ccc(-n3c4ccccc4c4ccccc43)cc2)c2cccc(-c3cccc4c3oc3ccccc34)c2)c1 ; c1cc(-c2ccc3c(c2)-c2cccc4cccc(c24)S3)cc(N(c2ccc(-n3c4ccccc4c4ccccc43)cc2)c2cccc(-c3cccc4c3oc3ccccc34)c2)c1 ; c1cc(-c2ccc3c4c(cccc24)-c2ccccc2O3)cc(N(c2cccc(-n3c4ccccc4c4ccccc43)c2)c2cccc3oc4ccccc4c23)c1]

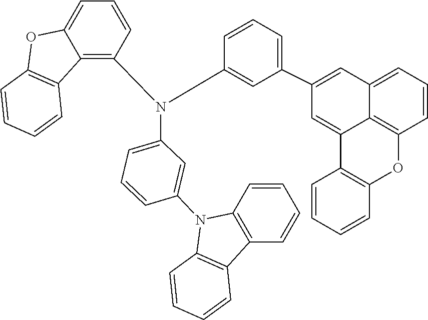 [SMILES: c1cc(-c2cc3c4c(cccc4c2)Oc2ccccc2-3)cc(N(c2cccc(-n3c4ccccc4c4ccccc43)c2)c2cccc3oc4ccccc4c23)c1]

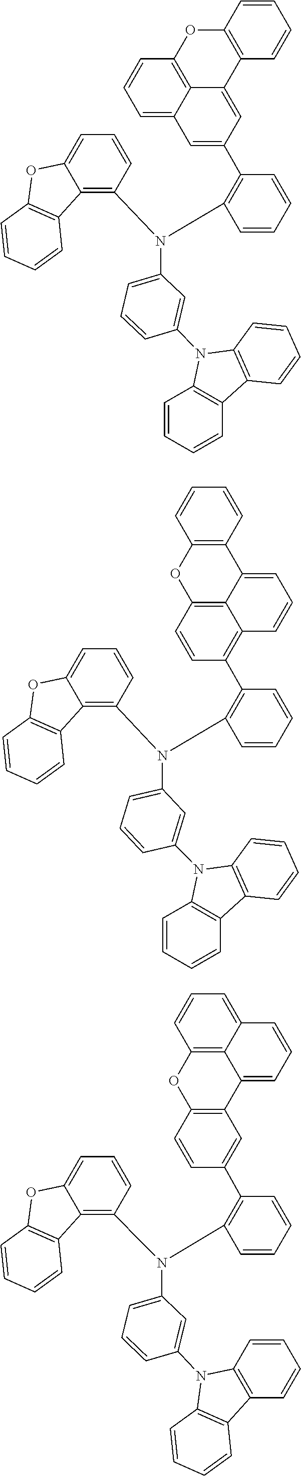

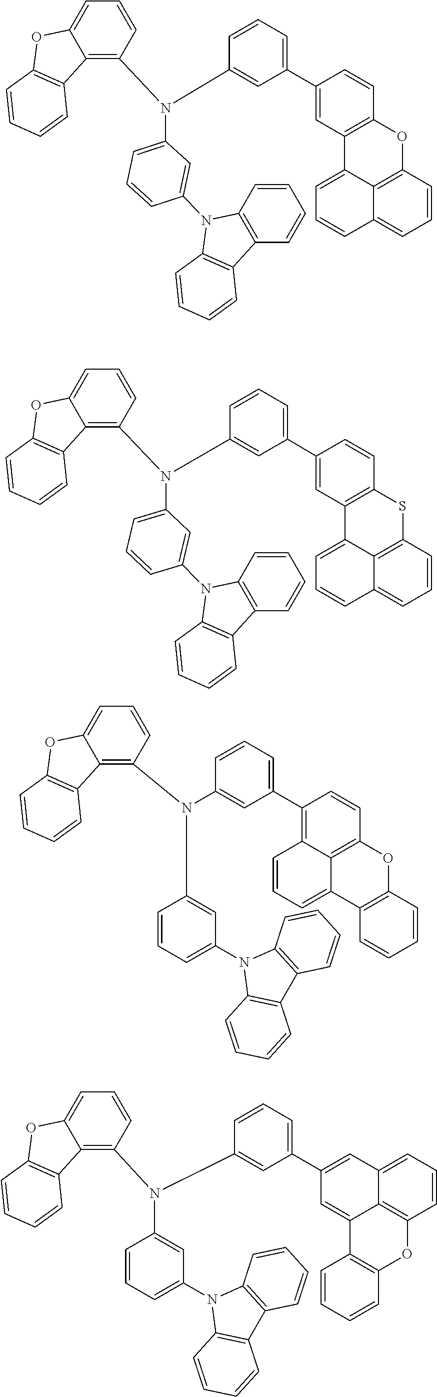

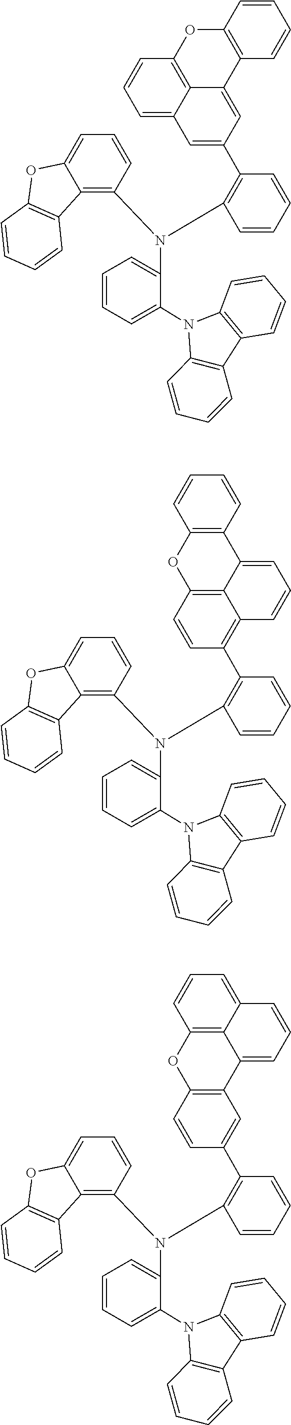 [SMILES: c1ccc2c(c1)Oc1cccc3cc(-c4ccccc4N(c4ccccc4-n4c5ccccc5c5ccccc54)c4cccc5oc6ccccc6c45)cc-2c13 ; c1ccc2c(c1)Oc1ccc(-c3ccccc3N(c3ccccc3-n3c4ccccc4c4ccccc43)c3cccc4oc5ccccc5c34)c3cccc-2c13 ; c1ccc(N(c2ccccc2-n2c3ccccc3c3ccccc32)c2cccc3oc4ccccc4c23)c(-c2ccc3c(c2)-c2cccc4cccc(c24)O3)c1]

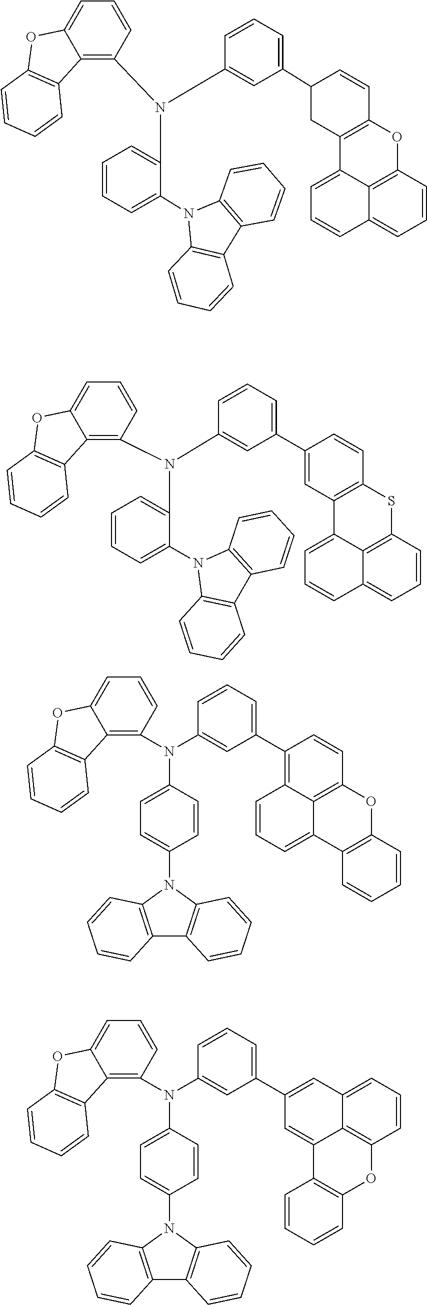

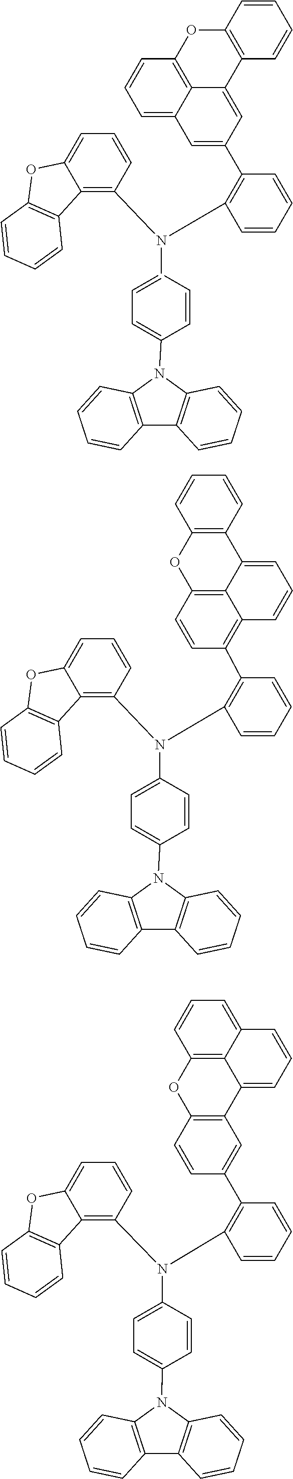

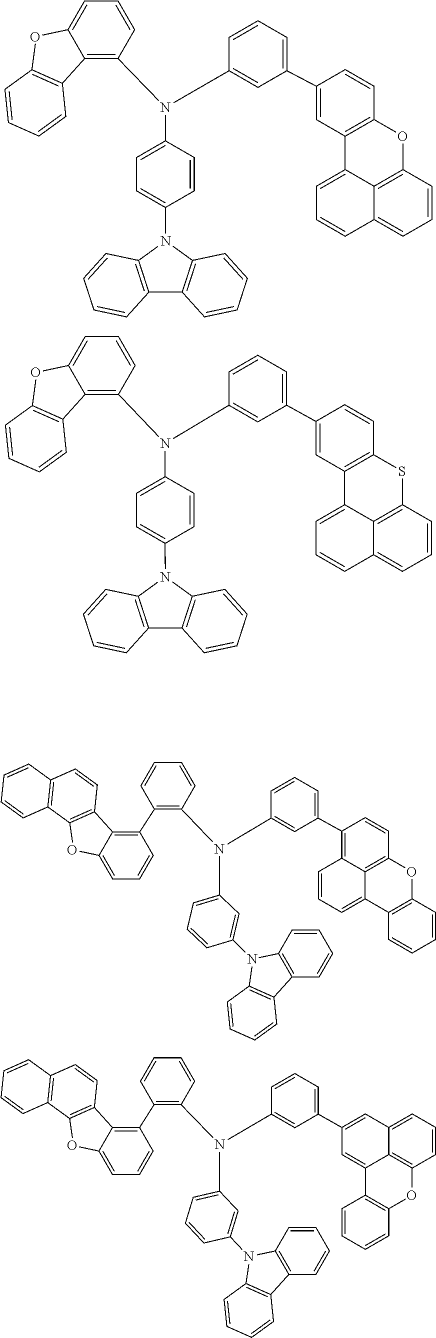

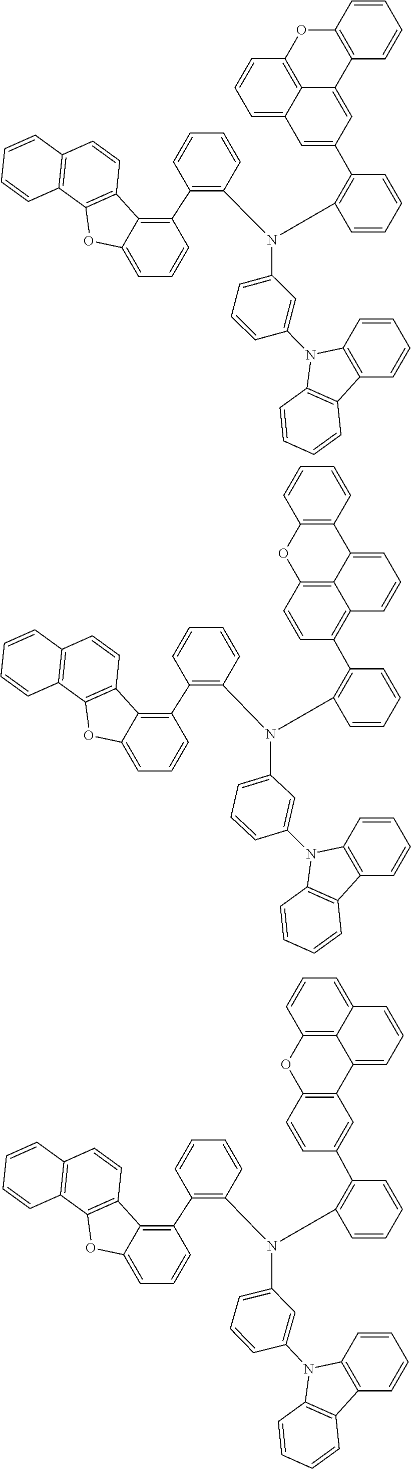

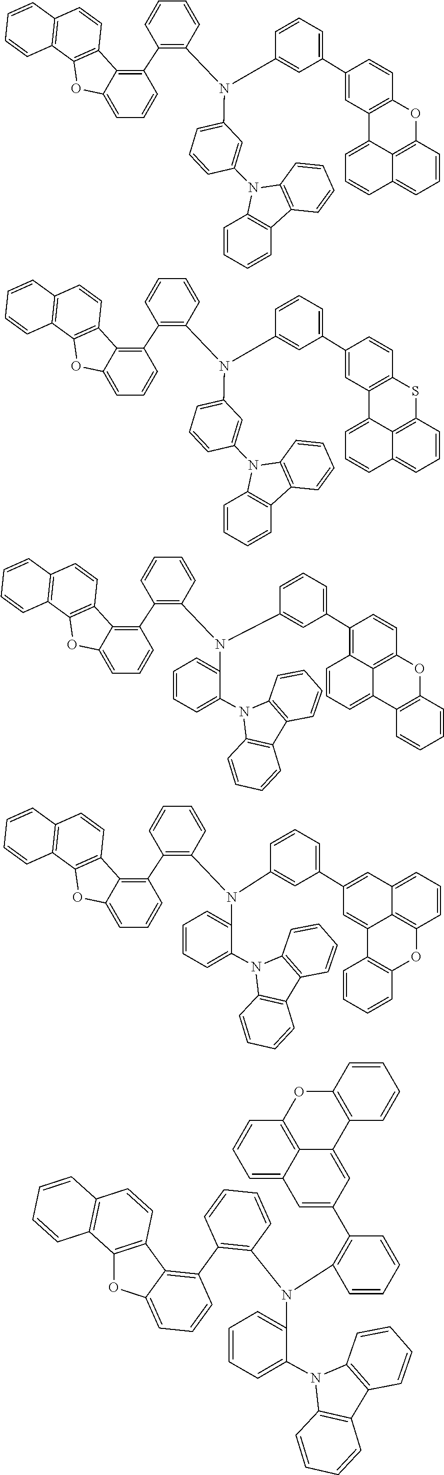

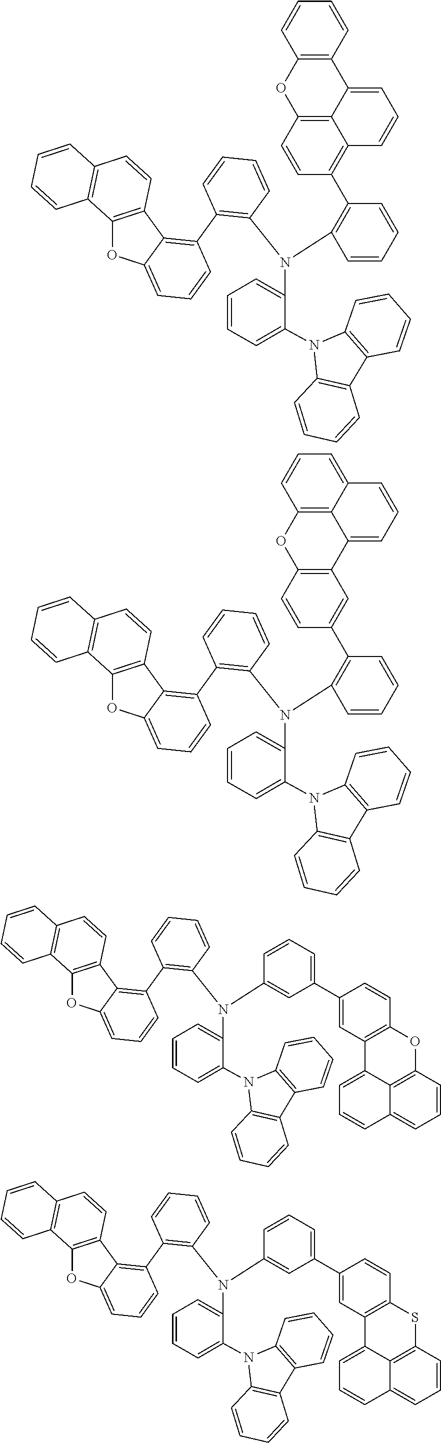

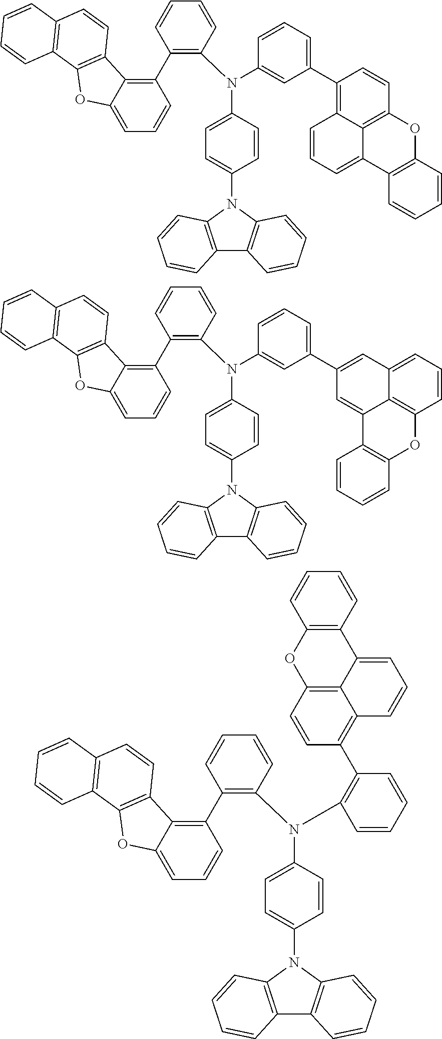

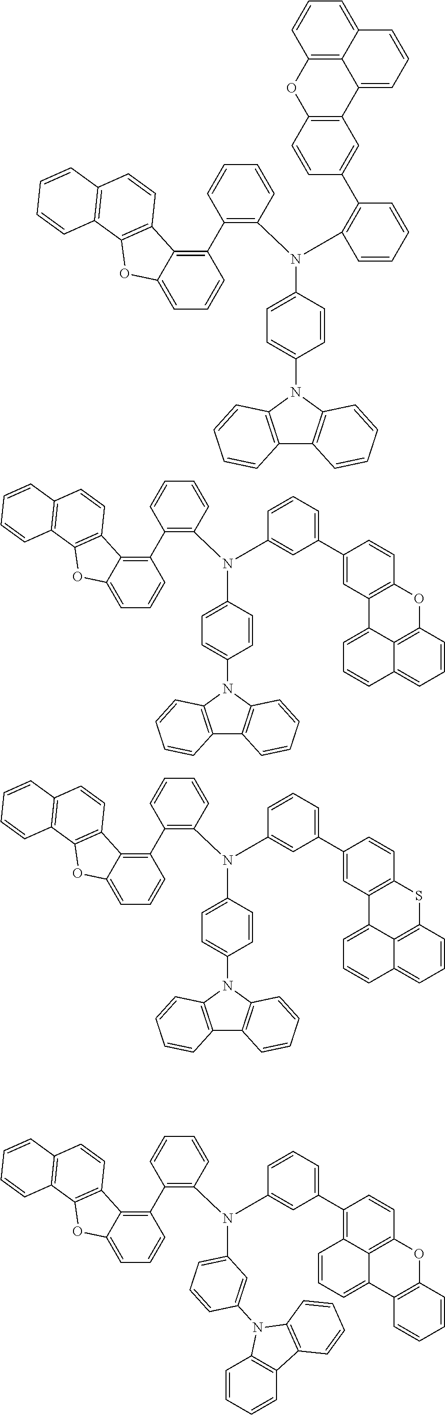

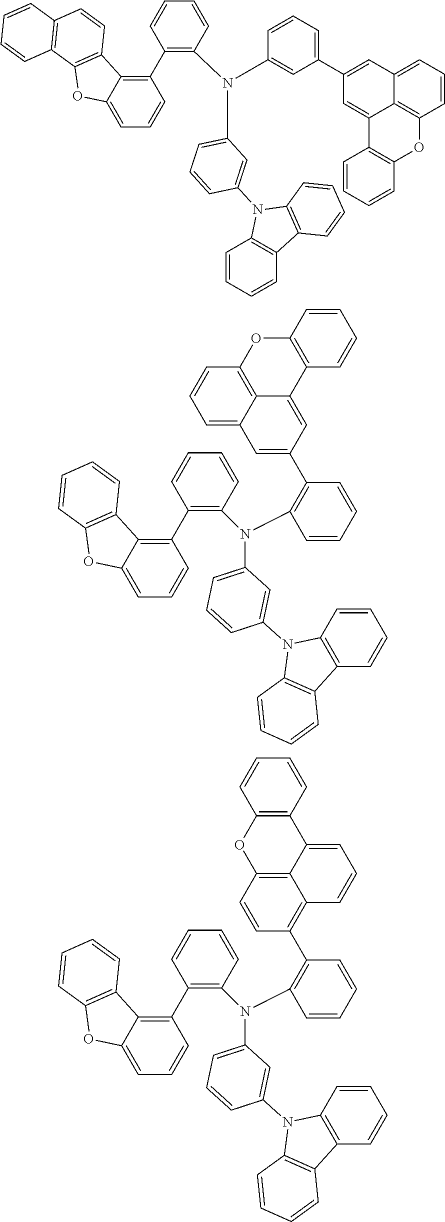

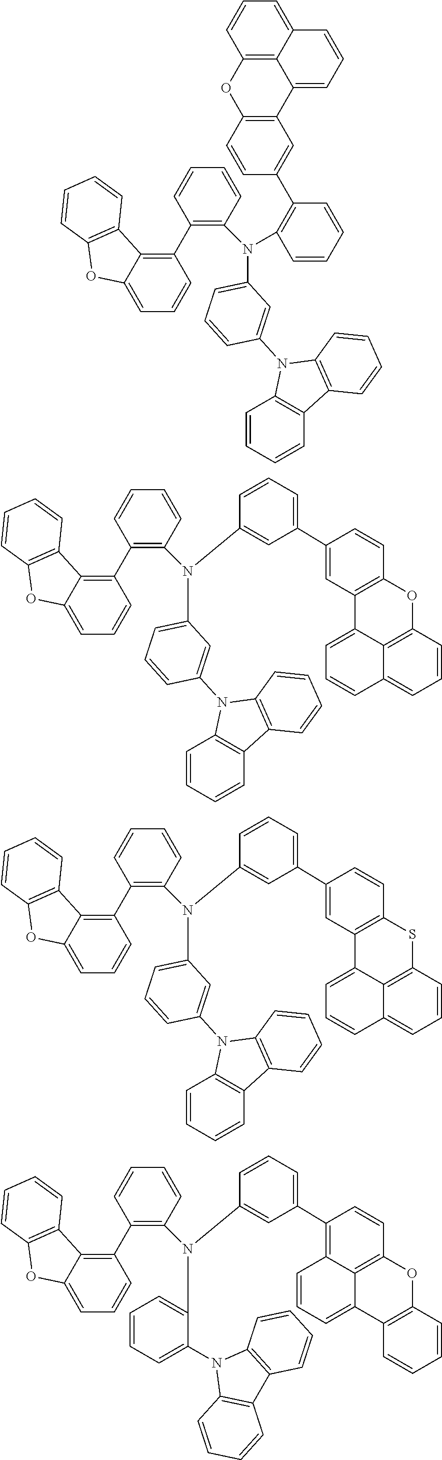

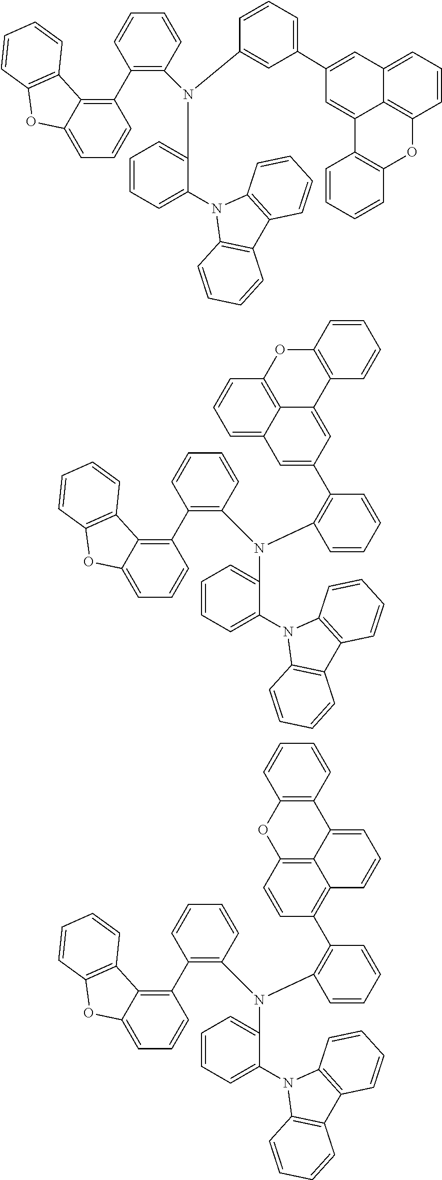

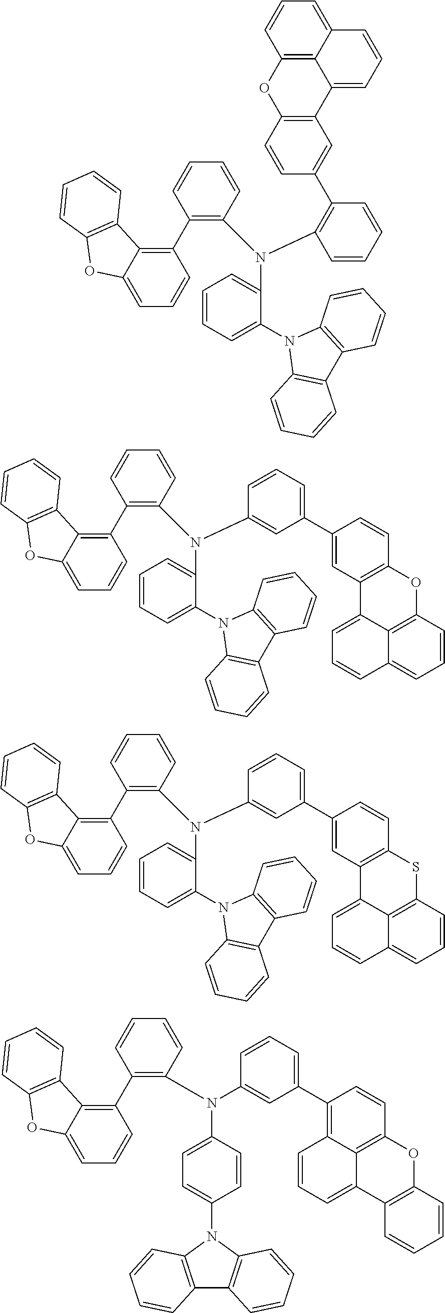

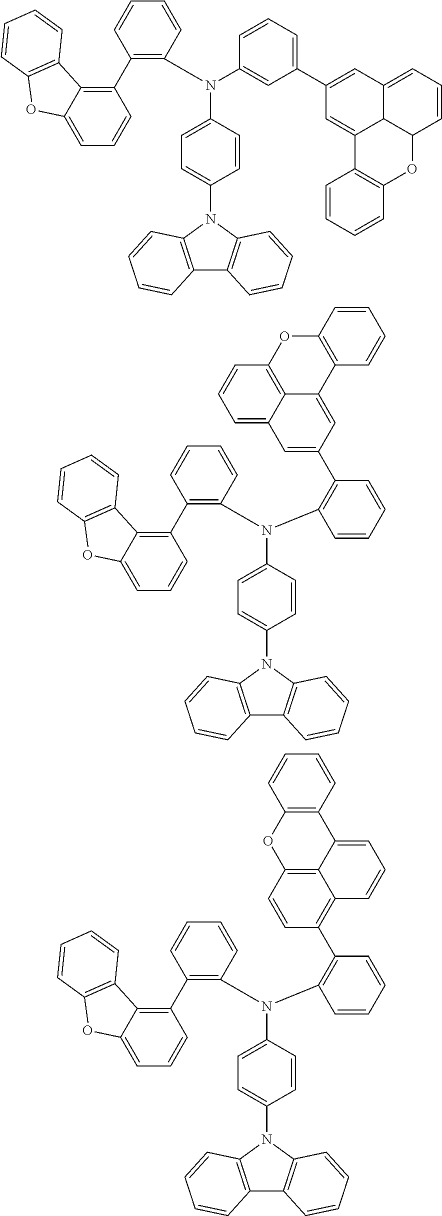

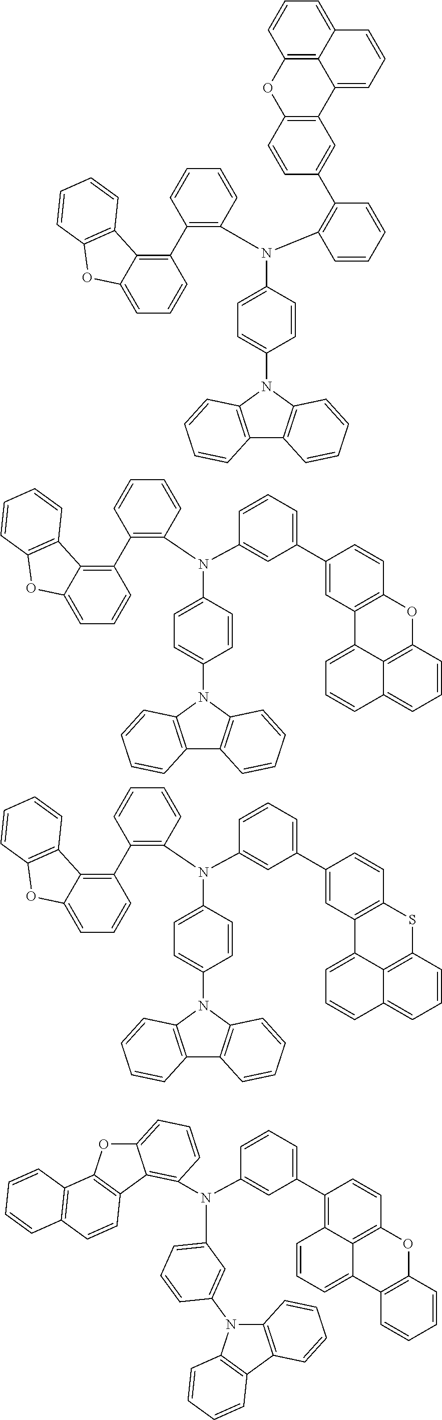

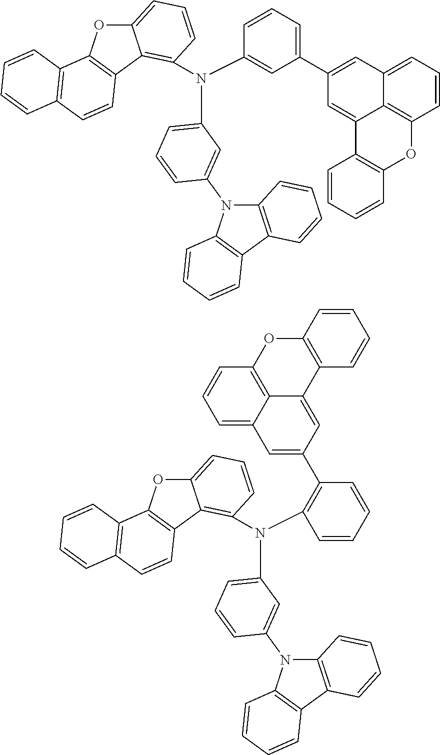 [SMILES: c1cc(-c2cc3c4c(cccc4c2)Oc2ccccc2-3)cc(N(c2cccc(-n3c4ccccc4c4ccccc43)c2)c2cccc3oc4c5ccccc5ccc4c23)c1 ; c1cc(N(c2ccccc2-c2cc3c4c(cccc4c2)Oc2ccccc2-3)c2cccc3oc4c5ccccc5ccc4c23)cc(-n2c3ccccc3c3ccccc32)c1]

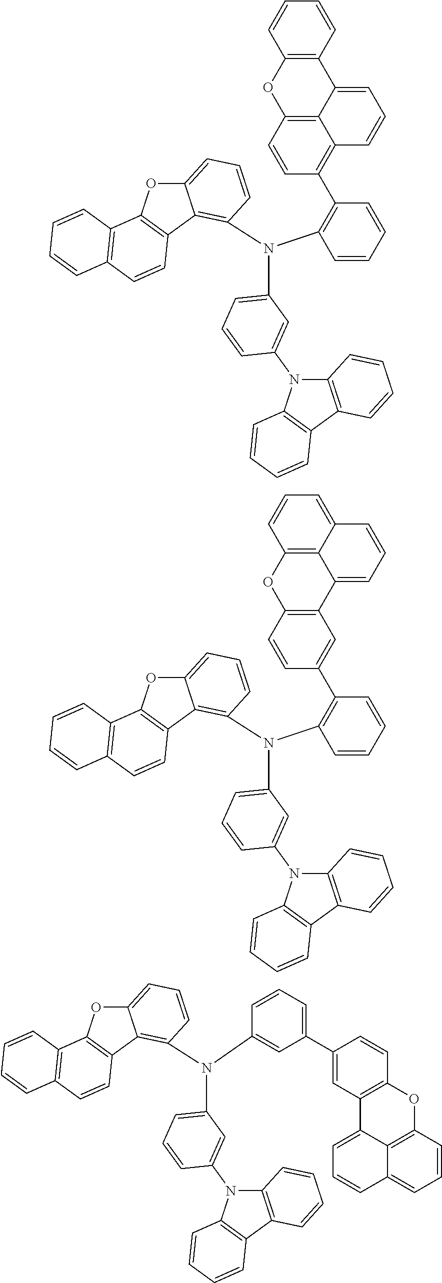

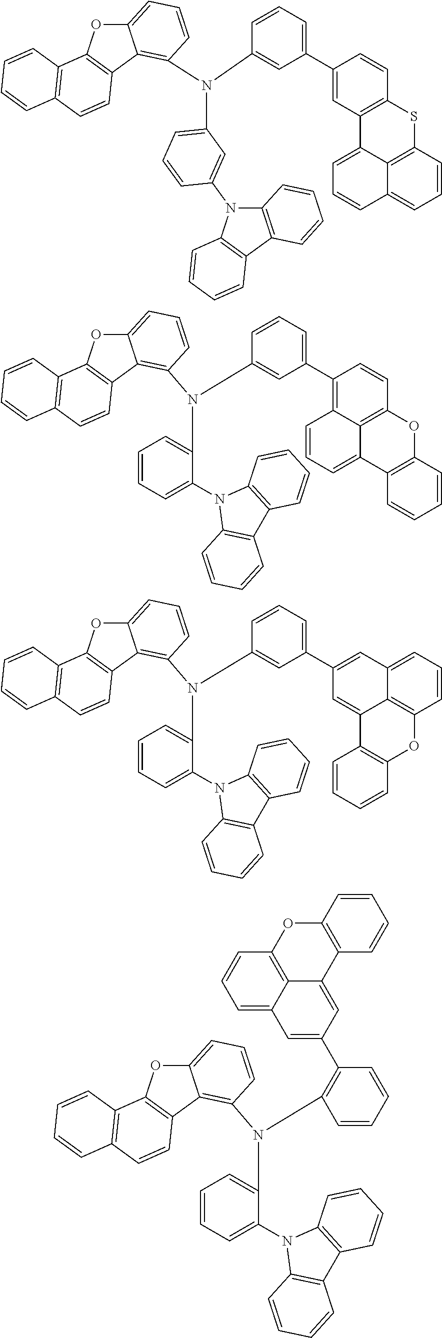

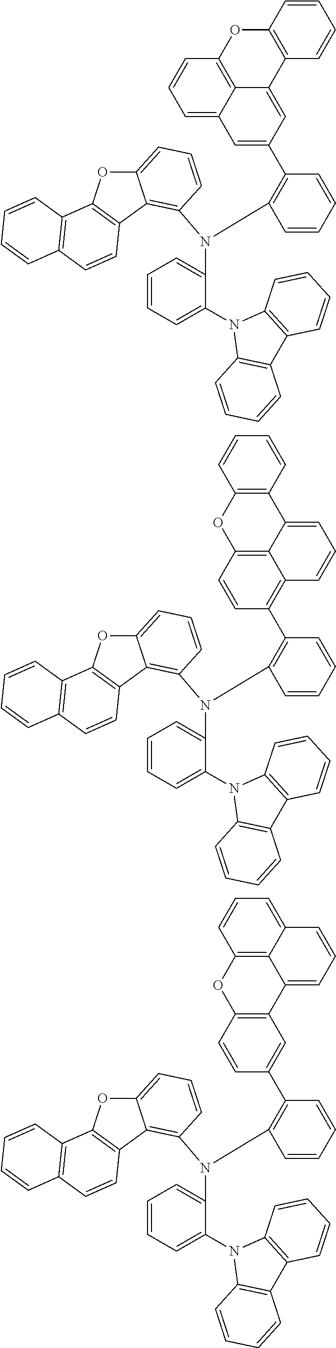

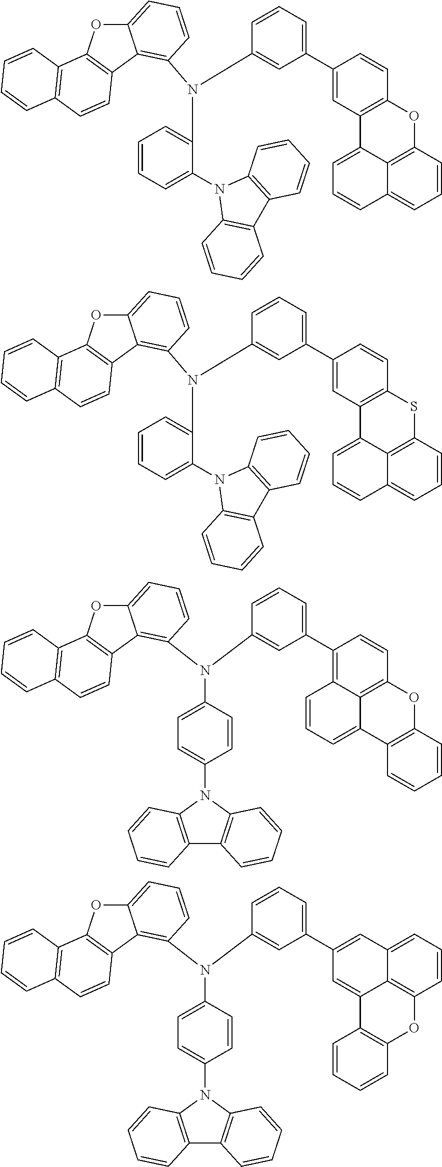

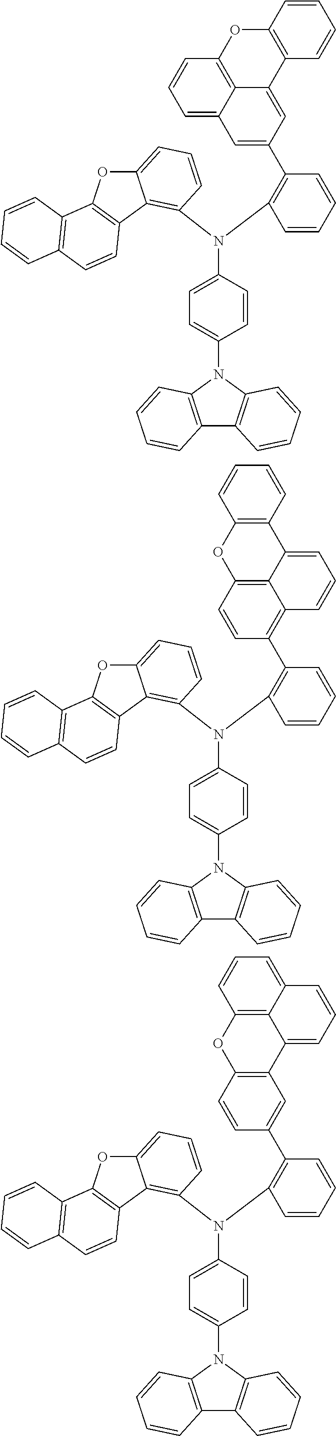

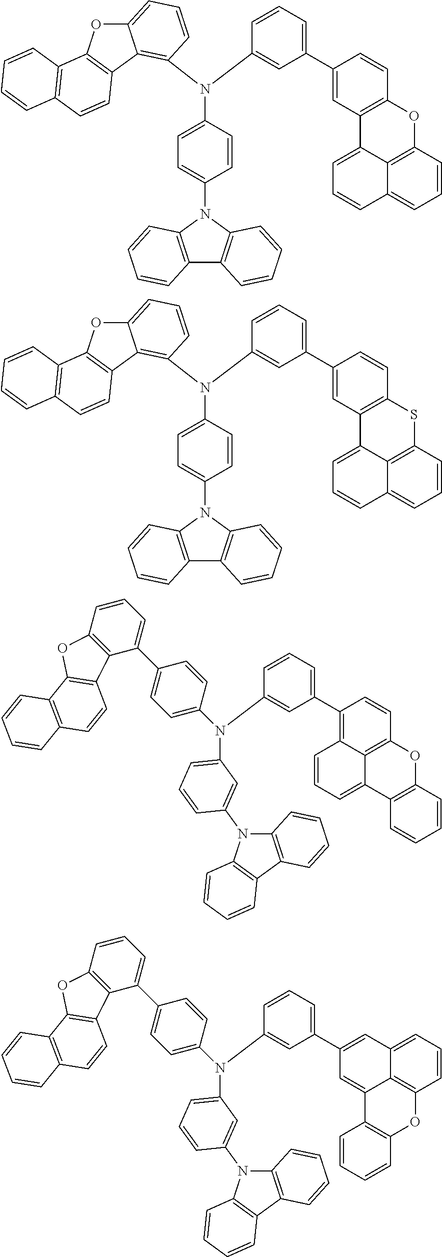

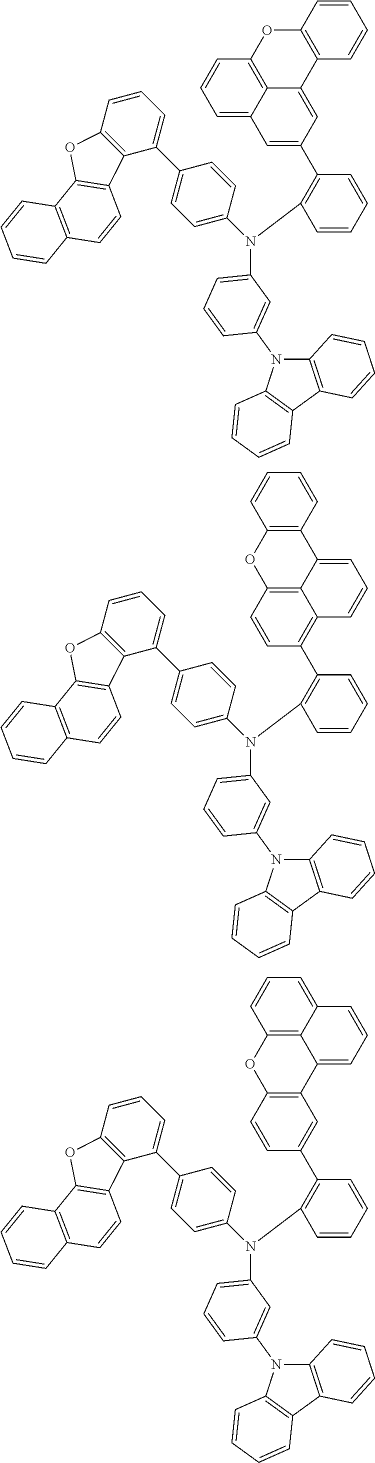

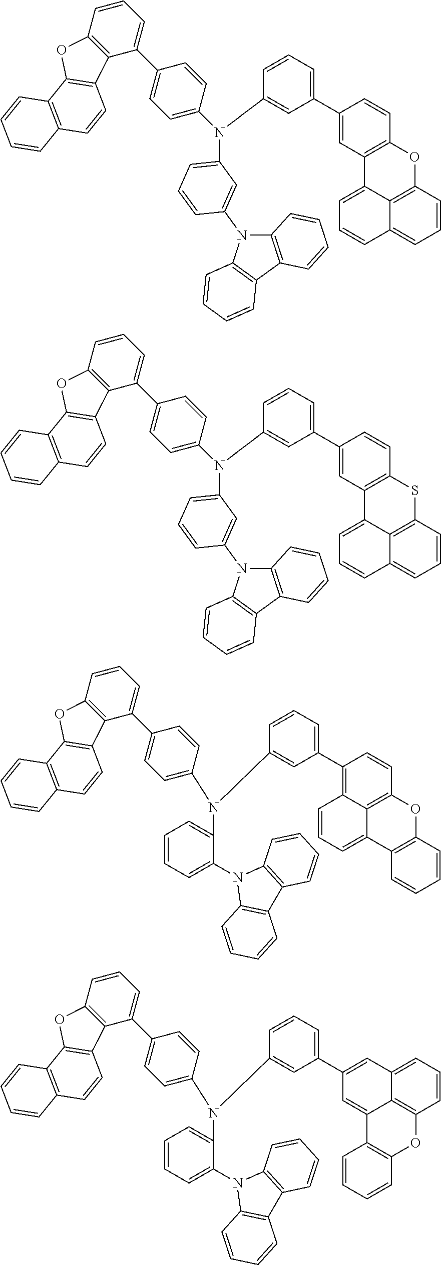 [SMILES: c1cc(-c2ccc3c(c2)-c2cccc4cccc(c24)O3)cc(N(c2ccc(-c3cccc4oc5c6ccccc6ccc5c34)cc2)c2cccc(-n3c4ccccc4c4ccccc43)c2)c1 ; c1cc(-c2ccc3c(c2)-c2cccc4cccc(c24)S3)cc(N(c2ccc(-c3cccc4oc5c6ccccc6ccc5c34)cc2)c2cccc(-n3c4ccccc4c4ccccc43)c2)c1 ; c1cc(-c2ccc3c4c(cccc24)-c2ccccc2O3)cc(N(c2ccc(-c3cccc4oc5c6ccccc6ccc5c34)cc2)c2ccccc2-n2c3ccccc3c3ccccc32)c1 ; c1cc(-c2cc3c4c(cccc4c2)Oc2ccccc2-3)cc(N(c2ccc(-c3cccc4oc5c6ccccc6ccc5c34)cc2)c2ccccc2-n2c3ccccc3c3ccccc32)c1]

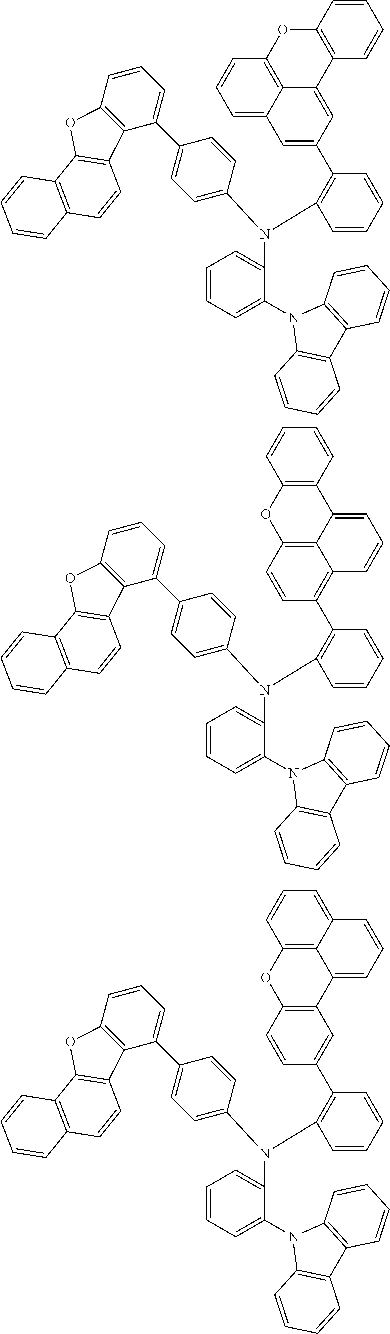

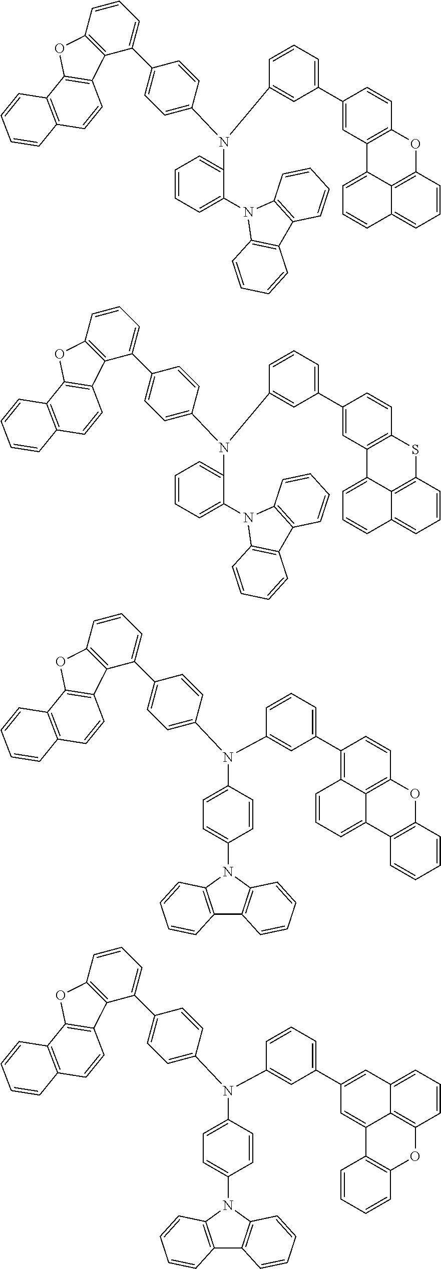 [SMILES: c1cc(-c2ccc3c(c2)-c2cccc4cccc(c24)O3)cc(N(c2ccc(-c3cccc4oc5c6ccccc6ccc5c34)cc2)c2ccccc2-n2c3ccccc3c3ccccc32)c1 ; c1cc(-c2ccc3c(c2)-c2cccc4cccc(c24)S3)cc(N(c2ccc(-c3cccc4oc5c6ccccc6ccc5c34)cc2)c2ccccc2-n2c3ccccc3c3ccccc32)c1 ; c1cc(-c2ccc3c4c(cccc24)-c2ccccc2O3)cc(N(c2ccc(-c3cccc4oc5c6ccccc6ccc5c34)cc2)c2ccc(-n3c4ccccc4c4ccccc43)cc2)c1 ; c1cc(-c2cc3c4c(cccc4c2)Oc2ccccc2-3)cc(N(c2ccc(-c3cccc4oc5c6ccccc6ccc5c34)cc2)c2ccc(-n3c4ccccc4c4ccccc43)cc2)c1]

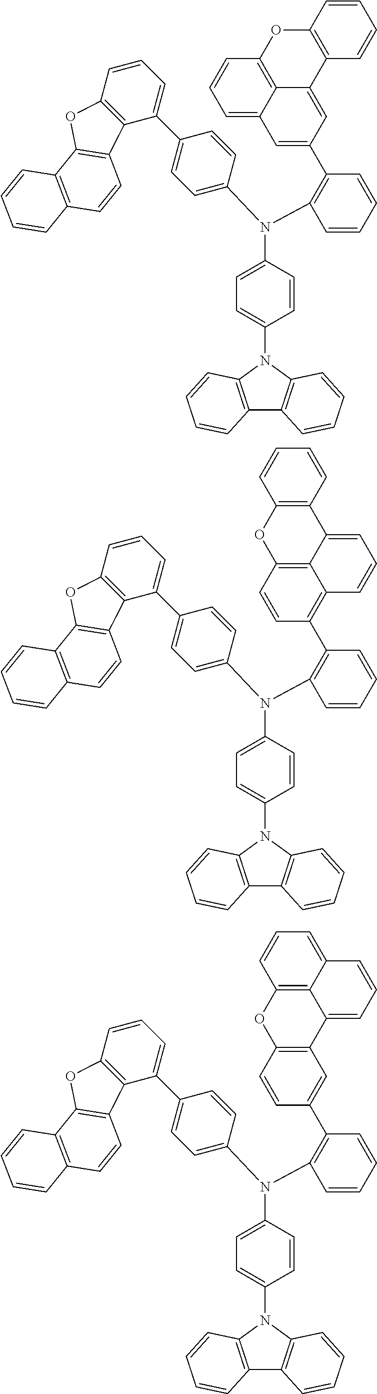

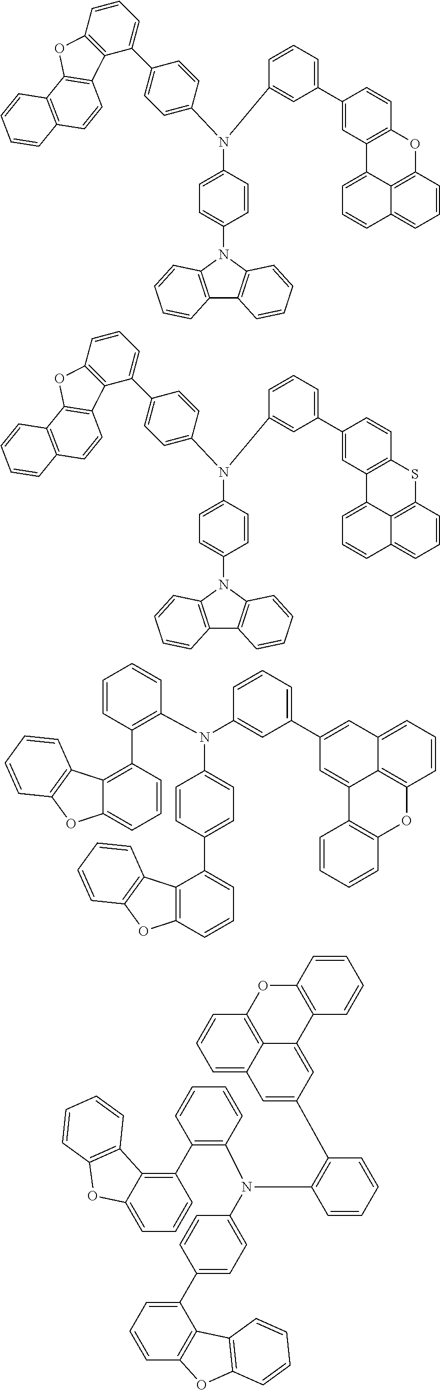

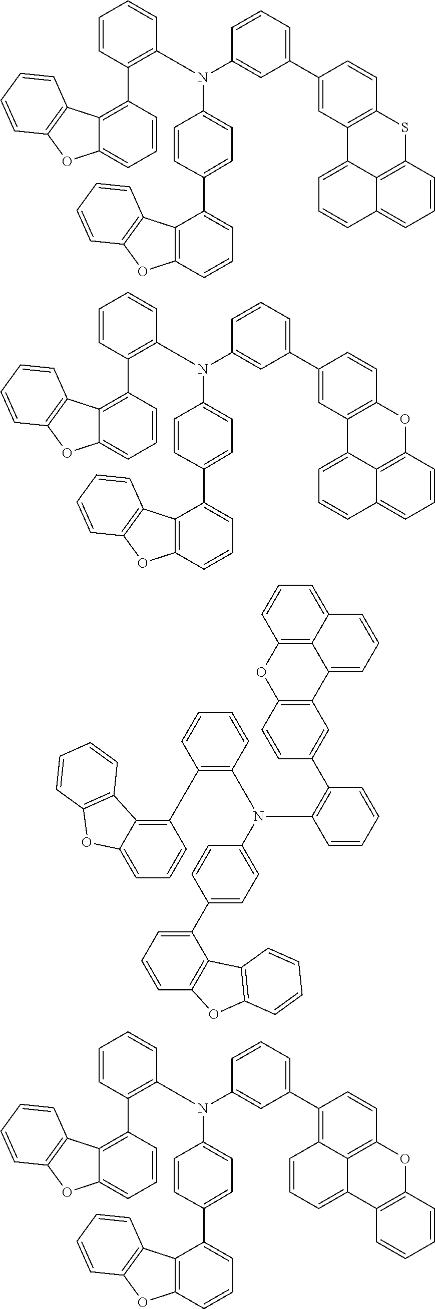

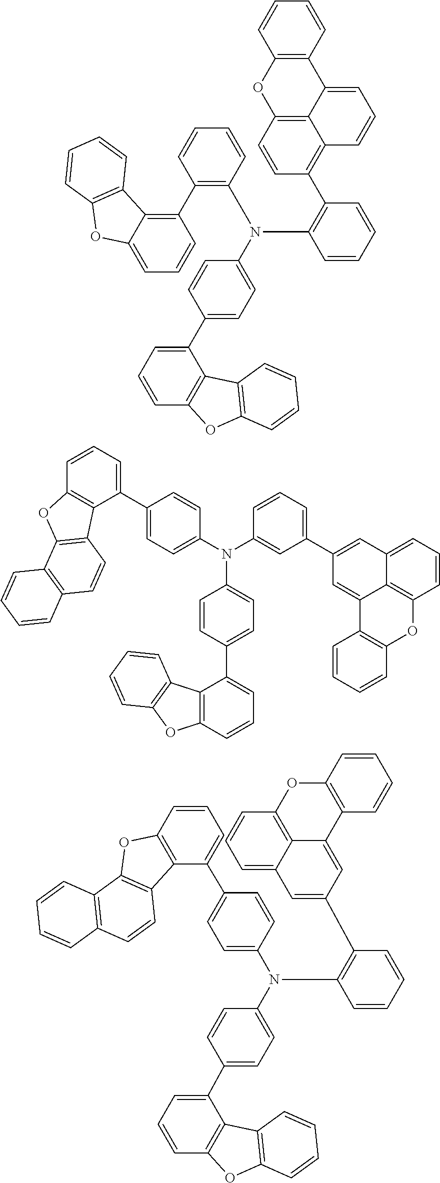 [SMILES: c1ccc2c(c1)Oc1ccc(-c3ccccc3N(c3ccc(-c4cccc5oc6ccccc6c45)cc3)c3ccccc3-c3cccc4oc5ccccc5c34)c3cccc-2c13 ; c1cc(-c2cc3c4c(cccc4c2)Oc2ccccc2-3)cc(N(c2ccc(-c3cccc4oc5ccccc5c34)cc2)c2ccc(-c3cccc4oc5c6ccccc6ccc5c34)cc2)c1 ; c1ccc2c(c1)Oc1cccc3cc(-c4ccccc4N(c4ccc(-c5cccc6oc7ccccc7c56)cc4)c4ccc(-c5cccc6oc7c8ccccc8ccc7c56)cc4)cc-2c13]

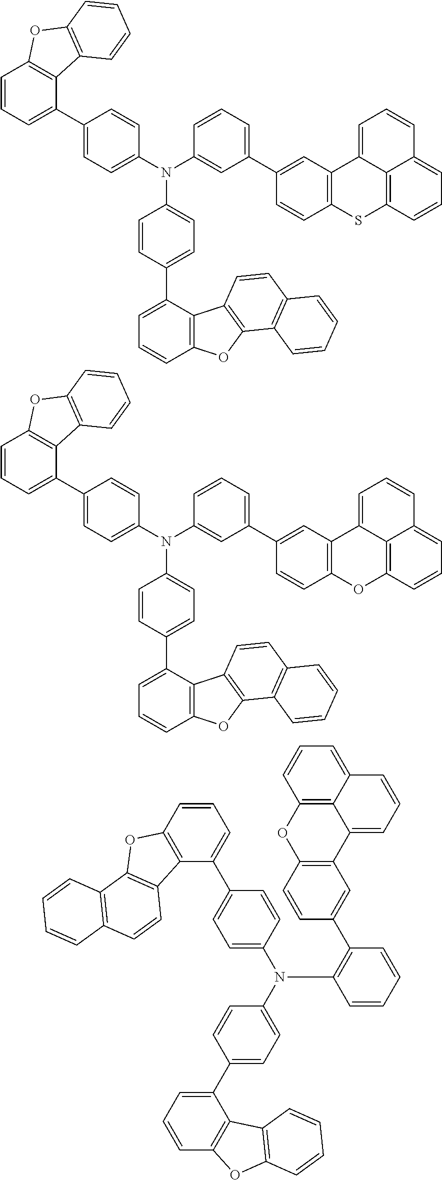

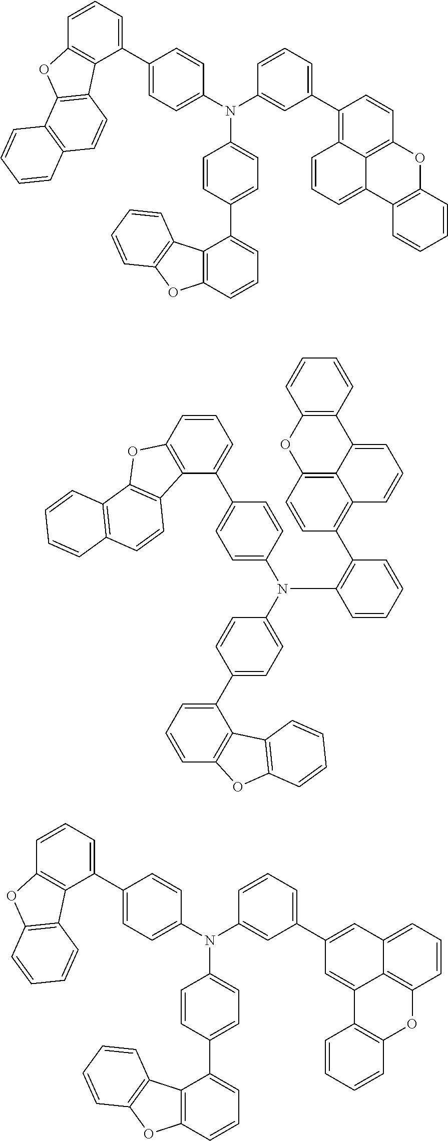 [SMILES: c1cc(-c2ccc3c4c(cccc24)-c2ccccc2O3)cc(N(c2ccc(-c3cccc4oc5ccccc5c34)cc2)c2ccc(-c3cccc4oc5c6ccccc6ccc5c34)cc2)c1 ; c1ccc2c(c1)Oc1ccc(-c3ccccc3N(c3ccc(-c4cccc5oc6ccccc6c45)cc3)c3ccc(-c4cccc5oc6c7ccccc7ccc6c45)cc3)c3cccc-2c13 ; c1cc(-c2cc3c4c(cccc4c2)Oc2ccccc2-3)cc(N(c2ccc(-c3cccc4oc5ccccc5c34)cc2)c2ccc(-c3cccc4oc5ccccc5c34)cc2)c1]

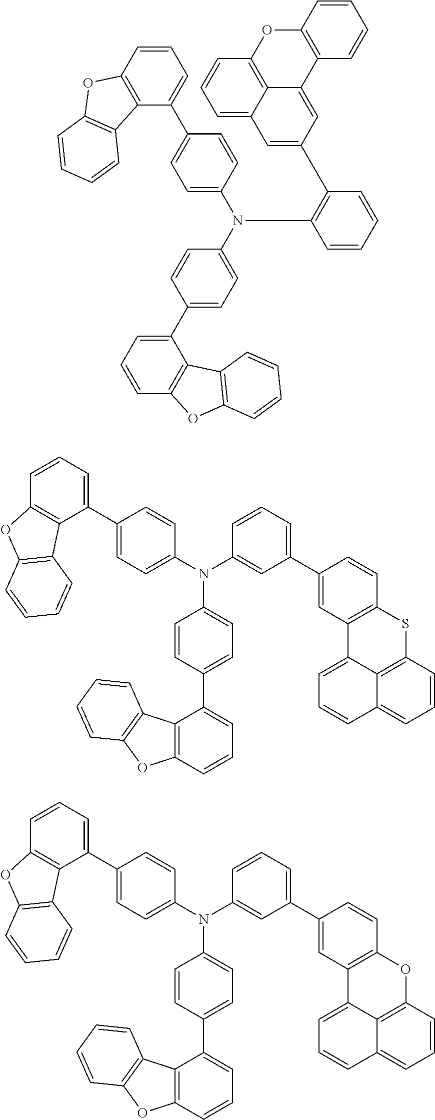 [SMILES: c1ccc2c(c1)Oc1cccc3cc(-c4ccccc4N(c4ccc(-c5cccc6oc7ccccc7c56)cc4)c4ccc(-c5cccc6oc7ccccc7c56)cc4)cc-2c13 ; c1cc(-c2ccc3c(c2)-c2cccc4cccc(c24)S3)cc(N(c2ccc(-c3cccc4oc5ccccc5c34)cc2)c2ccc(-c3cccc4oc5ccccc5c34)cc2)c1 ; c1cc(-c2ccc3c(c2)-c2cccc4cccc(c24)O3)cc(N(c2ccc(-c3cccc4oc5ccccc5c34)cc2)c2ccc(-c3cccc4oc5ccccc5c34)cc2)c1]

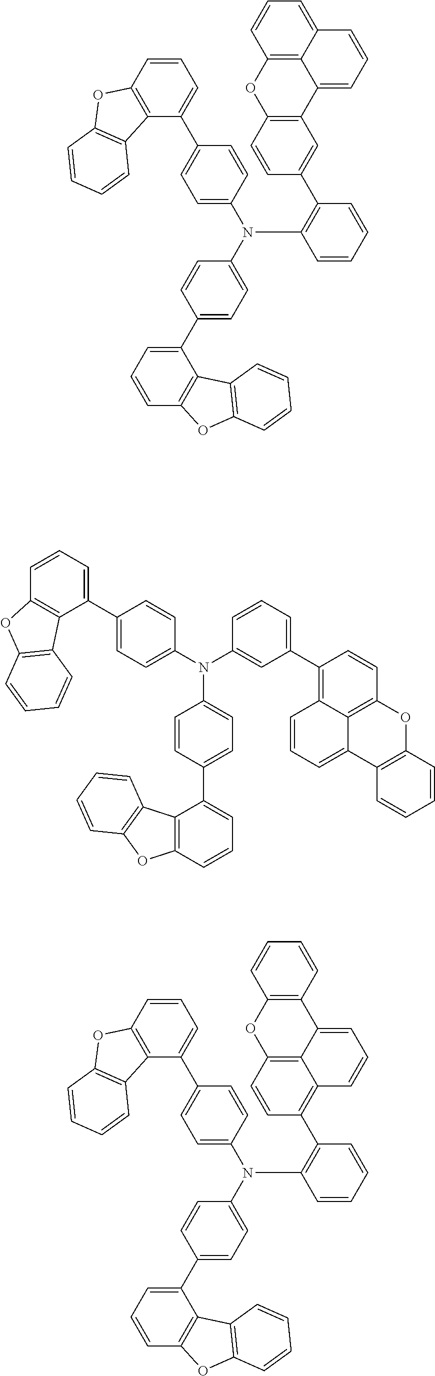 [SMILES: c1ccc(N(c2ccc(-c3cccc4oc5ccccc5c34)cc2)c2ccc(-c3cccc4oc5ccccc5c34)cc2)c(-c2ccc3c(c2)-c2cccc4cccc(c24)O3)c1 ; c1cc(-c2ccc3c4c(cccc24)-c2ccccc2O3)cc(N(c2ccc(-c3cccc4oc5ccccc5c34)cc2)c2ccc(-c3cccc4oc5ccccc5c34)cc2)c1 ; c1ccc2c(c1)Oc1ccc(-c3ccccc3N(c3ccc(-c4cccc5oc6ccccc6c45)cc3)c3ccc(-c4cccc5oc6ccccc6c45)cc3)c3cccc-2c13]

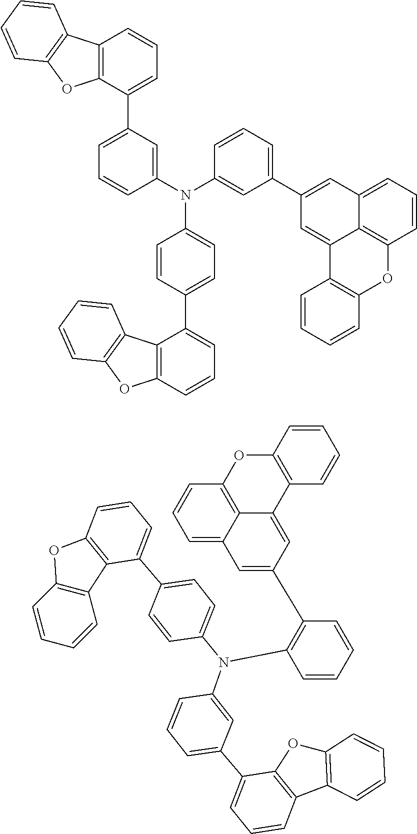 [SMILES: c1cc(-c2cc3c4c(cccc4c2)Oc2ccccc2-3)cc(N(c2ccc(-c3cccc4oc5ccccc5c34)cc2)c2cccc(-c3cccc4c3oc3ccccc34)c2)c1 ; c1cc(-c2cccc3c2oc2ccccc23)cc(N(c2ccc(-c3cccc4oc5ccccc5c34)cc2)c2ccccc2-c2cc3c4c(cccc4c2)Oc2ccccc2-3)c1]

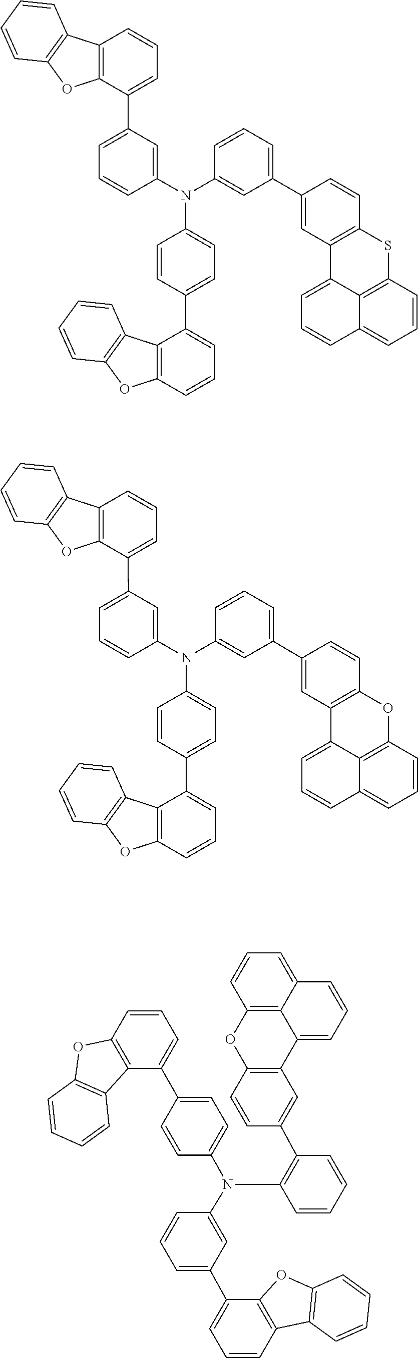 [SMILES: c1cc(-c2ccc3c(c2)-c2cccc4cccc(c24)S3)cc(N(c2ccc(-c3cccc4oc5ccccc5c34)cc2)c2cccc(-c3cccc4c3oc3ccccc34)c2)c1 ; c1cc(-c2ccc3c(c2)-c2cccc4cccc(c24)O3)cc(N(c2ccc(-c3cccc4oc5ccccc5c34)cc2)c2cccc(-c3cccc4c3oc3ccccc34)c2)c1 ; c1cc(-c2cccc3c2oc2ccccc23)cc(N(c2ccc(-c3cccc4oc5ccccc5c34)cc2)c2ccccc2-c2ccc3c(c2)-c2cccc4cccc(c24)O3)c1]

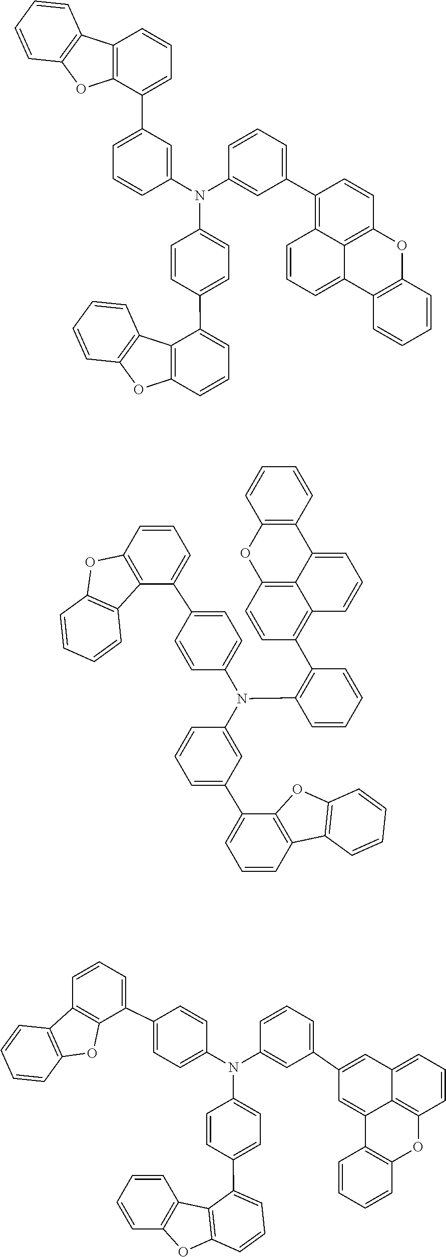 [SMILES: c1cc(-c2ccc3c4c(cccc24)-c2ccccc2O3)cc(N(c2ccc(-c3cccc4oc5ccccc5c34)cc2)c2cccc(-c3cccc4c3oc3ccccc34)c2)c1 ; c1cc(-c2cccc3c2oc2ccccc23)cc(N(c2ccc(-c3cccc4oc5ccccc5c34)cc2)c2ccccc2-c2ccc3c4c(cccc24)-c2ccccc2O3)c1 ; c1cc(-c2cc3c4c(cccc4c2)Oc2ccccc2-3)cc(N(c2ccc(-c3cccc4c3oc3ccccc34)cc2)c2ccc(-c3cccc4oc5ccccc5c34)cc2)c1]

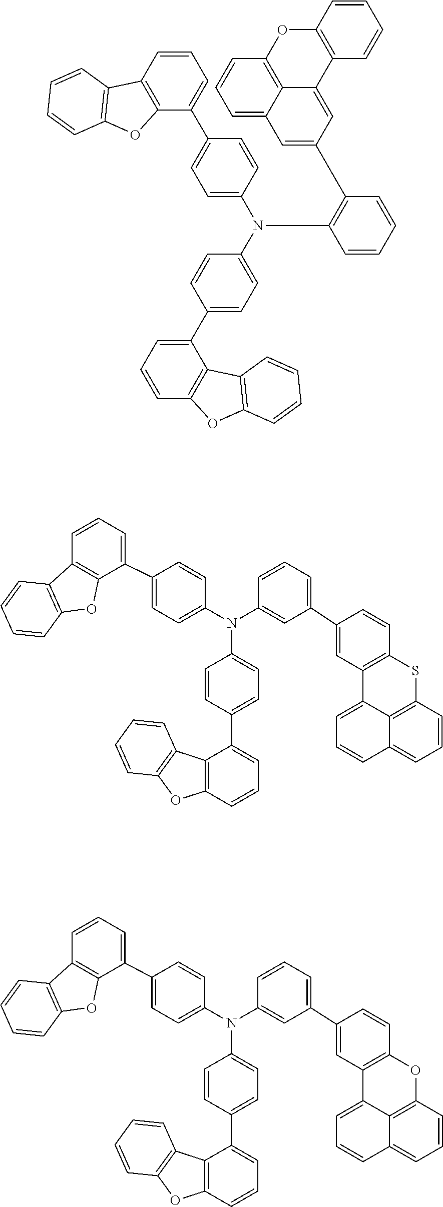 [SMILES: c1ccc2c(c1)Oc1cccc3cc(-c4ccccc4N(c4ccc(-c5cccc6c5oc5ccccc56)cc4)c4ccc(-c5cccc6oc7ccccc7c56)cc4)cc-2c13 ; c1cc(-c2ccc3c(c2)-c2cccc4cccc(c24)S3)cc(N(c2ccc(-c3cccc4c3oc3ccccc34)cc2)c2ccc(-c3cccc4oc5ccccc5c34)cc2)c1 ; c1cc(-c2ccc3c(c2)-c2cccc4cccc(c24)O3)cc(N(c2ccc(-c3cccc4c3oc3ccccc34)cc2)c2ccc(-c3cccc4oc5ccccc5c34)cc2)c1]

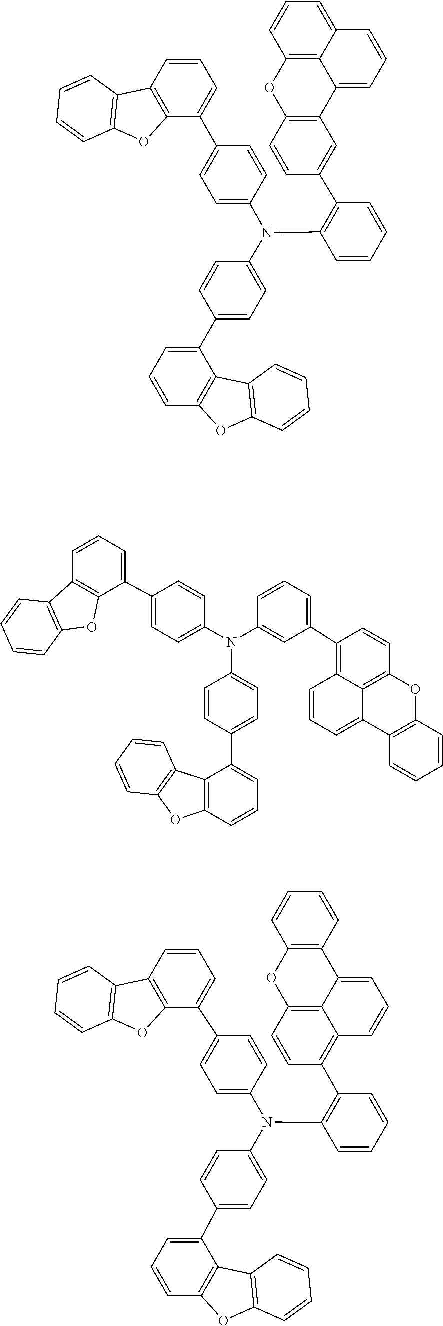 [SMILES: c1ccc(N(c2ccc(-c3cccc4c3oc3ccccc34)cc2)c2ccc(-c3cccc4oc5ccccc5c34)cc2)c(-c2ccc3c(c2)-c2cccc4cccc(c24)O3)c1 ; c1cc(-c2ccc3c4c(cccc24)-c2ccccc2O3)cc(N(c2ccc(-c3cccc4c3oc3ccccc34)cc2)c2ccc(-c3cccc4oc5ccccc5c34)cc2)c1 ; c1ccc2c(c1)Oc1ccc(-c3ccccc3N(c3ccc(-c4cccc5c4oc4ccccc45)cc3)c3ccc(-c4cccc5oc6ccccc6c45)cc3)c3cccc-2c13]

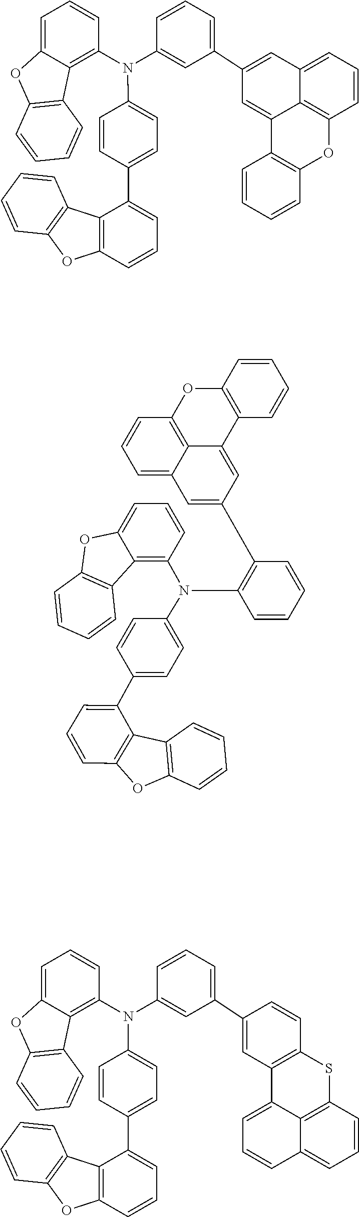 [SMILES: c1cc(-c2cc3c4c(cccc4c2)Oc2ccccc2-3)cc(N(c2ccc(-c3cccc4oc5ccccc5c34)cc2)c2cccc3oc4ccccc4c23)c1 ; c1ccc2c(c1)Oc1cccc3cc(-c4ccccc4N(c4ccc(-c5cccc6oc7ccccc7c56)cc4)c4cccc5oc6ccccc6c45)cc-2c13 ; c1cc(-c2ccc3c(c2)-c2cccc4cccc(c24)S3)cc(N(c2ccc(-c3cccc4oc5ccccc5c34)cc2)c2cccc3oc4ccccc4c23)c1]

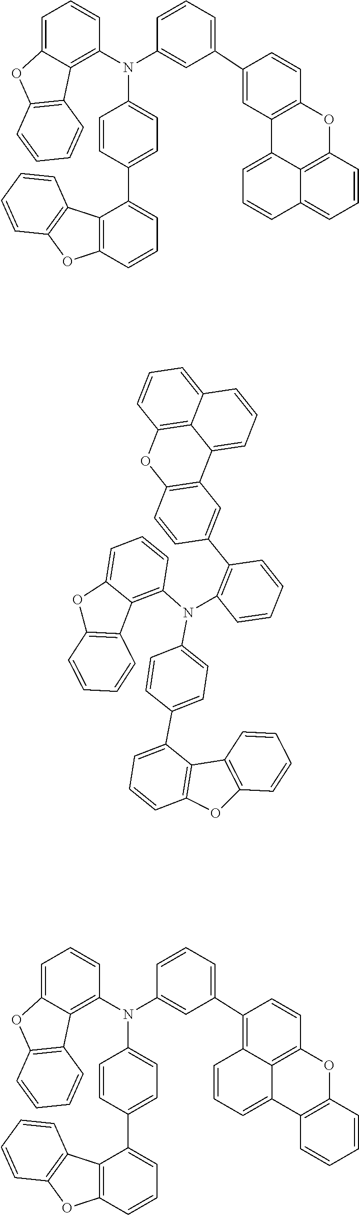 [SMILES: c1cc(-c2ccc3c(c2)-c2cccc4cccc(c24)O3)cc(N(c2ccc(-c3cccc4oc5ccccc5c34)cc2)c2cccc3oc4ccccc4c23)c1 ; c1ccc(N(c2ccc(-c3cccc4oc5ccccc5c34)cc2)c2cccc3oc4ccccc4c23)c(-c2ccc3c(c2)-c2cccc4cccc(c24)O3)c1 ; c1cc(-c2ccc3c4c(cccc24)-c2ccccc2O3)cc(N(c2ccc(-c3cccc4oc5ccccc5c34)cc2)c2cccc3oc4ccccc4c23)c1]

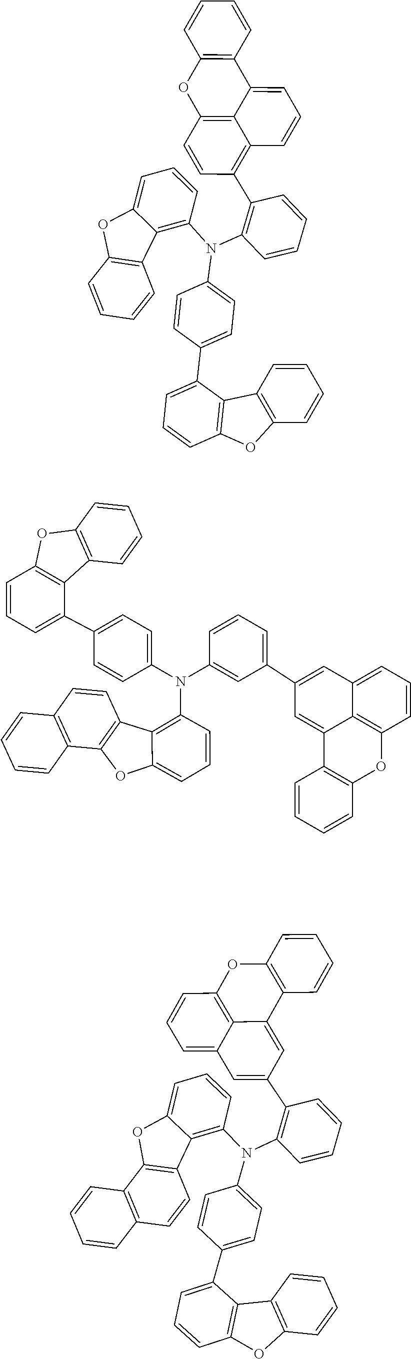 [SMILES: c1ccc2c(c1)Oc1ccc(-c3ccccc3N(c3ccc(-c4cccc5oc6ccccc6c45)cc3)c3cccc4oc5ccccc5c34)c3cccc-2c13 ; c1cc(-c2cc3c4c(cccc4c2)Oc2ccccc2-3)cc(N(c2ccc(-c3cccc4oc5ccccc5c34)cc2)c2cccc3oc4c5ccccc5ccc4c23)c1 ; c1ccc2c(c1)Oc1cccc3cc(-c4ccccc4N(c4ccc(-c5cccc6oc7ccccc7c56)cc4)c4cccc5oc6c7ccccc7ccc6c45)cc-2c13]

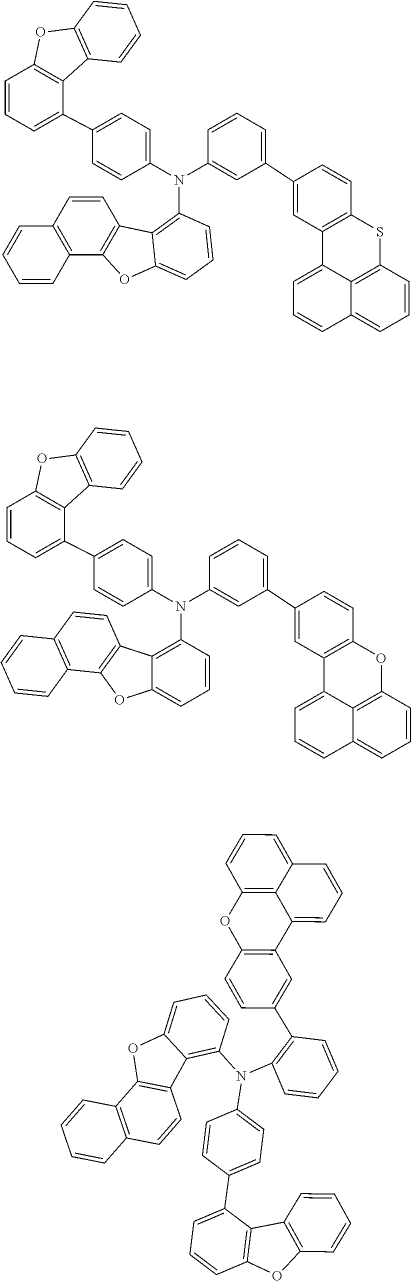 [SMILES: c1cc(-c2ccc3c(c2)-c2cccc4cccc(c24)S3)cc(N(c2ccc(-c3cccc4oc5ccccc5c34)cc2)c2cccc3oc4c5ccccc5ccc4c23)c1 ; c1cc(-c2ccc3c(c2)-c2cccc4cccc(c24)O3)cc(N(c2ccc(-c3cccc4oc5ccccc5c34)cc2)c2cccc3oc4c5ccccc5ccc4c23)c1 ; c1ccc(N(c2ccc(-c3cccc4oc5ccccc5c34)cc2)c2cccc3oc4c5ccccc5ccc4c23)c(-c2ccc3c(c2)-c2cccc4cccc(c24)O3)c1]

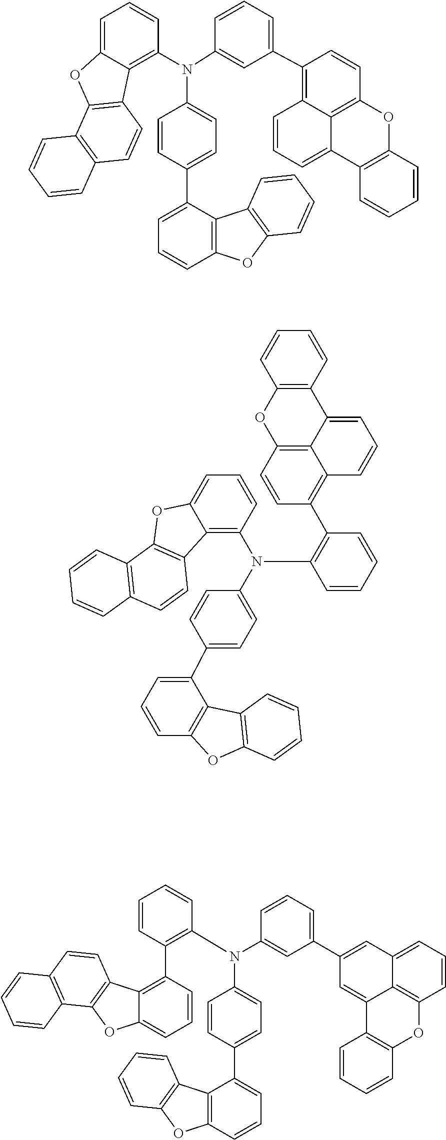 [SMILES: c1cc(-c2ccc3c4c(cccc24)-c2ccccc2O3)cc(N(c2ccc(-c3cccc4oc5ccccc5c34)cc2)c2cccc3oc4c5ccccc5ccc4c23)c1 ; c1ccc2c(c1)Oc1ccc(-c3ccccc3N(c3ccc(-c4cccc5oc6ccccc6c45)cc3)c3cccc4oc5c6ccccc6ccc5c34)c3cccc-2c13 ; c1cc(-c2cc3c4c(cccc4c2)Oc2ccccc2-3)cc(N(c2ccc(-c3cccc4oc5ccccc5c34)cc2)c2ccccc2-c2cccc3oc4c5ccccc5ccc4c23)c1]

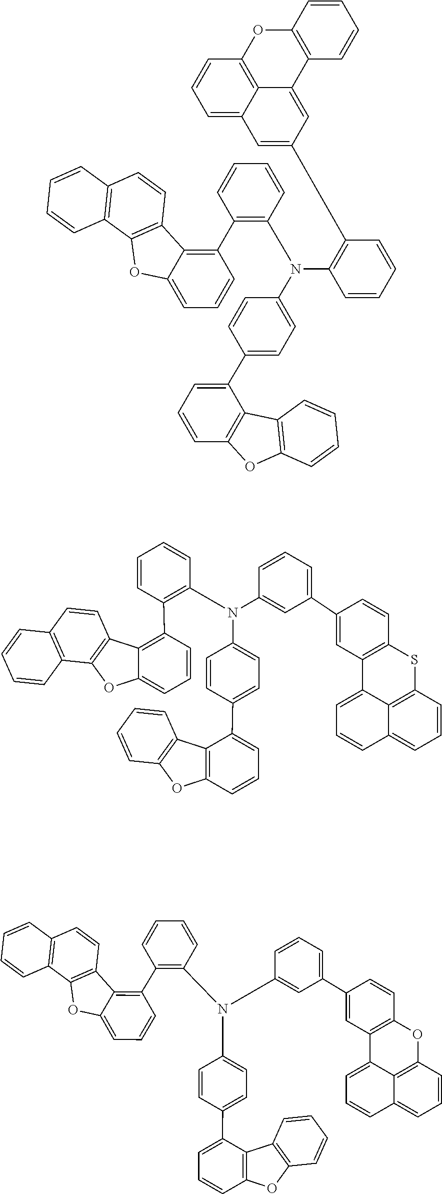 [SMILES: c1ccc2c(c1)Oc1cccc3cc(-c4ccccc4N(c4ccc(-c5cccc6oc7ccccc7c56)cc4)c4ccccc4-c4cccc5oc6c7ccccc7ccc6c45)cc-2c13 ; c1cc(-c2ccc3c(c2)-c2cccc4cccc(c24)S3)cc(N(c2ccc(-c3cccc4oc5ccccc5c34)cc2)c2ccccc2-c2cccc3oc4c5ccccc5ccc4c23)c1 ; c1cc(-c2ccc3c(c2)-c2cccc4cccc(c24)O3)cc(N(c2ccc(-c3cccc4oc5ccccc5c34)cc2)c2ccccc2-c2cccc3oc4c5ccccc5ccc4c23)c1]

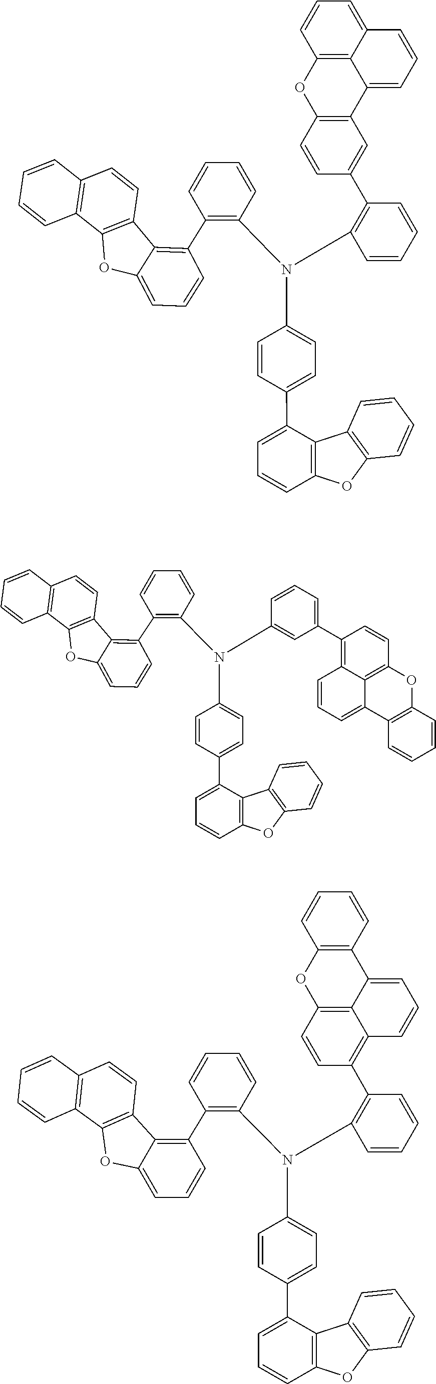 [SMILES: c1ccc(N(c2ccc(-c3cccc4oc5ccccc5c34)cc2)c2ccccc2-c2cccc3oc4c5ccccc5ccc4c23)c(-c2ccc3c(c2)-c2cccc4cccc(c24)O3)c1 ; c1cc(-c2ccc3c4c(cccc24)-c2ccccc2O3)cc(N(c2ccc(-c3cccc4oc5ccccc5c34)cc2)c2ccccc2-c2cccc3oc4c5ccccc5ccc4c23)c1 ; c1ccc2c(c1)Oc1ccc(-c3ccccc3N(c3ccc(-c4cccc5oc6ccccc6c45)cc3)c3ccccc3-c3cccc4oc5c6ccccc6ccc5c34)c3cccc-2c13]

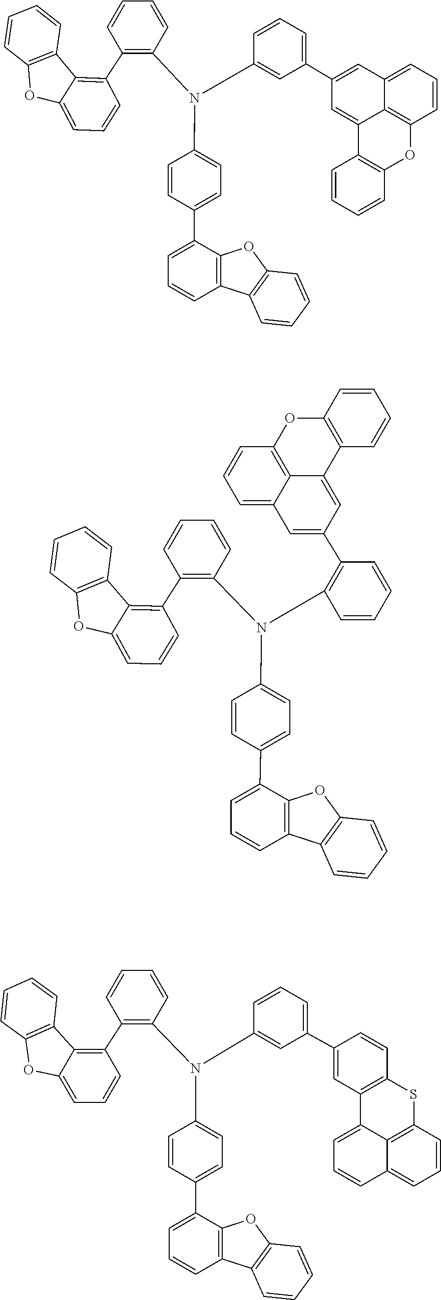 [SMILES: c1cc(-c2cc3c4c(cccc4c2)Oc2ccccc2-3)cc(N(c2ccc(-c3cccc4c3oc3ccccc34)cc2)c2ccccc2-c2cccc3oc4ccccc4c23)c1 ; c1ccc2c(c1)Oc1cccc3cc(-c4ccccc4N(c4ccc(-c5cccc6c5oc5ccccc56)cc4)c4ccccc4-c4cccc5oc6ccccc6c45)cc-2c13 ; c1cc(-c2ccc3c(c2)-c2cccc4cccc(c24)S3)cc(N(c2ccc(-c3cccc4c3oc3ccccc34)cc2)c2ccccc2-c2cccc3oc4ccccc4c23)c1]

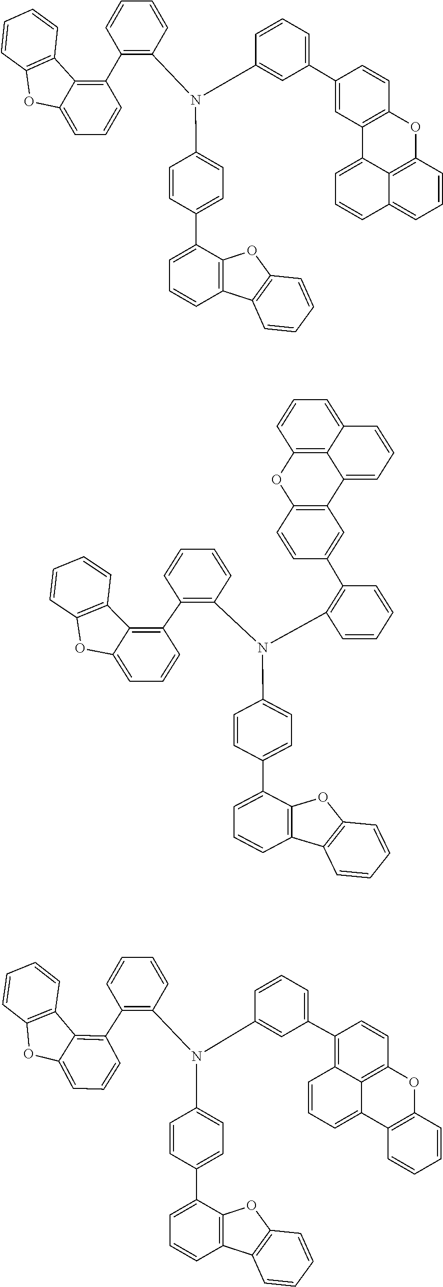 [SMILES: c1cc(-c2ccc3c(c2)-c2cccc4cccc(c24)O3)cc(N(c2ccc(-c3cccc4c3oc3ccccc34)cc2)c2ccccc2-c2cccc3oc4ccccc4c23)c1 ; c1ccc(N(c2ccc(-c3cccc4c3oc3ccccc34)cc2)c2ccccc2-c2cccc3oc4ccccc4c23)c(-c2ccc3c(c2)-c2cccc4cccc(c24)O3)c1 ; c1cc(-c2ccc3c4c(cccc24)-c2ccccc2O3)cc(N(c2ccc(-c3cccc4c3oc3ccccc34)cc2)c2ccccc2-c2cccc3oc4ccccc4c23)c1]

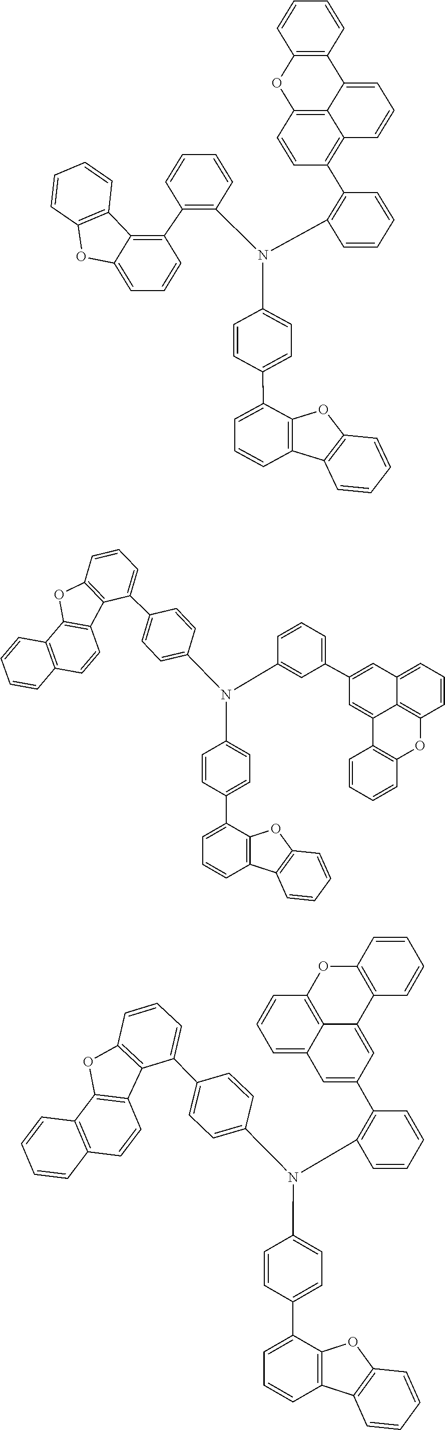 [SMILES: c1ccc2c(c1)Oc1ccc(-c3ccccc3N(c3ccc(-c4cccc5c4oc4ccccc45)cc3)c3ccccc3-c3cccc4oc5ccccc5c34)c3cccc-2c13 ; c1cc(-c2cc3c4c(cccc4c2)Oc2ccccc2-3)cc(N(c2ccc(-c3cccc4c3oc3ccccc34)cc2)c2ccc(-c3cccc4oc5c6ccccc6ccc5c34)cc2)c1 ; c1ccc2c(c1)Oc1cccc3cc(-c4ccccc4N(c4ccc(-c5cccc6c5oc5ccccc56)cc4)c4ccc(-c5cccc6oc7c8ccccc8ccc7c56)cc4)cc-2c13]

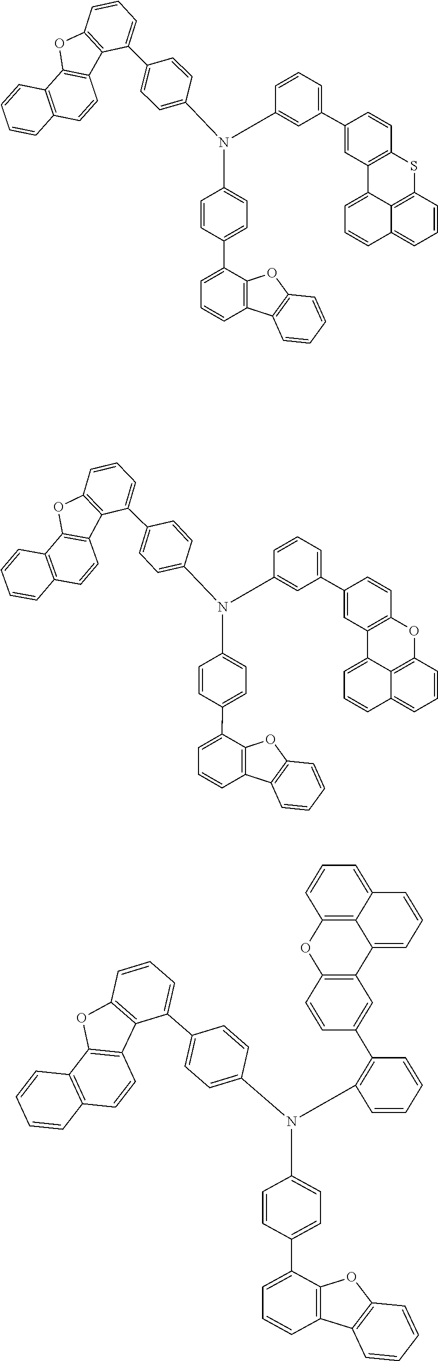 [SMILES: c1cc(-c2ccc3c(c2)-c2cccc4cccc(c24)S3)cc(N(c2ccc(-c3cccc4c3oc3ccccc34)cc2)c2ccc(-c3cccc4oc5c6ccccc6ccc5c34)cc2)c1 ; c1cc(-c2ccc3c(c2)-c2cccc4cccc(c24)O3)cc(N(c2ccc(-c3cccc4c3oc3ccccc34)cc2)c2ccc(-c3cccc4oc5c6ccccc6ccc5c34)cc2)c1 ; c1ccc(N(c2ccc(-c3cccc4c3oc3ccccc34)cc2)c2ccc(-c3cccc4oc5c6ccccc6ccc5c34)cc2)c(-c2ccc3c(c2)-c2cccc4cccc(c24)O3)c1]

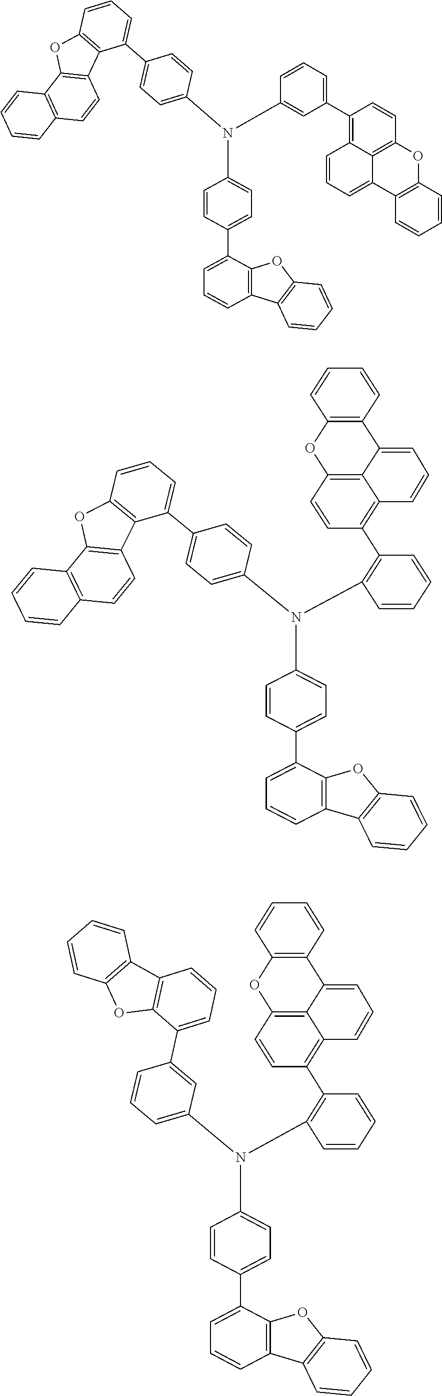

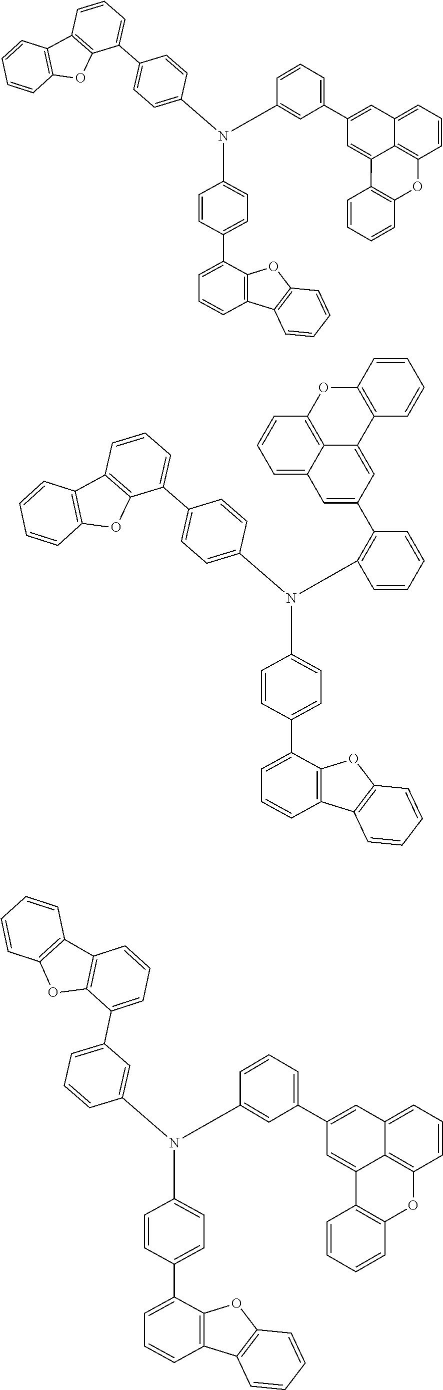 [SMILES: c1cc(-c2cc3c4c(cccc4c2)Oc2ccccc2-3)cc(N(c2ccc(-c3cccc4c3oc3ccccc34)cc2)c2ccc(-c3cccc4c3oc3ccccc34)cc2)c1 ; c1ccc2c(c1)Oc1cccc3cc(-c4ccccc4N(c4ccc(-c5cccc6c5oc5ccccc56)cc4)c4ccc(-c5cccc6c5oc5ccccc56)cc4)cc-2c13 ; c1cc(-c2cc3c4c(cccc4c2)Oc2ccccc2-3)cc(N(c2ccc(-c3cccc4c3oc3ccccc34)cc2)c2cccc(-c3cccc4c3oc3ccccc34)c2)c1]

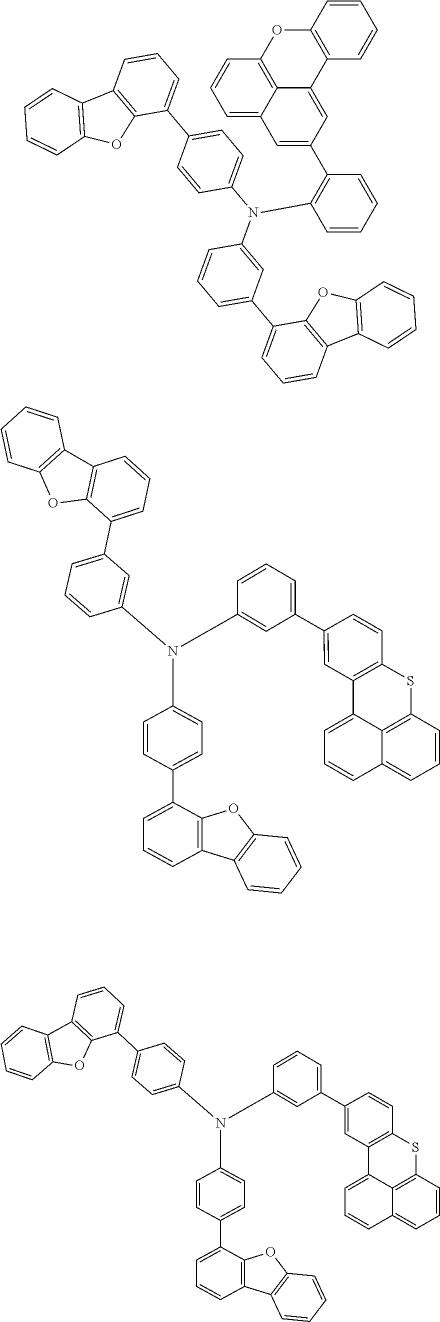 [SMILES: c1cc(-c2cccc3c2oc2ccccc23)cc(N(c2ccc(-c3cccc4c3oc3ccccc34)cc2)c2ccccc2-c2cc3c4c(cccc4c2)Oc2ccccc2-3)c1 ; c1cc(-c2ccc3c(c2)-c2cccc4cccc(c24)S3)cc(N(c2ccc(-c3cccc4c3oc3ccccc34)cc2)c2cccc(-c3cccc4c3oc3ccccc34)c2)c1 ; c1cc(-c2ccc3c(c2)-c2cccc4cccc(c24)S3)cc(N(c2ccc(-c3cccc4c3oc3ccccc34)cc2)c2ccc(-c3cccc4c3oc3ccccc34)cc2)c1]

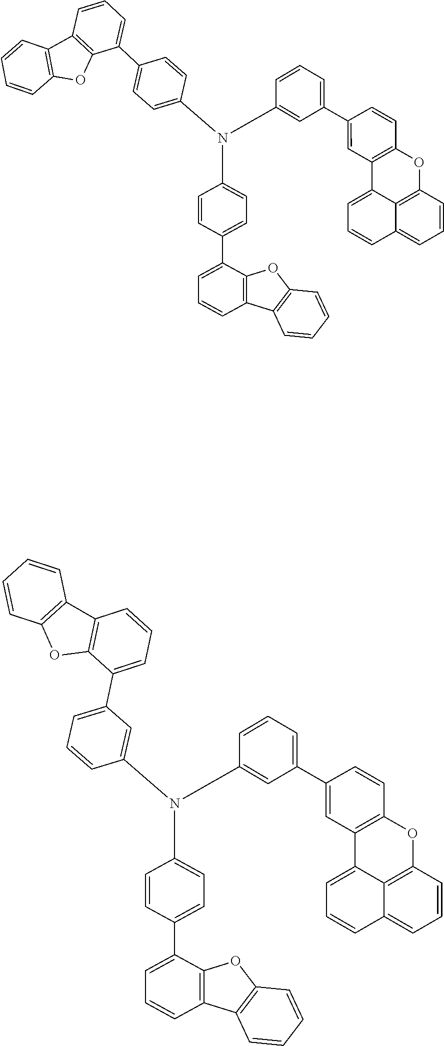 [SMILES: c1cc(-c2ccc3c(c2)-c2cccc4cccc(c24)O3)cc(N(c2ccc(-c3cccc4c3oc3ccccc34)cc2)c2ccc(-c3cccc4c3oc3ccccc34)cc2)c1 ; c1cc(-c2ccc3c(c2)-c2cccc4cccc(c24)O3)cc(N(c2ccc(-c3cccc4c3oc3ccccc34)cc2)c2cccc(-c3cccc4c3oc3ccccc34)c2)c1]

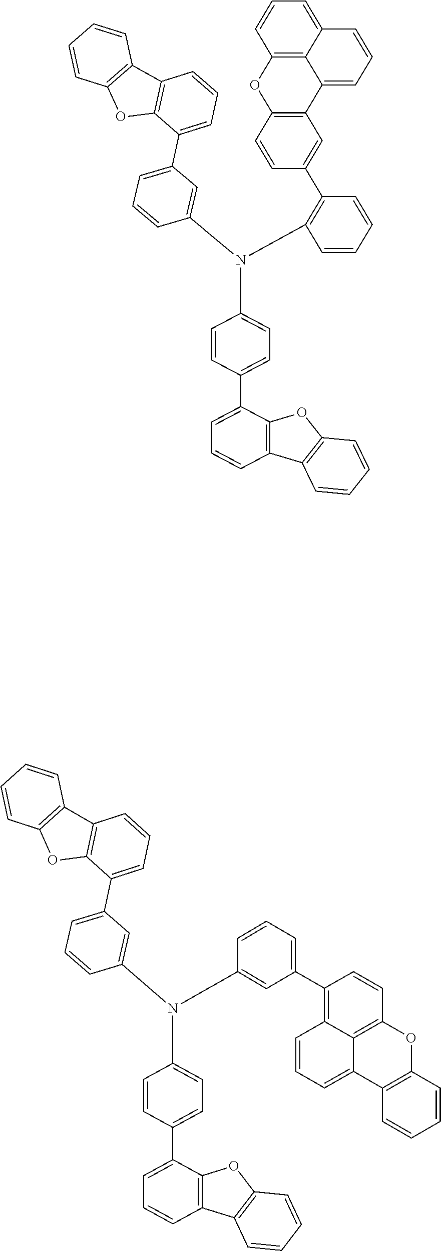 [SMILES: c1cc(-c2cccc3c2oc2ccccc23)cc(N(c2ccc(-c3cccc4c3oc3ccccc34)cc2)c2ccccc2-c2ccc3c(c2)-c2cccc4cccc(c24)O3)c1 ; c1cc(-c2ccc3c4c(cccc24)-c2ccccc2O3)cc(N(c2ccc(-c3cccc4c3oc3ccccc34)cc2)c2cccc(-c3cccc4c3oc3ccccc34)c2)c1]

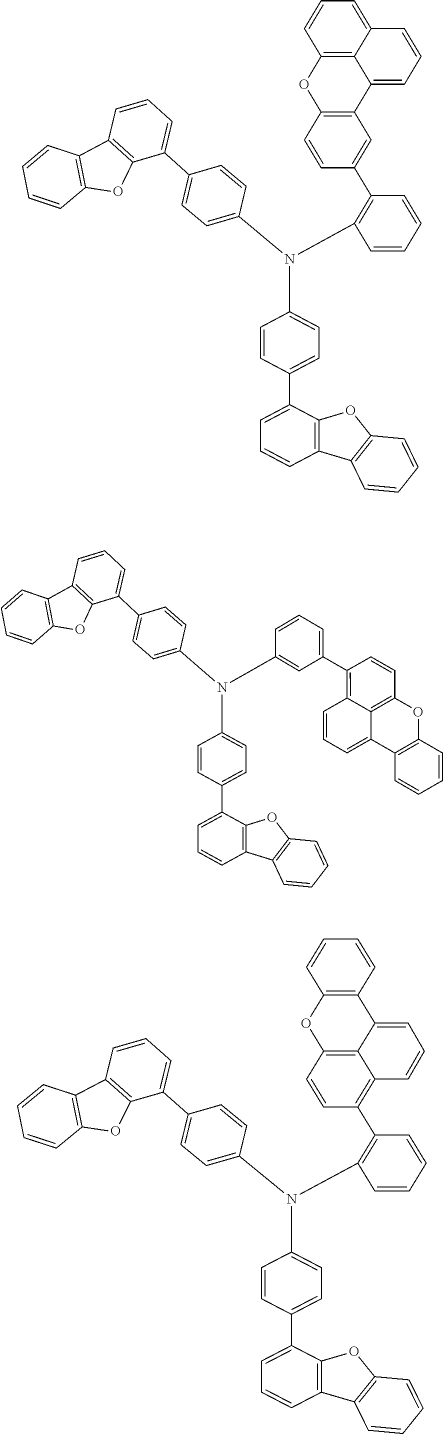 [SMILES: c1ccc(N(c2ccc(-c3cccc4c3oc3ccccc34)cc2)c2ccc(-c3cccc4c3oc3ccccc34)cc2)c(-c2ccc3c(c2)-c2cccc4cccc(c24)O3)c1 ; c1cc(-c2ccc3c4c(cccc24)-c2ccccc2O3)cc(N(c2ccc(-c3cccc4c3oc3ccccc34)cc2)c2ccc(-c3cccc4c3oc3ccccc34)cc2)c1 ; c1ccc2c(c1)Oc1ccc(-c3ccccc3N(c3ccc(-c4cccc5c4oc4ccccc45)cc3)c3ccc(-c4cccc5c4oc4ccccc45)cc3)c3cccc-2c13]

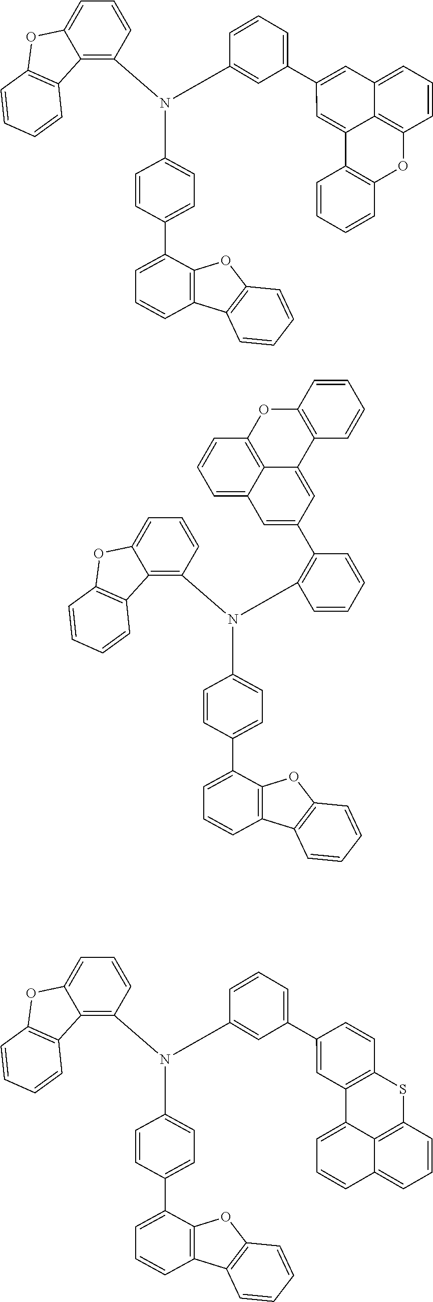 [SMILES: c1cc(-c2cc3c4c(cccc4c2)Oc2ccccc2-3)cc(N(c2ccc(-c3cccc4c3oc3ccccc34)cc2)c2cccc3oc4ccccc4c23)c1 ; c1ccc2c(c1)Oc1cccc3cc(-c4ccccc4N(c4ccc(-c5cccc6c5oc5ccccc56)cc4)c4cccc5oc6ccccc6c45)cc-2c13 ; c1cc(-c2ccc3c(c2)-c2cccc4cccc(c24)S3)cc(N(c2ccc(-c3cccc4c3oc3ccccc34)cc2)c2cccc3oc4ccccc4c23)c1]

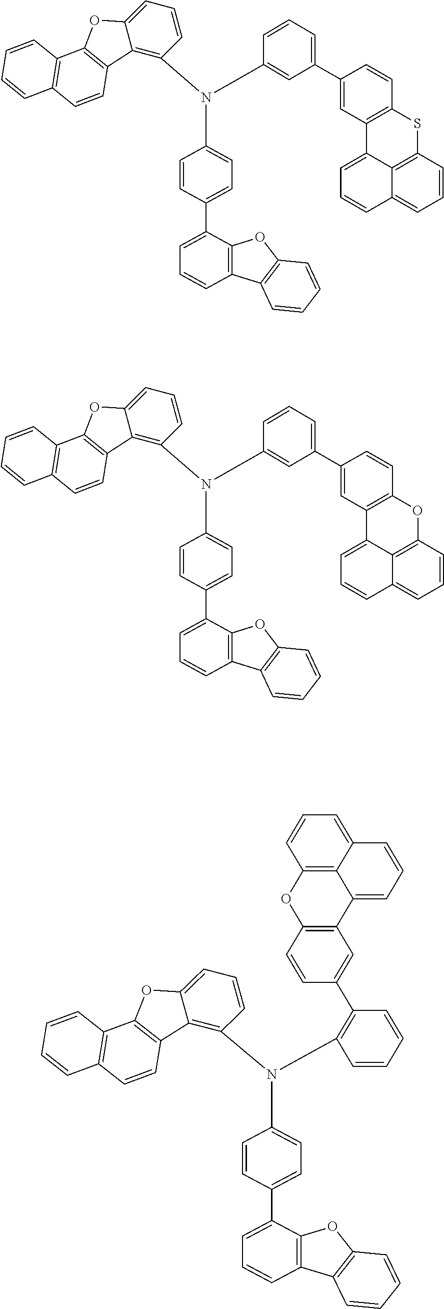 [SMILES: c1cc(-c2ccc3c(c2)-c2cccc4cccc(c24)S3)cc(N(c2ccc(-c3cccc4c3oc3ccccc34)cc2)c2cccc3oc4c5ccccc5ccc4c23)c1 ; c1cc(-c2ccc3c(c2)-c2cccc4cccc(c24)O3)cc(N(c2ccc(-c3cccc4c3oc3ccccc34)cc2)c2cccc3oc4c5ccccc5ccc4c23)c1 ; c1ccc(N(c2ccc(-c3cccc4c3oc3ccccc34)cc2)c2cccc3oc4c5ccccc5ccc4c23)c(-c2ccc3c(c2)-c2cccc4cccc(c24)O3)c1]

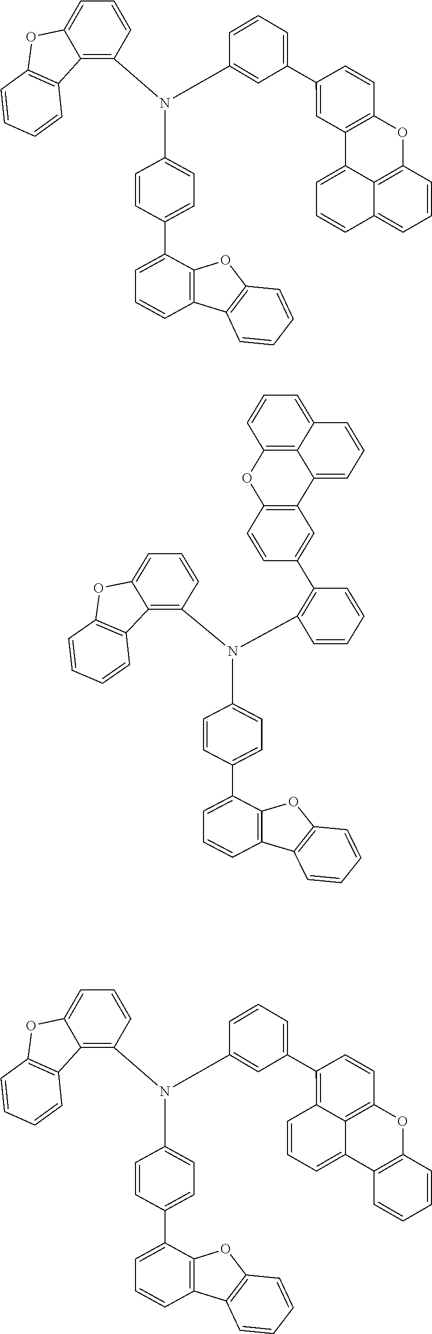 [SMILES: c1cc(-c2ccc3c(c2)-c2cccc4cccc(c24)O3)cc(N(c2ccc(-c3cccc4c3oc3ccccc34)cc2)c2cccc3oc4ccccc4c23)c1 ; c1ccc(N(c2ccc(-c3cccc4c3oc3ccccc34)cc2)c2cccc3oc4ccccc4c23)c(-c2ccc3c(c2)-c2cccc4cccc(c24)O3)c1 ; c1cc(-c2ccc3c4c(cccc24)-c2ccccc2O3)cc(N(c2ccc(-c3cccc4c3oc3ccccc34)cc2)c2cccc3oc4ccccc4c23)c1]

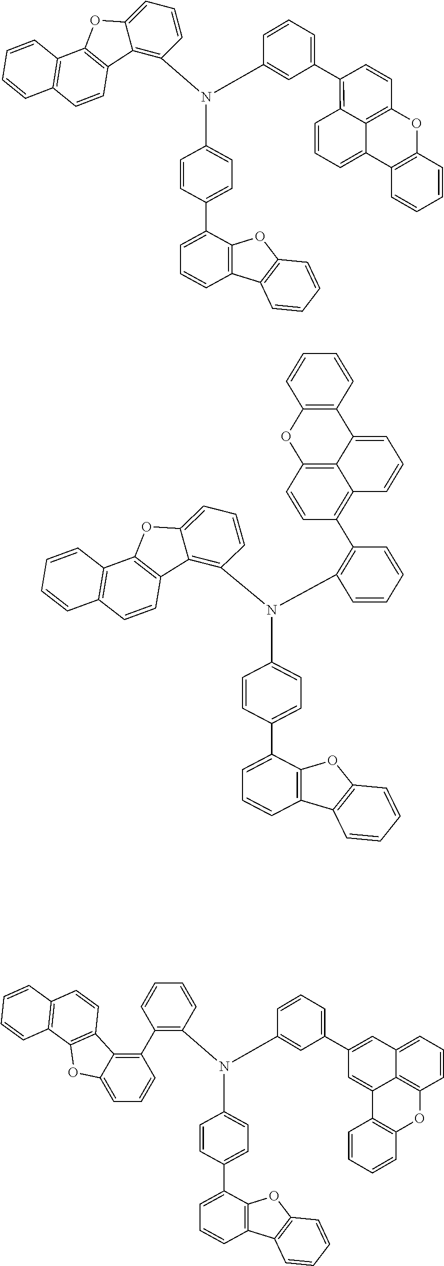 [SMILES: c1cc(-c2ccc3c4c(cccc24)-c2ccccc2O3)cc(N(c2ccc(-c3cccc4c3oc3ccccc34)cc2)c2cccc3oc4c5ccccc5ccc4c23)c1 ; c1ccc2c(c1)Oc1ccc(-c3ccccc3N(c3ccc(-c4cccc5c4oc4ccccc45)cc3)c3cccc4oc5c6ccccc6ccc5c34)c3cccc-2c13 ; c1cc(-c2cc3c4c(cccc4c2)Oc2ccccc2-3)cc(N(c2ccc(-c3cccc4c3oc3ccccc34)cc2)c2ccccc2-c2cccc3oc4c5ccccc5ccc4c23)c1]

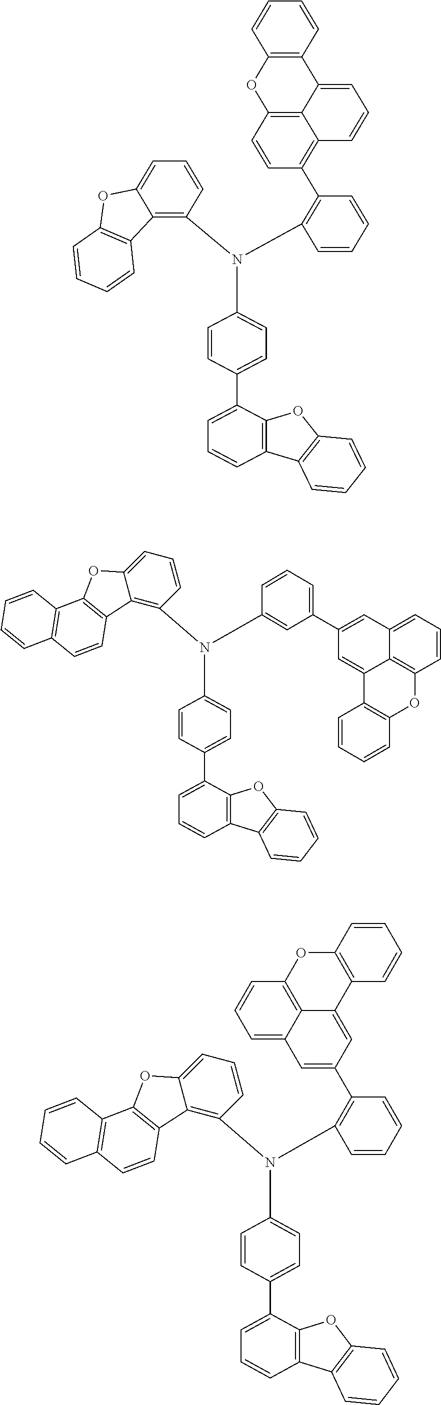 [SMILES: c1ccc2c(c1)Oc1ccc(-c3ccccc3N(c3ccc(-c4cccc5c4oc4ccccc45)cc3)c3cccc4oc5ccccc5c34)c3cccc-2c13 ; c1cc(-c2cc3c4c(cccc4c2)Oc2ccccc2-3)cc(N(c2ccc(-c3cccc4c3oc3ccccc34)cc2)c2cccc3oc4c5ccccc5ccc4c23)c1 ; c1ccc2c(c1)Oc1cccc3cc(-c4ccccc4N(c4ccc(-c5cccc6c5oc5ccccc56)cc4)c4cccc5oc6c7ccccc7ccc6c45)cc-2c13]

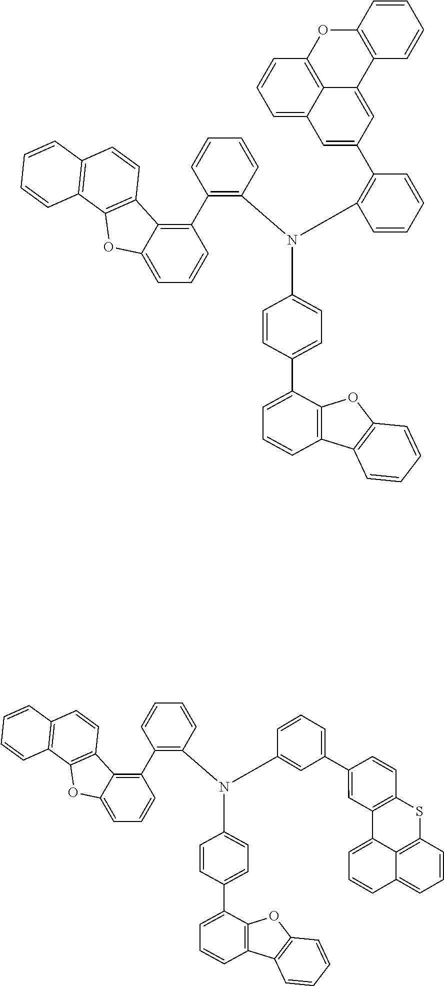 [SMILES: c1ccc2c(c1)Oc1cccc3cc(-c4ccccc4N(c4ccc(-c5cccc6c5oc5ccccc56)cc4)c4ccccc4-c4cccc5oc6c7ccccc7ccc6c45)cc-2c13 ; c1cc(-c2ccc3c(c2)-c2cccc4cccc(c24)S3)cc(N(c2ccc(-c3cccc4c3oc3ccccc34)cc2)c2ccccc2-c2cccc3oc4c5ccccc5ccc4c23)c1]

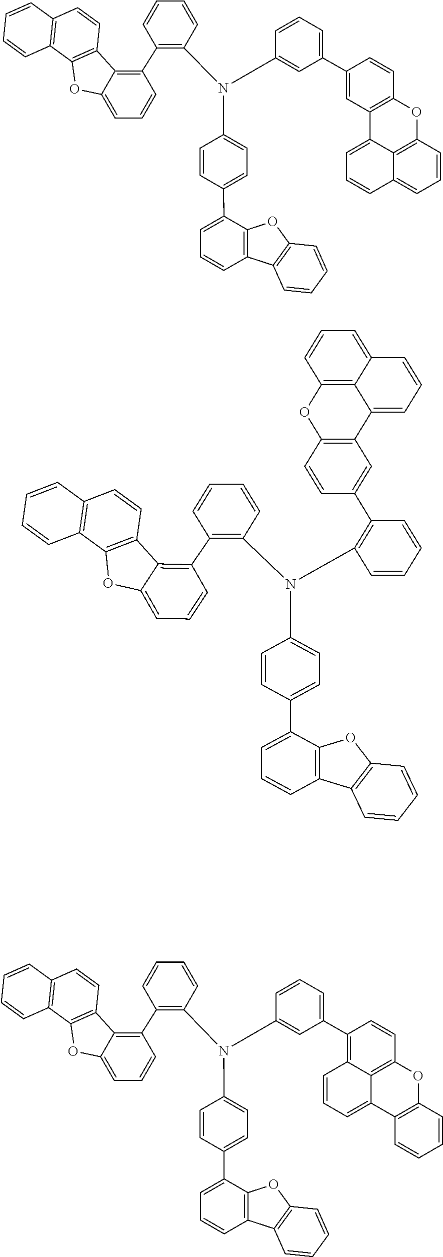 [SMILES: c1cc(-c2ccc3c(c2)-c2cccc4cccc(c24)O3)cc(N(c2ccc(-c3cccc4c3oc3ccccc34)cc2)c2ccccc2-c2cccc3oc4c5ccccc5ccc4c23)c1 ; c1ccc(N(c2ccc(-c3cccc4c3oc3ccccc34)cc2)c2ccccc2-c2cccc3oc4c5ccccc5ccc4c23)c(-c2ccc3c(c2)-c2cccc4cccc(c24)O3)c1 ; c1cc(-c2ccc3c4c(cccc24)-c2ccccc2O3)cc(N(c2ccc(-c3cccc4c3oc3ccccc34)cc2)c2ccccc2-c2cccc3oc4c5ccccc5ccc4c23)c1]

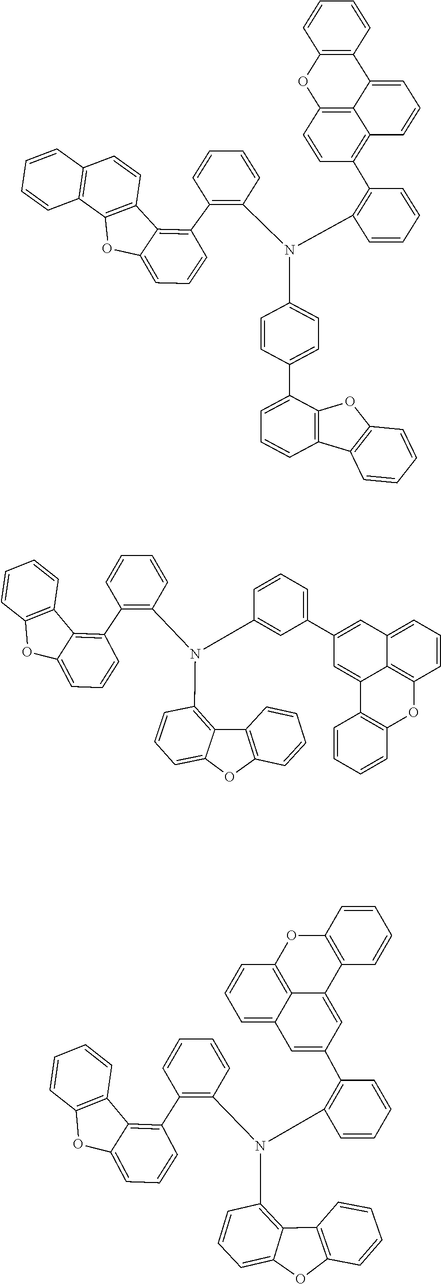 [SMILES: c1ccc2c(c1)Oc1ccc(-c3ccccc3N(c3ccc(-c4cccc5c4oc4ccccc45)cc3)c3ccccc3-c3cccc4oc5c6ccccc6ccc5c34)c3cccc-2c13 ; c1cc(-c2cc3c4c(cccc4c2)Oc2ccccc2-3)cc(N(c2ccccc2-c2cccc3oc4ccccc4c23)c2cccc3oc4ccccc4c23)c1 ; c1ccc2c(c1)Oc1cccc3cc(-c4ccccc4N(c4ccccc4-c4cccc5oc6ccccc6c45)c4cccc5oc6ccccc6c45)cc-2c13]

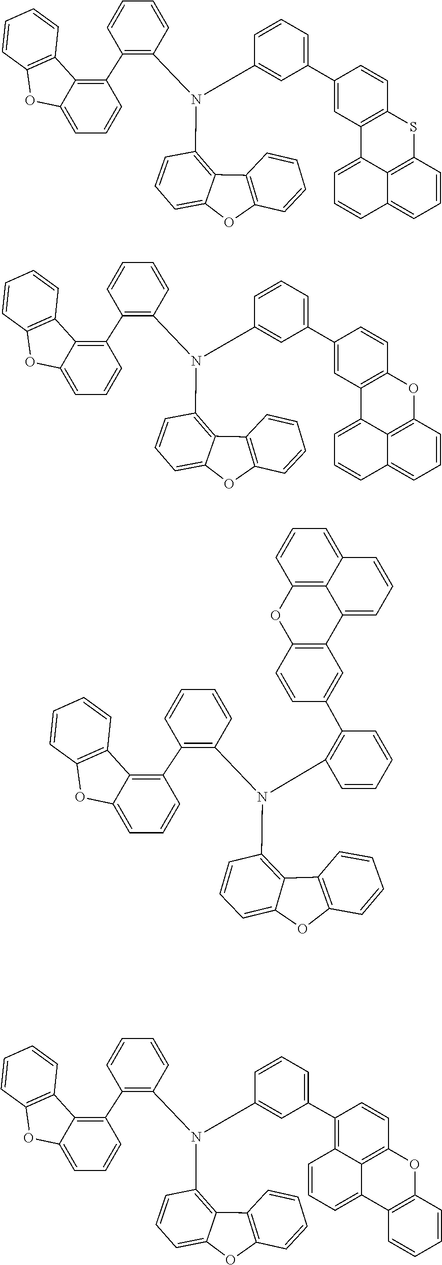 [SMILES: c1cc(-c2ccc3c(c2)-c2cccc4cccc(c24)S3)cc(N(c2ccccc2-c2cccc3oc4ccccc4c23)c2cccc3oc4ccccc4c23)c1 ; c1cc(-c2ccc3c(c2)-c2cccc4cccc(c24)O3)cc(N(c2ccccc2-c2cccc3oc4ccccc4c23)c2cccc3oc4ccccc4c23)c1 ; c1ccc(N(c2ccccc2-c2cccc3oc4ccccc4c23)c2cccc3oc4ccccc4c23)c(-c2ccc3c(c2)-c2cccc4cccc(c24)O3)c1 ; c1cc(-c2ccc3c4c(cccc24)-c2ccccc2O3)cc(N(c2ccccc2-c2cccc3oc4ccccc4c23)c2cccc3oc4ccccc4c23)c1]

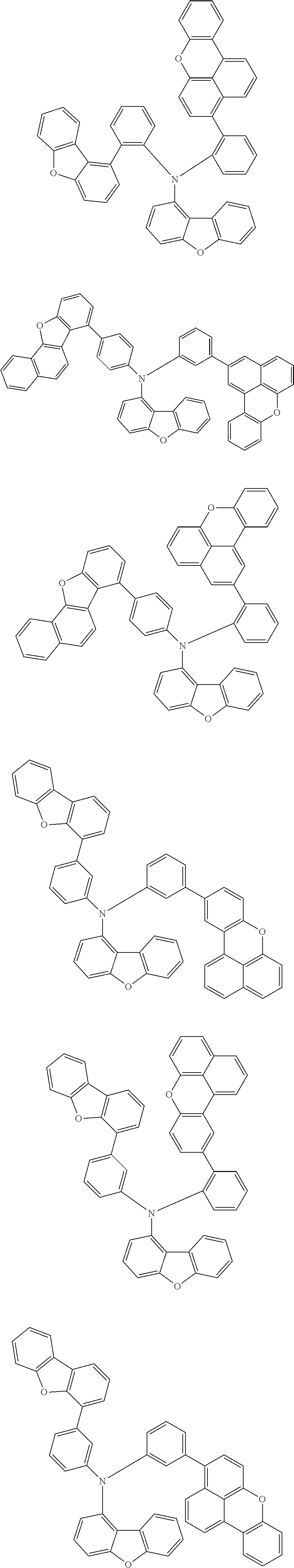

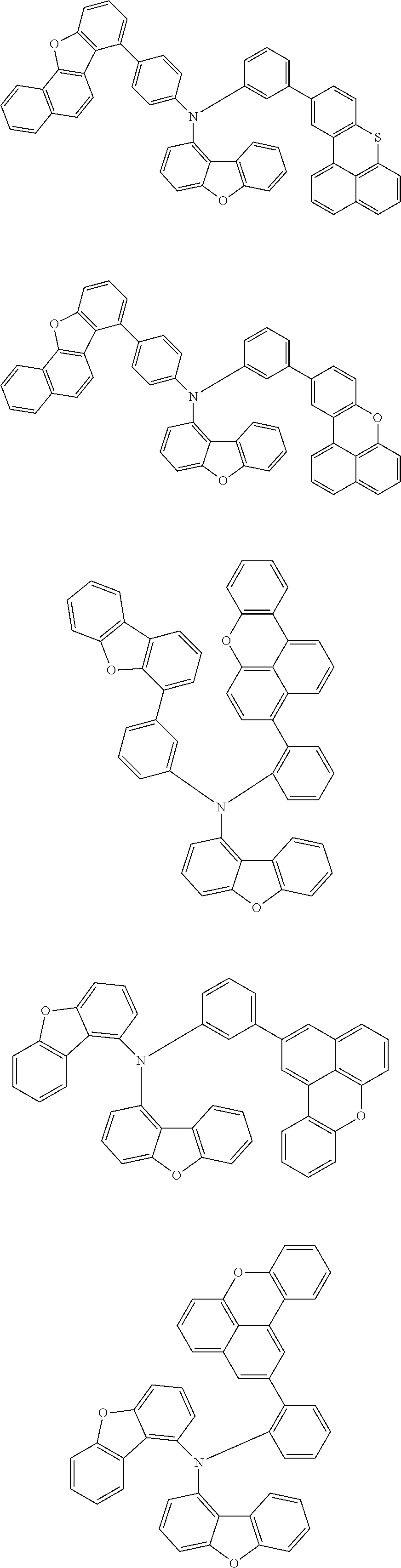 [SMILES: c1cc(-c2ccc3c(c2)-c2cccc4cccc(c24)S3)cc(N(c2ccc(-c3cccc4oc5c6ccccc6ccc5c34)cc2)c2cccc3oc4ccccc4c23)c1 ; c1cc(-c2ccc3c(c2)-c2cccc4cccc(c24)O3)cc(N(c2ccc(-c3cccc4oc5c6ccccc6ccc5c34)cc2)c2cccc3oc4ccccc4c23)c1 ; c1cc(-c2cccc3c2oc2ccccc23)cc(N(c2ccccc2-c2ccc3c4c(cccc24)-c2ccccc2O3)c2cccc3oc4ccccc4c23)c1 ; c1cc(-c2cc3c4c(cccc4c2)Oc2ccccc2-3)cc(N(c2cccc3oc4ccccc4c23)c2cccc3oc4ccccc4c23)c1 ; c1ccc2c(c1)Oc1cccc3cc(-c4ccccc4N(c4cccc5oc6ccccc6c45)c4cccc5oc6ccccc6c45)cc-2c13]

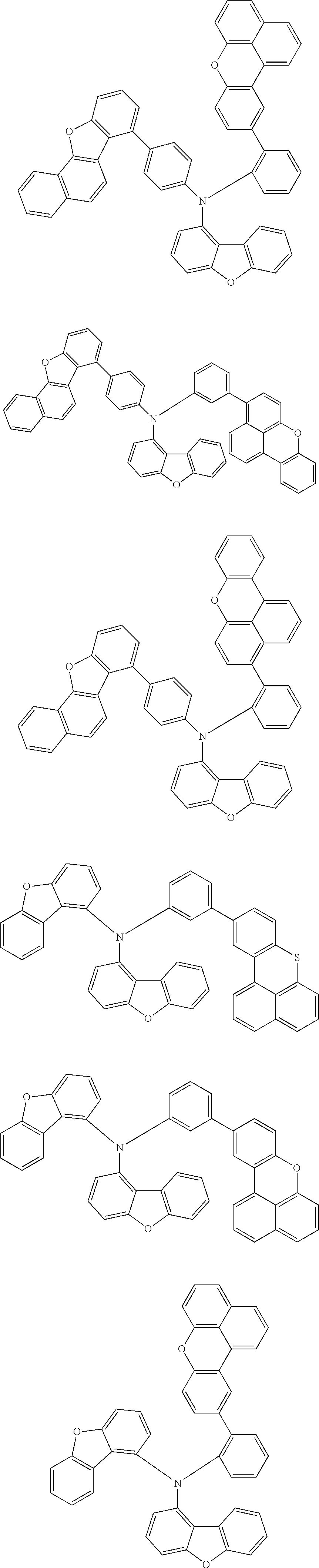

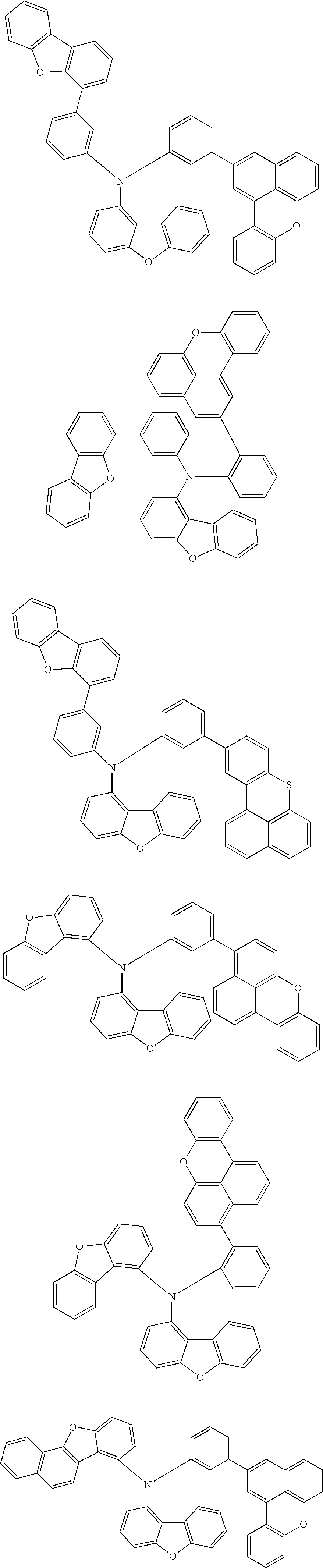

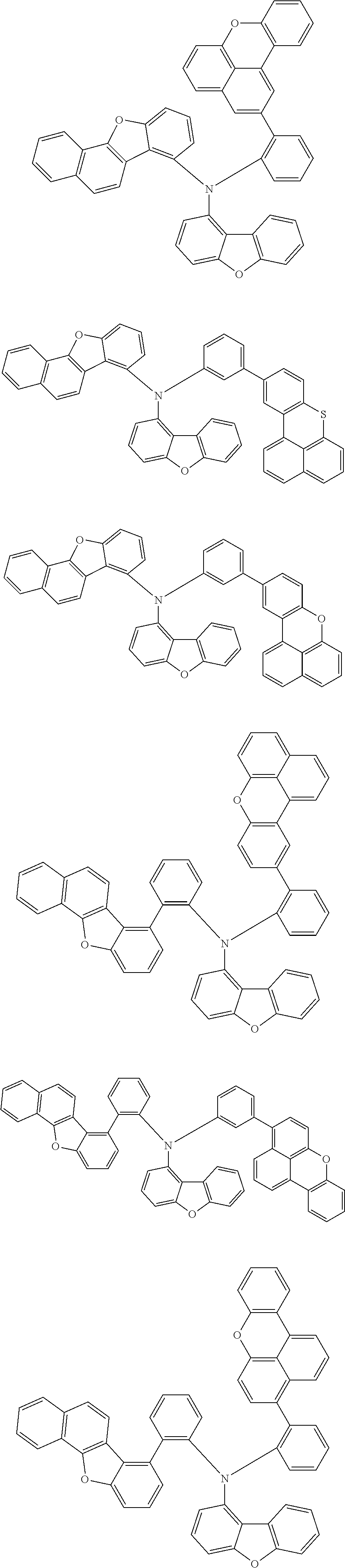

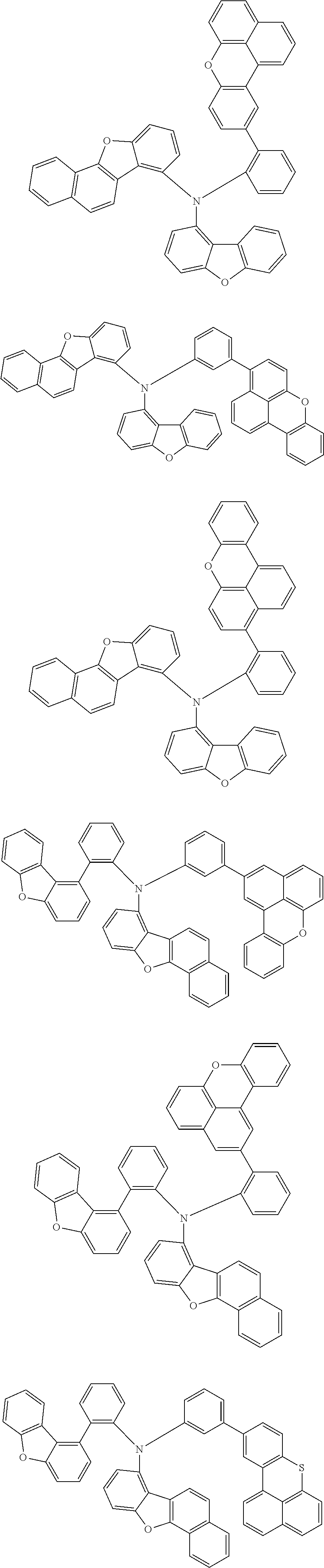

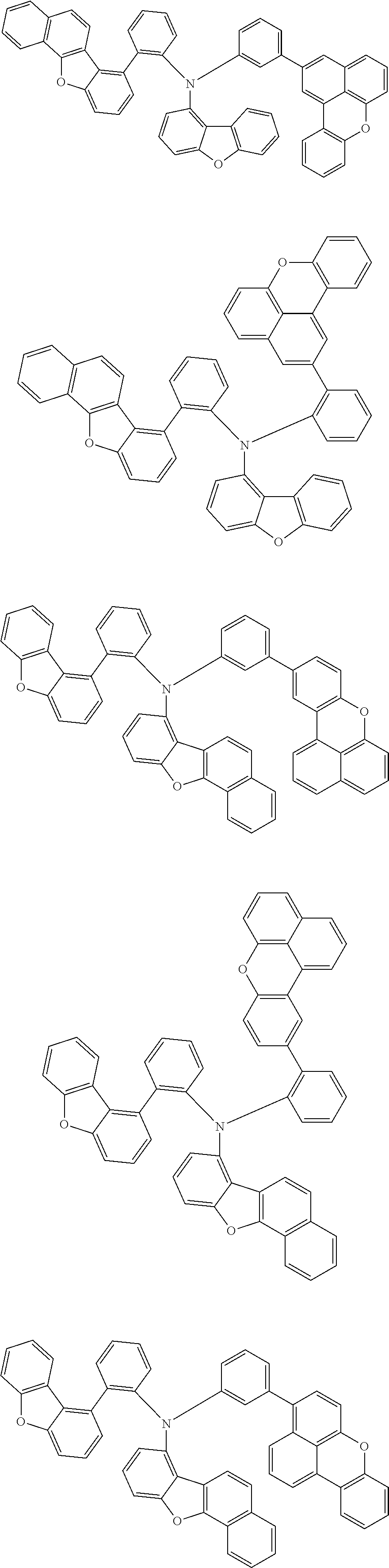 [SMILES: c1cc(-c2cc3c4c(cccc4c2)Oc2ccccc2-3)cc(N(c2ccccc2-c2cccc3oc4c5ccccc5ccc4c23)c2cccc3oc4ccccc4c23)c1 ; c1ccc2c(c1)Oc1cccc3cc(-c4ccccc4N(c4ccccc4-c4cccc5oc6c7ccccc7ccc6c45)c4cccc5oc6ccccc6c45)cc-2c13 ; c1cc(-c2ccc3c(c2)-c2cccc4cccc(c24)O3)cc(N(c2ccccc2-c2cccc3oc4ccccc4c23)c2cccc3oc4c5ccccc5ccc4c23)c1 ; c1ccc(N(c2ccccc2-c2cccc3oc4ccccc4c23)c2cccc3oc4c5ccccc5ccc4c23)c(-c2ccc3c(c2)-c2cccc4cccc(c24)O3)c1 ; c1cc(-c2ccc3c4c(cccc24)-c2ccccc2O3)cc(N(c2ccccc2-c2cccc3oc4ccccc4c23)c2cccc3oc4c5ccccc5ccc4c23)c1]

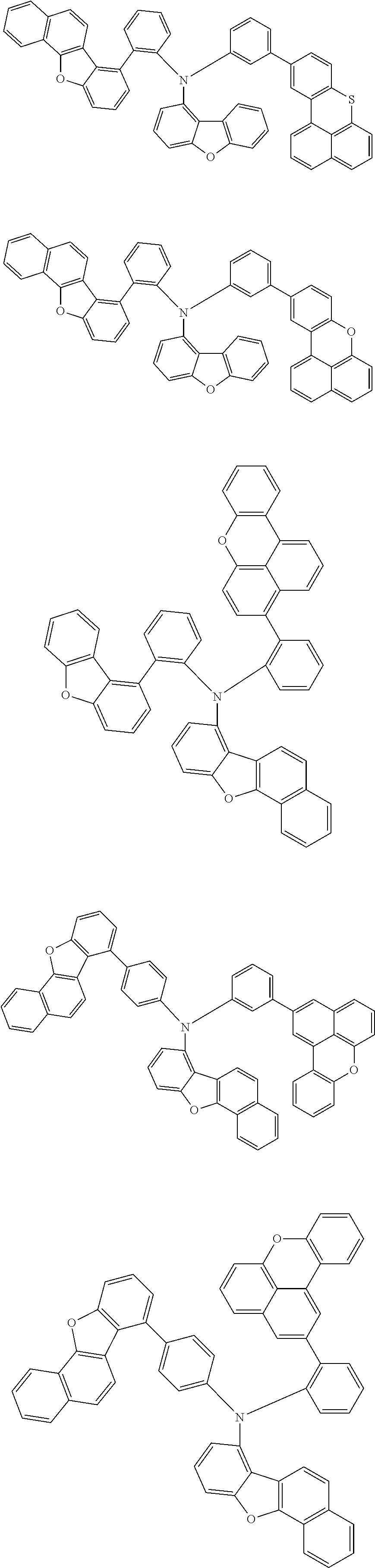

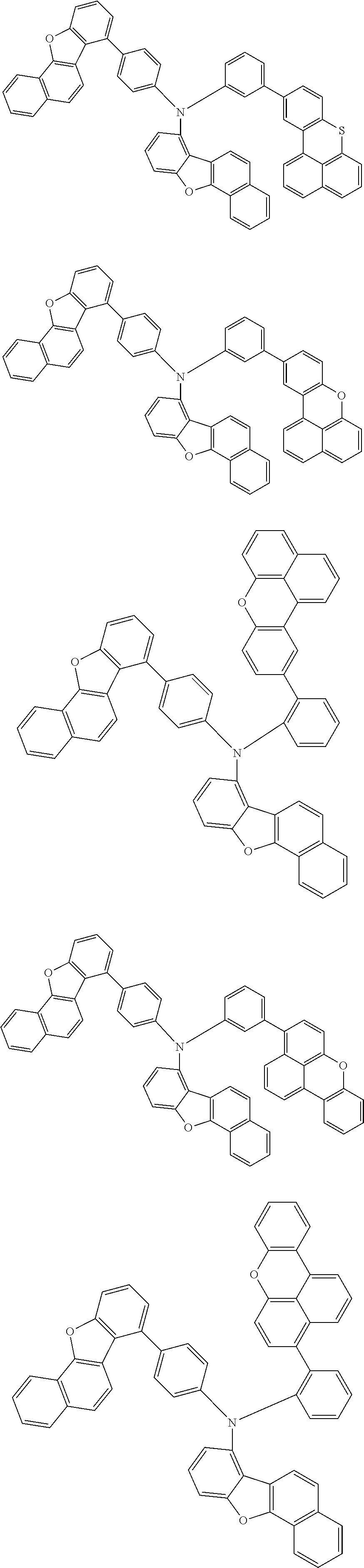

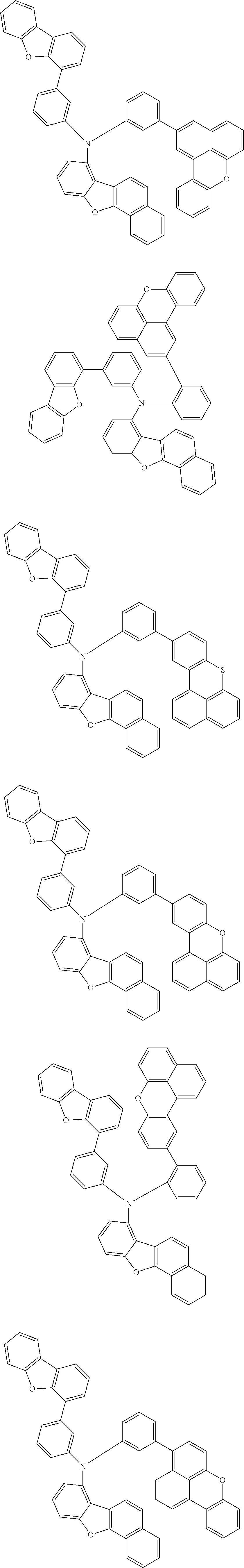

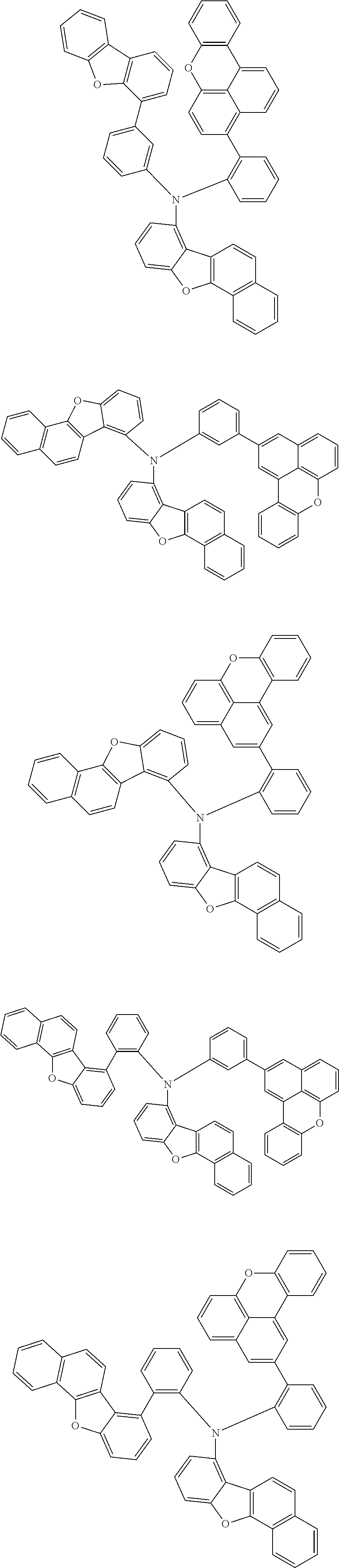 [SMILES: c1cc(-c2cccc3c2oc2ccccc23)cc(N(c2ccccc2-c2ccc3c4c(cccc24)-c2ccccc2O3)c2cccc3oc4c5ccccc5ccc4c23)c1 ; c1cc(-c2cc3c4c(cccc4c2)Oc2ccccc2-3)cc(N(c2cccc3oc4c5ccccc5ccc4c23)c2cccc3oc4c5ccccc5ccc4c23)c1 ; c1ccc2c(c1)Oc1cccc3cc(-c4ccccc4N(c4cccc5oc6c7ccccc7ccc6c45)c4cccc5oc6c7ccccc7ccc6c45)cc-2c13 ; c1cc(-c2cc3c4c(cccc4c2)Oc2ccccc2-3)cc(N(c2ccccc2-c2cccc3oc4c5ccccc5ccc4c23)c2cccc3oc4c5ccccc5ccc4c23)c1 ; c1ccc2c(c1)Oc1cccc3cc(-c4ccccc4N(c4ccccc4-c4cccc5oc6c7ccccc7ccc6c45)c4cccc5oc6c7ccccc7ccc6c45)cc-2c13]

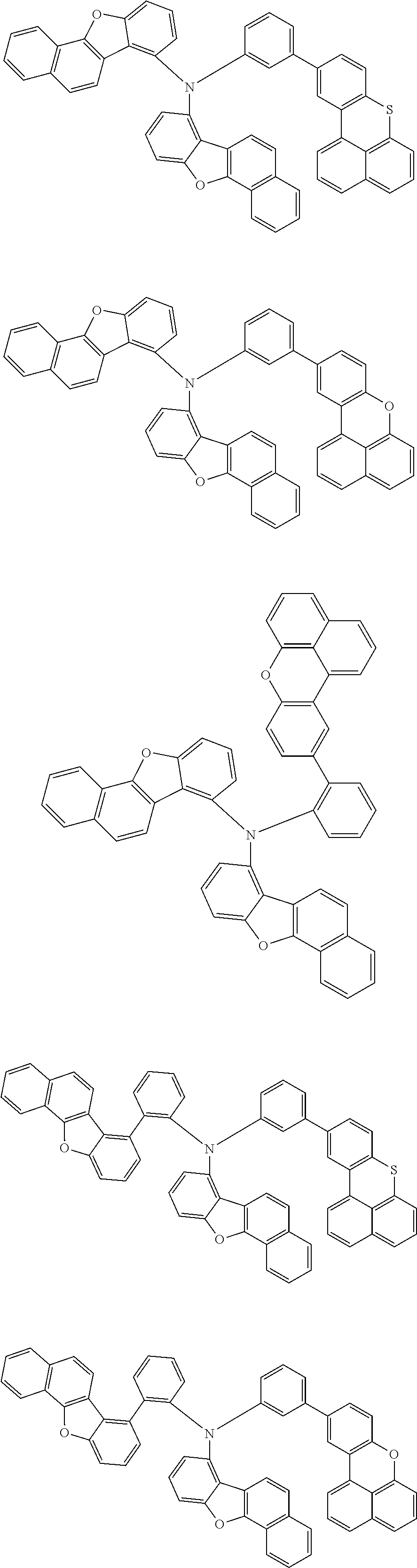

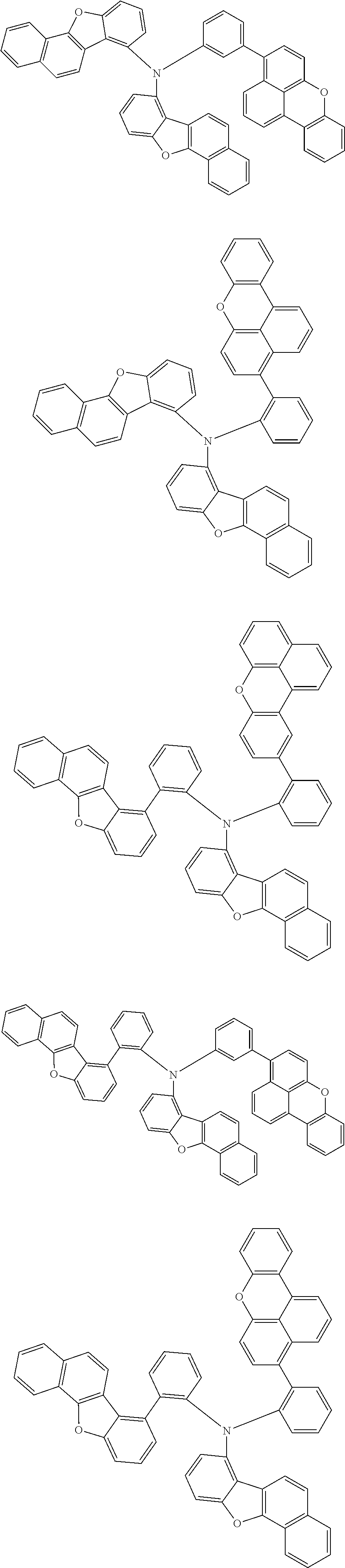

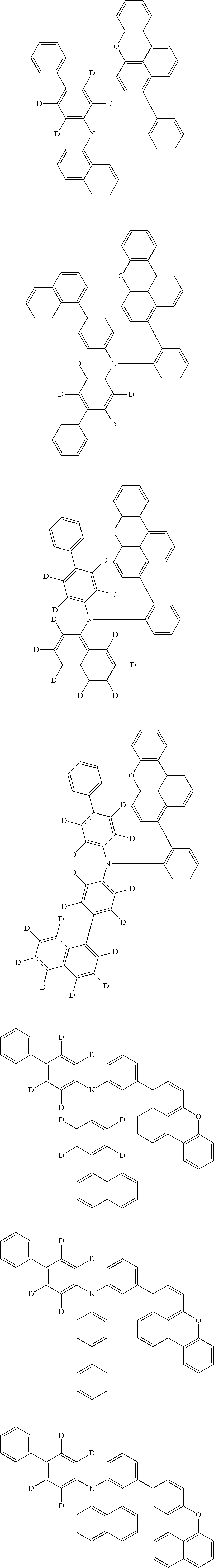

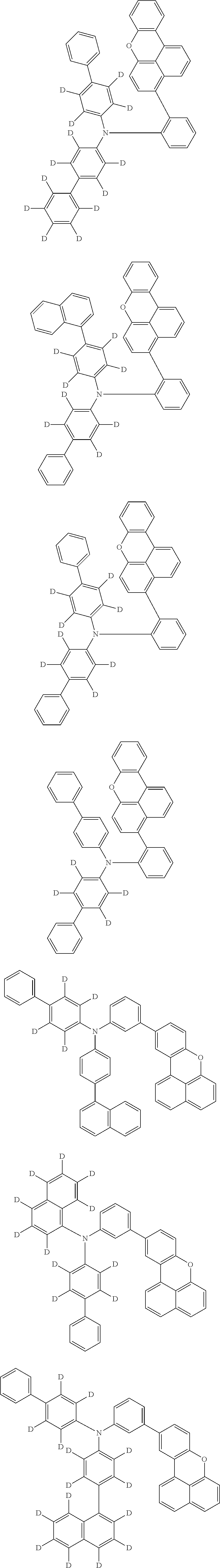

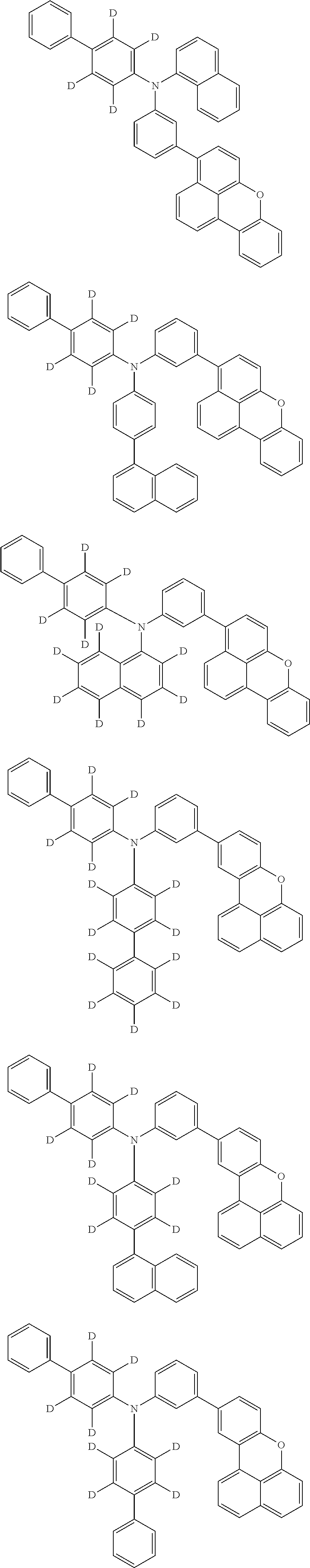

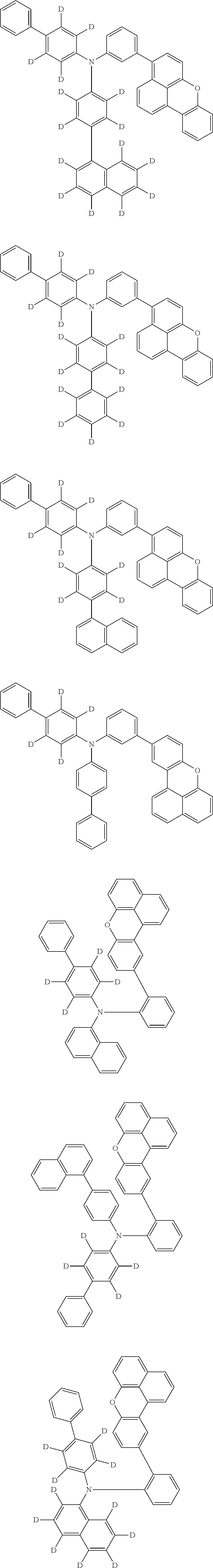

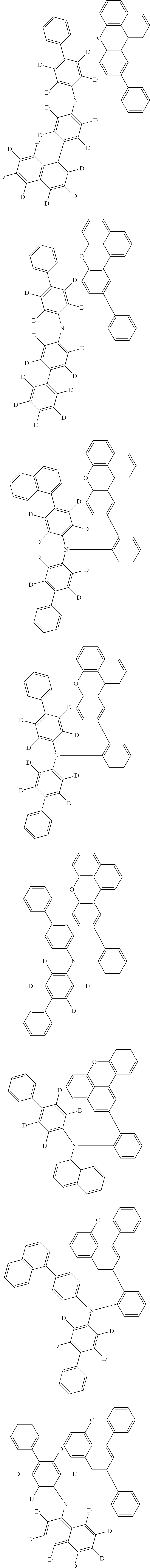

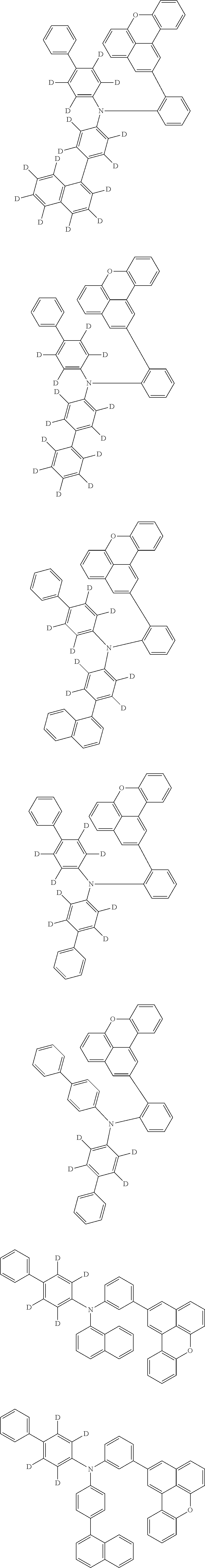

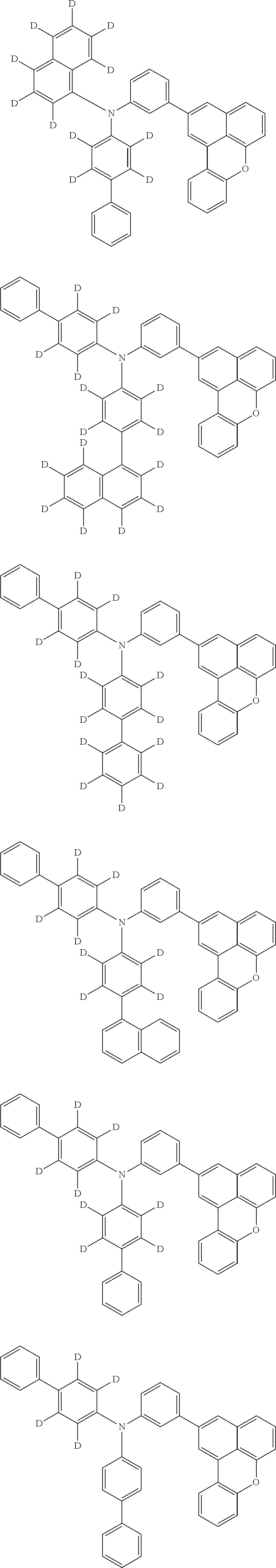

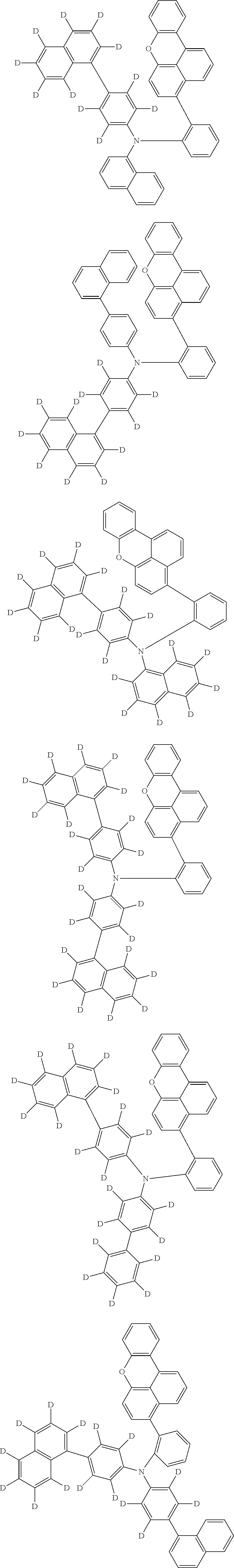

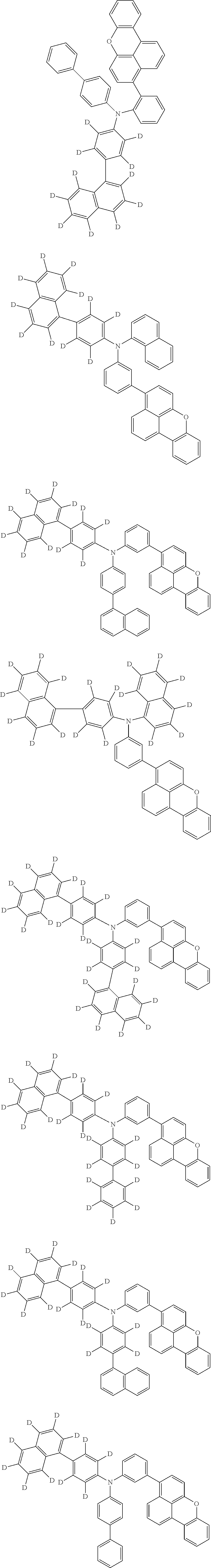

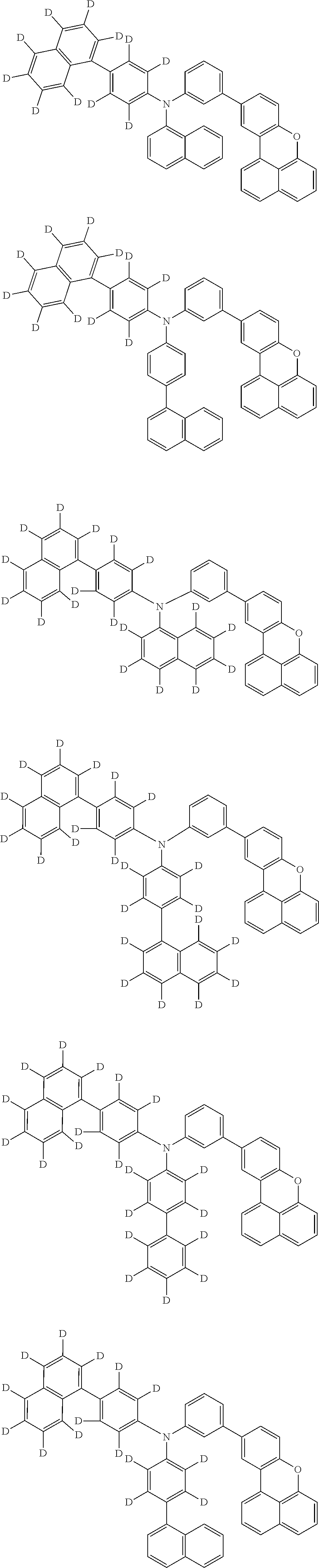

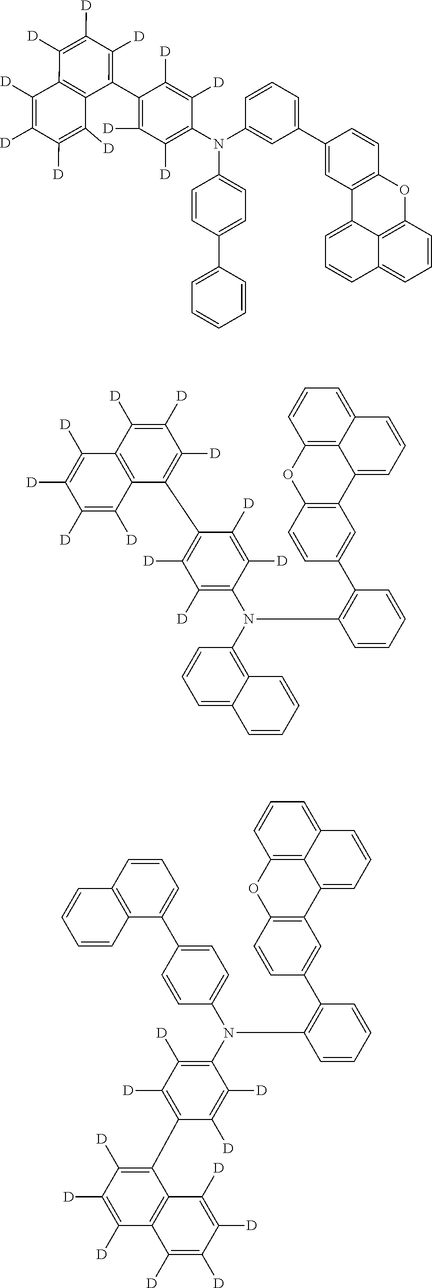 [SMILES: [2H]c1c([2H])c(N(c2ccc(-c3ccccc3)cc2)c2cccc(-c3ccc4c(c3)-c3cccc5cccc(c35)O4)c2)c([2H])c([2H])c1-c1c([2H])c([2H])c([2H])c2c([2H])c([2H])c([2H])c([2H])c12 ; [2H]c1c([2H])c(N(c2ccccc2-c2ccc3c(c2)-c2cccc4cccc(c24)O3)c2cccc3ccccc23)c([2H])c([2H])c1-c1c([2H])c([2H])c([2H])c2c([2H])c([2H])c([2H])c([2H])c12 ; [2H]c1c([2H])c(N(c2ccc(-c3cccc4ccccc34)cc2)c2ccccc2-c2ccc3c(c2)-c2cccc4cccc(c24)O3)c([2H])c([2H])c1-c1c([2H])c([2H])c([2H])c2c([2H])c([2H])c([2H])c([2H])c12]

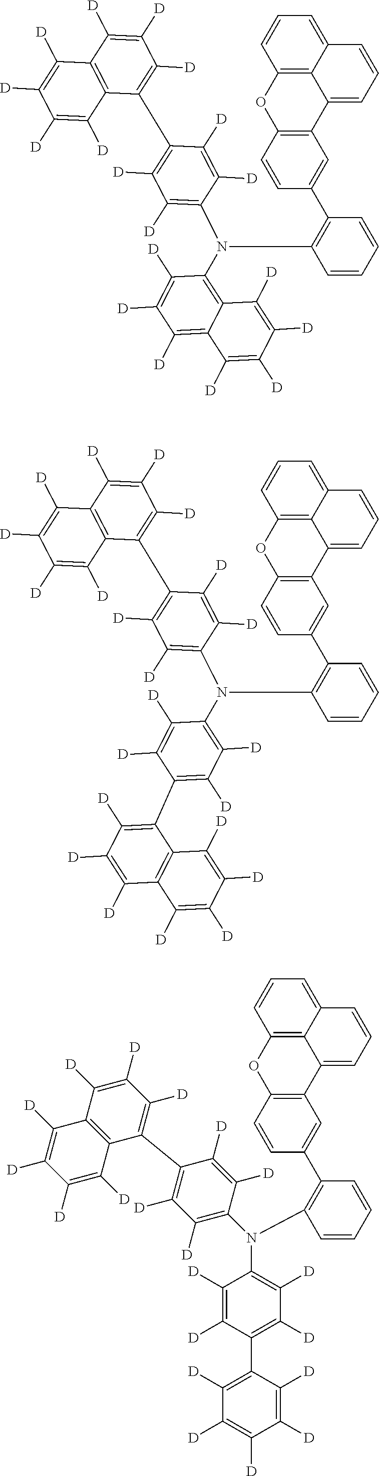

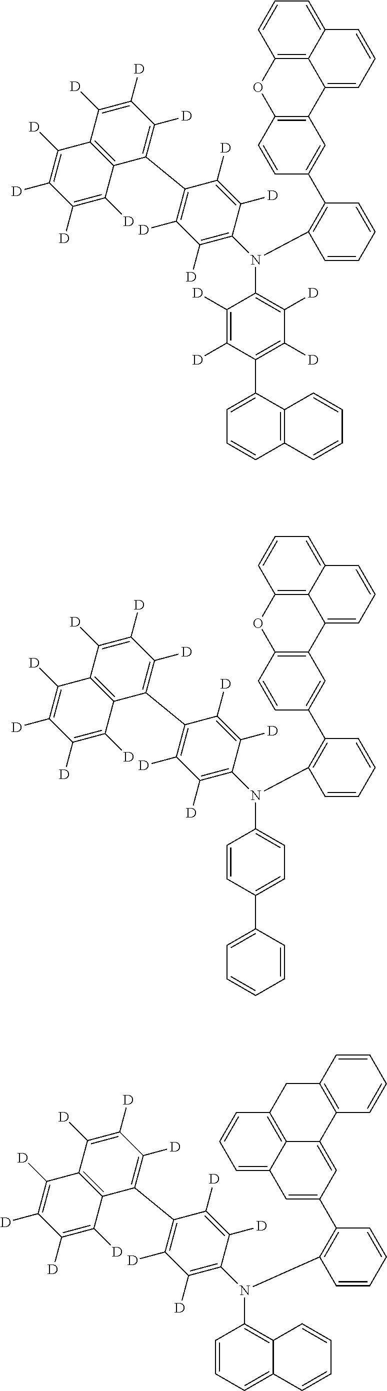 [SMILES: [2H]c1c([2H])c(N(c2ccccc2-c2ccc3c(c2)-c2cccc4cccc(c24)O3)c2c([2H])c([2H])c(-c3c([2H])c([2H])c([2H])c4c([2H])c([2H])c([2H])c([2H])c34)c([2H])c2[2H])c([2H])c([2H])c1-c1cccc2ccccc12 ; [2H]c1c([2H])c(N(c2ccc(-c3ccccc3)cc2)c2ccccc2-c2ccc3c(c2)-c2cccc4cccc(c24)O3)c([2H])c([2H])c1-c1c([2H])c([2H])c([2H])c2c([2H])c([2H])c([2H])c([2H])c12 ; [2H]c1c([2H])c(N(c2ccccc2-c2cc3c4c(cccc4c2)Cc2ccccc2-3)c2cccc3ccccc23)c([2H])c([2H])c1-c1c([2H])c([2H])c([2H])c2c([2H])c([2H])c([2H])c([2H])c12]

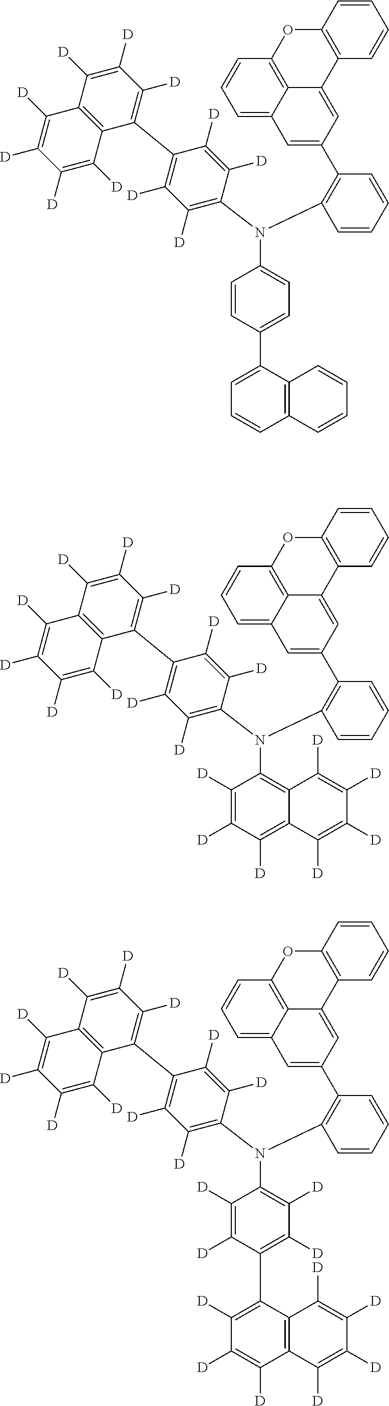

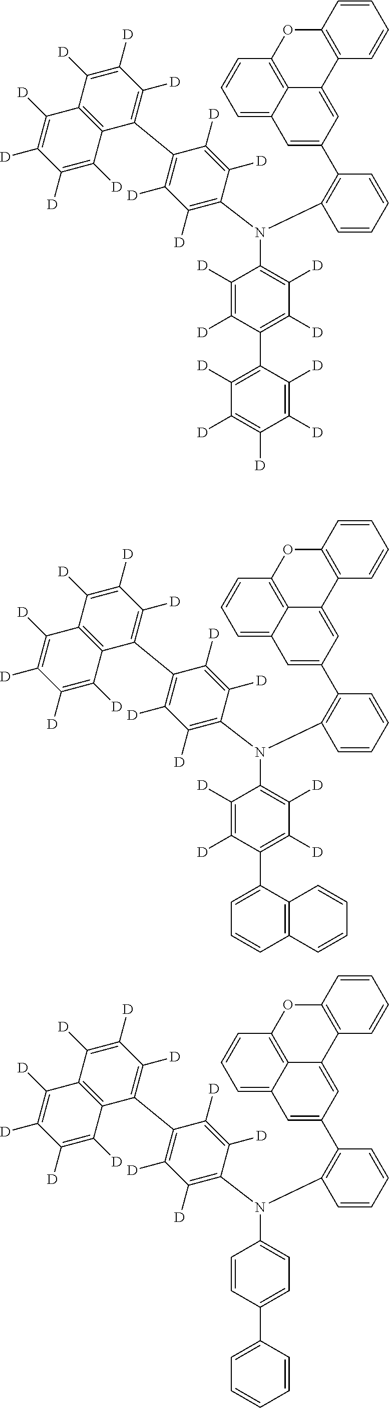

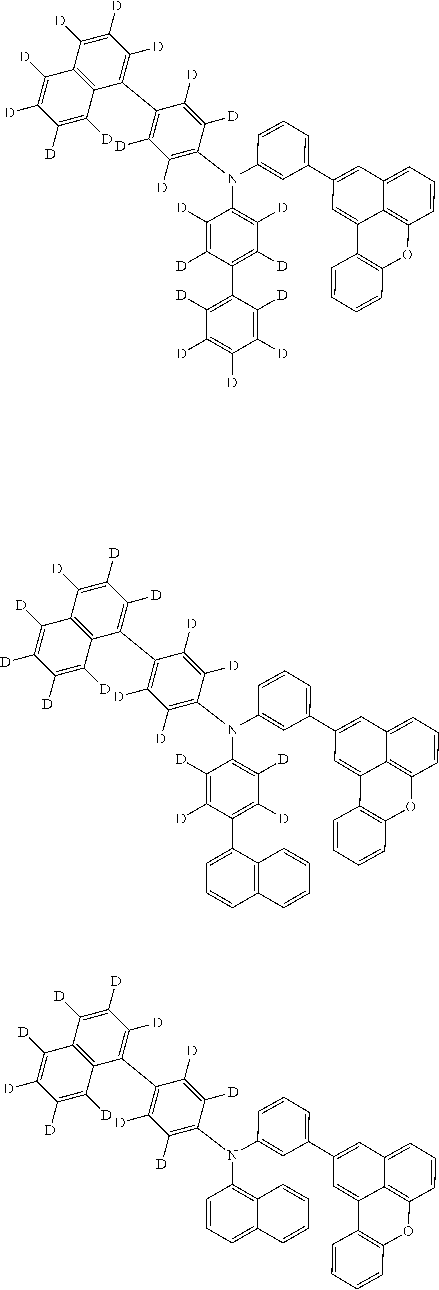

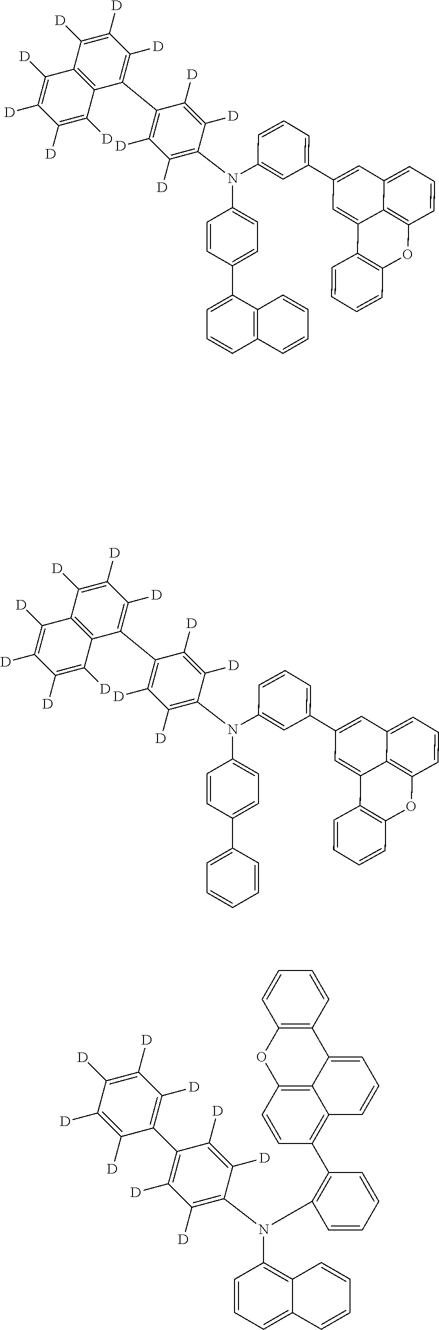 [SMILES: [2H]c1c([2H])c(N(c2ccc(-c3cccc4ccccc34)cc2)c2cccc(-c3cc4c5c(cccc5c3)Oc3ccccc3-4)c2)c([2H])c([2H])c1-c1c([2H])c([2H])c([2H])c2c([2H])c([2H])c([2H])c([2H])c12 ; [2H]c1c([2H])c(N(c2ccc(-c3ccccc3)cc2)c2cccc(-c3cc4c5c(cccc5c3)Oc3ccccc3-4)c2)c([2H])c([2H])c1-c1c([2H])c([2H])c([2H])c2c([2H])c([2H])c([2H])c([2H])c12 ; [2H]c1c([2H])c([2H])c(-c2c([2H])c([2H])c(N(c3ccccc3-c3ccc4c5c(cccc35)-c3ccccc3O4)c3cccc4ccccc34)c([2H])c2[2H])c([2H])c1[2H]]

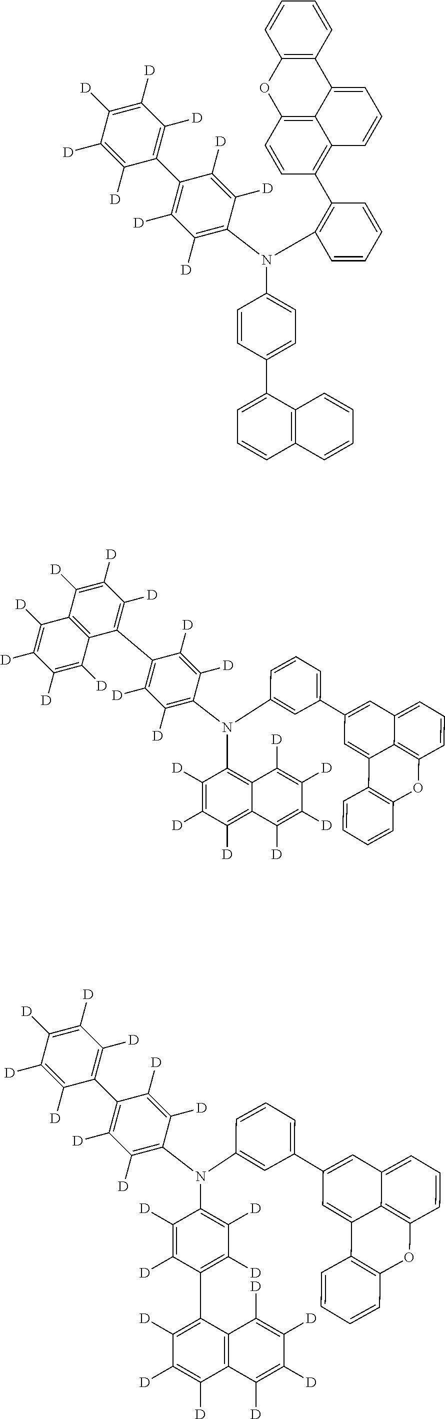

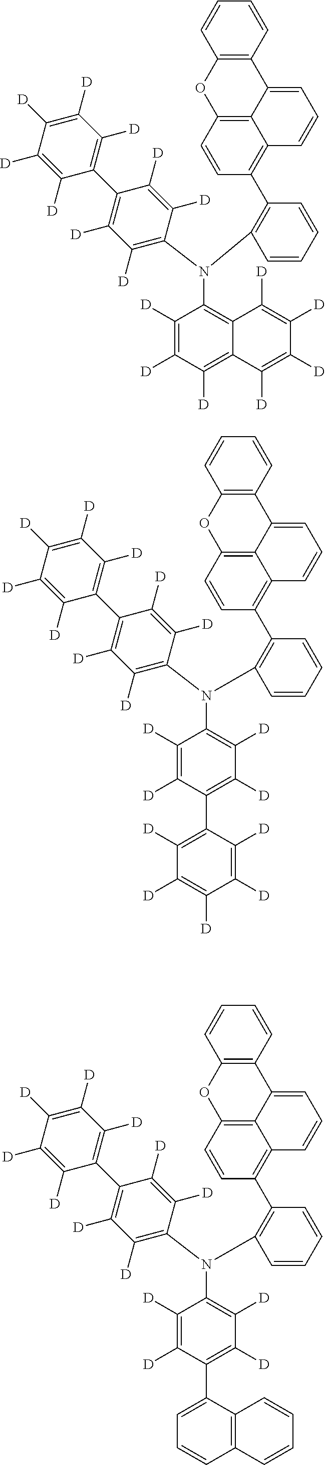

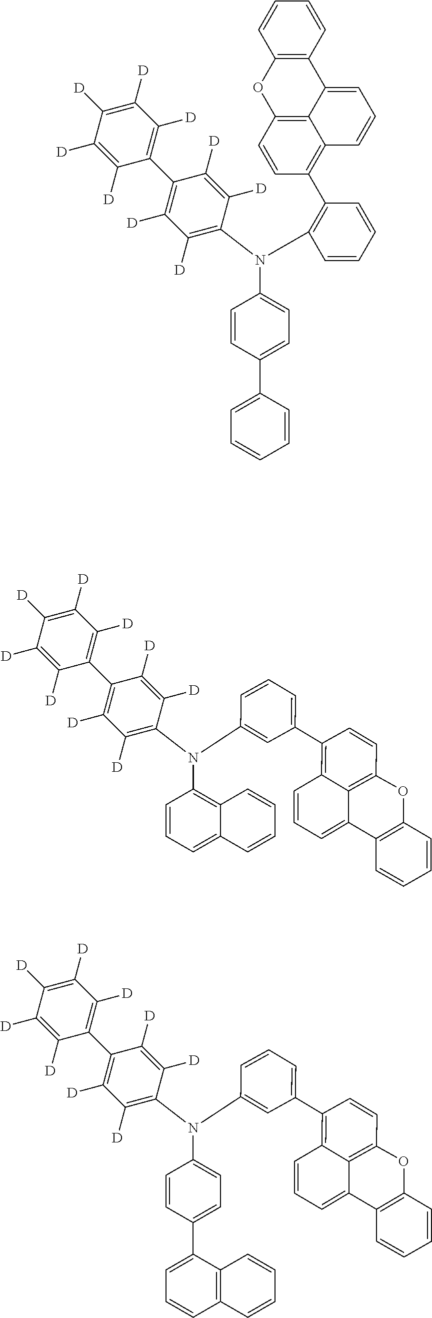 [SMILES: [2H]c1c([2H])c([2H])c(-c2c([2H])c([2H])c(N(c3ccc(-c4ccccc4)cc3)c3ccccc3-c3ccc4c5c(cccc35)-c3ccccc3O4)c([2H])c2[2H])c([2H])c1[2H] ; [2H]c1c([2H])c([2H])c(-c2c([2H])c([2H])c(N(c3cccc(-c4ccc5c6c(cccc46)-c4ccccc4O5)c3)c3cccc4ccccc34)c([2H])c2[2H])c([2H])c1[2H] ; [2H]c1c([2H])c([2H])c(-c2c([2H])c([2H])c(N(c3ccc(-c4cccc5ccccc45)cc3)c3cccc(-c4ccc5c6c(cccc46)-c4ccccc4O5)c3)c([2H])c2[2H])c([2H])c1[2H]]

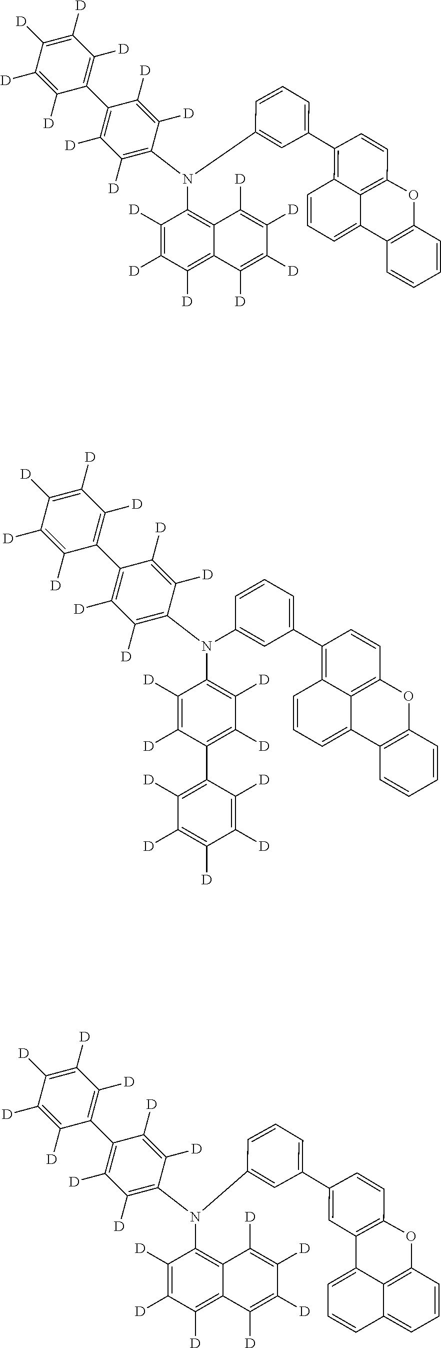

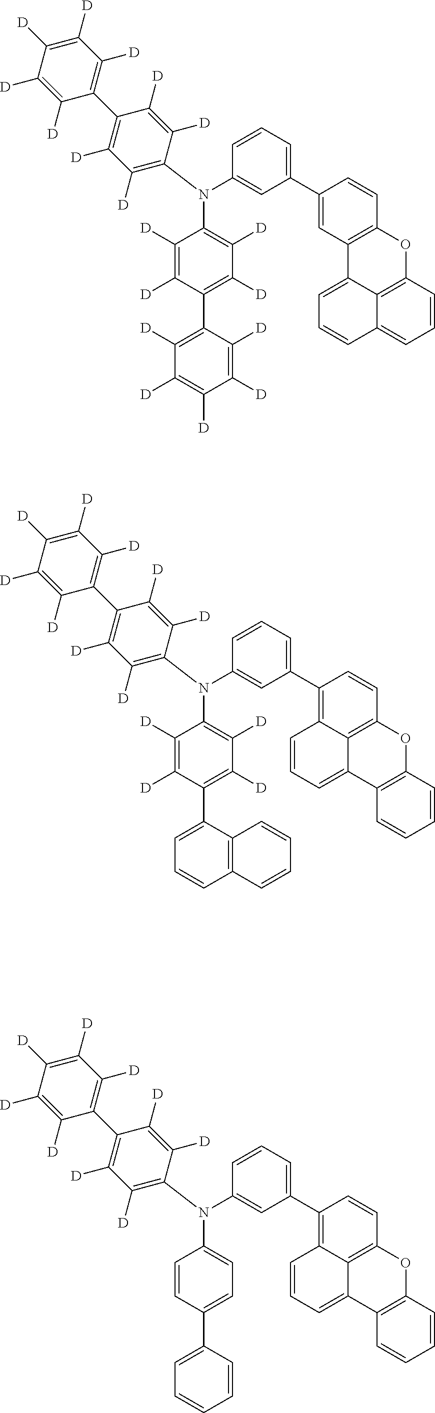 [SMILES: [2H]c1c([2H])c([2H])c(-c2c([2H])c([2H])c(N(c3cccc(-c4ccc5c(c4)-c4cccc6cccc(c46)O5)c3)c3c([2H])c([2H])c(-c4c([2H])c([2H])c([2H])c([2H])c4[2H])c([2H])c3[2H])c([2H])c2[2H])c([2H])c1[2H] ; [2H]c1c([2H])c([2H])c(-c2c([2H])c([2H])c(N(c3cccc(-c4ccc5c6c(cccc46)-c4ccccc4O5)c3)c3c([2H])c([2H])c(-c4cccc5ccccc45)c([2H])c3[2H])c([2H])c2[2H])c([2H])c1[2H] ; [2H]c1c([2H])c([2H])c(-c2c([2H])c([2H])c(N(c3ccc(-c4ccccc4)cc3)c3cccc(-c4ccc5c6c(cccc46)-c4ccccc4O5)c3)c([2H])c2[2H])c([2H])c1[2H]]

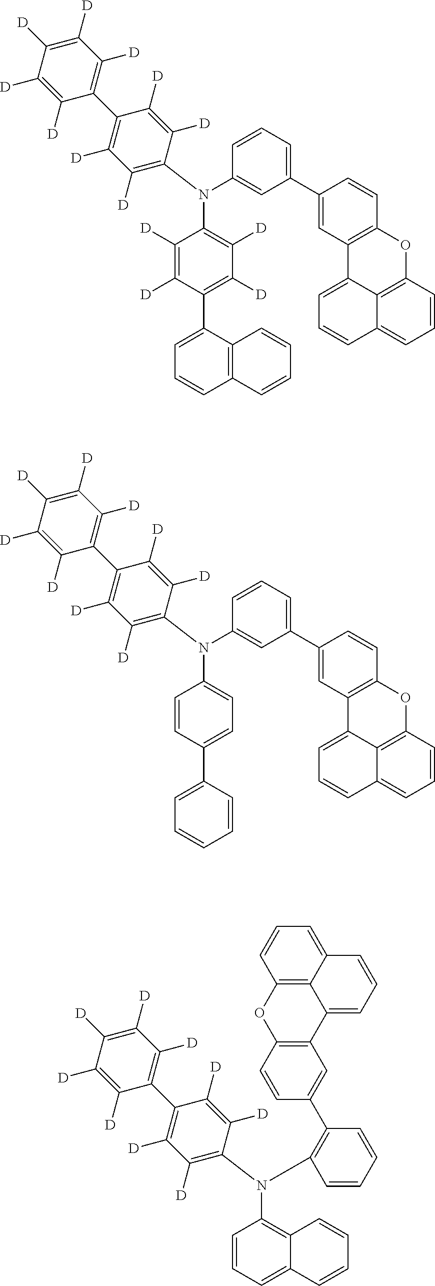 [SMILES: [2H]c1c([2H])c([2H])c(-c2c([2H])c([2H])c(N(c3cccc(-c4ccc5c(c4)-c4cccc6cccc(c46)O5)c3)c3c([2H])c([2H])c(-c4cccc5ccccc45)c([2H])c3[2H])c([2H])c2[2H])c([2H])c1[2H] ; [2H]c1c([2H])c([2H])c(-c2c([2H])c([2H])c(N(c3ccc(-c4ccccc4)cc3)c3cccc(-c4ccc5c(c4)-c4cccc6cccc(c46)O5)c3)c([2H])c2[2H])c([2H])c1[2H] ; [2H]c1c([2H])c([2H])c(-c2c([2H])c([2H])c(N(c3ccccc3-c3ccc4c(c3)-c3cccc5cccc(c35)O4)c3cccc4ccccc34)c([2H])c2[2H])c([2H])c1[2H]]

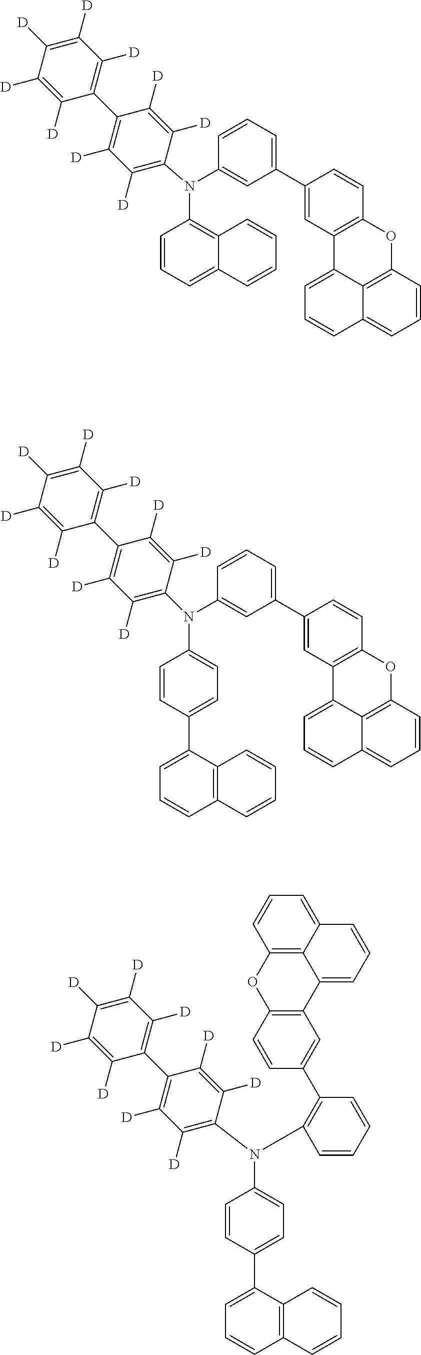 [SMILES: [2H]c1c([2H])c([2H])c(-c2c([2H])c([2H])c(N(c3cccc(-c4ccc5c(c4)-c4cccc6cccc(c46)O5)c3)c3cccc4ccccc34)c([2H])c2[2H])c([2H])c1[2H] ; [2H]c1c([2H])c([2H])c(-c2c([2H])c([2H])c(N(c3ccc(-c4cccc5ccccc45)cc3)c3cccc(-c4ccc5c(c4)-c4cccc6cccc(c46)O5)c3)c([2H])c2[2H])c([2H])c1[2H] ; [2H]c1c([2H])c([2H])c(-c2c([2H])c([2H])c(N(c3ccc(-c4cccc5ccccc45)cc3)c3ccccc3-c3ccc4c(c3)-c3cccc5cccc(c35)O4)c([2H])c2[2H])c([2H])c1[2H]]

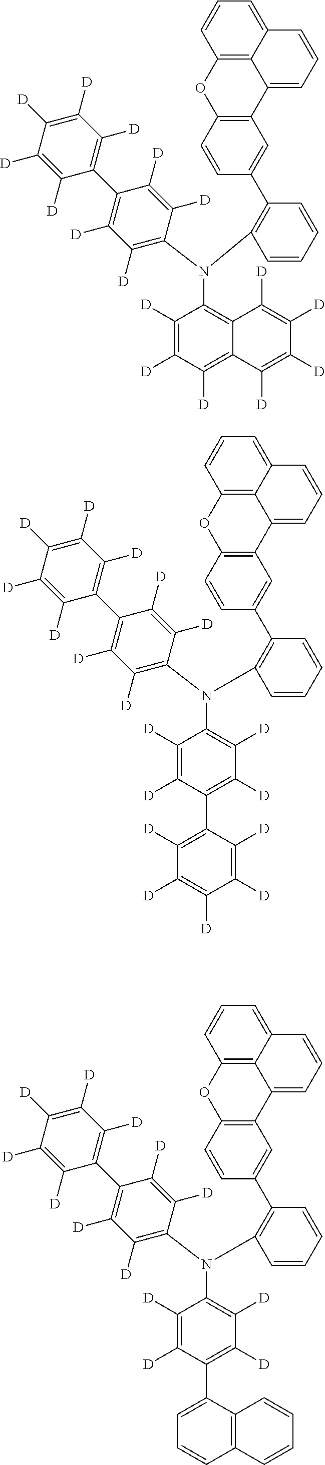

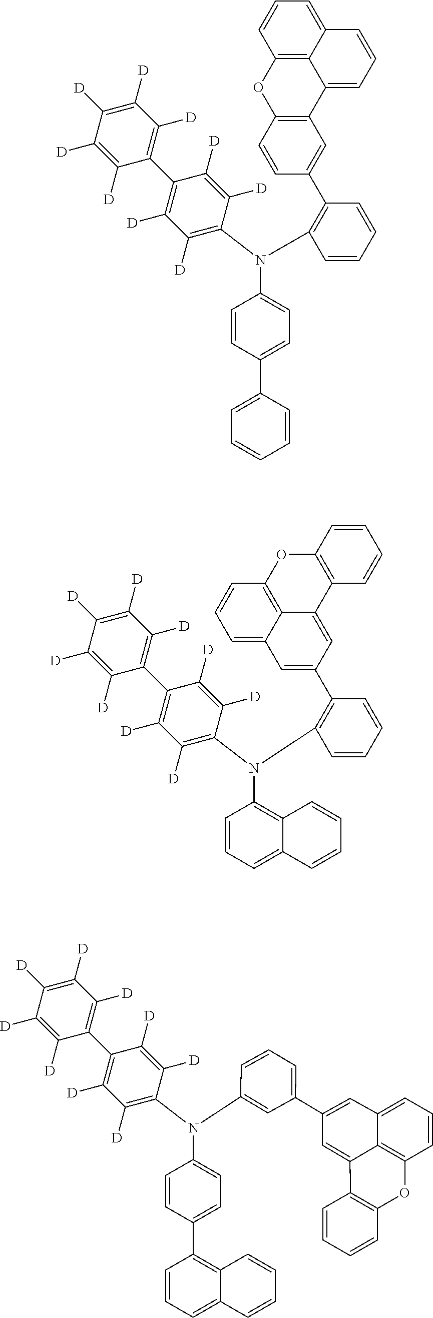 [SMILES: [2H]c1c([2H])c([2H])c(-c2c([2H])c([2H])c(N(c3ccc(-c4ccccc4)cc3)c3ccccc3-c3ccc4c(c3)-c3cccc5cccc(c35)O4)c([2H])c2[2H])c([2H])c1[2H] ; [2H]c1c([2H])c([2H])c(-c2c([2H])c([2H])c(N(c3ccccc3-c3cc4c5c(cccc5c3)Oc3ccccc3-4)c3cccc4ccccc34)c([2H])c2[2H])c([2H])c1[2H] ; [2H]c1c([2H])c([2H])c(-c2c([2H])c([2H])c(N(c3ccc(-c4cccc5ccccc45)cc3)c3cccc(-c4cc5c6c(cccc6c4)Oc4ccccc4-5)c3)c([2H])c2[2H])c([2H])c1[2H]]

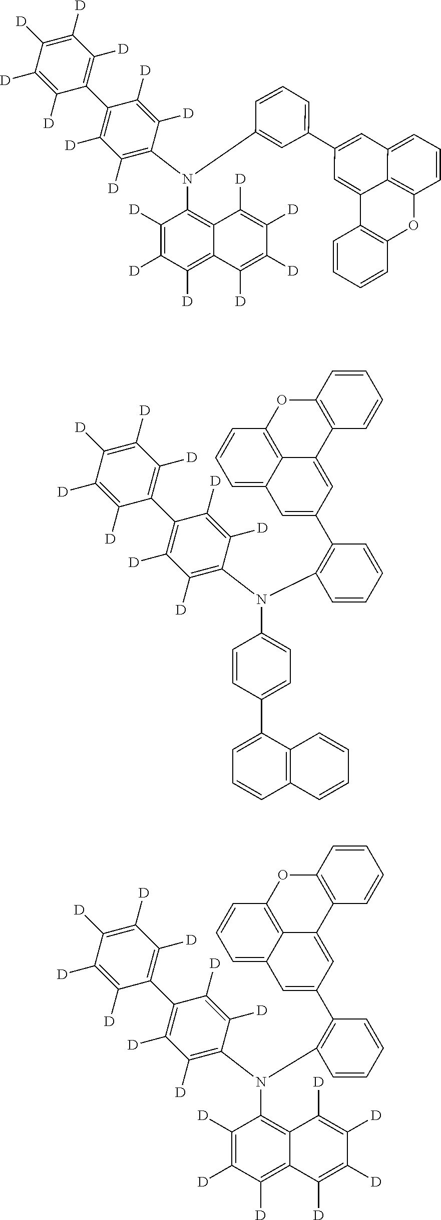 [SMILES: [2H]c1c([2H])c([2H])c(-c2c([2H])c([2H])c(N(c3cccc(-c4cc5c6c(cccc6c4)Oc4ccccc4-5)c3)c3c([2H])c([2H])c([2H])c4c([2H])c([2H])c([2H])c([2H])c34)c([2H])c2[2H])c([2H])c1[2H] ; [2H]c1c([2H])c([2H])c(-c2c([2H])c([2H])c(N(c3ccc(-c4cccc5ccccc45)cc3)c3ccccc3-c3cc4c5c(cccc5c3)Oc3ccccc3-4)c([2H])c2[2H])c([2H])c1[2H] ; [2H]c1c([2H])c([2H])c(-c2c([2H])c([2H])c(N(c3ccccc3-c3cc4c5c(cccc5c3)Oc3ccccc3-4)c3c([2H])c([2H])c([2H])c4c([2H])c([2H])c([2H])c([2H])c34)c([2H])c2[2H])c([2H])c1[2H]]

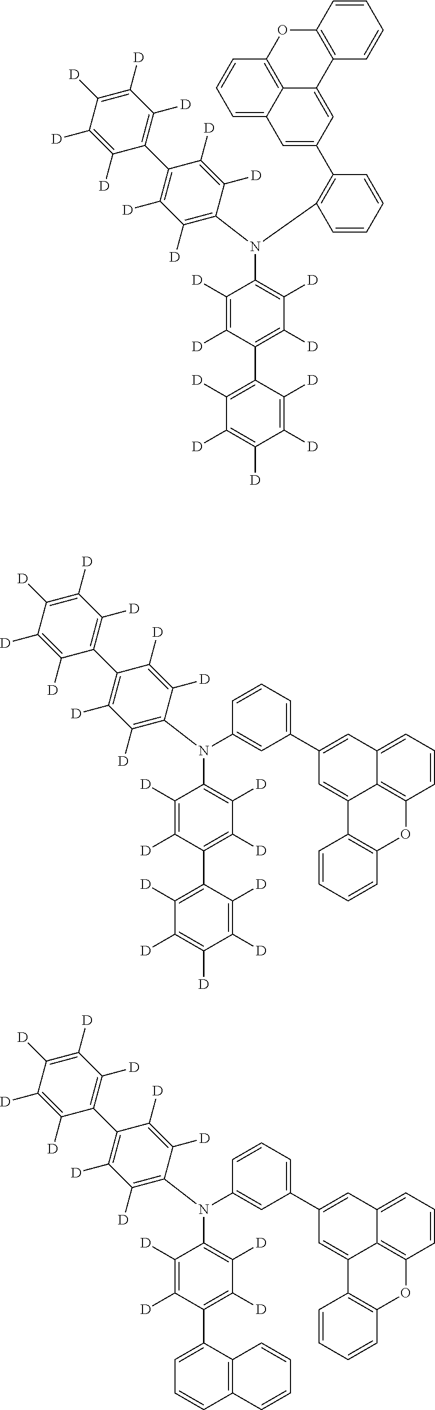

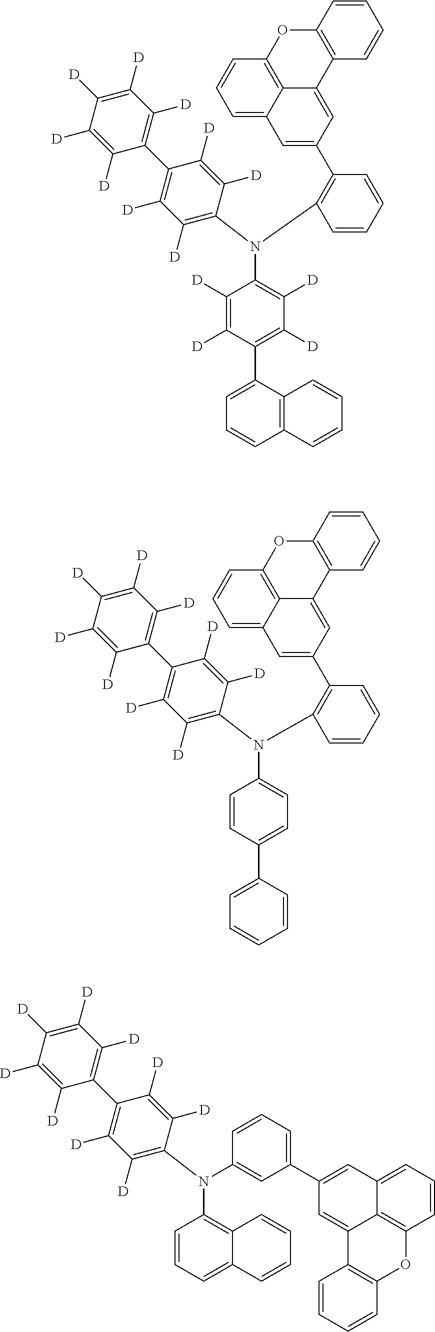 [SMILES: [2H]c1c([2H])c([2H])c(-c2c([2H])c([2H])c(N(c3ccccc3-c3cc4c5c(cccc5c3)Oc3ccccc3-4)c3c([2H])c([2H])c(-c4cccc5ccccc45)c([2H])c3[2H])c([2H])c2[2H])c([2H])c1[2H] ; [2H]c1c([2H])c([2H])c(-c2c([2H])c([2H])c(N(c3ccc(-c4ccccc4)cc3)c3ccccc3-c3cc4c5c(cccc5c3)Oc3ccccc3-4)c([2H])c2[2H])c([2H])c1[2H] ; [2H]c1c([2H])c([2H])c(-c2c([2H])c([2H])c(N(c3cccc(-c4cc5c6c(cccc6c4)Oc4ccccc4-5)c3)c3cccc4ccccc34)c([2H])c2[2H])c([2H])c1[2H]]

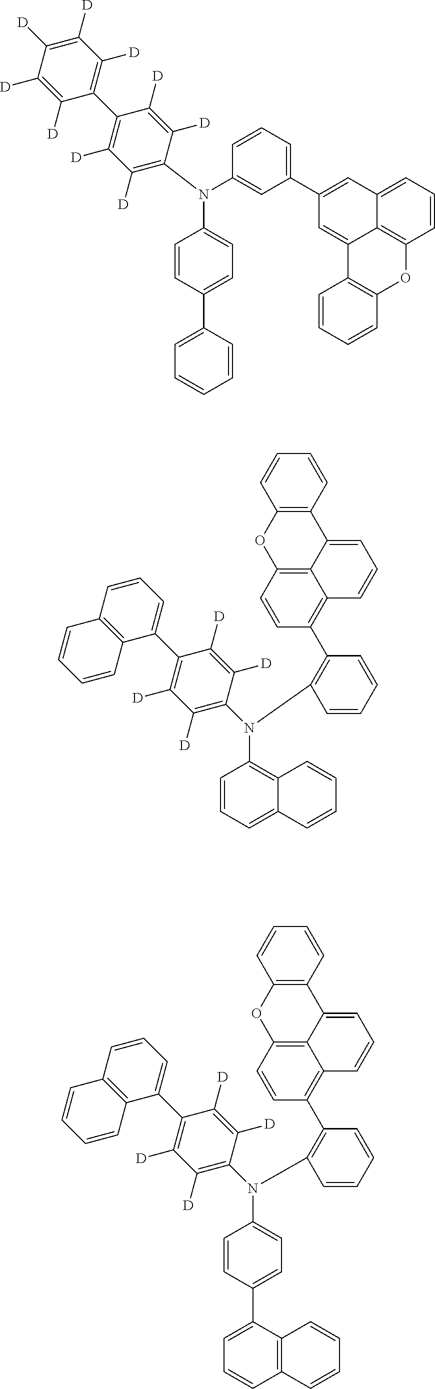 [SMILES: [2H]c1c([2H])c([2H])c(-c2c([2H])c([2H])c(N(c3ccc(-c4ccccc4)cc3)c3cccc(-c4cc5c6c(cccc6c4)Oc4ccccc4-5)c3)c([2H])c2[2H])c([2H])c1[2H] ; [2H]c1c([2H])c(N(c2ccccc2-c2ccc3c4c(cccc24)-c2ccccc2O3)c2cccc3ccccc23)c([2H])c([2H])c1-c1cccc2ccccc12 ; [2H]c1c([2H])c(N(c2ccc(-c3cccc4ccccc34)cc2)c2ccccc2-c2ccc3c4c(cccc24)-c2ccccc2O3)c([2H])c([2H])c1-c1cccc2ccccc12]

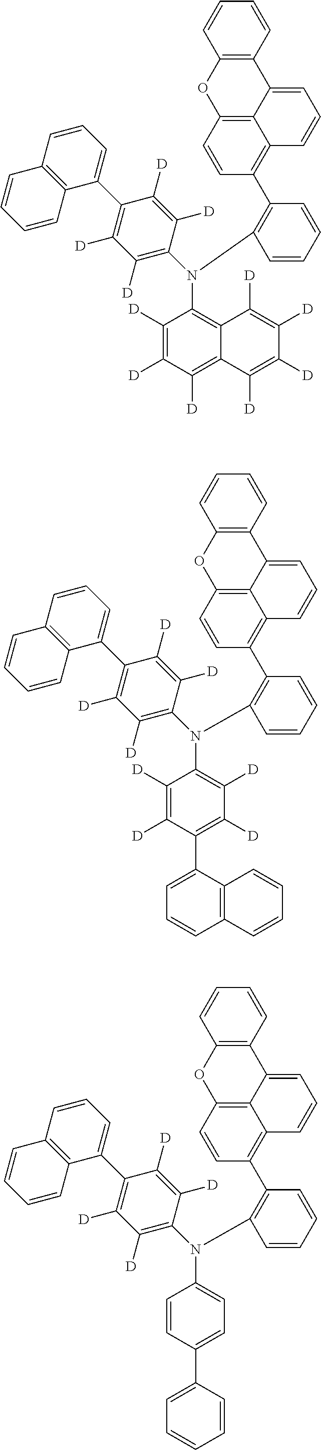 [SMILES: [2H]c1c([2H])c(N(c2ccccc2-c2ccc3c4c(cccc24)-c2ccccc2O3)c2c([2H])c([2H])c([2H])c3c([2H])c([2H])c([2H])c([2H])c23)c([2H])c([2H])c1-c1cccc2ccccc12 ; [2H]c1c([2H])c(N(c2ccccc2-c2ccc3c4c(cccc24)-c2ccccc2O3)c2c([2H])c([2H])c(-c3cccc4ccccc34)c([2H])c2[2H])c([2H])c([2H])c1-c1cccc2ccccc12 ; [2H]c1c([2H])c(N(c2ccc(-c3ccccc3)cc2)c2ccccc2-c2ccc3c4c(cccc24)-c2ccccc2O3)c([2H])c([2H])c1-c1cccc2ccccc12]

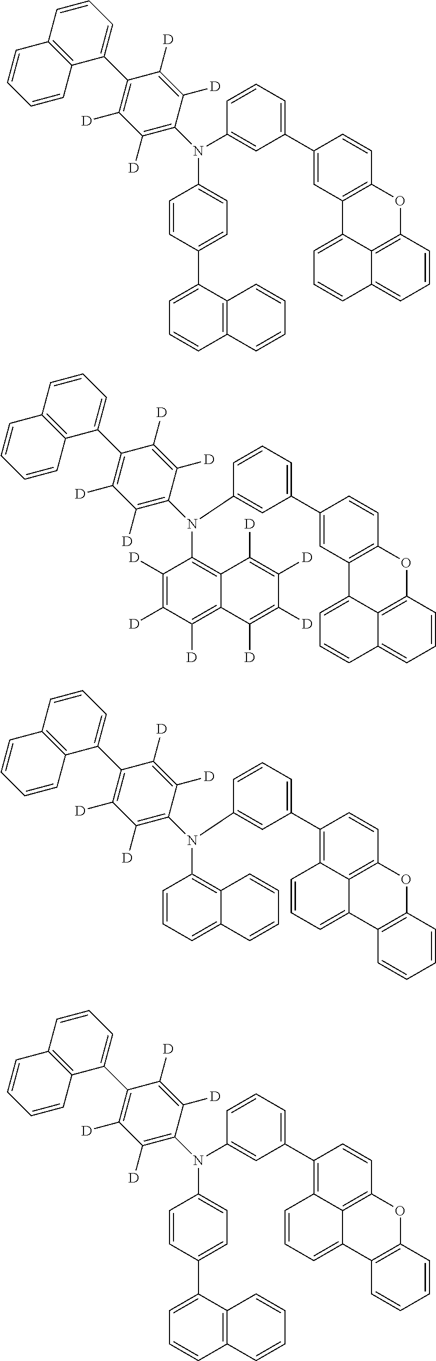 [SMILES: [2H]c1c([2H])c(N(c2ccc(-c3cccc4ccccc34)cc2)c2cccc(-c3ccc4c(c3)-c3cccc5cccc(c35)O4)c2)c([2H])c([2H])c1-c1cccc2ccccc12 ; [2H]c1c([2H])c(N(c2cccc(-c3ccc4c(c3)-c3cccc5cccc(c35)O4)c2)c2c([2H])c([2H])c([2H])c3c([2H])c([2H])c([2H])c([2H])c23)c([2H])c([2H])c1-c1cccc2ccccc12 ; [2H]c1c([2H])c(N(c2cccc(-c3ccc4c5c(cccc35)-c3ccccc3O4)c2)c2cccc3ccccc23)c([2H])c([2H])c1-c1cccc2ccccc12 ; [2H]c1c([2H])c(N(c2ccc(-c3cccc4ccccc34)cc2)c2cccc(-c3ccc4c5c(cccc35)-c3ccccc3O4)c2)c([2H])c([2H])c1-c1cccc2ccccc12]

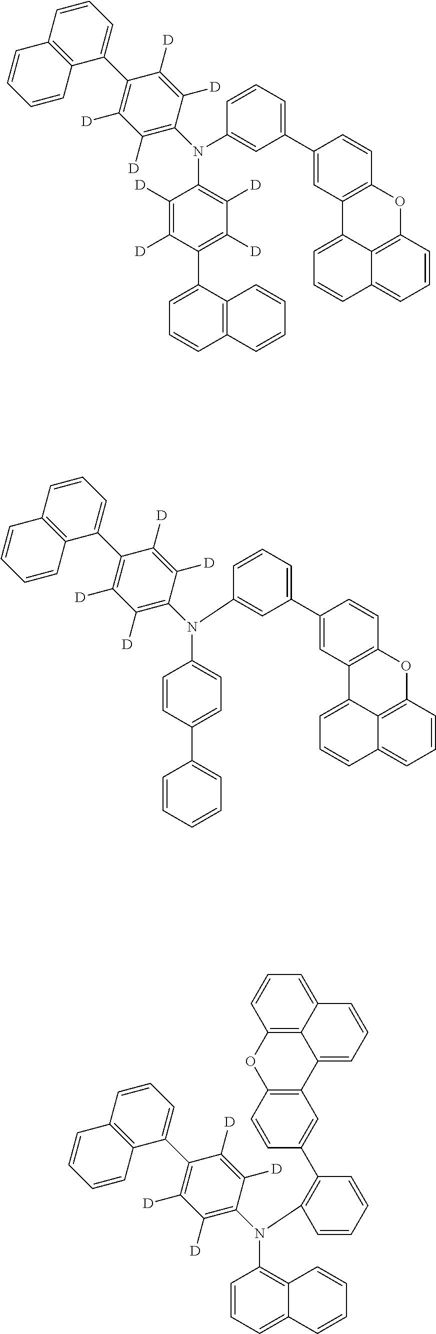 [SMILES: [2H]c1c([2H])c(N(c2cccc(-c3ccc4c(c3)-c3cccc5cccc(c35)O4)c2)c2c([2H])c([2H])c(-c3cccc4ccccc34)c([2H])c2[2H])c([2H])c([2H])c1-c1cccc2ccccc12 ; [2H]c1c([2H])c(N(c2ccc(-c3ccccc3)cc2)c2cccc(-c3ccc4c(c3)-c3cccc5cccc(c35)O4)c2)c([2H])c([2H])c1-c1cccc2ccccc12 ; [2H]c1c([2H])c(N(c2ccccc2-c2ccc3c(c2)-c2cccc4cccc(c24)O3)c2cccc3ccccc23)c([2H])c([2H])c1-c1cccc2ccccc12]

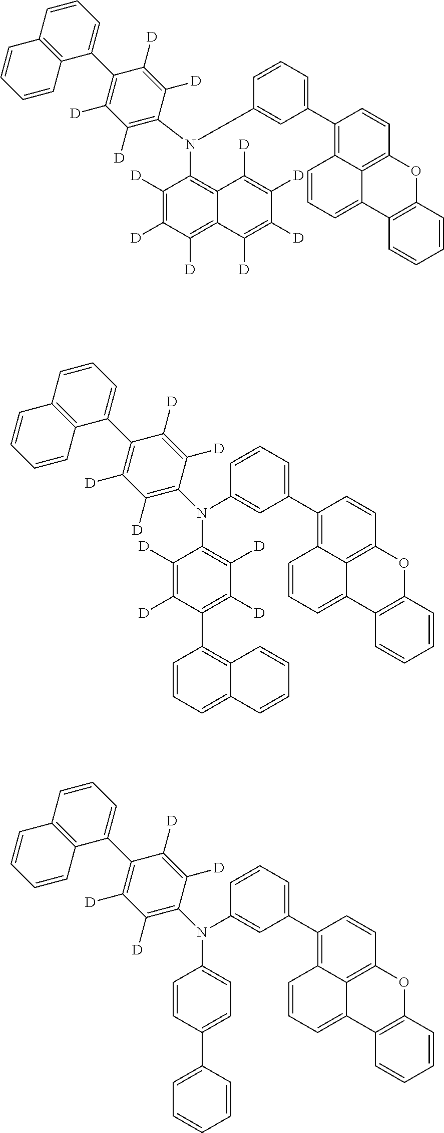 [SMILES: [2H]c1c([2H])c(N(c2cccc(-c3ccc4c5c(cccc35)-c3ccccc3O4)c2)c2c([2H])c([2H])c([2H])c3c([2H])c([2H])c([2H])c([2H])c23)c([2H])c([2H])c1-c1cccc2ccccc12 ; [2H]c1c([2H])c(N(c2cccc(-c3ccc4c5c(cccc35)-c3ccccc3O4)c2)c2c([2H])c([2H])c(-c3cccc4ccccc34)c([2H])c2[2H])c([2H])c([2H])c1-c1cccc2ccccc12 ; [2H]c1c([2H])c(N(c2ccc(-c3ccccc3)cc2)c2cccc(-c3ccc4c5c(cccc35)-c3ccccc3O4)c2)c([2H])c([2H])c1-c1cccc2ccccc12]

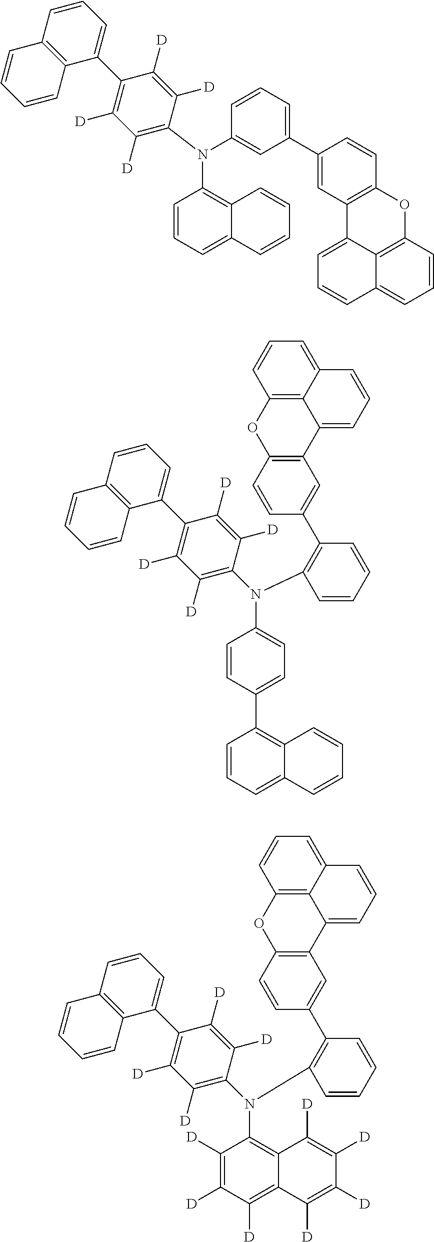

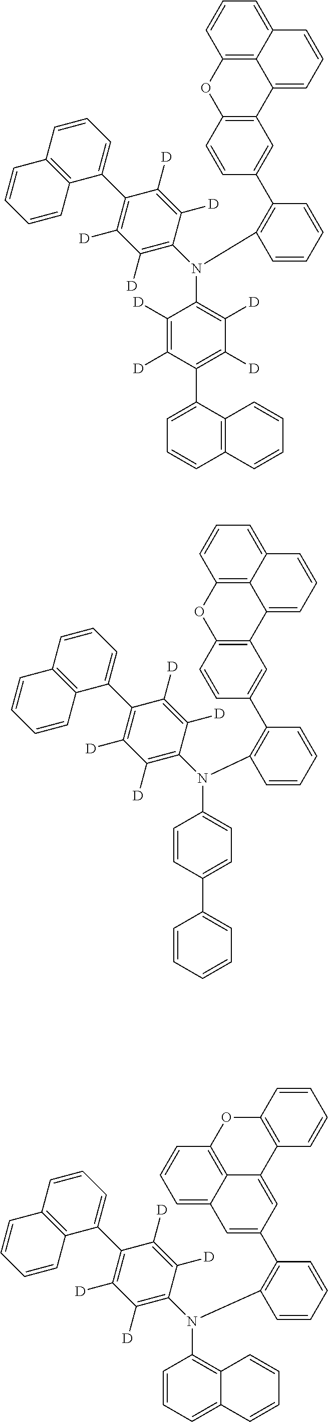 [SMILES: [2H]c1c([2H])c(N(c2ccccc2-c2ccc3c(c2)-c2cccc4cccc(c24)O3)c2c([2H])c([2H])c(-c3cccc4ccccc34)c([2H])c2[2H])c([2H])c([2H])c1-c1cccc2ccccc12 ; [2H]c1c([2H])c(N(c2ccc(-c3ccccc3)cc2)c2ccccc2-c2ccc3c(c2)-c2cccc4cccc(c24)O3)c([2H])c([2H])c1-c1cccc2ccccc12 ; [2H]c1c([2H])c(N(c2ccccc2-c2cc3c4c(cccc4c2)Oc2ccccc2-3)c2cccc3ccccc23)c([2H])c([2H])c1-c1cccc2ccccc12]

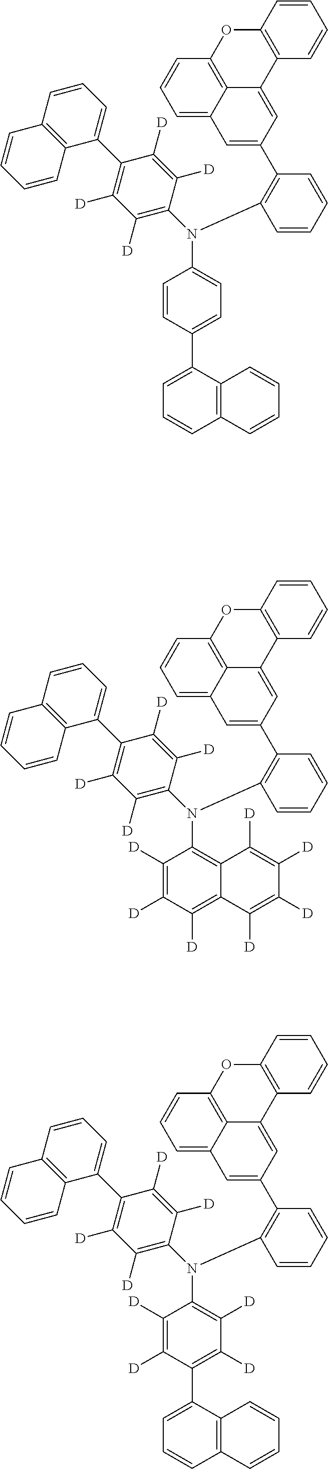 [SMILES: [2H]c1c([2H])c(N(c2ccc(-c3cccc4ccccc34)cc2)c2ccccc2-c2cc3c4c(cccc4c2)Oc2ccccc2-3)c([2H])c([2H])c1-c1cccc2ccccc12 ; [2H]c1c([2H])c(N(c2ccccc2-c2cc3c4c(cccc4c2)Oc2ccccc2-3)c2c([2H])c([2H])c([2H])c3c([2H])c([2H])c([2H])c([2H])c23)c([2H])c([2H])c1-c1cccc2ccccc12 ; [2H]c1c([2H])c(N(c2ccccc2-c2cc3c4c(cccc4c2)Oc2ccccc2-3)c2c([2H])c([2H])c(-c3cccc4ccccc34)c([2H])c2[2H])c([2H])c([2H])c1-c1cccc2ccccc12]

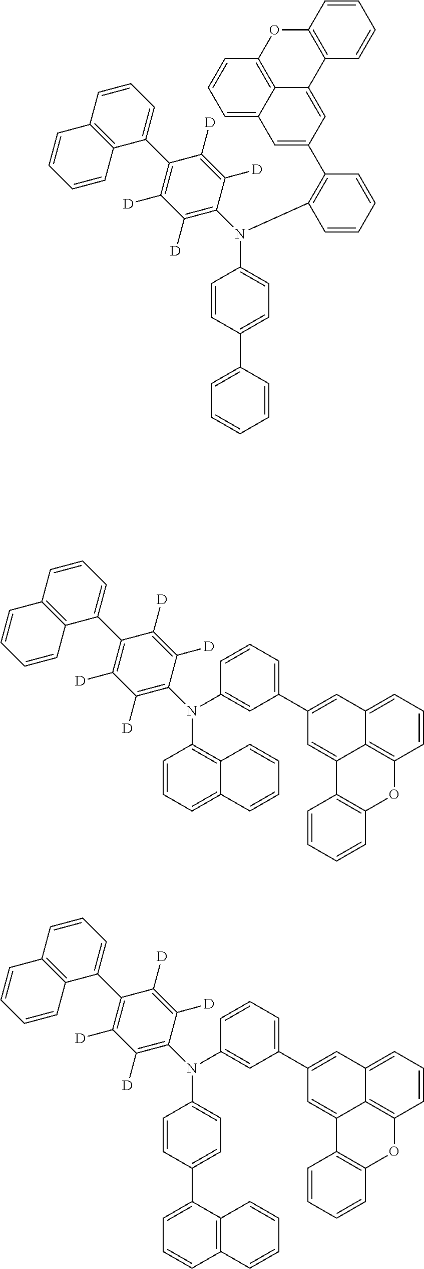 [SMILES: [2H]c1c([2H])c(N(c2ccc(-c3ccccc3)cc2)c2ccccc2-c2cc3c4c(cccc4c2)Oc2ccccc2-3)c([2H])c([2H])c1-c1cccc2ccccc12 ; [2H]c1c([2H])c(N(c2cccc(-c3cc4c5c(cccc5c3)Oc3ccccc3-4)c2)c2cccc3ccccc23)c([2H])c([2H])c1-c1cccc2ccccc12 ; [2H]c1c([2H])c(N(c2ccc(-c3cccc4ccccc34)cc2)c2cccc(-c3cc4c5c(cccc5c3)Oc3ccccc3-4)c2)c([2H])c([2H])c1-c1cccc2ccccc12]

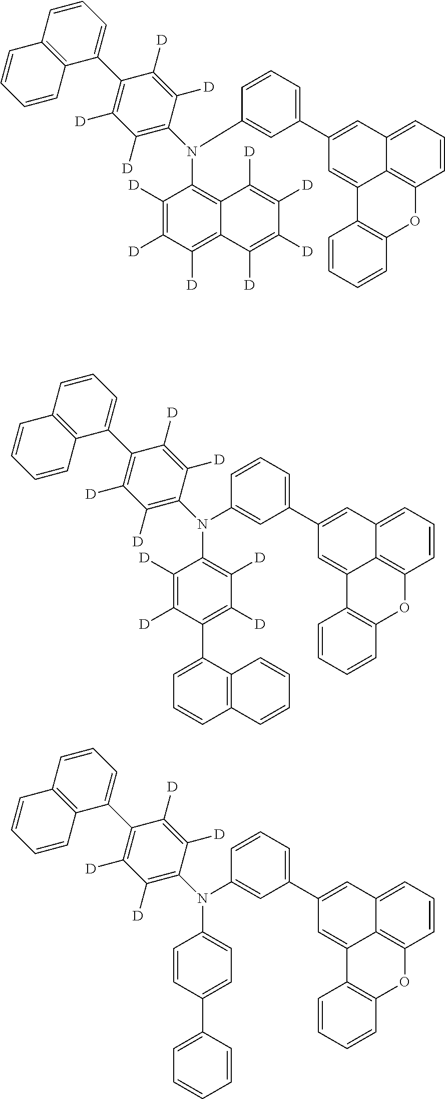 [SMILES: [2H]c1c([2H])c(N(c2cccc(-c3cc4c5c(cccc5c3)Oc3ccccc3-4)c2)c2c([2H])c([2H])c([2H])c3c([2H])c([2H])c([2H])c([2H])c23)c([2H])c([2H])c1-c1cccc2ccccc12 ; [2H]c1c([2H])c(N(c2cccc(-c3cc4c5c(cccc5c3)Oc3ccccc3-4)c2)c2c([2H])c([2H])c(-c3cccc4ccccc34)c([2H])c2[2H])c([2H])c([2H])c1-c1cccc2ccccc12 ; [2H]c1c([2H])c(N(c2ccc(-c3ccccc3)cc2)c2cccc(-c3cc4c5c(cccc5c3)Oc3ccccc3-4)c2)c([2H])c([2H])c1-c1cccc2ccccc12]

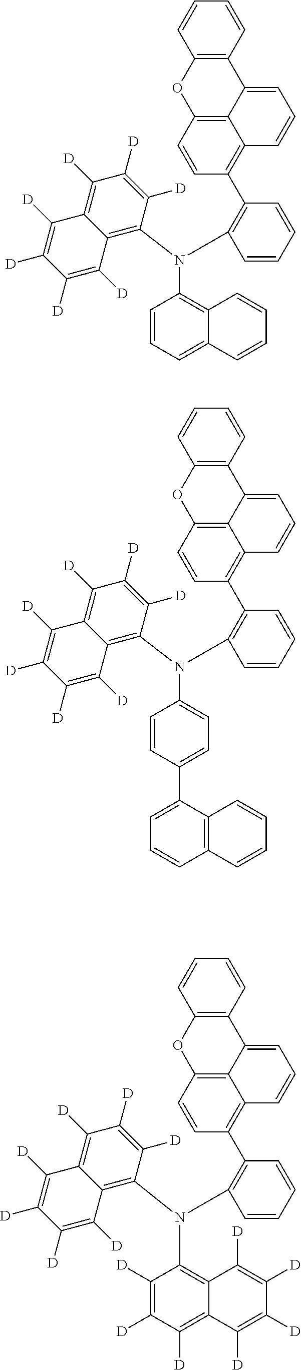 [SMILES: [2H]c1c([2H])c([2H])c2c(N(c3ccccc3-c3ccc4c5c(cccc35)-c3ccccc3O4)c3cccc4ccccc34)c([2H])c([2H])c([2H])c2c1[2H] ; [2H]c1c([2H])c([2H])c2c(N(c3ccc(-c4cccc5ccccc45)cc3)c3ccccc3-c3ccc4c5c(cccc35)-c3ccccc3O4)c([2H])c([2H])c([2H])c2c1[2H] ; [2H]c1c([2H])c([2H])c2c(N(c3ccccc3-c3ccc4c5c(cccc35)-c3ccccc3O4)c3c([2H])c([2H])c([2H])c4c([2H])c([2H])c([2H])c([2H])c34)c([2H])c([2H])c([2H])c2c1[2H]]

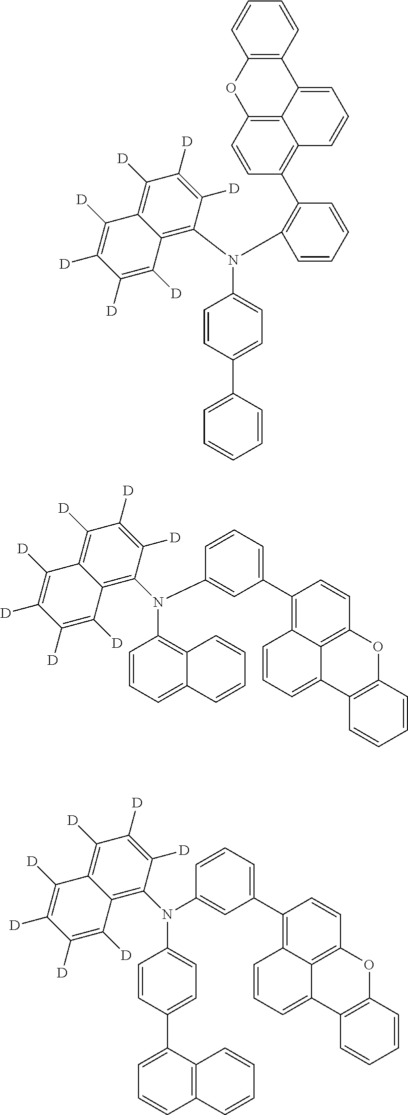 [SMILES: [2H]c1c([2H])c([2H])c2c(N(c3ccc(-c4ccccc4)cc3)c3ccccc3-c3ccc4c5c(cccc35)-c3ccccc3O4)c([2H])c([2H])c([2H])c2c1[2H] ; [2H]c1c([2H])c([2H])c2c(N(c3cccc(-c4ccc5c6c(cccc46)-c4ccccc4O5)c3)c3cccc4ccccc34)c([2H])c([2H])c([2H])c2c1[2H] ; [2H]c1c([2H])c([2H])c2c(N(c3ccc(-c4cccc5ccccc45)cc3)c3cccc(-c4ccc5c6c(cccc46)-c4ccccc4O5)c3)c([2H])c([2H])c([2H])c2c1[2H]]

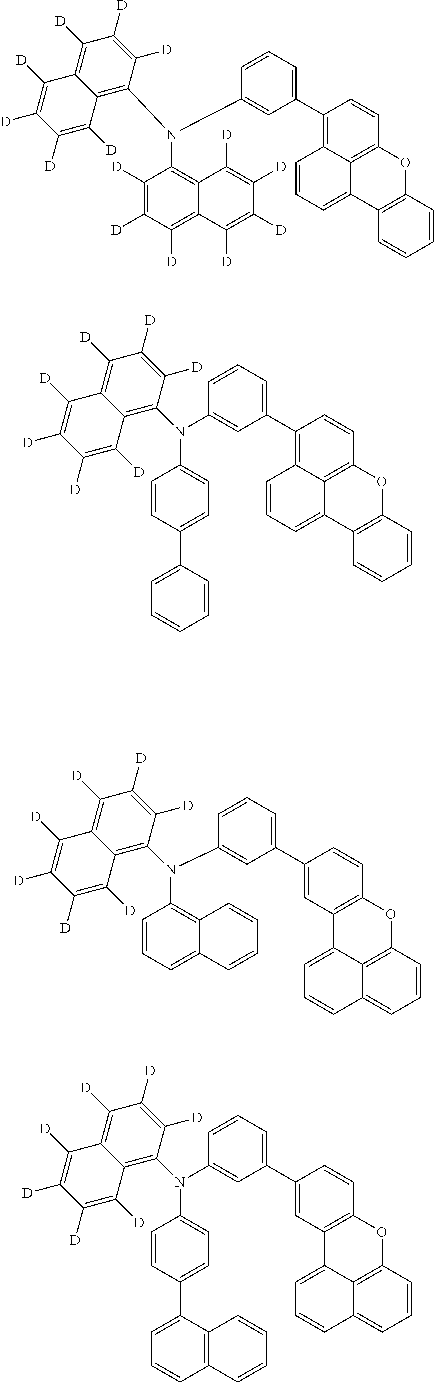

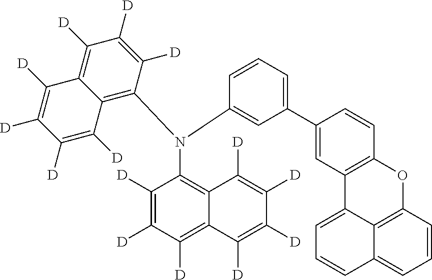 [SMILES: [2H]c1c([2H])c([2H])c2c(N(c3cccc(-c4ccc5c(c4)-c4cccc6cccc(c46)O5)c3)c3c([2H])c([2H])c([2H])c4c([2H])c([2H])c([2H])c([2H])c34)c([2H])c([2H])c([2H])c2c1[2H]]

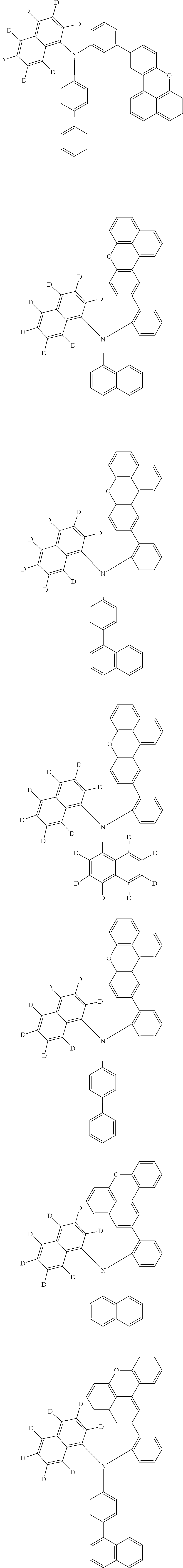

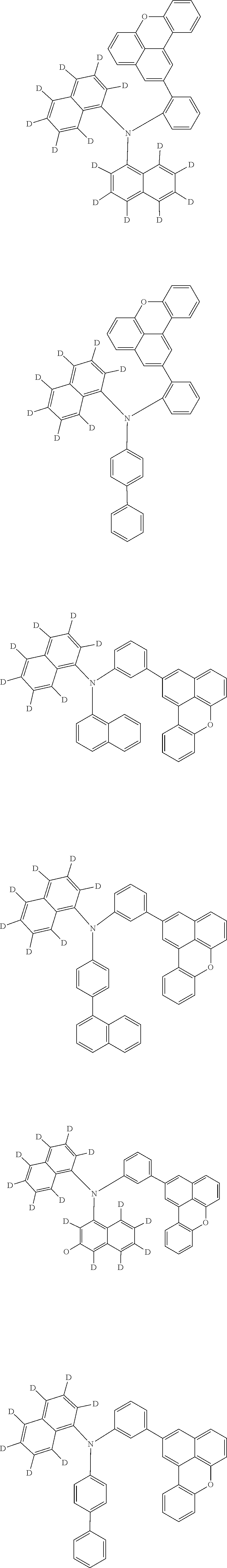

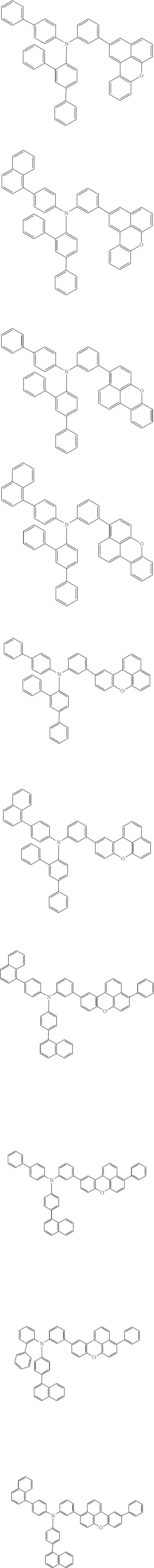

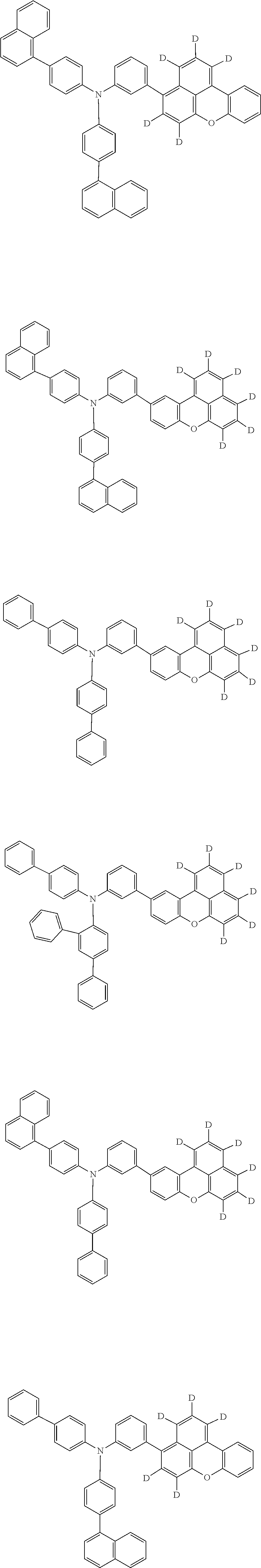

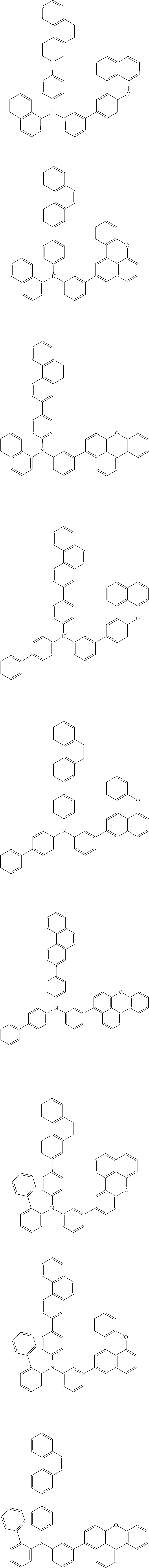

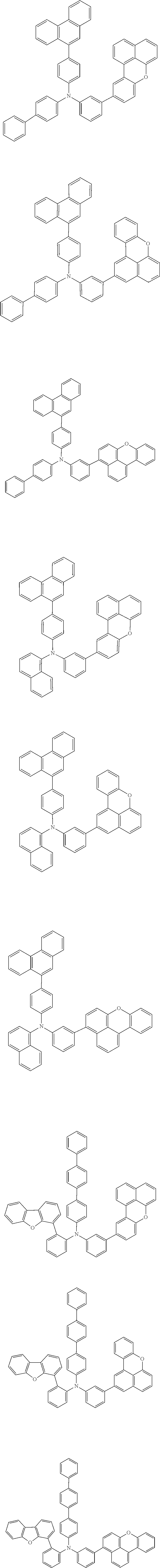

### Organic EL Device Material

An organic EL device material according to one aspect of the present invention includes the inventive compound. The content of the inventive compound in the organic EL device material is 1% by mass or more (including 100%), preferably 10% by mass or more (including 100%), more preferably 50% by mass or more (including 100%), still more preferably 80% by mass or more (including 100%), and particularly preferably 90% by mass or more (including 100%). The organic EL device material according to one aspect of the present invention is useful for production of an organic EL device.

In one embodiment of the present invention, the inventive compound is preferably a hole transporting layer material.

In one embodiment of the present invention, it is preferable that the organic EL device material further include a protium form of the inventive compound. The protium form is a compound in which all hydrogen atoms in the inventive compound are protium atoms.

The molar mixing ratio of the inventive compound to the protium form of the inventive compound (inventive compound:protium form) is preferably 10:90 to 90:10, more preferably 20:80 to 80:20, still more preferably 30:70 to 70:30, and particularly preferably 40:60 to 60:40.

An organic electroluminescent device material according to one aspect of the present invention is a hole transporting layer material.

The content of the inventive compound in the organic electroluminescent device material is preferably 1% by mass or more (including 100%), more preferably 10% by mass or more (including 100%), still more preferably 50% by mass or more (including 100%), further preferably 80% by mass or more (including 100%), and particularly preferably 90% by mass or more (including 100%).

### Organic EL Device

An organic EL device according to one aspect of the present invention includes an anode, a cathode, and an organic layer disposed between the anode and the cathode. The organic layer includes a light emitting layer, and at least one layer of the organic layer includes the inventive compound.

Examples of the organic layer including the inventive compound include, but are not limited to, a hole transporting band (a hole injecting layer, a hole transporting layer, an electron blocking layer, an exciton blocking layer, etc.) provided between the anode and the light emitting layer, a light emitting layer, a space layer, and an electron transporting band (an electron injecting layer, an electron transporting layer, a hole blocking layer, etc.) provided between the cathode and the light emitting layer. The inventive compound is preferably used as a material for a hole transporting band or a light emitting layer, more preferably as a material for a hole transporting band, still more preferably as a material for a hole injecting layer, a hole transporting layer, an electron blocking layer, or an exciton blocking layer, and particularly preferably as a material for a hole injecting layer or a hole transporting layer of a fluorescent or phosphorescent EL device.

The organic EL device according to one aspect of the present invention may be a fluorescent or phosphorescent single color light emitting device, may be a fluorescent/phosphorescent hybrid white light emitting device, may be a simple device including a single light emitting unit or a tandem device including a plurality of light emitting units, and is preferably a fluorescent light emitting device among them. Here, the “light emitting unit” refers to a minimum unit including an organic layer, in which at least one layer of the organic layer is a light emitting layer, and which emits light through recombination of injected holes and electrons.

Examples of a typical device configuration of the simple organic EL device include the following device configurations.

### (1) Anode/Light Emitting Unit/Cathode

In addition, the light emitting unit may be a multilayer light emitting unit having a plurality of phosphorescent light emitting layers and fluorescent light emitting layers. In this case, a space layer may be provided between the light emitting layers for the purpose of preventing excitons generated in the phosphorescent light emitting layer from diffusing into the fluorescent light emitting layer. Typical layer structures of the simple light emitting unit are shown below. The layers in parentheses are optional.

(a) (Hole injecting layer/) hole transporting layer/fluorescent light emitting layer/electron transporting layer (/electron injecting layer)

(b) (Hole injecting layer/) hole transporting layer/first fluorescent light emitting layer/second fluorescent light emitting layer/electron transporting layer (/electron injecting layer)

(c) (Hole injecting layer/) hole transporting layer/phosphorescent light emitting layer/space layer/fluorescent light emitting layer/electron transporting layer (/electron injecting layer)

(d) (Hole injecting layer/) hole transporting layer/first phosphorescent light emitting layer/second phosphorescent light emitting layer/space layer/fluorescent light emitting layer/electron transporting layer (/electron injecting layer)

(e) (Hole injecting layer/) hole transporting layer/phosphorescent light emitting layer/space layer/first fluorescent light emitting layer/second fluorescent light emitting layer/electron transporting layer (/electron injecting layer)

(f) (Hole injecting layer/) hole transporting layer/electron blocking layer/fluorescent light emitting layer/electron transporting layer (/electron injecting layer)

(g) (Hole injecting layer/hole transporting layer/exciton blocking layer/fluorescent light emitting layer/electron transporting layer (/electron injecting layer)

(h) (Hole injecting layer/) first hole transporting layer/second hole transporting layer/fluorescent light emitting layer/electron transporting layer (/electron injecting layer)

(i) (Hole injecting layer/) first hole transporting layer/second hole transporting layer/fluorescent light emitting layer/first electron transporting layer/second electron transporting layer (/electron injecting layer)

(j) (Hole injecting layer/) hole transporting layer/fluorescent light emitting layer/hole blocking layer/electron transporting layer (/electron injecting layer)

(k) (Hole injecting layer/) hole transporting layer/fluorescent light emitting layer/exciton blocking layer/electron transporting layer (/electron injecting layer)

Each phosphorescent light or fluorescent light emitting layer may exhibit a different emission color. Specifically, an exemplary layer structure in the light emitting unit (d) is (hole injecting layer/) hole transporting layer/first phosphorescent light emitting layer (red light emission)/second phosphorescent light emitting layer (green light emission)/space layer/fluorescent light emitting layer (blue light emission)/electron transporting layer.

An electron blocking layer may be provided, as appropriate, between each light emitting layer and the hole transporting layer or the space layer. In addition, a hole blocking layer may be provided, as appropriate, between each light emitting layer and the electron transporting layer. When the electron blocking layer or the hole blocking layer is provided, electrons or holes are confined within a light emitting layer, the recombination probability of charges in the light emitting layer is increased, and light emission efficiency can be improved.

A typical device configuration of the tandem organic EL device can include the following device structure.

### (2) Anode/First Light Emitting Unit/Intermediate Layer/Second Light Emitting Unit/Cathode

Here, the first light emitting unit and the second light emitting unit may be independently selected from the above-described light emitting units, for example.

The intermediate layer is generally referred to as an intermediate electrode, an intermediate conductive layer, a charge generating layer, an electron extracting layer, a connection layer, or an intermediate insulating layer, and a known material configuration that supplies electrons to the first light emitting unit and supplies holes to the second light emitting unit can be used.

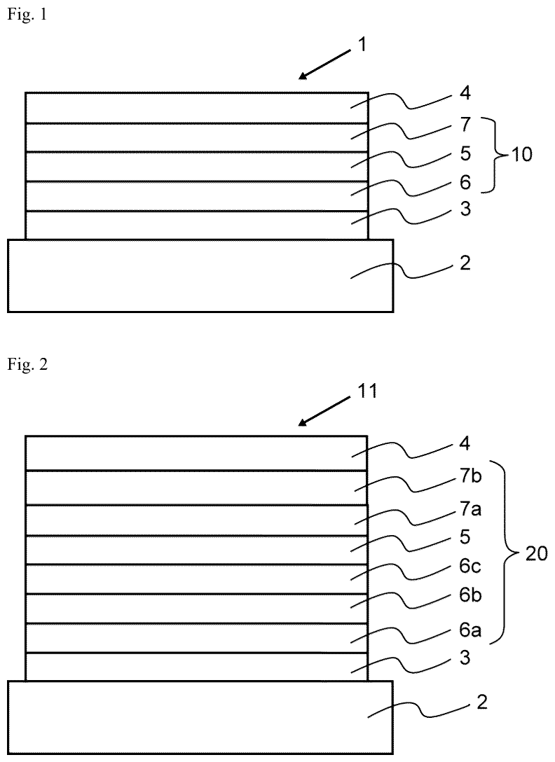

FIG. 1 is a schematic diagram illustrating an example of a structure of an organic EL device according to one embodiment of the present invention. An organic EL device 1 has a substrate 2, an anode 3, a cathode 4, and a light emitting unit 10 disposed between the anode 3 and the cathode 4. The light emitting unit 10 has a light emitting layer 5. A hole transporting band 6 (hole injecting layer, hole transporting layer, etc.) is provided between the light emitting layer 5 and the anode 3, and an electron transporting band 7 (electron injecting layer, electron transporting layer, etc.) is provided between the light emitting layer 5 and the cathode 4. An electron blocking layer (not shown) may be provided on the anode 3 side of the light emitting layer 5, and a hole blocking layer (not shown) may be provided on the cathode 4 side of the light emitting layer 5. Consequently, electrons and holes are confined within the light emitting layer 5, and the efficiency of exciton generation in the light emitting layer 5 can be further enhanced.

FIG. 2 is a schematic view illustrating another structure of the organic EL device according to one embodiment of the present invention. An organic EL device 11 has the substrate 2, the anode 3, the cathode 4, and a light emitting unit 20 disposed between the anode 3 and the cathode 4. The light emitting unit 20 has the light emitting layer 5. A hole transporting band disposed between the anode 3 and the light emitting layer 5 is formed from a hole injecting layer 6a, a first hole transporting layer 6b, and a second hole transporting layer 6c. In addition, an electron transporting band disposed between the light emitting layer 5 and the cathode 4 is formed from a first electron transporting layer 7a and a second electron transporting layer 7b.

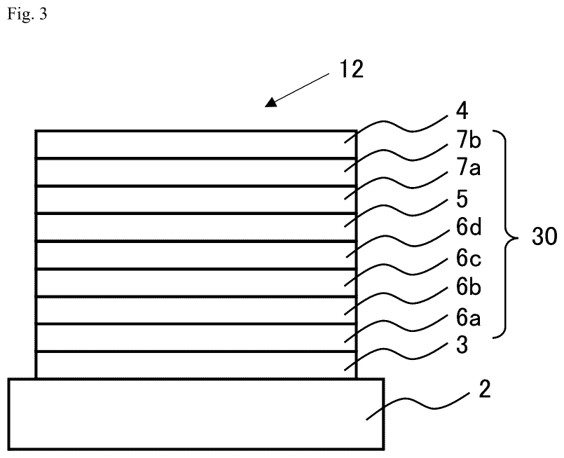

FIG. 3 is a schematic view illustrating still another structure of the organic EL device according to one embodiment of the present invention. An organic EL device 12 has the substrate 2, the anode 3, the cathode 4, and a light emitting unit 30 disposed between the anode 3 and the cathode 4. The light emitting unit 30 has the light emitting layer 5. A hole transporting band disposed between the anode 3 and the light emitting layer 5 is formed from the hole injecting layer 6a, the first hole transporting layer 6b, the second hole transporting layer 6c, and a third hole transporting layer 6d. In addition, an electron transporting band disposed between the light emitting layer 5 and the cathode 4 is formed from the first electron transporting layer 7a and the second electron transporting layer 7b.

In FIG. 1 to FIG. 3, the light emitting layer 5 includes at least one light emitting layer. The light emitting layer 5 may be a single layer and may include multiple layers (for example, a plurality of light emitting layers, a plurality of light emitting layers and a space layer).

In the present invention, a host combined with a fluorescent dopant material (fluorescent light emitting material) is referred to as a fluorescent host, and a host combined with a phosphorescent dopant material is referred to as a phosphorescent host. The fluorescent host and the phosphorescent host are not distinguished only by the molecular structures thereof. That is, the phosphorescent host means a material that forms a phosphorescent light emitting layer containing a phosphorescent dopant, and does not mean that the phosphorescent host cannot be used as a material for forming a fluorescent light emitting layer. The same applies to the fluorescent host.

### Substrate

The substrate is used as a support of the organic EL device. As the substrate, a plate of glass, quartz, or plastic can be used, for example. Alternatively, a flexible substrate may be used. Examples of the flexible substrate include plastic substrates made of polycarbonate, polyarylate, polyethersulfone, polypropylene, polyester, polyvinyl fluoride, and polyvinyl chloride. In addition, an inorganic vapor deposition film can also be used.

### Anode

As the anode formed above the substrate, a metal, an alloy, an electrically conductive compound, or a mixture thereof having a high work function (specifically, a work function of 4.0 eV or more) is preferably used. Specific examples thereof include indium oxide-tin oxide (ITO: indium tin oxide), indium oxide-tin oxide containing silicon or silicon oxide, indium oxide-zinc oxide, indium oxide containing tungsten oxide and zinc oxide, and graphene. Other examples include gold (Au), platinum (Pt), nickel (Ni), tungsten (W), chromium (Cr), molybdenum (Mo), iron (Fe), cobalt (Co), copper (Cu), palladium (Pd), titanium (Ti), and nitrides of the above metals (for example, titanium nitride).

These materials are usually formed into a film using a sputtering method. For example, indium oxide-zinc oxide can be formed by a sputtering method using a target in which 1 to 10 wt % of zinc oxide is added to indium oxide, and indium oxide containing tungsten oxide and zinc oxide can be formed by a sputtering method using a target in which 0.5 to 5 wt % of tungsten oxide and 0.1 to 1 wt % of zinc oxide are added to indium oxide. Besides, a vacuum deposition method, a coating method, an inkjet method, and a spin coating method may be used.

### Hole Transporting Band

As described above, the organic layer may include a hole transporting band between the anode and the light emitting layer. The hole transporting band is composed of a hole injecting layer, hole transporting layer, an electron blocking layer, and the like. The hole transporting band preferably includes the inventive compound. It is preferable that at least one layer of these layers constituting the hole transporting layer include the inventive compound, and in particular, it is more preferable that the inventive compound be included in the hole transporting layer.

Since the hole injecting layer formed in contact with the anode is formed by using a material which can easily inject holes regardless of the work function of the anode, a material generally used as an electrode material (for example, metals, alloys, electrically conductive compounds, and mixtures thereof, and elements belonging to Group 1 or 2 of the periodic table of the elements) can be used.

An element belonging to Group 1 or 2 of the periodic table of the elements, which is a material having a low work function, that is, alkali metals such as lithium (Li) and cesium (Cs), alkaline earth metals such as magnesium (Mg), calcium (Ca), and strontium (Sr), and alloys containing these elements (for example, MgAg and AlLi), rare earth metals such as europium (Eu) and ytterbium (Yb), and alloys containing these elements can also be used. When the anode is formed using an alkali metal, an alkaline earth metal, or an alloy thereof, a vacuum deposition method or a sputtering method can be used. Further, in a case of using a silver paste or the like, a coating method or an inkjet method can be used.

### Hole Injecting Layer

The hole injecting layer is a layer including a material having high hole injecting performance (hole injecting material), and is formed between the anode and the light emitting layer or between the hole transporting layer and the anode when present.

As the hole injecting material other than the inventive compound, a molybdenum oxide, a titanium oxide, a vanadium oxide, a rhenium oxide, a ruthenium oxide, a chromium oxide, a zirconium oxide, a hafnium oxide, a tantalum oxide, a silver oxide, a tungsten oxide, a manganese oxide, or the like can be used.

The hole injecting layer material also includes aromatic amine compounds such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), and 3-[N-(1-naphthyl)-N-(9-phenylcarbazol-3-yl)amino]-9-phenylcarbazole (abbreviation: PCzPCN1), which are low molecular weight organic compounds.

A high molecular weight compound (such as an oligomer, a dendrimer, or a polymer) can also be used. Examples thereof include high molecular weight compounds such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide](abbreviation: PTPDMA), and poly[N,N′-bis(4-butylphenyl)-N,N′-bis(phenyl)benzidine](abbreviation: Poly-TPD). In addition, a high molecular weight compound to which an acid is added such as poly(3,4-ethylenedioxythiophene)/poly(styrenesulfonic acid) (PEDOT/PSS) or polyaniline/poly(styrenesulfonic acid) (PAni/PSS) can also be used.

Further, it is also preferable to use an acceptor material such as a hexaazatriphenylene (HAT) compound represented by the following formula (K).

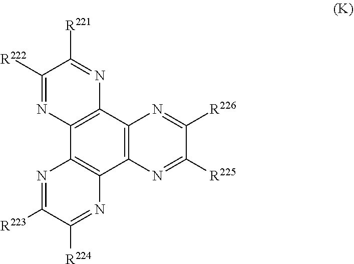

In the above formula, R²²¹ to R²²⁶ each independently represent a cyano group, —CONH₂, a carboxy group, or —COOR²²⁷ (R²²⁷ represents an alkyl group having 1 to 20 carbon atoms or a cycloalkyl group having 3 to 20 carbon atoms). In addition, adjacent two moieties selected from R²²¹ and R²²², R²²³ and R²²⁴, and R²²⁵ and R²²⁶ may be bonded to each other to form a group represented by —CO—O—CO—.

Examples of R²²⁷ include a methyl group, an ethyl group, a n-propyl group, an isopropyl group, a n-butyl group, an isobutyl group, a t-butyl group, a cyclopentyl group, and a cyclohexyl group.

### Hole Transporting Layer

The hole transporting layer is a layer including a material having high hole transporting performance (hole transporting material), and is formed between the anode and the light emitting layer or between the hole injecting layer and the light emitting layer when present. The inventive compound may be used singly or in combination with the following compound for the hole transporting layer.

The hole transporting layer may have a single layer structure or a multilayer structure including two or more layers. For example, the hole transporting layer may have a two-layer structure including a first hole transporting layer (on the anode side) and a second hole transporting layer (on the cathode side). That is, the hole transporting band may include the first hole transporting layer on the anode side and the second hole transporting layer on the cathode side. The hole transporting layer may have a three-layer structure including a first hole transporting layer, a second hole transporting layer, and a third hole transporting layer in this order from the anode side. That is, the third hole transporting layer may be disposed between the second hole transporting layer and the light emitting layer.

In one embodiment of the present invention, it is preferable that the hole transporting layer of the single layer structure be adjacent to the light emitting layer, and it is preferable that a hole transporting layer closest to the cathode in the multilayer structure, for example, the second hole transporting layer of the two-layer structure or the third hole transporting layer of the three-layer structure be adjacent to the light emitting layer. In another embodiment of the present invention, an electron blocking layer, which will be described later, may be interposed between the hole transporting layer and the light emitting layer of the single layer structure or between the hole transporting layer closest to the light emitting layer and the light emitting layer in the multilayer structure.

In one embodiment of the organic electroluminescent device according to the present invention, at least one of the first hole transporting layer and the second hole transporting layer includes the inventive compound. Specifically, in the hole transporting layer in the two-layer structure, the inventive compound may be included in one or both of the first hole transporting layer and the second hole transporting layer. In another embodiment, at least one of the first to third hole transporting layers includes the inventive compound. Specifically, in the hole transporting layer in the three-layer structure, the inventive compound may be included in only one of the first to third hole transporting layers, may be included in only two of the first to third hole transporting layers, or may be included in all of the first to third hole transporting layers.

In one embodiment of the present invention, the inventive compound is preferably included in the second hole transporting layer. Specifically, it is preferable that the inventive compound be included only in the second hole transporting layer, or the inventive compound be included in the first hole transporting layer and the second hole transporting layer.

In one embodiment of the present invention, the inventive compound included in one or both of the first hole transporting layer and the second hole transporting layer or the inventive compound included in at least one or more of the first to third hole transporting layers is preferably a protium form from the viewpoint of manufacturing costs.

The protium form is the inventive compound in which all hydrogen atoms in the inventive compound are protium atoms.

Accordingly, the present invention includes an organic EL device in which at least one or both of the first hole transporting layer and the second hole transporting layer or at least one or more of the first to third hole transporting layers include the inventive compound substantially composed only of the protium form. The “inventive compound composed only of the protium form” means that the content ratio of the protium form based on the total amount of the inventive compound is 90 mol % or more, preferably 95 mol % or more, and more preferably 99 mol % or more (each including 100%).

An aromatic amine compound, a carbazole derivative, or an anthracene derivative can be used as the hole transporting layer material other than the inventive compound, for example.

Examples of the aromatic amine compound include 4,4′-bis[N-(1-naphthyl)-N-phenylamino]biphenyl (abbreviation: NPB) and N,N′-bis(3-methylphenyl)-N,N′-diphenyl-[1,1′-biphenyl]-4,4′-diamine (abbreviation: TPD), 4-phenyl-4′-(9-phenylfluoren-9-yl)triphenylamine (abbreviation: BAFLP), 4,4′-bis[N-(9,9-dimethylfluoren-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluoren-2-yl)-N-phenylamino]biphenyl (abbreviation: BSPB). The compound has hole mobility of 106 cm²/Vs or more.

Examples of the carbazole derivative include 4,4′-di(9-carbazolyl)biphenyl (abbreviation: CBP), 9-[4-(9-carbazolyl)phenyl]-10-phenylanthracene (abbreviation: CzPA), and 9-phenyl-3-[4-(10-phenyl-9-anthryl)phenyl]-9H-carbazole (abbreviation: PCzPA).

Examples of the anthracene derivative include 2-t-butyl-9,10-di(2-naphthyl)anthracene (abbreviation: t-BuDNA), 9,10-di(2-naphthyl)anthracene (abbreviation: DNA), and 9,10-diphenylanthracene (abbreviation: DPAnth).

A high molecular weight compound such as poly(N-vinylcarbazole) (abbreviation: PVK) or poly(4-vinyltriphenylamine) (abbreviation: PVTPA) can also be used.

However, a compound other than the compounds mentioned above may be used as long as it exhibits higher hole transporting performance compared with electron transporting performance.

In one embodiment of the organic EL device according to the present invention, the first hole transporting layer includes a compound represented by the following formula (21) or formula (22).

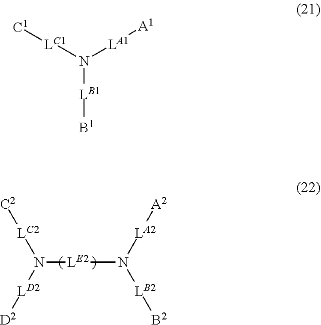

In the formula (21) and the formula (22),
L^{A1}, L^{B1}, L^{C1}, L^{A2}, L^{B2}, L^{C2}, and L^{D2} are each independently a single bond, a substituted or unsubstituted arylene group having 6 to 50 ring carbon atoms, or a substituted or unsubstituted divalent heterocyclic group having 5 to 50 ring atoms,
k is 1, 2, 3, or 4,
when k is 1, L^{E2} is a substituted or unsubstituted arylene group having 6 to 50 ring carbon atoms or a substituted or unsubstituted divalent heterocyclic group having 5 to 50 ring atoms,
when k is 2, 3, or 4, the groups each represented by L^{E2} are the same as or different from one another
when k is 2, 3, or 4, the groups each represented by L^{E2} are bonded to each other to form a substituted or unsubstituted monocyclic ring, are bonded to each other to form a substituted or unsubstituted condensed ring, or are not bonded to each other,
L^{E2} that forms neither the monocyclic ring nor the condensed ring is a substituted or unsubstituted arylene group having 6 to 50 ring carbon atoms or a substituted or unsubstituted divalent heterocyclic group having 5 to 50 ring atoms,
A¹, B¹, C¹, A², B², C², and D² are each independently a substituted or unsubstituted aryl group having 6 to 50 ring carbon atoms, a substituted or unsubstituted heterocyclic group having 5 to 50 ring atoms, or —Si(R′₉₀₁)(R′₉₀₂)(R′₉₀₃)
R′₉₀₁, R′₉₀₂, and R′₉₀₃ are each independently a substituted or unsubstituted aryl group having 6 to 50 ring carbon atoms,
when multiple groups each represented by R′₉₀₁ exist, the multiple groups each represented by R′₉₀₁ are the same as or different from one another,
when multiple groups each represented by R′₉₀₂ exist, the multiple groups each represented by R′₉₀₂ are the same as or different from one another, and
when multiple groups each represented by R′₉₀₃ exist, the multiple groups each represented by R′₉₀₃ are the same as or different from one another.
R₉₀₁ to R₉₀₇ are each independently a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted heterocyclic group having 5 to 50 ring atoms; and
when multiple groups each represented by R₉₀₁ exist, the multiple groups each represented by R₉₀₁ are the same as or different from one another,
when multiple groups each represented by R₉₀₂ exist, the multiple groups each represented by R₉₀₂ are the same as or different from one another,
when multiple groups each represented by R₉₀₃ exist, the multiple groups each represented by R₉₀₃ are the same as or different from one another,
when multiple groups each represented by R₉₀₄ exist, the multiple groups each represented by R₉₀₄ are the same as or different from one another,
when multiple groups each represented by R₉₀₅ exist, the multiple groups each represented by R₉₀₅ are the same as or different from one another,
when multiple groups each represented by R₉₀₆ exist, the multiple groups each represented by R₉₀₆ are the same as or different from one another, and
when multiple groups each represented by R₉₀₇ exist, the multiple groups each represented by R₉₀₇ are the same as or different from one another.

The first hole transporting layer may contain one kind of compounds represented by formula (21) and formula (22) or may contain multiple kinds of compounds represented by formula (21) and formula (22).

In formula (21) and formula (22), A1, B1, C1, A2, B2, C2, and D2 are preferably each independently selected from a substituted or unsubstituted phenylene group, a substituted or unsubstituted biphenyl group, a substituted or unsubstituted terphenyl group, a substituted or unsubstituted naphthyl group, a substituted or unsubstituted fluorenyl group, a substituted or unsubstituted dibenzofuranyl group, a substituted or unsubstituted dibenzothiophenyl group, and a substituted or unsubstituted carbazolyl group.

It is more preferable that at least one of A1, B1, and C1 in formula (21) and at least one of A2, B2, C2, and D2 in formula (22) be a substituted or unsubstituted biphenyl group, a substituted or unsubstituted terphenyl group, a substituted or unsubstituted naphthyl group, a substituted or unsubstituted fluorenyl group, a substituted or unsubstituted dibenzofuranyl group, or a substituted or unsubstituted dibenzothiophenyl group, a substituted or unsubstituted carbazolyl group.

The fluorenyl group which A1, B1, C1, A2, B2, C2, and D2 may take may have a substituent on the 9-position and may be 9,9-dimethylfluorenyl group or a 9,9-diphenylfluorenyl group, for example. Substituents on the 9-position may form a ring, and the substituents on the 9-position may form a fluorene skeleton or a xanthene skeleton, for example.

L^{A1}, L^{B1}, L^{C1}, L^{A2}, L^{B2}, L^{C2}, and L^{D2} are preferably each independently a single bond or a substituted or unsubstituted arylene group having 6 to 12 ring carbon atoms.

Specific examples of the compounds represented by formula (21) and formula (22) include the following compounds.

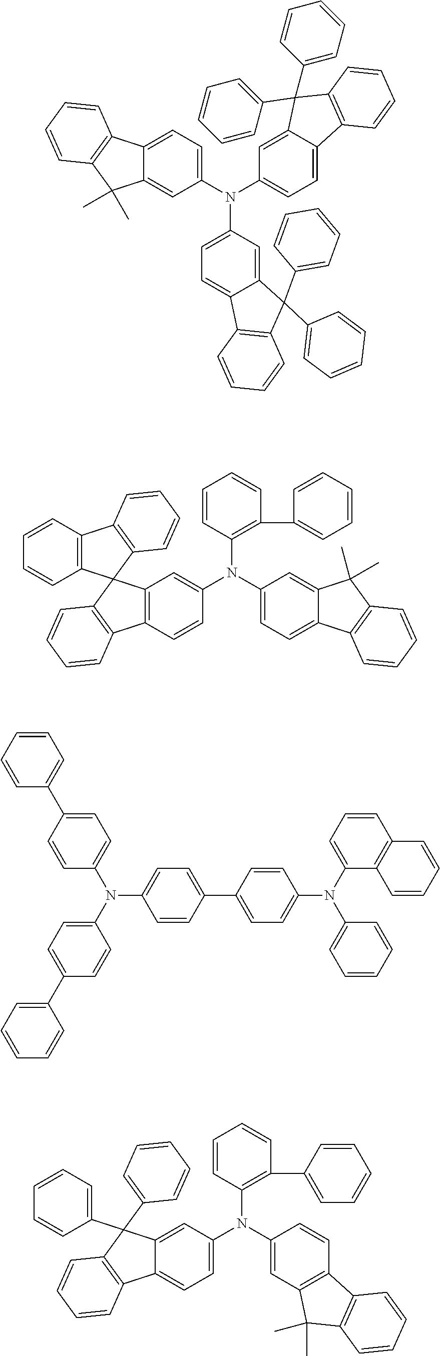 [SMILES: CC1(C)c2ccccc2-c2ccc(N(c3ccc4c(c3)C(c3ccccc3)(c3ccccc3)c3ccccc3-4)c3ccc4c(c3)C(c3ccccc3)(c3ccccc3)c3ccccc3-4)cc21 ; CC1(C)c2ccccc2-c2ccc(N(c3ccc4c(c3)C3(c5ccccc5-c5ccccc53)c3ccccc3-4)c3ccccc3-c3ccccc3)cc21 ; c1ccc(-c2ccc(N(c3ccc(-c4ccccc4)cc3)c3ccc(-c4ccc(N(c5ccccc5)c5cccc6ccccc56)cc4)cc3)cc2)cc1 ; CC1(C)c2ccccc2-c2ccc(N(c3ccc4c(c3)C(c3ccccc3)(c3ccccc3)c3ccccc3-4)c3ccccc3-c3ccccc3)cc21]

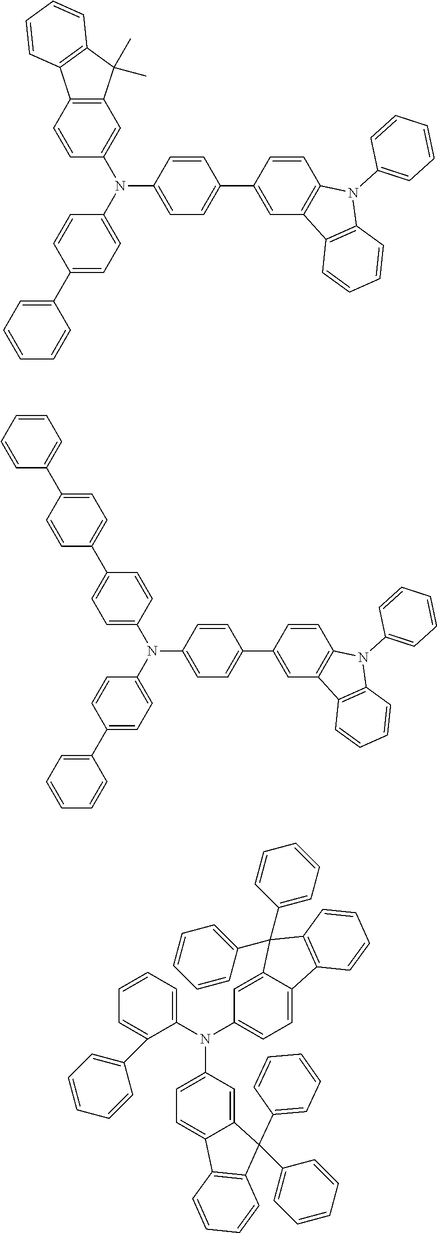 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(-c3ccc(N(c4ccc(-c5ccccc5)cc4)c4ccc(-c5ccc6c(c5)c5ccccc5n6-c5ccccc5)cc4)cc3)cc2)cc1 ; c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1]

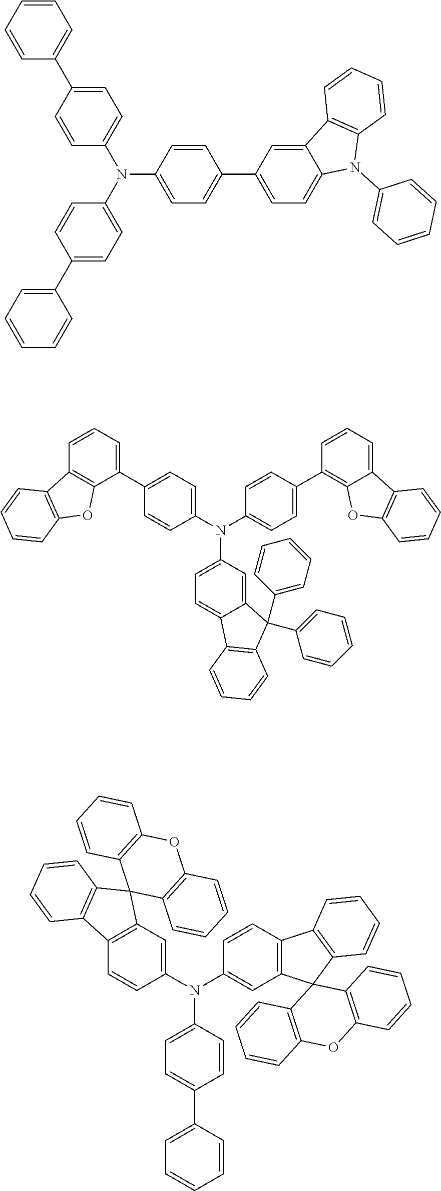 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; c1ccc(C2(c3ccccc3)c3ccccc3-c3ccc(N(c4ccc(-c5cccc6c5oc5ccccc56)cc4)c4ccc(-c5cccc6c5oc5ccccc56)cc4)cc32)cc1 ; c1ccc(-c2ccc(N(c3ccc4c(c3)C3(c5ccccc5Oc5ccccc53)c3ccccc3-4)c3ccc4c(c3)C3(c5ccccc5Oc5ccccc53)c3ccccc3-4)cc2)cc1]

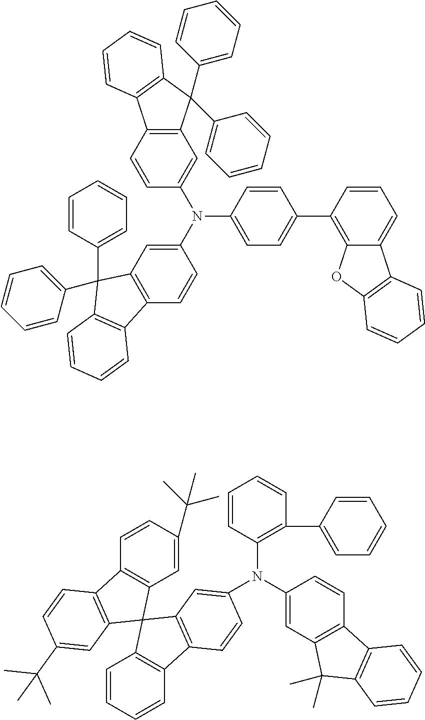 [SMILES: c1ccc(C2(c3ccccc3)c3ccccc3-c3ccc(N(c4ccc(-c5cccc6c5oc5ccccc56)cc4)c4ccc5c(c4)C(c4ccccc4)(c4ccccc4)c4ccccc4-5)cc32)cc1 ; CC(C)(C)c1ccc2c(c1)C1(c3ccccc3-c3ccc(N(c4ccc5c(c4)C(C)(C)c4ccccc4-5)c4ccccc4-c4ccccc4)cc31)c1cc(C(C)(C)C)ccc1-2]

### Dopant Material for Light Emitting Layer

The light emitting layer is a layer including a material (dopant material) having high light emitting performance, and various materials can be used. For example, a fluorescent light emitting material and a phosphorescent light emitting material can be used as the dopant material. The fluorescent light emitting material is a compound that emits light from a singlet excited state, and the phosphorescent light emitting material is a compound that emits light from a triplet excited state.

In one embodiment of the organic EL device according to the present invention, the light emitting layer is a single layer.

In another embodiment of the organic EL device according to the present invention, the light emitting layer includes a first light emitting layer and a second light emitting layer.

As a blue fluorescent light emitting material which can be used for the light emitting layer, a pyrene derivative, a styrylamine derivative, a chrysene derivative, a fluoranthene derivative, a fluorene derivative, a diamine derivative, a triarylamine derivative, or the like can be used. Specific examples include N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), and 4-(10-phenyl-9-anthryl)-4′-(9-phenyl-9H-carbazol-3-yl)triphenylamine (abbreviation: PCBAPA).

As a green fluorescent light emitting material which can be used for the light emitting layer, an aromatic amine derivative or the like can be used. Specific examples include N-(9,10-diphenyl-2-anthryl)-N,9-diphenyl-9H-carbazol-3-amine (abbreviation: 2PCAPA), N-[9,10-bis(1,1′-biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), N-[9,10-bis(1,1′-biphenyl-2-yl)]-N-[4-(9H-carbazol-9-yl)phenyl]-N-phenylanthracen-2-amine (abbreviation: 2YGABPhA), and N,N,9-triphenylanthracene-9-amine (abbreviation: DPhAPhA).

As a red fluorescent light emitting material which can be used for the light emitting layer, a tetracene derivative, a diamine derivative, or the like can be used. Specific examples include N,N,N′,N′-tetrakis(4-methylphenyl)tetracene-5,11-diamine (abbreviation: p-mPhTD) and 7,14-diphenyl-N,N,N′,N′-tetrakis(4-methylphenyl)acenaphtho[1,2-a]fluoranthene-3,10-diamine (abbreviation: p-mPhAFD).

In one embodiment of the present invention, the light emitting layer preferably includes a fluorescent light emitting material (fluorescent dopant material).

As a blue phosphorescent light emitting material which can be used for the light emitting layer, a metal complex such as an iridium complex, an osmium complex, or a platinum complex is used. Specific examples include bis[2-(4′,6′-difluorophenyl)pyridinato-N,C2′]iridium(III)tetrakis(1-pyrazolyl)borate (abbreviation: FIr6), bis[2-(4′,6′-difluorophenyl)pyridinato-N,C2′]iridium(III)picolinate (abbreviation: FIrpic), bis[2-(3′,5′ bistrifluoromethylphenyl)pyridinato-N,C2′]iridium(III)picolinate (abbreviation: Ir(CF3ppy)2(pic)), and bis[2-(4′,6′-difluorophenyl)pyridinato-N,C2′]iridium(III)acetylacetonate (abbreviation: FIracac).

As a green phosphorescent light emitting material which can be used for the light emitting layer, an iridium complex or the like is used. Examples thereof include tris(2-phenylpyridinato-N,C2′)iridium(III) (abbreviation: Ir(ppy)3), bis(2-phenylpyridinato-N,C2′)iridium(III) acetylacetonate (abbreviation: Ir(ppy)2(acac)), bis(1,2-diphenyl-1H-benzimidazolato)iridium(III) acetylacetonate (abbreviation: Ir(pbi)2(acac)), and bis(benzo[h]quinolinato)iridium(III) acetylacetonate (abbreviation: Ir(bzq)2(acac)).

As a red phosphorescent light emitting material which can be used for the light emitting layer, a metal complex such as an iridium complex, a platinum complex, a terbium complex, or a europium complex is used. Specific examples include organic metal complexes such as bis[2-(2′-benzo[4,5-a]thienyl)pyridinato-N,C3′]iridium(III) acetylacetonate (abbreviation: Ir(btp)2(acac)), bis(1-phenylisoquinolinato-N,C2′)iridium(III) acetylacetonate (abbreviation: Ir(piq)2(acac)), (acetylacetonato)bis[2,3-bis(4-fluorophenyl)quinoxalinato]iridium(III) (abbreviation: Ir(Fdpq)2(acac)), and 2,3,7,8,12,13,17,18-octaethyl-21H,23H-porphyrinplatinum(II) (abbreviation: PtOEP).

Rare earth metal complexes such as tris(acetylacetonato)(monophenanthroline)terbium(III) (abbreviation: Tb(acac)3(Phen)), tris(1,3-diphenyl-1,3-propanedionato)(monophenanthroline)europium(III) (abbreviation: Eu(DBM)3(Phen)), and tris[1-(2-thenoyl)-3,3,3-trifluoroacetonato](monophenanthroline)europium(III) (abbreviation: Eu(TTA)3(Phen)) can be used as a phosphorescent light emitting material since such rare earth metal complexes emit light from rare earth metal ions (electron transition between different multiplicities).

### Host Material for Light Emitting Layer

The light emitting layer may have a configuration in which the above-described dopant material is dispersed in another material (a host material). A material which has the lowest unoccupied molecular orbital level (LUMO level) higher than that of the dopant material and has the highest occupied molecular orbital level (HOMO level) lower than that of the dopant material is preferably used.

As the host material,
(1) a metal complex such as an aluminum complex, a beryllium complex, or a zinc complex,
(2) a heterocyclic compound such as an oxadiazole derivative, a benzimidazole derivative, or a phenanthroline derivative,
(3) a condensed aromatic compound such as a carbazole derivative, an anthracene derivative, a phenanthrene derivative, a pyrene derivative, or a chrysene derivative, or
(4) an aromatic amine compound such as a triarylamine derivative or a condensed polycyclic aromatic amine derivative is used.

For example, metal complexes such as tris(8-quinolinolato)aluminum(III) (abbreviation: Alq), tris(4-methyl-8-quinolinolato)aluminum(III) (abbreviation: Almq3), bis(10-hydroxybenzo[h]quinolinato)beryllium(II) (abbreviation: BeBq2), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), and bis[2-(2-benzothiazolyl)phenolato]zinc(II) (abbreviation: ZnBTZ);
heterocyclic compounds such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzimidazole) (abbreviation: TPBI), bathophenanthroline (abbreviation: BPhen), and bathocuproine (abbreviation: BCP);
condensed aromatic compounds such as 9-[4-(10-phenyl-9-anthryl)phenyl]-9H-carbazole (abbreviation: CzPA), 3,6-diphenyl-9-[4-(10-phenyl-9-anthryl)phenyl]-9H-carbazole (abbreviation: DPCzPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 3,3′,3″-(benzene-1,3,5-triyl)tripyrene (abbreviation: TPB3), 9,10-diphenylanthracene (abbreviation: DPAnth), and 6,12-dimethoxy-5,11-diphenylchrysene; and
aromatic amine compounds such as N,N-diphenyl-9-[4-(10-phenyl-9-anthryl)phenyl]-9H-carbazol-3-amine (abbreviation: CzA1PA), 4-(10-phenyl-9-anthryl)triphenylamine (abbreviation: DPhPA), N,9-diphenyl-N-[4-(10-phenyl-9-anthryl)phenyl]-9H-carbazol-3-amine (abbreviation: PCAPA), N,9-diphenyl-N-{4-[4-(10-phenyl-9-anthryl)phenyl]phenyl}-9H-carbazol-3-amine (abbreviation: PCAPBA), N-(9,10-diphenyl-2-anthryl)-N,9-diphenyl-9H-carbazol-3-amine (abbreviation: 2PCAPA), 4,4′-bis[N-(1-naphthyl)-N-phenylamino]biphenyl (abbreviation: NPB or α-NPD), N,N′-bis(3-methylphenyl)-N,N′-diphenyl-[1,1′-biphenyl]-4,4′-diamine (abbreviation: TPD), 4,4′-bis[N-(9,9-dimethylfluoren-2-yl)-N-phenylamino]biphenyl (abbreviation: DFLDPBi), and 4,4′-bis[N-(spiro-9,9′-bifluoren-2-yl)-N-phenylamino]biphenyl (abbreviation: BSPB) can be used. Plural kinds of host materials may be used.

In particular, in the case of a blue fluorescent device, the following anthracene compounds are preferably used as the host material.

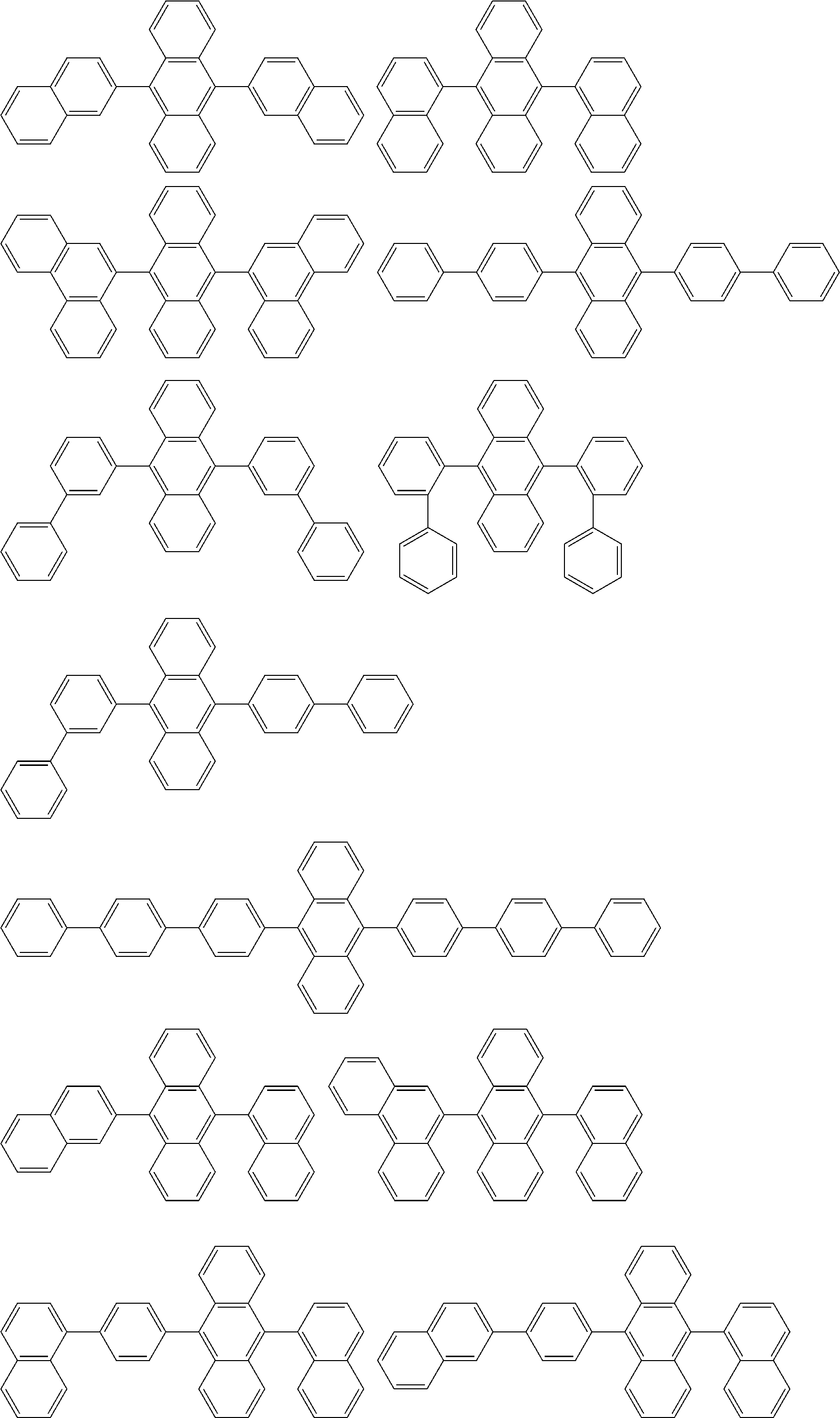

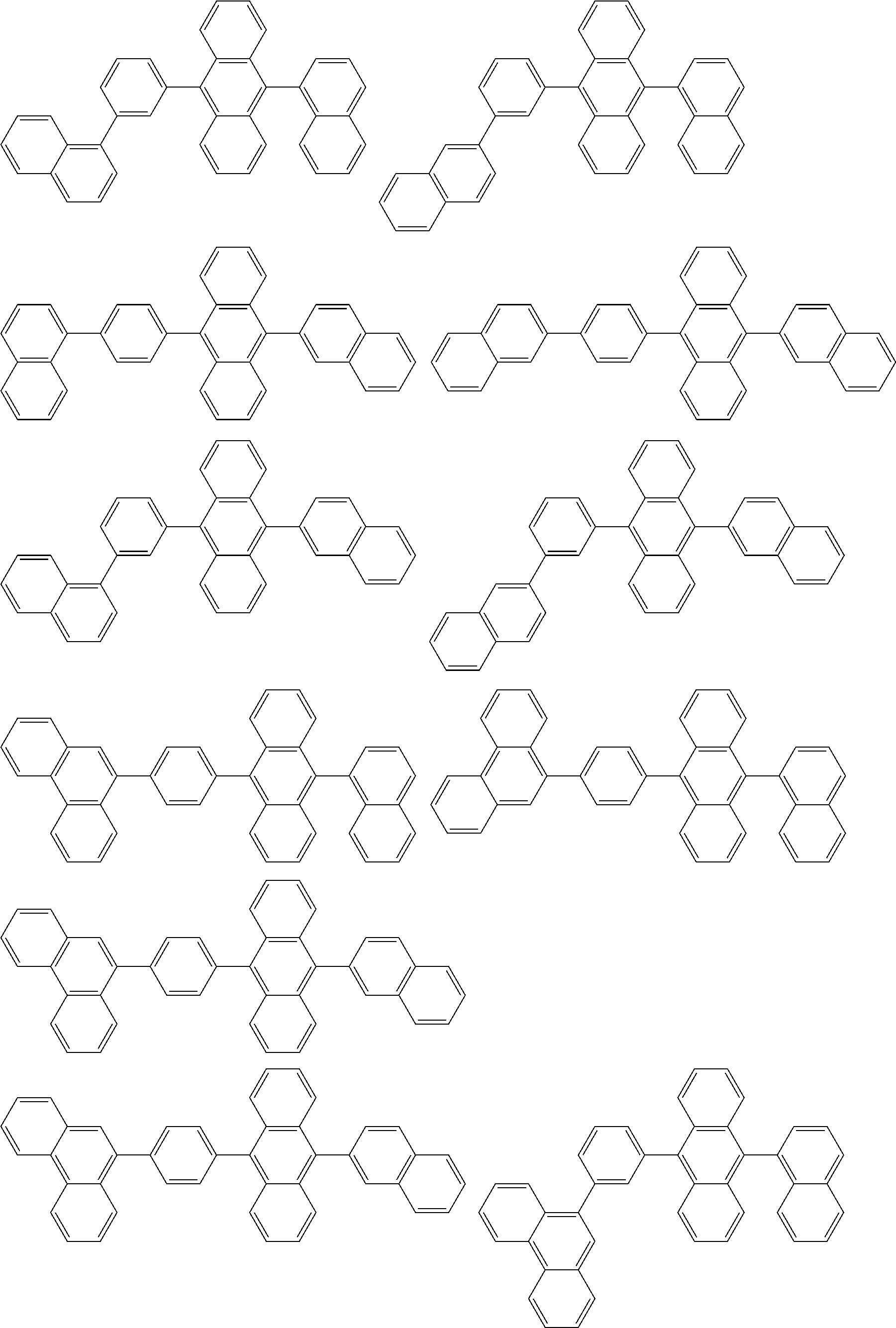

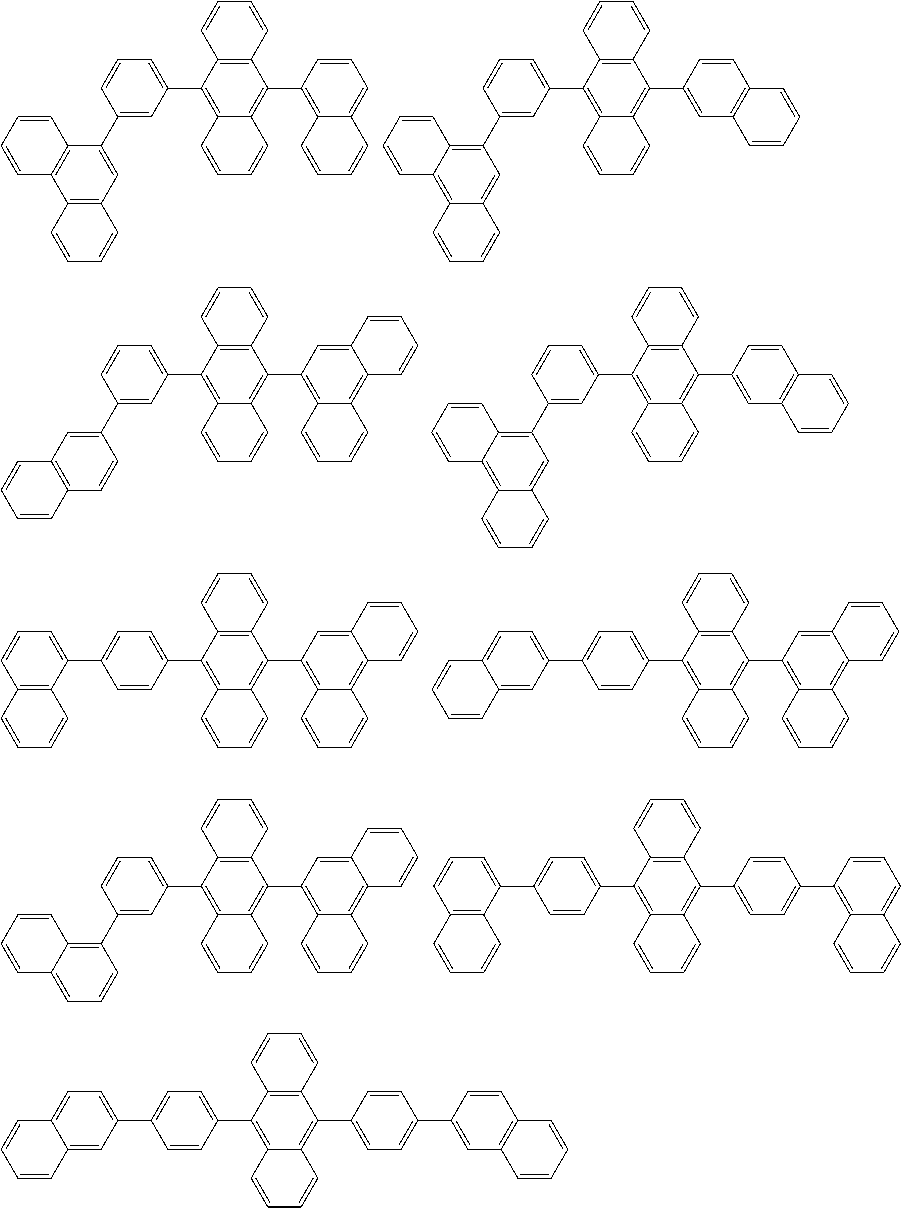

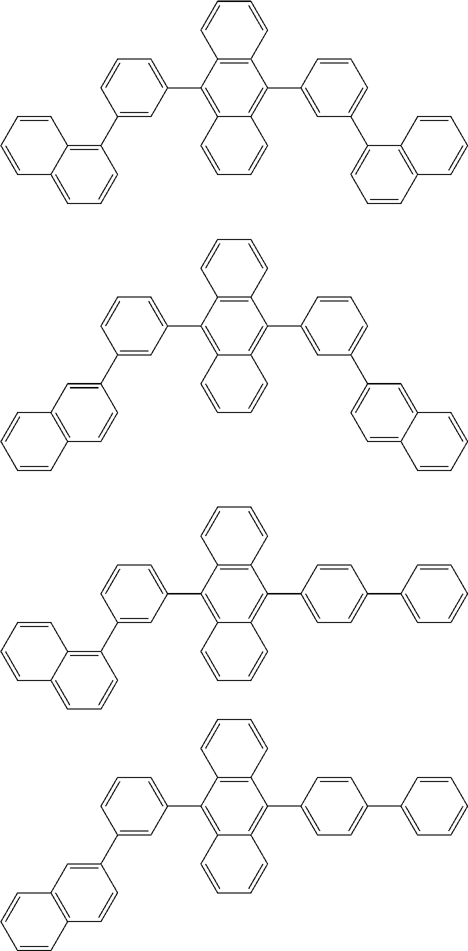

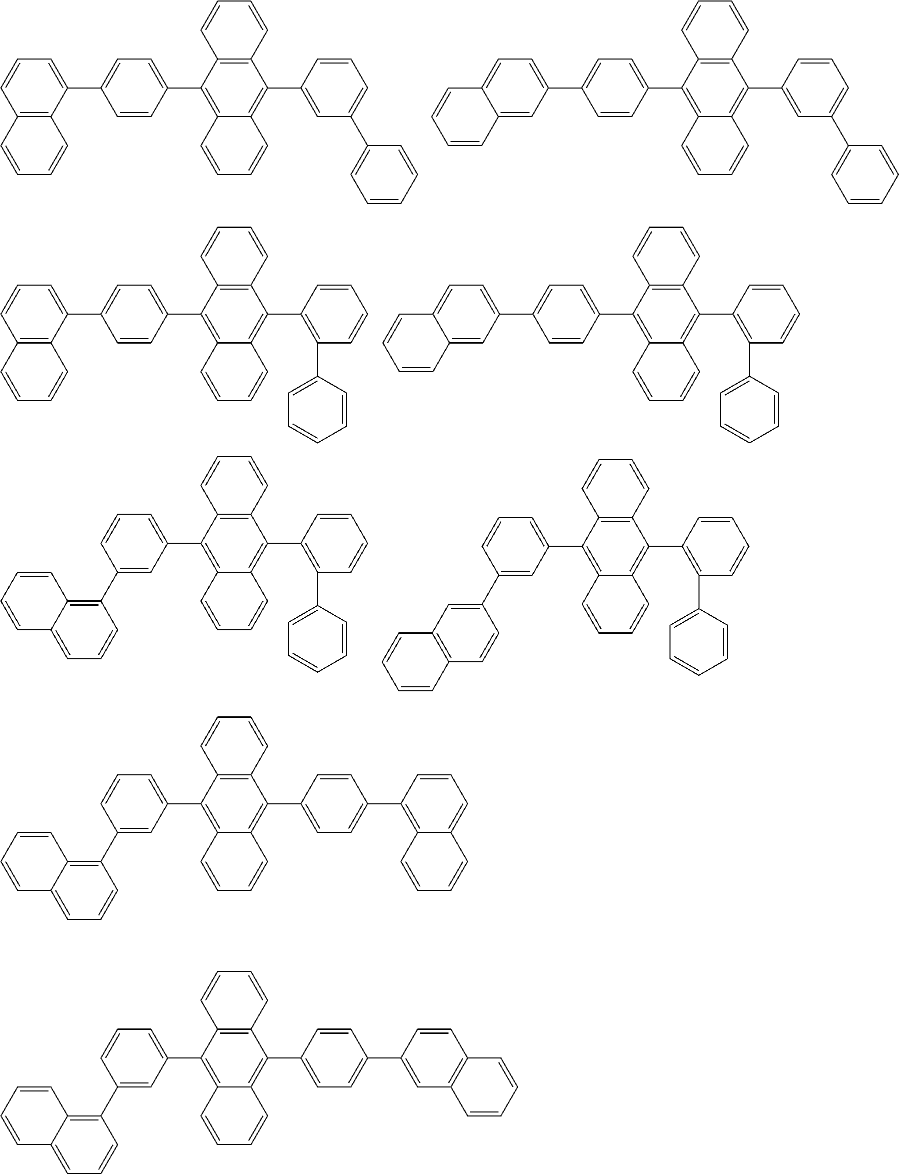

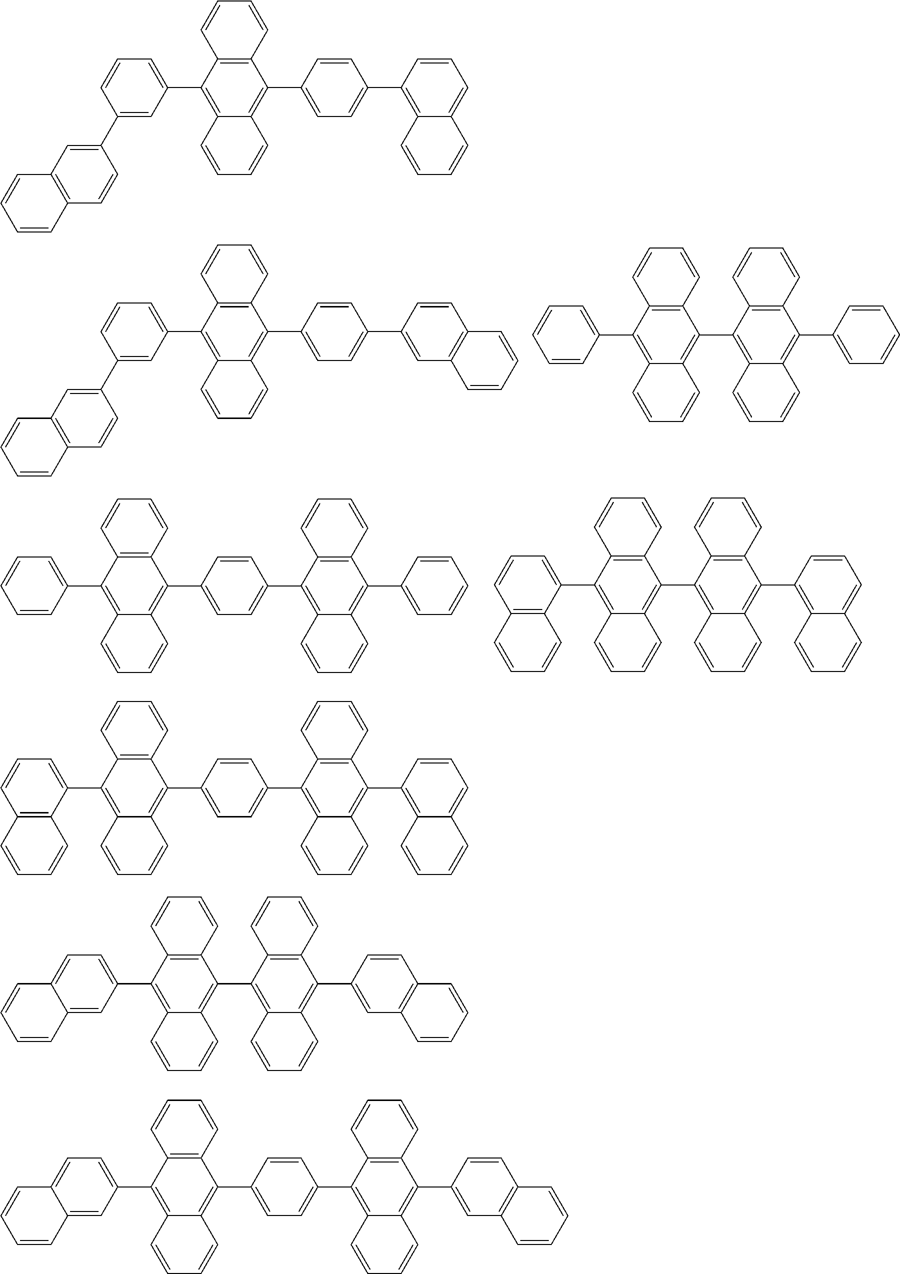

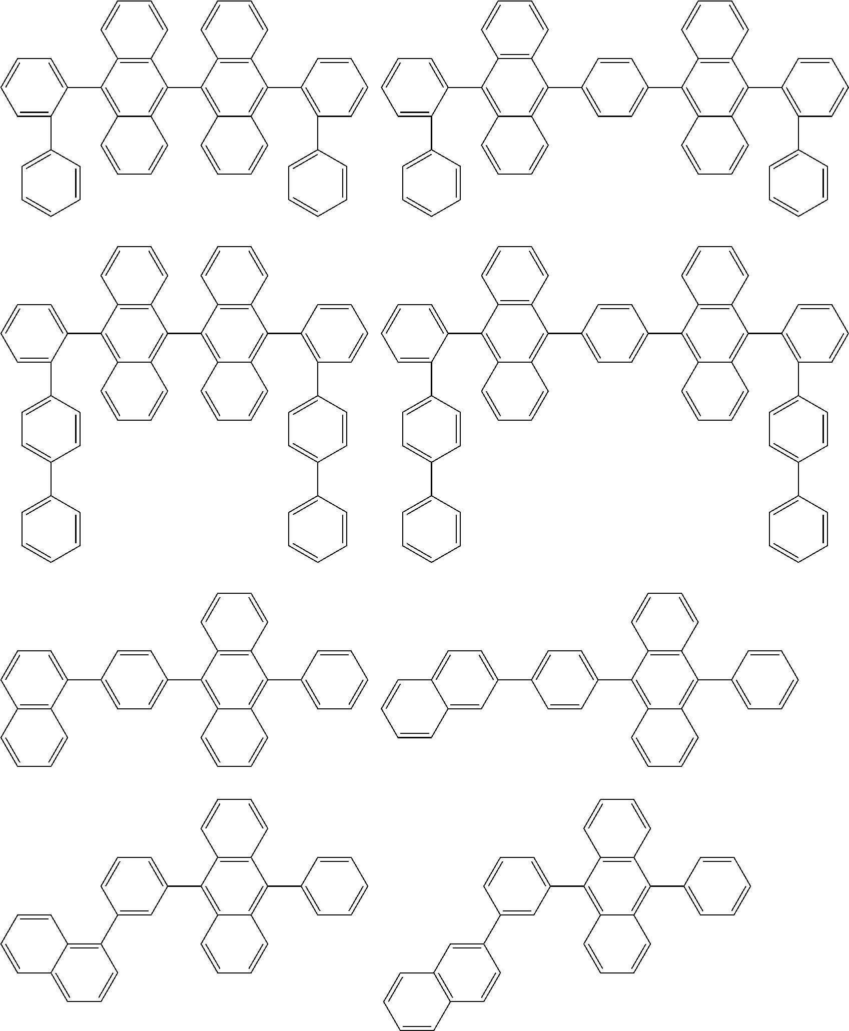

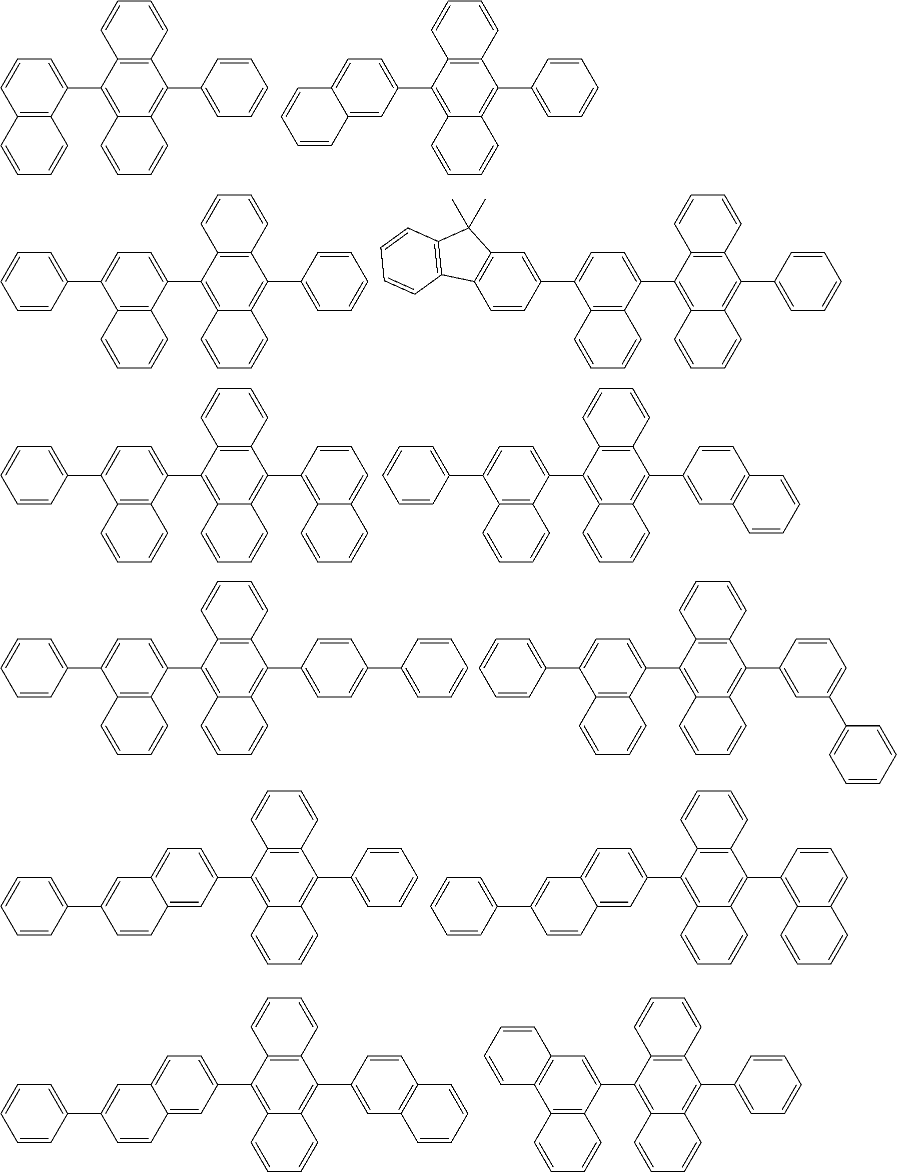

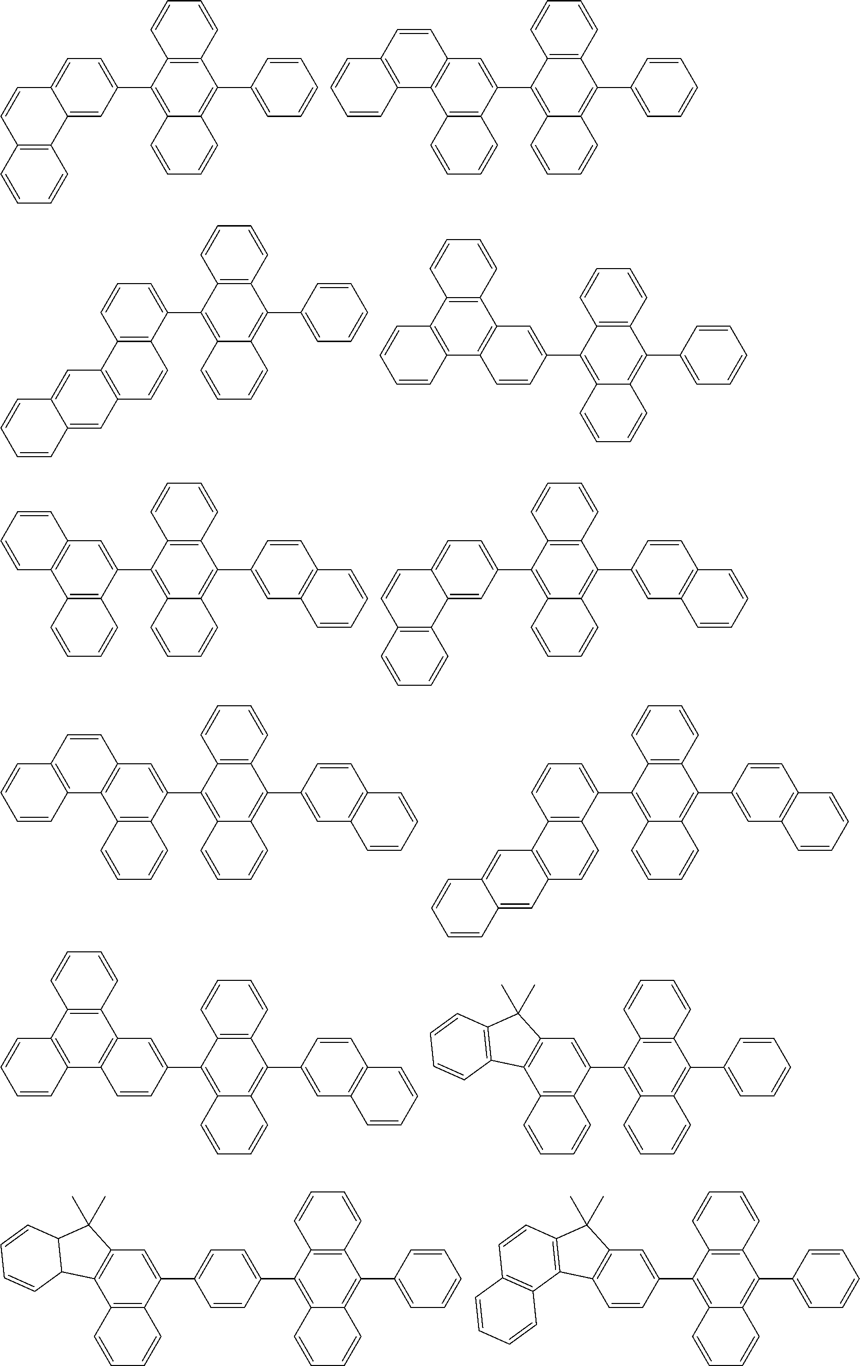

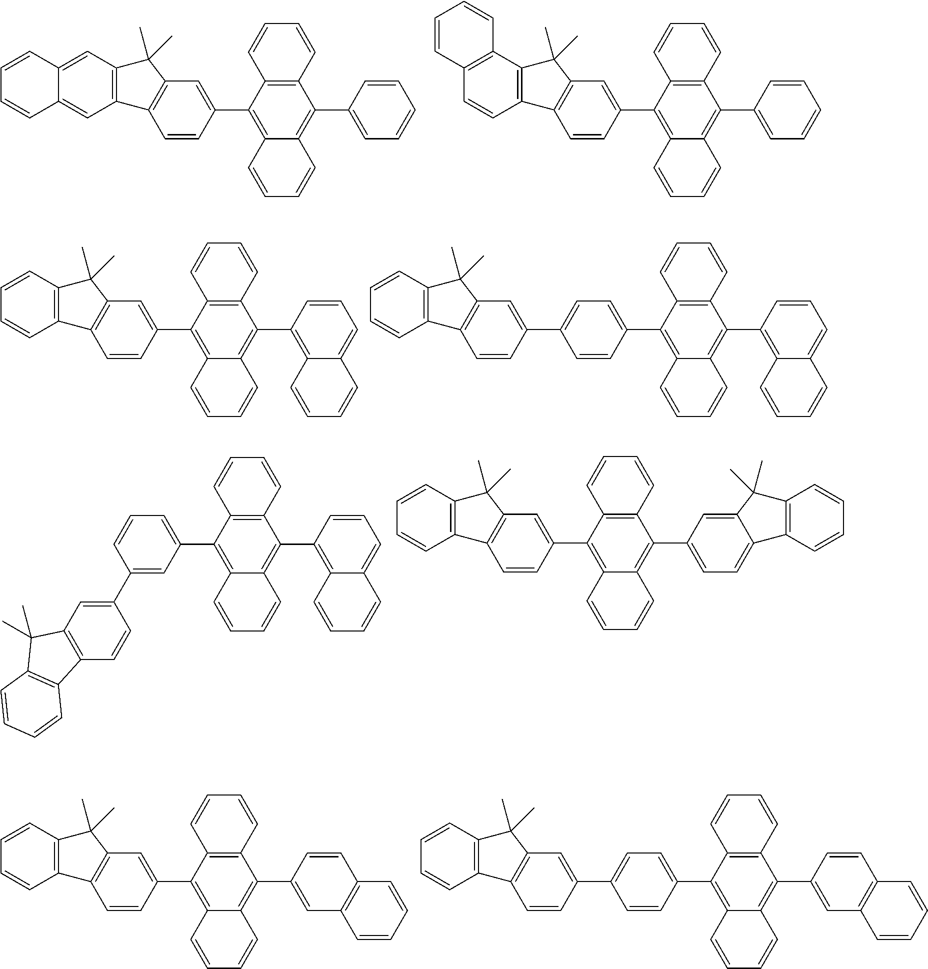

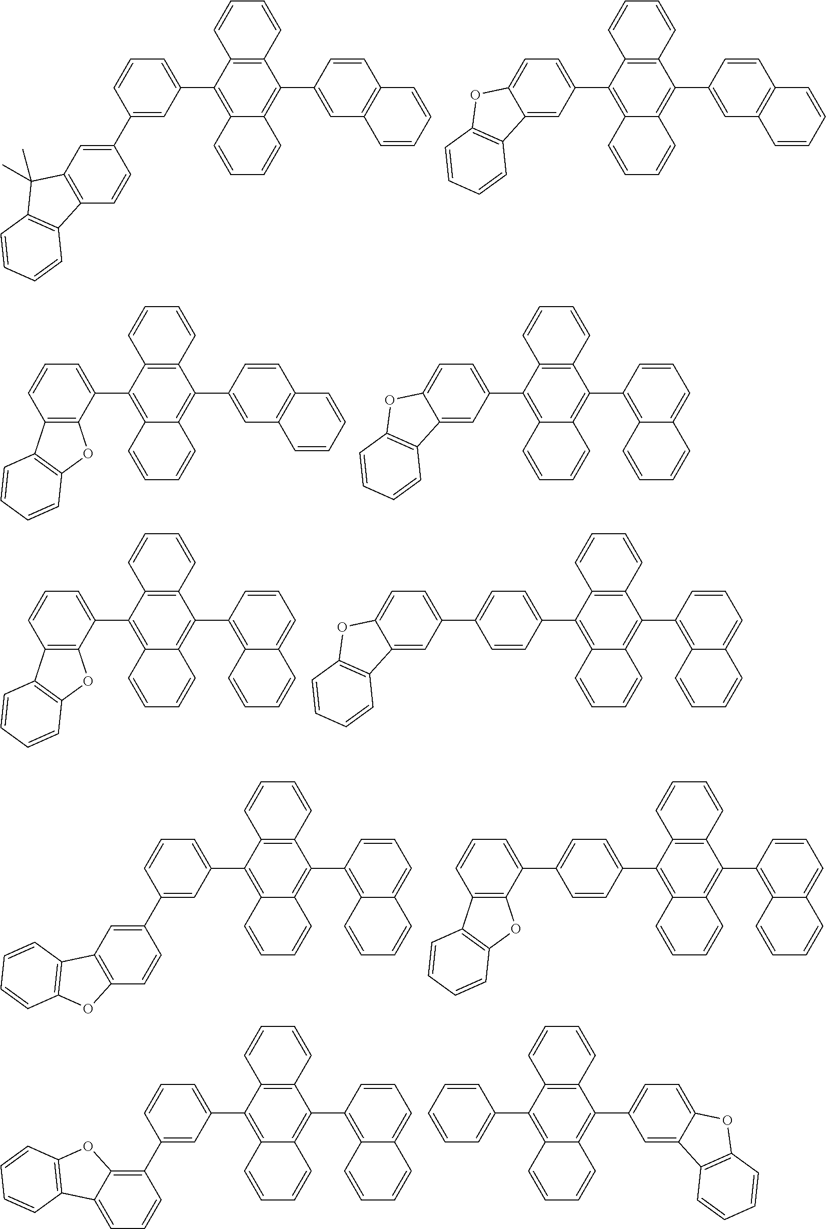

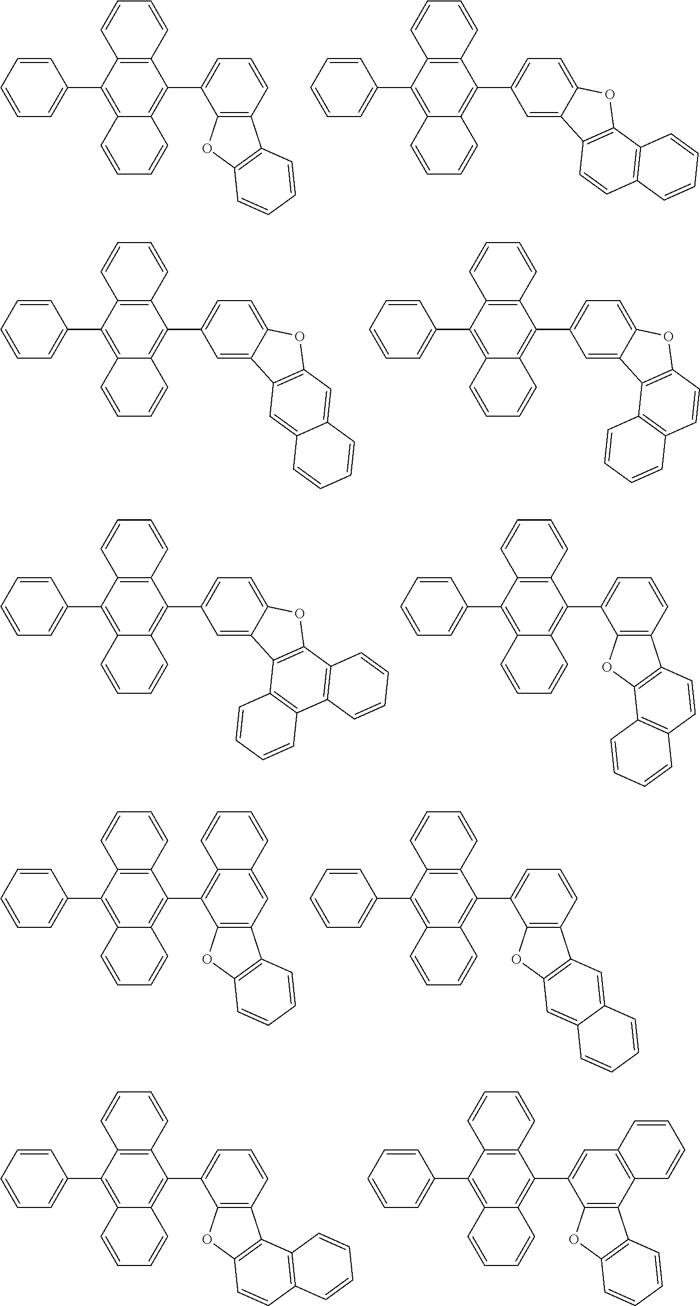

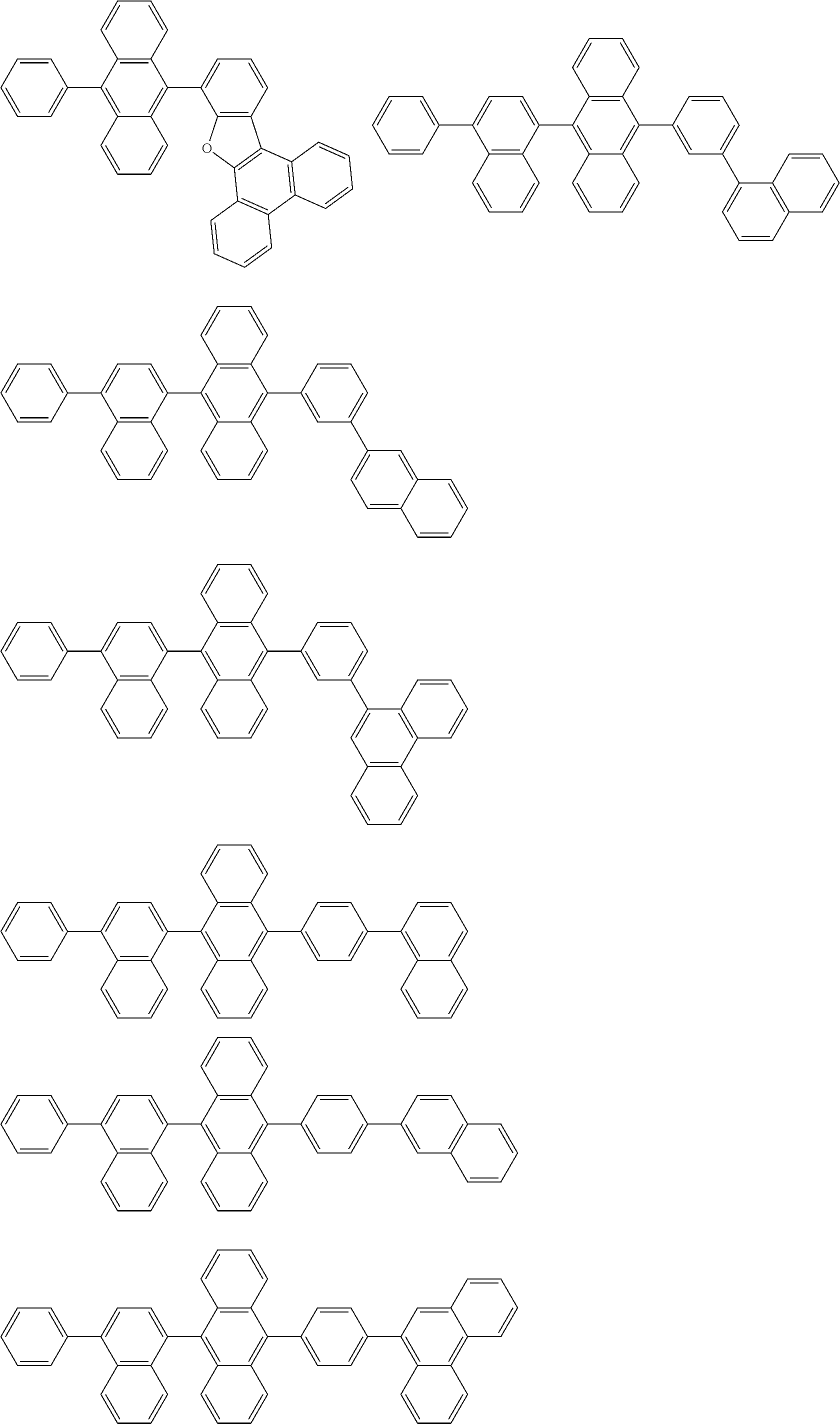

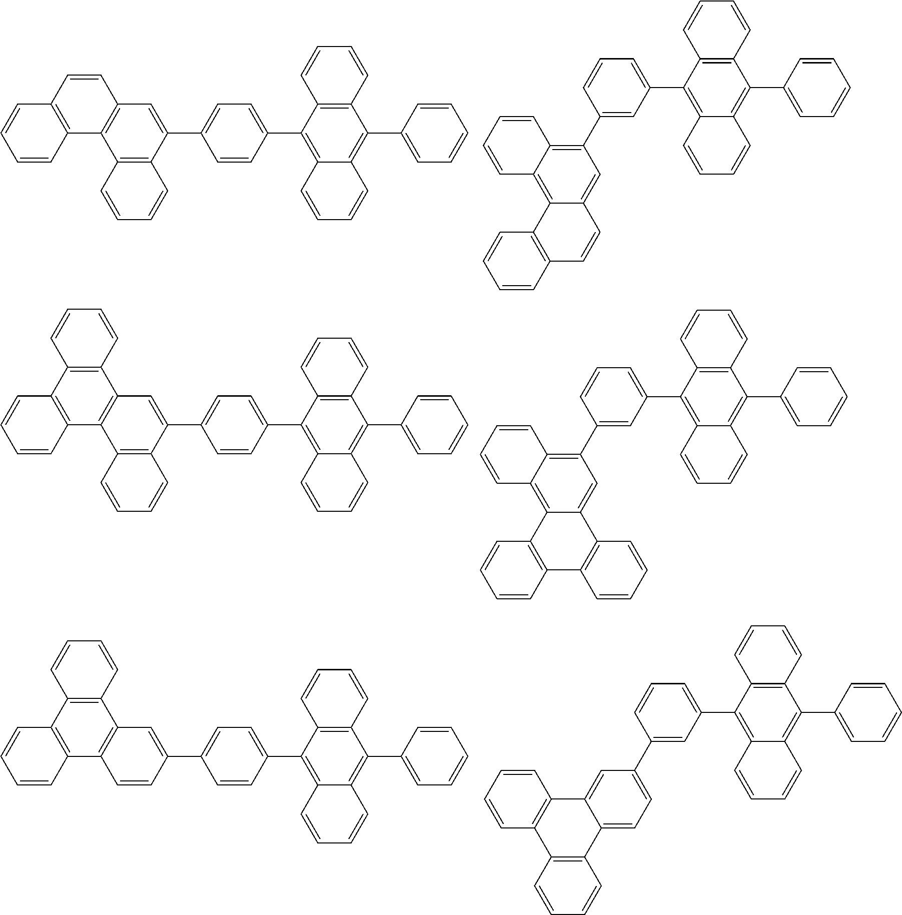

In one embodiment of the organic EL device according to the present invention, when the light emitting layer includes the first light emitting layer and the second light emitting layer, at least one component constituting the first light emitting layer is different from a component constituting the second light emitting layer. For example, an embodiment in which the dopant material included in the first light emitting layer is different from the dopant material included in the second light emitting layer, and an embodiment in which the host material included in the first light emitting layer is different from the host material included in the second light emitting layer are exemplified.

In the organic EL device according to the present embodiment, the light emitting layer may contain a light emitting compound that emits fluorescent light having a main peak wavelength of 500 nm or less.

The main peak wavelength of the compound is measured by the following method. A 5 μmol/L toluene solution of the compound to be measured is prepared and placed in a quartz cell, and the emission spectrum (vertical axis: emission intensity, horizontal axis: wavelength) of the sample is measured at room temperature (300 K). The emission spectrum can be measured with a fluorescence spectrophotometer (device name: F-7000) manufactured by Hitachi High-Tech Science Corporation. The emission spectrum measurement apparatus is not limited to the apparatus used here.

In the emission spectrum, an emission spectrum peak wavelength at which the emission intensity is maximized is defined as the main peak wavelength. In this specification, the main peak wavelength is sometimes referred to as a fluorescence emission main peak wavelength (FL-peak).

The light emitting compound emitting fluorescent light having a main peak wavelength of 500 nm or less may be the dopant material or the host material described above.

When the light emitting layer is a single layer, only one of the dopant material and the host material may be the light emitting compound emitting fluorescent light having a main peak wavelength of 500 nm or less, or both of the dopant material and the host material may be the light emitting compound emitting fluorescent light having a main peak wavelength of 500 nm or less.

In the case where the light emitting layer includes the first light emitting layer and the second light emitting layer, only one of the first light emitting layer and the second light emitting layer may include the light emitting compound emitting fluorescent light having a main peak wavelength of 500 nm or less, or both of the light emitting layers may include the light emitting compound emitting fluorescent light having a main peak wavelength of 500 nm or less. In the case where the first light emitting layer includes the light emitting compound emitting fluorescent light having a main peak wavelength of 500 nm or less, only one of the dopant material and the host material included in the first light emitting layer may be the light emitting compound emitting fluorescent light having a main peak wavelength of 500 nm or less, or both of the materials may be the light emitting compound emitting fluorescent light having a main peak wavelength of 500 nm or less. In the case where the second light emitting layer includes the light emitting compound emitting fluorescent light having a main peak wavelength of 500 nm or less, only one of the dopant material and the host material included in the second light emitting layer may be the light emitting compound emitting fluorescent light having a main peak wavelength of 500 nm or less, or both of the materials may be the light emitting compound emitting fluorescent light having a main peak wavelength of 500 nm or less.

### Electron Transporting Layer

The electron transporting layer is a layer including a material having high electron transporting performance (electron transporting material), and is formed between the light emitting layer and the cathode or between the electron injecting layer and the light emitting layer when present.

The electron transporting layer may have a single layer structure or a multilayer structure including two or more layers. For example, the electron transporting layer may have a two-layer structure including a first electron transporting layer (on the anode side) and a second electron transporting layer (on the cathode side). In one embodiment of the present invention, it is preferable that the electron transporting layer of the single layer structure be adjacent to the light emitting layer, and it is preferable that an electron transporting layer closest to the anode in the multilayer structure, for example, the first electron transporting layer of the two-layer structure be adjacent to the light emitting layer. In another embodiment of the present invention, a hole blocking layer, which will be described later, may be interposed between the electron transporting layer of the single layer structure and the light emitting layer or between the light emitting layer and the electron transporting layer closest to the light emitting layer in the multilayer structure.

For the electron transporting layer,
(1) a metal complex such as an aluminum complex, a beryllium complex, or a zinc complex,
(2) a heteroaromatic compound such as an imidazole derivative, a benzimidazole derivative, an azine derivative, a carbazole derivative, or a phenanthroline derivative, or
(3) a high molecular weight compound can be used, for example.

Examples of the metal complex include tris(8-quinolinolato)aluminum(III) (abbreviation: Alq), tris(4-methyl-8-quinolinolato)aluminum (abbreviation: Almq3), bis(10-hydroxybenzo[h]quinolinato)beryllium (abbreviation: BeBq₂), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), and bis[2-(2-benzothiazolyl)phenolato]zinc(II) (abbreviation: ZnBTZ).

Examples of the heteroaromatic compound include 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazol-2-yl)stilbene (abbreviation: BzOs).

Examples of the high molecular weight compound include poly[(9,9-dihexylfluorene-2,7-diyl)-co-(pyridine-3,5-diyl)](abbreviation: PF-Py) and poly[(9,9-dioctylfluorene-2,7-diyl)-co-(2,2′-bipyridine-6,6′-diyl)](abbreviation: PF-BPy).

The above-described materials have electron mobility of 10⁻⁶ cm²/Vs or more. Note that a material other than the materials mentioned above may be used for the electron transporting layer as long as it exhibits higher electron transporting performance compared with hole transporting performance.

### Electron Injecting Layer

The electron-injecting layer is a layer including a material having high electron injecting performance. For the electron injecting layer, an alkali metal such as lithium (Li) or cesium (Cs), an alkaline earth metal such as magnesium (Mg), calcium (Ca), or strontium (Sr), a rare-earth metal such as europium (Eu) or ytterbium (Yb), and a compound including any of these metals can be used. Examples of such compounds include alkali metal oxides, alkali metal halides, alkali metal-containing organic complexes, alkaline earth metal oxides, alkaline earth metal halides, alkaline earth metal-containing organic complexes, rare earth metal oxides, rare earth metal halides, and rare earth metal-containing organic complexes. A mixture of a plurality of these compounds may also be used.

Besides, an electron transporting material containing an alkali metal, an alkaline earth metal, or a compound thereof, specifically, Alq containing magnesium (Mg) or the like may be used. In this case, electron injection from the cathode can be performed more efficiently.

Alternatively, a composite material in which an organic compound and an electron donor (donor) are mixed may be used for the electron injecting layer. Such a composite material is excellent in electron injecting performance and electron transporting performance because the organic compound receives electrons from the electron donor. In this case, the organic compound is preferably a material excellent in transporting the received electrons. Specifically, any of the above-described materials constituting the electron transporting layer (a metal complex, a heteroaromatic compound, etc.) can be used. As the electron donor, a material exhibiting ability to donate electrons to the organic compound may be used. Specifically, an alkali metal, an alkaline earth metal, and a rare earth metal are preferable, and examples thereof include lithium, cesium, magnesium, calcium, erbium, and ytterbium. In addition, an alkali metal oxide and an alkaline earth metal oxide are preferable, and examples thereof include a lithium oxide, a calcium oxide, and a barium oxide. A Lewis base such as magnesium oxide can also be used. An organic compound such as tetrathiafulvalene (abbreviation: TTF) can also be used.

### Cathode

As the cathode, a metal, an alloy, an electrically conductive compound, and a mixture thereof having a low work function (specifically, a work function of 3.8 eV or less) are preferably used. Specific examples of such a cathode material include elements belonging to Group 1 or 2 of the periodic table of the elements, that is, alkali metals such as lithium (Li) and cesium (Cs), alkaline earth metals such as magnesium (Mg), calcium (Ca), and strontium (Sr), and alloys containing these elements (for example, MgAg and AlLi), rare earth metals such as europium (Eu) and ytterbium (Yb), and alloys containing these elements.

Note that when the cathode is formed using an alkali metal, an alkaline earth metal, or an alloy thereof, a vacuum deposition method or a sputtering method can be used. In a case of using silver paste or the like, a coating method or an inkjet method can be used.

When the electron injecting layer is provided, the cathode can be formed using any of various conductive materials such as Al, Ag, ITO, graphene, and indium oxide-tin oxide containing silicon or silicon oxide regardless of the work function. These conductive materials can be formed into a film using a sputtering method, an inkjet method, or a spin coating method.

### Insulating Layer

Since an electric field is applied to an ultrathin film in the organic EL device, pixel defects due to leakage or short circuit are likely to generate. In order to prevent generation of pixel defects, an insulating layer made of an insulating thin film layer may be inserted between a pair of electrodes.

Examples of the material used for the insulating layer include aluminum oxide, lithium fluoride, lithium oxide, cesium fluoride, cesium oxide, magnesium oxide, magnesium fluoride, calcium oxide, calcium fluoride, aluminum nitride, titanium oxide, silicon oxide, germanium oxide, silicon nitride, boron nitride, molybdenum oxide, ruthenium oxide, and vanadium oxide. A mixture or a laminate of these materials may be used.

### Space Layer

The space layer is, for example, a layer provided between the fluorescent light emitting layer and the phosphorescent light emitting layer for the purpose of preventing excitons generated in the phosphorescent light emitting layer from diffusing into the fluorescent light emitting layer or for the purpose of adjusting carrier balance, when the fluorescent light emitting layer and the phosphorescent light emitting layer are laminated. The space layer can be provided between multiple phosphorescent light emitting layers.

Since the space layer is provided between the light emitting layers, the material therefore preferably has both electron transporting performance and hole transporting performance. In order to prevent diffusion of triplet energy in an adjacent phosphorescent light emitting layer, the triplet energy is preferably 2.6 eV or more. Examples of the material used for the space layer include the same materials as those used for the hole transporting layer described above.

### Blocking Layer

A blocking layer such as an electron blocking layer, a hole blocking layer, or an exciton blocking layer may be provided adjacent to the light emitting layer. The electron blocking layer is a layer that prevents electrons from leaking from the light emitting layer to the hole transporting layer, and the hole blocking layer is a layer that prevents holes from leaking from the light emitting layer to the electron transporting layer. The exciton blocking layer has a function to prevent excitons generated in the light emitting layer from diffusing into adjacent layers and to confine excitons within the light-emitting layer.

Each layer of the organic EL device can be formed by a conventionally known vapor deposition method or a coating method. For example, each layer can be formed by a known method including: a deposition method such as a vacuum deposition method and a molecular beam epitaxy method (MBE method); and a coating method using a solution of a compound for forming a layer, such as a dipping method, a spin coating method, a casting method, a bar coating method, and a roll coating method.

Although the film thickness of each layer is not particularly limited, in general, when the film thickness is too thin, defects such as pinholes are likely to generate; on the contrary, when the film thickness is too thick, a high driving voltage is required, and efficiency is deteriorated. Therefore, the film thickness is usually 5 nm to 10 m, and is more preferably 10 nm to 0.2 m.

In one embodiment of the organic EL device of the present invention, the total thickness of the thicknesses of the first hole transporting layer and the thickness of the second hole transporting layer is 30 nm or more and 150 nm or less. In this case, the thickness is preferably 40 nm or more and 130 nm or less.

In one embodiment of the organic EL device of the present invention, the thickness of the second hole transporting layer is 20 nm or more. The thickness is preferably 25 nm or more and more preferably 35 nm or more, and is preferably 100 nm or less.

In one embodiment of the organic EL device of the present invention, the hole transporting layer adjacent to the light emitting layer is 20 nm or more. The thickness is preferably 25 nm or more and more preferably 30 nm or more, and is preferably 100 nm or less.

In one embodiment of the organic EL device of the present invention, the film thickness D1 of the first hole transporting layer and the film thickness D2 of the second hole transporting layer satisfy the relationship of 0.3<D2/D1<4.0. The D1 and D2 preferably satisfy the relationship of 0.5<D2/D1<3.5, and more preferably satisfy the relationship of 0.75<D2/D1<3.0.

### EXEMPLARY EMBODIMENTS OF THE ORGANIC EL DEVICE OF THE PRESENT INVENTION INCLUDE

an organic EL device having the hole transporting layer having the two-layer structure, in which the second hole transporting layer includes the compound of the present invention, and the first hole transporting layer does not include the compound of the present invention (first embodiment);
an organic EL device having the hole transporting layer having the two-layer structure, in which the first hole transporting layer and the second hole transporting layer both include the compound of the present invention (second embodiment);
an organic EL device having the hole transporting layer having the two-layer structure, in which the first hole transporting layer includes the compound of the present invention, and the second hole transporting layer does not include the compound of the present invention (third embodiment);
an organic EL device having the hole transporting layer having the three-layer structure, in which the first hole transporting layer includes the compound of the present invention, and the second and third hole transporting layers do not include the compound of the present invention (fourth embodiment);
an organic EL device having the hole transporting layer having the three-layer structure, in which the second hole transporting layer includes the compound of the present invention, and the first and third hole transporting layers do not include the compound of the present invention (fifth embodiment);
an organic EL device having the hole transporting layer having the three-layer structure, in which the third hole transporting layer includes the compound of the present invention, and the first and second hole transporting layers do not include the compound of the present invention (sixth embodiment);
an organic EL device having the hole transporting layer having the three-layer structure, in which the first and second hole transporting layers include the compound of the present invention, and the third hole transporting layer does not include the compound of the present invention (seventh embodiment);
an organic EL device having the hole transporting layer having the three-layer structure, in which the first and third hole transporting layers include the compound of the present invention, and the second hole transporting layer does not include the compound of the present invention (eighth embodiment);
an organic EL device having the hole transporting layer having the three-layer structure, in which the second and third hole transporting layers include the compound of the present invention, and the first hole transporting layer does not include the compound of the present invention (tenth embodiment); and
an organic EL device having the hole transporting layer having the three-layer structure, in which the first to third hole transporting layers all include the compound of the present invention (tenth embodiment).

### Electronic Device

The organic EL device can be used in electronic devices such as a display component including an organic EL panel module, a display device including a television, a mobile phone, and a personal computer, and a light emitting device of a lighting device or a vehicle lamp.

### EXAMPLES

Hereinafter, the present invention will be described in more detail with reference to Examples, but the present invention is not limited thereto.

### Compounds Used to Produce Organic EL Devices of Examples 1-3

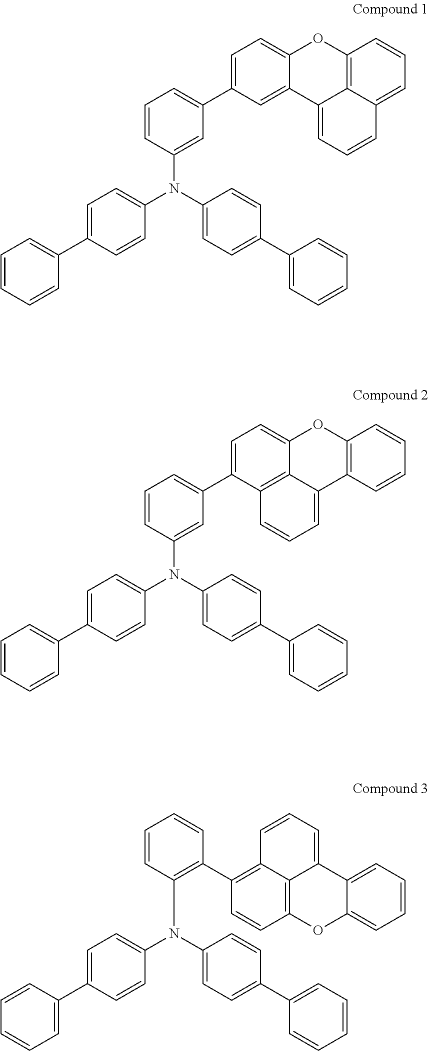 [SMILES: c1ccc(-c2ccc(N(c3ccc(-c4ccccc4)cc3)c3cccc(-c4ccc5c(c4)-c4cccc6cccc(c46)O5)c3)cc2)cc1 ; c1ccc(-c2ccc(N(c3ccc(-c4ccccc4)cc3)c3cccc(-c4ccc5c6c(cccc46)-c4ccccc4O5)c3)cc2)cc1 ; c1ccc(-c2ccc(N(c3ccc(-c4ccccc4)cc3)c3ccccc3-c3ccc4c5c(cccc35)-c3ccccc3O4)cc2)cc1]

### Compound Used to Produce Organic EL Device of Comparative Example 1

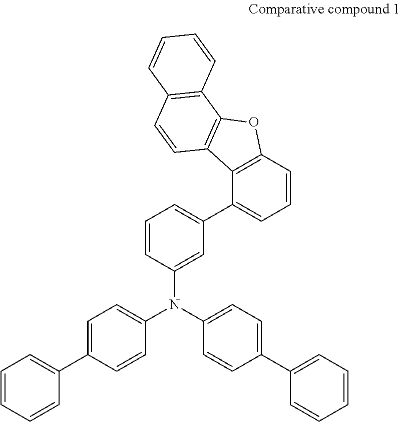 [SMILES: c1ccc(-c2ccc(N(c3ccc(-c4ccccc4)cc3)c3cccc(-c4cccc5oc6c7ccccc7ccc6c45)c3)cc2)cc1]

### Other Compounds Used to Produce Organic EL Devices of Examples 1-3 and Comparative Example 1

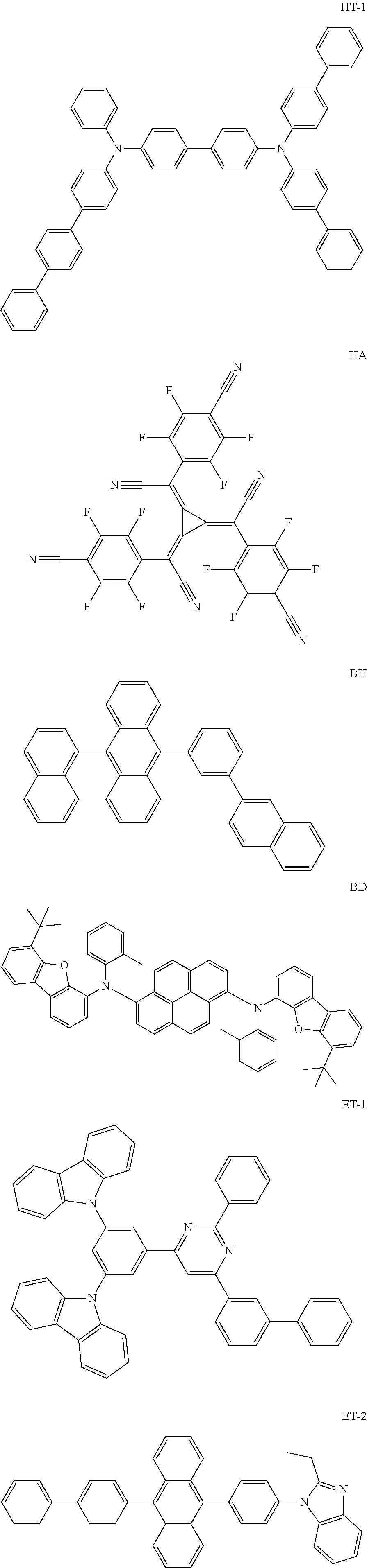

### Production of Organic EL Devices

### Example 1

A glass substrate with an ITO transparent electrode (anode) of 25 mm×75 mm×1.1 mm (manufactured by GEOMATEC Co., Ltd.) was subjected to ultrasonic cleaning in isopropyl alcohol for 5 minutes and then to UV ozone cleaning for 30 minutes. The film thickness of the ITO was 130 nm.

The glass substrate with the transparent electrode after cleaning was mounted on a substrate holder of a vacuum deposition device, and compound HT-1 and compound HA were co-deposited onto the surface on the side where the transparent electrode was formed so as to cover the transparent electrode, thereby forming a hole injecting layer having a film thickness of 10 nm. The mass ratio (HT-1:HA) between compound HT-1 and compound HA was 97:3.

Then, compound HT-1 was vapor-deposited on the hole injecting layer to form a first hole transporting layer having a film thickness of 80 nm.

Then, compound 1 was vapor-deposited as compound HT-2 on the first hole transporting layer to form a second hole transporting layer having a film thickness of 10 nm.

Then, compound BH (host material) and compound BD (dopant material) were co-deposited on the second hole transporting layer to form a light emitting layer having a film thickness of 25 nm. The mass ratio (BH:BD) between compound BH and compound BD was 96:4.

Then, compound ET-1 was vapor-deposited on the light-emitting layer to form a first electron transporting layer having a film thickness of 10 nm.

Then, compound ET-2 was vapor-deposited on the first electron transporting layer to form a second electron transporting layer having a film thickness of 15 nm.

Then, LiF was vapor-deposited on the second electron transporting layer to form an electron injecting electrode having a film thickness of 1 nm.

Then, Al metal was vapor-deposited on the electron injecting electrode to form a metal cathode having a film thickness of 50 nm.

The layer structure of the organic EL device of Example 1 thus obtained is shown below.

$$ITO ( 1 \; 30 ) / HT - 1 : HA = 97 : 3 ( 10 ) / HT - 1 ( 80 ) / HT - 2 ( 10 ) / BH : BD = 96 : 4 ( 25 ) / ET - 1 ( 10 ) / ET - 2 ( 15 ) / Li F ( 1 ) / A 1 ( 50 )$$

In the above layer structure, each number in the parentheses represents the film thickness (nm), and the ratios are in terms of mass.

### Examples 2 and 3

Organic EL devices were produced in the same manner as in Example 1 except that the material for the second hole transporting layer was changed from compound 1 to compound 2 and compound 3, respectively, as shown in Table 1 below.

### Comparative Example 1

An organic EL device was produced in the same manner as in Example 1 except that the material for the second hole transporting layer was changed from compound 1 to comparative compound 1.

### Evaluation of Organic EL Device

A voltage was applied to the organic EL device produced in each of Examples 1-3 and Comparative Example 1 so that the current density was 10 mA/cm², and the voltage value at that time was measured and taken as the driving voltage.

Further, a voltage was applied to each of the organic EL devices so that the current density was 50 mA/cm², and the 95% lifetime (LT95) was evaluated. Here, the 95% lifetime (LT95) refers to a time (hr) until the luminance drops to 95% of the initial luminance during constant current driving.

Results are shown in Table 1.

TABLE 1

| | Second hole | | |
|---|---|---|---|
| | transporting | | |
| | material | Driving voltage [V] | LT95 [h] |
| | (HT-2) | @10 mA/cm² | @50 mA/cm² |
| Example 1 | Compound 1 | 3.86 | 170 |
| Example 2 | Compound 2 | 3.85 | 190 |
| Example 3 | Compound 3 | 3.82 | 177 |
| Comparative | Comparative | 3.92 | 133 |
| Example 1 | compound 1 | | |

As is clear from the results of Table 1, the compounds (compounds 1 to 3 of Examples 1 to 3) satisfying the requirements of the present invention are found to exhibit significantly improved driving voltage and LT95 values compared to the monoamine (comparative compound 1 of Comparative Example 1) not satisfying the requirements of the present invention.

### Compound Used to Produce Organic EL Device of Example 4

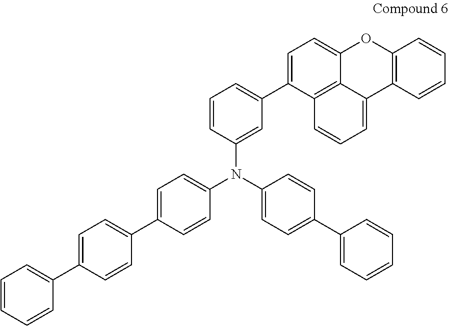 [SMILES: c1ccc(-c2ccc(-c3ccc(N(c4ccc(-c5ccccc5)cc4)c4cccc(-c5ccc6c7c(cccc57)-c5ccccc5O6)c4)cc3)cc2)cc1]

### Comparative Compound Used to Produce Organic EL Device of Comparative Example 2

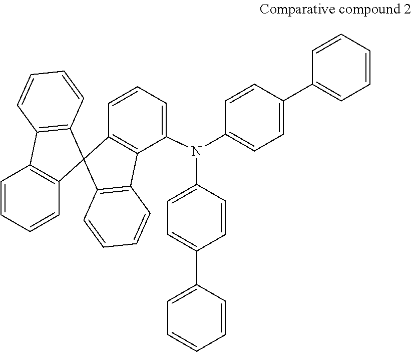 [SMILES: c1ccc(-c2ccc(N(c3ccc(-c4ccccc4)cc3)c3cccc4c3-c3ccccc3C43c4ccccc4-c4ccccc43)cc2)cc1]

### Other Compounds Used to Produce Organic EL Devices of Example 4 and Comparative Example 2

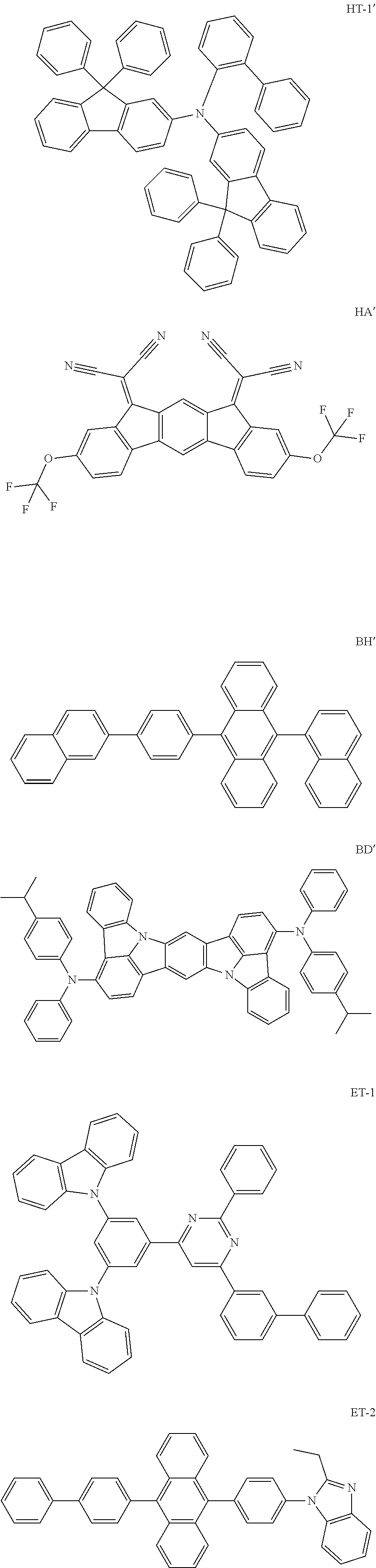

### Production of Organic EL Devices

### Example 4

A glass substrate with an ITO transparent electrode (anode) of 25 mm×75 mm×1.1 mm (manufactured by GEOMATEC Co., Ltd.) was subjected to ultrasonic cleaning in isopropyl alcohol for 5 minutes and then to UV ozone cleaning for 30 minutes. The film thickness of the ITO was 130 nm.

The glass substrate with the transparent electrode after cleaning was mounted on a substrate holder of a vacuum deposition device, and compound HA′ was vapor-deposited onto the surface on the side where the transparent electrode was formed so as to cover the transparent electrode, thereby forming a hole injecting layer having a film thickness of 5 nm.

Then, compound HT-1′ was vapor-deposited on the hole injecting layer to form a first hole transporting layer having a film thickness of 80 nm.

Then, compound 6 was vapor-deposited as compound HT-2 on the first hole transporting layer to form a second hole transporting layer having a film thickness of 10 nm.

Then, compound BH′ (host material) and compound BD′ (dopant material) were co-deposited on the second hole transporting layer to form a light emitting layer having a film thickness of 25 nm. The mass ratio (BH′:BD′) between compound BH′ and compound BD′ was 98:2.

Then, compound ET-1 was vapor-deposited on the light-emitting layer to form a first electron transporting layer having a film thickness of 10 nm.

Then, compound ET-2 was vapor-deposited on the first electron transporting layer to form a second electron transporting layer having a film thickness of 15 nm.

Then, LiF was vapor-deposited on the second electron transporting layer to form an electron injecting electrode having a film thickness of 1 nm.

Then, Al metal was vapor-deposited on the electron injecting electrode to form a metal cathode having a film thickness of 80 nm.

The layer structure of the organic EL device of Example 4 thus obtained is shown below.

$$ITO ( 1 \; 30 ) / {HA}^{′} ( 5 ) / HT - {1}^{′} ( 80 ) / HT - 2 ( 10 ) / {BH}^{′} : {BD}^{′} = 98 : 2 ( 25 ) / ET - 1 ( 10 ) / ET - 2 ( 15 ) / Li F ( 1 ) / A 1 ( 80 )$$

In the above layer structure, each number in the parentheses represents the film thickness (nm), and the ratios are in terms of mass.

### Comparative Example 2

An organic EL device was produced in the same manner as in Example 4 except that the material for the second hole transporting layer was changed from compound 6 to comparative compound 2.

### Evaluation of Organic EL Device

A voltage was applied to the organic EL device produced in each of Example 4 and Comparative Example 2 so that the current density was 10 mA/cm², and the voltage value at that time was measured and taken as the driving voltage.

Further, a voltage was applied to each of the organic EL devices so that the current density was 50 mA/cm², and the 95% lifetime (LT95) was evaluated. Here, the 95% lifetime (LT95) refers to a time (hr) until the luminance drops to 95% of the initial luminance during constant current driving.

Results are shown in Table 2.

TABLE 2

| | Second hole | | |
|---|---|---|---|
| | transporting | | |
| | material | Driving voltage [V] | LT95 [h] |
| | (HT-2) | @10 mA/cm² | @50 mA/cm² |
| Example 4 | Compound 6 | 3.76 | 218 |
| Comparative | Comparative | 3.90 | 166 |
| Example 2 | compound 2 | | |

As is clear from the results of Table 2, the compound (compound 6 of Example 4) satisfying the requirements of the present invention is found to exhibit significantly improved driving voltage and LT95 value compared to the monoamine (comparative compound 2 of Comparative Example 2) not satisfying the requirements of the present invention.

### Compounds Synthesized in Synthesis Examples

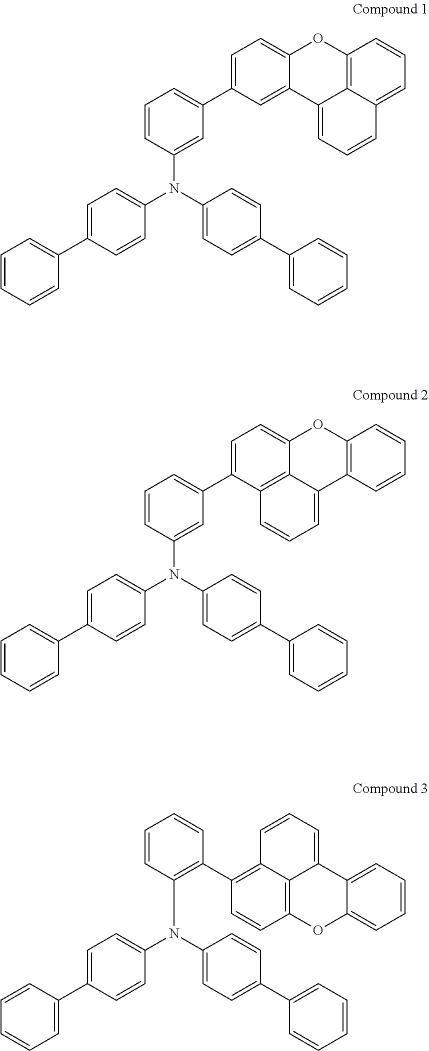 [SMILES: c1ccc(-c2ccc(N(c3ccc(-c4ccccc4)cc3)c3cccc(-c4ccc5c(c4)-c4cccc6cccc(c46)O5)c3)cc2)cc1 ; c1ccc(-c2ccc(N(c3ccc(-c4ccccc4)cc3)c3cccc(-c4ccc5c6c(cccc46)-c4ccccc4O5)c3)cc2)cc1 ; c1ccc(-c2ccc(N(c3ccc(-c4ccccc4)cc3)c3ccccc3-c3ccc4c5c(cccc35)-c3ccccc3O4)cc2)cc1]

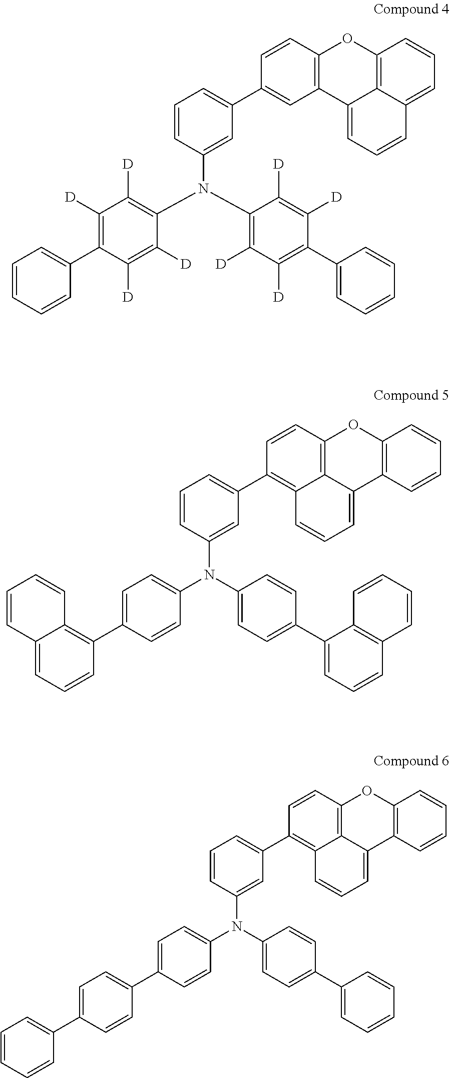 [SMILES: [2H]c1c([2H])c(N(c2cccc(-c3ccc4c(c3)-c3cccc5cccc(c35)O4)c2)c2c([2H])c([2H])c(-c3ccccc3)c([2H])c2[2H])c([2H])c([2H])c1-c1ccccc1 ; c1cc(-c2ccc3c4c(cccc24)-c2ccccc2O3)cc(N(c2ccc(-c3cccc4ccccc34)cc2)c2ccc(-c3cccc4ccccc34)cc2)c1 ; c1ccc(-c2ccc(-c3ccc(N(c4ccc(-c5ccccc5)cc4)c4cccc(-c5ccc6c7c(cccc57)-c5ccccc5O6)c4)cc3)cc2)cc1]

### Synthesis of Compounds

### Intermediate Synthesis Example 1: Synthesis of Intermediate A

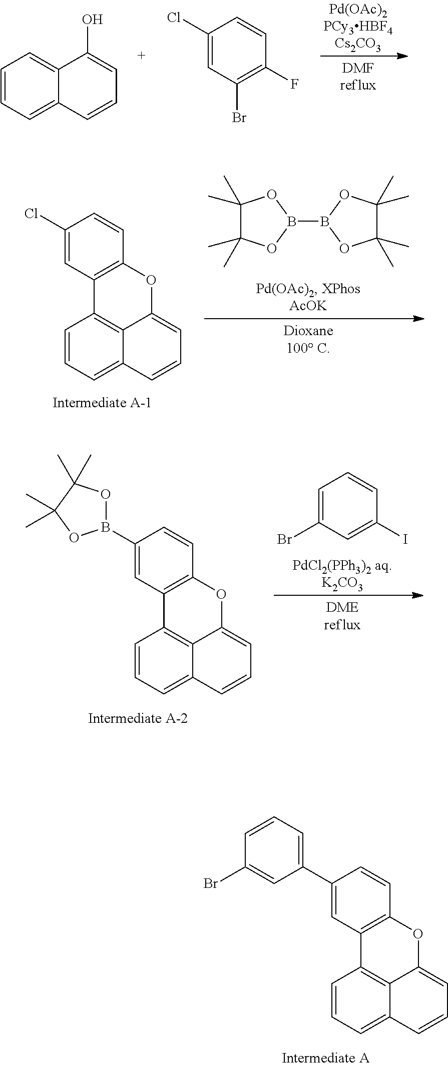

### Synthesis of Intermediate A-1

A mixture of naphthalene-1-ol (9.37 g, 64.99 mmol), 2-bromo-4-chloro-1-fluorobenzene (16.34 g, 78.01 mmol), palladium(II) acetate (1.46 g, 6.50 mmol), tricyclohexylphosphonium tetrafluoroborate (4.79 g, 13.00 mmol), cesium carbonate (63.50 g, 194.89 mmol), and N,N-dimethylformamide (325 mL) was heated, refluxed, and stirred for 7 hours in an argon atmosphere. The reaction liquid was cooled to room temperature, extracted with ethyl acetate, and subsequently concentrated under reduced pressure. The obtained residue was purified by silica gel column chromatography to obtain intermediate A-1 as a pale-green solid (11.3 g). The yield was 69%.

### Synthesis of Intermediate A-2

A mixture of intermediate A-1 (2.35 g, 9.30 mmol), palladium(II) acetate (0.10 g, 0.445 mmol), 2-dicyclohexylphosphino-2′,4′,6′-triisopropylbiphenyl (0.44 g, 0.922 mmol), bis(pinacolato)diboron (3.54 g, 13.94 mmol), potassium acetate (2.74 g, 27.91 mmol), and 1,4-dioxane (50 mL) was stirred at 100° C. for 4 hours in an argon atmosphere. The reaction liquid was cooled to room temperature, extracted with dichloromethane, and subsequently concentrated under reduced pressure. The obtained residue was purified by a silica gel short column to obtain intermediate A-2 as a white solid (1.98 g). The yield was 62%.

### Synthesis of Intermediate A

A mixture of intermediate A-2 (10.33 g, 30.00 mmol), 1-bromo-3-iodobenzene (8.49 g, 30.00 mmol), dichlorobis(triphenylphosphine)palladium(II) (0.632 g, 0.900 mmol), potassium carbonate (12.44 g, 90.00 mmol), DME (150 mL), and water (45 mL) was stirred at 80° C. for 2 hours in an argon atmosphere. The reaction liquid was cooled to room temperature and concentrated under reduced pressure. The obtained residue was purified by silica gel column chromatography to obtain intermediate A as a white solid (9.85 g). The yield was 88%.

### Intermediate Synthesis Example 2: Synthesis of Intermediate B

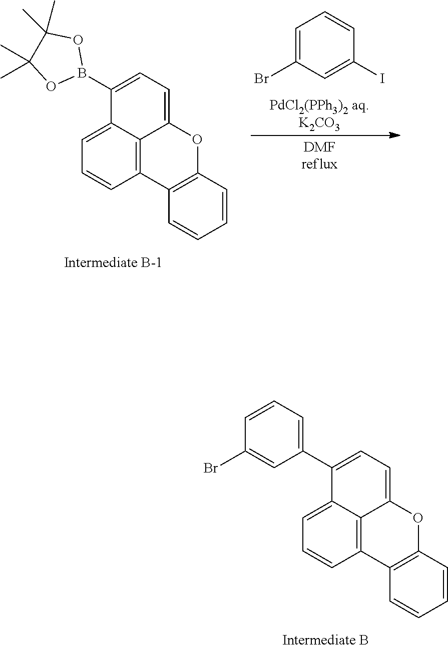 [SMILES: CC1(C)OB(c2ccc3c4c(cccc24)-c2ccccc2O3)OC1(C)C ; Brc1cccc(I)c1 ; Brc1cccc(-c2ccc3c4c(cccc24)-c2ccccc2O3)c1]

Intermediate B was obtained as a white solid in the same manner as in Intermediate Synthesis Example 1 except that a known intermediate B-1 was used instead of intermediate A-2.

### Intermediate Synthesis Example 3: Synthesis of Intermediate C

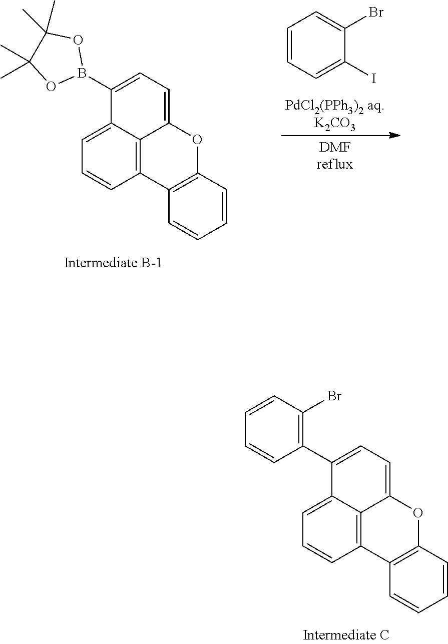 [SMILES: CC1(C)OB(c2ccc3c4c(cccc24)-c2ccccc2O3)OC1(C)C ; Brc1ccccc1I ; Brc1ccccc1-c1ccc2c3c(cccc13)-c1ccccc1O2]

Intermediate C was obtained as a white solid in the same manner as in Intermediate Synthesis Example 2 except that 1-bromo-2-iodobenzene was used instead of 1-bromo-3-iodobenzene.

### Synthesis Example 1: Synthesis of Compound 1

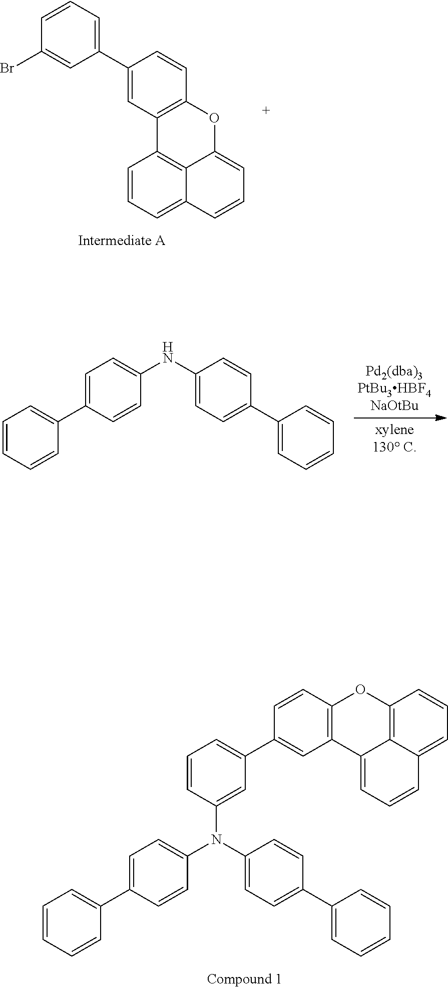 [SMILES: Brc1cccc(-c2ccc3c(c2)-c2cccc4cccc(c24)O3)c1 ; c1ccc(-c2ccc(Nc3ccc(-c4ccccc4)cc3)cc2)cc1 ; c1ccc(-c2ccc(N(c3ccc(-c4ccccc4)cc3)c3cccc(-c4ccc5c(c4)-c4cccc6cccc(c46)O5)c3)cc2)cc1]

A mixture of intermediate A (3.73 g, 10.00 mmol), N-[1,1′-biphenyl]-4-yl-[1,1′-biphenyl]-4-amine (3.21 g, 10.00 mmol), tris(dibenzylideneacetone)dipalladium(0) (0.183 g, 0.200 mmol), tri-t-butylphosphonium tetrafluoroborate (0.232 g, 0.800 mmol), sodium-t-butoxide (1.44 g, 15.00 mmol), and xylenes (50 mL) was stirred at 130° C. for 3 hours in an argon atmosphere. The reaction liquid was cooled to room temperature, and subsequently concentrated under reduced pressure. The obtained residue was purified by silica gel column chromatography and recrystallization to obtain 4.26 g of a white solid. The yield was 69%.

As a result of mass spectrum analysis, the obtained compound was compound 1, in which m/e=614 with respect to the molecular weight of 613.76.

### Synthesis Example 2: Synthesis of Compound 2

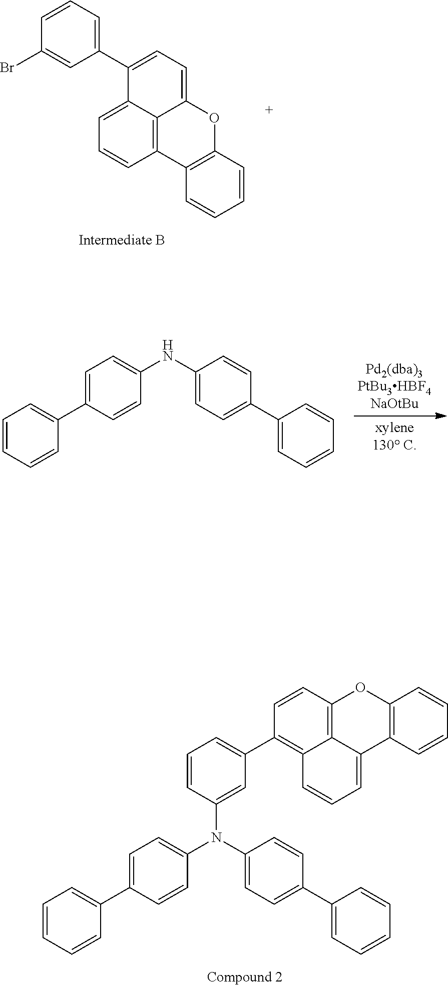 [SMILES: Brc1cccc(-c2ccc3c4c(cccc24)-c2ccccc2O3)c1 ; c1ccc(-c2ccc(Nc3ccc(-c4ccccc4)cc3)cc2)cc1 ; c1ccc(-c2ccc(N(c3ccc(-c4ccccc4)cc3)c3cccc(-c4ccc5c6c(cccc46)-c4ccccc4O5)c3)cc2)cc1]

Compound 2 was obtained in the same manner as in Synthesis Example 1 except that intermediate B was used instead of intermediate A used in Synthesis Example 1.

As a result of mass spectrum analysis, the obtained compound was compound 2, in which m/e=614 with respect to the molecular weight of 613.76.

### Synthesis Example 3: Synthesis of Compound 3

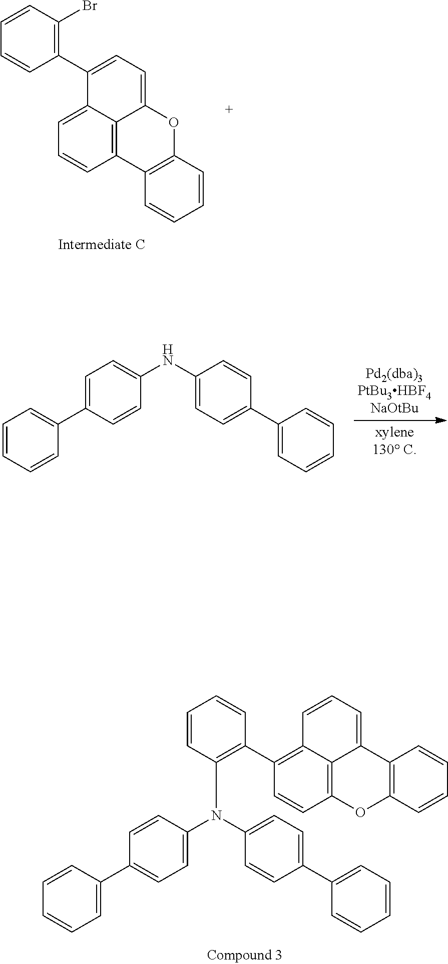 [SMILES: Brc1ccccc1-c1ccc2c3c(cccc13)-c1ccccc1O2 ; c1ccc(-c2ccc(Nc3ccc(-c4ccccc4)cc3)cc2)cc1 ; c1ccc(-c2ccc(N(c3ccc(-c4ccccc4)cc3)c3ccccc3-c3ccc4c5c(cccc35)-c3ccccc3O4)cc2)cc1]

Compound 3 was obtained in the same manner as in Synthesis Example 1 except that intermediate C was used instead of intermediate A used in Synthesis Example 1.

As a result of mass spectrum analysis, the obtained compound was compound 3, in which m/e=614 with respect to the molecular weight of 613.76.

### Synthesis Example 4: Synthesis of Compound 4

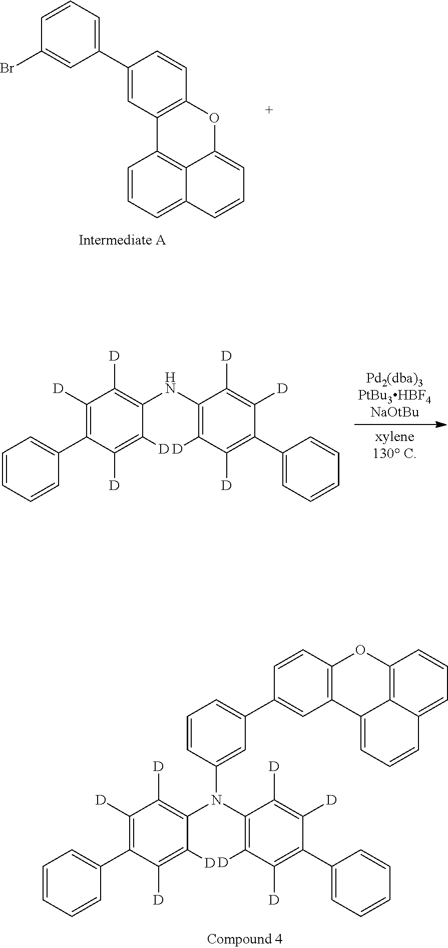 [SMILES: Brc1cccc(-c2ccc3c(c2)-c2cccc4cccc(c24)O3)c1 ; [2H]c1c([2H])c(-c2ccccc2)c([2H])c([2H])c1Nc1c([2H])c([2H])c(-c2ccccc2)c([2H])c1[2H] ; [2H]c1c([2H])c(N(c2cccc(-c3ccc4c(c3)-c3cccc5cccc(c35)O4)c2)c2c([2H])c([2H])c(-c3ccccc3)c([2H])c2[2H])c([2H])c([2H])c1-c1ccccc1]

Compound 4 was obtained in the same manner as in Synthesis Example 1 except that N-([1,1′-biphenyl]-4-yl-2,3,5,6-d4)-[1,1′-biphenyl-2,3,5,6-d4]-4-amine was used instead of N-[1,1′-biphenyl]-4-yl-[1,1′-biphenyl]-4-amine used in Synthesis Example 1.

As a result of mass spectrum analysis, the obtained compound was compound 4, in which m/e=622 with respect to the molecular weight of 621.81.

### Synthesis Example 5: Synthesis of Compound 5

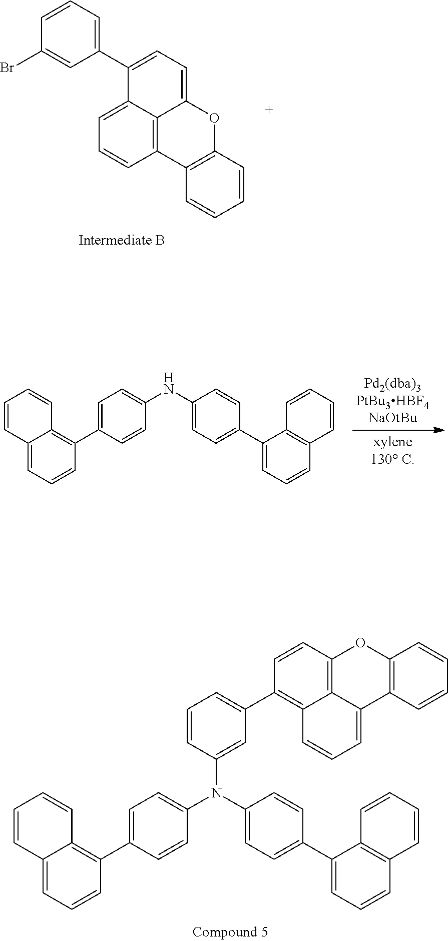 [SMILES: Brc1cccc(-c2ccc3c4c(cccc24)-c2ccccc2O3)c1 ; c1ccc2c(-c3ccc(Nc4ccc(-c5cccc6ccccc56)cc4)cc3)cccc2c1 ; c1cc(-c2ccc3c4c(cccc24)-c2ccccc2O3)cc(N(c2ccc(-c3cccc4ccccc34)cc2)c2ccc(-c3cccc4ccccc34)cc2)c1]

Compound 5 was obtained in the same manner as in Synthesis Example 2 except that 4-(1-naphthalenyl)-N-[4-(1-naphthalenyl)phenyl]benzeneamine was used instead of N-[1,1′-biphenyl]-4-yl-[1,1′-biphenyl]-4-amine used in Synthesis Example 2.

As a result of mass spectrum analysis, the obtained compound was compound 5, in which m/e=714 with respect to the molecular weight of 713.88.

### Synthesis Example 6: Synthesis of Compound 6

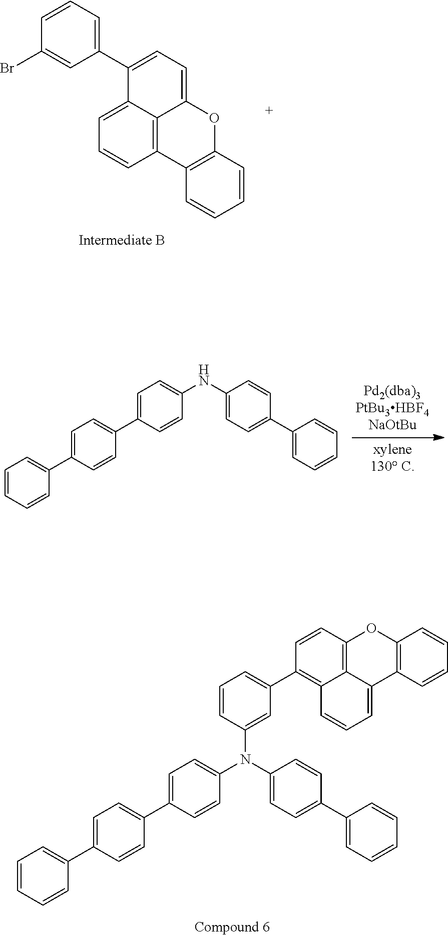 [SMILES: Brc1cccc(-c2ccc3c4c(cccc24)-c2ccccc2O3)c1 ; c1ccc(-c2ccc(Nc3ccc(-c4ccc(-c5ccccc5)cc4)cc3)cc2)cc1 ; c1ccc(-c2ccc(-c3ccc(N(c4ccc(-c5ccccc5)cc4)c4cccc(-c5ccc6c7c(cccc57)-c5ccccc5O6)c4)cc3)cc2)cc1]

Compound 6 was obtained in the same manner as in Synthesis Example 2 except that N-[1,1′-biphenyl]-4-yl-[1,1′:4′,1″-terphenyl]-4-amine was used instead of N-[1,1′-biphenyl]-4-yl-[1,1′-biphenyl]-4-amine used in Synthesis Example 2.

As a result of mass spectrum analysis, the obtained compound was compound 6, in which m/e=670 with respect to the molecular weight of 689.86.

### REFERENCE SIGNS LIST

1, 11, 12: Organic EL device
2: Substrate
3: Anode
4: Cathode
5: Light emitting layer
6: Hole transporting band (hole transporting layer)
6a: Hole injecting layer
6b: First hole transporting layer
6c: Second hole transporting layer
6d: Third hole transporting layer
7: Electron transporting band (electron transporting layer)
7a: First electron transporting layer
7b: Second electron transporting layer
10, 20, 30: Light emitting unit

1. A compound represented by formula (1) below: 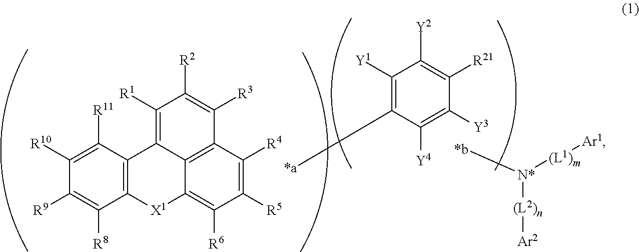
wherein X¹ is an oxygen atom or a sulfur atom;
one selected from R¹ to R⁶ and R⁸ to R¹¹ is a single bond bonded to *a;
R¹ to R⁶ and R⁸ to R¹¹ that are not the single bond are each independently a hydrogen atom, an unsubstituted alkyl group having 1 to 30 carbon atoms, or an unsubstituted aryl group having 6 to 12 ring carbon atoms;
each combination of adjacent moieties of R¹ to R⁶ and R⁸ to R¹¹ that are not the single bond forms no ring, with the adjacent moieties not bonded to each other;
R²¹ is a hydrogen atom;
one selected from Y¹ to Y⁴ is a single bond bonded to *b;
Y¹ to Y⁴ that are not the single bond are hydrogen atoms;
N* is a central nitrogen atom;
m is 0 or 1;
n is 0 or 1;
when m is 0, Ar¹ is directly bonded to the central nitrogen atom *;
when n is 0, Ar² is directly bonded to the central nitrogen atom *;
L¹ and L² are each independently a substituted or unsubstituted phenylene group, a substituted or unsubstituted naphthylene group, or a substituted or unsubstituted biphenylene group;
a substituent of the phenylene group, the naphthylene group, and the biphenylene group which L¹ and L² may take is selected from an unsubstituted alkyl group having 1 to 6 carbon atoms and an aryl group having 6 to 12 ring carbon atoms, and substituents are not bonded to each other and form no ring; and
Ar¹ and Ar² are each independently represented by any of formulae (1-a) to (1-d) below; 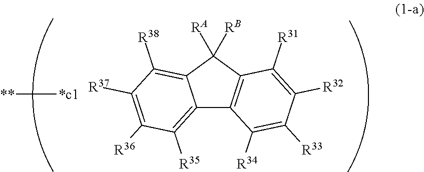
in formula (1-a),
** represents a bonding site to one or both of L¹ and L²;
one selected from R³¹ to R³⁸, R^{A}, and R^{B} is a single bond bonded to *c1, or one selected from R^{A} and R^{B} is a divalent group bonded to *c1;
R³¹ to R³⁸ that are not the single bond are each independently a hydrogen atom, an unsubstituted alkyl group having 1 to 30 carbon atoms, or an unsubstituted aryl group having 6 to 12 ring carbon atoms;
one combination of adjacent moieties among R³¹ to R³⁸ that are not the single bond forms an unsubstituted benzene ring, with the adjacent moieties bonded to each other, or each combination of adjacent moieties of R³¹ to R³⁸ forms no ring, with the adjacent moieties not bonded to each other;
R^{A} and R^{B} that are not the single bond and are not the group bonded to *c1 are a substituted or unsubstituted alkyl group having 1 to 30 carbon atoms, a substituted or unsubstituted aryl group having 6 to 30 ring carbon atoms, or a substituted or unsubstituted heteroaryl group having 5 to 30 ring atoms, and at least one of R^{A} and R^{B} is a substituted or unsubstituted aryl group having 6 to 30 ring carbon atoms;
the divalent group bonded to *c1 represented by R^{A} and R^{B} is a substituted or unsubstituted alkylene group having 1 to 30 carbon atoms, a substituted or unsubstituted arylene group having 6 to 30 ring carbon atoms, or a substituted or unsubstituted heteroarylene group having 5 to 30 ring atoms; and
R^{A} and R^{B} that are not the single bond and are not the group bonded to *c1 are bonded to each other to form a ring, or are not bonded to each other and form no ring; 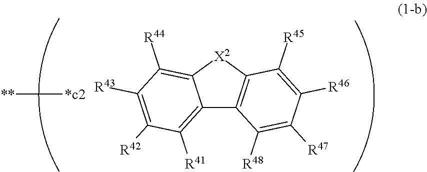
in formula (1-b),
** represents a bonding site to one or both of L¹ and L²;
X² represents an oxygen atom or a sulfur atom;
one selected from R⁴¹ to R⁴⁸ is a single bond bonded to *c2;
R⁴¹ to R⁴⁸ that are not the single bond are each independently a hydrogen atom, an unsubstituted alkyl group having 1 to 30 carbon atoms, or an unsubstituted aryl group having 6 to 12 ring carbon atoms; and
one combination of adjacent moieties among R⁴¹ to R⁴⁸ that are not the single bond forms an unsubstituted benzene ring, with the adjacent moieties bonded to each other, or each combination of adjacent moieties among R⁴¹ to R⁴⁸ forms no ring, with the adjacent moieties not bonded to each other; 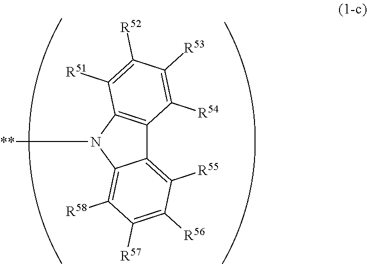
In formula (1-c),
** represents a bonding site to one or both of L¹ and L².
R⁵¹ to R⁵⁸ are each independently a hydrogen atom, an unsubstituted alkyl group having 1 to 30 carbon atoms, or an unsubstituted aryl group having 6 to 12 ring carbon atoms.
One combination of adjacent moieties among R⁵¹ to R⁵⁸ that are not the single bond forms an unsubstituted benzene ring, with the adjacent moieties bonded to each other, or each combination of adjacent moieties among R⁵¹ to R⁵⁸ forms no ring, with the adjacent moieties not bonded to each other.
**—Z (1-d)
in formula (1-d),
** represents a bonding site to one or both of L¹ and L²;
Z is a hydrogen atom or a substituted or unsubstituted aryl group consisting only of a 6-membered ring and having 6 to 30 ring carbon atoms;
substituents on the aryl group represented by Z are bonded to each other to form a monocyclic ring, are bonded to each other to form a condensed ring, or are not bonded to each other and form no ring; and
the condensed ring is selected from a naphthalene ring, an anthracene ring, a phenanthrene ring, and a phenalene ring.

2. The compound according to claim 1, represented by formula (1A) below: 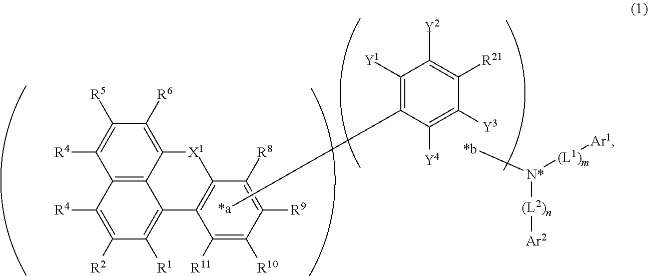
wherein X¹, R¹ to R⁶, R⁸ to R¹¹, R²¹, Y¹ to Y⁴, N*, L¹, L², Ar¹, Ar², *a, *b, m, and n are as defined for the formula (1), provided that one selected from R⁸ to R¹¹ is a single bond bonded to *a.

3. The compound according to claim 1, represented by formula (1B) below: 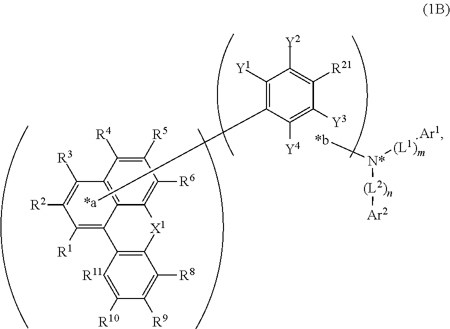
wherein X¹, R¹ to R⁶, R⁸ to R¹¹, R²¹, Y¹ to Y⁴, N*, L¹, L², Ar¹, Ar², *a, *b, m, and n are as defined for the formula (1), provided that one selected from R¹ to R⁶ is a single bond bonded to *a.

4. The compound according to claim 1, represented by formula (1C) below: 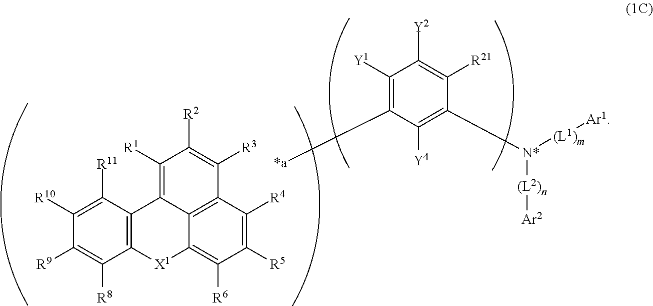
wherein X¹, R¹ to R⁶, R⁸ to R¹¹, R²¹, Y¹, Y², Y⁴, N*, L¹, L², Ar¹, Ar², *a, m, and n are as defined for the formula (1).

5. The compound according to claim 1, represented by formula (1D) below: 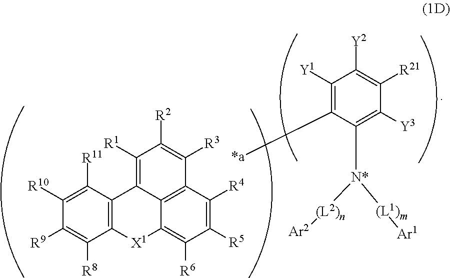
wherein X¹, R¹ to R⁶, R⁸ to R¹¹, R²¹, Y¹ to Y³, N*, L¹, L², Ar¹, Ar², *a, m, and n are as defined for the formula (1).

6. (canceled)

7. The compound according to claim 1, represented by formula (1F) below: 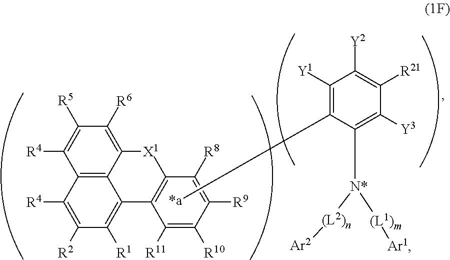
wherein X¹, R¹ to R⁶, R⁸ to R¹¹, R²¹, Y¹ to Y³, N*, L¹, L², Ar¹, Ar², *a, m, and n are as defined for the formula (1).

8. (canceled)

9. The compound according to claim 1, represented by formula (1H) below: 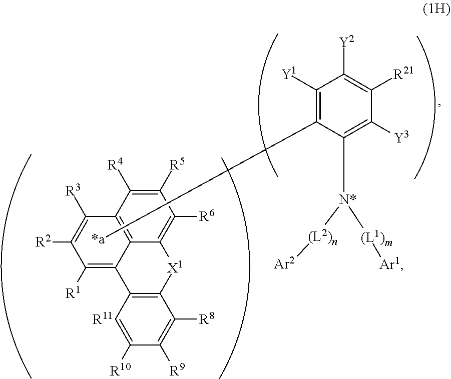
wherein X¹, R¹ to R⁶, R⁸ to R¹¹, R²¹, Y¹ to Y³, N*, L¹, L², Ar¹, Ar², *a, m, and n are as defined for the formula (1), provided that one selected from R¹ to R⁶ is a single bond bonded to *a.

10. The compound according to claim 1, represented by formula (1J1) below: 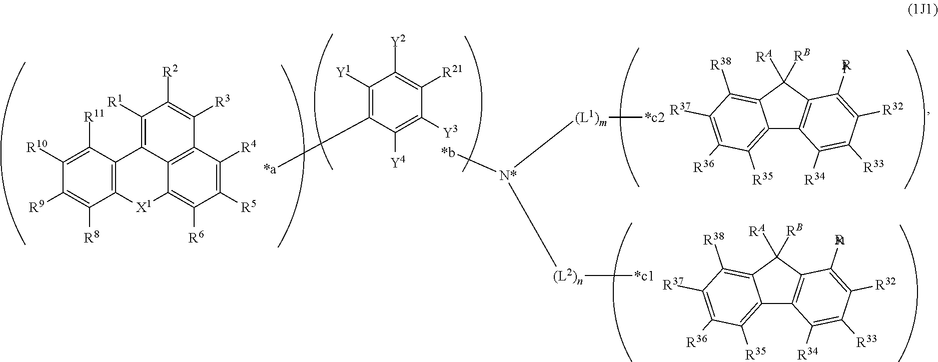
wherein X¹, R¹ to R⁶, R⁸ to R¹¹, R²¹, Y¹ to Y⁴, N*, L¹, L², R³¹ to R³⁸, R^{A}, R^{B}, *c1, m, and n are as defined for the formula (1), provided that R³¹ to R³⁸, R^{A}, R^{B}, and *c1 corresponding to Ar¹ and R³¹ to R³⁸, R^{A}, R^{B}, and *c1 corresponding to Ar² are the same as or different from each other.

11-19. (canceled)

20. The compound according to claim 1, wherein Ar¹ and Ar² are each independently represented by any one of the formula (1-a), (1-b), or (1-c), or formulae (1-d1) to (1-d4) below, 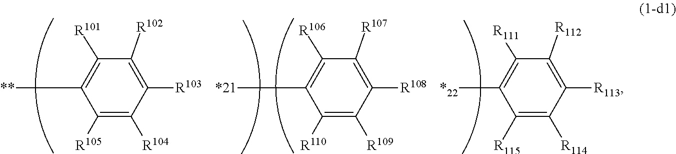
in formula (1-d1),
** represents a bonding site to one or both of L¹ and L²;
R¹⁰¹ to R¹⁰⁵, R¹⁰⁶ to R¹¹⁰, and R¹¹¹ to R¹¹⁵ are each independently a hydrogen atom, an unsubstituted alkyl group having 1 to 6 carbon atoms, or an unsubstituted aryl group having 6 to 12 ring carbon atoms, provided that one selected from R¹⁰¹ to R¹⁰⁵ is a single bond bonded to *21, and one selected from R¹⁰⁶ to R¹¹⁰ is a single bond bonded to *22;
each combination of adjacent moieties among R¹⁰¹ to R¹⁰⁵ that are not the single bond forms no ring, with the adjacent moieties not bonded to each other;
each combination of adjacent moieties among R¹⁰⁶ to R¹¹⁰ that are not the single bond forms no ring, with the adjacent moieties not bonded to each other;
p is 0 or 1;
q is 0 or 1;
when p is 0, and q is 1, *21 is a bonding site to one or both of L¹ and L²; and
when p is 1, and q is 0, one selected from R¹⁰¹ to R¹⁰⁵ is a single bond bonded to *22; 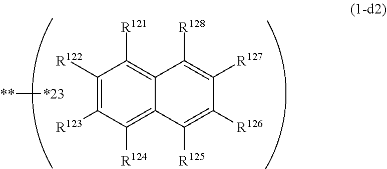
in formula (1-d2),
** represents a bonding site to one or both of L¹ and L²; and
R¹²¹ to R¹²⁸ are each independently a hydrogen atom, a substituted or unsubstituted alkyl group having 1 to 6 carbon atoms, or a substituted or unsubstituted aryl group having 6 to 12 ring carbon atoms, provided that one selected from R¹²¹ to R¹²⁸ is a single bond bonded to *23, and each combination of adjacent moieties among R¹²¹ to R¹²⁸ that are not the single bond forms no ring, with the adjacent moieties not bonded to each other; 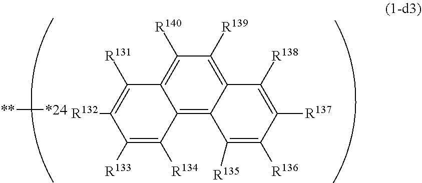
in formula (1-d3),
** represents a bonding site to one or both of L¹ and L²; and
R¹³¹ to R¹⁴⁰ are each independently a hydrogen atom, a substituted or unsubstituted alkyl group having 1 to 6 carbon atoms, or a substituted or unsubstituted aryl group having 6 to 12 ring carbon atoms, provided that one selected from R¹³¹ to R¹⁴⁰ is a single bond bonded to *24, and each combination of adjacent moieties among R¹³¹ to R¹⁴⁰ that are not the single bond forms no ring, with the adjacent moieties not bonded to each other; 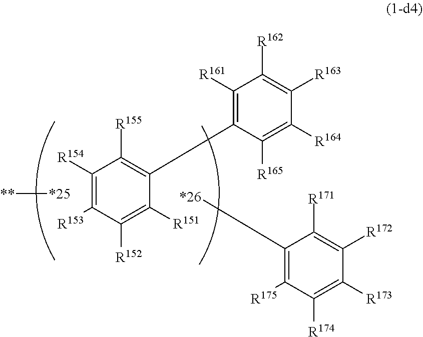
in formula (1-d4),
** represents a bonding site to one or both of L¹ and L²;
R¹⁵¹ to R¹⁵⁵ are each independently a hydrogen atom, an unsubstituted alkyl group having 1 to 6 carbon atoms, or an unsubstituted phenyl group, provided that one selected from R¹⁵¹ to R¹⁵⁵ is a single bond bonded to *25, and another one selected from R¹⁵¹ to R¹⁵⁵ is a single bond bonded to *26;
each combination of adjacent moieties among R¹⁵¹ to R¹⁵⁵ that are neither the single bond bonded to *25 nor the single bond bonded to *26 forms no ring, with the adjacent moieties not bonded to each other;
R¹⁶¹ to R¹⁶⁵ and R¹⁷¹ to R¹⁷⁵ are each independently a hydrogen atom or an unsubstituted alkyl group having 1 to 6 carbon atoms, provided that at least one combination of adjacent two moieties among R¹⁶¹ to R¹⁶⁵ forms one or more unsubstituted benzene rings, with the adjacent moieties bonded to each other, or each combination of adjacent moieties among R¹⁶¹ to R¹⁶⁵ forms no ring, with the adjacent moieties not bonded to each other. At least one combination of adjacent two moieties among R¹⁷¹ to R¹⁷⁵ forms one or more unsubstituted benzene rings, with the adjacent moieties bonded to each other, or each combination of adjacent moieties among R¹⁷¹ to R¹⁷⁵ forms no ring, with the adjacent moieties not bonded to each other.

21. The compound according to claim 20, wherein at least one of Ar¹ or Ar² in the formula (1) is represented by the formula (1-d1).

22. The compound according to claim 20, wherein Ar¹ and Ar² in the formula (1) are represented by the formula (1-d1).

23-26. (canceled)

27. The compound according to claim 1, wherein m and n are 0 in the formula (1).

28. The compound according to claim 1, wherein the compound represented by the formula (1) includes at least one deuterium atom.

29. (canceled)

30. A material for an organic electroluminescent device, wherein the material is a hole transporting layer material comprising the compound according to claim 1.

31. An organic electroluminescent device comprising:
a cathode;
an anode; and
an organic layer between the cathode and the anode, wherein
the organic layer includes a light emitting layer, and
at least one layer of the organic layer includes the compound according to claim 1.

32. The organic electroluminescent device according to claim 31, wherein
the organic layer includes a hole transporting band between the anode and the light emitting layer, and
the hole transporting band includes the compound.

33. The organic electroluminescent device according to claim 32, wherein
the hole transporting band includes a first hole transporting layer on the anode side and a second hole transporting layer on the cathode side, and
at least one of the first hole transporting layer or the second hole transporting layer includes the compound.

34. (canceled)

35. The organic electroluminescent device according to claim 33, wherein the light emitting layer and the second hole transporting layer directly contact with each other.

36. (canceled)

37. The organic electroluminescent device according to claim 31, wherein the light emitting layer includes a layer containing a light emitting compound emitting fluorescent light having a main peak wavelength of 500 nm or less.

38. The organic electroluminescent device according to claim 31, wherein the light emitting layer is a single layer.

39. (canceled)
